(19) United States
(12) Patent Application Publication
(10) Pub. No.: US 2023/0363266 A1
(43) Pub. Date: Nov. 9, 2023

(54) NOVEL COMPOUND AND ORGANIC LIGHT EMITTING DEVICE COMPRISING THE SAME

(71) Applicant: LG CHEM, LTD., Seoul (KR)

(72) Inventors: Minjun KIM, Daejeon (KR); Dong Hoon LEE, Daejeon (KR); Sang Duk SUH, Daejeon (KR); Young Seok KIM, Daejeon (KR); Da Jung LEE, Daejeon (KR)

(21) Appl. No.: 18/026,175

(22) PCT Filed: Dec. 14, 2021
(86) PCT No.: PCT/KR2021/018987

§ 371 Date: Mar. 14, 2023

(30) Foreign Application Priority Data
Dec. 14, 2020 (KR) 10-2020-0174596
Dec. 14, 2021 (KR) 10-2021-0178415

(51) Int. Cl. H10K 85/60; C07D 498/04; C07D 513/04; C09K 11/06; H10K 101/00; H10K 50/18; H10K 50/11

(52) U.S. Cl. CPC H10K 85/657; H10K 85/636; C07D 498/04; H10K 85/6576; H10K 85/615; C07D 513/04; C09K 11/06; H10K 85/6574; H10K 85/633; H10K 85/654; H10K 85/626; H10K 2101/90; C09K 2211/1018; H10K 50/181; H10K 50/11

(57) ABSTRACT

Provided is a novel compound of Chemical Formula 1:

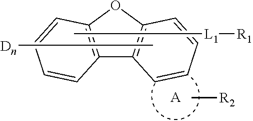

wherein: A is a thiazole ring or an oxazole ring fused with an adjacent ring; L₁ is a single bond, substituted or unsubstituted C₆₋₆₀ arylene, or substituted or unsubstituted C₂₋₆₀ heteroarylene containing at least of N, O and S; R₁ is

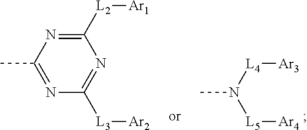

Ar₁ to Ar₄ are each independently substituted or unsubstituted C₆₋₆₀ aryl or substituted or unsubstituted C₂₋₆₀ heteroaryl containing at least one of N, O and S; L₂ to L₅ are each independently a single bond, substituted or unsubstituted C₆₋₆₀ arylene, or substituted or unsubstituted C₂₋₆₀ heteroarylene containing at least one of N, O and S; R₂ is a substituted or unsubstituted C₆₋₆₀ aryl or substituted or unsubstituted C₂₋₆₀ heteroaryl containing at least of N, O and S; D is deuterium; and n is an integer of 0 to 5, and an organic light emitting device comprising the same.

### CROSS-REFERENCE TO RELATED APPLICATION(S)

This application is a National Stage Application of International Application No. PCT/KR2021/018987 filed on Dec. 14, 2021, which claims the benefit of Korean Patent Application No. 10-2020-0174596 filed on Dec. 14, 2020, and Korean Patent Application No. 10-2021-0178415 filed on Dec. 14, 2021 in the Korean Intellectual Property Office, the disclosures of which are incorporated herein by reference in their entirety.

### TECHNICAL FIELD

The present disclosure relates to a novel compound and to an organic light emitting device comprising the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer can be formed of a hole injection layer, a hole transport layer, a light emitting layer, an electron transport layer, an electron injection layer and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuing need for the development of new materials for the organic materials used in the organic light emitting devices as described above.

### PRIOR ART LITERATURE

### Patent Literature

(Patent Literature 1) Korean Unexamined Patent Publication No. 10-2000-0051826

### BRIEF DESCRIPTION

### Technical Problem

The present disclosure relates to a novel organic light emitting material and an organic light emitting device comprising the same.

### Technical Solution

In the present disclosure, provided is a compound of the following Chemical Formula 1:

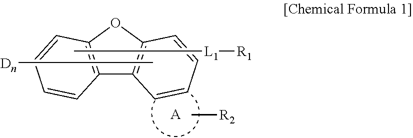

wherein in Chemical Formula 1:
A is a thiazole ring or an oxazole ring fused with an adjacent ring;
L₁ is a single bond, substituted or unsubstituted C₆₋₆₀ arylene, or substituted or unsubstituted C₂₋₆₀ heteroarylene containing at least one heteroatom selected from the group consisting of N, O and S;
R₁ is

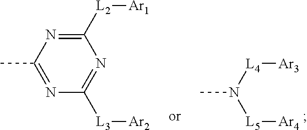

Ar₁ to Ar₄ are each independently substituted or unsubstituted C₆₋₆₀ aryl or substituted or unsubstituted C₂₋₆₀ heteroaryl containing at least one heteroatom selected from the group consisting of N, O and S;
L₂ to L₅ are each independently a single bond, substituted or unsubstituted C₆₋₆₀ arylene, or substituted or unsubstituted C₂₋₆₀ heteroarylene containing at least one heteroatom selected from the group consisting of N, O and S;
R₂ is a substituted or unsubstituted C₆₋₆₀ aryl or substituted or unsubstituted C₂₋₆₀ heteroaryl containing at least one heteroatom selected from the group consisting of N, O and S;
D is deuterium; and
n is an integer of 0 to 5.

In addition, also provided is an organic light emitting device including: a first electrode; a second electrode that is provided opposite to the first electrode; and one or more organic material layers that are provided between the first electrode and the second electrode, wherein at least one layer of the organic material layers includes at least one compound of Chemical Formula 1.

### Advantageous Effects

The compound of Chemical Formula 1 can be used as a material of an organic material layer of an organic light emitting device, and can improve the efficiency, achieve low driving voltage and/or improve lifetime characteristics in the organic light emitting device. In particular, the compound of Chemical Formula 1 can be used as a material for hole injection, hole transport, light emission, electron transport, and/or electron injection.

### DESCRIPTION OF THE DRAWINGS

FIG. 1 shows an example of an organic light emitting device including a substrate 1, an anode 2, a light emitting layer 3, and a cathode 4.

FIG. 2 shows an example of an organic light emitting device including a substrate 1, an anode 2, a hole injection layer 5, a hole transport layer 6, an electron blocking layer 7, a light emitting layer 3, a hole blocking layer 8, an electron injection and transport layer 9, and a cathode 4.

### DETAILED DESCRIPTION

Hereinafter, embodiments of the present disclosure will be described in more detail to facilitate understanding of the invention.

In the present disclosure, provided is a compound of Chemical Formula 1.

As used herein, the notation [symbol] or [symbol] means a bond linked to another substituent group.

As used herein, the term “substituted or unsubstituted” means being unsubstituted or substituted with one or more substituents selected from the group consisting of deuterium, a halogen group; a nitrile group, a nitro group, a hydroxy group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, and a heteroaryl group containing at least one of N, O and S atoms, or being unsubstituted or substituted with a substituent to which two or more substituents of the above-exemplified substituents are connected. For example, “a substituent in which two or more substituents are connected” can be a biphenyl group. Namely, a biphenyl group can be an aryl group, or it can also be interpreted as a substituent in which two phenyl groups are connected.

In the present disclosure, the carbon number of a carbonyl group is not particularly limited, but is preferably 1 to 40. Specifically, the carbonyl group can be a substituent having the following structural formulas, but is not limited thereto:

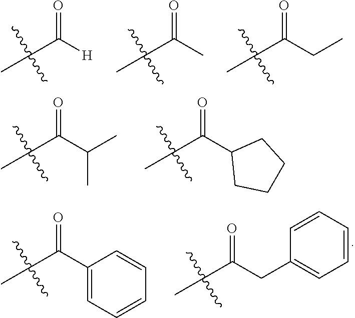

In the present disclosure, an ester group can have a structure in which oxygen of the ester group can be substituted by a straight-chain, branched-chain, or cyclic alkyl group having 1 to 25 carbon atoms, or an aryl group having 6 to 25 carbon atoms. Specifically, the ester group can be a substituent having the following structural formulas, but is not limited thereto:

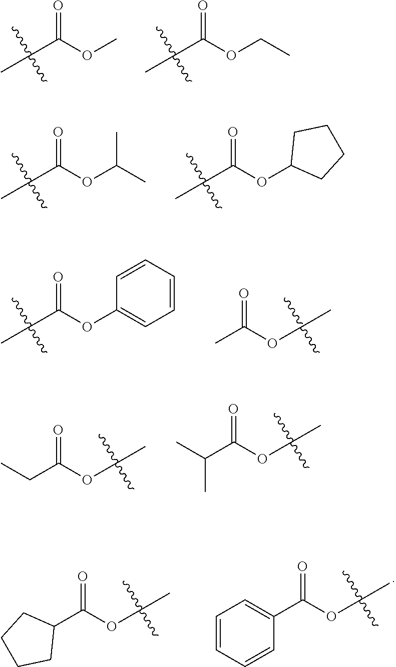

In the present disclosure, the carbon number of an imide group is not particularly limited, but is preferably 1 to 25. Specifically, the imide group can be a substituent having the following structural formulas, but is not limited thereto:

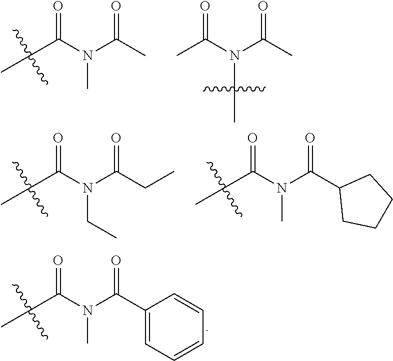

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but is not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluorine, chlorine, bromine, or iodine.

In the present disclosure, the alkyl group can be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present disclosure, the alkenyl group can be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, a cycloalkyl group is not particularly limited, but the carbon number thereof is preferably 3 to 60. According to one embodiment, the carbon number of the cycloalkyl group is 3 to 30. According to another embodiment, the carbon number of the cycloalkyl group is 3 to 20. According to still another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it can be a monocyclic aryl group or a polycyclic aryl group. According to one embodiment, the carbon number of the aryl group is 6 to 30. According to one embodiment, the carbon number of the aryl group is 6 to 20. The aryl group can be a phenyl group, a biphenyl group, a terphenyl group or the like as the monocyclic aryl group, but is not limited thereto. The polycyclic aryl group includes a naphthyl group, an anthracenyl group, a phenanthryl group, a pyrenyl group, a perylenyl group, a chrysenyl group, fluorenyl group or the like, but is not limited thereto.

In the present disclosure, the fluorenyl group can be substituted, and two substituents can be linked with each other to form a spiro structure. In the case where the fluorenyl group is substituted,

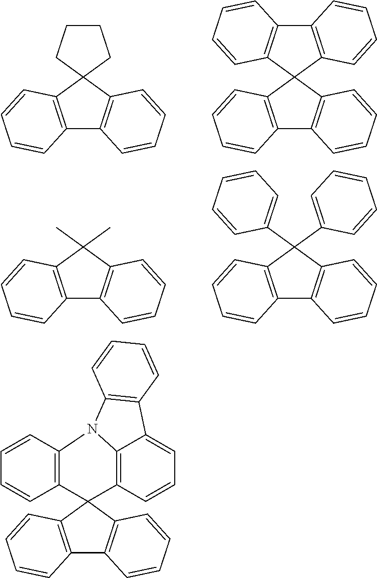

and the like can be formed. However, the structure is not limited thereto.

In the present disclosure, a heteroaryl group is a heteroaryl group containing one or more of O, N, Si and S as a heteroatom, and the carbon number thereof is not particularly limited, but is preferably 2 to 60. According to an exemplary embodiment, the heteroaryl group has 6 to 30 carbon atoms. According to an exemplary embodiment, the heteroaryl group has 6 to 20 carbon atoms. Examples of the heteroaryl group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the aforementioned examples of the aryl group. In the present disclosure, the alkyl group in the aralkyl group, the alkylaryl group and the alkylamine group is the same as the aforementioned examples of the alkyl group. In the present disclosure, the heteroaryl in the heteroarylamine can be applied to the aforementioned description of the heteroaryl group. In the present disclosure, the alkenyl group in the aralkenyl group is the same as the aforementioned examples of the alkenyl group. In the present disclosure, the aforementioned description of the aryl group can be applied except that the arylene is a divalent group. In the present disclosure, the aforementioned description of the heteroaryl group can be applied except that the heteroarylene is a divalent group. In the present disclosure, the aforementioned description of the aryl group or cycloalkyl group can be applied except that the hydrocarbon ring is not a monovalent group but formed by combining two substituent groups. In the present disclosure, the aforementioned description of the heteroaryl group can be applied, except that the heteroaryl is not a monovalent group but formed by combining two substituent groups.

Preferably, the Chemical Formula 1 can be any one of the following Chemical Formulae 1-1 to 1-4:

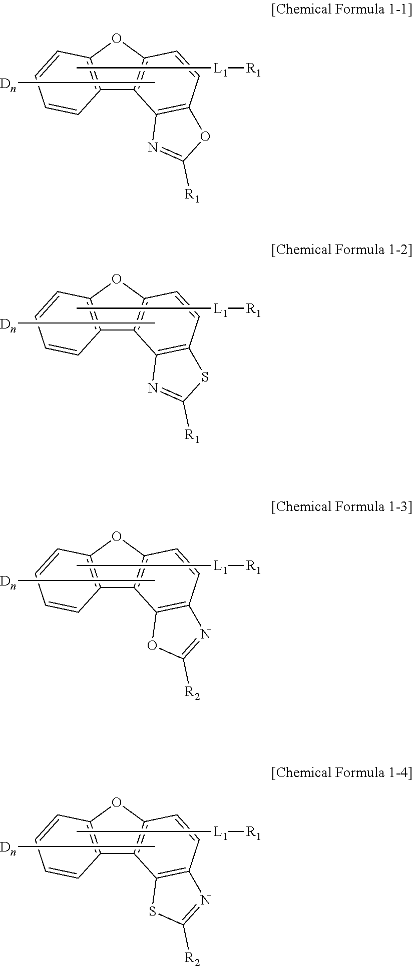

wherein in the Chemical Formulae 1-1 to 1-4,
R₁, R₂, L₁, D and n are as defined in the Chemical Formula 1.

Preferably, L₁ can be a single bond, a substituted or unsubstituted C₆₋₂₀ arylene; or a substituted or unsubstituted C₂-20 heteroarylene containing at least one heteroatom selected from the group consisting of N, O and S.

More preferably, L₁ can be a single bond, phenylene, biphenyldiyl, or naphthalenediyl.

Most preferably, L₁ can be a single bond or any one selected from the group consisting of:

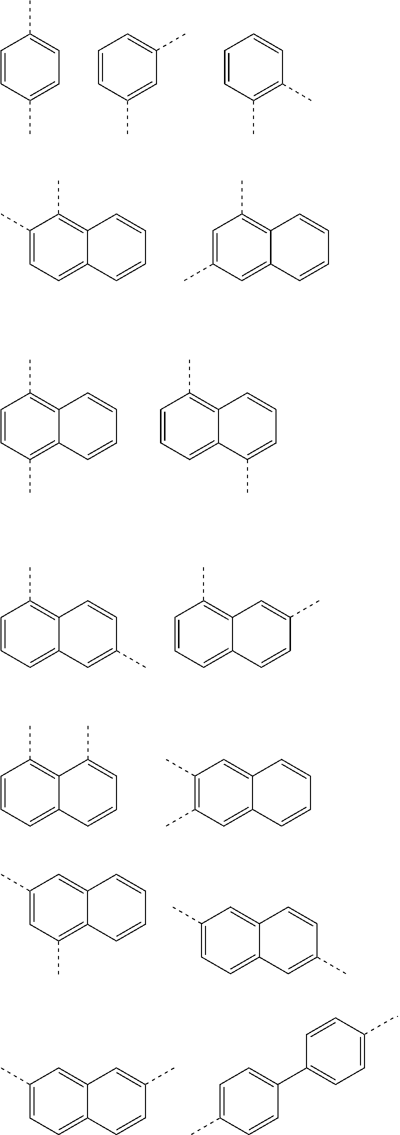

Preferably, Ar₁ and Ar₂ can each independently be a substituted or unsubstituted C₆₋₂₀ aryl or substituted or unsubstituted C₂₋₂₀ heteroaryl containing at least one heteroatom selected from the group consisting of N, O and S.

More preferably, Ar₁ and Ar₂ can each independently be phenyl, biphenylyl, naphthyl, phenanthrenyl, dibenzofuranyl, or dibenzothiophenyl.

Most preferably, Ar₁ and Ar₂ can each independently be any one selected from the group consisting of:

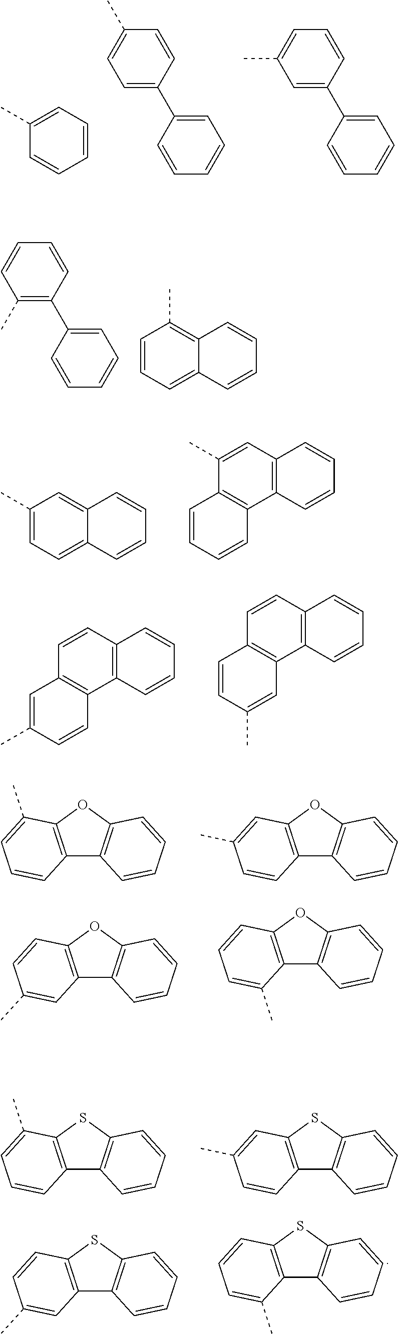

Preferably, Ar₃ and Ar₄ can each independently be a substituted or unsubstituted C₆₋₂₀ aryl or substituted or unsubstituted C₂₋₂₀ heteroaryl containing at least one heteroatom selected from the group consisting of N, O and S.

More preferably, Ar₃ and Ar₄ can each independently be phenyl, biphenylyl, terphenylyl, naphthyl, phenanthrenyl, dibenzofuranyl, dibenzothiophenyl, phenyl carbazolyl, or phenyl naphthyl.

Most preferably, Ar₃ and Ar₄ can each independently be any one selected from the group consisting of:

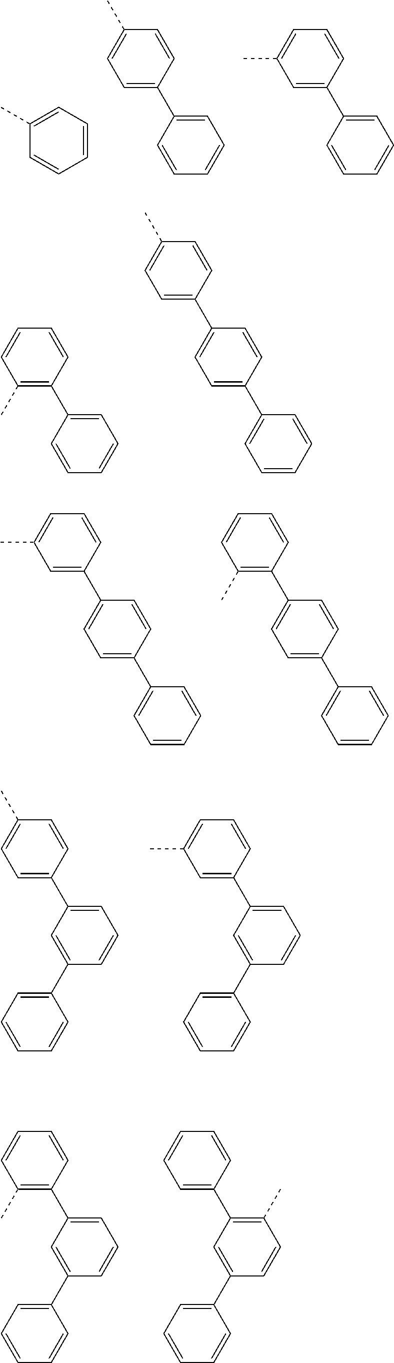

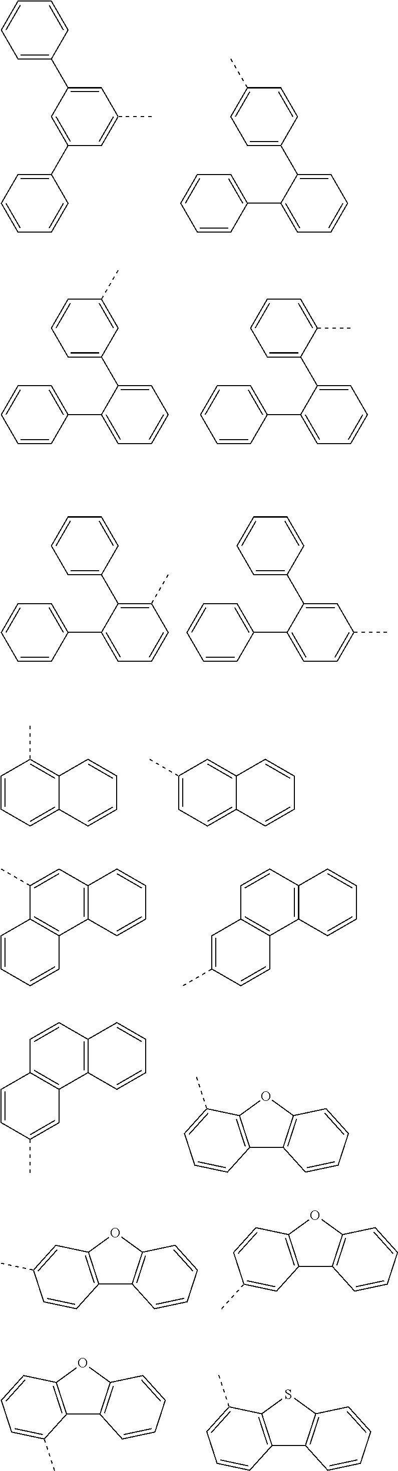

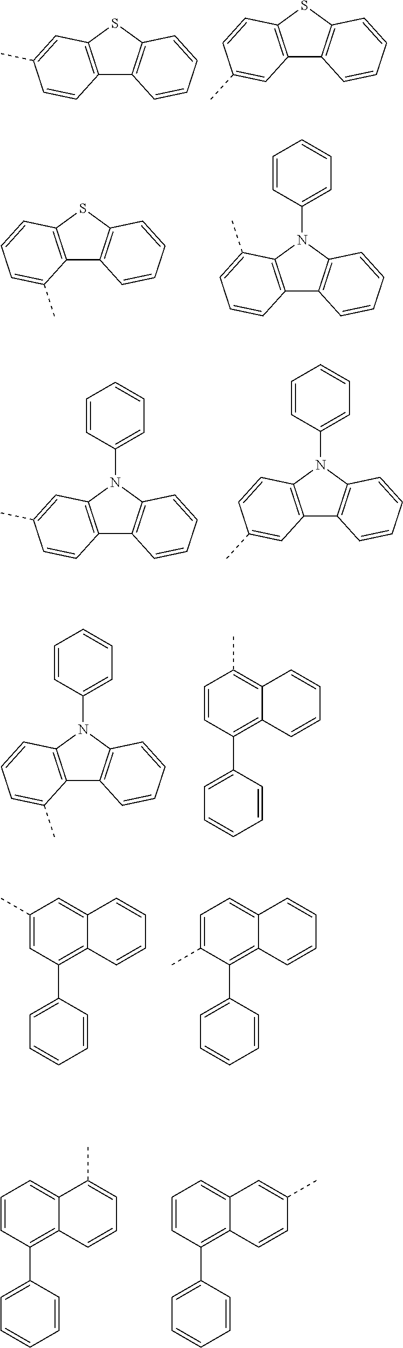

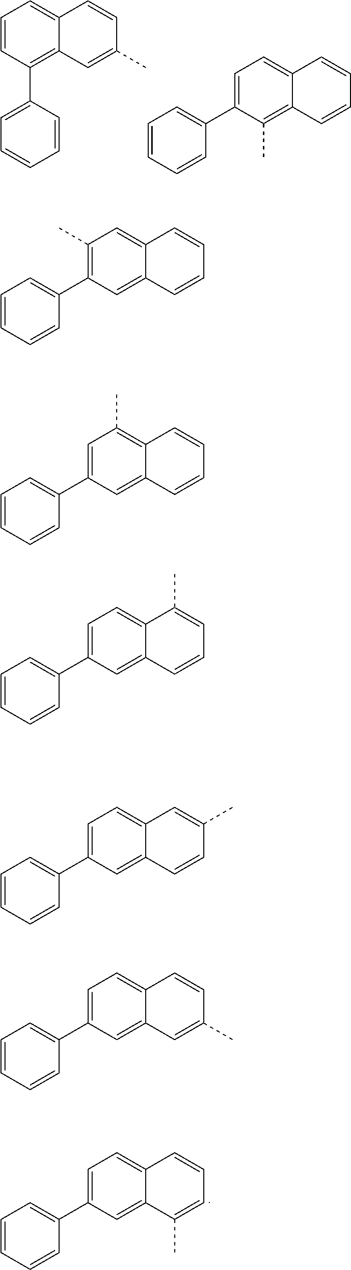

Preferably, L₂ and L₃ can each independently be a single bond or substituted or unsubstituted C₆₋₂₀ arylene.

More preferably, L₂ and L₃ can each independently be a single bond, phenylene, or naphthalenediyl.

Most preferably, L₂ and L₃ can each independently be a single bond or any one selected from the group consisting of:

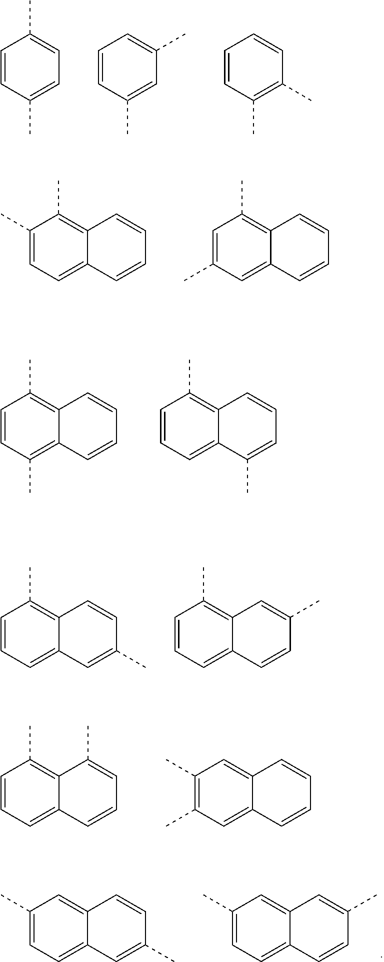

Preferably, L₄ and L₅ can each independently be a single bond, a substituted or unsubstituted C₆₋₂₀ arylene; or a substituted or unsubstituted C₂₋₂₀ heteroarylene containing at least one heteroatom selected from the group consisting of N, O and S.

More preferably, L₄ and L₅ can each independently be a single bond, phenylene, biphenyldiyl, naphthalenediyl, or carbazolediyl.

Most preferably, L₄ and L₅ can each independently be a single bond or any one selected from the group consisting of:

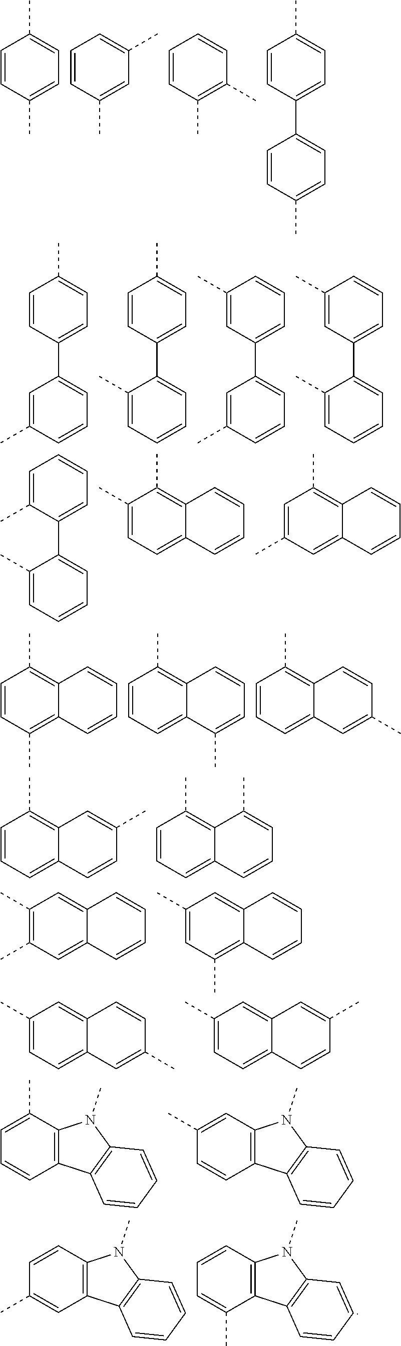

Preferably, at least one of Ar₁ and Ar₂ can be a substituted or unsubstituted C₆₋₆₀ aryl, more preferably, at least one of Ar₁ and Ar₂ can be a substituted or unsubstituted C₆₋₂₀ aryl, more preferably, at least one of Ar₁ and Ar₂ can be an unsubstituted C₆₋₂₀ aryl, and most preferably, at least one of Ar₁ and Ar₂ can be phenyl or naphthyl.

Preferably, at least one of Ar₃ and Ar₄ can be a substituted or unsubstituted C₆₋₆₀ aryl, more preferably, at least one of Ar₃ and Ar₄ can be a substituted or unsubstituted C₆₋₂₀ aryl, more preferably, at least one of Ar₃ and Ar₄ can be an unsubstituted C₆₋₂₀ aryl, and most preferably, at least one of Ar₃ and Ar₄ can be phenyl, biphenylyl, or naphthyl.

Meanwhile, R₂ is a substituent of ring A.

Preferably, R₂ can be a substituted or unsubstituted C₆₋₂₀ aryl or a substituted or unsubstituted C₂₋₂₀ heteroaryl containing at least one heteroatom selected from the group consisting of N, O and S.

More preferably, R₂ can be phenyl, biphenylyl, naphthyl, dibenzofuranyl, or dibenzothiophenyl.

Most preferably, R₂ can be any one selected from the group consisting of:

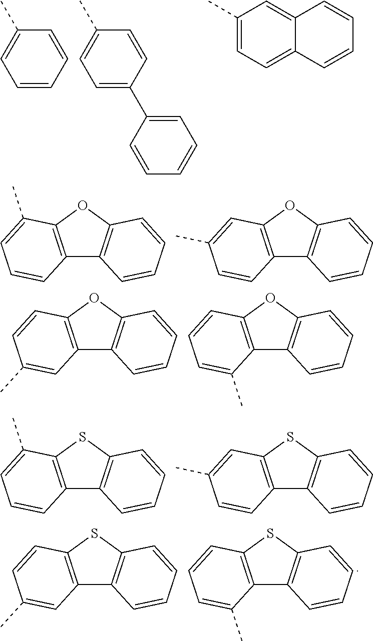

Preferably, n can be 0.

Representative examples of the compound of Chemical Formula 1 are as follows:

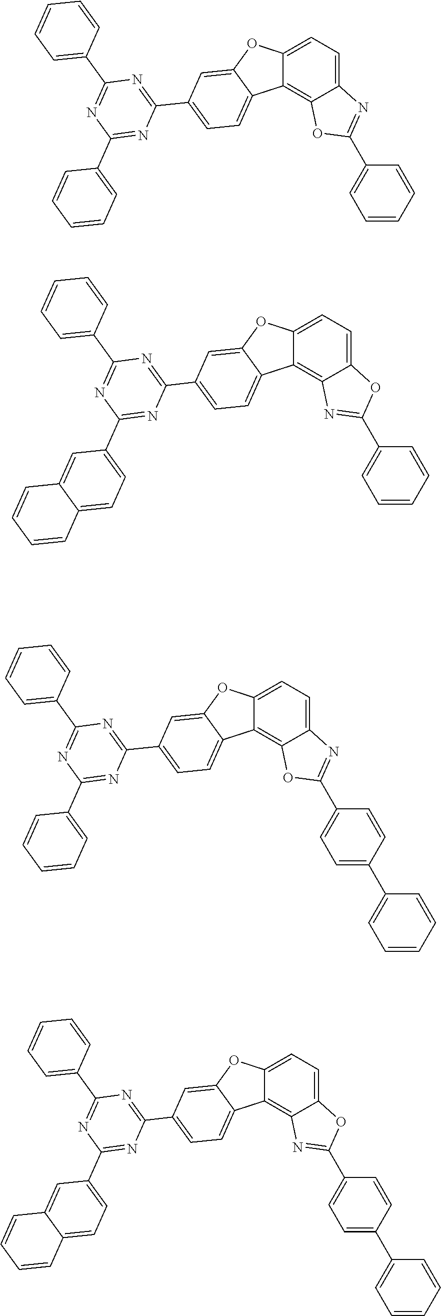 [SMILES: c1ccc(-c2nc(-c3ccccc3)nc(-c3ccc4c(c3)oc3ccc5nc(-c6ccccc6)oc5c34)n2)cc1 ; c1ccc(-c2nc(-c3ccc4ccccc4c3)nc(-c3ccc4c(c3)oc3ccc5oc(-c6ccccc6)nc5c34)n2)cc1 ; c1ccc(-c2ccc(-c3nc4ccc5oc6cc(-c7nc(-c8ccccc8)nc(-c8ccccc8)n7)ccc6c5c4o3)cc2)cc1 ; c1ccc(-c2ccc(-c3nc4c(ccc5oc6cc(-c7nc(-c8ccccc8)nc(-c8ccc9ccccc9c8)n7)ccc6c54)o3)cc2)cc1]

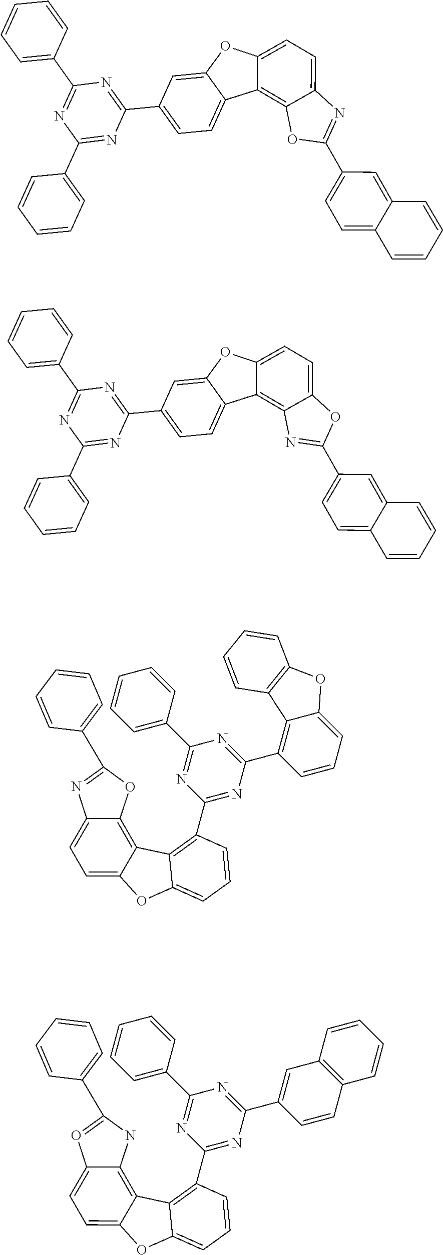

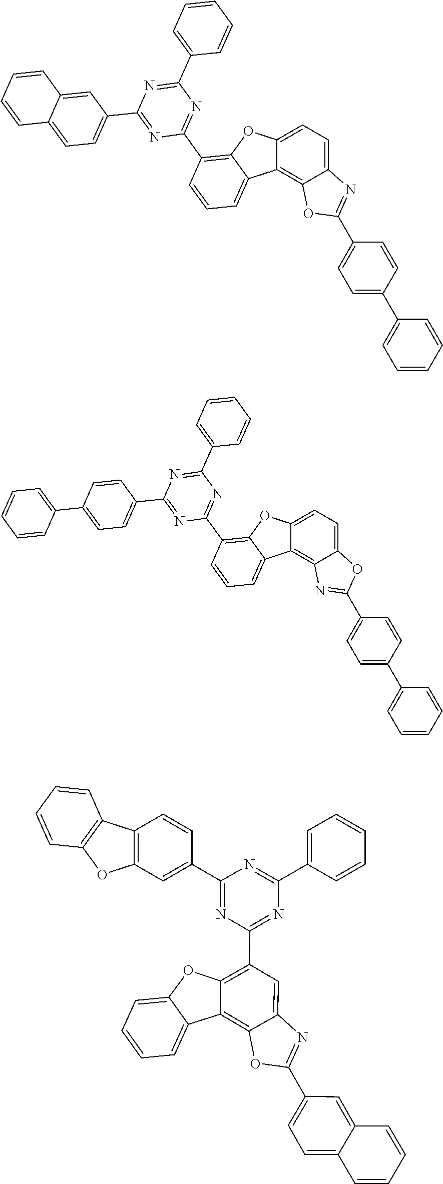 [SMILES: c1ccc(-c2ccc(-c3nc4ccc5oc6c(-c7nc(-c8ccccc8)nc(-c8ccc9ccccc9c8)n7)cccc6c5c4o3)cc2)cc1 ; c1ccc(-c2ccc(-c3nc(-c4ccccc4)nc(-c4cccc5c4oc4ccc6oc(-c7ccc(-c8ccccc8)cc7)nc6c45)n3)cc2)cc1 ; c1ccc(-c2nc(-c3ccc4c(c3)oc3ccccc34)nc(-c3cc4nc(-c5ccc6ccccc6c5)oc4c4c3oc3ccccc34)n2)cc1]

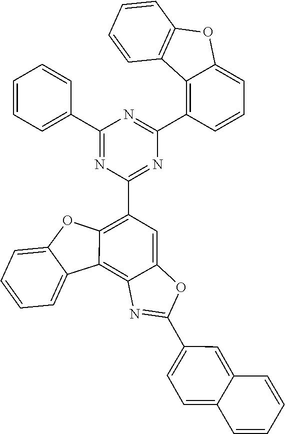 [SMILES: c1ccc(-c2nc(-c3cc4oc(-c5ccc6ccccc6c5)nc4c4c3oc3ccccc34)nc(-c3cccc4oc5ccccc5c34)n2)cc1]

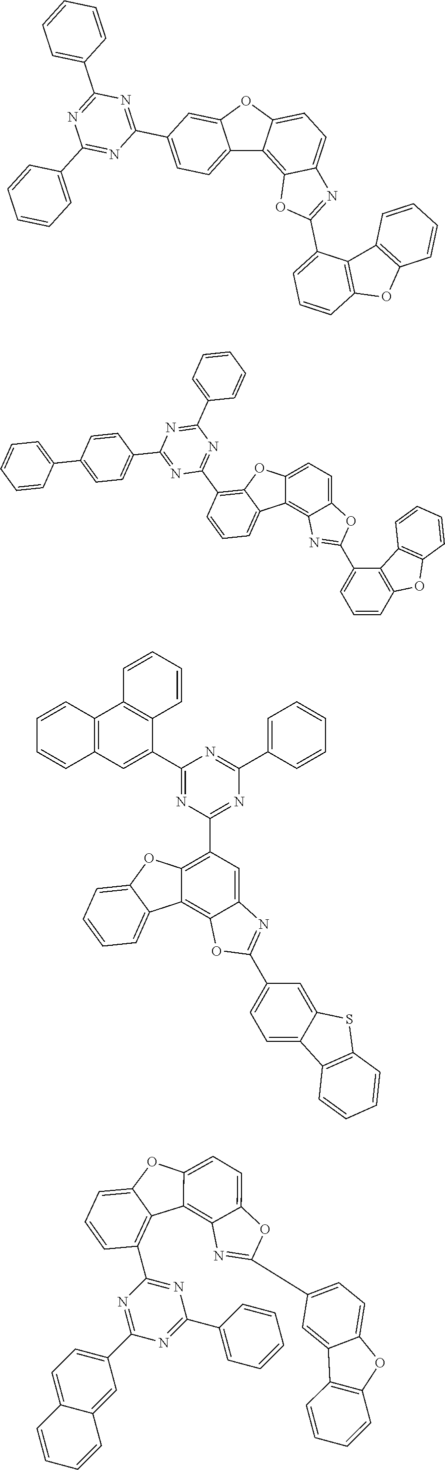 [SMILES: c1ccc(-c2nc(-c3ccccc3)nc(-c3ccc4c(c3)oc3ccc5nc(-c6cccc7oc8ccccc8c67)oc5c34)n2)cc1 ; c1ccc(-c2ccc(-c3nc(-c4ccccc4)nc(-c4cccc5c4oc4ccc6oc(-c7cccc8oc9ccccc9c78)nc6c45)n3)cc2)cc1 ; c1ccc(-c2nc(-c3cc4ccccc4c4ccccc34)nc(-c3cc4nc(-c5ccc6c(c5)sc5ccccc56)oc4c4c3oc3ccccc34)n2)cc1 ; c1ccc(-c2nc(-c3ccc4ccccc4c3)nc(-c3cccc4oc5ccc6oc(-c7ccc8oc9ccccc9c8c7)nc6c5c34)n2)cc1]

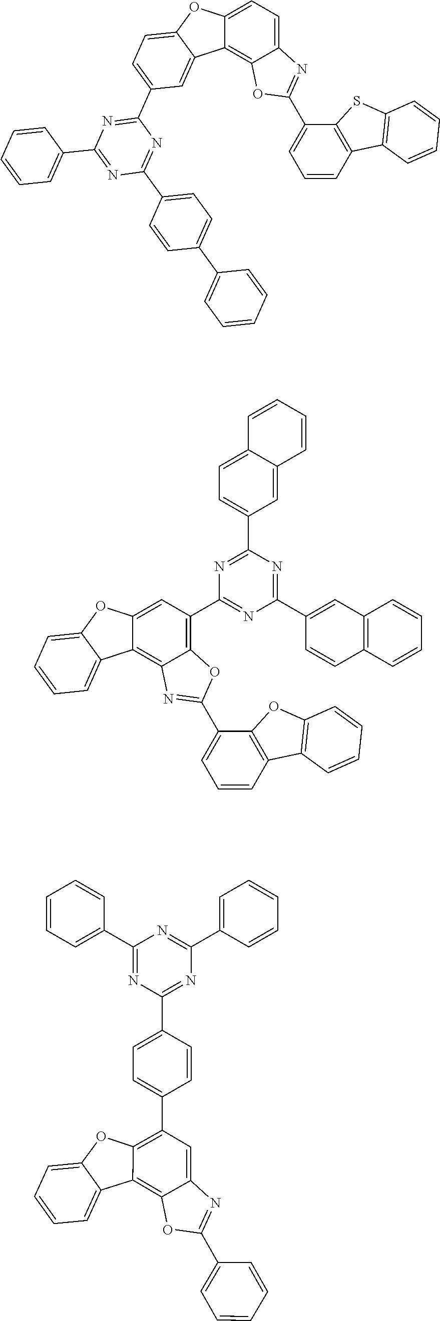 [SMILES: c1ccc(-c2ccc(-c3nc(-c4ccccc4)nc(-c4ccc5oc6ccc7nc(-c8cccc9c8sc8ccccc89)oc7c6c5c4)n3)cc2)cc1 ; c1ccc2cc(-c3nc(-c4ccc5ccccc5c4)nc(-c4cc5oc6ccccc6c5c5nc(-c6cccc7c6oc6ccccc67)oc45)n3)ccc2c1 ; c1ccc(-c2nc(-c3ccccc3)nc(-c3ccc(-c4cc5nc(-c6ccccc6)oc5c5c4oc4ccccc45)cc3)n2)cc1]

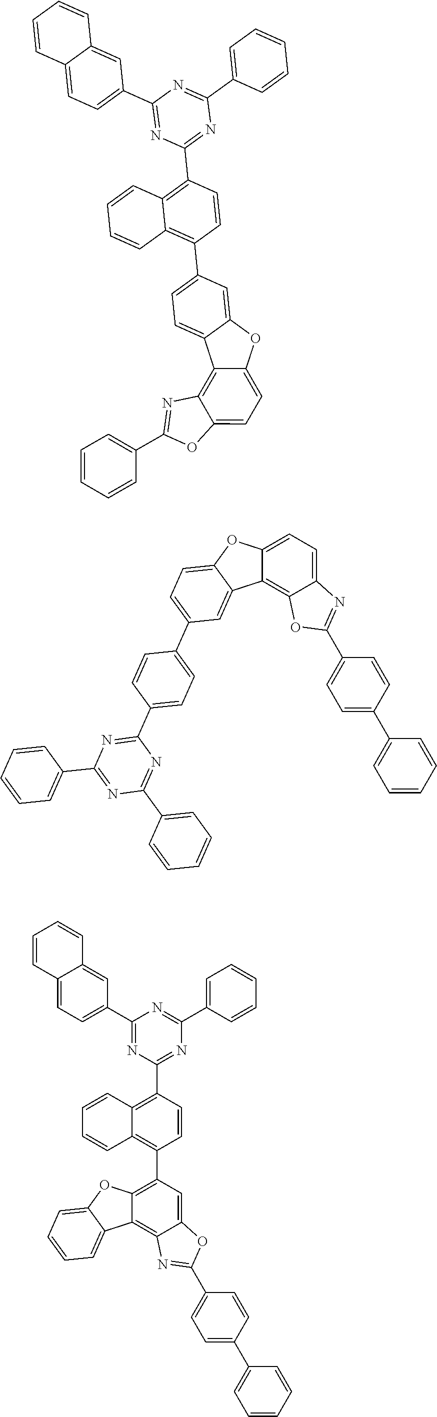 [SMILES: c1ccc(-c2nc(-c3ccc4ccccc4c3)nc(-c3ccc(-c4ccc5c(c4)oc4ccc6oc(-c7ccccc7)nc6c45)c4ccccc34)n2)cc1 ; c1ccc(-c2ccc(-c3nc4ccc5oc6ccc(-c7ccc(-c8nc(-c9ccccc9)nc(-c9ccccc9)n8)cc7)cc6c5c4o3)cc2)cc1 ; c1ccc(-c2ccc(-c3nc4c(cc(-c5ccc(-c6nc(-c7ccccc7)nc(-c7ccc8ccccc8c7)n6)c6ccccc56)c5oc6ccccc6c54)o3)cc2)cc1]

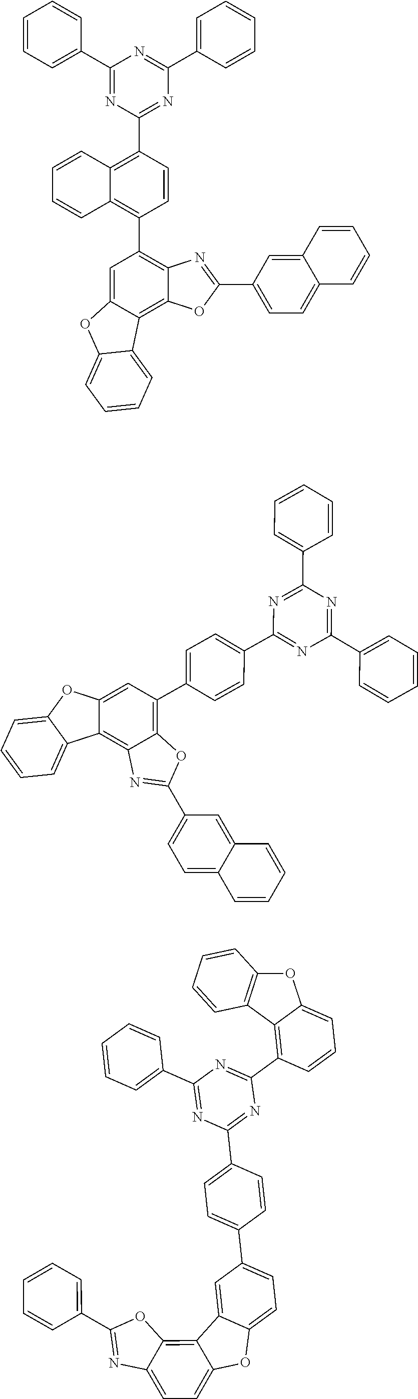 [SMILES: c1ccc(-c2nc(-c3ccccc3)nc(-c3ccc(-c4cc5oc6ccccc6c5c5oc(-c6ccc7ccccc7c6)nc45)c4ccccc34)n2)cc1 ; c1ccc(-c2nc(-c3ccccc3)nc(-c3ccc(-c4cc5oc6ccccc6c5c5nc(-c6ccc7ccccc7c6)oc45)cc3)n2)cc1 ; c1ccc(-c2nc(-c3ccc(-c4ccc5oc6ccc7nc(-c8ccccc8)oc7c6c5c4)cc3)nc(-c3cccc4oc5ccccc5c34)n2)cc1]

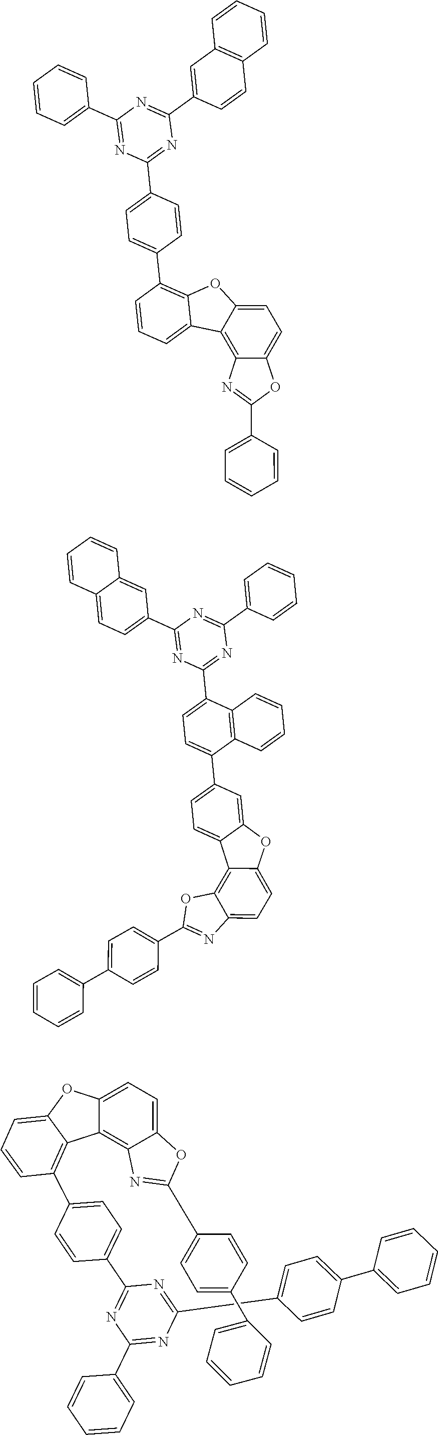 [SMILES: c1ccc(-c2nc(-c3ccc(-c4cccc5c4oc4ccc6oc(-c7ccccc7)nc6c45)cc3)nc(-c3ccc4ccccc4c3)n2)cc1 ; c1ccc(-c2ccc(-c3nc4ccc5oc6cc(-c7ccc(-c8nc(-c9ccccc9)nc(-c9ccc%10ccccc%10c9)n8)c8ccccc78)ccc6c5c4o3)cc2)cc1 ; c1ccc(-c2ccc(-c3nc(-c4ccccc4)nc(-c4ccc(-c5cccc6oc7ccc8oc(-c9ccc(-c%10ccccc%10)cc9)nc8c7c56)cc4)n3)cc2)cc1]

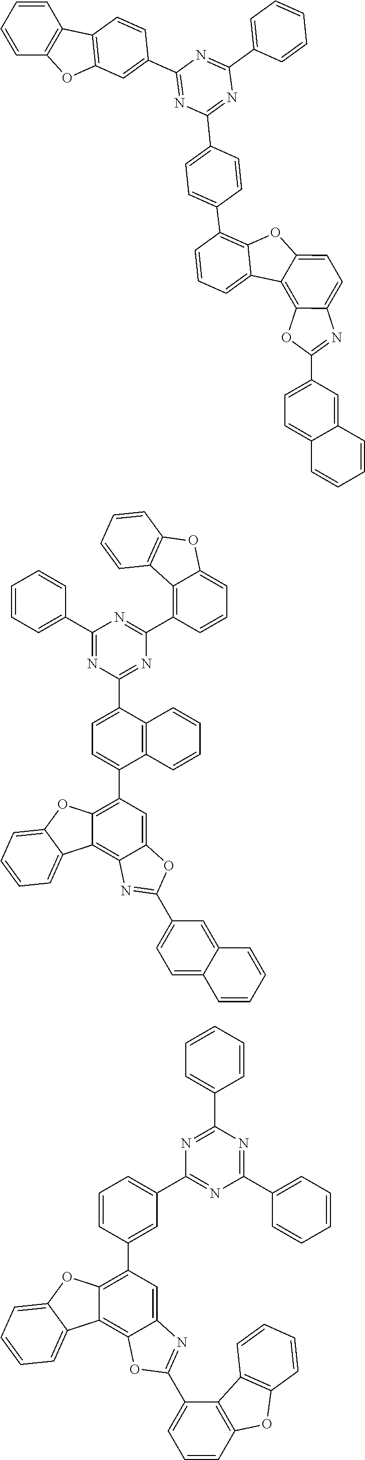

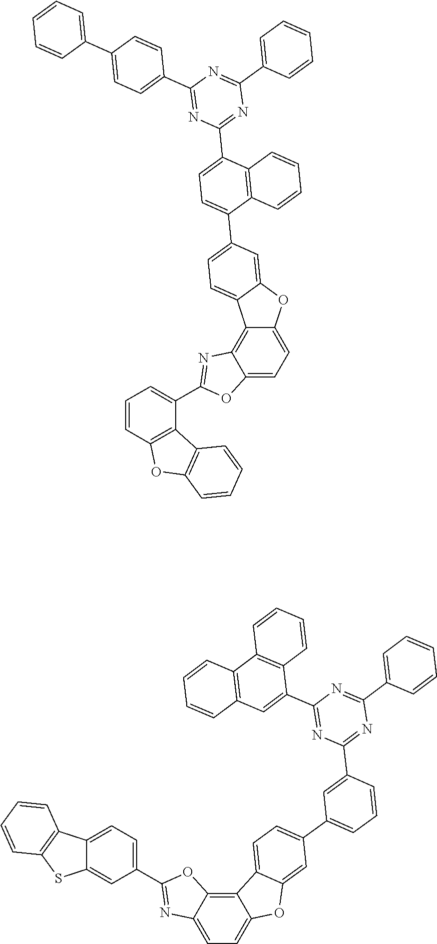 [SMILES: c1ccc(-c2ccc(-c3nc(-c4ccccc4)nc(-c4ccc(-c5ccc6c(c5)oc5ccc7oc(-c8cccc9oc%10ccccc%10c89)nc7c56)c5ccccc45)n3)cc2)cc1 ; c1ccc(-c2nc(-c3cccc(-c4ccc5c(c4)oc4ccc6nc(-c7ccc8c(c7)sc7ccccc78)oc6c45)c3)nc(-c3cc4ccccc4c4ccccc34)n2)cc1]

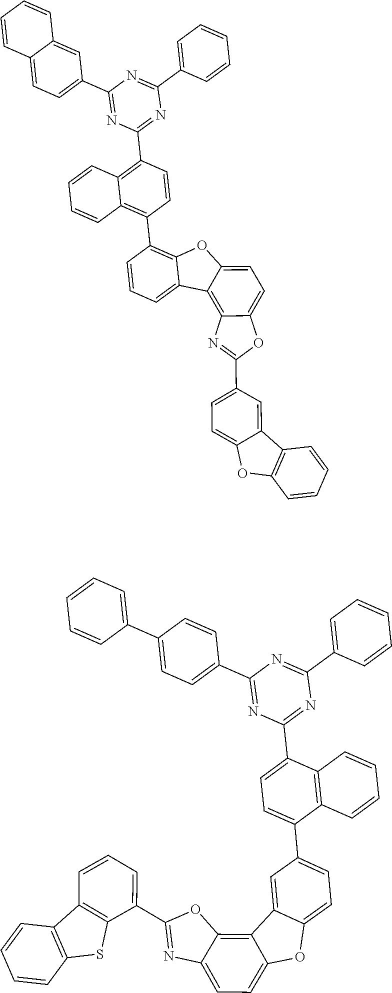 [SMILES: c1ccc(-c2nc(-c3ccc4ccccc4c3)nc(-c3ccc(-c4cccc5c4oc4ccc6oc(-c7ccc8oc9ccccc9c8c7)nc6c45)c4ccccc34)n2)cc1 ; c1ccc(-c2ccc(-c3nc(-c4ccccc4)nc(-c4ccc(-c5ccc6oc7ccc8nc(-c9cccc%10c9sc9ccccc9%10)oc8c7c6c5)c5ccccc45)n3)cc2)cc1]

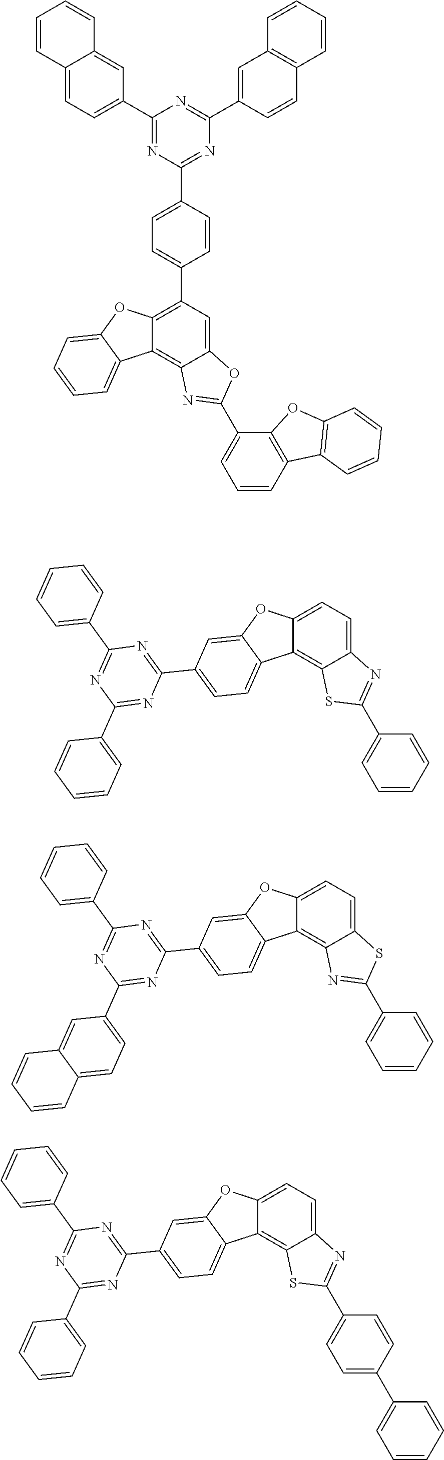

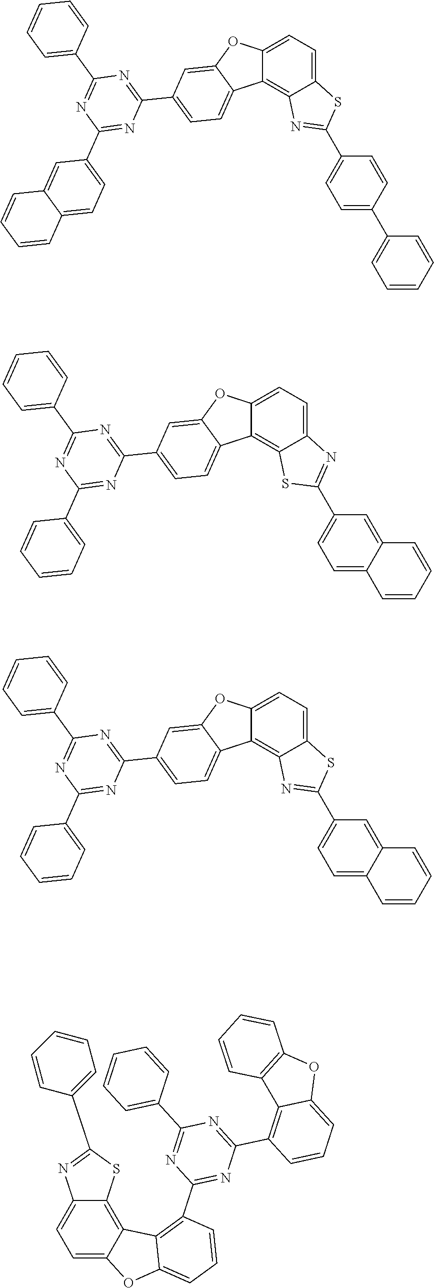 [SMILES: c1ccc(-c2ccc(-c3nc4c(ccc5oc6cc(-c7nc(-c8ccccc8)nc(-c8ccc9ccccc9c8)n7)ccc6c54)s3)cc2)cc1 ; c1ccc(-c2nc(-c3ccccc3)nc(-c3ccc4c(c3)oc3ccc5nc(-c6ccc7ccccc7c6)sc5c34)n2)cc1 ; c1ccc(-c2nc(-c3ccccc3)nc(-c3ccc4c(c3)oc3ccc5sc(-c6ccc7ccccc7c6)nc5c34)n2)cc1 ; c1ccc(-c2nc(-c3cccc4oc5ccccc5c34)nc(-c3cccc4oc5ccc6nc(-c7ccccc7)sc6c5c34)n2)cc1]

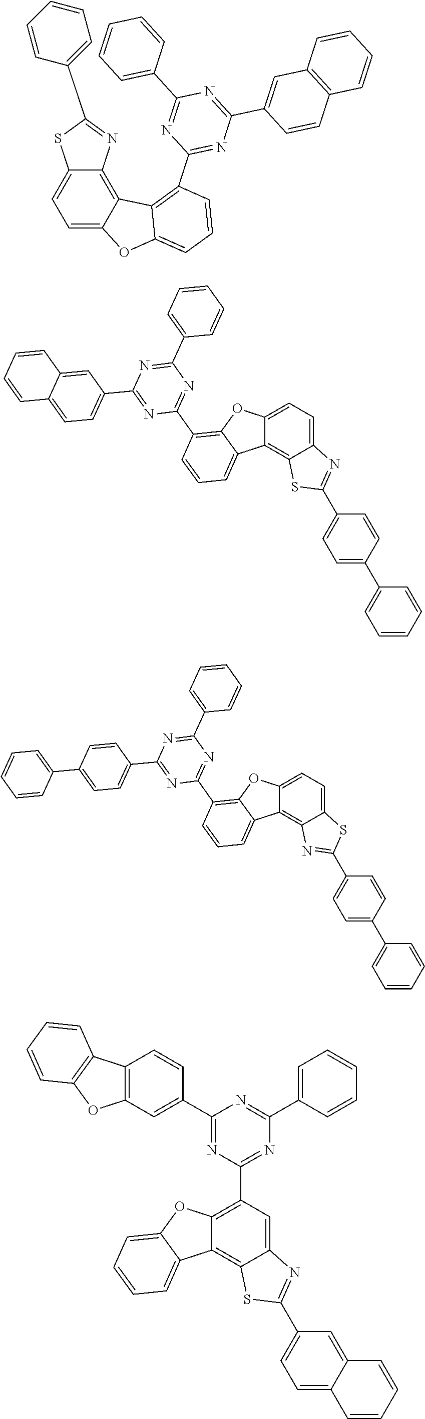

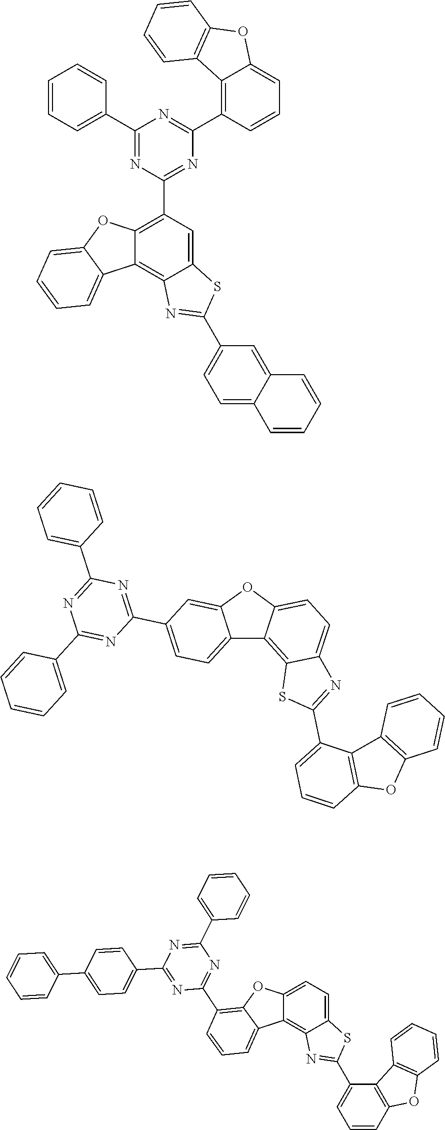 [SMILES: c1ccc(-c2nc(-c3cc4sc(-c5ccc6ccccc6c5)nc4c4c3oc3ccccc34)nc(-c3cccc4oc5ccccc5c34)n2)cc1 ; c1ccc(-c2nc(-c3ccccc3)nc(-c3ccc4c(c3)oc3ccc5nc(-c6cccc7oc8ccccc8c67)sc5c34)n2)cc1 ; c1ccc(-c2ccc(-c3nc(-c4ccccc4)nc(-c4cccc5c4oc4ccc6sc(-c7cccc8oc9ccccc9c78)nc6c45)n3)cc2)cc1]

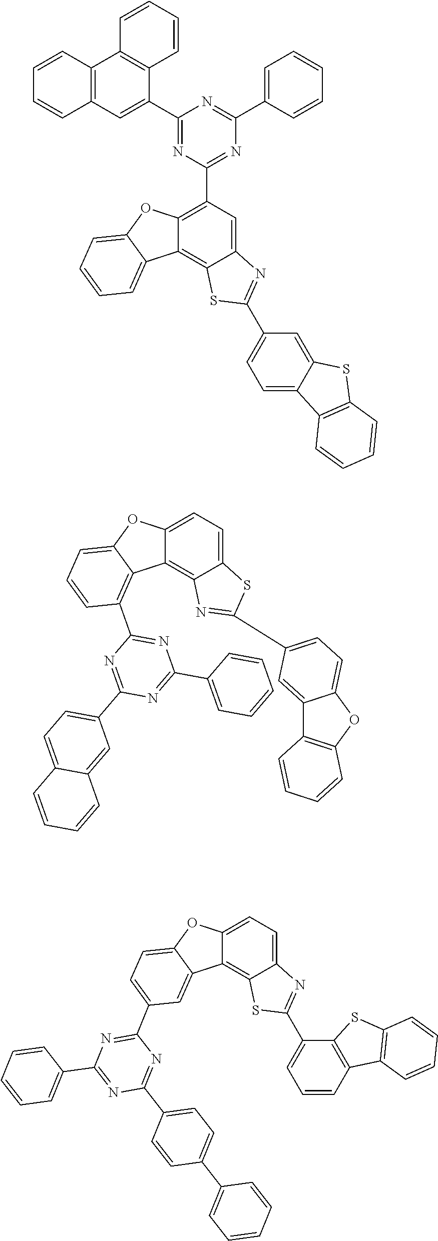 [SMILES: c1ccc(-c2nc(-c3cc4ccccc4c4ccccc34)nc(-c3cc4nc(-c5ccc6c(c5)sc5ccccc56)sc4c4c3oc3ccccc34)n2)cc1 ; c1ccc(-c2nc(-c3ccc4ccccc4c3)nc(-c3cccc4oc5ccc6sc(-c7ccc8oc9ccccc9c8c7)nc6c5c34)n2)cc1 ; c1ccc(-c2ccc(-c3nc(-c4ccccc4)nc(-c4ccc5oc6ccc7nc(-c8cccc9c8sc8ccccc89)sc7c6c5c4)n3)cc2)cc1]

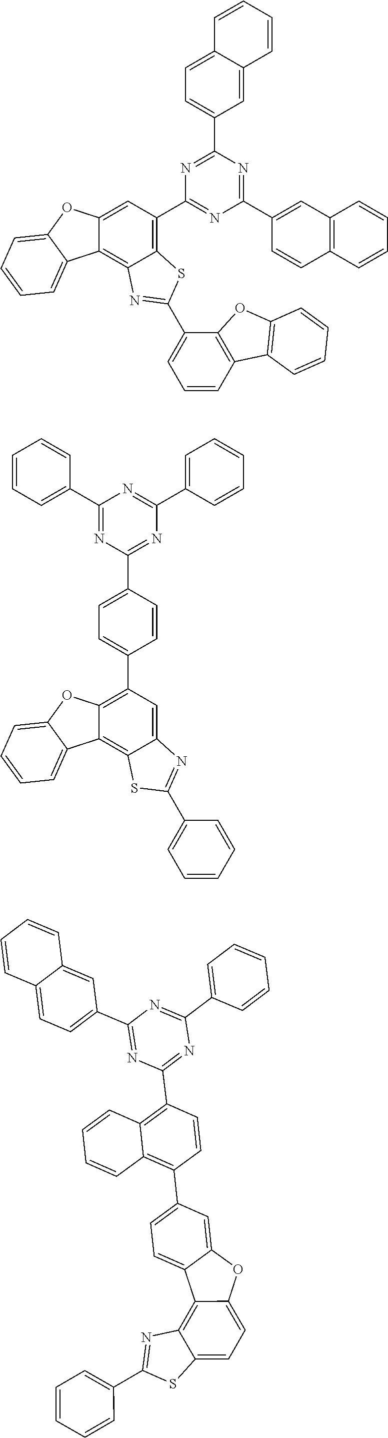 [SMILES: c1ccc2cc(-c3nc(-c4ccc5ccccc5c4)nc(-c4cc5oc6ccccc6c5c5nc(-c6cccc7c6oc6ccccc67)sc45)n3)ccc2c1 ; c1ccc(-c2nc(-c3ccccc3)nc(-c3ccc(-c4cc5nc(-c6ccccc6)sc5c5c4oc4ccccc45)cc3)n2)cc1 ; c1ccc(-c2nc(-c3ccc4ccccc4c3)nc(-c3ccc(-c4ccc5c(c4)oc4ccc6sc(-c7ccccc7)nc6c45)c4ccccc34)n2)cc1]

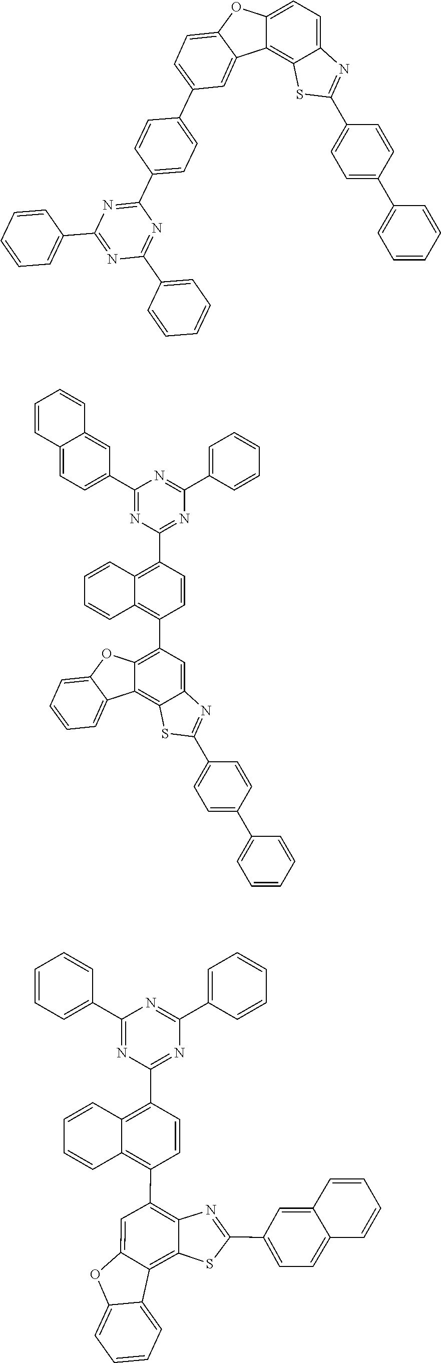 [SMILES: c1ccc(-c2ccc(-c3nc4ccc5oc6ccc(-c7ccc(-c8nc(-c9ccccc9)nc(-c9ccccc9)n8)cc7)cc6c5c4s3)cc2)cc1 ; c1ccc(-c2ccc(-c3nc4cc(-c5ccc(-c6nc(-c7ccccc7)nc(-c7ccc8ccccc8c7)n6)c6ccccc56)c5oc6ccccc6c5c4s3)cc2)cc1 ; c1ccc(-c2nc(-c3ccccc3)nc(-c3ccc(-c4cc5oc6ccccc6c5c5sc(-c6ccc7ccccc7c6)nc45)c4ccccc34)n2)cc1]

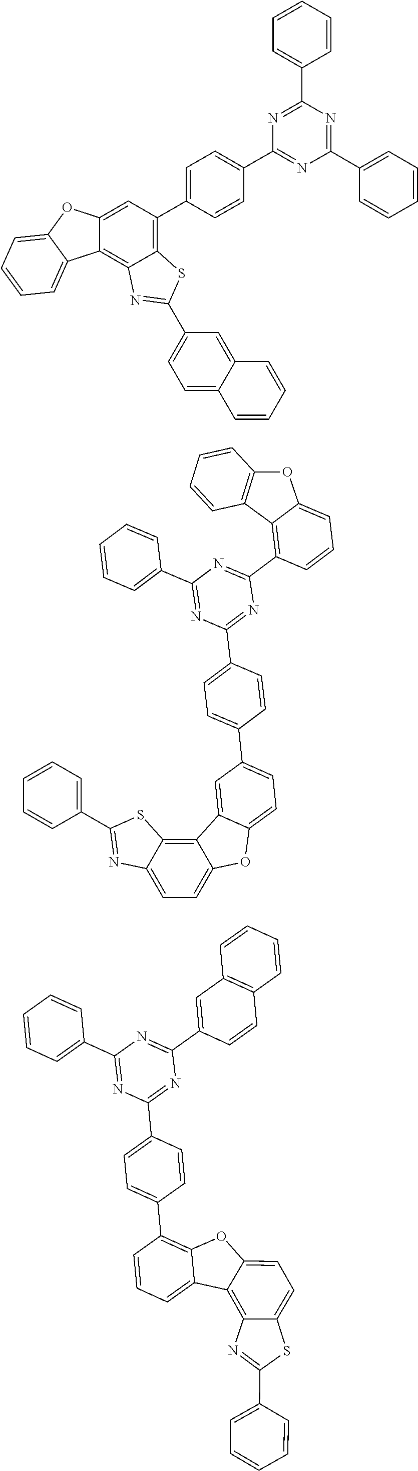

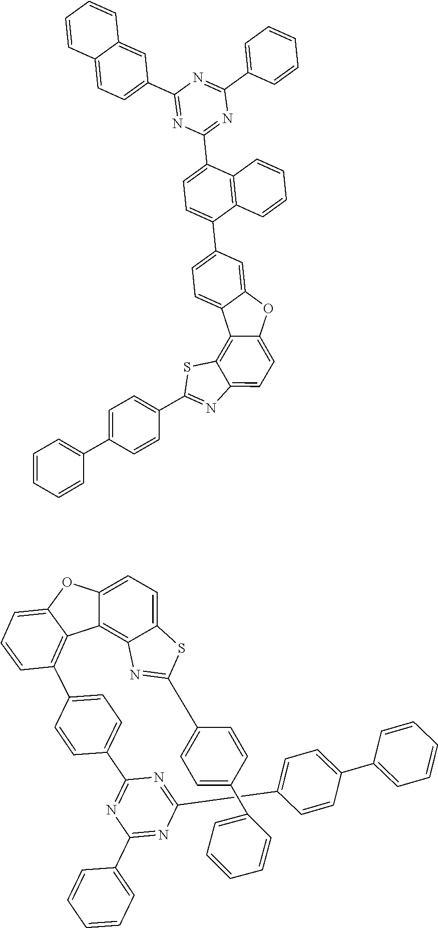 [SMILES: c1ccc(-c2ccc(-c3nc4ccc5oc6cc(-c7ccc(-c8nc(-c9ccccc9)nc(-c9ccc%10ccccc%10c9)n8)c8ccccc78)ccc6c5c4s3)cc2)cc1 ; c1ccc(-c2ccc(-c3nc(-c4ccccc4)nc(-c4ccc(-c5cccc6oc7ccc8sc(-c9ccc(-c%10ccccc%10)cc9)nc8c7c56)cc4)n3)cc2)cc1]

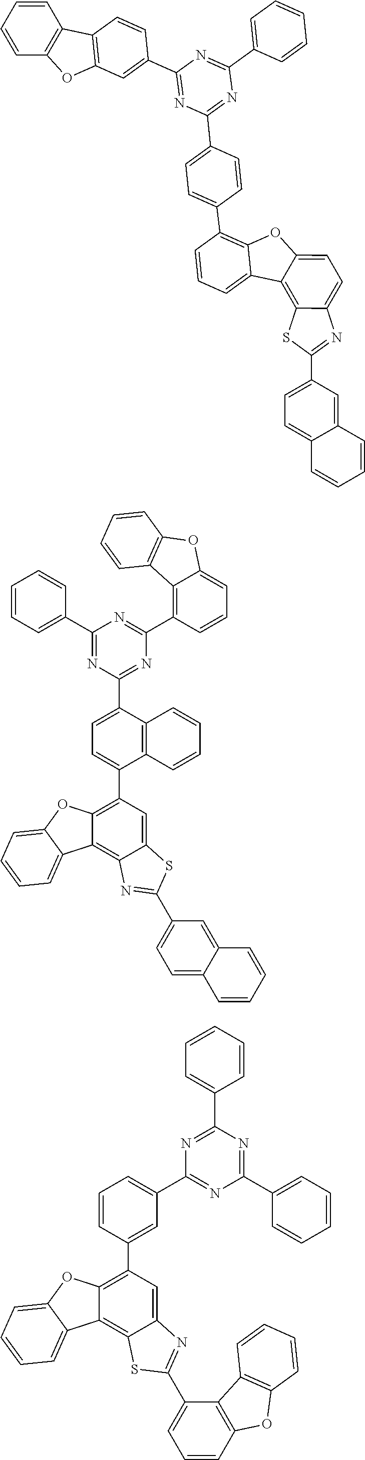

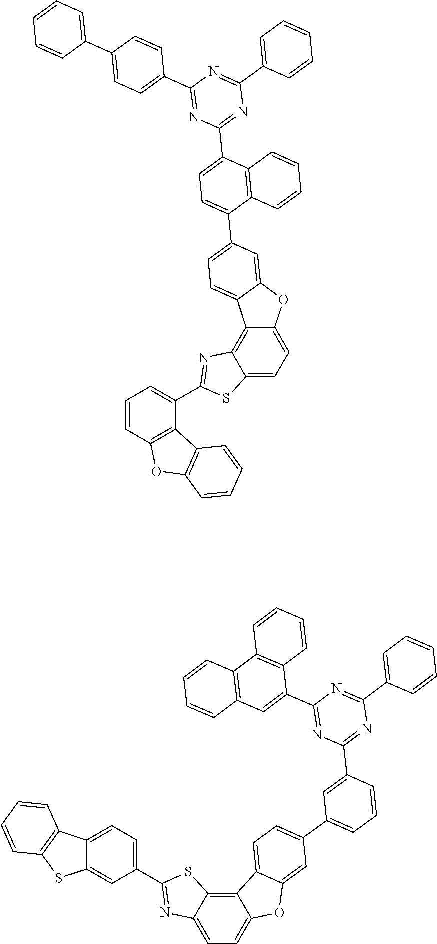 [SMILES: c1ccc(-c2ccc(-c3nc(-c4ccccc4)nc(-c4ccc(-c5ccc6c(c5)oc5ccc7sc(-c8cccc9oc%10ccccc%10c89)nc7c56)c5ccccc45)n3)cc2)cc1 ; c1ccc(-c2nc(-c3cccc(-c4ccc5c(c4)oc4ccc6nc(-c7ccc8c(c7)sc7ccccc78)sc6c45)c3)nc(-c3cc4ccccc4c4ccccc34)n2)cc1]

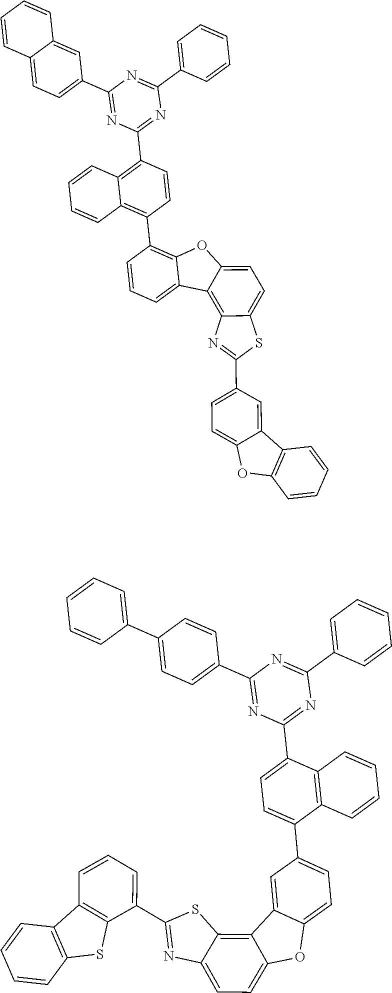 [SMILES: c1ccc(-c2nc(-c3ccc4ccccc4c3)nc(-c3ccc(-c4cccc5c4oc4ccc6sc(-c7ccc8oc9ccccc9c8c7)nc6c45)c4ccccc34)n2)cc1 ; c1ccc(-c2ccc(-c3nc(-c4ccccc4)nc(-c4ccc(-c5ccc6oc7ccc8nc(-c9cccc%10c9sc9ccccc9%10)sc8c7c6c5)c5ccccc45)n3)cc2)cc1]

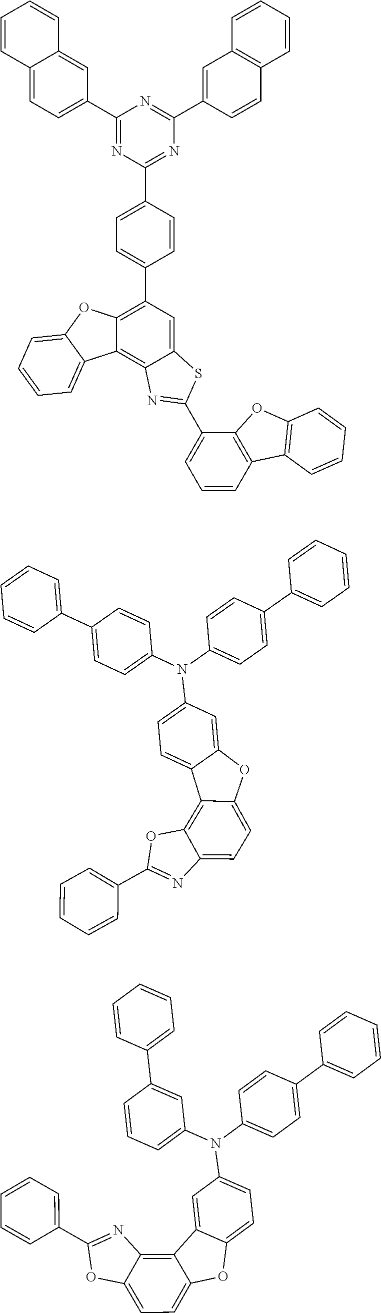 [SMILES: c1ccc2cc(-c3nc(-c4ccc(-c5cc6sc(-c7cccc8c7oc7ccccc78)nc6c6c5oc5ccccc56)cc4)nc(-c4ccc5ccccc5c4)n3)ccc2c1 ; c1ccc(-c2ccc(N(c3ccc(-c4ccccc4)cc3)c3ccc4c(c3)oc3ccc5nc(-c6ccccc6)oc5c34)cc2)cc1 ; c1ccc(-c2ccc(N(c3cccc(-c4ccccc4)c3)c3ccc4oc5ccc6oc(-c7ccccc7)nc6c5c4c3)cc2)cc1]

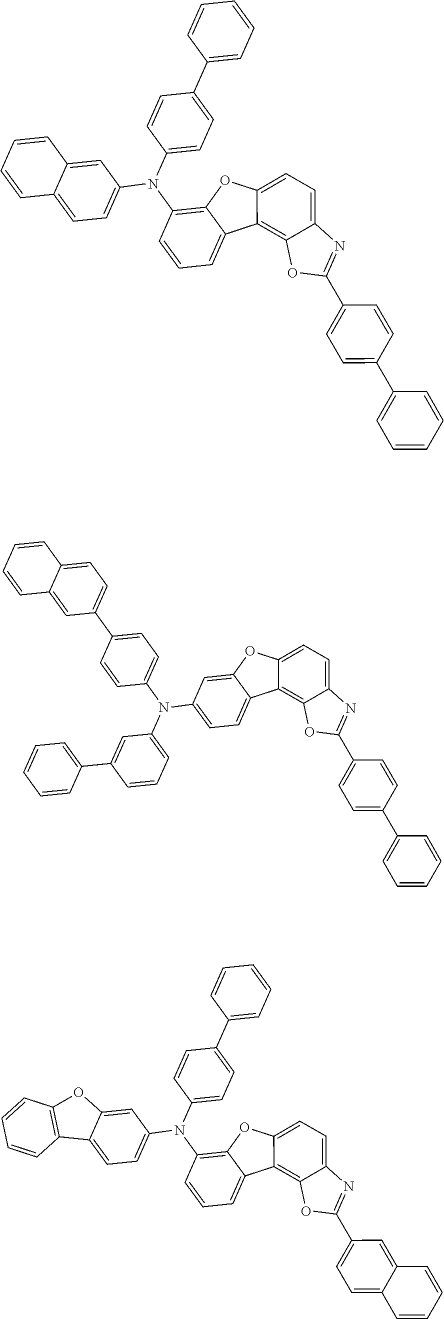 [SMILES: c1ccc(-c2ccc(-c3nc4ccc5oc6c(N(c7ccc(-c8ccccc8)cc7)c7ccc8ccccc8c7)cccc6c5c4o3)cc2)cc1 ; c1ccc(-c2ccc(-c3nc4ccc5oc6cc(N(c7ccc(-c8ccc9ccccc9c8)cc7)c7cccc(-c8ccccc8)c7)ccc6c5c4o3)cc2)cc1 ; c1ccc(-c2ccc(N(c3ccc4c(c3)oc3ccccc34)c3cccc4c3oc3ccc5nc(-c6ccc7ccccc7c6)oc5c34)cc2)cc1]

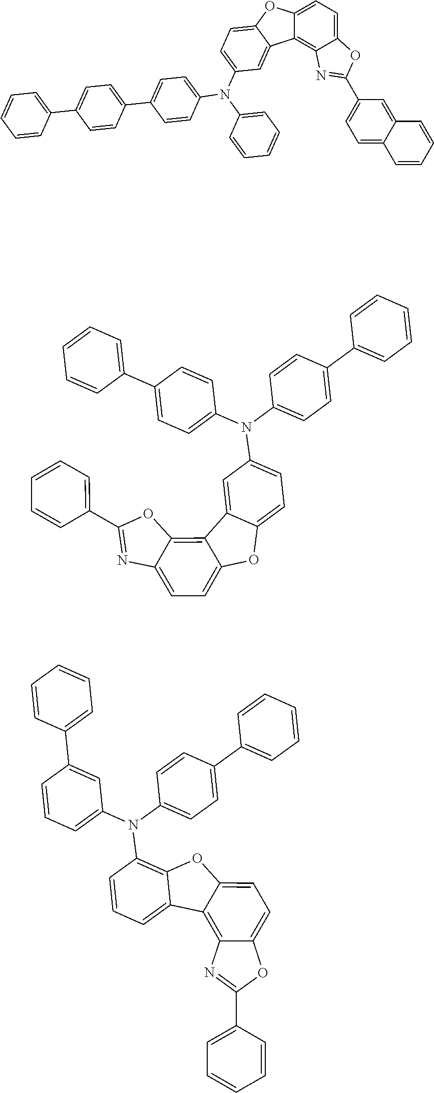 [SMILES: c1ccc(-c2ccc(-c3ccc(N(c4ccccc4)c4ccc5oc6ccc7oc(-c8ccc9ccccc9c8)nc7c6c5c4)cc3)cc2)cc1 ; c1ccc(-c2ccc(N(c3ccc(-c4ccccc4)cc3)c3ccc4oc5ccc6nc(-c7ccccc7)oc6c5c4c3)cc2)cc1 ; c1ccc(-c2ccc(N(c3cccc(-c4ccccc4)c3)c3cccc4c3oc3ccc5oc(-c6ccccc6)nc5c34)cc2)cc1]

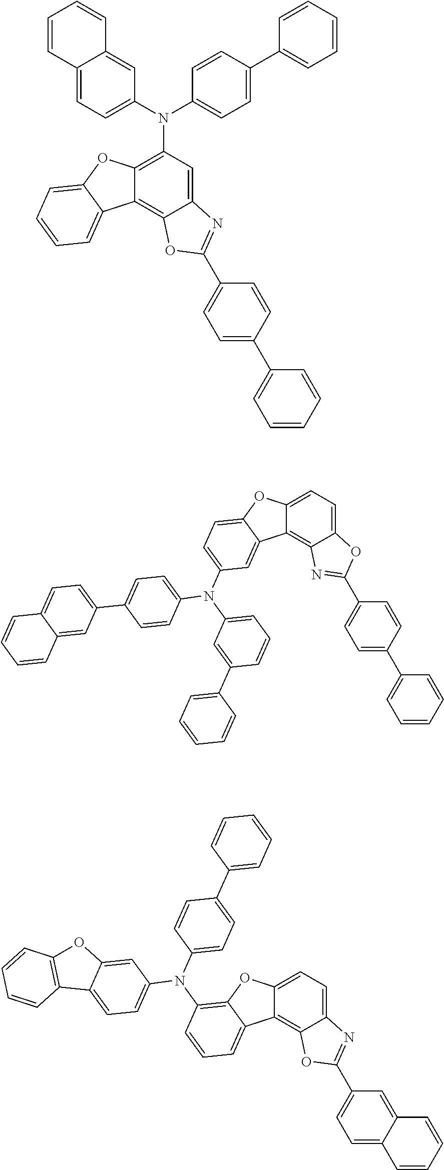 [SMILES: c1ccc(-c2ccc(-c3nc4cc(N(c5ccc(-c6ccccc6)cc5)c5ccc6ccccc6c5)c5oc6ccccc6c5c4o3)cc2)cc1 ; c1ccc(-c2ccc(-c3nc4c(ccc5oc6ccc(N(c7ccc(-c8ccc9ccccc9c8)cc7)c7cccc(-c8ccccc8)c7)cc6c54)o3)cc2)cc1 ; c1ccc(-c2ccc(N(c3ccc4c(c3)oc3ccccc34)c3cccc4c3oc3ccc5nc(-c6ccc7ccccc7c6)oc5c34)cc2)cc1]

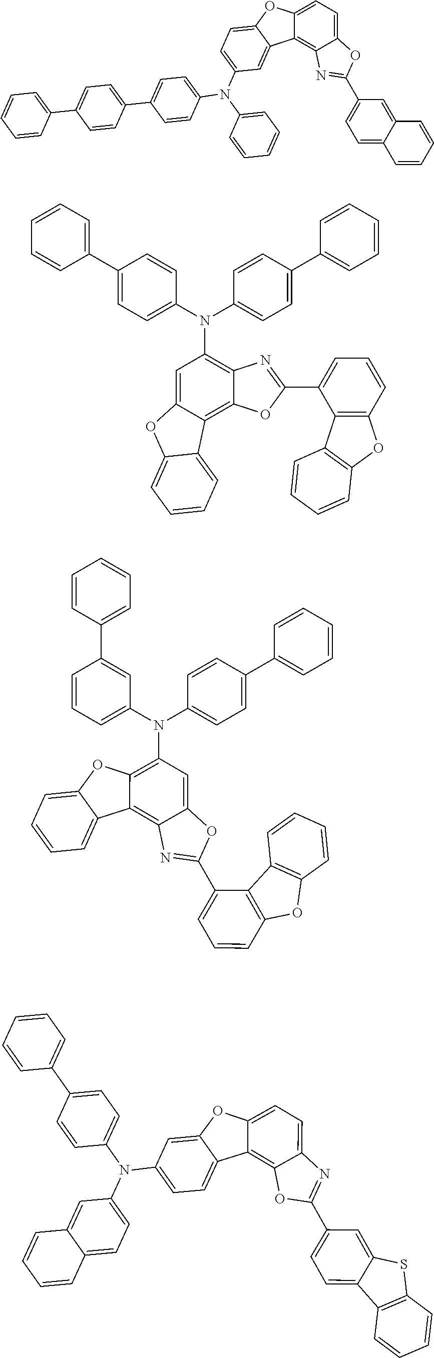 [SMILES: c1ccc(-c2ccc(-c3ccc(N(c4ccccc4)c4ccc5oc6ccc7oc(-c8ccc9ccccc9c8)nc7c6c5c4)cc3)cc2)cc1 ; c1ccc(-c2ccc(N(c3ccc(-c4ccccc4)cc3)c3cc4oc5ccccc5c4c4oc(-c5cccc6oc7ccccc7c56)nc34)cc2)cc1 ; c1ccc(-c2ccc(N(c3cccc(-c4ccccc4)c3)c3cc4oc(-c5cccc6oc7ccccc7c56)nc4c4c3oc3ccccc34)cc2)cc1 ; c1ccc(-c2ccc(N(c3ccc4ccccc4c3)c3ccc4c(c3)oc3ccc5nc(-c6ccc7c(c6)sc6ccccc67)oc5c34)cc2)cc1]

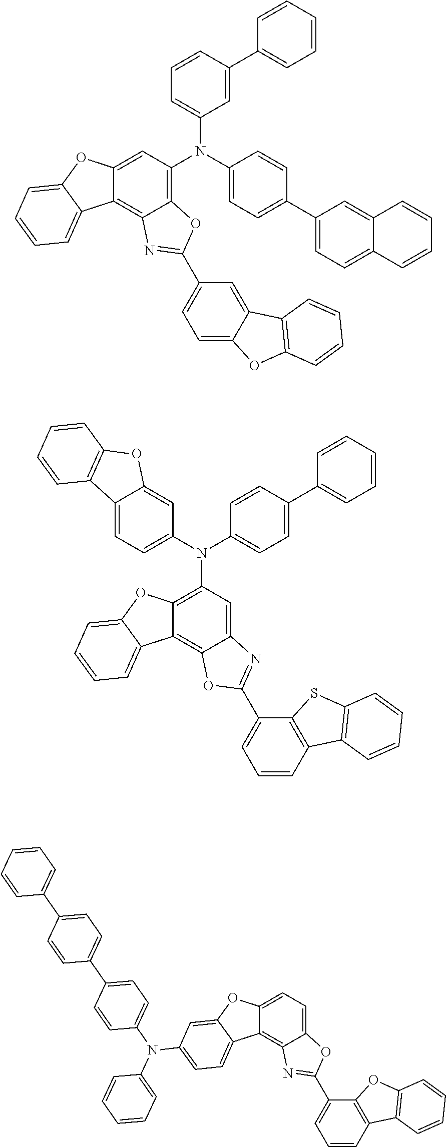 [SMILES: c1ccc(-c2cccc(N(c3ccc(-c4ccc5ccccc5c4)cc3)c3cc4oc5ccccc5c4c4nc(-c5ccc6oc7ccccc7c6c5)oc34)c2)cc1 ; c1ccc(-c2ccc(N(c3ccc4c(c3)oc3ccccc34)c3cc4nc(-c5cccc6c5sc5ccccc56)oc4c4c3oc3ccccc34)cc2)cc1 ; c1ccc(-c2ccc(-c3ccc(N(c4ccccc4)c4ccc5c(c4)oc4ccc6oc(-c7cccc8c7oc7ccccc78)nc6c45)cc3)cc2)cc1]

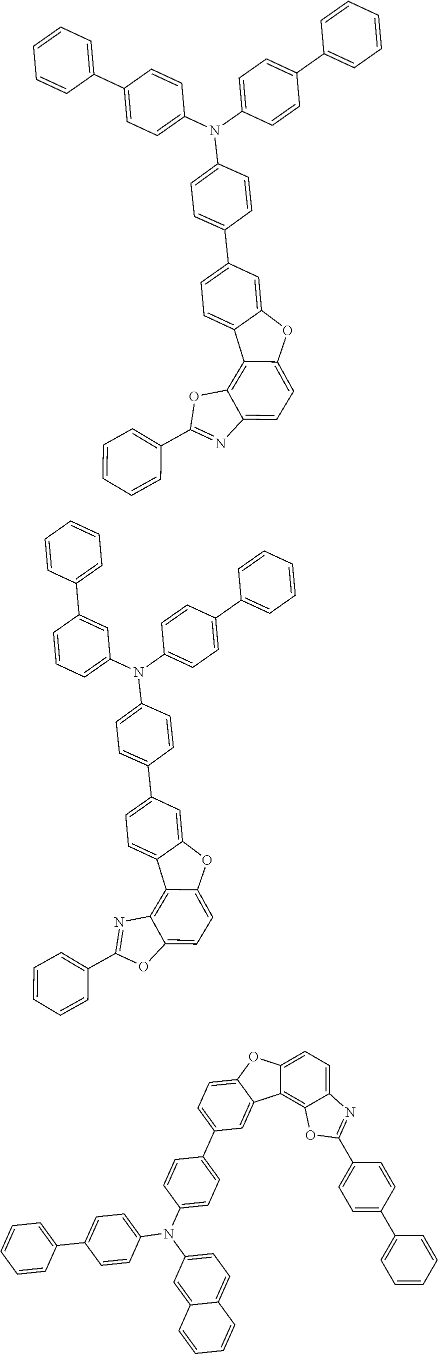 [SMILES: c1ccc(-c2ccc(N(c3ccc(-c4ccccc4)cc3)c3ccc(-c4ccc5c(c4)oc4ccc6nc(-c7ccccc7)oc6c45)cc3)cc2)cc1 ; c1ccc(-c2ccc(N(c3ccc(-c4ccc5c(c4)oc4ccc6oc(-c7ccccc7)nc6c45)cc3)c3cccc(-c4ccccc4)c3)cc2)cc1 ; c1ccc(-c2ccc(-c3nc4ccc5oc6ccc(-c7ccc(N(c8ccc(-c9ccccc9)cc8)c8ccc9ccccc9c8)cc7)cc6c5c4o3)cc2)cc1]

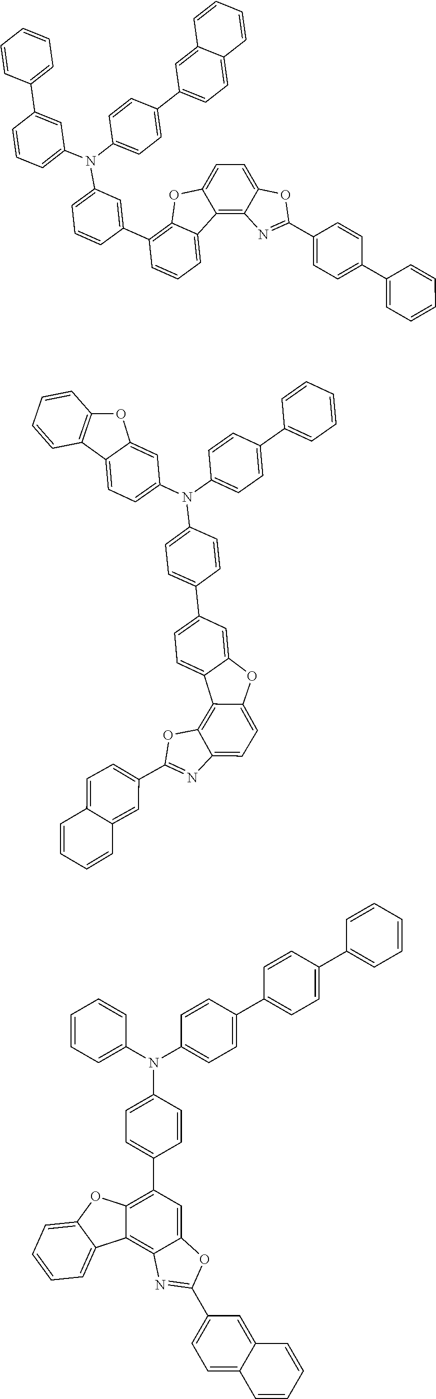 [SMILES: c1ccc(-c2ccc(-c3nc4c(ccc5oc6c(-c7cccc(N(c8ccc(-c9ccc%10ccccc%10c9)cc8)c8cccc(-c9ccccc9)c8)c7)cccc6c54)o3)cc2)cc1 ; c1ccc(-c2ccc(N(c3ccc(-c4ccc5c(c4)oc4ccc6nc(-c7ccc8ccccc8c7)oc6c45)cc3)c3ccc4c(c3)oc3ccccc34)cc2)cc1 ; c1ccc(-c2ccc(-c3ccc(N(c4ccccc4)c4ccc(-c5cc6oc(-c7ccc8ccccc8c7)nc6c6c5oc5ccccc56)cc4)cc3)cc2)cc1]

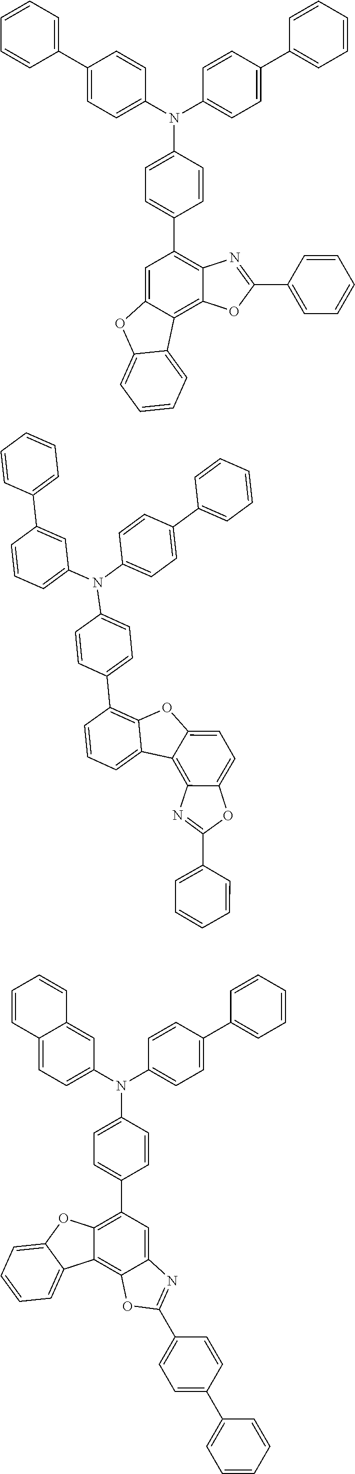 [SMILES: c1ccc(-c2ccc(N(c3ccc(-c4ccccc4)cc3)c3ccc(-c4cc5oc6ccccc6c5c5oc(-c6ccccc6)nc45)cc3)cc2)cc1 ; c1ccc(-c2ccc(N(c3ccc(-c4cccc5c4oc4ccc6oc(-c7ccccc7)nc6c45)cc3)c3cccc(-c4ccccc4)c3)cc2)cc1 ; c1ccc(-c2ccc(-c3nc4cc(-c5ccc(N(c6ccc(-c7ccccc7)cc6)c6ccc7ccccc7c6)cc5)c5oc6ccccc6c5c4o3)cc2)cc1]

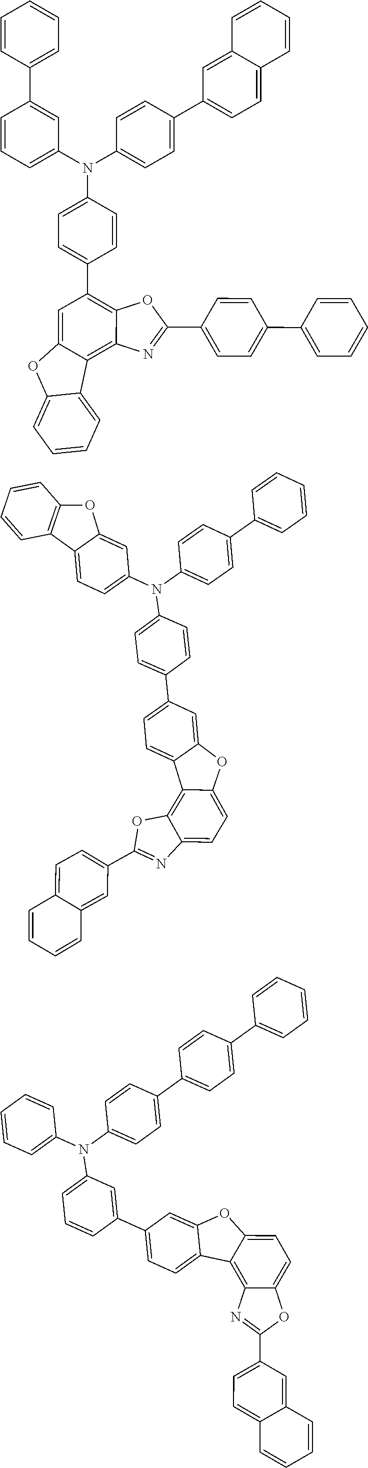

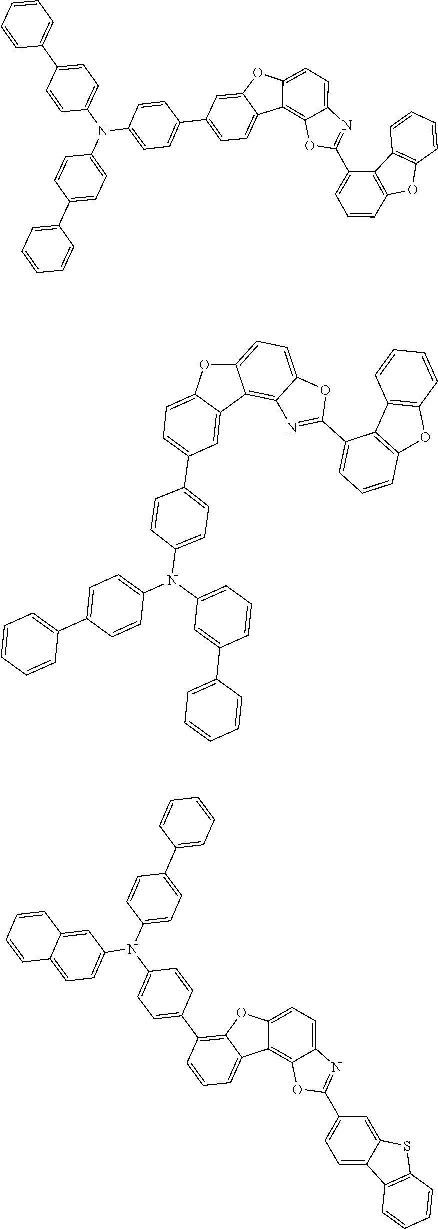 [SMILES: c1ccc(-c2ccc(N(c3ccc(-c4ccccc4)cc3)c3ccc(-c4ccc5c(c4)oc4ccc6nc(-c7cccc8oc9ccccc9c78)oc6c45)cc3)cc2)cc1 ; c1ccc(-c2ccc(N(c3ccc(-c4ccc5oc6ccc7oc(-c8cccc9oc%10ccccc%10c89)nc7c6c5c4)cc3)c3cccc(-c4ccccc4)c3)cc2)cc1 ; c1ccc(-c2ccc(N(c3ccc(-c4cccc5c4oc4ccc6nc(-c7ccc8c(c7)sc7ccccc78)oc6c45)cc3)c3ccc4ccccc4c3)cc2)cc1]

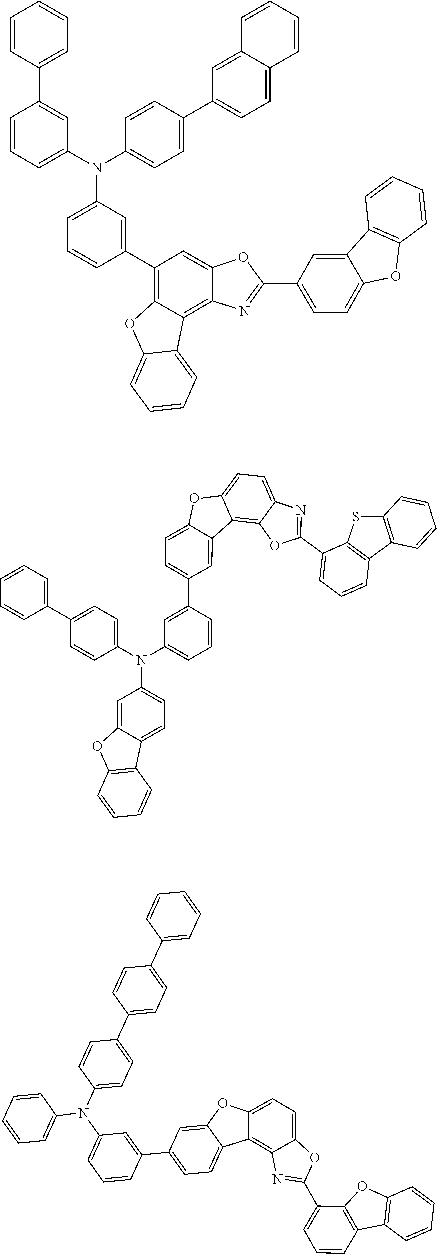 [SMILES: c1ccc(-c2cccc(N(c3ccc(-c4ccc5ccccc5c4)cc3)c3cccc(-c4cc5oc(-c6ccc7oc8ccccc8c7c6)nc5c5c4oc4ccccc45)c3)c2)cc1 ; c1ccc(-c2ccc(N(c3cccc(-c4ccc5oc6ccc7nc(-c8cccc9c8sc8ccccc89)oc7c6c5c4)c3)c3ccc4c(c3)oc3ccccc34)cc2)cc1 ; c1ccc(-c2ccc(-c3ccc(N(c4ccccc4)c4cccc(-c5ccc6c(c5)oc5ccc7oc(-c8cccc9c8oc8ccccc89)nc7c56)c4)cc3)cc2)cc1]

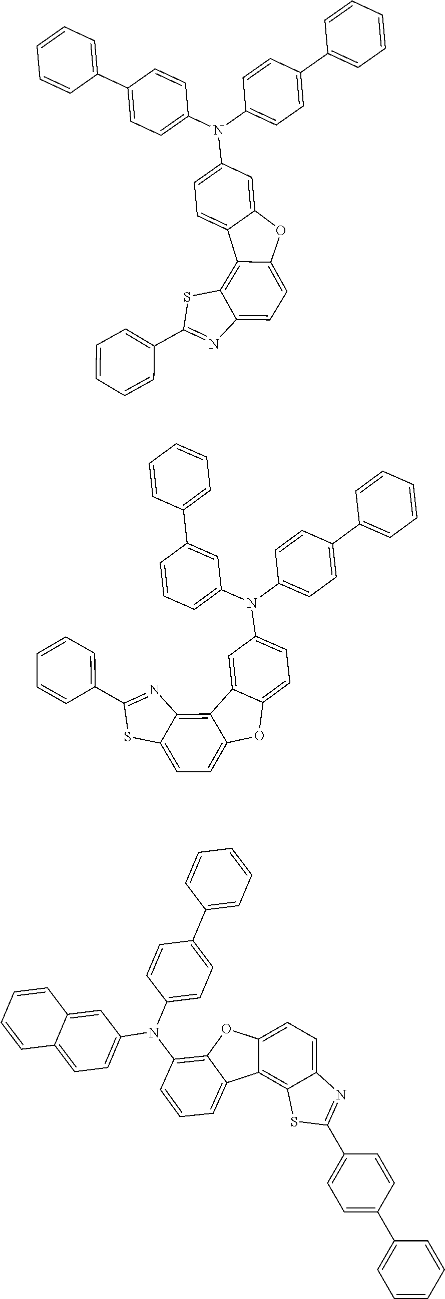 [SMILES: c1ccc(-c2ccc(N(c3ccc(-c4ccccc4)cc3)c3ccc4c(c3)oc3ccc5nc(-c6ccccc6)sc5c34)cc2)cc1 ; c1ccc(-c2ccc(N(c3cccc(-c4ccccc4)c3)c3ccc4oc5ccc6sc(-c7ccccc7)nc6c5c4c3)cc2)cc1 ; c1ccc(-c2ccc(-c3nc4ccc5oc6c(N(c7ccc(-c8ccccc8)cc7)c7ccc8ccccc8c7)cccc6c5c4s3)cc2)cc1]

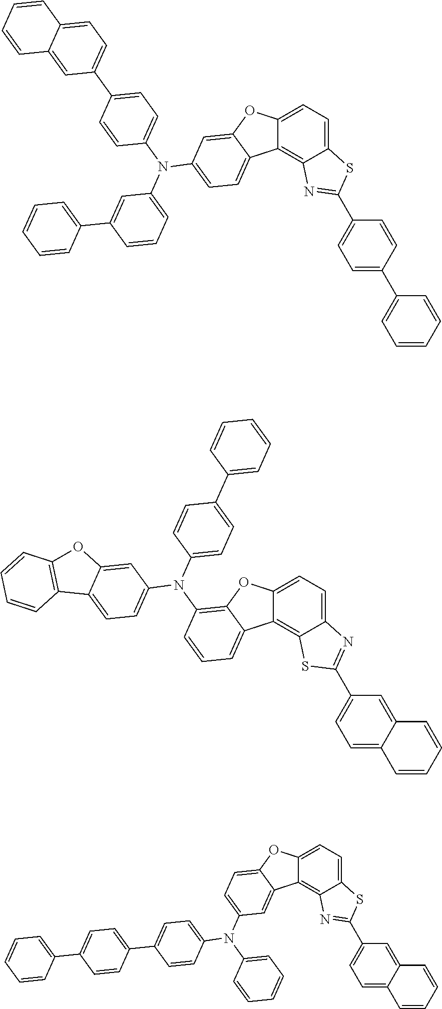 [SMILES: c1ccc(-c2ccc(-c3nc4c(ccc5oc6cc(N(c7ccc(-c8ccc9ccccc9c8)cc7)c7cccc(-c8ccccc8)c7)ccc6c54)s3)cc2)cc1 ; c1ccc(-c2ccc(N(c3ccc4c(c3)oc3ccccc34)c3cccc4c3oc3ccc5nc(-c6ccc7ccccc7c6)sc5c34)cc2)cc1 ; c1ccc(-c2ccc(-c3ccc(N(c4ccccc4)c4ccc5oc6ccc7sc(-c8ccc9ccccc9c8)nc7c6c5c4)cc3)cc2)cc1]

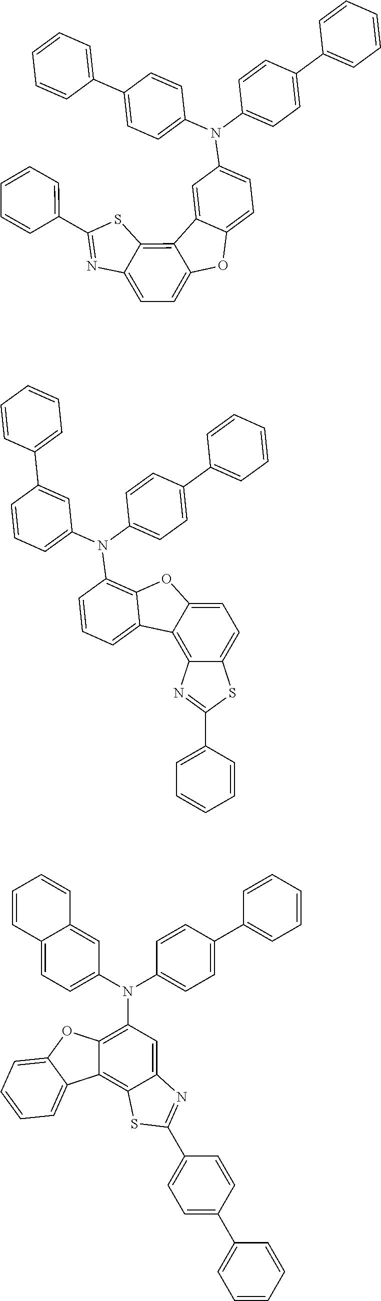 [SMILES: c1ccc(-c2ccc(N(c3ccc(-c4ccccc4)cc3)c3ccc4oc5ccc6nc(-c7ccccc7)sc6c5c4c3)cc2)cc1 ; c1ccc(-c2ccc(N(c3cccc(-c4ccccc4)c3)c3cccc4c3oc3ccc5sc(-c6ccccc6)nc5c34)cc2)cc1 ; c1ccc(-c2ccc(-c3nc4cc(N(c5ccc(-c6ccccc6)cc5)c5ccc6ccccc6c5)c5oc6ccccc6c5c4s3)cc2)cc1]

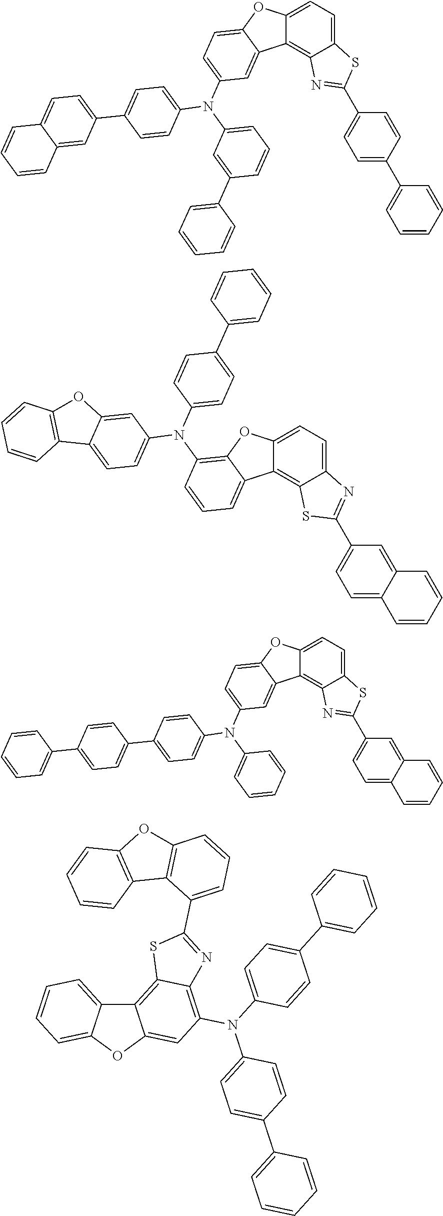

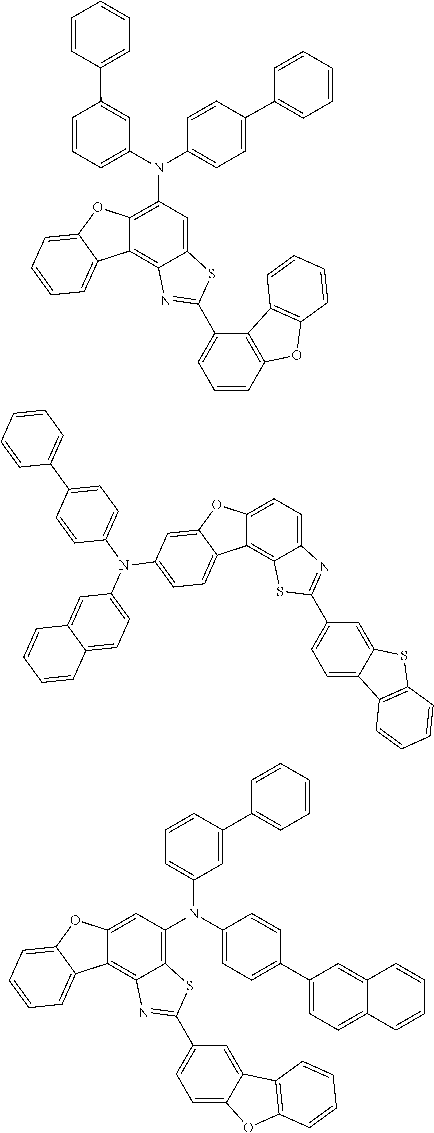 [SMILES: c1ccc(-c2ccc(N(c3cccc(-c4ccccc4)c3)c3cc4sc(-c5cccc6oc7ccccc7c56)nc4c4c3oc3ccccc34)cc2)cc1 ; c1ccc(-c2ccc(N(c3ccc4ccccc4c3)c3ccc4c(c3)oc3ccc5nc(-c6ccc7c(c6)sc6ccccc67)sc5c34)cc2)cc1 ; c1ccc(-c2cccc(N(c3ccc(-c4ccc5ccccc5c4)cc3)c3cc4oc5ccccc5c4c4nc(-c5ccc6oc7ccccc7c6c5)sc34)c2)cc1]

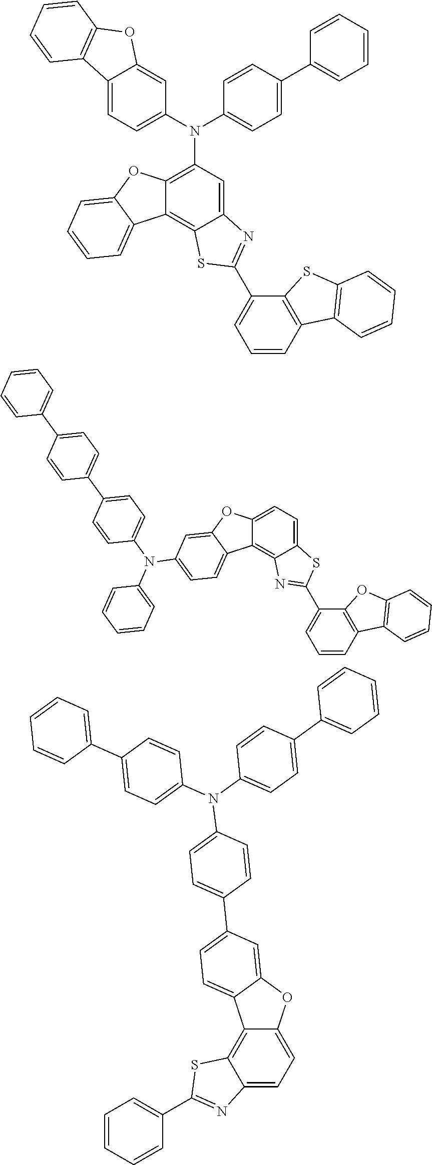

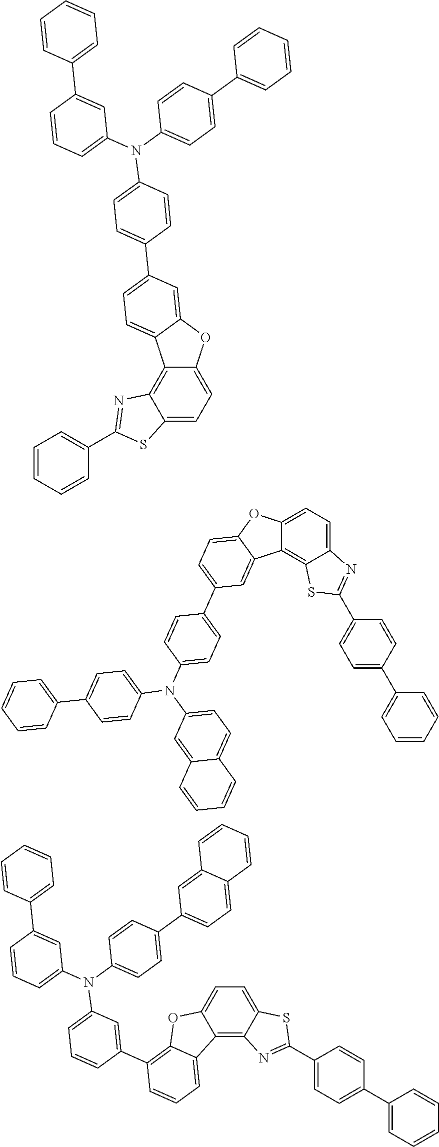

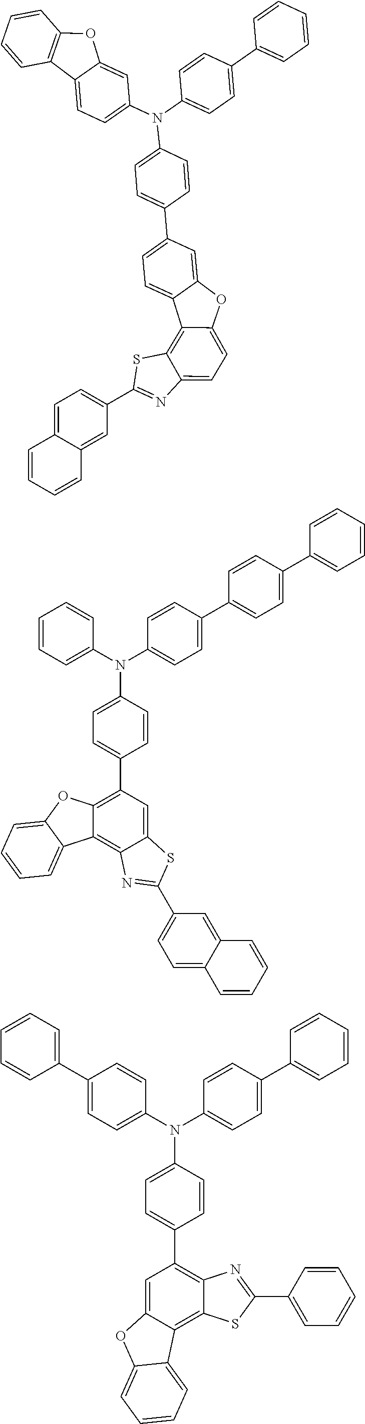 [SMILES: c1ccc(-c2ccc(N(c3ccc(-c4ccc5c(c4)oc4ccc6nc(-c7ccc8ccccc8c7)sc6c45)cc3)c3ccc4c(c3)oc3ccccc34)cc2)cc1 ; c1ccc(-c2ccc(-c3ccc(N(c4ccccc4)c4ccc(-c5cc6sc(-c7ccc8ccccc8c7)nc6c6c5oc5ccccc56)cc4)cc3)cc2)cc1 ; c1ccc(-c2ccc(N(c3ccc(-c4ccccc4)cc3)c3ccc(-c4cc5oc6ccccc6c5c5sc(-c6ccccc6)nc45)cc3)cc2)cc1]

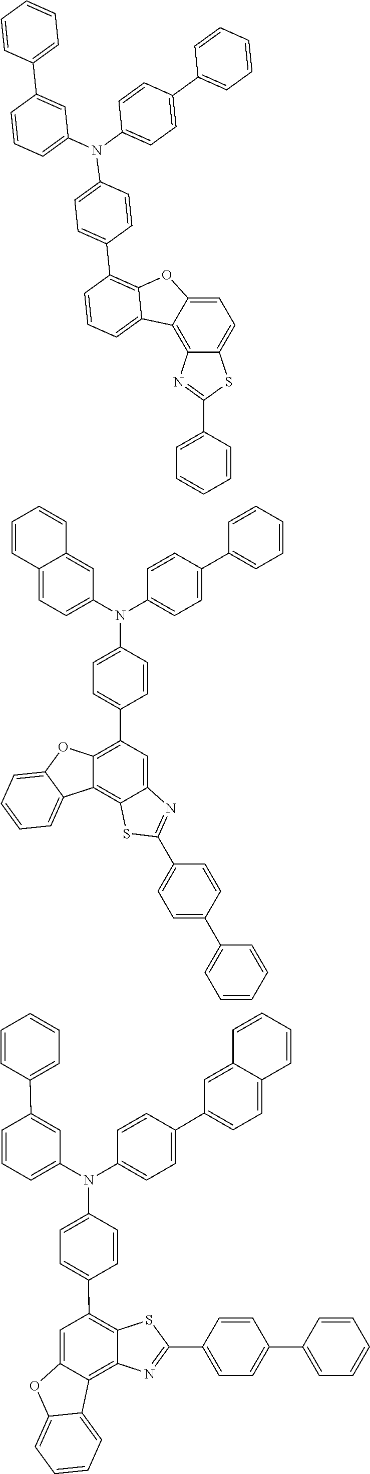

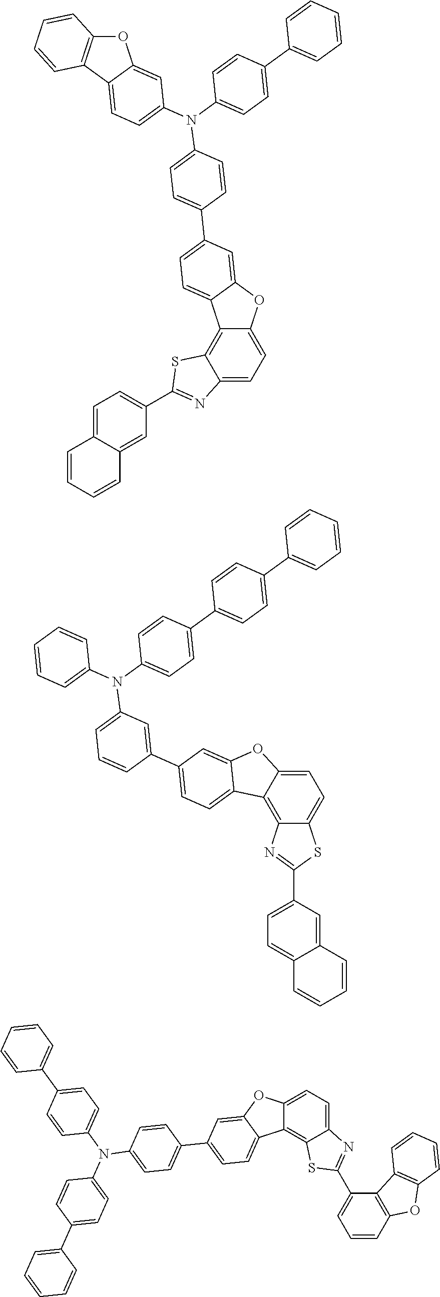 [SMILES: c1ccc(-c2ccc(N(c3ccc(-c4ccc5c(c4)oc4ccc6nc(-c7ccc8ccccc8c7)sc6c45)cc3)c3ccc4c(c3)oc3ccccc34)cc2)cc1 ; c1ccc(-c2ccc(-c3ccc(N(c4ccccc4)c4cccc(-c5ccc6c(c5)oc5ccc7sc(-c8ccc9ccccc9c8)nc7c56)c4)cc3)cc2)cc1 ; c1ccc(-c2ccc(N(c3ccc(-c4ccccc4)cc3)c3ccc(-c4ccc5c(c4)oc4ccc6nc(-c7cccc8oc9ccccc9c78)sc6c45)cc3)cc2)cc1]

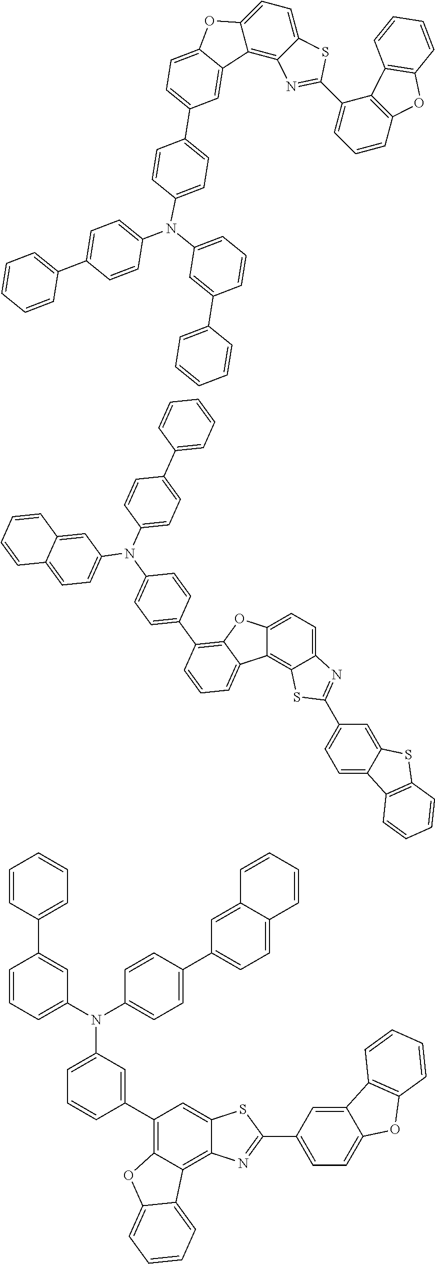

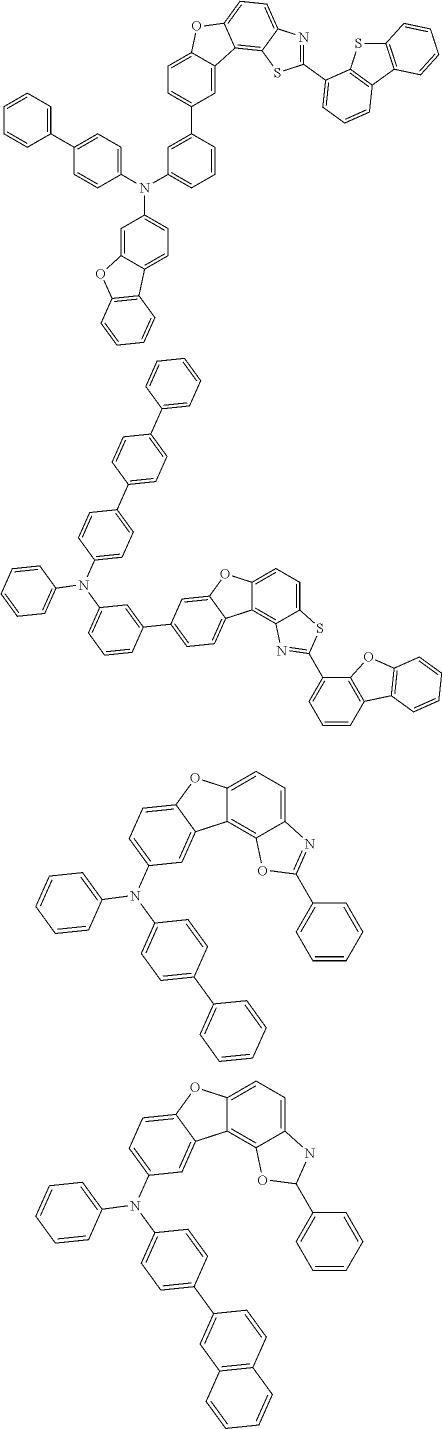

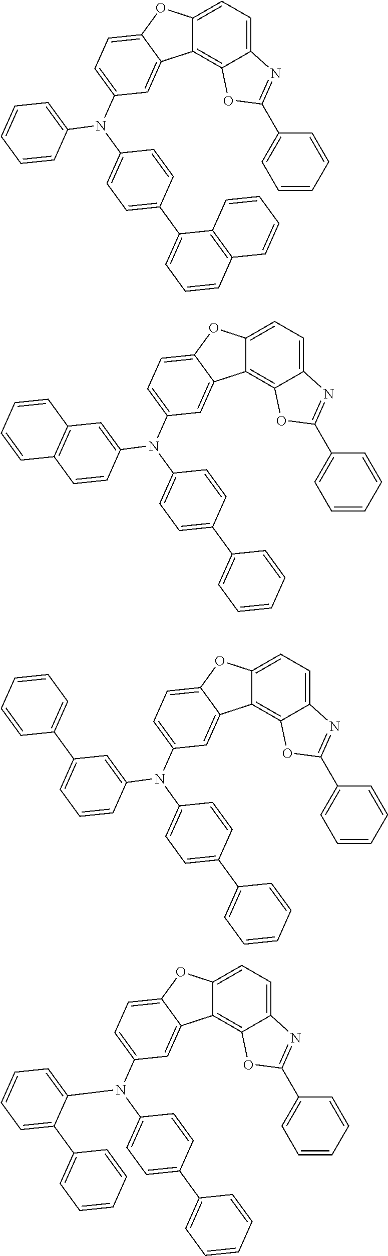

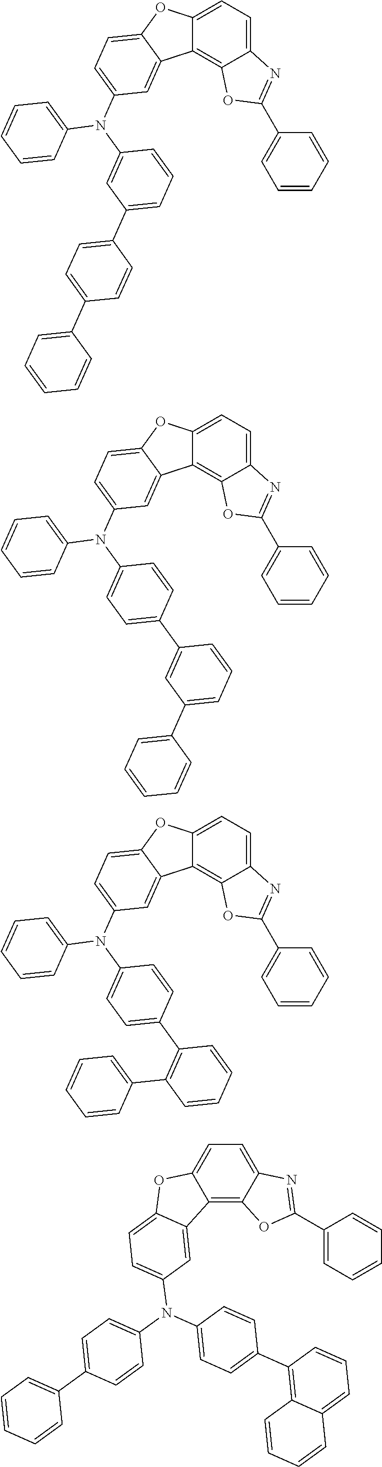

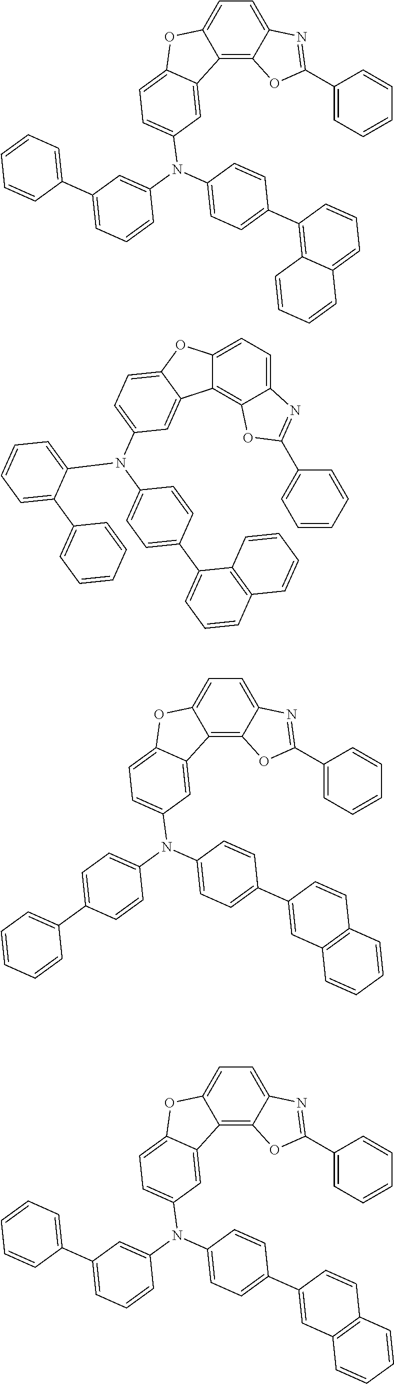

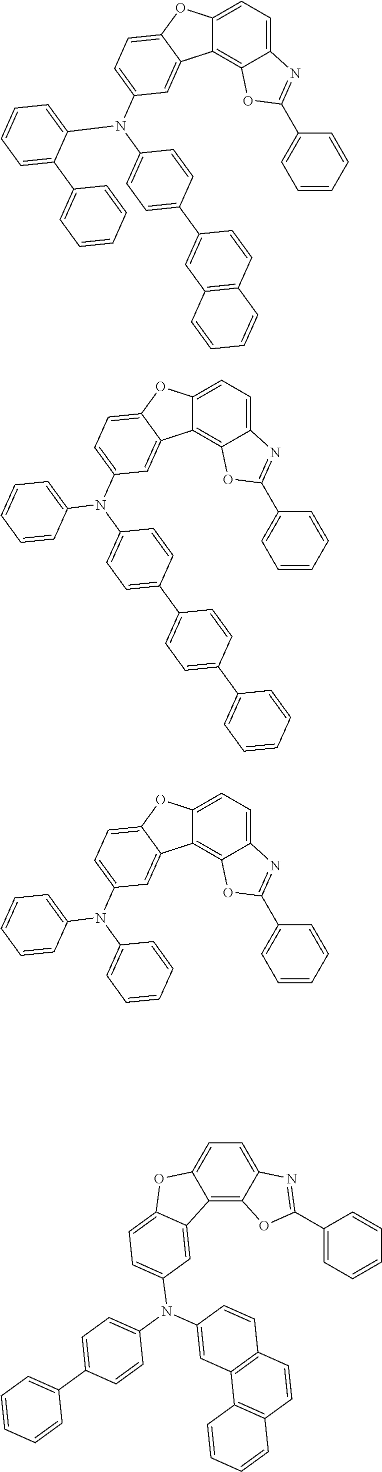

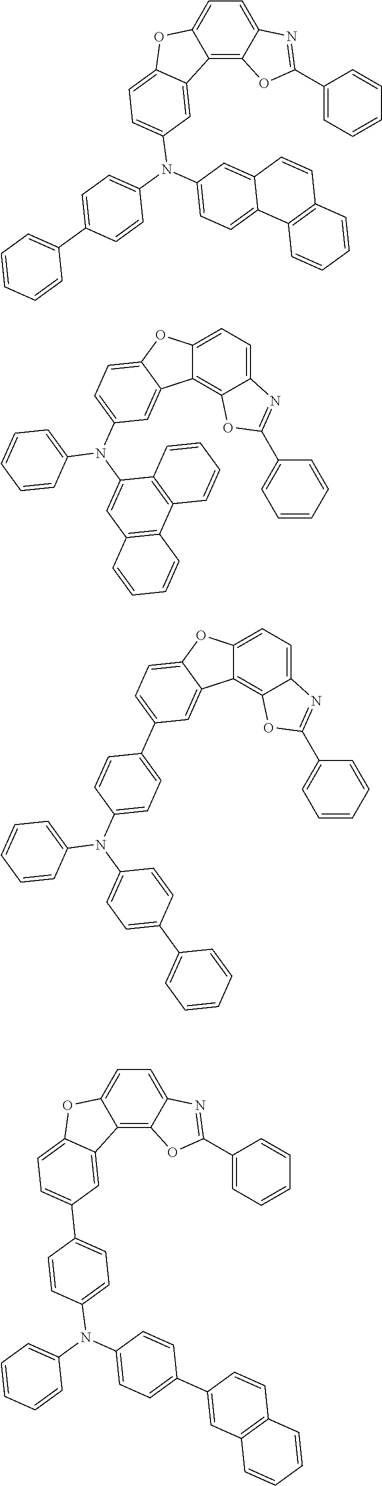 [SMILES: c1ccc(-c2ccc(N(c3ccc4c(ccc5ccccc54)c3)c3ccc4oc5ccc6nc(-c7ccccc7)oc6c5c4c3)cc2)cc1 ; c1ccc(-c2nc3ccc4oc5ccc(N(c6ccccc6)c6cc7ccccc7c7ccccc67)cc5c4c3o2)cc1 ; c1ccc(-c2ccc(N(c3ccccc3)c3ccc(-c4ccc5oc6ccc7nc(-c8ccccc8)oc7c6c5c4)cc3)cc2)cc1 ; c1ccc(-c2nc3ccc4oc5ccc(-c6ccc(N(c7ccccc7)c7ccc(-c8ccc9ccccc9c8)cc7)cc6)cc5c4c3o2)cc1]

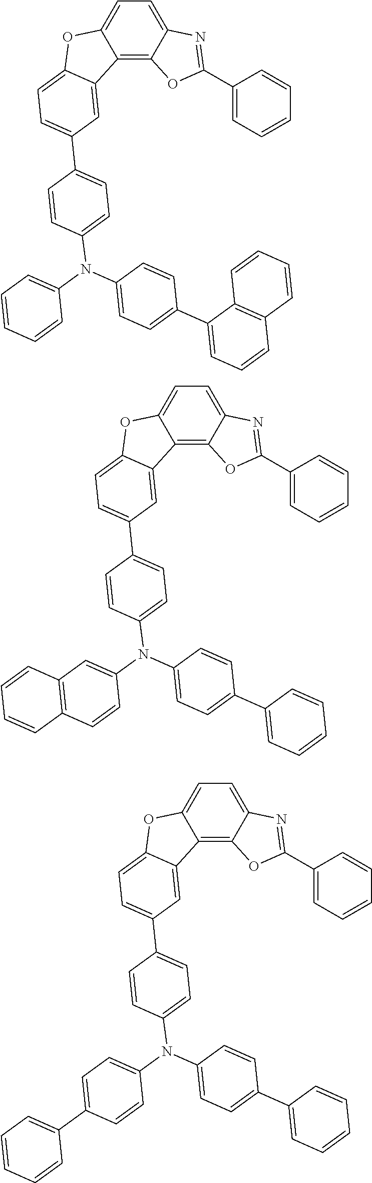

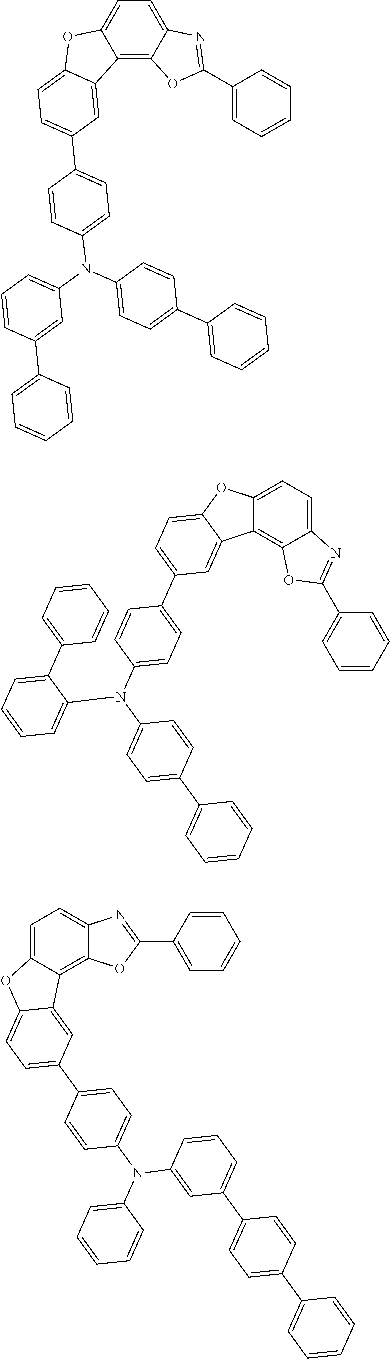 [SMILES: c1ccc(-c2ccc(N(c3ccc(-c4ccc5oc6ccc7nc(-c8ccccc8)oc7c6c5c4)cc3)c3cccc(-c4ccccc4)c3)cc2)cc1 ; c1ccc(-c2ccc(N(c3ccc(-c4ccc5oc6ccc7nc(-c8ccccc8)oc7c6c5c4)cc3)c3ccccc3-c3ccccc3)cc2)cc1 ; c1ccc(-c2ccc(-c3cccc(N(c4ccccc4)c4ccc(-c5ccc6oc7ccc8nc(-c9ccccc9)oc8c7c6c5)cc4)c3)cc2)cc1]

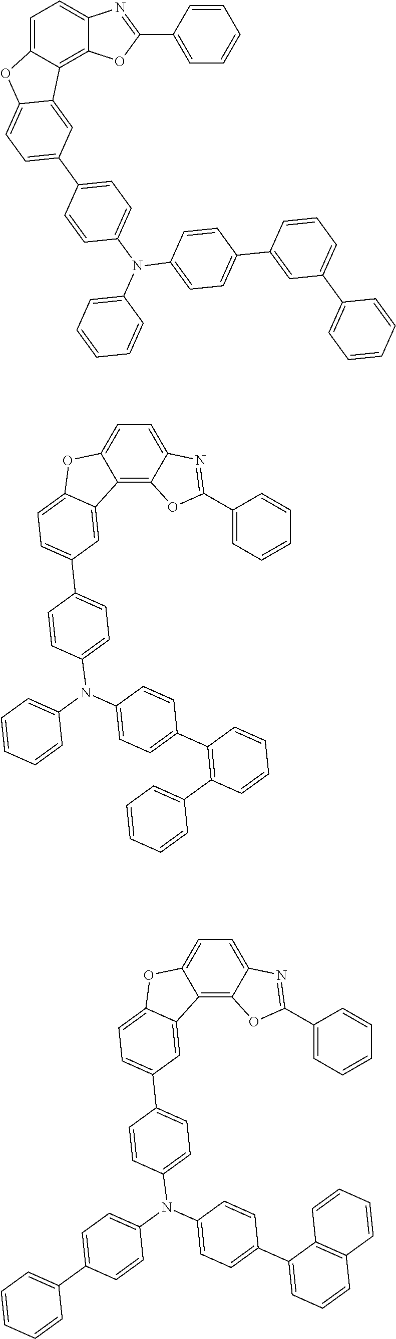 [SMILES: c1ccc(-c2cccc(-c3ccc(N(c4ccccc4)c4ccc(-c5ccc6oc7ccc8nc(-c9ccccc9)oc8c7c6c5)cc4)cc3)c2)cc1 ; c1ccc(-c2nc3ccc4oc5ccc(-c6ccc(N(c7ccccc7)c7ccc(-c8ccccc8-c8ccccc8)cc7)cc6)cc5c4c3o2)cc1 ; c1ccc(-c2ccc(N(c3ccc(-c4ccc5oc6ccc7nc(-c8ccccc8)oc7c6c5c4)cc3)c3ccc(-c4cccc5ccccc45)cc3)cc2)cc1]

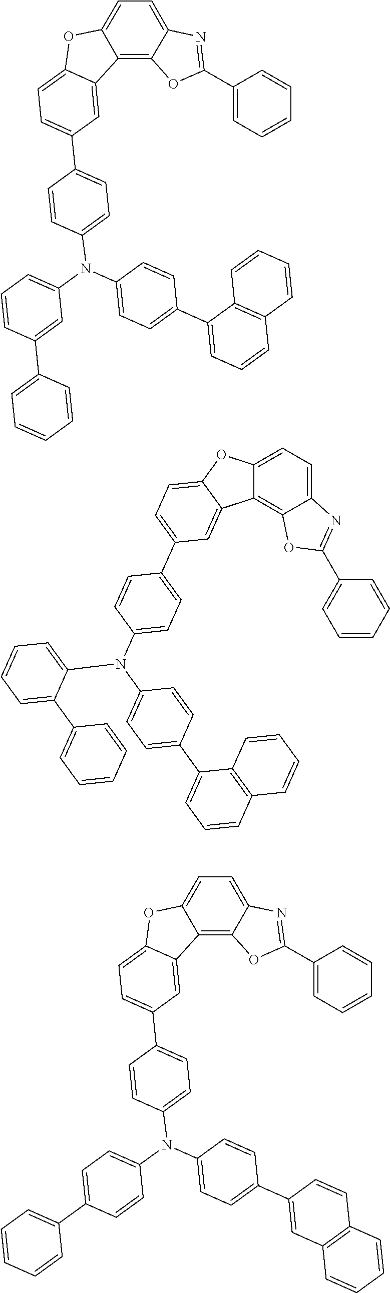 [SMILES: c1ccc(-c2cccc(N(c3ccc(-c4ccc5oc6ccc7nc(-c8ccccc8)oc7c6c5c4)cc3)c3ccc(-c4cccc5ccccc45)cc3)c2)cc1 ; c1ccc(-c2nc3ccc4oc5ccc(-c6ccc(N(c7ccc(-c8cccc9ccccc89)cc7)c7ccccc7-c7ccccc7)cc6)cc5c4c3o2)cc1 ; c1ccc(-c2ccc(N(c3ccc(-c4ccc5ccccc5c4)cc3)c3ccc(-c4ccc5oc6ccc7nc(-c8ccccc8)oc7c6c5c4)cc3)cc2)cc1]

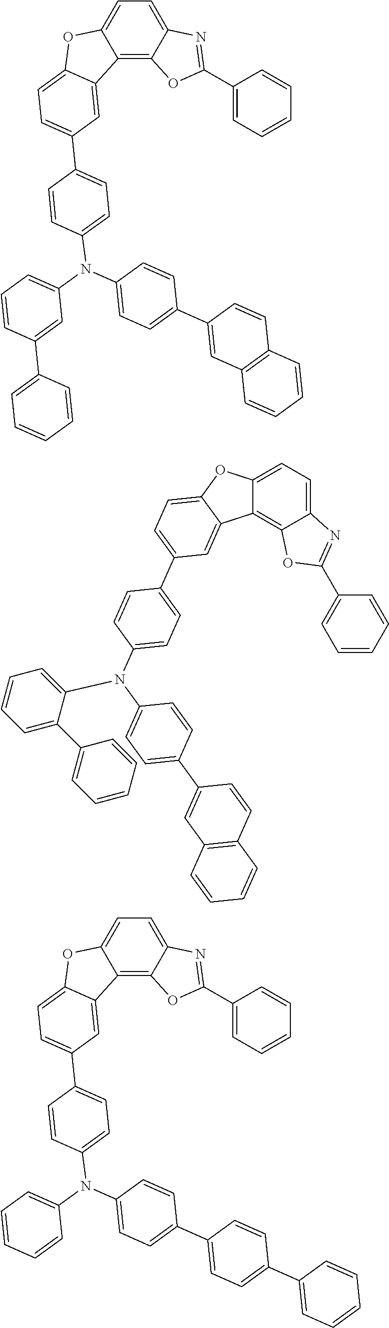 [SMILES: c1ccc(-c2cccc(N(c3ccc(-c4ccc5ccccc5c4)cc3)c3ccc(-c4ccc5oc6ccc7nc(-c8ccccc8)oc7c6c5c4)cc3)c2)cc1 ; c1ccc(-c2nc3ccc4oc5ccc(-c6ccc(N(c7ccc(-c8ccc9ccccc9c8)cc7)c7ccccc7-c7ccccc7)cc6)cc5c4c3o2)cc1 ; c1ccc(-c2ccc(-c3ccc(N(c4ccccc4)c4ccc(-c5ccc6oc7ccc8nc(-c9ccccc9)oc8c7c6c5)cc4)cc3)cc2)cc1]

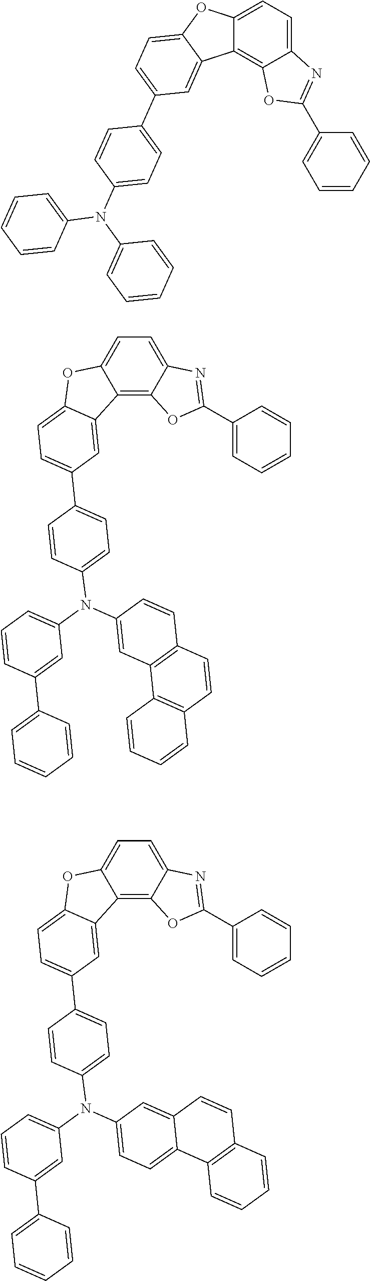

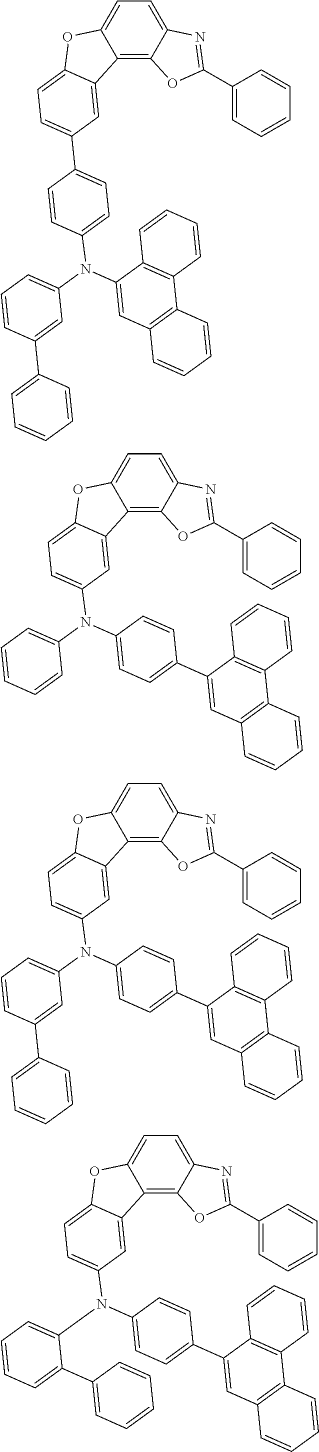 [SMILES: c1ccc(-c2cccc(N(c3ccc(-c4ccc5oc6ccc7nc(-c8ccccc8)oc7c6c5c4)cc3)c3cc4ccccc4c4ccccc34)c2)cc1 ; c1ccc(-c2nc3ccc4oc5ccc(N(c6ccccc6)c6ccc(-c7cc8ccccc8c8ccccc78)cc6)cc5c4c3o2)cc1 ; c1ccc(-c2cccc(N(c3ccc(-c4cc5ccccc5c5ccccc45)cc3)c3ccc4oc5ccc6nc(-c7ccccc7)oc6c5c4c3)c2)cc1 ; c1ccc(-c2nc3ccc4oc5ccc(N(c6ccc(-c7cc8ccccc8c8ccccc78)cc6)c6ccccc6-c6ccccc6)cc5c4c3o2)cc1]

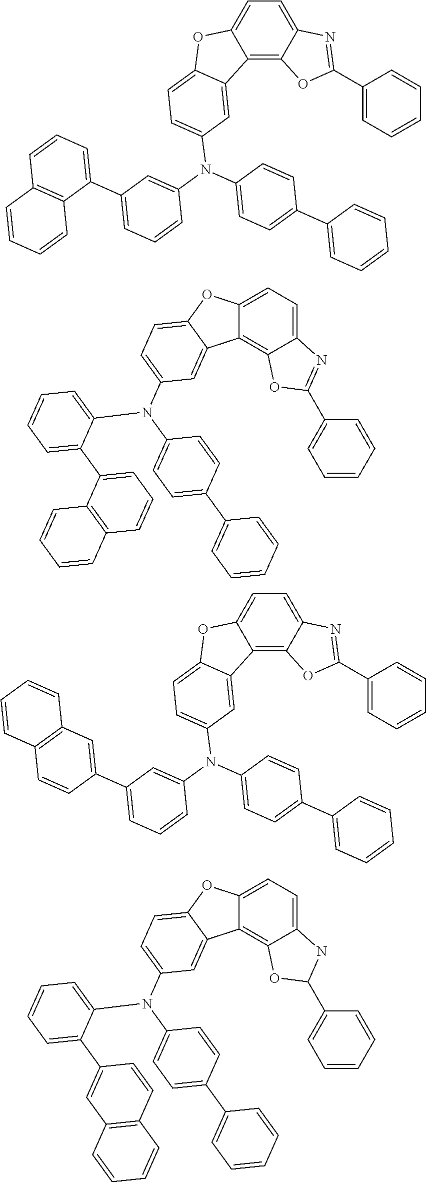

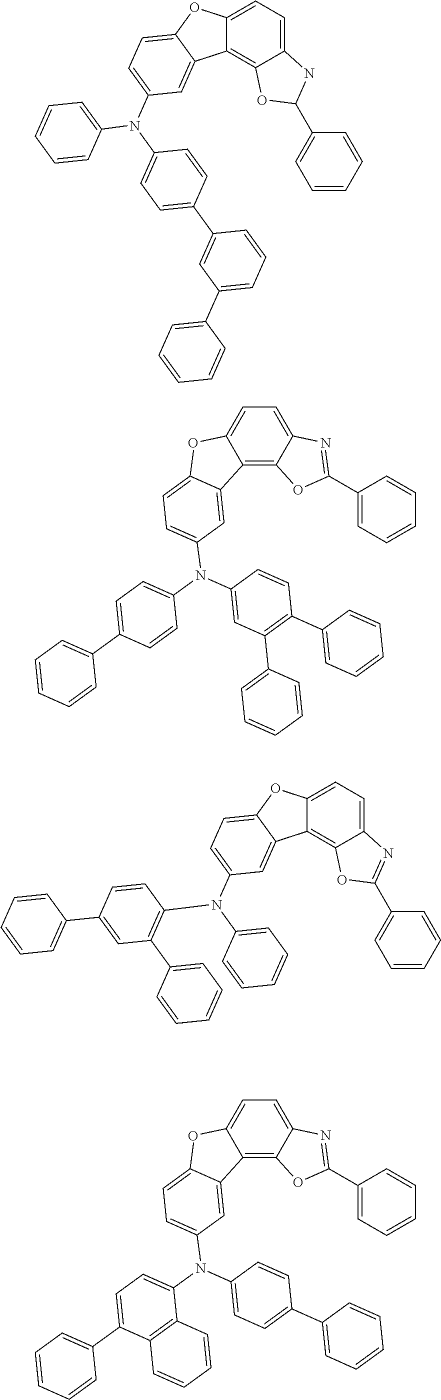

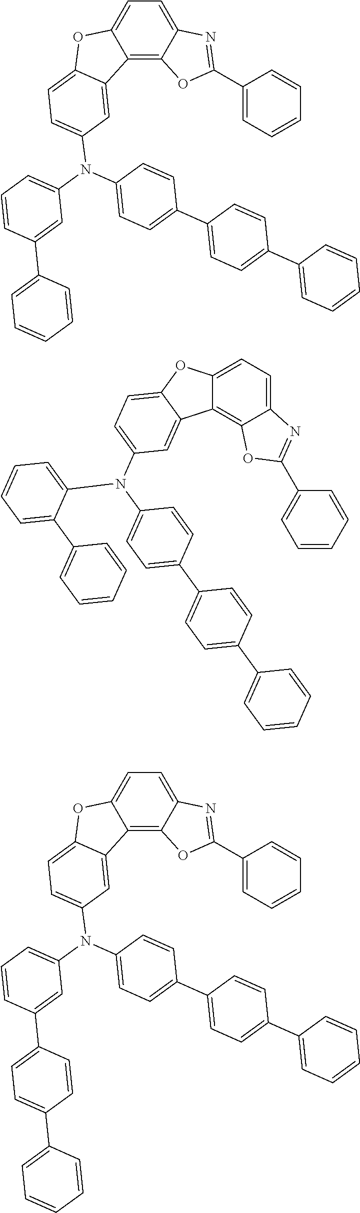 [SMILES: c1ccc(-c2ccc(-c3ccc(N(c4cccc(-c5ccccc5)c4)c4ccc5oc6ccc7nc(-c8ccccc8)oc7c6c5c4)cc3)cc2)cc1 ; c1ccc(-c2ccc(-c3ccc(N(c4ccc5oc6ccc7nc(-c8ccccc8)oc7c6c5c4)c4ccccc4-c4ccccc4)cc3)cc2)cc1 ; c1ccc(-c2ccc(-c3ccc(N(c4cccc(-c5ccc(-c6ccccc6)cc5)c4)c4ccc5oc6ccc7nc(-c8ccccc8)oc7c6c5c4)cc3)cc2)cc1]

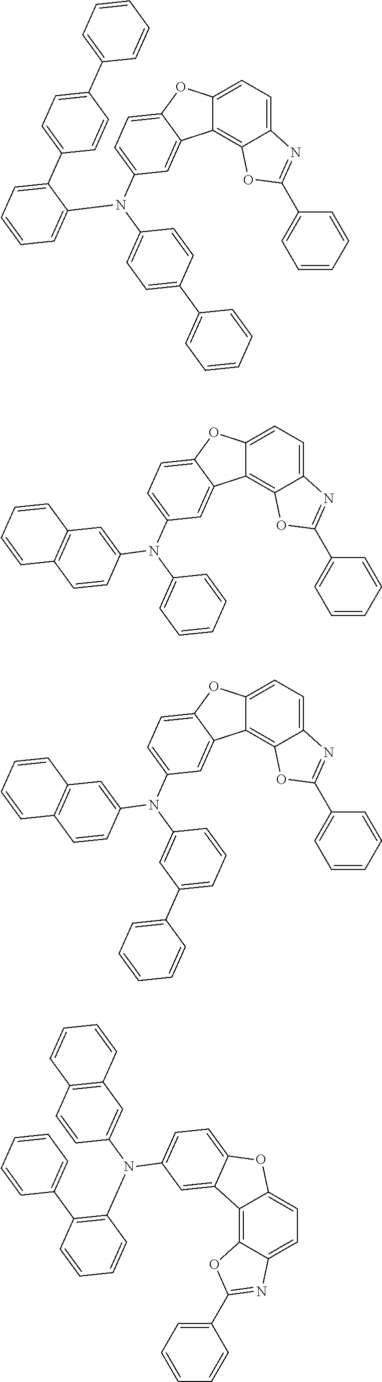 [SMILES: c1ccc(-c2ccc(-c3ccccc3N(c3ccc(-c4ccccc4)cc3)c3ccc4oc5ccc6nc(-c7ccccc7)oc6c5c4c3)cc2)cc1 ; c1ccc(-c2nc3ccc4oc5ccc(N(c6ccccc6)c6ccc7ccccc7c6)cc5c4c3o2)cc1 ; c1ccc(-c2cccc(N(c3ccc4ccccc4c3)c3ccc4oc5ccc6nc(-c7ccccc7)oc6c5c4c3)c2)cc1 ; c1ccc(-c2nc3ccc4oc5ccc(N(c6ccc7ccccc7c6)c6ccccc6-c6ccccc6)cc5c4c3o2)cc1]

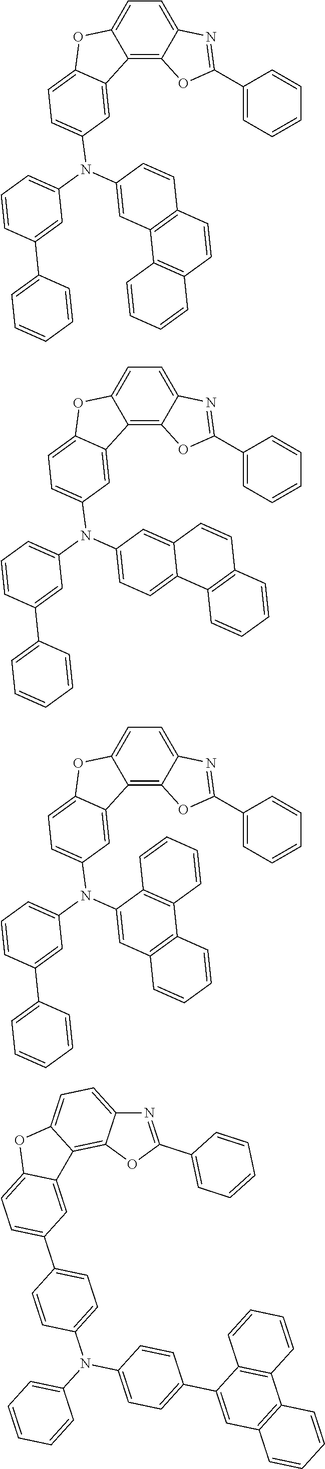

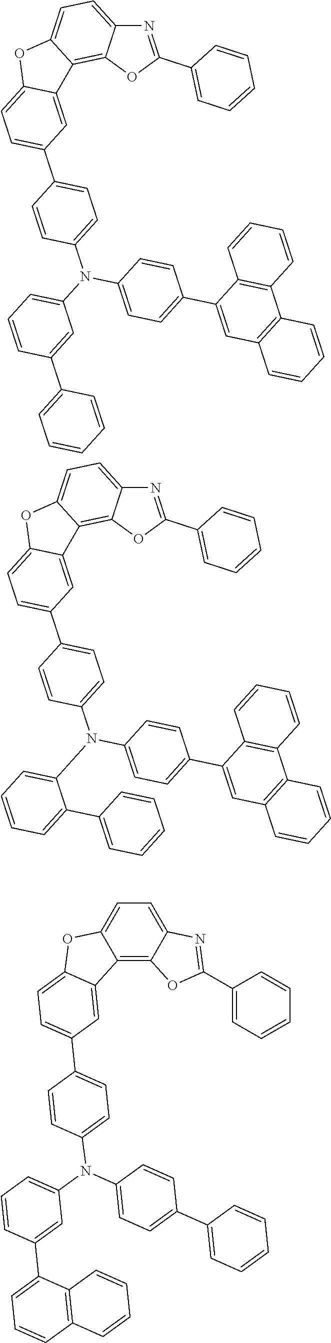

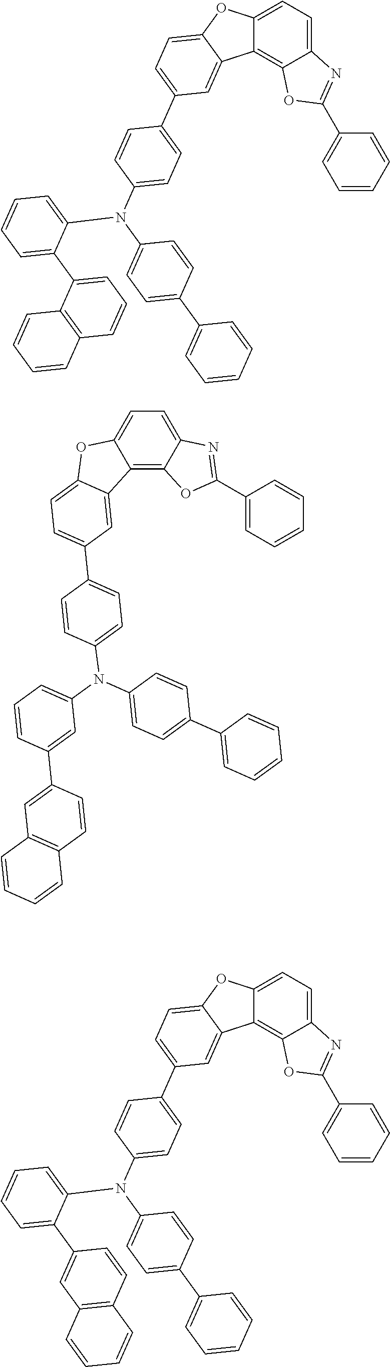 [SMILES: c1ccc(-c2ccc(N(c3ccc(-c4ccc5oc6ccc7nc(-c8ccccc8)oc7c6c5c4)cc3)c3ccccc3-c3cccc4ccccc34)cc2)cc1 ; c1ccc(-c2ccc(N(c3ccc(-c4ccc5oc6ccc7nc(-c8ccccc8)oc7c6c5c4)cc3)c3cccc(-c4ccc5ccccc5c4)c3)cc2)cc1 ; c1ccc(-c2ccc(N(c3ccc(-c4ccc5oc6ccc7nc(-c8ccccc8)oc7c6c5c4)cc3)c3ccccc3-c3ccc4ccccc4c3)cc2)cc1]

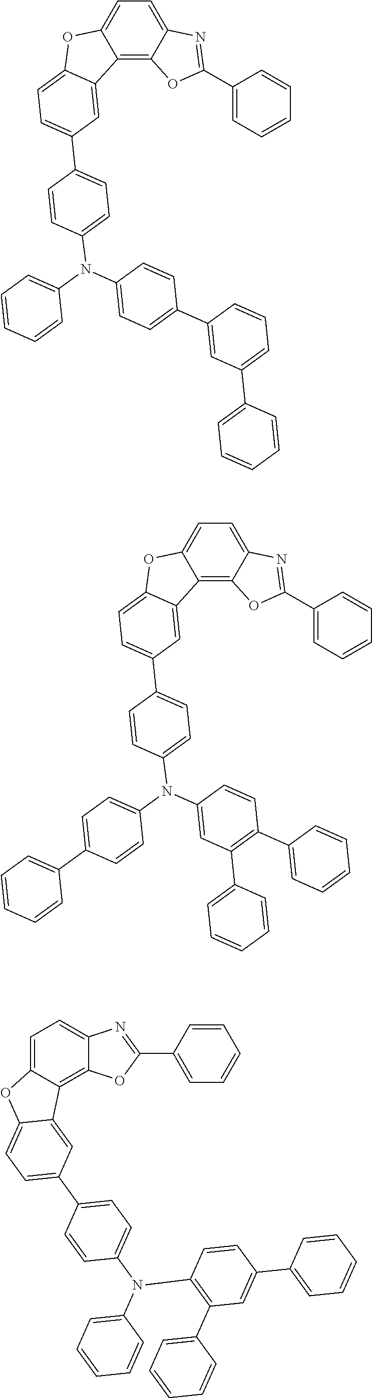 [SMILES: c1ccc(-c2cccc(-c3ccc(N(c4ccccc4)c4ccc(-c5ccc6oc7ccc8nc(-c9ccccc9)oc8c7c6c5)cc4)cc3)c2)cc1 ; c1ccc(-c2ccc(N(c3ccc(-c4ccc5oc6ccc7nc(-c8ccccc8)oc7c6c5c4)cc3)c3ccc(-c4ccccc4)c(-c4ccccc4)c3)cc2)cc1 ; c1ccc(-c2ccc(N(c3ccccc3)c3ccc(-c4ccc5oc6ccc7nc(-c8ccccc8)oc7c6c5c4)cc3)c(-c3ccccc3)c2)cc1]

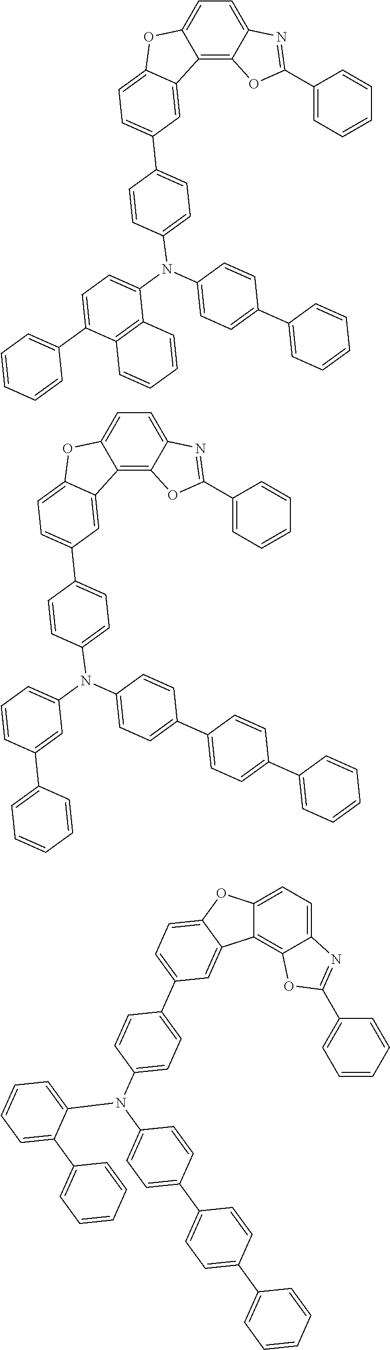

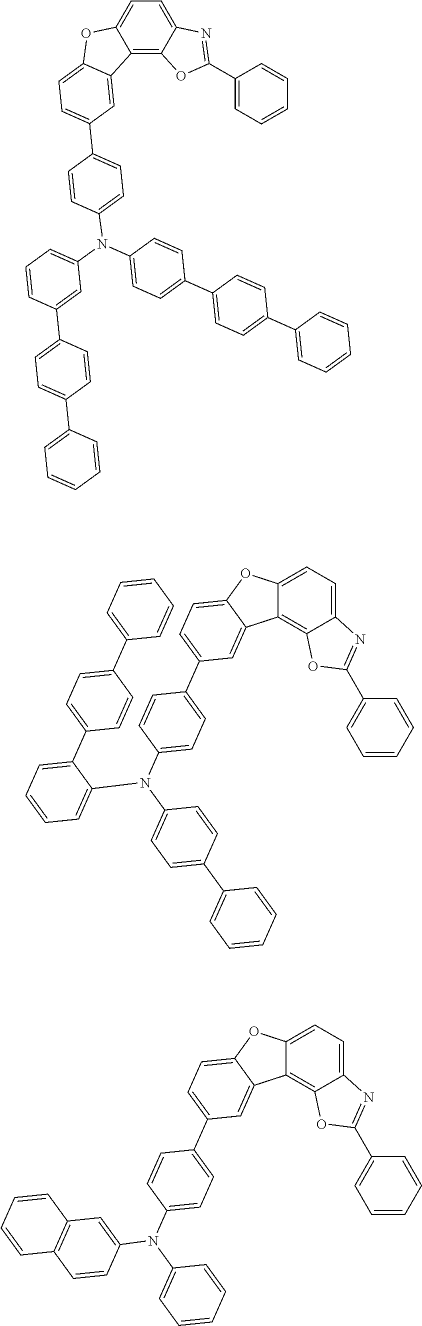 [SMILES: c1ccc(-c2ccc(-c3ccc(N(c4ccc(-c5ccc6oc7ccc8nc(-c9ccccc9)oc8c7c6c5)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4ccc5oc6ccc7nc(-c8ccccc8)oc7c6c5c4)cc3)cc2)cc1 ; c1ccc(-c2nc3ccc4oc5ccc(-c6ccc(N(c7ccccc7)c7ccc8ccccc8c7)cc6)cc5c4c3o2)cc1]

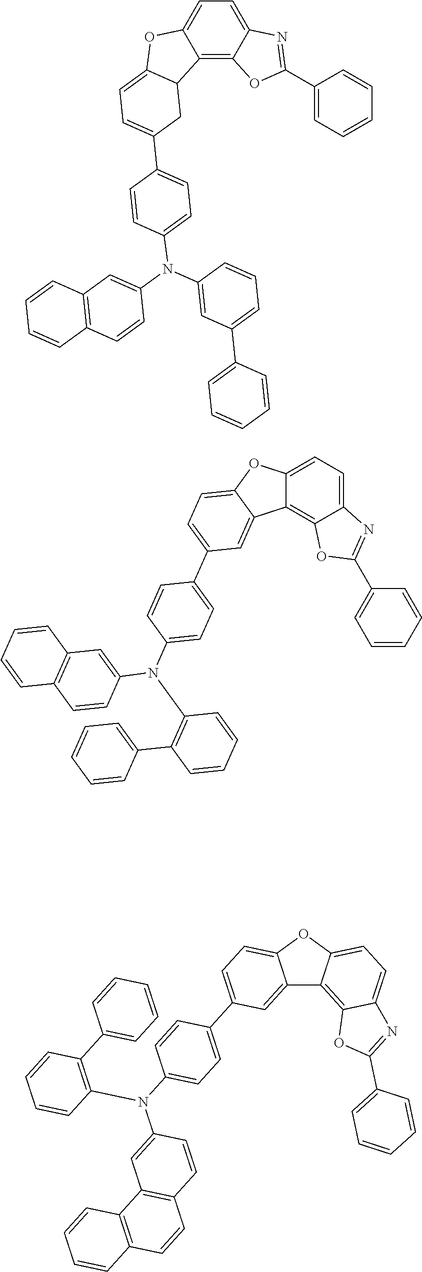

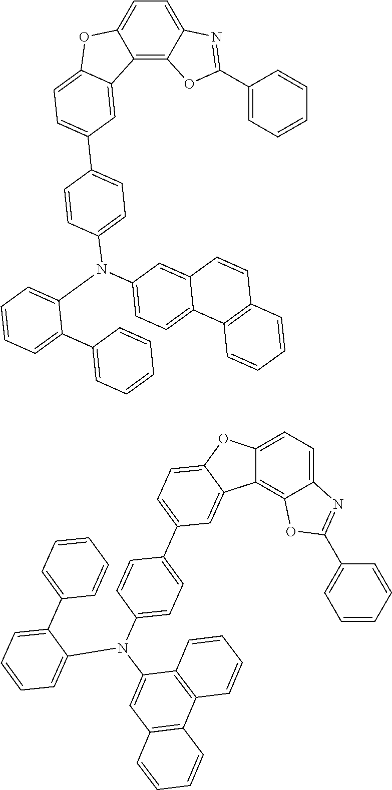 [SMILES: c1ccc(-c2nc3ccc4oc5ccc(-c6ccc(N(c7ccc8c(ccc9ccccc98)c7)c7ccccc7-c7ccccc7)cc6)cc5c4c3o2)cc1 ; c1ccc(-c2nc3ccc4oc5ccc(-c6ccc(N(c7ccccc7-c7ccccc7)c7cc8ccccc8c8ccccc78)cc6)cc5c4c3o2)cc1]

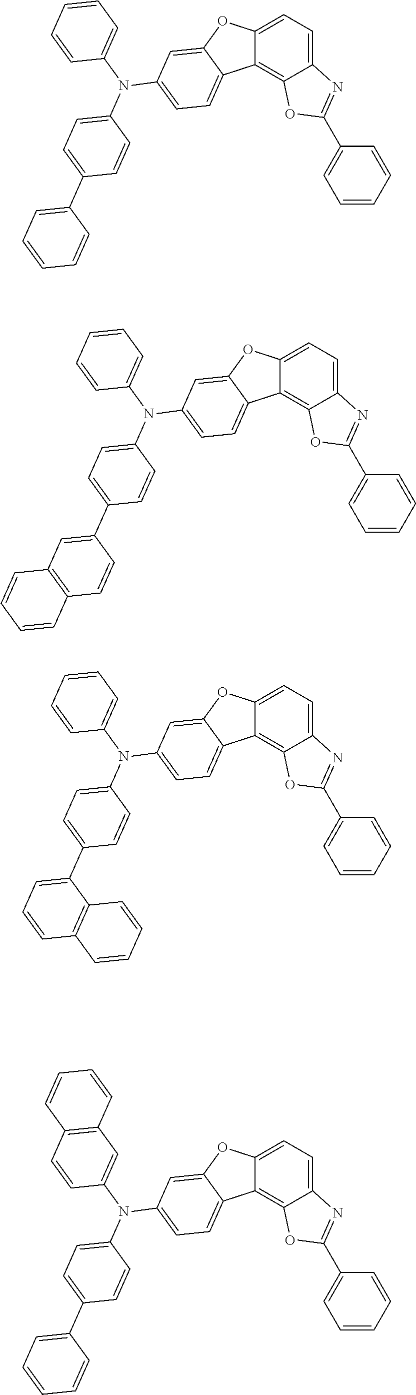 [SMILES: c1ccc(-c2ccc(N(c3ccccc3)c3ccc4c(c3)oc3ccc5nc(-c6ccccc6)oc5c34)cc2)cc1 ; c1ccc(-c2nc3ccc4oc5cc(N(c6ccccc6)c6ccc(-c7ccc8ccccc8c7)cc6)ccc5c4c3o2)cc1 ; c1ccc(-c2nc3ccc4oc5cc(N(c6ccccc6)c6ccc(-c7cccc8ccccc78)cc6)ccc5c4c3o2)cc1 ; c1ccc(-c2ccc(N(c3ccc4ccccc4c3)c3ccc4c(c3)oc3ccc5nc(-c6ccccc6)oc5c34)cc2)cc1]

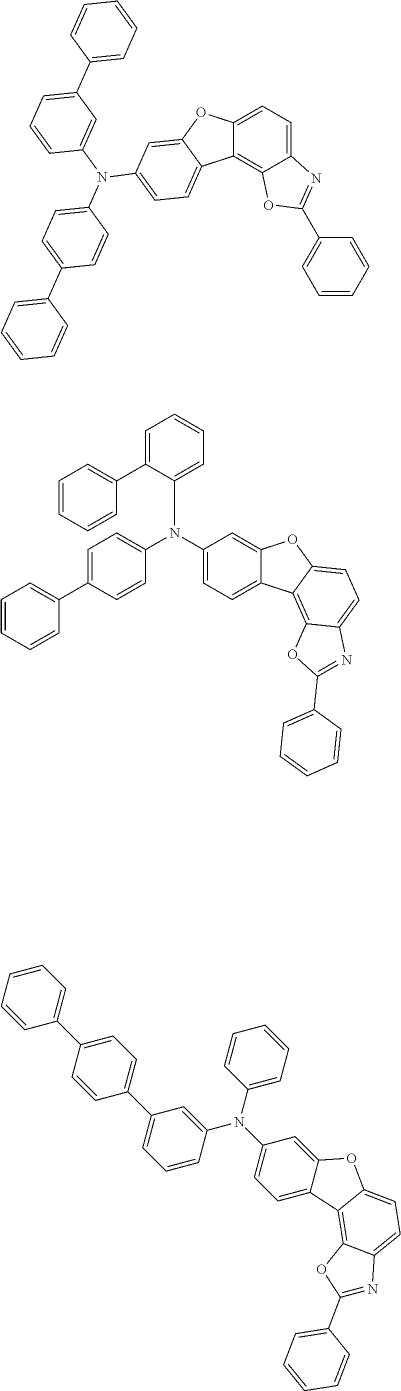 [SMILES: c1ccc(-c2ccc(N(c3cccc(-c4ccccc4)c3)c3ccc4c(c3)oc3ccc5nc(-c6ccccc6)oc5c34)cc2)cc1 ; c1ccc(-c2ccc(N(c3ccc4c(c3)oc3ccc5nc(-c6ccccc6)oc5c34)c3ccccc3-c3ccccc3)cc2)cc1 ; c1ccc(-c2ccc(-c3cccc(N(c4ccccc4)c4ccc5c(c4)oc4ccc6nc(-c7ccccc7)oc6c45)c3)cc2)cc1]

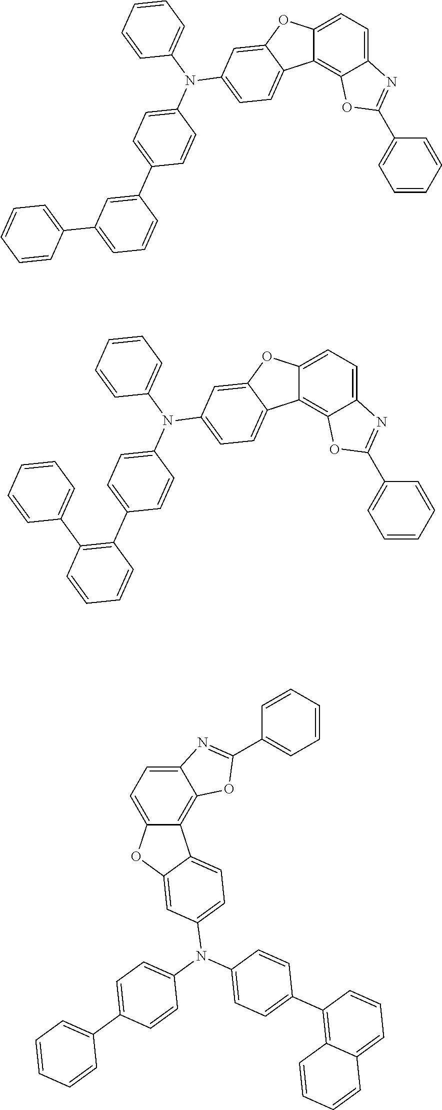 [SMILES: c1ccc(-c2cccc(-c3ccc(N(c4ccccc4)c4ccc5c(c4)oc4ccc6nc(-c7ccccc7)oc6c45)cc3)c2)cc1 ; c1ccc(-c2nc3ccc4oc5cc(N(c6ccccc6)c6ccc(-c7ccccc7-c7ccccc7)cc6)ccc5c4c3o2)cc1 ; c1ccc(-c2ccc(N(c3ccc(-c4cccc5ccccc45)cc3)c3ccc4c(c3)oc3ccc5nc(-c6ccccc6)oc5c34)cc2)cc1]

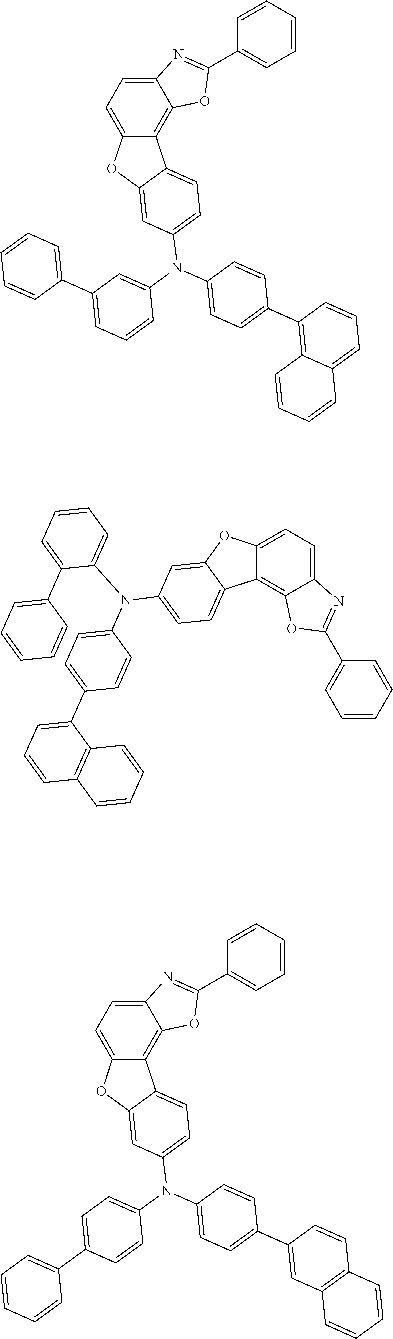 [SMILES: c1ccc(-c2cccc(N(c3ccc(-c4cccc5ccccc45)cc3)c3ccc4c(c3)oc3ccc5nc(-c6ccccc6)oc5c34)c2)cc1 ; c1ccc(-c2nc3ccc4oc5cc(N(c6ccc(-c7cccc8ccccc78)cc6)c6ccccc6-c6ccccc6)ccc5c4c3o2)cc1 ; c1ccc(-c2ccc(N(c3ccc(-c4ccc5ccccc5c4)cc3)c3ccc4c(c3)oc3ccc5nc(-c6ccccc6)oc5c34)cc2)cc1]

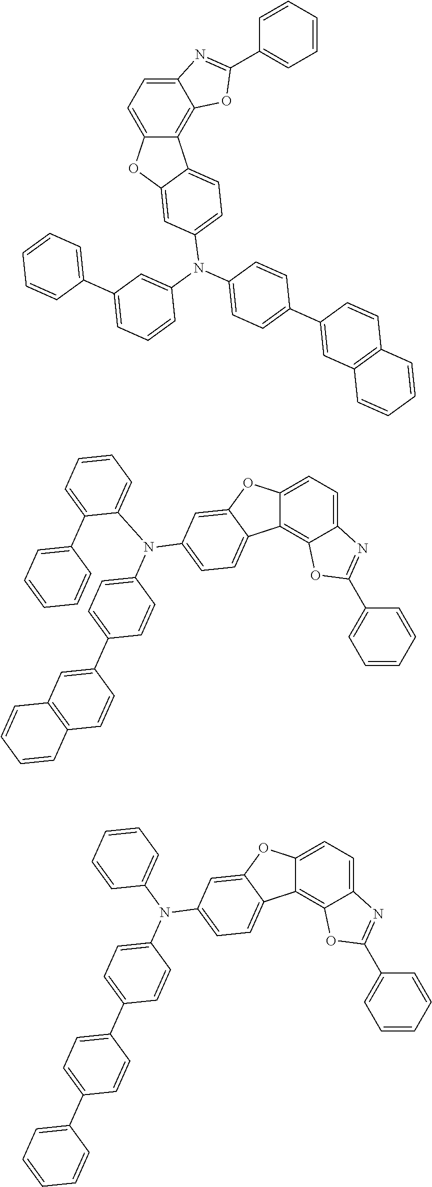 [SMILES: c1ccc(-c2cccc(N(c3ccc(-c4ccc5ccccc5c4)cc3)c3ccc4c(c3)oc3ccc5nc(-c6ccccc6)oc5c34)c2)cc1 ; c1ccc(-c2nc3ccc4oc5cc(N(c6ccc(-c7ccc8ccccc8c7)cc6)c6ccccc6-c6ccccc6)ccc5c4c3o2)cc1 ; c1ccc(-c2ccc(-c3ccc(N(c4ccccc4)c4ccc5c(c4)oc4ccc6nc(-c7ccccc7)oc6c45)cc3)cc2)cc1]

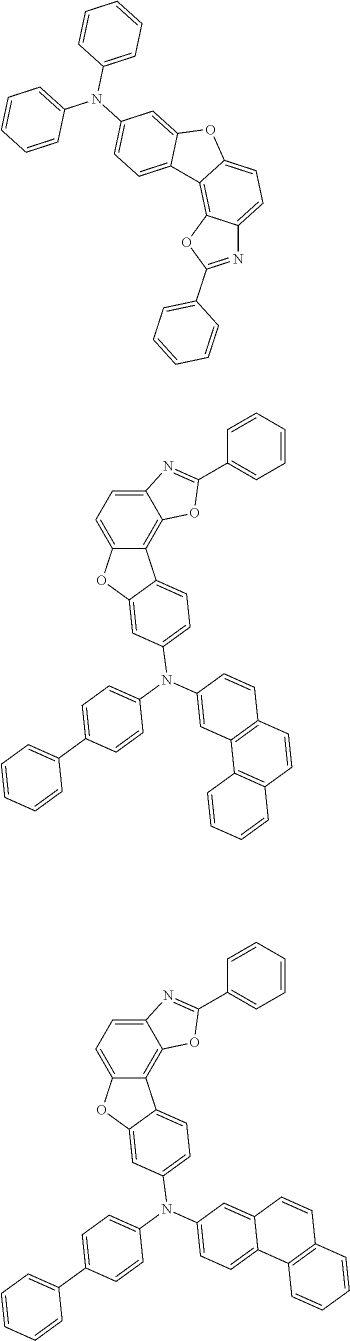 [SMILES: c1ccc(-c2nc3ccc4oc5cc(N(c6ccccc6)c6ccccc6)ccc5c4c3o2)cc1 ; c1ccc(-c2ccc(N(c3ccc4c(c3)oc3ccc5nc(-c6ccccc6)oc5c34)c3ccc4ccc5ccccc5c4c3)cc2)cc1 ; c1ccc(-c2ccc(N(c3ccc4c(ccc5ccccc54)c3)c3ccc4c(c3)oc3ccc5nc(-c6ccccc6)oc5c34)cc2)cc1]

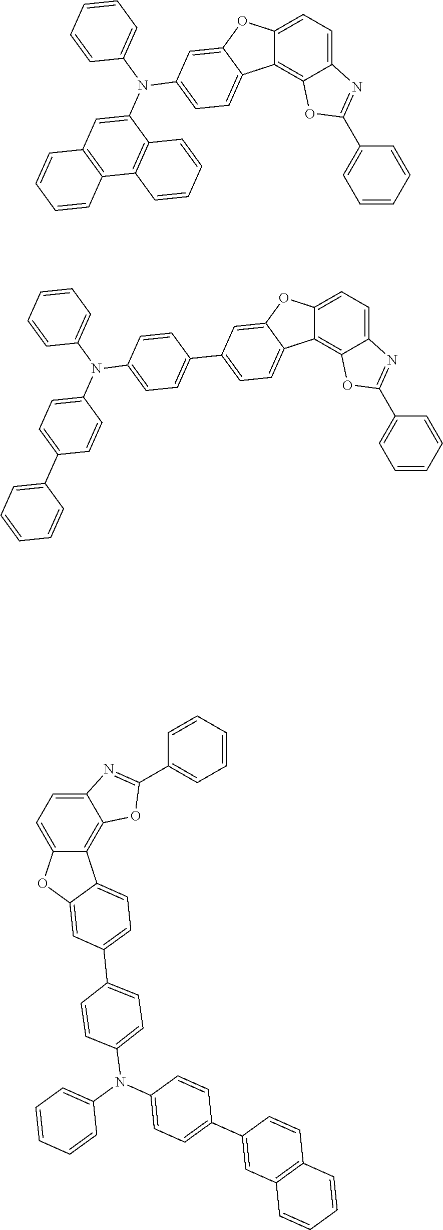 [SMILES: c1ccc(-c2nc3ccc4oc5cc(N(c6ccccc6)c6cc7ccccc7c7ccccc67)ccc5c4c3o2)cc1 ; c1ccc(-c2ccc(N(c3ccccc3)c3ccc(-c4ccc5c(c4)oc4ccc6nc(-c7ccccc7)oc6c45)cc3)cc2)cc1 ; c1ccc(-c2nc3ccc4oc5cc(-c6ccc(N(c7ccccc7)c7ccc(-c8ccc9ccccc9c8)cc7)cc6)ccc5c4c3o2)cc1]

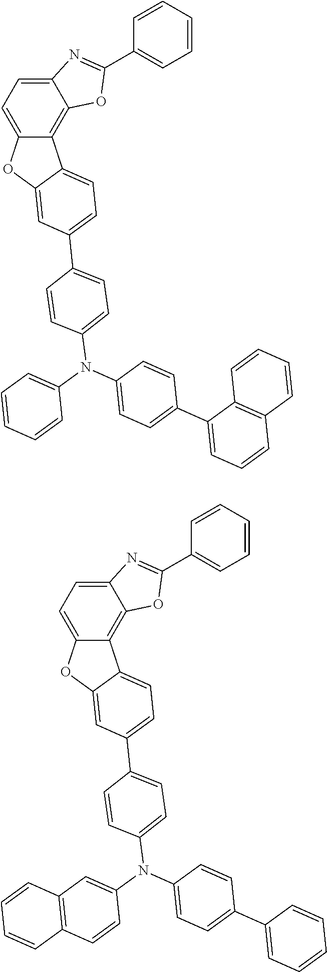

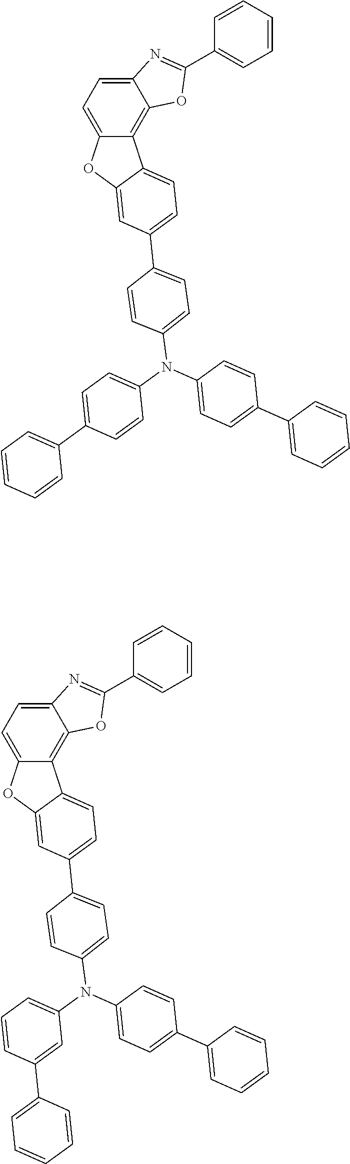 [SMILES: c1ccc(-c2ccc(N(c3ccc(-c4ccccc4)cc3)c3ccc(-c4ccc5c(c4)oc4ccc6nc(-c7ccccc7)oc6c45)cc3)cc2)cc1 ; c1ccc(-c2ccc(N(c3ccc(-c4ccc5c(c4)oc4ccc6nc(-c7ccccc7)oc6c45)cc3)c3cccc(-c4ccccc4)c3)cc2)cc1]

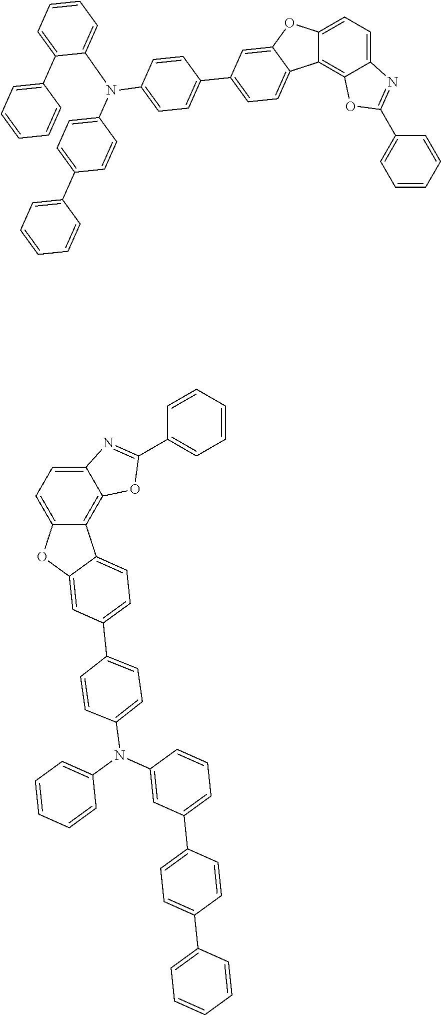 [SMILES: c1ccc(-c2ccc(N(c3ccc(-c4ccc5c(c4)oc4ccc6nc(-c7ccccc7)oc6c45)cc3)c3ccccc3-c3ccccc3)cc2)cc1 ; c1ccc(-c2ccc(-c3cccc(N(c4ccccc4)c4ccc(-c5ccc6c(c5)oc5ccc7nc(-c8ccccc8)oc7c56)cc4)c3)cc2)cc1]

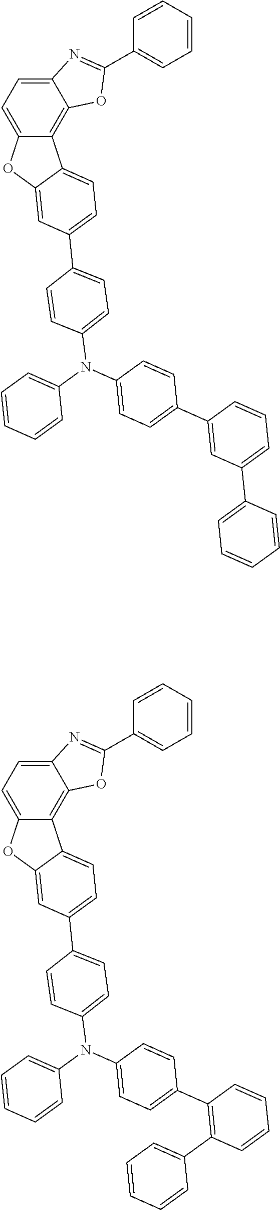 [SMILES: c1ccc(-c2cccc(-c3ccc(N(c4ccccc4)c4ccc(-c5ccc6c(c5)oc5ccc7nc(-c8ccccc8)oc7c56)cc4)cc3)c2)cc1 ; c1ccc(-c2nc3ccc4oc5cc(-c6ccc(N(c7ccccc7)c7ccc(-c8ccccc8-c8ccccc8)cc7)cc6)ccc5c4c3o2)cc1]

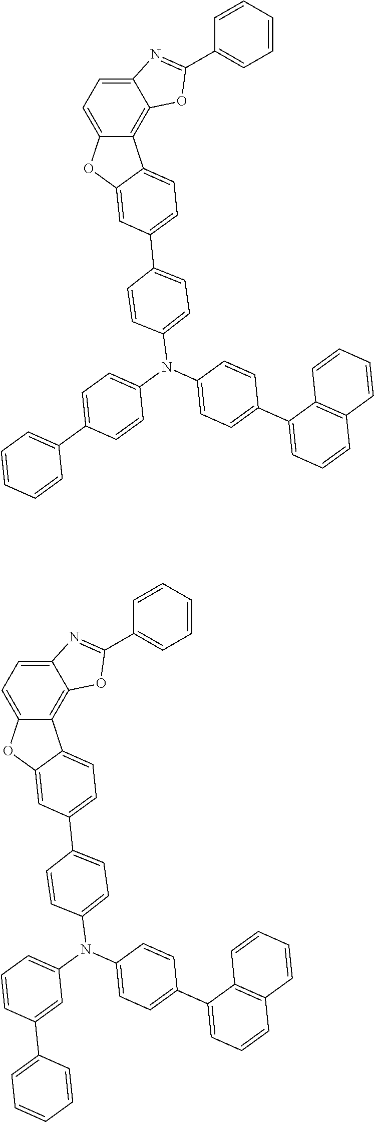 [SMILES: c1ccc(-c2ccc(N(c3ccc(-c4ccc5c(c4)oc4ccc6nc(-c7ccccc7)oc6c45)cc3)c3ccc(-c4cccc5ccccc45)cc3)cc2)cc1 ; c1ccc(-c2cccc(N(c3ccc(-c4ccc5c(c4)oc4ccc6nc(-c7ccccc7)oc6c45)cc3)c3ccc(-c4cccc5ccccc45)cc3)c2)cc1]

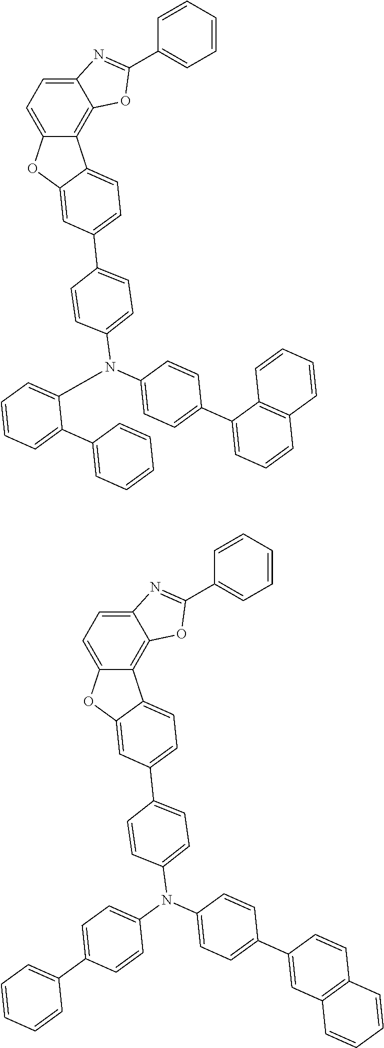 [SMILES: c1ccc(-c2nc3ccc4oc5cc(-c6ccc(N(c7ccc(-c8cccc9ccccc89)cc7)c7ccccc7-c7ccccc7)cc6)ccc5c4c3o2)cc1 ; c1ccc(-c2ccc(N(c3ccc(-c4ccc5ccccc5c4)cc3)c3ccc(-c4ccc5c(c4)oc4ccc6nc(-c7ccccc7)oc6c45)cc3)cc2)cc1]

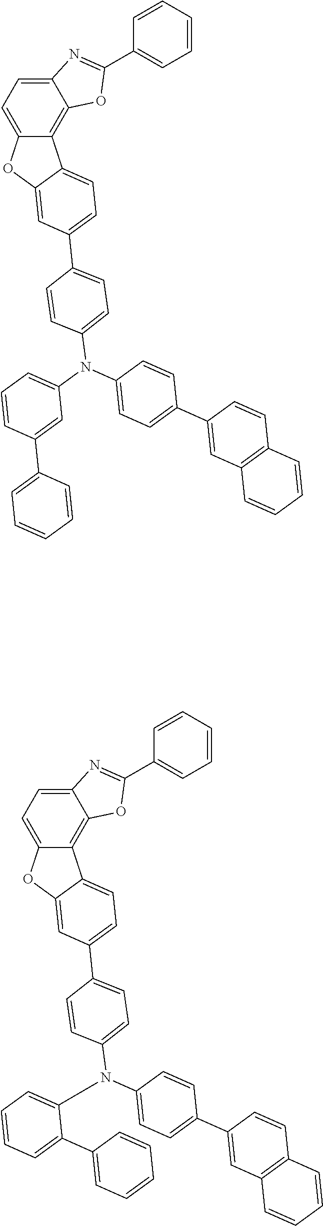 [SMILES: c1ccc(-c2cccc(N(c3ccc(-c4ccc5ccccc5c4)cc3)c3ccc(-c4ccc5c(c4)oc4ccc6nc(-c7ccccc7)oc6c45)cc3)c2)cc1 ; c1ccc(-c2nc3ccc4oc5cc(-c6ccc(N(c7ccc(-c8ccc9ccccc9c8)cc7)c7ccccc7-c7ccccc7)cc6)ccc5c4c3o2)cc1]

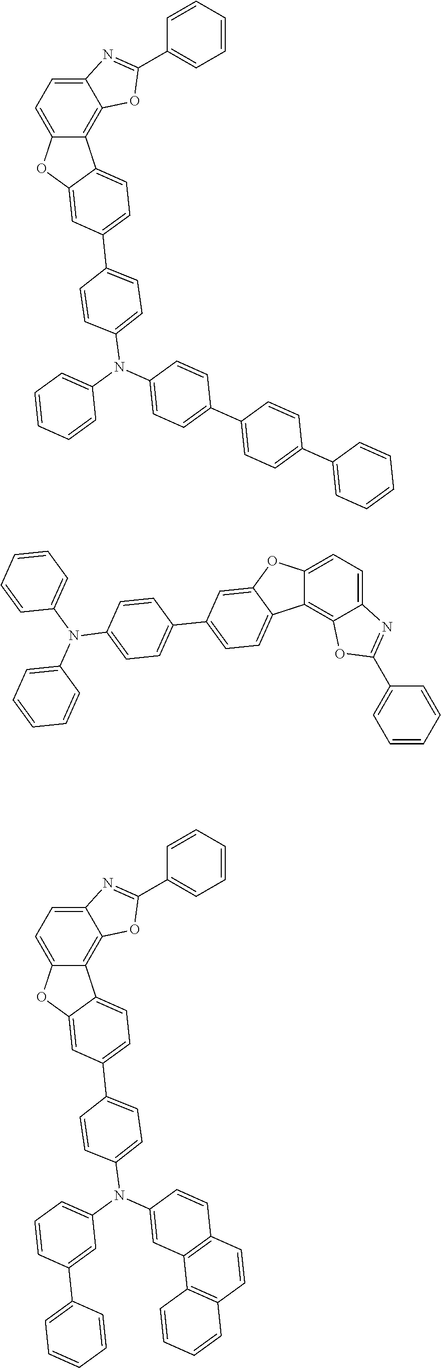 [SMILES: c1ccc(-c2ccc(-c3ccc(N(c4ccccc4)c4ccc(-c5ccc6c(c5)oc5ccc7nc(-c8ccccc8)oc7c56)cc4)cc3)cc2)cc1 ; c1ccc(-c2nc3ccc4oc5cc(-c6ccc(N(c7ccccc7)c7ccccc7)cc6)ccc5c4c3o2)cc1 ; c1ccc(-c2cccc(N(c3ccc(-c4ccc5c(c4)oc4ccc6nc(-c7ccccc7)oc6c45)cc3)c3ccc4ccc5ccccc5c4c3)c2)cc1]

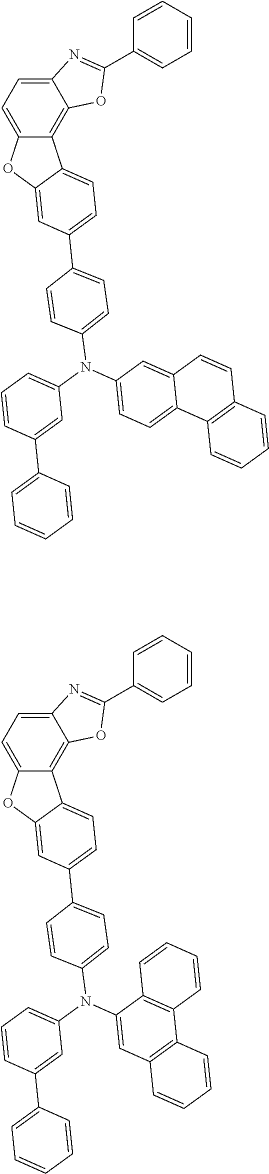 [SMILES: c1ccc(-c2cccc(N(c3ccc(-c4ccc5c(c4)oc4ccc6nc(-c7ccccc7)oc6c45)cc3)c3ccc4c(ccc5ccccc54)c3)c2)cc1 ; c1ccc(-c2cccc(N(c3ccc(-c4ccc5c(c4)oc4ccc6nc(-c7ccccc7)oc6c45)cc3)c3cc4ccccc4c4ccccc34)c2)cc1]

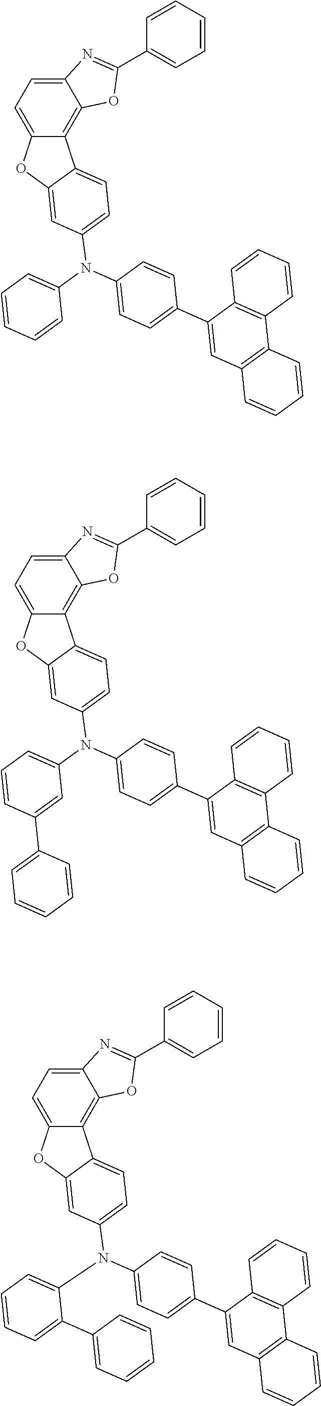 [SMILES: c1ccc(-c2nc3ccc4oc5cc(N(c6ccccc6)c6ccc(-c7cc8ccccc8c8ccccc78)cc6)ccc5c4c3o2)cc1 ; c1ccc(-c2cccc(N(c3ccc(-c4cc5ccccc5c5ccccc45)cc3)c3ccc4c(c3)oc3ccc5nc(-c6ccccc6)oc5c34)c2)cc1 ; c1ccc(-c2nc3ccc4oc5cc(N(c6ccc(-c7cc8ccccc8c8ccccc78)cc6)c6ccccc6-c6ccccc6)ccc5c4c3o2)cc1]

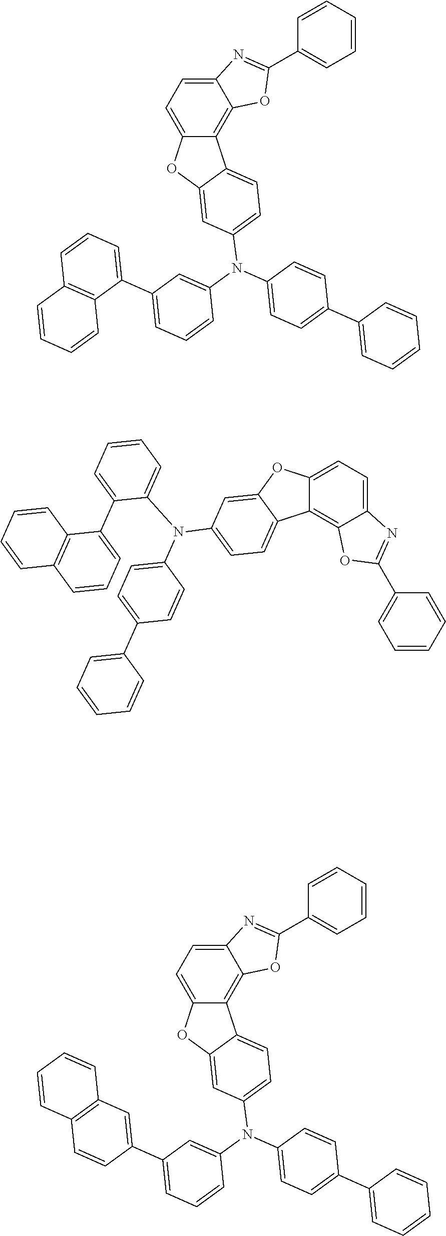 [SMILES: c1ccc(-c2ccc(N(c3cccc(-c4cccc5ccccc45)c3)c3ccc4c(c3)oc3ccc5nc(-c6ccccc6)oc5c34)cc2)cc1 ; c1ccc(-c2ccc(N(c3ccc4c(c3)oc3ccc5nc(-c6ccccc6)oc5c34)c3ccccc3-c3cccc4ccccc34)cc2)cc1 ; c1ccc(-c2ccc(N(c3cccc(-c4ccc5ccccc5c4)c3)c3ccc4c(c3)oc3ccc5nc(-c6ccccc6)oc5c34)cc2)cc1]

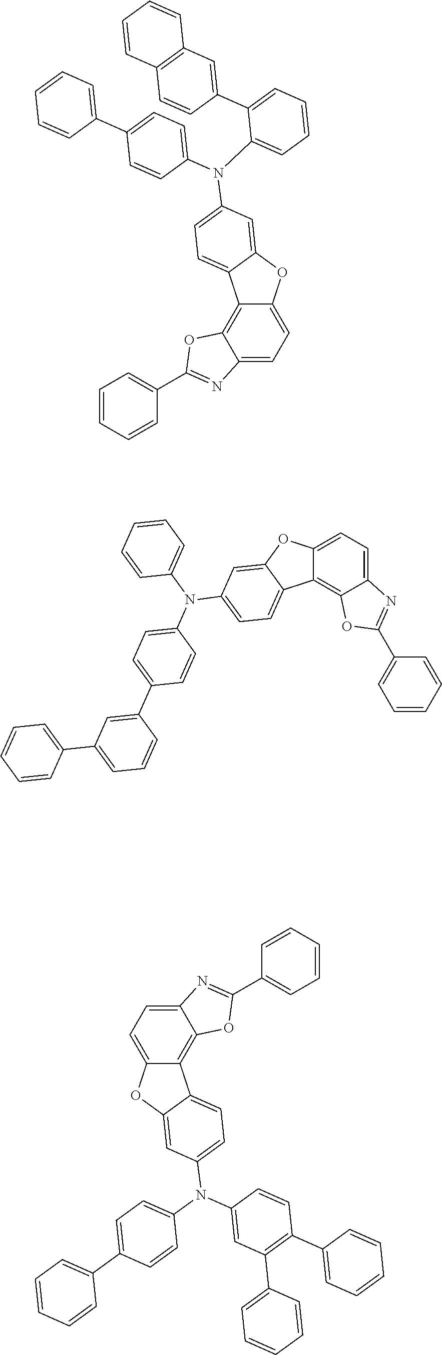 [SMILES: c1ccc(-c2ccc(N(c3ccc4c(c3)oc3ccc5nc(-c6ccccc6)oc5c34)c3ccccc3-c3ccc4ccccc4c3)cc2)cc1 ; c1ccc(-c2cccc(-c3ccc(N(c4ccccc4)c4ccc5c(c4)oc4ccc6nc(-c7ccccc7)oc6c45)cc3)c2)cc1 ; c1ccc(-c2ccc(N(c3ccc(-c4ccccc4)c(-c4ccccc4)c3)c3ccc4c(c3)oc3ccc5nc(-c6ccccc6)oc5c34)cc2)cc1]

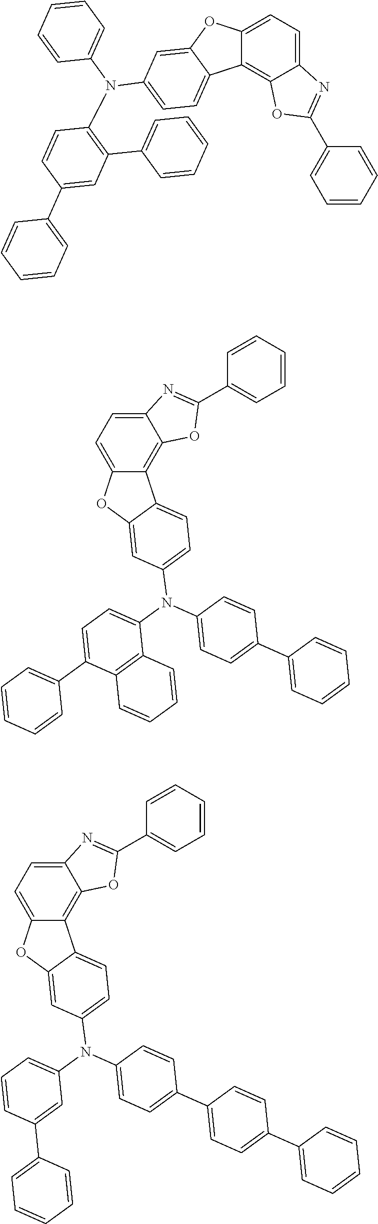 [SMILES: c1ccc(-c2ccc(N(c3ccccc3)c3ccc4c(c3)oc3ccc5nc(-c6ccccc6)oc5c34)c(-c3ccccc3)c2)cc1 ; c1ccc(-c2ccc(N(c3ccc4c(c3)oc3ccc5nc(-c6ccccc6)oc5c34)c3ccc(-c4ccccc4)c4ccccc34)cc2)cc1 ; c1ccc(-c2ccc(-c3ccc(N(c4cccc(-c5ccccc5)c4)c4ccc5c(c4)oc4ccc6nc(-c7ccccc7)oc6c45)cc3)cc2)cc1]

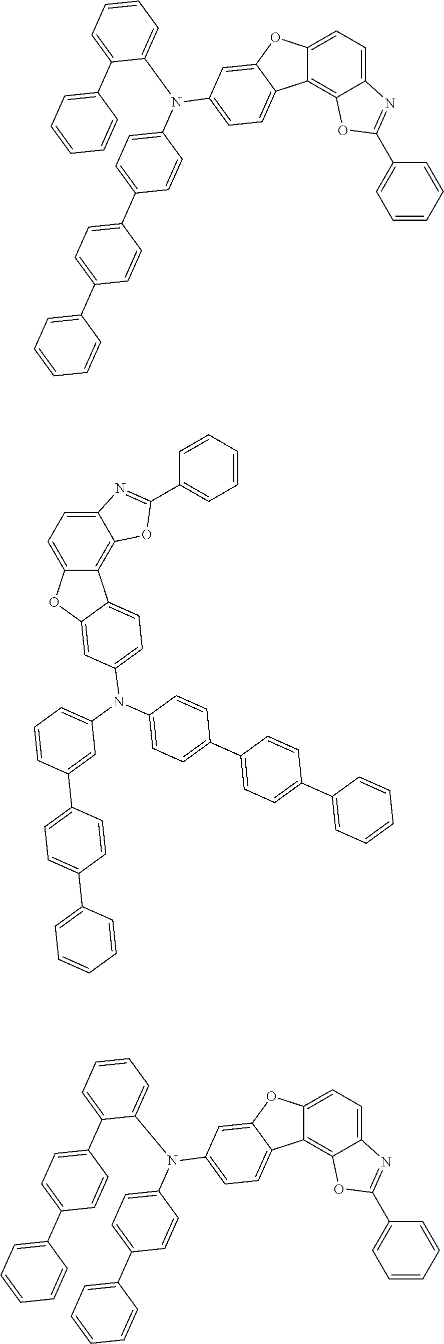 [SMILES: c1ccc(-c2ccc(-c3ccc(N(c4ccc5c(c4)oc4ccc6nc(-c7ccccc7)oc6c45)c4ccccc4-c4ccccc4)cc3)cc2)cc1 ; c1ccc(-c2ccc(-c3ccc(N(c4cccc(-c5ccc(-c6ccccc6)cc5)c4)c4ccc5c(c4)oc4ccc6nc(-c7ccccc7)oc6c45)cc3)cc2)cc1 ; c1ccc(-c2ccc(-c3ccccc3N(c3ccc(-c4ccccc4)cc3)c3ccc4c(c3)oc3ccc5nc(-c6ccccc6)oc5c34)cc2)cc1]

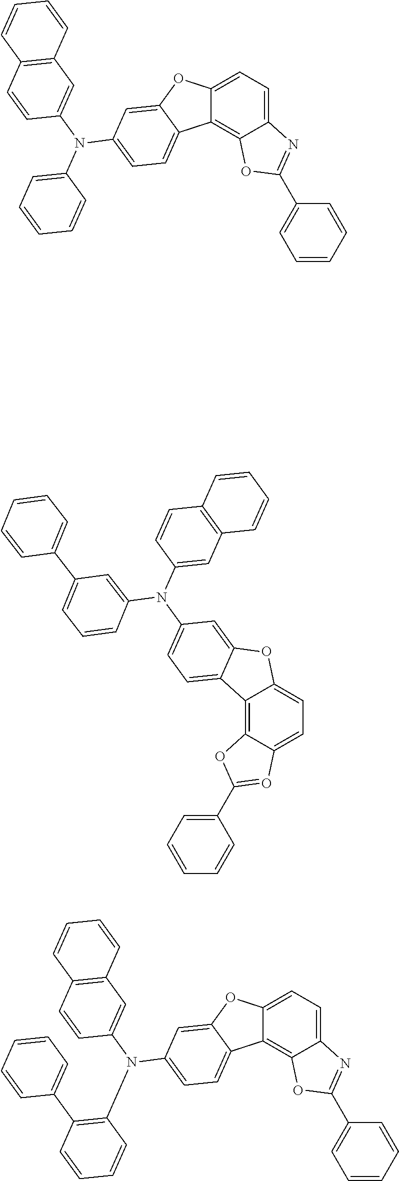

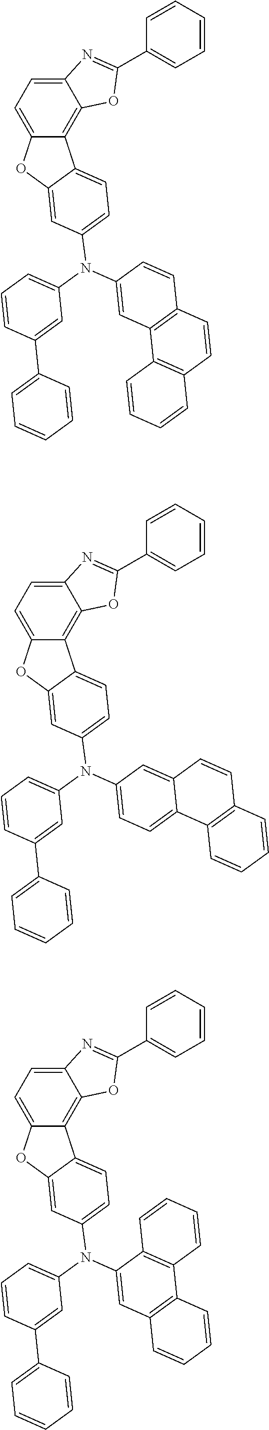 [SMILES: c1ccc(-c2cccc(N(c3ccc4c(c3)oc3ccc5nc(-c6ccccc6)oc5c34)c3ccc4ccc5ccccc5c4c3)c2)cc1 ; c1ccc(-c2cccc(N(c3ccc4c(ccc5ccccc54)c3)c3ccc4c(c3)oc3ccc5nc(-c6ccccc6)oc5c34)c2)cc1 ; c1ccc(-c2cccc(N(c3ccc4c(c3)oc3ccc5nc(-c6ccccc6)oc5c34)c3cc4ccccc4c4ccccc34)c2)cc1]

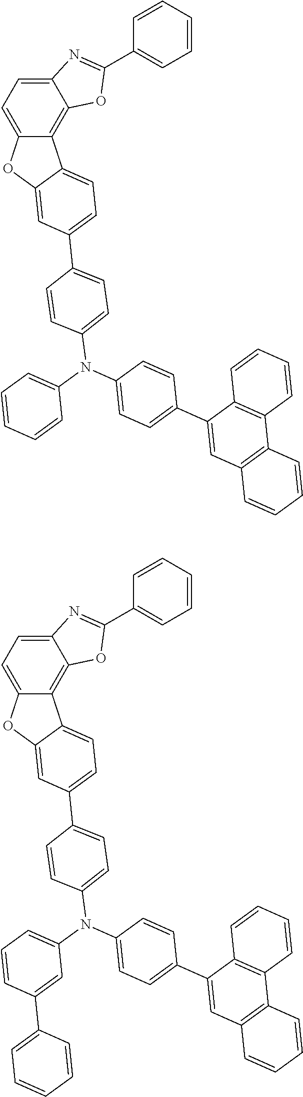 [SMILES: c1ccc(-c2nc3ccc4oc5cc(-c6ccc(N(c7ccccc7)c7ccc(-c8cc9ccccc9c9ccccc89)cc7)cc6)ccc5c4c3o2)cc1 ; c1ccc(-c2cccc(N(c3ccc(-c4ccc5c(c4)oc4ccc6nc(-c7ccccc7)oc6c45)cc3)c3ccc(-c4cc5ccccc5c5ccccc45)cc3)c2)cc1]

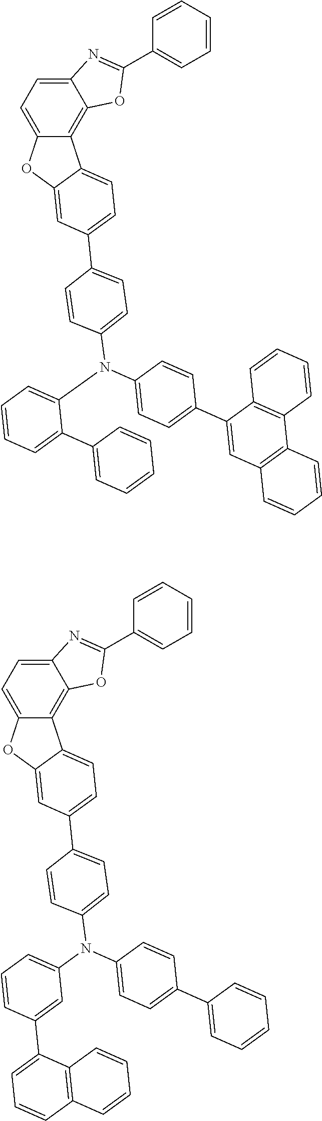 [SMILES: c1ccc(-c2nc3ccc4oc5cc(-c6ccc(N(c7ccc(-c8cc9ccccc9c9ccccc89)cc7)c7ccccc7-c7ccccc7)cc6)ccc5c4c3o2)cc1 ; c1ccc(-c2ccc(N(c3ccc(-c4ccc5c(c4)oc4ccc6nc(-c7ccccc7)oc6c45)cc3)c3cccc(-c4cccc5ccccc45)c3)cc2)cc1]

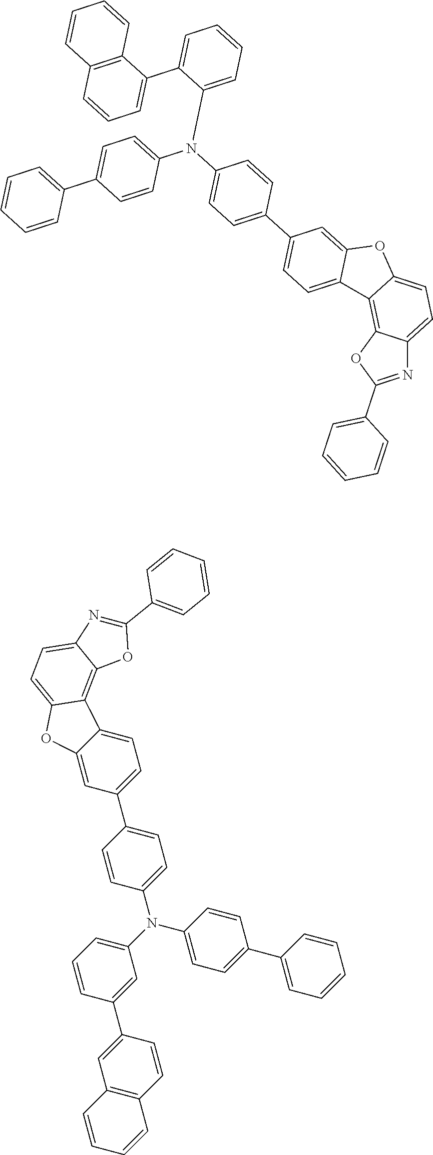 [SMILES: c1ccc(-c2ccc(N(c3ccc(-c4ccc5c(c4)oc4ccc6nc(-c7ccccc7)oc6c45)cc3)c3ccccc3-c3cccc4ccccc34)cc2)cc1 ; c1ccc(-c2ccc(N(c3ccc(-c4ccc5c(c4)oc4ccc6nc(-c7ccccc7)oc6c45)cc3)c3cccc(-c4ccc5ccccc5c4)c3)cc2)cc1]

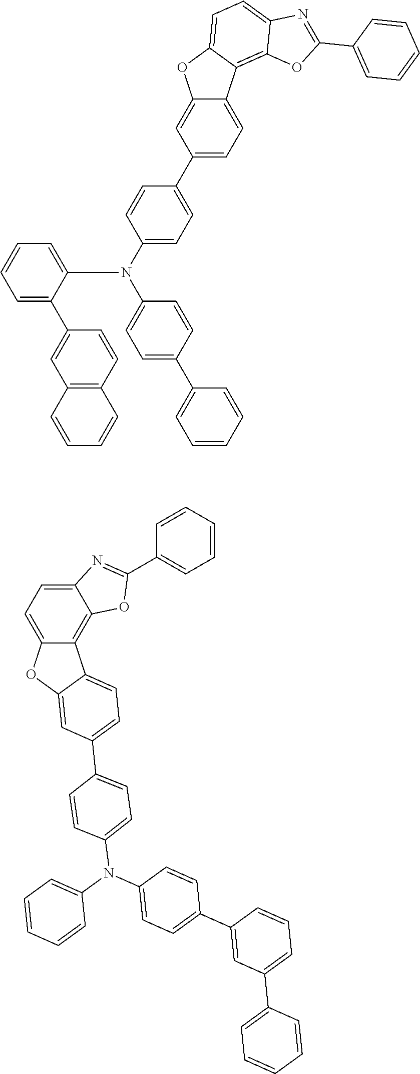 [SMILES: c1ccc(-c2ccc(N(c3ccc(-c4ccc5c(c4)oc4ccc6nc(-c7ccccc7)oc6c45)cc3)c3ccccc3-c3ccc4ccccc4c3)cc2)cc1 ; c1ccc(-c2cccc(-c3ccc(N(c4ccccc4)c4ccc(-c5ccc6c(c5)oc5ccc7nc(-c8ccccc8)oc7c56)cc4)cc3)c2)cc1]

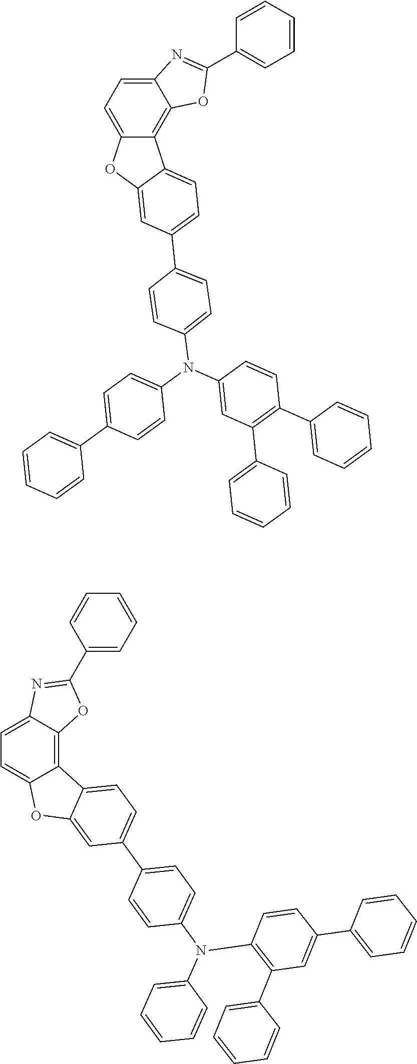 [SMILES: c1ccc(-c2ccc(N(c3ccc(-c4ccc5c(c4)oc4ccc6nc(-c7ccccc7)oc6c45)cc3)c3ccc(-c4ccccc4)c(-c4ccccc4)c3)cc2)cc1 ; c1ccc(-c2ccc(N(c3ccccc3)c3ccc(-c4ccc5c(c4)oc4ccc6nc(-c7ccccc7)oc6c45)cc3)c(-c3ccccc3)c2)cc1]

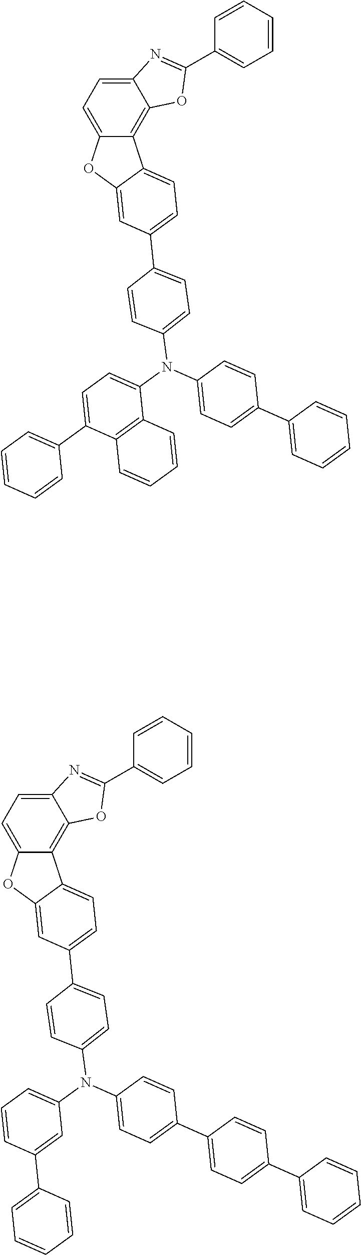 [SMILES: c1ccc(-c2ccc(N(c3ccc(-c4ccc5c(c4)oc4ccc6nc(-c7ccccc7)oc6c45)cc3)c3ccc(-c4ccccc4)c4ccccc34)cc2)cc1 ; c1ccc(-c2ccc(-c3ccc(N(c4ccc(-c5ccc6c(c5)oc5ccc7nc(-c8ccccc8)oc7c56)cc4)c4cccc(-c5ccccc5)c4)cc3)cc2)cc1]

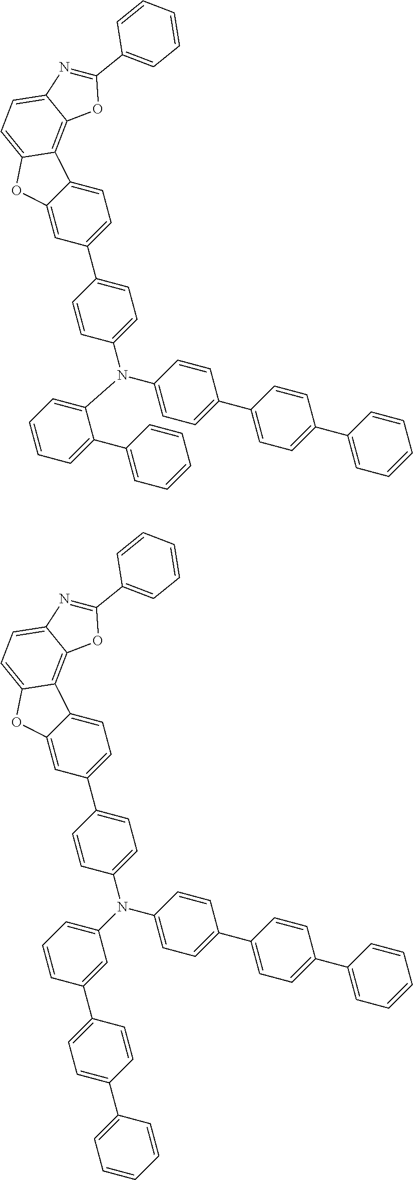 [SMILES: c1ccc(-c2ccc(-c3ccc(N(c4ccc(-c5ccc6c(c5)oc5ccc7nc(-c8ccccc8)oc7c56)cc4)c4ccccc4-c4ccccc4)cc3)cc2)cc1 ; c1ccc(-c2ccc(-c3ccc(N(c4ccc(-c5ccc6c(c5)oc5ccc7nc(-c8ccccc8)oc7c56)cc4)c4cccc(-c5ccc(-c6ccccc6)cc5)c4)cc3)cc2)cc1]

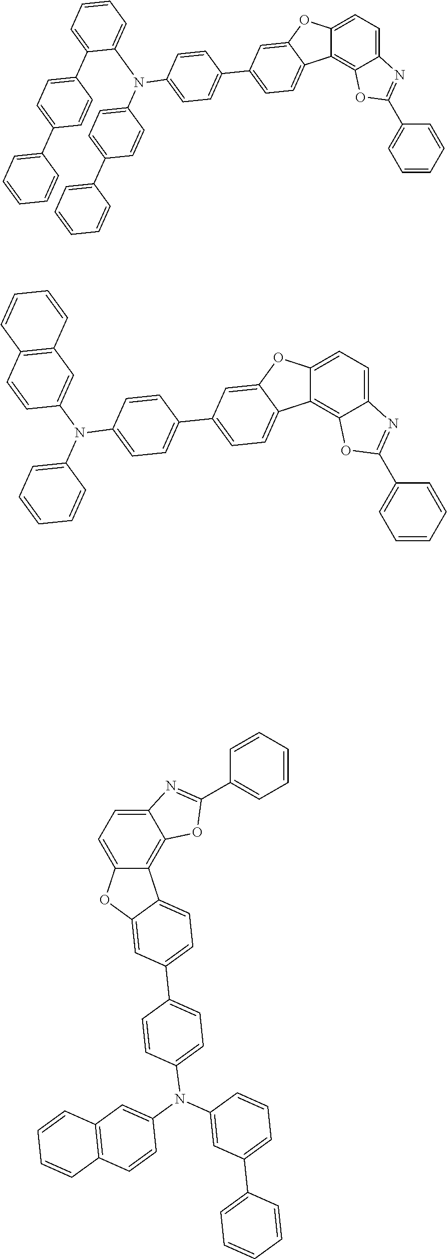 [SMILES: c1ccc(-c2ccc(-c3ccccc3N(c3ccc(-c4ccccc4)cc3)c3ccc(-c4ccc5c(c4)oc4ccc6nc(-c7ccccc7)oc6c45)cc3)cc2)cc1 ; c1ccc(-c2nc3ccc4oc5cc(-c6ccc(N(c7ccccc7)c7ccc8ccccc8c7)cc6)ccc5c4c3o2)cc1 ; c1ccc(-c2cccc(N(c3ccc(-c4ccc5c(c4)oc4ccc6nc(-c7ccccc7)oc6c45)cc3)c3ccc4ccccc4c3)c2)cc1]

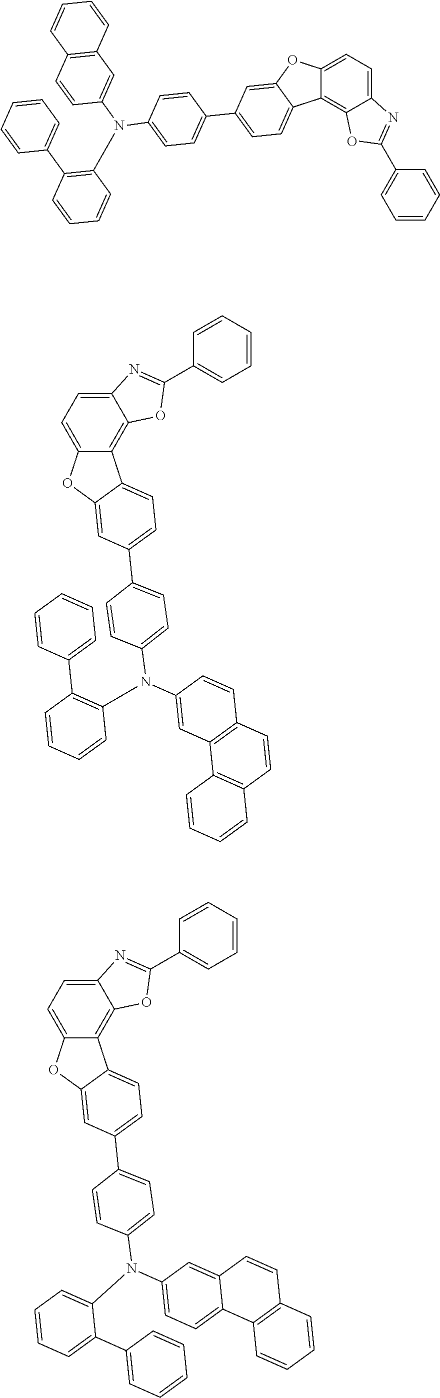 [SMILES: c1ccc(-c2nc3ccc4oc5cc(-c6ccc(N(c7ccc8ccccc8c7)c7ccccc7-c7ccccc7)cc6)ccc5c4c3o2)cc1 ; c1ccc(-c2nc3ccc4oc5cc(-c6ccc(N(c7ccc8ccc9ccccc9c8c7)c7ccccc7-c7ccccc7)cc6)ccc5c4c3o2)cc1 ; c1ccc(-c2nc3ccc4oc5cc(-c6ccc(N(c7ccc8c(ccc9ccccc98)c7)c7ccccc7-c7ccccc7)cc6)ccc5c4c3o2)cc1]

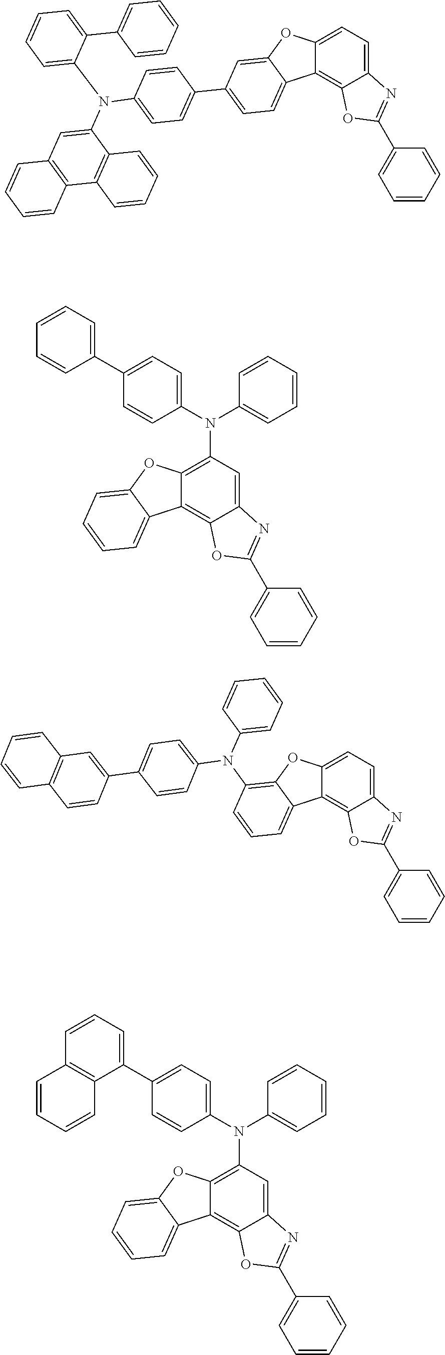

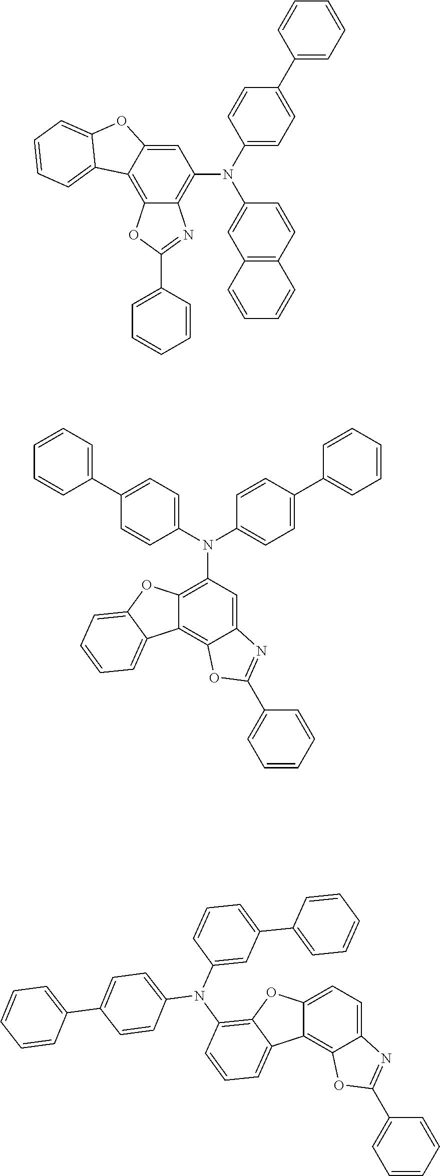 [SMILES: c1ccc(-c2ccc(N(c3ccc4ccccc4c3)c3cc4oc5ccccc5c4c4oc(-c5ccccc5)nc34)cc2)cc1 ; c1ccc(-c2ccc(N(c3ccc(-c4ccccc4)cc3)c3cc4nc(-c5ccccc5)oc4c4c3oc3ccccc34)cc2)cc1 ; c1ccc(-c2ccc(N(c3cccc(-c4ccccc4)c3)c3cccc4c3oc3ccc5nc(-c6ccccc6)oc5c34)cc2)cc1]

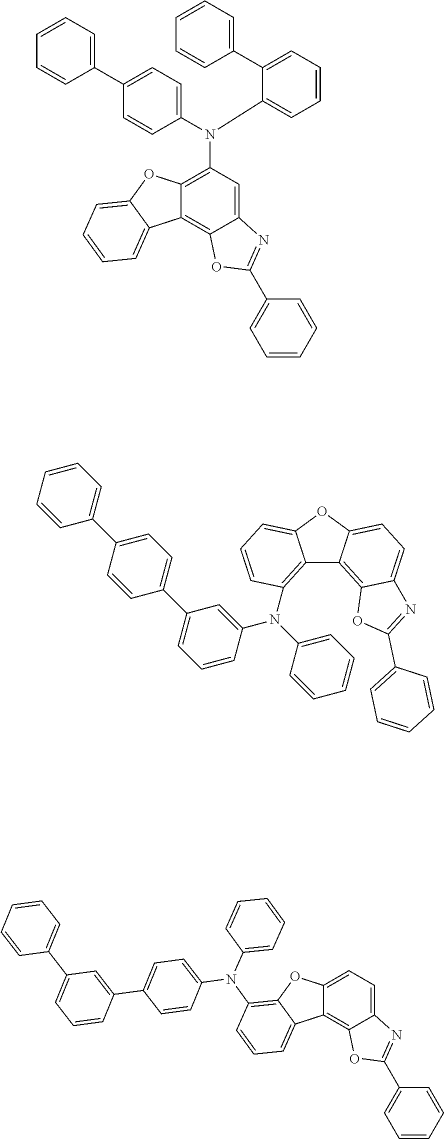 [SMILES: c1ccc(-c2ccc(N(c3ccccc3-c3ccccc3)c3cc4nc(-c5ccccc5)oc4c4c3oc3ccccc34)cc2)cc1 ; c1ccc(-c2ccc(-c3cccc(N(c4ccccc4)c4cccc5oc6ccc7nc(-c8ccccc8)oc7c6c45)c3)cc2)cc1 ; c1ccc(-c2cccc(-c3ccc(N(c4ccccc4)c4cccc5c4oc4ccc6nc(-c7ccccc7)oc6c45)cc3)c2)cc1]

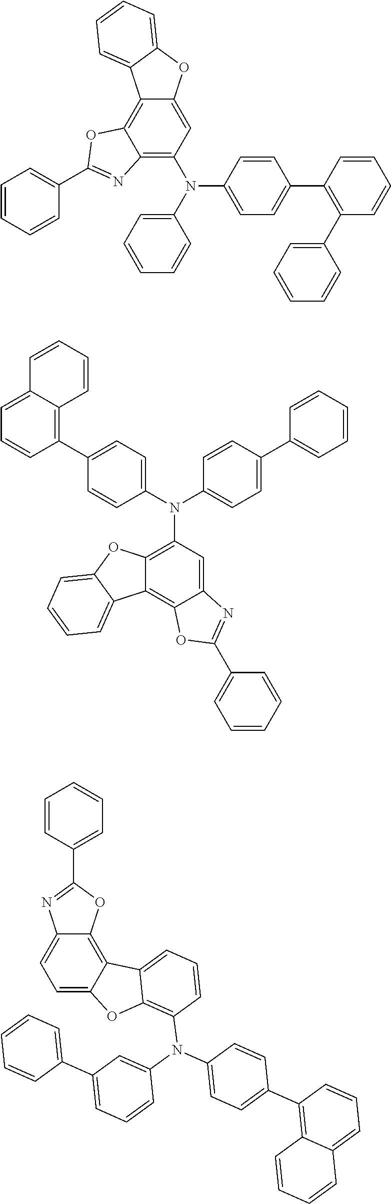 [SMILES: c1ccc(-c2nc3c(N(c4ccccc4)c4ccc(-c5ccccc5-c5ccccc5)cc4)cc4oc5ccccc5c4c3o2)cc1 ; c1ccc(-c2ccc(N(c3ccc(-c4cccc5ccccc45)cc3)c3cc4nc(-c5ccccc5)oc4c4c3oc3ccccc34)cc2)cc1 ; c1ccc(-c2cccc(N(c3ccc(-c4cccc5ccccc45)cc3)c3cccc4c3oc3ccc5nc(-c6ccccc6)oc5c34)c2)cc1]

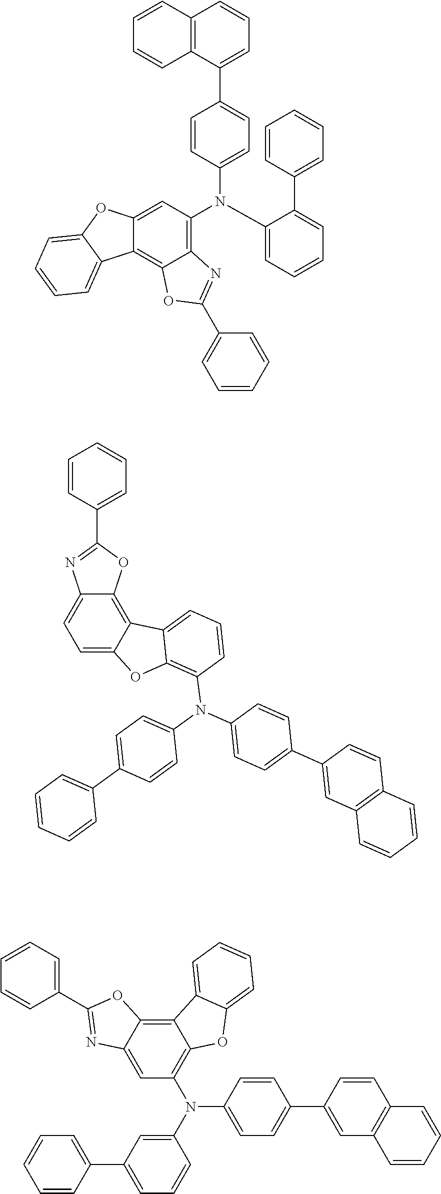 [SMILES: c1ccc(-c2nc3c(N(c4ccc(-c5cccc6ccccc56)cc4)c4ccccc4-c4ccccc4)cc4oc5ccccc5c4c3o2)cc1 ; c1ccc(-c2ccc(N(c3ccc(-c4ccc5ccccc5c4)cc3)c3cccc4c3oc3ccc5nc(-c6ccccc6)oc5c34)cc2)cc1 ; c1ccc(-c2cccc(N(c3ccc(-c4ccc5ccccc5c4)cc3)c3cc4nc(-c5ccccc5)oc4c4c3oc3ccccc34)c2)cc1]

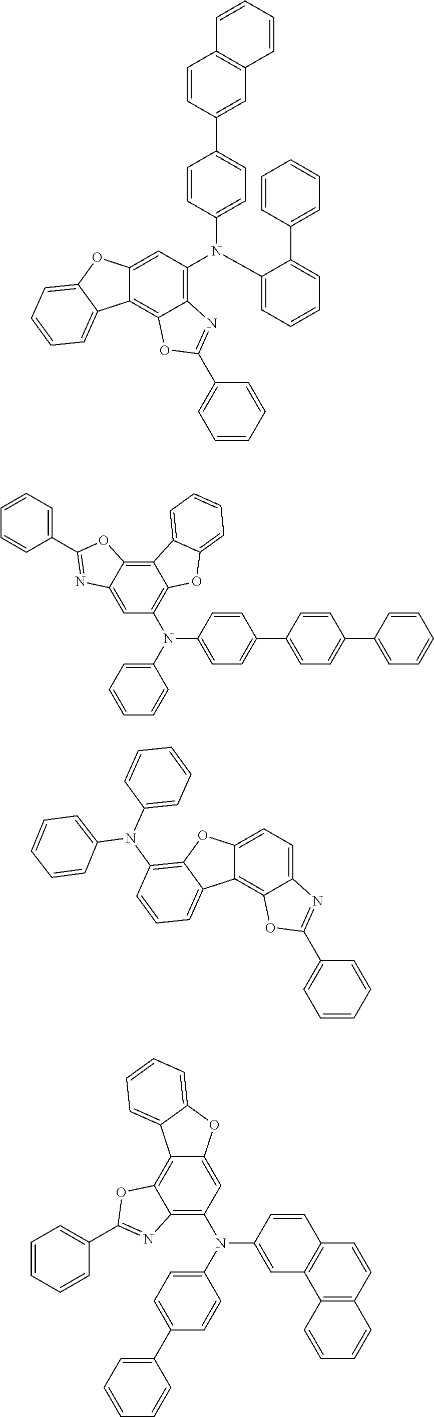

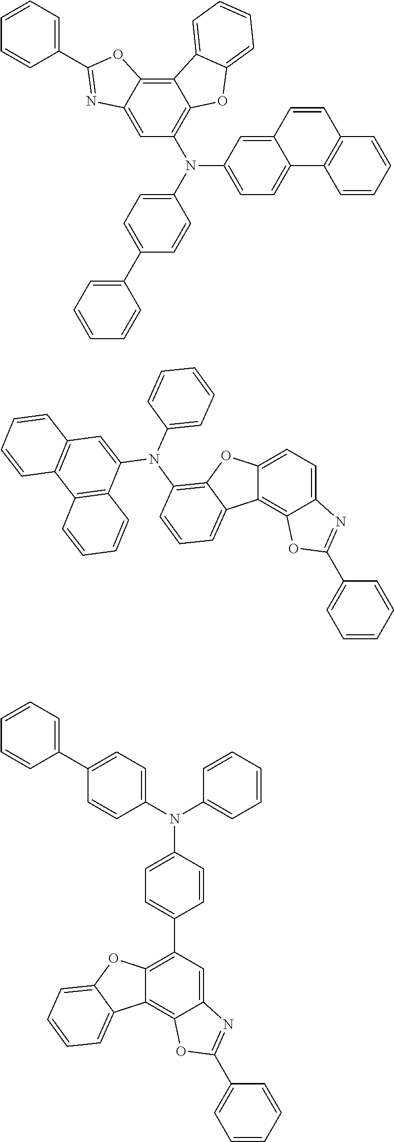 [SMILES: c1ccc(-c2ccc(N(c3ccc4c(ccc5ccccc54)c3)c3cc4nc(-c5ccccc5)oc4c4c3oc3ccccc34)cc2)cc1 ; c1ccc(-c2nc3ccc4oc5c(N(c6ccccc6)c6cc7ccccc7c7ccccc67)cccc5c4c3o2)cc1 ; c1ccc(-c2ccc(N(c3ccccc3)c3ccc(-c4cc5nc(-c6ccccc6)oc5c5c4oc4ccccc45)cc3)cc2)cc1]

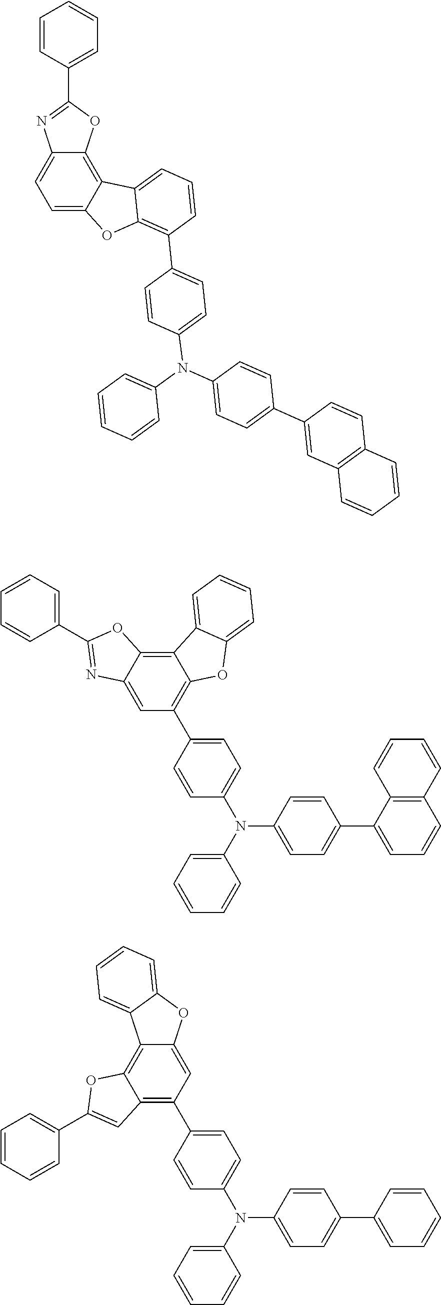 [SMILES: c1ccc(-c2nc3ccc4oc5c(-c6ccc(N(c7ccccc7)c7ccc(-c8ccc9ccccc9c8)cc7)cc6)cccc5c4c3o2)cc1 ; c1ccc(-c2nc3cc(-c4ccc(N(c5ccccc5)c5ccc(-c6cccc7ccccc67)cc5)cc4)c4oc5ccccc5c4c3o2)cc1 ; c1ccc(-c2ccc(N(c3ccccc3)c3ccc(-c4cc5oc6ccccc6c5c5oc(-c6ccccc6)cc45)cc3)cc2)cc1]

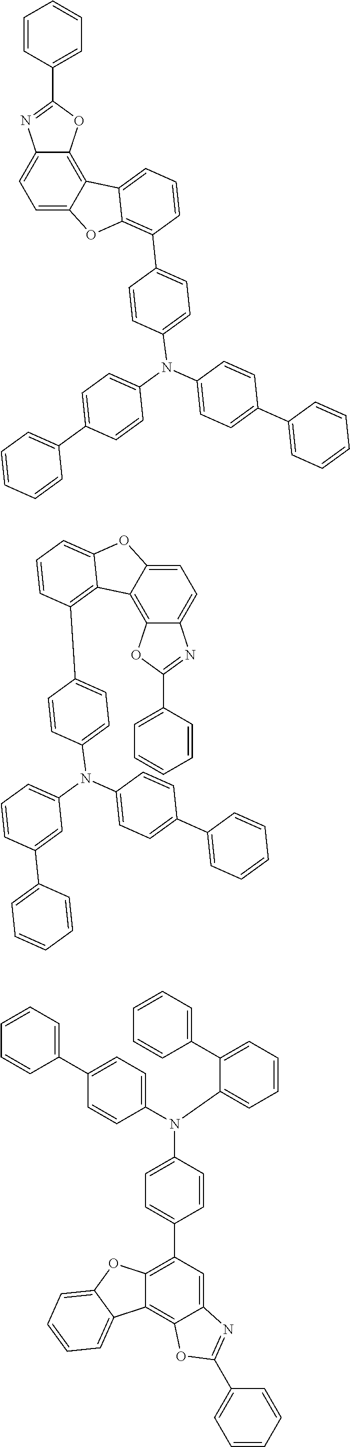 [SMILES: c1ccc(-c2ccc(N(c3ccc(-c4ccccc4)cc3)c3ccc(-c4cccc5c4oc4ccc6nc(-c7ccccc7)oc6c45)cc3)cc2)cc1 ; c1ccc(-c2ccc(N(c3ccc(-c4cccc5oc6ccc7nc(-c8ccccc8)oc7c6c45)cc3)c3cccc(-c4ccccc4)c3)cc2)cc1 ; c1ccc(-c2ccc(N(c3ccc(-c4cc5nc(-c6ccccc6)oc5c5c4oc4ccccc45)cc3)c3ccccc3-c3ccccc3)cc2)cc1]

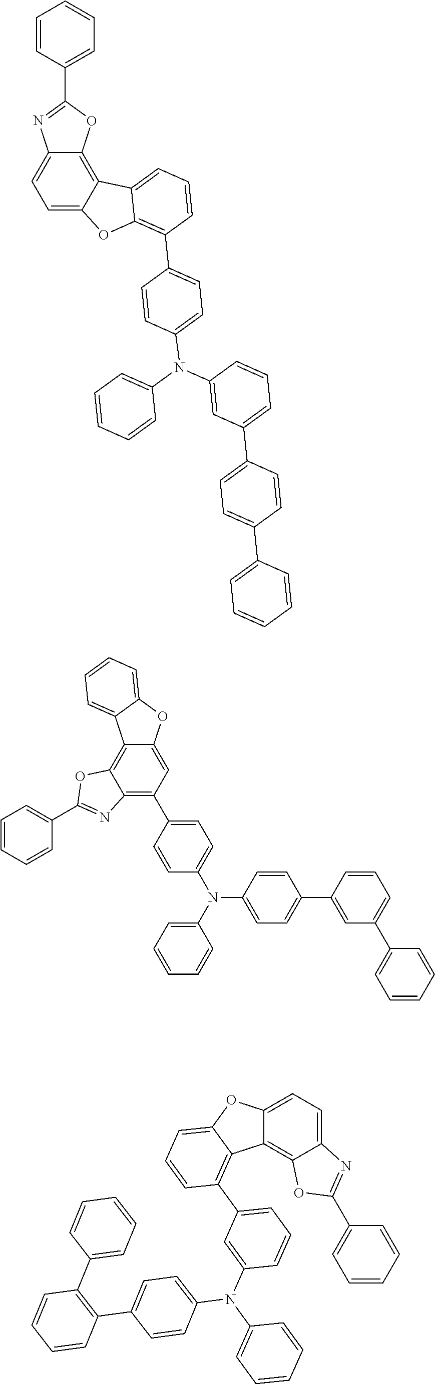 [SMILES: c1ccc(-c2ccc(-c3cccc(N(c4ccccc4)c4ccc(-c5cccc6c5oc5ccc7nc(-c8ccccc8)oc7c56)cc4)c3)cc2)cc1 ; c1ccc(-c2cccc(-c3ccc(N(c4ccccc4)c4ccc(-c5cc6oc7ccccc7c6c6oc(-c7ccccc7)nc56)cc4)cc3)c2)cc1 ; c1ccc(-c2nc3ccc4oc5cccc(-c6cccc(N(c7ccccc7)c7ccc(-c8ccccc8-c8ccccc8)cc7)c6)c5c4c3o2)cc1]

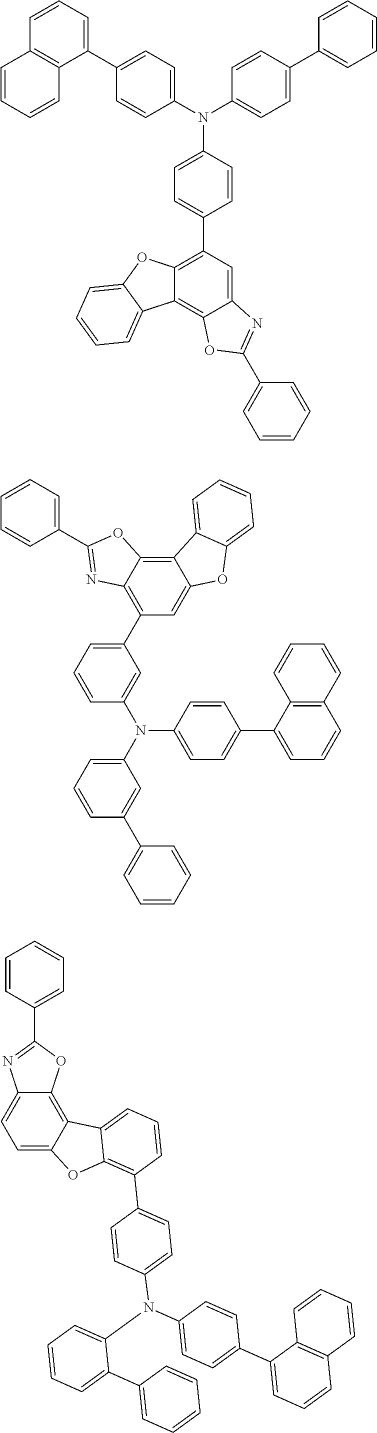 [SMILES: c1ccc(-c2ccc(N(c3ccc(-c4cccc5ccccc45)cc3)c3ccc(-c4cc5nc(-c6ccccc6)oc5c5c4oc4ccccc45)cc3)cc2)cc1 ; c1ccc(-c2cccc(N(c3ccc(-c4cccc5ccccc45)cc3)c3cccc(-c4cc5oc6ccccc6c5c5oc(-c6ccccc6)nc45)c3)c2)cc1 ; c1ccc(-c2nc3ccc4oc5c(-c6ccc(N(c7ccc(-c8cccc9ccccc89)cc7)c7ccccc7-c7ccccc7)cc6)cccc5c4c3o2)cc1]

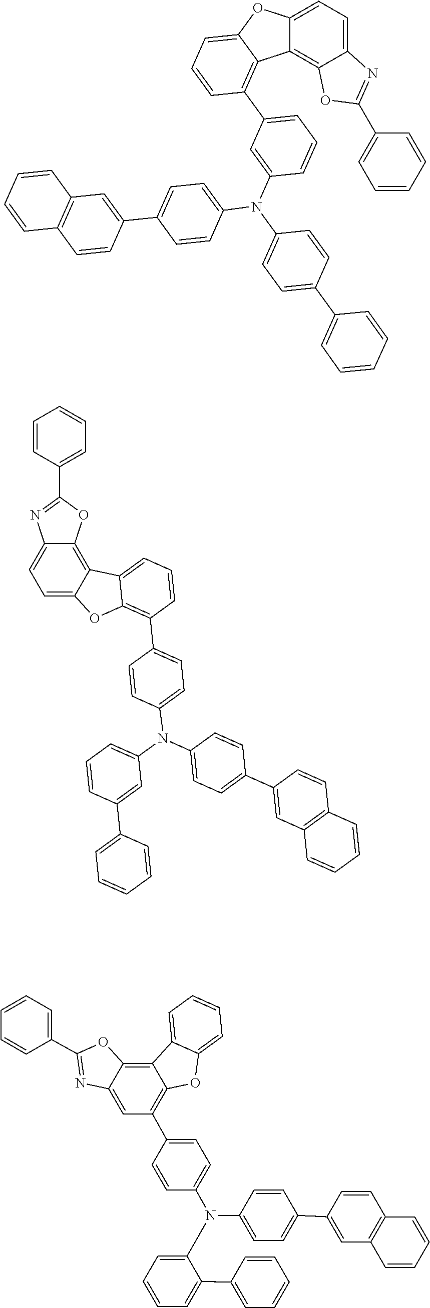 [SMILES: c1ccc(-c2ccc(N(c3ccc(-c4ccc5ccccc5c4)cc3)c3cccc(-c4cccc5oc6ccc7nc(-c8ccccc8)oc7c6c45)c3)cc2)cc1 ; c1ccc(-c2cccc(N(c3ccc(-c4ccc5ccccc5c4)cc3)c3ccc(-c4cccc5c4oc4ccc6nc(-c7ccccc7)oc6c45)cc3)c2)cc1 ; c1ccc(-c2nc3cc(-c4ccc(N(c5ccc(-c6ccc7ccccc7c6)cc5)c5ccccc5-c5ccccc5)cc4)c4oc5ccccc5c4c3o2)cc1]

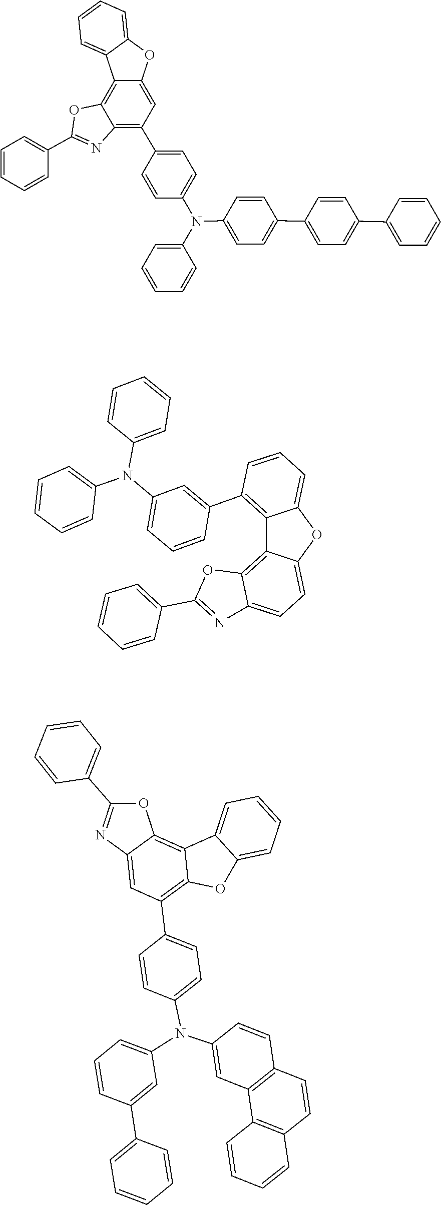 [SMILES: c1ccc(-c2ccc(-c3ccc(N(c4ccccc4)c4ccc(-c5cc6oc7ccccc7c6c6oc(-c7ccccc7)nc56)cc4)cc3)cc2)cc1 ; c1ccc(-c2nc3ccc4oc5cccc(-c6cccc(N(c7ccccc7)c7ccccc7)c6)c5c4c3o2)cc1 ; c1ccc(-c2cccc(N(c3ccc(-c4cc5nc(-c6ccccc6)oc5c5c4oc4ccccc45)cc3)c3ccc4ccc5ccccc5c4c3)c2)cc1]

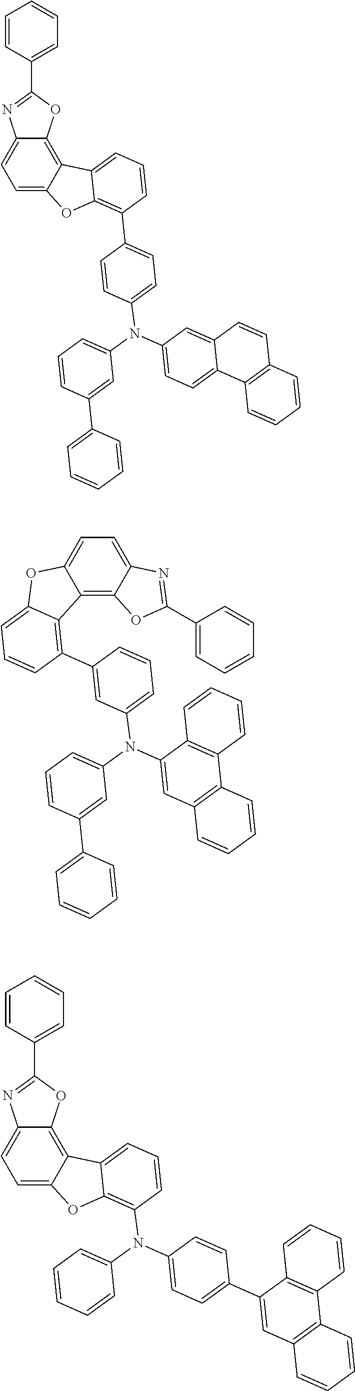 [SMILES: c1ccc(-c2cccc(N(c3ccc(-c4cccc5c4oc4ccc6nc(-c7ccccc7)oc6c45)cc3)c3ccc4c(ccc5ccccc54)c3)c2)cc1 ; c1ccc(-c2cccc(N(c3cccc(-c4cccc5oc6ccc7nc(-c8ccccc8)oc7c6c45)c3)c3cc4ccccc4c4ccccc34)c2)cc1 ; c1ccc(-c2nc3ccc4oc5c(N(c6ccccc6)c6ccc(-c7cc8ccccc8c8ccccc78)cc6)cccc5c4c3o2)cc1]

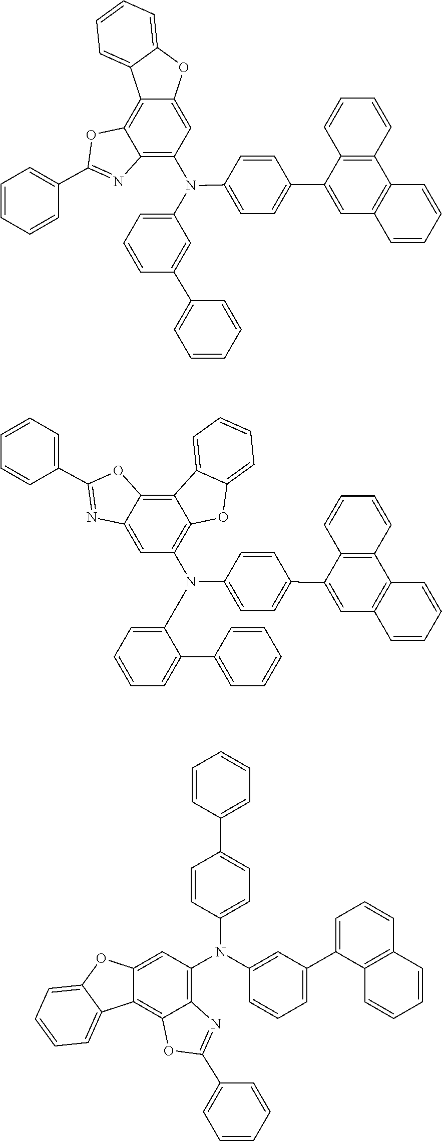 [SMILES: c1ccc(-c2cccc(N(c3ccc(-c4cc5ccccc5c5ccccc45)cc3)c3cc4oc5ccccc5c4c4oc(-c5ccccc5)nc34)c2)cc1 ; c1ccc(-c2nc3cc(N(c4ccc(-c5cc6ccccc6c6ccccc56)cc4)c4ccccc4-c4ccccc4)c4oc5ccccc5c4c3o2)cc1 ; c1ccc(-c2ccc(N(c3cccc(-c4cccc5ccccc45)c3)c3cc4oc5ccccc5c4c4oc(-c5ccccc5)nc34)cc2)cc1]

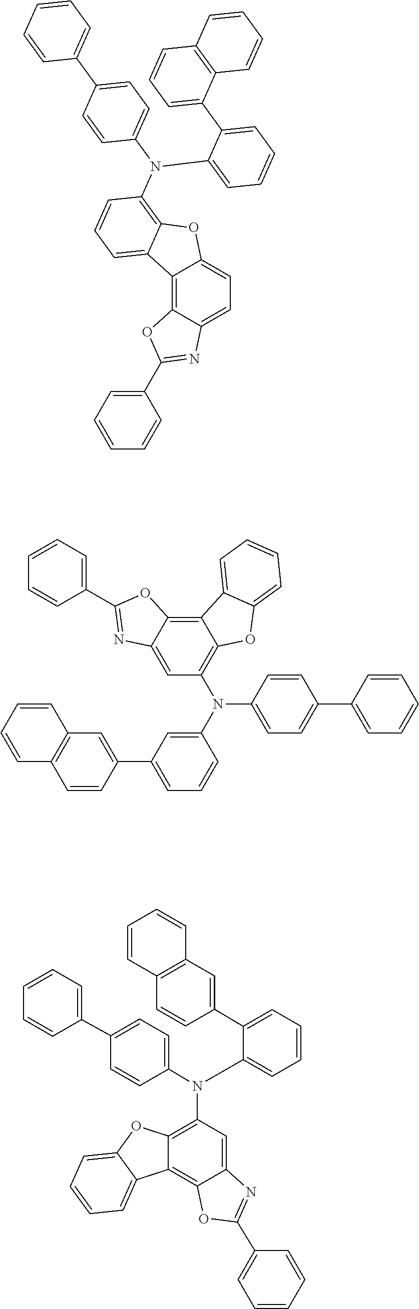 [SMILES: c1ccc(-c2ccc(N(c3ccccc3-c3cccc4ccccc34)c3cccc4c3oc3ccc5nc(-c6ccccc6)oc5c34)cc2)cc1 ; c1ccc(-c2ccc(N(c3cccc(-c4ccc5ccccc5c4)c3)c3cc4nc(-c5ccccc5)oc4c4c3oc3ccccc34)cc2)cc1 ; c1ccc(-c2ccc(N(c3ccccc3-c3ccc4ccccc4c3)c3cc4nc(-c5ccccc5)oc4c4c3oc3ccccc34)cc2)cc1]

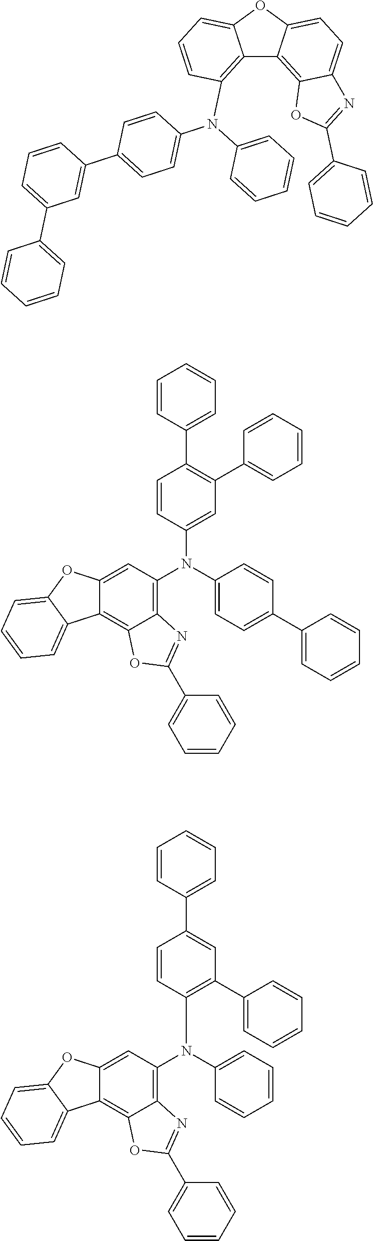 [SMILES: c1ccc(-c2cccc(-c3ccc(N(c4ccccc4)c4cccc5oc6ccc7nc(-c8ccccc8)oc7c6c45)cc3)c2)cc1 ; c1ccc(-c2ccc(N(c3ccc(-c4ccccc4)c(-c4ccccc4)c3)c3cc4oc5ccccc5c4c4oc(-c5ccccc5)nc34)cc2)cc1 ; c1ccc(-c2ccc(N(c3ccccc3)c3cc4oc5ccccc5c4c4oc(-c5ccccc5)nc34)c(-c3ccccc3)c2)cc1]

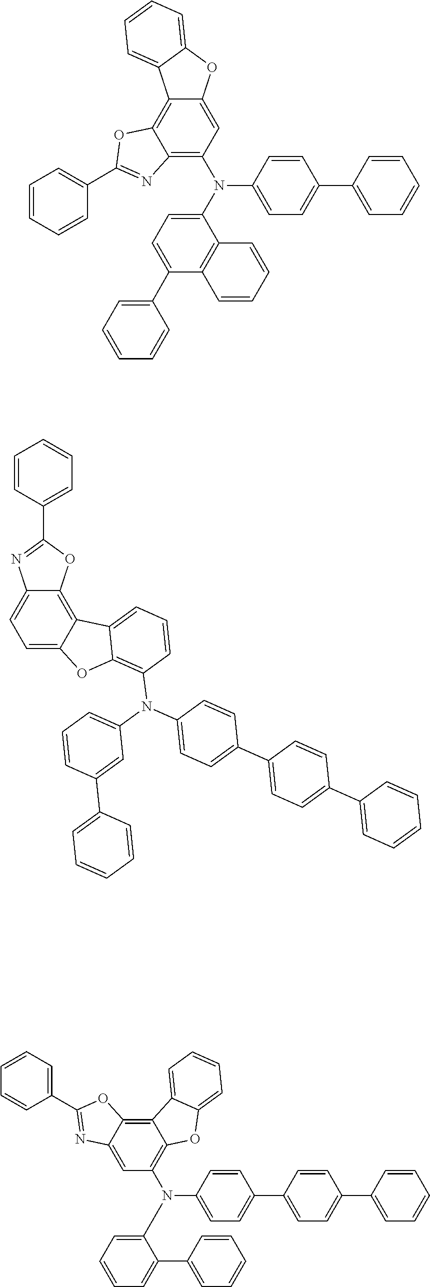 [SMILES: c1ccc(-c2ccc(N(c3ccc(-c4ccccc4)c4ccccc34)c3cc4oc5ccccc5c4c4oc(-c5ccccc5)nc34)cc2)cc1 ; c1ccc(-c2ccc(-c3ccc(N(c4cccc(-c5ccccc5)c4)c4cccc5c4oc4ccc6nc(-c7ccccc7)oc6c45)cc3)cc2)cc1 ; c1ccc(-c2ccc(-c3ccc(N(c4ccccc4-c4ccccc4)c4cc5nc(-c6ccccc6)oc5c5c4oc4ccccc45)cc3)cc2)cc1]

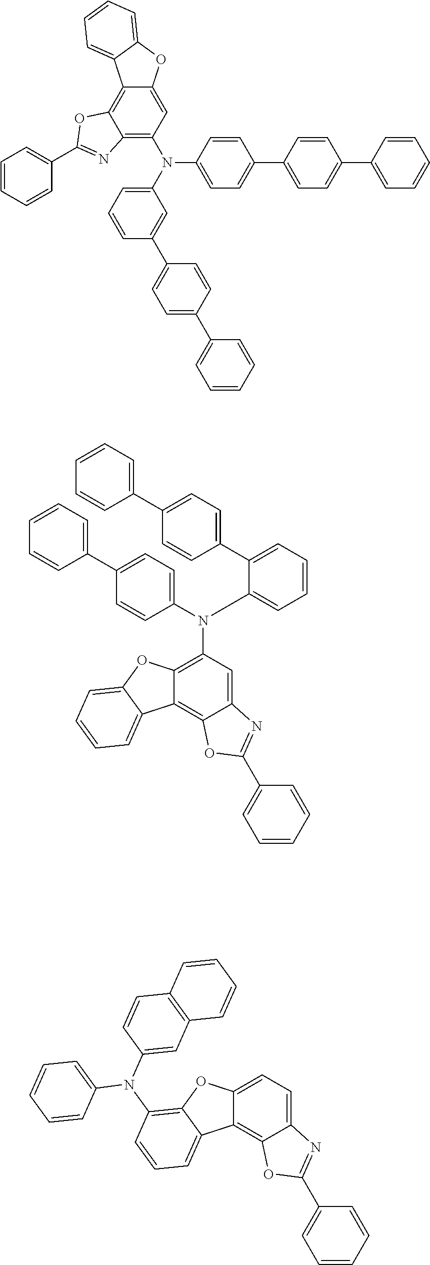 [SMILES: c1ccc(-c2ccc(-c3ccc(N(c4cccc(-c5ccc(-c6ccccc6)cc5)c4)c4cc5oc6ccccc6c5c5oc(-c6ccccc6)nc45)cc3)cc2)cc1 ; c1ccc(-c2ccc(-c3ccccc3N(c3ccc(-c4ccccc4)cc3)c3cc4nc(-c5ccccc5)oc4c4c3oc3ccccc34)cc2)cc1 ; c1ccc(-c2nc3ccc4oc5c(N(c6ccccc6)c6ccc7ccccc7c6)cccc5c4c3o2)cc1]

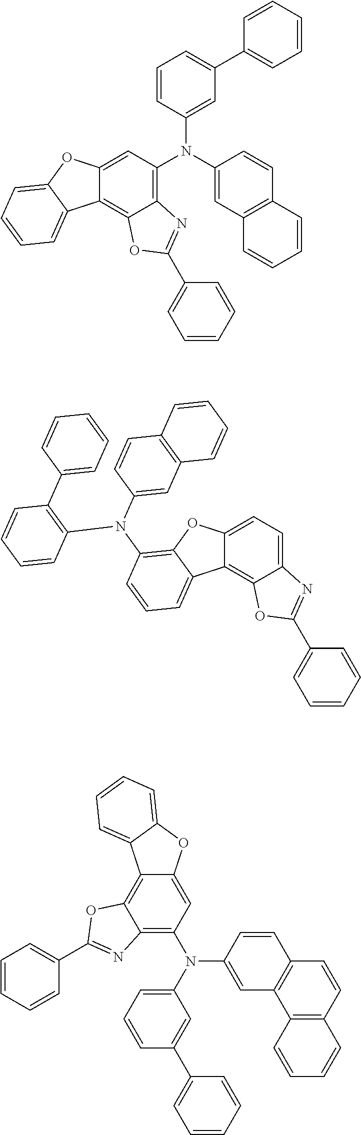 [SMILES: c1ccc(-c2cccc(N(c3ccc4ccccc4c3)c3cc4oc5ccccc5c4c4oc(-c5ccccc5)nc34)c2)cc1 ; c1ccc(-c2nc3ccc4oc5c(N(c6ccc7ccccc7c6)c6ccccc6-c6ccccc6)cccc5c4c3o2)cc1 ; c1ccc(-c2cccc(N(c3ccc4ccc5ccccc5c4c3)c3cc4oc5ccccc5c4c4oc(-c5ccccc5)nc34)c2)cc1]

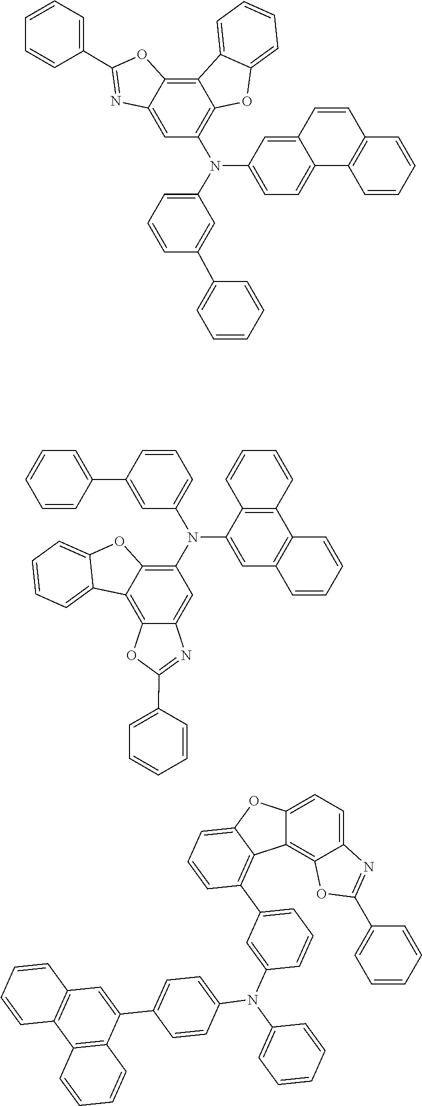 [SMILES: c1ccc(-c2cccc(N(c3ccc4c(ccc5ccccc54)c3)c3cc4nc(-c5ccccc5)oc4c4c3oc3ccccc34)c2)cc1 ; c1ccc(-c2cccc(N(c3cc4ccccc4c4ccccc34)c3cc4nc(-c5ccccc5)oc4c4c3oc3ccccc34)c2)cc1 ; c1ccc(-c2nc3ccc4oc5cccc(-c6cccc(N(c7ccccc7)c7ccc(-c8cc9ccccc9c9ccccc89)cc7)c6)c5c4c3o2)cc1]

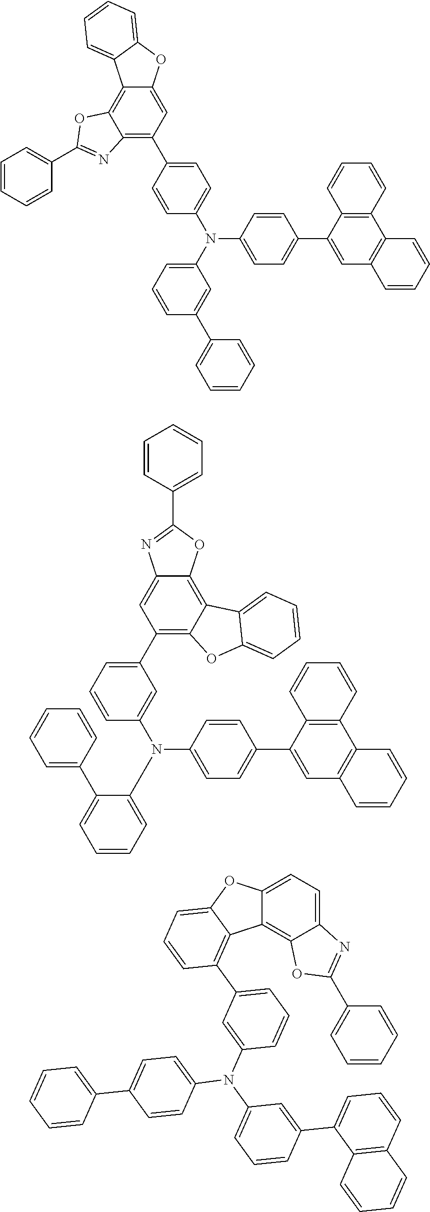 [SMILES: c1ccc(-c2cccc(N(c3ccc(-c4cc5ccccc5c5ccccc45)cc3)c3ccc(-c4cc5oc6ccccc6c5c5oc(-c6ccccc6)nc45)cc3)c2)cc1 ; c1ccc(-c2nc3cc(-c4cccc(N(c5ccc(-c6cc7ccccc7c7ccccc67)cc5)c5ccccc5-c5ccccc5)c4)c4oc5ccccc5c4c3o2)cc1 ; c1ccc(-c2ccc(N(c3cccc(-c4cccc5ccccc45)c3)c3cccc(-c4cccc5oc6ccc7nc(-c8ccccc8)oc7c6c45)c3)cc2)cc1]

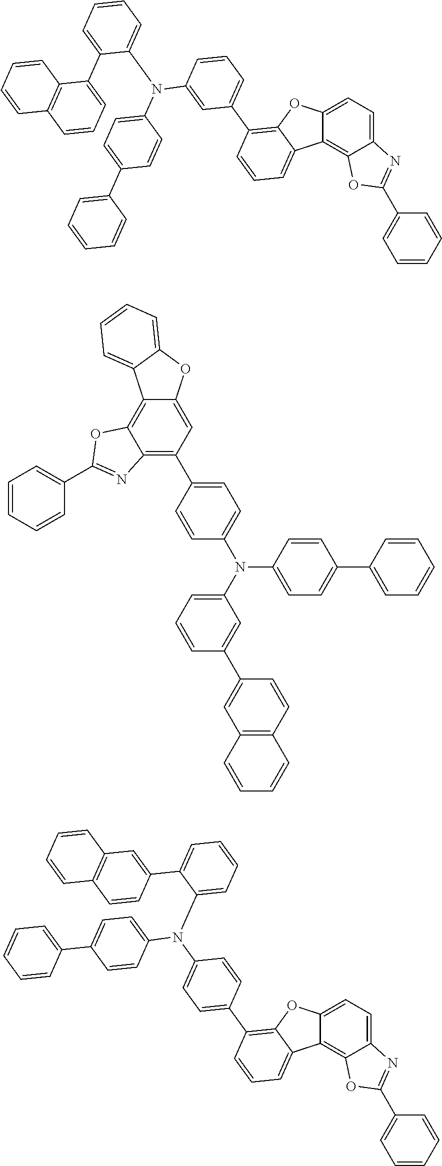 [SMILES: c1ccc(-c2ccc(N(c3cccc(-c4cccc5c4oc4ccc6nc(-c7ccccc7)oc6c45)c3)c3ccccc3-c3cccc4ccccc34)cc2)cc1 ; c1ccc(-c2ccc(N(c3ccc(-c4cc5oc6ccccc6c5c5oc(-c6ccccc6)nc45)cc3)c3cccc(-c4ccc5ccccc5c4)c3)cc2)cc1 ; c1ccc(-c2ccc(N(c3ccc(-c4cccc5c4oc4ccc6nc(-c7ccccc7)oc6c45)cc3)c3ccccc3-c3ccc4ccccc4c3)cc2)cc1]

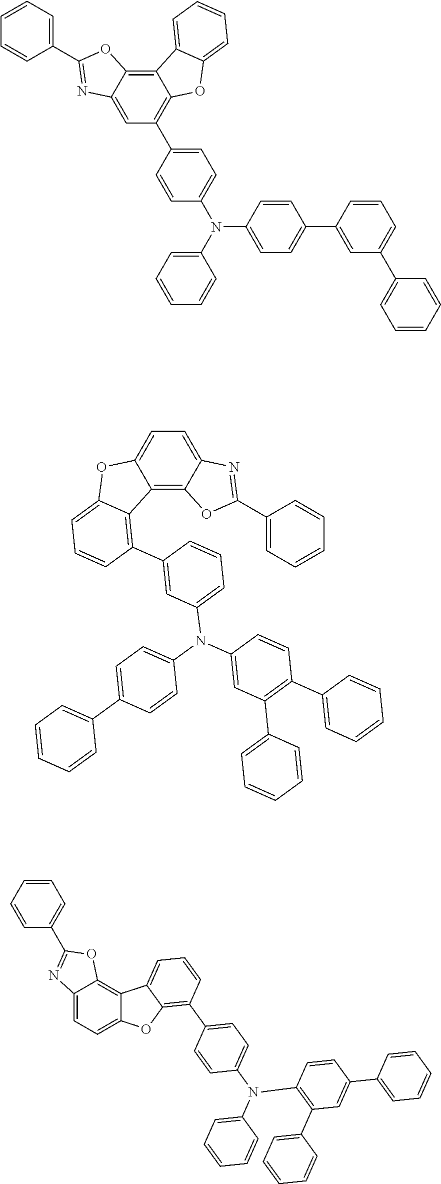 [SMILES: c1ccc(-c2cccc(-c3ccc(N(c4ccccc4)c4ccc(-c5cc6nc(-c7ccccc7)oc6c6c5oc5ccccc56)cc4)cc3)c2)cc1 ; c1ccc(-c2ccc(N(c3cccc(-c4cccc5oc6ccc7nc(-c8ccccc8)oc7c6c45)c3)c3ccc(-c4ccccc4)c(-c4ccccc4)c3)cc2)cc1 ; c1ccc(-c2ccc(N(c3ccccc3)c3ccc(-c4cccc5c4oc4ccc6nc(-c7ccccc7)oc6c45)cc3)c(-c3ccccc3)c2)cc1]

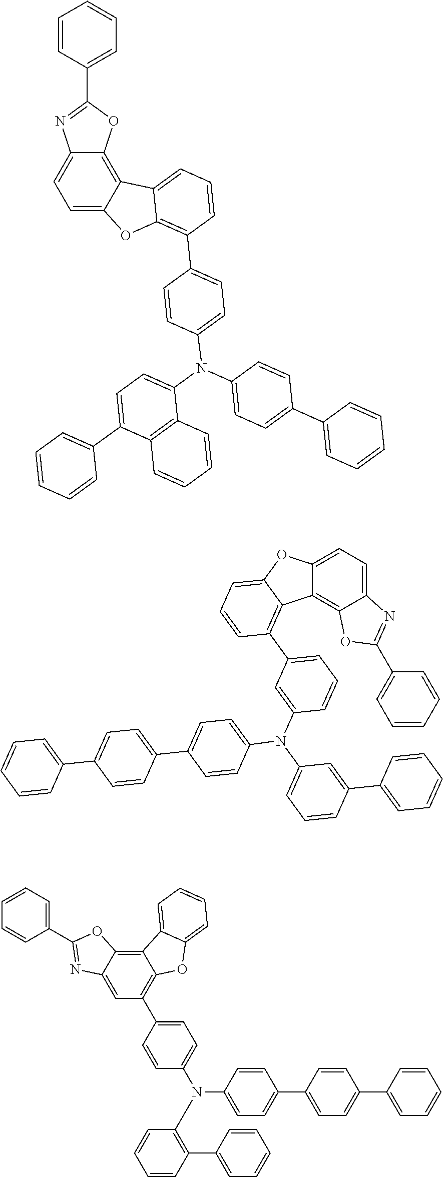 [SMILES: c1ccc(-c2ccc(N(c3ccc(-c4cccc5c4oc4ccc6nc(-c7ccccc7)oc6c45)cc3)c3ccc(-c4ccccc4)c4ccccc34)cc2)cc1 ; c1ccc(-c2ccc(-c3ccc(N(c4cccc(-c5ccccc5)c4)c4cccc(-c5cccc6oc7ccc8nc(-c9ccccc9)oc8c7c56)c4)cc3)cc2)cc1 ; c1ccc(-c2ccc(-c3ccc(N(c4ccc(-c5cc6nc(-c7ccccc7)oc6c6c5oc5ccccc56)cc4)c4ccccc4-c4ccccc4)cc3)cc2)cc1]

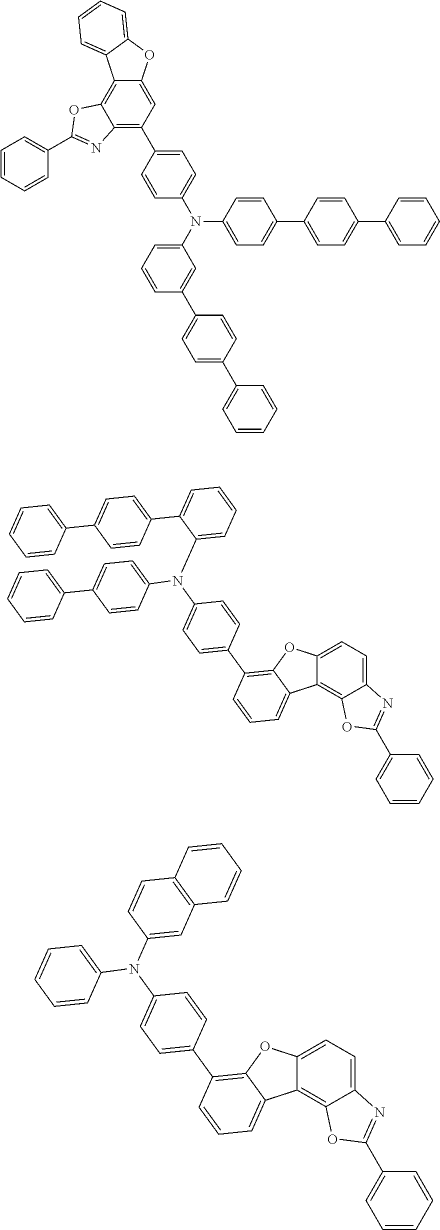 [SMILES: c1ccc(-c2ccc(-c3ccc(N(c4ccc(-c5cc6oc7ccccc7c6c6oc(-c7ccccc7)nc56)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4cccc5c4oc4ccc6nc(-c7ccccc7)oc6c45)cc3)cc2)cc1 ; c1ccc(-c2nc3ccc4oc5c(-c6ccc(N(c7ccccc7)c7ccc8ccccc8c7)cc6)cccc5c4c3o2)cc1]

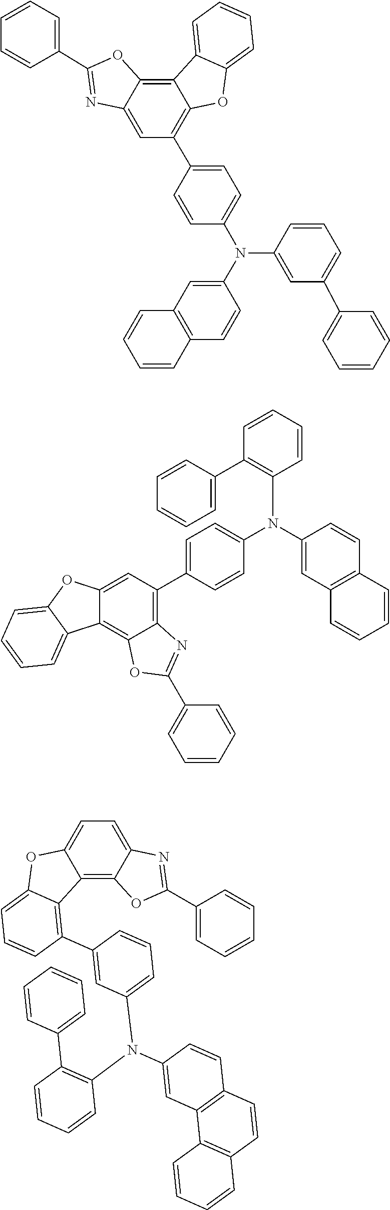 [SMILES: c1ccc(-c2cccc(N(c3ccc(-c4cc5nc(-c6ccccc6)oc5c5c4oc4ccccc45)cc3)c3ccc4ccccc4c3)c2)cc1 ; c1ccc(-c2nc3c(-c4ccc(N(c5ccc6ccccc6c5)c5ccccc5-c5ccccc5)cc4)cc4oc5ccccc5c4c3o2)cc1 ; c1ccc(-c2nc3ccc4oc5cccc(-c6cccc(N(c7ccc8ccc9ccccc9c8c7)c7ccccc7-c7ccccc7)c6)c5c4c3o2)cc1]

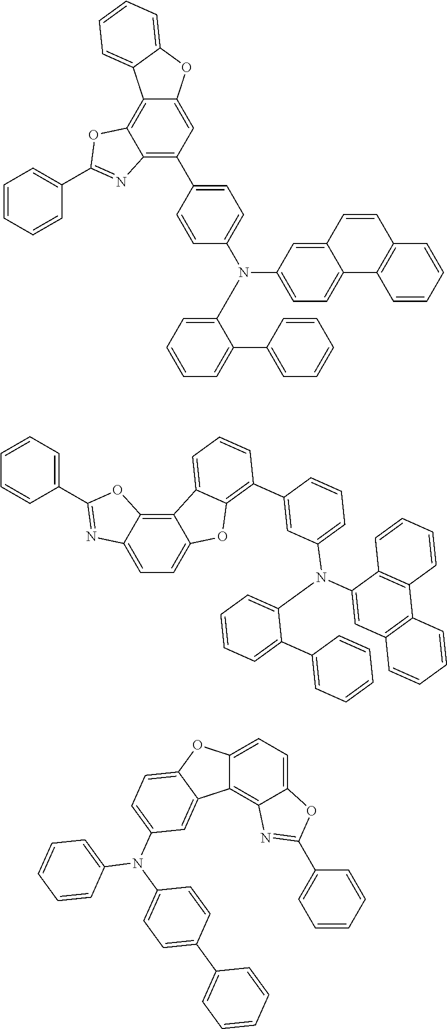 [SMILES: c1ccc(-c2nc3c(-c4ccc(N(c5ccc6c(ccc7ccccc76)c5)c5ccccc5-c5ccccc5)cc4)cc4oc5ccccc5c4c3o2)cc1 ; c1ccc(-c2nc3ccc4oc5c(-c6cccc(N(c7ccccc7-c7ccccc7)c7cc8ccccc8c8ccccc78)c6)cccc5c4c3o2)cc1 ; c1ccc(-c2ccc(N(c3ccccc3)c3ccc4oc5ccc6oc(-c7ccccc7)nc6c5c4c3)cc2)cc1]

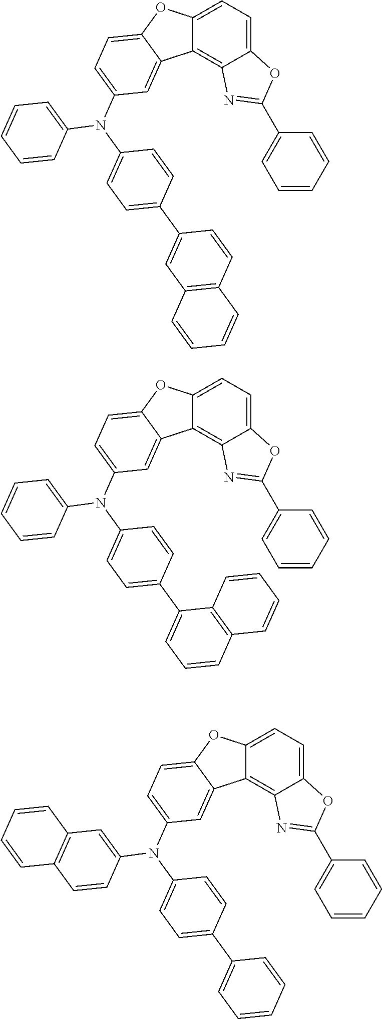

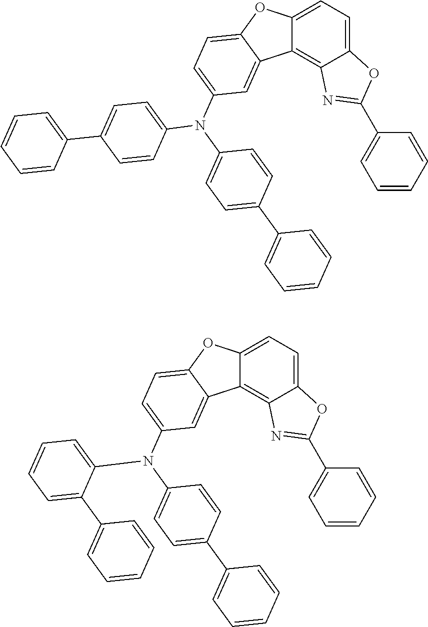 [SMILES: c1ccc(-c2ccc(N(c3ccc(-c4ccccc4)cc3)c3ccc4oc5ccc6oc(-c7ccccc7)nc6c5c4c3)cc2)cc1 ; c1ccc(-c2ccc(N(c3ccc4oc5ccc6oc(-c7ccccc7)nc6c5c4c3)c3ccccc3-c3ccccc3)cc2)cc1]

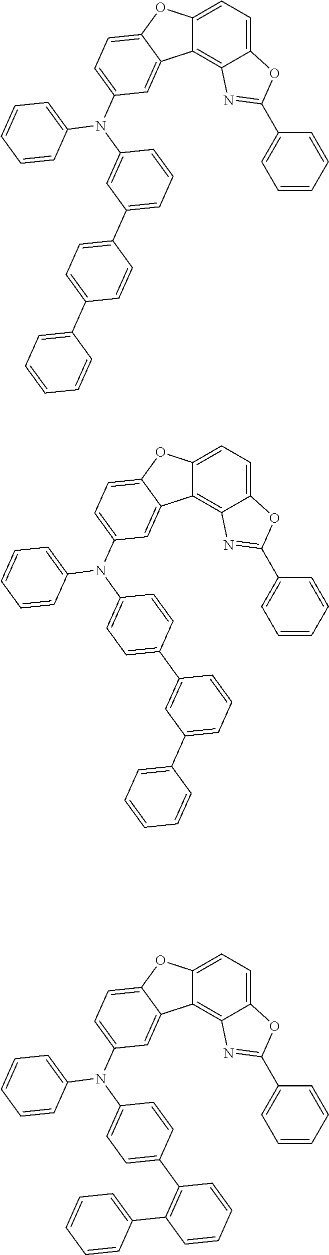 [SMILES: c1ccc(-c2ccc(-c3cccc(N(c4ccccc4)c4ccc5oc6ccc7oc(-c8ccccc8)nc7c6c5c4)c3)cc2)cc1 ; c1ccc(-c2cccc(-c3ccc(N(c4ccccc4)c4ccc5oc6ccc7oc(-c8ccccc8)nc7c6c5c4)cc3)c2)cc1 ; c1ccc(-c2nc3c(ccc4oc5ccc(N(c6ccccc6)c6ccc(-c7ccccc7-c7ccccc7)cc6)cc5c43)o2)cc1]

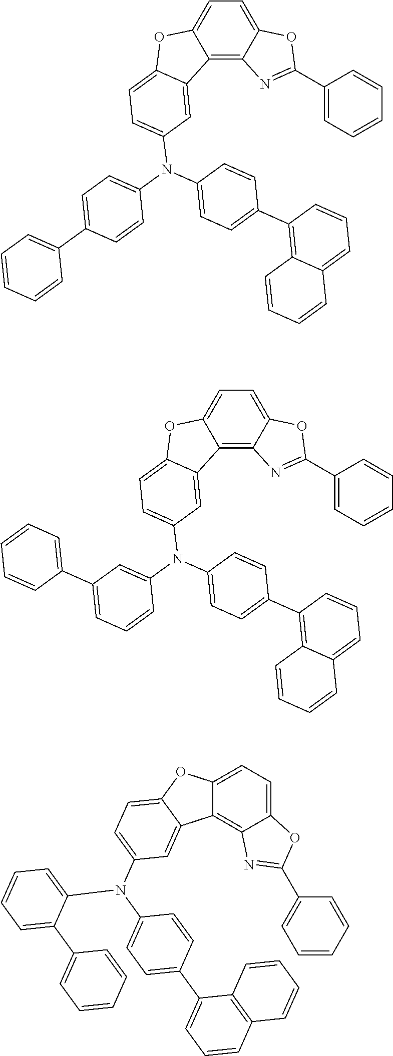 [SMILES: c1ccc(-c2ccc(N(c3ccc(-c4cccc5ccccc45)cc3)c3ccc4oc5ccc6oc(-c7ccccc7)nc6c5c4c3)cc2)cc1 ; c1ccc(-c2cccc(N(c3ccc(-c4cccc5ccccc45)cc3)c3ccc4oc5ccc6oc(-c7ccccc7)nc6c5c4c3)c2)cc1 ; c1ccc(-c2nc3c(ccc4oc5ccc(N(c6ccc(-c7cccc8ccccc78)cc6)c6ccccc6-c6ccccc6)cc5c43)o2)cc1]

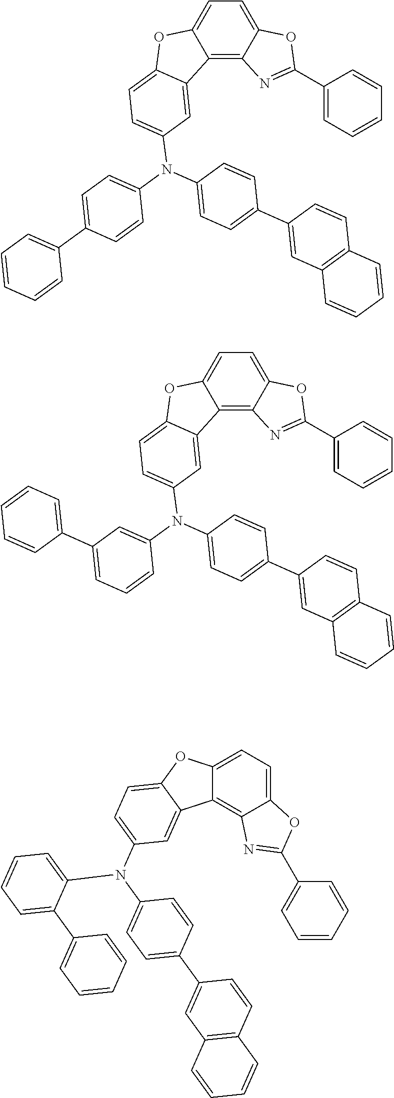 [SMILES: c1ccc(-c2ccc(N(c3ccc(-c4ccc5ccccc5c4)cc3)c3ccc4oc5ccc6oc(-c7ccccc7)nc6c5c4c3)cc2)cc1 ; c1ccc(-c2cccc(N(c3ccc(-c4ccc5ccccc5c4)cc3)c3ccc4oc5ccc6oc(-c7ccccc7)nc6c5c4c3)c2)cc1 ; c1ccc(-c2nc3c(ccc4oc5ccc(N(c6ccc(-c7ccc8ccccc8c7)cc6)c6ccccc6-c6ccccc6)cc5c43)o2)cc1]

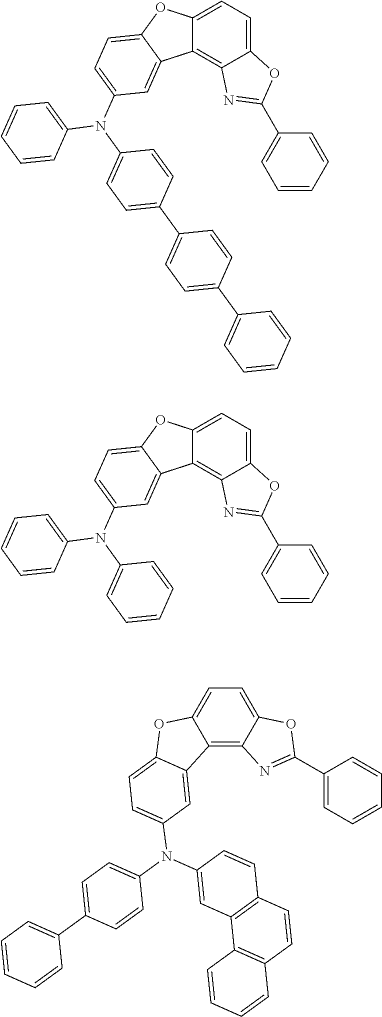 [SMILES: c1ccc(-c2ccc(-c3ccc(N(c4ccccc4)c4ccc5oc6ccc7oc(-c8ccccc8)nc7c6c5c4)cc3)cc2)cc1 ; c1ccc(-c2nc3c(ccc4oc5ccc(N(c6ccccc6)c6ccccc6)cc5c43)o2)cc1 ; c1ccc(-c2ccc(N(c3ccc4ccc5ccccc5c4c3)c3ccc4oc5ccc6oc(-c7ccccc7)nc6c5c4c3)cc2)cc1]

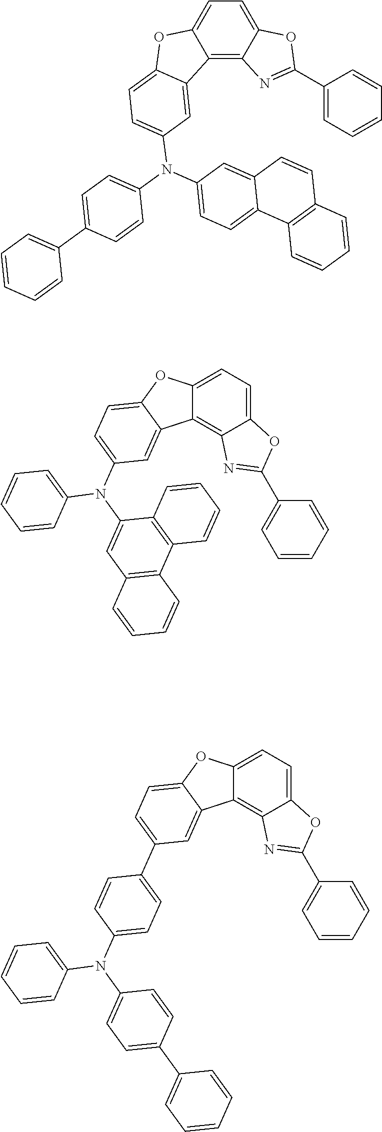 [SMILES: c1ccc(-c2ccc(N(c3ccc4c(ccc5ccccc54)c3)c3ccc4oc5ccc6oc(-c7ccccc7)nc6c5c4c3)cc2)cc1 ; c1ccc(-c2nc3c(ccc4oc5ccc(N(c6ccccc6)c6cc7ccccc7c7ccccc67)cc5c43)o2)cc1 ; c1ccc(-c2ccc(N(c3ccccc3)c3ccc(-c4ccc5oc6ccc7oc(-c8ccccc8)nc7c6c5c4)cc3)cc2)cc1]

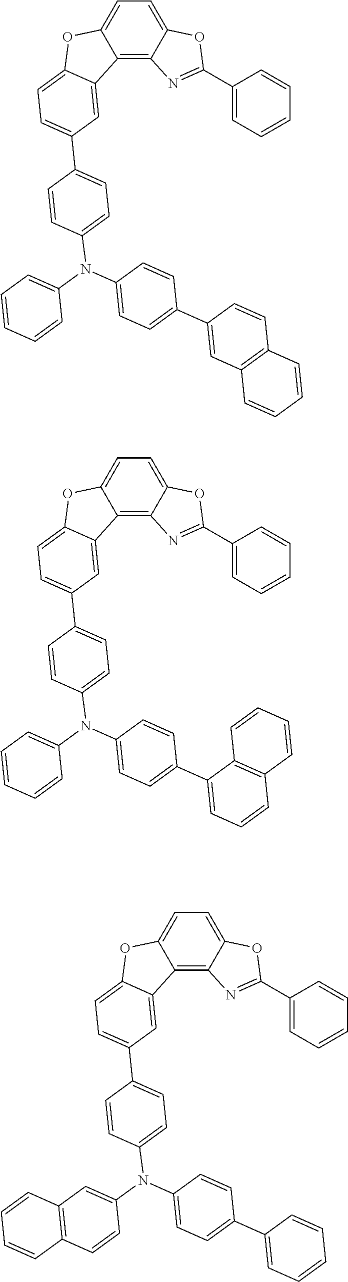 [SMILES: c1ccc(-c2nc3c(ccc4oc5ccc(-c6ccc(N(c7ccccc7)c7ccc(-c8ccc9ccccc9c8)cc7)cc6)cc5c43)o2)cc1 ; c1ccc(-c2nc3c(ccc4oc5ccc(-c6ccc(N(c7ccccc7)c7ccc(-c8cccc9ccccc89)cc7)cc6)cc5c43)o2)cc1 ; c1ccc(-c2ccc(N(c3ccc(-c4ccc5oc6ccc7oc(-c8ccccc8)nc7c6c5c4)cc3)c3ccc4ccccc4c3)cc2)cc1]

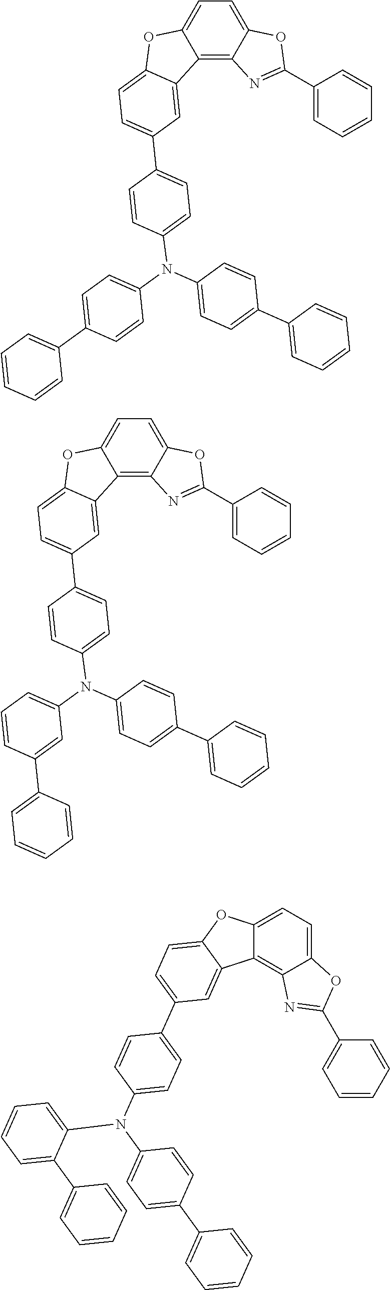 [SMILES: c1ccc(-c2ccc(N(c3ccc(-c4ccccc4)cc3)c3ccc(-c4ccc5oc6ccc7oc(-c8ccccc8)nc7c6c5c4)cc3)cc2)cc1 ; c1ccc(-c2ccc(N(c3ccc(-c4ccc5oc6ccc7oc(-c8ccccc8)nc7c6c5c4)cc3)c3cccc(-c4ccccc4)c3)cc2)cc1 ; c1ccc(-c2ccc(N(c3ccc(-c4ccc5oc6ccc7oc(-c8ccccc8)nc7c6c5c4)cc3)c3ccccc3-c3ccccc3)cc2)cc1]

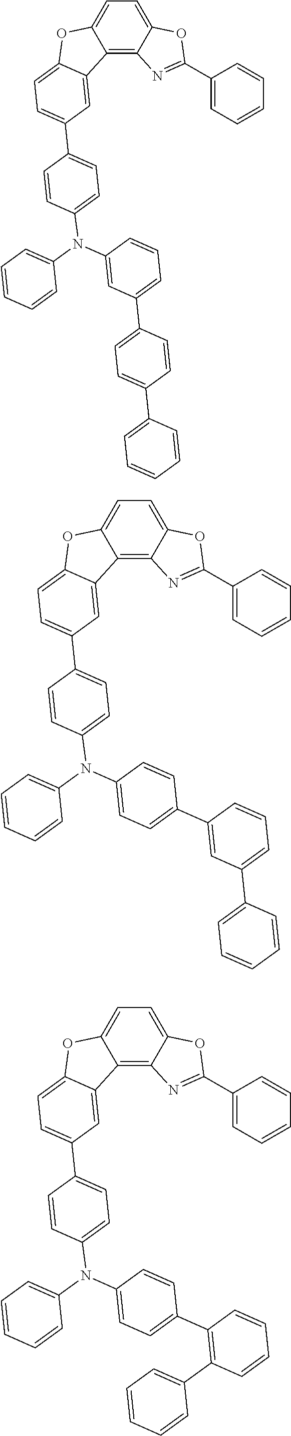

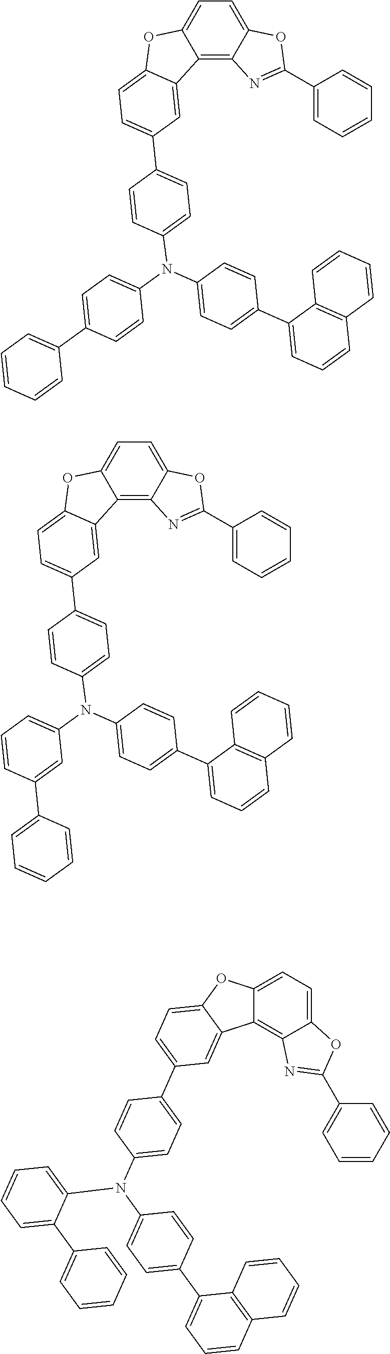 [SMILES: c1ccc(-c2ccc(N(c3ccc(-c4ccc5oc6ccc7oc(-c8ccccc8)nc7c6c5c4)cc3)c3ccc(-c4cccc5ccccc45)cc3)cc2)cc1 ; c1ccc(-c2cccc(N(c3ccc(-c4ccc5oc6ccc7oc(-c8ccccc8)nc7c6c5c4)cc3)c3ccc(-c4cccc5ccccc45)cc3)c2)cc1 ; c1ccc(-c2nc3c(ccc4oc5ccc(-c6ccc(N(c7ccc(-c8cccc9ccccc89)cc7)c7ccccc7-c7ccccc7)cc6)cc5c43)o2)cc1]

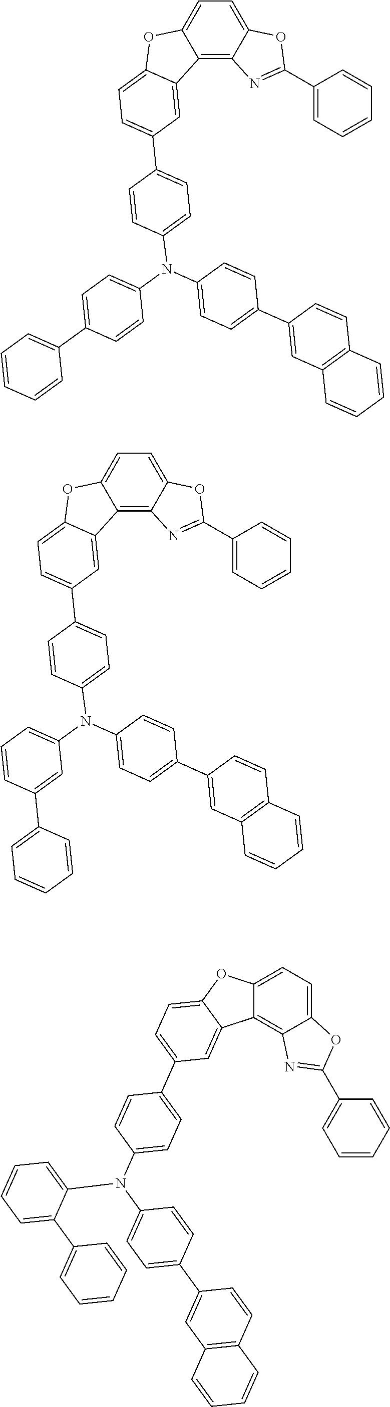 [SMILES: c1ccc(-c2ccc(N(c3ccc(-c4ccc5ccccc5c4)cc3)c3ccc(-c4ccc5oc6ccc7oc(-c8ccccc8)nc7c6c5c4)cc3)cc2)cc1 ; c1ccc(-c2cccc(N(c3ccc(-c4ccc5ccccc5c4)cc3)c3ccc(-c4ccc5oc6ccc7oc(-c8ccccc8)nc7c6c5c4)cc3)c2)cc1 ; c1ccc(-c2nc3c(ccc4oc5ccc(-c6ccc(N(c7ccc(-c8ccc9ccccc9c8)cc7)c7ccccc7-c7ccccc7)cc6)cc5c43)o2)cc1]

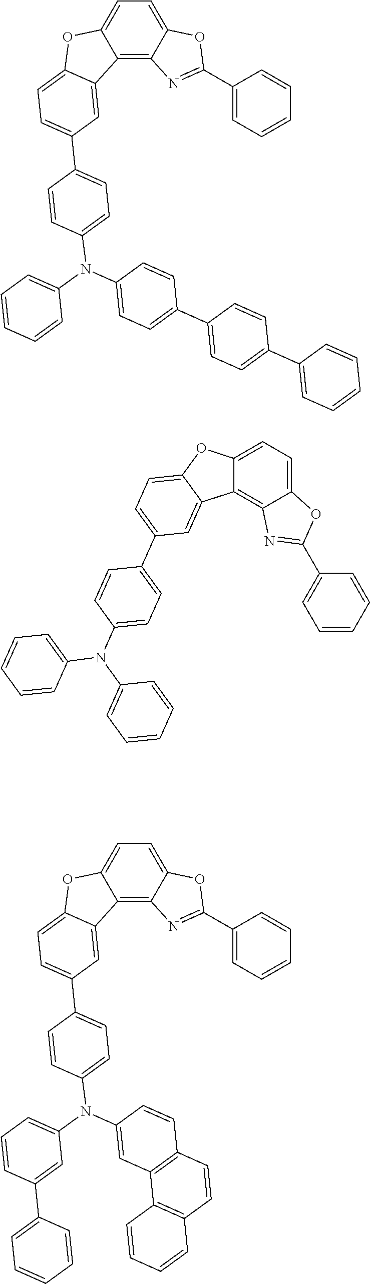 [SMILES: c1ccc(-c2ccc(-c3ccc(N(c4ccccc4)c4ccc(-c5ccc6oc7ccc8oc(-c9ccccc9)nc8c7c6c5)cc4)cc3)cc2)cc1 ; c1ccc(-c2nc3c(ccc4oc5ccc(-c6ccc(N(c7ccccc7)c7ccccc7)cc6)cc5c43)o2)cc1 ; c1ccc(-c2cccc(N(c3ccc(-c4ccc5oc6ccc7oc(-c8ccccc8)nc7c6c5c4)cc3)c3ccc4ccc5ccccc5c4c3)c2)cc1]

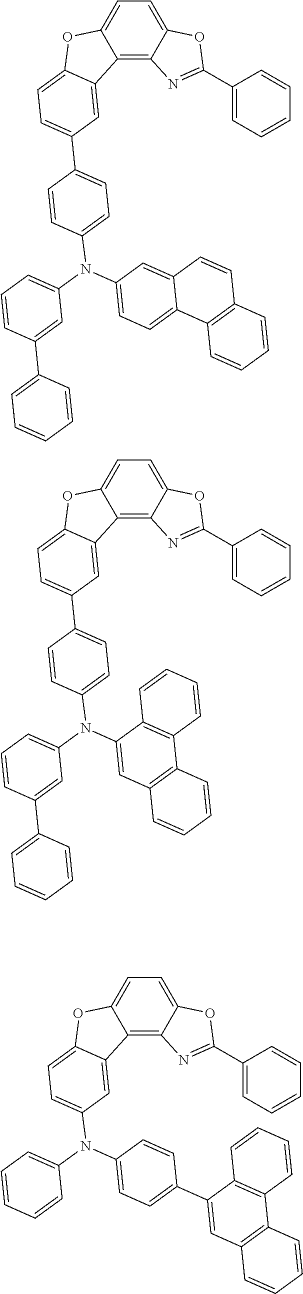 [SMILES: c1ccc(-c2cccc(N(c3ccc(-c4ccc5oc6ccc7oc(-c8ccccc8)nc7c6c5c4)cc3)c3ccc4c(ccc5ccccc54)c3)c2)cc1 ; c1ccc(-c2cccc(N(c3ccc(-c4ccc5oc6ccc7oc(-c8ccccc8)nc7c6c5c4)cc3)c3cc4ccccc4c4ccccc34)c2)cc1 ; c1ccc(-c2nc3c(ccc4oc5ccc(N(c6ccccc6)c6ccc(-c7cc8ccccc8c8ccccc78)cc6)cc5c43)o2)cc1]

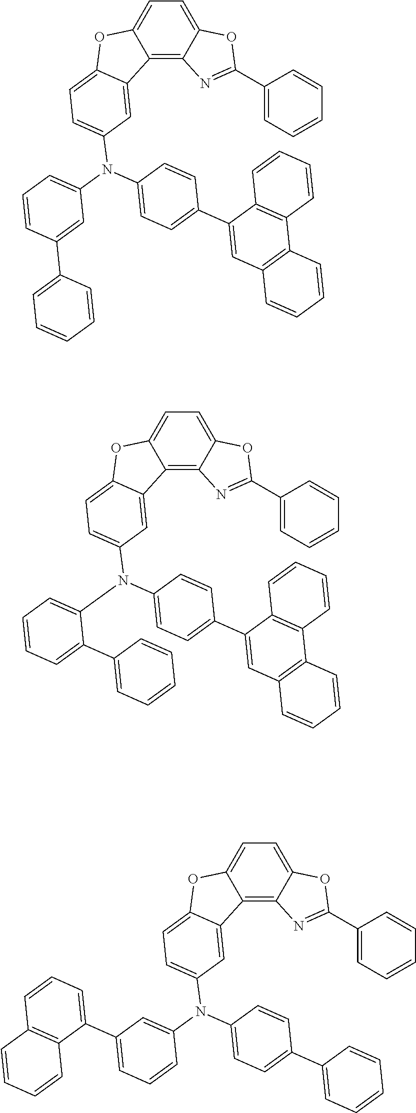 [SMILES: c1ccc(-c2cccc(N(c3ccc(-c4cc5ccccc5c5ccccc45)cc3)c3ccc4oc5ccc6oc(-c7ccccc7)nc6c5c4c3)c2)cc1 ; c1ccc(-c2nc3c(ccc4oc5ccc(N(c6ccc(-c7cc8ccccc8c8ccccc78)cc6)c6ccccc6-c6ccccc6)cc5c43)o2)cc1 ; c1ccc(-c2ccc(N(c3cccc(-c4cccc5ccccc45)c3)c3ccc4oc5ccc6oc(-c7ccccc7)nc6c5c4c3)cc2)cc1]

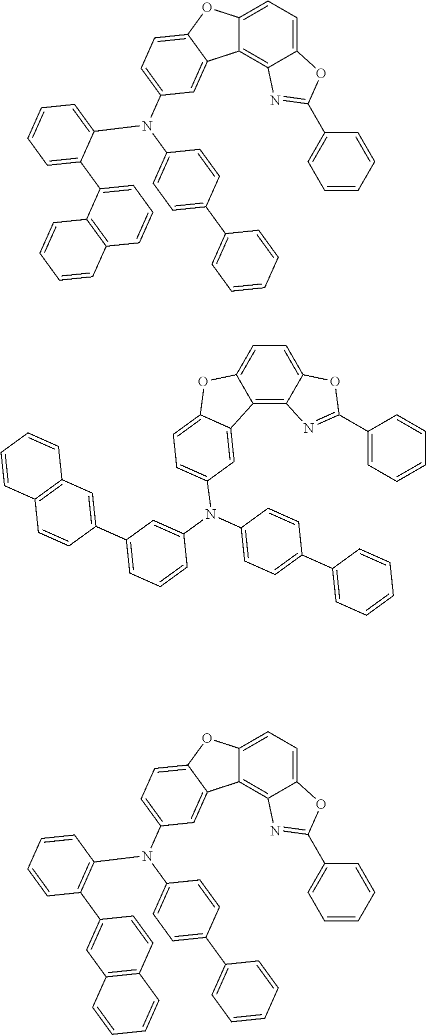 [SMILES: c1ccc(-c2ccc(N(c3ccc4oc5ccc6oc(-c7ccccc7)nc6c5c4c3)c3ccccc3-c3cccc4ccccc34)cc2)cc1 ; c1ccc(-c2ccc(N(c3cccc(-c4ccc5ccccc5c4)c3)c3ccc4oc5ccc6oc(-c7ccccc7)nc6c5c4c3)cc2)cc1 ; c1ccc(-c2ccc(N(c3ccc4oc5ccc6oc(-c7ccccc7)nc6c5c4c3)c3ccccc3-c3ccc4ccccc4c3)cc2)cc1]

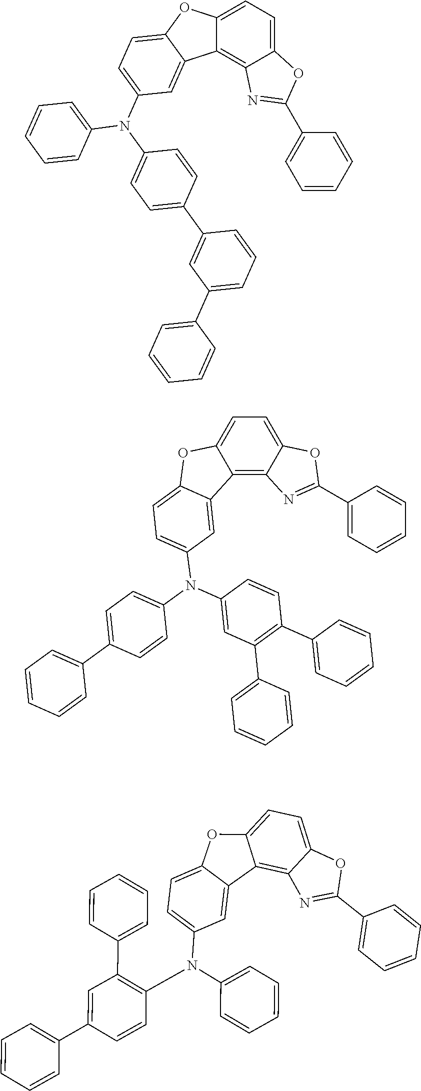 [SMILES: c1ccc(-c2cccc(-c3ccc(N(c4ccccc4)c4ccc5oc6ccc7oc(-c8ccccc8)nc7c6c5c4)cc3)c2)cc1 ; c1ccc(-c2ccc(N(c3ccc(-c4ccccc4)c(-c4ccccc4)c3)c3ccc4oc5ccc6oc(-c7ccccc7)nc6c5c4c3)cc2)cc1 ; c1ccc(-c2ccc(N(c3ccccc3)c3ccc4oc5ccc6oc(-c7ccccc7)nc6c5c4c3)c(-c3ccccc3)c2)cc1]

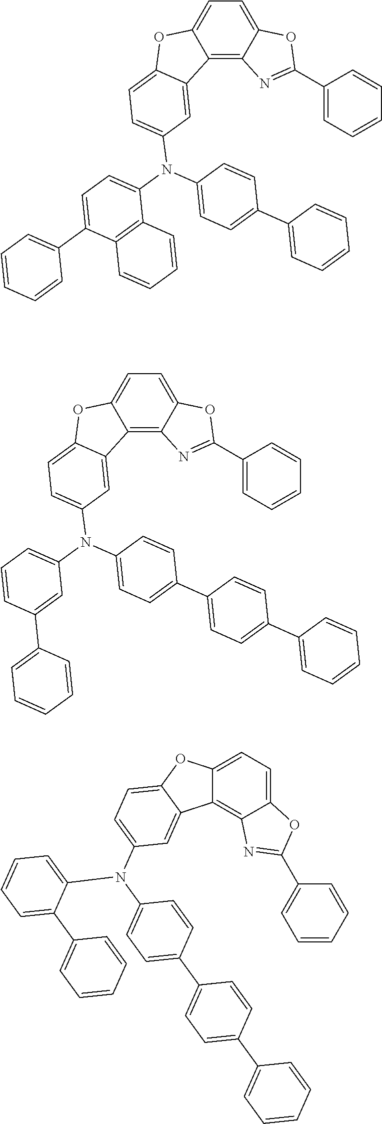 [SMILES: c1ccc(-c2ccc(N(c3ccc4oc5ccc6oc(-c7ccccc7)nc6c5c4c3)c3ccc(-c4ccccc4)c4ccccc34)cc2)cc1 ; c1ccc(-c2ccc(-c3ccc(N(c4cccc(-c5ccccc5)c4)c4ccc5oc6ccc7oc(-c8ccccc8)nc7c6c5c4)cc3)cc2)cc1 ; c1ccc(-c2ccc(-c3ccc(N(c4ccc5oc6ccc7oc(-c8ccccc8)nc7c6c5c4)c4ccccc4-c4ccccc4)cc3)cc2)cc1]

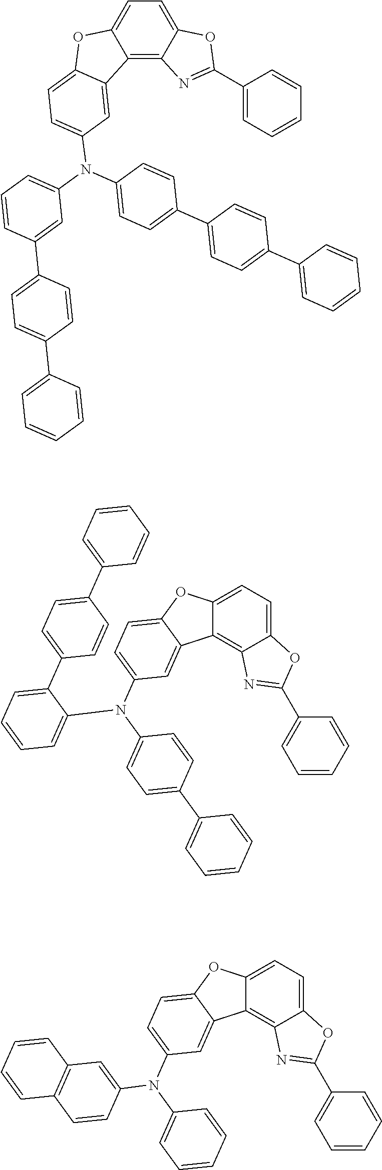 [SMILES: c1ccc(-c2ccc(-c3ccc(N(c4cccc(-c5ccc(-c6ccccc6)cc5)c4)c4ccc5oc6ccc7oc(-c8ccccc8)nc7c6c5c4)cc3)cc2)cc1 ; c1ccc(-c2ccc(-c3ccccc3N(c3ccc(-c4ccccc4)cc3)c3ccc4oc5ccc6oc(-c7ccccc7)nc6c5c4c3)cc2)cc1 ; c1ccc(-c2nc3c(ccc4oc5ccc(N(c6ccccc6)c6ccc7ccccc7c6)cc5c43)o2)cc1]

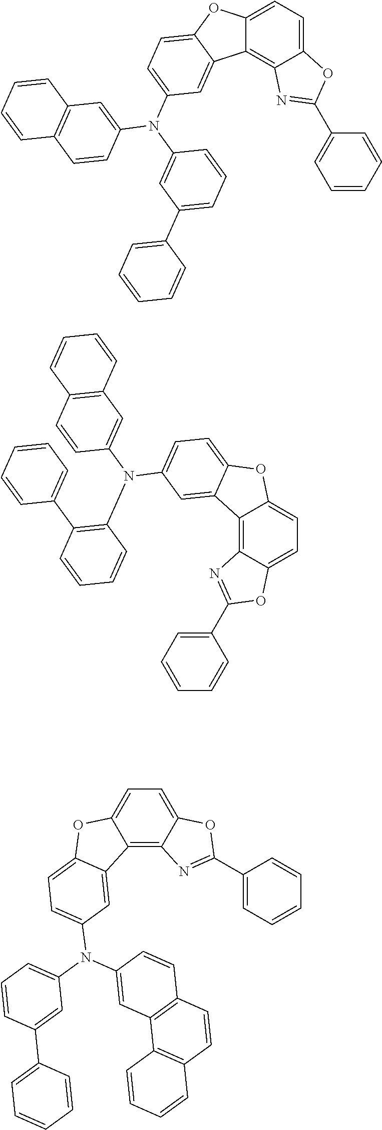 [SMILES: c1ccc(-c2cccc(N(c3ccc4ccccc4c3)c3ccc4oc5ccc6oc(-c7ccccc7)nc6c5c4c3)c2)cc1 ; c1ccc(-c2nc3c(ccc4oc5ccc(N(c6ccc7ccccc7c6)c6ccccc6-c6ccccc6)cc5c43)o2)cc1 ; c1ccc(-c2cccc(N(c3ccc4ccc5ccccc5c4c3)c3ccc4oc5ccc6oc(-c7ccccc7)nc6c5c4c3)c2)cc1]

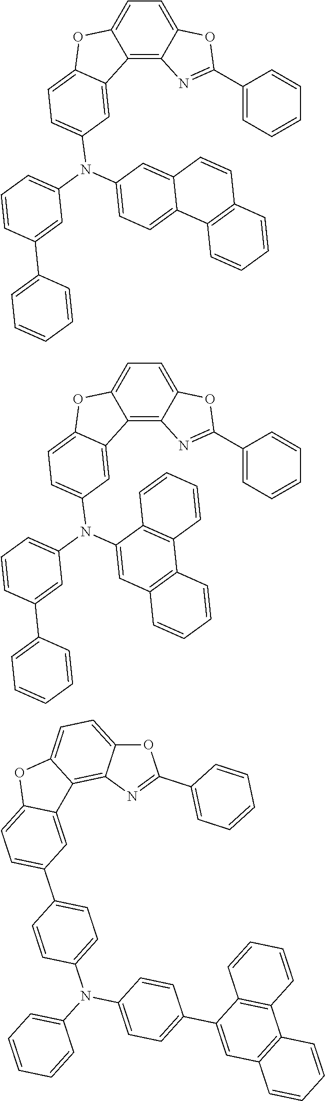

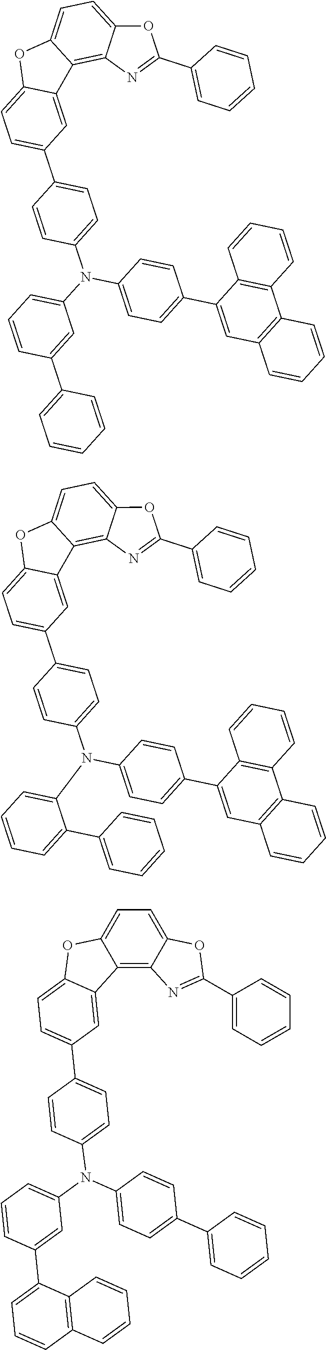

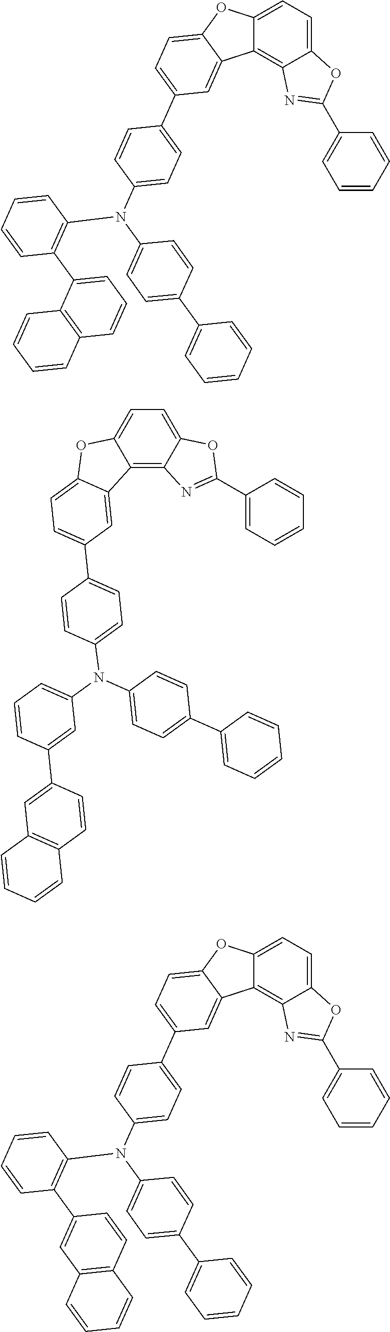 [SMILES: c1ccc(-c2ccc(N(c3ccc(-c4ccc5oc6ccc7oc(-c8ccccc8)nc7c6c5c4)cc3)c3ccccc3-c3cccc4ccccc34)cc2)cc1 ; c1ccc(-c2ccc(N(c3ccc(-c4ccc5oc6ccc7oc(-c8ccccc8)nc7c6c5c4)cc3)c3cccc(-c4ccc5ccccc5c4)c3)cc2)cc1 ; c1ccc(-c2ccc(N(c3ccc(-c4ccc5oc6ccc7oc(-c8ccccc8)nc7c6c5c4)cc3)c3ccccc3-c3ccc4ccccc4c3)cc2)cc1]

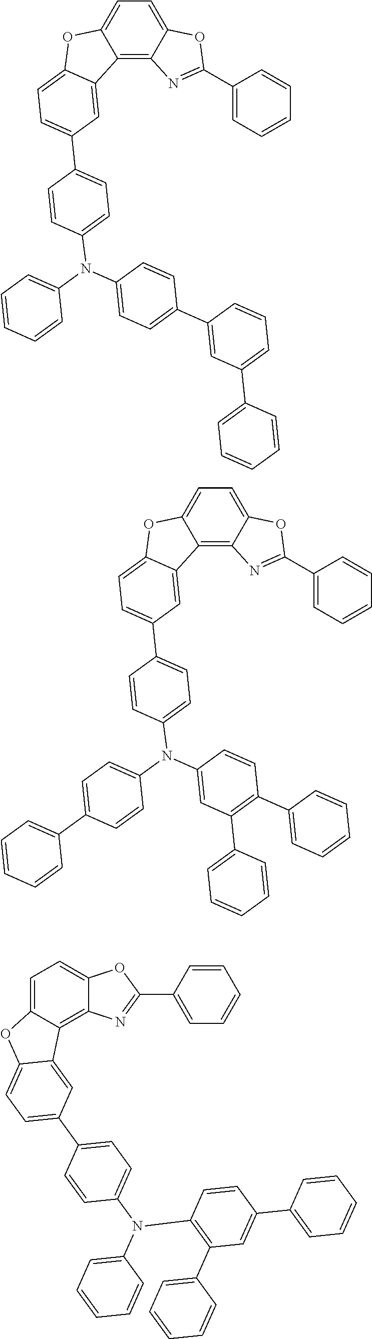

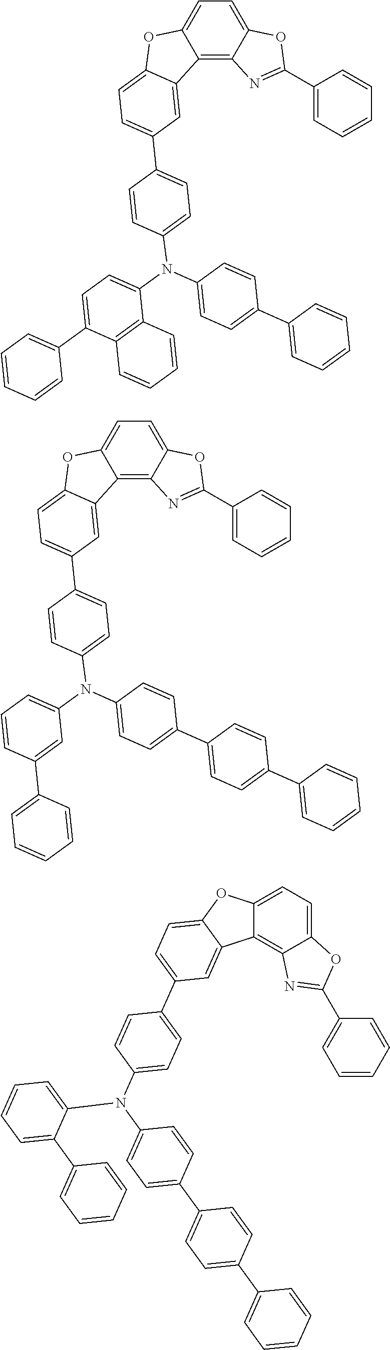

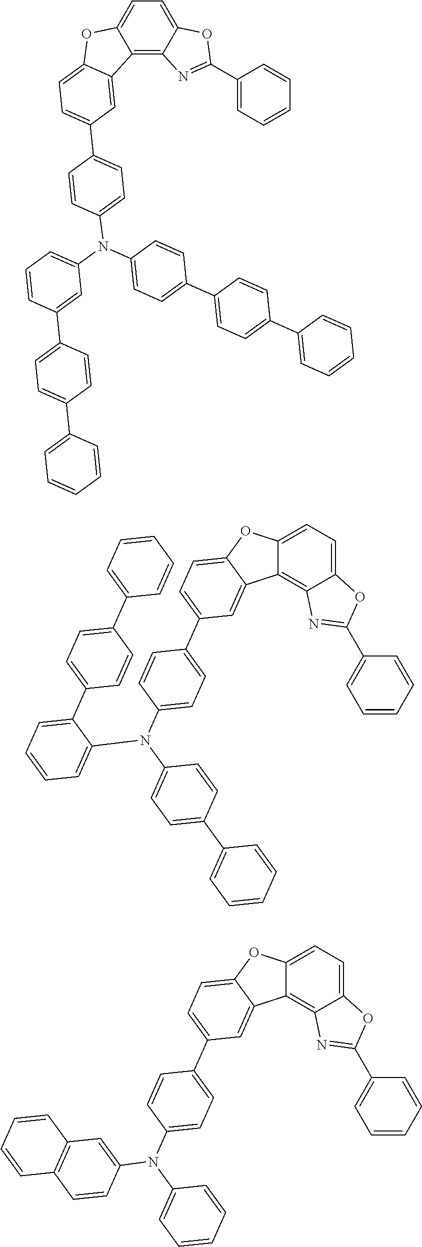 [SMILES: c1ccc(-c2ccc(-c3ccc(N(c4ccc(-c5ccc6oc7ccc8oc(-c9ccccc9)nc8c7c6c5)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4ccc5oc6ccc7oc(-c8ccccc8)nc7c6c5c4)cc3)cc2)cc1 ; c1ccc(-c2nc3c(ccc4oc5ccc(-c6ccc(N(c7ccccc7)c7ccc8ccccc8c7)cc6)cc5c43)o2)cc1]

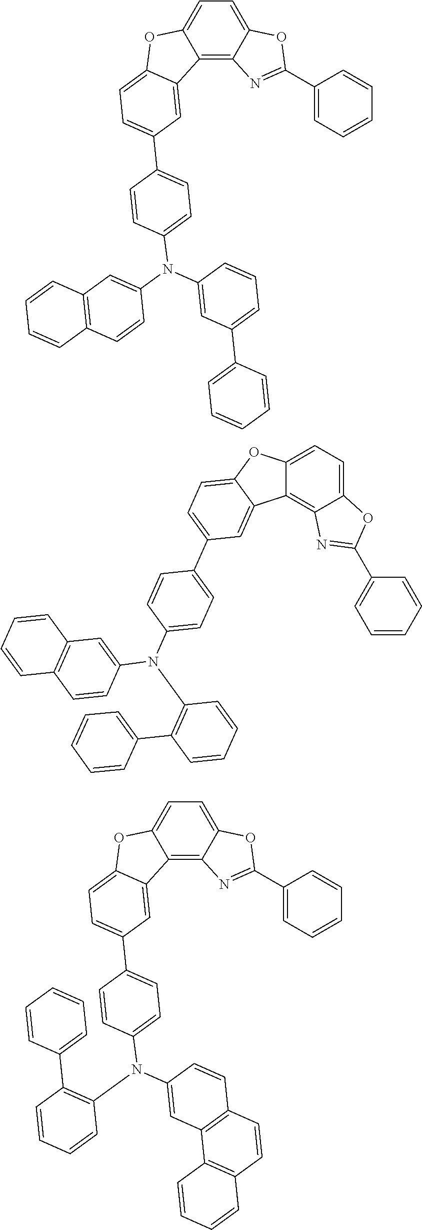

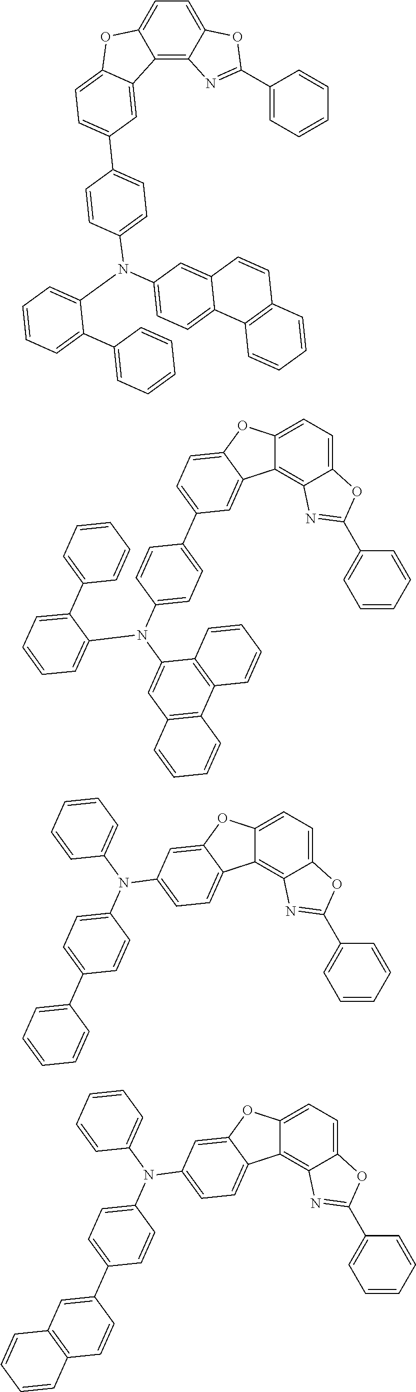

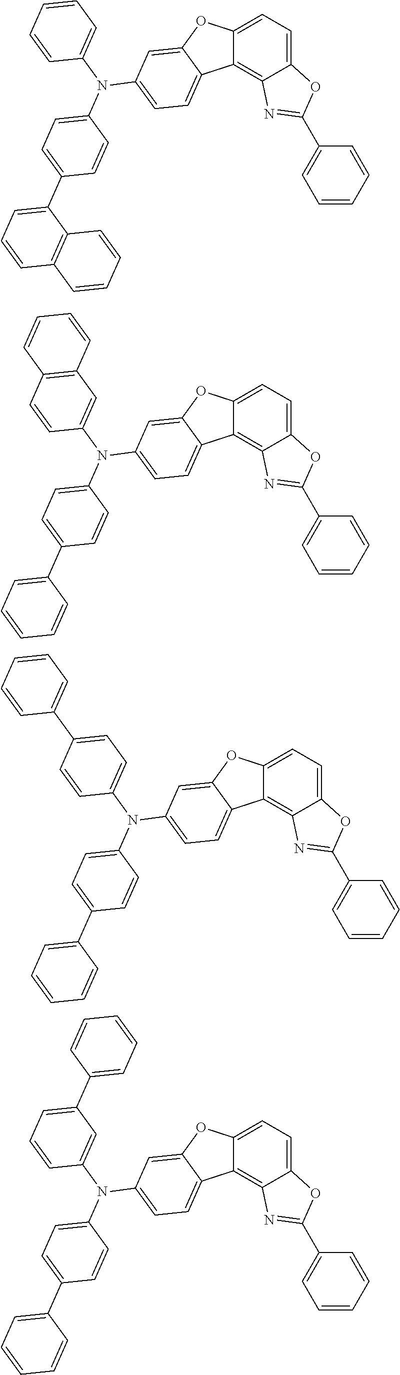

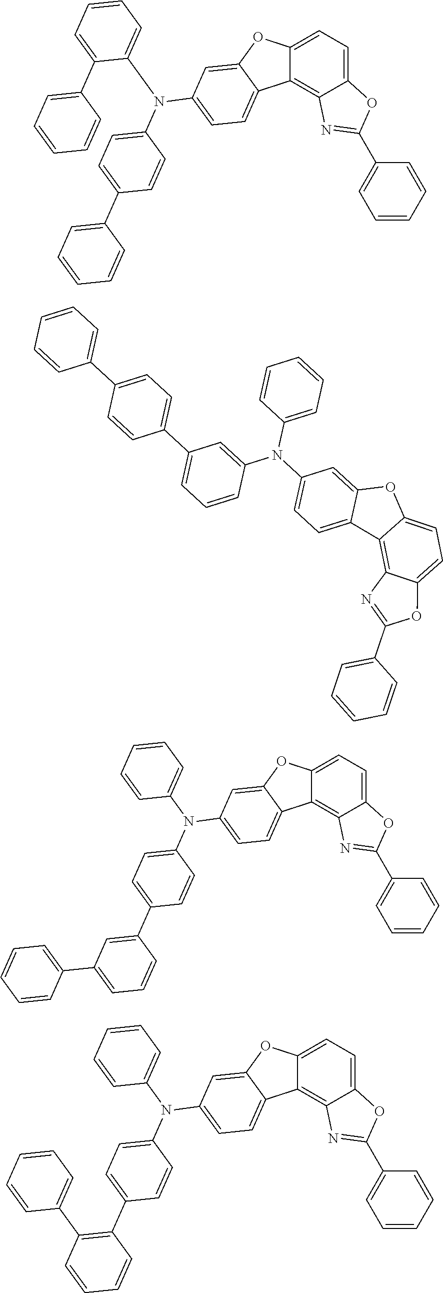

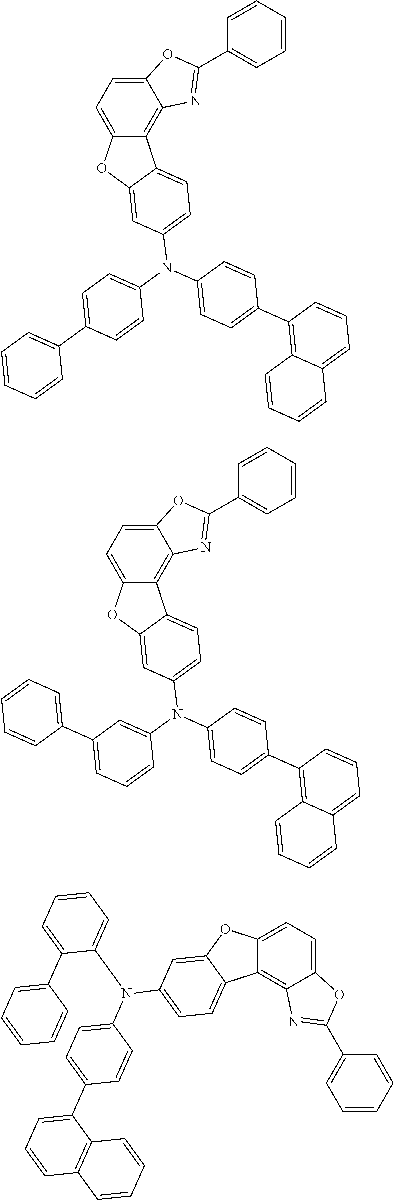

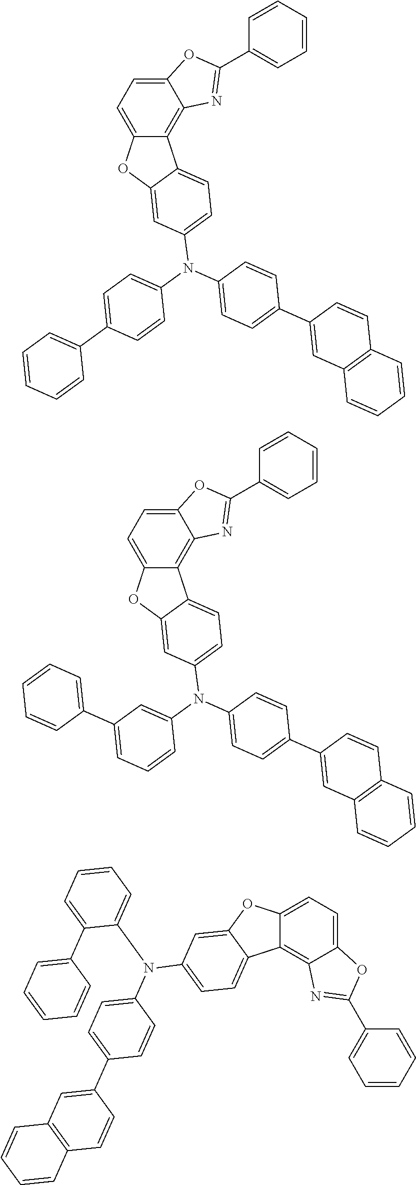 [SMILES: c1ccc(-c2ccc(N(c3ccc(-c4ccc5ccccc5c4)cc3)c3ccc4c(c3)oc3ccc5oc(-c6ccccc6)nc5c34)cc2)cc1 ; c1ccc(-c2cccc(N(c3ccc(-c4ccc5ccccc5c4)cc3)c3ccc4c(c3)oc3ccc5oc(-c6ccccc6)nc5c34)c2)cc1 ; c1ccc(-c2nc3c(ccc4oc5cc(N(c6ccc(-c7ccc8ccccc8c7)cc6)c6ccccc6-c6ccccc6)ccc5c43)o2)cc1]

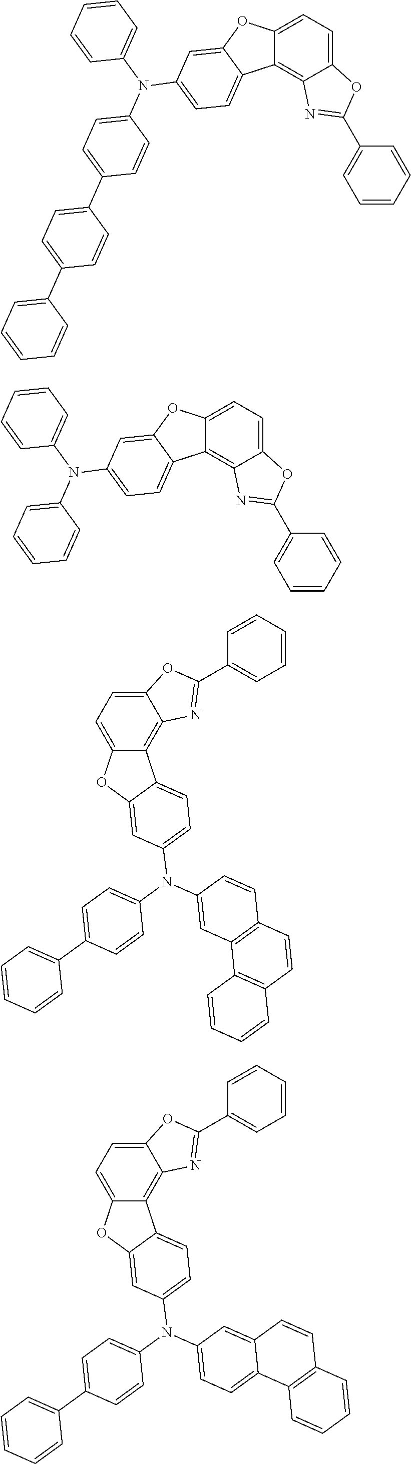

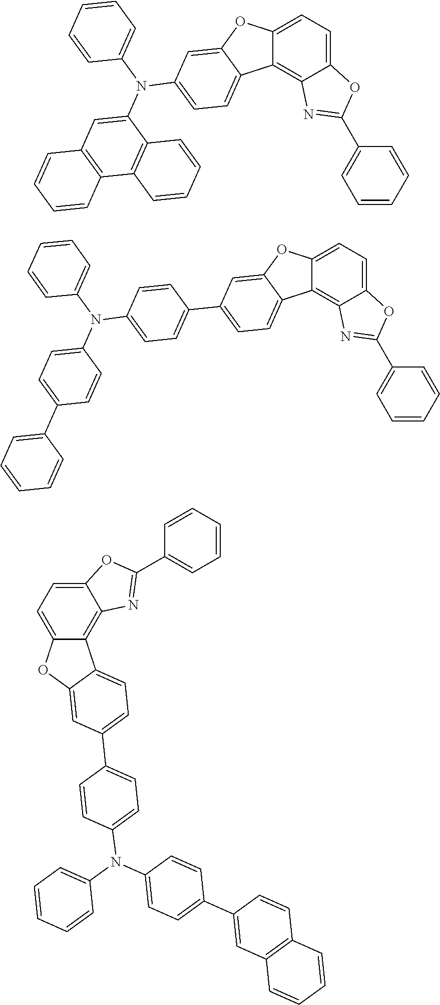

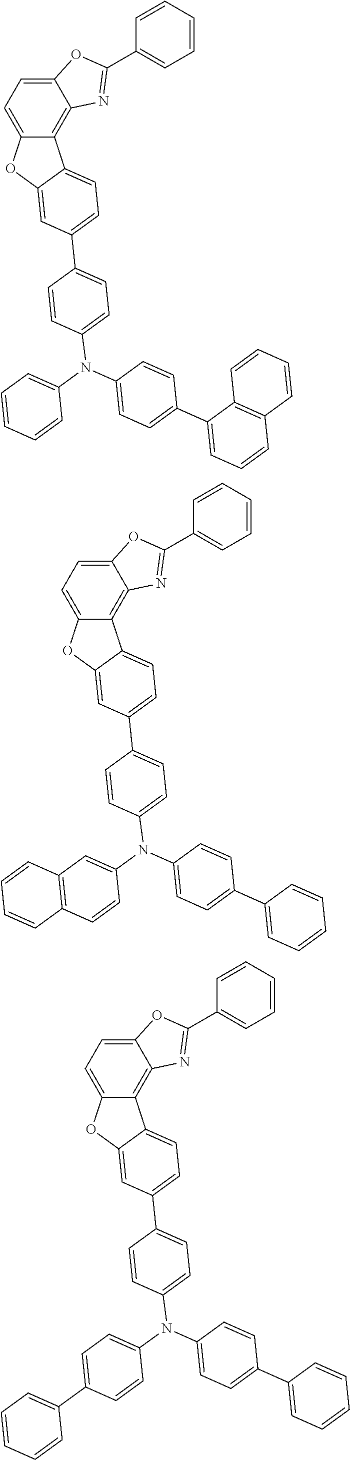

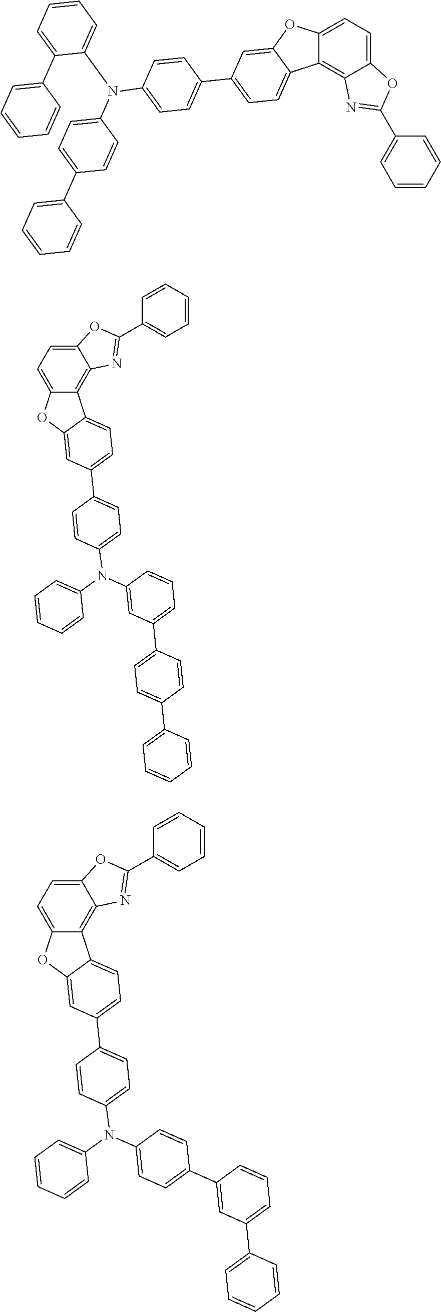

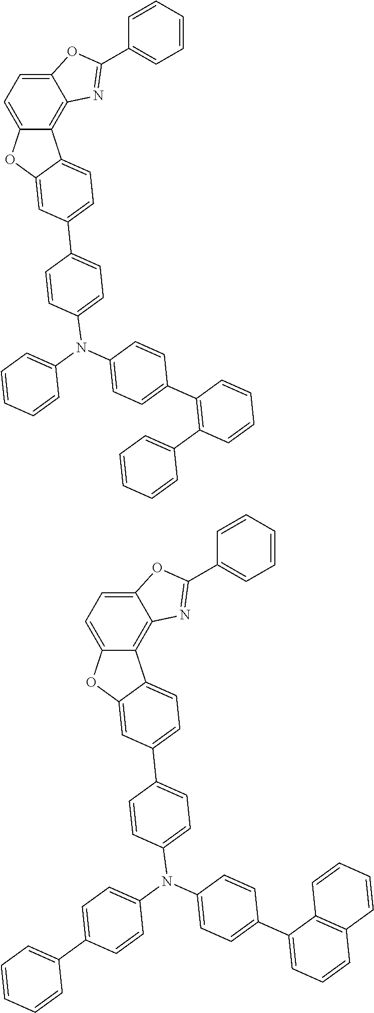 [SMILES: c1ccc(-c2nc3c(ccc4oc5cc(-c6ccc(N(c7ccccc7)c7ccc(-c8ccccc8-c8ccccc8)cc7)cc6)ccc5c43)o2)cc1 ; c1ccc(-c2ccc(N(c3ccc(-c4ccc5c(c4)oc4ccc6oc(-c7ccccc7)nc6c45)cc3)c3ccc(-c4cccc5ccccc45)cc3)cc2)cc1]

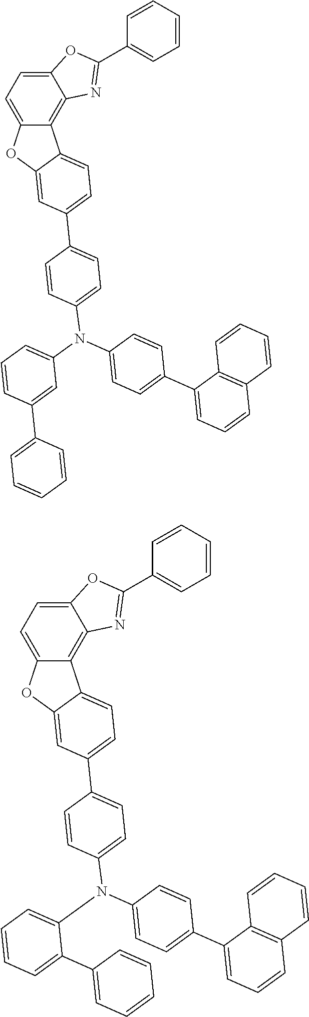 [SMILES: c1ccc(-c2cccc(N(c3ccc(-c4ccc5c(c4)oc4ccc6oc(-c7ccccc7)nc6c45)cc3)c3ccc(-c4cccc5ccccc45)cc3)c2)cc1 ; c1ccc(-c2nc3c(ccc4oc5cc(-c6ccc(N(c7ccc(-c8cccc9ccccc89)cc7)c7ccccc7-c7ccccc7)cc6)ccc5c43)o2)cc1]

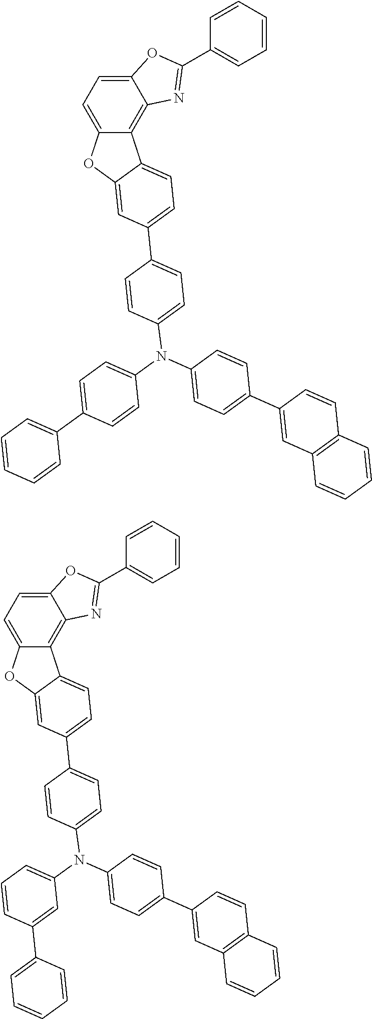 [SMILES: c1ccc(-c2ccc(N(c3ccc(-c4ccc5ccccc5c4)cc3)c3ccc(-c4ccc5c(c4)oc4ccc6oc(-c7ccccc7)nc6c45)cc3)cc2)cc1 ; c1ccc(-c2cccc(N(c3ccc(-c4ccc5ccccc5c4)cc3)c3ccc(-c4ccc5c(c4)oc4ccc6oc(-c7ccccc7)nc6c45)cc3)c2)cc1]

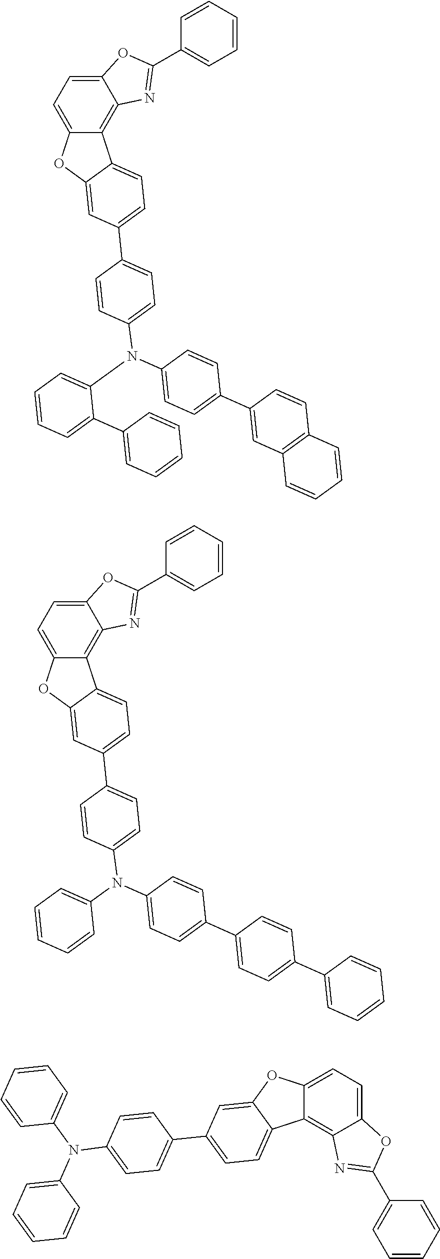 [SMILES: c1ccc(-c2nc3c(ccc4oc5cc(-c6ccc(N(c7ccc(-c8ccc9ccccc9c8)cc7)c7ccccc7-c7ccccc7)cc6)ccc5c43)o2)cc1 ; c1ccc(-c2ccc(-c3ccc(N(c4ccccc4)c4ccc(-c5ccc6c(c5)oc5ccc7oc(-c8ccccc8)nc7c56)cc4)cc3)cc2)cc1 ; c1ccc(-c2nc3c(ccc4oc5cc(-c6ccc(N(c7ccccc7)c7ccccc7)cc6)ccc5c43)o2)cc1]

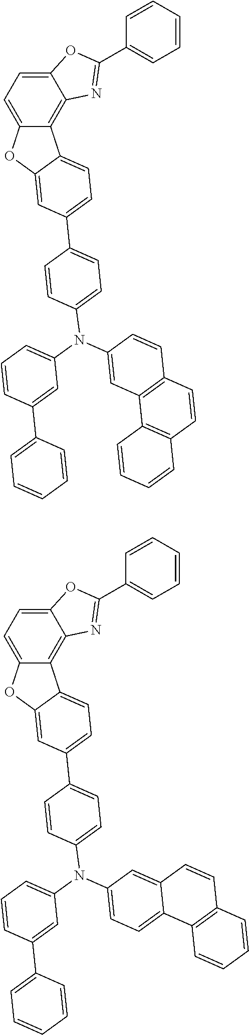 [SMILES: c1ccc(-c2cccc(N(c3ccc(-c4ccc5c(c4)oc4ccc6oc(-c7ccccc7)nc6c45)cc3)c3ccc4ccc5ccccc5c4c3)c2)cc1 ; c1ccc(-c2cccc(N(c3ccc(-c4ccc5c(c4)oc4ccc6oc(-c7ccccc7)nc6c45)cc3)c3ccc4c(ccc5ccccc54)c3)c2)cc1]

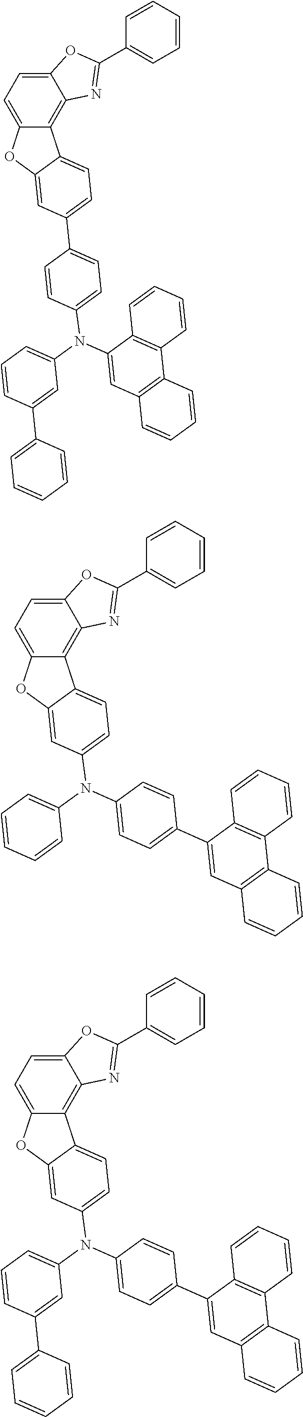 [SMILES: c1ccc(-c2cccc(N(c3ccc(-c4ccc5c(c4)oc4ccc6oc(-c7ccccc7)nc6c45)cc3)c3cc4ccccc4c4ccccc34)c2)cc1 ; c1ccc(-c2nc3c(ccc4oc5cc(N(c6ccccc6)c6ccc(-c7cc8ccccc8c8ccccc78)cc6)ccc5c43)o2)cc1 ; c1ccc(-c2cccc(N(c3ccc(-c4cc5ccccc5c5ccccc45)cc3)c3ccc4c(c3)oc3ccc5oc(-c6ccccc6)nc5c34)c2)cc1]

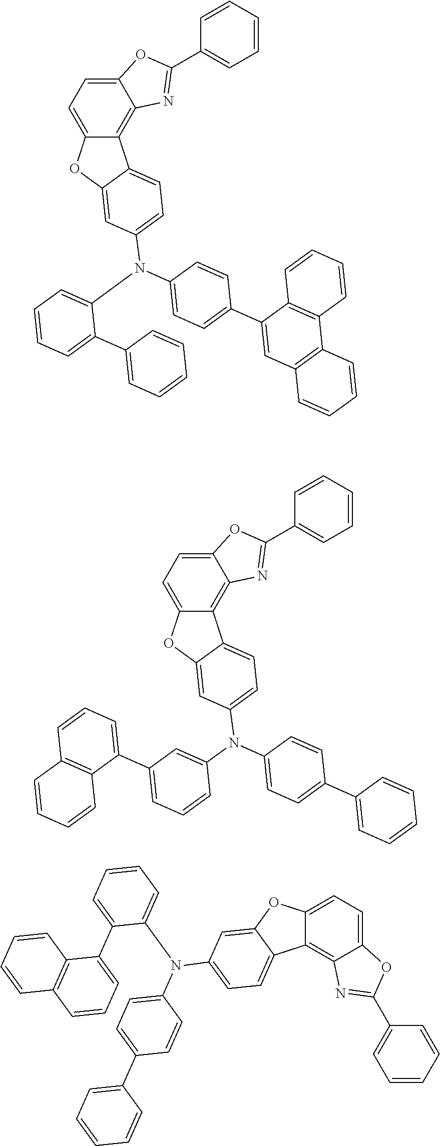 [SMILES: c1ccc(-c2nc3c(ccc4oc5cc(N(c6ccc(-c7cc8ccccc8c8ccccc78)cc6)c6ccccc6-c6ccccc6)ccc5c43)o2)cc1 ; c1ccc(-c2ccc(N(c3cccc(-c4cccc5ccccc45)c3)c3ccc4c(c3)oc3ccc5oc(-c6ccccc6)nc5c34)cc2)cc1 ; c1ccc(-c2ccc(N(c3ccc4c(c3)oc3ccc5oc(-c6ccccc6)nc5c34)c3ccccc3-c3cccc4ccccc34)cc2)cc1]

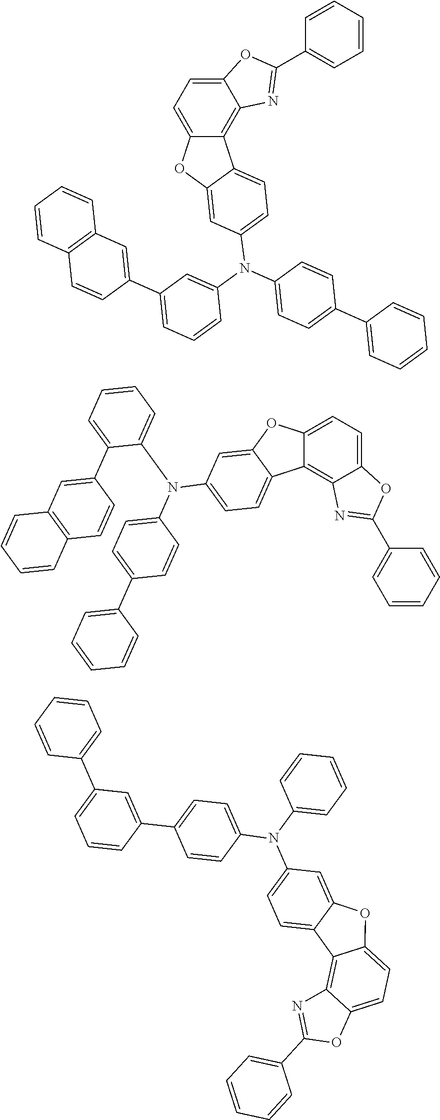

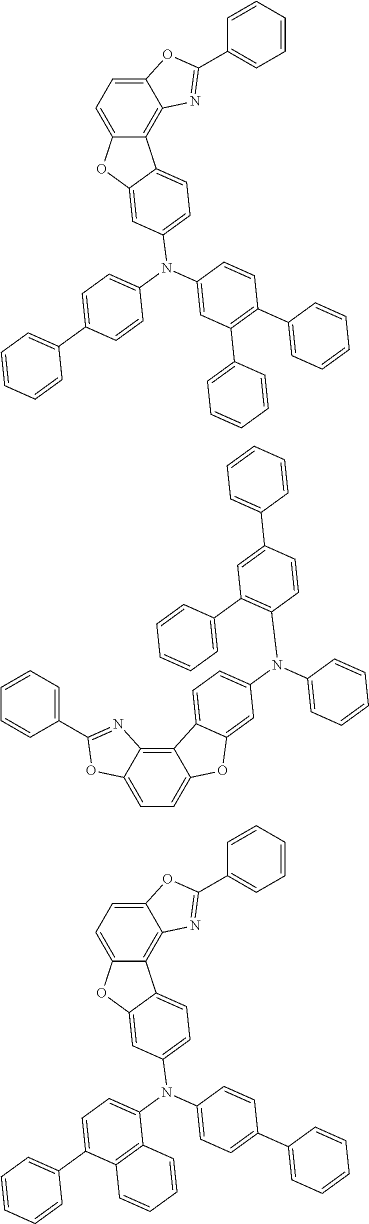

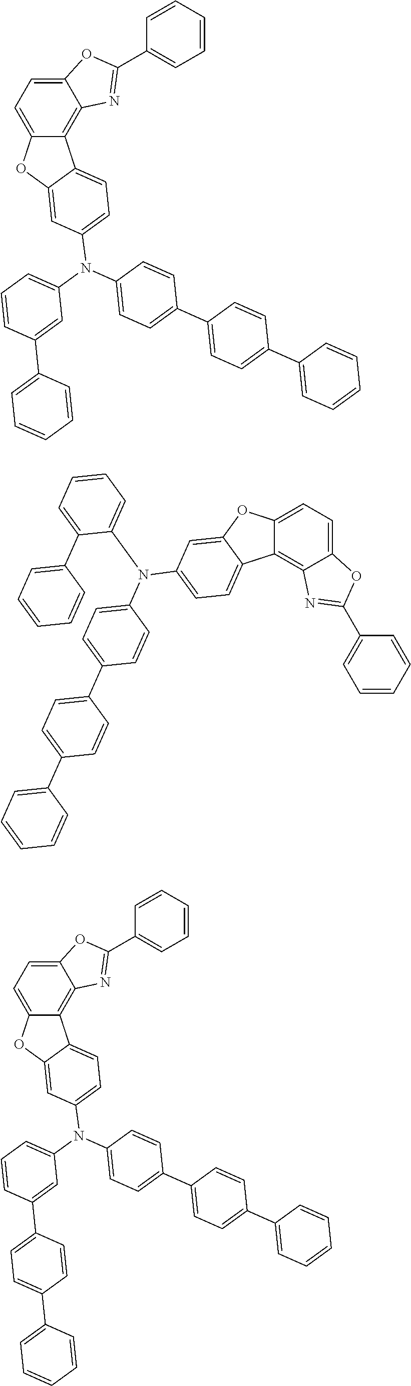

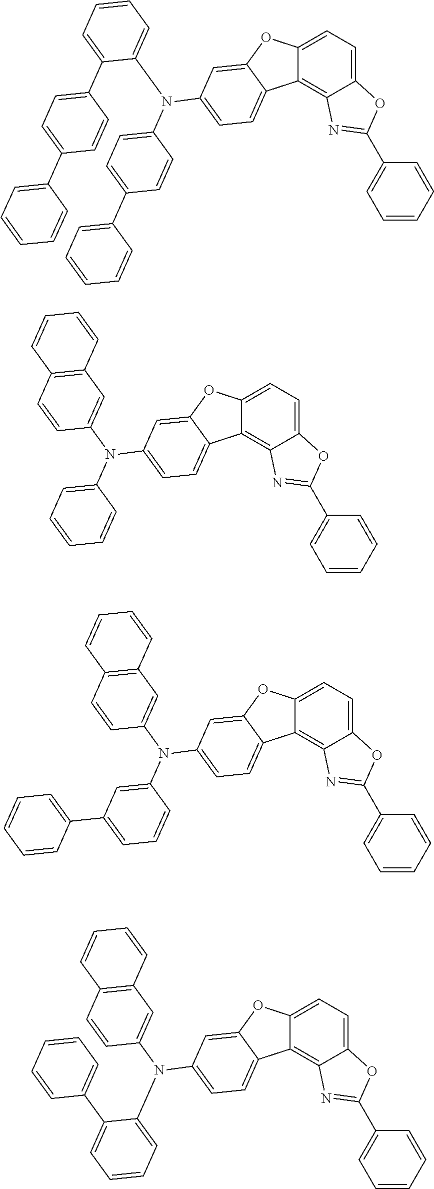

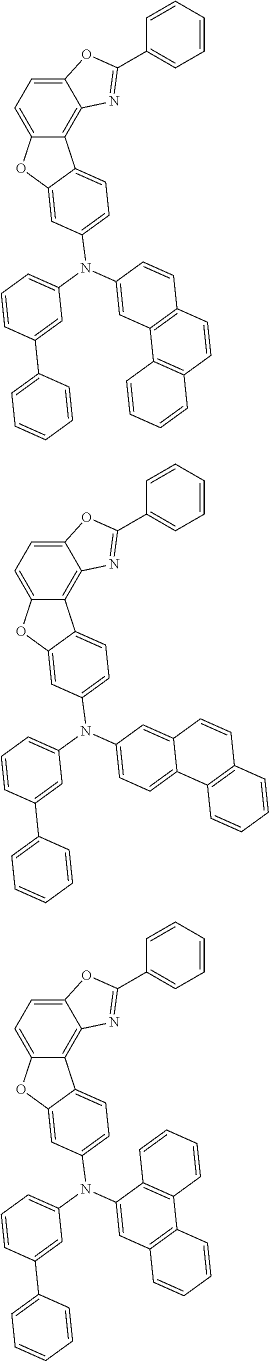

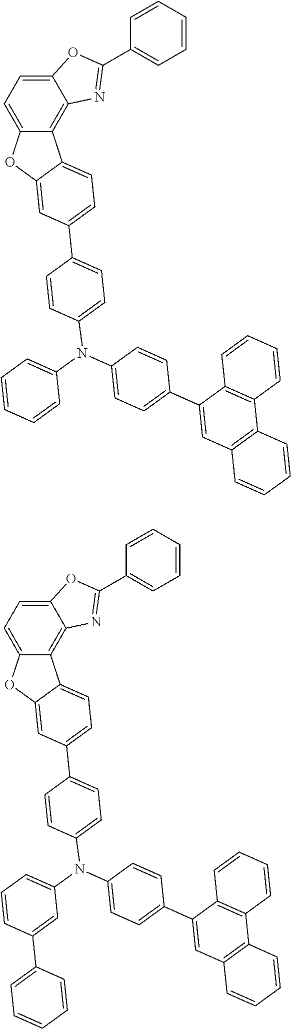 [SMILES: c1ccc(-c2nc3c(ccc4oc5cc(-c6ccc(N(c7ccccc7)c7ccc(-c8cc9ccccc9c9ccccc89)cc7)cc6)ccc5c43)o2)cc1 ; c1ccc(-c2cccc(N(c3ccc(-c4ccc5c(c4)oc4ccc6oc(-c7ccccc7)nc6c45)cc3)c3ccc(-c4cc5ccccc5c5ccccc45)cc3)c2)cc1]

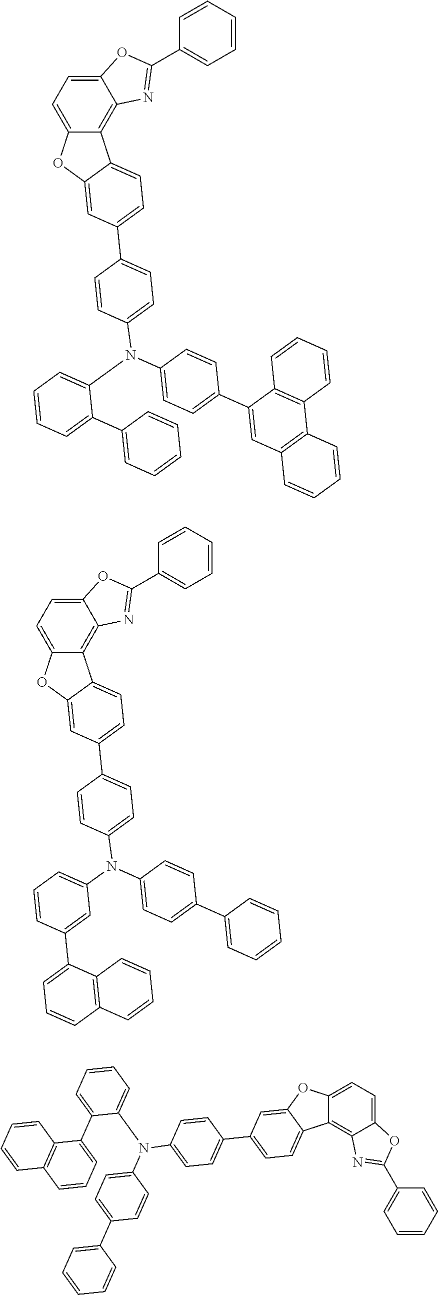 [SMILES: c1ccc(-c2nc3c(ccc4oc5cc(-c6ccc(N(c7ccc(-c8cc9ccccc9c9ccccc89)cc7)c7ccccc7-c7ccccc7)cc6)ccc5c43)o2)cc1 ; c1ccc(-c2ccc(N(c3ccc(-c4ccc5c(c4)oc4ccc6oc(-c7ccccc7)nc6c45)cc3)c3cccc(-c4cccc5ccccc45)c3)cc2)cc1 ; c1ccc(-c2ccc(N(c3ccc(-c4ccc5c(c4)oc4ccc6oc(-c7ccccc7)nc6c45)cc3)c3ccccc3-c3cccc4ccccc34)cc2)cc1]

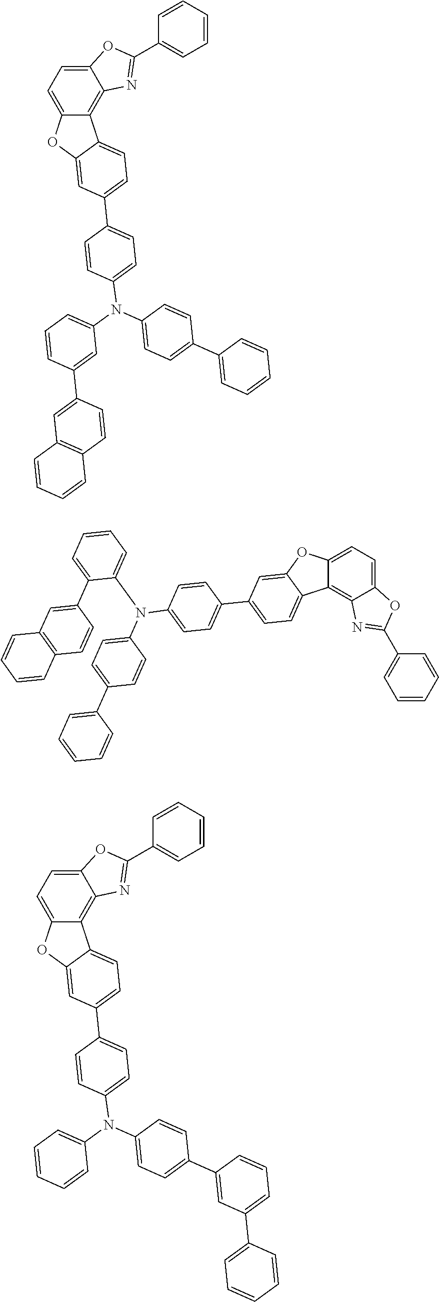 [SMILES: c1ccc(-c2ccc(N(c3ccc(-c4ccc5c(c4)oc4ccc6oc(-c7ccccc7)nc6c45)cc3)c3cccc(-c4ccc5ccccc5c4)c3)cc2)cc1 ; c1ccc(-c2ccc(N(c3ccc(-c4ccc5c(c4)oc4ccc6oc(-c7ccccc7)nc6c45)cc3)c3ccccc3-c3ccc4ccccc4c3)cc2)cc1 ; c1ccc(-c2cccc(-c3ccc(N(c4ccccc4)c4ccc(-c5ccc6c(c5)oc5ccc7oc(-c8ccccc8)nc7c56)cc4)cc3)c2)cc1]

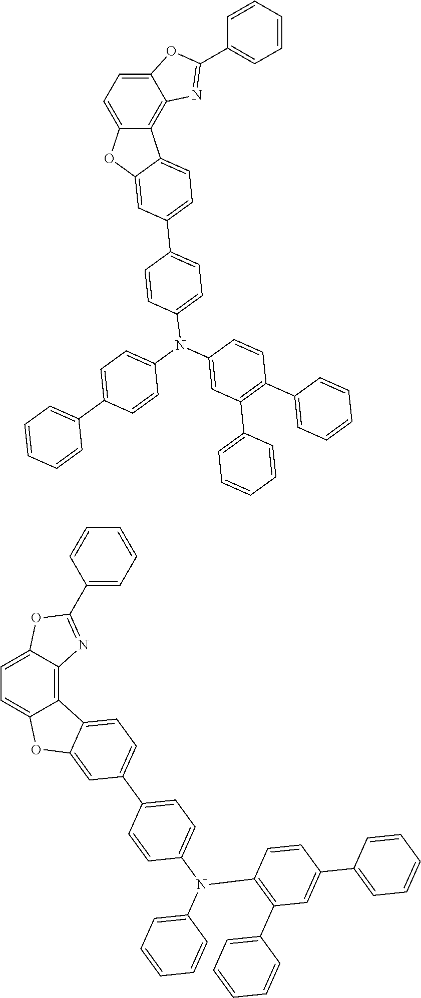 [SMILES: c1ccc(-c2ccc(N(c3ccc(-c4ccc5c(c4)oc4ccc6oc(-c7ccccc7)nc6c45)cc3)c3ccc(-c4ccccc4)c(-c4ccccc4)c3)cc2)cc1 ; c1ccc(-c2ccc(N(c3ccccc3)c3ccc(-c4ccc5c(c4)oc4ccc6oc(-c7ccccc7)nc6c45)cc3)c(-c3ccccc3)c2)cc1]

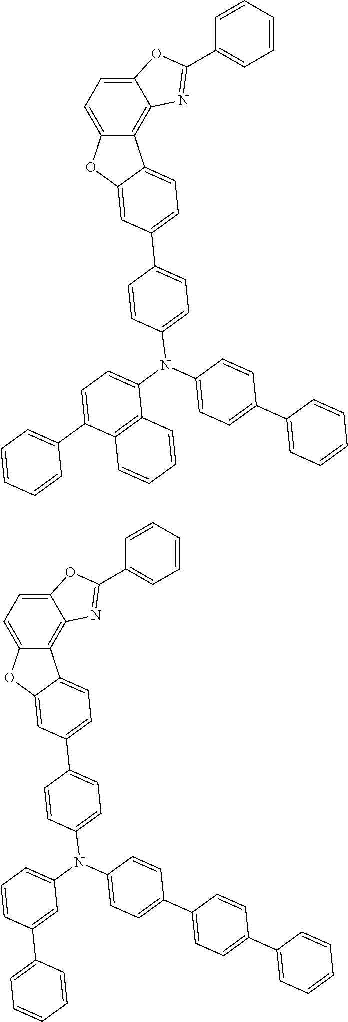

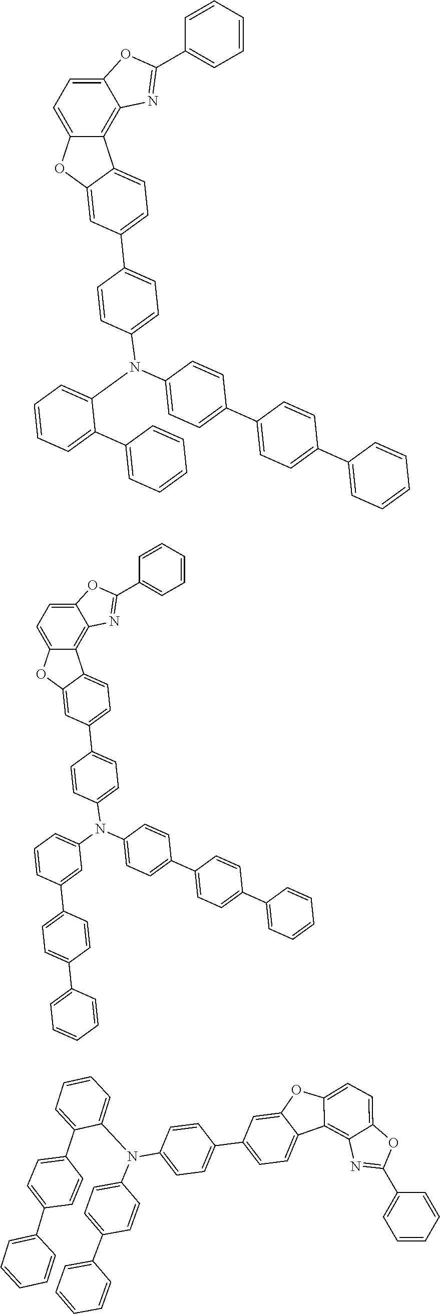

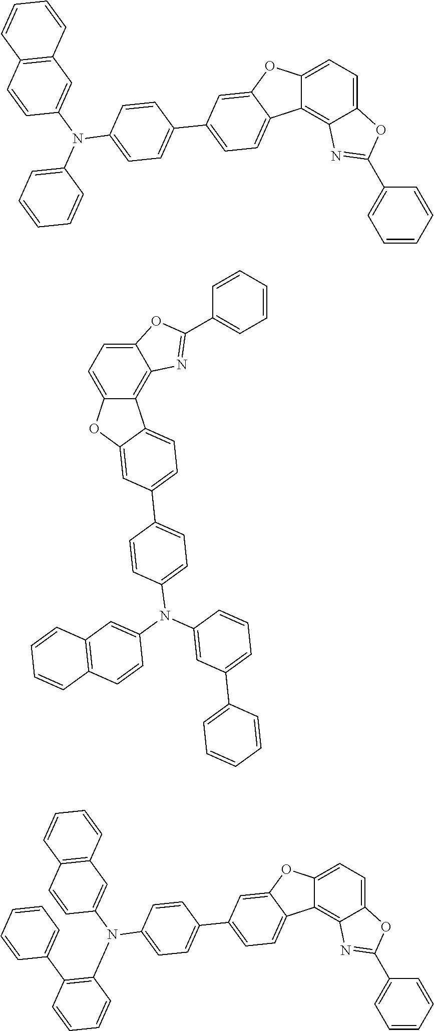 [SMILES: c1ccc(-c2nc3c(ccc4oc5cc(-c6ccc(N(c7ccccc7)c7ccc8ccccc8c7)cc6)ccc5c43)o2)cc1 ; c1ccc(-c2cccc(N(c3ccc(-c4ccc5c(c4)oc4ccc6oc(-c7ccccc7)nc6c45)cc3)c3ccc4ccccc4c3)c2)cc1 ; c1ccc(-c2nc3c(ccc4oc5cc(-c6ccc(N(c7ccc8ccccc8c7)c7ccccc7-c7ccccc7)cc6)ccc5c43)o2)cc1]

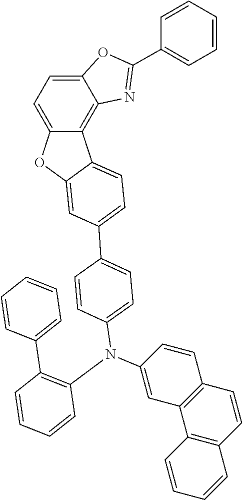 [SMILES: c1ccc(-c2nc3c(ccc4oc5cc(-c6ccc(N(c7ccc8ccc9ccccc9c8c7)c7ccccc7-c7ccccc7)cc6)ccc5c43)o2)cc1]

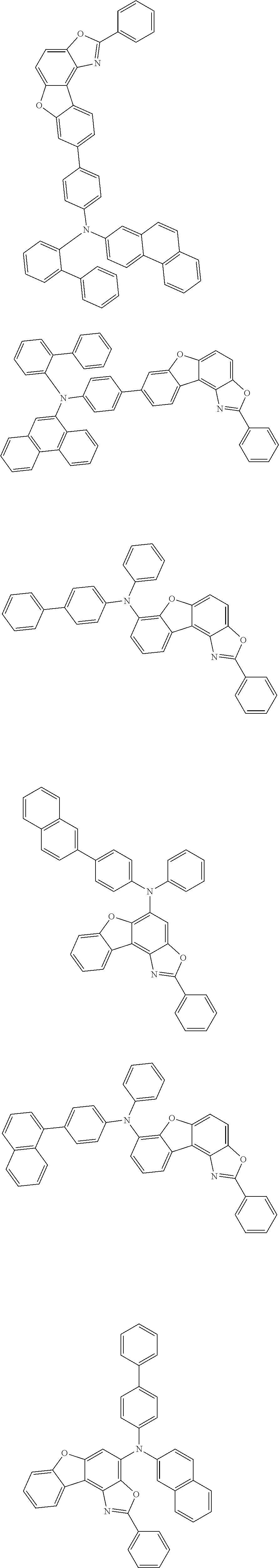

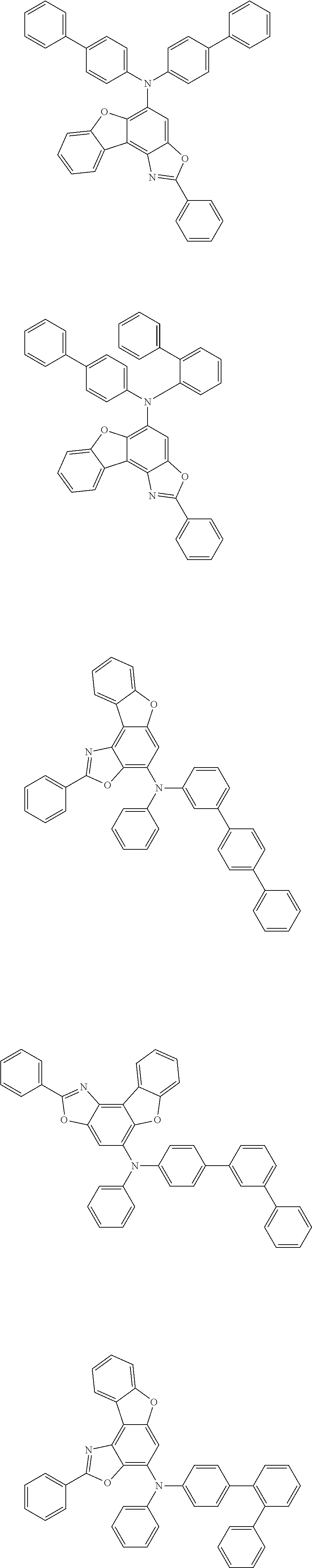 [SMILES: c1ccc(-c2ccc(N(c3ccc(-c4ccccc4)cc3)c3cc4oc(-c5ccccc5)nc4c4c3oc3ccccc34)cc2)cc1 ; c1ccc(-c2ccc(N(c3ccccc3-c3ccccc3)c3cc4oc(-c5ccccc5)nc4c4c3oc3ccccc34)cc2)cc1 ; c1ccc(-c2ccc(-c3cccc(N(c4ccccc4)c4cc5oc6ccccc6c5c5nc(-c6ccccc6)oc45)c3)cc2)cc1 ; c1ccc(-c2cccc(-c3ccc(N(c4ccccc4)c4cc5oc(-c6ccccc6)nc5c5c4oc4ccccc45)cc3)c2)cc1 ; c1ccc(-c2nc3c(o2)c(N(c2ccccc2)c2ccc(-c4ccccc4-c4ccccc4)cc2)cc2oc4ccccc4c23)cc1]

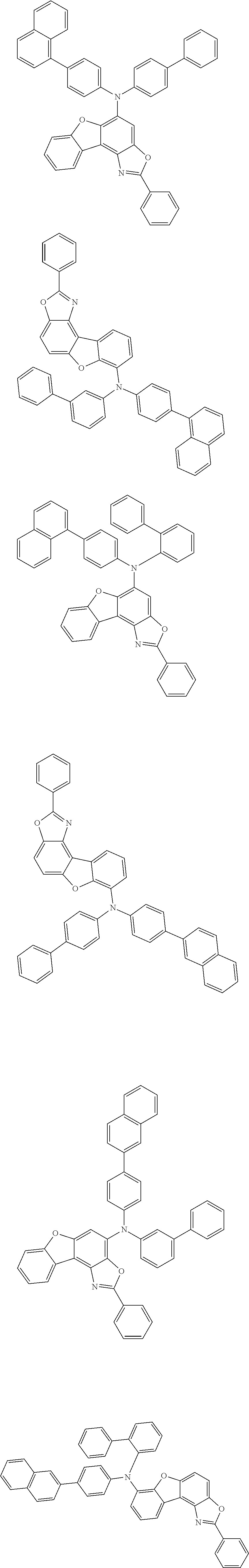

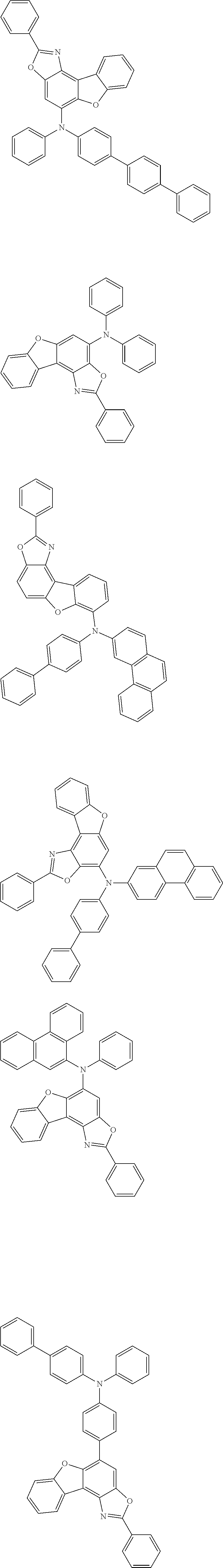

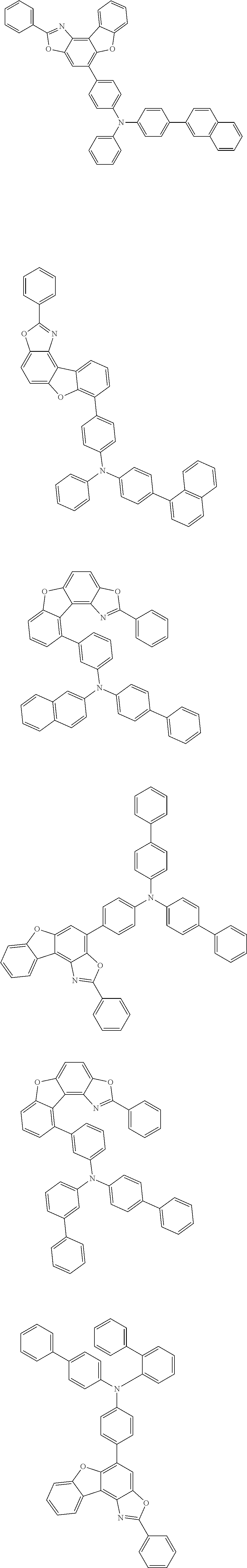

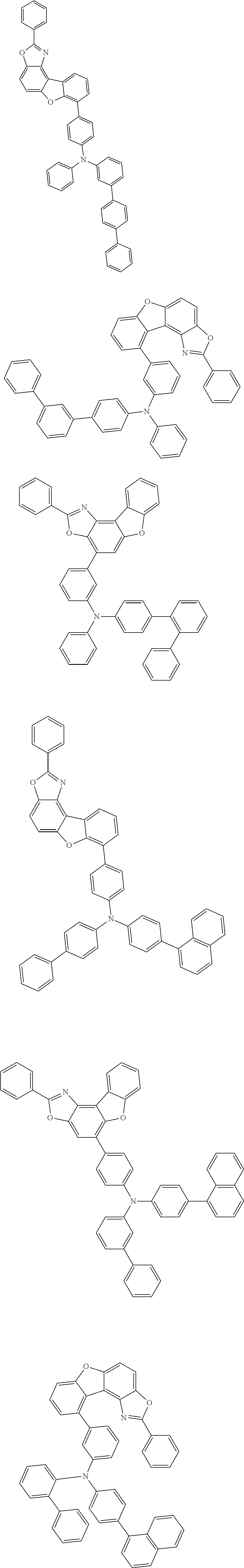

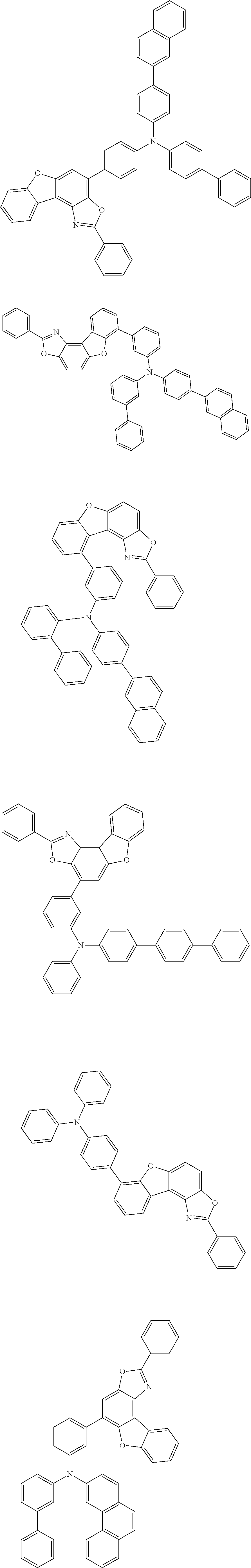

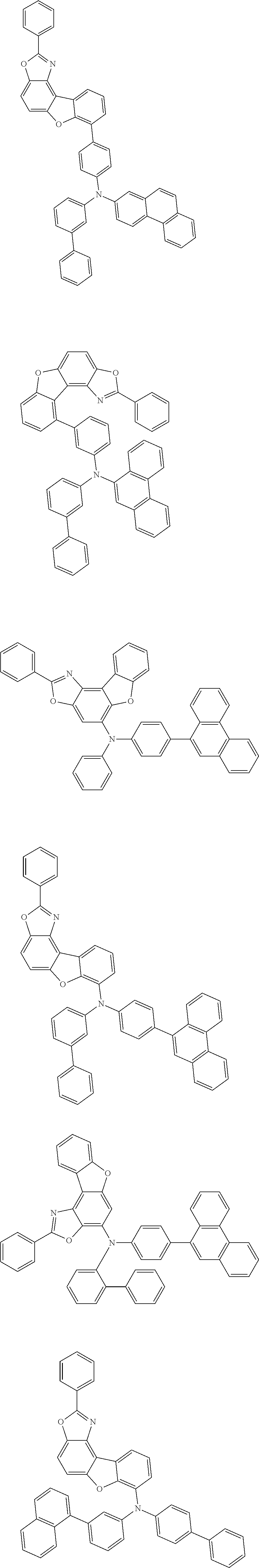

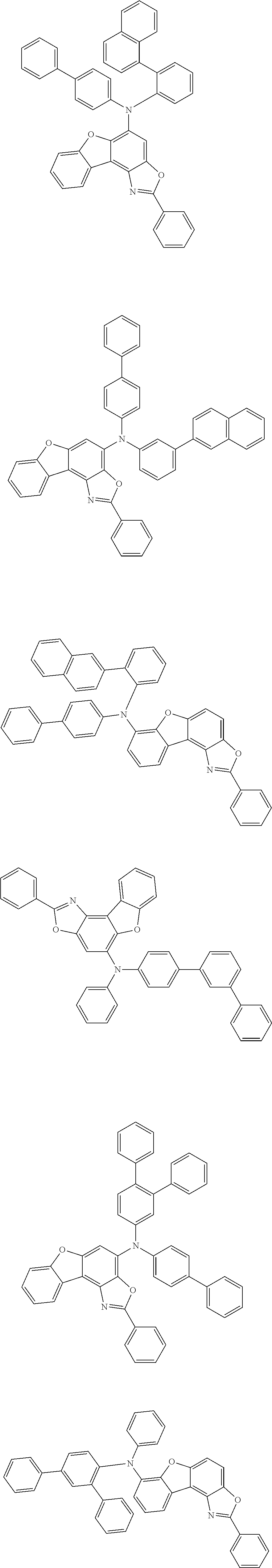

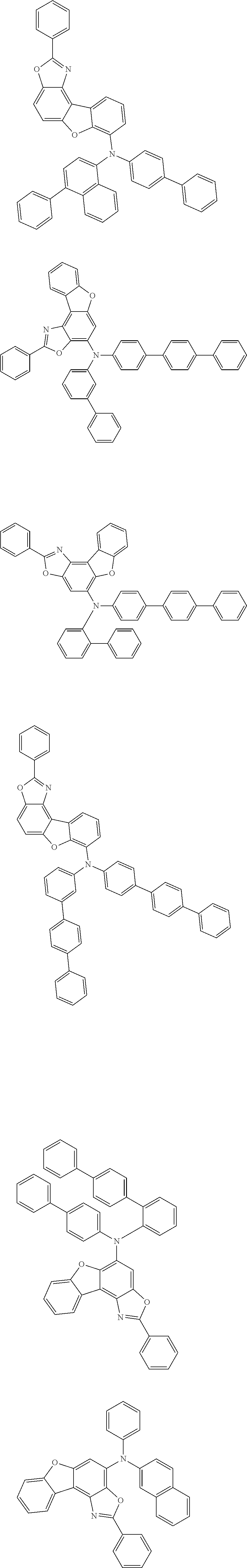

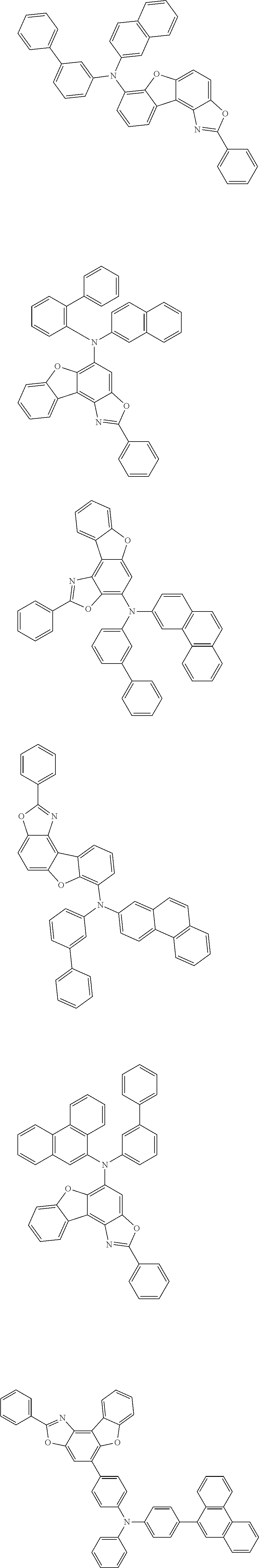 [SMILES: c1ccc(-c2cccc(N(c3ccc4ccccc4c3)c3cccc4c3oc3ccc5oc(-c6ccccc6)nc5c34)c2)cc1 ; c1ccc(-c2nc3c(cc(N(c4ccc5ccccc5c4)c4ccccc4-c4ccccc4)c4oc5ccccc5c43)o2)cc1 ; c1ccc(-c2cccc(N(c3ccc4ccc5ccccc5c4c3)c3cc4oc5ccccc5c4c4nc(-c5ccccc5)oc34)c2)cc1 ; c1ccc(-c2cccc(N(c3ccc4c(ccc5ccccc54)c3)c3cccc4c3oc3ccc5oc(-c6ccccc6)nc5c34)c2)cc1 ; c1ccc(-c2cccc(N(c3cc4ccccc4c4ccccc34)c3cc4oc(-c5ccccc5)nc4c4c3oc3ccccc34)c2)cc1 ; c1ccc(-c2nc3c(cc(-c4ccc(N(c5ccccc5)c5ccc(-c6cc7ccccc7c7ccccc67)cc5)cc4)c4oc5ccccc5c43)o2)cc1]

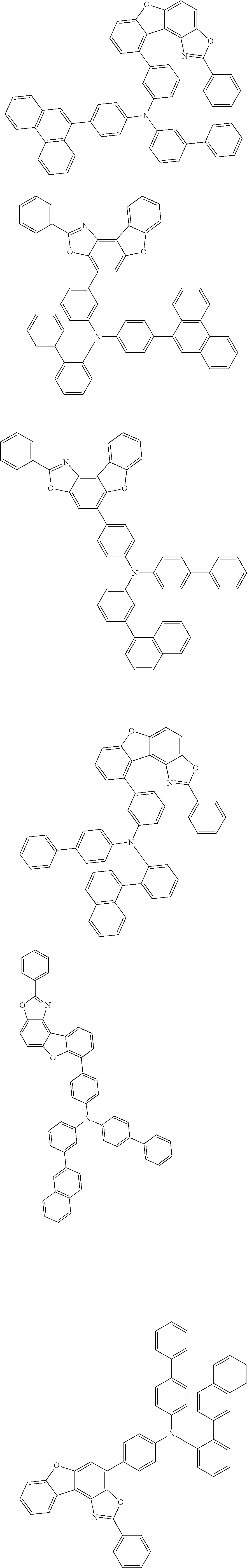

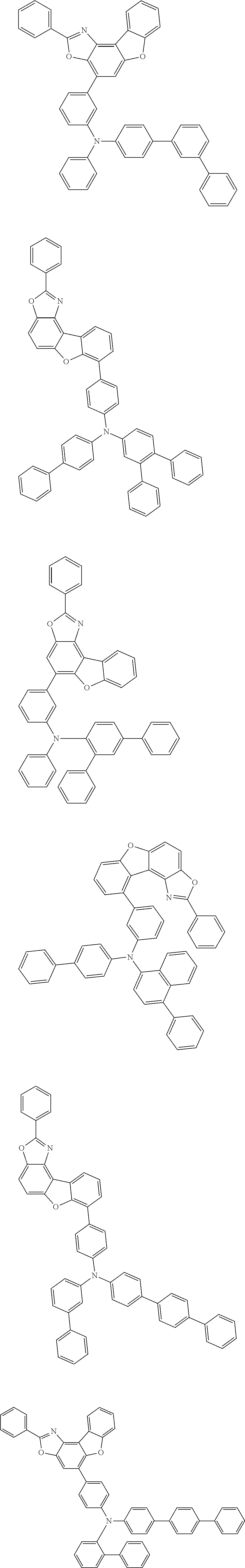

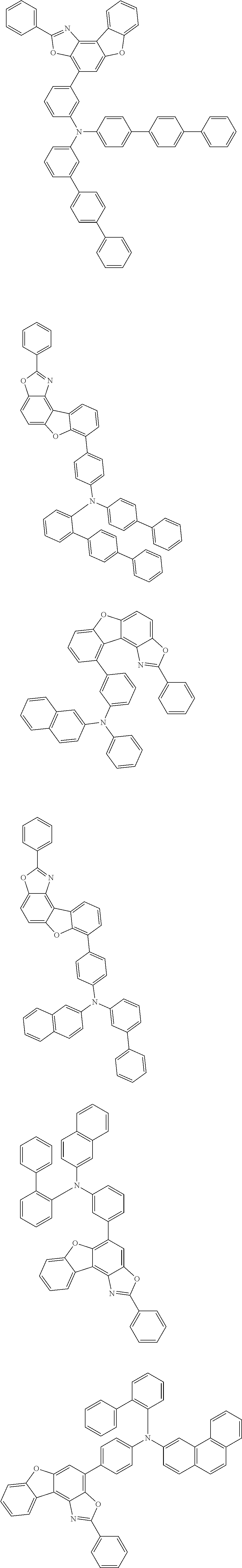

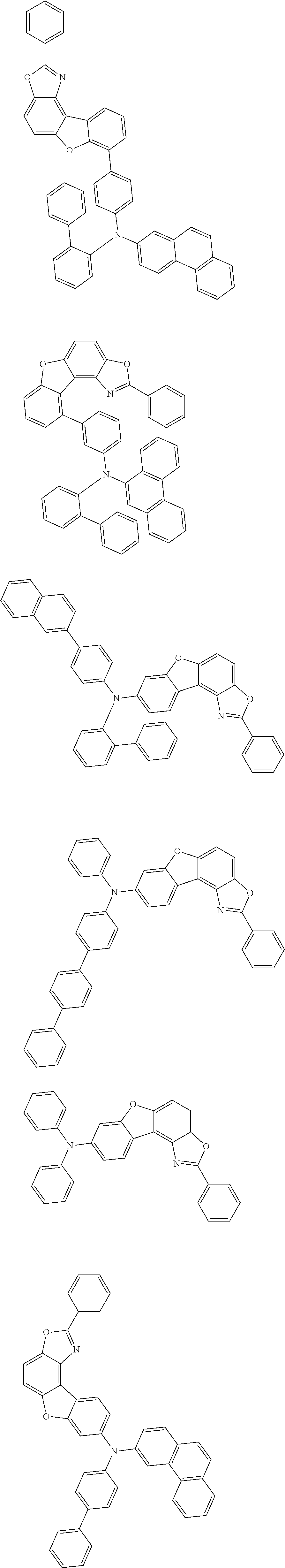

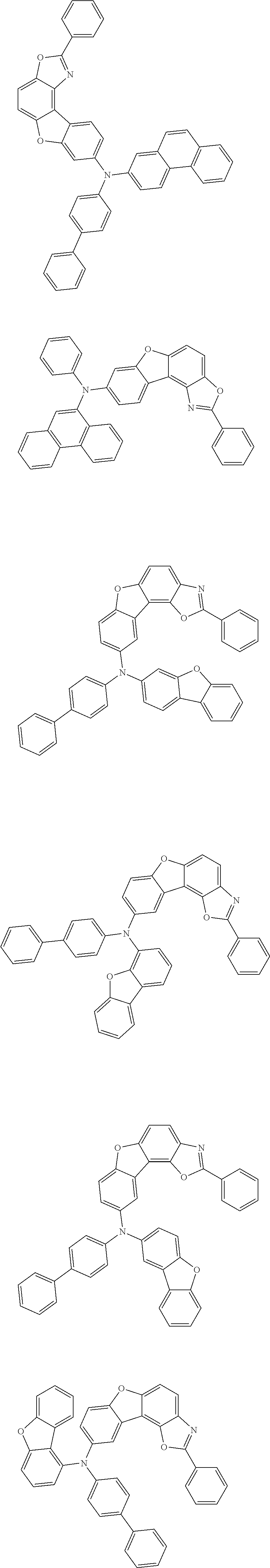 [SMILES: c1ccc(-c2ccc(N(c3ccc4c(ccc5ccccc54)c3)c3ccc4c(c3)oc3ccc5oc(-c6ccccc6)nc5c34)cc2)cc1 ; c1ccc(-c2nc3c(ccc4oc5cc(N(c6ccccc6)c6cc7ccccc7c7ccccc67)ccc5c43)o2)cc1 ; c1ccc(-c2ccc(N(c3ccc4c(c3)oc3ccccc34)c3ccc4oc5ccc6nc(-c7ccccc7)oc6c5c4c3)cc2)cc1 ; c1ccc(-c2ccc(N(c3ccc4oc5ccc6nc(-c7ccccc7)oc6c5c4c3)c3cccc4c3oc3ccccc34)cc2)cc1 ; c1ccc(-c2ccc(N(c3ccc4oc5ccccc5c4c3)c3ccc4oc5ccc6nc(-c7ccccc7)oc6c5c4c3)cc2)cc1 ; c1ccc(-c2ccc(N(c3ccc4oc5ccc6nc(-c7ccccc7)oc6c5c4c3)c3cccc4oc5ccccc5c34)cc2)cc1]

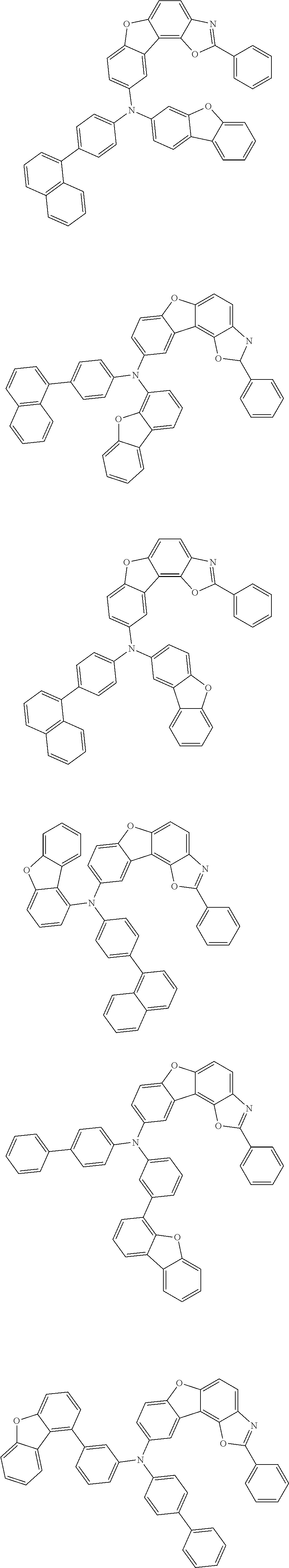

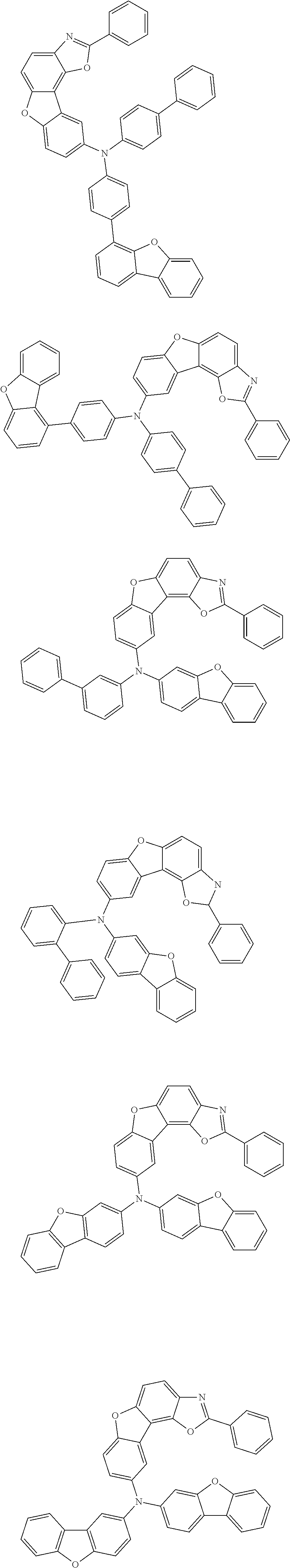

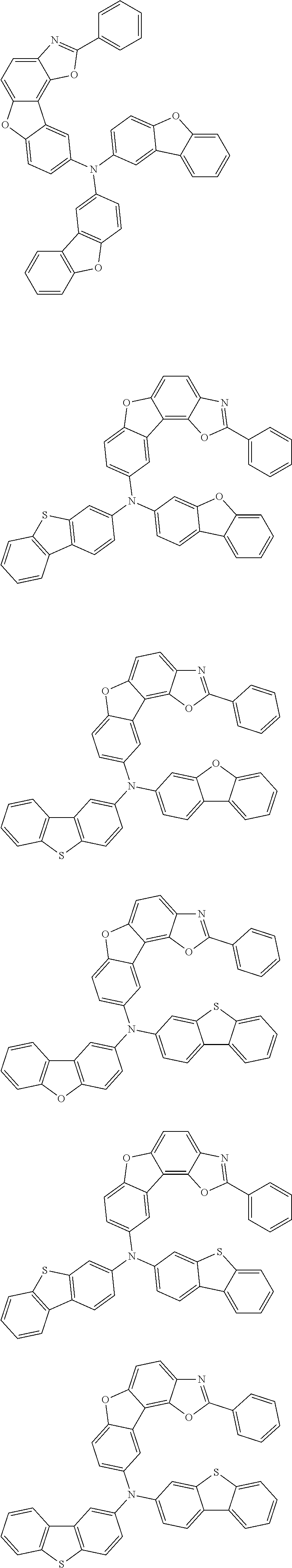 [SMILES: c1ccc(-c2nc3ccc4oc5ccc(N(c6ccc7oc8ccccc8c7c6)c6ccc7oc8ccccc8c7c6)cc5c4c3o2)cc1 ; c1ccc(-c2nc3ccc4oc5ccc(N(c6ccc7c(c6)oc6ccccc67)c6ccc7c(c6)sc6ccccc67)cc5c4c3o2)cc1 ; c1ccc(-c2nc3ccc4oc5ccc(N(c6ccc7c(c6)oc6ccccc67)c6ccc7sc8ccccc8c7c6)cc5c4c3o2)cc1 ; c1ccc(-c2nc3ccc4oc5ccc(N(c6ccc7c(c6)sc6ccccc67)c6ccc7oc8ccccc8c7c6)cc5c4c3o2)cc1 ; c1ccc(-c2nc3ccc4oc5ccc(N(c6ccc7c(c6)sc6ccccc67)c6ccc7c(c6)sc6ccccc67)cc5c4c3o2)cc1 ; c1ccc(-c2nc3ccc4oc5ccc(N(c6ccc7c(c6)sc6ccccc67)c6ccc7sc8ccccc8c7c6)cc5c4c3o2)cc1]

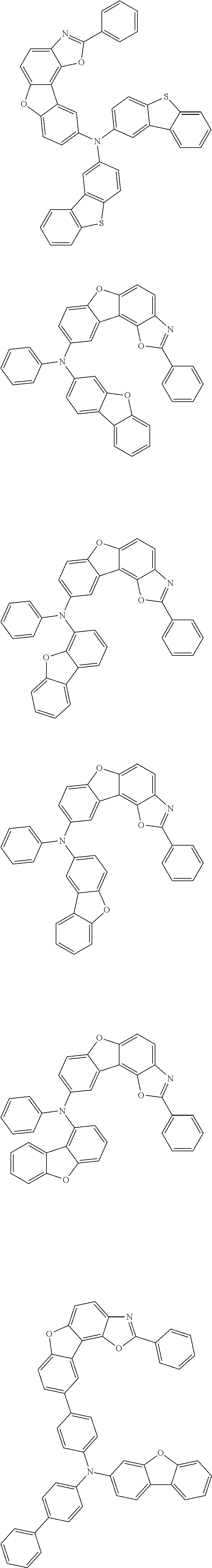 [SMILES: c1ccc(-c2nc3ccc4oc5ccc(N(c6ccc7sc8ccccc8c7c6)c6ccc7sc8ccccc8c7c6)cc5c4c3o2)cc1 ; c1ccc(-c2nc3ccc4oc5ccc(N(c6ccccc6)c6ccc7c(c6)oc6ccccc67)cc5c4c3o2)cc1 ; c1ccc(-c2nc3ccc4oc5ccc(N(c6ccccc6)c6cccc7c6oc6ccccc67)cc5c4c3o2)cc1 ; c1ccc(-c2nc3ccc4oc5ccc(N(c6ccccc6)c6ccc7oc8ccccc8c7c6)cc5c4c3o2)cc1 ; c1ccc(-c2nc3ccc4oc5ccc(N(c6ccccc6)c6cccc7oc8ccccc8c67)cc5c4c3o2)cc1 ; c1ccc(-c2ccc(N(c3ccc(-c4ccc5oc6ccc7nc(-c8ccccc8)oc7c6c5c4)cc3)c3ccc4c(c3)oc3ccccc34)cc2)cc1]

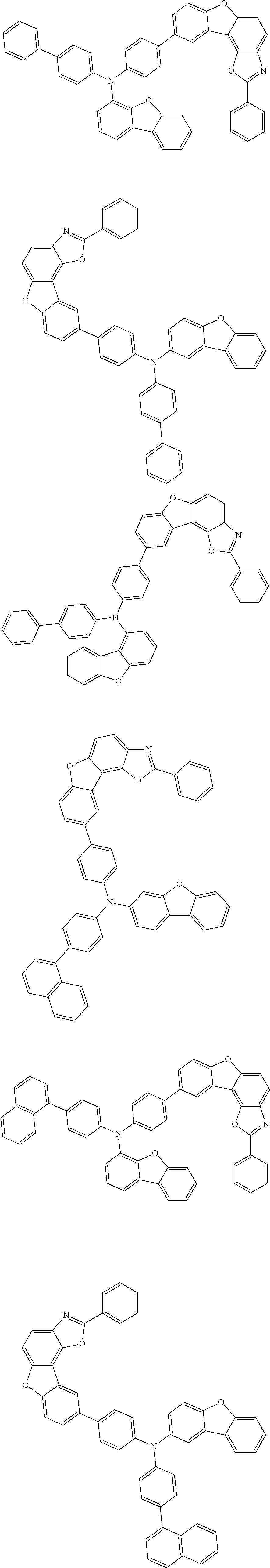

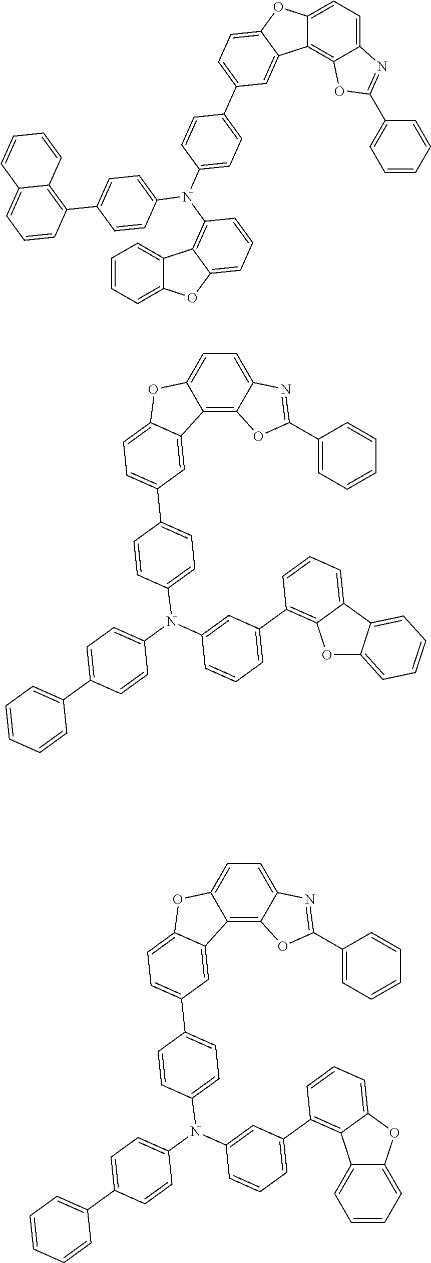 [SMILES: c1ccc(-c2nc3ccc4oc5ccc(-c6ccc(N(c7ccc(-c8cccc9ccccc89)cc7)c7cccc8oc9ccccc9c78)cc6)cc5c4c3o2)cc1 ; c1ccc(-c2ccc(N(c3ccc(-c4ccc5oc6ccc7nc(-c8ccccc8)oc7c6c5c4)cc3)c3cccc(-c4cccc5c4oc4ccccc45)c3)cc2)cc1 ; c1ccc(-c2ccc(N(c3ccc(-c4ccc5oc6ccc7nc(-c8ccccc8)oc7c6c5c4)cc3)c3cccc(-c4cccc5oc6ccccc6c45)c3)cc2)cc1]

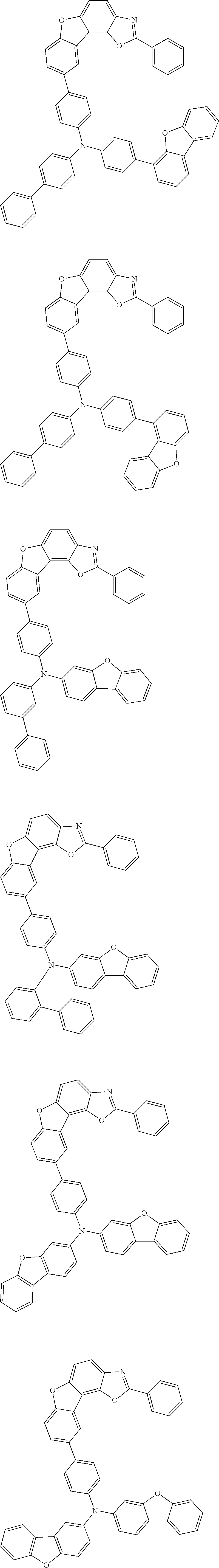

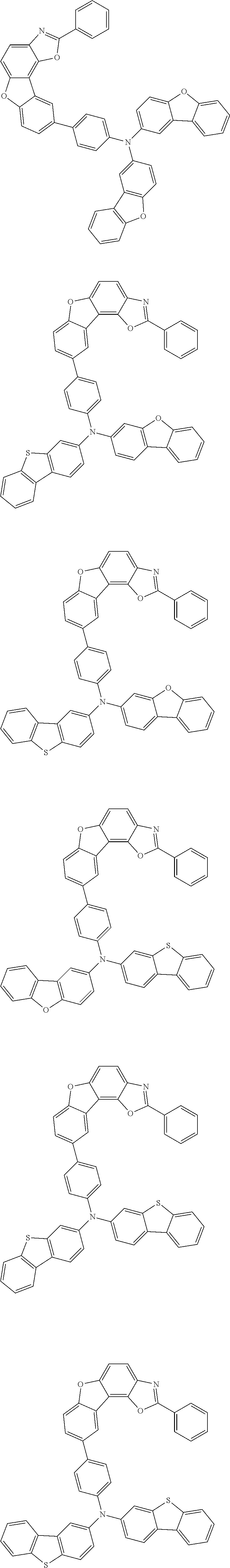

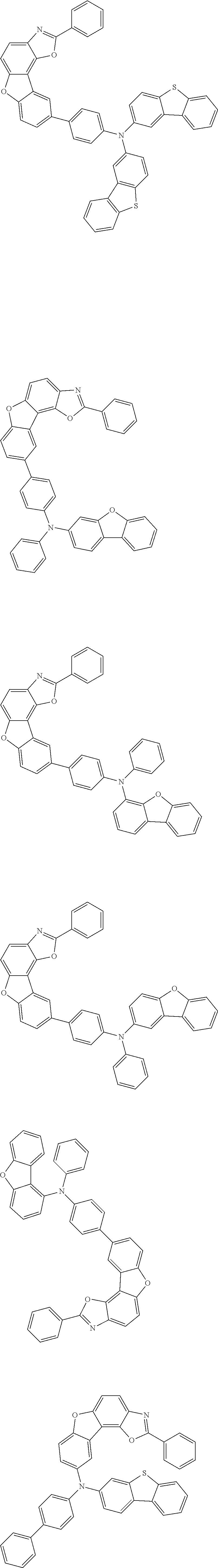 [SMILES: c1ccc(-c2nc3ccc4oc5ccc(-c6ccc(N(c7ccc8sc9ccccc9c8c7)c7ccc8sc9ccccc9c8c7)cc6)cc5c4c3o2)cc1 ; c1ccc(-c2nc3ccc4oc5ccc(-c6ccc(N(c7ccccc7)c7ccc8c(c7)oc7ccccc78)cc6)cc5c4c3o2)cc1 ; c1ccc(-c2nc3ccc4oc5ccc(-c6ccc(N(c7ccccc7)c7cccc8c7oc7ccccc78)cc6)cc5c4c3o2)cc1 ; c1ccc(-c2nc3ccc4oc5ccc(-c6ccc(N(c7ccccc7)c7ccc8oc9ccccc9c8c7)cc6)cc5c4c3o2)cc1 ; c1ccc(-c2nc3ccc4oc5ccc(-c6ccc(N(c7ccccc7)c7cccc8oc9ccccc9c78)cc6)cc5c4c3o2)cc1 ; c1ccc(-c2ccc(N(c3ccc4c(c3)sc3ccccc34)c3ccc4oc5ccc6nc(-c7ccccc7)oc6c5c4c3)cc2)cc1]

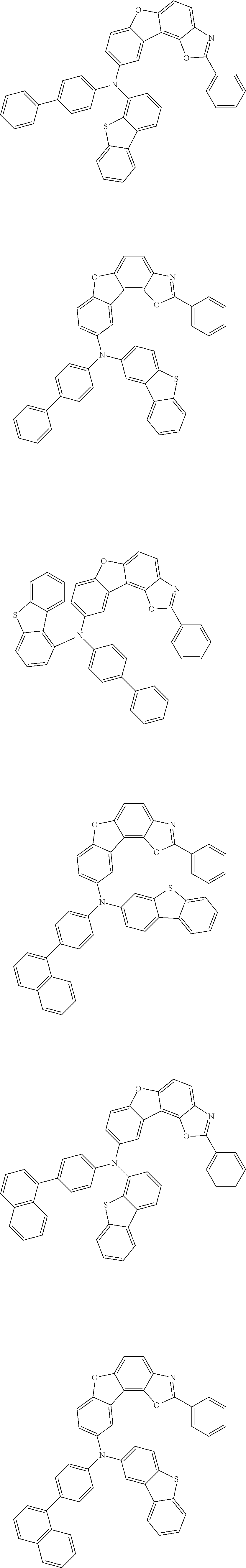

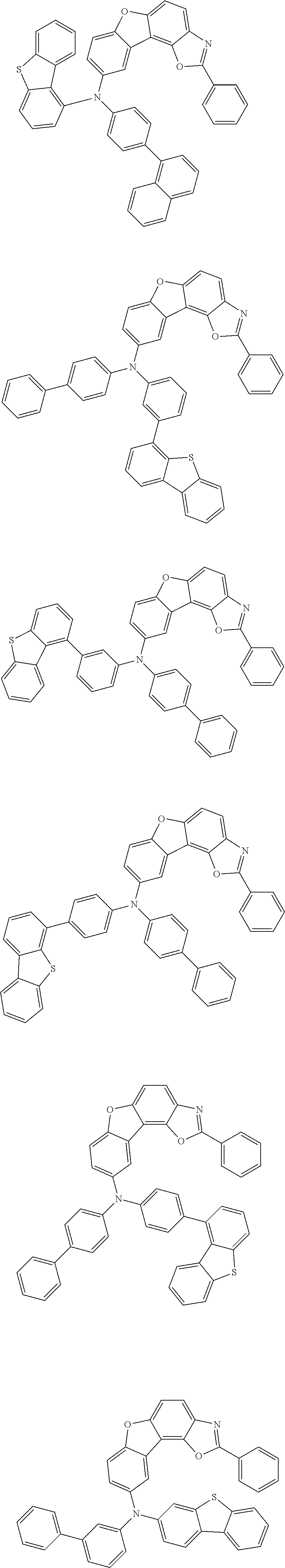

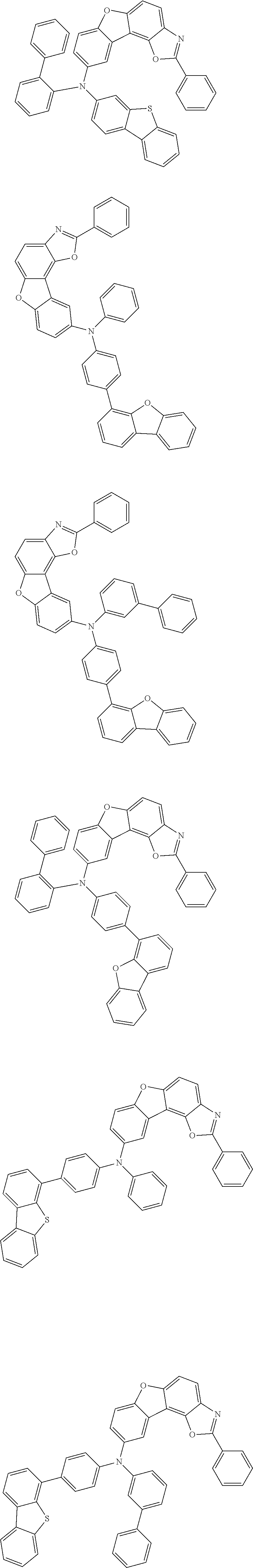

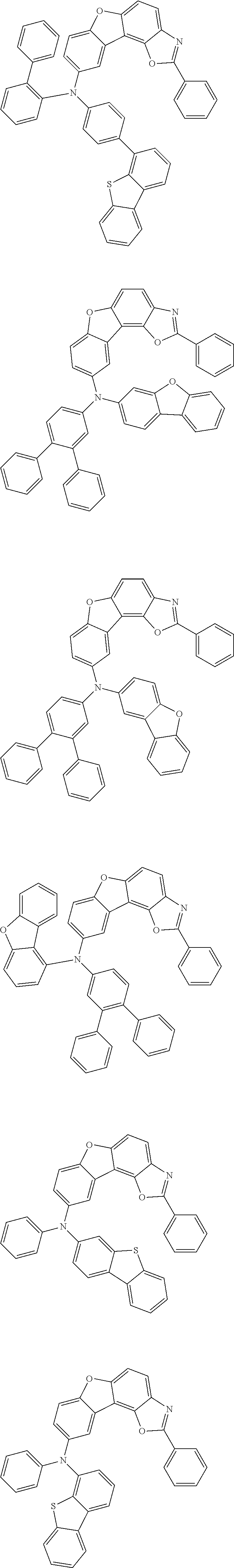

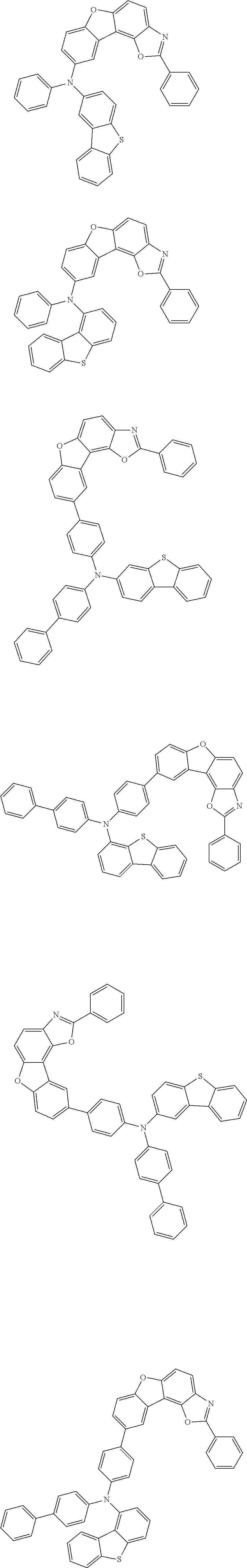

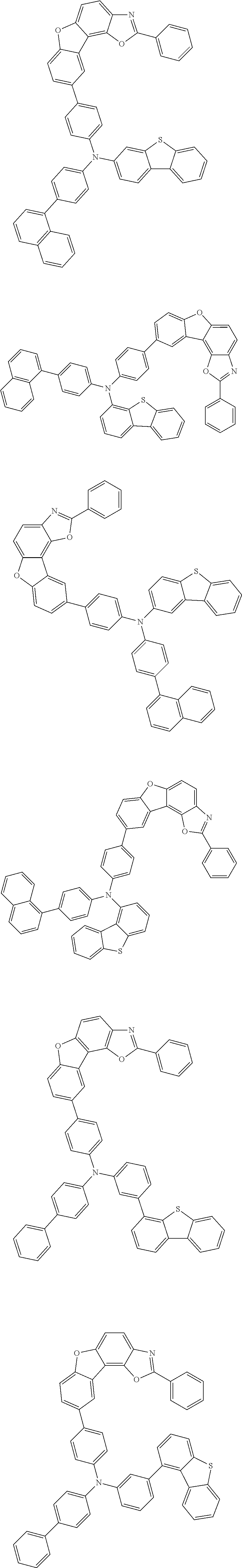

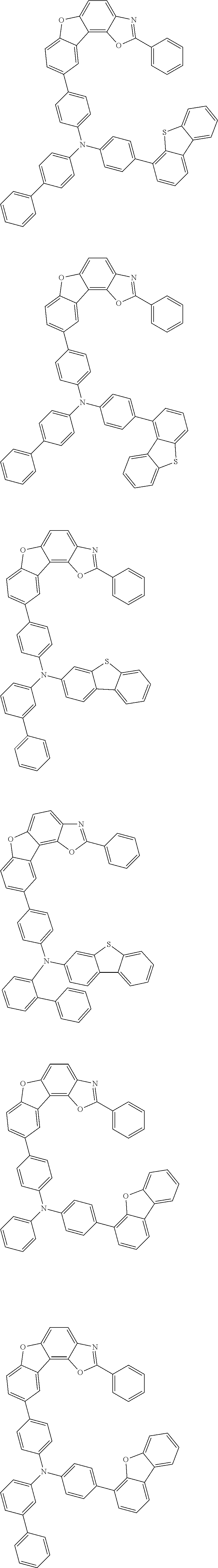

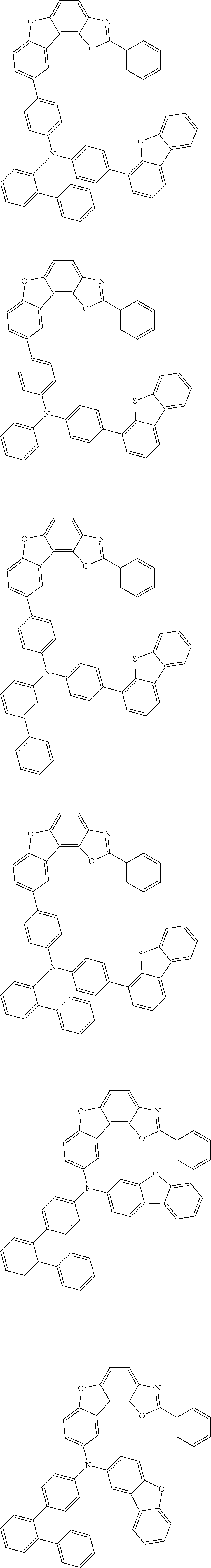

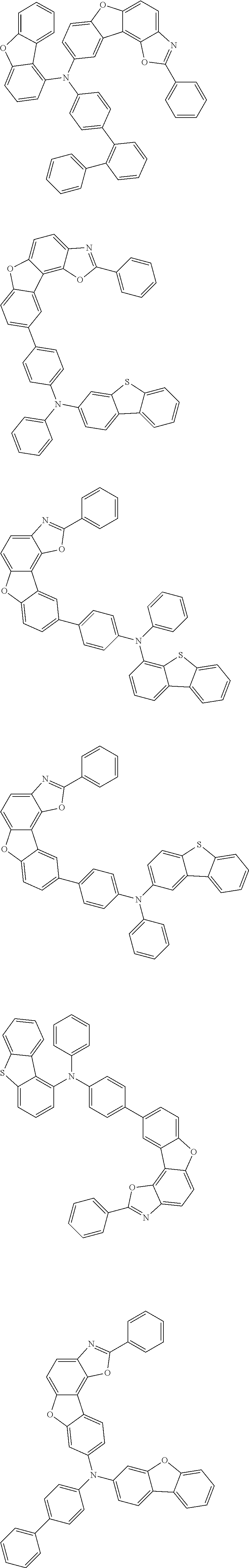 [SMILES: c1ccc(-c2nc3ccc4oc5ccc(N(c6ccc(-c7ccccc7-c7ccccc7)cc6)c6cccc7oc8ccccc8c67)cc5c4c3o2)cc1 ; c1ccc(-c2nc3ccc4oc5ccc(-c6ccc(N(c7ccccc7)c7ccc8c(c7)sc7ccccc78)cc6)cc5c4c3o2)cc1 ; c1ccc(-c2nc3ccc4oc5ccc(-c6ccc(N(c7ccccc7)c7cccc8c7sc7ccccc78)cc6)cc5c4c3o2)cc1 ; c1ccc(-c2nc3ccc4oc5ccc(-c6ccc(N(c7ccccc7)c7ccc8sc9ccccc9c8c7)cc6)cc5c4c3o2)cc1 ; c1ccc(-c2nc3ccc4oc5ccc(-c6ccc(N(c7ccccc7)c7cccc8sc9ccccc9c78)cc6)cc5c4c3o2)cc1 ; c1ccc(-c2ccc(N(c3ccc4c(c3)oc3ccccc34)c3ccc4c(c3)oc3ccc5nc(-c6ccccc6)oc5c34)cc2)cc1]

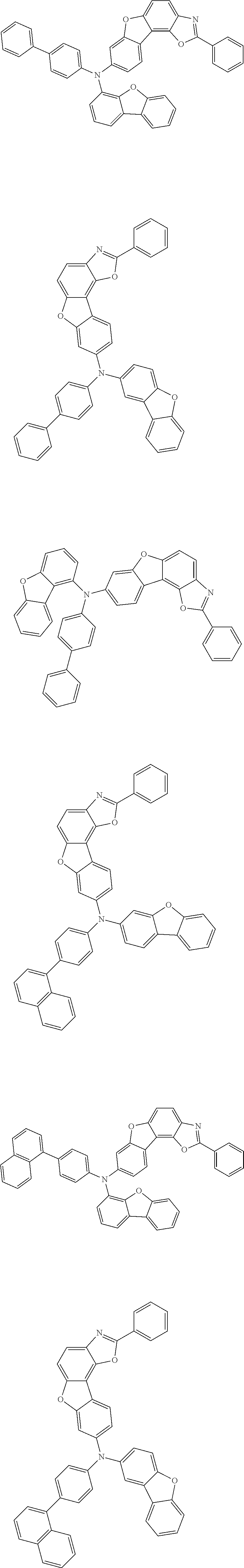

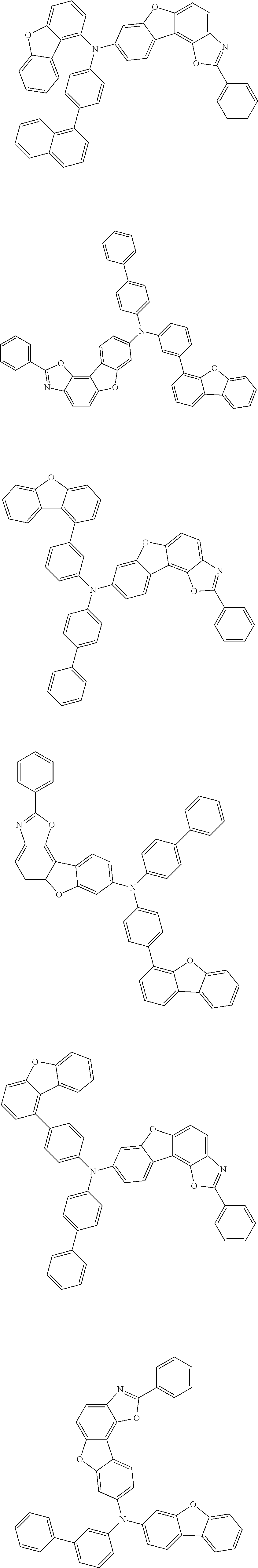

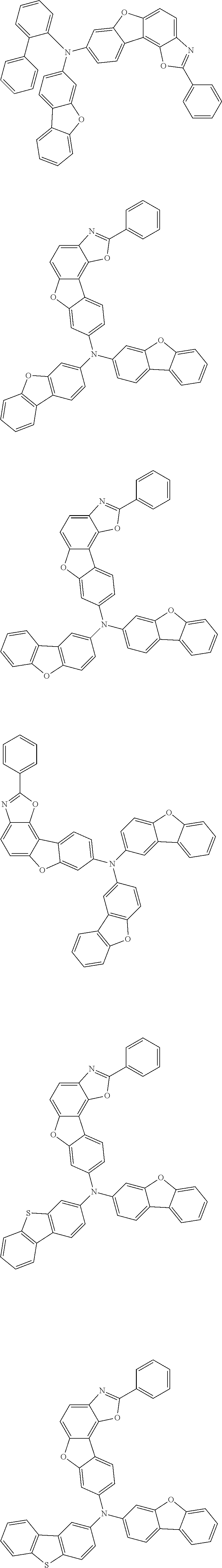 [SMILES: c1ccc(-c2nc3ccc4oc5cc(N(c6ccc7c(c6)oc6ccccc67)c6ccccc6-c6ccccc6)ccc5c4c3o2)cc1 ; c1ccc(-c2nc3ccc4oc5cc(N(c6ccc7c(c6)oc6ccccc67)c6ccc7c(c6)oc6ccccc67)ccc5c4c3o2)cc1 ; c1ccc(-c2nc3ccc4oc5cc(N(c6ccc7c(c6)oc6ccccc67)c6ccc7oc8ccccc8c7c6)ccc5c4c3o2)cc1 ; c1ccc(-c2nc3ccc4oc5cc(N(c6ccc7oc8ccccc8c7c6)c6ccc7oc8ccccc8c7c6)ccc5c4c3o2)cc1 ; c1ccc(-c2nc3ccc4oc5cc(N(c6ccc7c(c6)oc6ccccc67)c6ccc7c(c6)sc6ccccc67)ccc5c4c3o2)cc1 ; c1ccc(-c2nc3ccc4oc5cc(N(c6ccc7c(c6)oc6ccccc67)c6ccc7sc8ccccc8c7c6)ccc5c4c3o2)cc1]

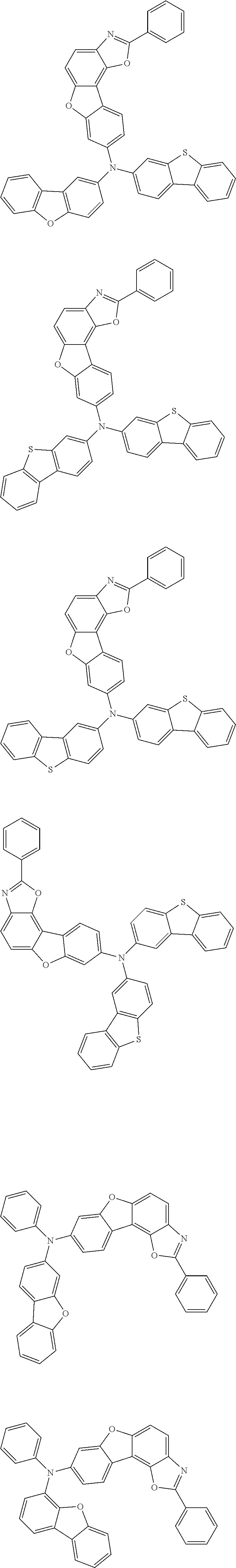 [SMILES: c1ccc(-c2nc3ccc4oc5cc(N(c6ccc7c(c6)sc6ccccc67)c6ccc7oc8ccccc8c7c6)ccc5c4c3o2)cc1 ; c1ccc(-c2nc3ccc4oc5cc(N(c6ccc7c(c6)sc6ccccc67)c6ccc7c(c6)sc6ccccc67)ccc5c4c3o2)cc1 ; c1ccc(-c2nc3ccc4oc5cc(N(c6ccc7c(c6)sc6ccccc67)c6ccc7sc8ccccc8c7c6)ccc5c4c3o2)cc1 ; c1ccc(-c2nc3ccc4oc5cc(N(c6ccc7sc8ccccc8c7c6)c6ccc7sc8ccccc8c7c6)ccc5c4c3o2)cc1 ; c1ccc(-c2nc3ccc4oc5cc(N(c6ccccc6)c6ccc7c(c6)oc6ccccc67)ccc5c4c3o2)cc1 ; c1ccc(-c2nc3ccc4oc5cc(N(c6ccccc6)c6cccc7c6oc6ccccc67)ccc5c4c3o2)cc1]

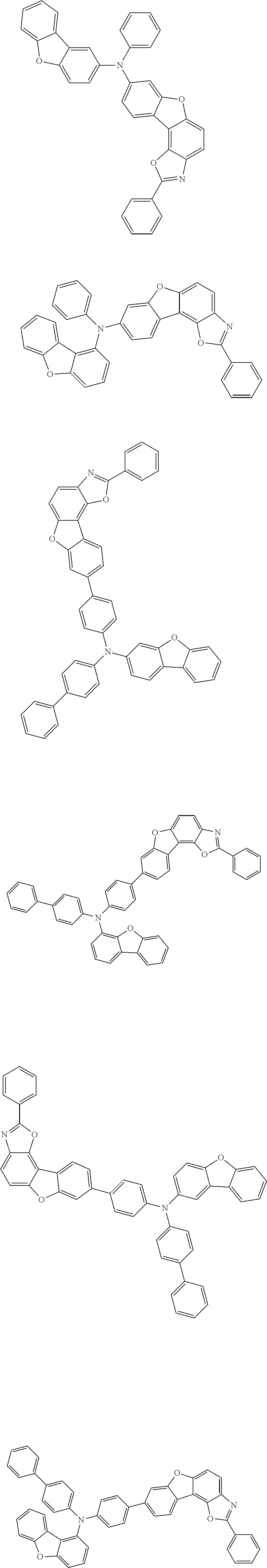

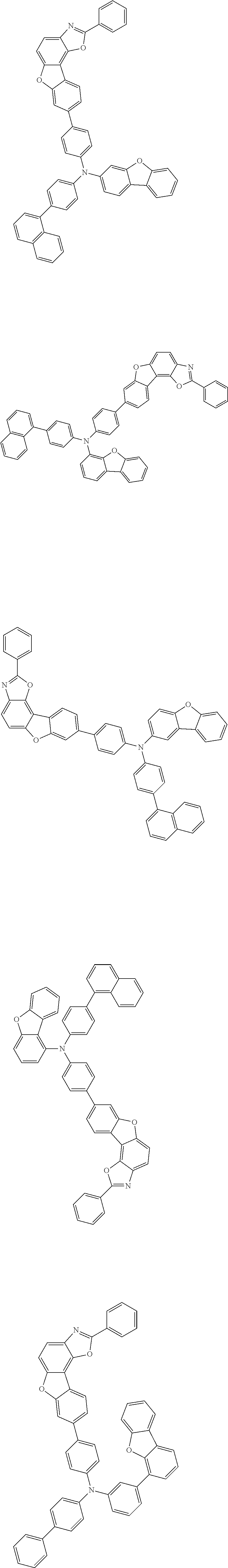 [SMILES: c1ccc(-c2nc3ccc4oc5cc(-c6ccc(N(c7ccc(-c8cccc9ccccc89)cc7)c7ccc8c(c7)oc7ccccc78)cc6)ccc5c4c3o2)cc1 ; c1ccc(-c2nc3ccc4oc5cc(-c6ccc(N(c7ccc(-c8cccc9ccccc89)cc7)c7cccc8c7oc7ccccc78)cc6)ccc5c4c3o2)cc1 ; c1ccc(-c2nc3ccc4oc5cc(-c6ccc(N(c7ccc(-c8cccc9ccccc89)cc7)c7ccc8oc9ccccc9c8c7)cc6)ccc5c4c3o2)cc1 ; c1ccc(-c2nc3ccc4oc5cc(-c6ccc(N(c7ccc(-c8cccc9ccccc89)cc7)c7cccc8oc9ccccc9c78)cc6)ccc5c4c3o2)cc1 ; c1ccc(-c2ccc(N(c3ccc(-c4ccc5c(c4)oc4ccc6nc(-c7ccccc7)oc6c45)cc3)c3cccc(-c4cccc5c4oc4ccccc45)c3)cc2)cc1]

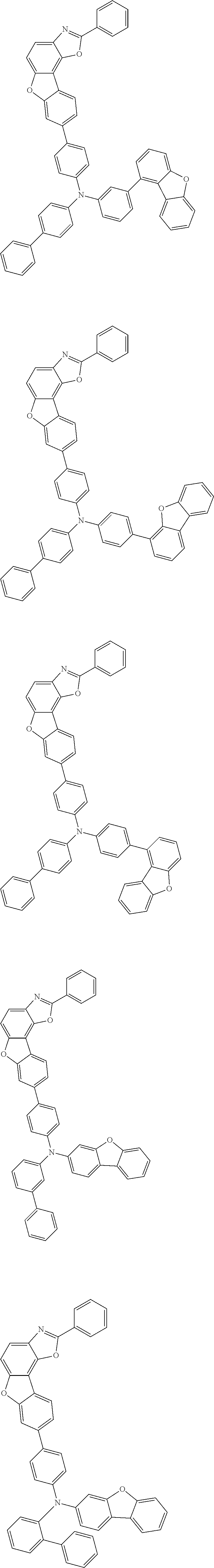 [SMILES: c1ccc(-c2ccc(N(c3ccc(-c4ccc5c(c4)oc4ccc6nc(-c7ccccc7)oc6c45)cc3)c3cccc(-c4cccc5oc6ccccc6c45)c3)cc2)cc1 ; c1ccc(-c2ccc(N(c3ccc(-c4ccc5c(c4)oc4ccc6nc(-c7ccccc7)oc6c45)cc3)c3ccc(-c4cccc5c4oc4ccccc45)cc3)cc2)cc1 ; c1ccc(-c2ccc(N(c3ccc(-c4ccc5c(c4)oc4ccc6nc(-c7ccccc7)oc6c45)cc3)c3ccc(-c4cccc5oc6ccccc6c45)cc3)cc2)cc1 ; c1ccc(-c2cccc(N(c3ccc(-c4ccc5c(c4)oc4ccc6nc(-c7ccccc7)oc6c45)cc3)c3ccc4c(c3)oc3ccccc34)c2)cc1 ; c1ccc(-c2nc3ccc4oc5cc(-c6ccc(N(c7ccc8c(c7)oc7ccccc78)c7ccccc7-c7ccccc7)cc6)ccc5c4c3o2)cc1]

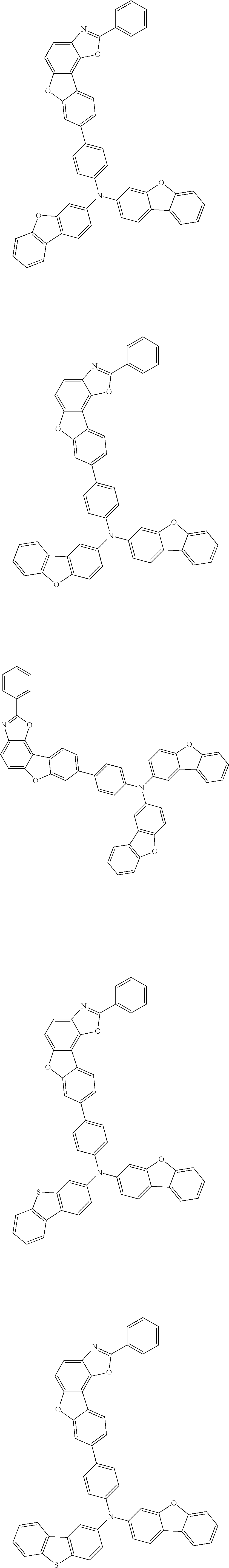 [SMILES: c1ccc(-c2nc3ccc4oc5cc(-c6ccc(N(c7ccc8c(c7)oc7ccccc78)c7ccc8c(c7)oc7ccccc78)cc6)ccc5c4c3o2)cc1 ; c1ccc(-c2nc3ccc4oc5cc(-c6ccc(N(c7ccc8c(c7)oc7ccccc78)c7ccc8oc9ccccc9c8c7)cc6)ccc5c4c3o2)cc1 ; c1ccc(-c2nc3ccc4oc5cc(-c6ccc(N(c7ccc8oc9ccccc9c8c7)c7ccc8oc9ccccc9c8c7)cc6)ccc5c4c3o2)cc1 ; c1ccc(-c2nc3ccc4oc5cc(-c6ccc(N(c7ccc8c(c7)oc7ccccc78)c7ccc8c(c7)sc7ccccc78)cc6)ccc5c4c3o2)cc1 ; c1ccc(-c2nc3ccc4oc5cc(-c6ccc(N(c7ccc8c(c7)oc7ccccc78)c7ccc8sc9ccccc9c8c7)cc6)ccc5c4c3o2)cc1]

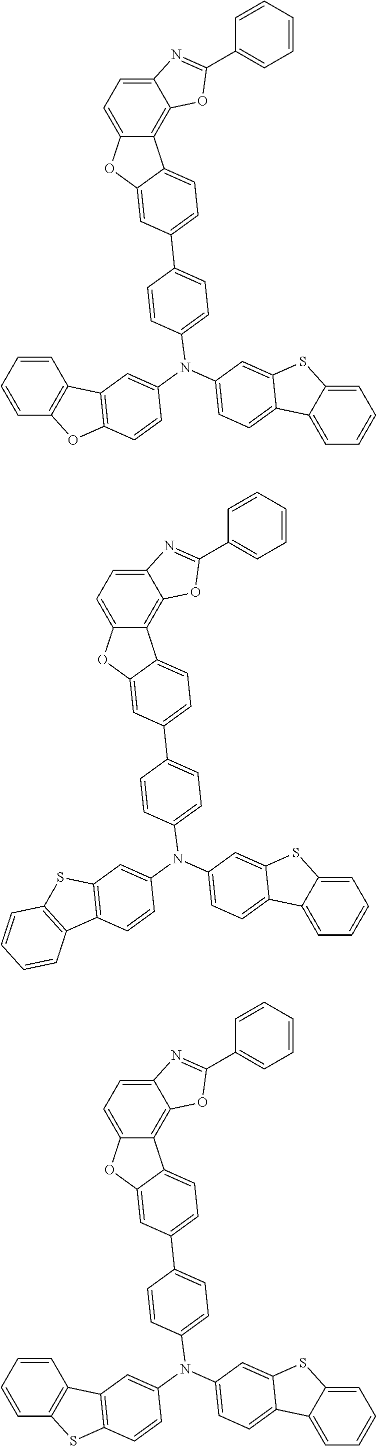 [SMILES: c1ccc(-c2nc3ccc4oc5cc(-c6ccc(N(c7ccc8c(c7)sc7ccccc78)c7ccc8oc9ccccc9c8c7)cc6)ccc5c4c3o2)cc1 ; c1ccc(-c2nc3ccc4oc5cc(-c6ccc(N(c7ccc8c(c7)sc7ccccc78)c7ccc8c(c7)sc7ccccc78)cc6)ccc5c4c3o2)cc1 ; c1ccc(-c2nc3ccc4oc5cc(-c6ccc(N(c7ccc8c(c7)sc7ccccc78)c7ccc8sc9ccccc9c8c7)cc6)ccc5c4c3o2)cc1]

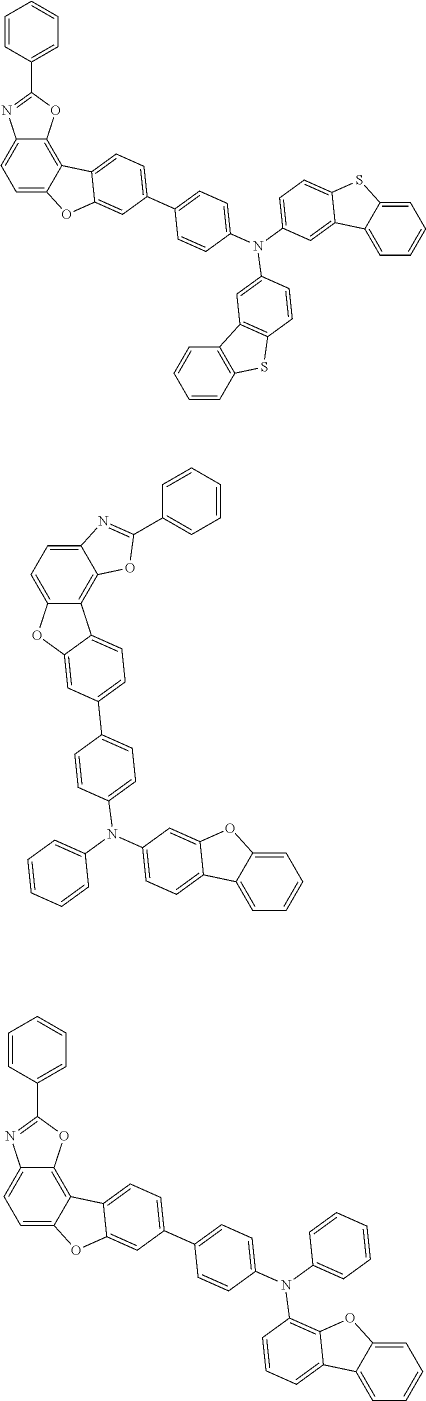 [SMILES: c1ccc(-c2nc3ccc4oc5cc(-c6ccc(N(c7ccc8sc9ccccc9c8c7)c7ccc8sc9ccccc9c8c7)cc6)ccc5c4c3o2)cc1 ; c1ccc(-c2nc3ccc4oc5cc(-c6ccc(N(c7ccccc7)c7ccc8c(c7)oc7ccccc78)cc6)ccc5c4c3o2)cc1 ; c1ccc(-c2nc3ccc4oc5cc(-c6ccc(N(c7ccccc7)c7cccc8c7oc7ccccc78)cc6)ccc5c4c3o2)cc1]

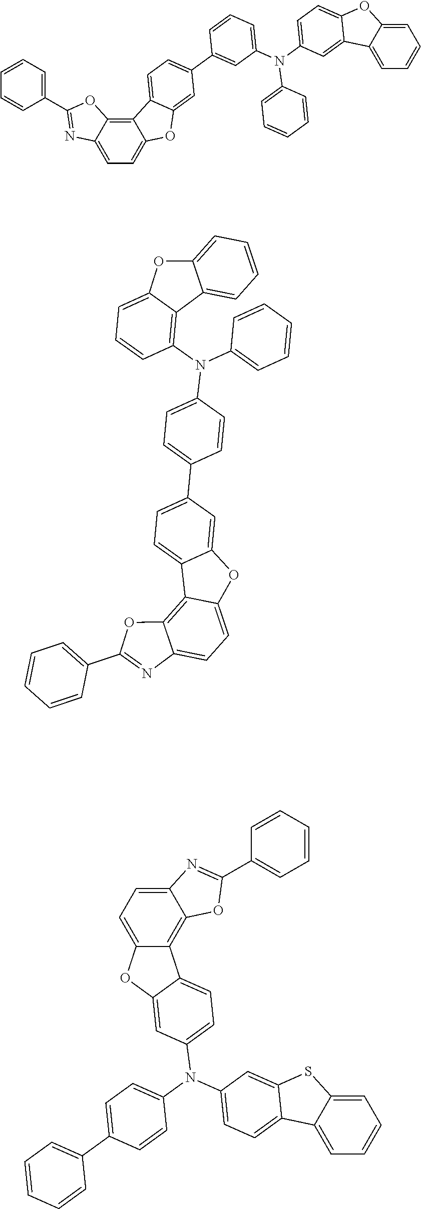 [SMILES: c1ccc(-c2nc3ccc4oc5cc(-c6cccc(N(c7ccccc7)c7ccc8oc9ccccc9c8c7)c6)ccc5c4c3o2)cc1 ; c1ccc(-c2nc3ccc4oc5cc(-c6ccc(N(c7ccccc7)c7cccc8oc9ccccc9c78)cc6)ccc5c4c3o2)cc1 ; c1ccc(-c2ccc(N(c3ccc4c(c3)oc3ccc5nc(-c6ccccc6)oc5c34)c3ccc4c(c3)sc3ccccc34)cc2)cc1]

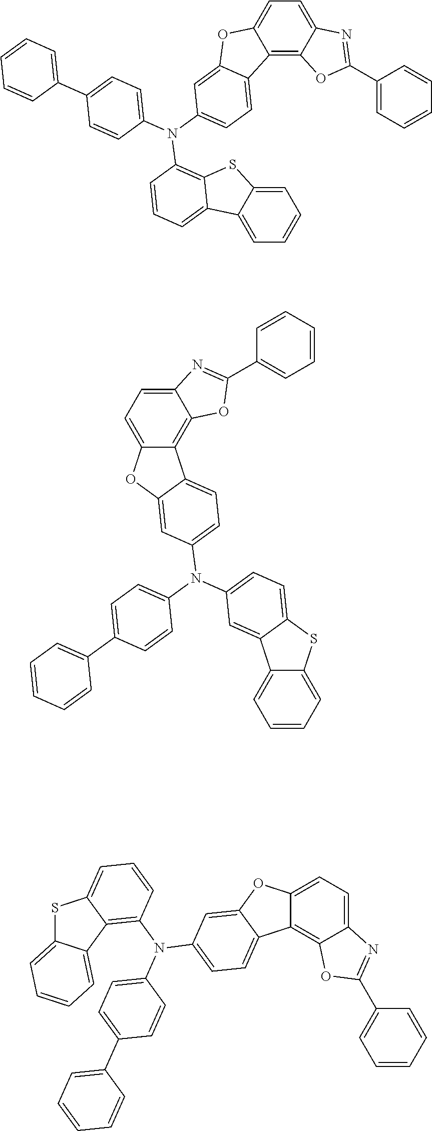 [SMILES: c1ccc(-c2ccc(N(c3ccc4c(c3)oc3ccc5nc(-c6ccccc6)oc5c34)c3cccc4c3sc3ccccc34)cc2)cc1 ; c1ccc(-c2ccc(N(c3ccc4c(c3)oc3ccc5nc(-c6ccccc6)oc5c34)c3ccc4sc5ccccc5c4c3)cc2)cc1 ; c1ccc(-c2ccc(N(c3ccc4c(c3)oc3ccc5nc(-c6ccccc6)oc5c34)c3cccc4sc5ccccc5c34)cc2)cc1]

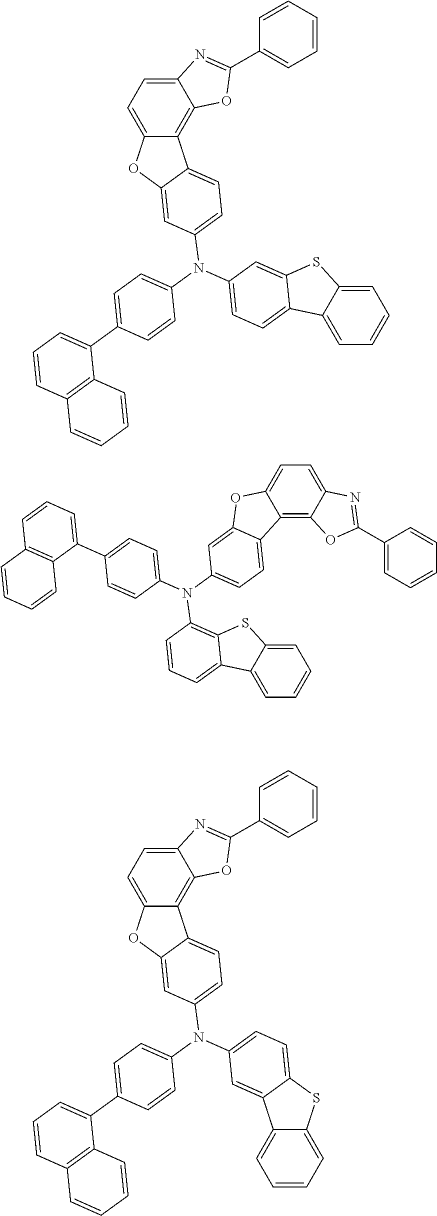 [SMILES: c1ccc(-c2nc3ccc4oc5cc(N(c6ccc(-c7cccc8ccccc78)cc6)c6ccc7c(c6)sc6ccccc67)ccc5c4c3o2)cc1 ; c1ccc(-c2nc3ccc4oc5cc(N(c6ccc(-c7cccc8ccccc78)cc6)c6cccc7c6sc6ccccc67)ccc5c4c3o2)cc1 ; c1ccc(-c2nc3ccc4oc5cc(N(c6ccc(-c7cccc8ccccc78)cc6)c6ccc7sc8ccccc8c7c6)ccc5c4c3o2)cc1]

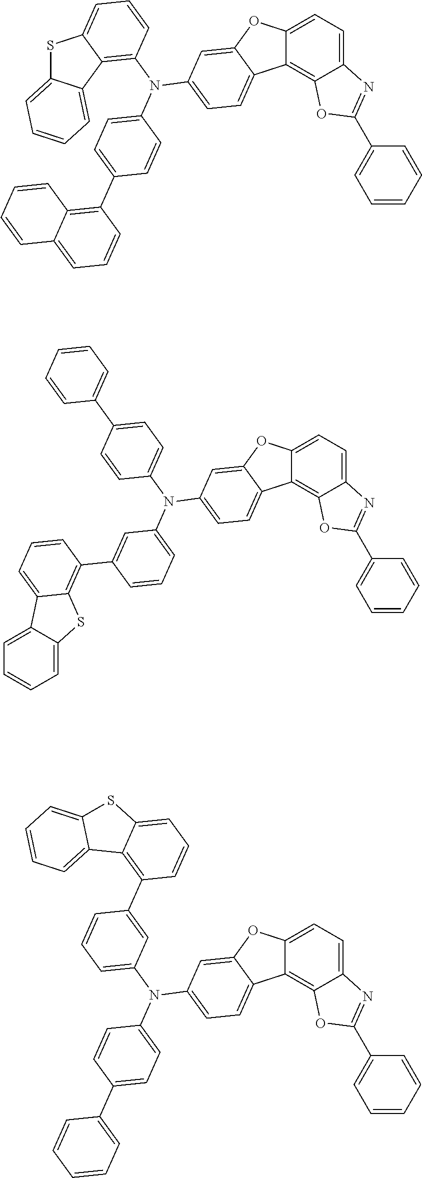 [SMILES: c1ccc(-c2nc3ccc4oc5cc(N(c6ccc(-c7cccc8ccccc78)cc6)c6cccc7sc8ccccc8c67)ccc5c4c3o2)cc1 ; c1ccc(-c2ccc(N(c3cccc(-c4cccc5c4sc4ccccc45)c3)c3ccc4c(c3)oc3ccc5nc(-c6ccccc6)oc5c34)cc2)cc1 ; c1ccc(-c2ccc(N(c3cccc(-c4cccc5sc6ccccc6c45)c3)c3ccc4c(c3)oc3ccc5nc(-c6ccccc6)oc5c34)cc2)cc1]

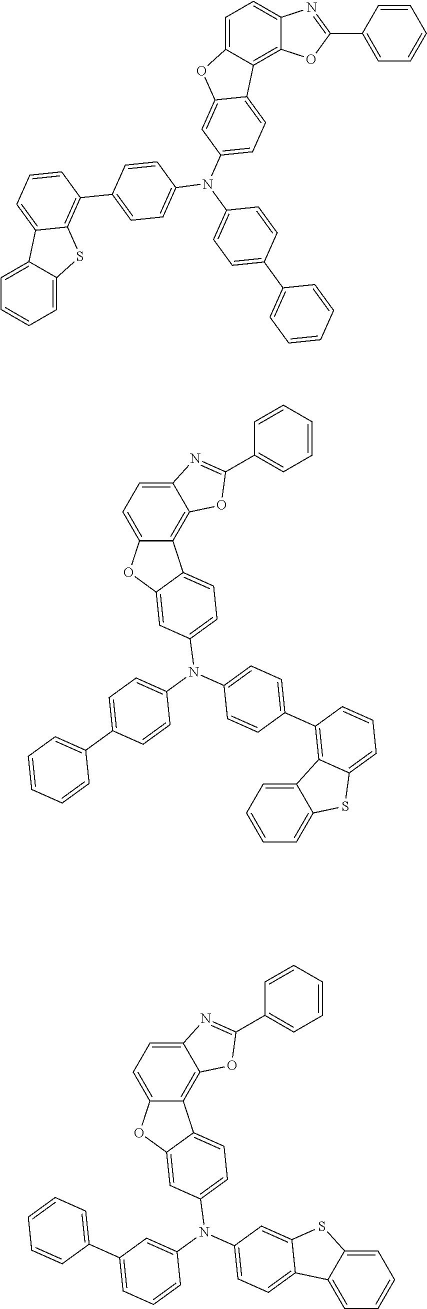 [SMILES: c1ccc(-c2ccc(N(c3ccc(-c4cccc5c4sc4ccccc45)cc3)c3ccc4c(c3)oc3ccc5nc(-c6ccccc6)oc5c34)cc2)cc1 ; c1ccc(-c2ccc(N(c3ccc(-c4cccc5sc6ccccc6c45)cc3)c3ccc4c(c3)oc3ccc5nc(-c6ccccc6)oc5c34)cc2)cc1 ; c1ccc(-c2cccc(N(c3ccc4c(c3)oc3ccc5nc(-c6ccccc6)oc5c34)c3ccc4c(c3)sc3ccccc34)c2)cc1]

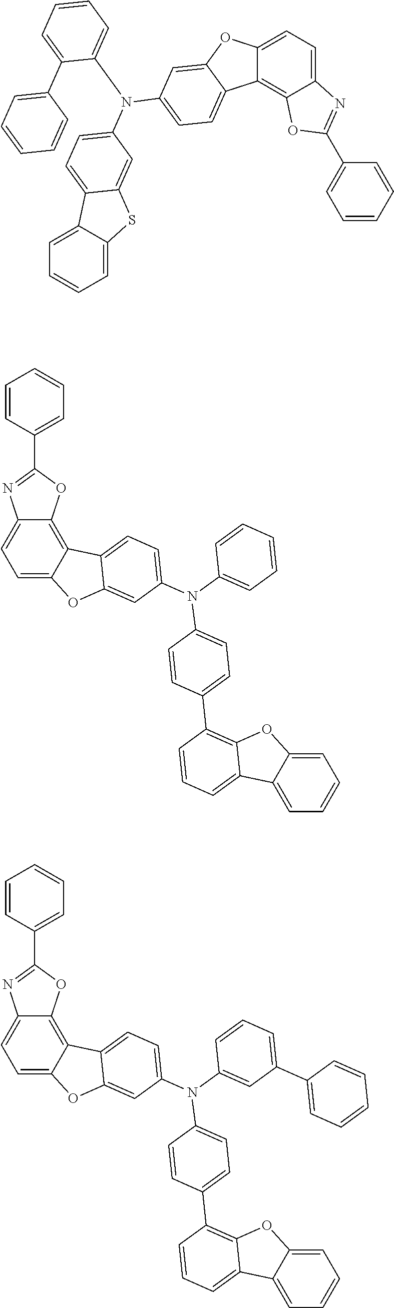 [SMILES: c1ccc(-c2nc3ccc4oc5cc(N(c6ccc7c(c6)sc6ccccc67)c6ccccc6-c6ccccc6)ccc5c4c3o2)cc1 ; c1ccc(-c2nc3ccc4oc5cc(N(c6ccccc6)c6ccc(-c7cccc8c7oc7ccccc78)cc6)ccc5c4c3o2)cc1 ; c1ccc(-c2cccc(N(c3ccc(-c4cccc5c4oc4ccccc45)cc3)c3ccc4c(c3)oc3ccc5nc(-c6ccccc6)oc5c34)c2)cc1]

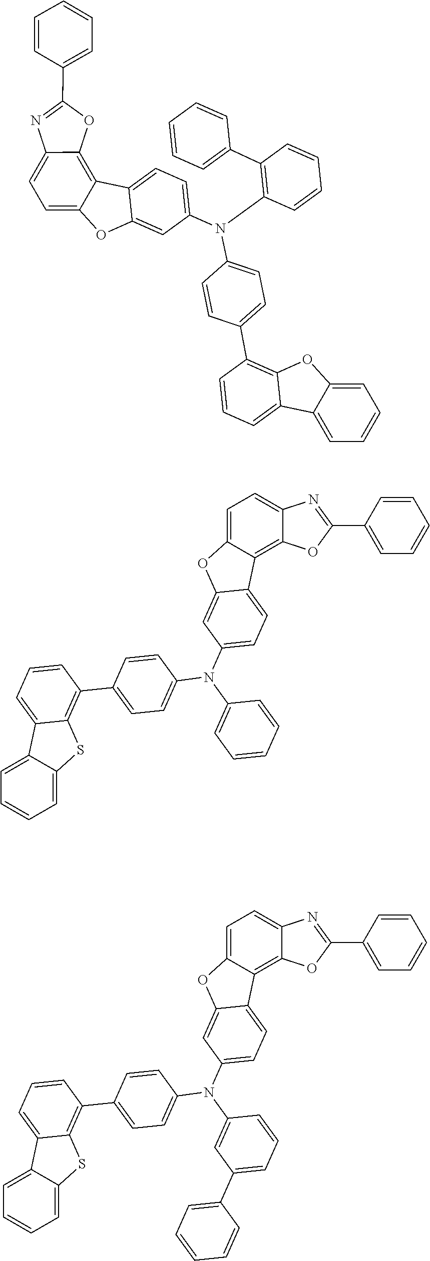 [SMILES: c1ccc(-c2nc3ccc4oc5cc(N(c6ccc(-c7cccc8c7oc7ccccc78)cc6)c6ccccc6-c6ccccc6)ccc5c4c3o2)cc1 ; c1ccc(-c2nc3ccc4oc5cc(N(c6ccccc6)c6ccc(-c7cccc8c7sc7ccccc78)cc6)ccc5c4c3o2)cc1 ; c1ccc(-c2cccc(N(c3ccc(-c4cccc5c4sc4ccccc45)cc3)c3ccc4c(c3)oc3ccc5nc(-c6ccccc6)oc5c34)c2)cc1]

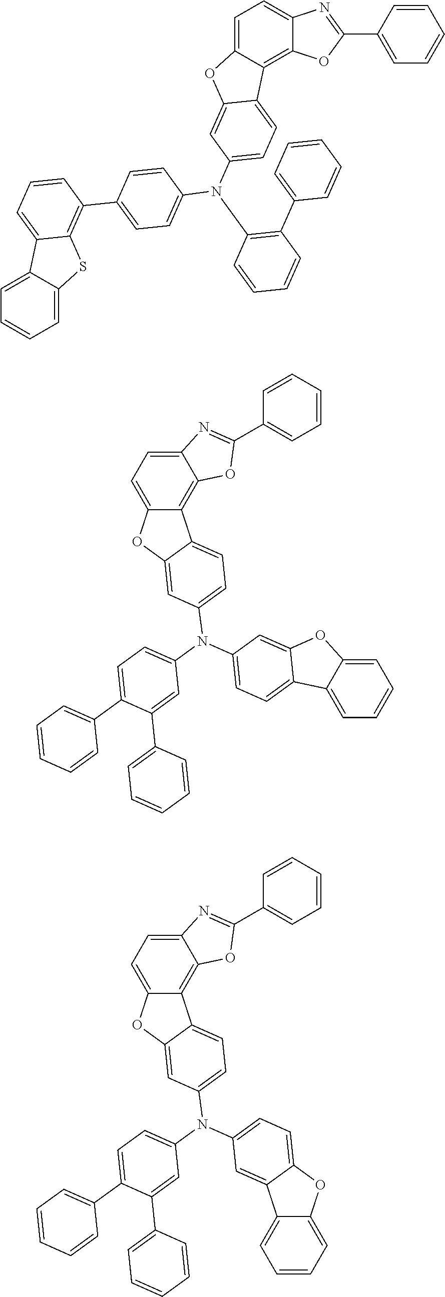 [SMILES: c1ccc(-c2nc3ccc4oc5cc(N(c6ccc(-c7cccc8c7sc7ccccc78)cc6)c6ccccc6-c6ccccc6)ccc5c4c3o2)cc1 ; c1ccc(-c2nc3ccc4oc5cc(N(c6ccc(-c7ccccc7)c(-c7ccccc7)c6)c6ccc7c(c6)oc6ccccc67)ccc5c4c3o2)cc1 ; c1ccc(-c2nc3ccc4oc5cc(N(c6ccc(-c7ccccc7)c(-c7ccccc7)c6)c6ccc7oc8ccccc8c7c6)ccc5c4c3o2)cc1]

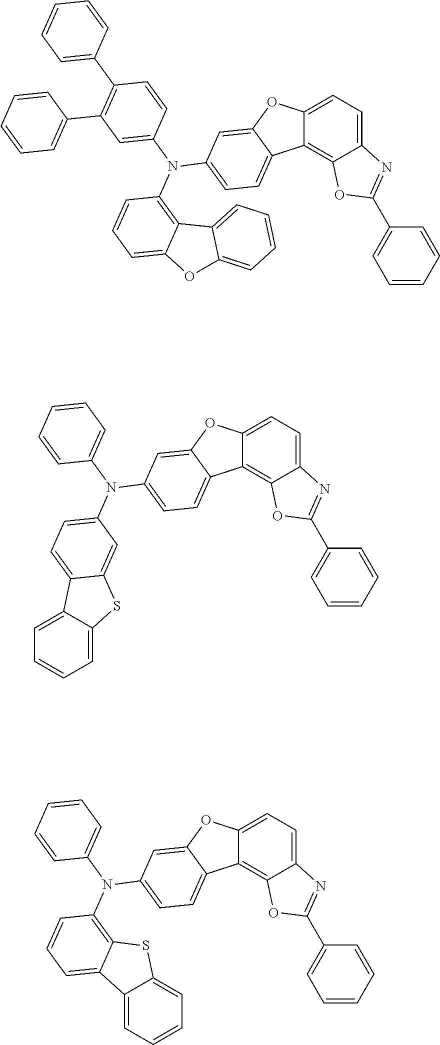 [SMILES: c1ccc(-c2nc3ccc4oc5cc(N(c6ccc(-c7ccccc7)c(-c7ccccc7)c6)c6cccc7oc8ccccc8c67)ccc5c4c3o2)cc1 ; c1ccc(-c2nc3ccc4oc5cc(N(c6ccccc6)c6ccc7c(c6)sc6ccccc67)ccc5c4c3o2)cc1 ; c1ccc(-c2nc3ccc4oc5cc(N(c6ccccc6)c6cccc7c6sc6ccccc67)ccc5c4c3o2)cc1]

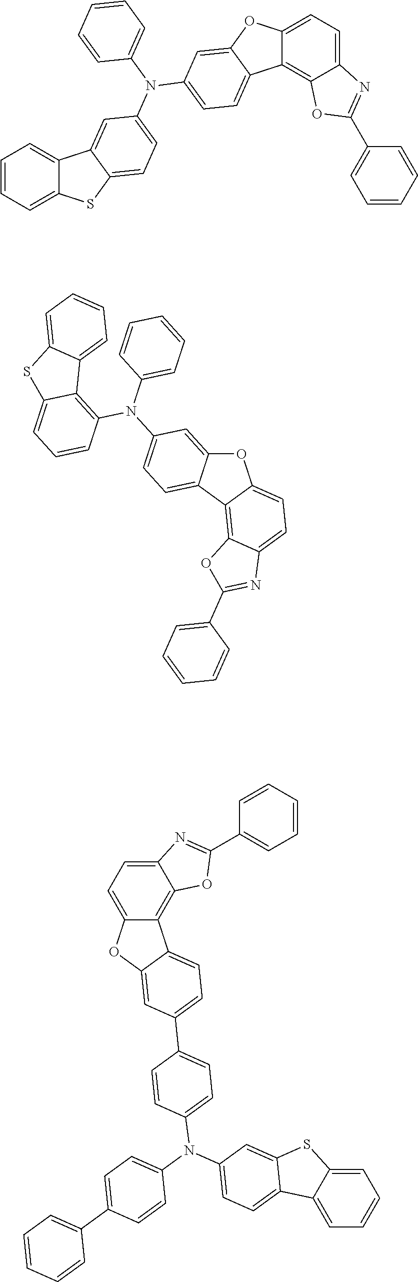 [SMILES: c1ccc(-c2nc3ccc4oc5cc(N(c6ccccc6)c6ccc7sc8ccccc8c7c6)ccc5c4c3o2)cc1 ; c1ccc(-c2nc3ccc4oc5cc(N(c6ccccc6)c6cccc7sc8ccccc8c67)ccc5c4c3o2)cc1 ; c1ccc(-c2ccc(N(c3ccc(-c4ccc5c(c4)oc4ccc6nc(-c7ccccc7)oc6c45)cc3)c3ccc4c(c3)sc3ccccc34)cc2)cc1]

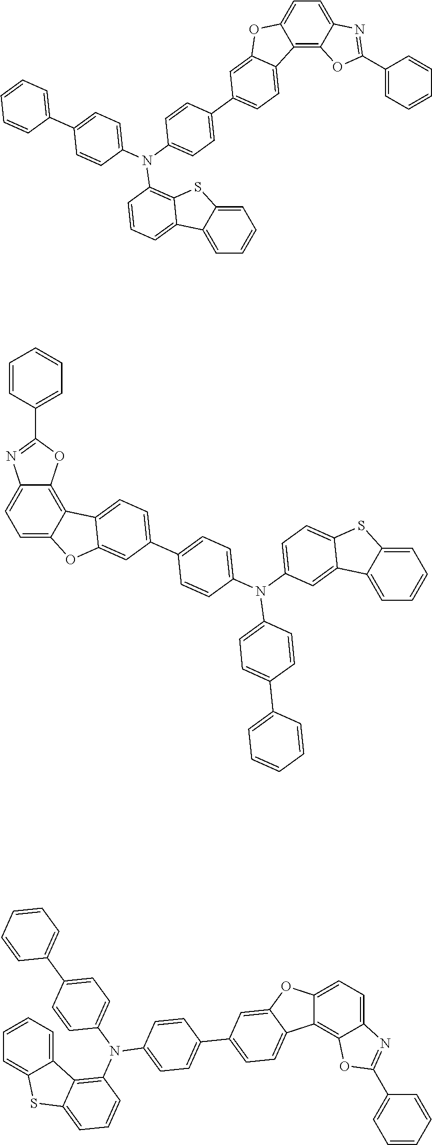 [SMILES: c1ccc(-c2ccc(N(c3ccc(-c4ccc5c(c4)oc4ccc6nc(-c7ccccc7)oc6c45)cc3)c3cccc4c3sc3ccccc34)cc2)cc1 ; c1ccc(-c2ccc(N(c3ccc(-c4ccc5c(c4)oc4ccc6nc(-c7ccccc7)oc6c45)cc3)c3ccc4sc5ccccc5c4c3)cc2)cc1 ; c1ccc(-c2ccc(N(c3ccc(-c4ccc5c(c4)oc4ccc6nc(-c7ccccc7)oc6c45)cc3)c3cccc4sc5ccccc5c34)cc2)cc1]

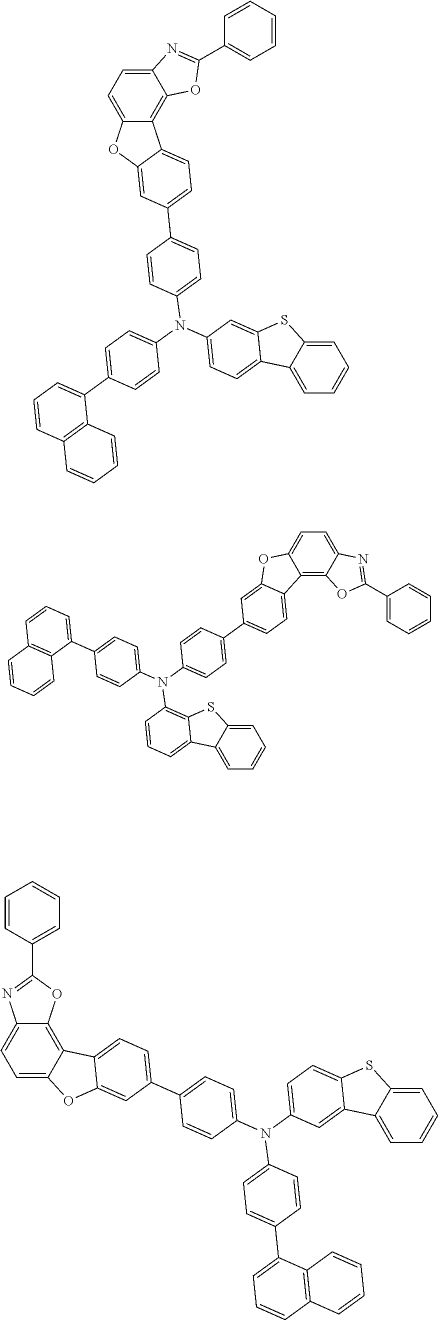 [SMILES: c1ccc(-c2nc3ccc4oc5cc(-c6ccc(N(c7ccc(-c8cccc9ccccc89)cc7)c7ccc8c(c7)sc7ccccc78)cc6)ccc5c4c3o2)cc1 ; c1ccc(-c2nc3ccc4oc5cc(-c6ccc(N(c7ccc(-c8cccc9ccccc89)cc7)c7cccc8c7sc7ccccc78)cc6)ccc5c4c3o2)cc1 ; c1ccc(-c2nc3ccc4oc5cc(-c6ccc(N(c7ccc(-c8cccc9ccccc89)cc7)c7ccc8sc9ccccc9c8c7)cc6)ccc5c4c3o2)cc1]

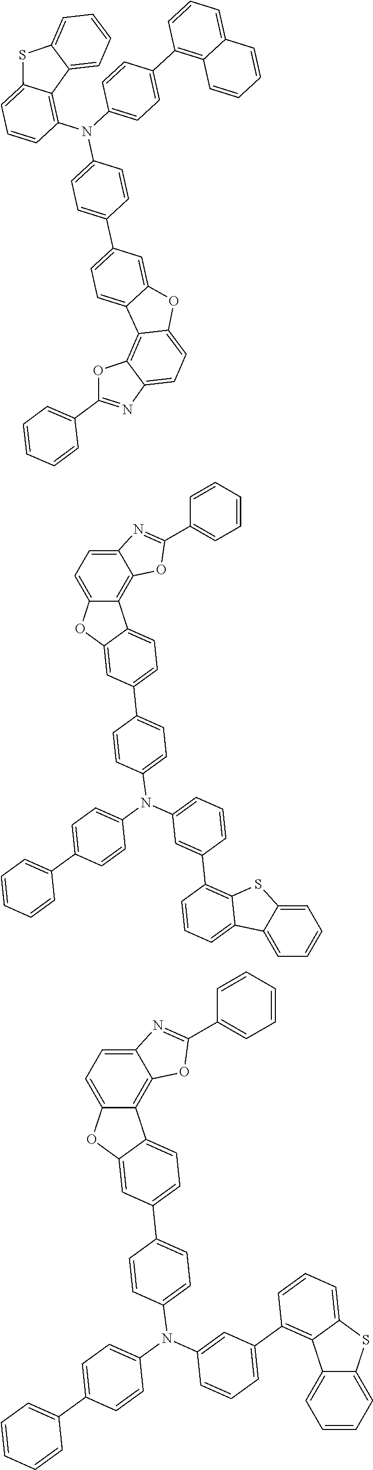

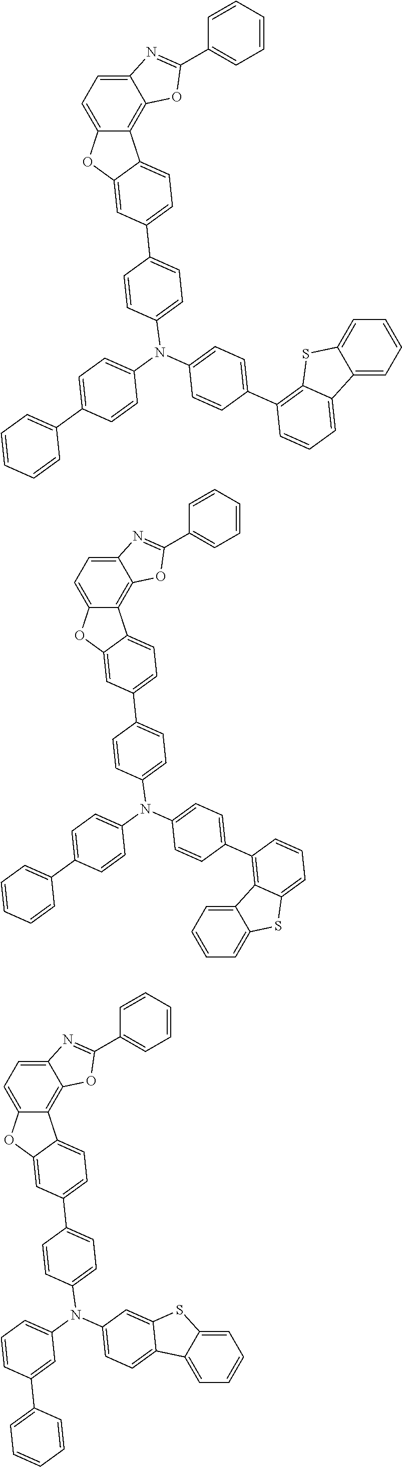 [SMILES: c1ccc(-c2ccc(N(c3ccc(-c4ccc5c(c4)oc4ccc6nc(-c7ccccc7)oc6c45)cc3)c3ccc(-c4cccc5c4sc4ccccc45)cc3)cc2)cc1 ; c1ccc(-c2ccc(N(c3ccc(-c4ccc5c(c4)oc4ccc6nc(-c7ccccc7)oc6c45)cc3)c3ccc(-c4cccc5sc6ccccc6c45)cc3)cc2)cc1 ; c1ccc(-c2cccc(N(c3ccc(-c4ccc5c(c4)oc4ccc6nc(-c7ccccc7)oc6c45)cc3)c3ccc4c(c3)sc3ccccc34)c2)cc1]

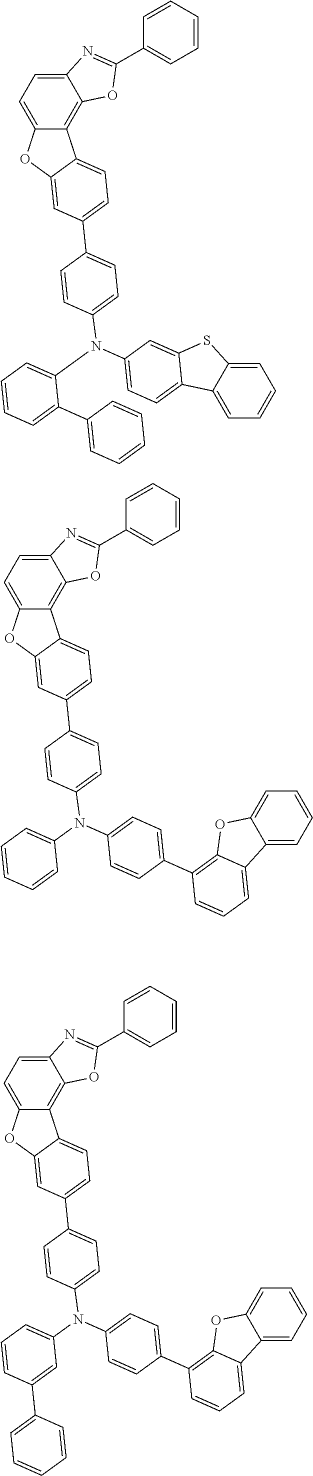

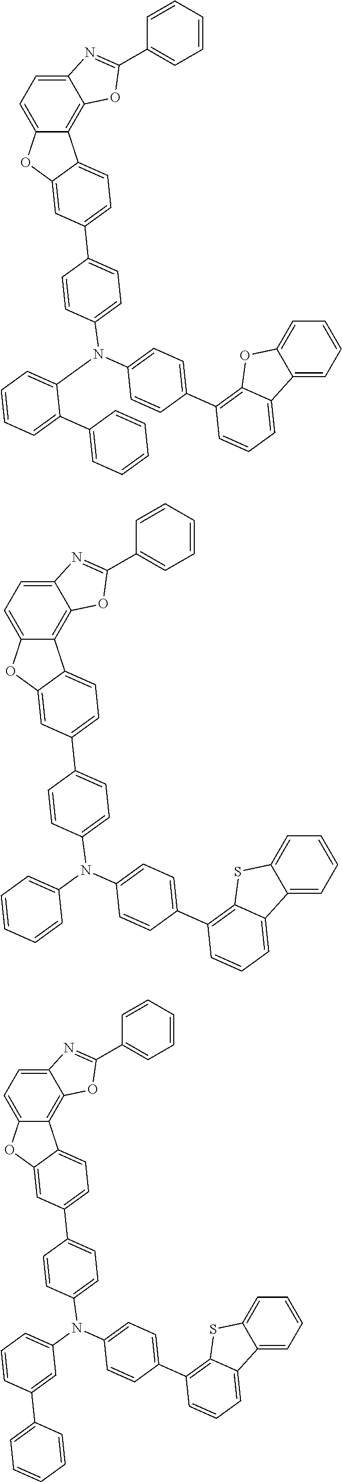 [SMILES: c1ccc(-c2nc3ccc4oc5cc(-c6ccc(N(c7ccc(-c8cccc9c8oc8ccccc89)cc7)c7ccccc7-c7ccccc7)cc6)ccc5c4c3o2)cc1 ; c1ccc(-c2nc3ccc4oc5cc(-c6ccc(N(c7ccccc7)c7ccc(-c8cccc9c8sc8ccccc89)cc7)cc6)ccc5c4c3o2)cc1 ; c1ccc(-c2cccc(N(c3ccc(-c4ccc5c(c4)oc4ccc6nc(-c7ccccc7)oc6c45)cc3)c3ccc(-c4cccc5c4sc4ccccc45)cc3)c2)cc1]

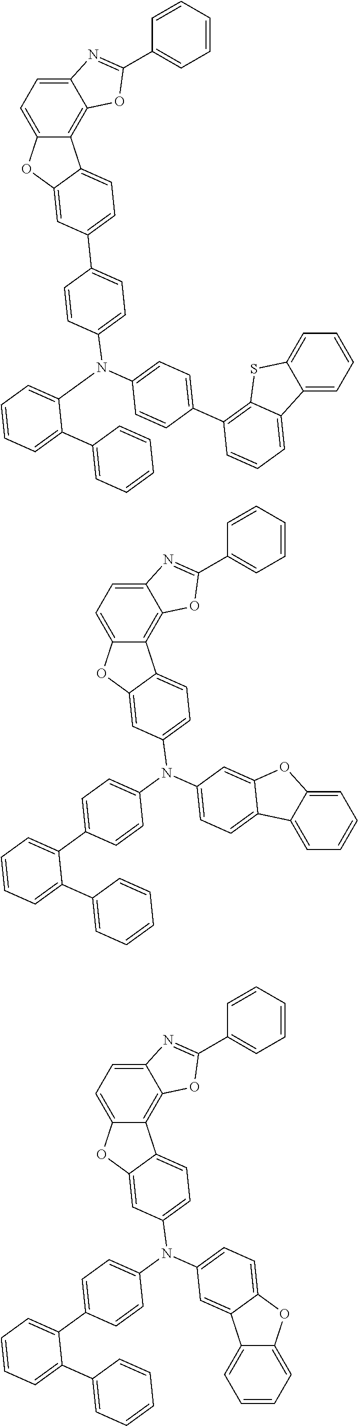

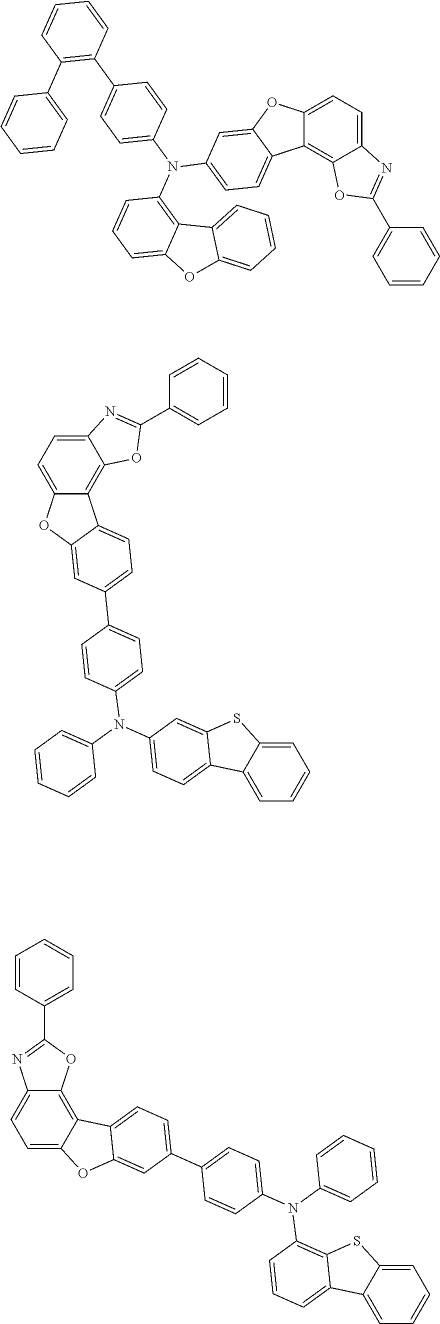 [SMILES: c1ccc(-c2nc3ccc4oc5cc(N(c6ccc(-c7ccccc7-c7ccccc7)cc6)c6cccc7oc8ccccc8c67)ccc5c4c3o2)cc1 ; c1ccc(-c2nc3ccc4oc5cc(-c6ccc(N(c7ccccc7)c7ccc8c(c7)sc7ccccc78)cc6)ccc5c4c3o2)cc1 ; c1ccc(-c2nc3ccc4oc5cc(-c6ccc(N(c7ccccc7)c7cccc8c7sc7ccccc78)cc6)ccc5c4c3o2)cc1]

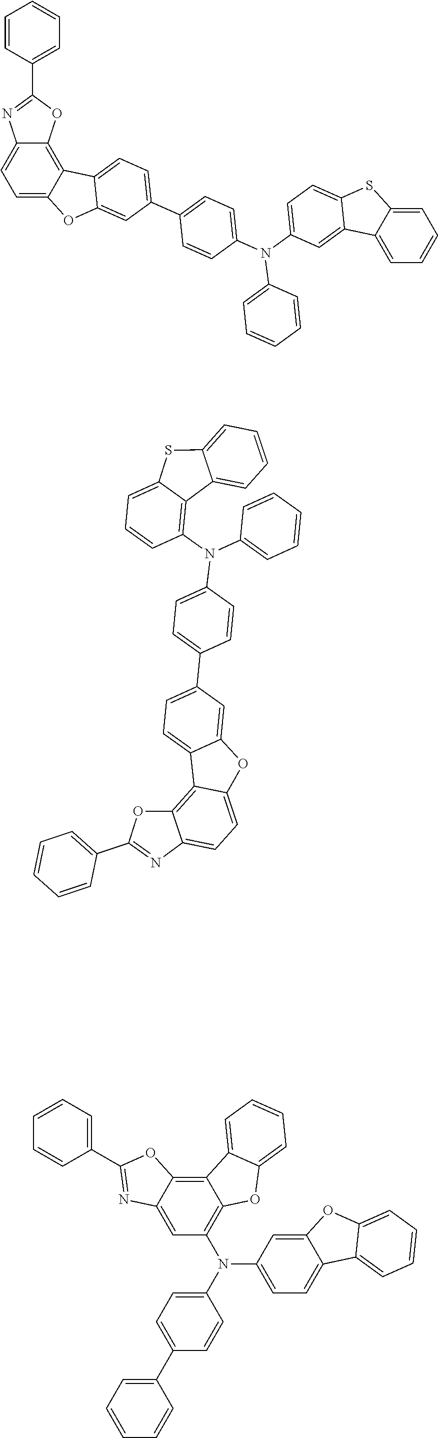 [SMILES: c1ccc(-c2nc3ccc4oc5cc(-c6ccc(N(c7ccccc7)c7ccc8sc9ccccc9c8c7)cc6)ccc5c4c3o2)cc1 ; c1ccc(-c2nc3ccc4oc5cc(-c6ccc(N(c7ccccc7)c7cccc8sc9ccccc9c78)cc6)ccc5c4c3o2)cc1 ; c1ccc(-c2ccc(N(c3ccc4c(c3)oc3ccccc34)c3cc4nc(-c5ccccc5)oc4c4c3oc3ccccc34)cc2)cc1]

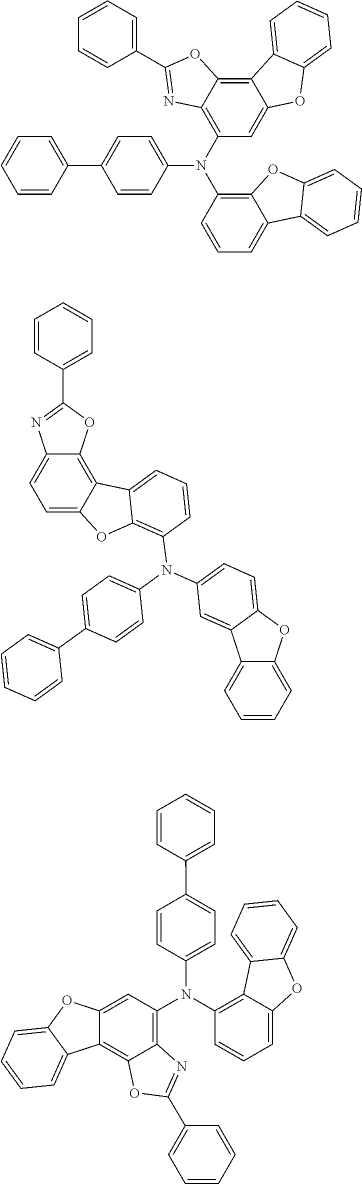 [SMILES: c1ccc(-c2ccc(N(c3cc4oc5ccccc5c4c4oc(-c5ccccc5)nc34)c3cccc4c3oc3ccccc34)cc2)cc1 ; c1ccc(-c2ccc(N(c3ccc4oc5ccccc5c4c3)c3cccc4c3oc3ccc5nc(-c6ccccc6)oc5c34)cc2)cc1 ; c1ccc(-c2ccc(N(c3cc4oc5ccccc5c4c4oc(-c5ccccc5)nc34)c3cccc4oc5ccccc5c34)cc2)cc1]

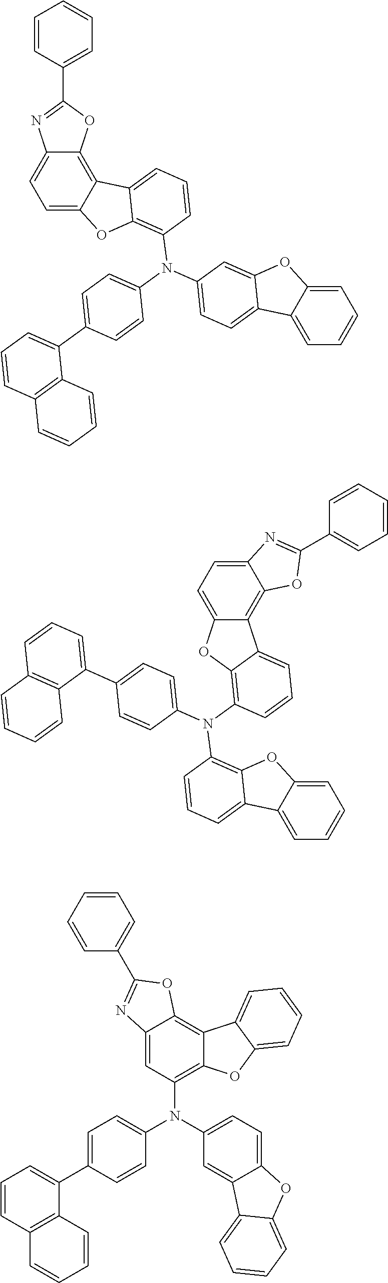 [SMILES: c1ccc(-c2nc3ccc4oc5c(N(c6ccc(-c7cccc8ccccc78)cc6)c6ccc7c(c6)oc6ccccc67)cccc5c4c3o2)cc1 ; c1ccc(-c2nc3ccc4oc5c(N(c6ccc(-c7cccc8ccccc78)cc6)c6cccc7c6oc6ccccc67)cccc5c4c3o2)cc1 ; c1ccc(-c2nc3cc(N(c4ccc(-c5cccc6ccccc56)cc4)c4ccc5oc6ccccc6c5c4)c4oc5ccccc5c4c3o2)cc1]

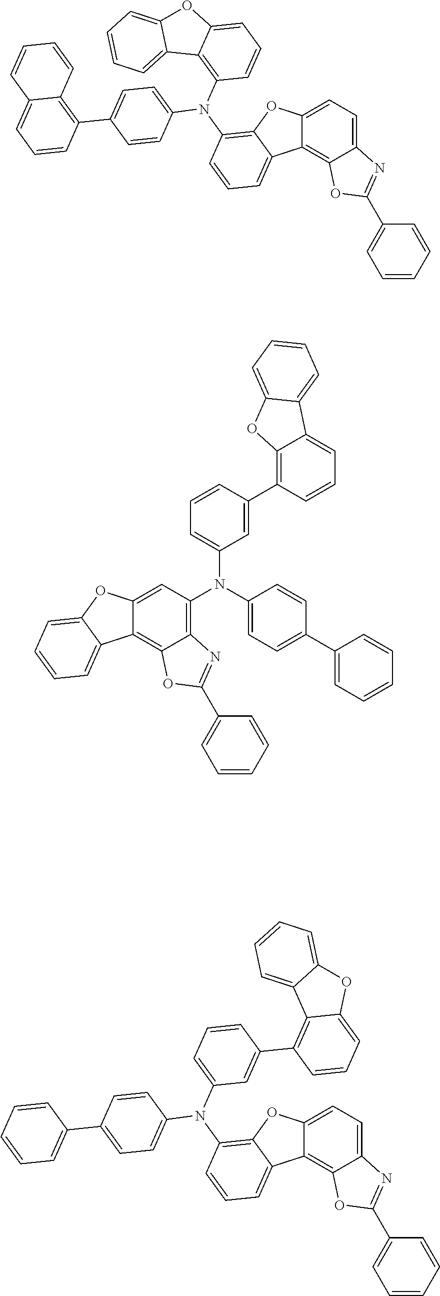 [SMILES: c1ccc(-c2nc3ccc4oc5c(N(c6ccc(-c7cccc8ccccc78)cc6)c6cccc7oc8ccccc8c67)cccc5c4c3o2)cc1 ; c1ccc(-c2ccc(N(c3cccc(-c4cccc5c4oc4ccccc45)c3)c3cc4oc5ccccc5c4c4oc(-c5ccccc5)nc34)cc2)cc1 ; c1ccc(-c2ccc(N(c3cccc(-c4cccc5oc6ccccc6c45)c3)c3cccc4c3oc3ccc5nc(-c6ccccc6)oc5c34)cc2)cc1]

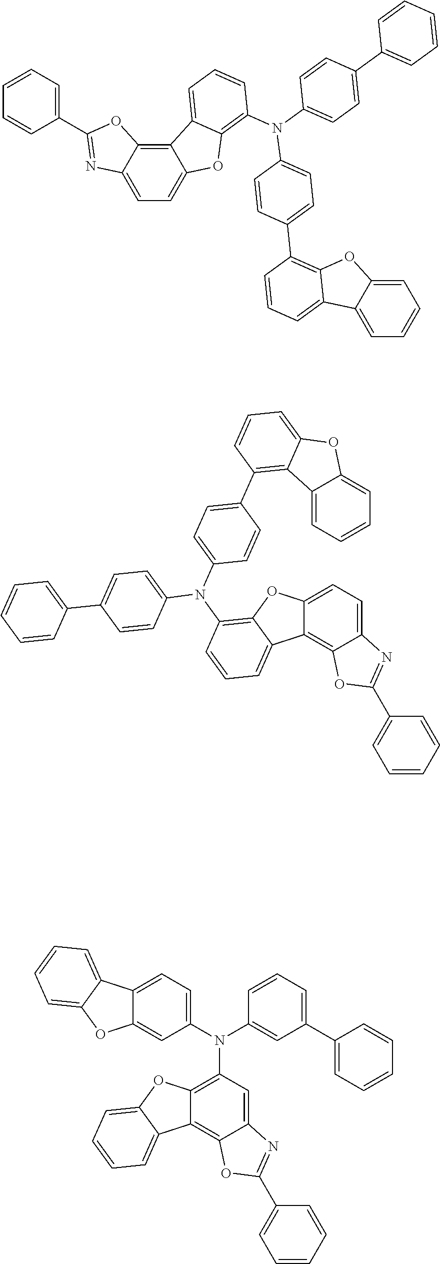 [SMILES: c1ccc(-c2ccc(N(c3ccc(-c4cccc5c4oc4ccccc45)cc3)c3cccc4c3oc3ccc5nc(-c6ccccc6)oc5c34)cc2)cc1 ; c1ccc(-c2ccc(N(c3ccc(-c4cccc5oc6ccccc6c45)cc3)c3cccc4c3oc3ccc5nc(-c6ccccc6)oc5c34)cc2)cc1 ; c1ccc(-c2cccc(N(c3ccc4c(c3)oc3ccccc34)c3cc4nc(-c5ccccc5)oc4c4c3oc3ccccc34)c2)cc1]

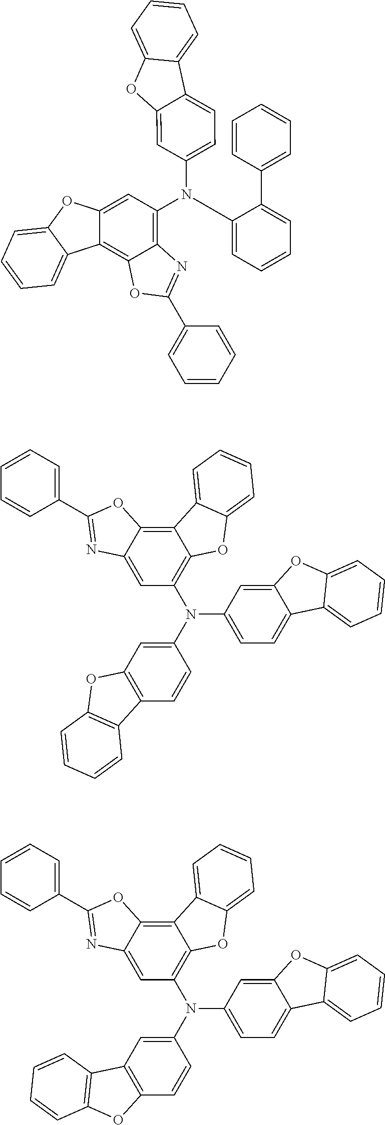 [SMILES: c1ccc(-c2nc3c(N(c4ccc5c(c4)oc4ccccc45)c4ccccc4-c4ccccc4)cc4oc5ccccc5c4c3o2)cc1 ; c1ccc(-c2nc3cc(N(c4ccc5c(c4)oc4ccccc45)c4ccc5c(c4)oc4ccccc45)c4oc5ccccc5c4c3o2)cc1 ; c1ccc(-c2nc3cc(N(c4ccc5c(c4)oc4ccccc45)c4ccc5oc6ccccc6c5c4)c4oc5ccccc5c4c3o2)cc1]

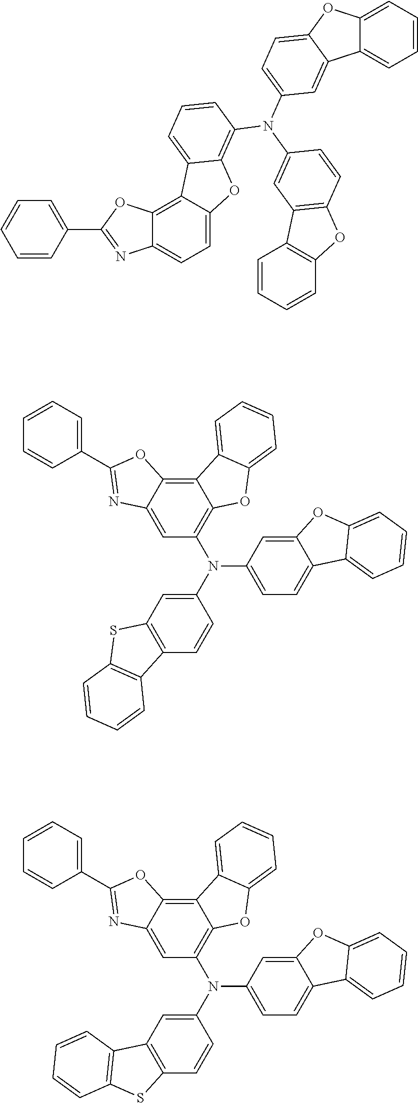 [SMILES: c1ccc(-c2nc3ccc4oc5c(N(c6ccc7oc8ccccc8c7c6)c6ccc7oc8ccccc8c7c6)cccc5c4c3o2)cc1 ; c1ccc(-c2nc3cc(N(c4ccc5c(c4)oc4ccccc45)c4ccc5c(c4)sc4ccccc45)c4oc5ccccc5c4c3o2)cc1 ; c1ccc(-c2nc3cc(N(c4ccc5c(c4)oc4ccccc45)c4ccc5sc6ccccc6c5c4)c4oc5ccccc5c4c3o2)cc1]

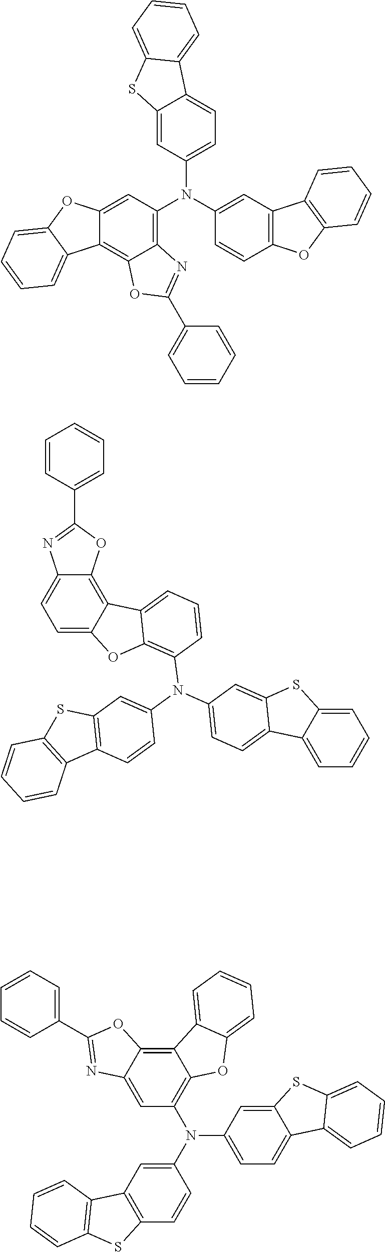 [SMILES: c1ccc(-c2nc3c(N(c4ccc5c(c4)sc4ccccc45)c4ccc5oc6ccccc6c5c4)cc4oc5ccccc5c4c3o2)cc1 ; c1ccc(-c2nc3ccc4oc5c(N(c6ccc7c(c6)sc6ccccc67)c6ccc7c(c6)sc6ccccc67)cccc5c4c3o2)cc1 ; c1ccc(-c2nc3cc(N(c4ccc5c(c4)sc4ccccc45)c4ccc5sc6ccccc6c5c4)c4oc5ccccc5c4c3o2)cc1]

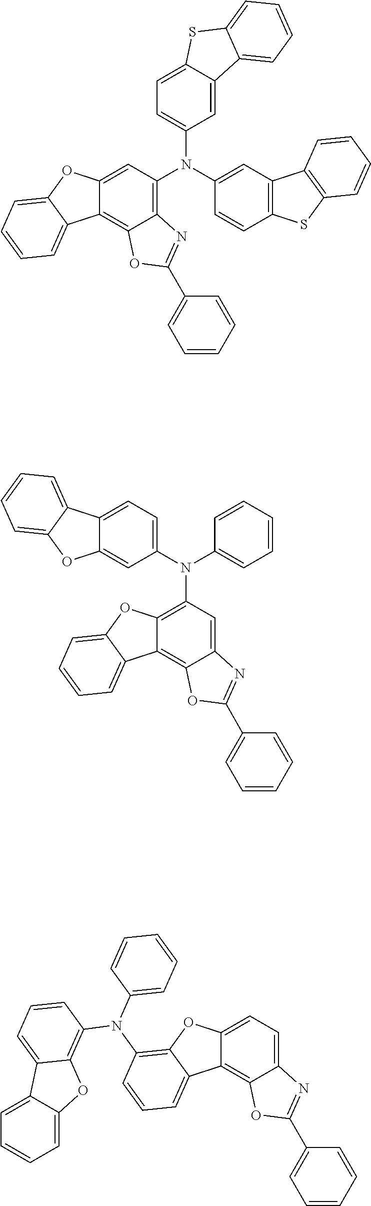 [SMILES: c1ccc(-c2nc3c(N(c4ccc5sc6ccccc6c5c4)c4ccc5sc6ccccc6c5c4)cc4oc5ccccc5c4c3o2)cc1 ; c1ccc(-c2nc3cc(N(c4ccccc4)c4ccc5c(c4)oc4ccccc45)c4oc5ccccc5c4c3o2)cc1 ; c1ccc(-c2nc3ccc4oc5c(N(c6ccccc6)c6cccc7c6oc6ccccc67)cccc5c4c3o2)cc1]

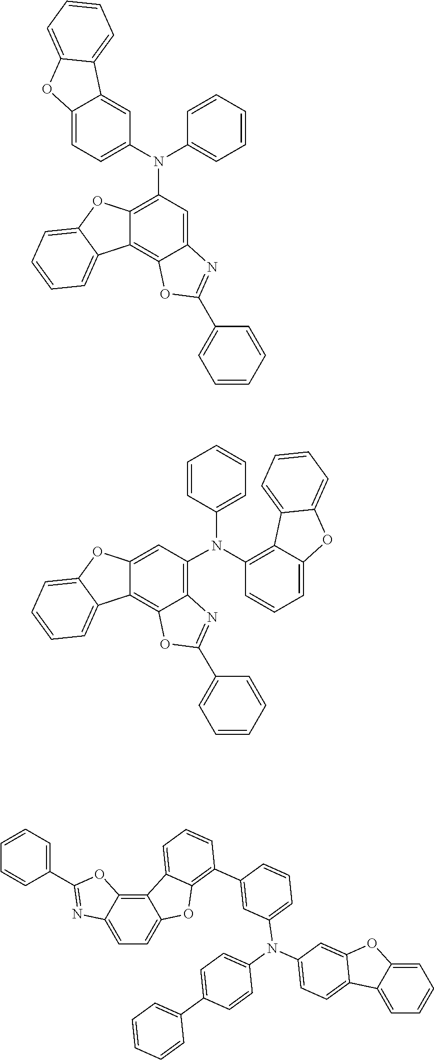 [SMILES: c1ccc(-c2nc3cc(N(c4ccccc4)c4ccc5oc6ccccc6c5c4)c4oc5ccccc5c4c3o2)cc1 ; c1ccc(-c2nc3c(N(c4ccccc4)c4cccc5oc6ccccc6c45)cc4oc5ccccc5c4c3o2)cc1 ; c1ccc(-c2ccc(N(c3cccc(-c4cccc5c4oc4ccc6nc(-c7ccccc7)oc6c45)c3)c3ccc4c(c3)oc3ccccc34)cc2)cc1]

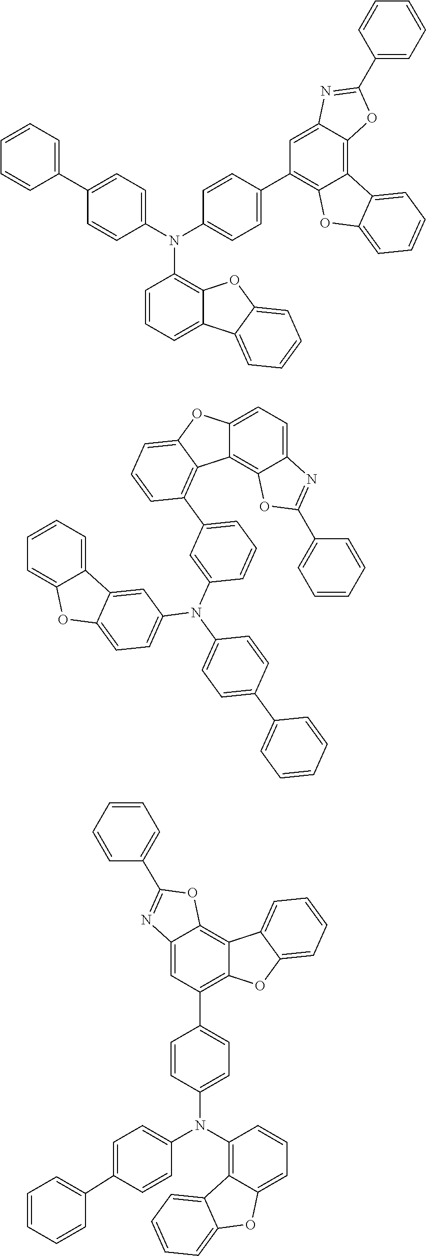

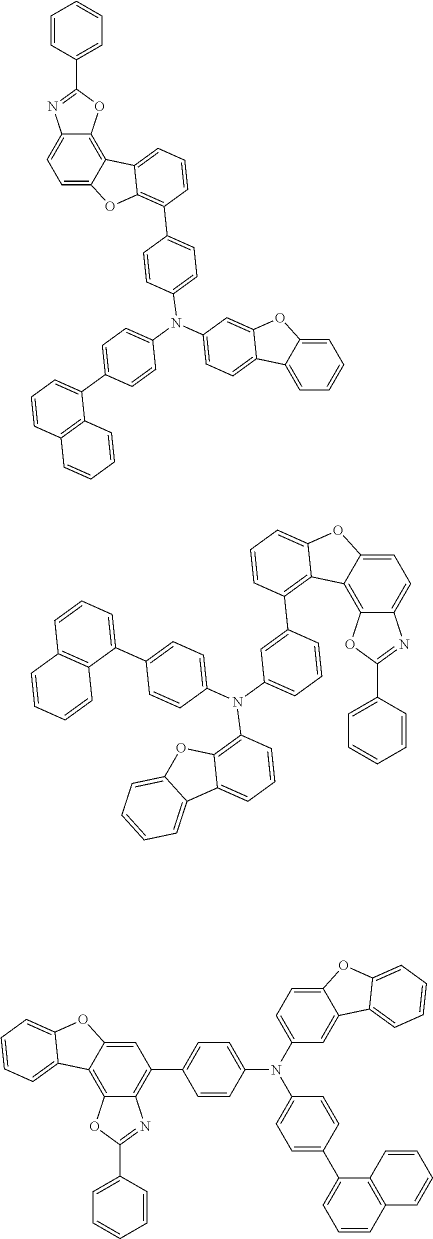 [SMILES: c1ccc(-c2nc3ccc4oc5c(-c6ccc(N(c7ccc(-c8cccc9ccccc89)cc7)c7ccc8c(c7)oc7ccccc78)cc6)cccc5c4c3o2)cc1 ; c1ccc(-c2nc3ccc4oc5cccc(-c6cccc(N(c7ccc(-c8cccc9ccccc89)cc7)c7cccc8c7oc7ccccc78)c6)c5c4c3o2)cc1 ; c1ccc(-c2nc3c(-c4ccc(N(c5ccc(-c6cccc7ccccc67)cc5)c5ccc6oc7ccccc7c6c5)cc4)cc4oc5ccccc5c4c3o2)cc1]

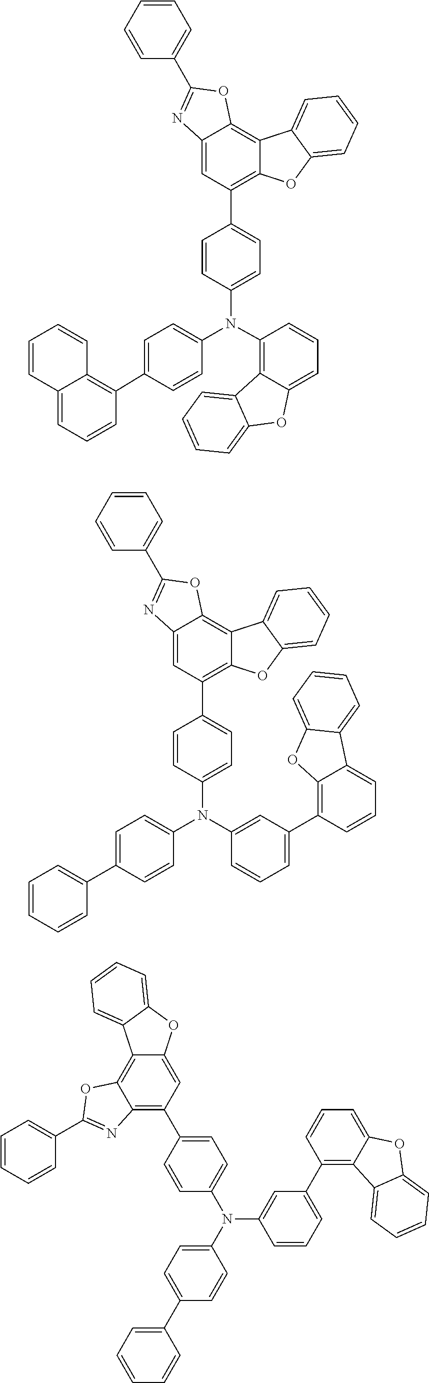 [SMILES: c1ccc(-c2nc3cc(-c4ccc(N(c5ccc(-c6cccc7ccccc67)cc5)c5cccc6oc7ccccc7c56)cc4)c4oc5ccccc5c4c3o2)cc1 ; c1ccc(-c2ccc(N(c3ccc(-c4cc5nc(-c6ccccc6)oc5c5c4oc4ccccc45)cc3)c3cccc(-c4cccc5c4oc4ccccc45)c3)cc2)cc1 ; c1ccc(-c2ccc(N(c3ccc(-c4cc5oc6ccccc6c5c5oc(-c6ccccc6)nc45)cc3)c3cccc(-c4cccc5oc6ccccc6c45)c3)cc2)cc1]

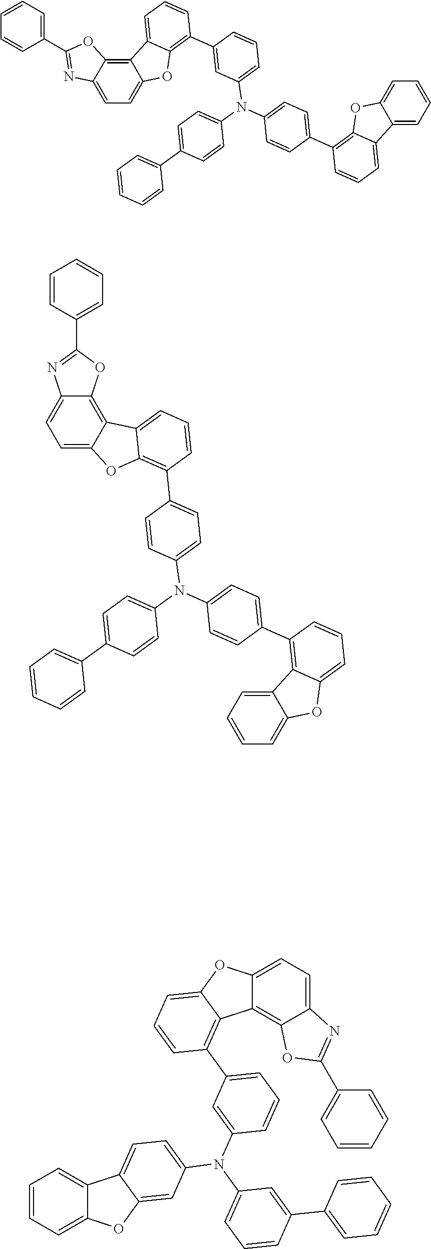 [SMILES: c1ccc(-c2ccc(N(c3ccc(-c4cccc5c4oc4ccccc45)cc3)c3cccc(-c4cccc5c4oc4ccc6nc(-c7ccccc7)oc6c45)c3)cc2)cc1 ; c1ccc(-c2ccc(N(c3ccc(-c4cccc5c4oc4ccc6nc(-c7ccccc7)oc6c45)cc3)c3ccc(-c4cccc5oc6ccccc6c45)cc3)cc2)cc1 ; c1ccc(-c2cccc(N(c3cccc(-c4cccc5oc6ccc7nc(-c8ccccc8)oc7c6c45)c3)c3ccc4c(c3)oc3ccccc34)c2)cc1]

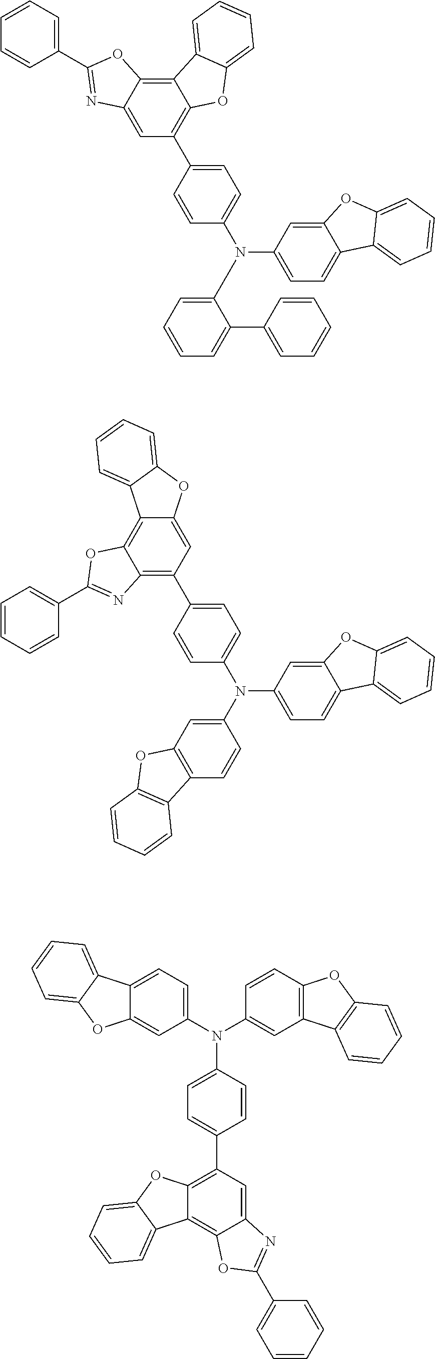 [SMILES: c1ccc(-c2nc3cc(-c4ccc(N(c5ccc6c(c5)oc5ccccc56)c5ccccc5-c5ccccc5)cc4)c4oc5ccccc5c4c3o2)cc1 ; c1ccc(-c2nc3c(-c4ccc(N(c5ccc6c(c5)oc5ccccc56)c5ccc6c(c5)oc5ccccc56)cc4)cc4oc5ccccc5c4c3o2)cc1 ; c1ccc(-c2nc3cc(-c4ccc(N(c5ccc6c(c5)oc5ccccc56)c5ccc6oc7ccccc7c6c5)cc4)c4oc5ccccc5c4c3o2)cc1]

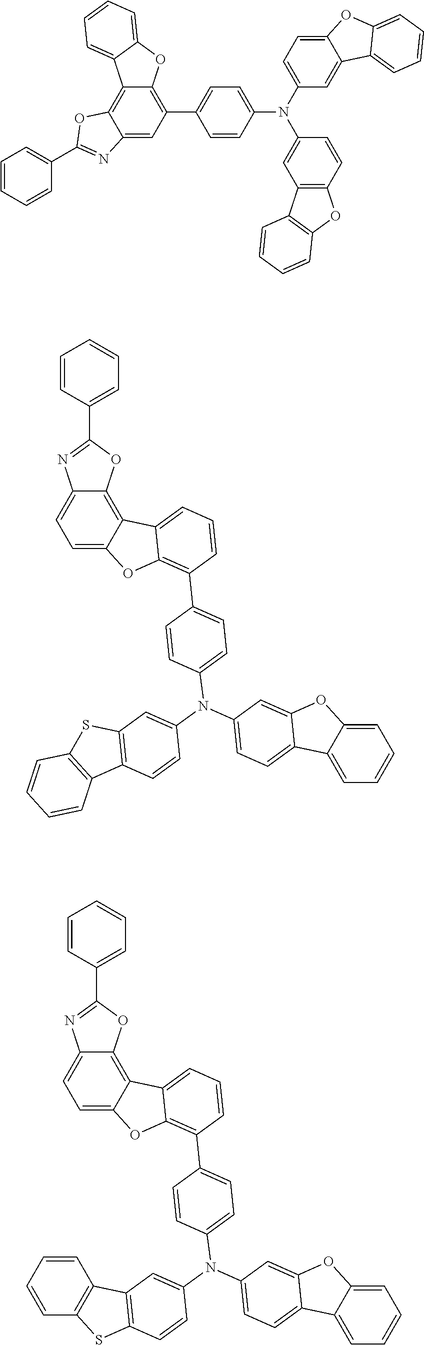 [SMILES: c1ccc(-c2nc3cc(-c4ccc(N(c5ccc6oc7ccccc7c6c5)c5ccc6oc7ccccc7c6c5)cc4)c4oc5ccccc5c4c3o2)cc1 ; c1ccc(-c2nc3ccc4oc5c(-c6ccc(N(c7ccc8c(c7)oc7ccccc78)c7ccc8c(c7)sc7ccccc78)cc6)cccc5c4c3o2)cc1 ; c1ccc(-c2nc3ccc4oc5c(-c6ccc(N(c7ccc8c(c7)oc7ccccc78)c7ccc8sc9ccccc9c8c7)cc6)cccc5c4c3o2)cc1]

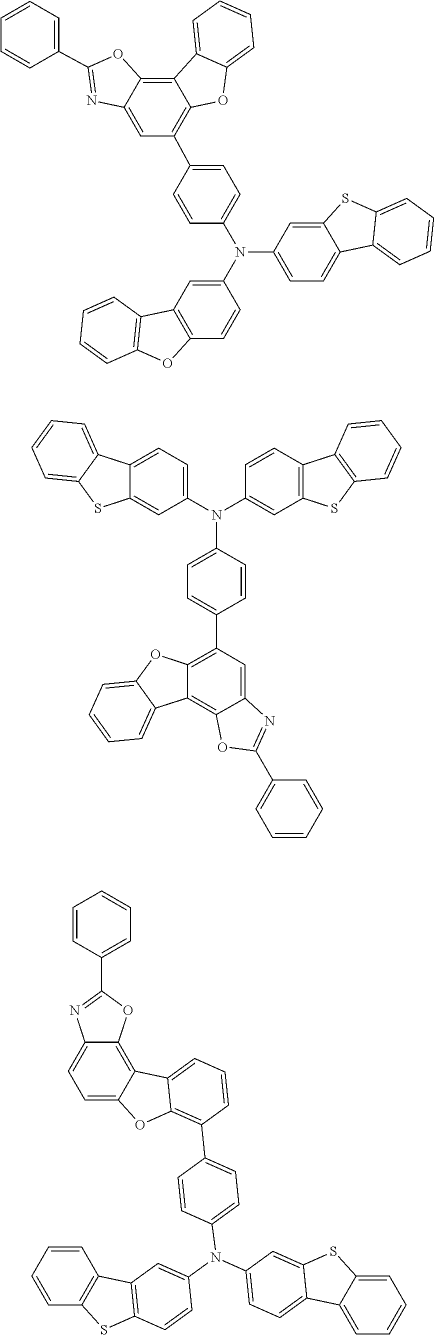 [SMILES: c1ccc(-c2nc3cc(-c4ccc(N(c5ccc6c(c5)sc5ccccc56)c5ccc6oc7ccccc7c6c5)cc4)c4oc5ccccc5c4c3o2)cc1 ; c1ccc(-c2nc3cc(-c4ccc(N(c5ccc6c(c5)sc5ccccc56)c5ccc6c(c5)sc5ccccc56)cc4)c4oc5ccccc5c4c3o2)cc1 ; c1ccc(-c2nc3ccc4oc5c(-c6ccc(N(c7ccc8c(c7)sc7ccccc78)c7ccc8sc9ccccc9c8c7)cc6)cccc5c4c3o2)cc1]

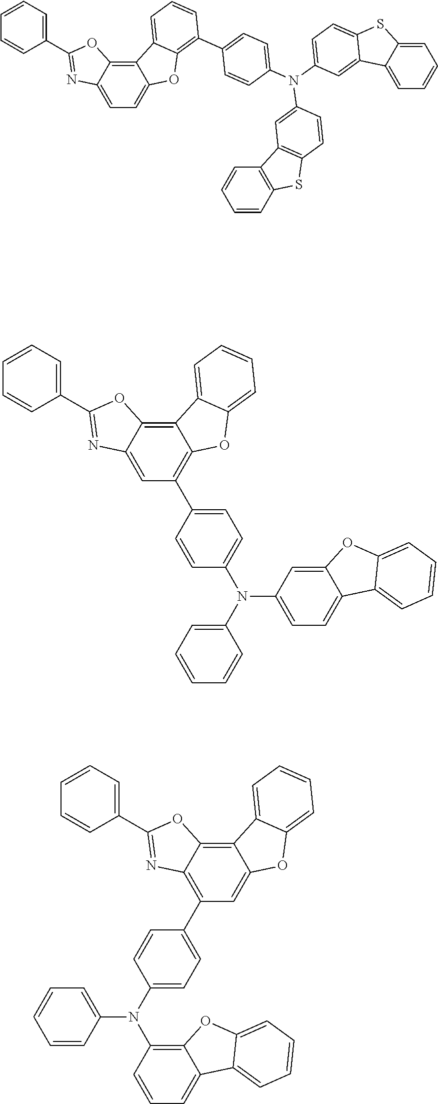 [SMILES: c1ccc(-c2nc3ccc4oc5c(-c6ccc(N(c7ccc8sc9ccccc9c8c7)c7ccc8sc9ccccc9c8c7)cc6)cccc5c4c3o2)cc1 ; c1ccc(-c2nc3cc(-c4ccc(N(c5ccccc5)c5ccc6c(c5)oc5ccccc56)cc4)c4oc5ccccc5c4c3o2)cc1 ; c1ccc(-c2nc3c(-c4ccc(N(c5ccccc5)c5cccc6c5oc5ccccc56)cc4)cc4oc5ccccc5c4c3o2)cc1]

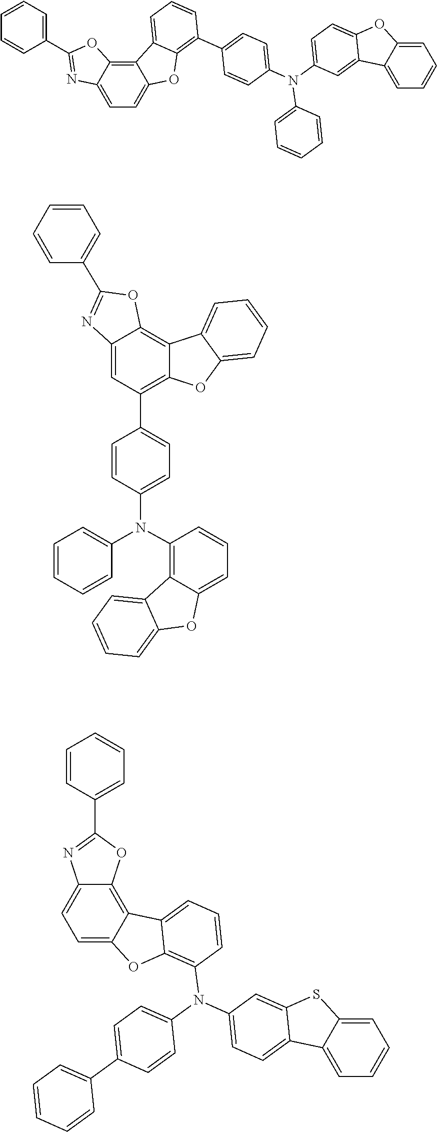 [SMILES: c1ccc(-c2nc3ccc4oc5c(-c6ccc(N(c7ccccc7)c7ccc8oc9ccccc9c8c7)cc6)cccc5c4c3o2)cc1 ; c1ccc(-c2nc3cc(-c4ccc(N(c5ccccc5)c5cccc6oc7ccccc7c56)cc4)c4oc5ccccc5c4c3o2)cc1 ; c1ccc(-c2ccc(N(c3ccc4c(c3)sc3ccccc34)c3cccc4c3oc3ccc5nc(-c6ccccc6)oc5c34)cc2)cc1]

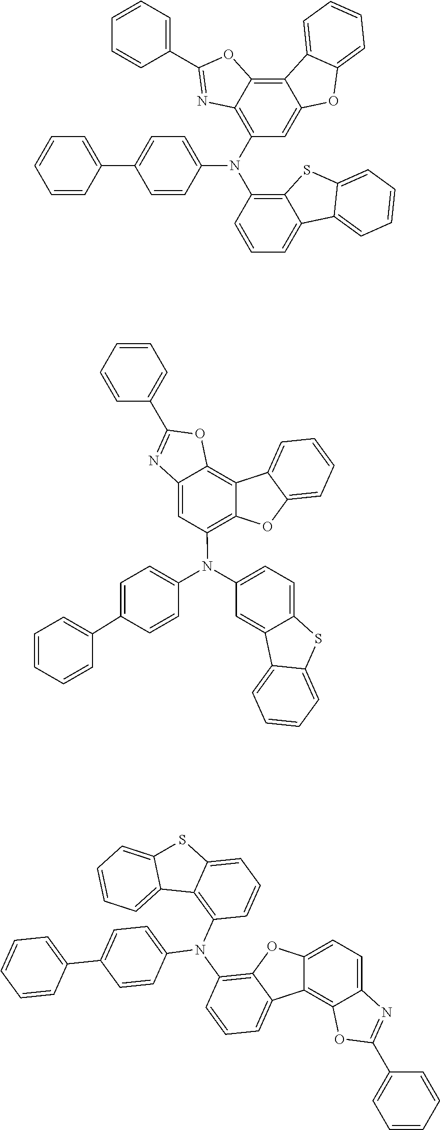 [SMILES: c1ccc(-c2ccc(N(c3cc4oc5ccccc5c4c4oc(-c5ccccc5)nc34)c3cccc4c3sc3ccccc34)cc2)cc1 ; c1ccc(-c2ccc(N(c3ccc4sc5ccccc5c4c3)c3cc4nc(-c5ccccc5)oc4c4c3oc3ccccc34)cc2)cc1 ; c1ccc(-c2ccc(N(c3cccc4c3oc3ccc5nc(-c6ccccc6)oc5c34)c3cccc4sc5ccccc5c34)cc2)cc1]

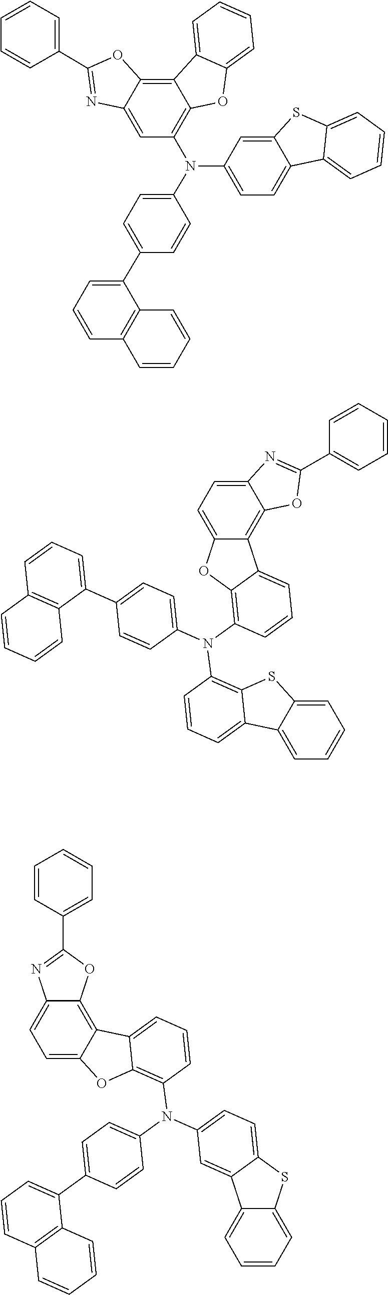 [SMILES: c1ccc(-c2nc3cc(N(c4ccc(-c5cccc6ccccc56)cc4)c4ccc5c(c4)sc4ccccc45)c4oc5ccccc5c4c3o2)cc1 ; c1ccc(-c2nc3ccc4oc5c(N(c6ccc(-c7cccc8ccccc78)cc6)c6cccc7c6sc6ccccc67)cccc5c4c3o2)cc1 ; c1ccc(-c2nc3ccc4oc5c(N(c6ccc(-c7cccc8ccccc78)cc6)c6ccc7sc8ccccc8c7c6)cccc5c4c3o2)cc1]

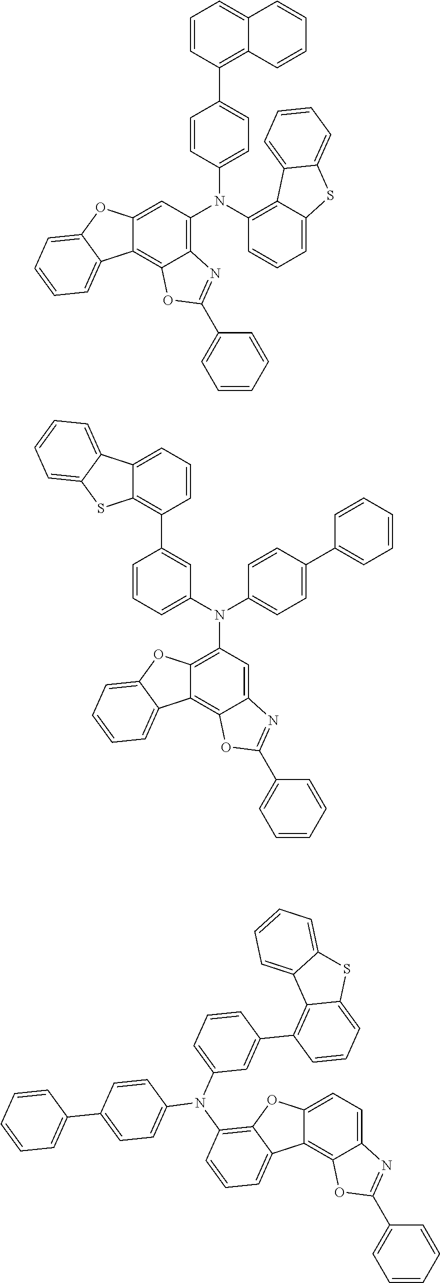 [SMILES: c1ccc(-c2nc3c(N(c4ccc(-c5cccc6ccccc56)cc4)c4cccc5sc6ccccc6c45)cc4oc5ccccc5c4c3o2)cc1 ; c1ccc(-c2ccc(N(c3cccc(-c4cccc5c4sc4ccccc45)c3)c3cc4nc(-c5ccccc5)oc4c4c3oc3ccccc34)cc2)cc1 ; c1ccc(-c2ccc(N(c3cccc(-c4cccc5sc6ccccc6c45)c3)c3cccc4c3oc3ccc5nc(-c6ccccc6)oc5c34)cc2)cc1]

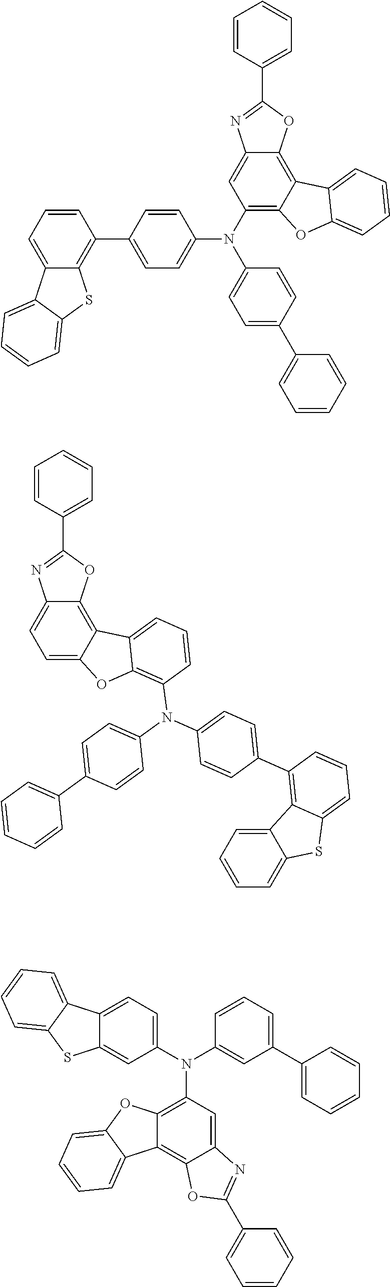 [SMILES: c1ccc(-c2ccc(N(c3ccc(-c4cccc5c4sc4ccccc45)cc3)c3cc4nc(-c5ccccc5)oc4c4c3oc3ccccc34)cc2)cc1 ; c1ccc(-c2ccc(N(c3ccc(-c4cccc5sc6ccccc6c45)cc3)c3cccc4c3oc3ccc5nc(-c6ccccc6)oc5c34)cc2)cc1 ; c1ccc(-c2cccc(N(c3ccc4c(c3)sc3ccccc34)c3cc4nc(-c5ccccc5)oc4c4c3oc3ccccc34)c2)cc1]

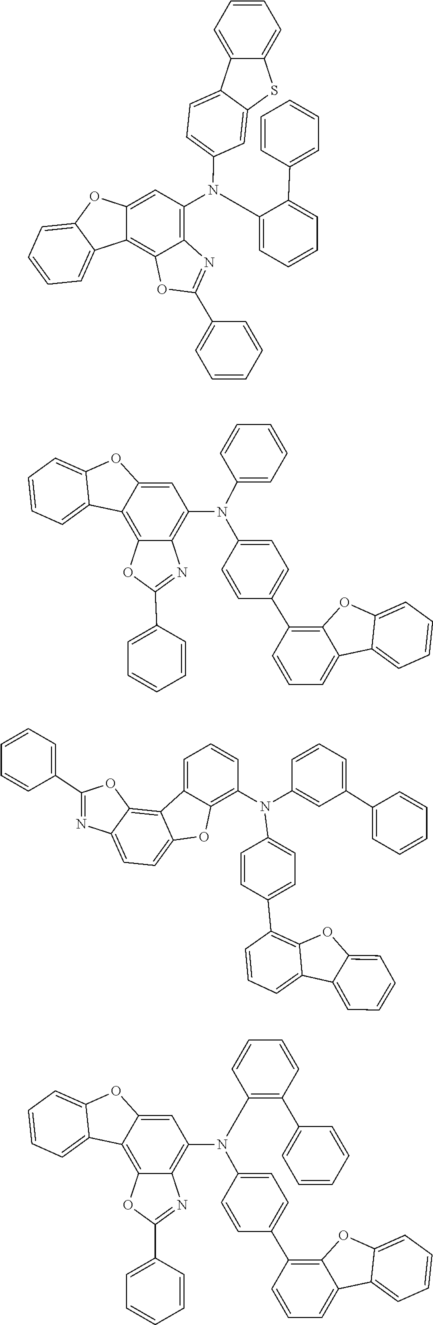

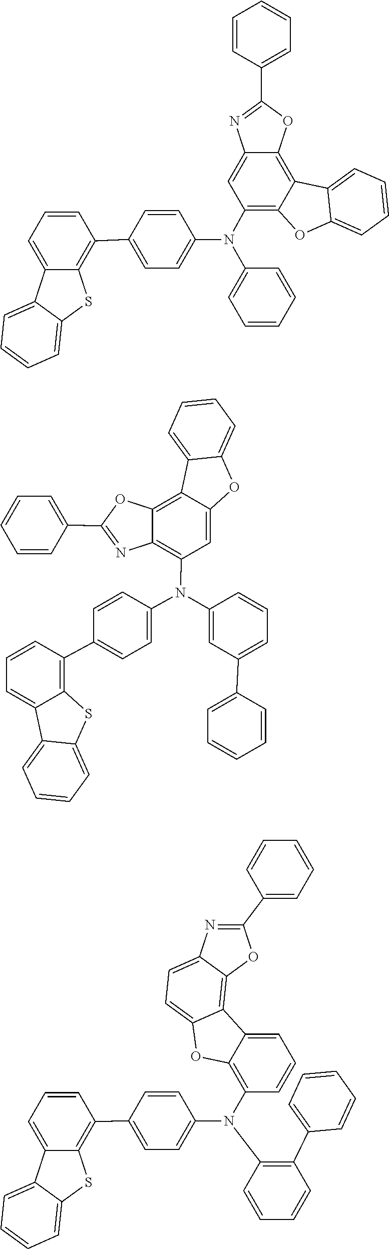 [SMILES: c1ccc(-c2nc3cc(N(c4ccccc4)c4ccc(-c5cccc6c5sc5ccccc56)cc4)c4oc5ccccc5c4c3o2)cc1 ; c1ccc(-c2cccc(N(c3ccc(-c4cccc5c4sc4ccccc45)cc3)c3cc4oc5ccccc5c4c4oc(-c5ccccc5)nc34)c2)cc1 ; c1ccc(-c2nc3ccc4oc5c(N(c6ccc(-c7cccc8c7sc7ccccc78)cc6)c6ccccc6-c6ccccc6)cccc5c4c3o2)cc1]

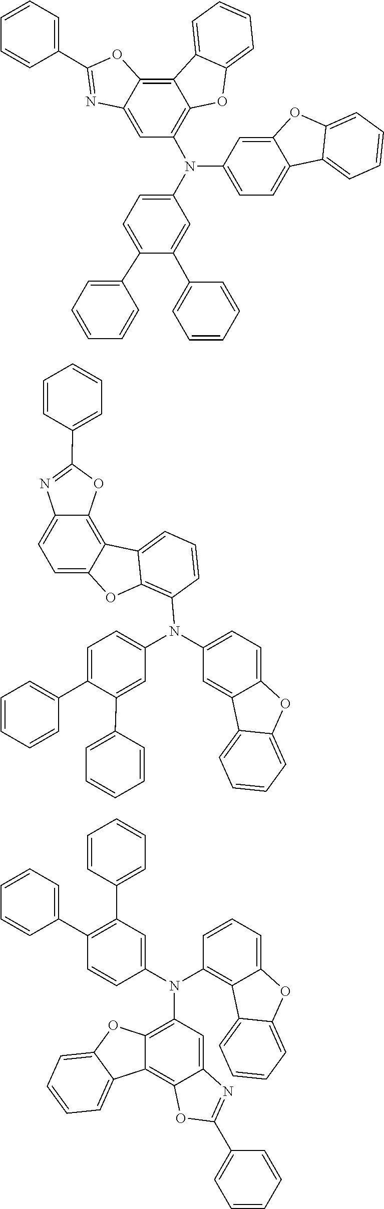

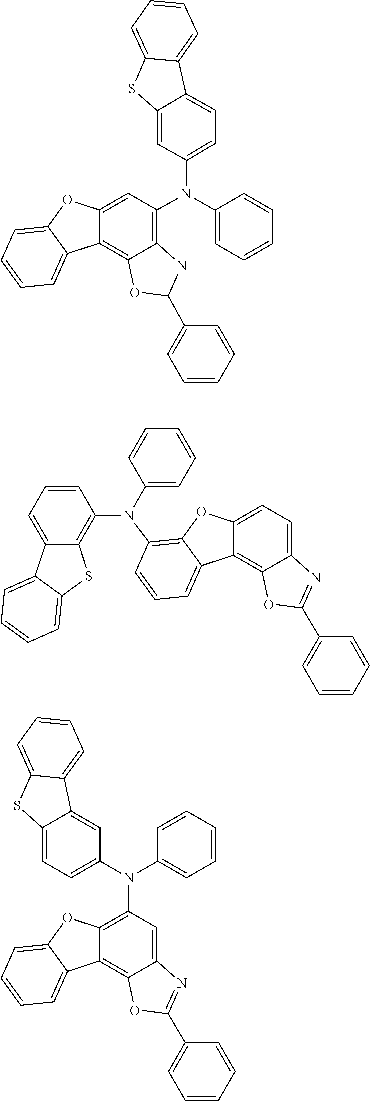

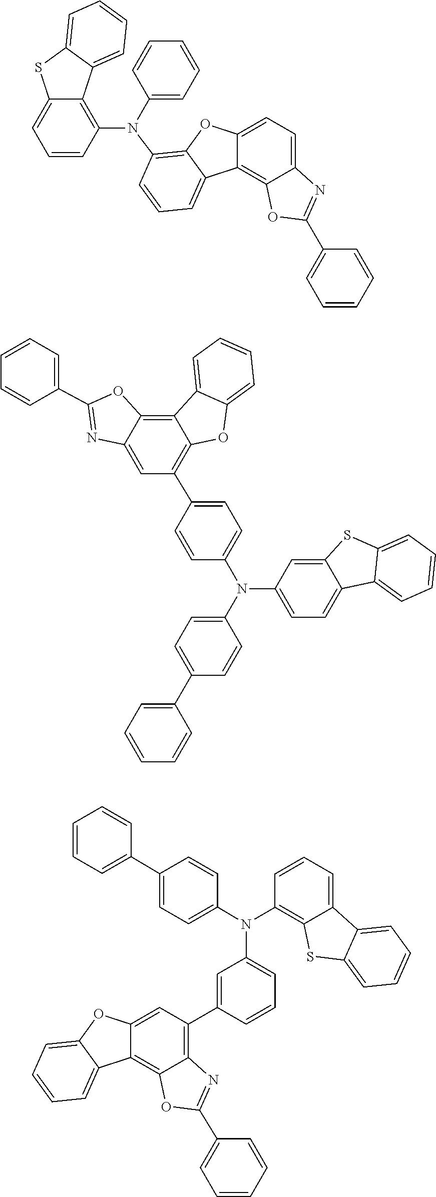 [SMILES: c1ccc(-c2nc3ccc4oc5c(N(c6ccccc6)c6cccc7sc8ccccc8c67)cccc5c4c3o2)cc1 ; c1ccc(-c2ccc(N(c3ccc(-c4cc5nc(-c6ccccc6)oc5c5c4oc4ccccc45)cc3)c3ccc4c(c3)sc3ccccc34)cc2)cc1 ; c1ccc(-c2ccc(N(c3cccc(-c4cc5oc6ccccc6c5c5oc(-c6ccccc6)nc45)c3)c3cccc4c3sc3ccccc34)cc2)cc1]

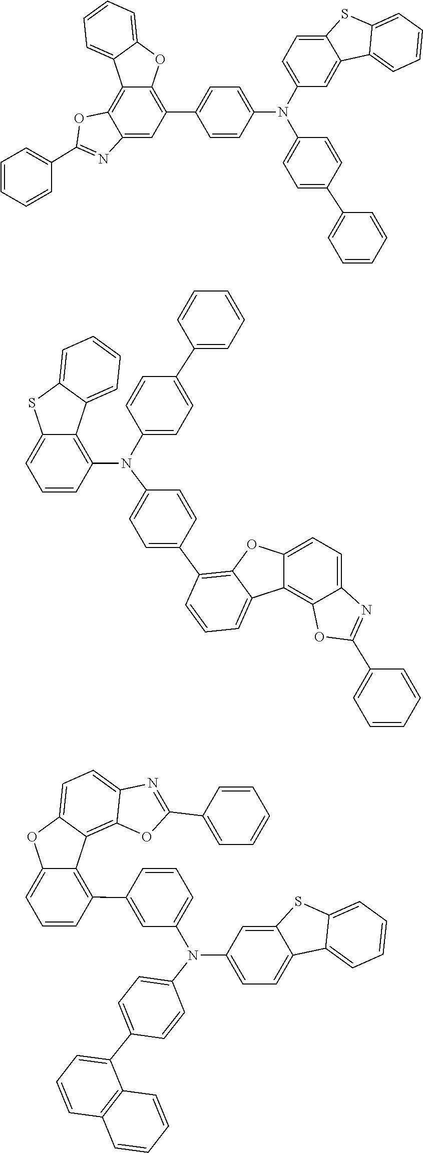 [SMILES: c1ccc(-c2ccc(N(c3ccc(-c4cc5nc(-c6ccccc6)oc5c5c4oc4ccccc45)cc3)c3ccc4sc5ccccc5c4c3)cc2)cc1 ; c1ccc(-c2ccc(N(c3ccc(-c4cccc5c4oc4ccc6nc(-c7ccccc7)oc6c45)cc3)c3cccc4sc5ccccc5c34)cc2)cc1 ; c1ccc(-c2nc3ccc4oc5cccc(-c6cccc(N(c7ccc(-c8cccc9ccccc89)cc7)c7ccc8c(c7)sc7ccccc78)c6)c5c4c3o2)cc1]

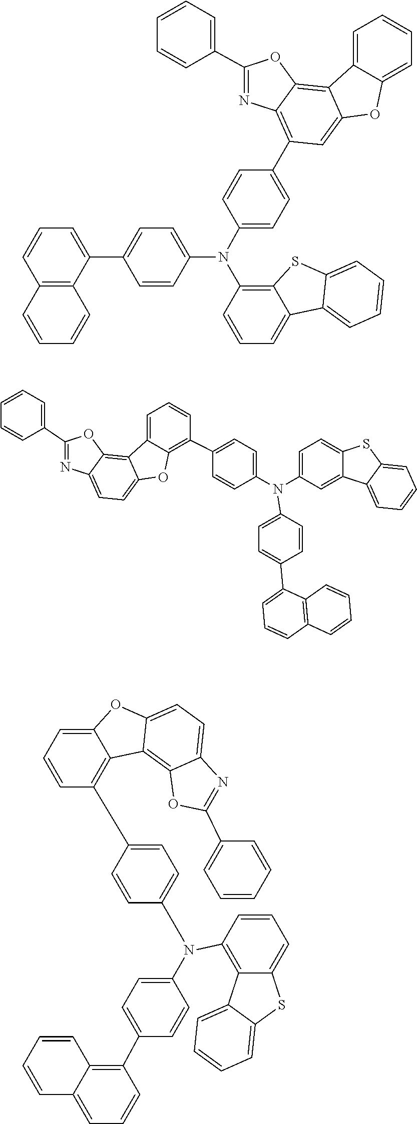 [SMILES: c1ccc(-c2nc3c(-c4ccc(N(c5ccc(-c6cccc7ccccc67)cc5)c5cccc6c5sc5ccccc56)cc4)cc4oc5ccccc5c4c3o2)cc1 ; c1ccc(-c2nc3ccc4oc5c(-c6ccc(N(c7ccc(-c8cccc9ccccc89)cc7)c7ccc8sc9ccccc9c8c7)cc6)cccc5c4c3o2)cc1 ; c1ccc(-c2nc3ccc4oc5cccc(-c6ccc(N(c7ccc(-c8cccc9ccccc89)cc7)c7cccc8sc9ccccc9c78)cc6)c5c4c3o2)cc1]

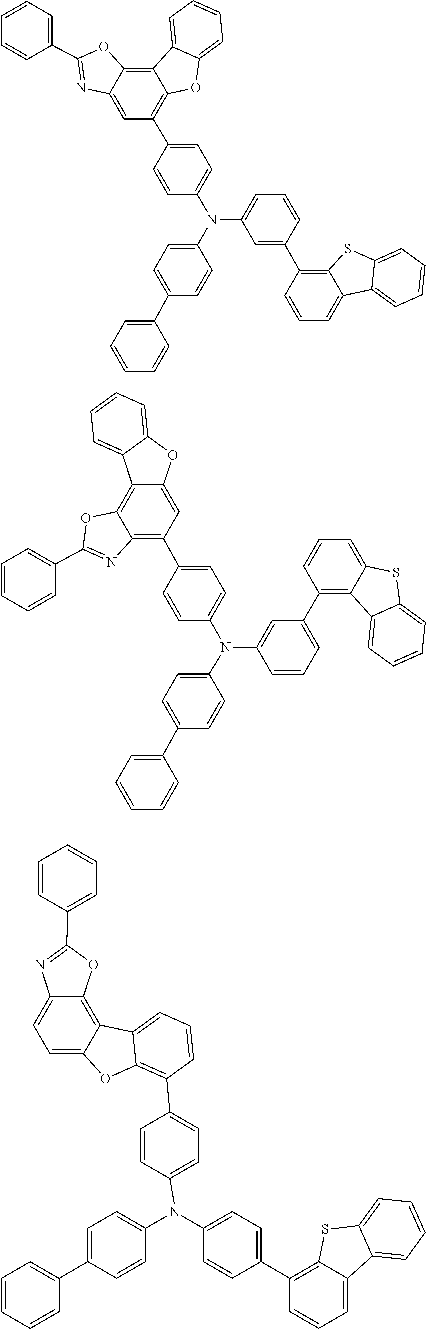

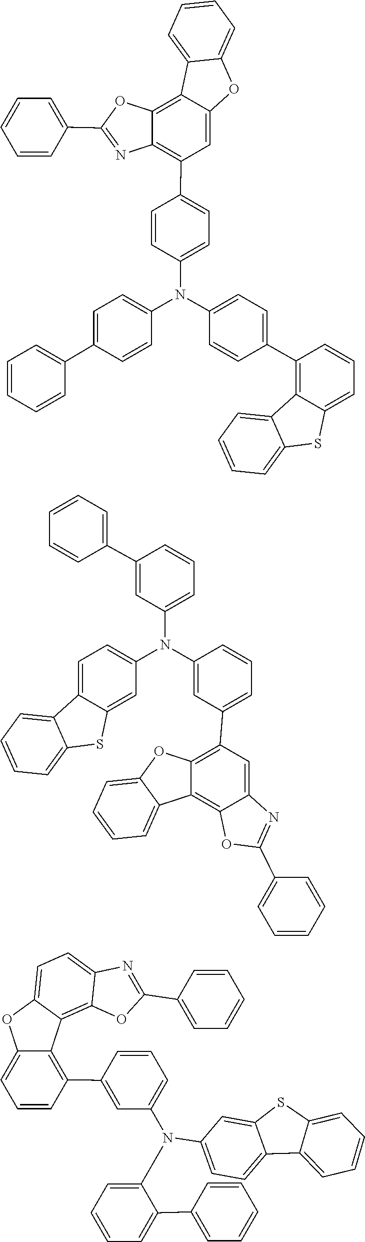 [SMILES: c1ccc(-c2ccc(N(c3ccc(-c4cc5oc6ccccc6c5c5oc(-c6ccccc6)nc45)cc3)c3ccc(-c4cccc5sc6ccccc6c45)cc3)cc2)cc1 ; c1ccc(-c2cccc(N(c3cccc(-c4cc5nc(-c6ccccc6)oc5c5c4oc4ccccc45)c3)c3ccc4c(c3)sc3ccccc34)c2)cc1 ; c1ccc(-c2nc3ccc4oc5cccc(-c6cccc(N(c7ccc8c(c7)sc7ccccc78)c7ccccc7-c7ccccc7)c6)c5c4c3o2)cc1]

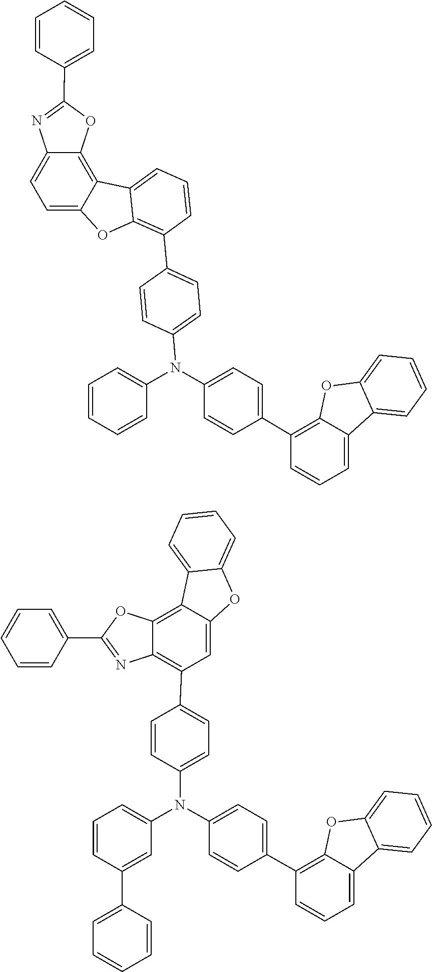 [SMILES: c1ccc(-c2nc3ccc4oc5c(-c6ccc(N(c7ccccc7)c7ccc(-c8cccc9c8oc8ccccc89)cc7)cc6)cccc5c4c3o2)cc1 ; c1ccc(-c2cccc(N(c3ccc(-c4cc5oc6ccccc6c5c5oc(-c6ccccc6)nc45)cc3)c3ccc(-c4cccc5c4oc4ccccc45)cc3)c2)cc1]

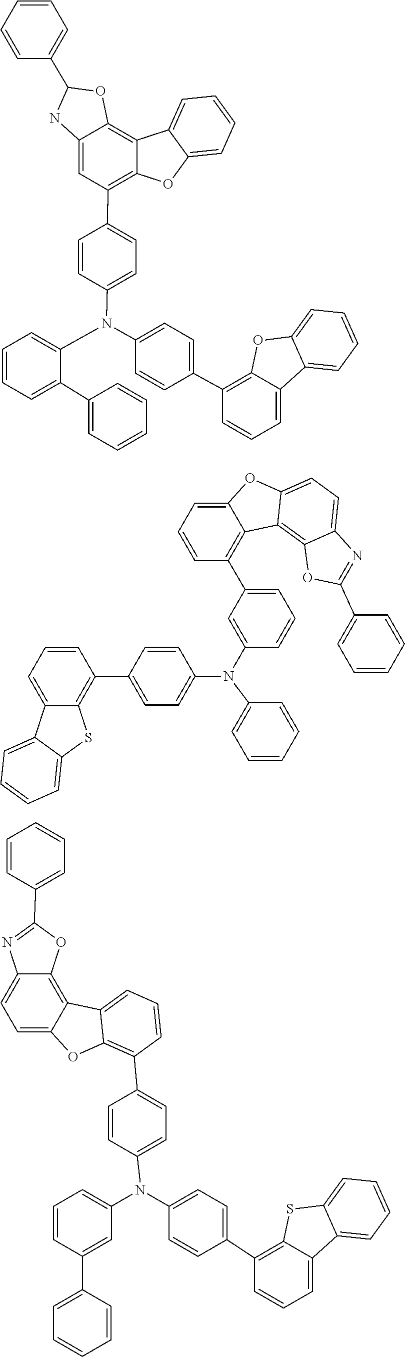

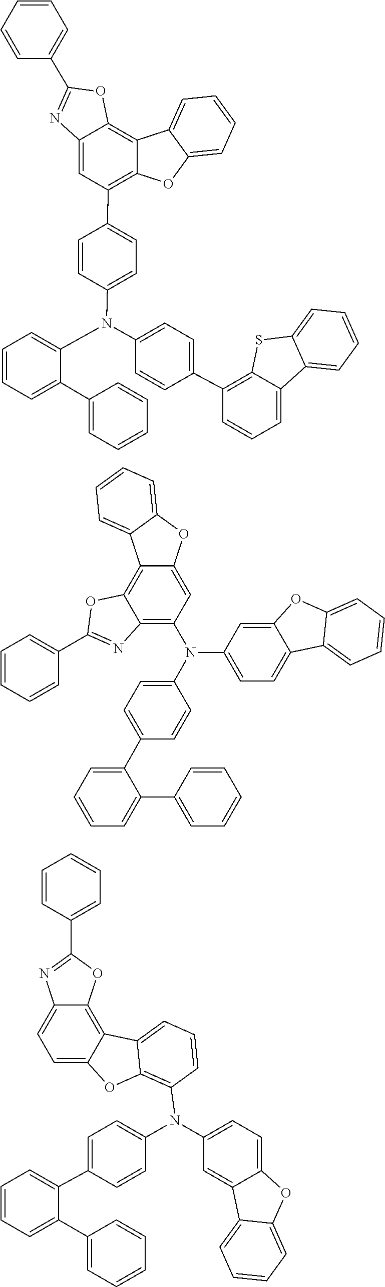

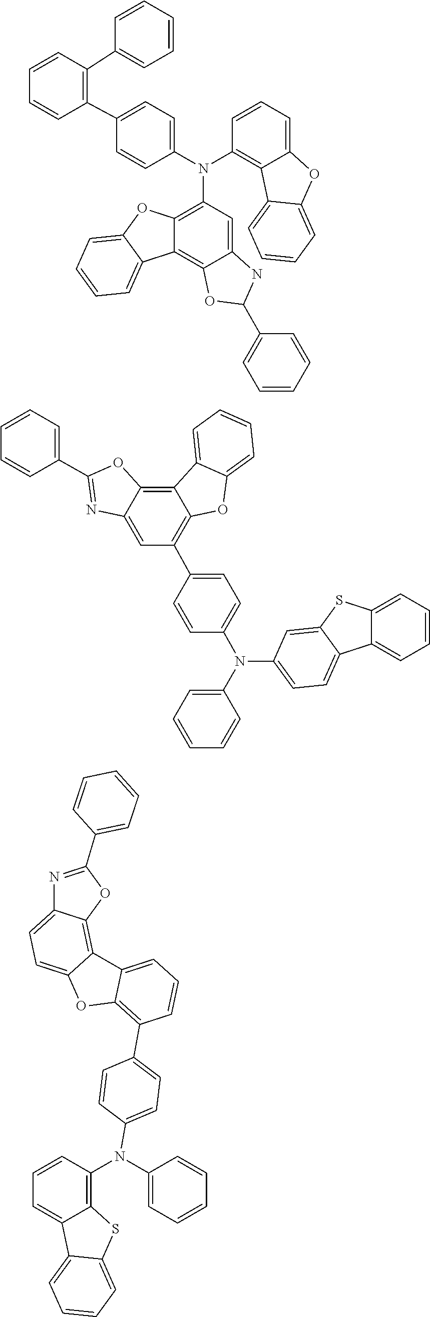

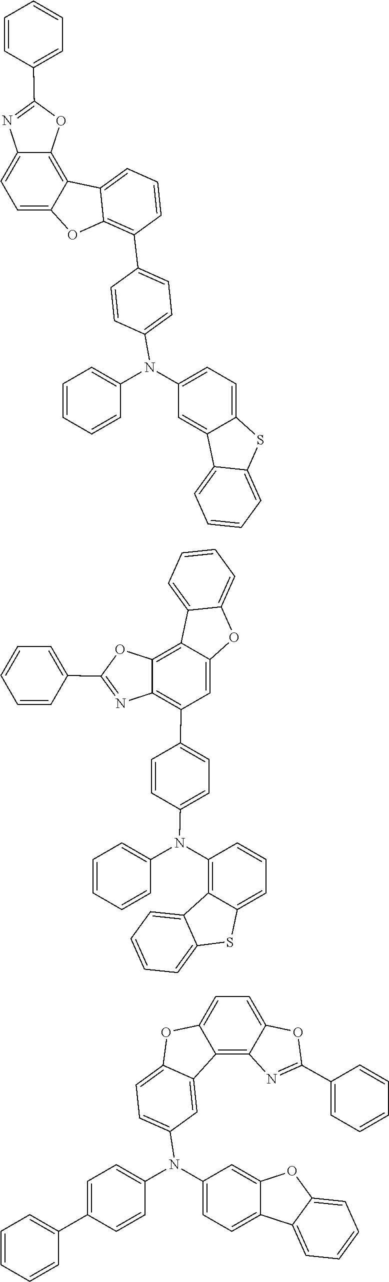

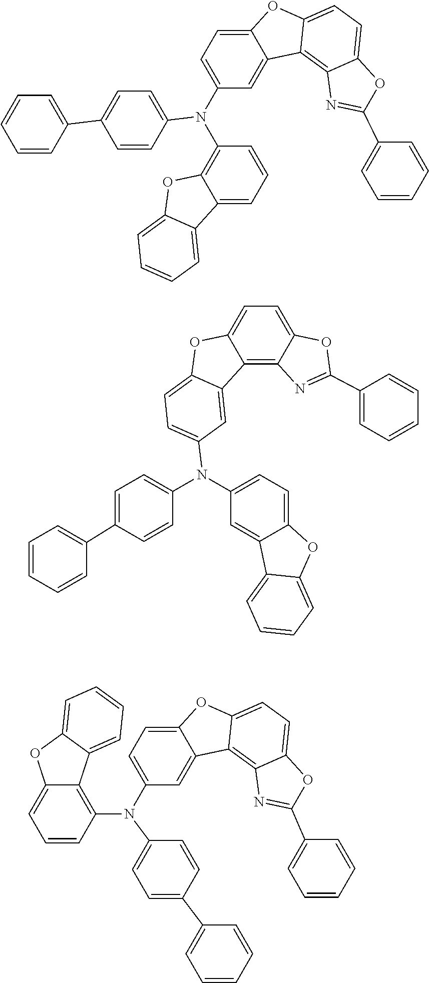 [SMILES: c1ccc(-c2ccc(N(c3ccc4oc5ccc6oc(-c7ccccc7)nc6c5c4c3)c3cccc4c3oc3ccccc34)cc2)cc1 ; c1ccc(-c2ccc(N(c3ccc4oc5ccccc5c4c3)c3ccc4oc5ccc6oc(-c7ccccc7)nc6c5c4c3)cc2)cc1 ; c1ccc(-c2ccc(N(c3ccc4oc5ccc6oc(-c7ccccc7)nc6c5c4c3)c3cccc4oc5ccccc5c34)cc2)cc1]

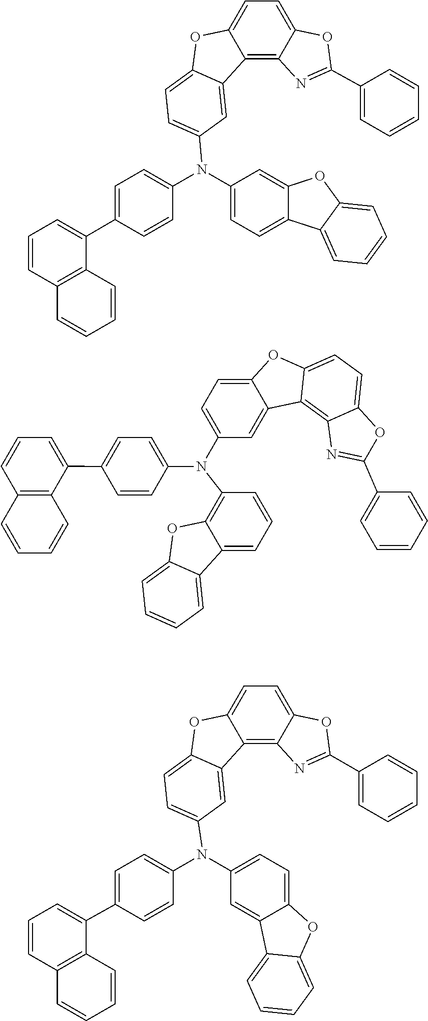 [SMILES: c1ccc(-c2nc3c(ccc4oc5ccc(N(c6ccc(-c7cccc8ccccc78)cc6)c6ccc7c(c6)oc6ccccc67)cc5c43)o2)cc1 ; c1ccc(-c2nc3c(ccc4oc5ccc(N(c6ccc(-c7cccc8ccccc78)cc6)c6cccc7c6oc6ccccc67)cc5c43)o2)cc1 ; c1ccc(-c2nc3c(ccc4oc5ccc(N(c6ccc(-c7cccc8ccccc78)cc6)c6ccc7oc8ccccc8c7c6)cc5c43)o2)cc1]

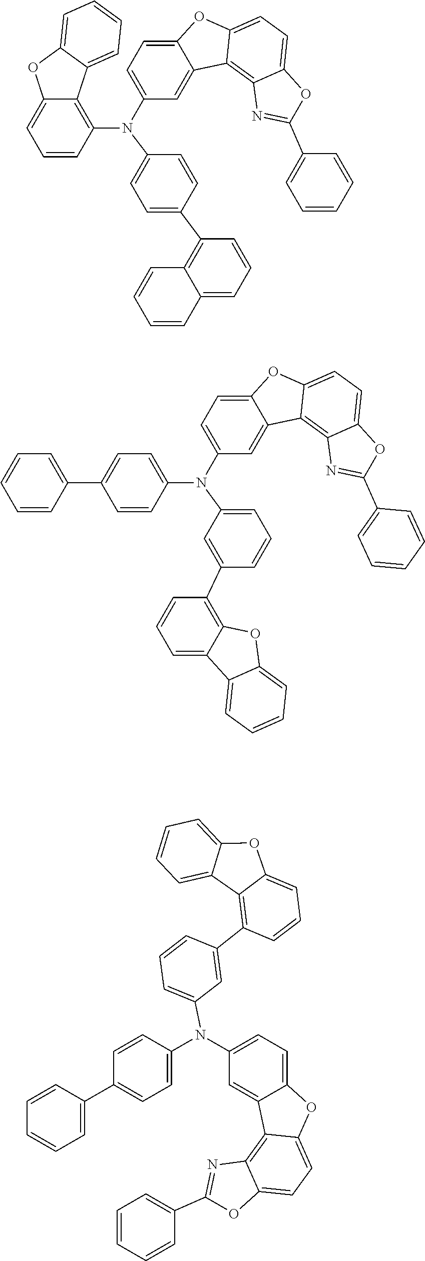 [SMILES: c1ccc(-c2nc3c(ccc4oc5ccc(N(c6ccc(-c7cccc8ccccc78)cc6)c6cccc7oc8ccccc8c67)cc5c43)o2)cc1 ; c1ccc(-c2ccc(N(c3cccc(-c4cccc5c4oc4ccccc45)c3)c3ccc4oc5ccc6oc(-c7ccccc7)nc6c5c4c3)cc2)cc1 ; c1ccc(-c2ccc(N(c3cccc(-c4cccc5oc6ccccc6c45)c3)c3ccc4oc5ccc6oc(-c7ccccc7)nc6c5c4c3)cc2)cc1]

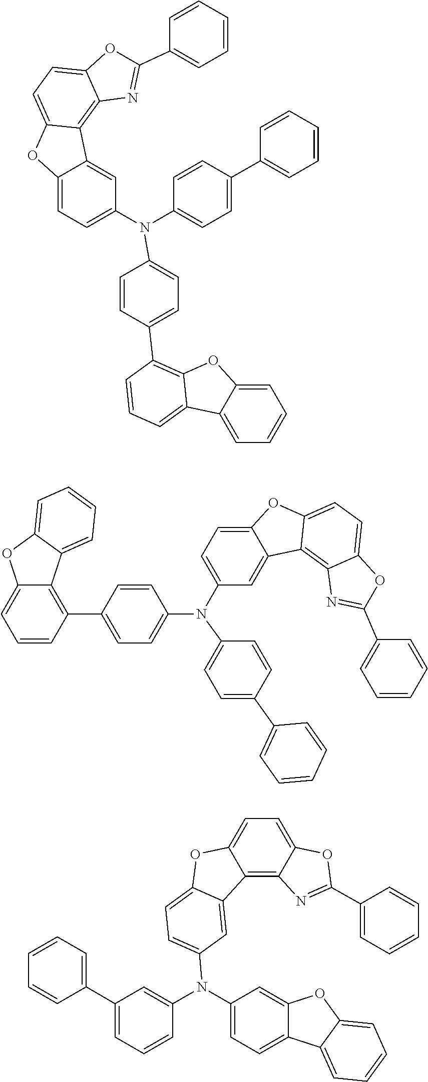

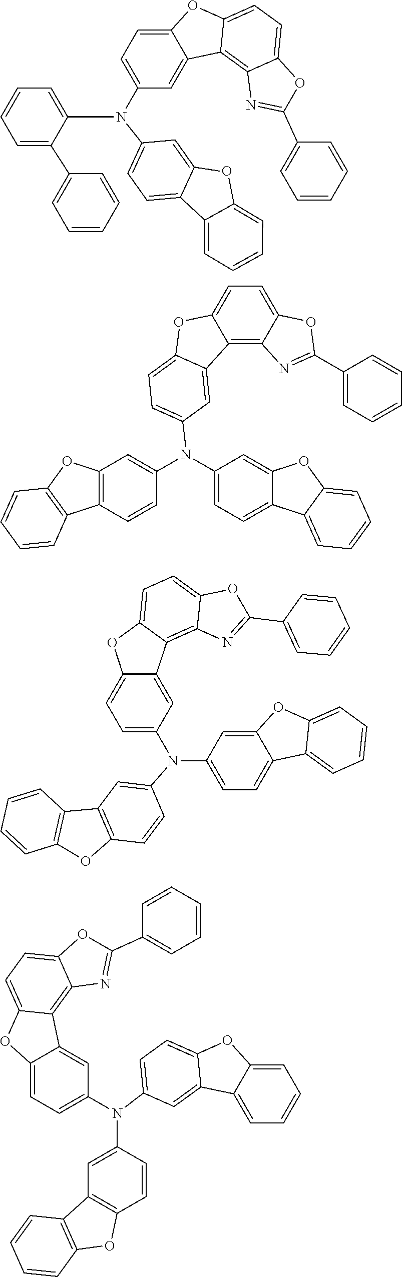

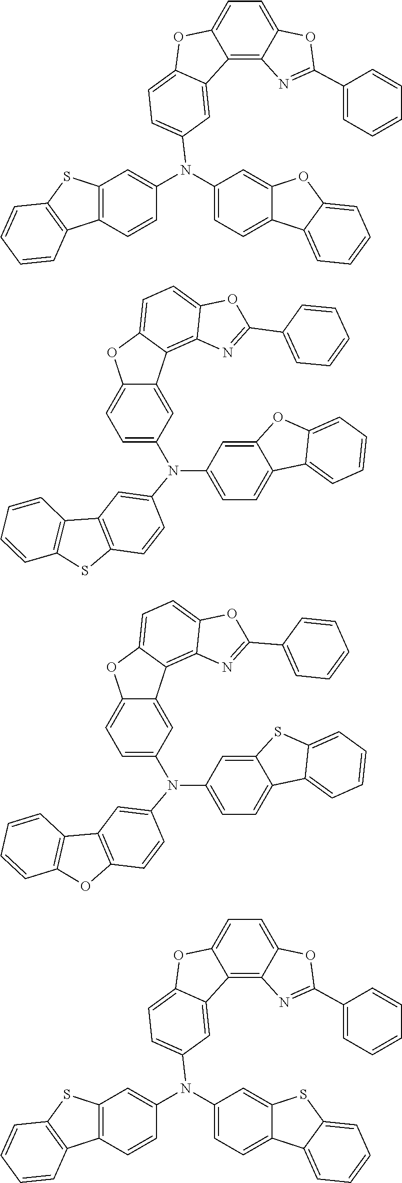 [SMILES: c1ccc(-c2nc3c(ccc4oc5ccc(N(c6ccc7c(c6)oc6ccccc67)c6ccc7c(c6)sc6ccccc67)cc5c43)o2)cc1 ; c1ccc(-c2nc3c(ccc4oc5ccc(N(c6ccc7c(c6)oc6ccccc67)c6ccc7sc8ccccc8c7c6)cc5c43)o2)cc1 ; c1ccc(-c2nc3c(ccc4oc5ccc(N(c6ccc7c(c6)sc6ccccc67)c6ccc7oc8ccccc8c7c6)cc5c43)o2)cc1 ; c1ccc(-c2nc3c(ccc4oc5ccc(N(c6ccc7c(c6)sc6ccccc67)c6ccc7c(c6)sc6ccccc67)cc5c43)o2)cc1]

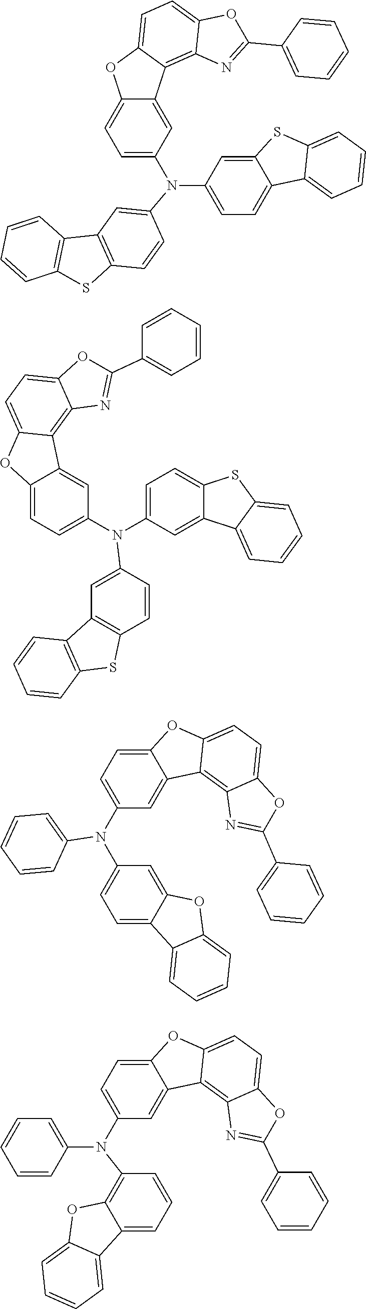

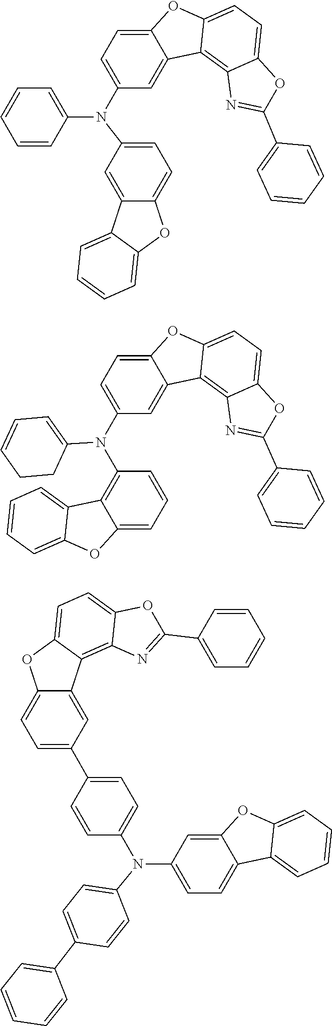

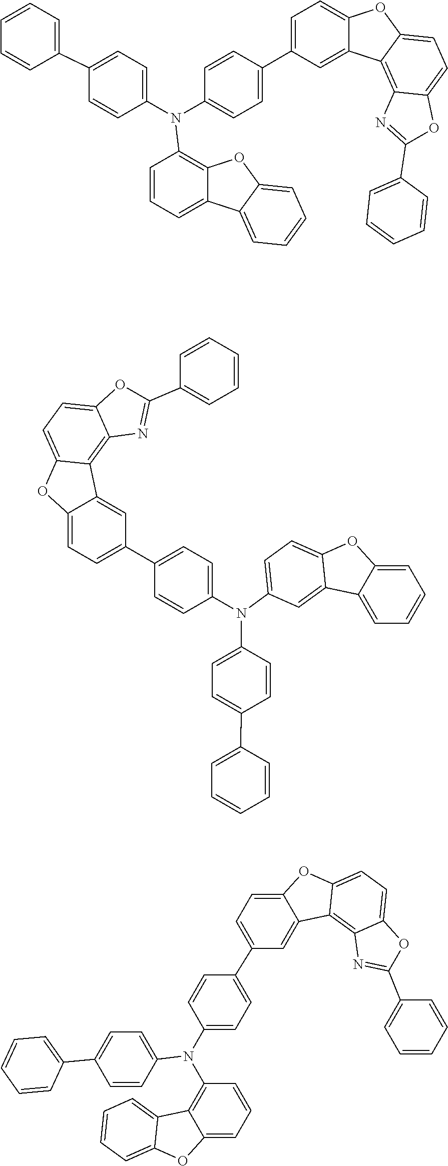 [SMILES: c1ccc(-c2ccc(N(c3ccc(-c4ccc5oc6ccc7oc(-c8ccccc8)nc7c6c5c4)cc3)c3cccc4c3oc3ccccc34)cc2)cc1 ; c1ccc(-c2ccc(N(c3ccc(-c4ccc5oc6ccc7oc(-c8ccccc8)nc7c6c5c4)cc3)c3ccc4oc5ccccc5c4c3)cc2)cc1 ; c1ccc(-c2ccc(N(c3ccc(-c4ccc5oc6ccc7oc(-c8ccccc8)nc7c6c5c4)cc3)c3cccc4oc5ccccc5c34)cc2)cc1]

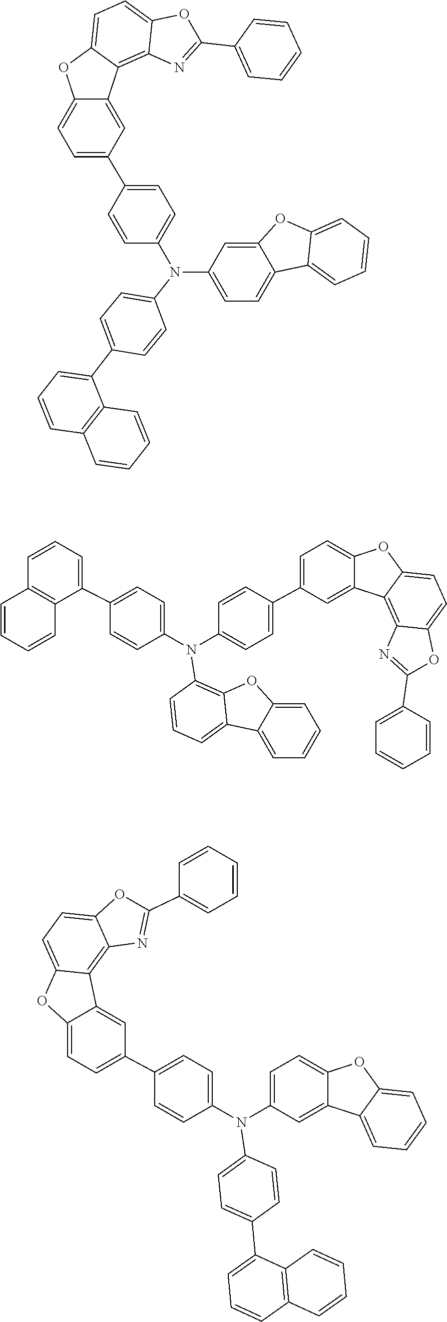 [SMILES: c1ccc(-c2nc3c(ccc4oc5ccc(-c6ccc(N(c7ccc(-c8cccc9ccccc89)cc7)c7ccc8c(c7)oc7ccccc78)cc6)cc5c43)o2)cc1 ; c1ccc(-c2nc3c(ccc4oc5ccc(-c6ccc(N(c7ccc(-c8cccc9ccccc89)cc7)c7cccc8c7oc7ccccc78)cc6)cc5c43)o2)cc1 ; c1ccc(-c2nc3c(ccc4oc5ccc(-c6ccc(N(c7ccc(-c8cccc9ccccc89)cc7)c7ccc8oc9ccccc9c8c7)cc6)cc5c43)o2)cc1]

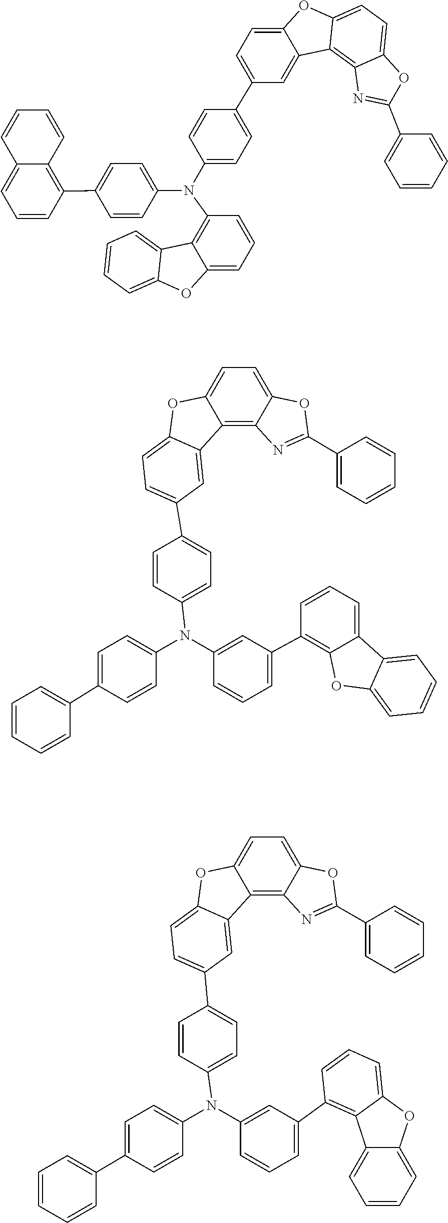 [SMILES: c1ccc(-c2nc3c(ccc4oc5ccc(-c6ccc(N(c7ccc(-c8cccc9ccccc89)cc7)c7cccc8oc9ccccc9c78)cc6)cc5c43)o2)cc1 ; c1ccc(-c2ccc(N(c3ccc(-c4ccc5oc6ccc7oc(-c8ccccc8)nc7c6c5c4)cc3)c3cccc(-c4cccc5c4oc4ccccc45)c3)cc2)cc1 ; c1ccc(-c2ccc(N(c3ccc(-c4ccc5oc6ccc7oc(-c8ccccc8)nc7c6c5c4)cc3)c3cccc(-c4cccc5oc6ccccc6c45)c3)cc2)cc1]

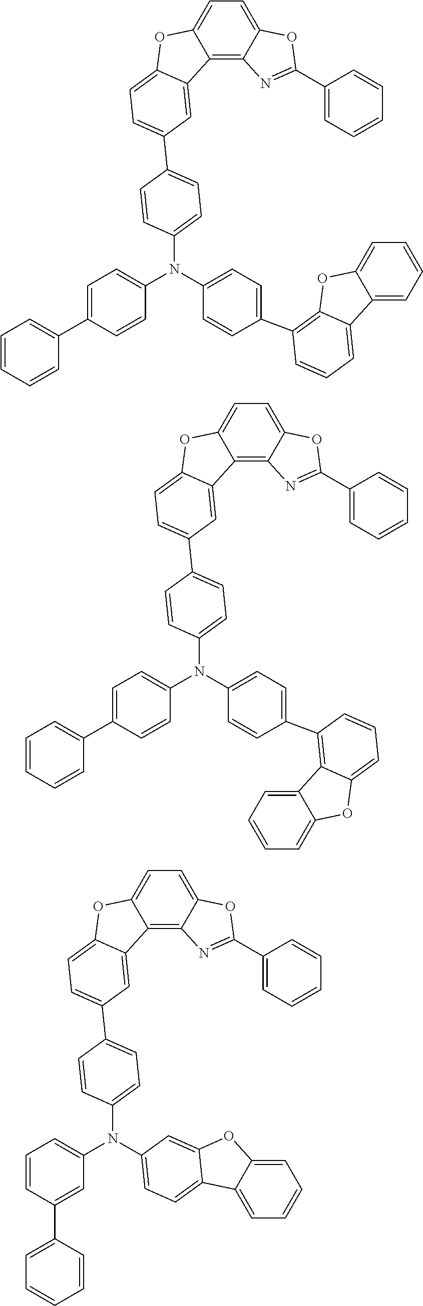 [SMILES: c1ccc(-c2ccc(N(c3ccc(-c4ccc5oc6ccc7oc(-c8ccccc8)nc7c6c5c4)cc3)c3ccc(-c4cccc5c4oc4ccccc45)cc3)cc2)cc1 ; c1ccc(-c2ccc(N(c3ccc(-c4ccc5oc6ccc7oc(-c8ccccc8)nc7c6c5c4)cc3)c3ccc(-c4cccc5oc6ccccc6c45)cc3)cc2)cc1 ; c1ccc(-c2cccc(N(c3ccc(-c4ccc5oc6ccc7oc(-c8ccccc8)nc7c6c5c4)cc3)c3ccc4c(c3)oc3ccccc34)c2)cc1]

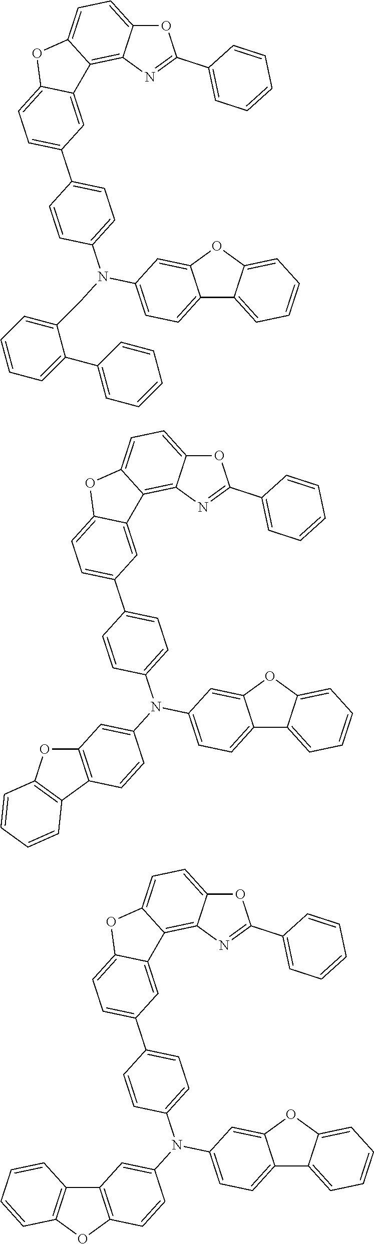

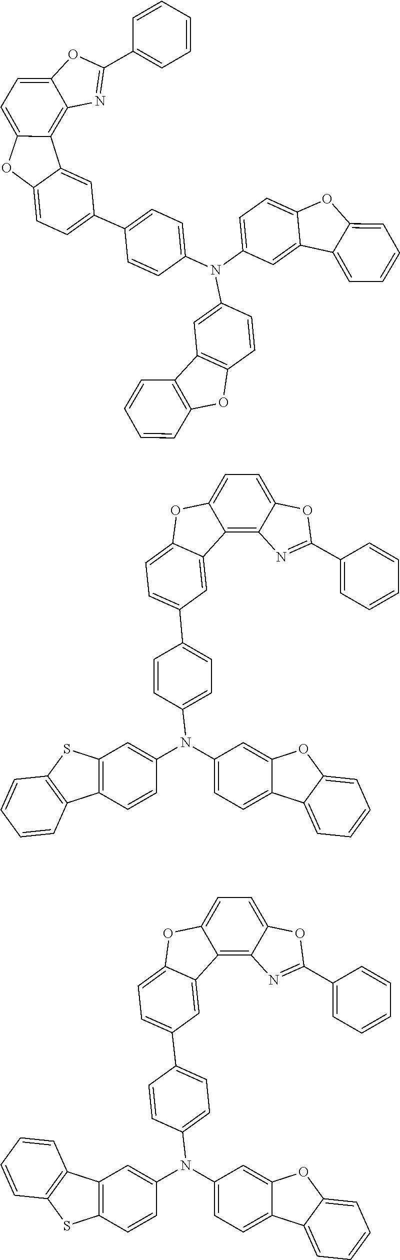 [SMILES: c1ccc(-c2nc3c(ccc4oc5ccc(-c6ccc(N(c7ccc8oc9ccccc9c8c7)c7ccc8oc9ccccc9c8c7)cc6)cc5c43)o2)cc1 ; c1ccc(-c2nc3c(ccc4oc5ccc(-c6ccc(N(c7ccc8c(c7)oc7ccccc78)c7ccc8c(c7)sc7ccccc78)cc6)cc5c43)o2)cc1 ; c1ccc(-c2nc3c(ccc4oc5ccc(-c6ccc(N(c7ccc8c(c7)oc7ccccc78)c7ccc8sc9ccccc9c8c7)cc6)cc5c43)o2)cc1]

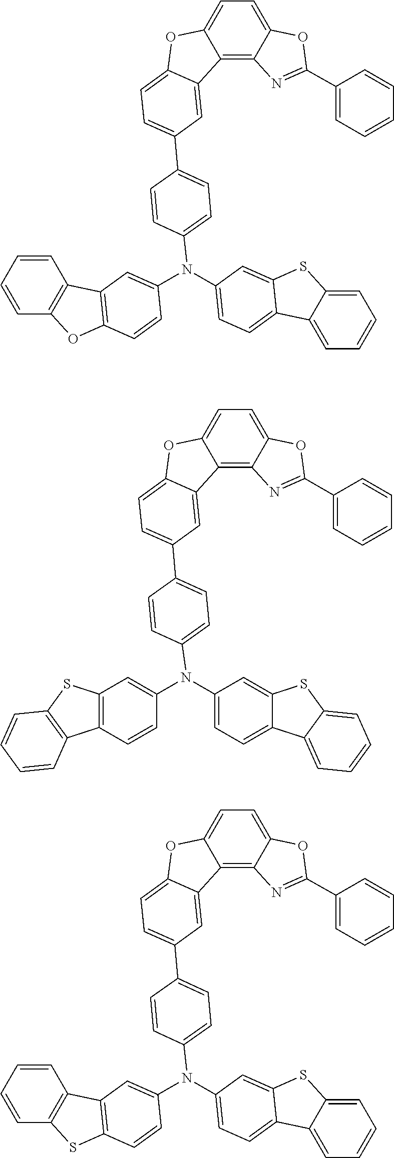 [SMILES: c1ccc(-c2nc3c(ccc4oc5ccc(-c6ccc(N(c7ccc8c(c7)sc7ccccc78)c7ccc8oc9ccccc9c8c7)cc6)cc5c43)o2)cc1 ; c1ccc(-c2nc3c(ccc4oc5ccc(-c6ccc(N(c7ccc8c(c7)sc7ccccc78)c7ccc8c(c7)sc7ccccc78)cc6)cc5c43)o2)cc1 ; c1ccc(-c2nc3c(ccc4oc5ccc(-c6ccc(N(c7ccc8c(c7)sc7ccccc78)c7ccc8sc9ccccc9c8c7)cc6)cc5c43)o2)cc1]

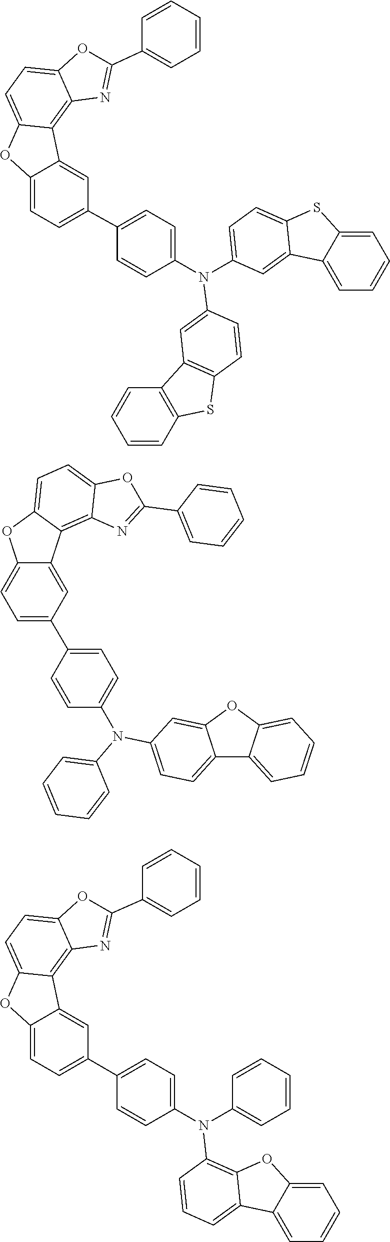

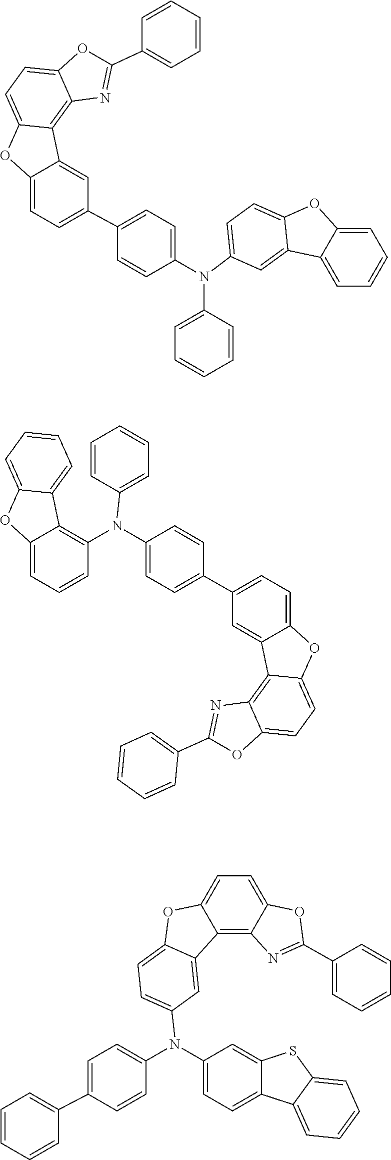 [SMILES: c1ccc(-c2nc3c(ccc4oc5ccc(-c6ccc(N(c7ccccc7)c7ccc8oc9ccccc9c8c7)cc6)cc5c43)o2)cc1 ; c1ccc(-c2nc3c(ccc4oc5ccc(-c6ccc(N(c7ccccc7)c7cccc8oc9ccccc9c78)cc6)cc5c43)o2)cc1 ; c1ccc(-c2ccc(N(c3ccc4c(c3)sc3ccccc34)c3ccc4oc5ccc6oc(-c7ccccc7)nc6c5c4c3)cc2)cc1]

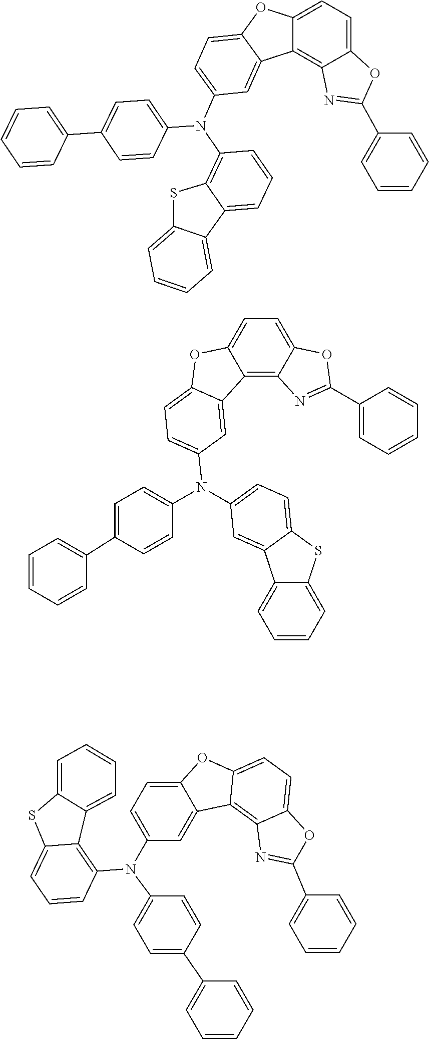 [SMILES: c1ccc(-c2ccc(N(c3ccc4oc5ccc6oc(-c7ccccc7)nc6c5c4c3)c3cccc4c3sc3ccccc34)cc2)cc1 ; c1ccc(-c2ccc(N(c3ccc4sc5ccccc5c4c3)c3ccc4oc5ccc6oc(-c7ccccc7)nc6c5c4c3)cc2)cc1 ; c1ccc(-c2ccc(N(c3ccc4oc5ccc6oc(-c7ccccc7)nc6c5c4c3)c3cccc4sc5ccccc5c34)cc2)cc1]

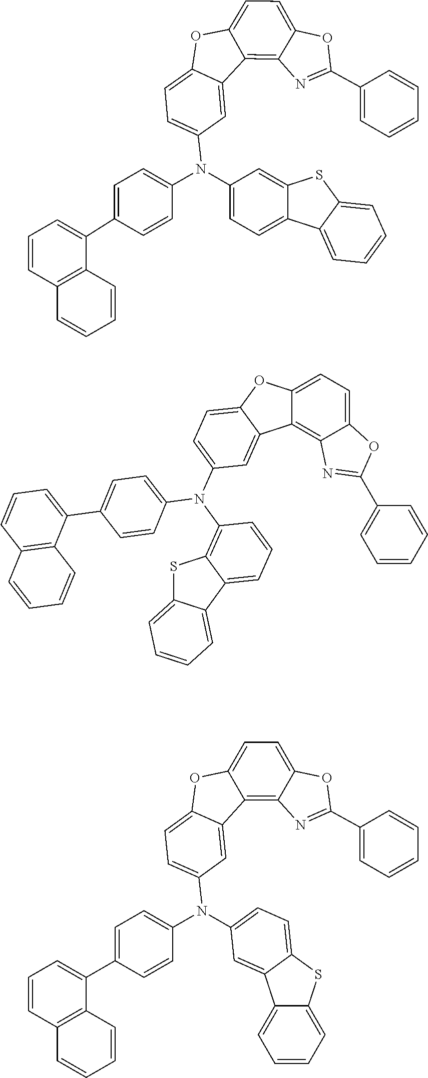 [SMILES: c1ccc(-c2nc3c(ccc4oc5ccc(N(c6ccc(-c7cccc8ccccc78)cc6)c6ccc7c(c6)sc6ccccc67)cc5c43)o2)cc1 ; c1ccc(-c2nc3c(ccc4oc5ccc(N(c6ccc(-c7cccc8ccccc78)cc6)c6cccc7c6sc6ccccc67)cc5c43)o2)cc1 ; c1ccc(-c2nc3c(ccc4oc5ccc(N(c6ccc(-c7cccc8ccccc78)cc6)c6ccc7sc8ccccc8c7c6)cc5c43)o2)cc1]

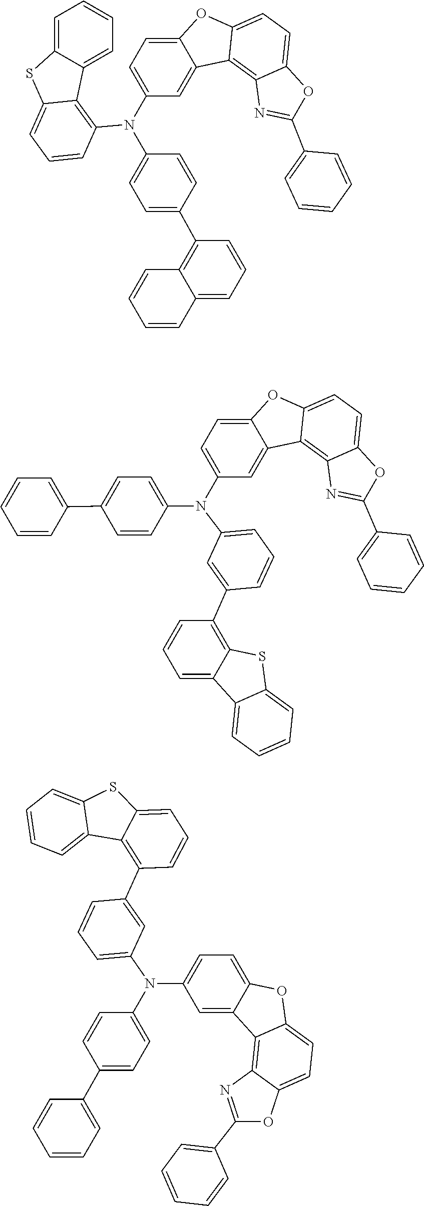 [SMILES: c1ccc(-c2nc3c(ccc4oc5ccc(N(c6ccc(-c7cccc8ccccc78)cc6)c6cccc7sc8ccccc8c67)cc5c43)o2)cc1 ; c1ccc(-c2ccc(N(c3cccc(-c4cccc5c4sc4ccccc45)c3)c3ccc4oc5ccc6oc(-c7ccccc7)nc6c5c4c3)cc2)cc1 ; c1ccc(-c2ccc(N(c3cccc(-c4cccc5sc6ccccc6c45)c3)c3ccc4oc5ccc6oc(-c7ccccc7)nc6c5c4c3)cc2)cc1]

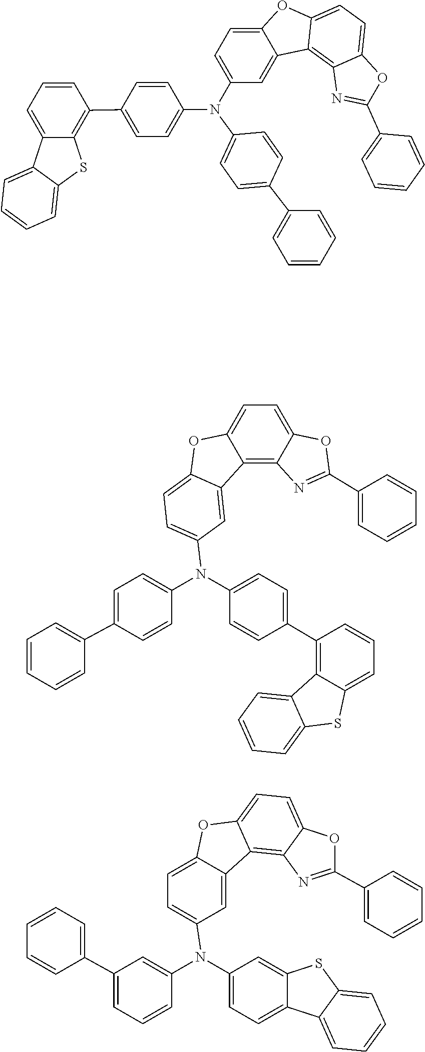

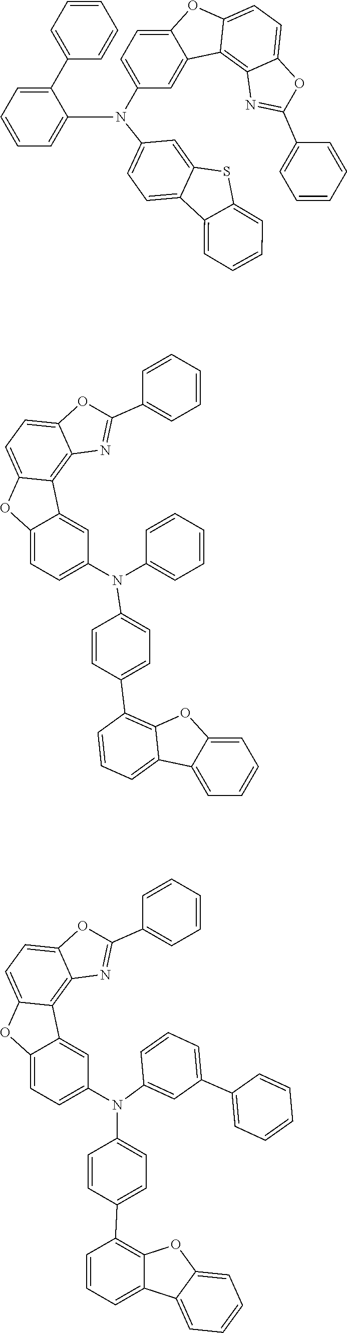 [SMILES: c1ccc(-c2nc3c(ccc4oc5ccc(N(c6ccc7c(c6)sc6ccccc67)c6ccccc6-c6ccccc6)cc5c43)o2)cc1 ; c1ccc(-c2nc3c(ccc4oc5ccc(N(c6ccccc6)c6ccc(-c7cccc8c7oc7ccccc78)cc6)cc5c43)o2)cc1 ; c1ccc(-c2cccc(N(c3ccc(-c4cccc5c4oc4ccccc45)cc3)c3ccc4oc5ccc6oc(-c7ccccc7)nc6c5c4c3)c2)cc1]

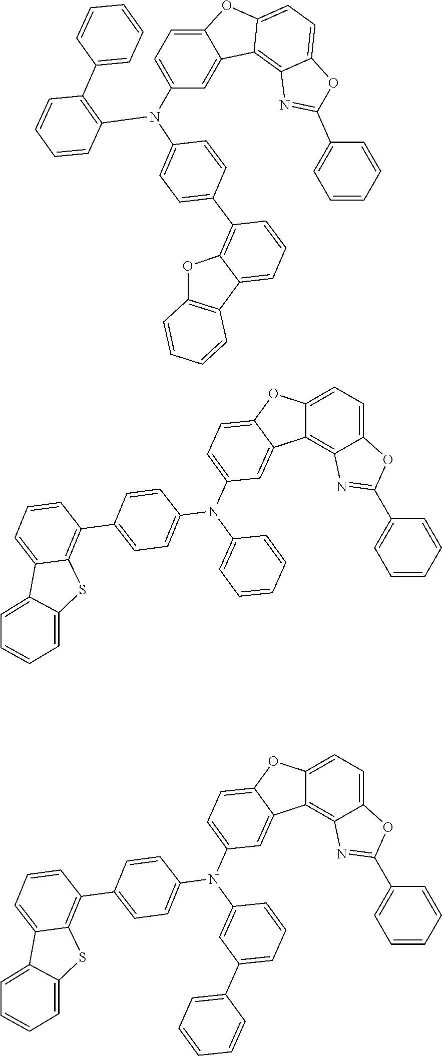 [SMILES: c1ccc(-c2nc3c(ccc4oc5ccc(N(c6ccc(-c7cccc8c7oc7ccccc78)cc6)c6ccccc6-c6ccccc6)cc5c43)o2)cc1 ; c1ccc(-c2nc3c(ccc4oc5ccc(N(c6ccccc6)c6ccc(-c7cccc8c7sc7ccccc78)cc6)cc5c43)o2)cc1 ; c1ccc(-c2cccc(N(c3ccc(-c4cccc5c4sc4ccccc45)cc3)c3ccc4oc5ccc6oc(-c7ccccc7)nc6c5c4c3)c2)cc1]

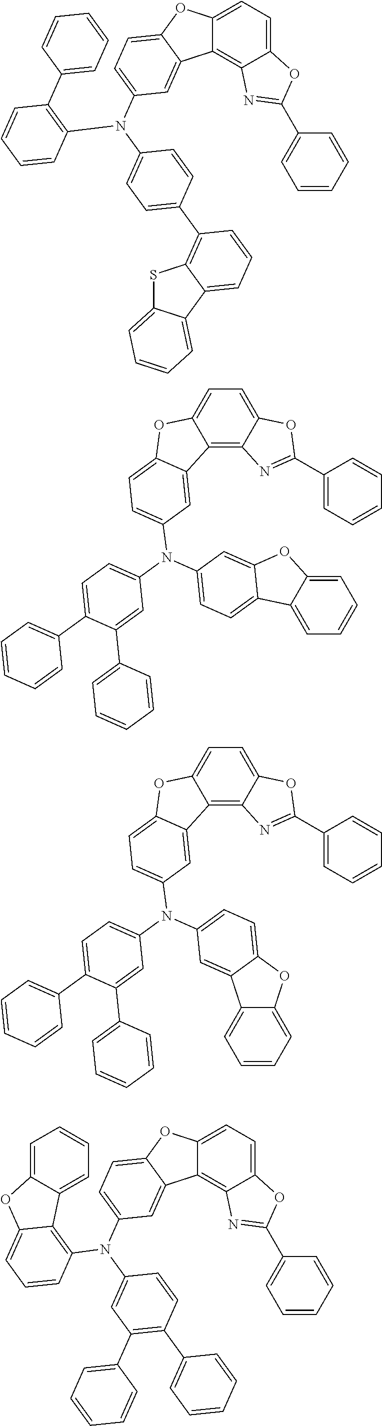

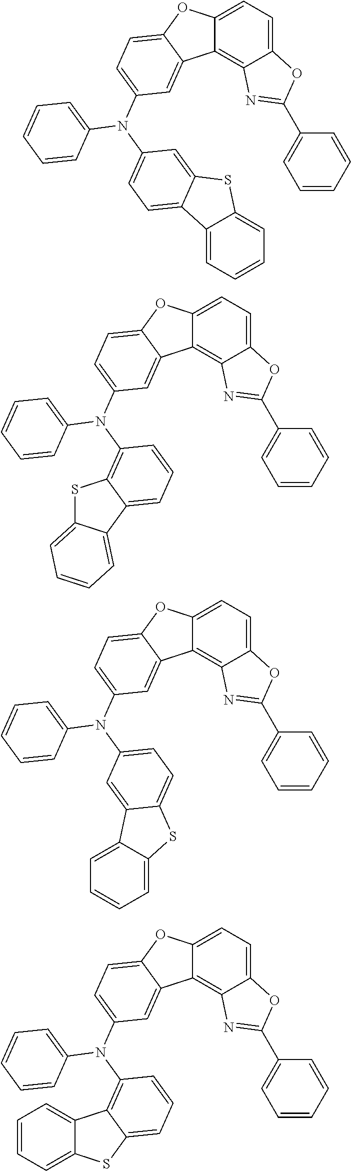

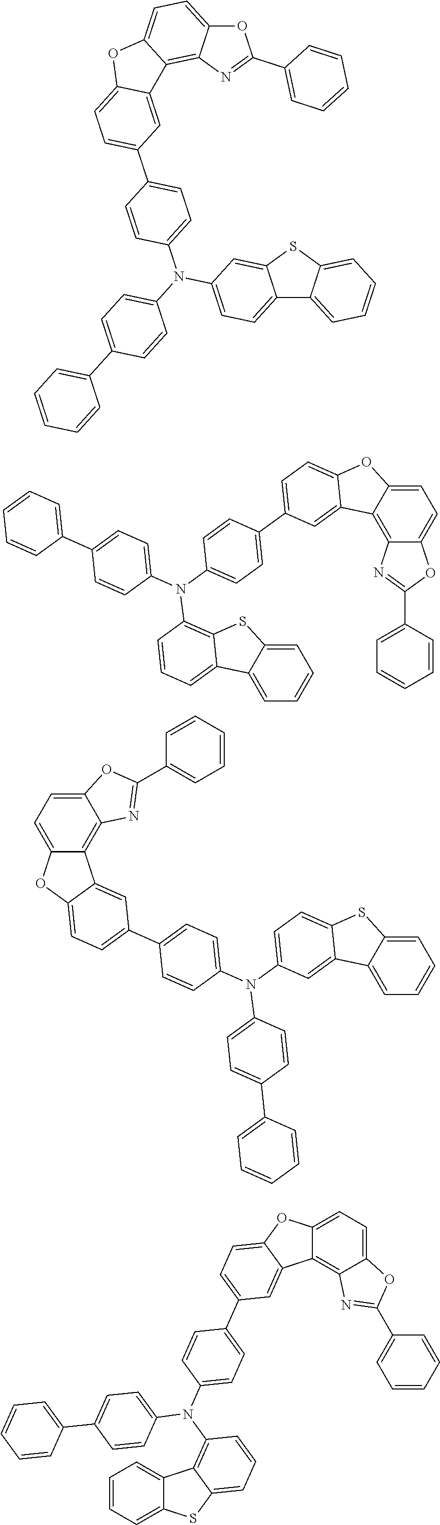

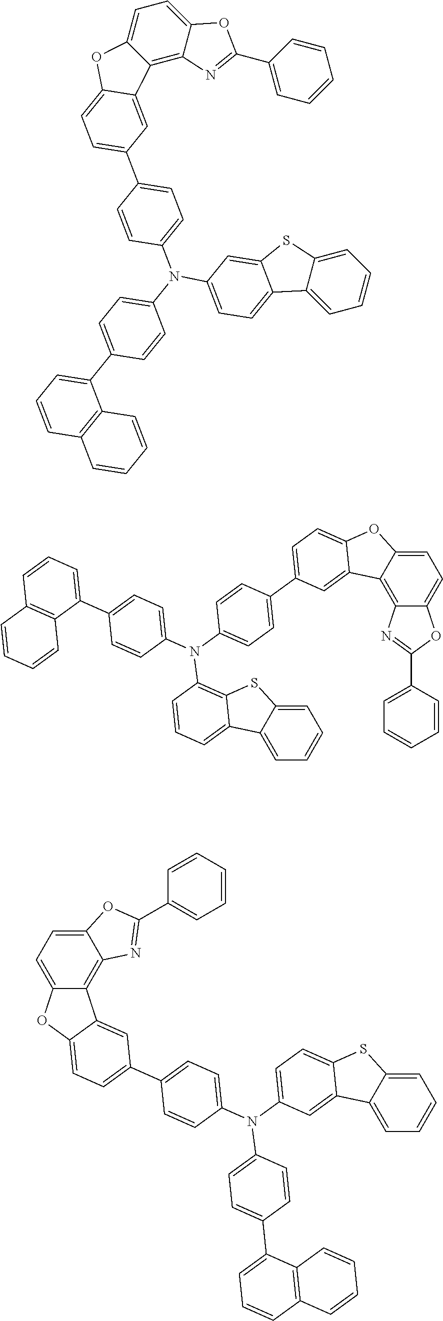 [SMILES: c1ccc(-c2nc3c(ccc4oc5ccc(-c6ccc(N(c7ccc(-c8cccc9ccccc89)cc7)c7ccc8c(c7)sc7ccccc78)cc6)cc5c43)o2)cc1 ; c1ccc(-c2nc3c(ccc4oc5ccc(-c6ccc(N(c7ccc(-c8cccc9ccccc89)cc7)c7cccc8c7sc7ccccc78)cc6)cc5c43)o2)cc1 ; c1ccc(-c2nc3c(ccc4oc5ccc(-c6ccc(N(c7ccc(-c8cccc9ccccc89)cc7)c7ccc8sc9ccccc9c8c7)cc6)cc5c43)o2)cc1]

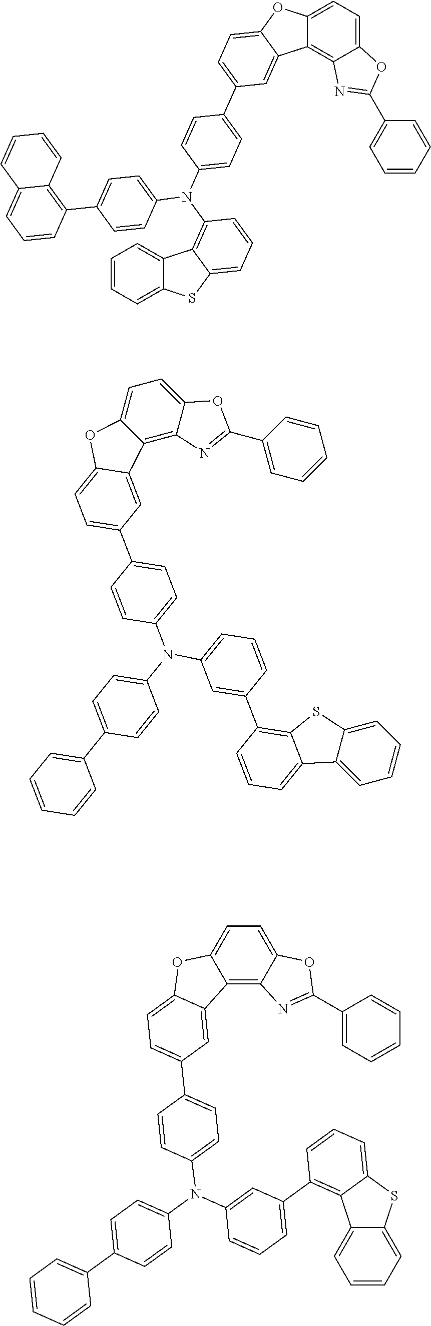 [SMILES: c1ccc(-c2nc3c(ccc4oc5ccc(-c6ccc(N(c7ccc(-c8cccc9ccccc89)cc7)c7cccc8sc9ccccc9c78)cc6)cc5c43)o2)cc1 ; c1ccc(-c2ccc(N(c3ccc(-c4ccc5oc6ccc7oc(-c8ccccc8)nc7c6c5c4)cc3)c3cccc(-c4cccc5c4sc4ccccc45)c3)cc2)cc1 ; c1ccc(-c2ccc(N(c3ccc(-c4ccc5oc6ccc7oc(-c8ccccc8)nc7c6c5c4)cc3)c3cccc(-c4cccc5sc6ccccc6c45)c3)cc2)cc1]

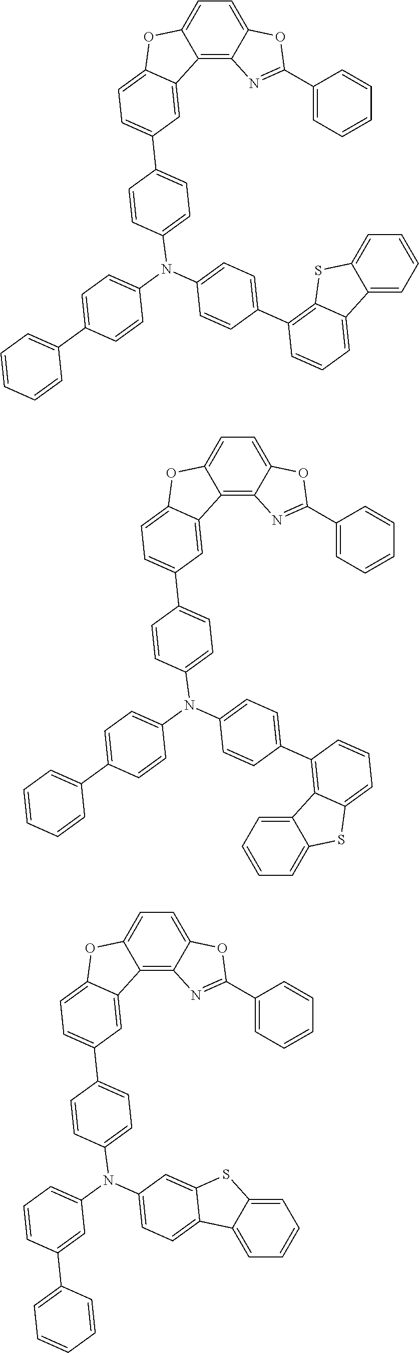 [SMILES: c1ccc(-c2ccc(N(c3ccc(-c4ccc5oc6ccc7oc(-c8ccccc8)nc7c6c5c4)cc3)c3ccc(-c4cccc5c4sc4ccccc45)cc3)cc2)cc1 ; c1ccc(-c2ccc(N(c3ccc(-c4ccc5oc6ccc7oc(-c8ccccc8)nc7c6c5c4)cc3)c3ccc(-c4cccc5sc6ccccc6c45)cc3)cc2)cc1 ; c1ccc(-c2cccc(N(c3ccc(-c4ccc5oc6ccc7oc(-c8ccccc8)nc7c6c5c4)cc3)c3ccc4c(c3)sc3ccccc34)c2)cc1]

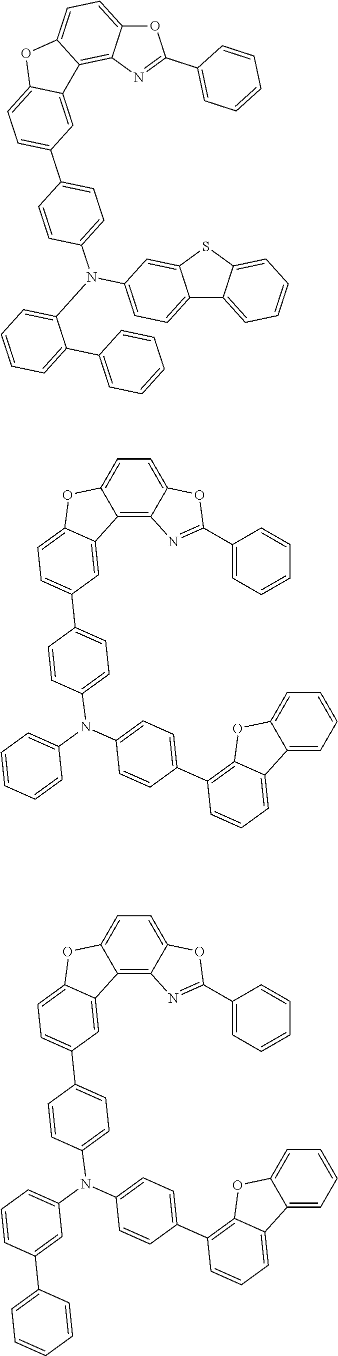 [SMILES: c1ccc(-c2nc3c(ccc4oc5ccc(-c6ccc(N(c7ccc8c(c7)sc7ccccc78)c7ccccc7-c7ccccc7)cc6)cc5c43)o2)cc1 ; c1ccc(-c2nc3c(ccc4oc5ccc(-c6ccc(N(c7ccccc7)c7ccc(-c8cccc9c8oc8ccccc89)cc7)cc6)cc5c43)o2)cc1 ; c1ccc(-c2cccc(N(c3ccc(-c4ccc5oc6ccc7oc(-c8ccccc8)nc7c6c5c4)cc3)c3ccc(-c4cccc5c4oc4ccccc45)cc3)c2)cc1]

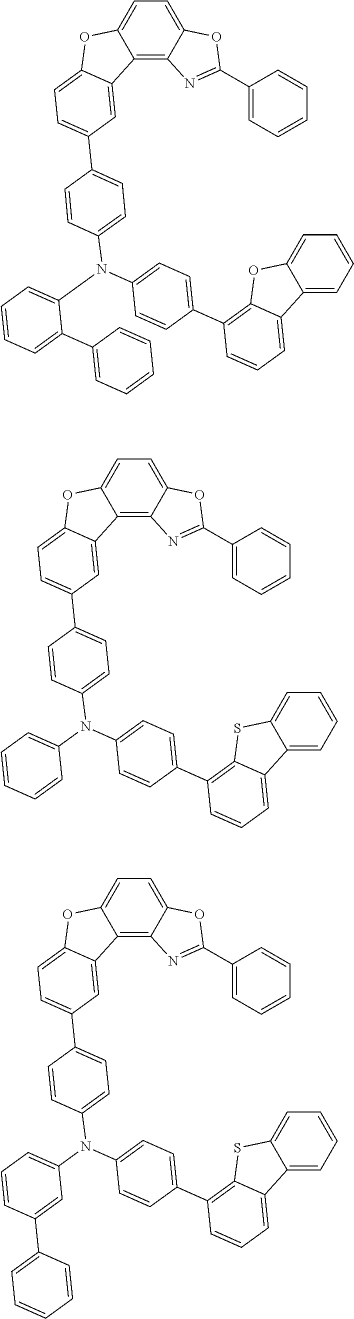 [SMILES: c1ccc(-c2nc3c(ccc4oc5ccc(-c6ccc(N(c7ccc(-c8cccc9c8oc8ccccc89)cc7)c7ccccc7-c7ccccc7)cc6)cc5c43)o2)cc1 ; c1ccc(-c2nc3c(ccc4oc5ccc(-c6ccc(N(c7ccccc7)c7ccc(-c8cccc9c8sc8ccccc89)cc7)cc6)cc5c43)o2)cc1 ; c1ccc(-c2cccc(N(c3ccc(-c4ccc5oc6ccc7oc(-c8ccccc8)nc7c6c5c4)cc3)c3ccc(-c4cccc5c4sc4ccccc45)cc3)c2)cc1]

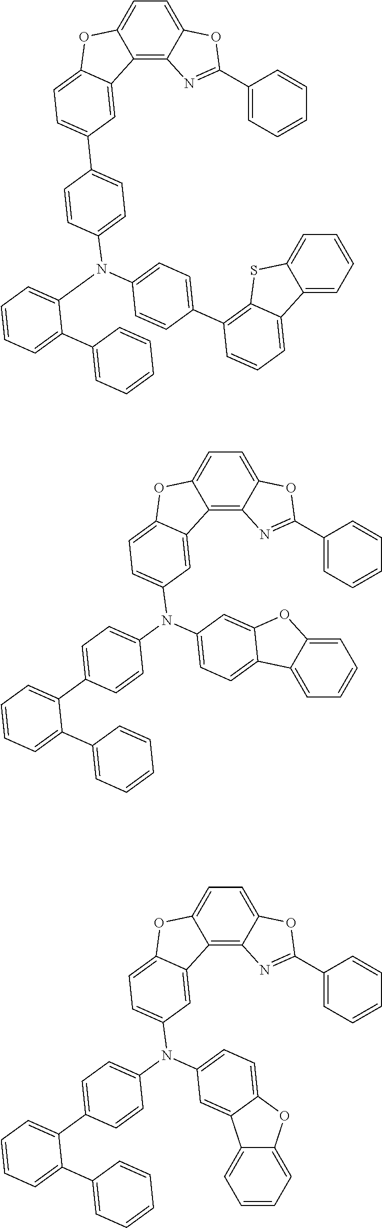 [SMILES: c1ccc(-c2nc3c(ccc4oc5ccc(-c6ccc(N(c7ccc(-c8cccc9c8sc8ccccc89)cc7)c7ccccc7-c7ccccc7)cc6)cc5c43)o2)cc1 ; c1ccc(-c2nc3c(ccc4oc5ccc(N(c6ccc(-c7ccccc7-c7ccccc7)cc6)c6ccc7c(c6)oc6ccccc67)cc5c43)o2)cc1 ; c1ccc(-c2nc3c(ccc4oc5ccc(N(c6ccc(-c7ccccc7-c7ccccc7)cc6)c6ccc7oc8ccccc8c7c6)cc5c43)o2)cc1]

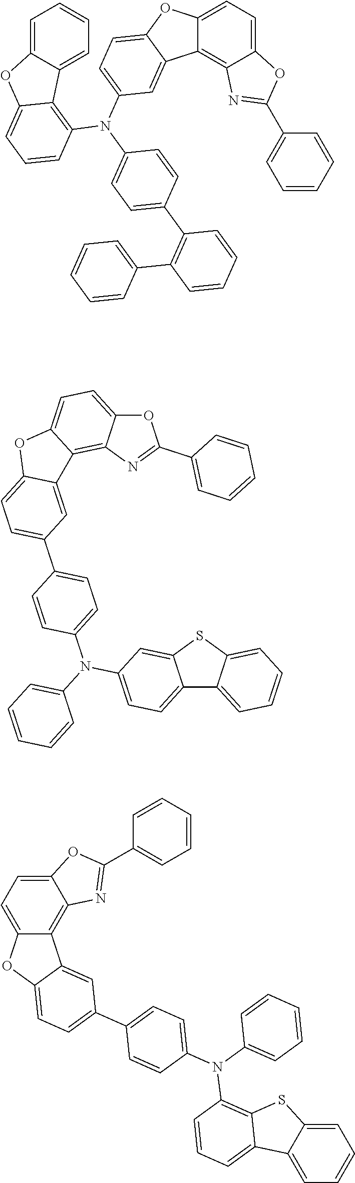 [SMILES: c1ccc(-c2nc3c(ccc4oc5ccc(N(c6ccc(-c7ccccc7-c7ccccc7)cc6)c6cccc7oc8ccccc8c67)cc5c43)o2)cc1 ; c1ccc(-c2nc3c(ccc4oc5ccc(-c6ccc(N(c7ccccc7)c7ccc8c(c7)sc7ccccc78)cc6)cc5c43)o2)cc1 ; c1ccc(-c2nc3c(ccc4oc5ccc(-c6ccc(N(c7ccccc7)c7cccc8c7sc7ccccc78)cc6)cc5c43)o2)cc1]

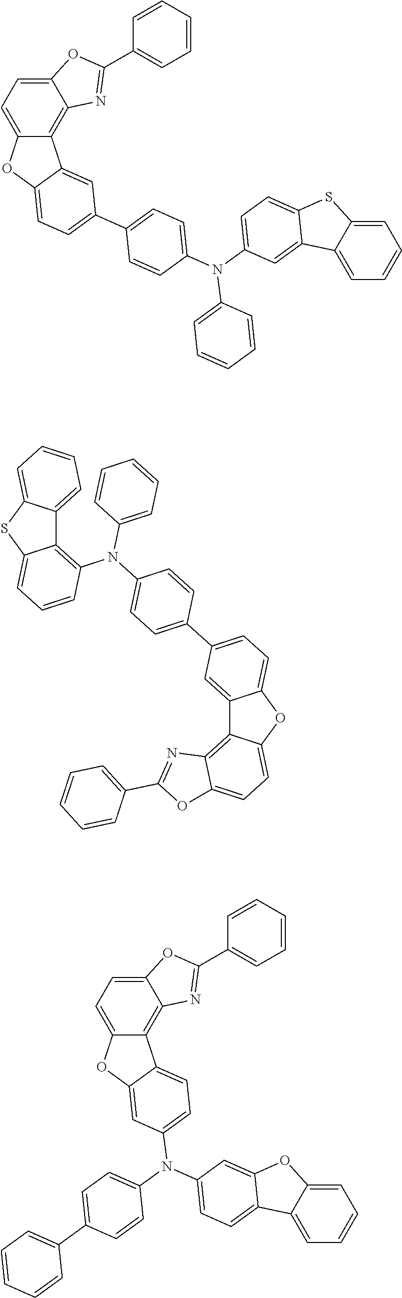 [SMILES: c1ccc(-c2nc3c(ccc4oc5ccc(-c6ccc(N(c7ccccc7)c7ccc8sc9ccccc9c8c7)cc6)cc5c43)o2)cc1 ; c1ccc(-c2nc3c(ccc4oc5ccc(-c6ccc(N(c7ccccc7)c7cccc8sc9ccccc9c78)cc6)cc5c43)o2)cc1 ; c1ccc(-c2ccc(N(c3ccc4c(c3)oc3ccccc34)c3ccc4c(c3)oc3ccc5oc(-c6ccccc6)nc5c34)cc2)cc1]

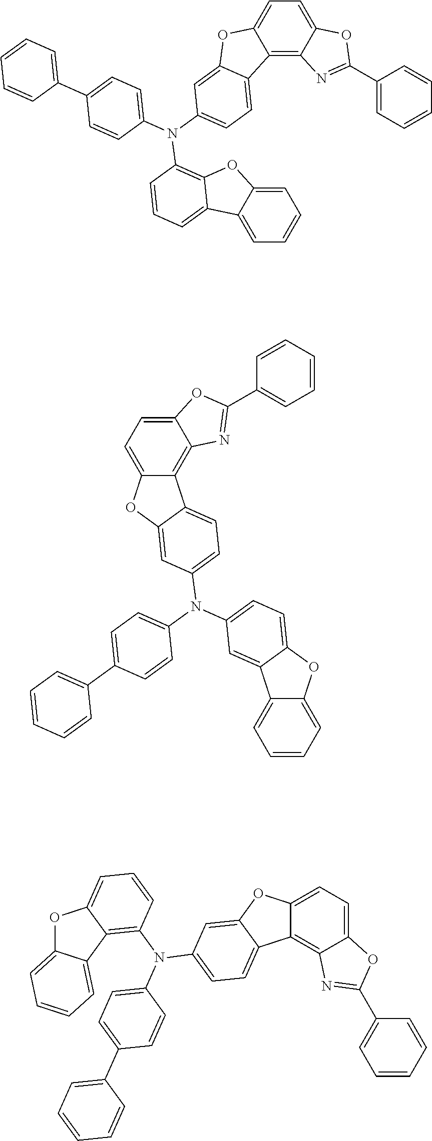 [SMILES: c1ccc(-c2ccc(N(c3ccc4c(c3)oc3ccc5oc(-c6ccccc6)nc5c34)c3cccc4c3oc3ccccc34)cc2)cc1 ; c1ccc(-c2ccc(N(c3ccc4c(c3)oc3ccc5oc(-c6ccccc6)nc5c34)c3ccc4oc5ccccc5c4c3)cc2)cc1 ; c1ccc(-c2ccc(N(c3ccc4c(c3)oc3ccc5oc(-c6ccccc6)nc5c34)c3cccc4oc5ccccc5c34)cc2)cc1]

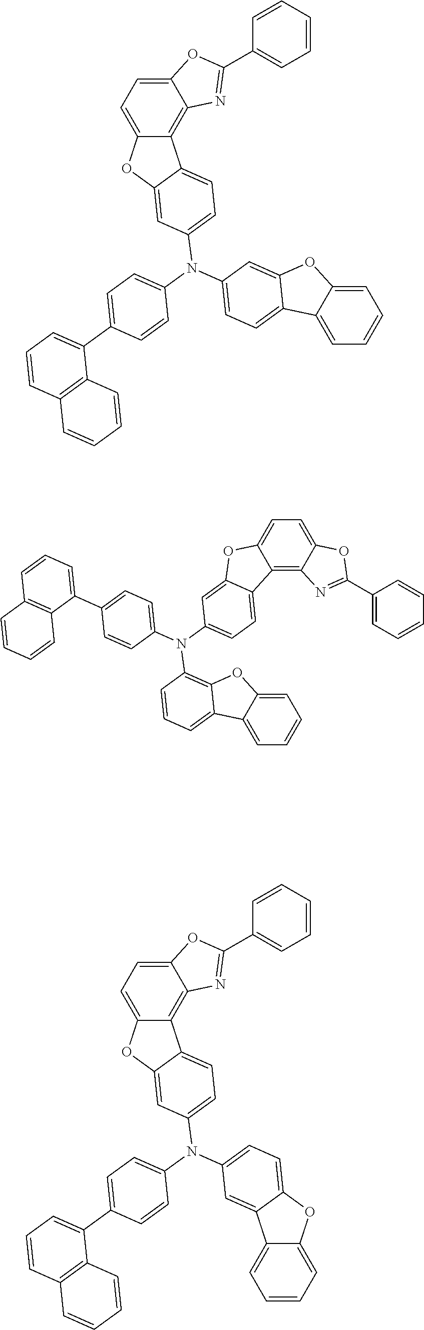 [SMILES: c1ccc(-c2nc3c(ccc4oc5cc(N(c6ccc(-c7cccc8ccccc78)cc6)c6ccc7c(c6)oc6ccccc67)ccc5c43)o2)cc1 ; c1ccc(-c2nc3c(ccc4oc5cc(N(c6ccc(-c7cccc8ccccc78)cc6)c6cccc7c6oc6ccccc67)ccc5c43)o2)cc1 ; c1ccc(-c2nc3c(ccc4oc5cc(N(c6ccc(-c7cccc8ccccc78)cc6)c6ccc7oc8ccccc8c7c6)ccc5c43)o2)cc1]

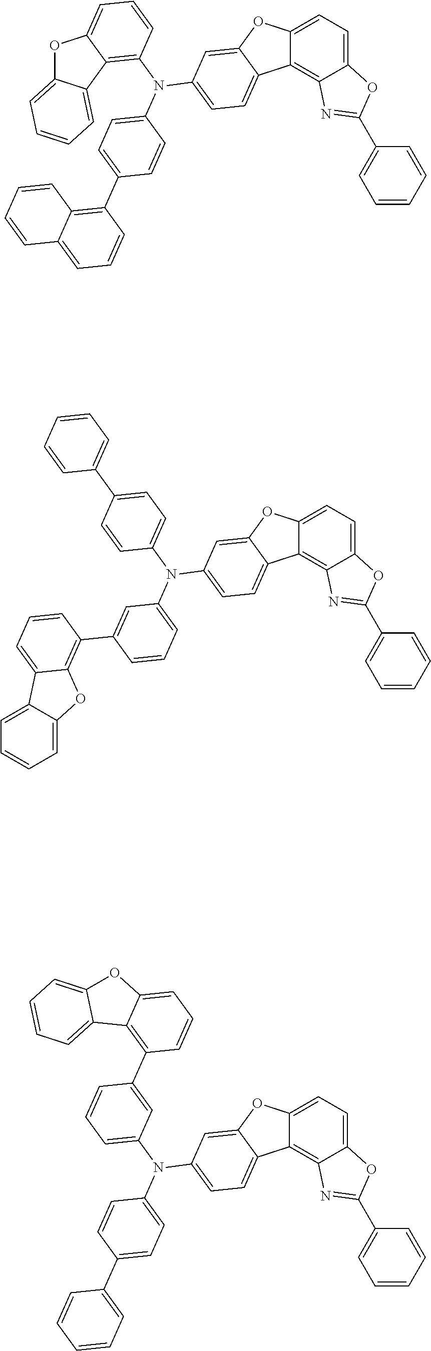 [SMILES: c1ccc(-c2nc3c(ccc4oc5cc(N(c6ccc(-c7cccc8ccccc78)cc6)c6cccc7oc8ccccc8c67)ccc5c43)o2)cc1 ; c1ccc(-c2ccc(N(c3cccc(-c4cccc5c4oc4ccccc45)c3)c3ccc4c(c3)oc3ccc5oc(-c6ccccc6)nc5c34)cc2)cc1 ; c1ccc(-c2ccc(N(c3cccc(-c4cccc5oc6ccccc6c45)c3)c3ccc4c(c3)oc3ccc5oc(-c6ccccc6)nc5c34)cc2)cc1]

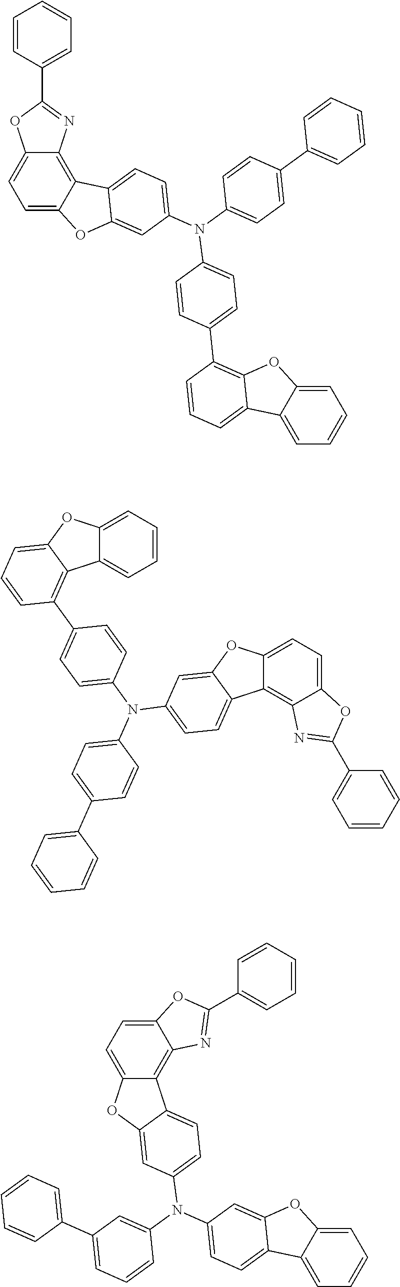 [SMILES: c1ccc(-c2ccc(N(c3ccc(-c4cccc5c4oc4ccccc45)cc3)c3ccc4c(c3)oc3ccc5oc(-c6ccccc6)nc5c34)cc2)cc1 ; c1ccc(-c2ccc(N(c3ccc(-c4cccc5oc6ccccc6c45)cc3)c3ccc4c(c3)oc3ccc5oc(-c6ccccc6)nc5c34)cc2)cc1 ; c1ccc(-c2cccc(N(c3ccc4c(c3)oc3ccccc34)c3ccc4c(c3)oc3ccc5oc(-c6ccccc6)nc5c34)c2)cc1]

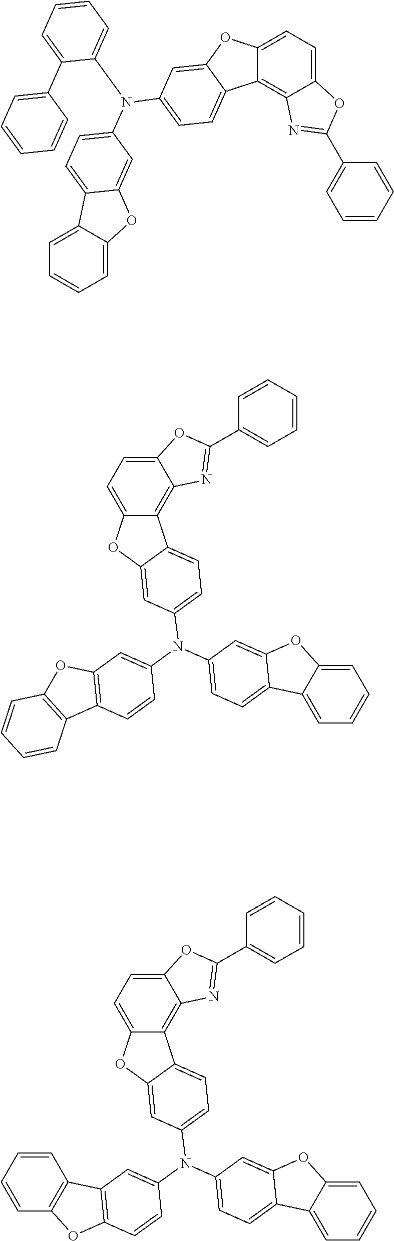 [SMILES: c1ccc(-c2nc3c(ccc4oc5cc(N(c6ccc7c(c6)oc6ccccc67)c6ccccc6-c6ccccc6)ccc5c43)o2)cc1 ; c1ccc(-c2nc3c(ccc4oc5cc(N(c6ccc7c(c6)oc6ccccc67)c6ccc7c(c6)oc6ccccc67)ccc5c43)o2)cc1 ; c1ccc(-c2nc3c(ccc4oc5cc(N(c6ccc7c(c6)oc6ccccc67)c6ccc7oc8ccccc8c7c6)ccc5c43)o2)cc1]

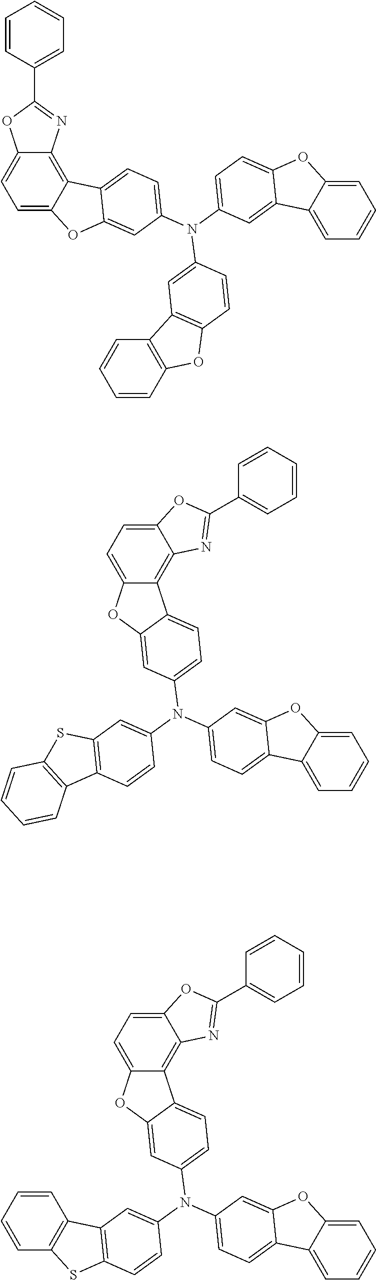 [SMILES: c1ccc(-c2nc3c(ccc4oc5cc(N(c6ccc7oc8ccccc8c7c6)c6ccc7oc8ccccc8c7c6)ccc5c43)o2)cc1 ; c1ccc(-c2nc3c(ccc4oc5cc(N(c6ccc7c(c6)oc6ccccc67)c6ccc7c(c6)sc6ccccc67)ccc5c43)o2)cc1 ; c1ccc(-c2nc3c(ccc4oc5cc(N(c6ccc7c(c6)oc6ccccc67)c6ccc7sc8ccccc8c7c6)ccc5c43)o2)cc1]

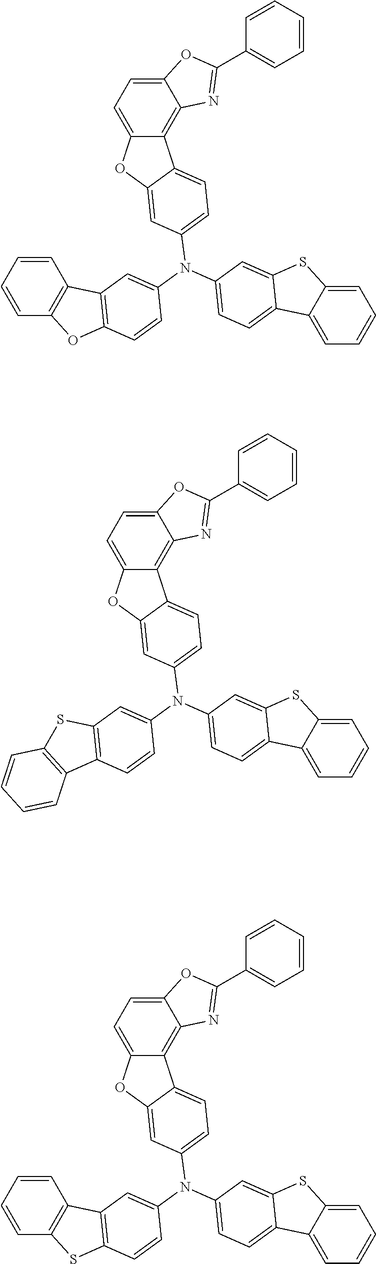 [SMILES: c1ccc(-c2nc3c(ccc4oc5cc(N(c6ccc7c(c6)sc6ccccc67)c6ccc7oc8ccccc8c7c6)ccc5c43)o2)cc1 ; c1ccc(-c2nc3c(ccc4oc5cc(N(c6ccc7c(c6)sc6ccccc67)c6ccc7c(c6)sc6ccccc67)ccc5c43)o2)cc1 ; c1ccc(-c2nc3c(ccc4oc5cc(N(c6ccc7c(c6)sc6ccccc67)c6ccc7sc8ccccc8c7c6)ccc5c43)o2)cc1]

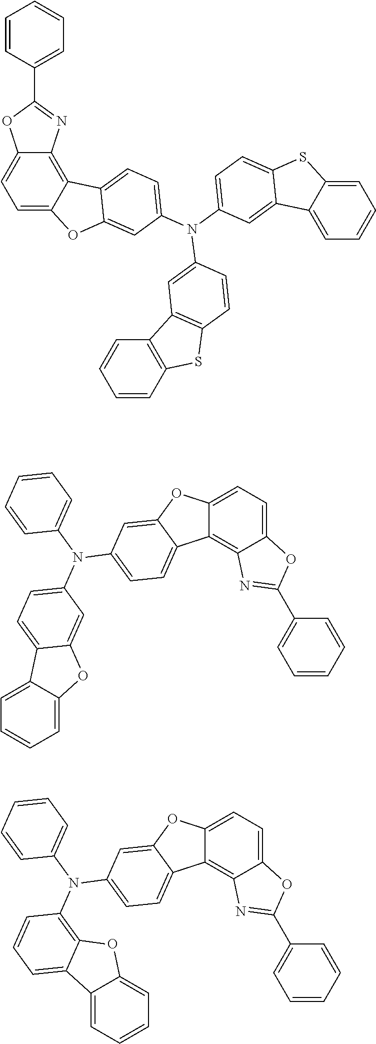 [SMILES: c1ccc(-c2nc3c(ccc4oc5cc(N(c6ccc7sc8ccccc8c7c6)c6ccc7sc8ccccc8c7c6)ccc5c43)o2)cc1 ; c1ccc(-c2nc3c(ccc4oc5cc(N(c6ccccc6)c6ccc7c(c6)oc6ccccc67)ccc5c43)o2)cc1 ; c1ccc(-c2nc3c(ccc4oc5cc(N(c6ccccc6)c6cccc7c6oc6ccccc67)ccc5c43)o2)cc1]

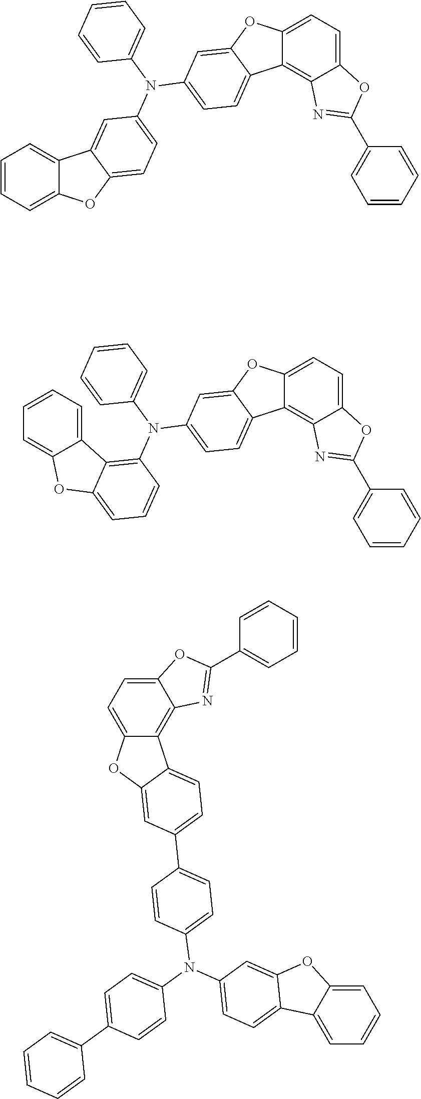 [SMILES: c1ccc(-c2nc3c(ccc4oc5cc(N(c6ccccc6)c6ccc7oc8ccccc8c7c6)ccc5c43)o2)cc1 ; c1ccc(-c2nc3c(ccc4oc5cc(N(c6ccccc6)c6cccc7oc8ccccc8c67)ccc5c43)o2)cc1 ; c1ccc(-c2ccc(N(c3ccc(-c4ccc5c(c4)oc4ccc6oc(-c7ccccc7)nc6c45)cc3)c3ccc4c(c3)oc3ccccc34)cc2)cc1]

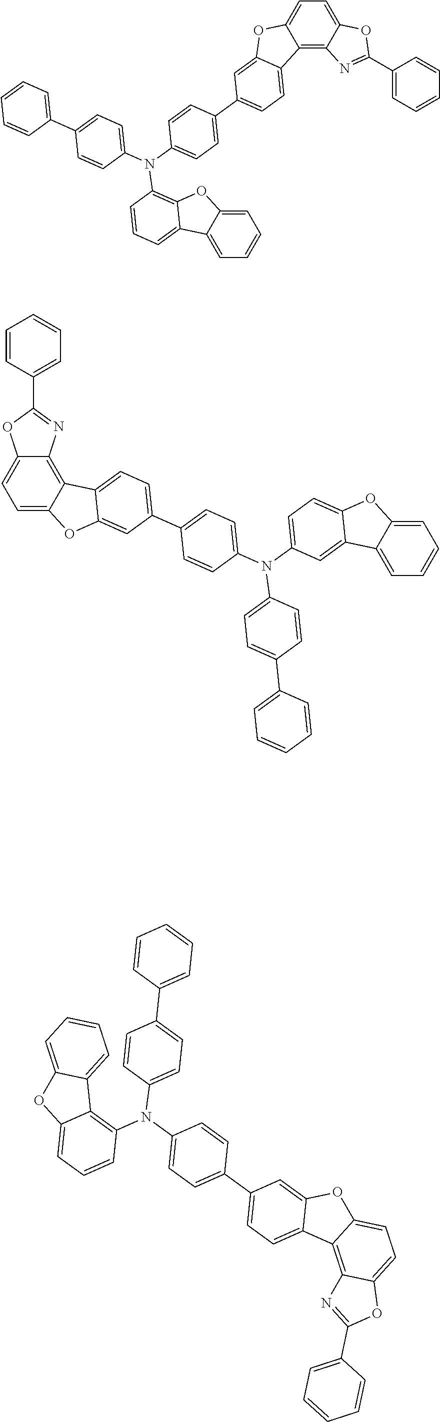 [SMILES: c1ccc(-c2ccc(N(c3ccc(-c4ccc5c(c4)oc4ccc6oc(-c7ccccc7)nc6c45)cc3)c3cccc4c3oc3ccccc34)cc2)cc1 ; c1ccc(-c2ccc(N(c3ccc(-c4ccc5c(c4)oc4ccc6oc(-c7ccccc7)nc6c45)cc3)c3ccc4oc5ccccc5c4c3)cc2)cc1 ; c1ccc(-c2ccc(N(c3ccc(-c4ccc5c(c4)oc4ccc6oc(-c7ccccc7)nc6c45)cc3)c3cccc4oc5ccccc5c34)cc2)cc1]

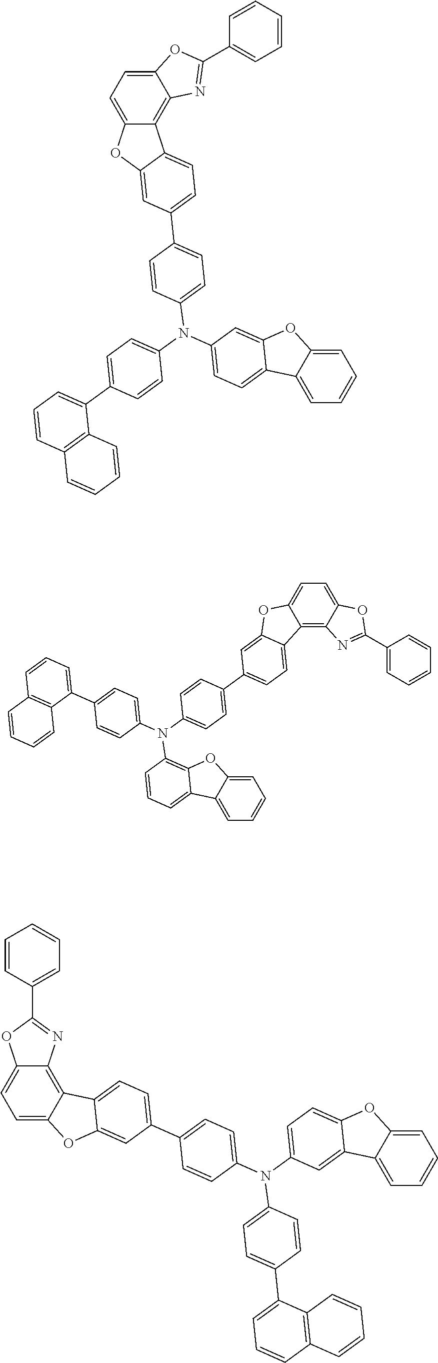 [SMILES: c1ccc(-c2nc3c(ccc4oc5cc(-c6ccc(N(c7ccc(-c8cccc9ccccc89)cc7)c7ccc8c(c7)oc7ccccc78)cc6)ccc5c43)o2)cc1 ; c1ccc(-c2nc3c(ccc4oc5cc(-c6ccc(N(c7ccc(-c8cccc9ccccc89)cc7)c7cccc8c7oc7ccccc78)cc6)ccc5c43)o2)cc1 ; c1ccc(-c2nc3c(ccc4oc5cc(-c6ccc(N(c7ccc(-c8cccc9ccccc89)cc7)c7ccc8oc9ccccc9c8c7)cc6)ccc5c43)o2)cc1]

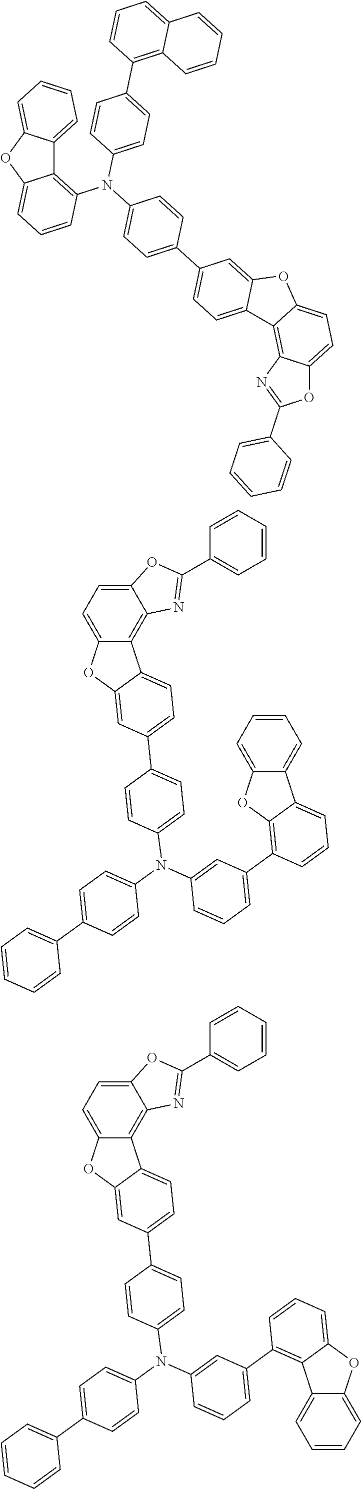

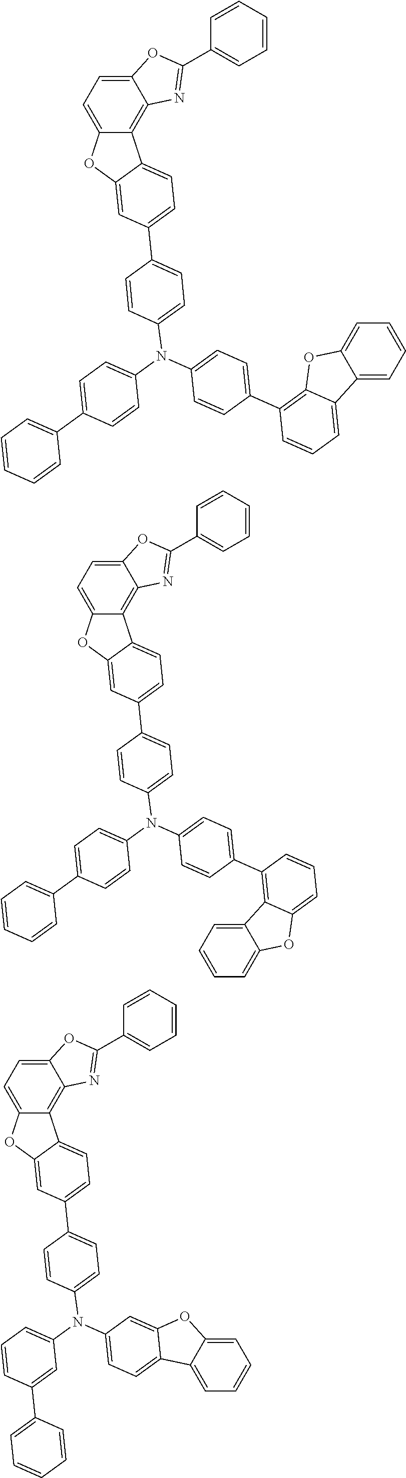 [SMILES: c1ccc(-c2ccc(N(c3ccc(-c4ccc5c(c4)oc4ccc6oc(-c7ccccc7)nc6c45)cc3)c3ccc(-c4cccc5c4oc4ccccc45)cc3)cc2)cc1 ; c1ccc(-c2ccc(N(c3ccc(-c4ccc5c(c4)oc4ccc6oc(-c7ccccc7)nc6c45)cc3)c3ccc(-c4cccc5oc6ccccc6c45)cc3)cc2)cc1 ; c1ccc(-c2cccc(N(c3ccc(-c4ccc5c(c4)oc4ccc6oc(-c7ccccc7)nc6c45)cc3)c3ccc4c(c3)oc3ccccc34)c2)cc1]

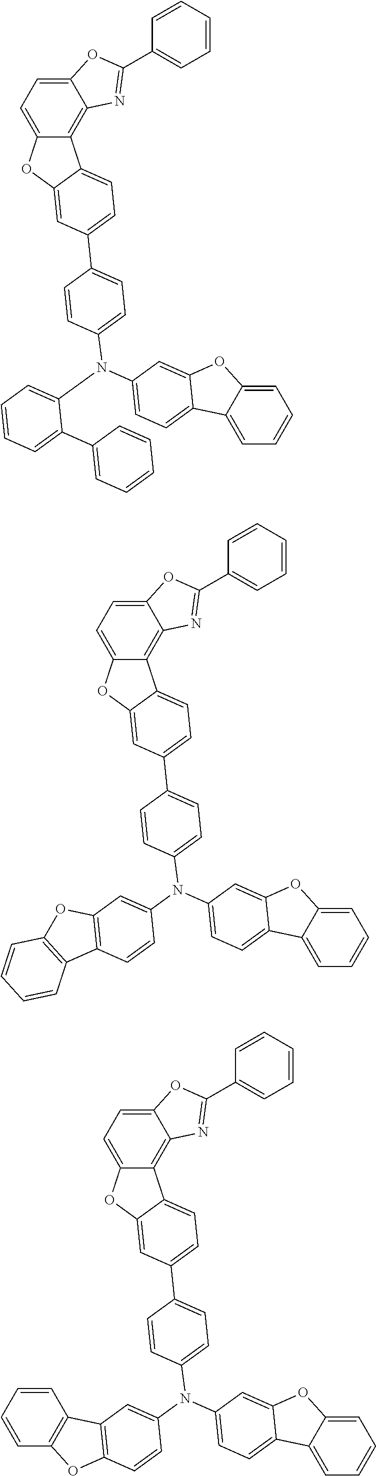 [SMILES: c1ccc(-c2nc3c(ccc4oc5cc(-c6ccc(N(c7ccc8c(c7)oc7ccccc78)c7ccccc7-c7ccccc7)cc6)ccc5c43)o2)cc1 ; c1ccc(-c2nc3c(ccc4oc5cc(-c6ccc(N(c7ccc8c(c7)oc7ccccc78)c7ccc8c(c7)oc7ccccc78)cc6)ccc5c43)o2)cc1 ; c1ccc(-c2nc3c(ccc4oc5cc(-c6ccc(N(c7ccc8c(c7)oc7ccccc78)c7ccc8oc9ccccc9c8c7)cc6)ccc5c43)o2)cc1]

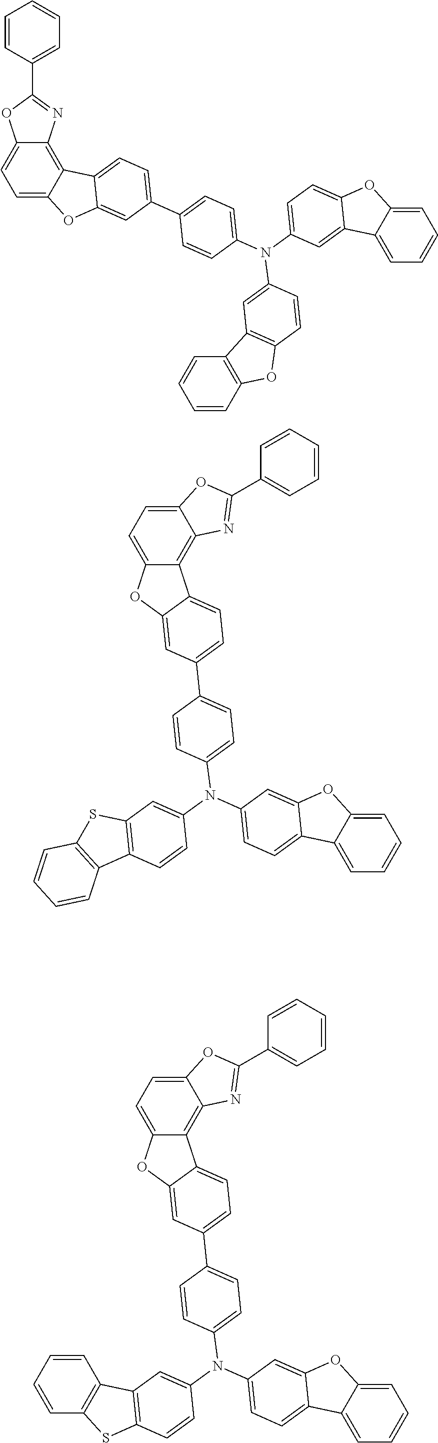 [SMILES: c1ccc(-c2nc3c(ccc4oc5cc(-c6ccc(N(c7ccc8oc9ccccc9c8c7)c7ccc8oc9ccccc9c8c7)cc6)ccc5c43)o2)cc1 ; c1ccc(-c2nc3c(ccc4oc5cc(-c6ccc(N(c7ccc8c(c7)oc7ccccc78)c7ccc8c(c7)sc7ccccc78)cc6)ccc5c43)o2)cc1 ; c1ccc(-c2nc3c(ccc4oc5cc(-c6ccc(N(c7ccc8c(c7)oc7ccccc78)c7ccc8sc9ccccc9c8c7)cc6)ccc5c43)o2)cc1]

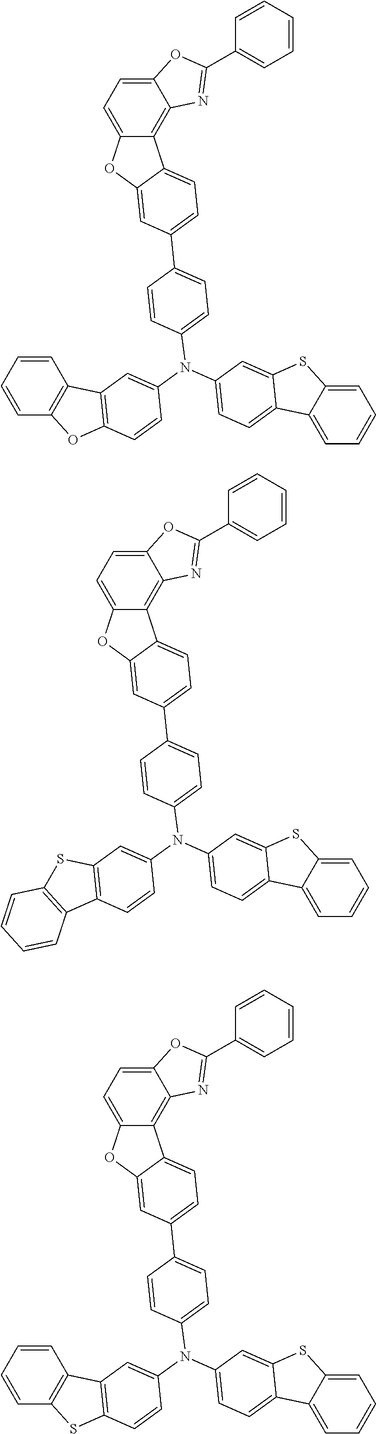 [SMILES: c1ccc(-c2nc3c(ccc4oc5cc(-c6ccc(N(c7ccc8c(c7)sc7ccccc78)c7ccc8oc9ccccc9c8c7)cc6)ccc5c43)o2)cc1 ; c1ccc(-c2nc3c(ccc4oc5cc(-c6ccc(N(c7ccc8c(c7)sc7ccccc78)c7ccc8c(c7)sc7ccccc78)cc6)ccc5c43)o2)cc1 ; c1ccc(-c2nc3c(ccc4oc5cc(-c6ccc(N(c7ccc8c(c7)sc7ccccc78)c7ccc8sc9ccccc9c8c7)cc6)ccc5c43)o2)cc1]

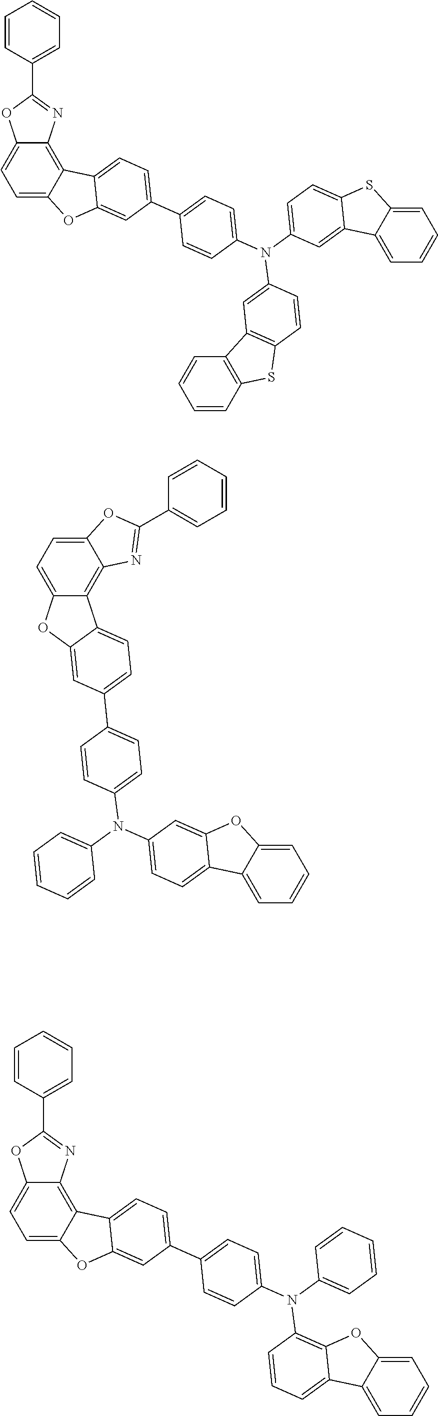 [SMILES: c1ccc(-c2nc3c(ccc4oc5cc(-c6ccc(N(c7ccc8sc9ccccc9c8c7)c7ccc8sc9ccccc9c8c7)cc6)ccc5c43)o2)cc1 ; c1ccc(-c2nc3c(ccc4oc5cc(-c6ccc(N(c7ccccc7)c7ccc8c(c7)oc7ccccc78)cc6)ccc5c43)o2)cc1 ; c1ccc(-c2nc3c(ccc4oc5cc(-c6ccc(N(c7ccccc7)c7cccc8c7oc7ccccc78)cc6)ccc5c43)o2)cc1]

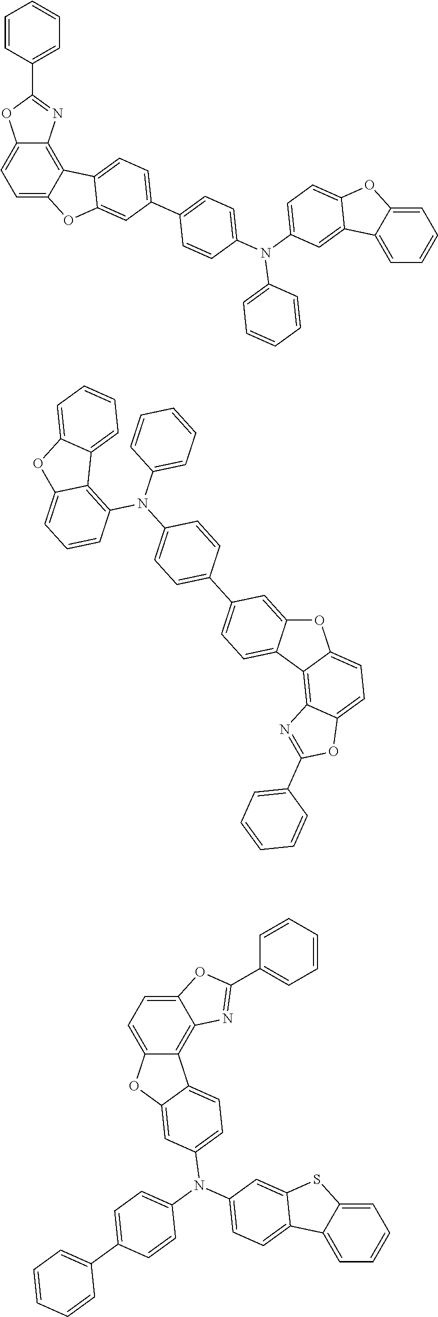 [SMILES: c1ccc(-c2nc3c(ccc4oc5cc(-c6ccc(N(c7ccccc7)c7ccc8oc9ccccc9c8c7)cc6)ccc5c43)o2)cc1 ; c1ccc(-c2nc3c(ccc4oc5cc(-c6ccc(N(c7ccccc7)c7cccc8oc9ccccc9c78)cc6)ccc5c43)o2)cc1 ; c1ccc(-c2ccc(N(c3ccc4c(c3)oc3ccc5oc(-c6ccccc6)nc5c34)c3ccc4c(c3)sc3ccccc34)cc2)cc1]

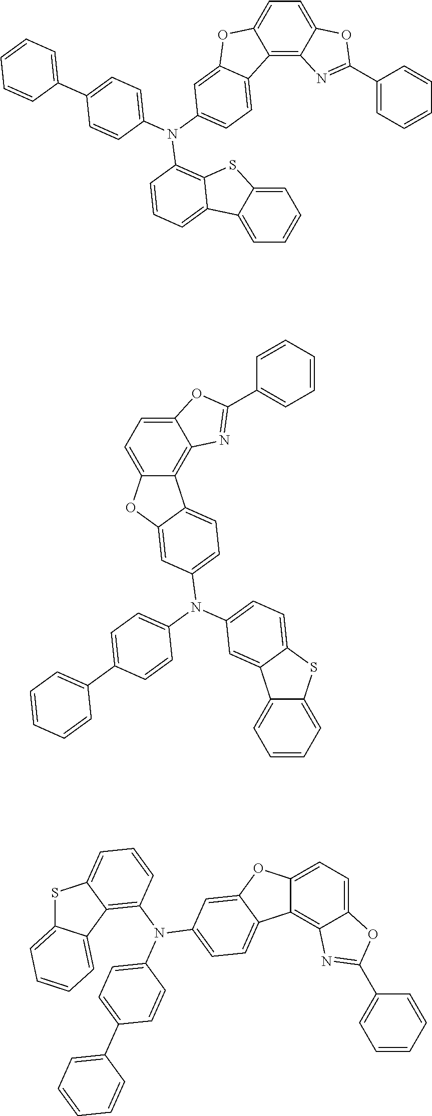 [SMILES: c1ccc(-c2ccc(N(c3ccc4c(c3)oc3ccc5oc(-c6ccccc6)nc5c34)c3cccc4c3sc3ccccc34)cc2)cc1 ; c1ccc(-c2ccc(N(c3ccc4c(c3)oc3ccc5oc(-c6ccccc6)nc5c34)c3ccc4sc5ccccc5c4c3)cc2)cc1 ; c1ccc(-c2ccc(N(c3ccc4c(c3)oc3ccc5oc(-c6ccccc6)nc5c34)c3cccc4sc5ccccc5c34)cc2)cc1]

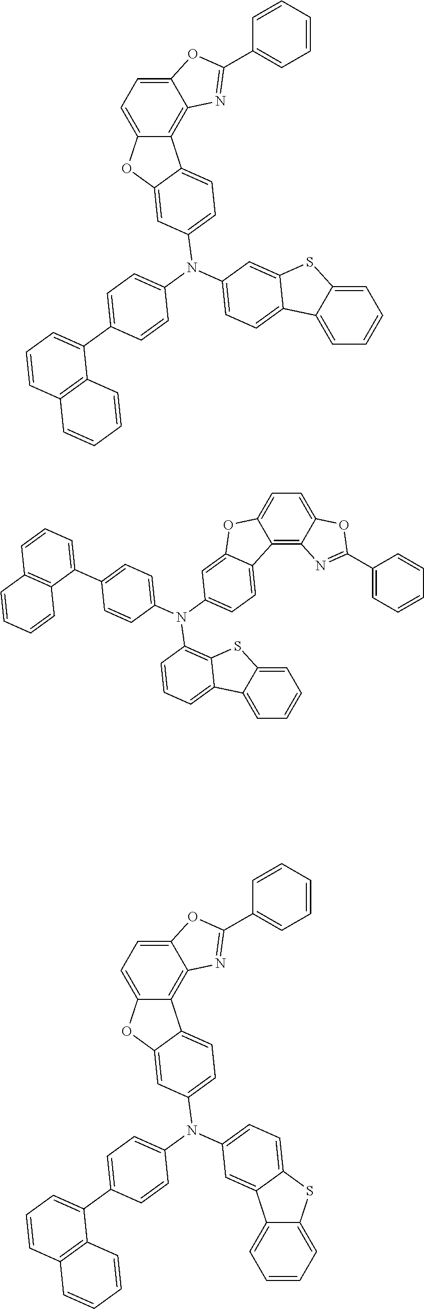 [SMILES: c1ccc(-c2nc3c(ccc4oc5cc(N(c6ccc(-c7cccc8ccccc78)cc6)c6ccc7c(c6)sc6ccccc67)ccc5c43)o2)cc1 ; c1ccc(-c2nc3c(ccc4oc5cc(N(c6ccc(-c7cccc8ccccc78)cc6)c6cccc7c6sc6ccccc67)ccc5c43)o2)cc1 ; c1ccc(-c2nc3c(ccc4oc5cc(N(c6ccc(-c7cccc8ccccc78)cc6)c6ccc7sc8ccccc8c7c6)ccc5c43)o2)cc1]

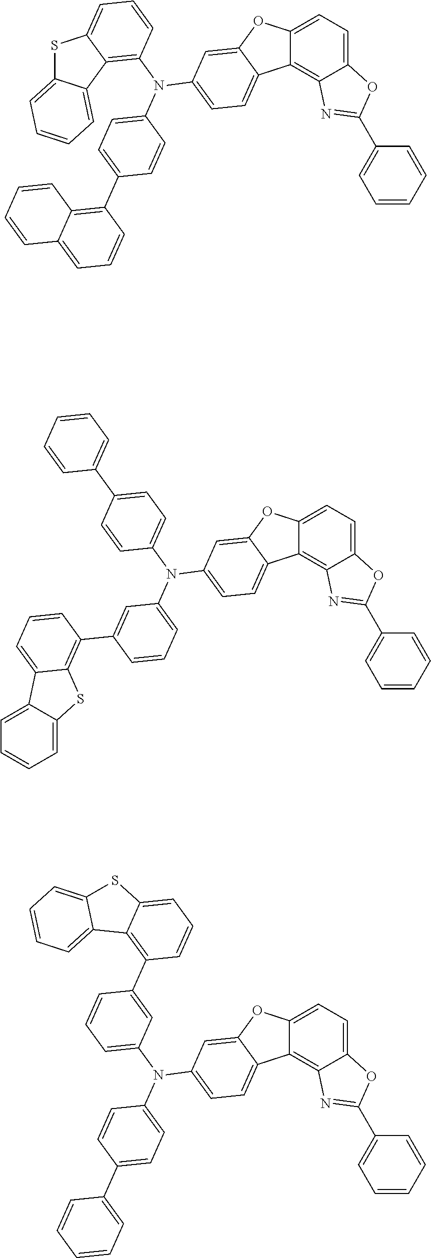 [SMILES: c1ccc(-c2nc3c(ccc4oc5cc(N(c6ccc(-c7cccc8ccccc78)cc6)c6cccc7sc8ccccc8c67)ccc5c43)o2)cc1 ; c1ccc(-c2ccc(N(c3cccc(-c4cccc5c4sc4ccccc45)c3)c3ccc4c(c3)oc3ccc5oc(-c6ccccc6)nc5c34)cc2)cc1 ; c1ccc(-c2ccc(N(c3cccc(-c4cccc5sc6ccccc6c45)c3)c3ccc4c(c3)oc3ccc5oc(-c6ccccc6)nc5c34)cc2)cc1]

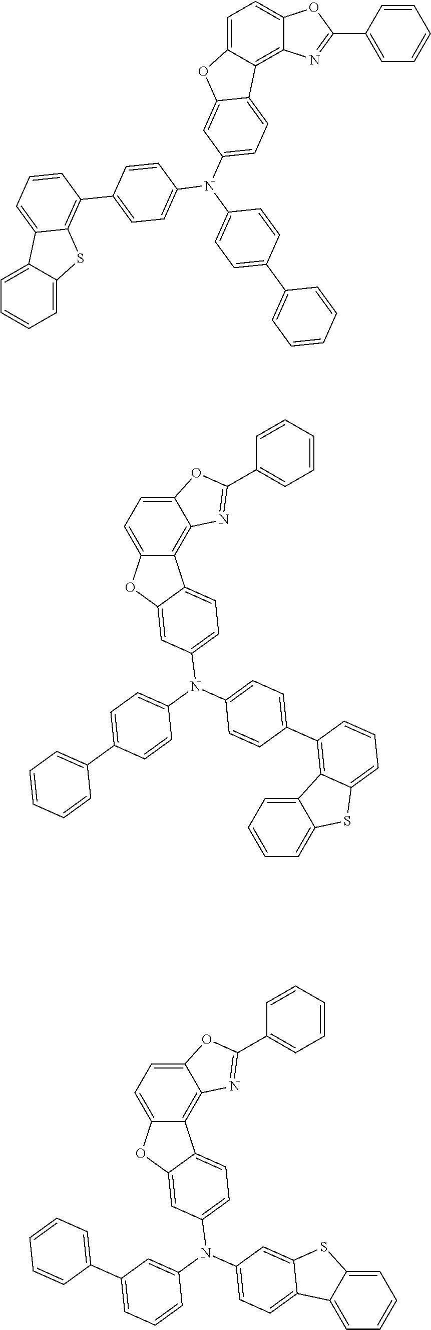 [SMILES: c1ccc(-c2ccc(N(c3ccc(-c4cccc5c4sc4ccccc45)cc3)c3ccc4c(c3)oc3ccc5oc(-c6ccccc6)nc5c34)cc2)cc1 ; c1ccc(-c2ccc(N(c3ccc(-c4cccc5sc6ccccc6c45)cc3)c3ccc4c(c3)oc3ccc5oc(-c6ccccc6)nc5c34)cc2)cc1 ; c1ccc(-c2cccc(N(c3ccc4c(c3)oc3ccc5oc(-c6ccccc6)nc5c34)c3ccc4c(c3)sc3ccccc34)c2)cc1]

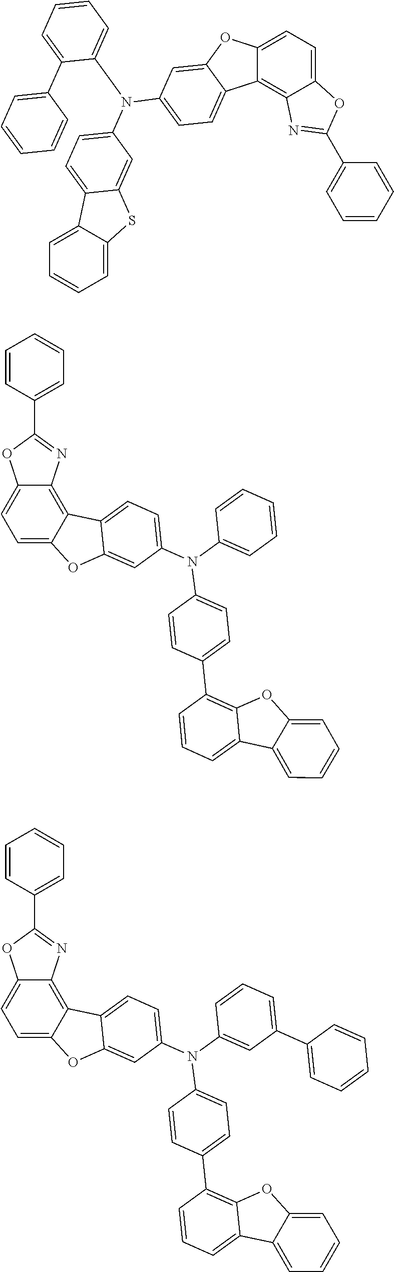 [SMILES: c1ccc(-c2nc3c(ccc4oc5cc(N(c6ccc7c(c6)sc6ccccc67)c6ccccc6-c6ccccc6)ccc5c43)o2)cc1 ; c1ccc(-c2nc3c(ccc4oc5cc(N(c6ccccc6)c6ccc(-c7cccc8c7oc7ccccc78)cc6)ccc5c43)o2)cc1 ; c1ccc(-c2cccc(N(c3ccc(-c4cccc5c4oc4ccccc45)cc3)c3ccc4c(c3)oc3ccc5oc(-c6ccccc6)nc5c34)c2)cc1]

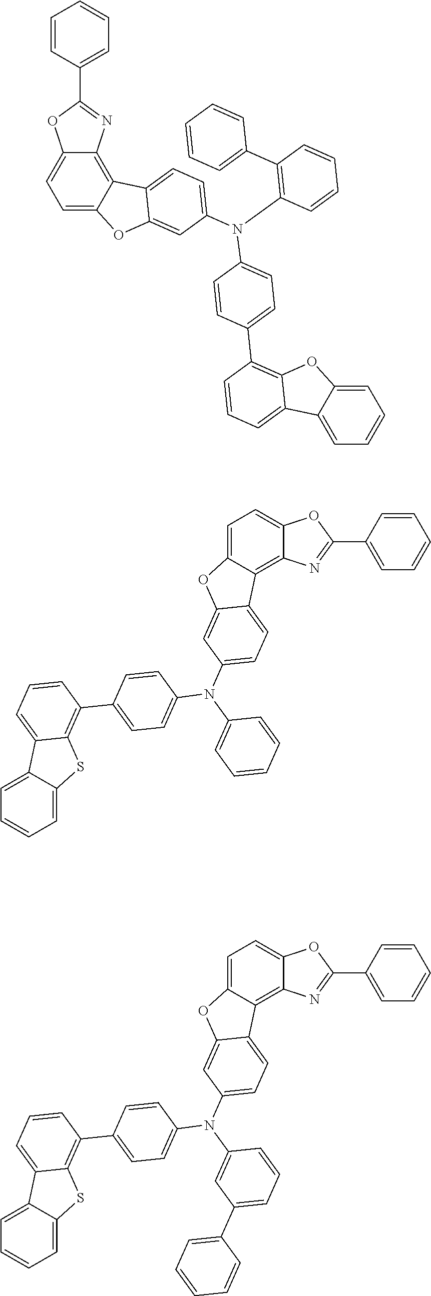 [SMILES: c1ccc(-c2nc3c(ccc4oc5cc(N(c6ccc(-c7cccc8c7oc7ccccc78)cc6)c6ccccc6-c6ccccc6)ccc5c43)o2)cc1 ; c1ccc(-c2nc3c(ccc4oc5cc(N(c6ccccc6)c6ccc(-c7cccc8c7sc7ccccc78)cc6)ccc5c43)o2)cc1 ; c1ccc(-c2cccc(N(c3ccc(-c4cccc5c4sc4ccccc45)cc3)c3ccc4c(c3)oc3ccc5oc(-c6ccccc6)nc5c34)c2)cc1]

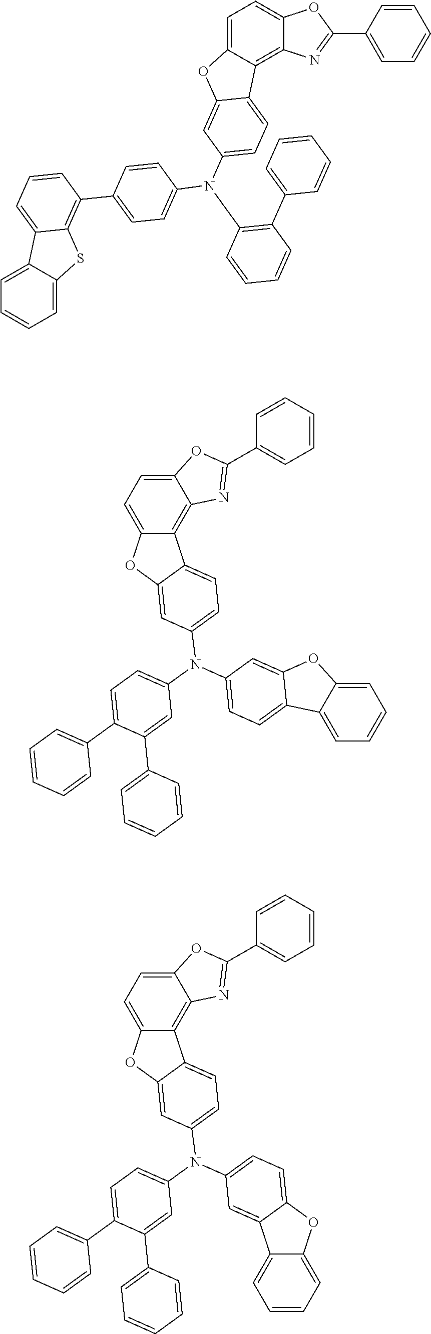 [SMILES: c1ccc(-c2nc3c(ccc4oc5cc(N(c6ccc(-c7cccc8c7sc7ccccc78)cc6)c6ccccc6-c6ccccc6)ccc5c43)o2)cc1 ; c1ccc(-c2nc3c(ccc4oc5cc(N(c6ccc(-c7ccccc7)c(-c7ccccc7)c6)c6ccc7c(c6)oc6ccccc67)ccc5c43)o2)cc1 ; c1ccc(-c2nc3c(ccc4oc5cc(N(c6ccc(-c7ccccc7)c(-c7ccccc7)c6)c6ccc7oc8ccccc8c7c6)ccc5c43)o2)cc1]

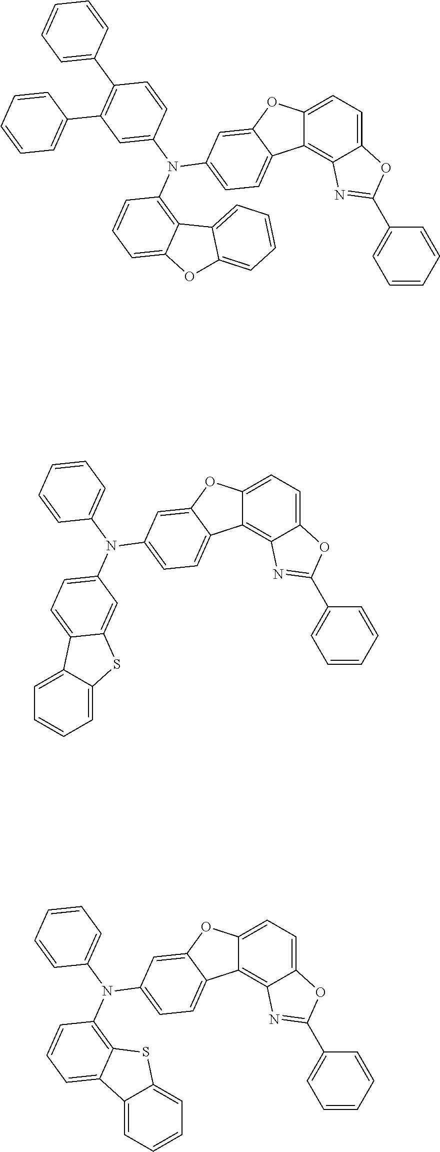 [SMILES: c1ccc(-c2nc3c(ccc4oc5cc(N(c6ccc(-c7ccccc7)c(-c7ccccc7)c6)c6cccc7oc8ccccc8c67)ccc5c43)o2)cc1 ; c1ccc(-c2nc3c(ccc4oc5cc(N(c6ccccc6)c6ccc7c(c6)sc6ccccc67)ccc5c43)o2)cc1 ; c1ccc(-c2nc3c(ccc4oc5cc(N(c6ccccc6)c6cccc7c6sc6ccccc67)ccc5c43)o2)cc1]

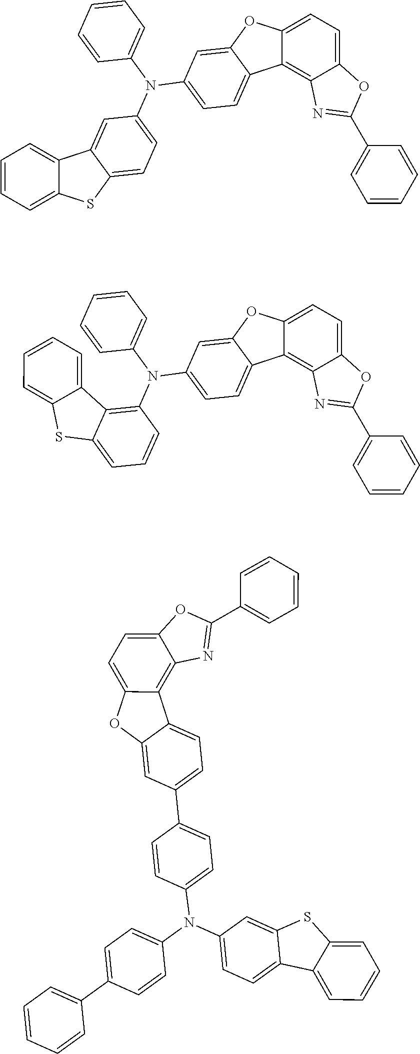 [SMILES: c1ccc(-c2nc3c(ccc4oc5cc(N(c6ccccc6)c6ccc7sc8ccccc8c7c6)ccc5c43)o2)cc1 ; c1ccc(-c2nc3c(ccc4oc5cc(N(c6ccccc6)c6cccc7sc8ccccc8c67)ccc5c43)o2)cc1 ; c1ccc(-c2ccc(N(c3ccc(-c4ccc5c(c4)oc4ccc6oc(-c7ccccc7)nc6c45)cc3)c3ccc4c(c3)sc3ccccc34)cc2)cc1]

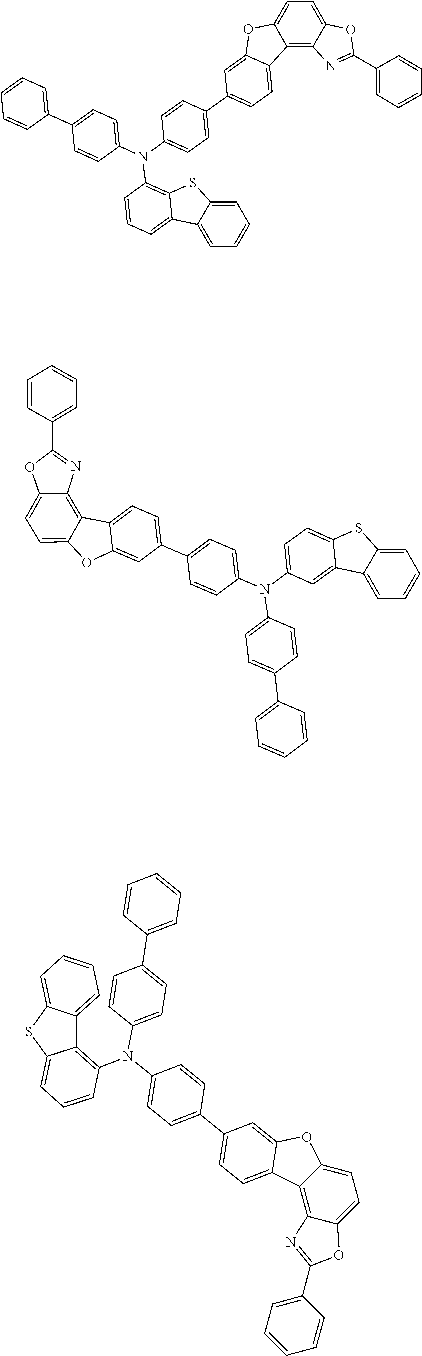 [SMILES: c1ccc(-c2ccc(N(c3ccc(-c4ccc5c(c4)oc4ccc6oc(-c7ccccc7)nc6c45)cc3)c3cccc4c3sc3ccccc34)cc2)cc1 ; c1ccc(-c2ccc(N(c3ccc(-c4ccc5c(c4)oc4ccc6oc(-c7ccccc7)nc6c45)cc3)c3ccc4sc5ccccc5c4c3)cc2)cc1 ; c1ccc(-c2ccc(N(c3ccc(-c4ccc5c(c4)oc4ccc6oc(-c7ccccc7)nc6c45)cc3)c3cccc4sc5ccccc5c34)cc2)cc1]

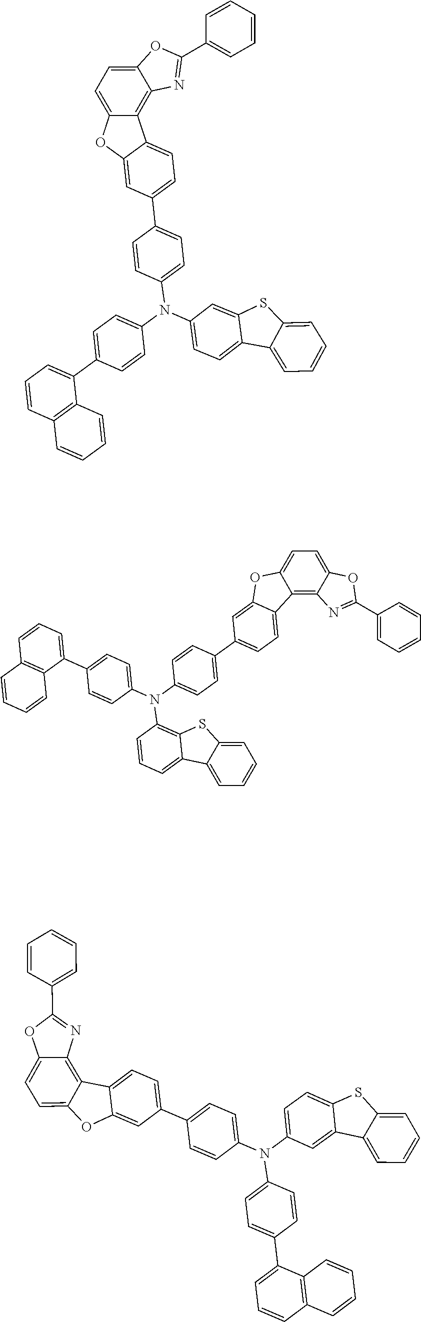 [SMILES: c1ccc(-c2nc3c(ccc4oc5cc(-c6ccc(N(c7ccc(-c8cccc9ccccc89)cc7)c7ccc8c(c7)sc7ccccc78)cc6)ccc5c43)o2)cc1 ; c1ccc(-c2nc3c(ccc4oc5cc(-c6ccc(N(c7ccc(-c8cccc9ccccc89)cc7)c7cccc8c7sc7ccccc78)cc6)ccc5c43)o2)cc1 ; c1ccc(-c2nc3c(ccc4oc5cc(-c6ccc(N(c7ccc(-c8cccc9ccccc89)cc7)c7ccc8sc9ccccc9c8c7)cc6)ccc5c43)o2)cc1]

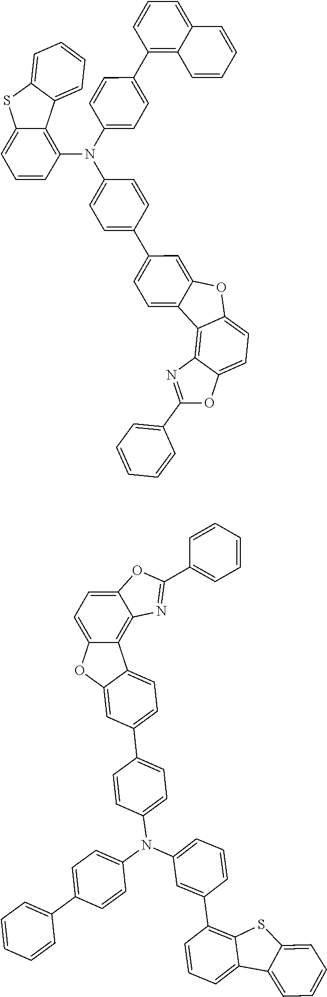 [SMILES: c1ccc(-c2nc3c(ccc4oc5cc(-c6ccc(N(c7ccc(-c8cccc9ccccc89)cc7)c7cccc8sc9ccccc9c78)cc6)ccc5c43)o2)cc1 ; c1ccc(-c2ccc(N(c3ccc(-c4ccc5c(c4)oc4ccc6oc(-c7ccccc7)nc6c45)cc3)c3cccc(-c4cccc5c4sc4ccccc45)c3)cc2)cc1]

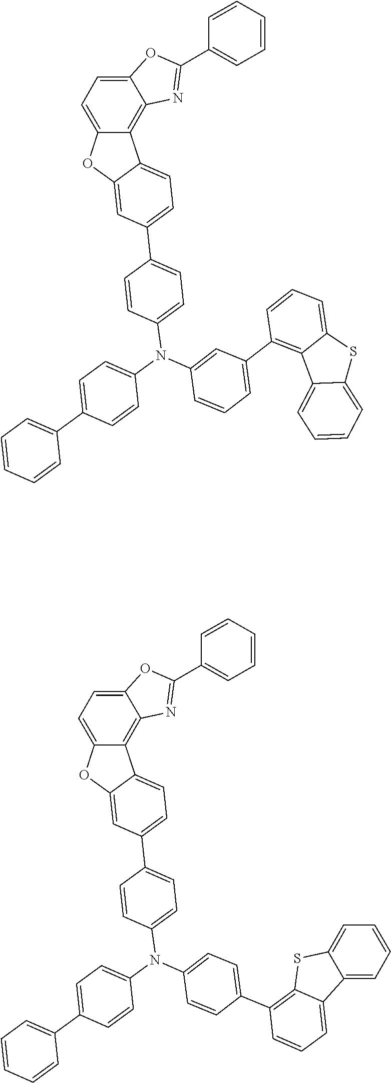 [SMILES: c1ccc(-c2ccc(N(c3ccc(-c4ccc5c(c4)oc4ccc6oc(-c7ccccc7)nc6c45)cc3)c3cccc(-c4cccc5sc6ccccc6c45)c3)cc2)cc1 ; c1ccc(-c2ccc(N(c3ccc(-c4ccc5c(c4)oc4ccc6oc(-c7ccccc7)nc6c45)cc3)c3ccc(-c4cccc5c4sc4ccccc45)cc3)cc2)cc1]

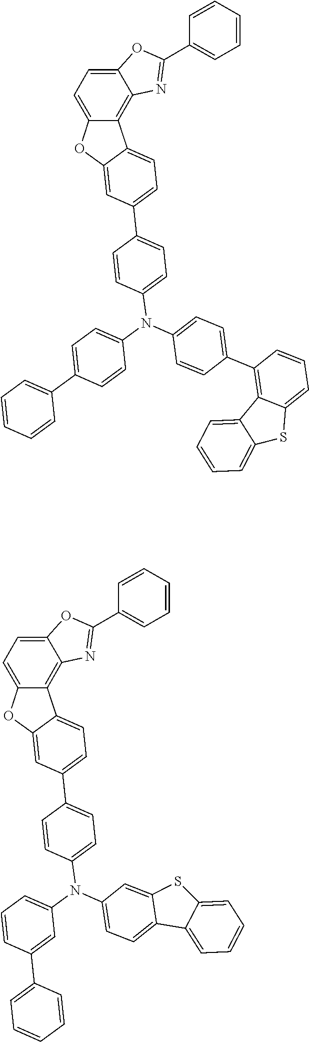 [SMILES: c1ccc(-c2ccc(N(c3ccc(-c4ccc5c(c4)oc4ccc6oc(-c7ccccc7)nc6c45)cc3)c3ccc(-c4cccc5sc6ccccc6c45)cc3)cc2)cc1 ; c1ccc(-c2cccc(N(c3ccc(-c4ccc5c(c4)oc4ccc6oc(-c7ccccc7)nc6c45)cc3)c3ccc4c(c3)sc3ccccc34)c2)cc1]

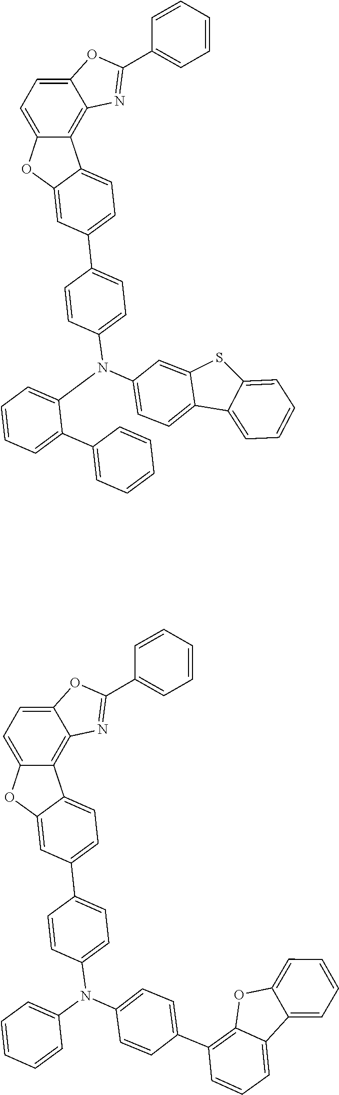 [SMILES: c1ccc(-c2nc3c(ccc4oc5cc(-c6ccc(N(c7ccc8c(c7)sc7ccccc78)c7ccccc7-c7ccccc7)cc6)ccc5c43)o2)cc1 ; c1ccc(-c2nc3c(ccc4oc5cc(-c6ccc(N(c7ccccc7)c7ccc(-c8cccc9c8oc8ccccc89)cc7)cc6)ccc5c43)o2)cc1]

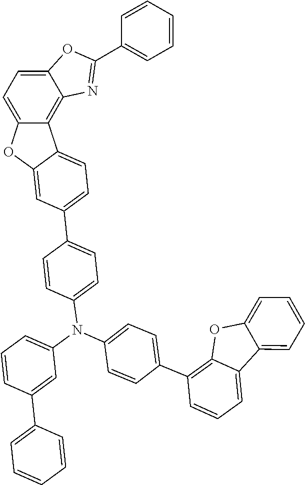 [SMILES: c1ccc(-c2cccc(N(c3ccc(-c4ccc5c(c4)oc4ccc6oc(-c7ccccc7)nc6c45)cc3)c3ccc(-c4cccc5c4oc4ccccc45)cc3)c2)cc1]

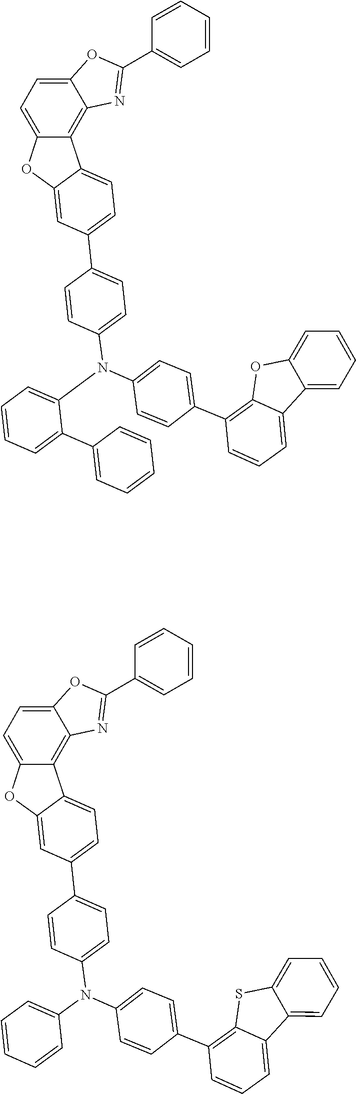 [SMILES: c1ccc(-c2nc3c(ccc4oc5cc(-c6ccc(N(c7ccc(-c8cccc9c8oc8ccccc89)cc7)c7ccccc7-c7ccccc7)cc6)ccc5c43)o2)cc1 ; c1ccc(-c2nc3c(ccc4oc5cc(-c6ccc(N(c7ccccc7)c7ccc(-c8cccc9c8sc8ccccc89)cc7)cc6)ccc5c43)o2)cc1]

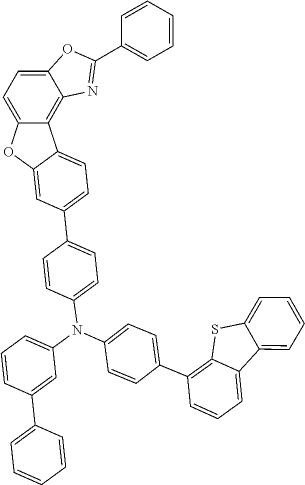 [SMILES: c1ccc(-c2cccc(N(c3ccc(-c4ccc5c(c4)oc4ccc6oc(-c7ccccc7)nc6c45)cc3)c3ccc(-c4cccc5c4sc4ccccc45)cc3)c2)cc1]

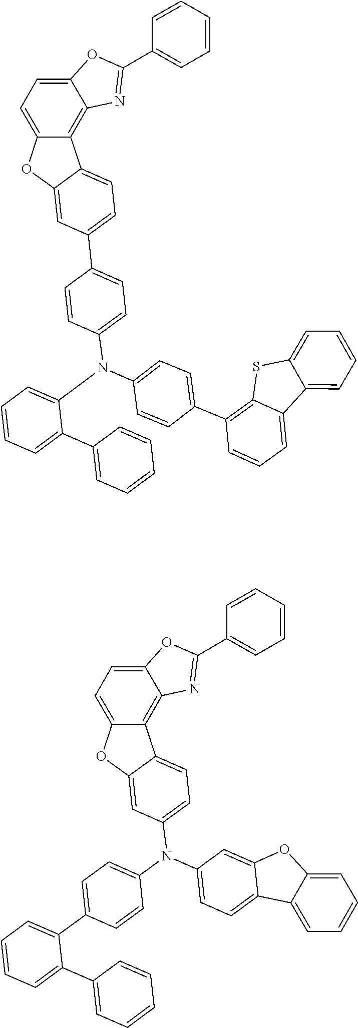 [SMILES: c1ccc(-c2nc3c(ccc4oc5cc(-c6ccc(N(c7ccc(-c8cccc9c8sc8ccccc89)cc7)c7ccccc7-c7ccccc7)cc6)ccc5c43)o2)cc1 ; c1ccc(-c2nc3c(ccc4oc5cc(N(c6ccc(-c7ccccc7-c7ccccc7)cc6)c6ccc7c(c6)oc6ccccc67)ccc5c43)o2)cc1]

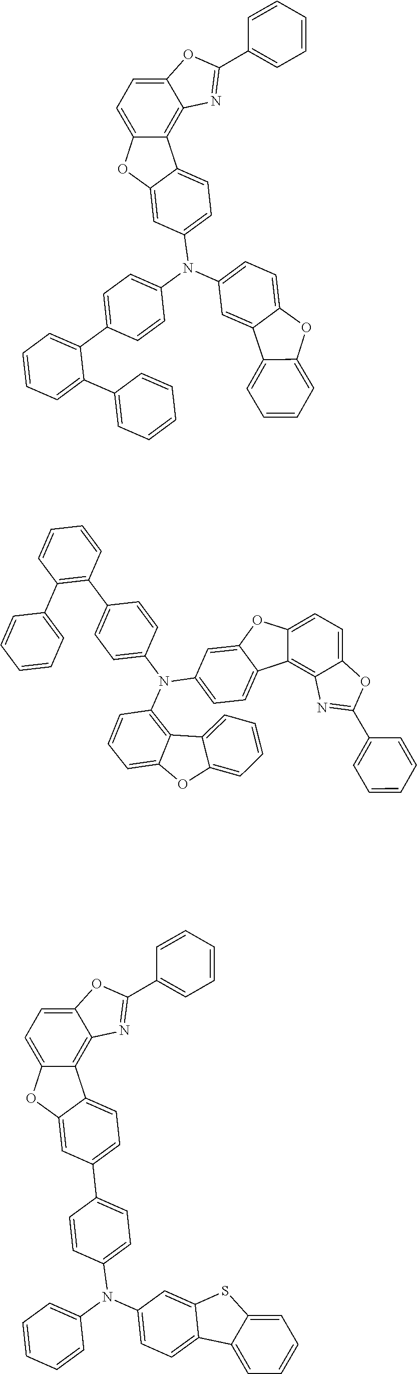 [SMILES: c1ccc(-c2nc3c(ccc4oc5cc(N(c6ccc(-c7ccccc7-c7ccccc7)cc6)c6ccc7oc8ccccc8c7c6)ccc5c43)o2)cc1 ; c1ccc(-c2nc3c(ccc4oc5cc(N(c6ccc(-c7ccccc7-c7ccccc7)cc6)c6cccc7oc8ccccc8c67)ccc5c43)o2)cc1 ; c1ccc(-c2nc3c(ccc4oc5cc(-c6ccc(N(c7ccccc7)c7ccc8c(c7)sc7ccccc78)cc6)ccc5c43)o2)cc1]

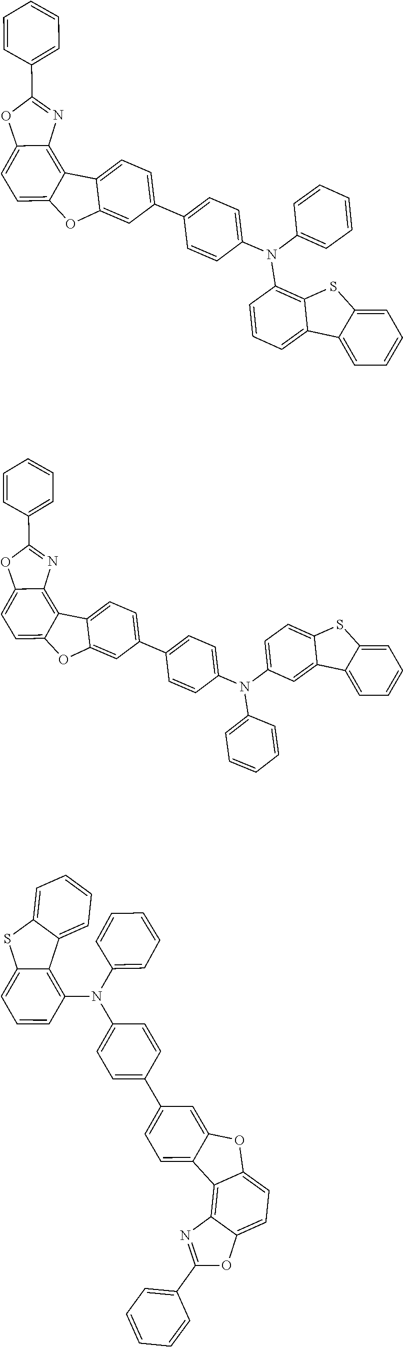 [SMILES: c1ccc(-c2nc3c(ccc4oc5cc(-c6ccc(N(c7ccccc7)c7cccc8c7sc7ccccc78)cc6)ccc5c43)o2)cc1 ; c1ccc(-c2nc3c(ccc4oc5cc(-c6ccc(N(c7ccccc7)c7ccc8sc9ccccc9c8c7)cc6)ccc5c43)o2)cc1 ; c1ccc(-c2nc3c(ccc4oc5cc(-c6ccc(N(c7ccccc7)c7cccc8sc9ccccc9c78)cc6)ccc5c43)o2)cc1]

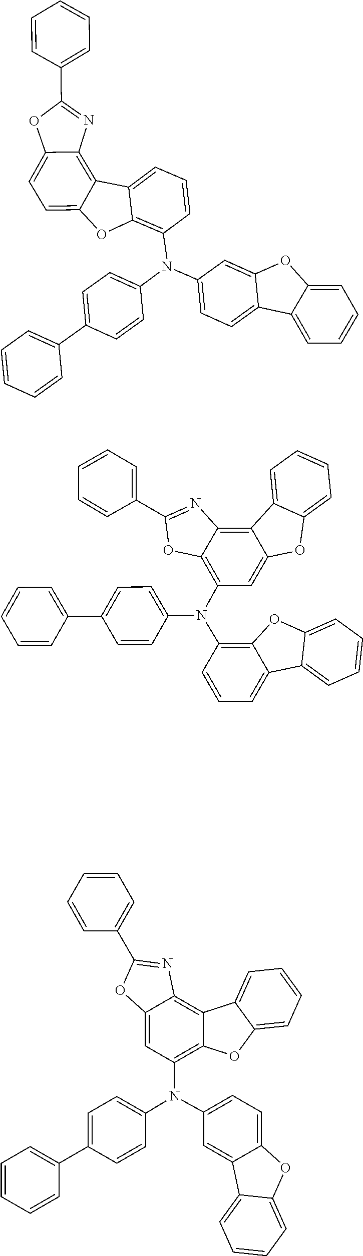 [SMILES: c1ccc(-c2ccc(N(c3ccc4c(c3)oc3ccccc34)c3cccc4c3oc3ccc5oc(-c6ccccc6)nc5c34)cc2)cc1 ; c1ccc(-c2ccc(N(c3cccc4c3oc3ccccc34)c3cc4oc5ccccc5c4c4nc(-c5ccccc5)oc34)cc2)cc1 ; c1ccc(-c2ccc(N(c3ccc4oc5ccccc5c4c3)c3cc4oc(-c5ccccc5)nc4c4c3oc3ccccc34)cc2)cc1]

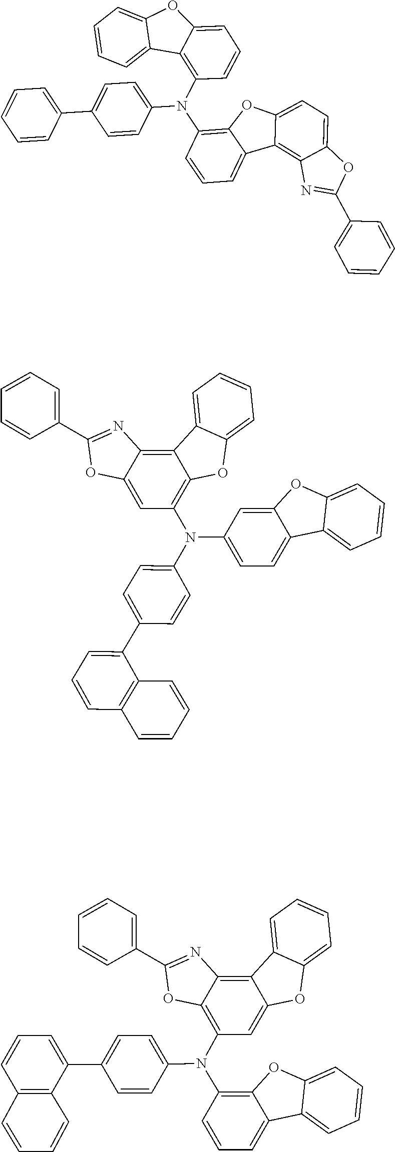 [SMILES: c1ccc(-c2ccc(N(c3cccc4c3oc3ccc5oc(-c6ccccc6)nc5c34)c3cccc4oc5ccccc5c34)cc2)cc1 ; c1ccc(-c2nc3c(cc(N(c4ccc(-c5cccc6ccccc56)cc4)c4ccc5c(c4)oc4ccccc45)c4oc5ccccc5c43)o2)cc1 ; c1ccc(-c2nc3c(o2)c(N(c2ccc(-c4cccc5ccccc45)cc2)c2cccc4c2oc2ccccc24)cc2oc4ccccc4c23)cc1]

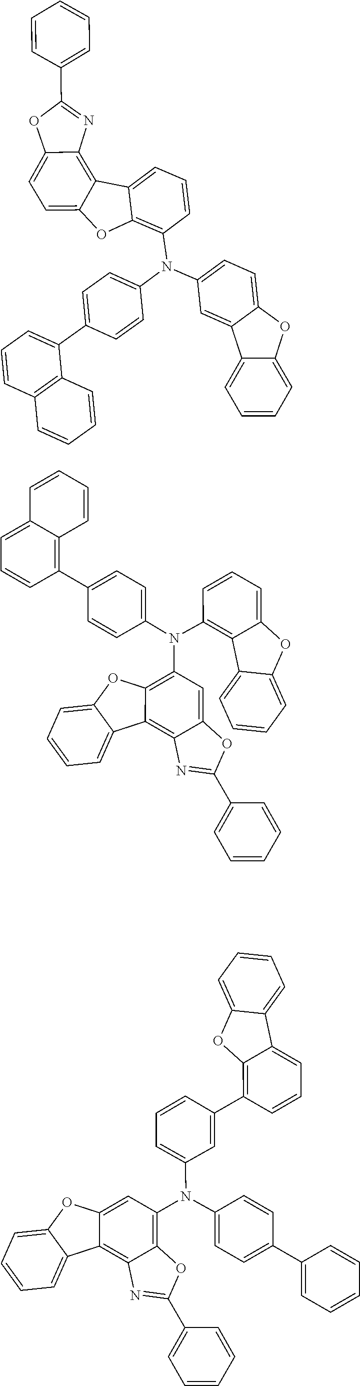

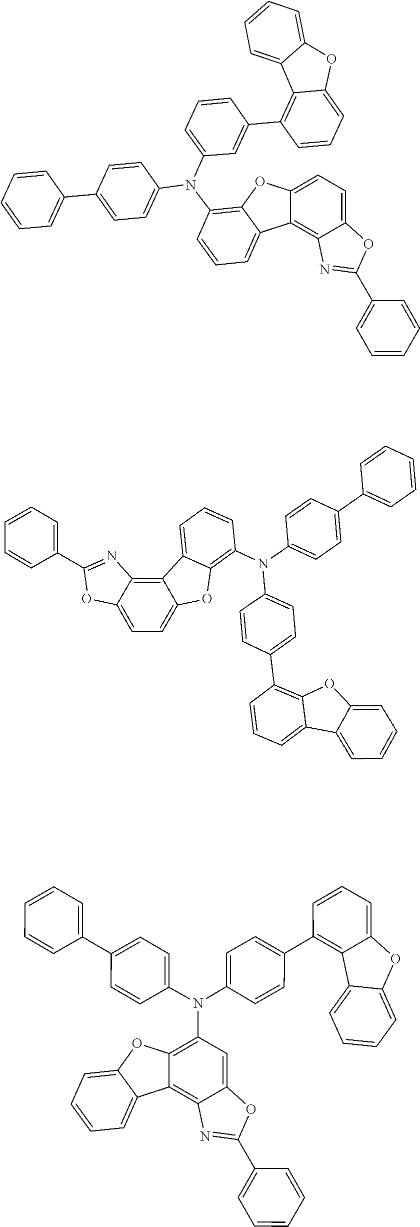 [SMILES: c1ccc(-c2ccc(N(c3cccc(-c4cccc5oc6ccccc6c45)c3)c3cccc4c3oc3ccc5oc(-c6ccccc6)nc5c34)cc2)cc1 ; c1ccc(-c2ccc(N(c3ccc(-c4cccc5c4oc4ccccc45)cc3)c3cccc4c3oc3ccc5oc(-c6ccccc6)nc5c34)cc2)cc1 ; c1ccc(-c2ccc(N(c3ccc(-c4cccc5oc6ccccc6c45)cc3)c3cc4oc(-c5ccccc5)nc4c4c3oc3ccccc34)cc2)cc1]

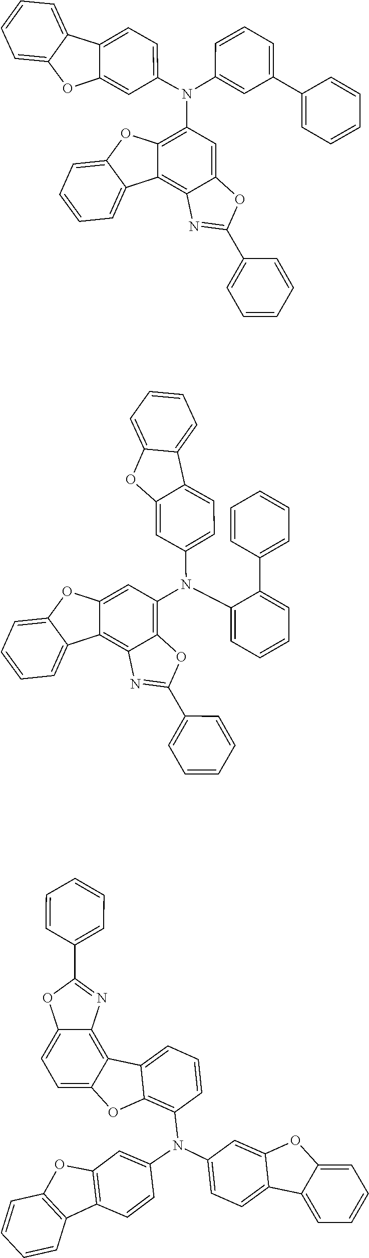 [SMILES: c1ccc(-c2cccc(N(c3ccc4c(c3)oc3ccccc34)c3cc4oc(-c5ccccc5)nc4c4c3oc3ccccc34)c2)cc1 ; c1ccc(-c2nc3c(o2)c(N(c2ccc4c(c2)oc2ccccc24)c2ccccc2-c2ccccc2)cc2oc4ccccc4c23)cc1 ; c1ccc(-c2nc3c(ccc4oc5c(N(c6ccc7c(c6)oc6ccccc67)c6ccc7c(c6)oc6ccccc67)cccc5c43)o2)cc1]

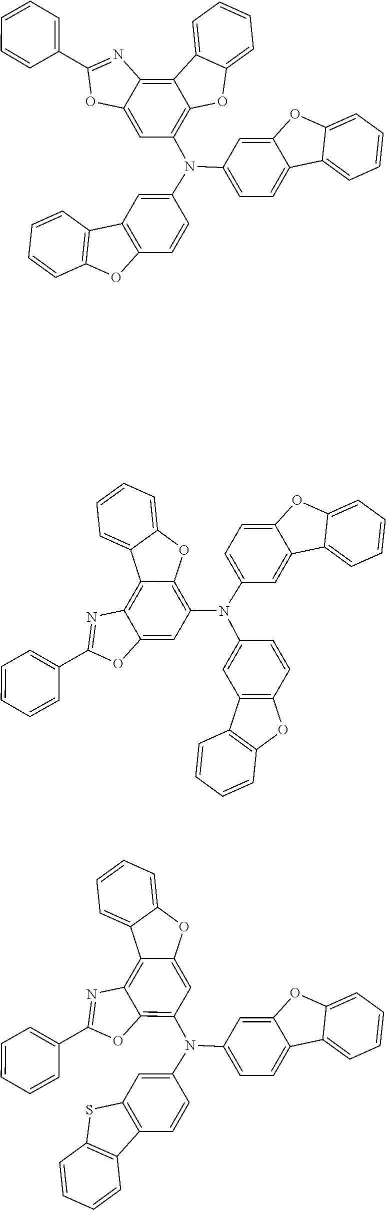 [SMILES: c1ccc(-c2nc3c(cc(N(c4ccc5c(c4)oc4ccccc45)c4ccc5oc6ccccc6c5c4)c4oc5ccccc5c43)o2)cc1 ; c1ccc(-c2nc3c(cc(N(c4ccc5oc6ccccc6c5c4)c4ccc5oc6ccccc6c5c4)c4oc5ccccc5c43)o2)cc1 ; c1ccc(-c2nc3c(o2)c(N(c2ccc4c(c2)oc2ccccc24)c2ccc4c(c2)sc2ccccc24)cc2oc4ccccc4c23)cc1]

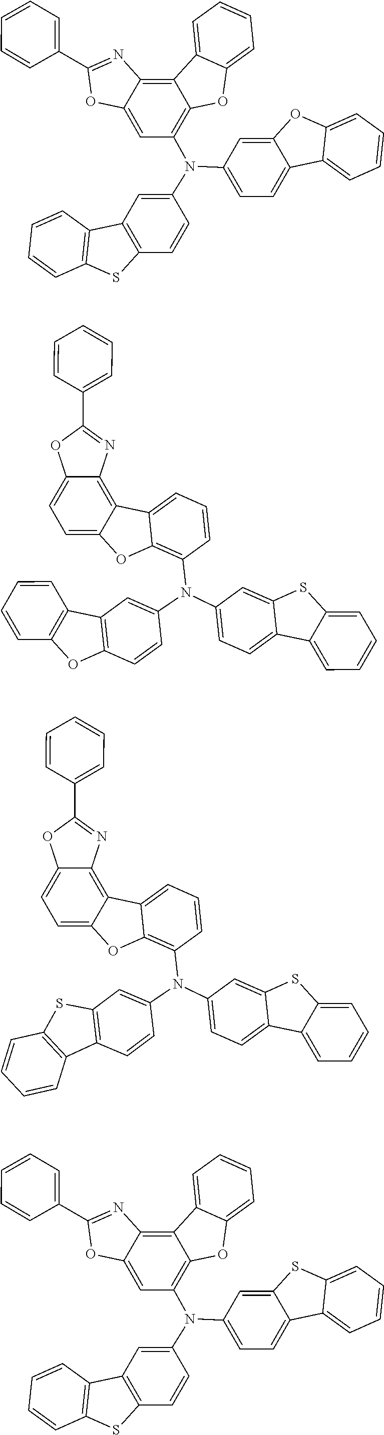 [SMILES: c1ccc(-c2nc3c(cc(N(c4ccc5c(c4)oc4ccccc45)c4ccc5sc6ccccc6c5c4)c4oc5ccccc5c43)o2)cc1 ; c1ccc(-c2nc3c(ccc4oc5c(N(c6ccc7c(c6)sc6ccccc67)c6ccc7oc8ccccc8c7c6)cccc5c43)o2)cc1 ; c1ccc(-c2nc3c(ccc4oc5c(N(c6ccc7c(c6)sc6ccccc67)c6ccc7c(c6)sc6ccccc67)cccc5c43)o2)cc1 ; c1ccc(-c2nc3c(cc(N(c4ccc5c(c4)sc4ccccc45)c4ccc5sc6ccccc6c5c4)c4oc5ccccc5c43)o2)cc1]

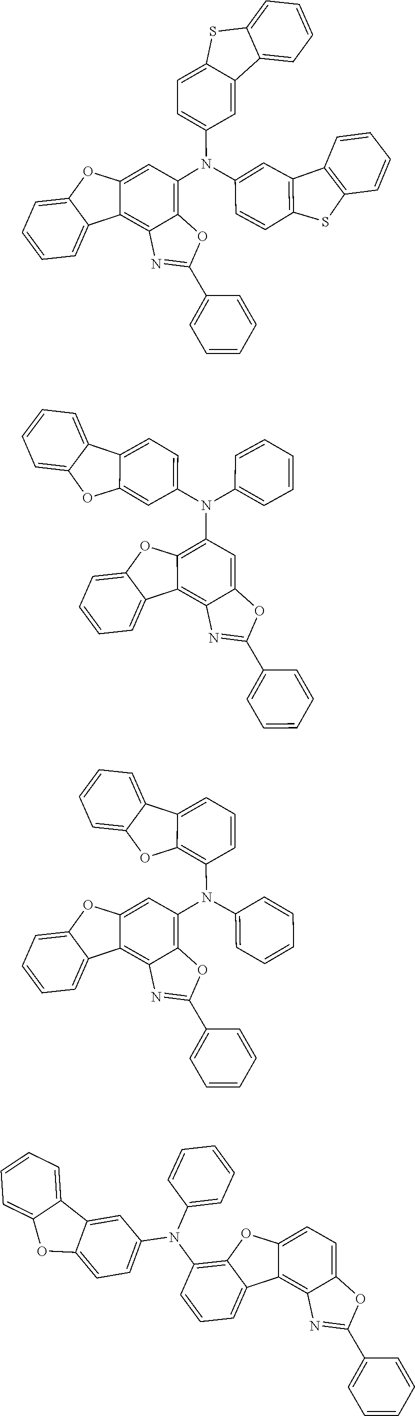 [SMILES: c1ccc(-c2nc3c(o2)c(N(c2ccc4sc5ccccc5c4c2)c2ccc4sc5ccccc5c4c2)cc2oc4ccccc4c23)cc1 ; c1ccc(-c2nc3c(cc(N(c4ccccc4)c4ccc5c(c4)oc4ccccc45)c4oc5ccccc5c43)o2)cc1 ; c1ccc(-c2nc3c(o2)c(N(c2ccccc2)c2cccc4c2oc2ccccc24)cc2oc4ccccc4c23)cc1 ; c1ccc(-c2nc3c(ccc4oc5c(N(c6ccccc6)c6ccc7oc8ccccc8c7c6)cccc5c43)o2)cc1]

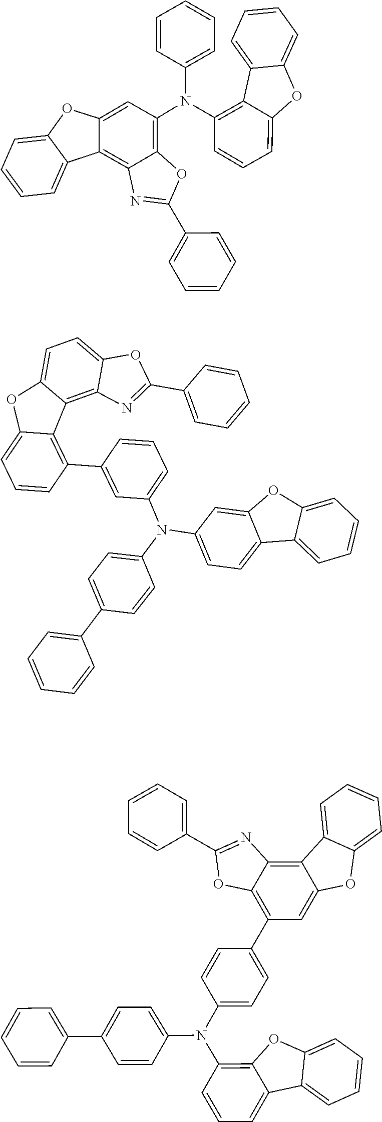 [SMILES: c1ccc(-c2nc3c(o2)c(N(c2ccccc2)c2cccc4oc5ccccc5c24)cc2oc4ccccc4c23)cc1 ; c1ccc(-c2ccc(N(c3cccc(-c4cccc5oc6ccc7oc(-c8ccccc8)nc7c6c45)c3)c3ccc4c(c3)oc3ccccc34)cc2)cc1 ; c1ccc(-c2ccc(N(c3ccc(-c4cc5oc6ccccc6c5c5nc(-c6ccccc6)oc45)cc3)c3cccc4c3oc3ccccc34)cc2)cc1]

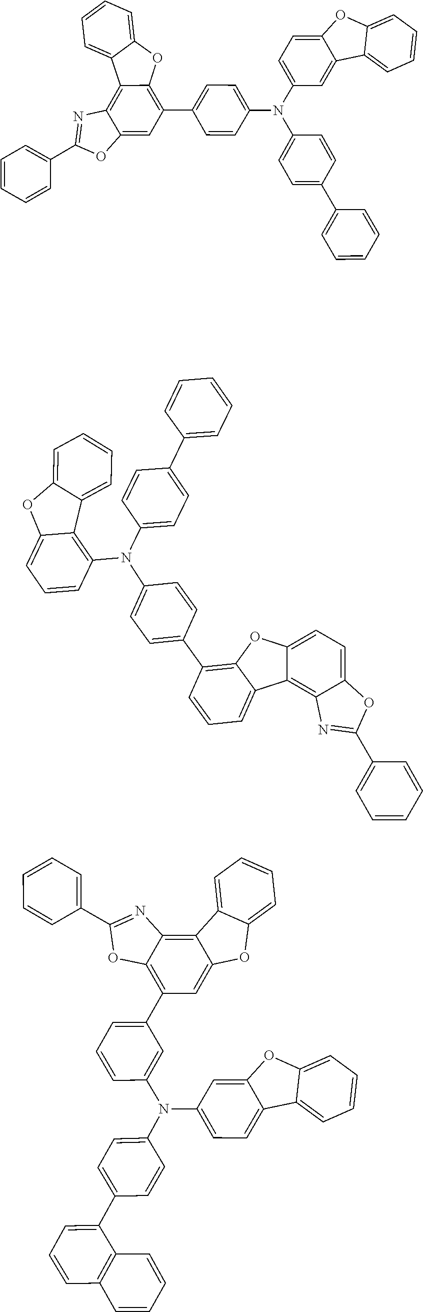 [SMILES: c1ccc(-c2ccc(N(c3ccc(-c4cc5oc(-c6ccccc6)nc5c5c4oc4ccccc45)cc3)c3ccc4oc5ccccc5c4c3)cc2)cc1 ; c1ccc(-c2ccc(N(c3ccc(-c4cccc5c4oc4ccc6oc(-c7ccccc7)nc6c45)cc3)c3cccc4oc5ccccc5c34)cc2)cc1 ; c1ccc(-c2nc3c(o2)c(-c2cccc(N(c4ccc(-c5cccc6ccccc56)cc4)c4ccc5c(c4)oc4ccccc45)c2)cc2oc4ccccc4c23)cc1]

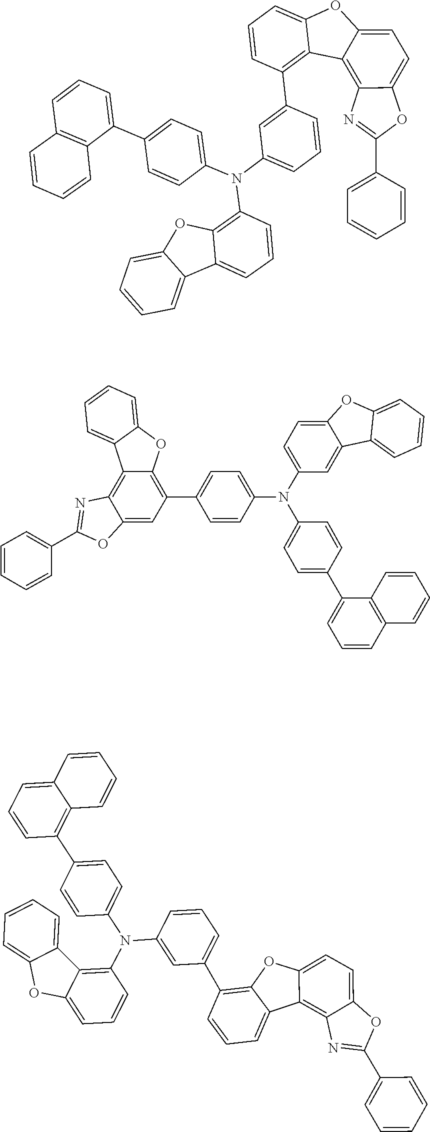 [SMILES: c1ccc(-c2nc3c(ccc4oc5cccc(-c6cccc(N(c7ccc(-c8cccc9ccccc89)cc7)c7cccc8c7oc7ccccc78)c6)c5c43)o2)cc1 ; c1ccc(-c2nc3c(cc(-c4ccc(N(c5ccc(-c6cccc7ccccc67)cc5)c5ccc6oc7ccccc7c6c5)cc4)c4oc5ccccc5c43)o2)cc1 ; c1ccc(-c2nc3c(ccc4oc5c(-c6cccc(N(c7ccc(-c8cccc9ccccc89)cc7)c7cccc8oc9ccccc9c78)c6)cccc5c43)o2)cc1]

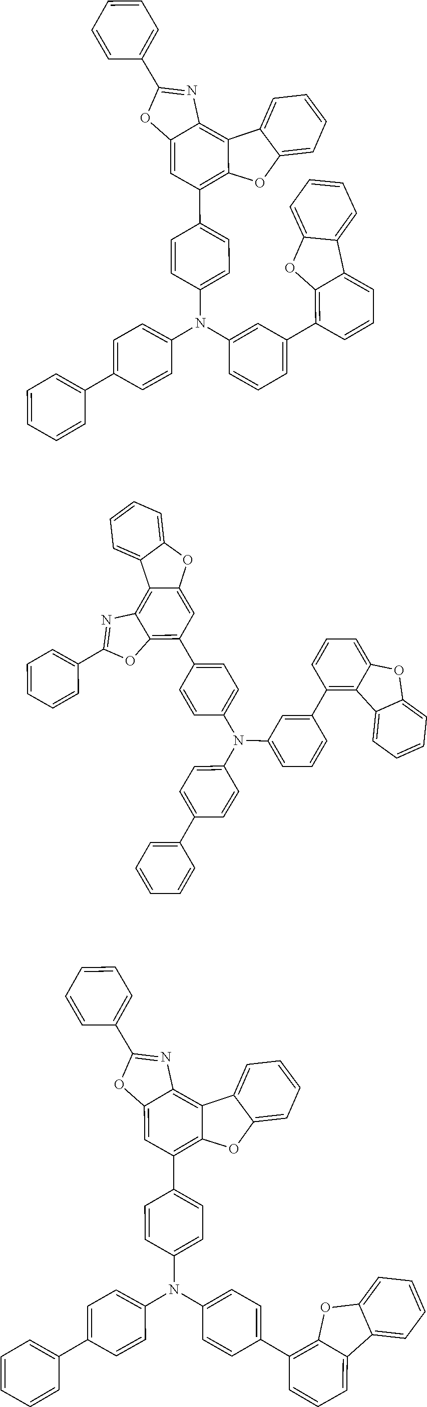 [SMILES: c1ccc(-c2ccc(N(c3ccc(-c4cc5oc(-c6ccccc6)nc5c5c4oc4ccccc45)cc3)c3cccc(-c4cccc5c4oc4ccccc45)c3)cc2)cc1 ; c1ccc(-c2ccc(N(c3ccc(-c4cc5oc6ccccc6c5c5nc(-c6ccccc6)oc45)cc3)c3cccc(-c4cccc5oc6ccccc6c45)c3)cc2)cc1 ; c1ccc(-c2ccc(N(c3ccc(-c4cccc5c4oc4ccccc45)cc3)c3ccc(-c4cc5oc(-c6ccccc6)nc5c5c4oc4ccccc45)cc3)cc2)cc1]

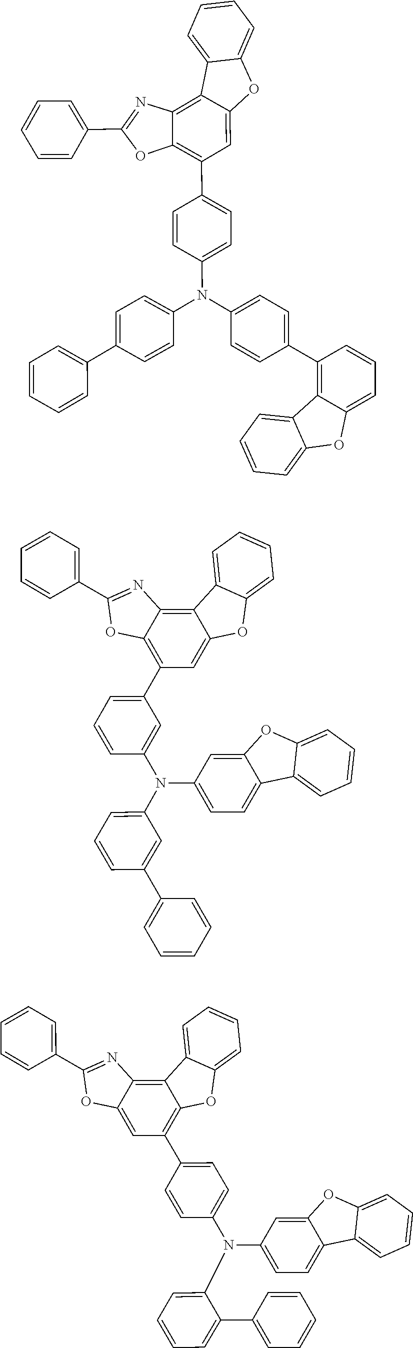 [SMILES: c1ccc(-c2ccc(N(c3ccc(-c4cc5oc6ccccc6c5c5nc(-c6ccccc6)oc45)cc3)c3ccc(-c4cccc5oc6ccccc6c45)cc3)cc2)cc1 ; c1ccc(-c2cccc(N(c3cccc(-c4cc5oc6ccccc6c5c5nc(-c6ccccc6)oc45)c3)c3ccc4c(c3)oc3ccccc34)c2)cc1 ; c1ccc(-c2nc3c(cc(-c4ccc(N(c5ccc6c(c5)oc5ccccc56)c5ccccc5-c5ccccc5)cc4)c4oc5ccccc5c43)o2)cc1]

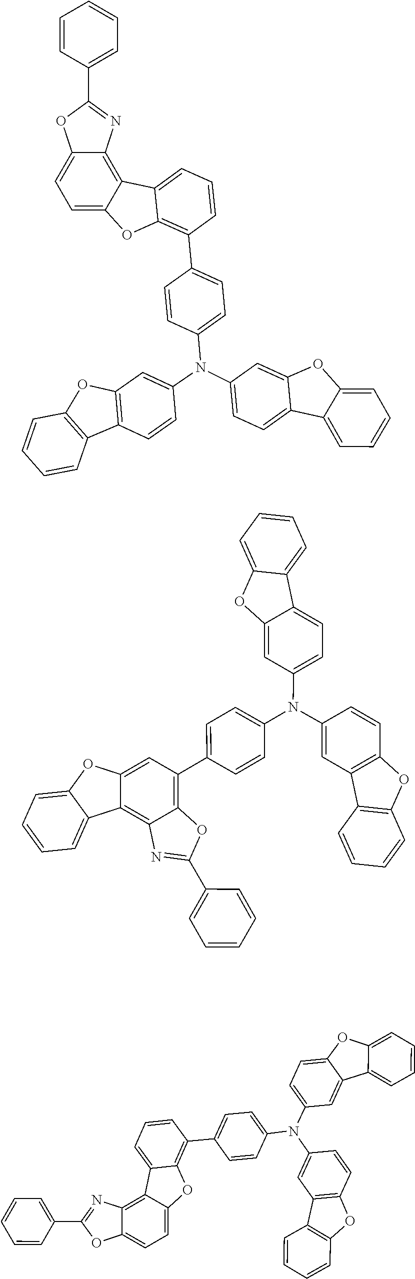 [SMILES: c1ccc(-c2nc3c(ccc4oc5c(-c6ccc(N(c7ccc8c(c7)oc7ccccc78)c7ccc8c(c7)oc7ccccc78)cc6)cccc5c43)o2)cc1 ; c1ccc(-c2nc3c(o2)c(-c2ccc(N(c4ccc5c(c4)oc4ccccc45)c4ccc5oc6ccccc6c5c4)cc2)cc2oc4ccccc4c23)cc1 ; c1ccc(-c2nc3c(ccc4oc5c(-c6ccc(N(c7ccc8oc9ccccc9c8c7)c7ccc8oc9ccccc9c8c7)cc6)cccc5c43)o2)cc1]

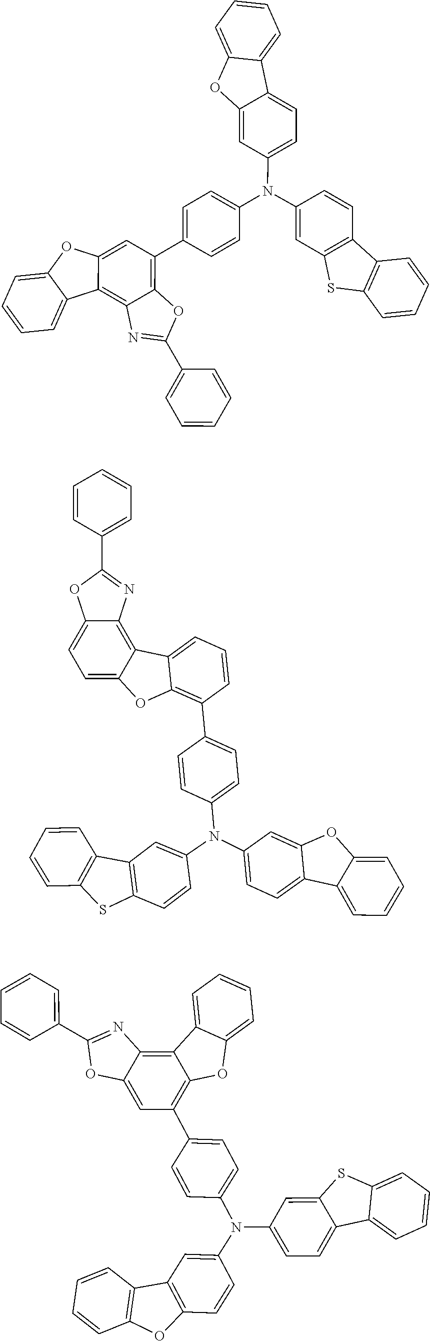 [SMILES: c1ccc(-c2nc3c(o2)c(-c2ccc(N(c4ccc5c(c4)oc4ccccc45)c4ccc5c(c4)sc4ccccc45)cc2)cc2oc4ccccc4c23)cc1 ; c1ccc(-c2nc3c(ccc4oc5c(-c6ccc(N(c7ccc8c(c7)oc7ccccc78)c7ccc8sc9ccccc9c8c7)cc6)cccc5c43)o2)cc1 ; c1ccc(-c2nc3c(cc(-c4ccc(N(c5ccc6c(c5)sc5ccccc56)c5ccc6oc7ccccc7c6c5)cc4)c4oc5ccccc5c43)o2)cc1]

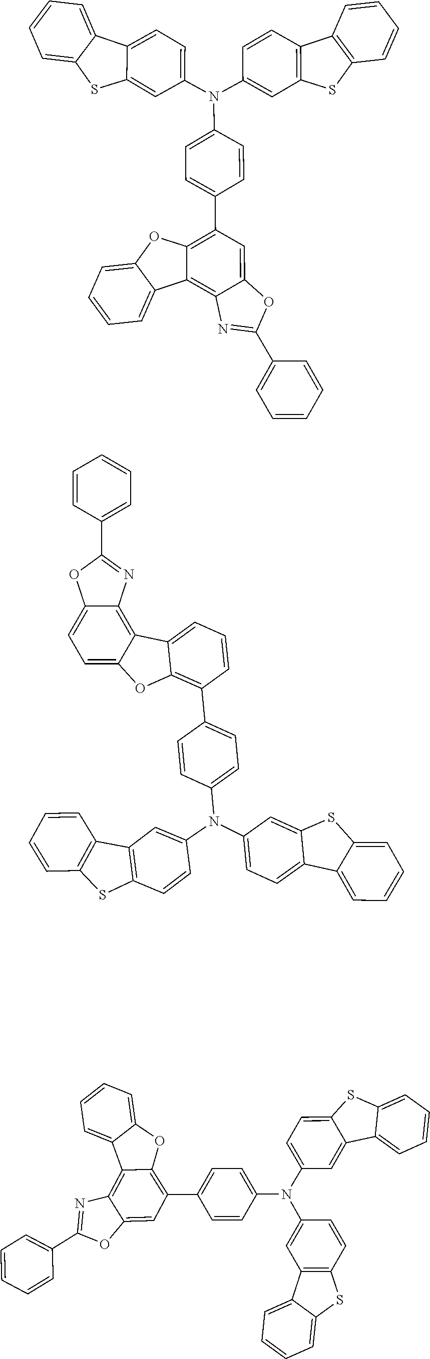 [SMILES: c1ccc(-c2nc3c(cc(-c4ccc(N(c5ccc6c(c5)sc5ccccc56)c5ccc6c(c5)sc5ccccc56)cc4)c4oc5ccccc5c43)o2)cc1 ; c1ccc(-c2nc3c(ccc4oc5c(-c6ccc(N(c7ccc8c(c7)sc7ccccc78)c7ccc8sc9ccccc9c8c7)cc6)cccc5c43)o2)cc1 ; c1ccc(-c2nc3c(cc(-c4ccc(N(c5ccc6sc7ccccc7c6c5)c5ccc6sc7ccccc7c6c5)cc4)c4oc5ccccc5c43)o2)cc1]

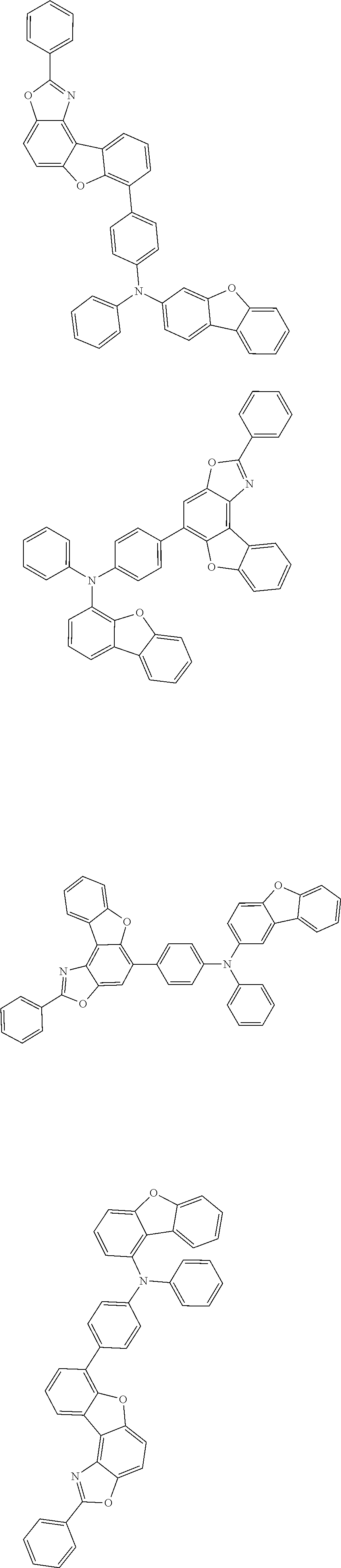 [SMILES: c1ccc(-c2nc3c(ccc4oc5c(-c6ccc(N(c7ccccc7)c7ccc8c(c7)oc7ccccc78)cc6)cccc5c43)o2)cc1 ; c1ccc(-c2nc3c(cc(-c4ccc(N(c5ccccc5)c5cccc6c5oc5ccccc56)cc4)c4oc5ccccc5c43)o2)cc1 ; c1ccc(-c2nc3c(cc(-c4ccc(N(c5ccccc5)c5ccc6oc7ccccc7c6c5)cc4)c4oc5ccccc5c43)o2)cc1 ; c1ccc(-c2nc3c(ccc4oc5c(-c6ccc(N(c7ccccc7)c7cccc8oc9ccccc9c78)cc6)cccc5c43)o2)cc1]

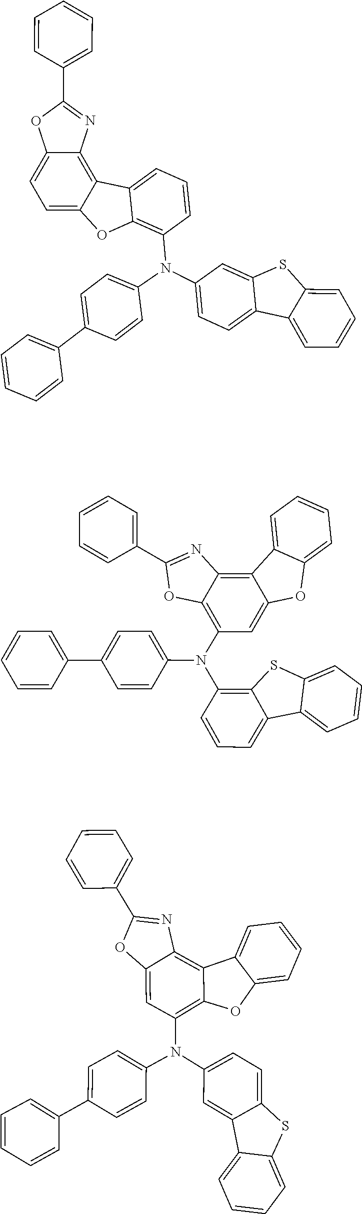 [SMILES: c1ccc(-c2ccc(N(c3ccc4c(c3)sc3ccccc34)c3cccc4c3oc3ccc5oc(-c6ccccc6)nc5c34)cc2)cc1 ; c1ccc(-c2ccc(N(c3cc4oc5ccccc5c4c4nc(-c5ccccc5)oc34)c3cccc4c3sc3ccccc34)cc2)cc1 ; c1ccc(-c2ccc(N(c3ccc4sc5ccccc5c4c3)c3cc4oc(-c5ccccc5)nc4c4c3oc3ccccc34)cc2)cc1]

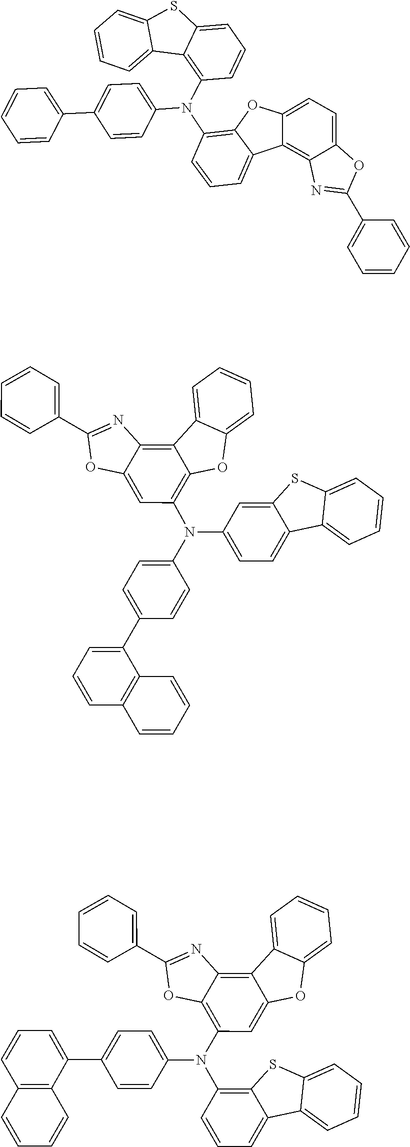 [SMILES: c1ccc(-c2ccc(N(c3cccc4c3oc3ccc5oc(-c6ccccc6)nc5c34)c3cccc4sc5ccccc5c34)cc2)cc1 ; c1ccc(-c2nc3c(cc(N(c4ccc(-c5cccc6ccccc56)cc4)c4ccc5c(c4)sc4ccccc45)c4oc5ccccc5c43)o2)cc1 ; c1ccc(-c2nc3c(o2)c(N(c2ccc(-c4cccc5ccccc45)cc2)c2cccc4c2sc2ccccc24)cc2oc4ccccc4c23)cc1]

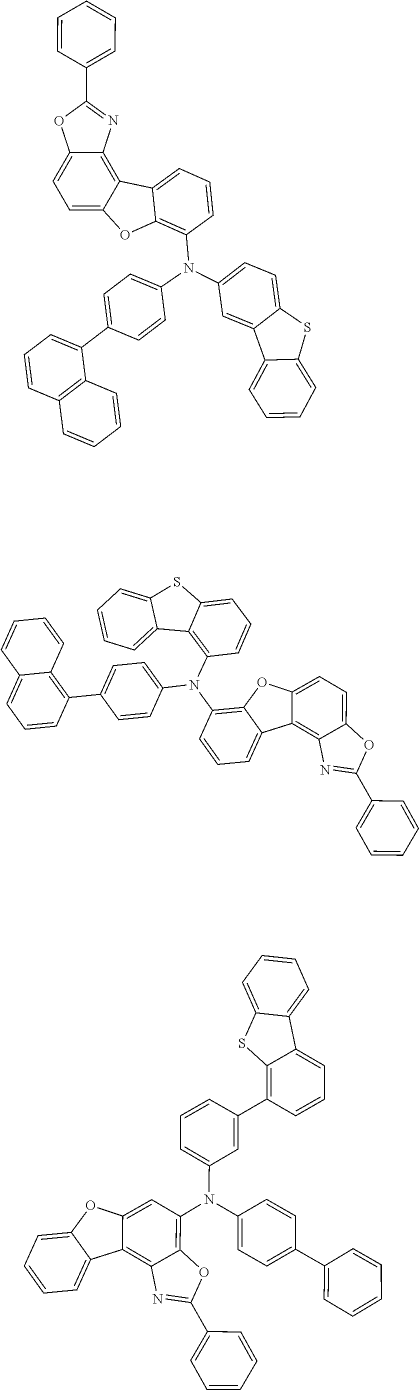 [SMILES: c1ccc(-c2nc3c(ccc4oc5c(N(c6ccc(-c7cccc8ccccc78)cc6)c6ccc7sc8ccccc8c7c6)cccc5c43)o2)cc1 ; c1ccc(-c2nc3c(ccc4oc5c(N(c6ccc(-c7cccc8ccccc78)cc6)c6cccc7sc8ccccc8c67)cccc5c43)o2)cc1 ; c1ccc(-c2ccc(N(c3cccc(-c4cccc5c4sc4ccccc45)c3)c3cc4oc5ccccc5c4c4nc(-c5ccccc5)oc34)cc2)cc1]

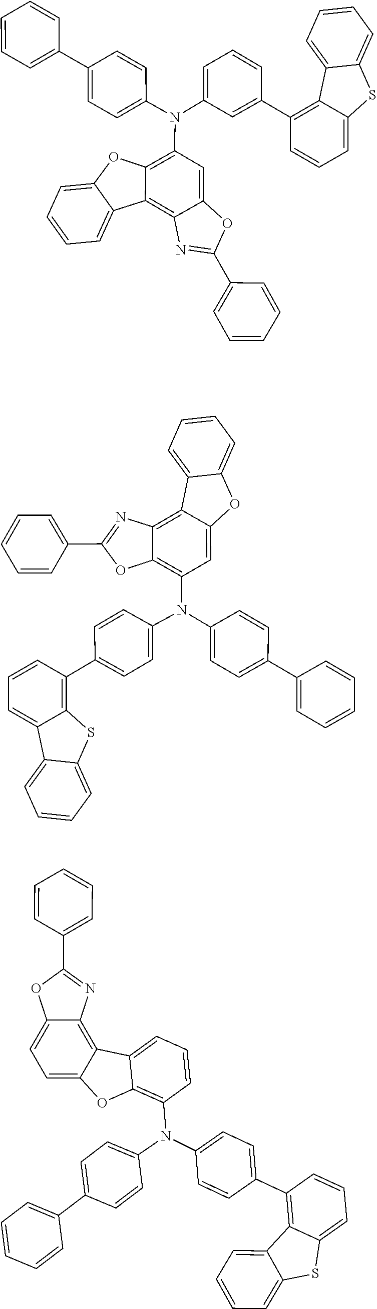 [SMILES: c1ccc(-c2ccc(N(c3cccc(-c4cccc5sc6ccccc6c45)c3)c3cc4oc(-c5ccccc5)nc4c4c3oc3ccccc34)cc2)cc1 ; c1ccc(-c2ccc(N(c3ccc(-c4cccc5c4sc4ccccc45)cc3)c3cc4oc5ccccc5c4c4nc(-c5ccccc5)oc34)cc2)cc1 ; c1ccc(-c2ccc(N(c3ccc(-c4cccc5sc6ccccc6c45)cc3)c3cccc4c3oc3ccc5oc(-c6ccccc6)nc5c34)cc2)cc1]

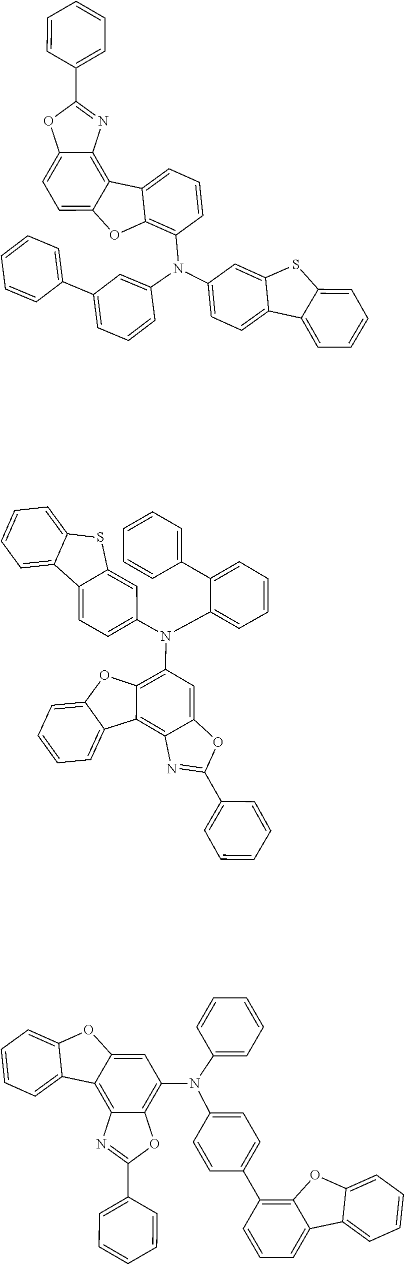 [SMILES: c1ccc(-c2cccc(N(c3ccc4c(c3)sc3ccccc34)c3cccc4c3oc3ccc5oc(-c6ccccc6)nc5c34)c2)cc1 ; c1ccc(-c2nc3c(cc(N(c4ccc5c(c4)sc4ccccc45)c4ccccc4-c4ccccc4)c4oc5ccccc5c43)o2)cc1 ; c1ccc(-c2nc3c(o2)c(N(c2ccccc2)c2ccc(-c4cccc5c4oc4ccccc45)cc2)cc2oc4ccccc4c23)cc1]

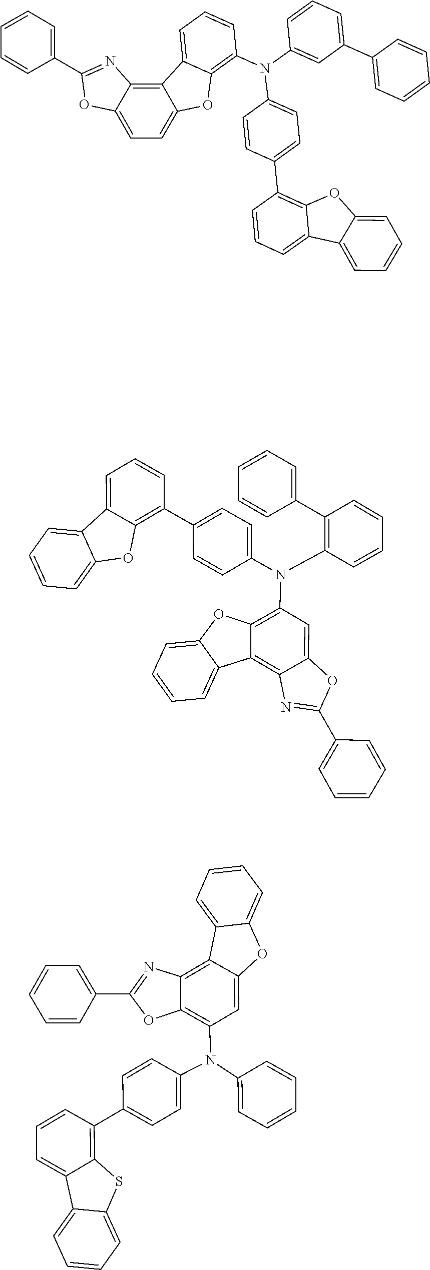 [SMILES: c1ccc(-c2cccc(N(c3ccc(-c4cccc5c4oc4ccccc45)cc3)c3cccc4c3oc3ccc5oc(-c6ccccc6)nc5c34)c2)cc1 ; c1ccc(-c2nc3c(cc(N(c4ccc(-c5cccc6c5oc5ccccc56)cc4)c4ccccc4-c4ccccc4)c4oc5ccccc5c43)o2)cc1 ; c1ccc(-c2nc3c(o2)c(N(c2ccccc2)c2ccc(-c4cccc5c4sc4ccccc45)cc2)cc2oc4ccccc4c23)cc1]

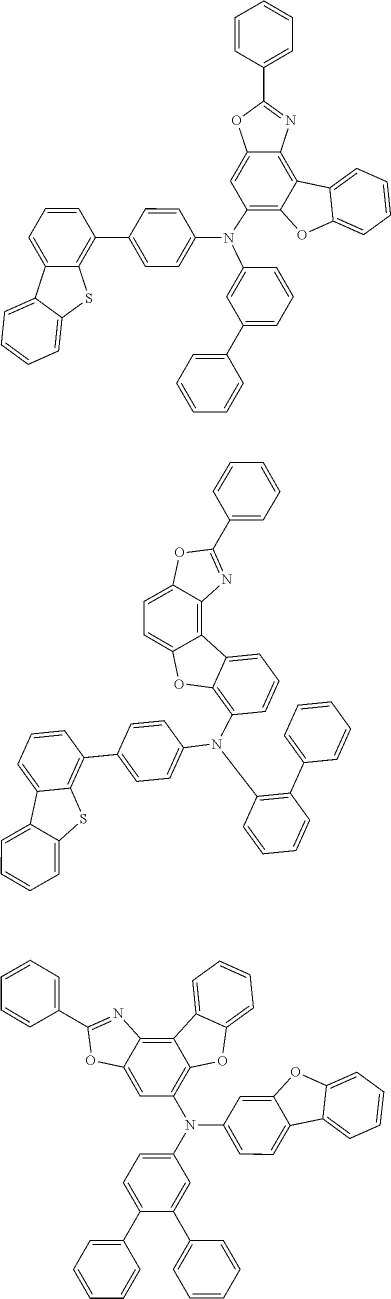 [SMILES: c1ccc(-c2cccc(N(c3ccc(-c4cccc5c4sc4ccccc45)cc3)c3cc4oc(-c5ccccc5)nc4c4c3oc3ccccc34)c2)cc1 ; c1ccc(-c2nc3c(ccc4oc5c(N(c6ccc(-c7cccc8c7sc7ccccc78)cc6)c6ccccc6-c6ccccc6)cccc5c43)o2)cc1 ; c1ccc(-c2nc3c(cc(N(c4ccc(-c5ccccc5)c(-c5ccccc5)c4)c4ccc5c(c4)oc4ccccc45)c4oc5ccccc5c43)o2)cc1]

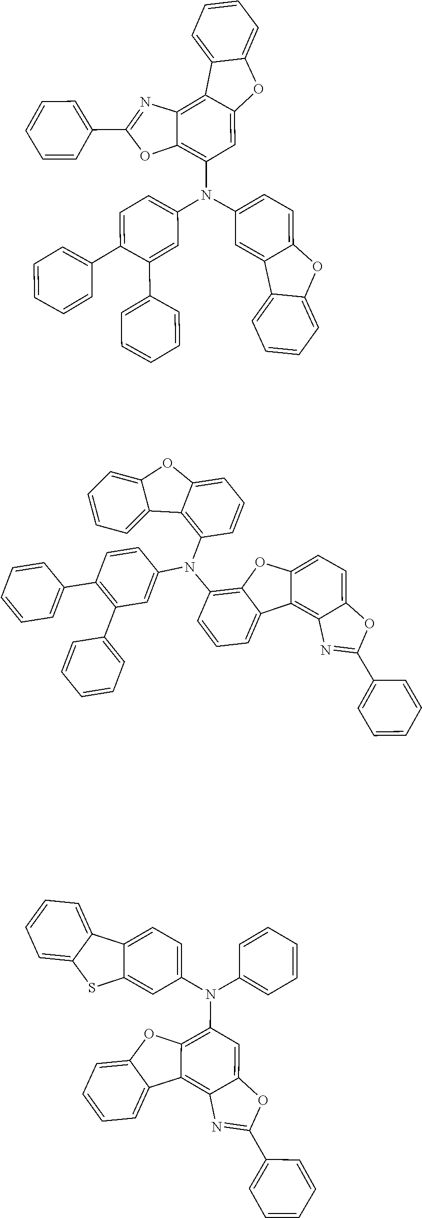 [SMILES: c1ccc(-c2nc3c(o2)c(N(c2ccc(-c4ccccc4)c(-c4ccccc4)c2)c2ccc4oc5ccccc5c4c2)cc2oc4ccccc4c23)cc1 ; c1ccc(-c2nc3c(ccc4oc5c(N(c6ccc(-c7ccccc7)c(-c7ccccc7)c6)c6cccc7oc8ccccc8c67)cccc5c43)o2)cc1 ; c1ccc(-c2nc3c(cc(N(c4ccccc4)c4ccc5c(c4)sc4ccccc45)c4oc5ccccc5c43)o2)cc1]

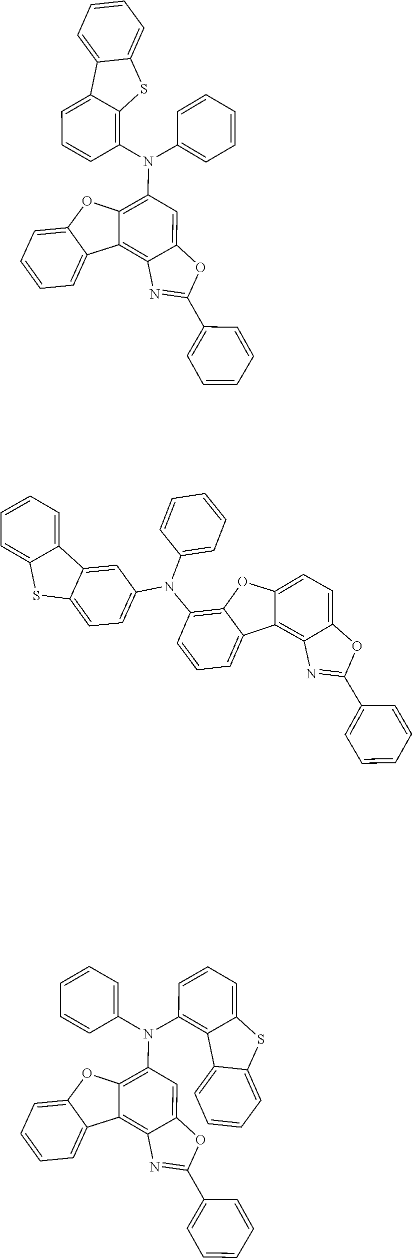 [SMILES: c1ccc(-c2nc3c(cc(N(c4ccccc4)c4cccc5c4sc4ccccc45)c4oc5ccccc5c43)o2)cc1 ; c1ccc(-c2nc3c(ccc4oc5c(N(c6ccccc6)c6ccc7sc8ccccc8c7c6)cccc5c43)o2)cc1 ; c1ccc(-c2nc3c(cc(N(c4ccccc4)c4cccc5sc6ccccc6c45)c4oc5ccccc5c43)o2)cc1]

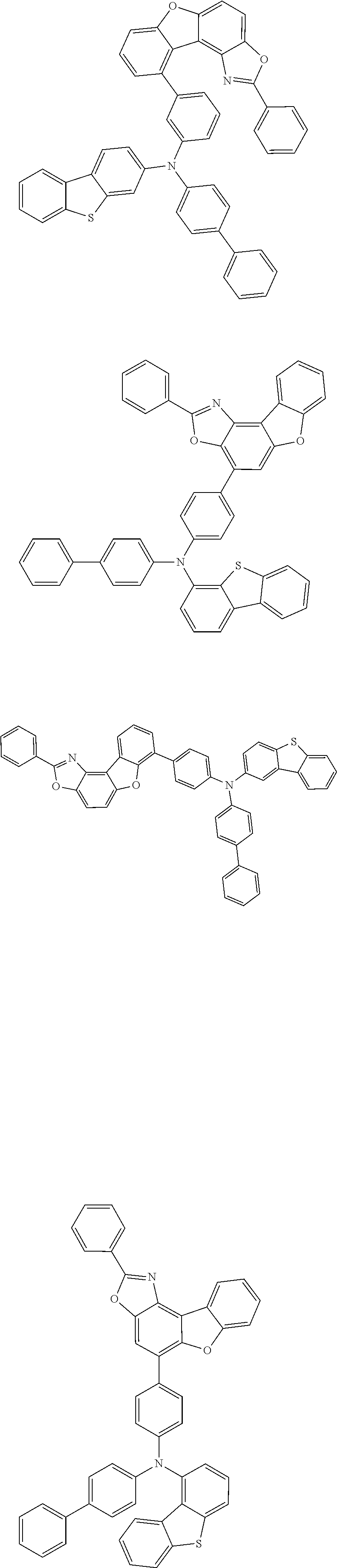 [SMILES: c1ccc(-c2ccc(N(c3cccc(-c4cccc5oc6ccc7oc(-c8ccccc8)nc7c6c45)c3)c3ccc4c(c3)sc3ccccc34)cc2)cc1 ; c1ccc(-c2ccc(N(c3ccc(-c4cc5oc6ccccc6c5c5nc(-c6ccccc6)oc45)cc3)c3cccc4c3sc3ccccc34)cc2)cc1 ; c1ccc(-c2ccc(N(c3ccc(-c4cccc5c4oc4ccc6oc(-c7ccccc7)nc6c45)cc3)c3ccc4sc5ccccc5c4c3)cc2)cc1 ; c1ccc(-c2ccc(N(c3ccc(-c4cc5oc(-c6ccccc6)nc5c5c4oc4ccccc45)cc3)c3cccc4sc5ccccc5c34)cc2)cc1]

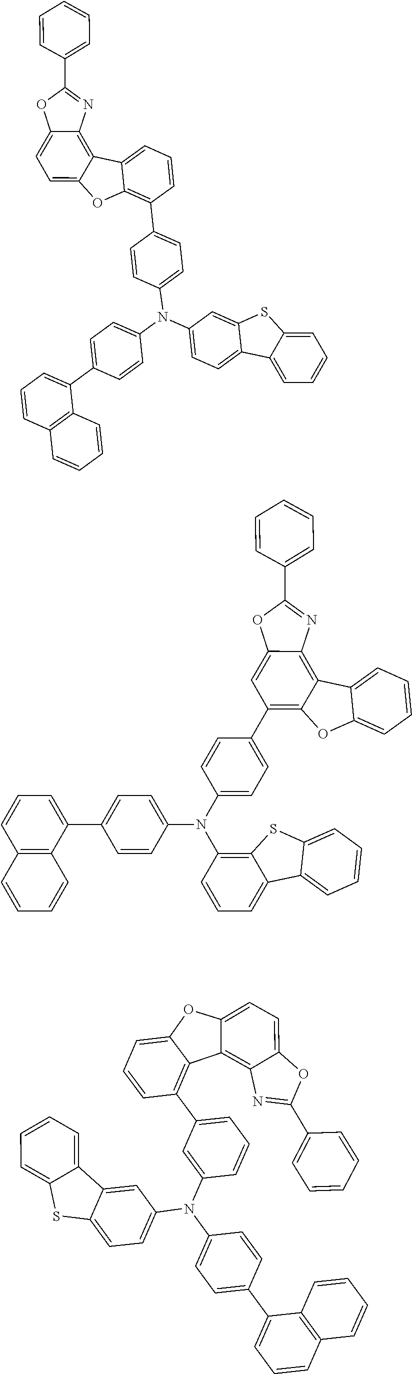 [SMILES: c1ccc(-c2nc3c(ccc4oc5c(-c6ccc(N(c7ccc(-c8cccc9ccccc89)cc7)c7ccc8c(c7)sc7ccccc78)cc6)cccc5c43)o2)cc1 ; c1ccc(-c2nc3c(cc(-c4ccc(N(c5ccc(-c6cccc7ccccc67)cc5)c5cccc6c5sc5ccccc56)cc4)c4oc5ccccc5c43)o2)cc1 ; c1ccc(-c2nc3c(ccc4oc5cccc(-c6cccc(N(c7ccc(-c8cccc9ccccc89)cc7)c7ccc8sc9ccccc9c8c7)c6)c5c43)o2)cc1]

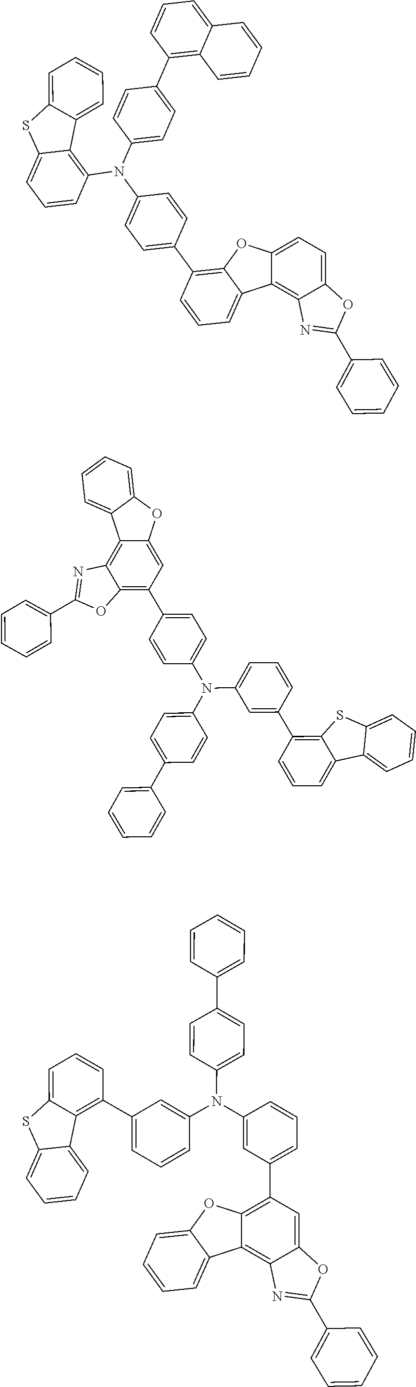 [SMILES: c1ccc(-c2nc3c(ccc4oc5c(-c6ccc(N(c7ccc(-c8cccc9ccccc89)cc7)c7cccc8sc9ccccc9c78)cc6)cccc5c43)o2)cc1 ; c1ccc(-c2ccc(N(c3ccc(-c4cc5oc6ccccc6c5c5nc(-c6ccccc6)oc45)cc3)c3cccc(-c4cccc5c4sc4ccccc45)c3)cc2)cc1 ; c1ccc(-c2ccc(N(c3cccc(-c4cc5oc(-c6ccccc6)nc5c5c4oc4ccccc45)c3)c3cccc(-c4cccc5sc6ccccc6c45)c3)cc2)cc1]

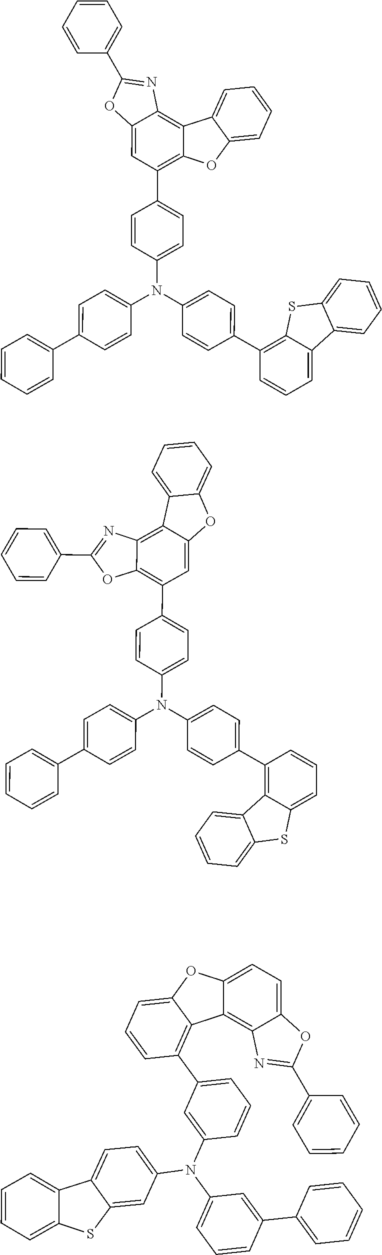 [SMILES: c1ccc(-c2ccc(N(c3ccc(-c4cc5oc(-c6ccccc6)nc5c5c4oc4ccccc45)cc3)c3ccc(-c4cccc5c4sc4ccccc45)cc3)cc2)cc1 ; c1ccc(-c2ccc(N(c3ccc(-c4cc5oc6ccccc6c5c5nc(-c6ccccc6)oc45)cc3)c3ccc(-c4cccc5sc6ccccc6c45)cc3)cc2)cc1 ; c1ccc(-c2cccc(N(c3cccc(-c4cccc5oc6ccc7oc(-c8ccccc8)nc7c6c45)c3)c3ccc4c(c3)sc3ccccc34)c2)cc1]

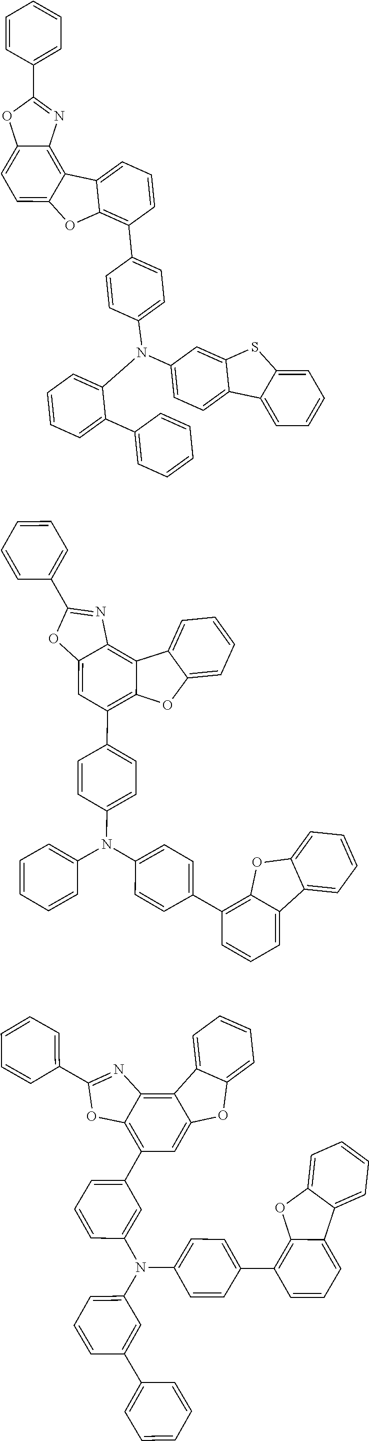 [SMILES: c1ccc(-c2nc3c(ccc4oc5c(-c6ccc(N(c7ccc8c(c7)sc7ccccc78)c7ccccc7-c7ccccc7)cc6)cccc5c43)o2)cc1 ; c1ccc(-c2nc3c(cc(-c4ccc(N(c5ccccc5)c5ccc(-c6cccc7c6oc6ccccc67)cc5)cc4)c4oc5ccccc5c43)o2)cc1 ; c1ccc(-c2cccc(N(c3ccc(-c4cccc5c4oc4ccccc45)cc3)c3cccc(-c4cc5oc6ccccc6c5c5nc(-c6ccccc6)oc45)c3)c2)cc1]

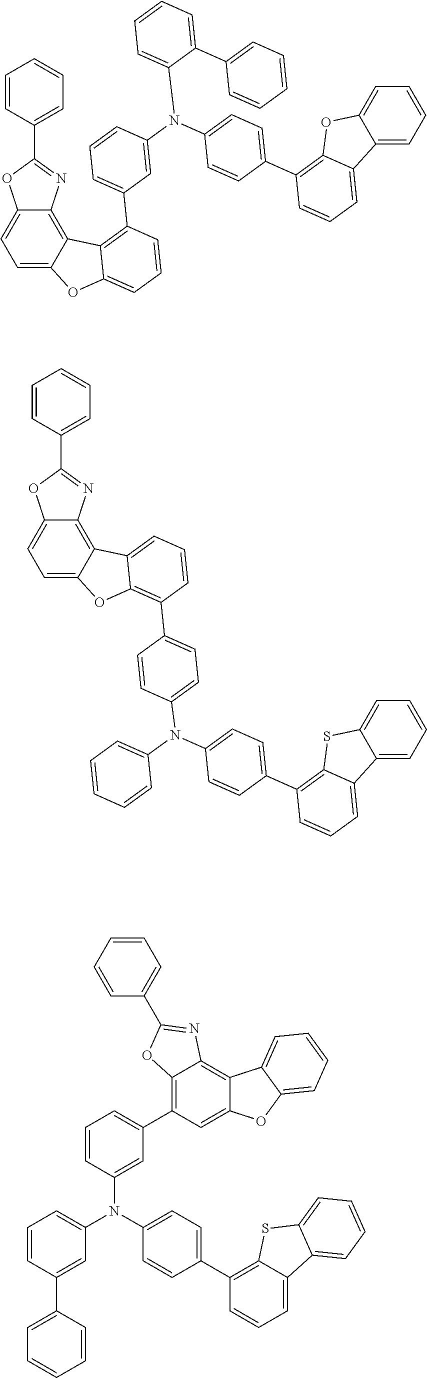 [SMILES: c1ccc(-c2nc3c(ccc4oc5cccc(-c6cccc(N(c7ccc(-c8cccc9c8oc8ccccc89)cc7)c7ccccc7-c7ccccc7)c6)c5c43)o2)cc1 ; c1ccc(-c2nc3c(ccc4oc5c(-c6ccc(N(c7ccccc7)c7ccc(-c8cccc9c8sc8ccccc89)cc7)cc6)cccc5c43)o2)cc1 ; c1ccc(-c2cccc(N(c3ccc(-c4cccc5c4sc4ccccc45)cc3)c3cccc(-c4cc5oc6ccccc6c5c5nc(-c6ccccc6)oc45)c3)c2)cc1]

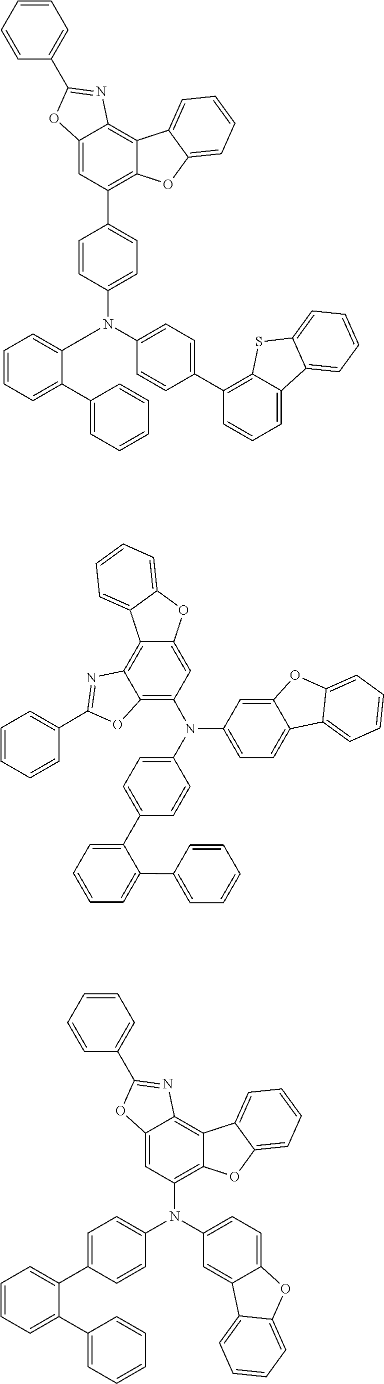 [SMILES: c1ccc(-c2nc3c(cc(-c4ccc(N(c5ccc(-c6cccc7c6sc6ccccc67)cc5)c5ccccc5-c5ccccc5)cc4)c4oc5ccccc5c43)o2)cc1 ; c1ccc(-c2nc3c(o2)c(N(c2ccc(-c4ccccc4-c4ccccc4)cc2)c2ccc4c(c2)oc2ccccc24)cc2oc4ccccc4c23)cc1 ; c1ccc(-c2nc3c(cc(N(c4ccc(-c5ccccc5-c5ccccc5)cc4)c4ccc5oc6ccccc6c5c4)c4oc5ccccc5c43)o2)cc1]

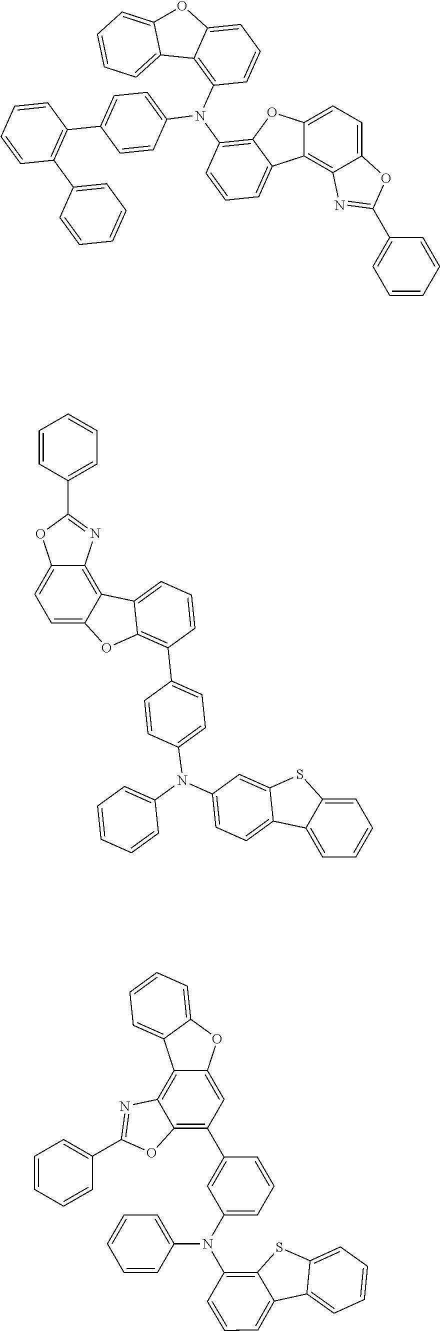 [SMILES: c1ccc(-c2nc3c(ccc4oc5c(N(c6ccc(-c7ccccc7-c7ccccc7)cc6)c6cccc7oc8ccccc8c67)cccc5c43)o2)cc1 ; c1ccc(-c2nc3c(ccc4oc5c(-c6ccc(N(c7ccccc7)c7ccc8c(c7)sc7ccccc78)cc6)cccc5c43)o2)cc1 ; c1ccc(-c2nc3c(o2)c(-c2cccc(N(c4ccccc4)c4cccc5c4sc4ccccc45)c2)cc2oc4ccccc4c23)cc1]

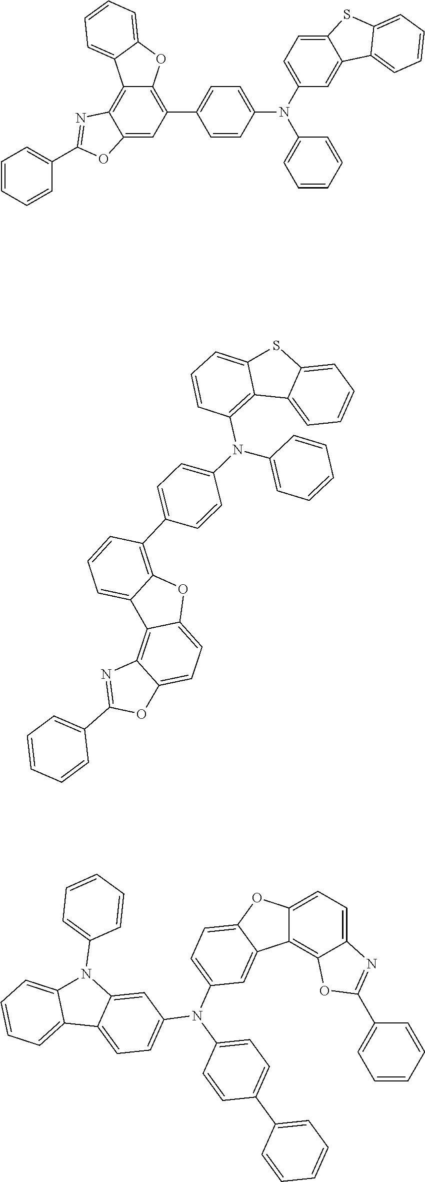 [SMILES: c1ccc(-c2nc3c(cc(-c4ccc(N(c5ccccc5)c5ccc6sc7ccccc7c6c5)cc4)c4oc5ccccc5c43)o2)cc1 ; c1ccc(-c2nc3c(ccc4oc5c(-c6ccc(N(c7ccccc7)c7cccc8sc9ccccc9c78)cc6)cccc5c43)o2)cc1 ; c1ccc(-c2ccc(N(c3ccc4oc5ccc6nc(-c7ccccc7)oc6c5c4c3)c3ccc4c5ccccc5n(-c5ccccc5)c4c3)cc2)cc1]

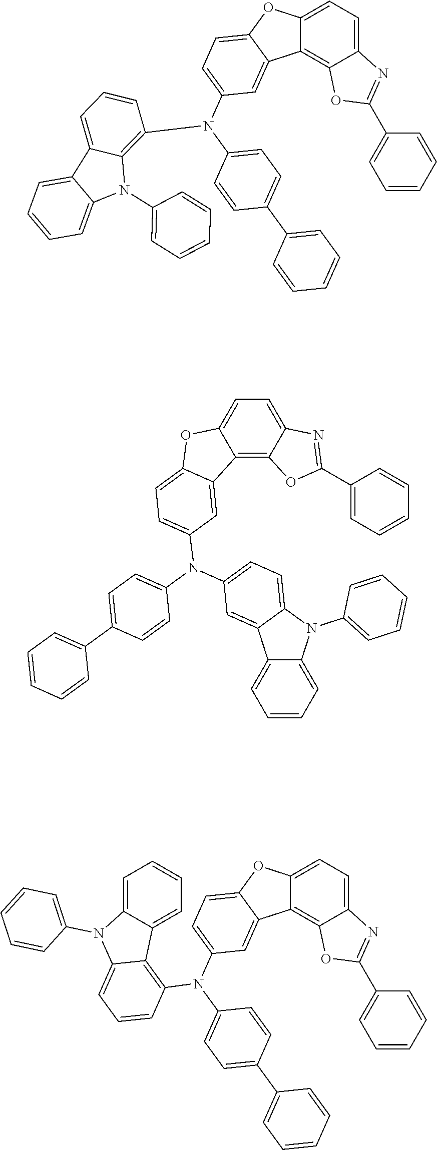 [SMILES: c1ccc(-c2ccc(N(c3ccc4oc5ccc6nc(-c7ccccc7)oc6c5c4c3)c3cccc4c5ccccc5n(-c5ccccc5)c34)cc2)cc1 ; c1ccc(-c2ccc(N(c3ccc4oc5ccc6nc(-c7ccccc7)oc6c5c4c3)c3ccc4c(c3)c3ccccc3n4-c3ccccc3)cc2)cc1 ; c1ccc(-c2ccc(N(c3ccc4oc5ccc6nc(-c7ccccc7)oc6c5c4c3)c3cccc4c3c3ccccc3n4-c3ccccc3)cc2)cc1]

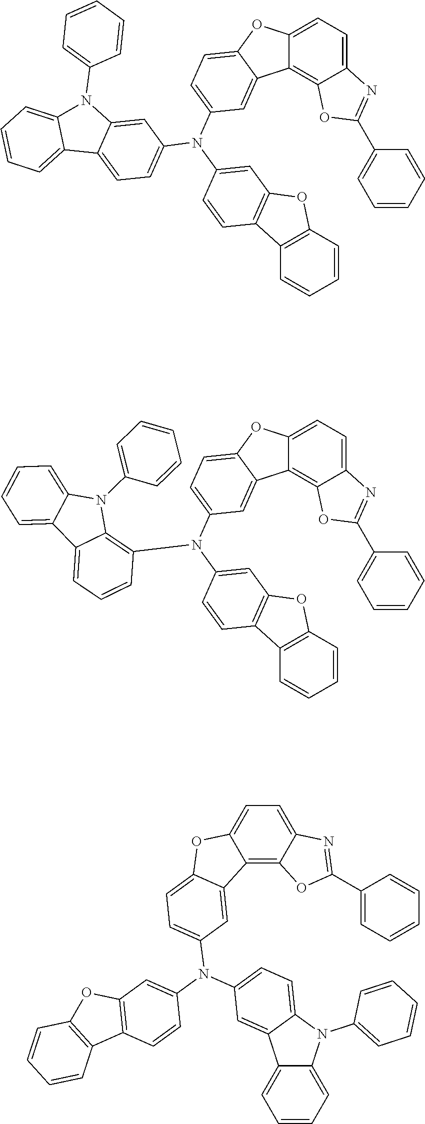 [SMILES: c1ccc(-c2nc3ccc4oc5ccc(N(c6ccc7c(c6)oc6ccccc67)c6ccc7c8ccccc8n(-c8ccccc8)c7c6)cc5c4c3o2)cc1 ; c1ccc(-c2nc3ccc4oc5ccc(N(c6ccc7c(c6)oc6ccccc67)c6cccc7c8ccccc8n(-c8ccccc8)c67)cc5c4c3o2)cc1 ; c1ccc(-c2nc3ccc4oc5ccc(N(c6ccc7c(c6)oc6ccccc67)c6ccc7c(c6)c6ccccc6n7-c6ccccc6)cc5c4c3o2)cc1]

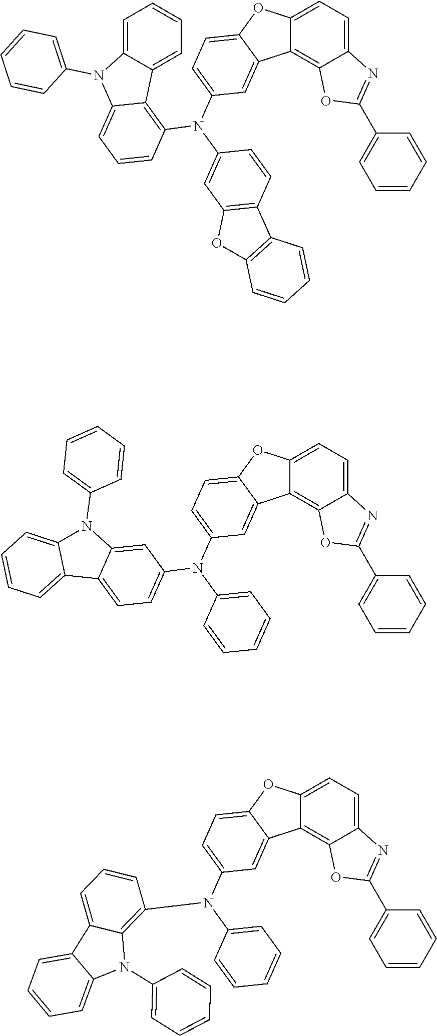 [SMILES: c1ccc(-c2nc3ccc4oc5ccc(N(c6ccc7c(c6)oc6ccccc67)c6cccc7c6c6ccccc6n7-c6ccccc6)cc5c4c3o2)cc1 ; c1ccc(-c2nc3ccc4oc5ccc(N(c6ccccc6)c6ccc7c8ccccc8n(-c8ccccc8)c7c6)cc5c4c3o2)cc1 ; c1ccc(-c2nc3ccc4oc5ccc(N(c6ccccc6)c6cccc7c8ccccc8n(-c8ccccc8)c67)cc5c4c3o2)cc1]

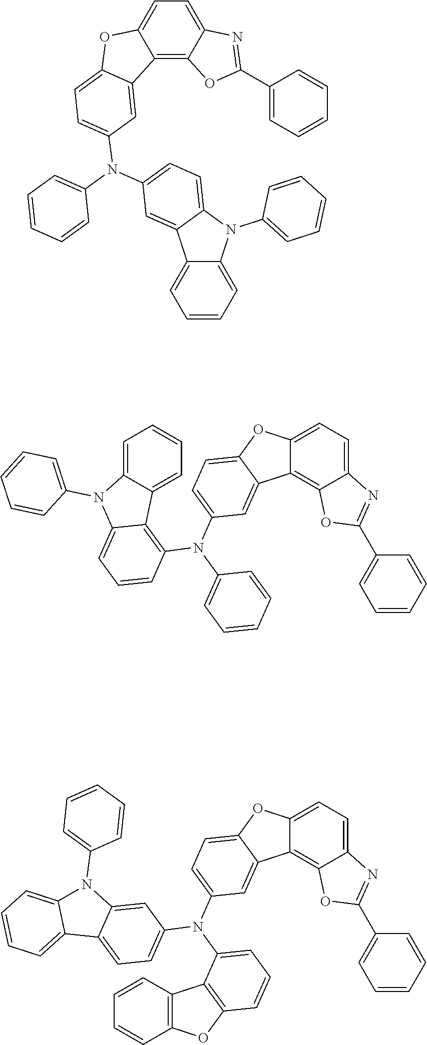 [SMILES: c1ccc(-c2nc3ccc4oc5ccc(N(c6ccccc6)c6ccc7c(c6)c6ccccc6n7-c6ccccc6)cc5c4c3o2)cc1 ; c1ccc(-c2nc3ccc4oc5ccc(N(c6ccccc6)c6cccc7c6c6ccccc6n7-c6ccccc6)cc5c4c3o2)cc1 ; c1ccc(-c2nc3ccc4oc5ccc(N(c6ccc7c8ccccc8n(-c8ccccc8)c7c6)c6cccc7oc8ccccc8c67)cc5c4c3o2)cc1]

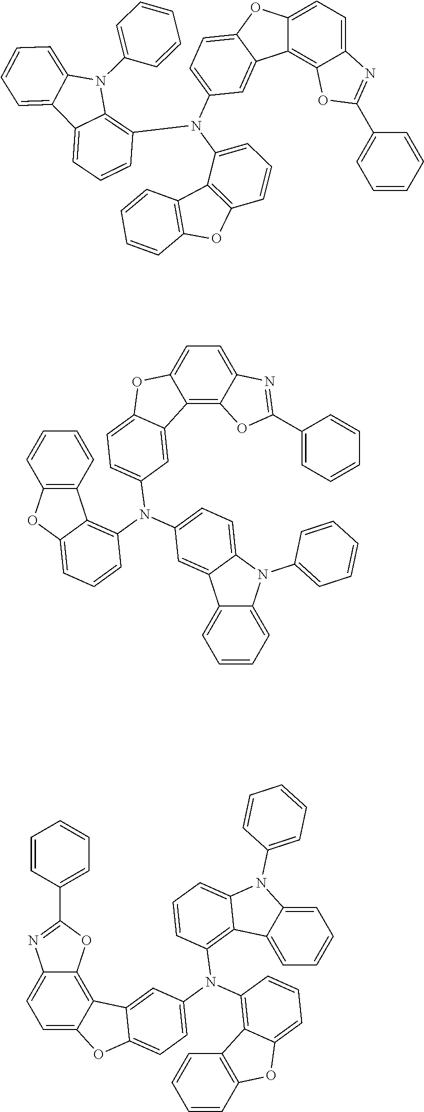 [SMILES: c1ccc(-c2nc3ccc4oc5ccc(N(c6cccc7oc8ccccc8c67)c6cccc7c8ccccc8n(-c8ccccc8)c67)cc5c4c3o2)cc1 ; c1ccc(-c2nc3ccc4oc5ccc(N(c6ccc7c(c6)c6ccccc6n7-c6ccccc6)c6cccc7oc8ccccc8c67)cc5c4c3o2)cc1 ; c1ccc(-c2nc3ccc4oc5ccc(N(c6cccc7oc8ccccc8c67)c6cccc7c6c6ccccc6n7-c6ccccc6)cc5c4c3o2)cc1]

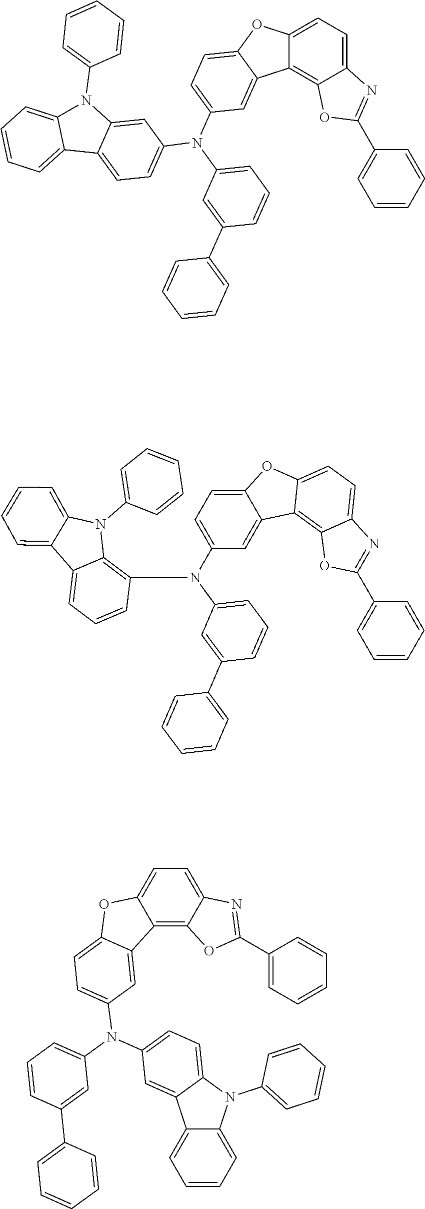 [SMILES: c1ccc(-c2cccc(N(c3ccc4oc5ccc6nc(-c7ccccc7)oc6c5c4c3)c3ccc4c5ccccc5n(-c5ccccc5)c4c3)c2)cc1 ; c1ccc(-c2cccc(N(c3ccc4oc5ccc6nc(-c7ccccc7)oc6c5c4c3)c3cccc4c5ccccc5n(-c5ccccc5)c34)c2)cc1 ; c1ccc(-c2cccc(N(c3ccc4oc5ccc6nc(-c7ccccc7)oc6c5c4c3)c3ccc4c(c3)c3ccccc3n4-c3ccccc3)c2)cc1]

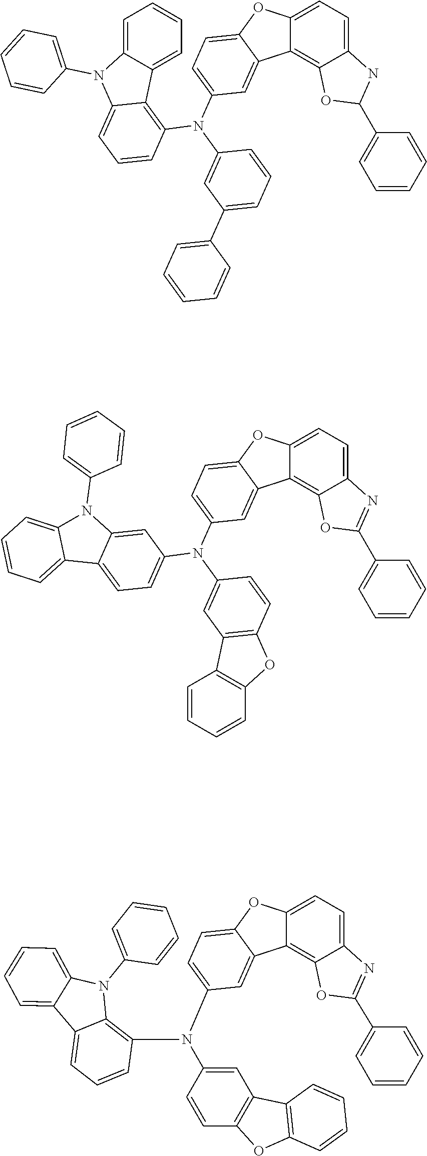

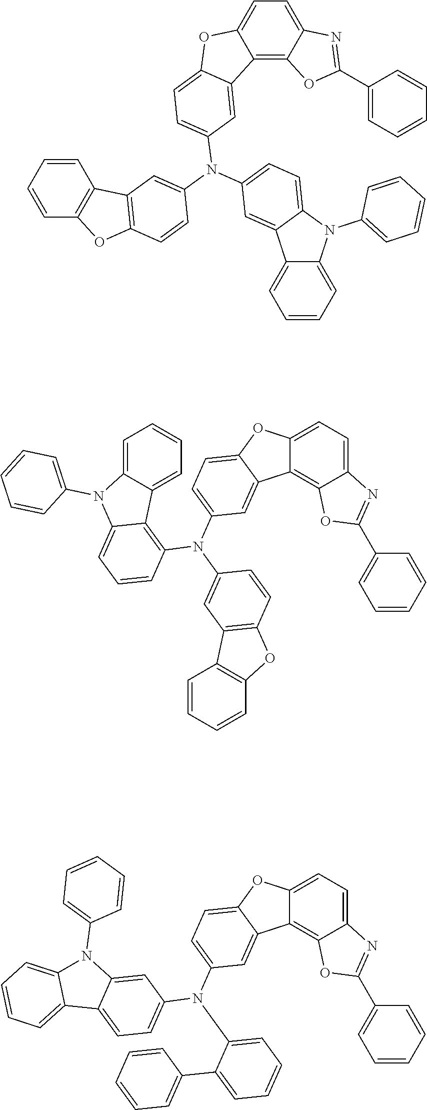 [SMILES: c1ccc(-c2nc3ccc4oc5ccc(N(c6ccc7oc8ccccc8c7c6)c6ccc7c(c6)c6ccccc6n7-c6ccccc6)cc5c4c3o2)cc1 ; c1ccc(-c2nc3ccc4oc5ccc(N(c6ccc7oc8ccccc8c7c6)c6cccc7c6c6ccccc6n7-c6ccccc6)cc5c4c3o2)cc1 ; c1ccc(-c2nc3ccc4oc5ccc(N(c6ccc7c8ccccc8n(-c8ccccc8)c7c6)c6ccccc6-c6ccccc6)cc5c4c3o2)cc1]

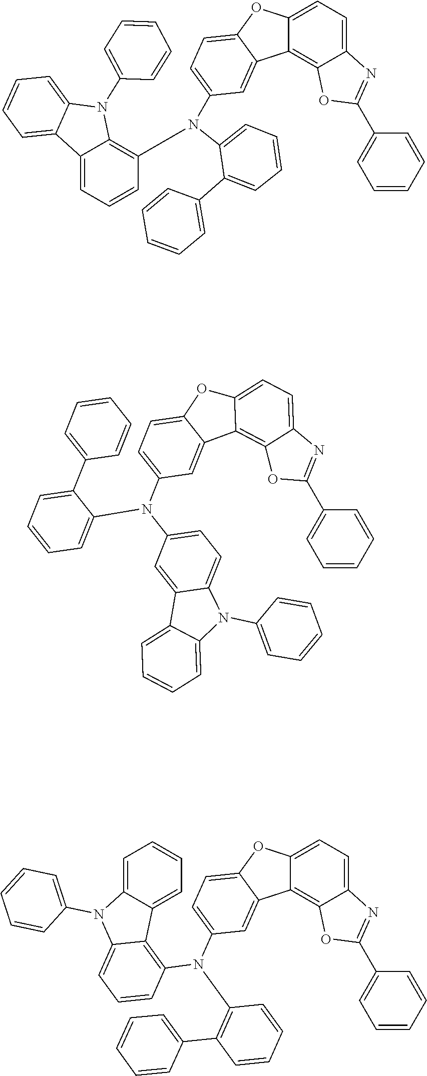 [SMILES: c1ccc(-c2nc3ccc4oc5ccc(N(c6ccccc6-c6ccccc6)c6cccc7c8ccccc8n(-c8ccccc8)c67)cc5c4c3o2)cc1 ; c1ccc(-c2nc3ccc4oc5ccc(N(c6ccc7c(c6)c6ccccc6n7-c6ccccc6)c6ccccc6-c6ccccc6)cc5c4c3o2)cc1 ; c1ccc(-c2nc3ccc4oc5ccc(N(c6ccccc6-c6ccccc6)c6cccc7c6c6ccccc6n7-c6ccccc6)cc5c4c3o2)cc1]

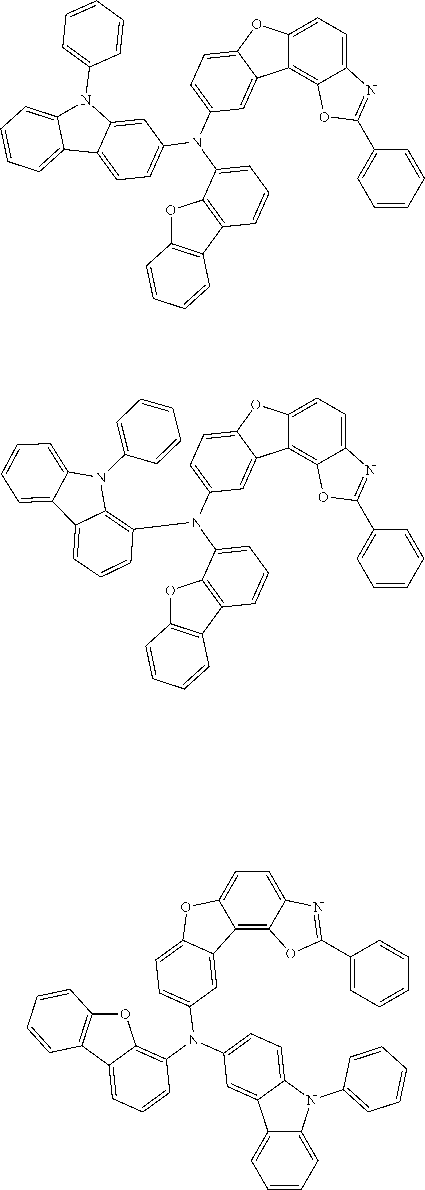 [SMILES: c1ccc(-c2nc3ccc4oc5ccc(N(c6ccc7c8ccccc8n(-c8ccccc8)c7c6)c6cccc7c6oc6ccccc67)cc5c4c3o2)cc1 ; c1ccc(-c2nc3ccc4oc5ccc(N(c6cccc7c6oc6ccccc67)c6cccc7c8ccccc8n(-c8ccccc8)c67)cc5c4c3o2)cc1 ; c1ccc(-c2nc3ccc4oc5ccc(N(c6ccc7c(c6)c6ccccc6n7-c6ccccc6)c6cccc7c6oc6ccccc67)cc5c4c3o2)cc1]

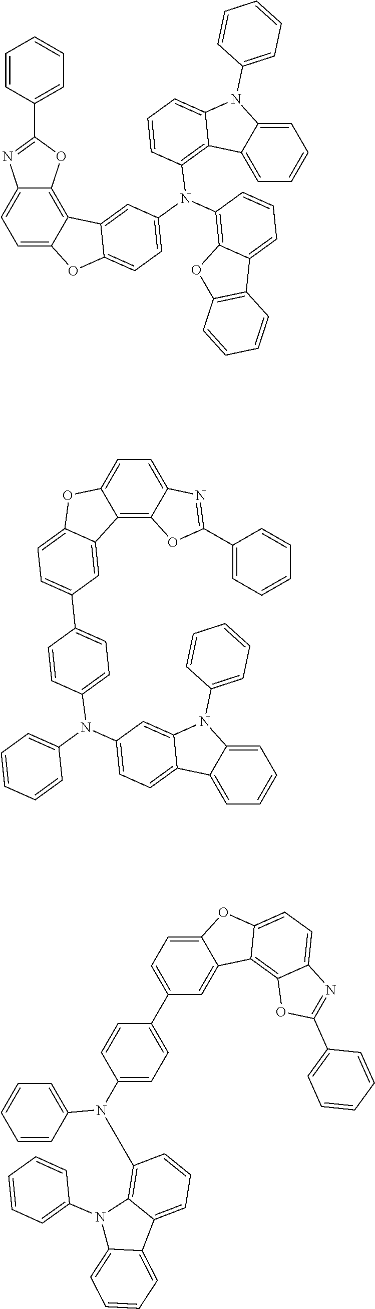 [SMILES: c1ccc(-c2nc3ccc4oc5ccc(N(c6cccc7c6oc6ccccc67)c6cccc7c6c6ccccc6n7-c6ccccc6)cc5c4c3o2)cc1 ; c1ccc(-c2nc3ccc4oc5ccc(-c6ccc(N(c7ccccc7)c7ccc8c9ccccc9n(-c9ccccc9)c8c7)cc6)cc5c4c3o2)cc1 ; c1ccc(-c2nc3ccc4oc5ccc(-c6ccc(N(c7ccccc7)c7cccc8c9ccccc9n(-c9ccccc9)c78)cc6)cc5c4c3o2)cc1]

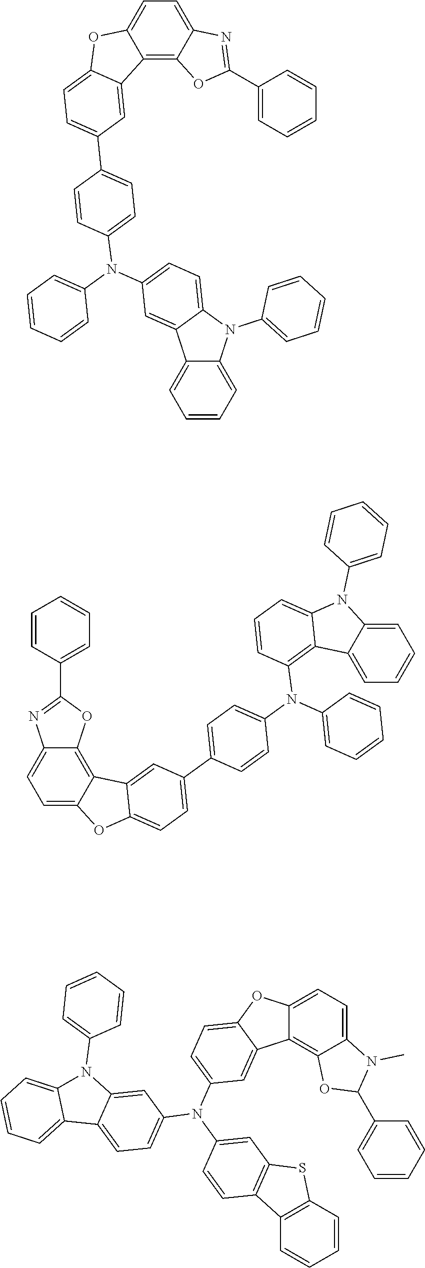 [SMILES: c1ccc(-c2nc3ccc4oc5ccc(-c6ccc(N(c7ccccc7)c7ccc8c(c7)c7ccccc7n8-c7ccccc7)cc6)cc5c4c3o2)cc1 ; c1ccc(-c2nc3ccc4oc5ccc(-c6ccc(N(c7ccccc7)c7cccc8c7c7ccccc7n8-c7ccccc7)cc6)cc5c4c3o2)cc1 ; CN1c2ccc3oc4ccc(N(c5ccc6c(c5)sc5ccccc56)c5ccc6c7ccccc7n(-c7ccccc7)c6c5)cc4c3c2OC1c1ccccc1]

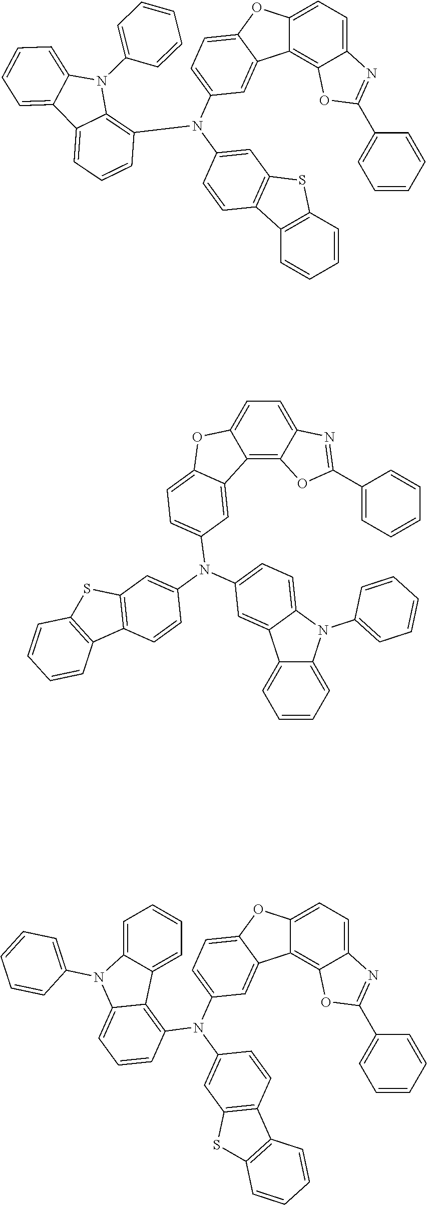 [SMILES: c1ccc(-c2nc3ccc4oc5ccc(N(c6ccc7c(c6)sc6ccccc67)c6cccc7c8ccccc8n(-c8ccccc8)c67)cc5c4c3o2)cc1 ; c1ccc(-c2nc3ccc4oc5ccc(N(c6ccc7c(c6)sc6ccccc67)c6ccc7c(c6)c6ccccc6n7-c6ccccc6)cc5c4c3o2)cc1 ; c1ccc(-c2nc3ccc4oc5ccc(N(c6ccc7c(c6)sc6ccccc67)c6cccc7c6c6ccccc6n7-c6ccccc6)cc5c4c3o2)cc1]

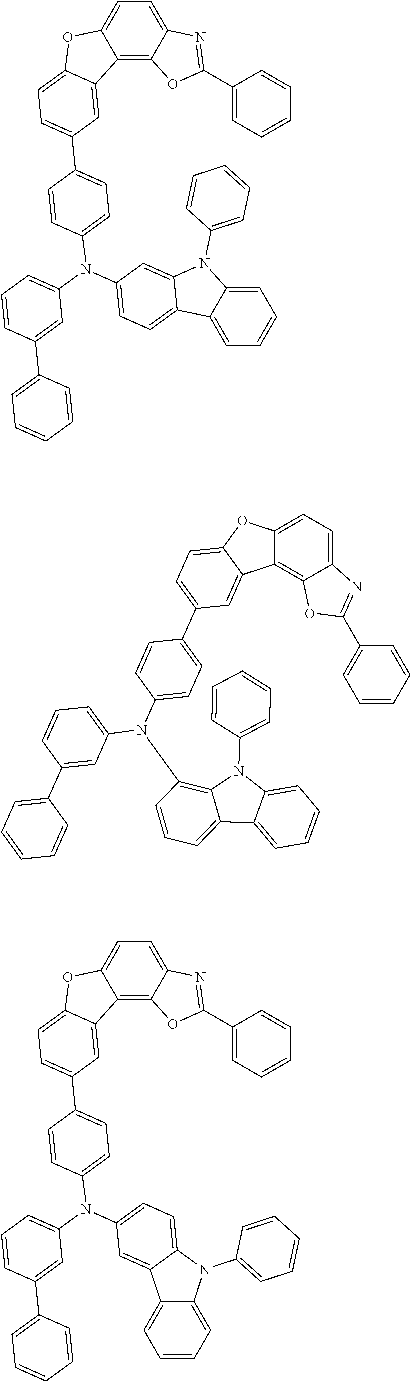 [SMILES: c1ccc(-c2cccc(N(c3ccc(-c4ccc5oc6ccc7nc(-c8ccccc8)oc7c6c5c4)cc3)c3ccc4c5ccccc5n(-c5ccccc5)c4c3)c2)cc1 ; c1ccc(-c2cccc(N(c3ccc(-c4ccc5oc6ccc7nc(-c8ccccc8)oc7c6c5c4)cc3)c3cccc4c5ccccc5n(-c5ccccc5)c34)c2)cc1 ; c1ccc(-c2cccc(N(c3ccc(-c4ccc5oc6ccc7nc(-c8ccccc8)oc7c6c5c4)cc3)c3ccc4c(c3)c3ccccc3n4-c3ccccc3)c2)cc1]

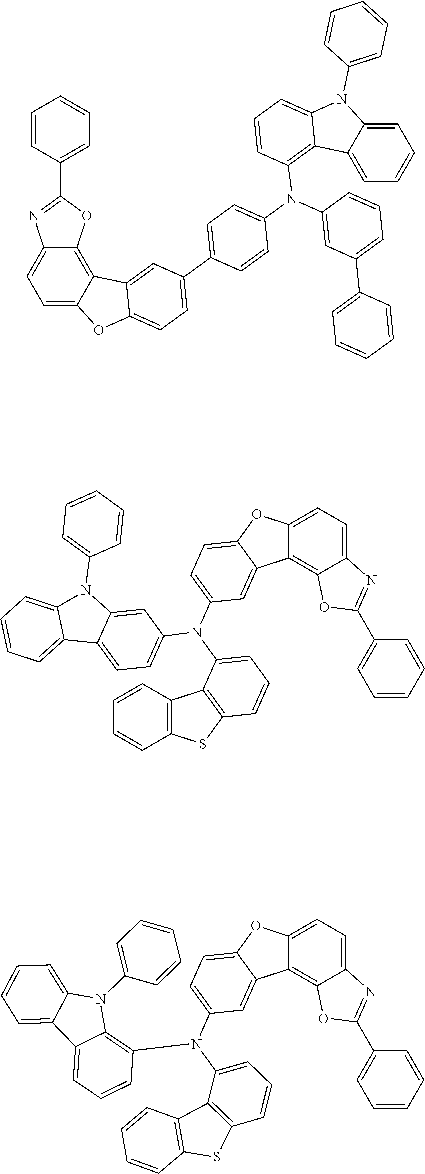 [SMILES: c1ccc(-c2cccc(N(c3ccc(-c4ccc5oc6ccc7nc(-c8ccccc8)oc7c6c5c4)cc3)c3cccc4c3c3ccccc3n4-c3ccccc3)c2)cc1 ; c1ccc(-c2nc3ccc4oc5ccc(N(c6ccc7c8ccccc8n(-c8ccccc8)c7c6)c6cccc7sc8ccccc8c67)cc5c4c3o2)cc1 ; c1ccc(-c2nc3ccc4oc5ccc(N(c6cccc7sc8ccccc8c67)c6cccc7c8ccccc8n(-c8ccccc8)c67)cc5c4c3o2)cc1]

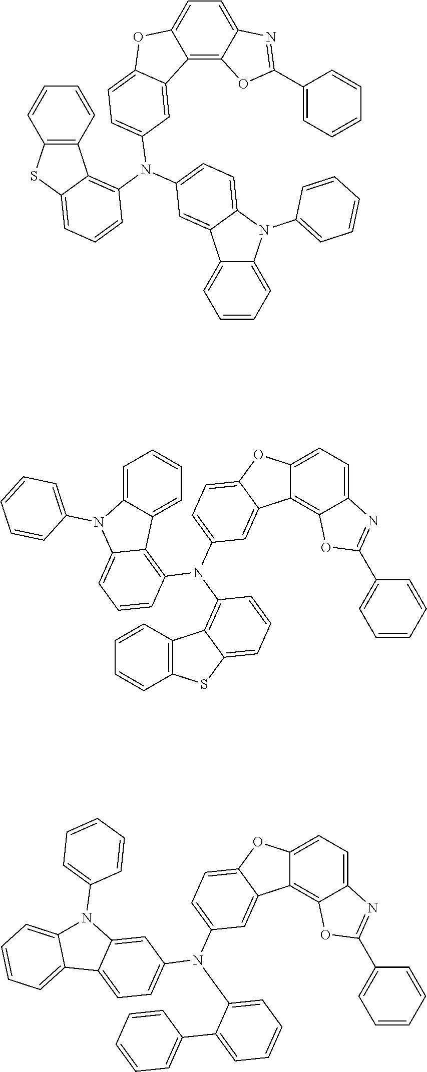 [SMILES: c1ccc(-c2nc3ccc4oc5ccc(N(c6ccc7c(c6)c6ccccc6n7-c6ccccc6)c6cccc7sc8ccccc8c67)cc5c4c3o2)cc1 ; c1ccc(-c2nc3ccc4oc5ccc(N(c6cccc7sc8ccccc8c67)c6cccc7c6c6ccccc6n7-c6ccccc6)cc5c4c3o2)cc1 ; c1ccc(-c2nc3ccc4oc5ccc(N(c6ccc7c8ccccc8n(-c8ccccc8)c7c6)c6ccccc6-c6ccccc6)cc5c4c3o2)cc1]

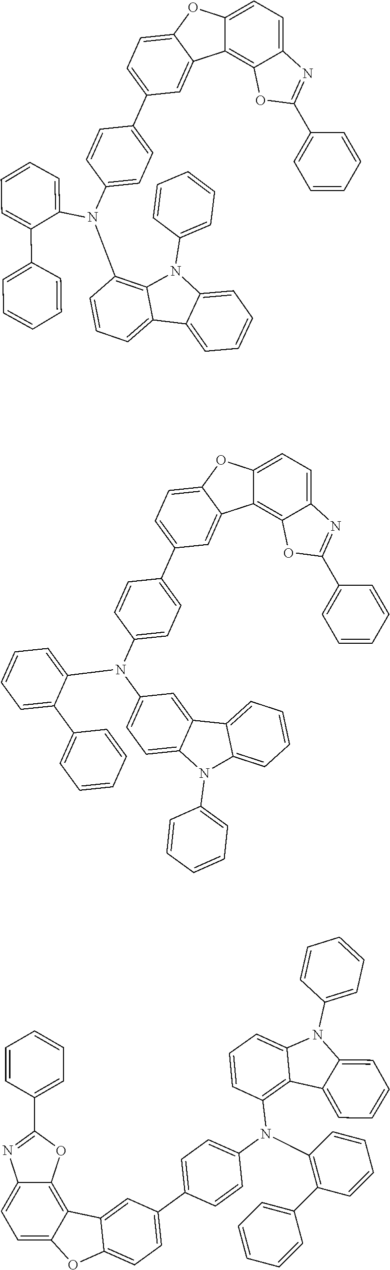 [SMILES: c1ccc(-c2nc3ccc4oc5ccc(-c6ccc(N(c7ccccc7-c7ccccc7)c7cccc8c9ccccc9n(-c9ccccc9)c78)cc6)cc5c4c3o2)cc1 ; c1ccc(-c2nc3ccc4oc5ccc(-c6ccc(N(c7ccc8c(c7)c7ccccc7n8-c7ccccc7)c7ccccc7-c7ccccc7)cc6)cc5c4c3o2)cc1 ; c1ccc(-c2nc3ccc4oc5ccc(-c6ccc(N(c7ccccc7-c7ccccc7)c7cccc8c7c7ccccc7n8-c7ccccc7)cc6)cc5c4c3o2)cc1]

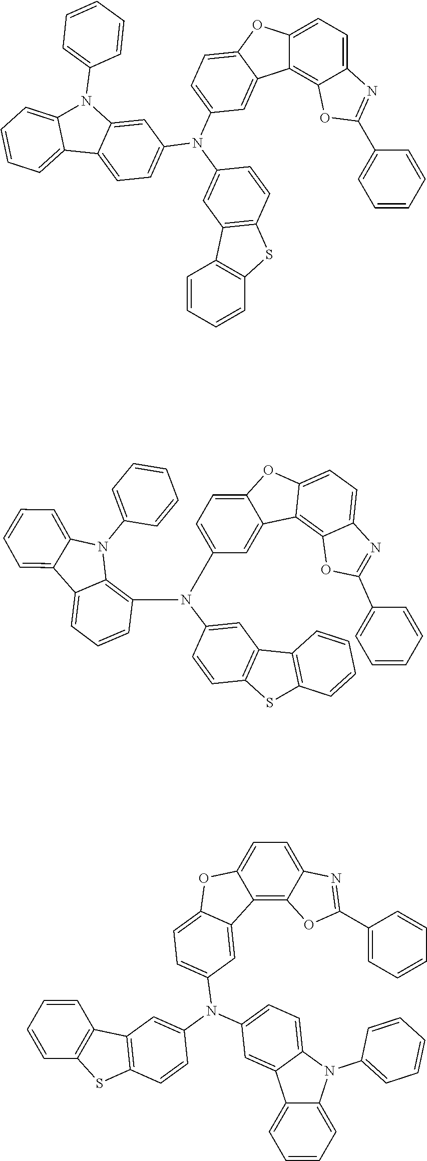 [SMILES: c1ccc(-c2nc3ccc4oc5ccc(N(c6ccc7sc8ccccc8c7c6)c6ccc7c8ccccc8n(-c8ccccc8)c7c6)cc5c4c3o2)cc1 ; c1ccc(-c2nc3ccc4oc5ccc(N(c6ccc7sc8ccccc8c7c6)c6cccc7c8ccccc8n(-c8ccccc8)c67)cc5c4c3o2)cc1 ; c1ccc(-c2nc3ccc4oc5ccc(N(c6ccc7sc8ccccc8c7c6)c6ccc7c(c6)c6ccccc6n7-c6ccccc6)cc5c4c3o2)cc1]

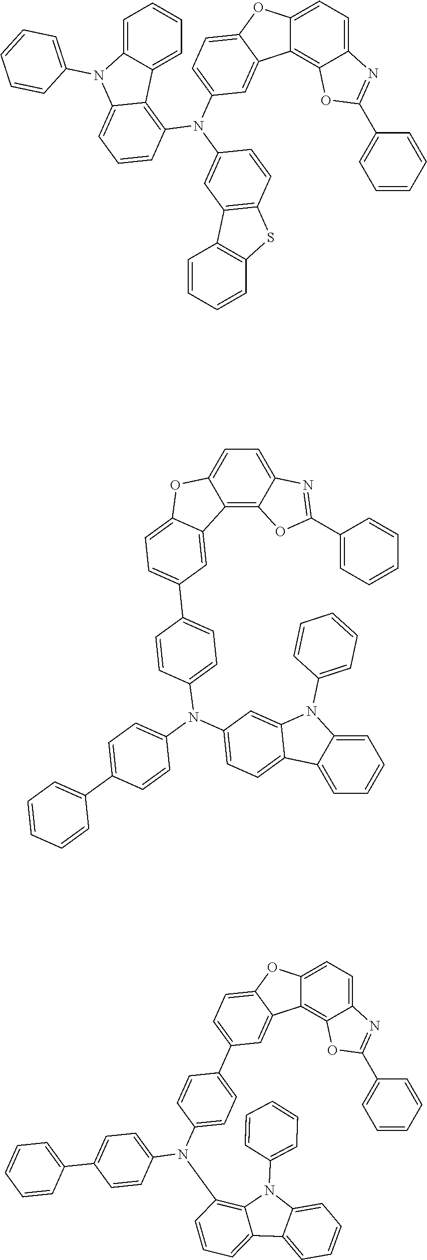 [SMILES: c1ccc(-c2nc3ccc4oc5ccc(N(c6ccc7sc8ccccc8c7c6)c6cccc7c6c6ccccc6n7-c6ccccc6)cc5c4c3o2)cc1 ; c1ccc(-c2ccc(N(c3ccc(-c4ccc5oc6ccc7nc(-c8ccccc8)oc7c6c5c4)cc3)c3ccc4c5ccccc5n(-c5ccccc5)c4c3)cc2)cc1 ; c1ccc(-c2ccc(N(c3ccc(-c4ccc5oc6ccc7nc(-c8ccccc8)oc7c6c5c4)cc3)c3cccc4c5ccccc5n(-c5ccccc5)c34)cc2)cc1]

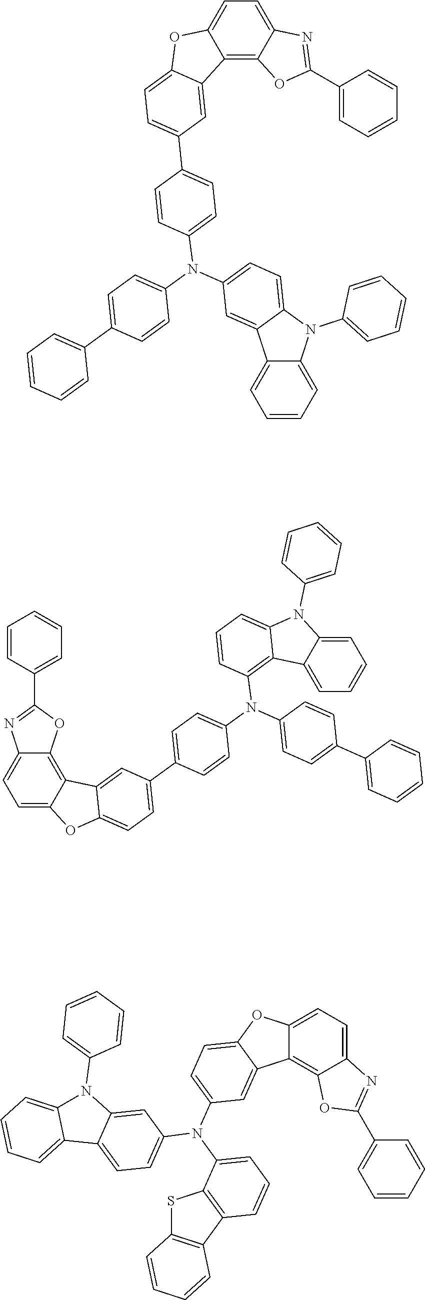 [SMILES: c1ccc(-c2ccc(N(c3ccc(-c4ccc5oc6ccc7nc(-c8ccccc8)oc7c6c5c4)cc3)c3ccc4c(c3)c3ccccc3n4-c3ccccc3)cc2)cc1 ; c1ccc(-c2ccc(N(c3ccc(-c4ccc5oc6ccc7nc(-c8ccccc8)oc7c6c5c4)cc3)c3cccc4c3c3ccccc3n4-c3ccccc3)cc2)cc1 ; c1ccc(-c2nc3ccc4oc5ccc(N(c6ccc7c8ccccc8n(-c8ccccc8)c7c6)c6cccc7c6sc6ccccc67)cc5c4c3o2)cc1]

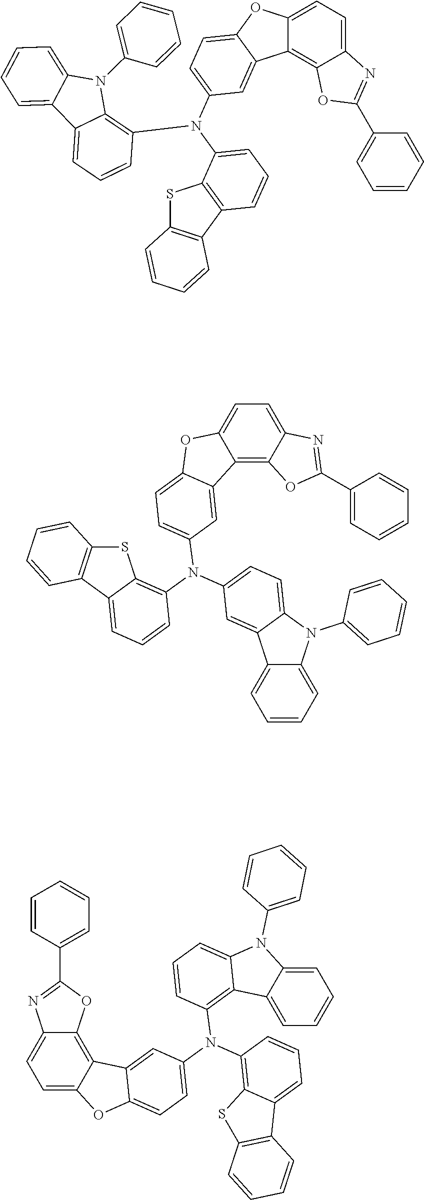 [SMILES: c1ccc(-c2nc3ccc4oc5ccc(N(c6cccc7c6sc6ccccc67)c6cccc7c8ccccc8n(-c8ccccc8)c67)cc5c4c3o2)cc1 ; c1ccc(-c2nc3ccc4oc5ccc(N(c6ccc7c(c6)c6ccccc6n7-c6ccccc6)c6cccc7c6sc6ccccc67)cc5c4c3o2)cc1 ; c1ccc(-c2nc3ccc4oc5ccc(N(c6cccc7c6sc6ccccc67)c6cccc7c6c6ccccc6n7-c6ccccc6)cc5c4c3o2)cc1]

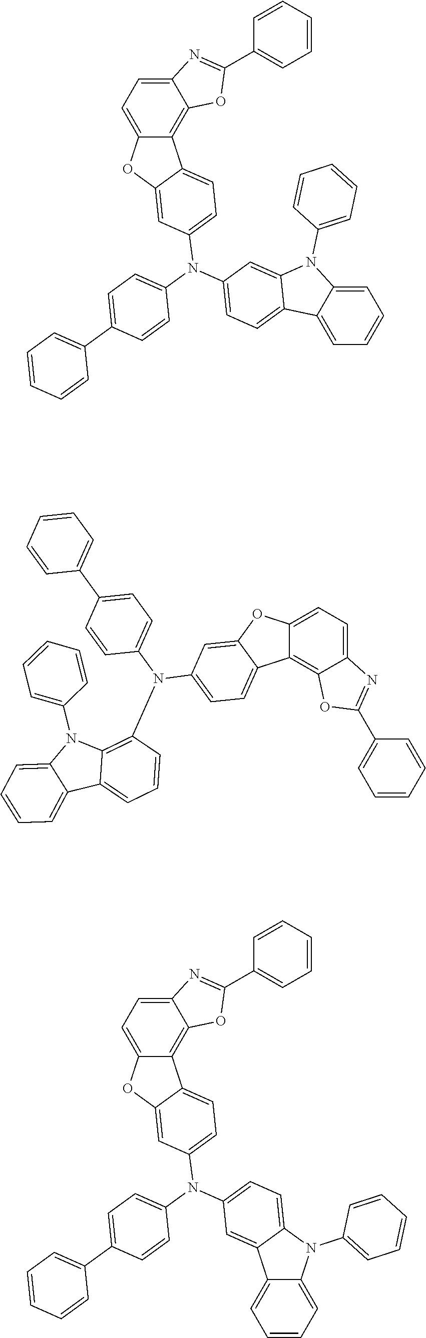 [SMILES: c1ccc(-c2ccc(N(c3ccc4c(c3)oc3ccc5nc(-c6ccccc6)oc5c34)c3ccc4c5ccccc5n(-c5ccccc5)c4c3)cc2)cc1 ; c1ccc(-c2ccc(N(c3ccc4c(c3)oc3ccc5nc(-c6ccccc6)oc5c34)c3cccc4c5ccccc5n(-c5ccccc5)c34)cc2)cc1 ; c1ccc(-c2ccc(N(c3ccc4c(c3)oc3ccc5nc(-c6ccccc6)oc5c34)c3ccc4c(c3)c3ccccc3n4-c3ccccc3)cc2)cc1]

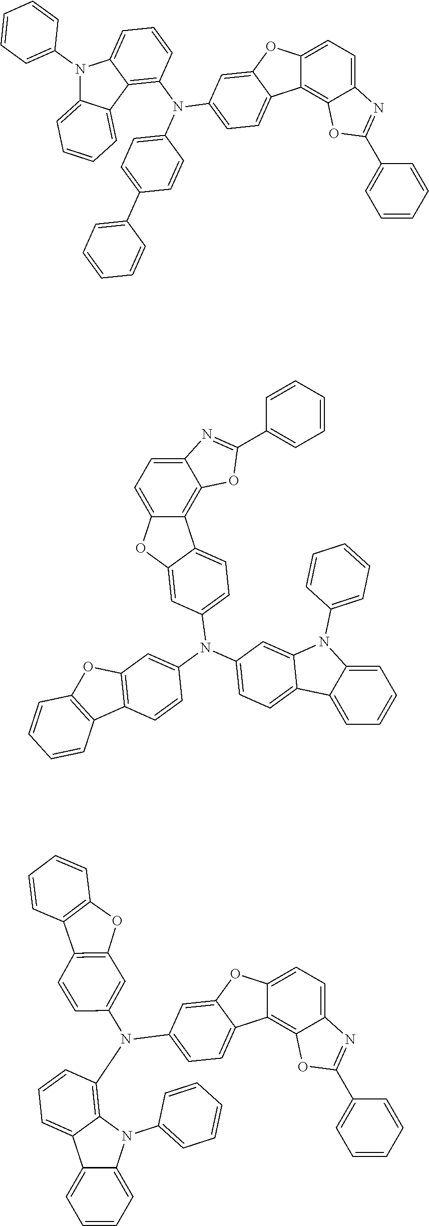 [SMILES: c1ccc(-c2ccc(N(c3ccc4c(c3)oc3ccc5nc(-c6ccccc6)oc5c34)c3cccc4c3c3ccccc3n4-c3ccccc3)cc2)cc1 ; c1ccc(-c2nc3ccc4oc5cc(N(c6ccc7c(c6)oc6ccccc67)c6ccc7c8ccccc8n(-c8ccccc8)c7c6)ccc5c4c3o2)cc1 ; c1ccc(-c2nc3ccc4oc5cc(N(c6ccc7c(c6)oc6ccccc67)c6cccc7c8ccccc8n(-c8ccccc8)c67)ccc5c4c3o2)cc1]

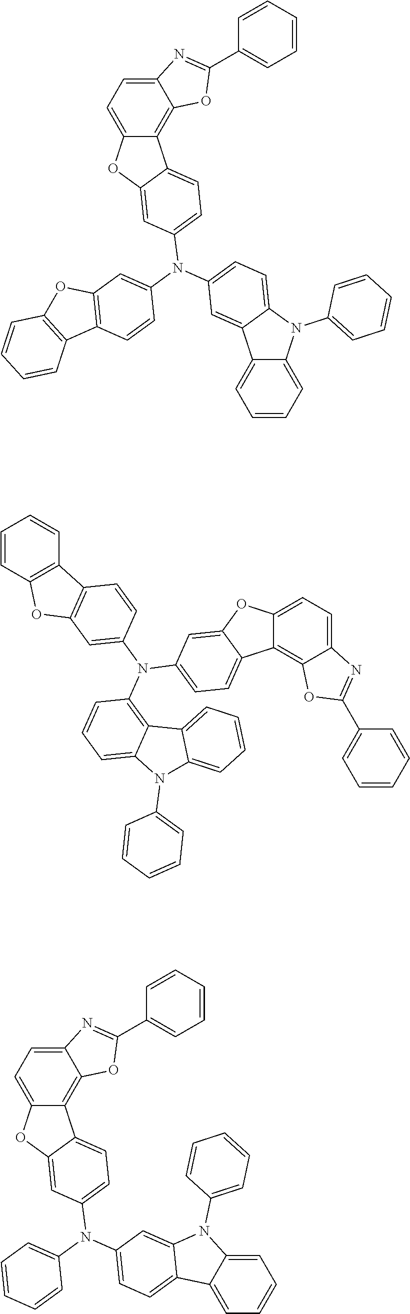 [SMILES: c1ccc(-c2nc3ccc4oc5cc(N(c6ccc7c(c6)oc6ccccc67)c6ccc7c(c6)c6ccccc6n7-c6ccccc6)ccc5c4c3o2)cc1 ; c1ccc(-c2nc3ccc4oc5cc(N(c6ccc7c(c6)oc6ccccc67)c6cccc7c6c6ccccc6n7-c6ccccc6)ccc5c4c3o2)cc1 ; c1ccc(-c2nc3ccc4oc5cc(N(c6ccccc6)c6ccc7c8ccccc8n(-c8ccccc8)c7c6)ccc5c4c3o2)cc1]

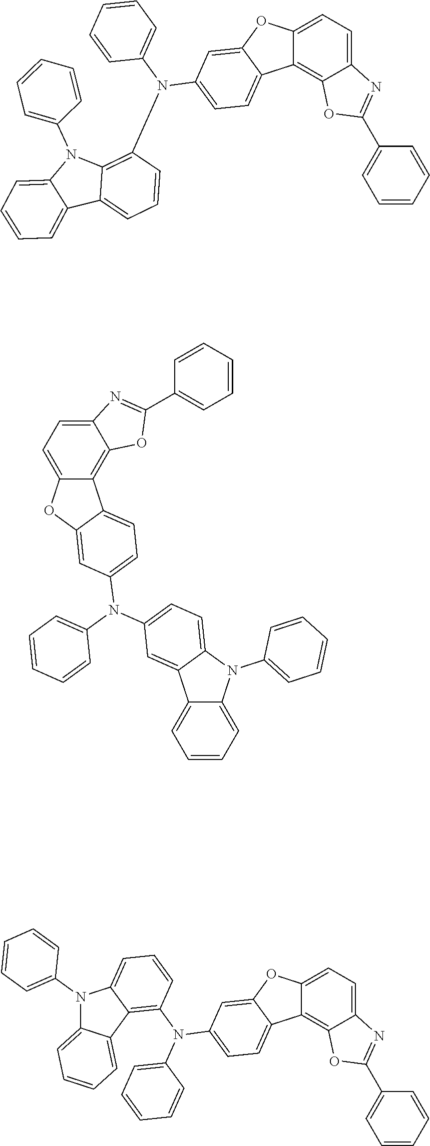 [SMILES: c1ccc(-c2nc3ccc4oc5cc(N(c6ccccc6)c6cccc7c8ccccc8n(-c8ccccc8)c67)ccc5c4c3o2)cc1 ; c1ccc(-c2nc3ccc4oc5cc(N(c6ccccc6)c6ccc7c(c6)c6ccccc6n7-c6ccccc6)ccc5c4c3o2)cc1 ; c1ccc(-c2nc3ccc4oc5cc(N(c6ccccc6)c6cccc7c6c6ccccc6n7-c6ccccc6)ccc5c4c3o2)cc1]

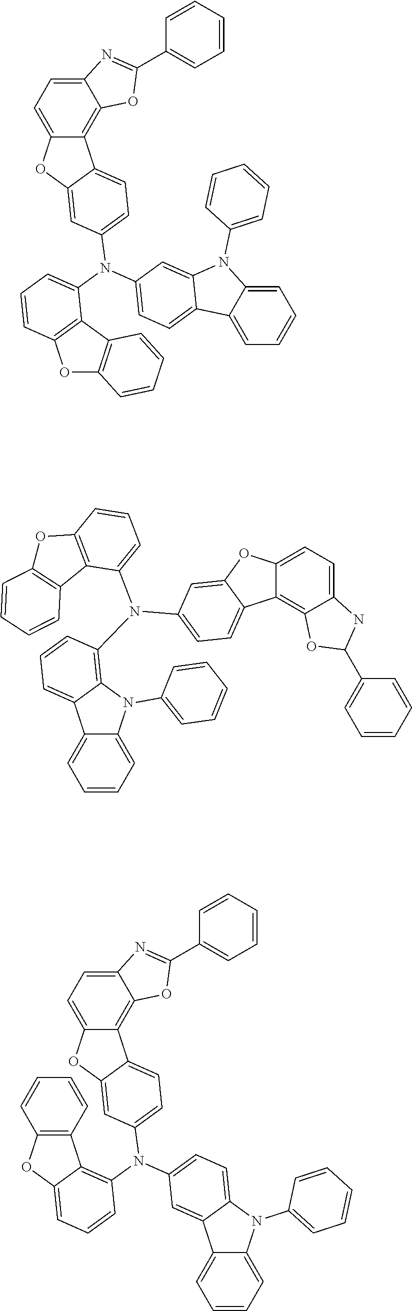

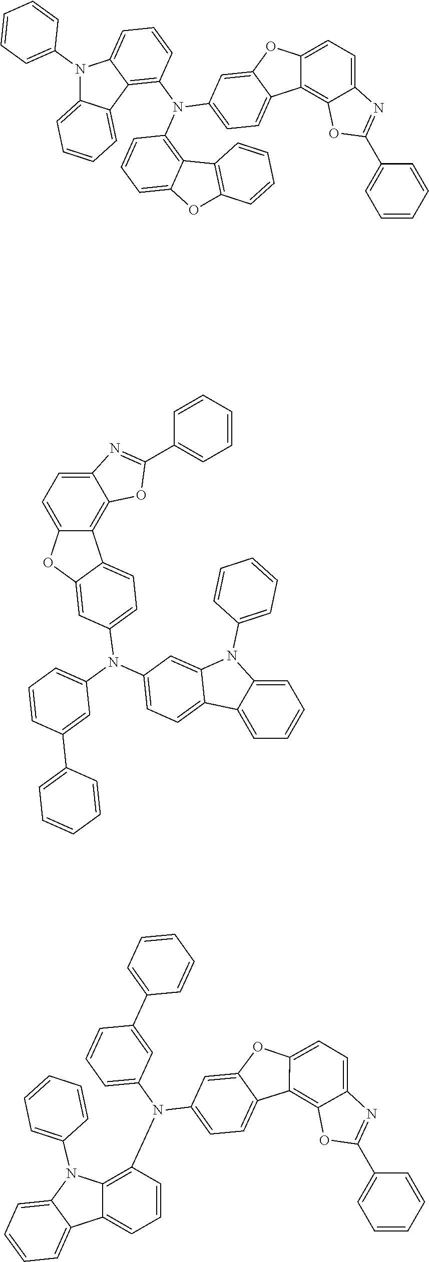 [SMILES: c1ccc(-c2nc3ccc4oc5cc(N(c6cccc7oc8ccccc8c67)c6cccc7c6c6ccccc6n7-c6ccccc6)ccc5c4c3o2)cc1 ; c1ccc(-c2cccc(N(c3ccc4c(c3)oc3ccc5nc(-c6ccccc6)oc5c34)c3ccc4c5ccccc5n(-c5ccccc5)c4c3)c2)cc1 ; c1ccc(-c2cccc(N(c3ccc4c(c3)oc3ccc5nc(-c6ccccc6)oc5c34)c3cccc4c5ccccc5n(-c5ccccc5)c34)c2)cc1]

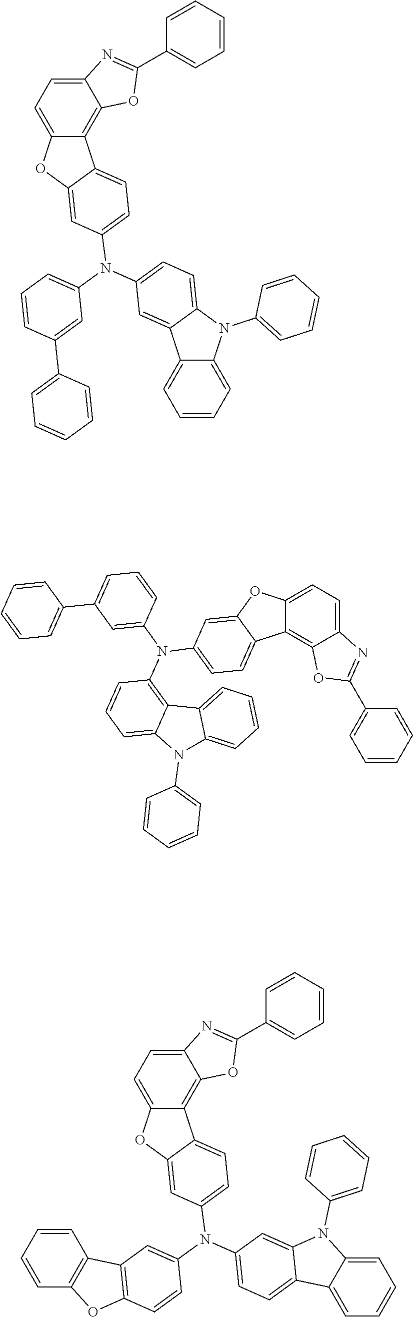 [SMILES: c1ccc(-c2cccc(N(c3ccc4c(c3)oc3ccc5nc(-c6ccccc6)oc5c34)c3ccc4c(c3)c3ccccc3n4-c3ccccc3)c2)cc1 ; c1ccc(-c2cccc(N(c3ccc4c(c3)oc3ccc5nc(-c6ccccc6)oc5c34)c3cccc4c3c3ccccc3n4-c3ccccc3)c2)cc1 ; c1ccc(-c2nc3ccc4oc5cc(N(c6ccc7oc8ccccc8c7c6)c6ccc7c8ccccc8n(-c8ccccc8)c7c6)ccc5c4c3o2)cc1]

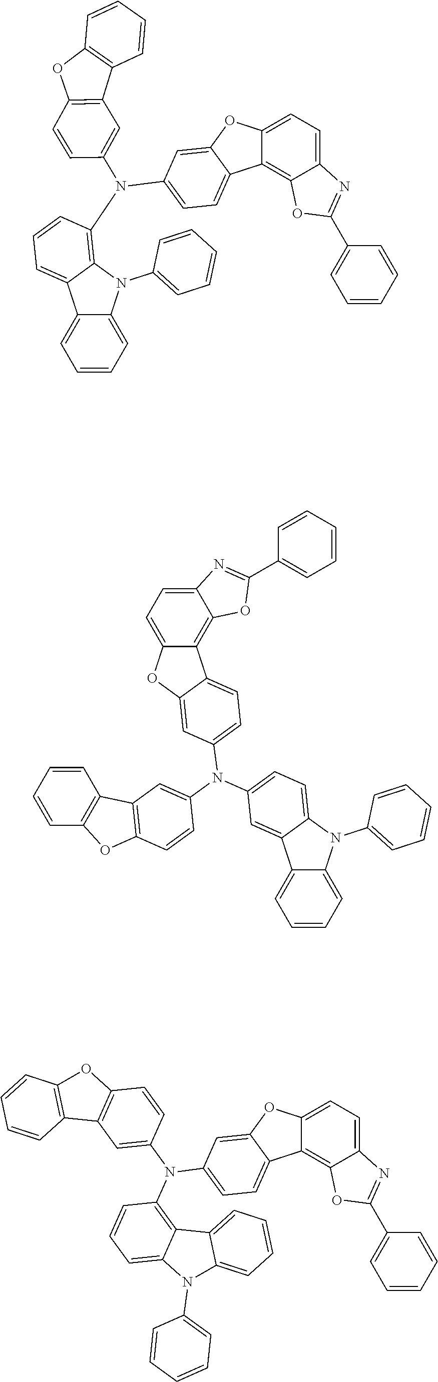 [SMILES: c1ccc(-c2nc3ccc4oc5cc(N(c6ccc7oc8ccccc8c7c6)c6cccc7c8ccccc8n(-c8ccccc8)c67)ccc5c4c3o2)cc1 ; c1ccc(-c2nc3ccc4oc5cc(N(c6ccc7oc8ccccc8c7c6)c6ccc7c(c6)c6ccccc6n7-c6ccccc6)ccc5c4c3o2)cc1 ; c1ccc(-c2nc3ccc4oc5cc(N(c6ccc7oc8ccccc8c7c6)c6cccc7c6c6ccccc6n7-c6ccccc6)ccc5c4c3o2)cc1]

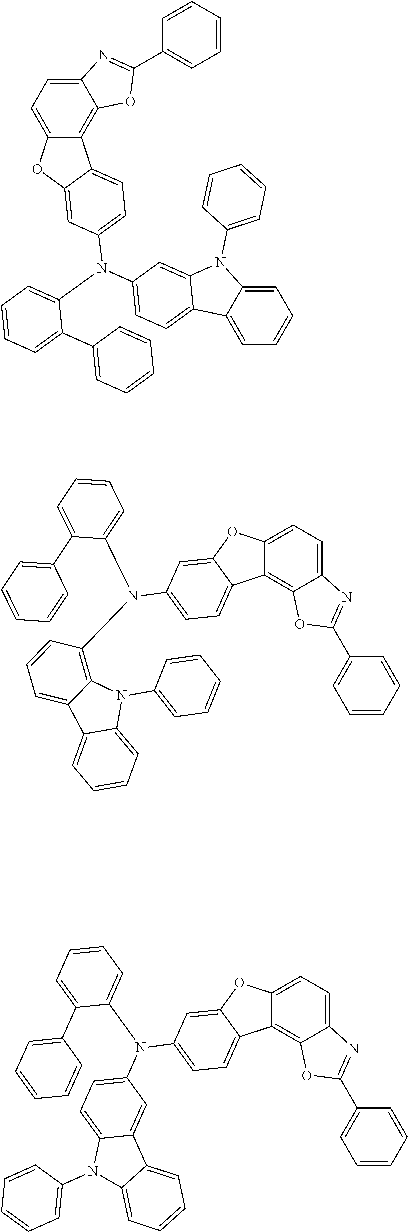 [SMILES: c1ccc(-c2nc3ccc4oc5cc(N(c6ccc7c8ccccc8n(-c8ccccc8)c7c6)c6ccccc6-c6ccccc6)ccc5c4c3o2)cc1 ; c1ccc(-c2nc3ccc4oc5cc(N(c6ccccc6-c6ccccc6)c6cccc7c8ccccc8n(-c8ccccc8)c67)ccc5c4c3o2)cc1 ; c1ccc(-c2nc3ccc4oc5cc(N(c6ccc7c(c6)c6ccccc6n7-c6ccccc6)c6ccccc6-c6ccccc6)ccc5c4c3o2)cc1]

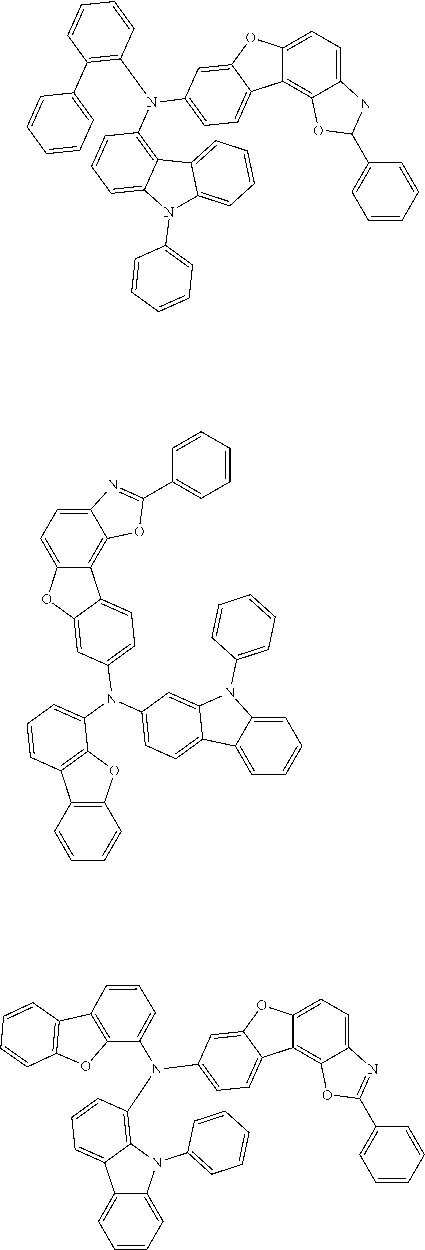

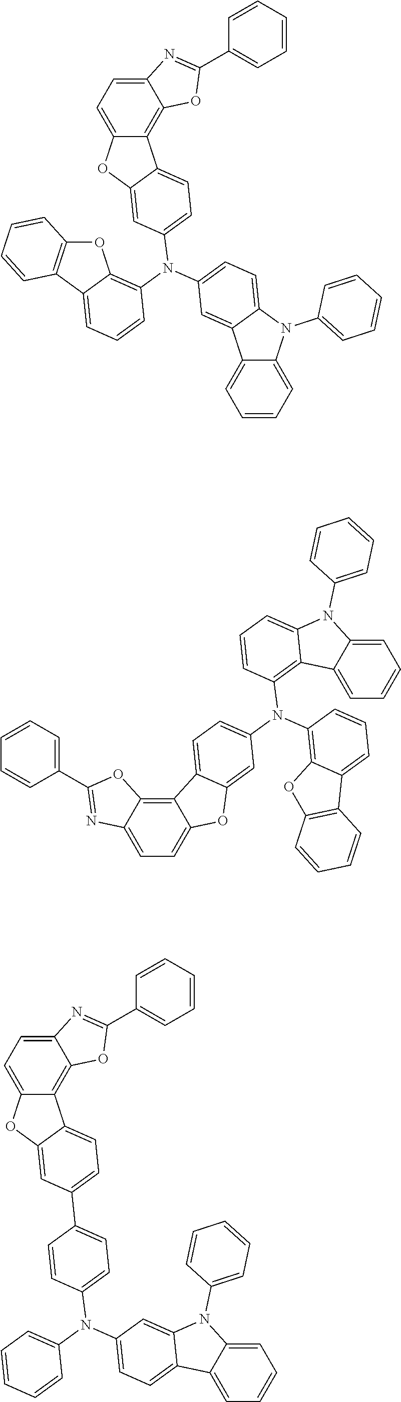 [SMILES: c1ccc(-c2nc3ccc4oc5cc(N(c6ccc7c(c6)c6ccccc6n7-c6ccccc6)c6cccc7c6oc6ccccc67)ccc5c4c3o2)cc1 ; c1ccc(-c2nc3ccc4oc5cc(N(c6cccc7c6oc6ccccc67)c6cccc7c6c6ccccc6n7-c6ccccc6)ccc5c4c3o2)cc1 ; c1ccc(-c2nc3ccc4oc5cc(-c6ccc(N(c7ccccc7)c7ccc8c9ccccc9n(-c9ccccc9)c8c7)cc6)ccc5c4c3o2)cc1]

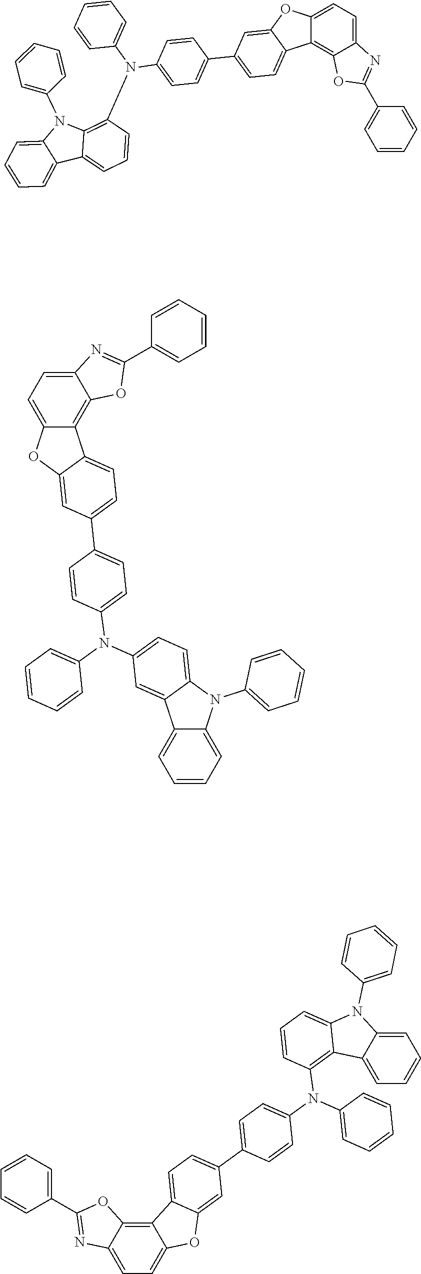 [SMILES: c1ccc(-c2nc3ccc4oc5cc(-c6ccc(N(c7ccccc7)c7cccc8c9ccccc9n(-c9ccccc9)c78)cc6)ccc5c4c3o2)cc1 ; c1ccc(-c2nc3ccc4oc5cc(-c6ccc(N(c7ccccc7)c7ccc8c(c7)c7ccccc7n8-c7ccccc7)cc6)ccc5c4c3o2)cc1 ; c1ccc(-c2nc3ccc4oc5cc(-c6ccc(N(c7ccccc7)c7cccc8c7c7ccccc7n8-c7ccccc7)cc6)ccc5c4c3o2)cc1]

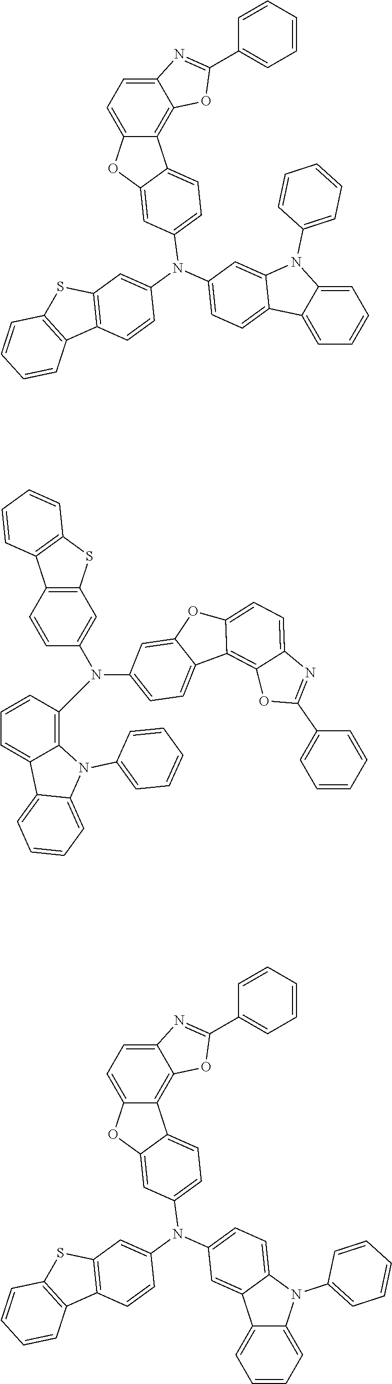 [SMILES: c1ccc(-c2nc3ccc4oc5cc(N(c6ccc7c(c6)sc6ccccc67)c6ccc7c8ccccc8n(-c8ccccc8)c7c6)ccc5c4c3o2)cc1 ; c1ccc(-c2nc3ccc4oc5cc(N(c6ccc7c(c6)sc6ccccc67)c6cccc7c8ccccc8n(-c8ccccc8)c67)ccc5c4c3o2)cc1 ; c1ccc(-c2nc3ccc4oc5cc(N(c6ccc7c(c6)sc6ccccc67)c6ccc7c(c6)c6ccccc6n7-c6ccccc6)ccc5c4c3o2)cc1]

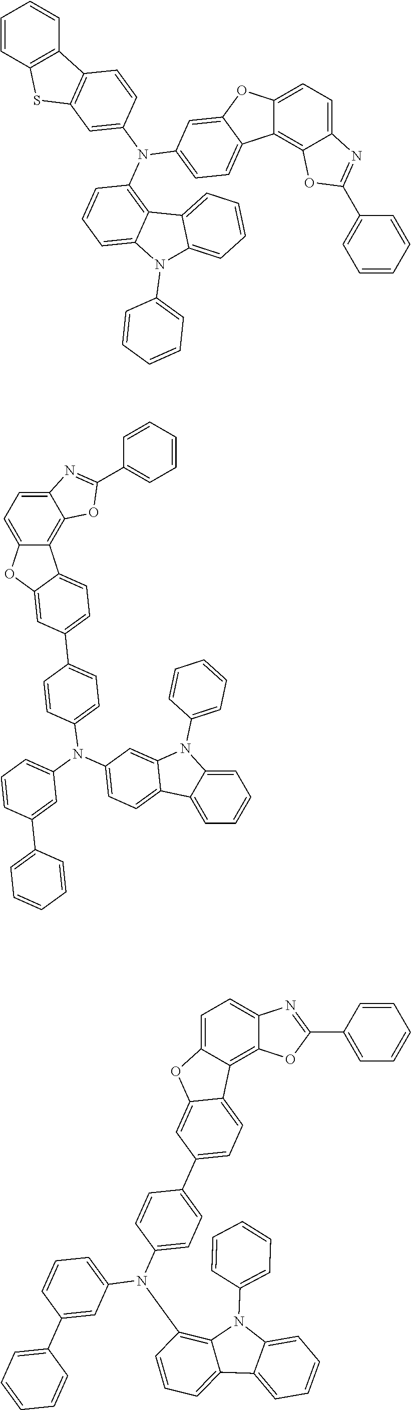 [SMILES: c1ccc(-c2nc3ccc4oc5cc(N(c6ccc7c(c6)sc6ccccc67)c6cccc7c6c6ccccc6n7-c6ccccc6)ccc5c4c3o2)cc1 ; c1ccc(-c2cccc(N(c3ccc(-c4ccc5c(c4)oc4ccc6nc(-c7ccccc7)oc6c45)cc3)c3ccc4c5ccccc5n(-c5ccccc5)c4c3)c2)cc1 ; c1ccc(-c2cccc(N(c3ccc(-c4ccc5c(c4)oc4ccc6nc(-c7ccccc7)oc6c45)cc3)c3cccc4c5ccccc5n(-c5ccccc5)c34)c2)cc1]

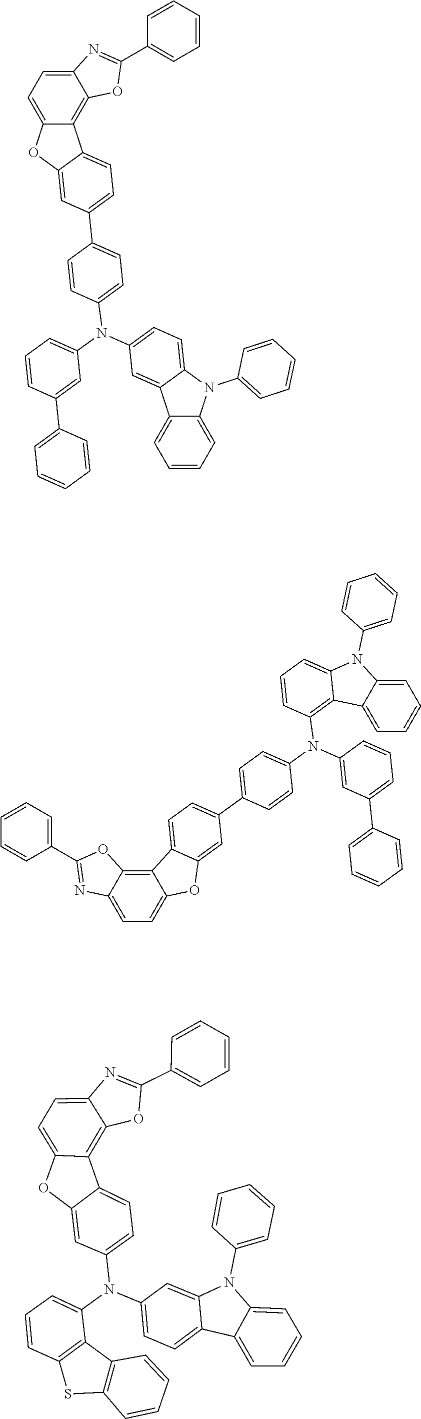 [SMILES: c1ccc(-c2cccc(N(c3ccc(-c4ccc5c(c4)oc4ccc6nc(-c7ccccc7)oc6c45)cc3)c3ccc4c(c3)c3ccccc3n4-c3ccccc3)c2)cc1 ; c1ccc(-c2cccc(N(c3ccc(-c4ccc5c(c4)oc4ccc6nc(-c7ccccc7)oc6c45)cc3)c3cccc4c3c3ccccc3n4-c3ccccc3)c2)cc1 ; c1ccc(-c2nc3ccc4oc5cc(N(c6ccc7c8ccccc8n(-c8ccccc8)c7c6)c6cccc7sc8ccccc8c67)ccc5c4c3o2)cc1]

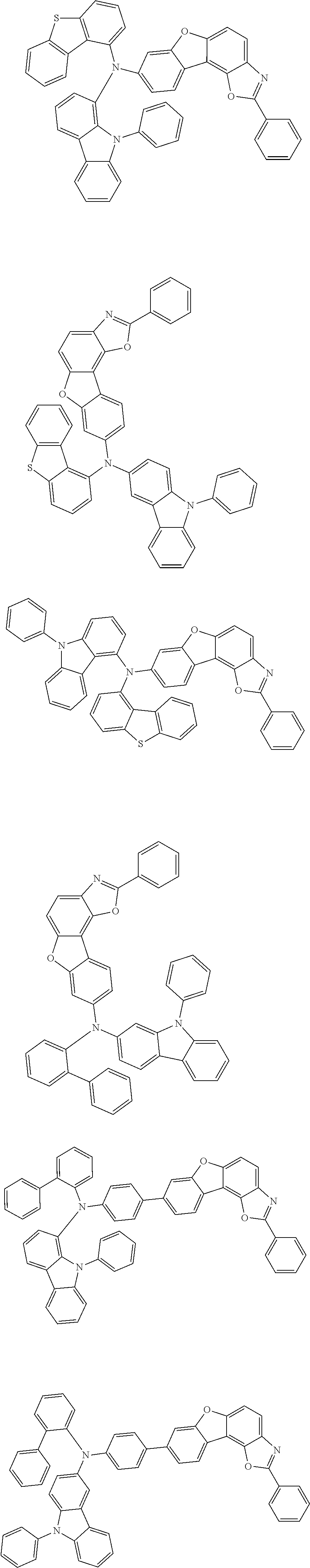

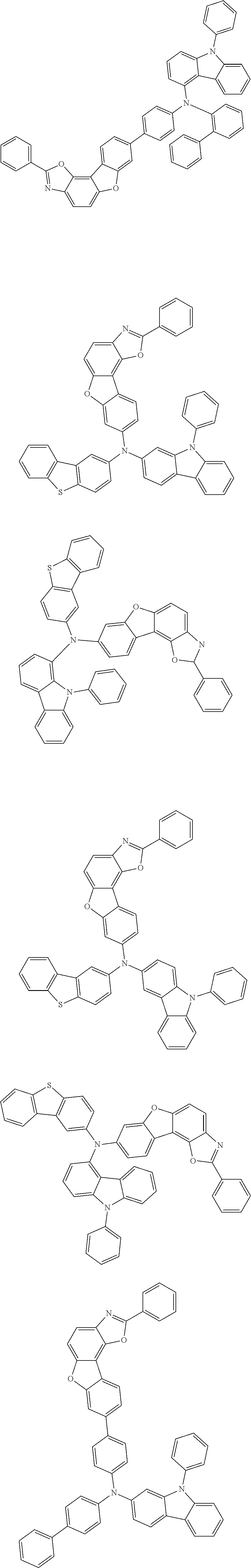

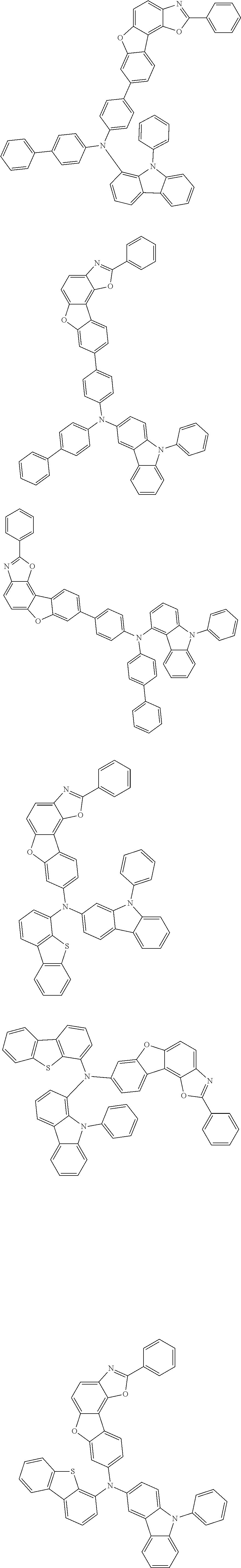

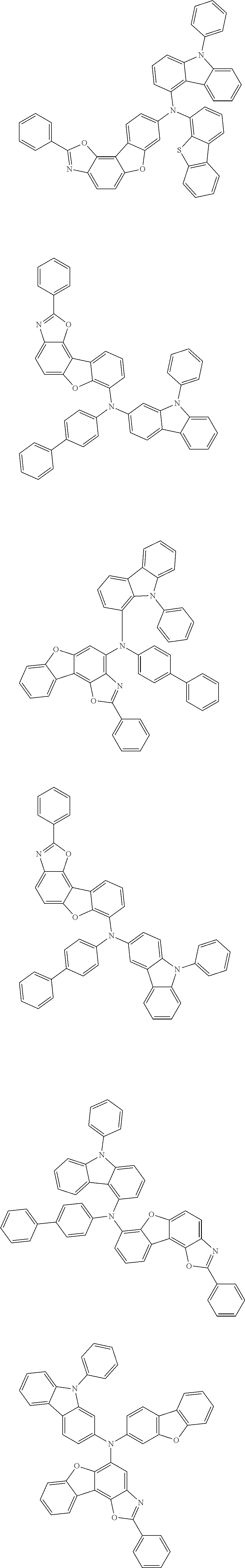

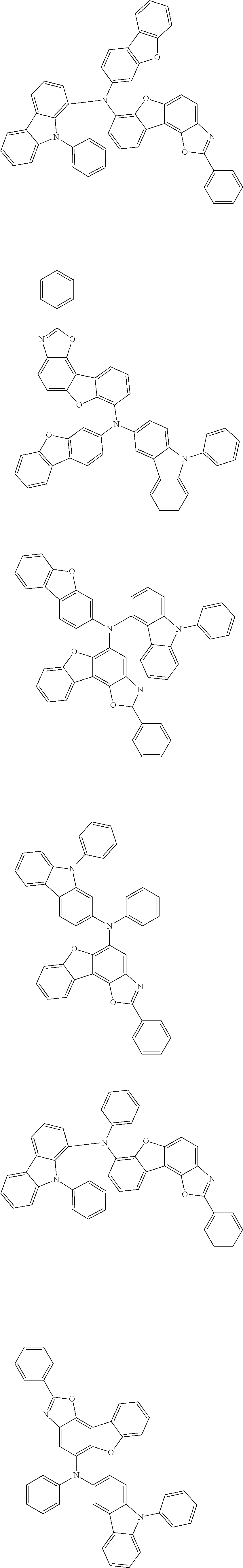

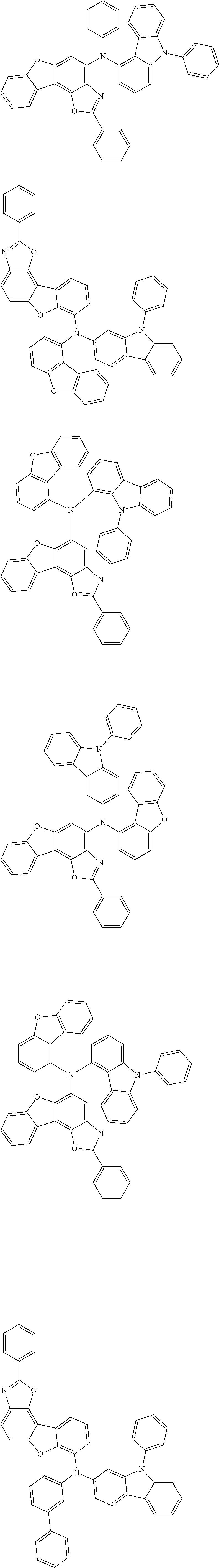

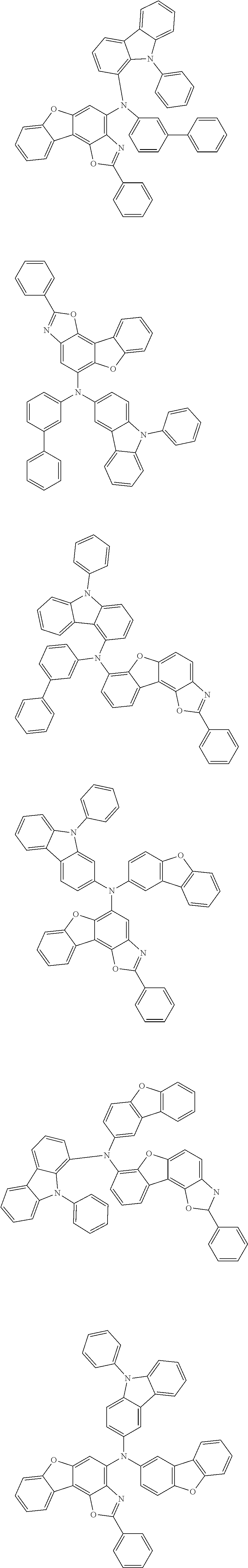

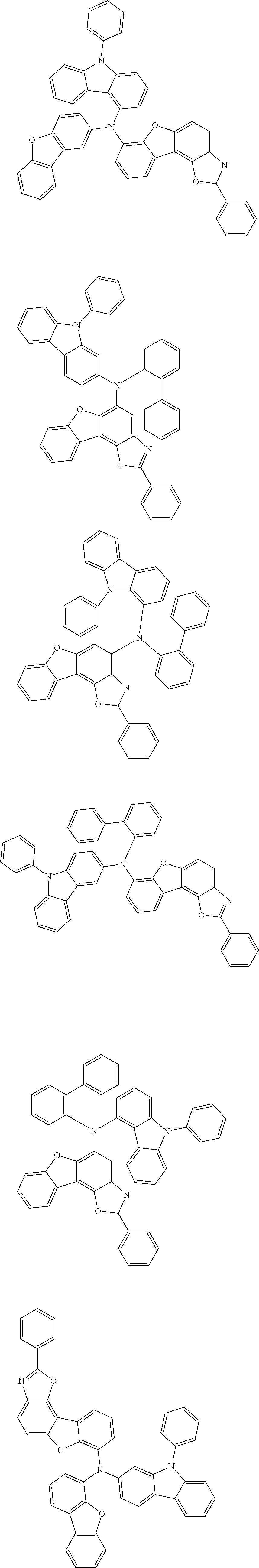

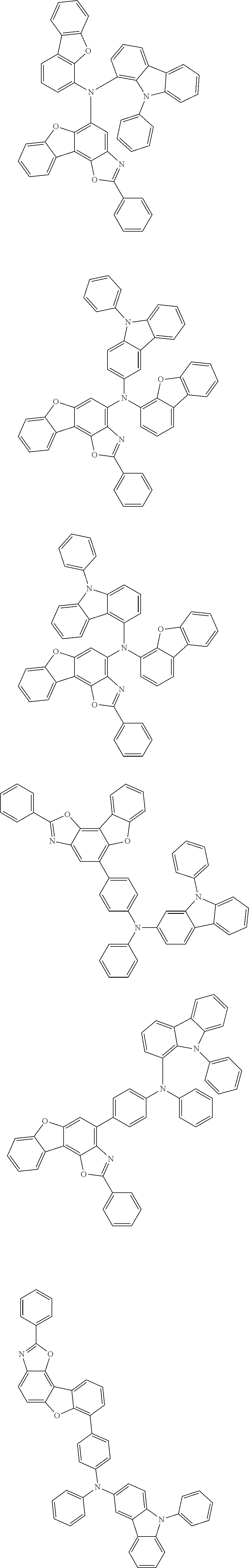

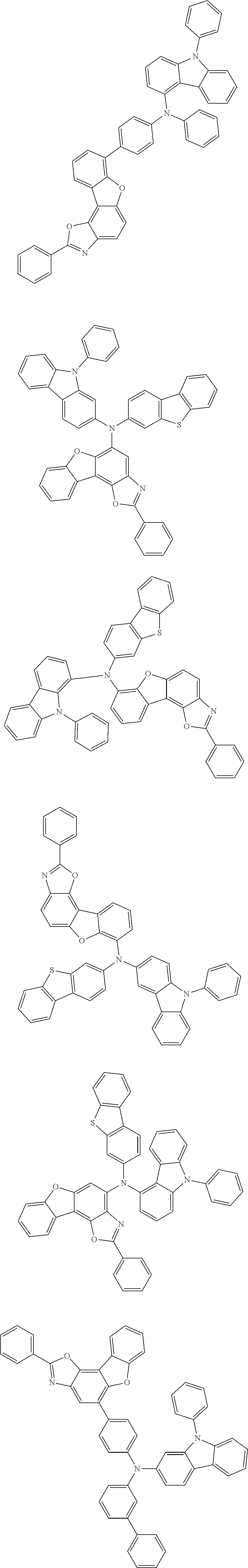

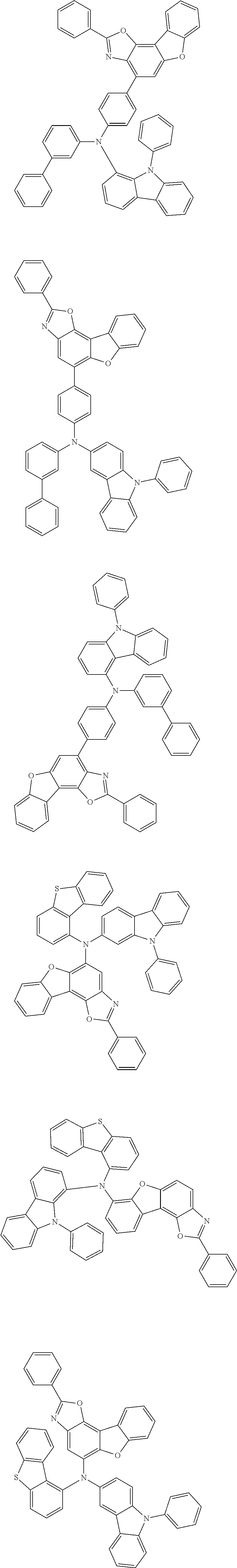

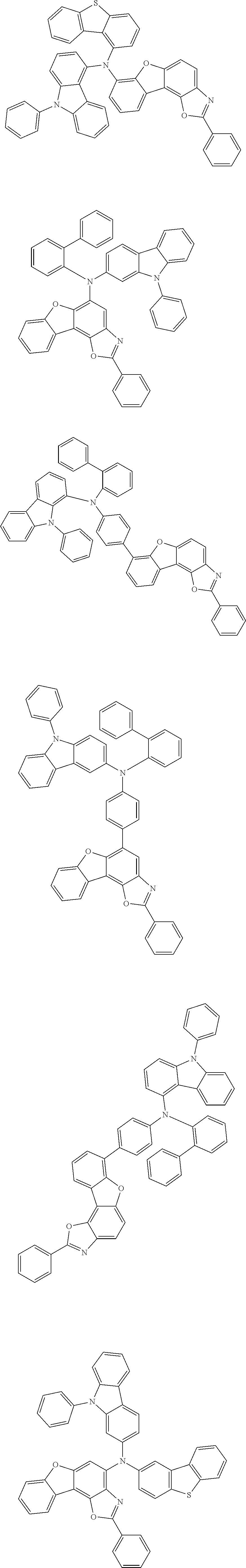

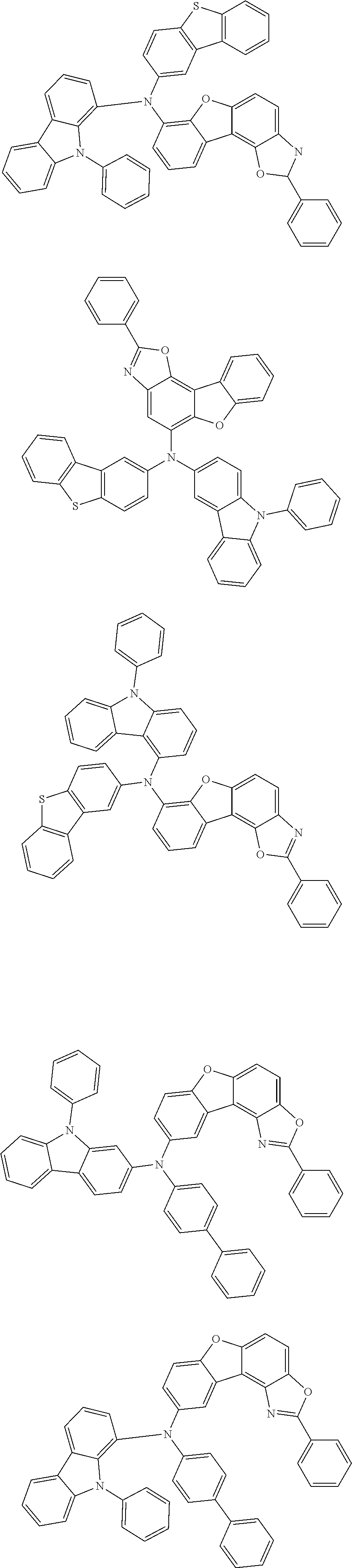

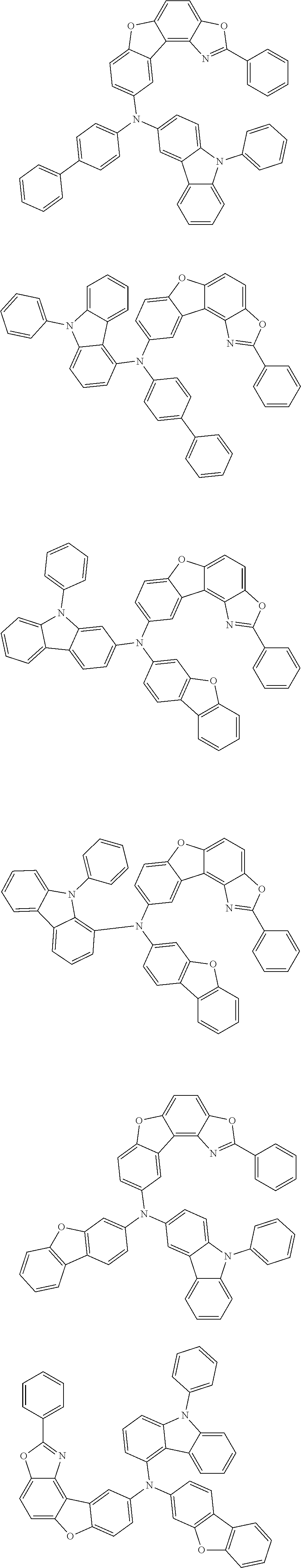

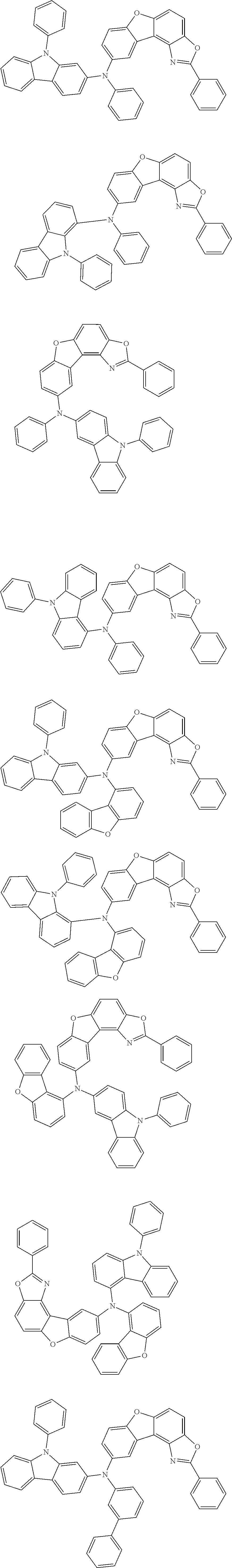

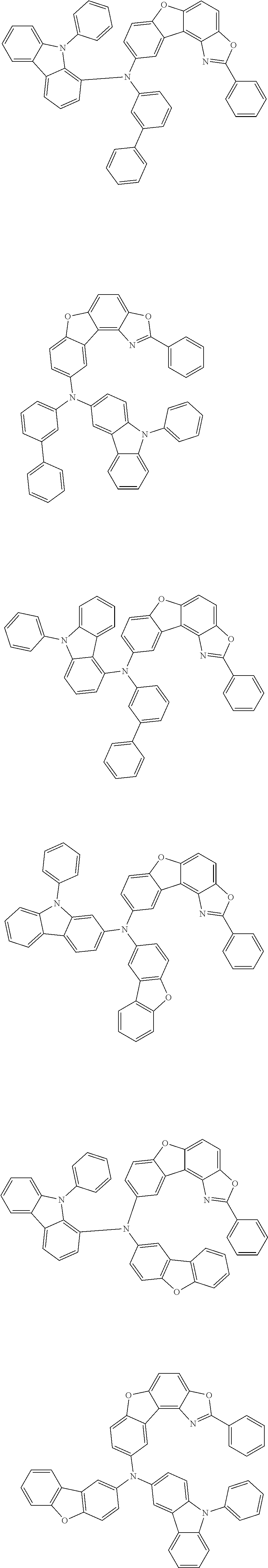

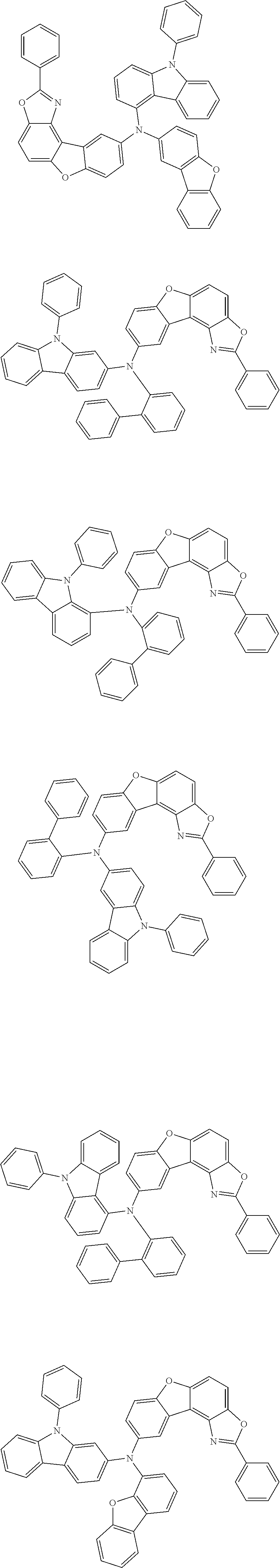

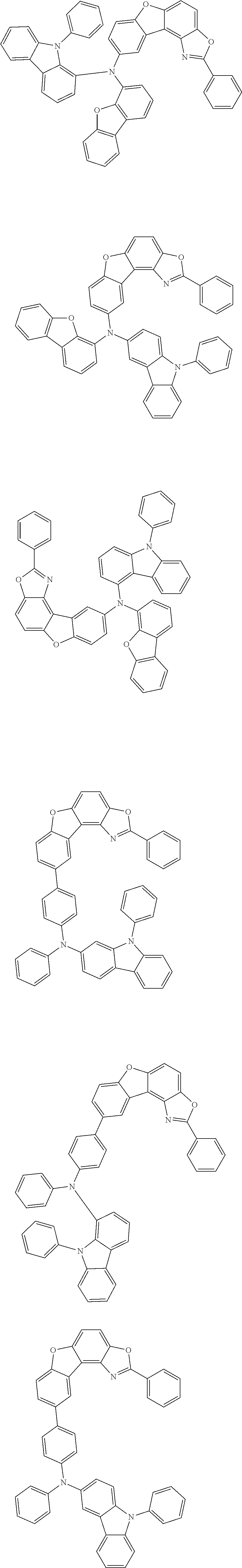

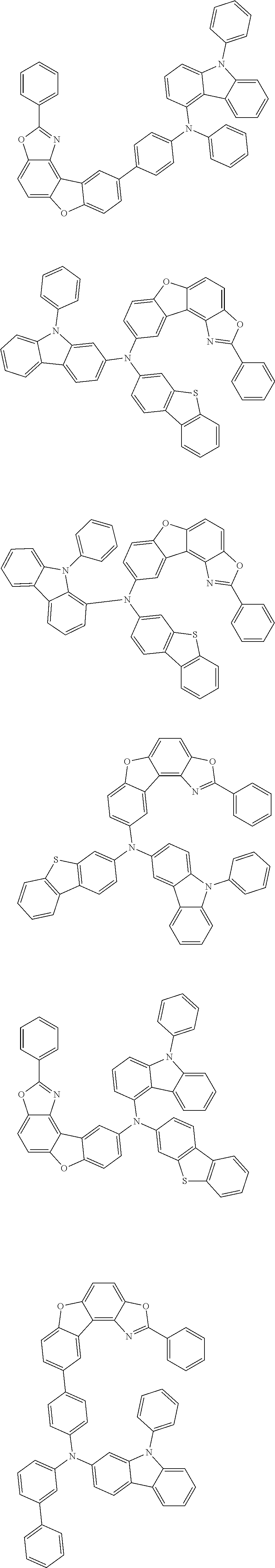

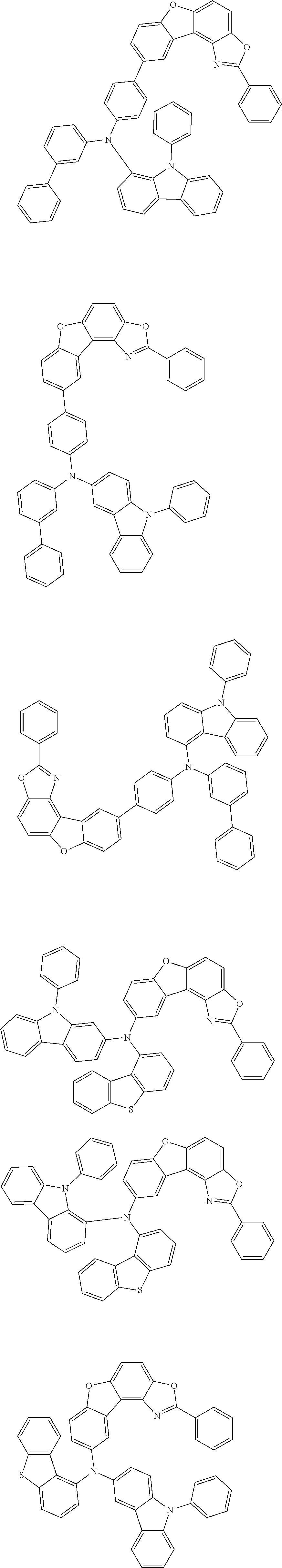

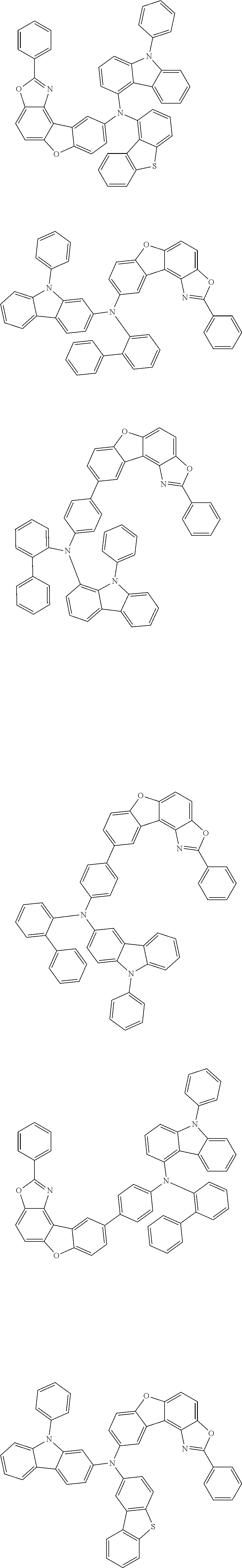

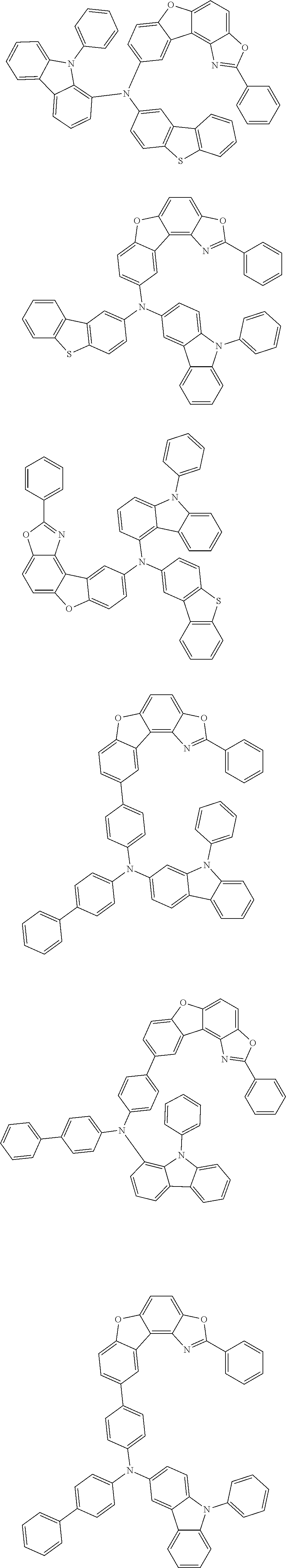

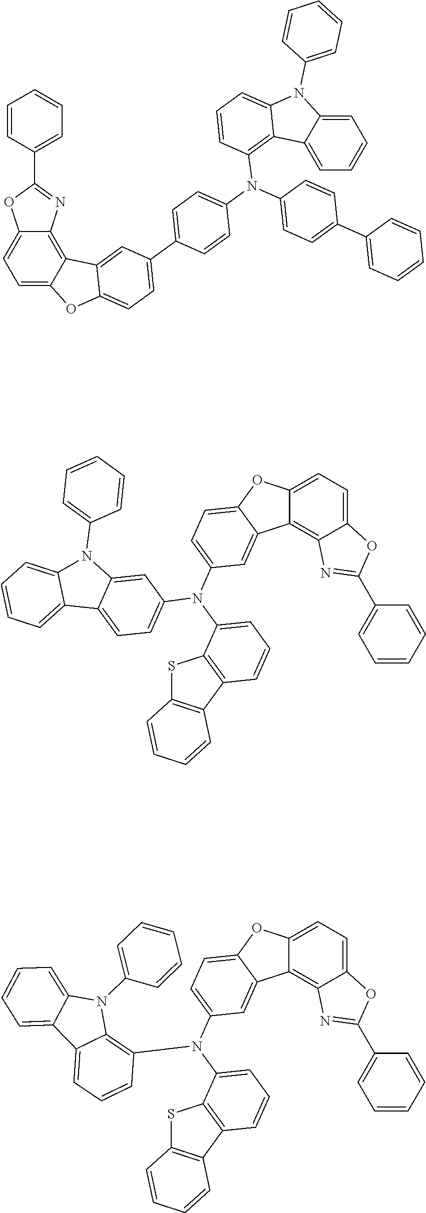 [SMILES: c1ccc(-c2ccc(N(c3ccc(-c4ccc5oc6ccc7oc(-c8ccccc8)nc7c6c5c4)cc3)c3cccc4c3c3ccccc3n4-c3ccccc3)cc2)cc1 ; c1ccc(-c2nc3c(ccc4oc5ccc(N(c6ccc7c8ccccc8n(-c8ccccc8)c7c6)c6cccc7c6sc6ccccc67)cc5c43)o2)cc1 ; c1ccc(-c2nc3c(ccc4oc5ccc(N(c6cccc7c6sc6ccccc67)c6cccc7c8ccccc8n(-c8ccccc8)c67)cc5c43)o2)cc1]

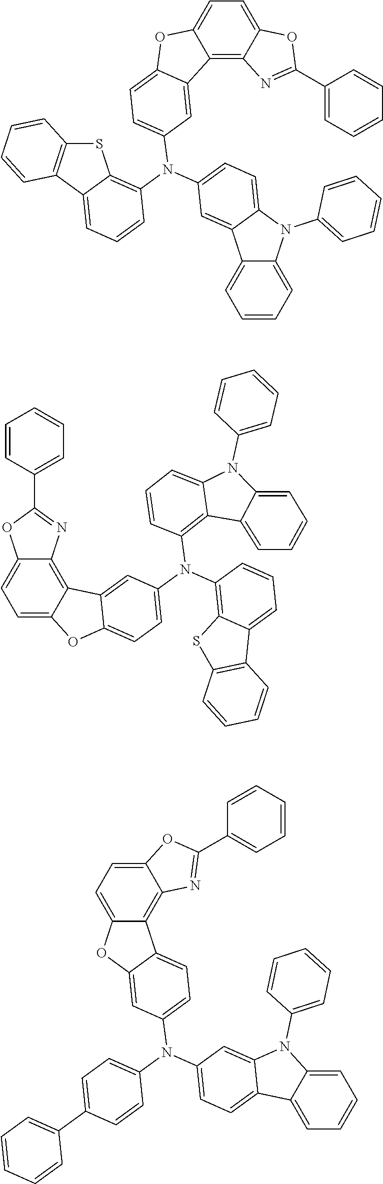 [SMILES: c1ccc(-c2nc3c(ccc4oc5ccc(N(c6ccc7c(c6)c6ccccc6n7-c6ccccc6)c6cccc7c6sc6ccccc67)cc5c43)o2)cc1 ; c1ccc(-c2nc3c(ccc4oc5ccc(N(c6cccc7c6sc6ccccc67)c6cccc7c6c6ccccc6n7-c6ccccc6)cc5c43)o2)cc1 ; c1ccc(-c2ccc(N(c3ccc4c(c3)oc3ccc5oc(-c6ccccc6)nc5c34)c3ccc4c5ccccc5n(-c5ccccc5)c4c3)cc2)cc1]

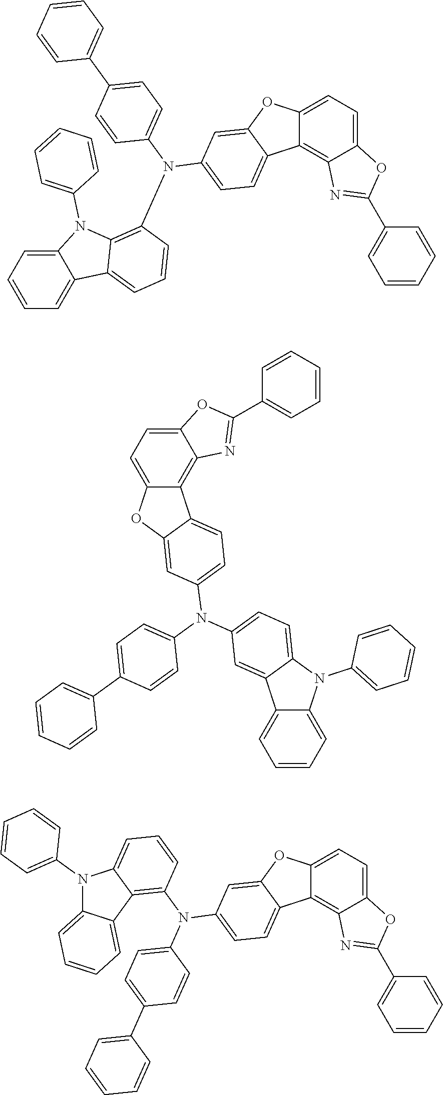 [SMILES: c1ccc(-c2ccc(N(c3ccc4c(c3)oc3ccc5oc(-c6ccccc6)nc5c34)c3cccc4c5ccccc5n(-c5ccccc5)c34)cc2)cc1 ; c1ccc(-c2ccc(N(c3ccc4c(c3)oc3ccc5oc(-c6ccccc6)nc5c34)c3ccc4c(c3)c3ccccc3n4-c3ccccc3)cc2)cc1 ; c1ccc(-c2ccc(N(c3ccc4c(c3)oc3ccc5oc(-c6ccccc6)nc5c34)c3cccc4c3c3ccccc3n4-c3ccccc3)cc2)cc1]

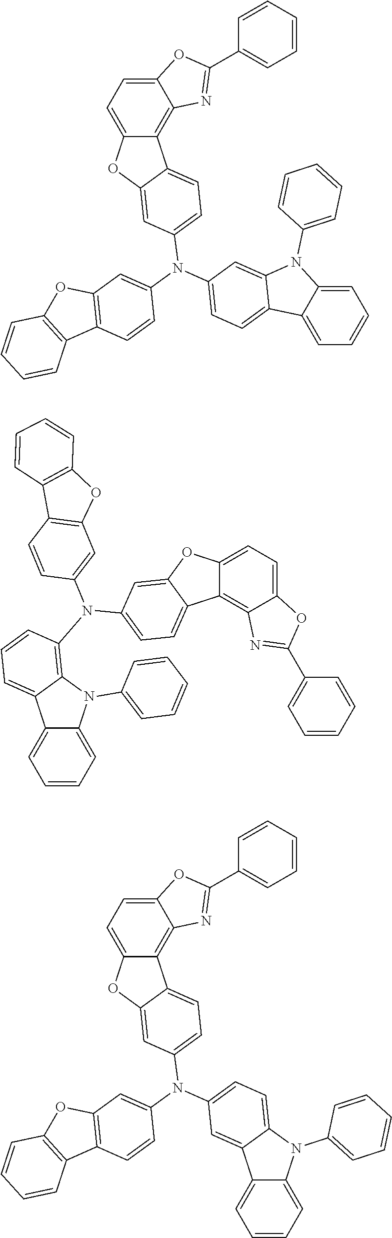

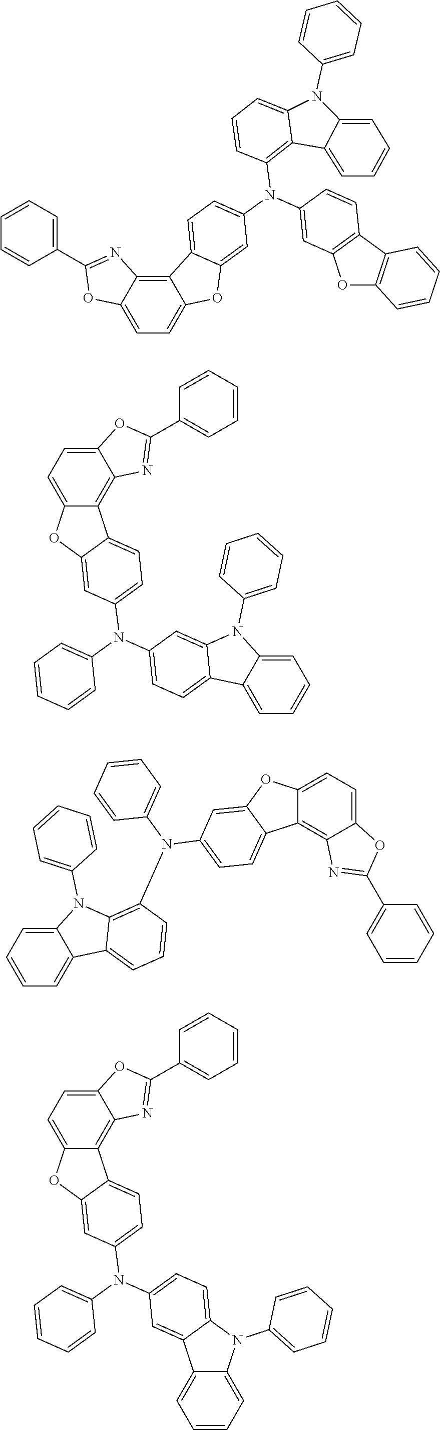 [SMILES: c1ccc(-c2nc3c(ccc4oc5cc(N(c6ccc7c(c6)oc6ccccc67)c6cccc7c6c6ccccc6n7-c6ccccc6)ccc5c43)o2)cc1 ; c1ccc(-c2nc3c(ccc4oc5cc(N(c6ccccc6)c6ccc7c8ccccc8n(-c8ccccc8)c7c6)ccc5c43)o2)cc1 ; c1ccc(-c2nc3c(ccc4oc5cc(N(c6ccccc6)c6cccc7c8ccccc8n(-c8ccccc8)c67)ccc5c43)o2)cc1 ; c1ccc(-c2nc3c(ccc4oc5cc(N(c6ccccc6)c6ccc7c(c6)c6ccccc6n7-c6ccccc6)ccc5c43)o2)cc1]

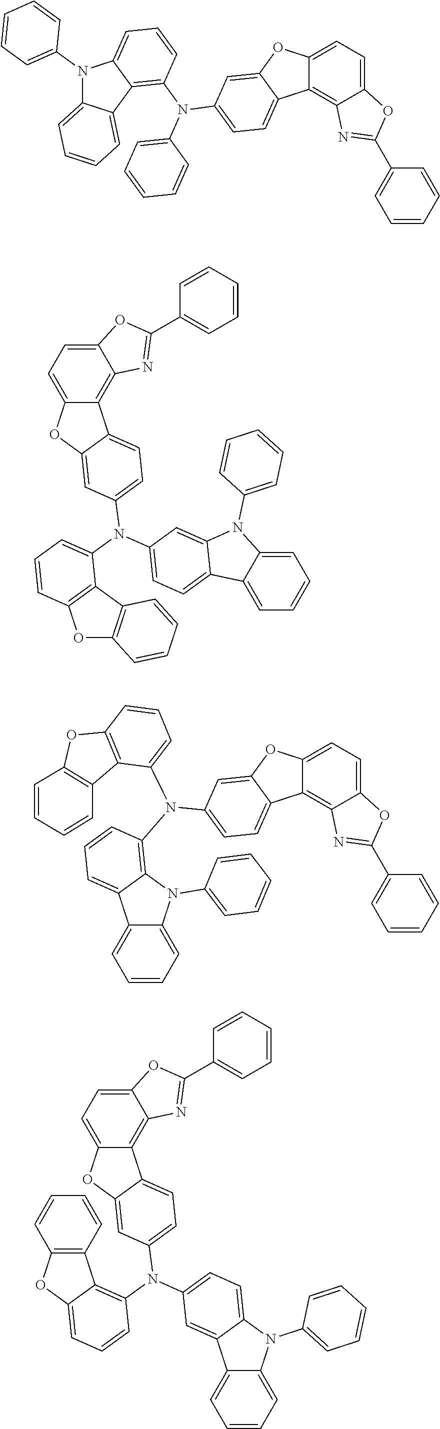

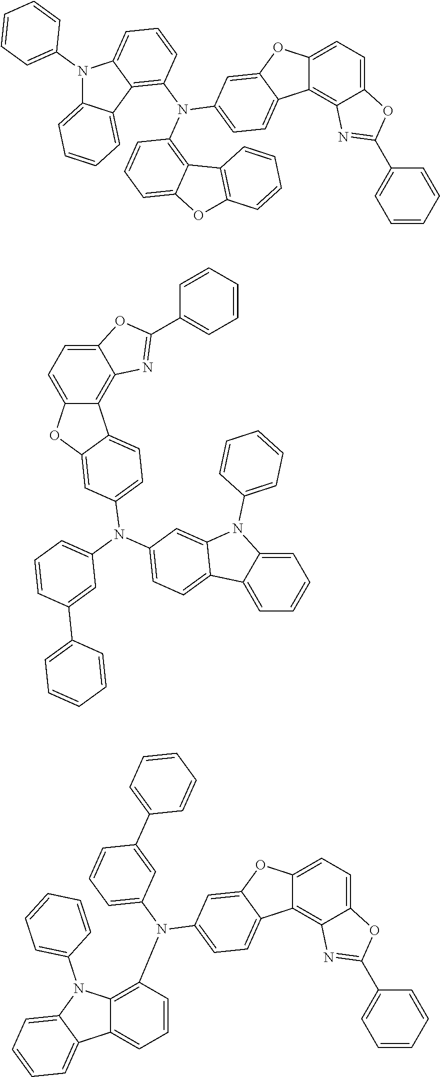 [SMILES: c1ccc(-c2nc3c(ccc4oc5cc(N(c6cccc7oc8ccccc8c67)c6cccc7c6c6ccccc6n7-c6ccccc6)ccc5c43)o2)cc1 ; c1ccc(-c2cccc(N(c3ccc4c(c3)oc3ccc5oc(-c6ccccc6)nc5c34)c3ccc4c5ccccc5n(-c5ccccc5)c4c3)c2)cc1 ; c1ccc(-c2cccc(N(c3ccc4c(c3)oc3ccc5oc(-c6ccccc6)nc5c34)c3cccc4c5ccccc5n(-c5ccccc5)c34)c2)cc1]

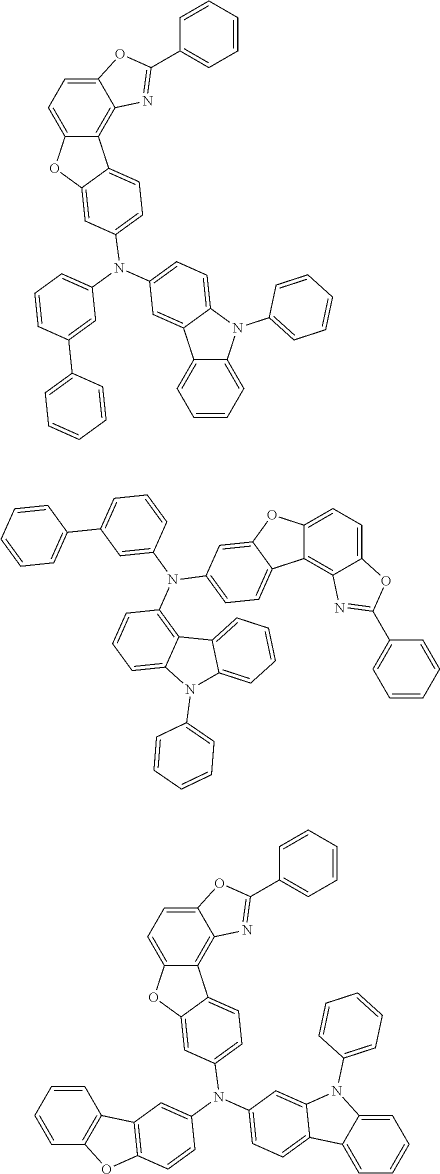 [SMILES: c1ccc(-c2cccc(N(c3ccc4c(c3)oc3ccc5oc(-c6ccccc6)nc5c34)c3ccc4c(c3)c3ccccc3n4-c3ccccc3)c2)cc1 ; c1ccc(-c2cccc(N(c3ccc4c(c3)oc3ccc5oc(-c6ccccc6)nc5c34)c3cccc4c3c3ccccc3n4-c3ccccc3)c2)cc1 ; c1ccc(-c2nc3c(ccc4oc5cc(N(c6ccc7oc8ccccc8c7c6)c6ccc7c8ccccc8n(-c8ccccc8)c7c6)ccc5c43)o2)cc1]

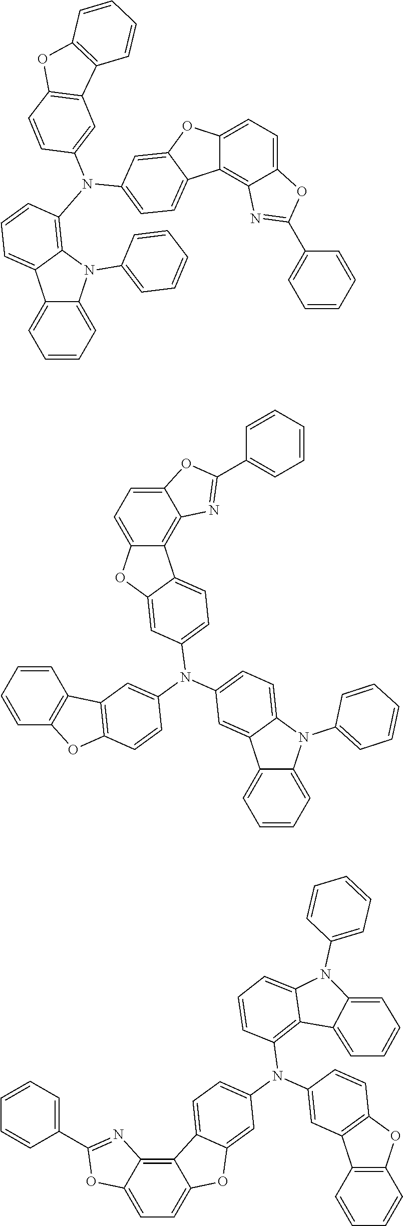 [SMILES: c1ccc(-c2nc3c(ccc4oc5cc(N(c6ccc7oc8ccccc8c7c6)c6cccc7c8ccccc8n(-c8ccccc8)c67)ccc5c43)o2)cc1 ; c1ccc(-c2nc3c(ccc4oc5cc(N(c6ccc7oc8ccccc8c7c6)c6ccc7c(c6)c6ccccc6n7-c6ccccc6)ccc5c43)o2)cc1 ; c1ccc(-c2nc3c(ccc4oc5cc(N(c6ccc7oc8ccccc8c7c6)c6cccc7c6c6ccccc6n7-c6ccccc6)ccc5c43)o2)cc1]

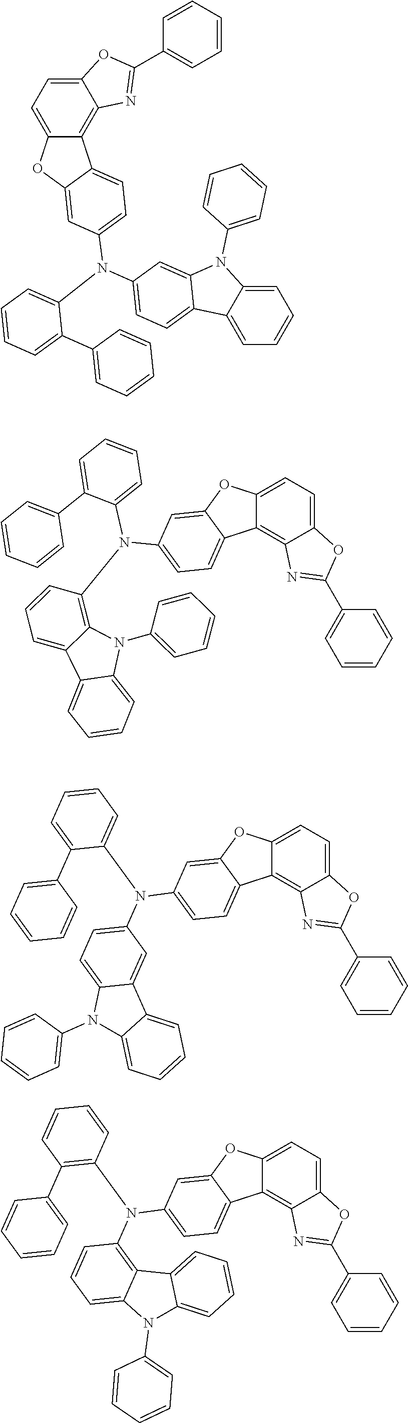

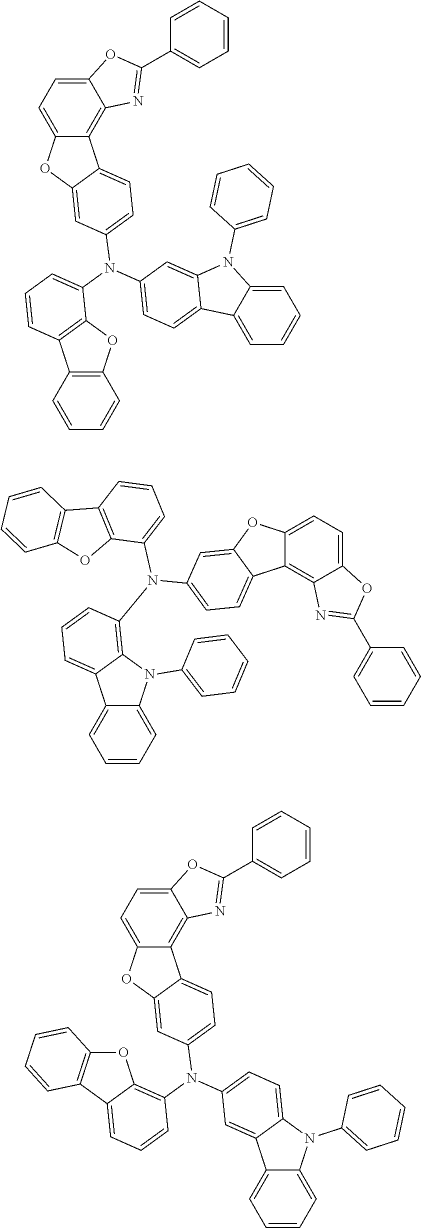 [SMILES: c1ccc(-c2nc3c(ccc4oc5cc(N(c6ccc7c8ccccc8n(-c8ccccc8)c7c6)c6cccc7c6oc6ccccc67)ccc5c43)o2)cc1 ; c1ccc(-c2nc3c(ccc4oc5cc(N(c6cccc7c6oc6ccccc67)c6cccc7c8ccccc8n(-c8ccccc8)c67)ccc5c43)o2)cc1 ; c1ccc(-c2nc3c(ccc4oc5cc(N(c6ccc7c(c6)c6ccccc6n7-c6ccccc6)c6cccc7c6oc6ccccc67)ccc5c43)o2)cc1]

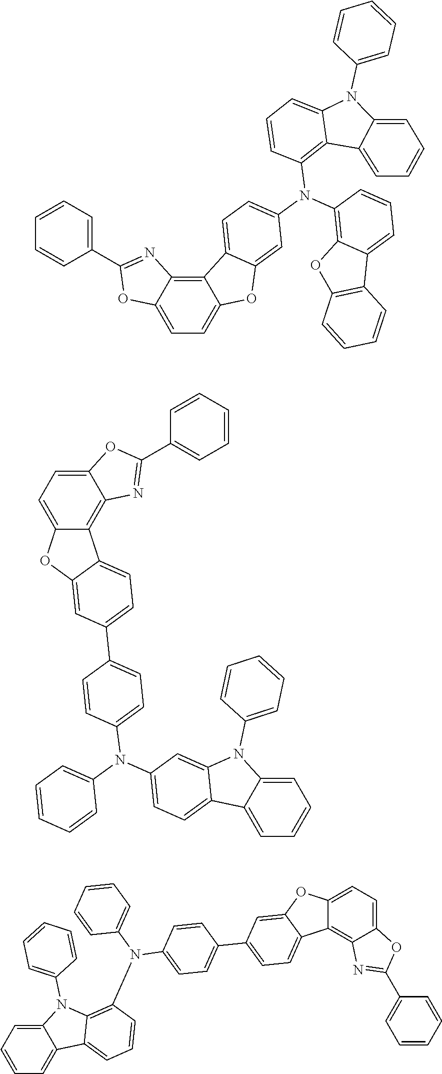 [SMILES: c1ccc(-c2nc3c(ccc4oc5cc(N(c6cccc7c6oc6ccccc67)c6cccc7c6c6ccccc6n7-c6ccccc6)ccc5c43)o2)cc1 ; c1ccc(-c2nc3c(ccc4oc5cc(-c6ccc(N(c7ccccc7)c7ccc8c9ccccc9n(-c9ccccc9)c8c7)cc6)ccc5c43)o2)cc1 ; c1ccc(-c2nc3c(ccc4oc5cc(-c6ccc(N(c7ccccc7)c7cccc8c9ccccc9n(-c9ccccc9)c78)cc6)ccc5c43)o2)cc1]

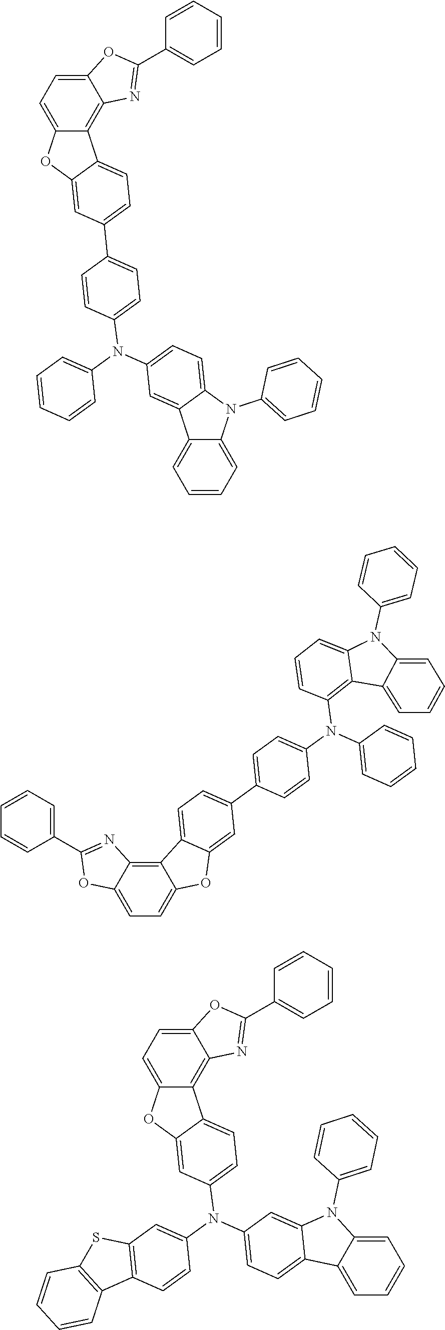 [SMILES: c1ccc(-c2nc3c(ccc4oc5cc(-c6ccc(N(c7ccccc7)c7ccc8c(c7)c7ccccc7n8-c7ccccc7)cc6)ccc5c43)o2)cc1 ; c1ccc(-c2nc3c(ccc4oc5cc(-c6ccc(N(c7ccccc7)c7cccc8c7c7ccccc7n8-c7ccccc7)cc6)ccc5c43)o2)cc1 ; c1ccc(-c2nc3c(ccc4oc5cc(N(c6ccc7c(c6)sc6ccccc67)c6ccc7c8ccccc8n(-c8ccccc8)c7c6)ccc5c43)o2)cc1]

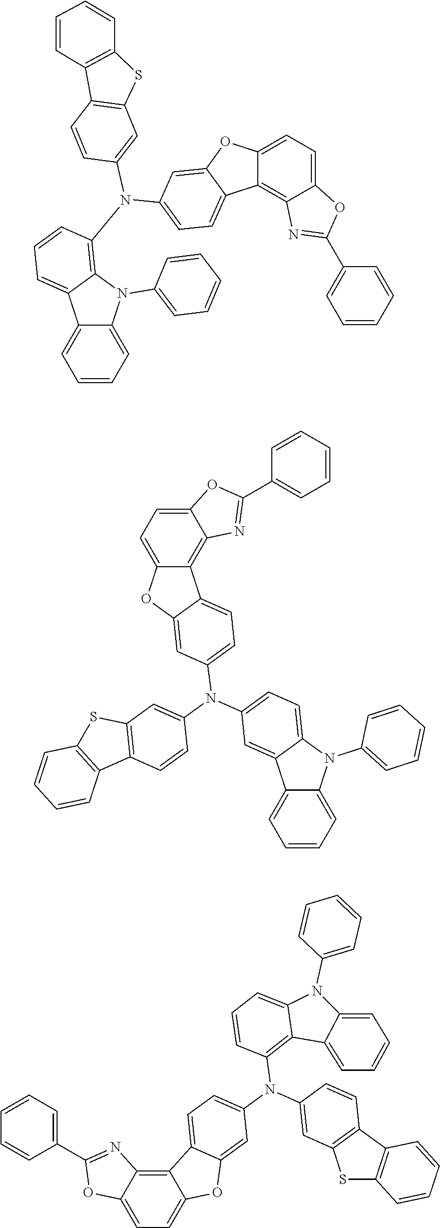 [SMILES: c1ccc(-c2nc3c(ccc4oc5cc(N(c6ccc7c(c6)sc6ccccc67)c6cccc7c8ccccc8n(-c8ccccc8)c67)ccc5c43)o2)cc1 ; c1ccc(-c2nc3c(ccc4oc5cc(N(c6ccc7c(c6)sc6ccccc67)c6ccc7c(c6)c6ccccc6n7-c6ccccc6)ccc5c43)o2)cc1 ; c1ccc(-c2nc3c(ccc4oc5cc(N(c6ccc7c(c6)sc6ccccc67)c6cccc7c6c6ccccc6n7-c6ccccc6)ccc5c43)o2)cc1]

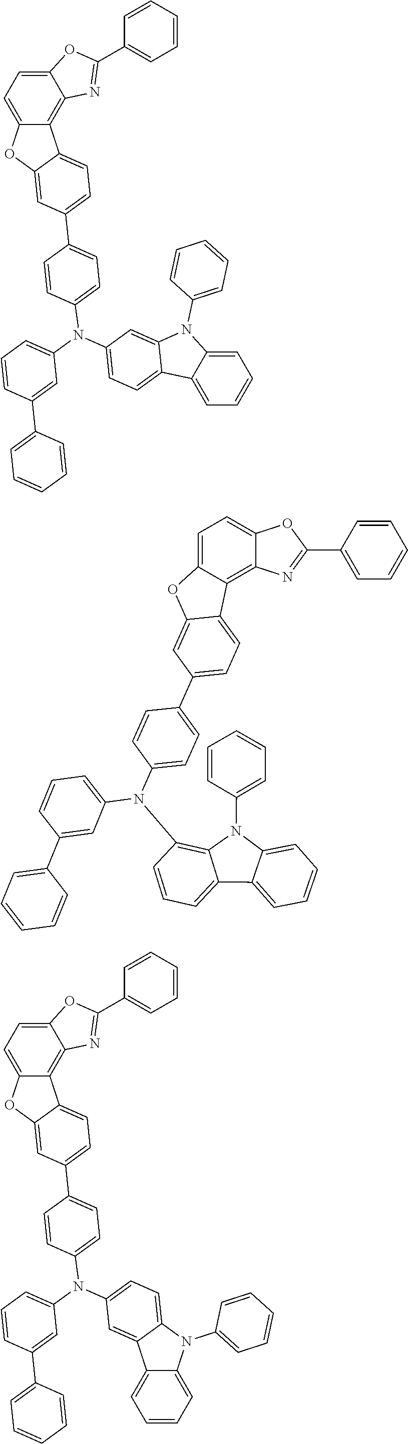 [SMILES: c1ccc(-c2cccc(N(c3ccc(-c4ccc5c(c4)oc4ccc6oc(-c7ccccc7)nc6c45)cc3)c3ccc4c5ccccc5n(-c5ccccc5)c4c3)c2)cc1 ; c1ccc(-c2cccc(N(c3ccc(-c4ccc5c(c4)oc4ccc6oc(-c7ccccc7)nc6c45)cc3)c3cccc4c5ccccc5n(-c5ccccc5)c34)c2)cc1 ; c1ccc(-c2cccc(N(c3ccc(-c4ccc5c(c4)oc4ccc6oc(-c7ccccc7)nc6c45)cc3)c3ccc4c(c3)c3ccccc3n4-c3ccccc3)c2)cc1]

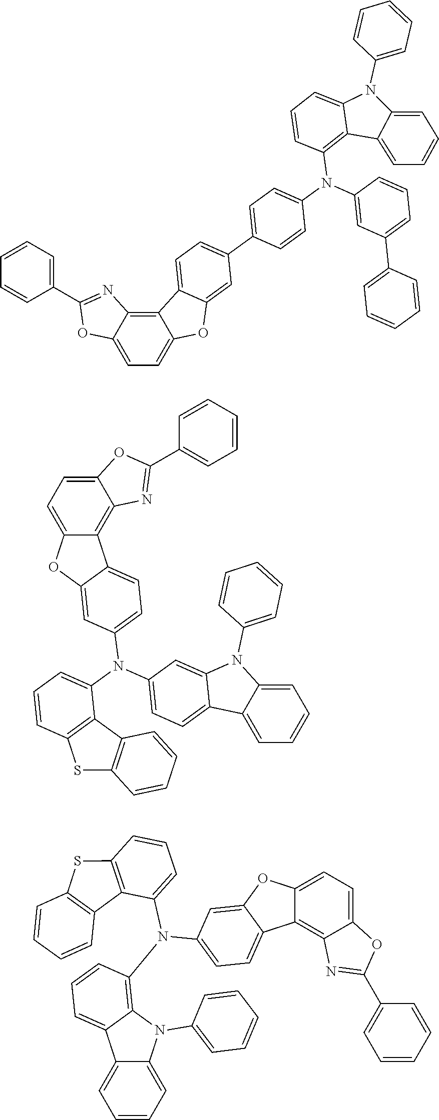

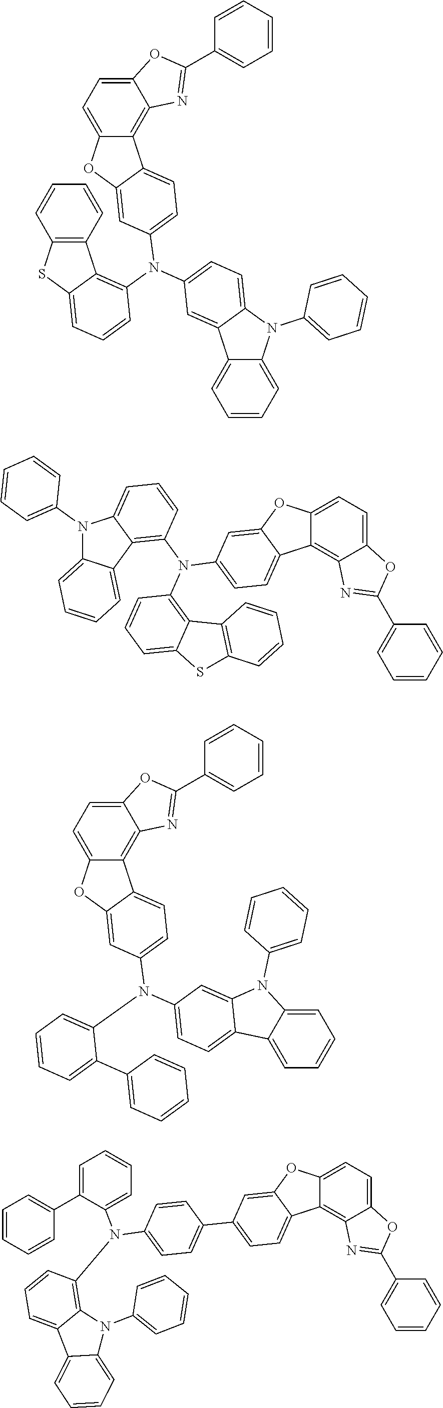 [SMILES: c1ccc(-c2nc3c(ccc4oc5cc(N(c6ccc7c(c6)c6ccccc6n7-c6ccccc6)c6cccc7sc8ccccc8c67)ccc5c43)o2)cc1 ; c1ccc(-c2nc3c(ccc4oc5cc(N(c6cccc7sc8ccccc8c67)c6cccc7c6c6ccccc6n7-c6ccccc6)ccc5c43)o2)cc1 ; c1ccc(-c2nc3c(ccc4oc5cc(N(c6ccc7c8ccccc8n(-c8ccccc8)c7c6)c6ccccc6-c6ccccc6)ccc5c43)o2)cc1 ; c1ccc(-c2nc3c(ccc4oc5cc(-c6ccc(N(c7ccccc7-c7ccccc7)c7cccc8c9ccccc9n(-c9ccccc9)c78)cc6)ccc5c43)o2)cc1]

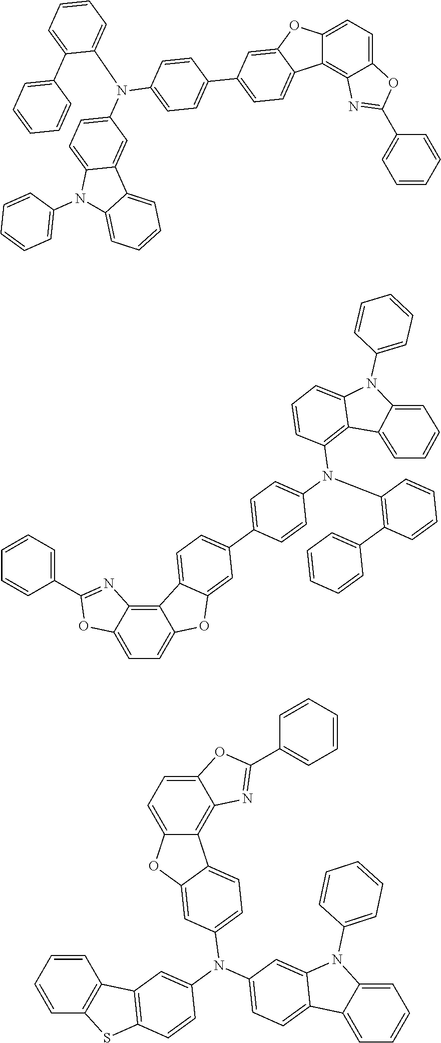 [SMILES: c1ccc(-c2nc3c(ccc4oc5cc(-c6ccc(N(c7ccc8c(c7)c7ccccc7n8-c7ccccc7)c7ccccc7-c7ccccc7)cc6)ccc5c43)o2)cc1 ; c1ccc(-c2nc3c(ccc4oc5cc(-c6ccc(N(c7ccccc7-c7ccccc7)c7cccc8c7c7ccccc7n8-c7ccccc7)cc6)ccc5c43)o2)cc1 ; c1ccc(-c2nc3c(ccc4oc5cc(N(c6ccc7sc8ccccc8c7c6)c6ccc7c8ccccc8n(-c8ccccc8)c7c6)ccc5c43)o2)cc1]

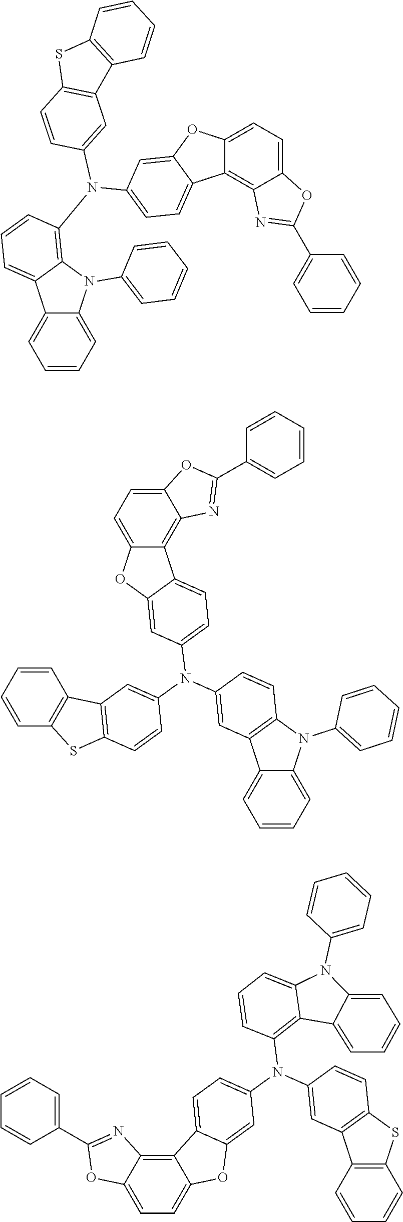 [SMILES: c1ccc(-c2nc3c(ccc4oc5cc(N(c6ccc7sc8ccccc8c7c6)c6cccc7c8ccccc8n(-c8ccccc8)c67)ccc5c43)o2)cc1 ; c1ccc(-c2nc3c(ccc4oc5cc(N(c6ccc7sc8ccccc8c7c6)c6ccc7c(c6)c6ccccc6n7-c6ccccc6)ccc5c43)o2)cc1 ; c1ccc(-c2nc3c(ccc4oc5cc(N(c6ccc7sc8ccccc8c7c6)c6cccc7c6c6ccccc6n7-c6ccccc6)ccc5c43)o2)cc1]

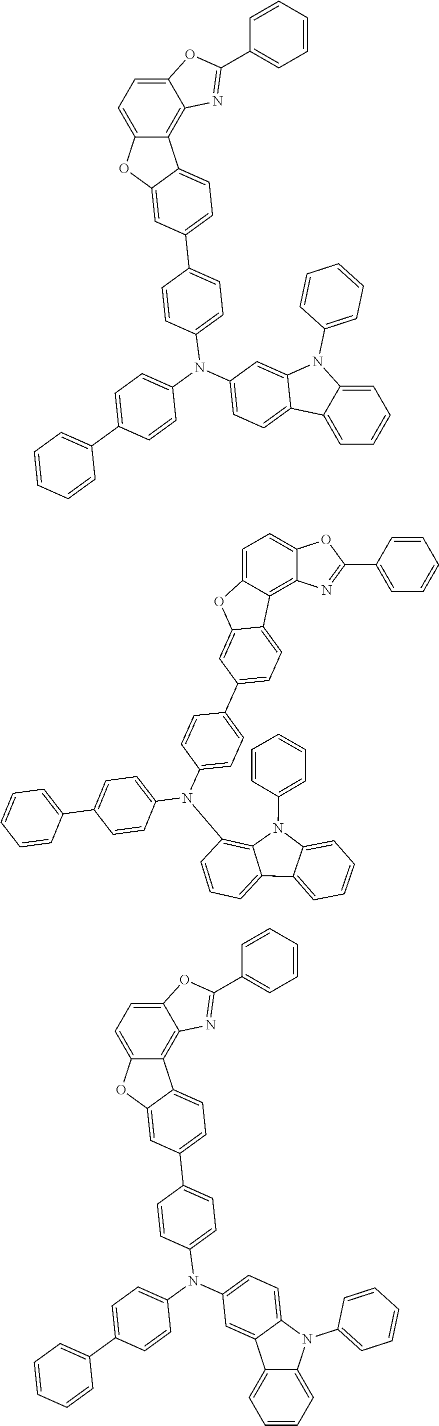

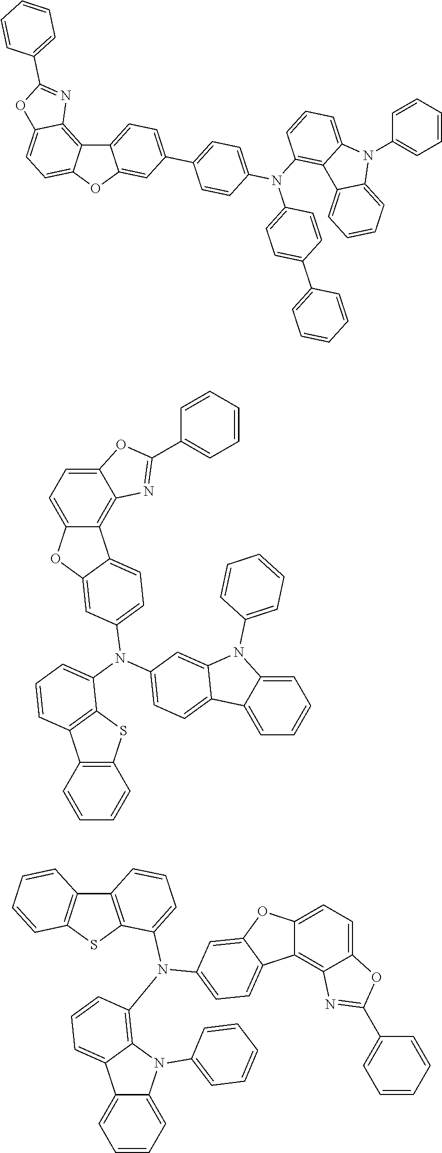 [SMILES: c1ccc(-c2ccc(N(c3ccc(-c4ccc5c(c4)oc4ccc6oc(-c7ccccc7)nc6c45)cc3)c3cccc4c3c3ccccc3n4-c3ccccc3)cc2)cc1 ; c1ccc(-c2nc3c(ccc4oc5cc(N(c6ccc7c8ccccc8n(-c8ccccc8)c7c6)c6cccc7c6sc6ccccc67)ccc5c43)o2)cc1 ; c1ccc(-c2nc3c(ccc4oc5cc(N(c6cccc7c6sc6ccccc67)c6cccc7c8ccccc8n(-c8ccccc8)c67)ccc5c43)o2)cc1]

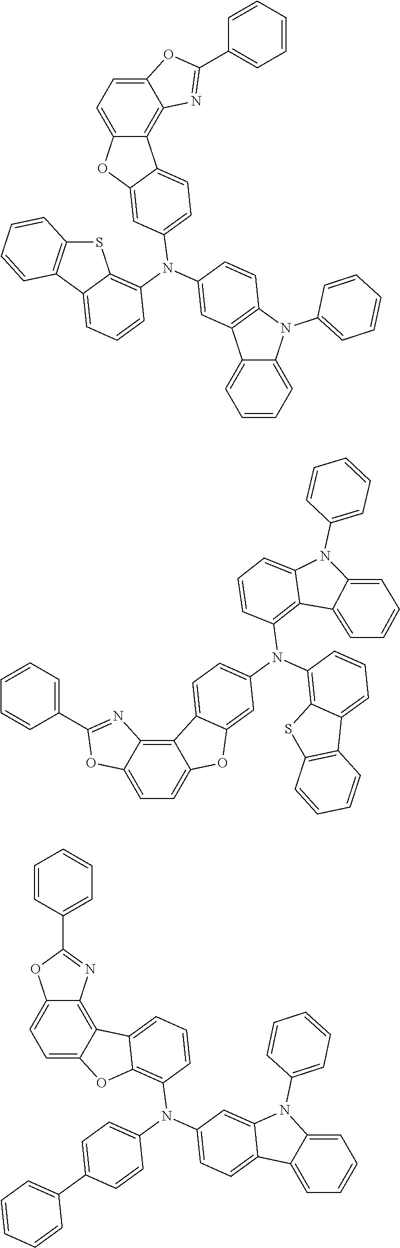 [SMILES: c1ccc(-c2nc3c(ccc4oc5cc(N(c6ccc7c(c6)c6ccccc6n7-c6ccccc6)c6cccc7c6sc6ccccc67)ccc5c43)o2)cc1 ; c1ccc(-c2nc3c(ccc4oc5cc(N(c6cccc7c6sc6ccccc67)c6cccc7c6c6ccccc6n7-c6ccccc6)ccc5c43)o2)cc1 ; c1ccc(-c2ccc(N(c3ccc4c5ccccc5n(-c5ccccc5)c4c3)c3cccc4c3oc3ccc5oc(-c6ccccc6)nc5c34)cc2)cc1]

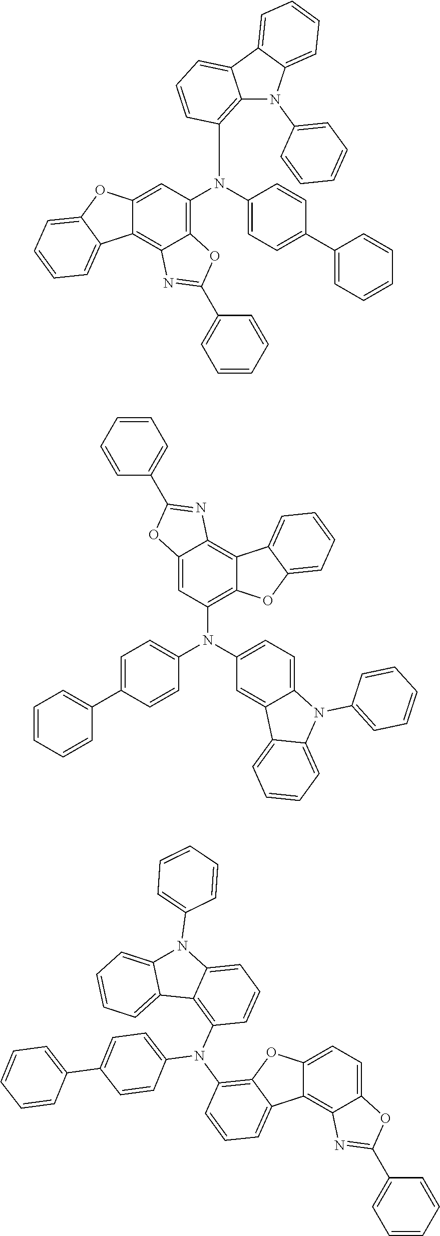 [SMILES: c1ccc(-c2ccc(N(c3cc4oc5ccccc5c4c4nc(-c5ccccc5)oc34)c3cccc4c5ccccc5n(-c5ccccc5)c34)cc2)cc1 ; c1ccc(-c2ccc(N(c3ccc4c(c3)c3ccccc3n4-c3ccccc3)c3cc4oc(-c5ccccc5)nc4c4c3oc3ccccc34)cc2)cc1 ; c1ccc(-c2ccc(N(c3cccc4c3oc3ccc5oc(-c6ccccc6)nc5c34)c3cccc4c3c3ccccc3n4-c3ccccc3)cc2)cc1]

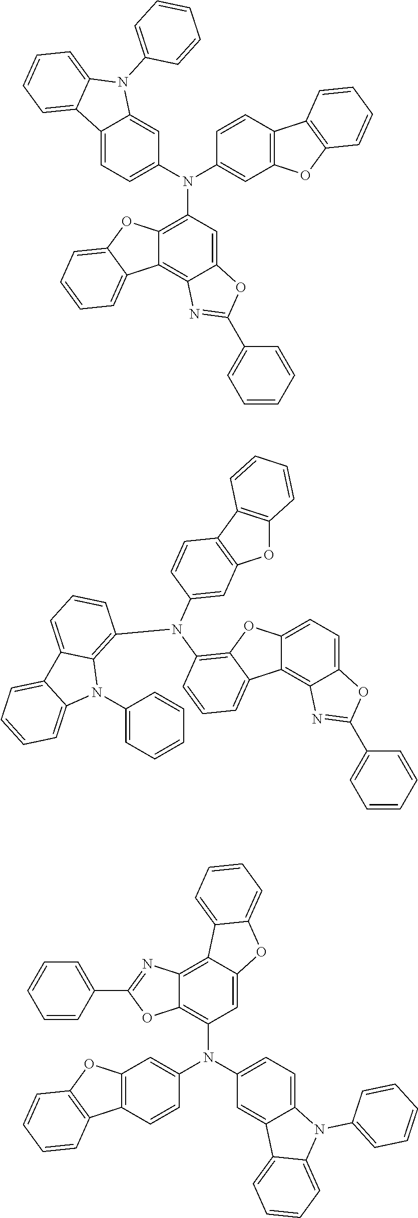 [SMILES: c1ccc(-c2nc3c(cc(N(c4ccc5c(c4)oc4ccccc45)c4ccc5c6ccccc6n(-c6ccccc6)c5c4)c4oc5ccccc5c43)o2)cc1 ; c1ccc(-c2nc3c(ccc4oc5c(N(c6ccc7c(c6)oc6ccccc67)c6cccc7c8ccccc8n(-c8ccccc8)c67)cccc5c43)o2)cc1 ; c1ccc(-c2nc3c(o2)c(N(c2ccc4c(c2)oc2ccccc24)c2ccc4c(c2)c2ccccc2n4-c2ccccc2)cc2oc4ccccc4c23)cc1]

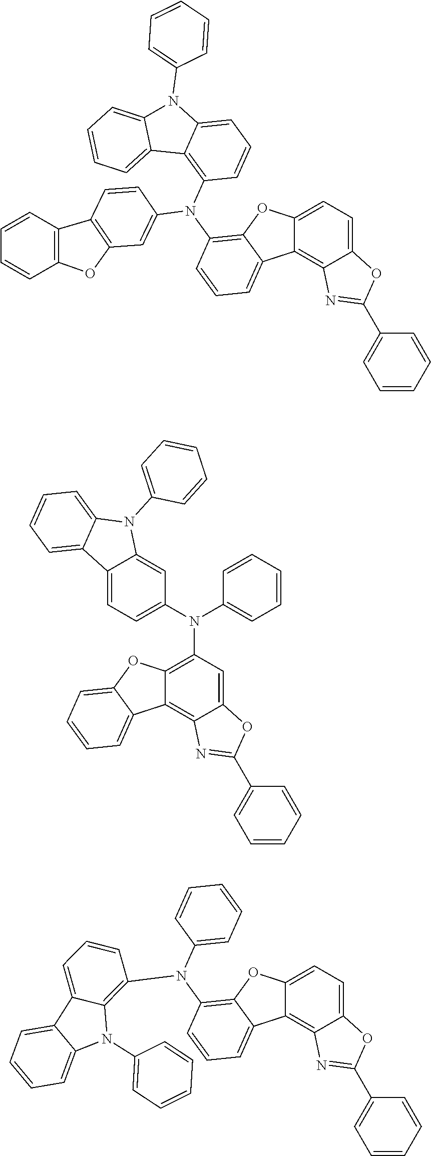 [SMILES: c1ccc(-c2nc3c(ccc4oc5c(N(c6ccc7c(c6)oc6ccccc67)c6cccc7c6c6ccccc6n7-c6ccccc6)cccc5c43)o2)cc1 ; c1ccc(-c2nc3c(cc(N(c4ccccc4)c4ccc5c6ccccc6n(-c6ccccc6)c5c4)c4oc5ccccc5c43)o2)cc1 ; c1ccc(-c2nc3c(ccc4oc5c(N(c6ccccc6)c6cccc7c8ccccc8n(-c8ccccc8)c67)cccc5c43)o2)cc1]

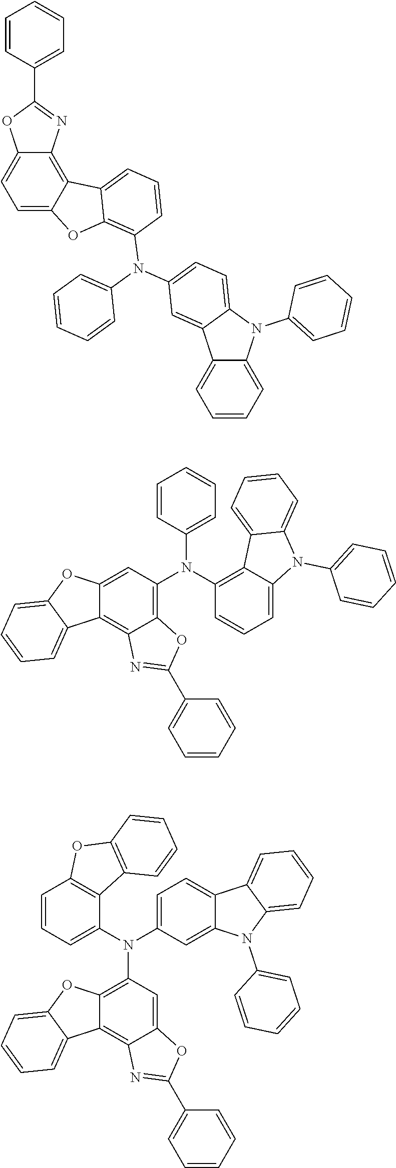 [SMILES: c1ccc(-c2nc3c(ccc4oc5c(N(c6ccccc6)c6ccc7c(c6)c6ccccc6n7-c6ccccc6)cccc5c43)o2)cc1 ; c1ccc(-c2nc3c(o2)c(N(c2ccccc2)c2cccc4c2c2ccccc2n4-c2ccccc2)cc2oc4ccccc4c23)cc1 ; c1ccc(-c2nc3c(cc(N(c4ccc5c6ccccc6n(-c6ccccc6)c5c4)c4cccc5oc6ccccc6c45)c4oc5ccccc5c43)o2)cc1]

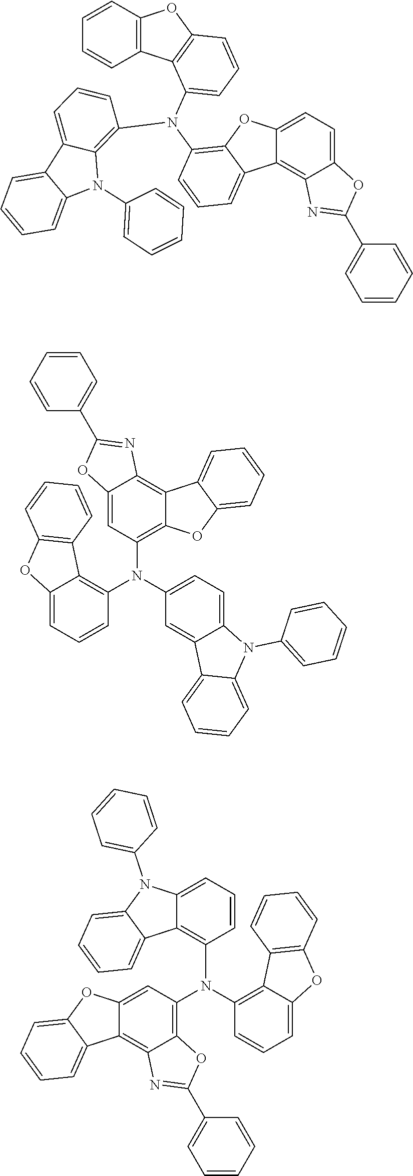 [SMILES: c1ccc(-c2nc3c(ccc4oc5c(N(c6cccc7oc8ccccc8c67)c6cccc7c8ccccc8n(-c8ccccc8)c67)cccc5c43)o2)cc1 ; c1ccc(-c2nc3c(cc(N(c4ccc5c(c4)c4ccccc4n5-c4ccccc4)c4cccc5oc6ccccc6c45)c4oc5ccccc5c43)o2)cc1 ; c1ccc(-c2nc3c(o2)c(N(c2cccc4oc5ccccc5c24)c2cccc4c2c2ccccc2n4-c2ccccc2)cc2oc4ccccc4c23)cc1]

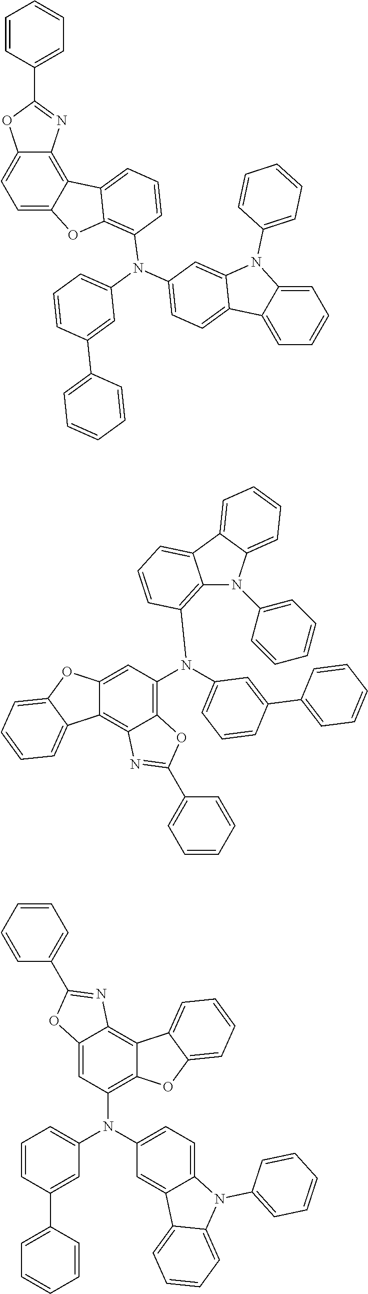 [SMILES: c1ccc(-c2cccc(N(c3ccc4c5ccccc5n(-c5ccccc5)c4c3)c3cccc4c3oc3ccc5oc(-c6ccccc6)nc5c34)c2)cc1 ; c1ccc(-c2cccc(N(c3cc4oc5ccccc5c4c4nc(-c5ccccc5)oc34)c3cccc4c5ccccc5n(-c5ccccc5)c34)c2)cc1 ; c1ccc(-c2cccc(N(c3ccc4c(c3)c3ccccc3n4-c3ccccc3)c3cc4oc(-c5ccccc5)nc4c4c3oc3ccccc34)c2)cc1]

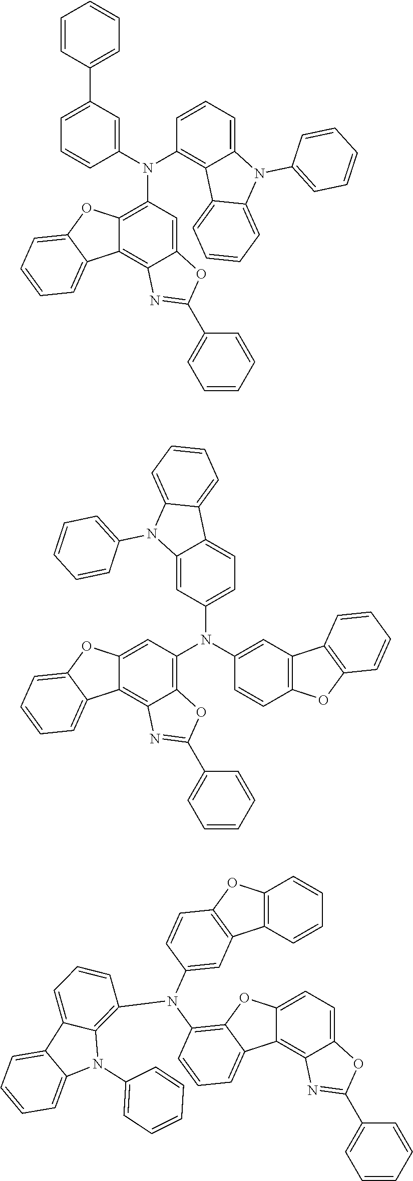

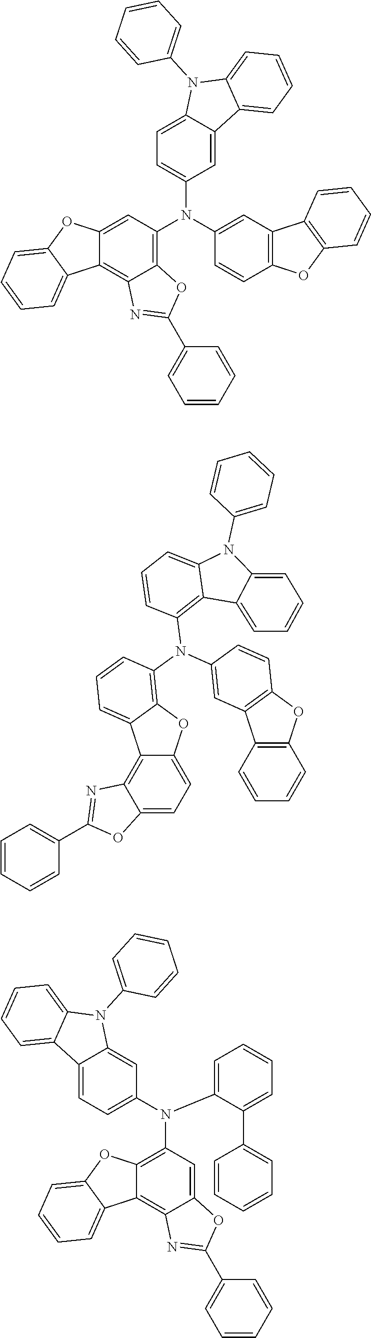 [SMILES: c1ccc(-c2nc3c(o2)c(N(c2ccc4oc5ccccc5c4c2)c2ccc4c(c2)c2ccccc2n4-c2ccccc2)cc2oc4ccccc4c23)cc1 ; c1ccc(-c2nc3c(ccc4oc5c(N(c6ccc7oc8ccccc8c7c6)c6cccc7c6c6ccccc6n7-c6ccccc6)cccc5c43)o2)cc1 ; c1ccc(-c2nc3c(cc(N(c4ccc5c6ccccc6n(-c6ccccc6)c5c4)c4ccccc4-c4ccccc4)c4oc5ccccc5c43)o2)cc1]

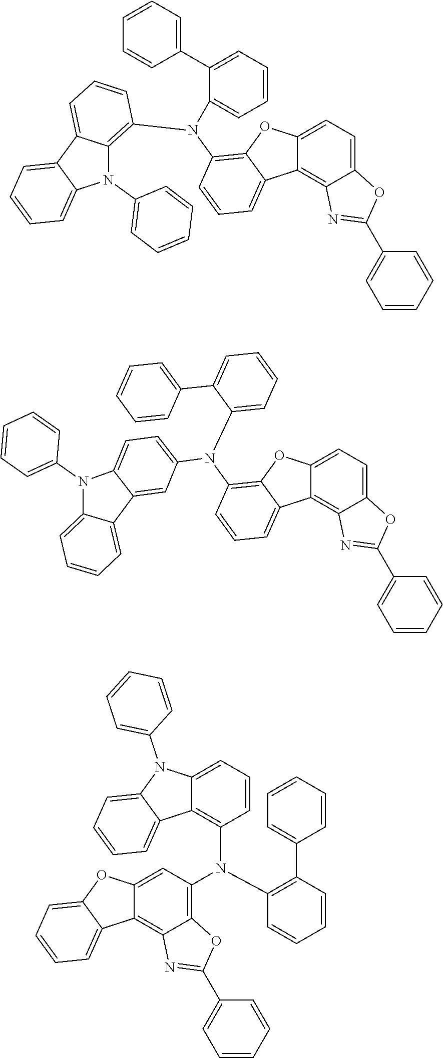 [SMILES: c1ccc(-c2nc3c(ccc4oc5c(N(c6ccccc6-c6ccccc6)c6cccc7c8ccccc8n(-c8ccccc8)c67)cccc5c43)o2)cc1 ; c1ccc(-c2nc3c(ccc4oc5c(N(c6ccc7c(c6)c6ccccc6n7-c6ccccc6)c6ccccc6-c6ccccc6)cccc5c43)o2)cc1 ; c1ccc(-c2nc3c(o2)c(N(c2ccccc2-c2ccccc2)c2cccc4c2c2ccccc2n4-c2ccccc2)cc2oc4ccccc4c23)cc1]

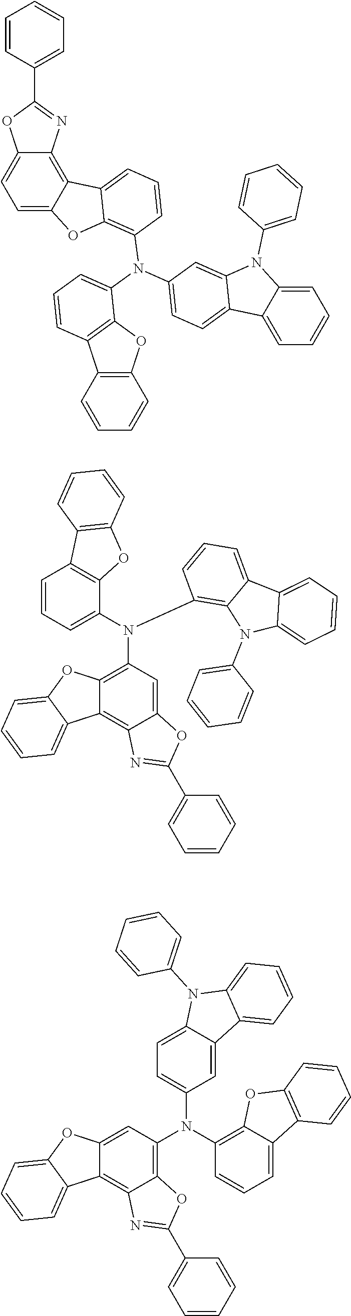

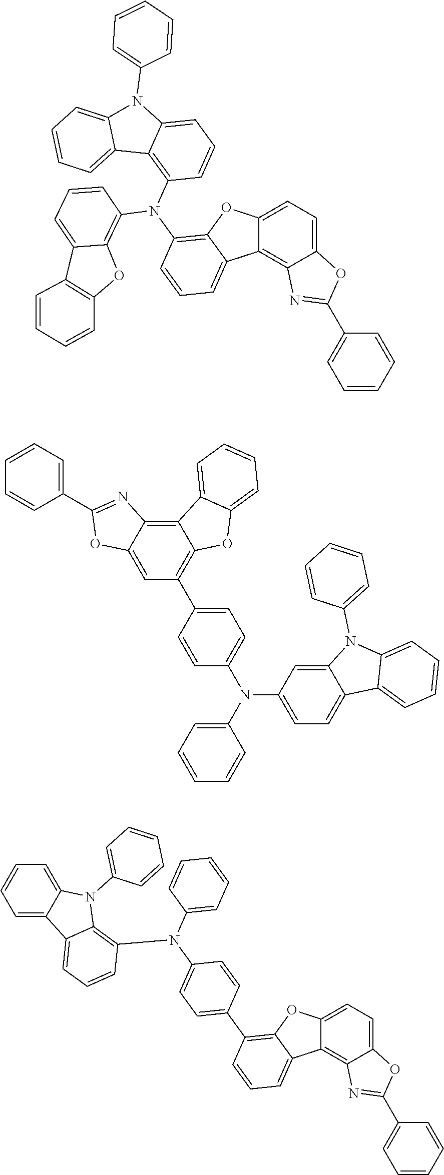 [SMILES: c1ccc(-c2nc3c(ccc4oc5c(N(c6cccc7c6oc6ccccc67)c6cccc7c6c6ccccc6n7-c6ccccc6)cccc5c43)o2)cc1 ; c1ccc(-c2nc3c(cc(-c4ccc(N(c5ccccc5)c5ccc6c7ccccc7n(-c7ccccc7)c6c5)cc4)c4oc5ccccc5c43)o2)cc1 ; c1ccc(-c2nc3c(ccc4oc5c(-c6ccc(N(c7ccccc7)c7cccc8c9ccccc9n(-c9ccccc9)c78)cc6)cccc5c43)o2)cc1]

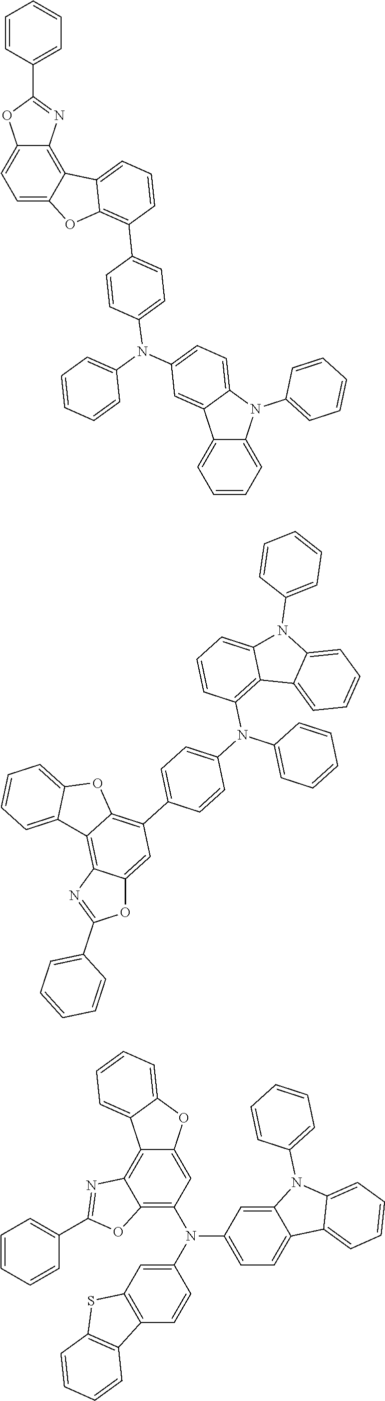 [SMILES: c1ccc(-c2nc3c(ccc4oc5c(-c6ccc(N(c7ccccc7)c7ccc8c(c7)c7ccccc7n8-c7ccccc7)cc6)cccc5c43)o2)cc1 ; c1ccc(-c2nc3c(cc(-c4ccc(N(c5ccccc5)c5cccc6c5c5ccccc5n6-c5ccccc5)cc4)c4oc5ccccc5c43)o2)cc1 ; c1ccc(-c2nc3c(o2)c(N(c2ccc4c(c2)sc2ccccc24)c2ccc4c5ccccc5n(-c5ccccc5)c4c2)cc2oc4ccccc4c23)cc1]

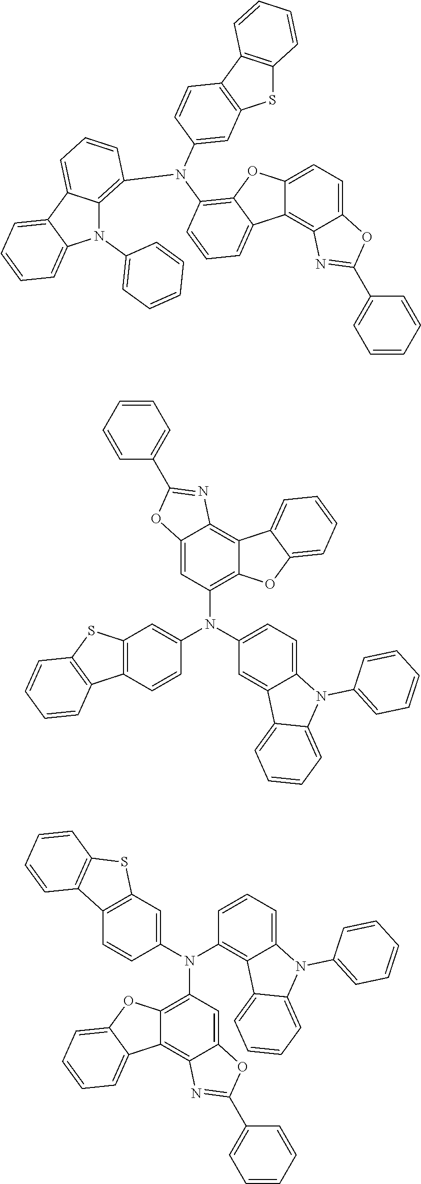 [SMILES: c1ccc(-c2nc3c(ccc4oc5c(N(c6ccc7c(c6)sc6ccccc67)c6cccc7c8ccccc8n(-c8ccccc8)c67)cccc5c43)o2)cc1 ; c1ccc(-c2nc3c(cc(N(c4ccc5c(c4)sc4ccccc45)c4ccc5c(c4)c4ccccc4n5-c4ccccc4)c4oc5ccccc5c43)o2)cc1 ; c1ccc(-c2nc3c(cc(N(c4ccc5c(c4)sc4ccccc45)c4cccc5c4c4ccccc4n5-c4ccccc4)c4oc5ccccc5c43)o2)cc1]

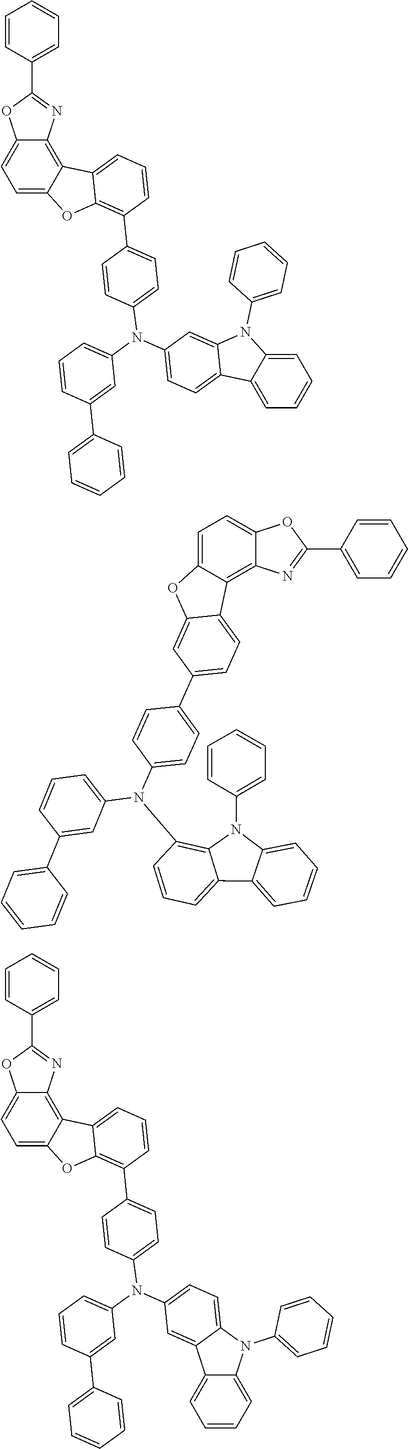

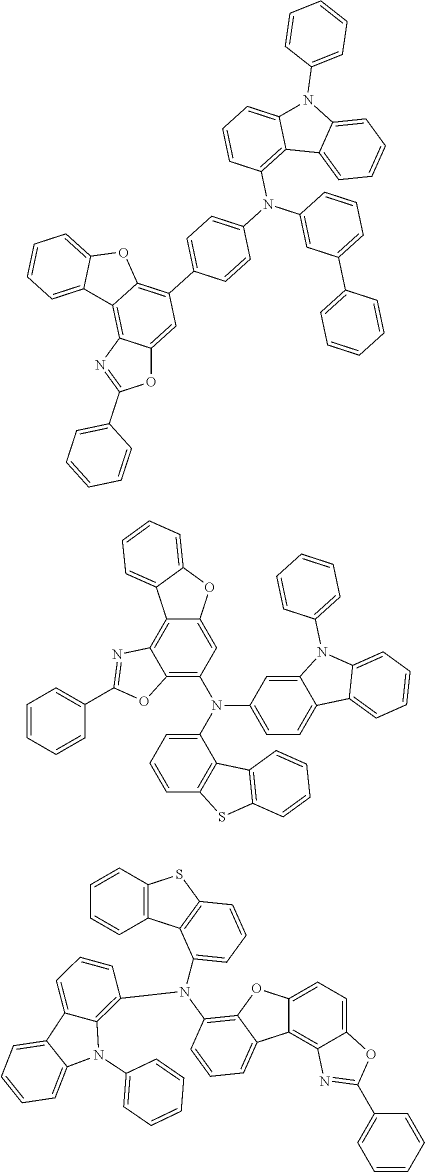 [SMILES: c1ccc(-c2cccc(N(c3ccc(-c4cc5oc(-c6ccccc6)nc5c5c4oc4ccccc45)cc3)c3cccc4c3c3ccccc3n4-c3ccccc3)c2)cc1 ; c1ccc(-c2nc3c(o2)c(N(c2ccc4c5ccccc5n(-c5ccccc5)c4c2)c2cccc4sc5ccccc5c24)cc2oc4ccccc4c23)cc1 ; c1ccc(-c2nc3c(ccc4oc5c(N(c6cccc7sc8ccccc8c67)c6cccc7c8ccccc8n(-c8ccccc8)c67)cccc5c43)o2)cc1]

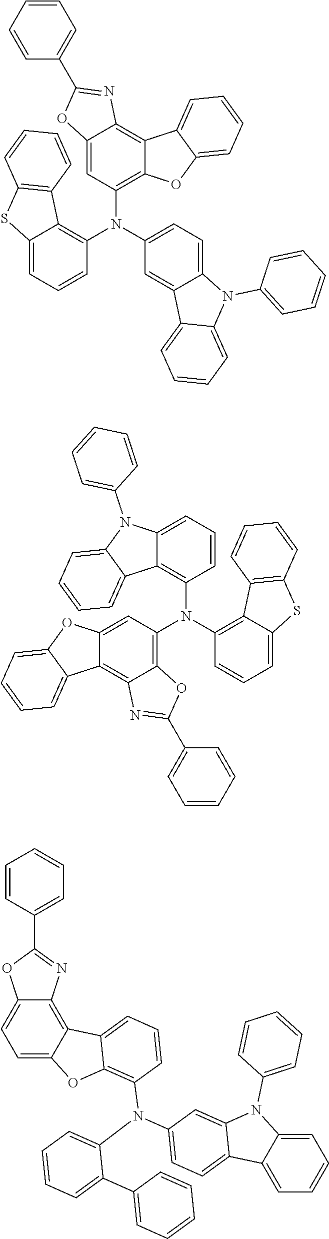 [SMILES: c1ccc(-c2nc3c(cc(N(c4ccc5c(c4)c4ccccc4n5-c4ccccc4)c4cccc5sc6ccccc6c45)c4oc5ccccc5c43)o2)cc1 ; c1ccc(-c2nc3c(o2)c(N(c2cccc4sc5ccccc5c24)c2cccc4c2c2ccccc2n4-c2ccccc2)cc2oc4ccccc4c23)cc1 ; c1ccc(-c2nc3c(ccc4oc5c(N(c6ccc7c8ccccc8n(-c8ccccc8)c7c6)c6ccccc6-c6ccccc6)cccc5c43)o2)cc1]

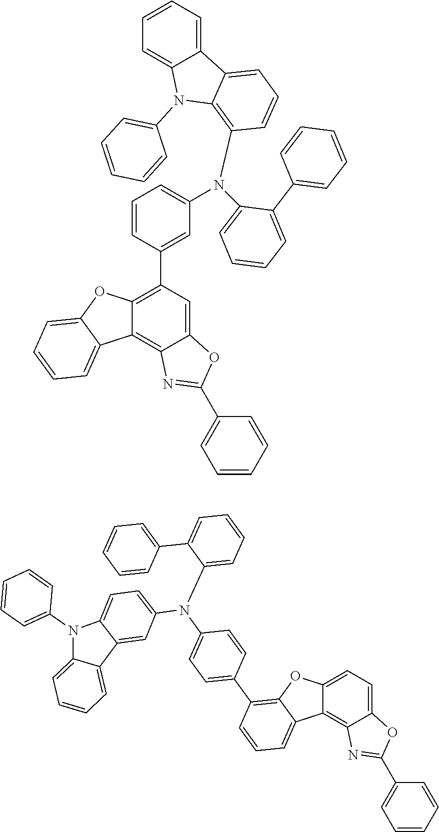

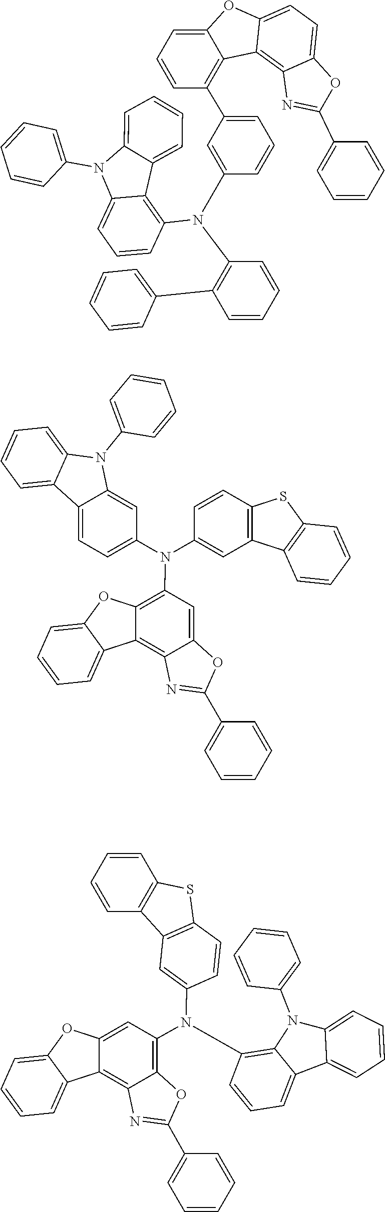 [SMILES: c1ccc(-c2nc3c(ccc4oc5cccc(-c6cccc(N(c7ccccc7-c7ccccc7)c7cccc8c7c7ccccc7n8-c7ccccc7)c6)c5c43)o2)cc1 ; c1ccc(-c2nc3c(cc(N(c4ccc5sc6ccccc6c5c4)c4ccc5c6ccccc6n(-c6ccccc6)c5c4)c4oc5ccccc5c43)o2)cc1 ; c1ccc(-c2nc3c(o2)c(N(c2ccc4sc5ccccc5c4c2)c2cccc4c5ccccc5n(-c5ccccc5)c24)cc2oc4ccccc4c23)cc1]

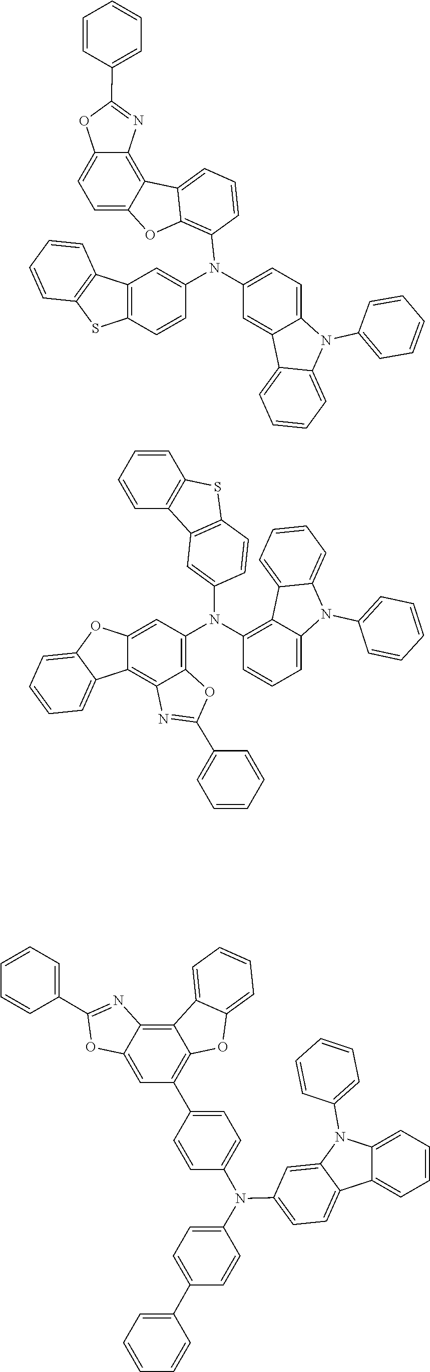 [SMILES: c1ccc(-c2nc3c(ccc4oc5c(N(c6ccc7sc8ccccc8c7c6)c6ccc7c(c6)c6ccccc6n7-c6ccccc6)cccc5c43)o2)cc1 ; c1ccc(-c2nc3c(o2)c(N(c2ccc4sc5ccccc5c4c2)c2cccc4c2c2ccccc2n4-c2ccccc2)cc2oc4ccccc4c23)cc1 ; c1ccc(-c2ccc(N(c3ccc(-c4cc5oc(-c6ccccc6)nc5c5c4oc4ccccc45)cc3)c3ccc4c5ccccc5n(-c5ccccc5)c4c3)cc2)cc1]

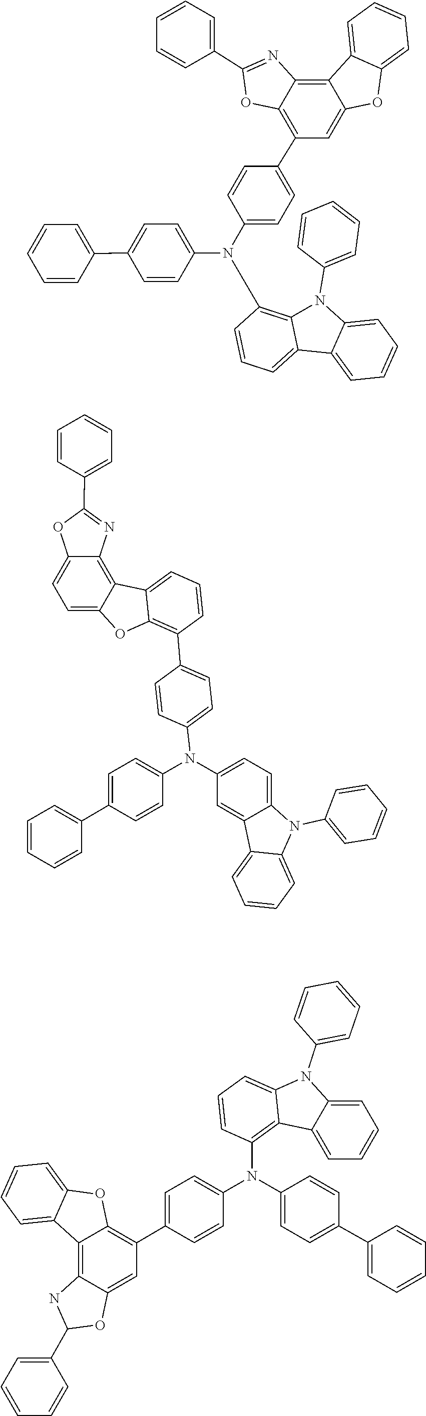

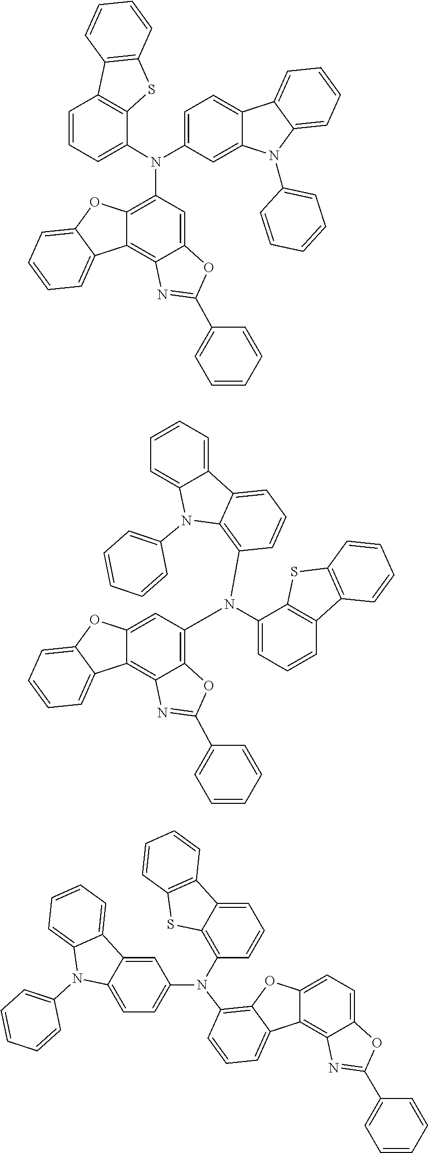

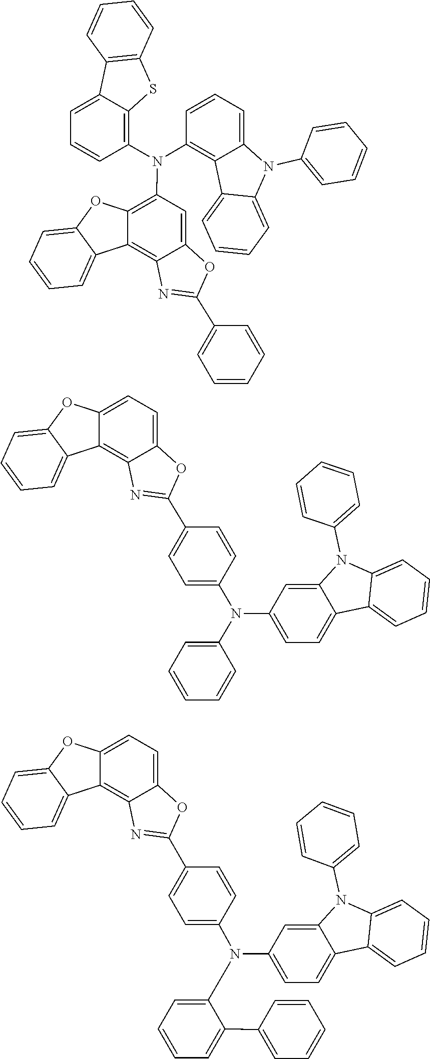 [SMILES: c1ccc(-c2nc3c(cc(N(c4cccc5c4sc4ccccc45)c4cccc5c4c4ccccc4n5-c4ccccc4)c4oc5ccccc5c43)o2)cc1 ; c1ccc(N(c2ccc(-c3nc4c(ccc5oc6ccccc6c54)o3)cc2)c2ccc3c4ccccc4n(-c4ccccc4)c3c2)cc1 ; c1ccc(-c2ccccc2N(c2ccc(-c3nc4c(ccc5oc6ccccc6c54)o3)cc2)c2ccc3c4ccccc4n(-c4ccccc4)c3c2)cc1]

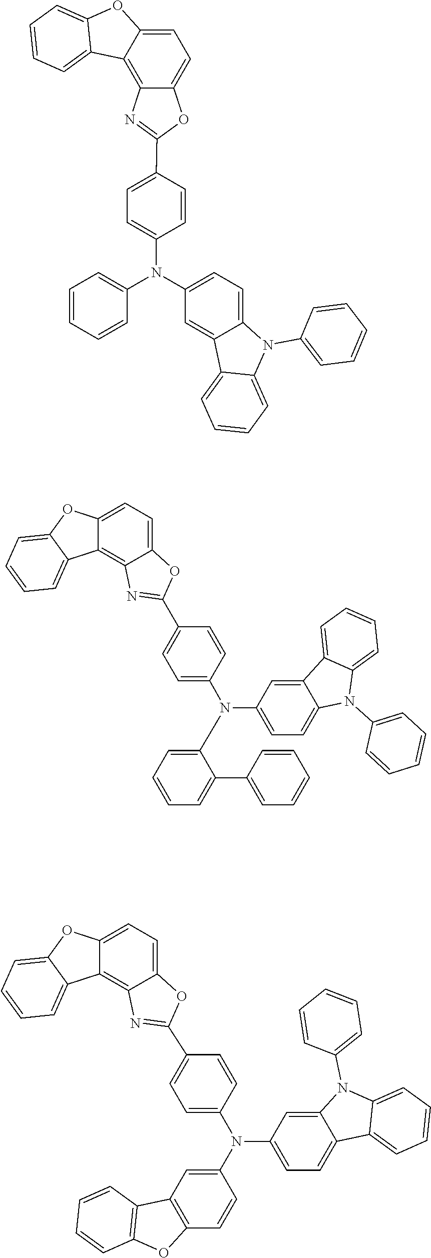 [SMILES: c1ccc(N(c2ccc(-c3nc4c(ccc5oc6ccccc6c54)o3)cc2)c2ccc3c(c2)c2ccccc2n3-c2ccccc2)cc1 ; c1ccc(-c2ccccc2N(c2ccc(-c3nc4c(ccc5oc6ccccc6c54)o3)cc2)c2ccc3c(c2)c2ccccc2n3-c2ccccc2)cc1 ; c1ccc(-n2c3ccccc3c3ccc(N(c4ccc(-c5nc6c(ccc7oc8ccccc8c76)o5)cc4)c4ccc5oc6ccccc6c5c4)cc32)cc1]

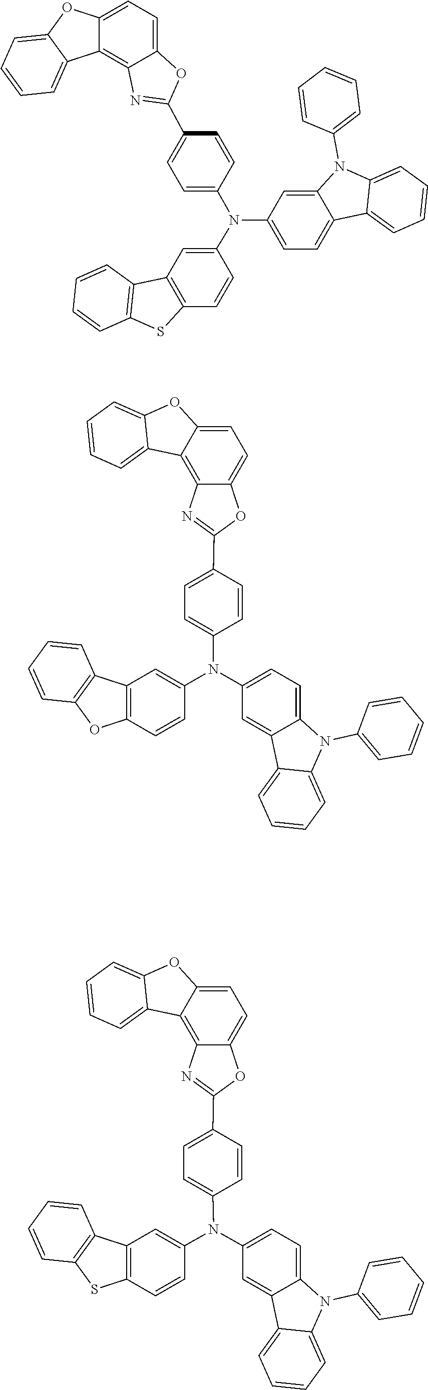

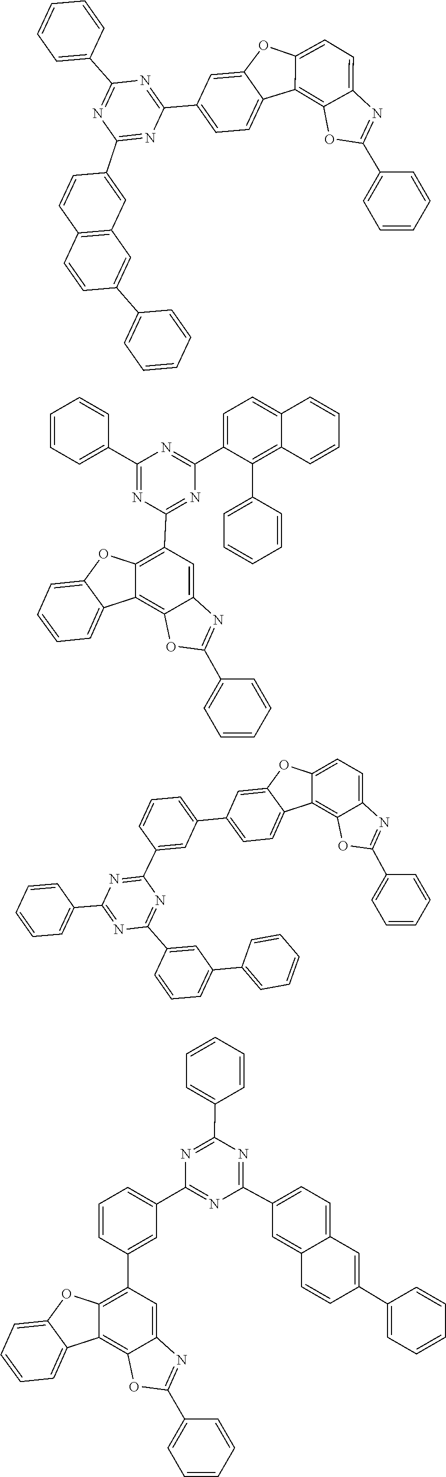

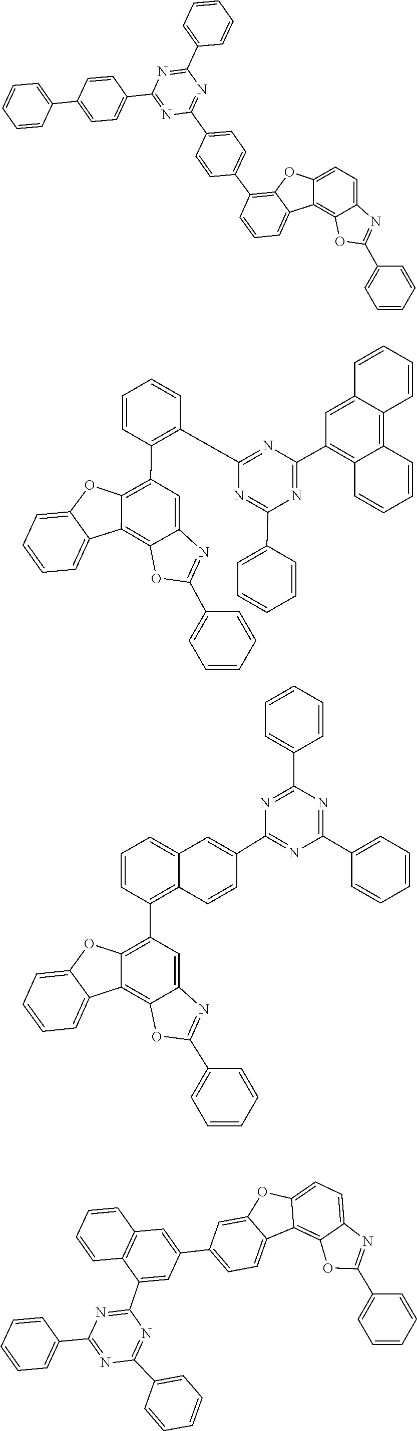

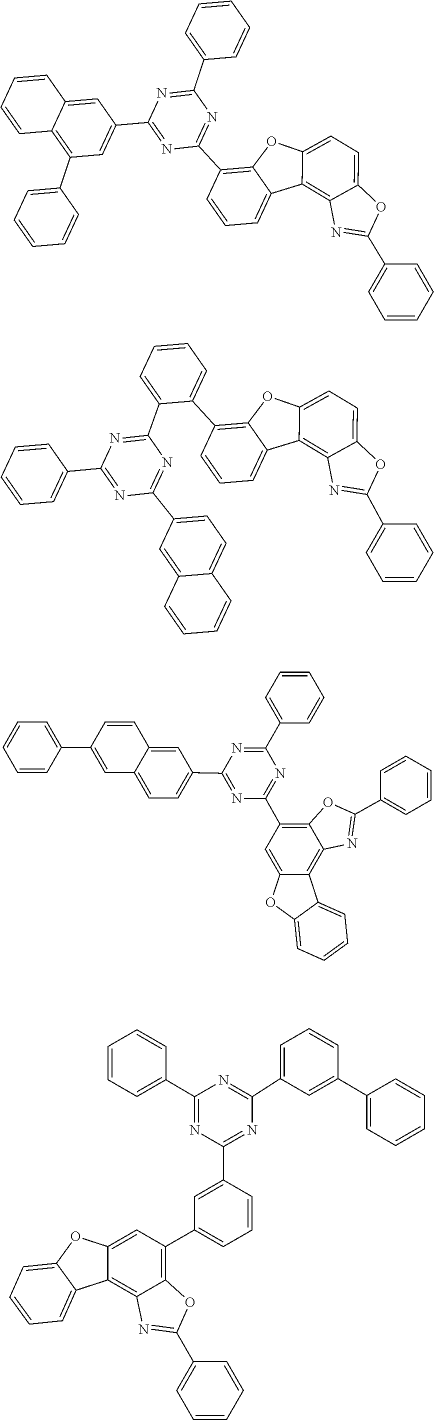 [SMILES: c1ccc(-c2nc(-c3cc(-c4ccccc4)c4ccccc4c3)nc(-c3cccc4c3oc3ccc5oc(-c6ccccc6)nc5c34)n2)cc1 ; c1ccc(-c2nc(-c3ccc4ccccc4c3)nc(-c3ccccc3-c3cccc4c3oc3ccc5oc(-c6ccccc6)nc5c34)n2)cc1 ; c1ccc(-c2ccc3cc(-c4nc(-c5ccccc5)nc(-c5cc6oc7ccccc7c6c6nc(-c7ccccc7)oc56)n4)ccc3c2)cc1 ; c1ccc(-c2cccc(-c3nc(-c4ccccc4)nc(-c4cccc(-c5cc6oc7ccccc7c6c6nc(-c7ccccc7)oc56)c4)n3)c2)cc1]

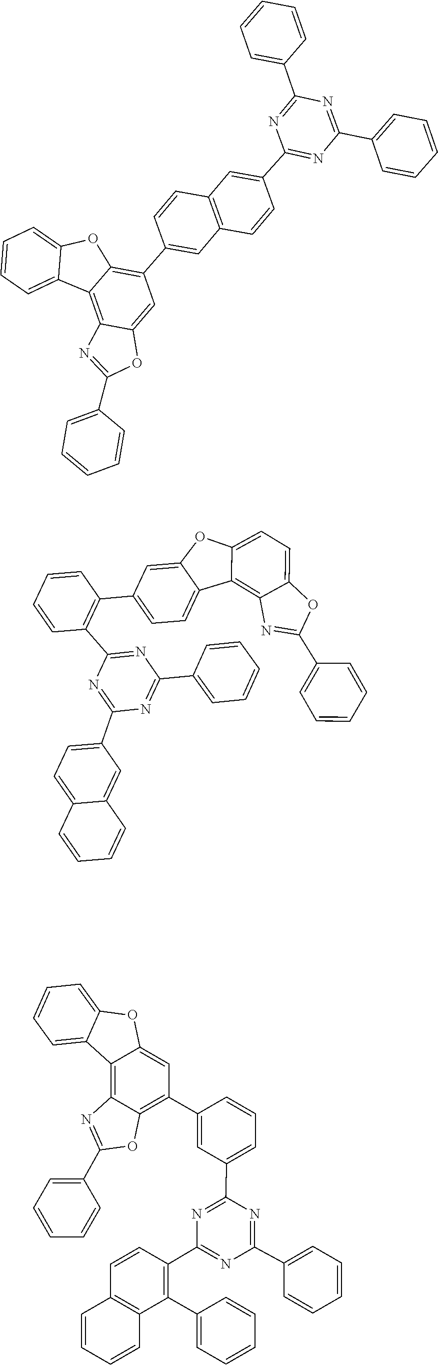 [SMILES: c1ccc(-c2nc(-c3ccccc3)nc(-c3ccc4cc(-c5cc6oc(-c7ccccc7)nc6c6c5oc5ccccc56)ccc4c3)n2)cc1 ; c1ccc(-c2nc(-c3ccc4ccccc4c3)nc(-c3ccccc3-c3ccc4c(c3)oc3ccc5oc(-c6ccccc6)nc5c34)n2)cc1 ; c1ccc(-c2nc(-c3cccc(-c4cc5oc6ccccc6c5c5nc(-c6ccccc6)oc45)c3)nc(-c3ccc4ccccc4c3-c3ccccc3)n2)cc1]

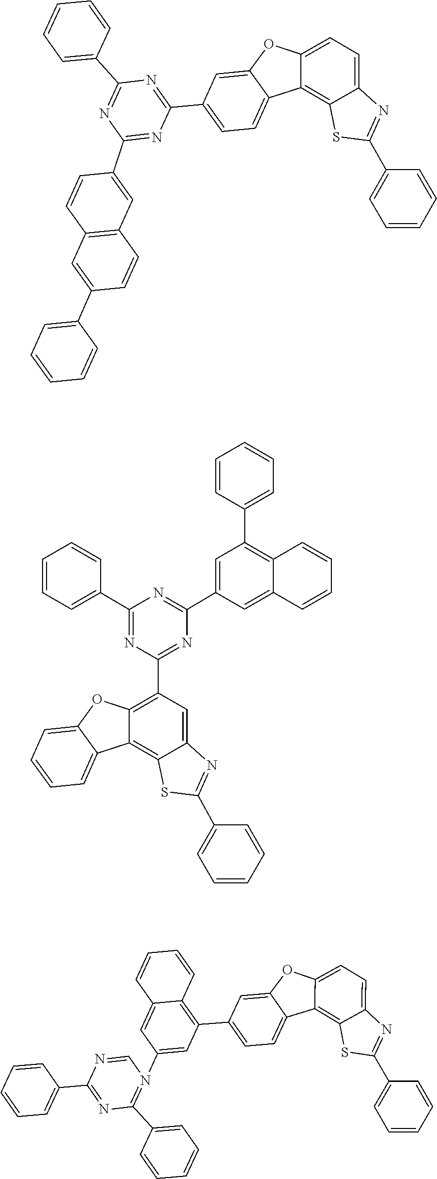

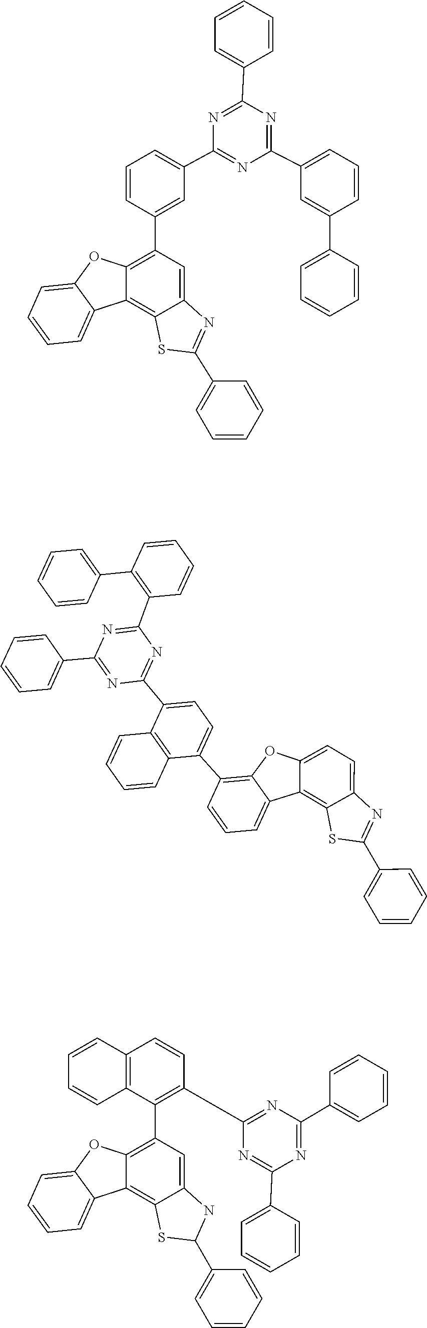

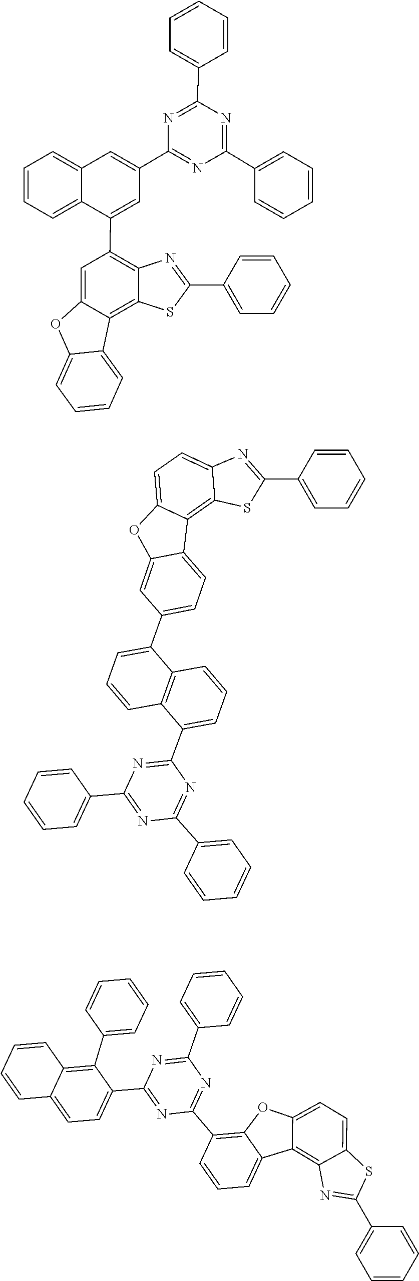 [SMILES: c1ccc(-c2nc(-c3ccccc3)nc(-c3cc(-c4cc5oc6ccccc6c5c5sc(-c6ccccc6)nc45)c4ccccc4c3)n2)cc1 ; c1ccc(-c2nc(-c3ccccc3)nc(-c3cccc4c(-c5ccc6c(c5)oc5ccc7nc(-c8ccccc8)sc7c56)cccc34)n2)cc1 ; c1ccc(-c2nc(-c3ccc4ccccc4c3-c3ccccc3)nc(-c3cccc4c3oc3ccc5sc(-c6ccccc6)nc5c34)n2)cc1]

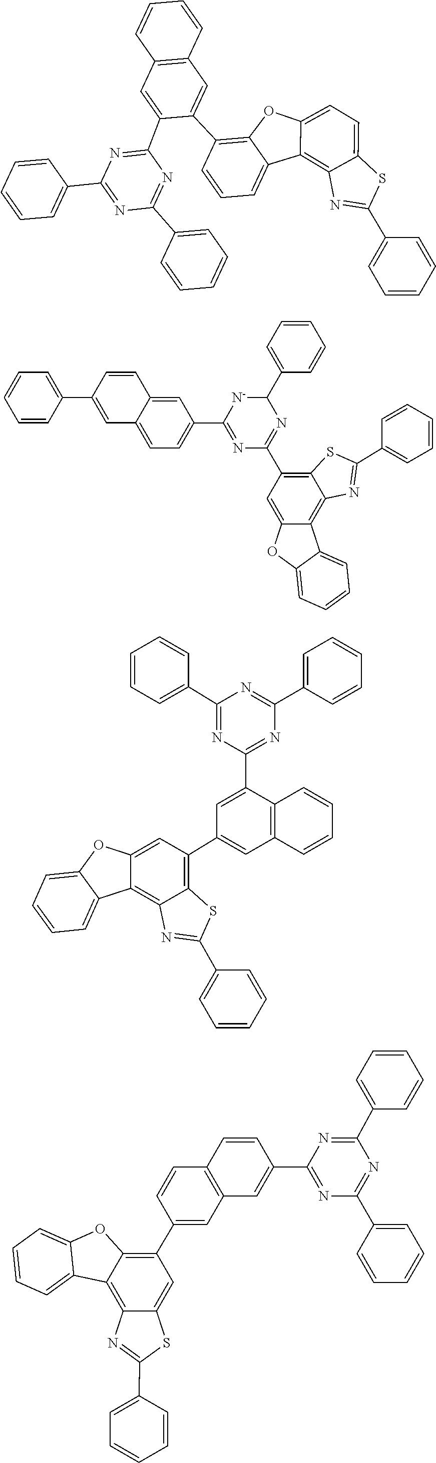 [SMILES: c1ccc(-c2nc(-c3ccccc3)nc(-c3cc4ccccc4cc3-c3cccc4c3oc3ccc5sc(-c6ccccc6)nc5c34)n2)cc1 ; c1ccc(-c2ccc3cc(C4=NC(c5cc6oc7ccccc7c6c6nc(-c7ccccc7)sc56)=NC(c5ccccc5)[N-]4)ccc3c2)cc1 ; c1ccc(-c2nc(-c3ccccc3)nc(-c3cc(-c4cc5oc6ccccc6c5c5nc(-c6ccccc6)sc45)cc4ccccc34)n2)cc1 ; c1ccc(-c2nc(-c3ccccc3)nc(-c3ccc4ccc(-c5cc6sc(-c7ccccc7)nc6c6c5oc5ccccc56)cc4c3)n2)cc1]

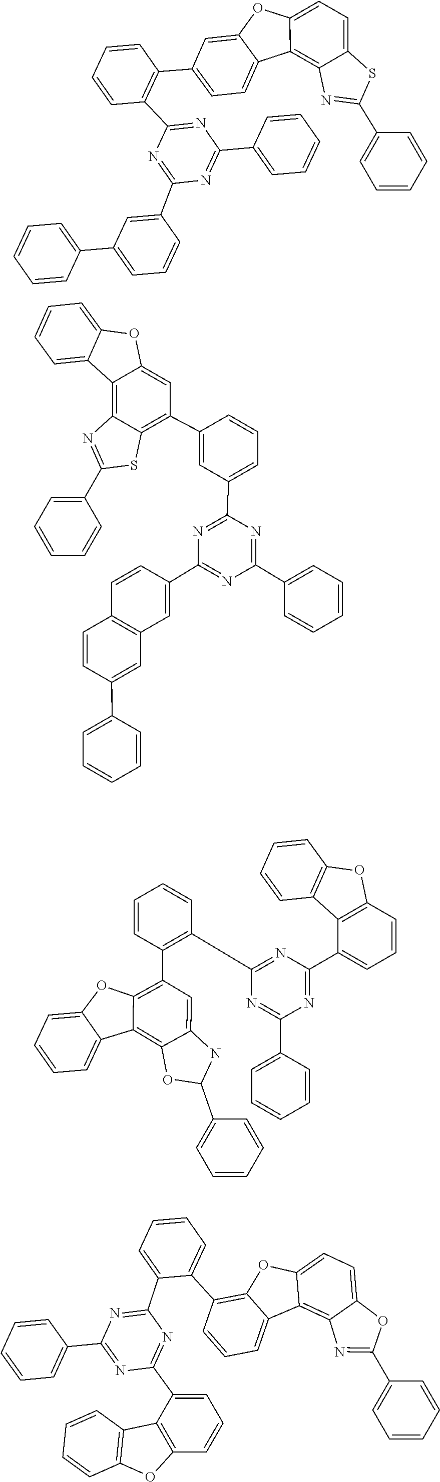

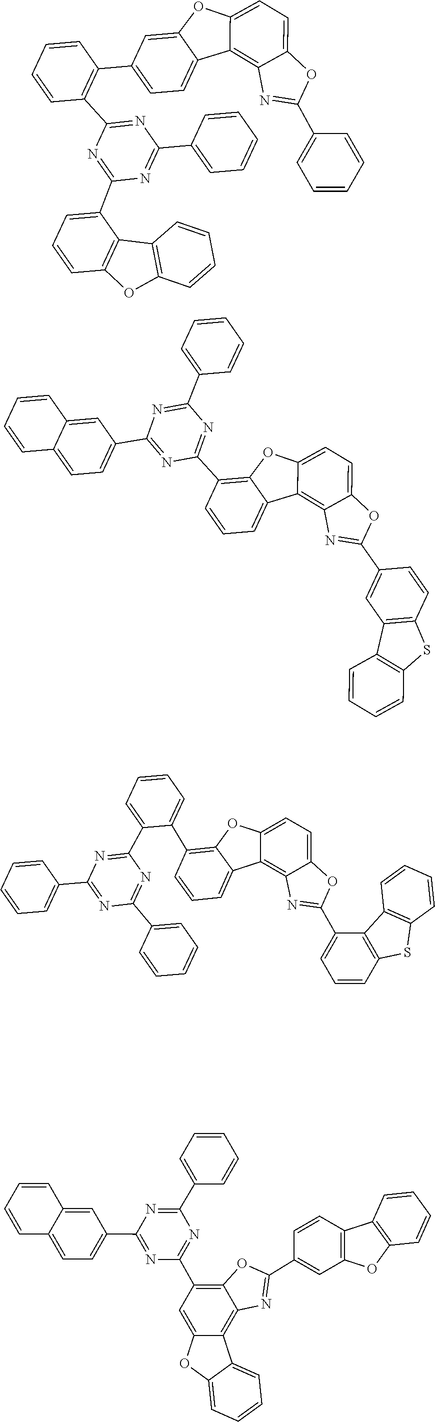

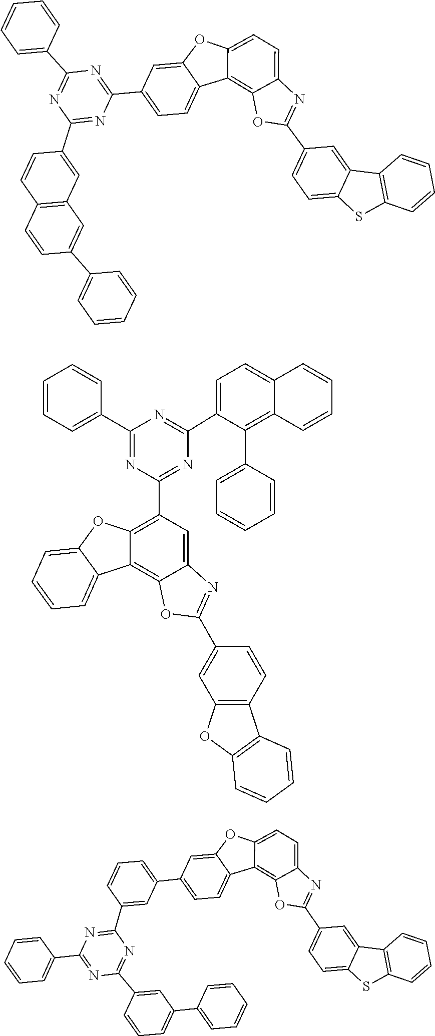

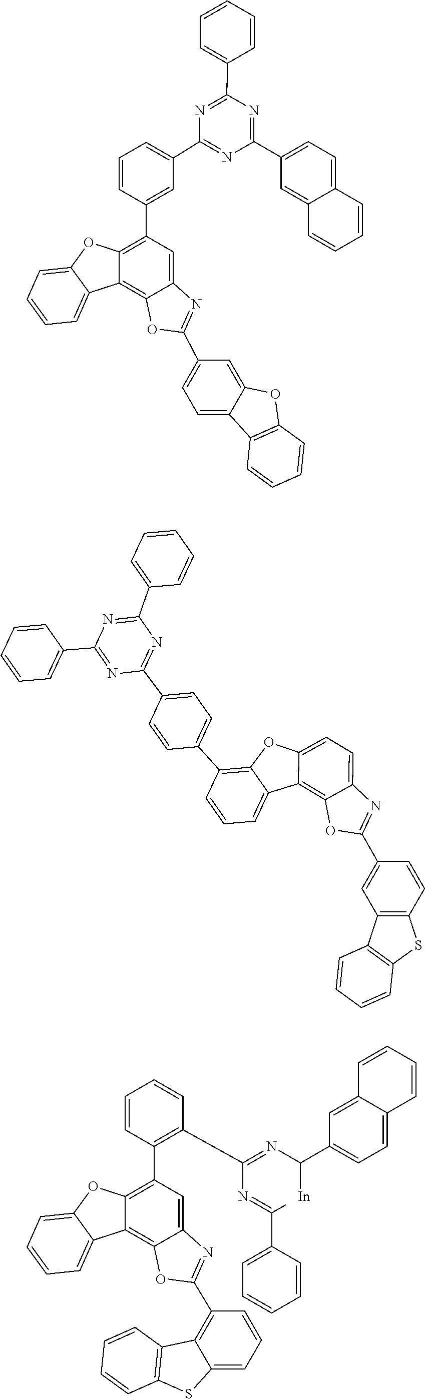

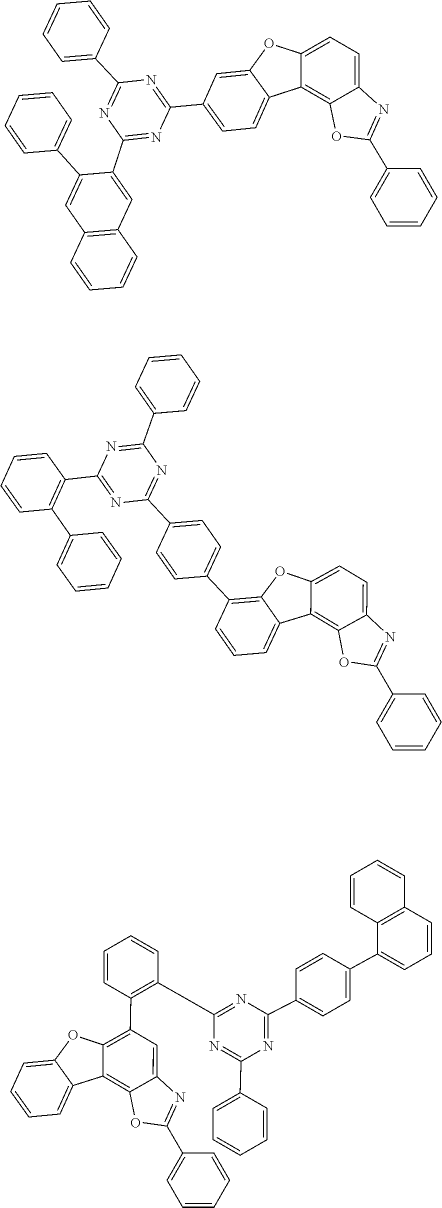 [SMILES: c1ccc(-c2nc(-c3ccc4c(c3)oc3ccc5nc(-c6ccccc6)oc5c34)nc(-c3cc4ccccc4cc3-c3ccccc3)n2)cc1 ; c1ccc(-c2nc(-c3ccc(-c4cccc5c4oc4ccc6nc(-c7ccccc7)oc6c45)cc3)nc(-c3ccccc3-c3ccccc3)n2)cc1 ; c1ccc(-c2nc(-c3ccc(-c4cccc5ccccc45)cc3)nc(-c3ccccc3-c3cc4nc(-c5ccccc5)oc4c4c3oc3ccccc34)n2)cc1]

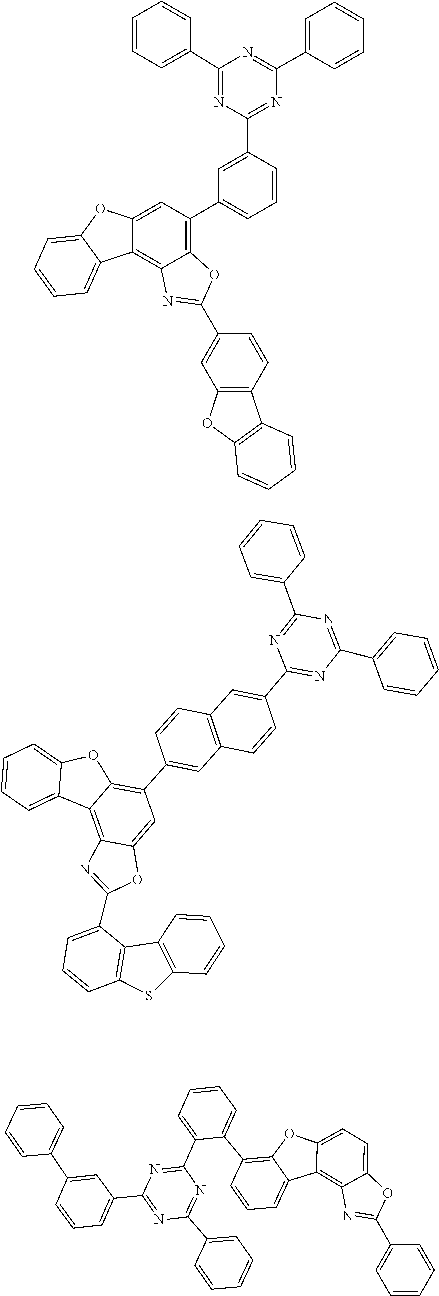

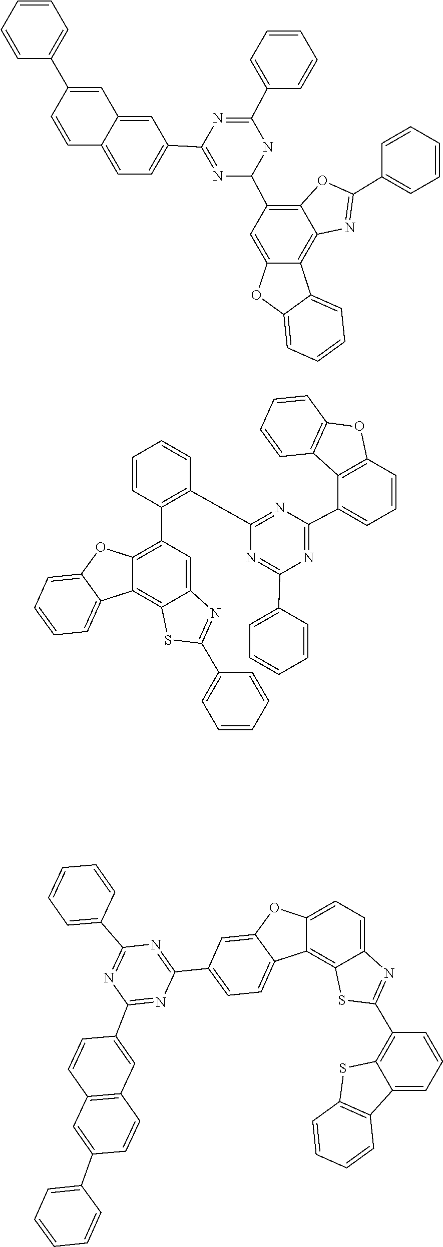

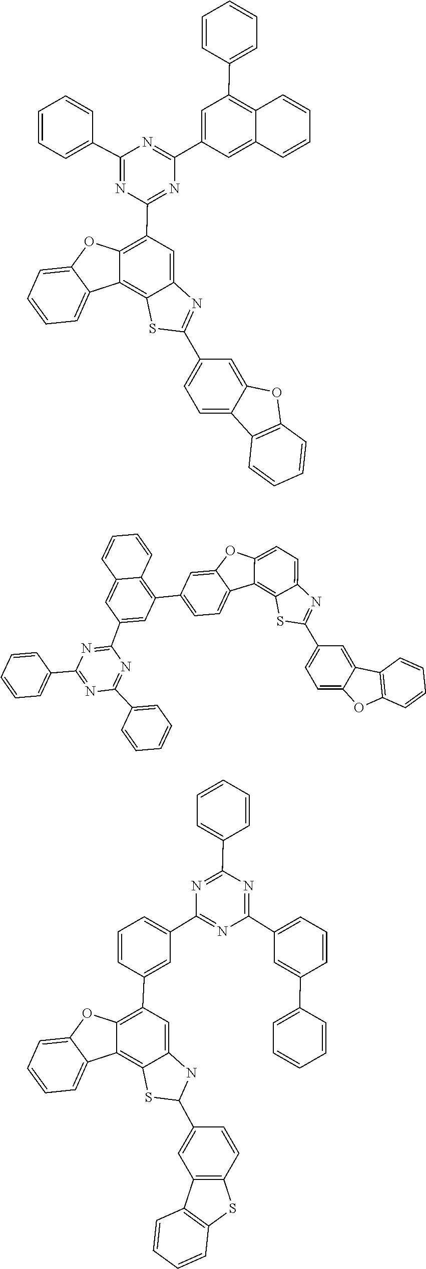

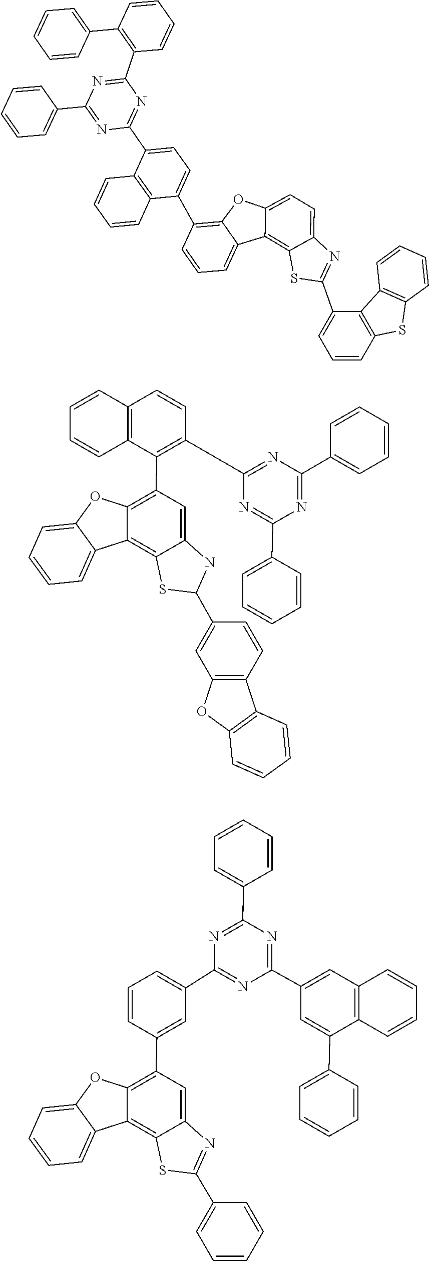

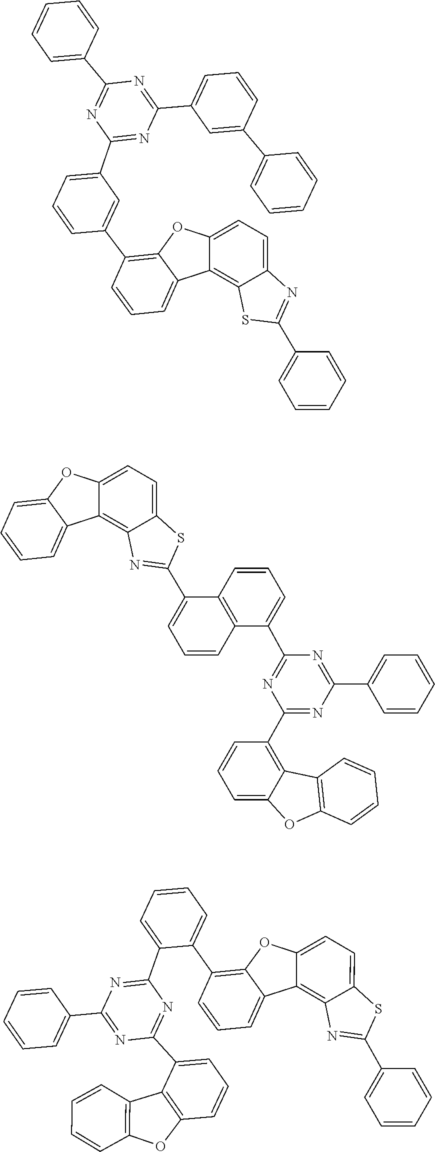 [SMILES: c1ccc(-c2cccc(-c3nc(-c4ccccc4)nc(-c4cccc(-c5cccc6c5oc5ccc7nc(-c8ccccc8)sc7c56)c4)n3)c2)cc1 ; c1ccc(-c2nc(-c3cccc4c(-c5nc6c(ccc7oc8ccccc8c76)s5)cccc34)nc(-c3cccc4oc5ccccc5c34)n2)cc1 ; c1ccc(-c2nc(-c3ccccc3-c3cccc4c3oc3ccc5sc(-c6ccccc6)nc5c34)nc(-c3cccc4oc5ccccc5c34)n2)cc1]

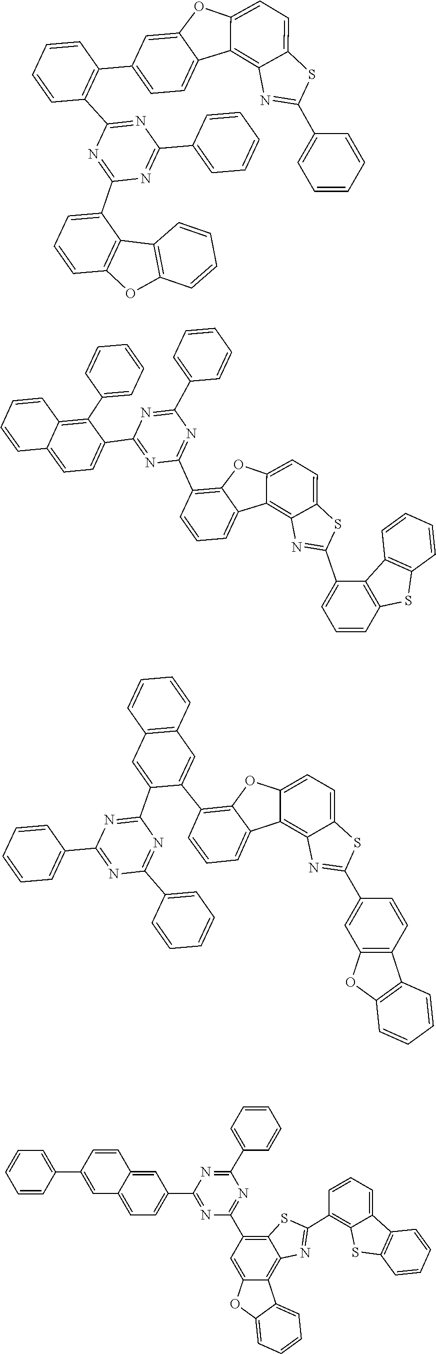 [SMILES: c1ccc(-c2nc(-c3ccccc3-c3ccc4c(c3)oc3ccc5sc(-c6ccccc6)nc5c34)nc(-c3cccc4oc5ccccc5c34)n2)cc1 ; c1ccc(-c2nc(-c3ccc4ccccc4c3-c3ccccc3)nc(-c3cccc4c3oc3ccc5sc(-c6cccc7sc8ccccc8c67)nc5c34)n2)cc1 ; c1ccc(-c2nc(-c3ccccc3)nc(-c3cc4ccccc4cc3-c3cccc4c3oc3ccc5sc(-c6ccc7c(c6)oc6ccccc67)nc5c34)n2)cc1 ; c1ccc(-c2ccc3cc(-c4nc(-c5ccccc5)nc(-c5cc6oc7ccccc7c6c6nc(-c7cccc8c7sc7ccccc78)sc56)n4)ccc3c2)cc1]

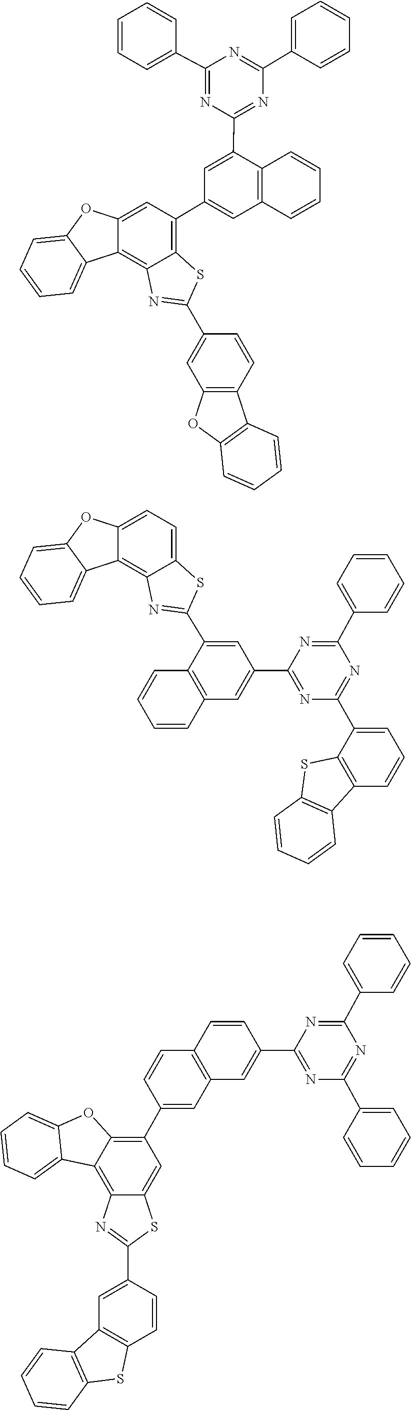 [SMILES: c1ccc(-c2nc(-c3ccccc3)nc(-c3cc(-c4cc5oc6ccccc6c5c5nc(-c6ccc7c(c6)oc6ccccc67)sc45)cc4ccccc34)n2)cc1 ; c1ccc(-c2nc(-c3cc(-c4nc5c(ccc6oc7ccccc7c65)s4)c4ccccc4c3)nc(-c3cccc4c3sc3ccccc34)n2)cc1 ; c1ccc(-c2nc(-c3ccccc3)nc(-c3ccc4ccc(-c5cc6sc(-c7ccc8sc9ccccc9c8c7)nc6c6c5oc5ccccc56)cc4c3)n2)cc1]

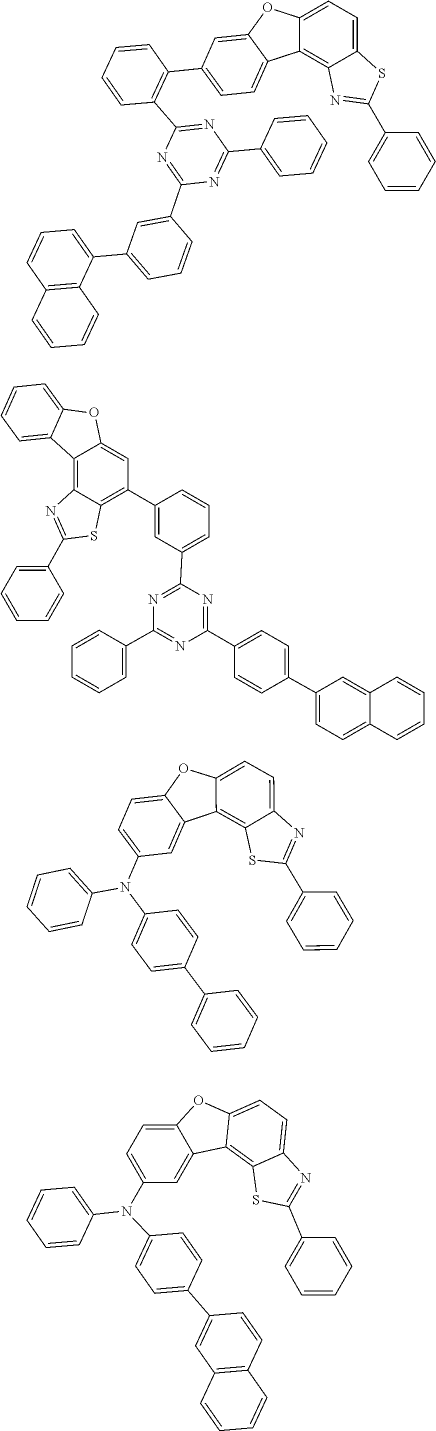

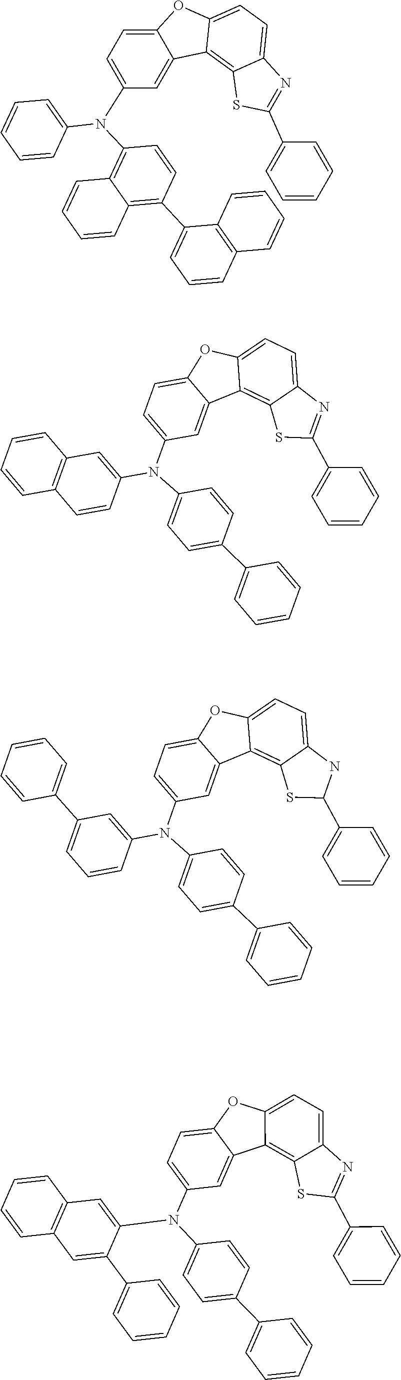

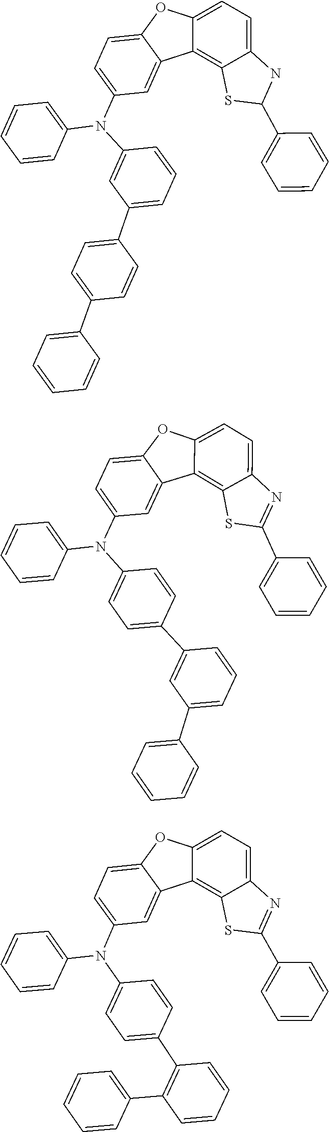

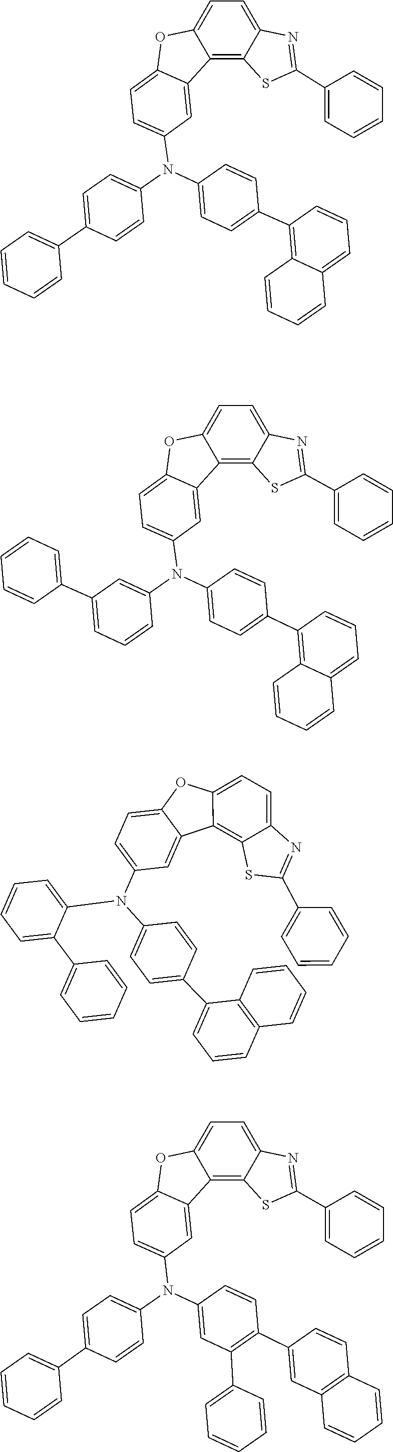 [SMILES: c1ccc(-c2ccc(N(c3ccc(-c4cccc5ccccc45)cc3)c3ccc4oc5ccc6nc(-c7ccccc7)sc6c5c4c3)cc2)cc1 ; c1ccc(-c2cccc(N(c3ccc(-c4cccc5ccccc45)cc3)c3ccc4oc5ccc6nc(-c7ccccc7)sc6c5c4c3)c2)cc1 ; c1ccc(-c2nc3ccc4oc5ccc(N(c6ccc(-c7cccc8ccccc78)cc6)c6ccccc6-c6ccccc6)cc5c4c3s2)cc1 ; c1ccc(-c2ccc(N(c3ccc(-c4ccc5ccccc5c4)c(-c4ccccc4)c3)c3ccc4oc5ccc6nc(-c7ccccc7)sc6c5c4c3)cc2)cc1]

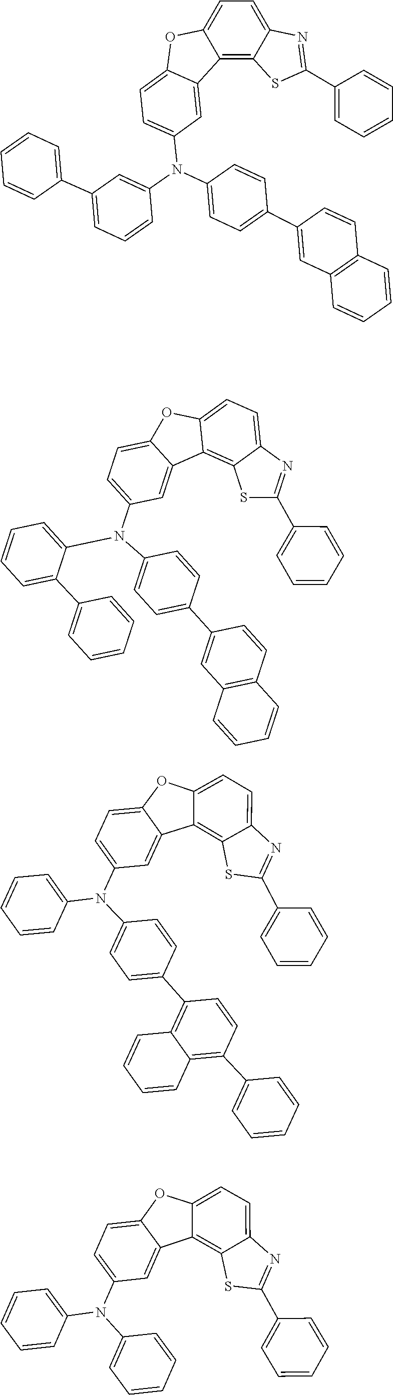

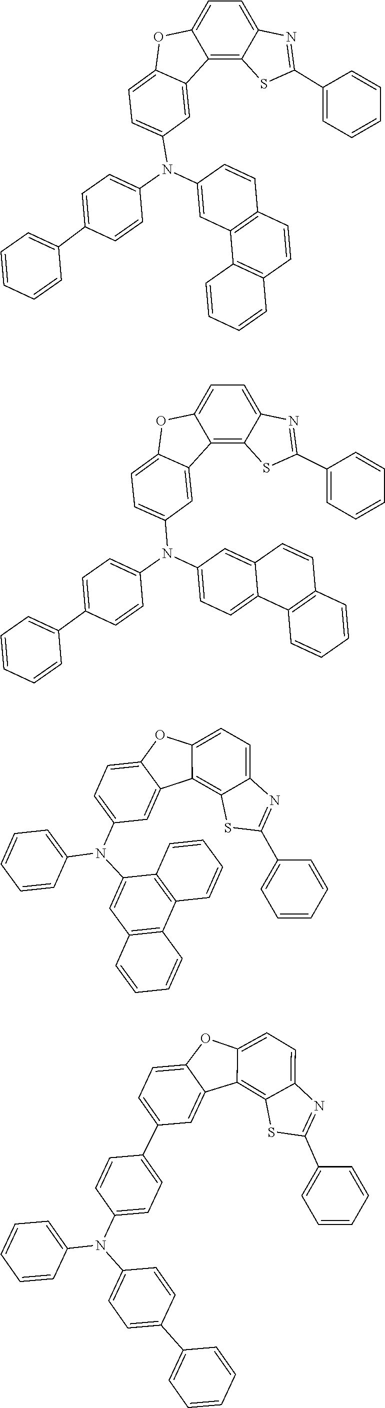 [SMILES: c1ccc(-c2ccc(N(c3ccc4ccc5ccccc5c4c3)c3ccc4oc5ccc6nc(-c7ccccc7)sc6c5c4c3)cc2)cc1 ; c1ccc(-c2ccc(N(c3ccc4c(ccc5ccccc54)c3)c3ccc4oc5ccc6nc(-c7ccccc7)sc6c5c4c3)cc2)cc1 ; c1ccc(-c2nc3ccc4oc5ccc(N(c6ccccc6)c6cc7ccccc7c7ccccc67)cc5c4c3s2)cc1 ; c1ccc(-c2ccc(N(c3ccccc3)c3ccc(-c4ccc5oc6ccc7nc(-c8ccccc8)sc7c6c5c4)cc3)cc2)cc1]

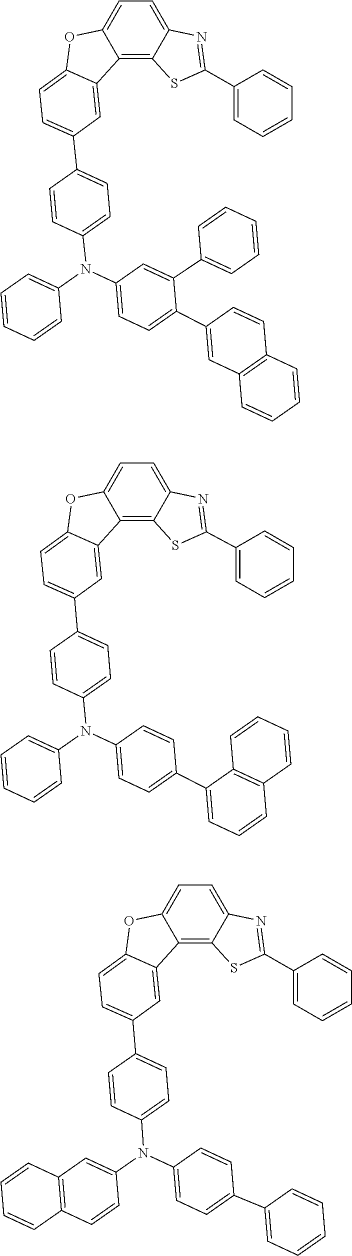 [SMILES: c1ccc(-c2nc3ccc4oc5ccc(-c6ccc(N(c7ccccc7)c7ccc(-c8ccc9ccccc9c8)c(-c8ccccc8)c7)cc6)cc5c4c3s2)cc1 ; c1ccc(-c2nc3ccc4oc5ccc(-c6ccc(N(c7ccccc7)c7ccc(-c8cccc9ccccc89)cc7)cc6)cc5c4c3s2)cc1 ; c1ccc(-c2ccc(N(c3ccc(-c4ccc5oc6ccc7nc(-c8ccccc8)sc7c6c5c4)cc3)c3ccc4ccccc4c3)cc2)cc1]

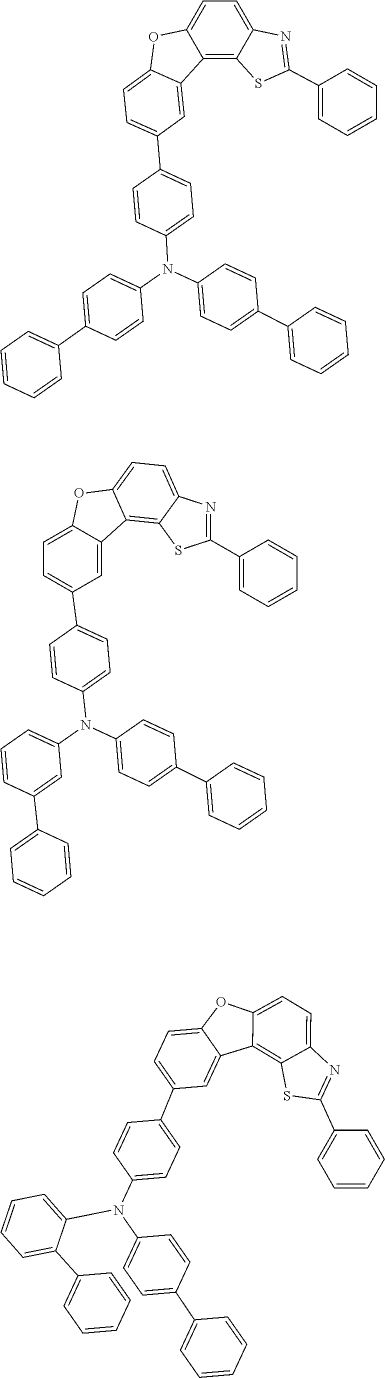 [SMILES: c1ccc(-c2ccc(N(c3ccc(-c4ccccc4)cc3)c3ccc(-c4ccc5oc6ccc7nc(-c8ccccc8)sc7c6c5c4)cc3)cc2)cc1 ; c1ccc(-c2ccc(N(c3ccc(-c4ccc5oc6ccc7nc(-c8ccccc8)sc7c6c5c4)cc3)c3cccc(-c4ccccc4)c3)cc2)cc1 ; c1ccc(-c2ccc(N(c3ccc(-c4ccc5oc6ccc7nc(-c8ccccc8)sc7c6c5c4)cc3)c3ccccc3-c3ccccc3)cc2)cc1]

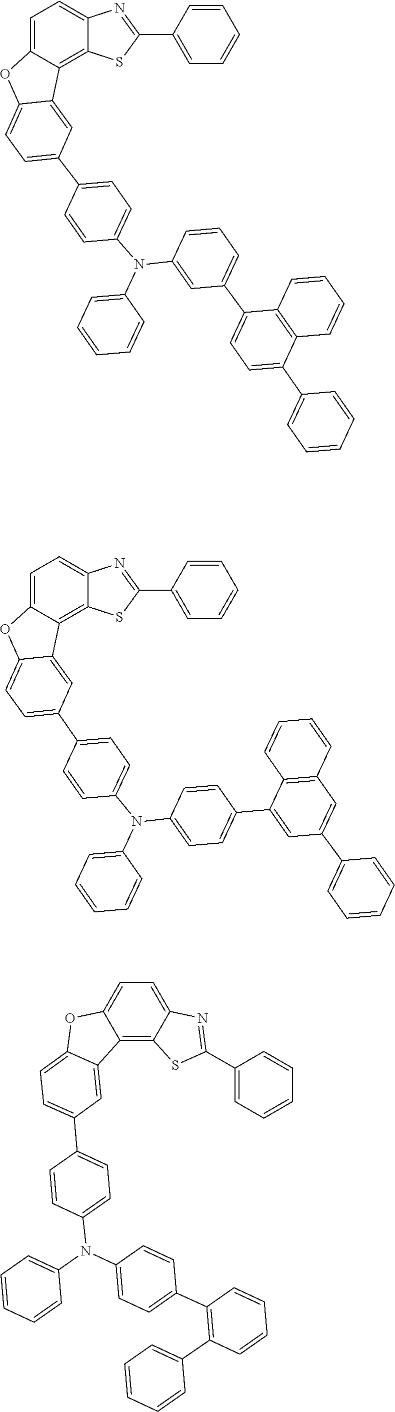 [SMILES: c1ccc(-c2nc3ccc4oc5ccc(-c6ccc(N(c7ccccc7)c7cccc(-c8ccc(-c9ccccc9)c9ccccc89)c7)cc6)cc5c4c3s2)cc1 ; c1ccc(-c2cc(-c3ccc(N(c4ccccc4)c4ccc(-c5ccc6oc7ccc8nc(-c9ccccc9)sc8c7c6c5)cc4)cc3)c3ccccc3c2)cc1 ; c1ccc(-c2nc3ccc4oc5ccc(-c6ccc(N(c7ccccc7)c7ccc(-c8ccccc8-c8ccccc8)cc7)cc6)cc5c4c3s2)cc1]

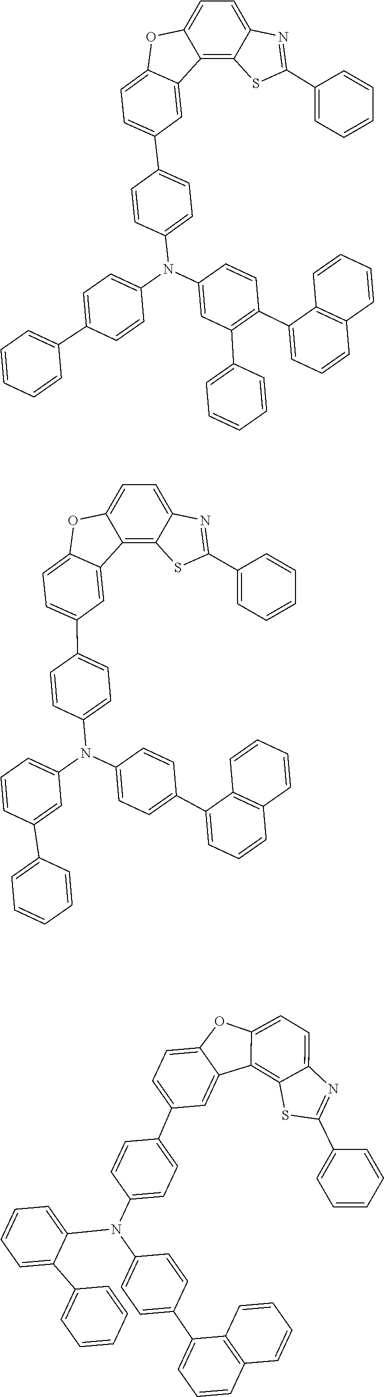 [SMILES: c1ccc(-c2ccc(N(c3ccc(-c4ccc5oc6ccc7nc(-c8ccccc8)sc7c6c5c4)cc3)c3ccc(-c4cccc5ccccc45)c(-c4ccccc4)c3)cc2)cc1 ; c1ccc(-c2cccc(N(c3ccc(-c4ccc5oc6ccc7nc(-c8ccccc8)sc7c6c5c4)cc3)c3ccc(-c4cccc5ccccc45)cc3)c2)cc1 ; c1ccc(-c2nc3ccc4oc5ccc(-c6ccc(N(c7ccc(-c8cccc9ccccc89)cc7)c7ccccc7-c7ccccc7)cc6)cc5c4c3s2)cc1]

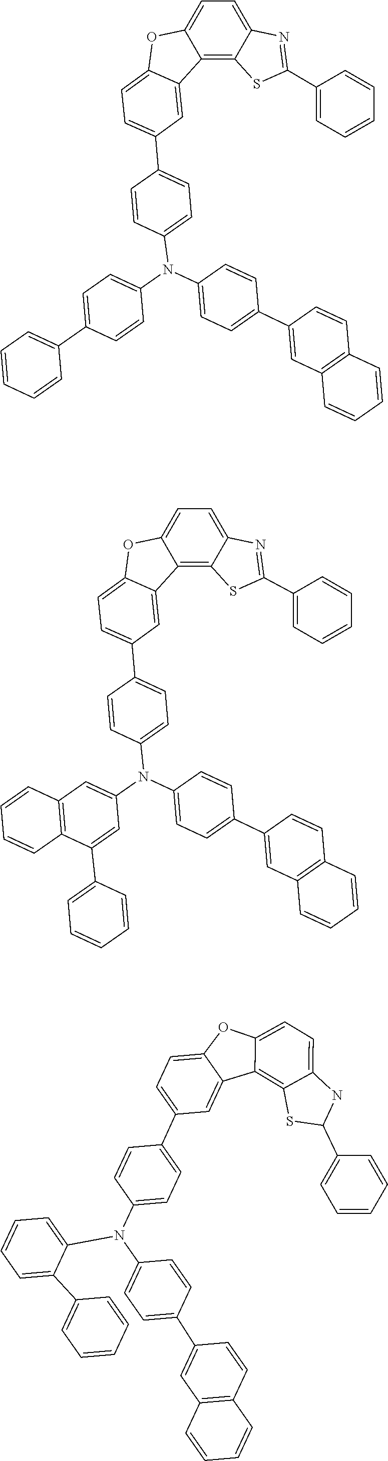

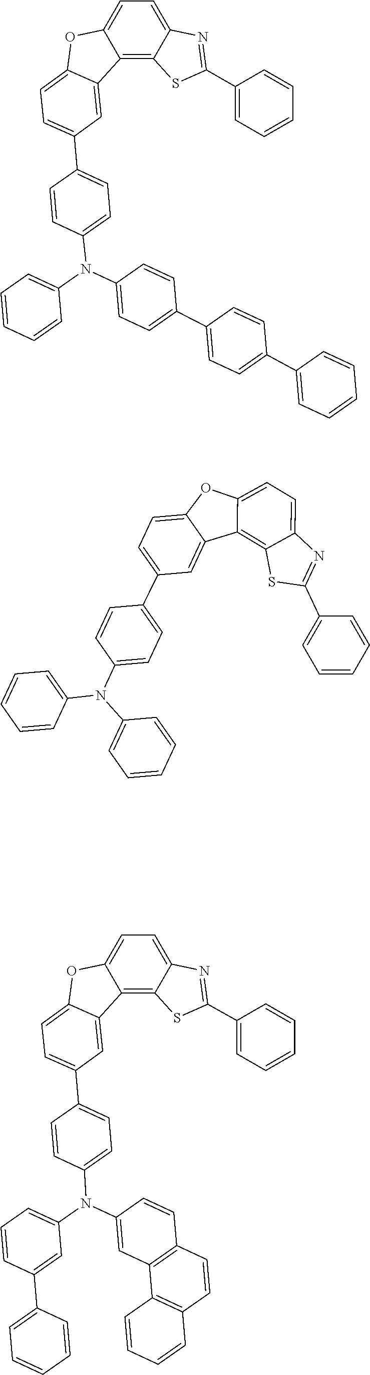 [SMILES: c1ccc(-c2ccc(-c3ccc(N(c4ccccc4)c4ccc(-c5ccc6oc7ccc8nc(-c9ccccc9)sc8c7c6c5)cc4)cc3)cc2)cc1 ; c1ccc(-c2nc3ccc4oc5ccc(-c6ccc(N(c7ccccc7)c7ccccc7)cc6)cc5c4c3s2)cc1 ; c1ccc(-c2cccc(N(c3ccc(-c4ccc5oc6ccc7nc(-c8ccccc8)sc7c6c5c4)cc3)c3ccc4ccc5ccccc5c4c3)c2)cc1]

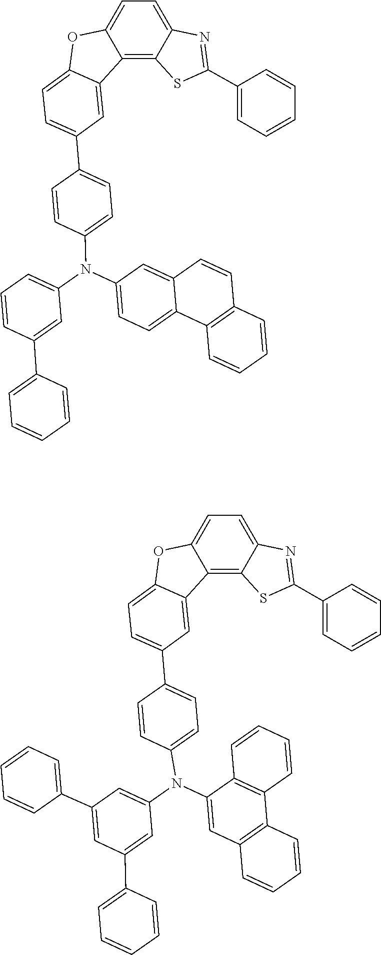 [SMILES: c1ccc(-c2cccc(N(c3ccc(-c4ccc5oc6ccc7nc(-c8ccccc8)sc7c6c5c4)cc3)c3ccc4c(ccc5ccccc54)c3)c2)cc1 ; c1ccc(-c2cc(-c3ccccc3)cc(N(c3ccc(-c4ccc5oc6ccc7nc(-c8ccccc8)sc7c6c5c4)cc3)c3cc4ccccc4c4ccccc34)c2)cc1]

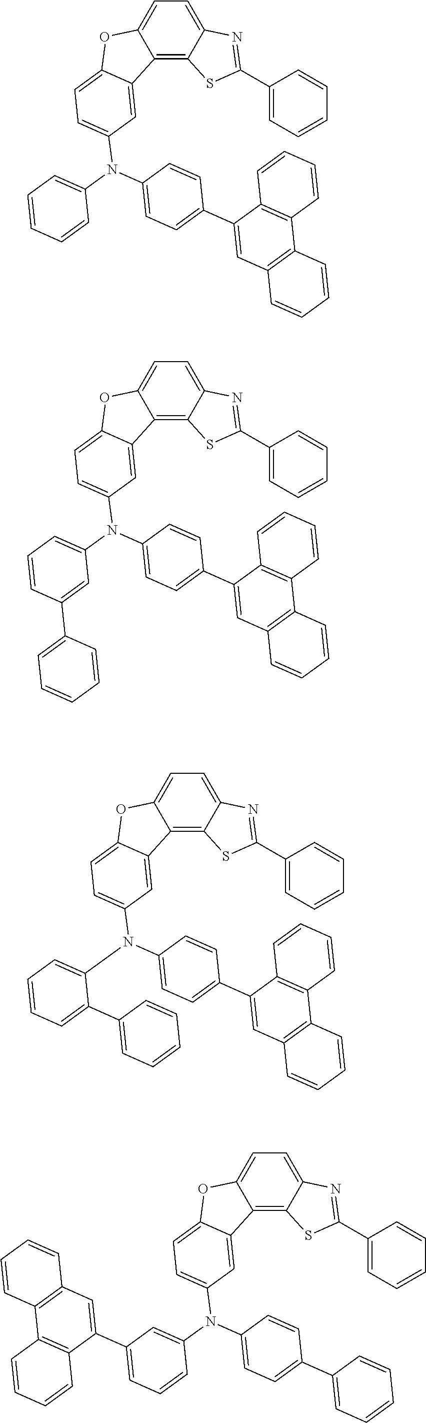 [SMILES: c1ccc(-c2nc3ccc4oc5ccc(N(c6ccccc6)c6ccc(-c7cc8ccccc8c8ccccc78)cc6)cc5c4c3s2)cc1 ; c1ccc(-c2cccc(N(c3ccc(-c4cc5ccccc5c5ccccc45)cc3)c3ccc4oc5ccc6nc(-c7ccccc7)sc6c5c4c3)c2)cc1 ; c1ccc(-c2nc3ccc4oc5ccc(N(c6ccc(-c7cc8ccccc8c8ccccc78)cc6)c6ccccc6-c6ccccc6)cc5c4c3s2)cc1 ; c1ccc(-c2ccc(N(c3cccc(-c4cc5ccccc5c5ccccc45)c3)c3ccc4oc5ccc6nc(-c7ccccc7)sc6c5c4c3)cc2)cc1]

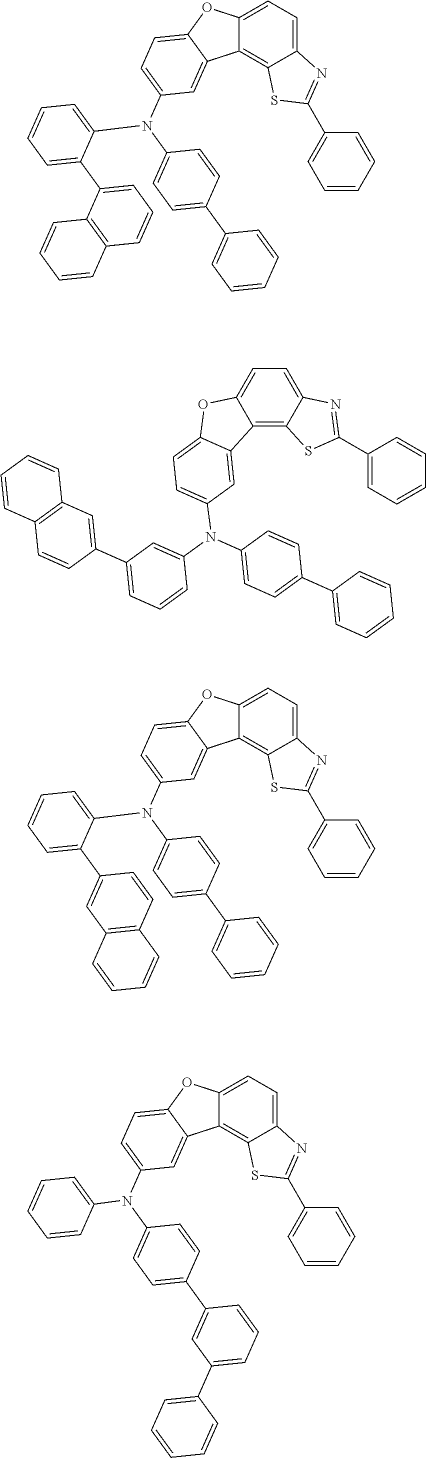 [SMILES: c1ccc(-c2ccc(N(c3ccc4oc5ccc6nc(-c7ccccc7)sc6c5c4c3)c3ccccc3-c3cccc4ccccc34)cc2)cc1 ; c1ccc(-c2ccc(N(c3cccc(-c4ccc5ccccc5c4)c3)c3ccc4oc5ccc6nc(-c7ccccc7)sc6c5c4c3)cc2)cc1 ; c1ccc(-c2ccc(N(c3ccc4oc5ccc6nc(-c7ccccc7)sc6c5c4c3)c3ccccc3-c3ccc4ccccc4c3)cc2)cc1 ; c1ccc(-c2cccc(-c3ccc(N(c4ccccc4)c4ccc5oc6ccc7nc(-c8ccccc8)sc7c6c5c4)cc3)c2)cc1]

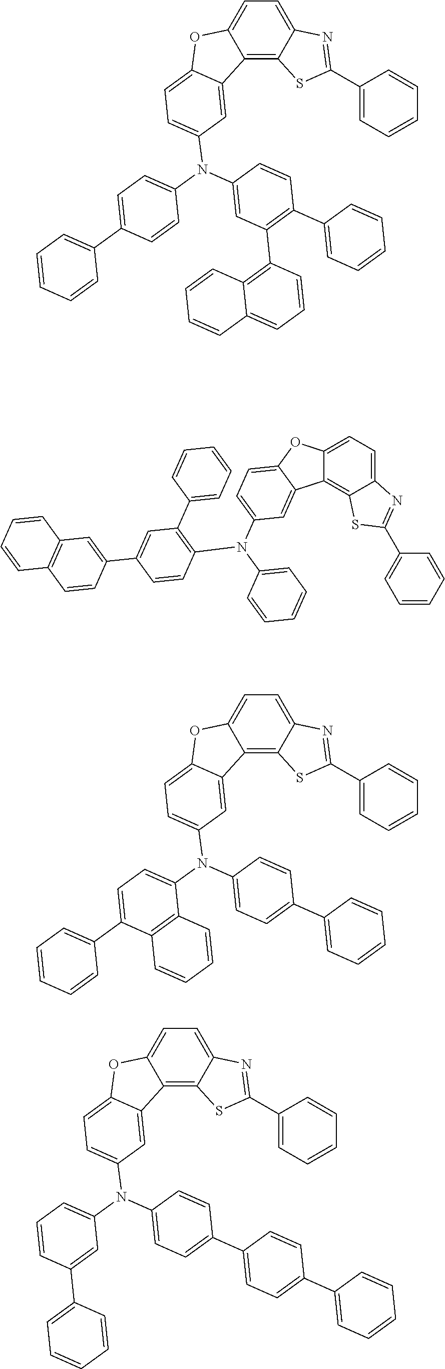 [SMILES: c1ccc(-c2ccc(N(c3ccc(-c4ccccc4)c(-c4cccc5ccccc45)c3)c3ccc4oc5ccc6nc(-c7ccccc7)sc6c5c4c3)cc2)cc1 ; c1ccc(-c2nc3ccc4oc5ccc(N(c6ccccc6)c6ccc(-c7ccc8ccccc8c7)cc6-c6ccccc6)cc5c4c3s2)cc1 ; c1ccc(-c2ccc(N(c3ccc4oc5ccc6nc(-c7ccccc7)sc6c5c4c3)c3ccc(-c4ccccc4)c4ccccc34)cc2)cc1 ; c1ccc(-c2ccc(-c3ccc(N(c4cccc(-c5ccccc5)c4)c4ccc5oc6ccc7nc(-c8ccccc8)sc7c6c5c4)cc3)cc2)cc1]

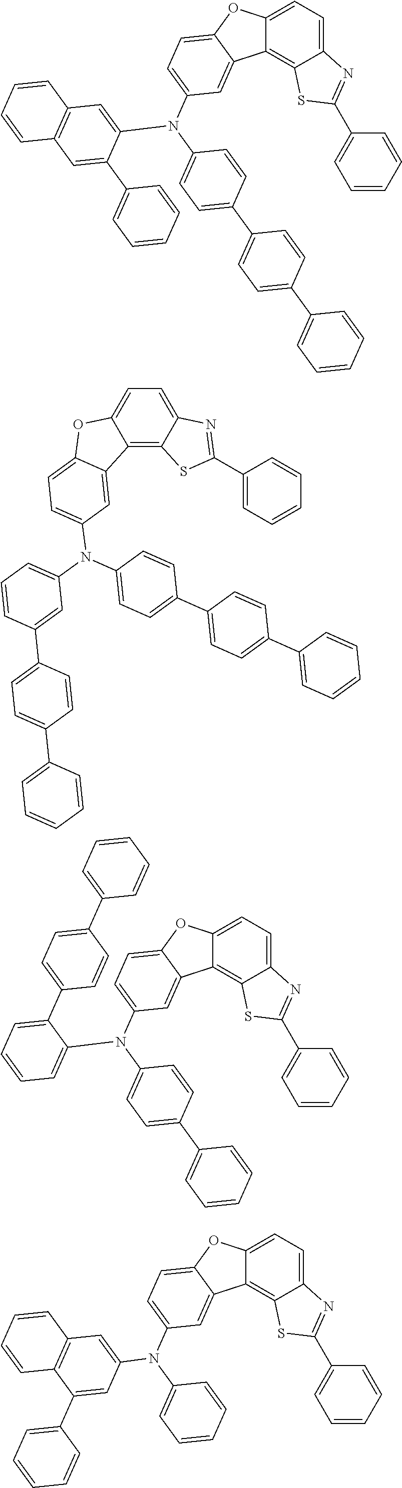

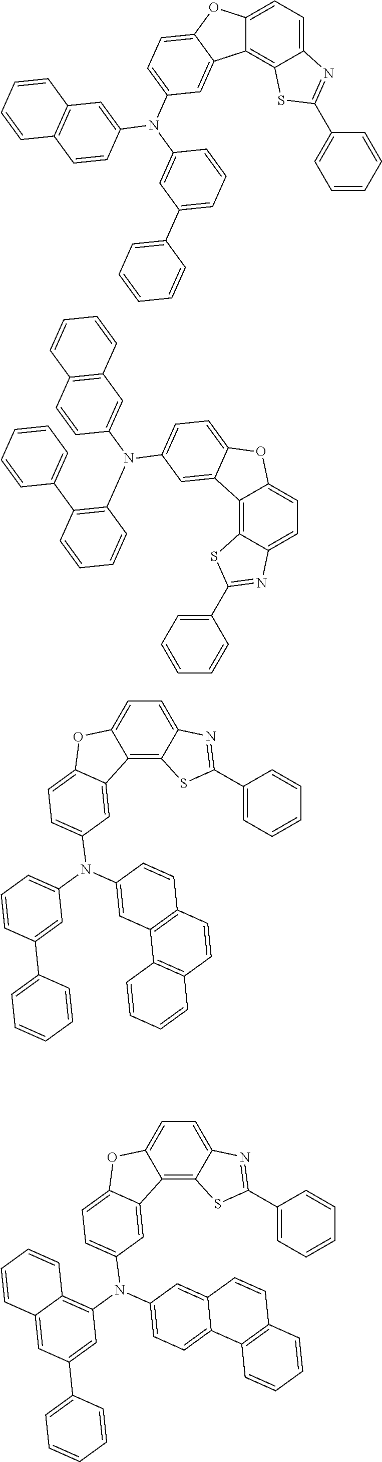

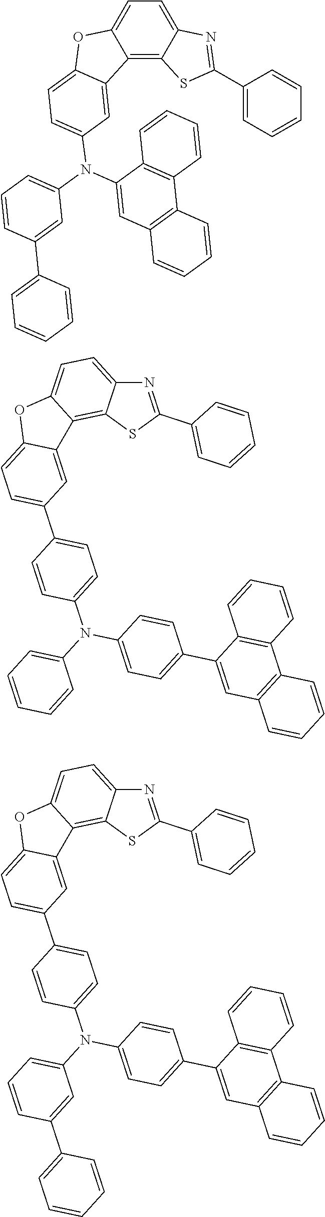

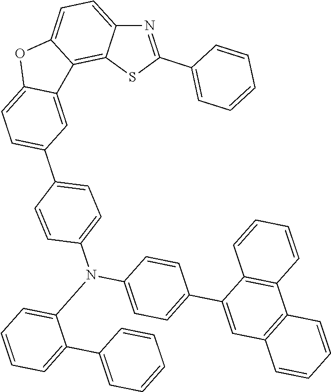 [SMILES: c1ccc(-c2nc3ccc4oc5ccc(-c6ccc(N(c7ccc(-c8cc9ccccc9c9ccccc89)cc7)c7ccccc7-c7ccccc7)cc6)cc5c4c3s2)cc1]

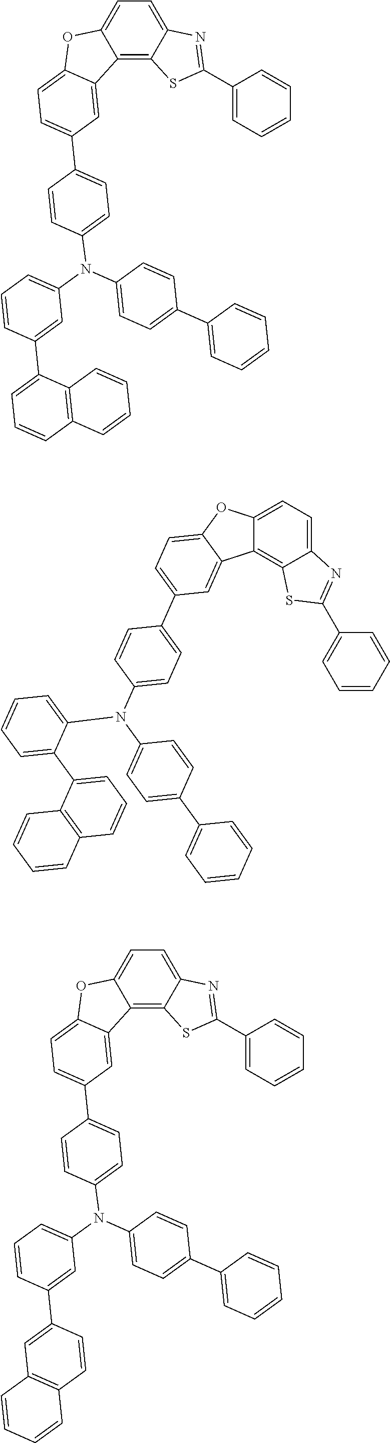 [SMILES: c1ccc(-c2ccc(N(c3ccc(-c4ccc5oc6ccc7nc(-c8ccccc8)sc7c6c5c4)cc3)c3cccc(-c4cccc5ccccc45)c3)cc2)cc1 ; c1ccc(-c2ccc(N(c3ccc(-c4ccc5oc6ccc7nc(-c8ccccc8)sc7c6c5c4)cc3)c3ccccc3-c3cccc4ccccc34)cc2)cc1 ; c1ccc(-c2ccc(N(c3ccc(-c4ccc5oc6ccc7nc(-c8ccccc8)sc7c6c5c4)cc3)c3cccc(-c4ccc5ccccc5c4)c3)cc2)cc1]

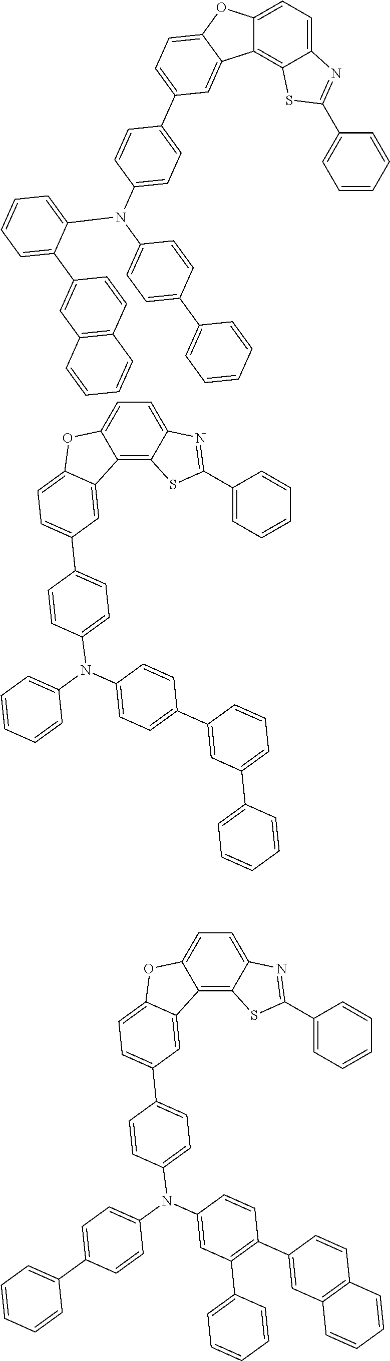

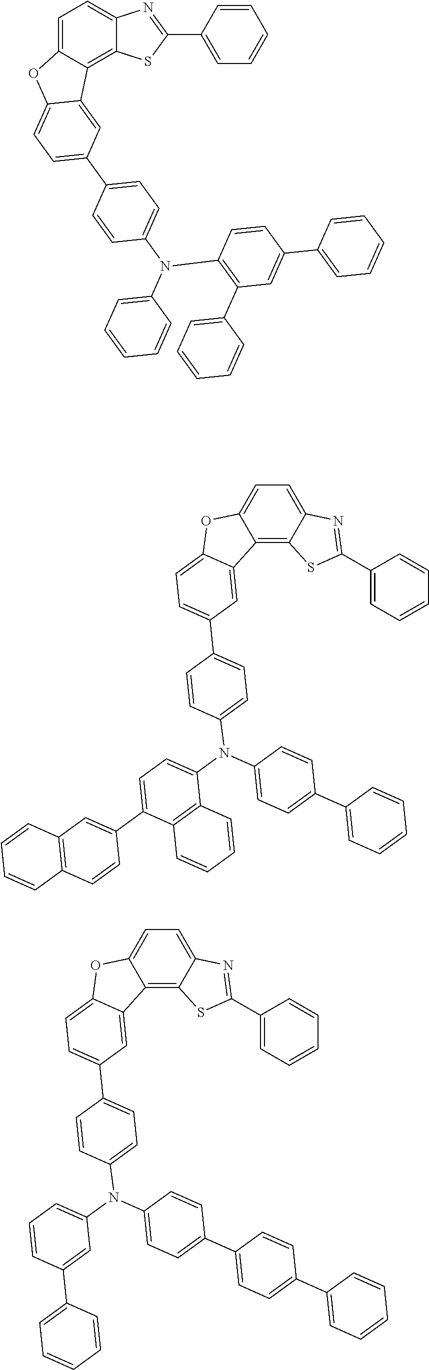 [SMILES: c1ccc(-c2ccc(N(c3ccccc3)c3ccc(-c4ccc5oc6ccc7nc(-c8ccccc8)sc7c6c5c4)cc3)c(-c3ccccc3)c2)cc1 ; c1ccc(-c2ccc(N(c3ccc(-c4ccc5oc6ccc7nc(-c8ccccc8)sc7c6c5c4)cc3)c3ccc(-c4ccc5ccccc5c4)c4ccccc34)cc2)cc1 ; c1ccc(-c2ccc(-c3ccc(N(c4ccc(-c5ccc6oc7ccc8nc(-c9ccccc9)sc8c7c6c5)cc4)c4cccc(-c5ccccc5)c4)cc3)cc2)cc1]

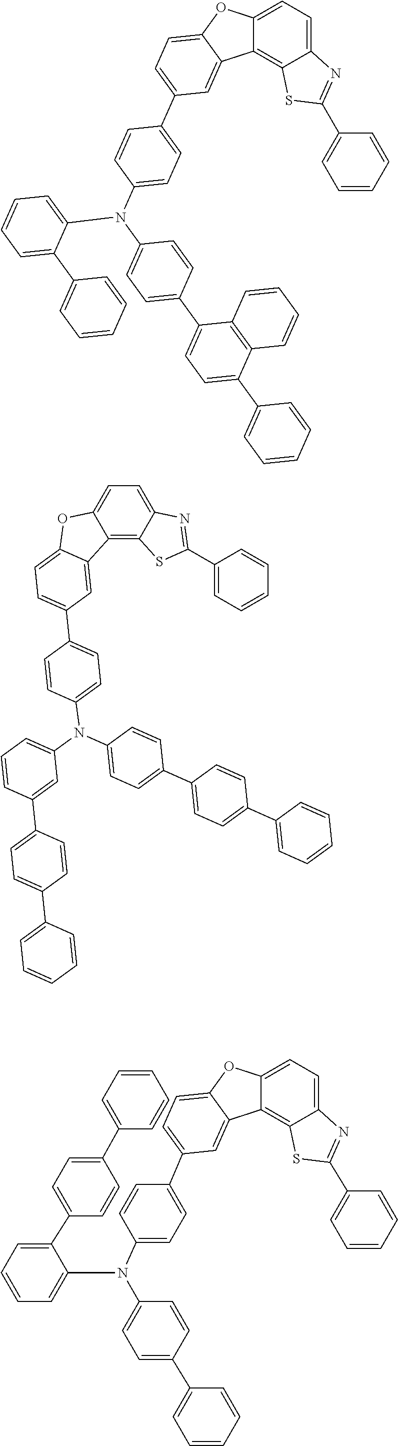 [SMILES: c1ccc(-c2nc3ccc4oc5ccc(-c6ccc(N(c7ccc(-c8ccc(-c9ccccc9)c9ccccc89)cc7)c7ccccc7-c7ccccc7)cc6)cc5c4c3s2)cc1 ; c1ccc(-c2ccc(-c3ccc(N(c4ccc(-c5ccc6oc7ccc8nc(-c9ccccc9)sc8c7c6c5)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4ccc5oc6ccc7nc(-c8ccccc8)sc7c6c5c4)cc3)cc2)cc1]

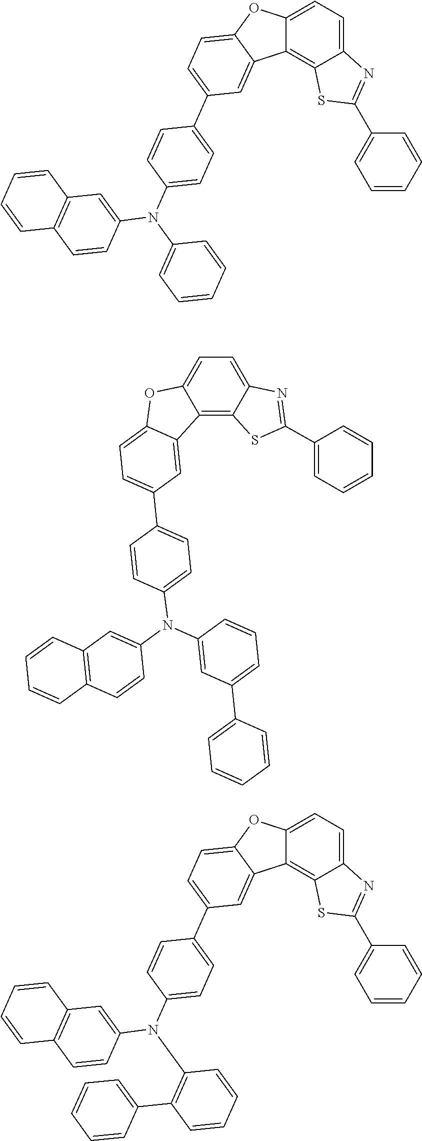

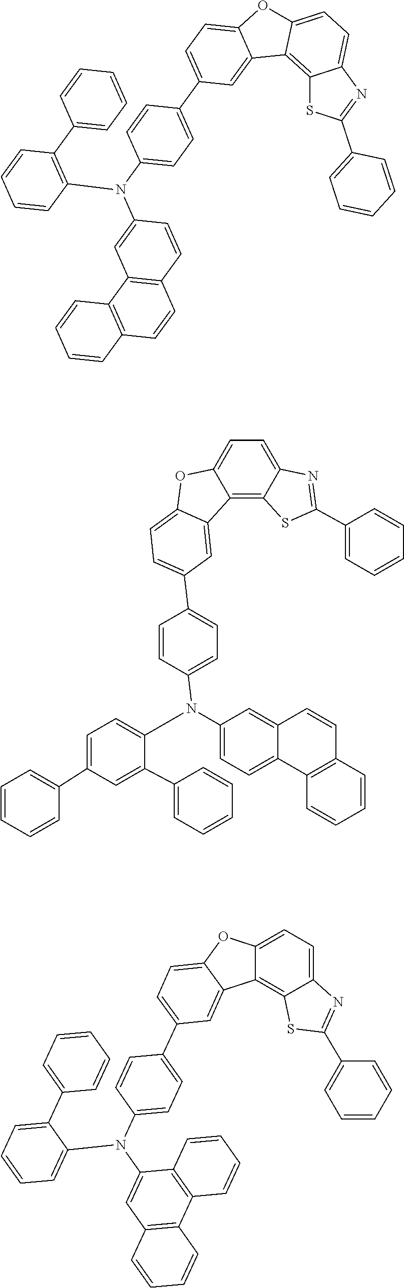 [SMILES: c1ccc(-c2nc3ccc4oc5ccc(-c6ccc(N(c7ccc8ccc9ccccc9c8c7)c7ccccc7-c7ccccc7)cc6)cc5c4c3s2)cc1 ; c1ccc(-c2ccc(N(c3ccc(-c4ccc5oc6ccc7nc(-c8ccccc8)sc7c6c5c4)cc3)c3ccc4c(ccc5ccccc54)c3)c(-c3ccccc3)c2)cc1 ; c1ccc(-c2nc3ccc4oc5ccc(-c6ccc(N(c7ccccc7-c7ccccc7)c7cc8ccccc8c8ccccc78)cc6)cc5c4c3s2)cc1]

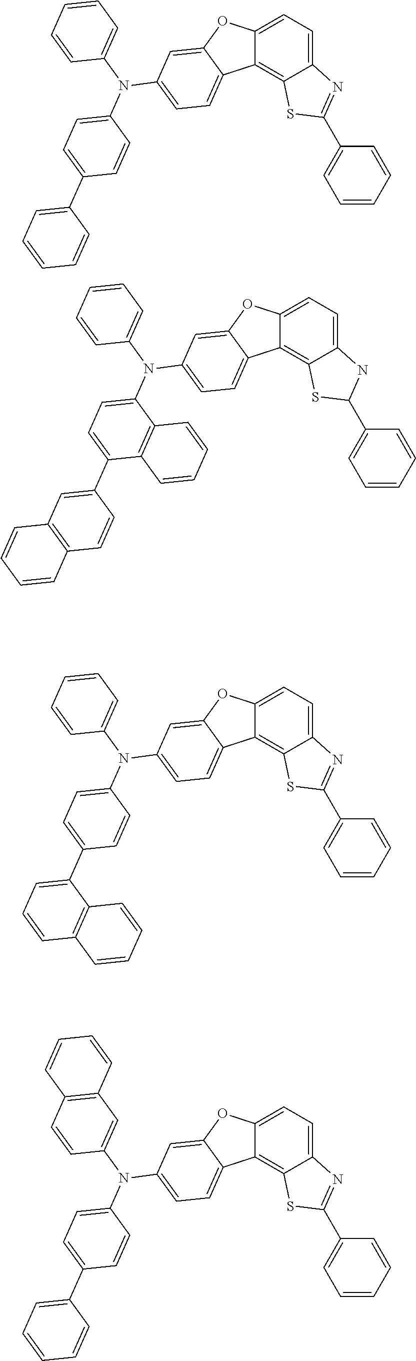

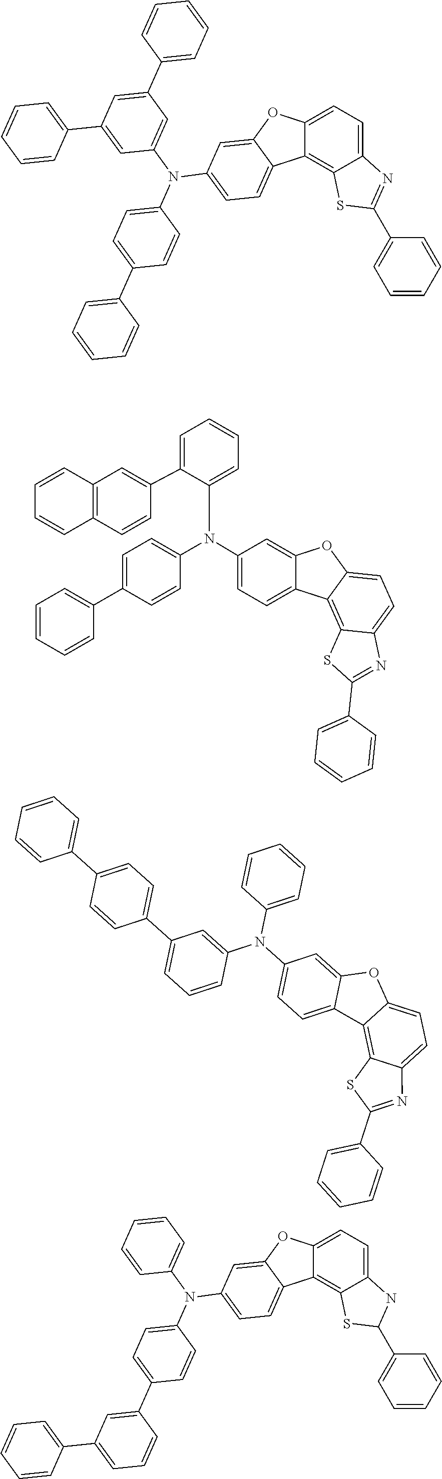

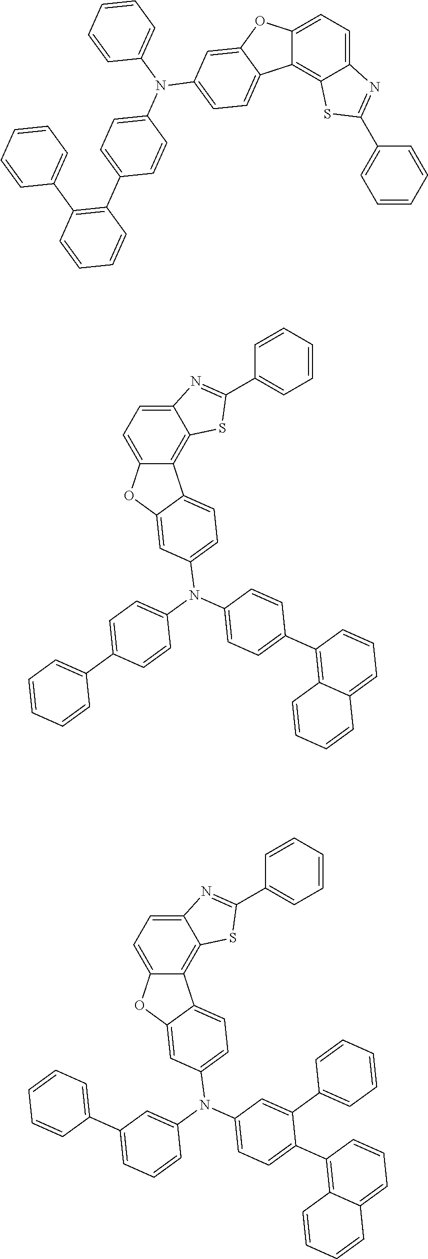 [SMILES: c1ccc(-c2nc3ccc4oc5cc(N(c6ccccc6)c6ccc(-c7ccccc7-c7ccccc7)cc6)ccc5c4c3s2)cc1 ; c1ccc(-c2ccc(N(c3ccc(-c4cccc5ccccc45)cc3)c3ccc4c(c3)oc3ccc5nc(-c6ccccc6)sc5c34)cc2)cc1 ; c1ccc(-c2cccc(N(c3ccc(-c4cccc5ccccc45)c(-c4ccccc4)c3)c3ccc4c(c3)oc3ccc5nc(-c6ccccc6)sc5c34)c2)cc1]

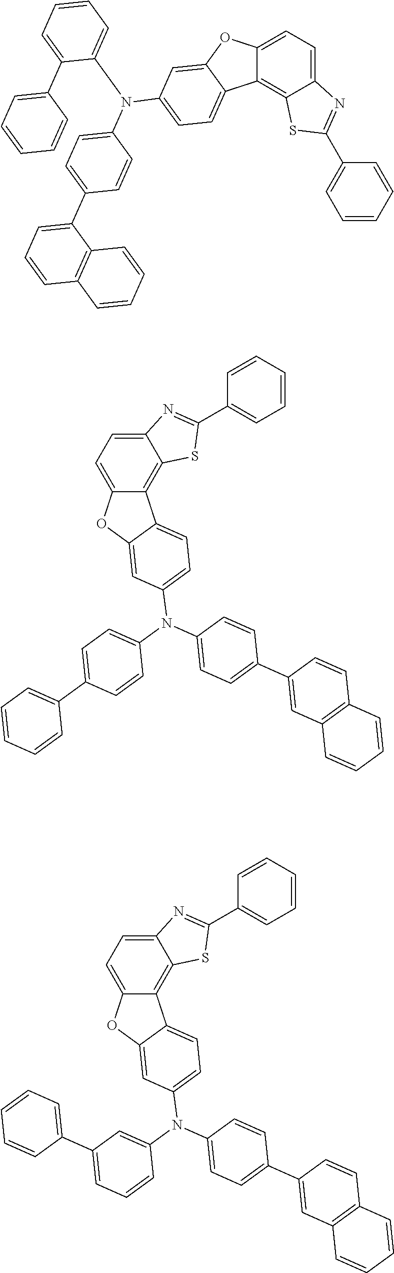 [SMILES: c1ccc(-c2nc3ccc4oc5cc(N(c6ccc(-c7cccc8ccccc78)cc6)c6ccccc6-c6ccccc6)ccc5c4c3s2)cc1 ; c1ccc(-c2ccc(N(c3ccc(-c4ccc5ccccc5c4)cc3)c3ccc4c(c3)oc3ccc5nc(-c6ccccc6)sc5c34)cc2)cc1 ; c1ccc(-c2cccc(N(c3ccc(-c4ccc5ccccc5c4)cc3)c3ccc4c(c3)oc3ccc5nc(-c6ccccc6)sc5c34)c2)cc1]

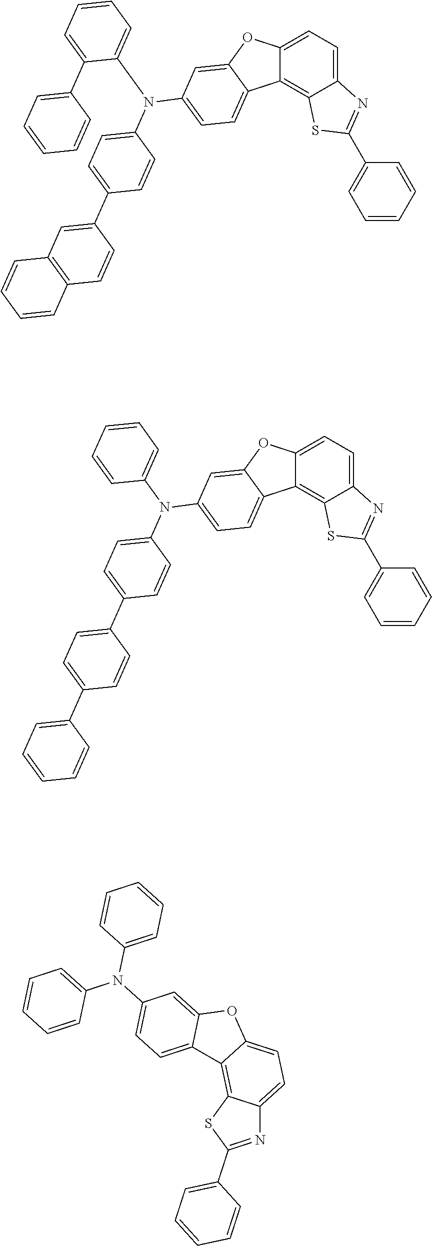 [SMILES: c1ccc(-c2nc3ccc4oc5cc(N(c6ccc(-c7ccc8ccccc8c7)cc6)c6ccccc6-c6ccccc6)ccc5c4c3s2)cc1 ; c1ccc(-c2ccc(-c3ccc(N(c4ccccc4)c4ccc5c(c4)oc4ccc6nc(-c7ccccc7)sc6c45)cc3)cc2)cc1 ; c1ccc(-c2nc3ccc4oc5cc(N(c6ccccc6)c6ccccc6)ccc5c4c3s2)cc1]

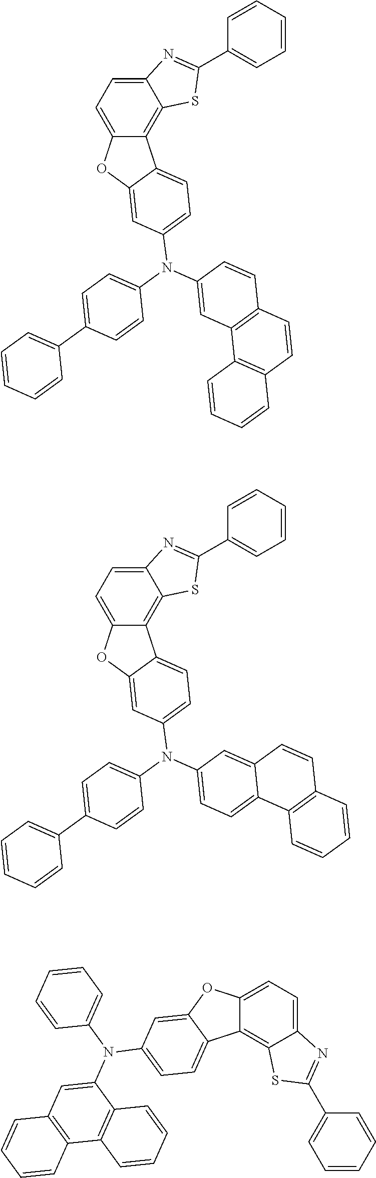 [SMILES: c1ccc(-c2ccc(N(c3ccc4c(c3)oc3ccc5nc(-c6ccccc6)sc5c34)c3ccc4ccc5ccccc5c4c3)cc2)cc1 ; c1ccc(-c2ccc(N(c3ccc4c(ccc5ccccc54)c3)c3ccc4c(c3)oc3ccc5nc(-c6ccccc6)sc5c34)cc2)cc1 ; c1ccc(-c2nc3ccc4oc5cc(N(c6ccccc6)c6cc7ccccc7c7ccccc67)ccc5c4c3s2)cc1]

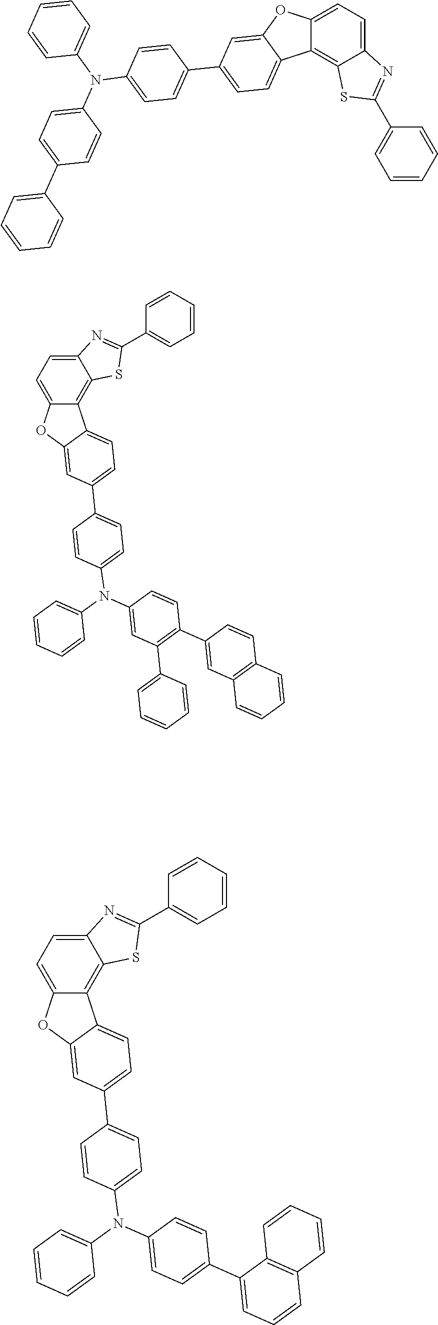 [SMILES: c1ccc(-c2ccc(N(c3ccccc3)c3ccc(-c4ccc5c(c4)oc4ccc6nc(-c7ccccc7)sc6c45)cc3)cc2)cc1 ; c1ccc(-c2nc3ccc4oc5cc(-c6ccc(N(c7ccccc7)c7ccc(-c8ccc9ccccc9c8)c(-c8ccccc8)c7)cc6)ccc5c4c3s2)cc1 ; c1ccc(-c2nc3ccc4oc5cc(-c6ccc(N(c7ccccc7)c7ccc(-c8cccc9ccccc89)cc7)cc6)ccc5c4c3s2)cc1]

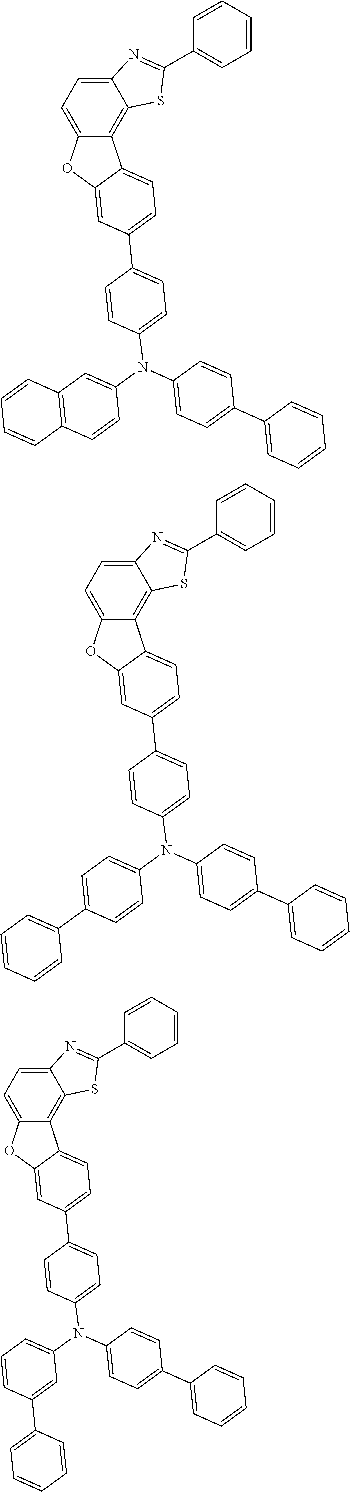

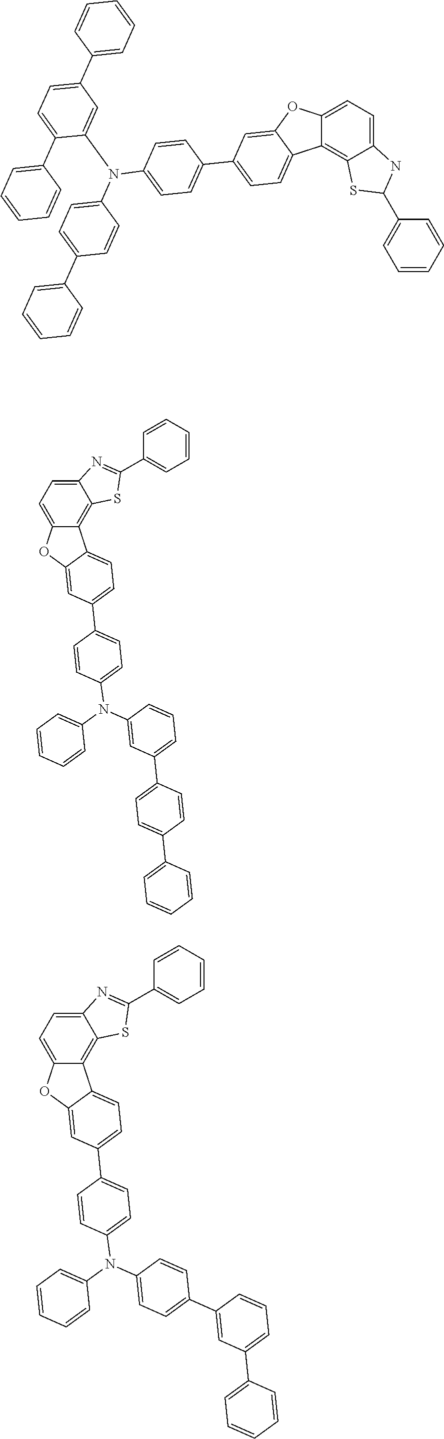

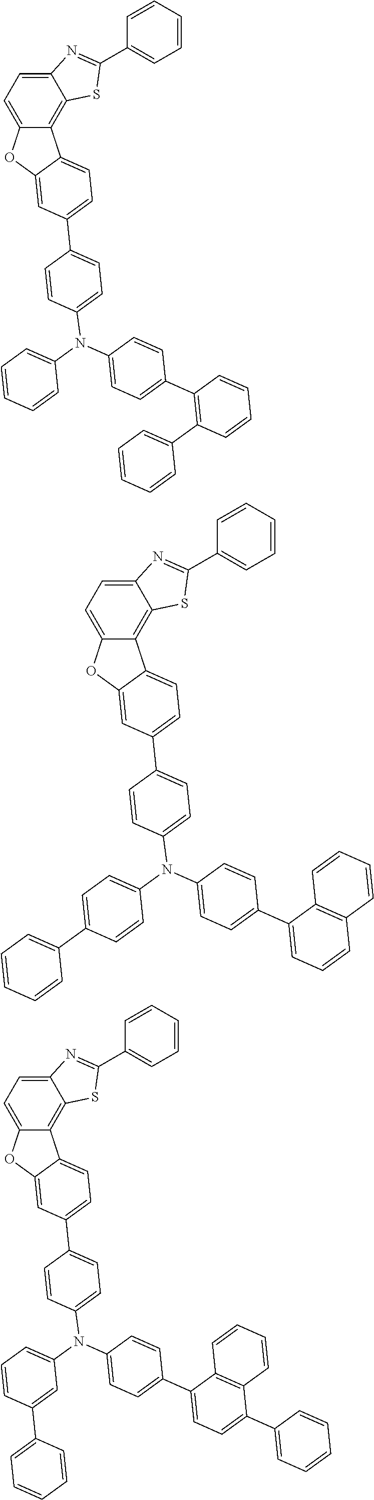 [SMILES: c1ccc(-c2nc3ccc4oc5cc(-c6ccc(N(c7ccccc7)c7ccc(-c8ccccc8-c8ccccc8)cc7)cc6)ccc5c4c3s2)cc1 ; c1ccc(-c2ccc(N(c3ccc(-c4ccc5c(c4)oc4ccc6nc(-c7ccccc7)sc6c45)cc3)c3ccc(-c4cccc5ccccc45)cc3)cc2)cc1 ; c1ccc(-c2cccc(N(c3ccc(-c4ccc5c(c4)oc4ccc6nc(-c7ccccc7)sc6c45)cc3)c3ccc(-c4ccc(-c5ccccc5)c5ccccc45)cc3)c2)cc1]

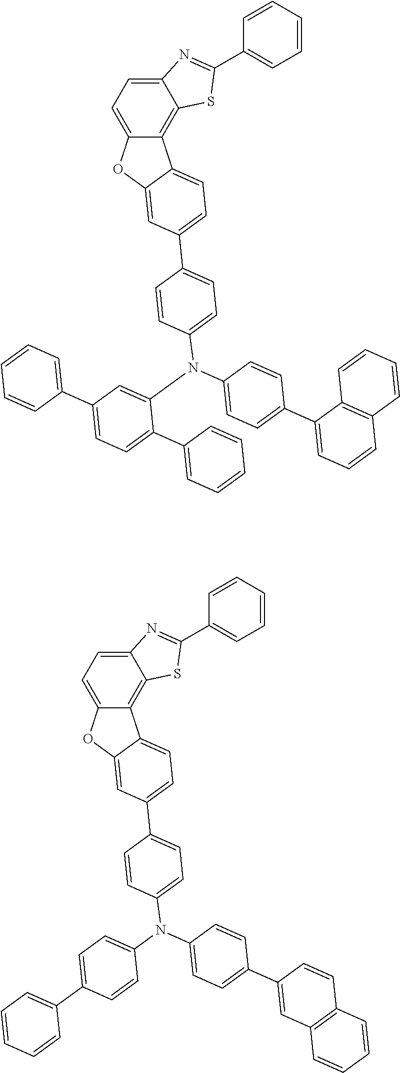 [SMILES: c1ccc(-c2ccc(-c3ccccc3)c(N(c3ccc(-c4ccc5c(c4)oc4ccc6nc(-c7ccccc7)sc6c45)cc3)c3ccc(-c4cccc5ccccc45)cc3)c2)cc1 ; c1ccc(-c2ccc(N(c3ccc(-c4ccc5ccccc5c4)cc3)c3ccc(-c4ccc5c(c4)oc4ccc6nc(-c7ccccc7)sc6c45)cc3)cc2)cc1]

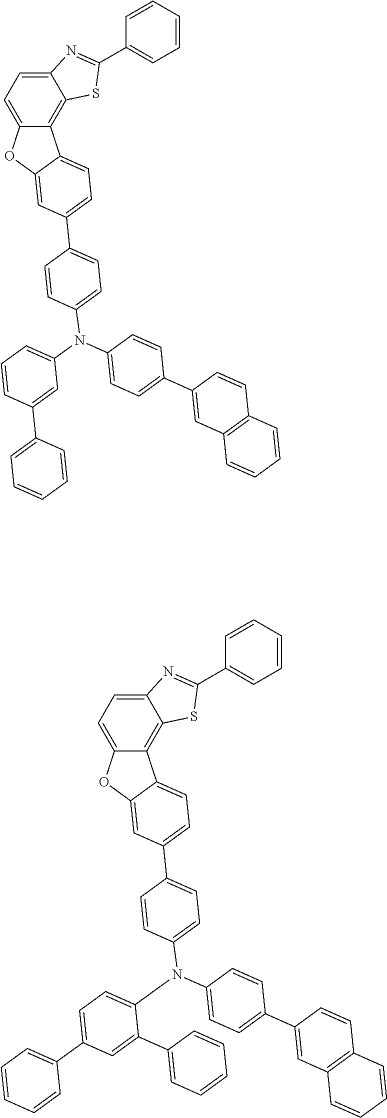 [SMILES: c1ccc(-c2cccc(N(c3ccc(-c4ccc5ccccc5c4)cc3)c3ccc(-c4ccc5c(c4)oc4ccc6nc(-c7ccccc7)sc6c45)cc3)c2)cc1 ; c1ccc(-c2ccc(N(c3ccc(-c4ccc5ccccc5c4)cc3)c3ccc(-c4ccc5c(c4)oc4ccc6nc(-c7ccccc7)sc6c45)cc3)c(-c3ccccc3)c2)cc1]

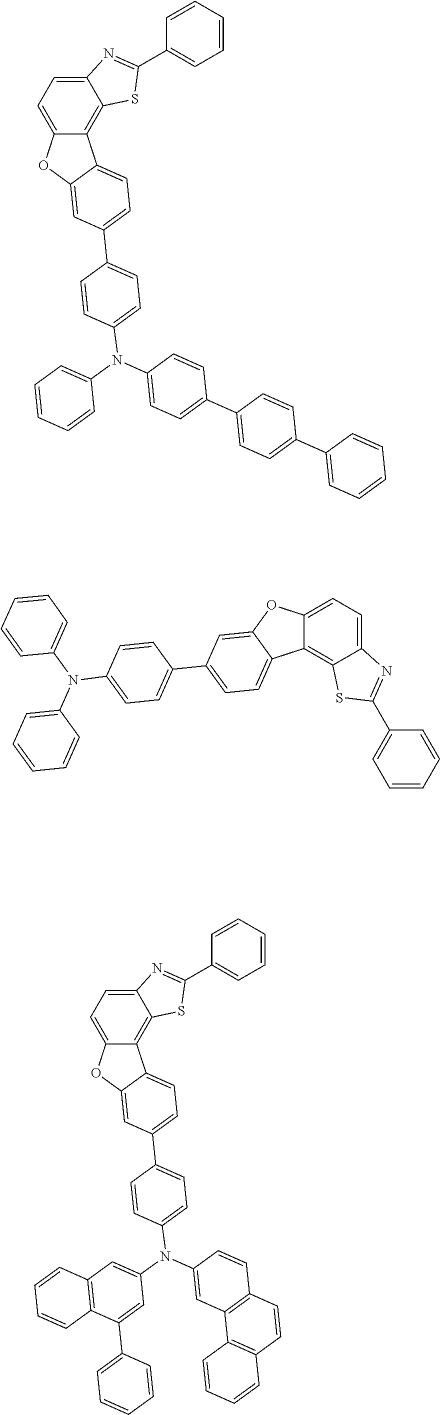 [SMILES: c1ccc(-c2ccc(-c3ccc(N(c4ccccc4)c4ccc(-c5ccc6c(c5)oc5ccc7nc(-c8ccccc8)sc7c56)cc4)cc3)cc2)cc1 ; c1ccc(-c2nc3ccc4oc5cc(-c6ccc(N(c7ccccc7)c7ccccc7)cc6)ccc5c4c3s2)cc1 ; c1ccc(-c2nc3ccc4oc5cc(-c6ccc(N(c7cc(-c8ccccc8)c8ccccc8c7)c7ccc8ccc9ccccc9c8c7)cc6)ccc5c4c3s2)cc1]

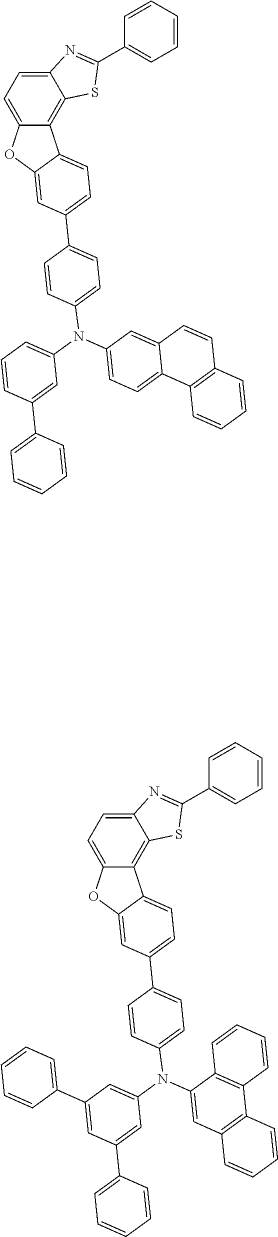 [SMILES: c1ccc(-c2cccc(N(c3ccc(-c4ccc5c(c4)oc4ccc6nc(-c7ccccc7)sc6c45)cc3)c3ccc4c(ccc5ccccc54)c3)c2)cc1 ; c1ccc(-c2cc(-c3ccccc3)cc(N(c3ccc(-c4ccc5c(c4)oc4ccc6nc(-c7ccccc7)sc6c45)cc3)c3cc4ccccc4c4ccccc34)c2)cc1]

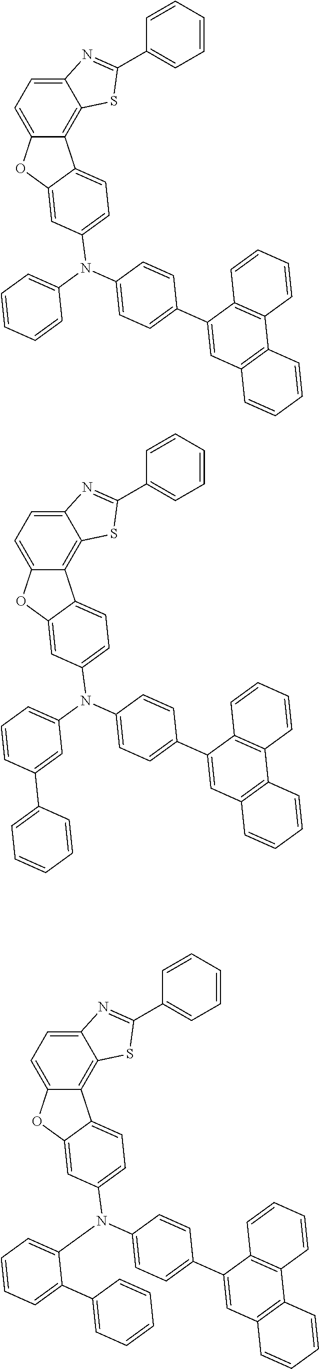 [SMILES: c1ccc(-c2nc3ccc4oc5cc(N(c6ccccc6)c6ccc(-c7cc8ccccc8c8ccccc78)cc6)ccc5c4c3s2)cc1 ; c1ccc(-c2cccc(N(c3ccc(-c4cc5ccccc5c5ccccc45)cc3)c3ccc4c(c3)oc3ccc5nc(-c6ccccc6)sc5c34)c2)cc1 ; c1ccc(-c2nc3ccc4oc5cc(N(c6ccc(-c7cc8ccccc8c8ccccc78)cc6)c6ccccc6-c6ccccc6)ccc5c4c3s2)cc1]

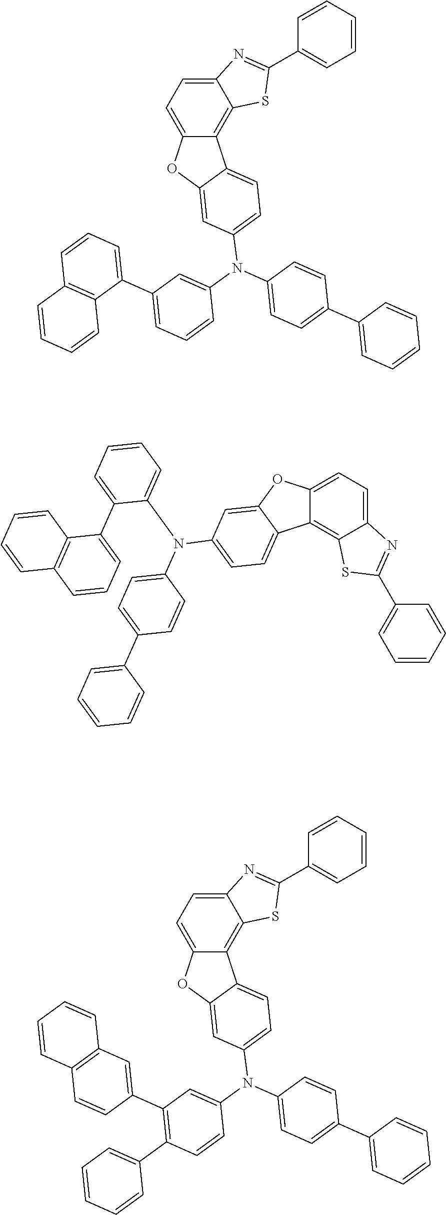 [SMILES: c1ccc(-c2ccc(N(c3cccc(-c4cccc5ccccc45)c3)c3ccc4c(c3)oc3ccc5nc(-c6ccccc6)sc5c34)cc2)cc1 ; c1ccc(-c2ccc(N(c3ccc4c(c3)oc3ccc5nc(-c6ccccc6)sc5c34)c3ccccc3-c3cccc4ccccc34)cc2)cc1 ; c1ccc(-c2ccc(N(c3ccc(-c4ccccc4)c(-c4ccc5ccccc5c4)c3)c3ccc4c(c3)oc3ccc5nc(-c6ccccc6)sc5c34)cc2)cc1]

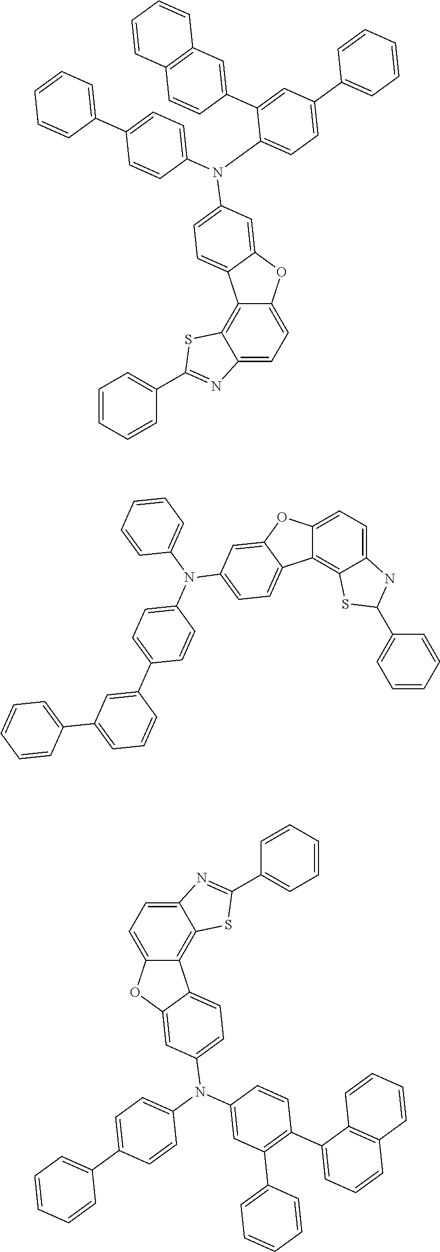

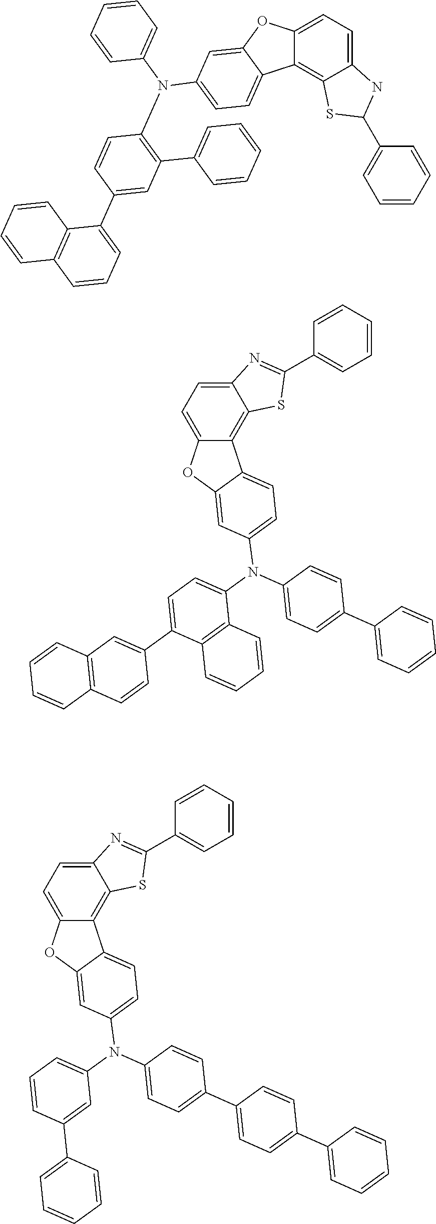

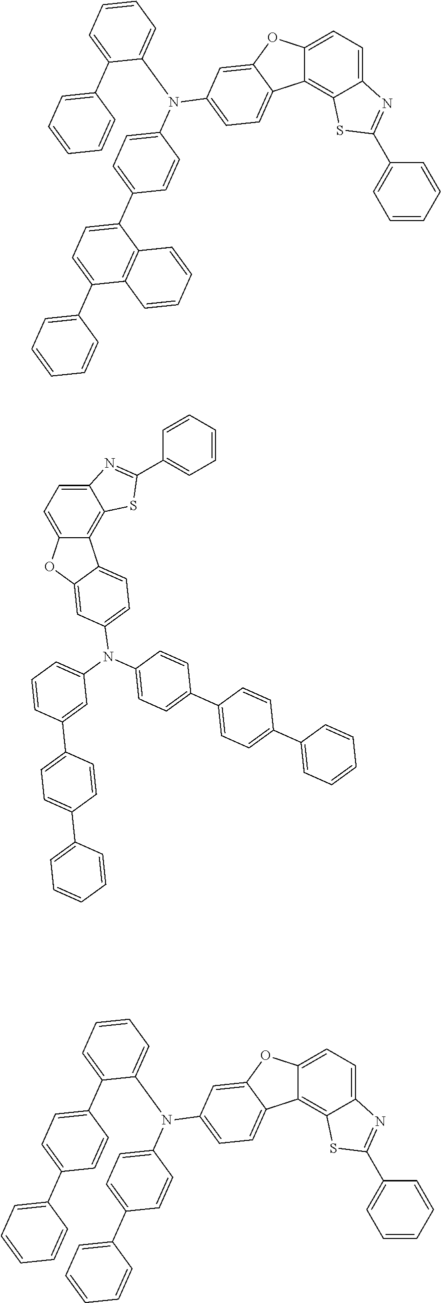 [SMILES: c1ccc(-c2nc3ccc4oc5cc(N(c6ccc(-c7ccc(-c8ccccc8)c8ccccc78)cc6)c6ccccc6-c6ccccc6)ccc5c4c3s2)cc1 ; c1ccc(-c2ccc(-c3ccc(N(c4cccc(-c5ccc(-c6ccccc6)cc5)c4)c4ccc5c(c4)oc4ccc6nc(-c7ccccc7)sc6c45)cc3)cc2)cc1 ; c1ccc(-c2ccc(-c3ccccc3N(c3ccc(-c4ccccc4)cc3)c3ccc4c(c3)oc3ccc5nc(-c6ccccc6)sc5c34)cc2)cc1]

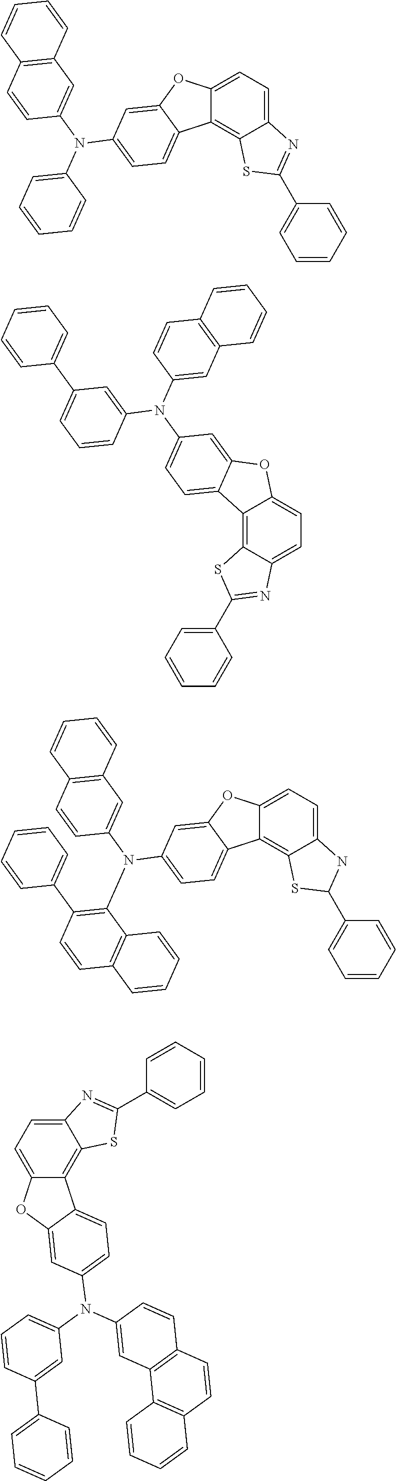

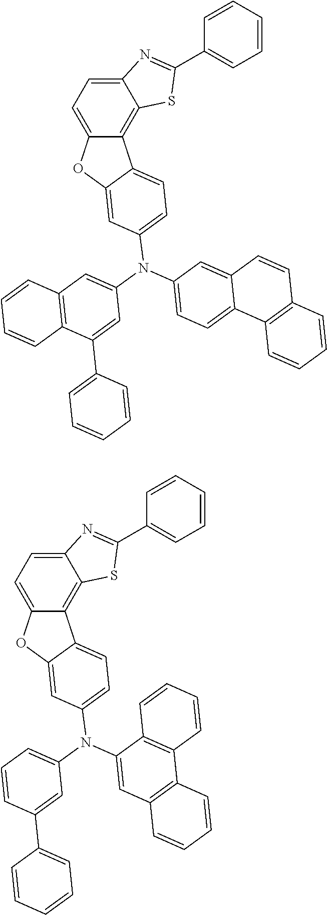 [SMILES: c1ccc(-c2nc3ccc4oc5cc(N(c6cc(-c7ccccc7)c7ccccc7c6)c6ccc7c(ccc8ccccc87)c6)ccc5c4c3s2)cc1 ; c1ccc(-c2cccc(N(c3ccc4c(c3)oc3ccc5nc(-c6ccccc6)sc5c34)c3cc4ccccc4c4ccccc34)c2)cc1]

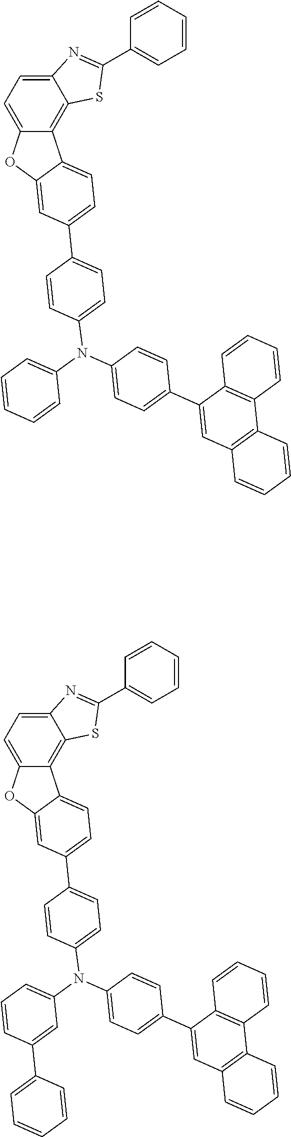 [SMILES: c1ccc(-c2nc3ccc4oc5cc(-c6ccc(N(c7ccccc7)c7ccc(-c8cc9ccccc9c9ccccc89)cc7)cc6)ccc5c4c3s2)cc1 ; c1ccc(-c2cccc(N(c3ccc(-c4ccc5c(c4)oc4ccc6nc(-c7ccccc7)sc6c45)cc3)c3ccc(-c4cc5ccccc5c5ccccc45)cc3)c2)cc1]

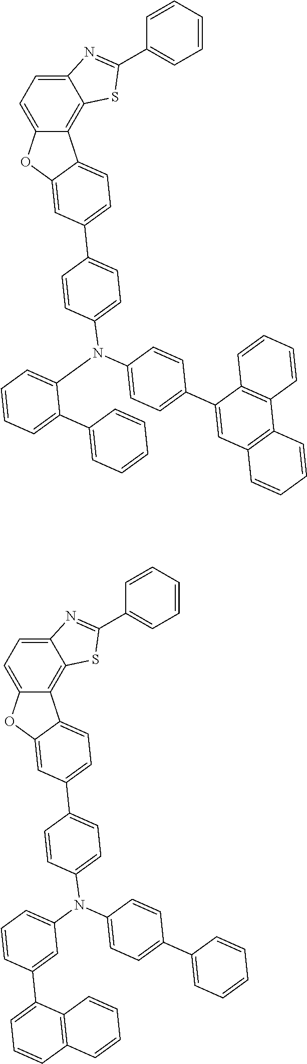 [SMILES: c1ccc(-c2nc3ccc4oc5cc(-c6ccc(N(c7ccc(-c8cc9ccccc9c9ccccc89)cc7)c7ccccc7-c7ccccc7)cc6)ccc5c4c3s2)cc1 ; c1ccc(-c2ccc(N(c3ccc(-c4ccc5c(c4)oc4ccc6nc(-c7ccccc7)sc6c45)cc3)c3cccc(-c4cccc5ccccc45)c3)cc2)cc1]

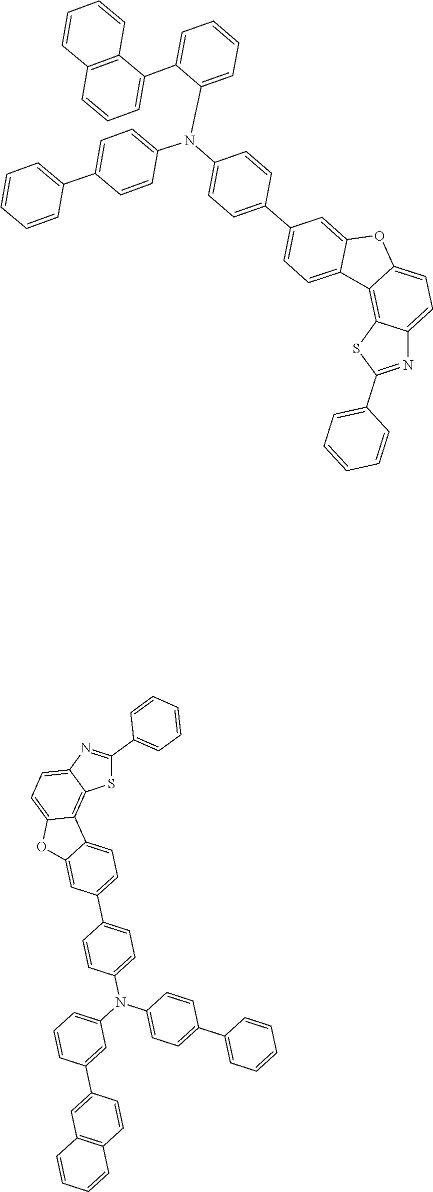 [SMILES: c1ccc(-c2ccc(N(c3ccc(-c4ccc5c(c4)oc4ccc6nc(-c7ccccc7)sc6c45)cc3)c3ccccc3-c3cccc4ccccc34)cc2)cc1 ; c1ccc(-c2ccc(N(c3ccc(-c4ccc5c(c4)oc4ccc6nc(-c7ccccc7)sc6c45)cc3)c3cccc(-c4ccc5ccccc5c4)c3)cc2)cc1]

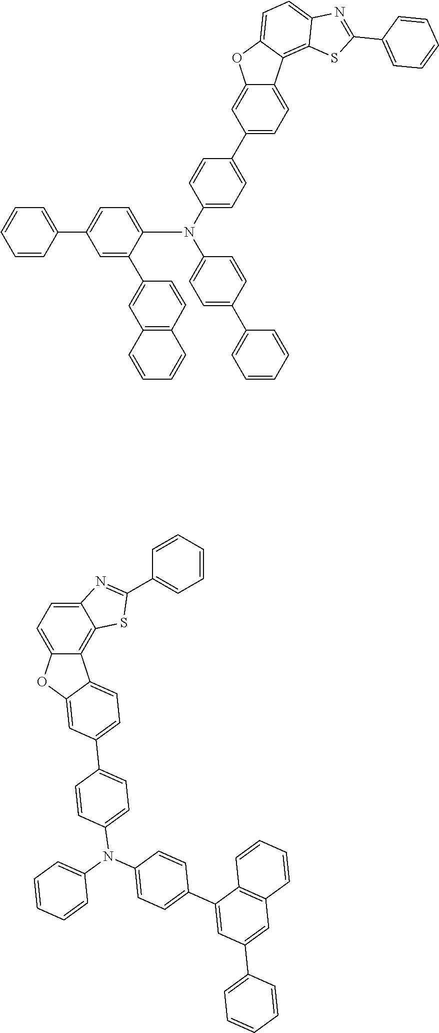 [SMILES: c1ccc(-c2ccc(N(c3ccc(-c4ccc5c(c4)oc4ccc6nc(-c7ccccc7)sc6c45)cc3)c3ccc(-c4ccccc4)cc3-c3ccc4ccccc4c3)cc2)cc1 ; c1ccc(-c2cc(-c3ccc(N(c4ccccc4)c4ccc(-c5ccc6c(c5)oc5ccc7nc(-c8ccccc8)sc7c56)cc4)cc3)c3ccccc3c2)cc1]

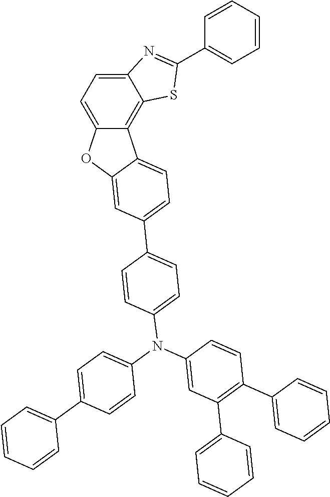 [SMILES: c1ccc(-c2ccc(N(c3ccc(-c4ccc5c(c4)oc4ccc6nc(-c7ccccc7)sc6c45)cc3)c3ccc(-c4ccccc4)c(-c4ccccc4)c3)cc2)cc1]

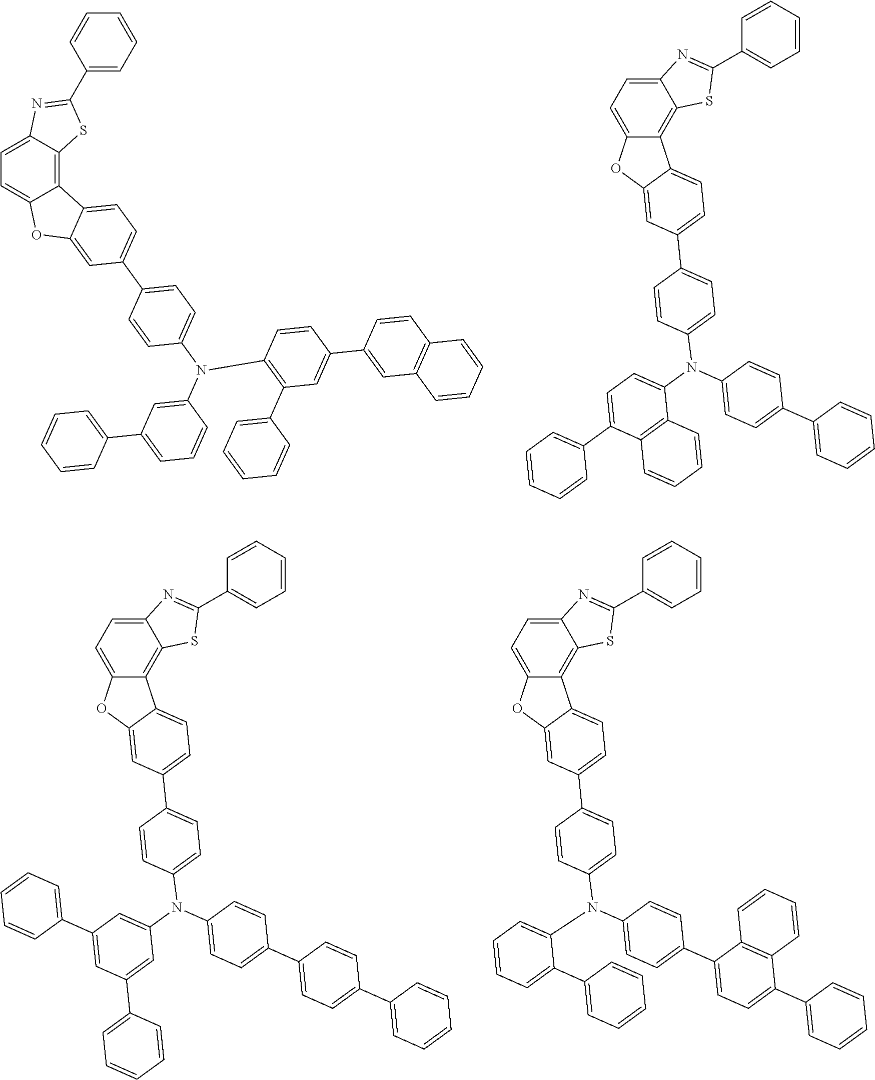 [SMILES: c1ccc(-c2cccc(N(c3ccc(-c4ccc5c(c4)oc4ccc6nc(-c7ccccc7)sc6c45)cc3)c3ccc(-c4ccc5ccccc5c4)cc3-c3ccccc3)c2)cc1 ; c1ccc(-c2ccc(N(c3ccc(-c4ccc5c(c4)oc4ccc6nc(-c7ccccc7)sc6c45)cc3)c3ccc(-c4ccccc4)c4ccccc34)cc2)cc1 ; c1ccc(-c2ccc(-c3ccc(N(c4ccc(-c5ccc6c(c5)oc5ccc7nc(-c8ccccc8)sc7c56)cc4)c4cc(-c5ccccc5)cc(-c5ccccc5)c4)cc3)cc2)cc1 ; c1ccc(-c2nc3ccc4oc5cc(-c6ccc(N(c7ccc(-c8ccc(-c9ccccc9)c9ccccc89)cc7)c7ccccc7-c7ccccc7)cc6)ccc5c4c3s2)cc1]

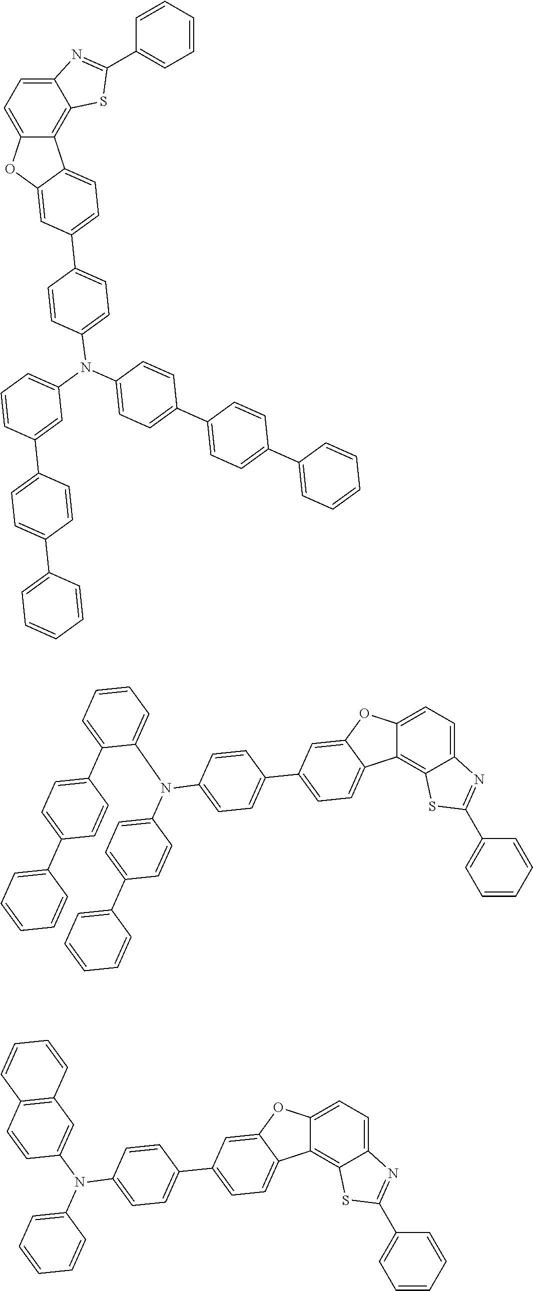 [SMILES: c1ccc(-c2ccc(-c3ccc(N(c4ccc(-c5ccc6c(c5)oc5ccc7nc(-c8ccccc8)sc7c56)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4ccc5c(c4)oc4ccc6nc(-c7ccccc7)sc6c45)cc3)cc2)cc1 ; c1ccc(-c2nc3ccc4oc5cc(-c6ccc(N(c7ccccc7)c7ccc8ccccc8c7)cc6)ccc5c4c3s2)cc1]

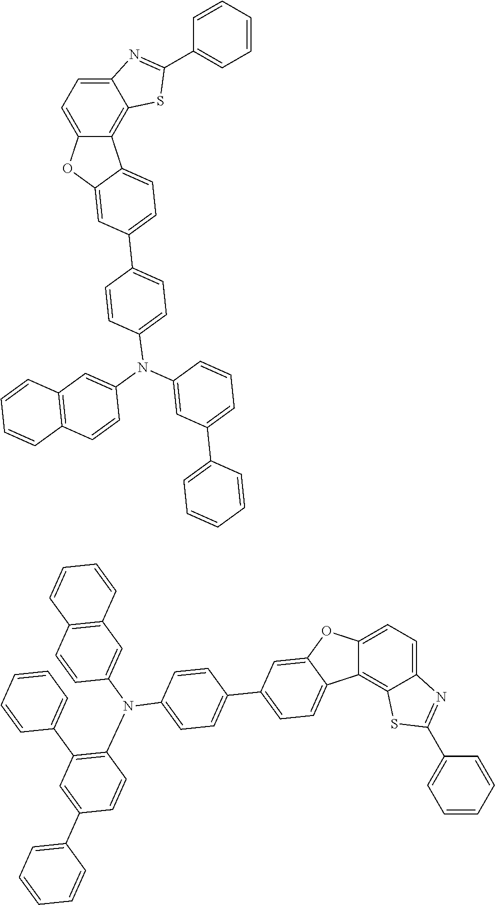 [SMILES: c1ccc(-c2cccc(N(c3ccc(-c4ccc5c(c4)oc4ccc6nc(-c7ccccc7)sc6c45)cc3)c3ccc4ccccc4c3)c2)cc1 ; c1ccc(-c2ccc(N(c3ccc(-c4ccc5c(c4)oc4ccc6nc(-c7ccccc7)sc6c45)cc3)c3ccc4ccccc4c3)c(-c3ccccc3)c2)cc1]

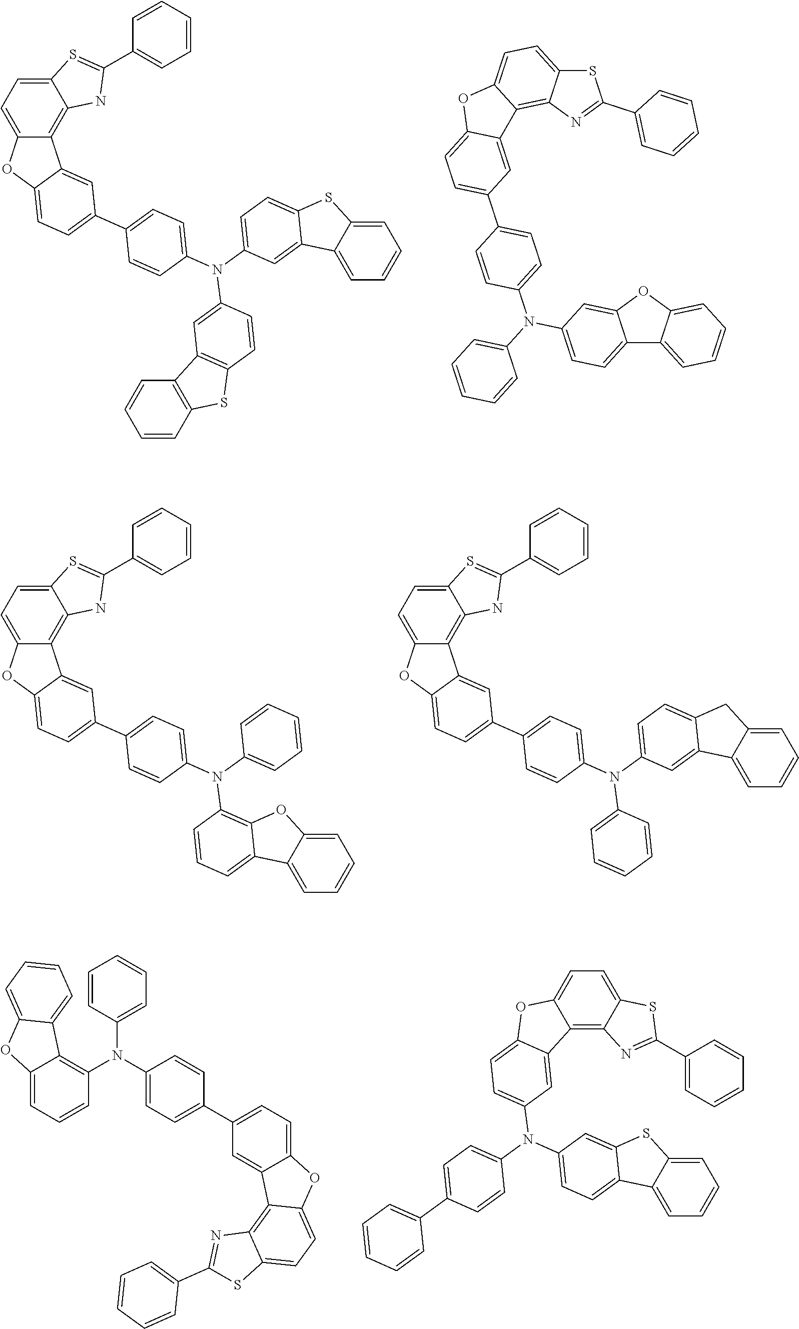

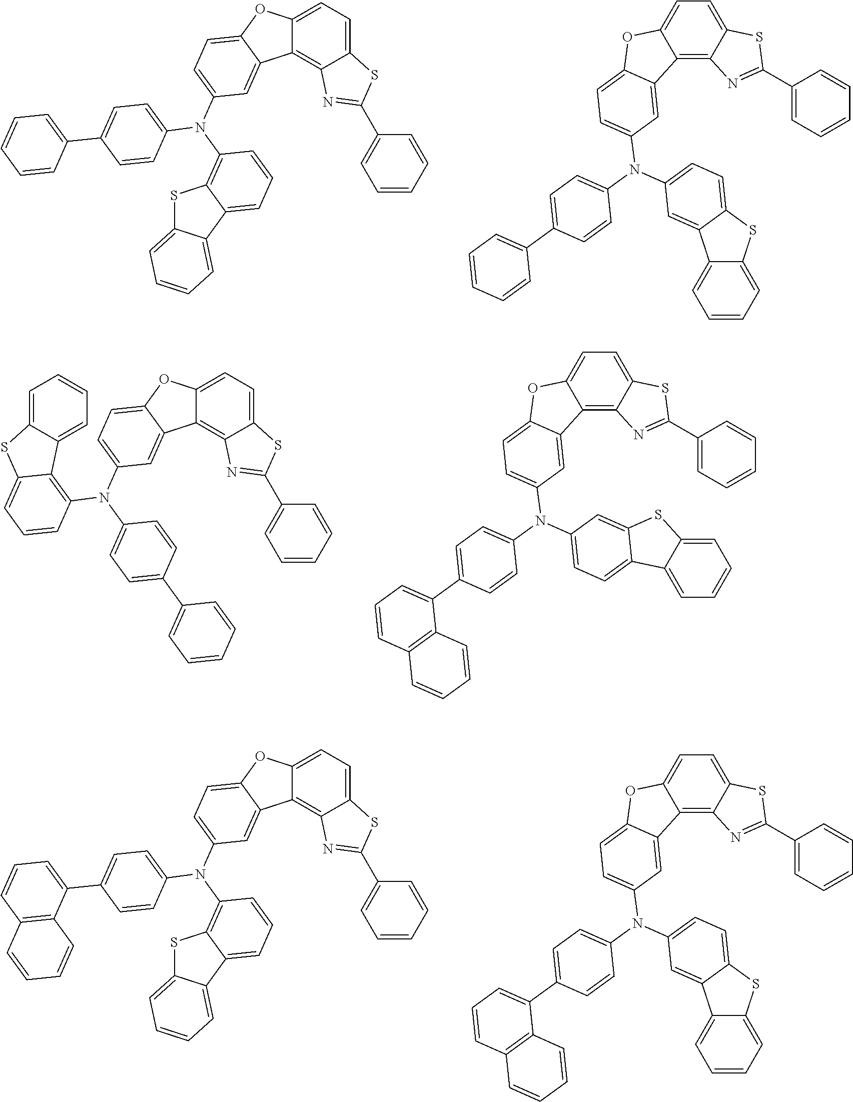

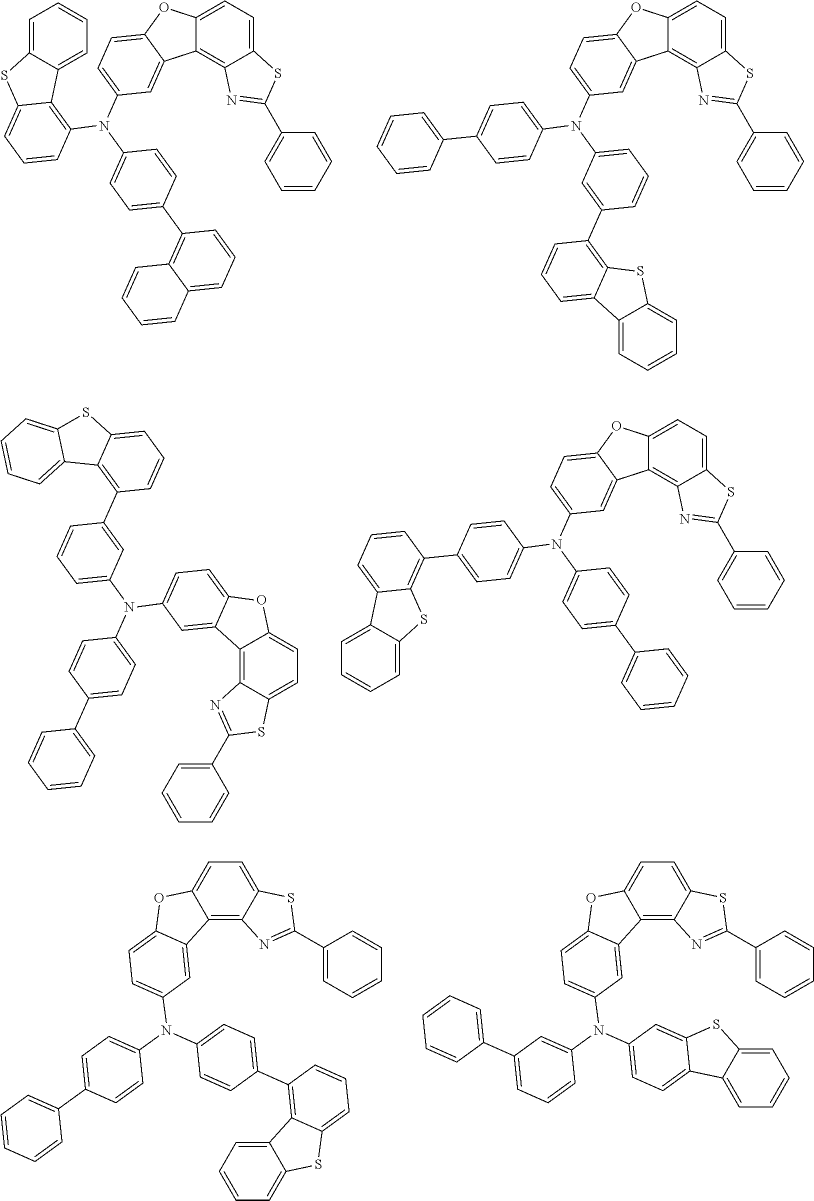

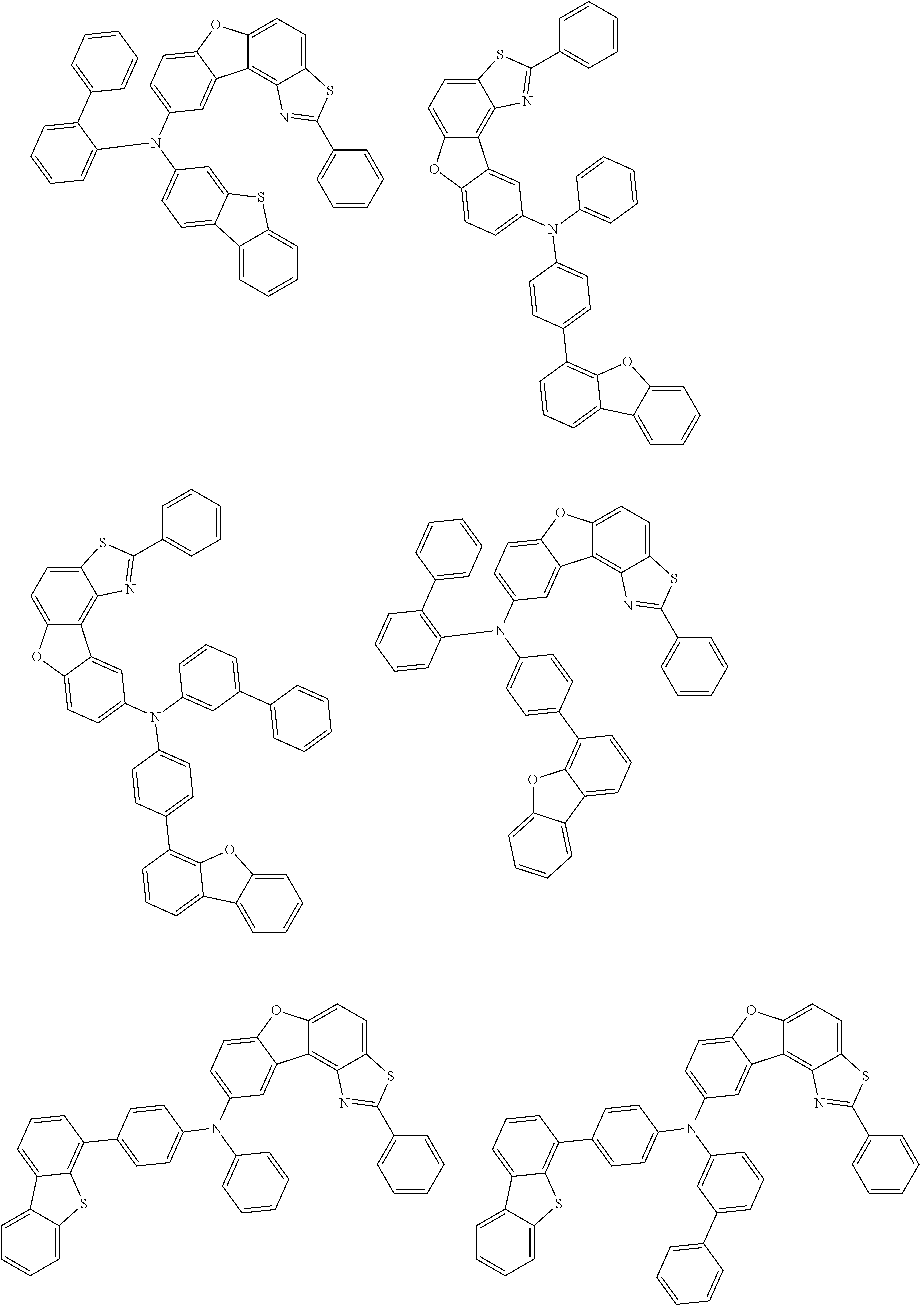

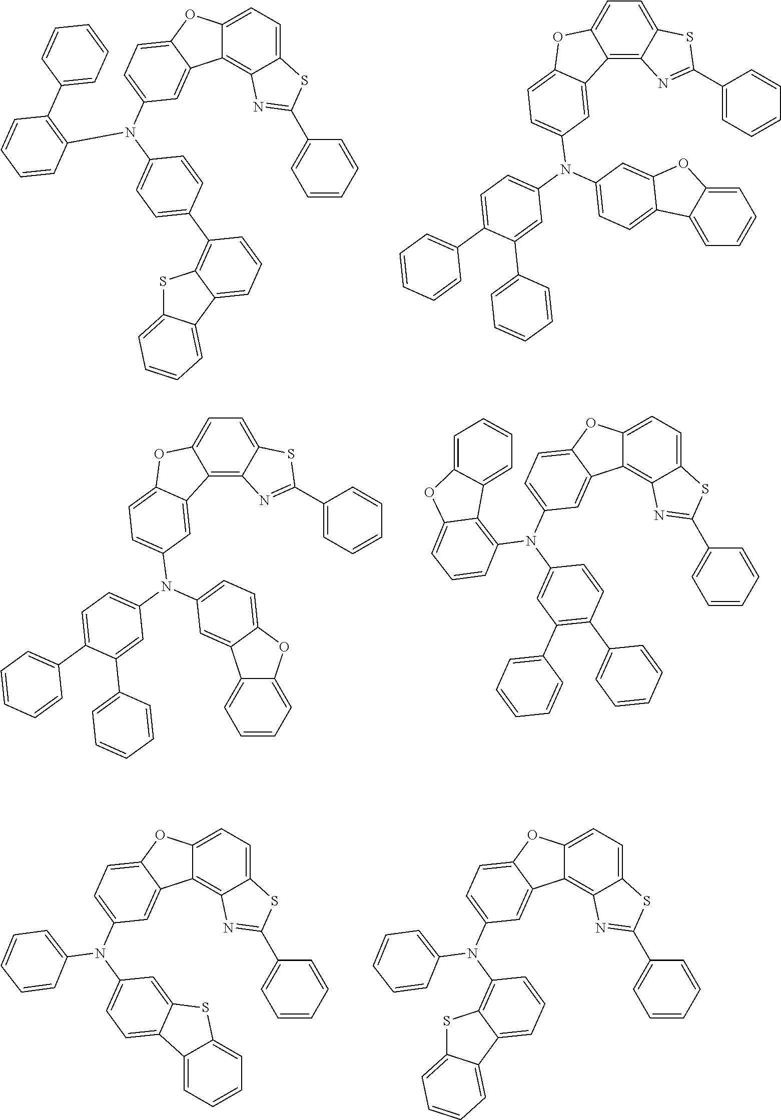

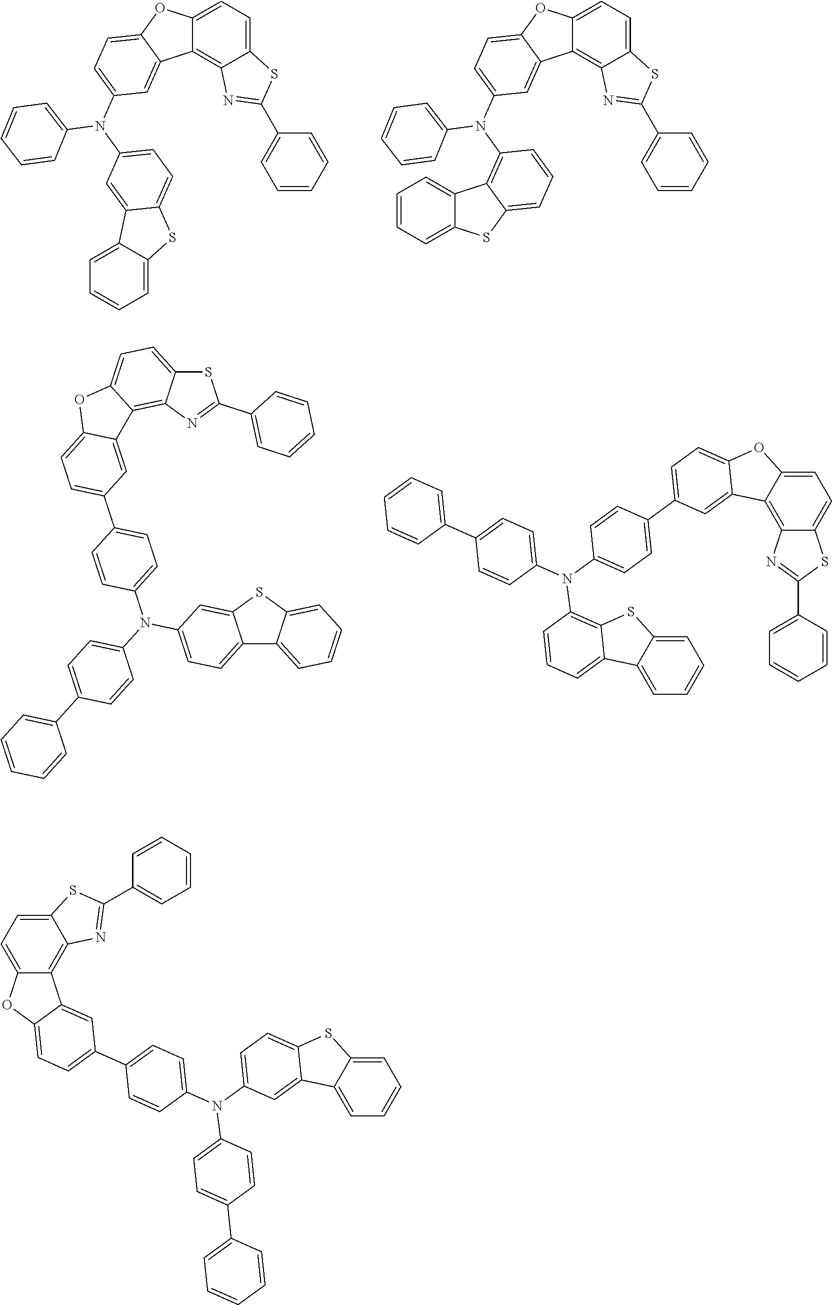

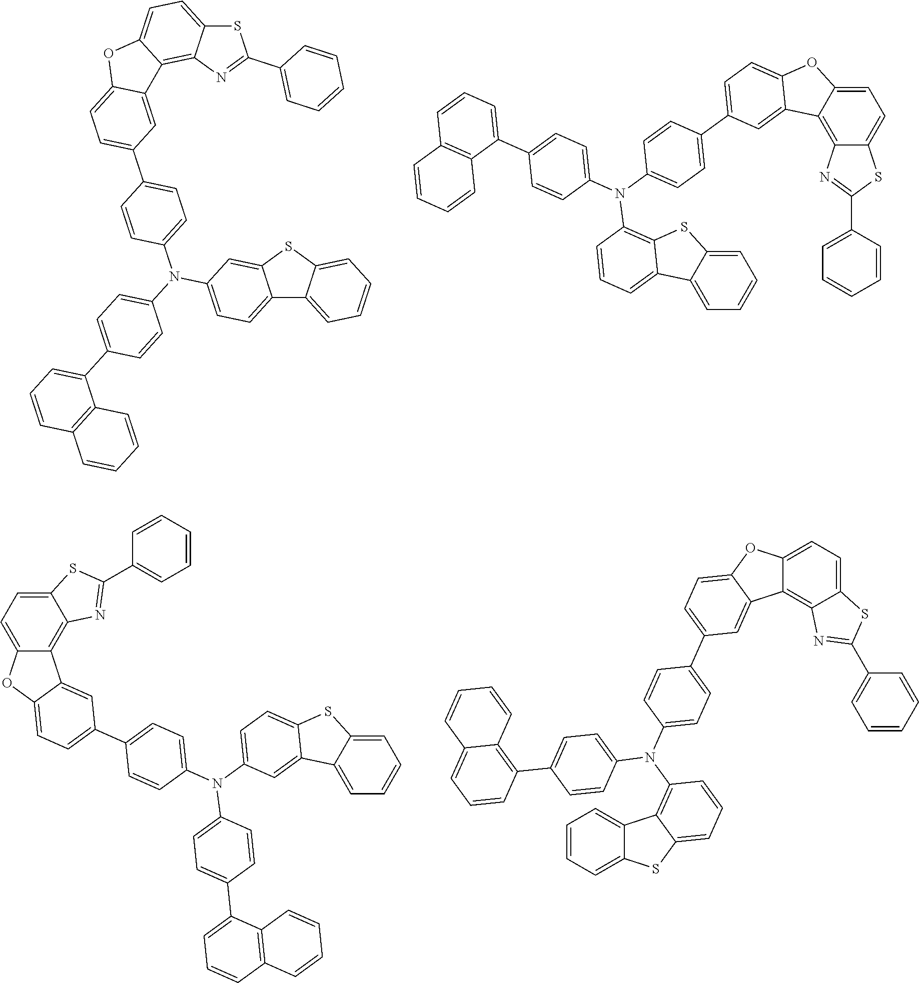 [SMILES: c1ccc(-c2nc3c(ccc4oc5ccc(-c6ccc(N(c7ccc(-c8cccc9ccccc89)cc7)c7ccc8c(c7)sc7ccccc78)cc6)cc5c43)s2)cc1 ; c1ccc(-c2nc3c(ccc4oc5ccc(-c6ccc(N(c7ccc(-c8cccc9ccccc89)cc7)c7cccc8c7sc7ccccc78)cc6)cc5c43)s2)cc1 ; c1ccc(-c2nc3c(ccc4oc5ccc(-c6ccc(N(c7ccc(-c8cccc9ccccc89)cc7)c7ccc8sc9ccccc9c8c7)cc6)cc5c43)s2)cc1 ; c1ccc(-c2nc3c(ccc4oc5ccc(-c6ccc(N(c7ccc(-c8cccc9ccccc89)cc7)c7cccc8sc9ccccc9c78)cc6)cc5c43)s2)cc1]

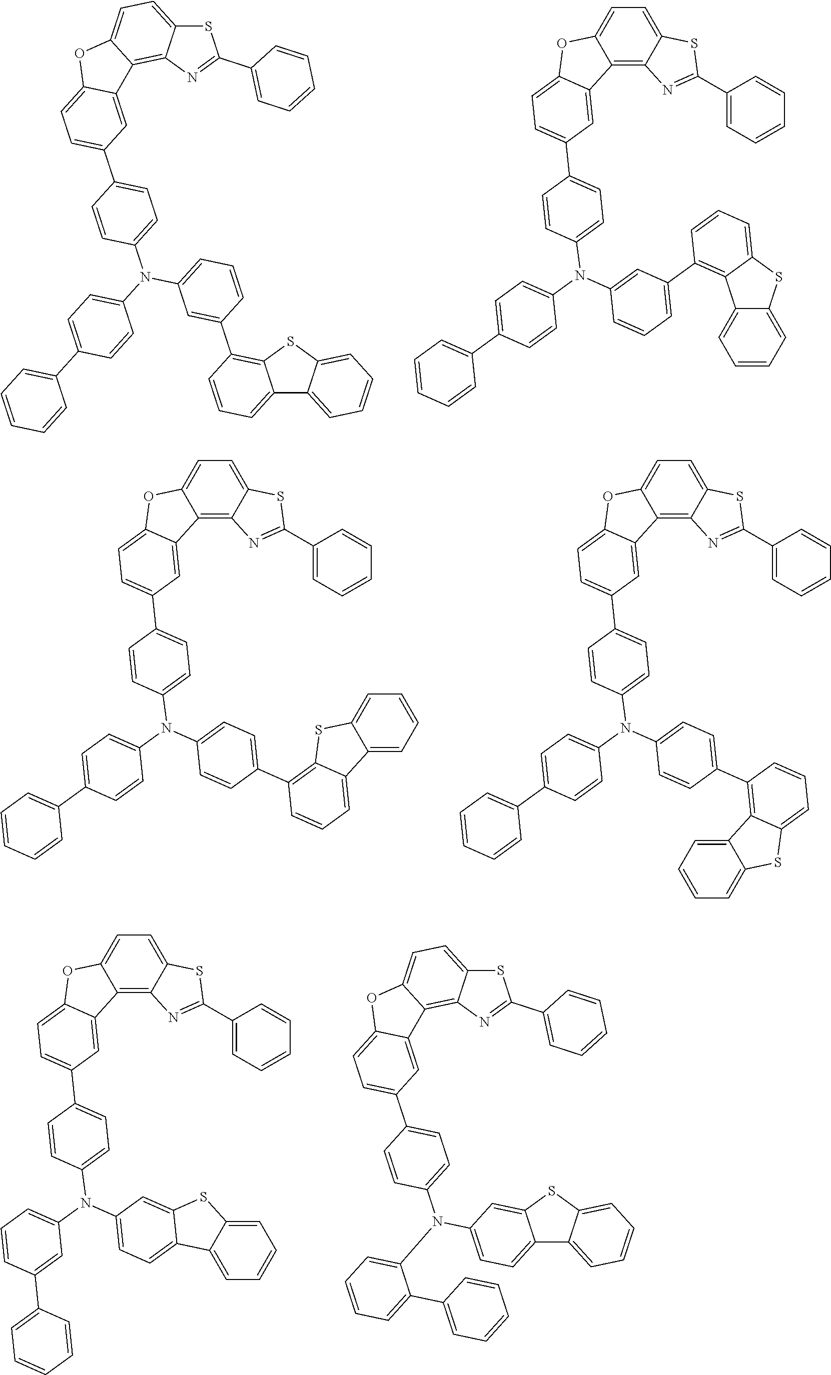

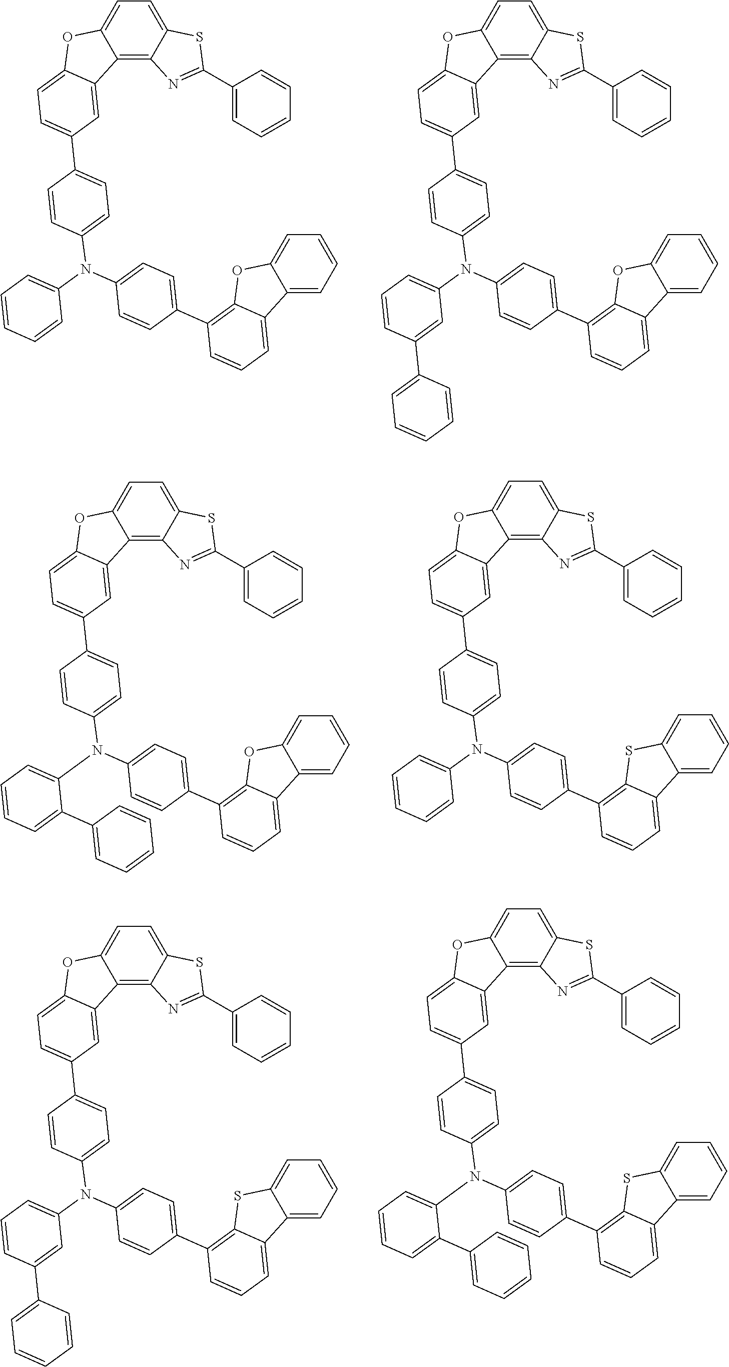

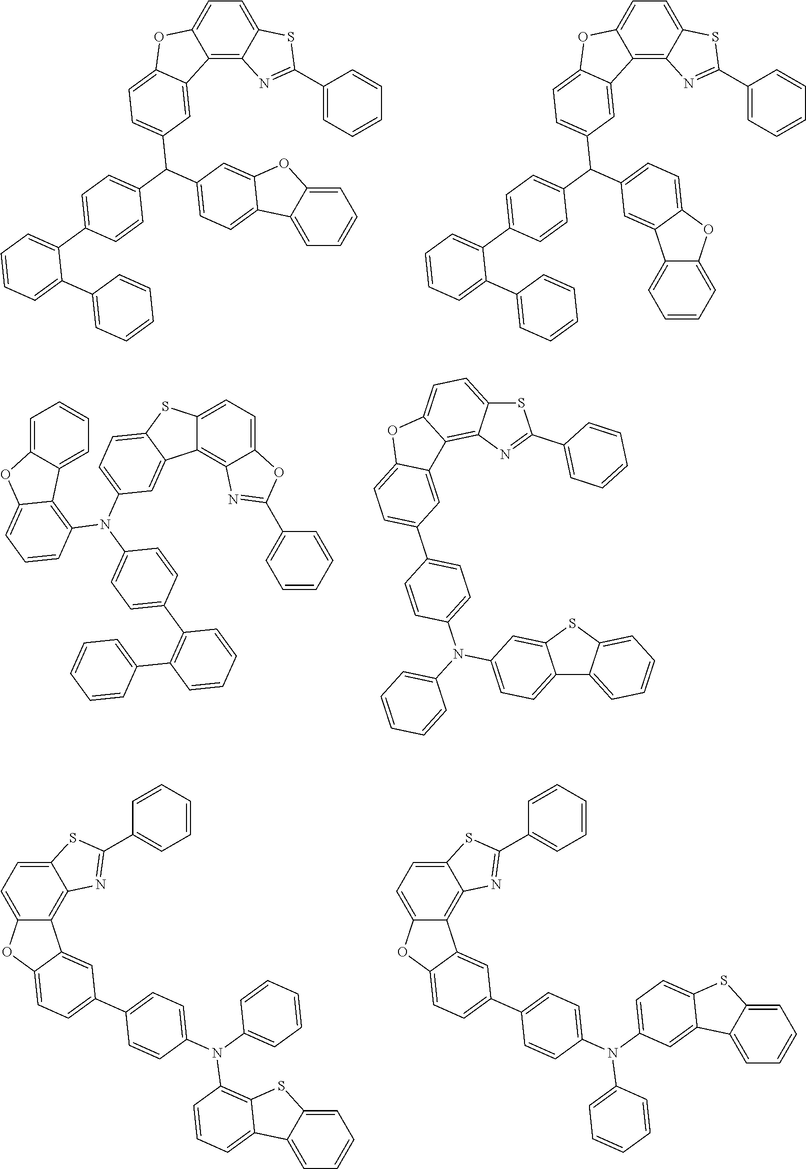

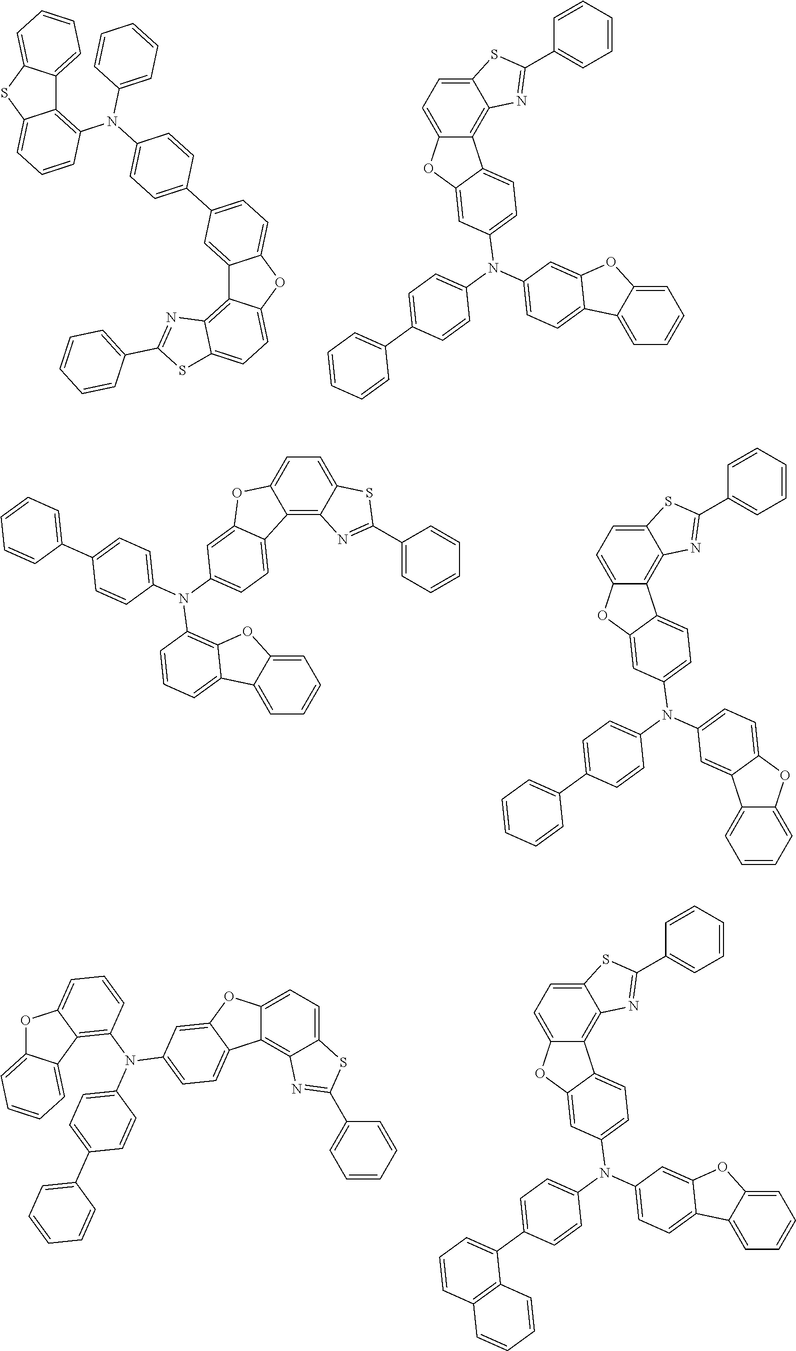

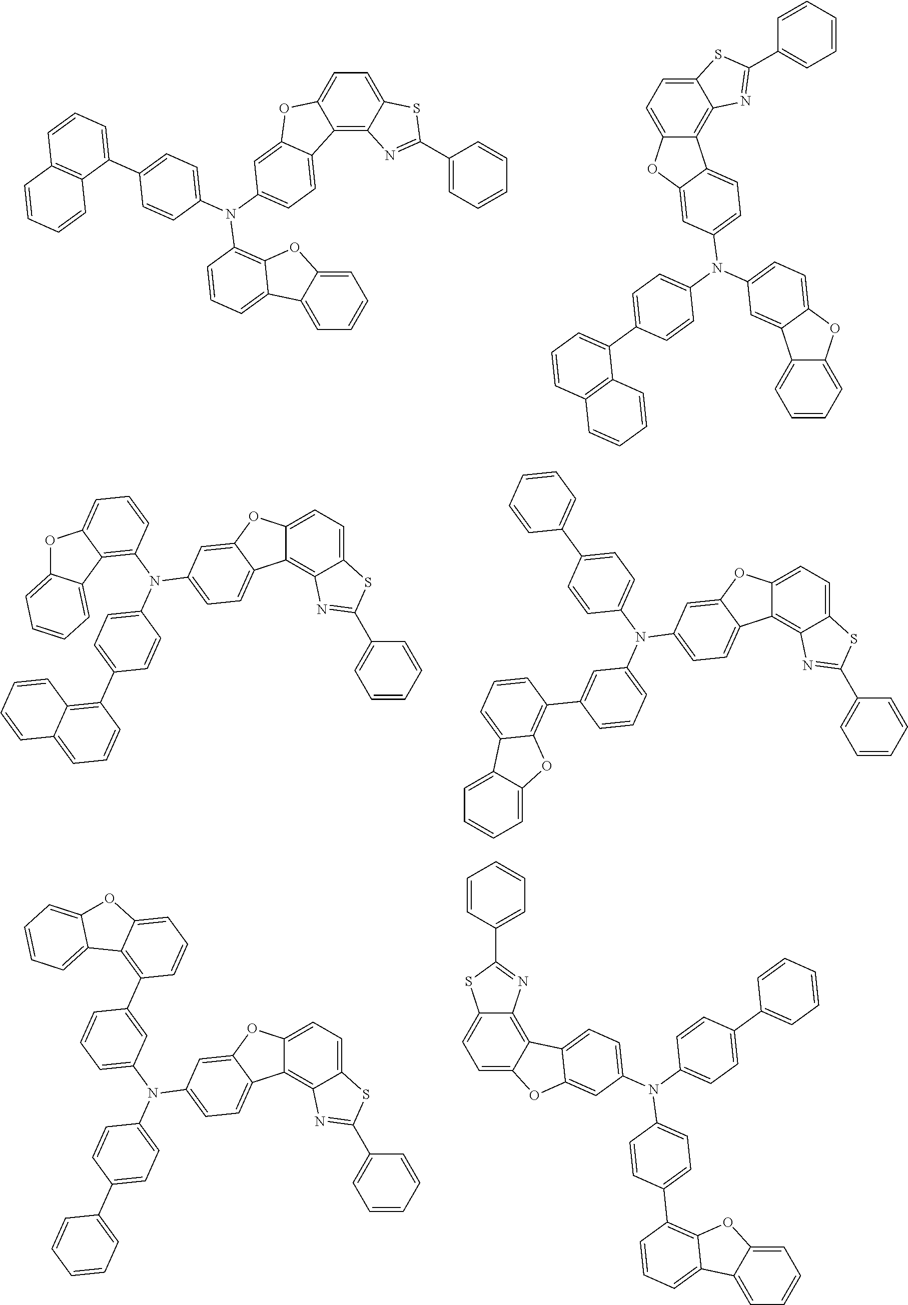

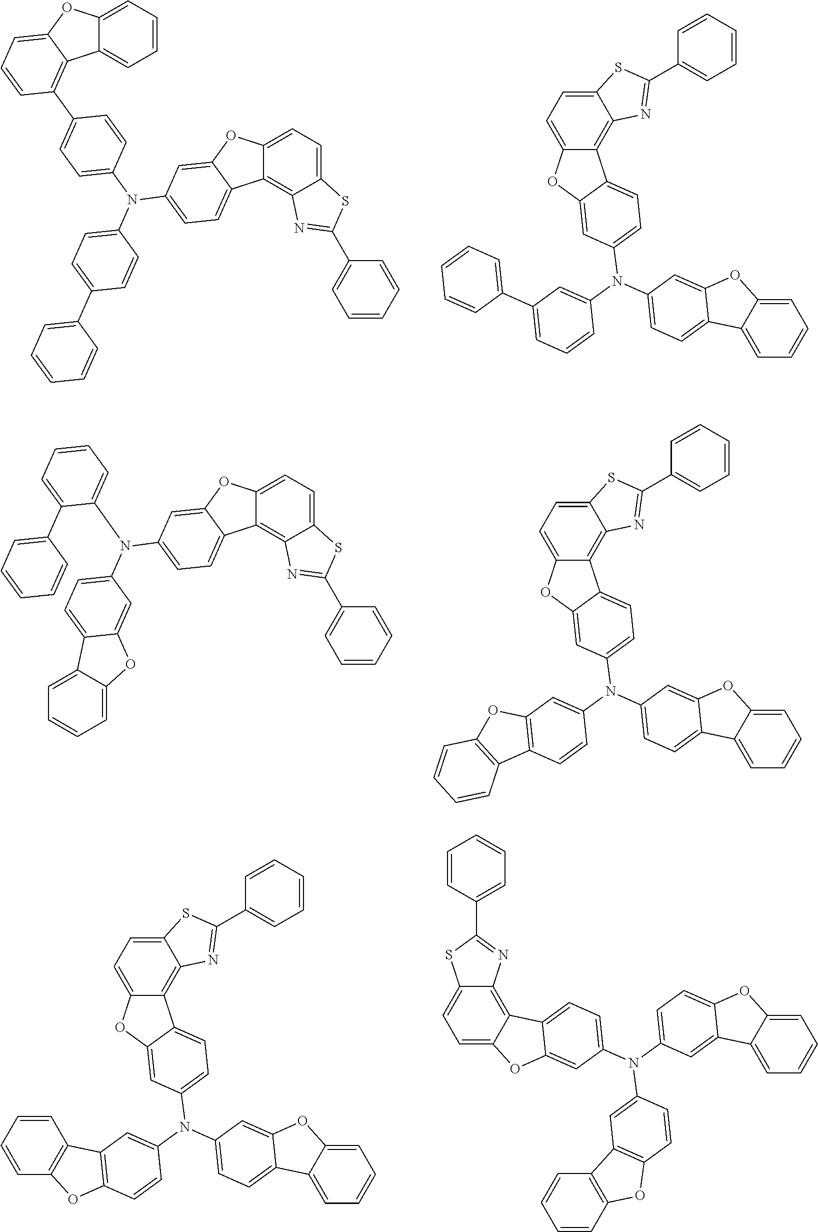

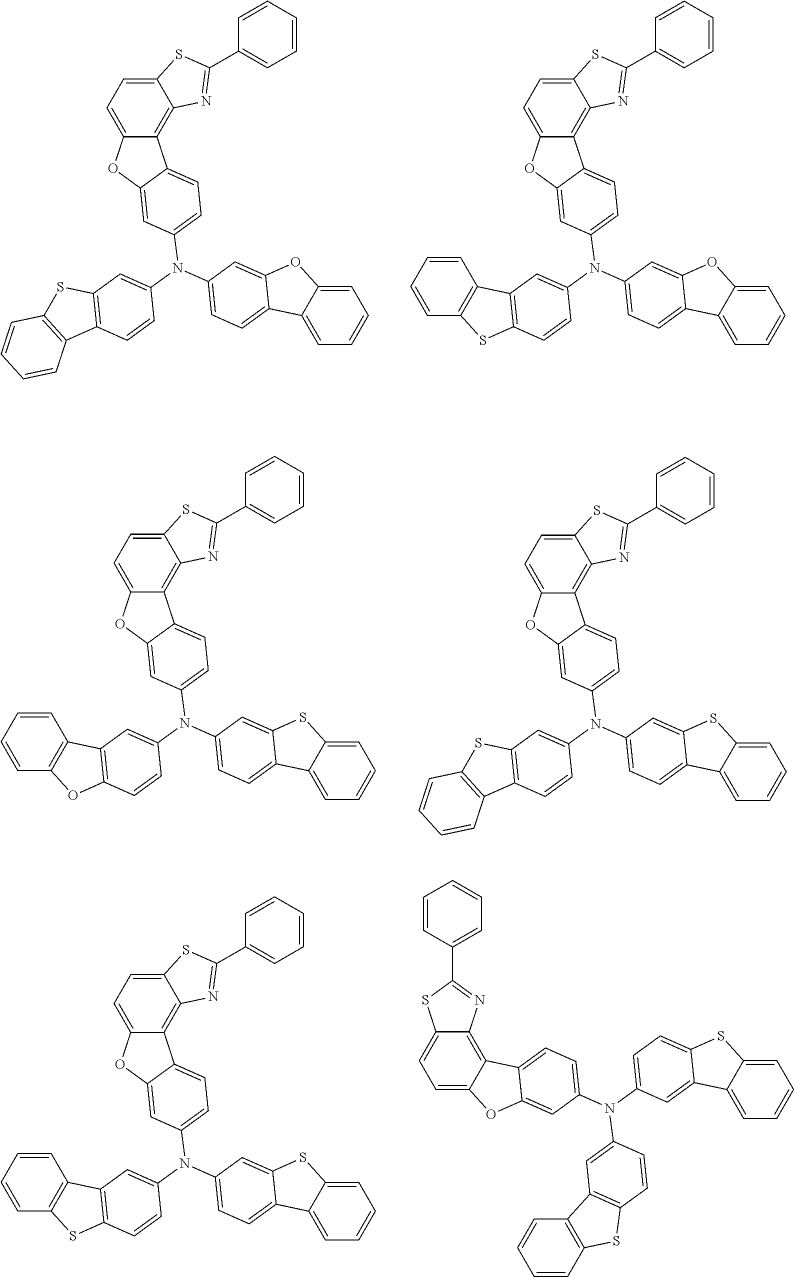

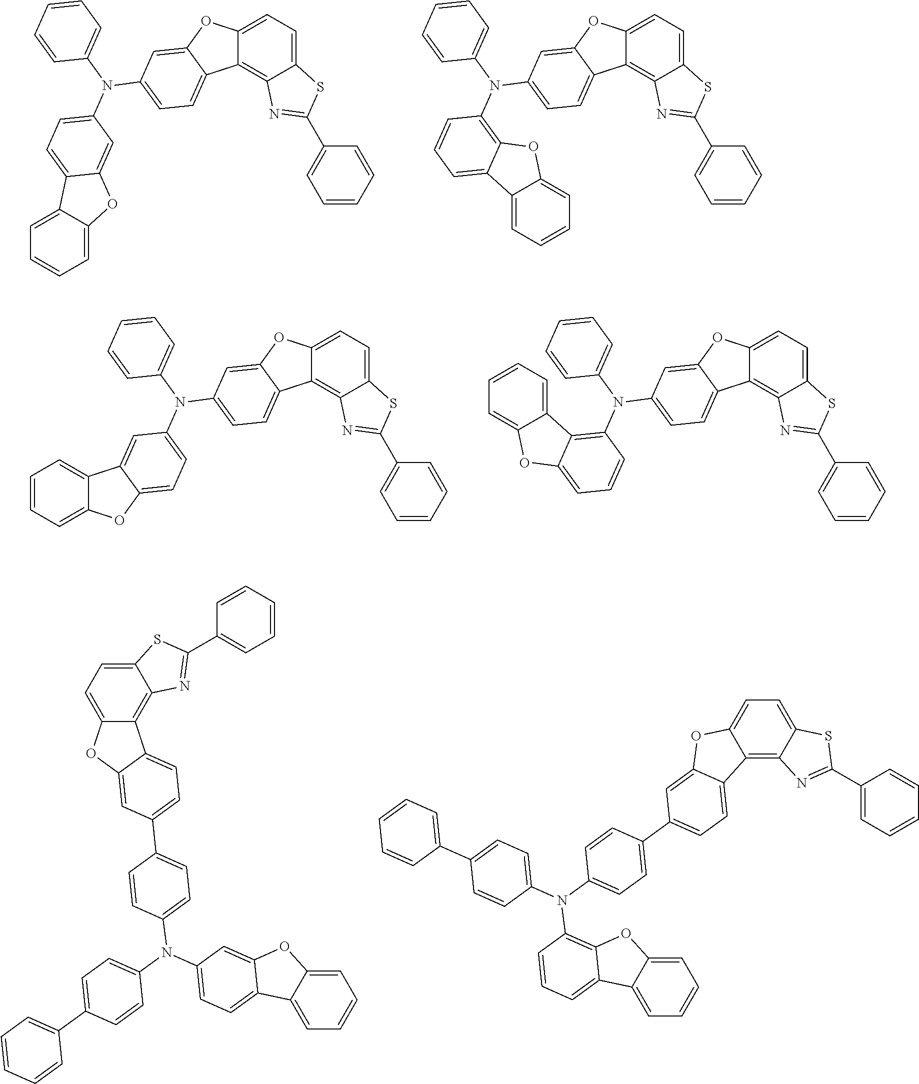 [SMILES: c1ccc(-c2nc3c(ccc4oc5cc(N(c6ccccc6)c6ccc7c(c6)oc6ccccc67)ccc5c43)s2)cc1 ; c1ccc(-c2nc3c(ccc4oc5cc(N(c6ccccc6)c6cccc7c6oc6ccccc67)ccc5c43)s2)cc1 ; c1ccc(-c2nc3c(ccc4oc5cc(N(c6ccccc6)c6ccc7oc8ccccc8c7c6)ccc5c43)s2)cc1 ; c1ccc(-c2nc3c(ccc4oc5cc(N(c6ccccc6)c6cccc7oc8ccccc8c67)ccc5c43)s2)cc1 ; c1ccc(-c2ccc(N(c3ccc(-c4ccc5c(c4)oc4ccc6sc(-c7ccccc7)nc6c45)cc3)c3ccc4c(c3)oc3ccccc34)cc2)cc1 ; c1ccc(-c2ccc(N(c3ccc(-c4ccc5c(c4)oc4ccc6sc(-c7ccccc7)nc6c45)cc3)c3cccc4c3oc3ccccc34)cc2)cc1]

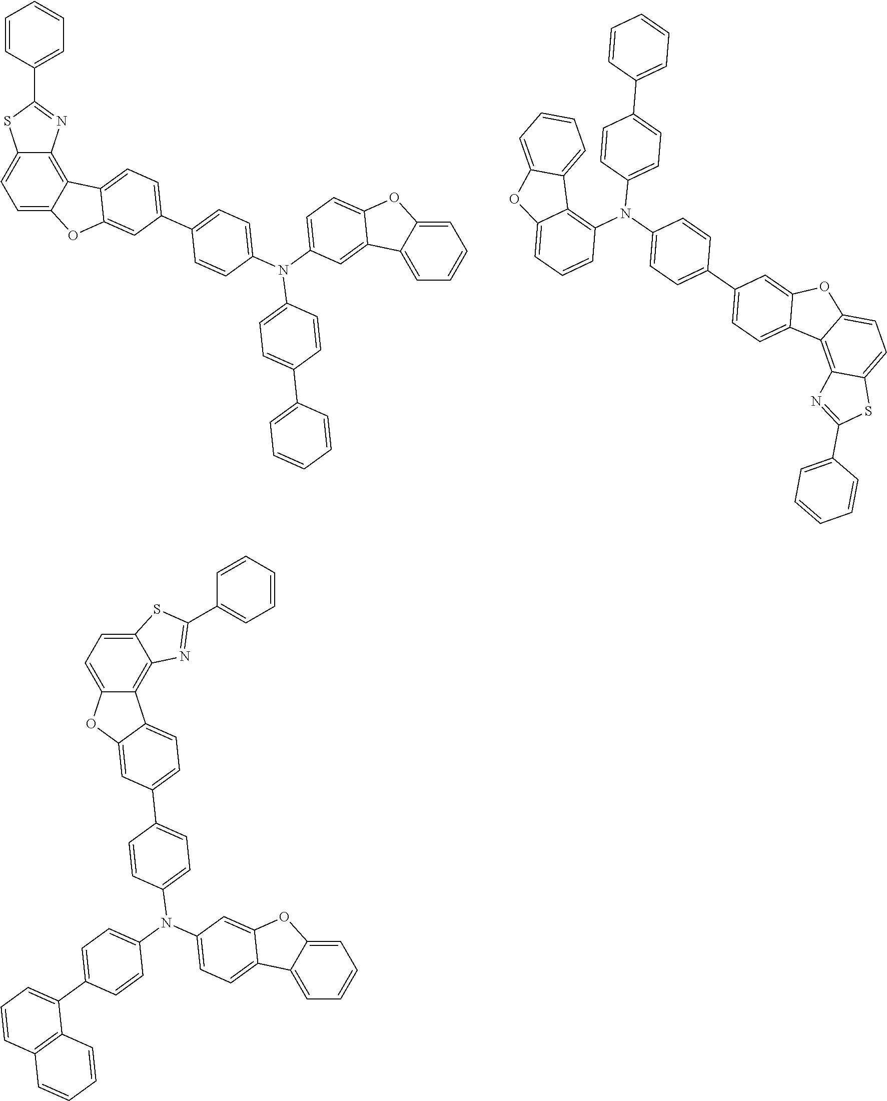 [SMILES: c1ccc(-c2ccc(N(c3ccc(-c4ccc5c(c4)oc4ccc6sc(-c7ccccc7)nc6c45)cc3)c3ccc4oc5ccccc5c4c3)cc2)cc1 ; c1ccc(-c2ccc(N(c3ccc(-c4ccc5c(c4)oc4ccc6sc(-c7ccccc7)nc6c45)cc3)c3cccc4oc5ccccc5c34)cc2)cc1 ; c1ccc(-c2nc3c(ccc4oc5cc(-c6ccc(N(c7ccc(-c8cccc9ccccc89)cc7)c7ccc8c(c7)oc7ccccc78)cc6)ccc5c43)s2)cc1]

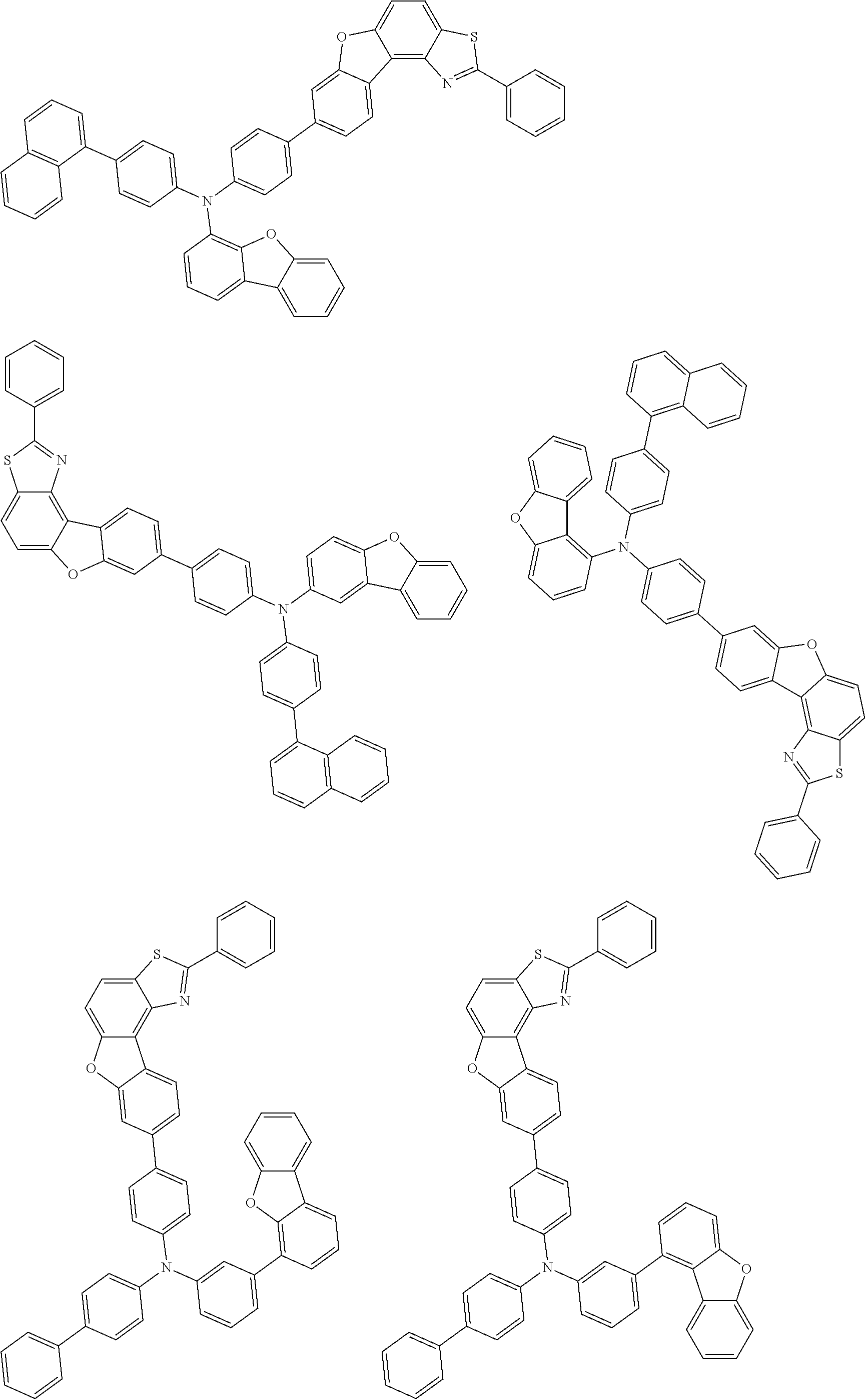

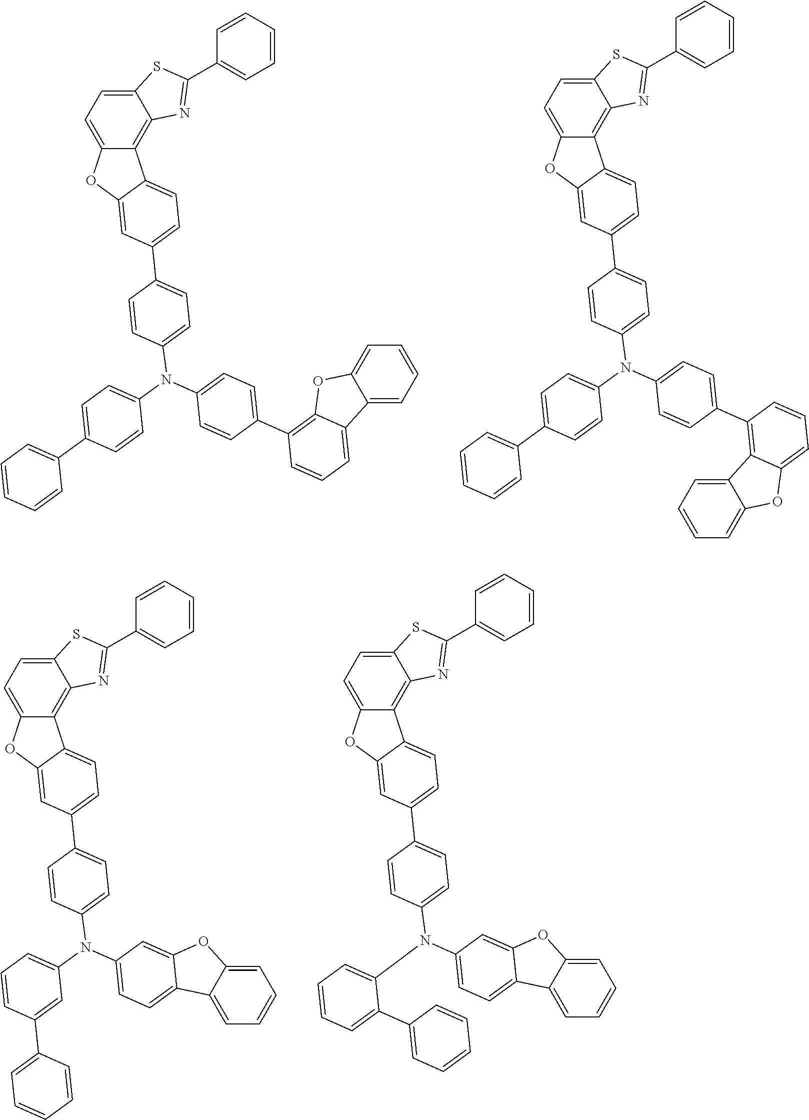

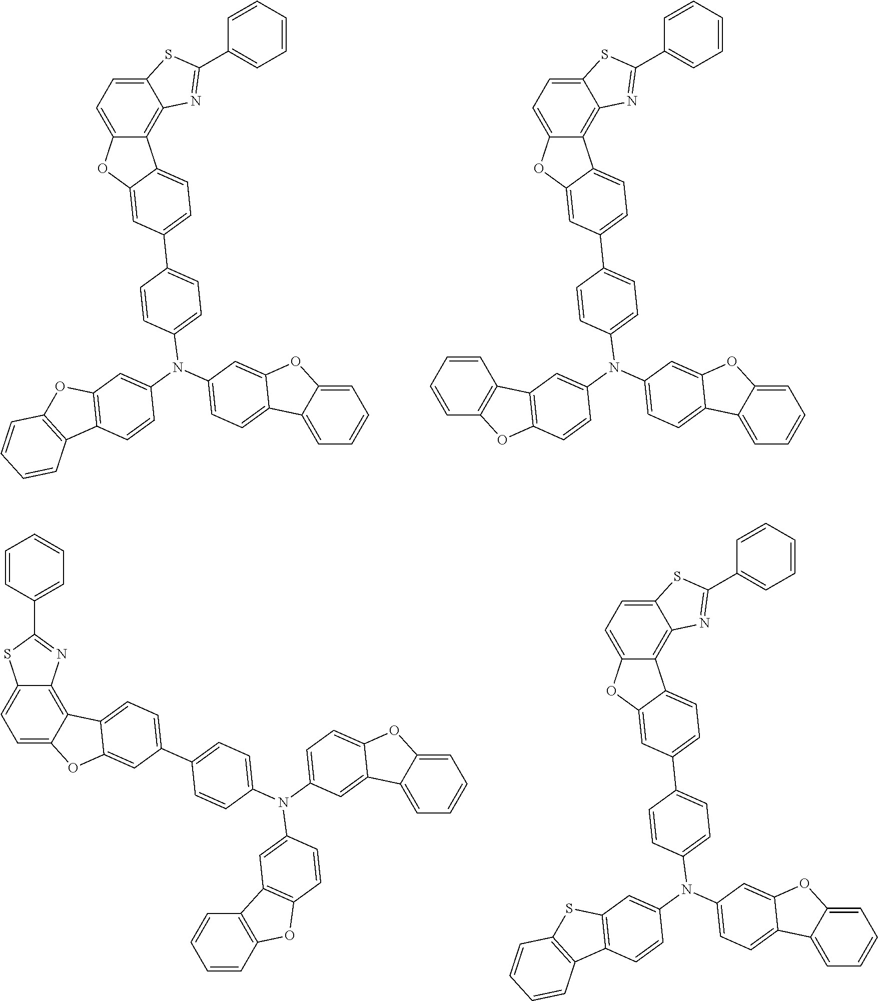 [SMILES: c1ccc(-c2nc3c(ccc4oc5cc(-c6ccc(N(c7ccc8c(c7)oc7ccccc78)c7ccc8c(c7)oc7ccccc78)cc6)ccc5c43)s2)cc1 ; c1ccc(-c2nc3c(ccc4oc5cc(-c6ccc(N(c7ccc8c(c7)oc7ccccc78)c7ccc8oc9ccccc9c8c7)cc6)ccc5c43)s2)cc1 ; c1ccc(-c2nc3c(ccc4oc5cc(-c6ccc(N(c7ccc8oc9ccccc9c8c7)c7ccc8oc9ccccc9c8c7)cc6)ccc5c43)s2)cc1 ; c1ccc(-c2nc3c(ccc4oc5cc(-c6ccc(N(c7ccc8c(c7)oc7ccccc78)c7ccc8c(c7)sc7ccccc78)cc6)ccc5c43)s2)cc1]

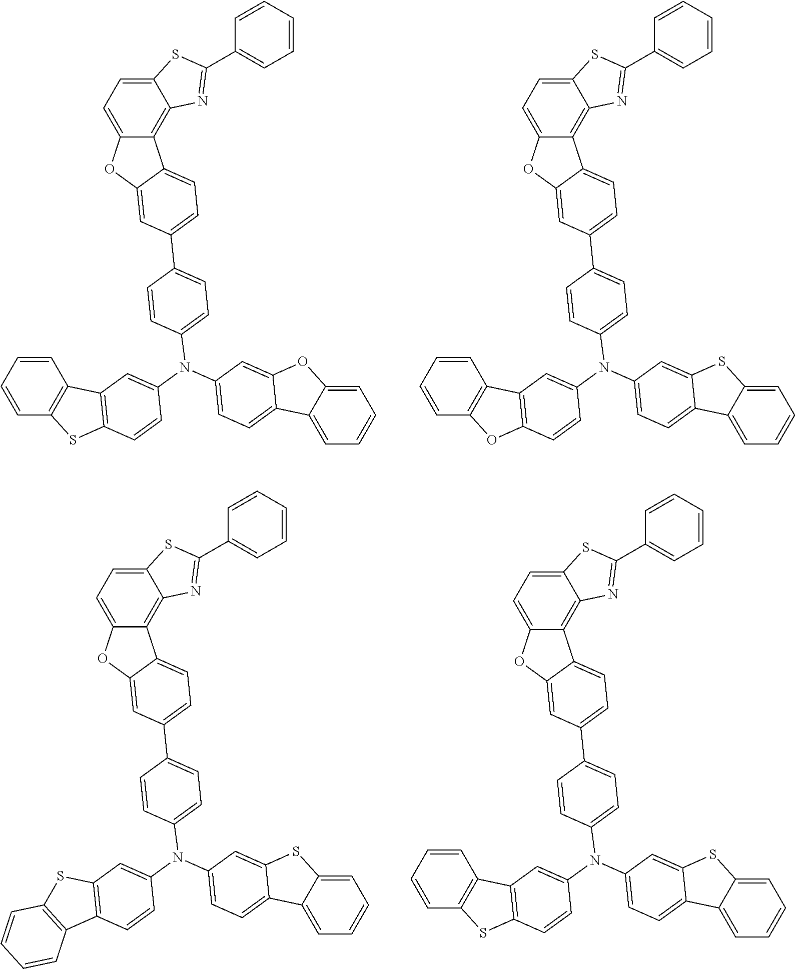 [SMILES: c1ccc(-c2nc3c(ccc4oc5cc(-c6ccc(N(c7ccc8c(c7)oc7ccccc78)c7ccc8sc9ccccc9c8c7)cc6)ccc5c43)s2)cc1 ; c1ccc(-c2nc3c(ccc4oc5cc(-c6ccc(N(c7ccc8c(c7)sc7ccccc78)c7ccc8oc9ccccc9c8c7)cc6)ccc5c43)s2)cc1 ; c1ccc(-c2nc3c(ccc4oc5cc(-c6ccc(N(c7ccc8c(c7)sc7ccccc78)c7ccc8c(c7)sc7ccccc78)cc6)ccc5c43)s2)cc1 ; c1ccc(-c2nc3c(ccc4oc5cc(-c6ccc(N(c7ccc8c(c7)sc7ccccc78)c7ccc8sc9ccccc9c8c7)cc6)ccc5c43)s2)cc1]

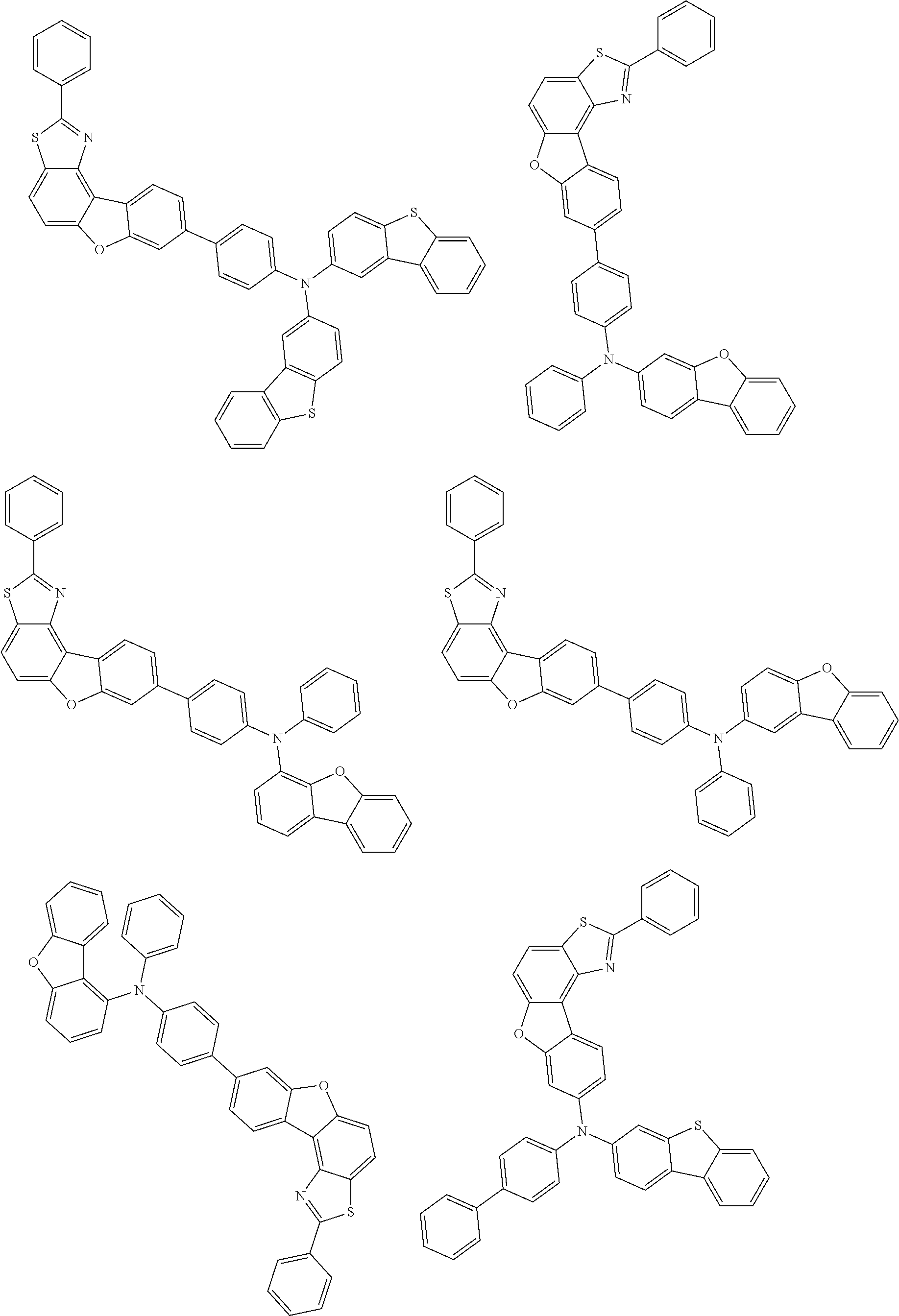

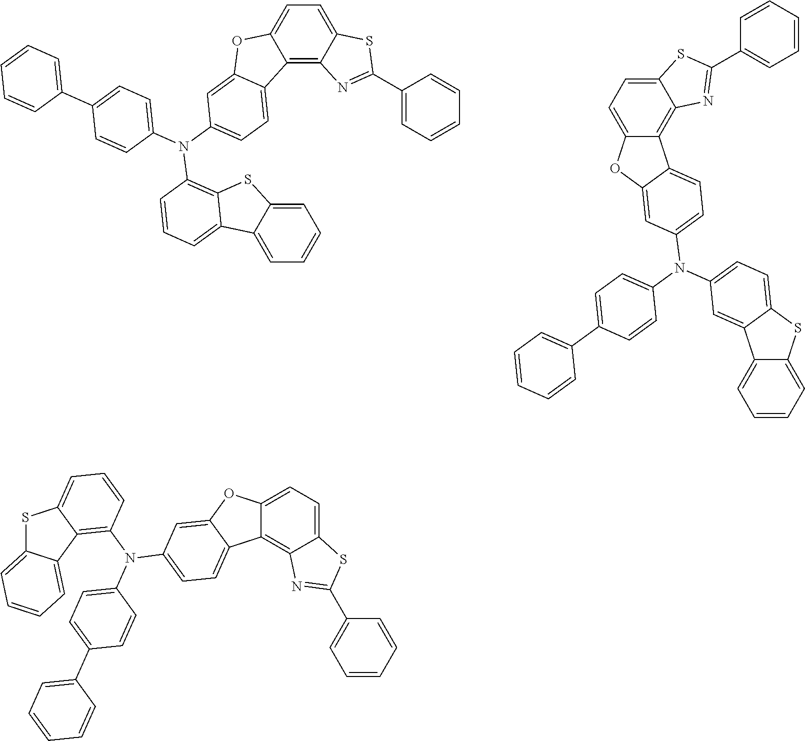 [SMILES: c1ccc(-c2ccc(N(c3ccc4c(c3)oc3ccc5sc(-c6ccccc6)nc5c34)c3cccc4c3sc3ccccc34)cc2)cc1 ; c1ccc(-c2ccc(N(c3ccc4c(c3)oc3ccc5sc(-c6ccccc6)nc5c34)c3ccc4sc5ccccc5c4c3)cc2)cc1 ; c1ccc(-c2ccc(N(c3ccc4c(c3)oc3ccc5sc(-c6ccccc6)nc5c34)c3cccc4sc5ccccc5c34)cc2)cc1]

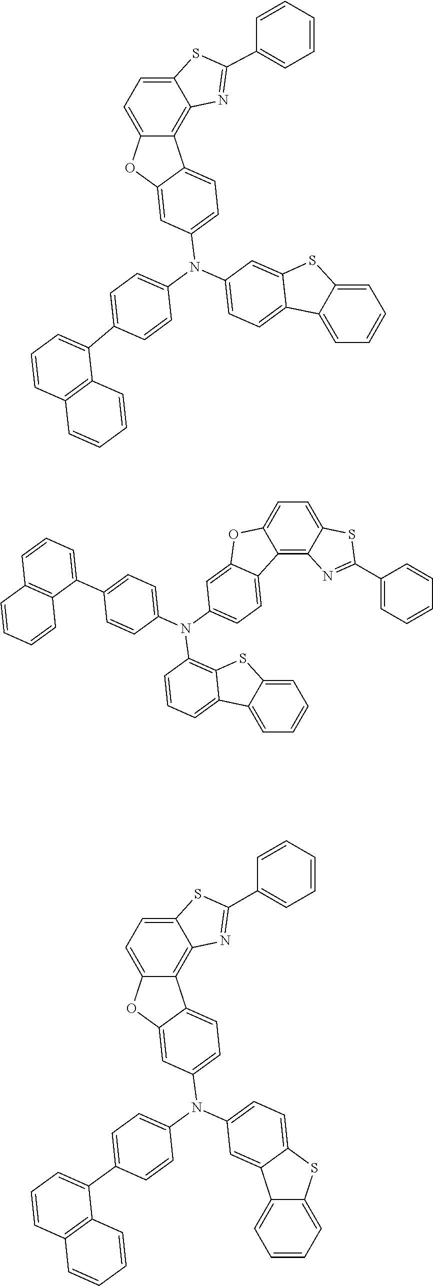 [SMILES: c1ccc(-c2nc3c(ccc4oc5cc(N(c6ccc(-c7cccc8ccccc78)cc6)c6ccc7c(c6)sc6ccccc67)ccc5c43)s2)cc1 ; c1ccc(-c2nc3c(ccc4oc5cc(N(c6ccc(-c7cccc8ccccc78)cc6)c6cccc7c6sc6ccccc67)ccc5c43)s2)cc1 ; c1ccc(-c2nc3c(ccc4oc5cc(N(c6ccc(-c7cccc8ccccc78)cc6)c6ccc7sc8ccccc8c7c6)ccc5c43)s2)cc1]

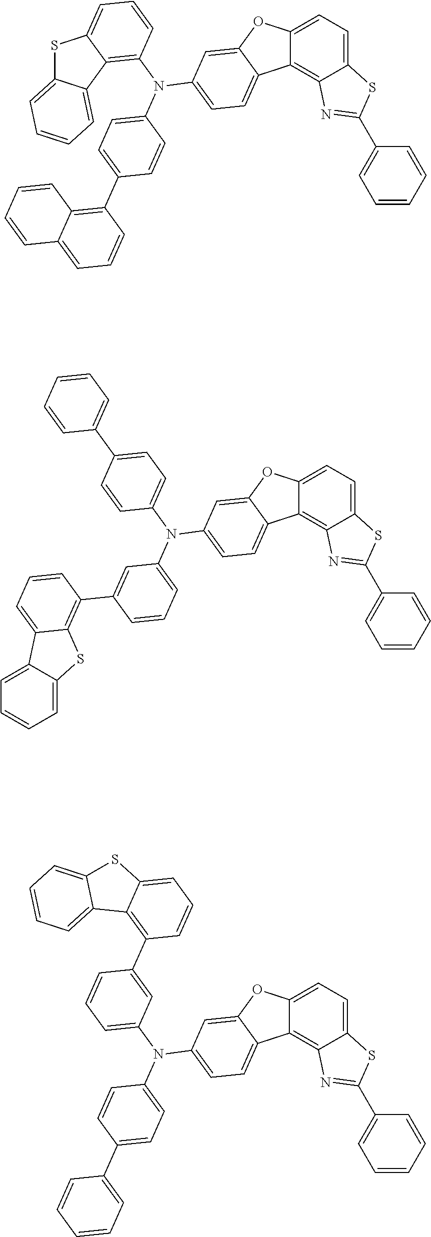 [SMILES: c1ccc(-c2nc3c(ccc4oc5cc(N(c6ccc(-c7cccc8ccccc78)cc6)c6cccc7sc8ccccc8c67)ccc5c43)s2)cc1 ; c1ccc(-c2ccc(N(c3cccc(-c4cccc5c4sc4ccccc45)c3)c3ccc4c(c3)oc3ccc5sc(-c6ccccc6)nc5c34)cc2)cc1 ; c1ccc(-c2ccc(N(c3cccc(-c4cccc5sc6ccccc6c45)c3)c3ccc4c(c3)oc3ccc5sc(-c6ccccc6)nc5c34)cc2)cc1]

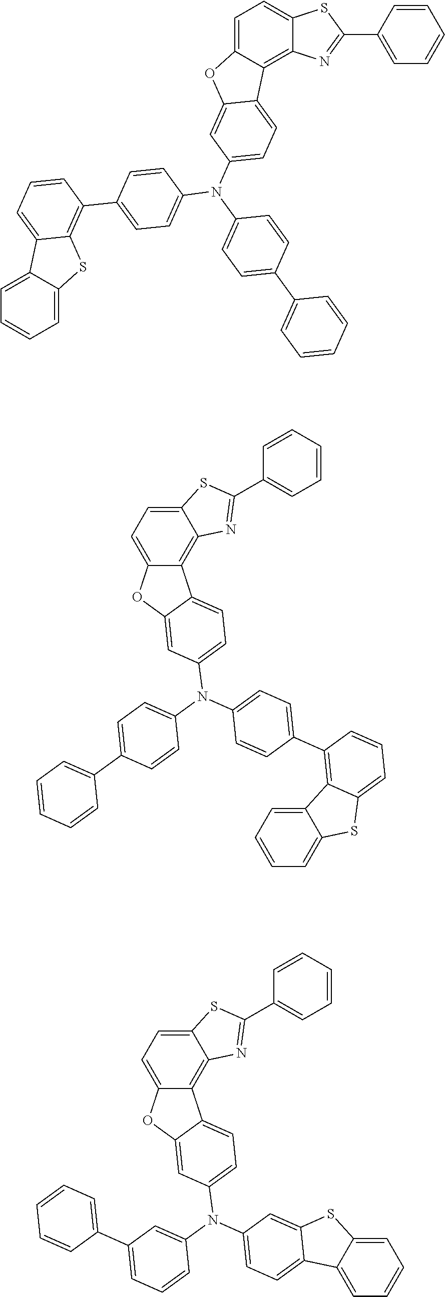 [SMILES: c1ccc(-c2ccc(N(c3ccc(-c4cccc5c4sc4ccccc45)cc3)c3ccc4c(c3)oc3ccc5sc(-c6ccccc6)nc5c34)cc2)cc1 ; c1ccc(-c2ccc(N(c3ccc(-c4cccc5sc6ccccc6c45)cc3)c3ccc4c(c3)oc3ccc5sc(-c6ccccc6)nc5c34)cc2)cc1 ; c1ccc(-c2cccc(N(c3ccc4c(c3)oc3ccc5sc(-c6ccccc6)nc5c34)c3ccc4c(c3)sc3ccccc34)c2)cc1]

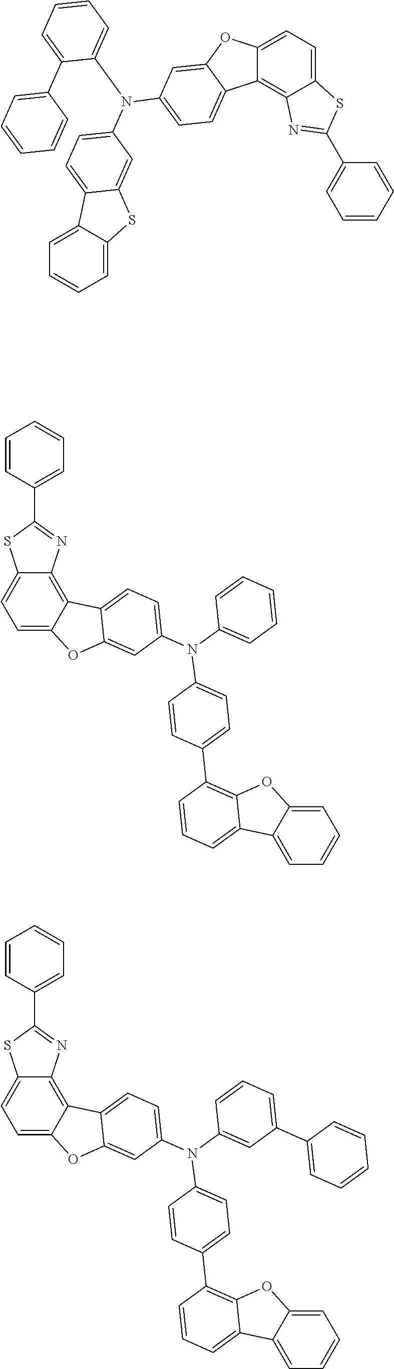 [SMILES: c1ccc(-c2nc3c(ccc4oc5cc(N(c6ccc7c(c6)sc6ccccc67)c6ccccc6-c6ccccc6)ccc5c43)s2)cc1 ; c1ccc(-c2nc3c(ccc4oc5cc(N(c6ccccc6)c6ccc(-c7cccc8c7oc7ccccc78)cc6)ccc5c43)s2)cc1 ; c1ccc(-c2cccc(N(c3ccc(-c4cccc5c4oc4ccccc45)cc3)c3ccc4c(c3)oc3ccc5sc(-c6ccccc6)nc5c34)c2)cc1]

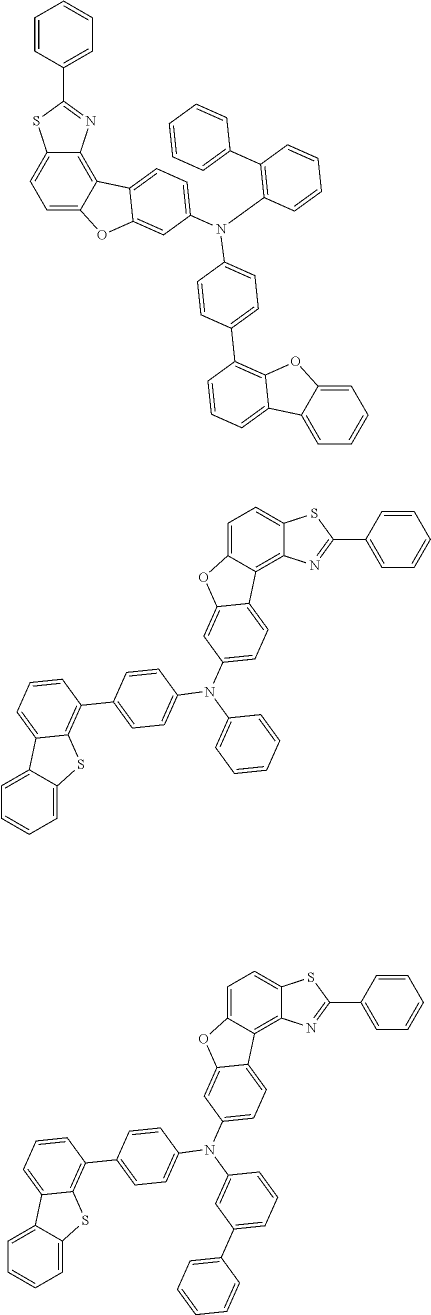 [SMILES: c1ccc(-c2nc3c(ccc4oc5cc(N(c6ccc(-c7cccc8c7oc7ccccc78)cc6)c6ccccc6-c6ccccc6)ccc5c43)s2)cc1 ; c1ccc(-c2nc3c(ccc4oc5cc(N(c6ccccc6)c6ccc(-c7cccc8c7sc7ccccc78)cc6)ccc5c43)s2)cc1 ; c1ccc(-c2cccc(N(c3ccc(-c4cccc5c4sc4ccccc45)cc3)c3ccc4c(c3)oc3ccc5sc(-c6ccccc6)nc5c34)c2)cc1]

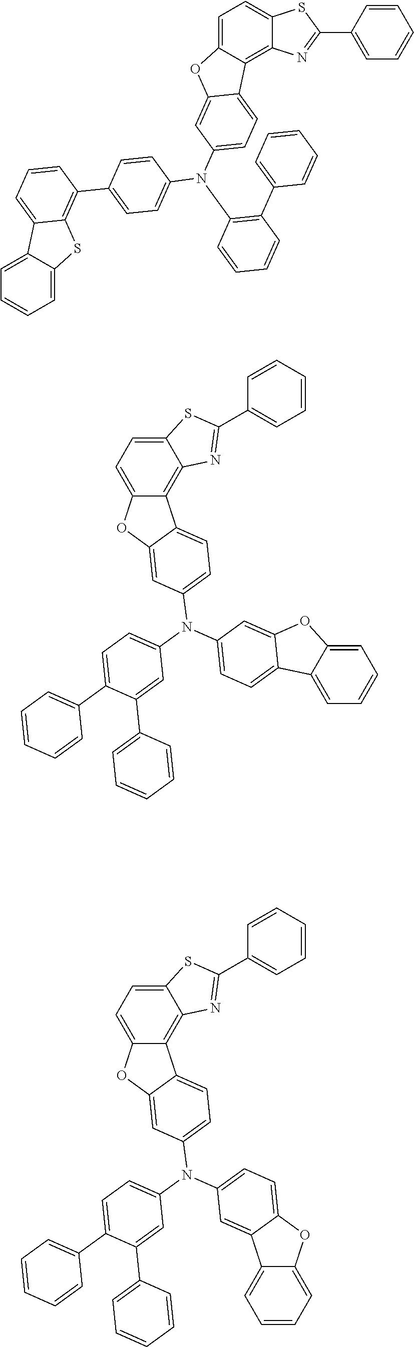 [SMILES: c1ccc(-c2nc3c(ccc4oc5cc(N(c6ccc(-c7cccc8c7sc7ccccc78)cc6)c6ccccc6-c6ccccc6)ccc5c43)s2)cc1 ; c1ccc(-c2nc3c(ccc4oc5cc(N(c6ccc(-c7ccccc7)c(-c7ccccc7)c6)c6ccc7c(c6)oc6ccccc67)ccc5c43)s2)cc1 ; c1ccc(-c2nc3c(ccc4oc5cc(N(c6ccc(-c7ccccc7)c(-c7ccccc7)c6)c6ccc7oc8ccccc8c7c6)ccc5c43)s2)cc1]

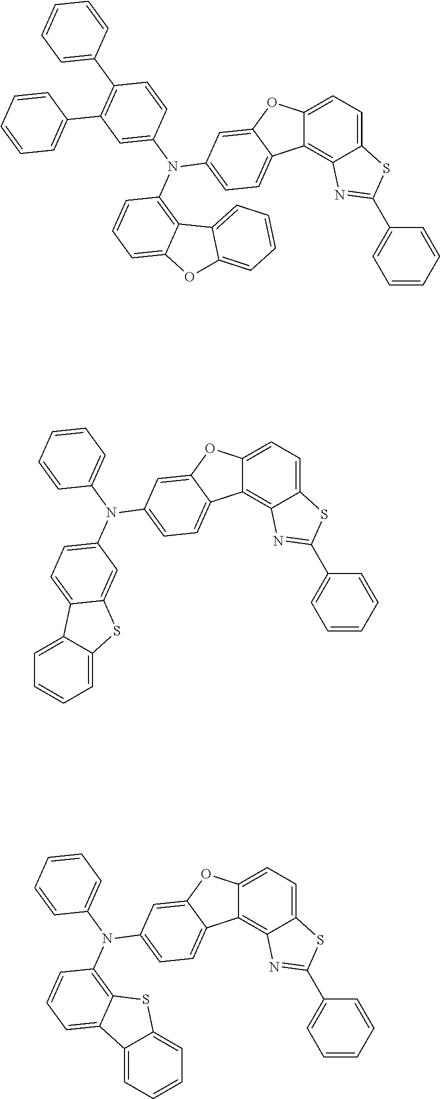 [SMILES: c1ccc(-c2nc3c(ccc4oc5cc(N(c6ccc(-c7ccccc7)c(-c7ccccc7)c6)c6cccc7oc8ccccc8c67)ccc5c43)s2)cc1 ; c1ccc(-c2nc3c(ccc4oc5cc(N(c6ccccc6)c6ccc7c(c6)sc6ccccc67)ccc5c43)s2)cc1 ; c1ccc(-c2nc3c(ccc4oc5cc(N(c6ccccc6)c6cccc7c6sc6ccccc67)ccc5c43)s2)cc1]

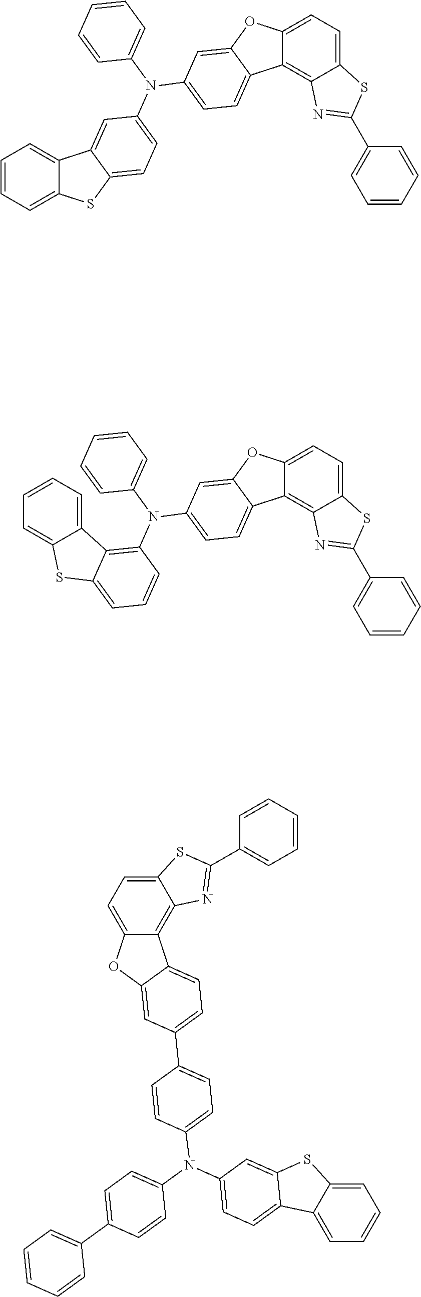 [SMILES: c1ccc(-c2nc3c(ccc4oc5cc(N(c6ccccc6)c6ccc7sc8ccccc8c7c6)ccc5c43)s2)cc1 ; c1ccc(-c2nc3c(ccc4oc5cc(N(c6ccccc6)c6cccc7sc8ccccc8c67)ccc5c43)s2)cc1 ; c1ccc(-c2ccc(N(c3ccc(-c4ccc5c(c4)oc4ccc6sc(-c7ccccc7)nc6c45)cc3)c3ccc4c(c3)sc3ccccc34)cc2)cc1]

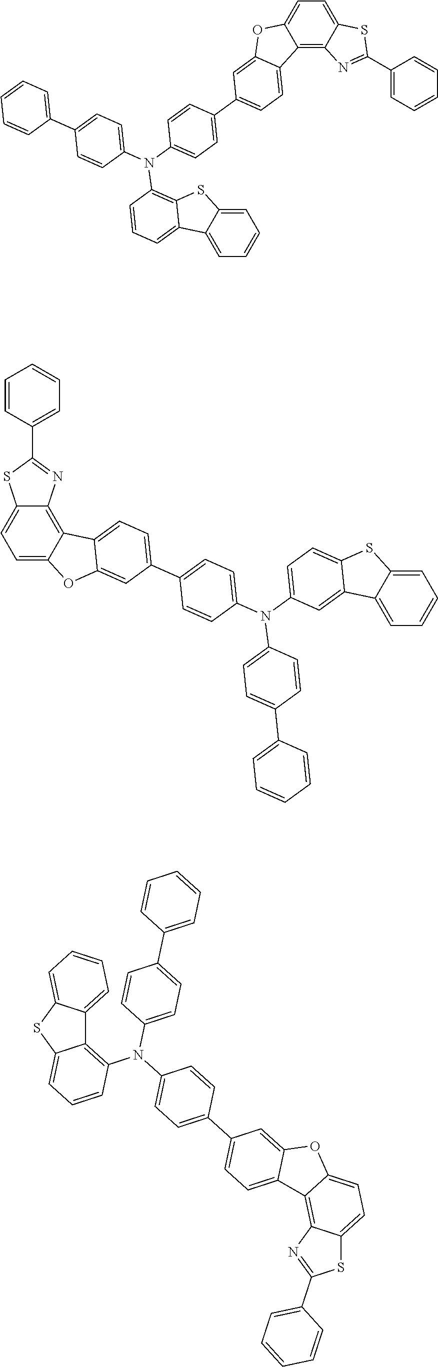 [SMILES: c1ccc(-c2ccc(N(c3ccc(-c4ccc5c(c4)oc4ccc6sc(-c7ccccc7)nc6c45)cc3)c3cccc4c3sc3ccccc34)cc2)cc1 ; c1ccc(-c2ccc(N(c3ccc(-c4ccc5c(c4)oc4ccc6sc(-c7ccccc7)nc6c45)cc3)c3ccc4sc5ccccc5c4c3)cc2)cc1 ; c1ccc(-c2ccc(N(c3ccc(-c4ccc5c(c4)oc4ccc6sc(-c7ccccc7)nc6c45)cc3)c3cccc4sc5ccccc5c34)cc2)cc1]

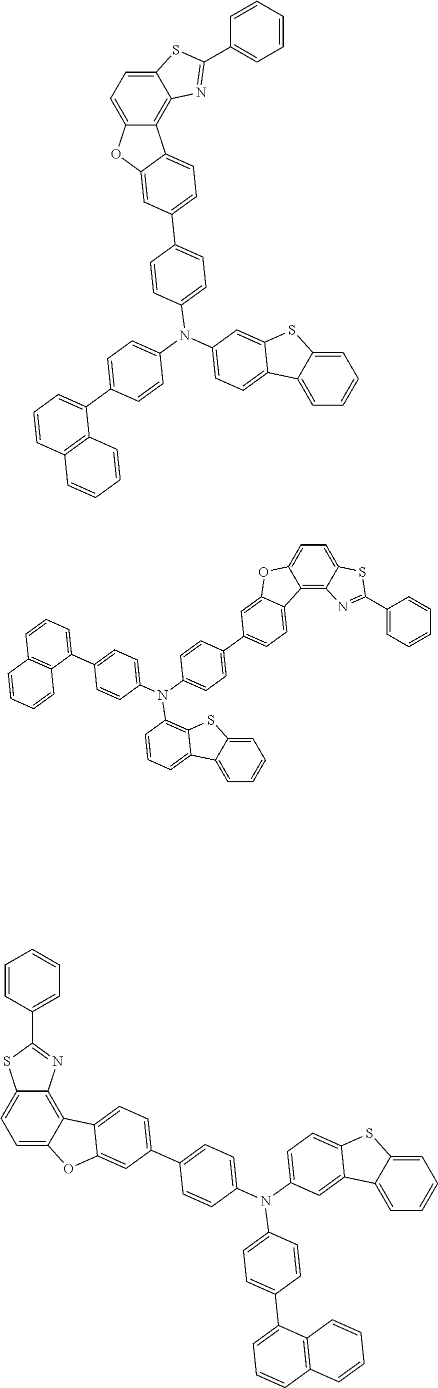 [SMILES: c1ccc(-c2nc3c(ccc4oc5cc(-c6ccc(N(c7ccc(-c8cccc9ccccc89)cc7)c7ccc8c(c7)sc7ccccc78)cc6)ccc5c43)s2)cc1 ; c1ccc(-c2nc3c(ccc4oc5cc(-c6ccc(N(c7ccc(-c8cccc9ccccc89)cc7)c7cccc8c7sc7ccccc78)cc6)ccc5c43)s2)cc1 ; c1ccc(-c2nc3c(ccc4oc5cc(-c6ccc(N(c7ccc(-c8cccc9ccccc89)cc7)c7ccc8sc9ccccc9c8c7)cc6)ccc5c43)s2)cc1]

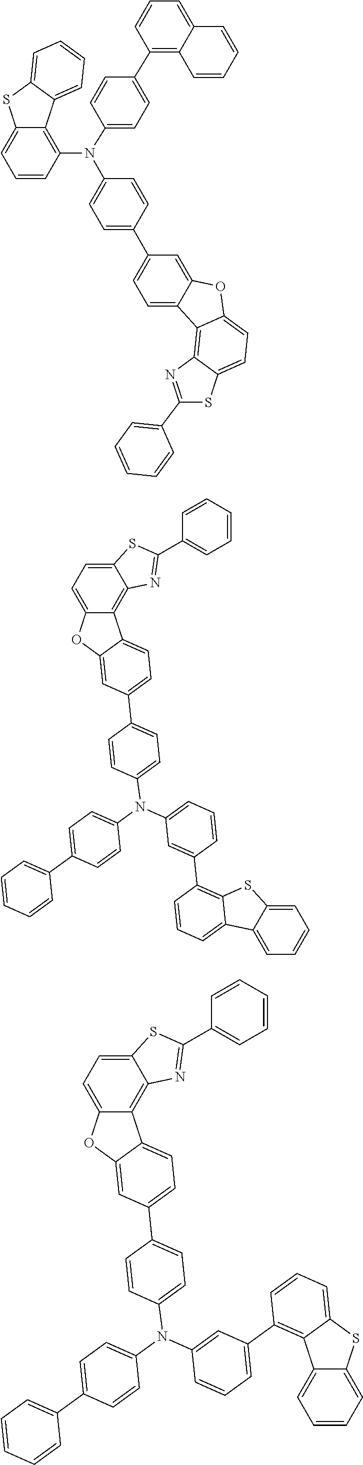

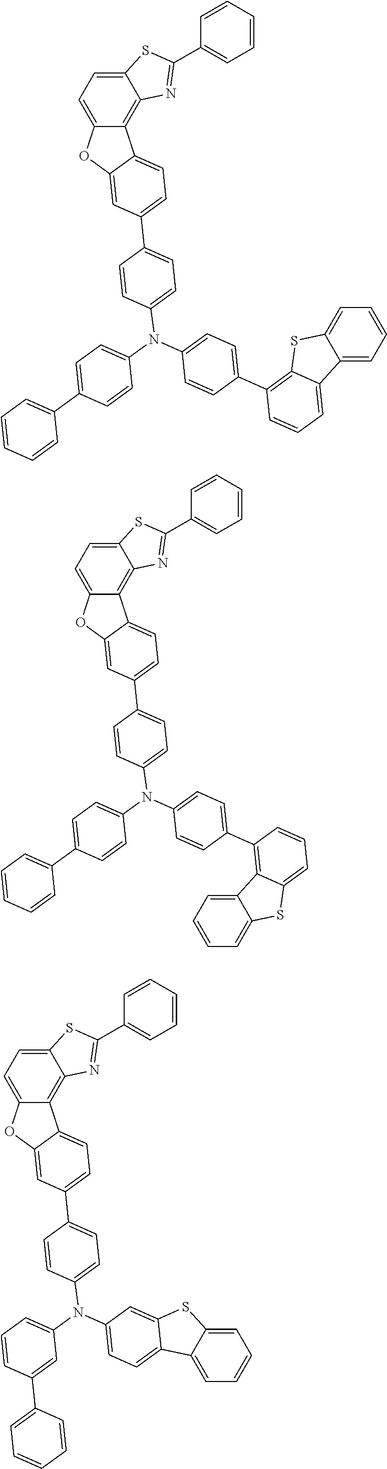 [SMILES: c1ccc(-c2ccc(N(c3ccc(-c4ccc5c(c4)oc4ccc6sc(-c7ccccc7)nc6c45)cc3)c3ccc(-c4cccc5c4sc4ccccc45)cc3)cc2)cc1 ; c1ccc(-c2ccc(N(c3ccc(-c4ccc5c(c4)oc4ccc6sc(-c7ccccc7)nc6c45)cc3)c3ccc(-c4cccc5sc6ccccc6c45)cc3)cc2)cc1 ; c1ccc(-c2cccc(N(c3ccc(-c4ccc5c(c4)oc4ccc6sc(-c7ccccc7)nc6c45)cc3)c3ccc4c(c3)sc3ccccc34)c2)cc1]

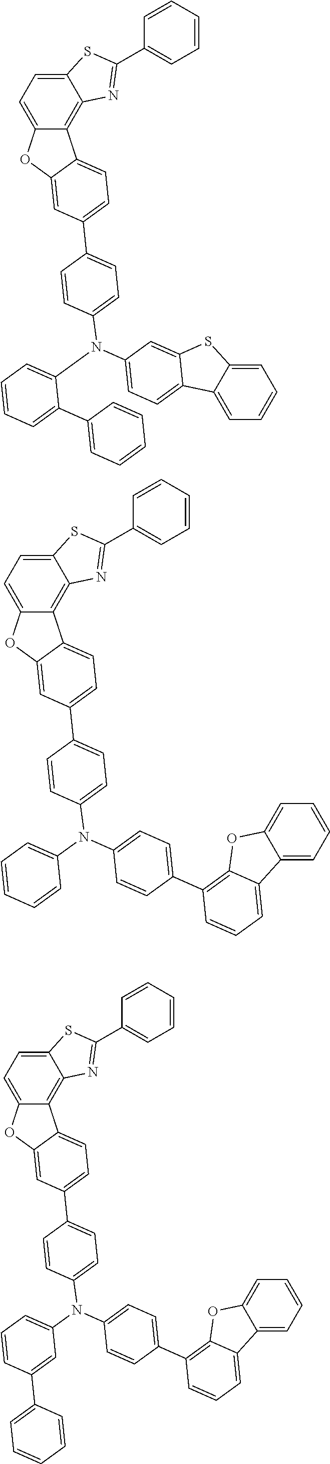

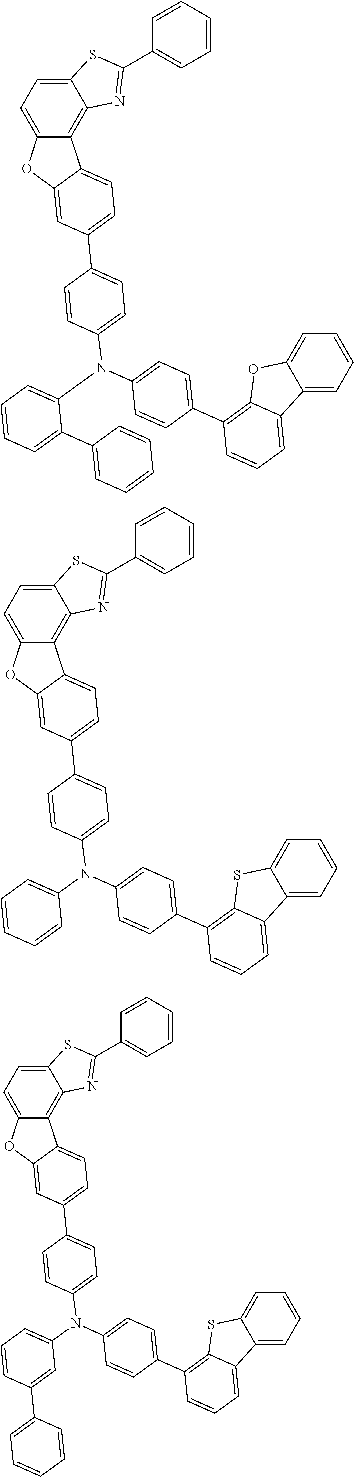

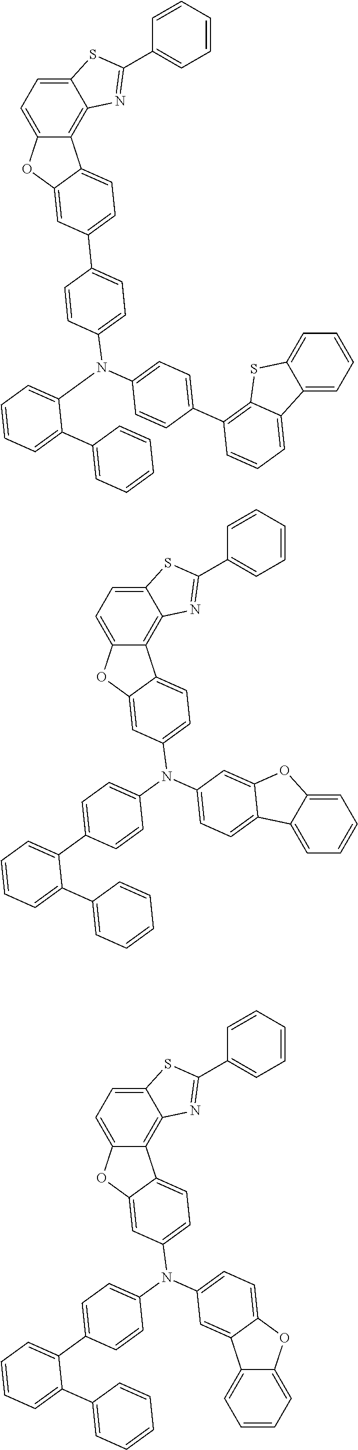

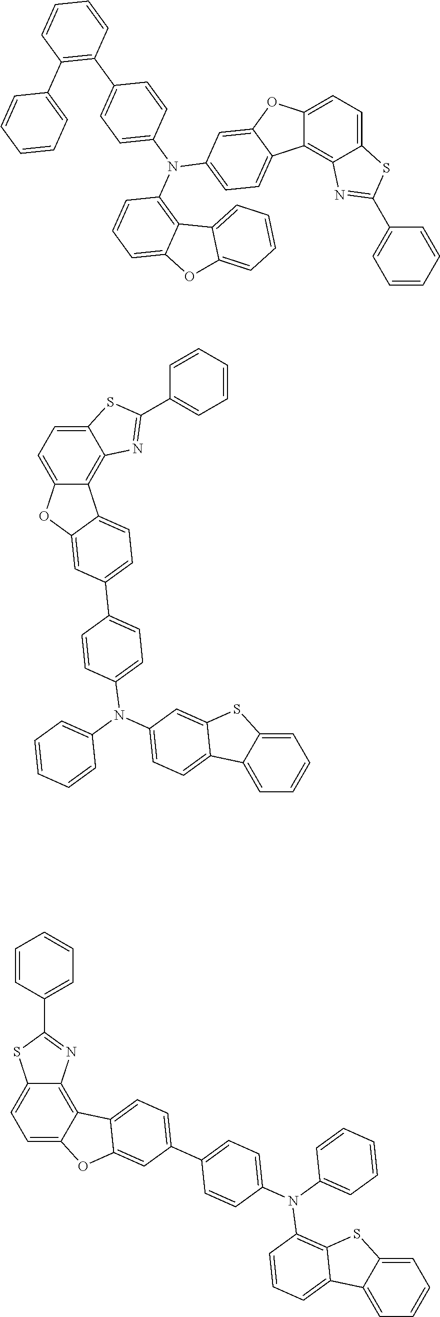 [SMILES: c1ccc(-c2nc3c(ccc4oc5cc(N(c6ccc(-c7ccccc7-c7ccccc7)cc6)c6cccc7oc8ccccc8c67)ccc5c43)s2)cc1 ; c1ccc(-c2nc3c(ccc4oc5cc(-c6ccc(N(c7ccccc7)c7ccc8c(c7)sc7ccccc78)cc6)ccc5c43)s2)cc1 ; c1ccc(-c2nc3c(ccc4oc5cc(-c6ccc(N(c7ccccc7)c7cccc8c7sc7ccccc78)cc6)ccc5c43)s2)cc1]

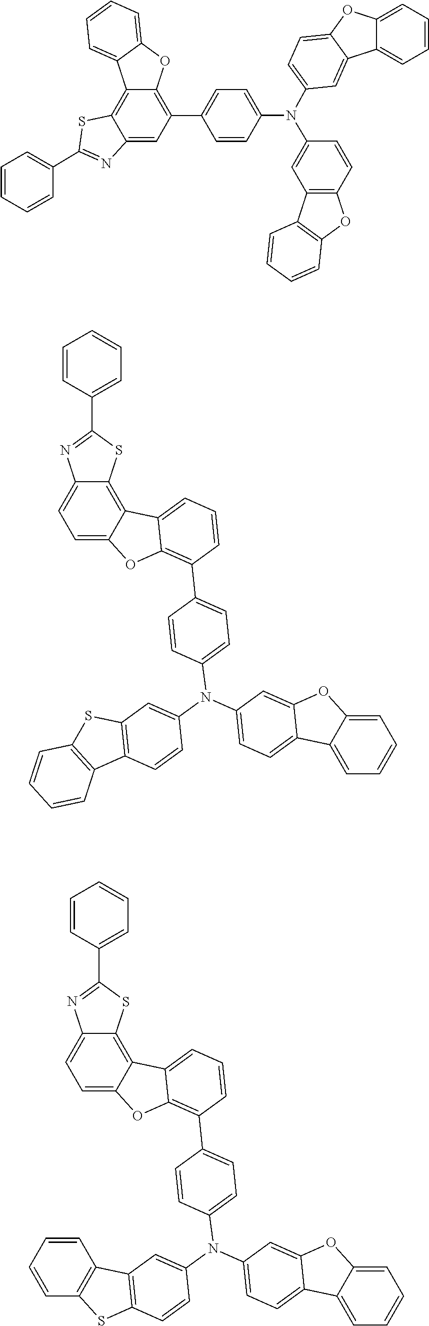 [SMILES: c1ccc(-c2nc3cc(-c4ccc(N(c5ccc6oc7ccccc7c6c5)c5ccc6oc7ccccc7c6c5)cc4)c4oc5ccccc5c4c3s2)cc1 ; c1ccc(-c2nc3ccc4oc5c(-c6ccc(N(c7ccc8c(c7)oc7ccccc78)c7ccc8c(c7)sc7ccccc78)cc6)cccc5c4c3s2)cc1 ; c1ccc(-c2nc3ccc4oc5c(-c6ccc(N(c7ccc8c(c7)oc7ccccc78)c7ccc8sc9ccccc9c8c7)cc6)cccc5c4c3s2)cc1]

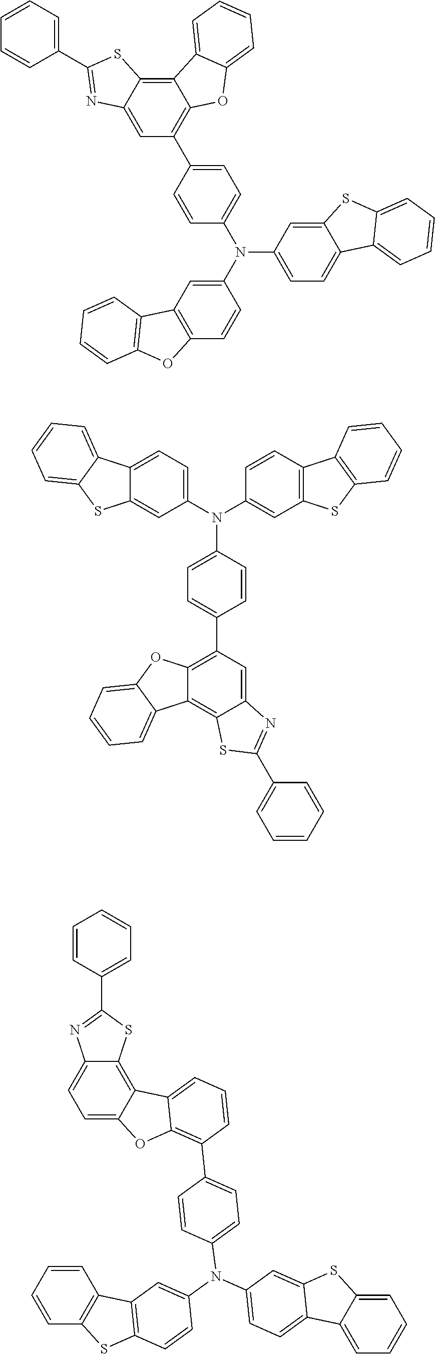 [SMILES: c1ccc(-c2nc3cc(-c4ccc(N(c5ccc6c(c5)sc5ccccc56)c5ccc6oc7ccccc7c6c5)cc4)c4oc5ccccc5c4c3s2)cc1 ; c1ccc(-c2nc3cc(-c4ccc(N(c5ccc6c(c5)sc5ccccc56)c5ccc6c(c5)sc5ccccc56)cc4)c4oc5ccccc5c4c3s2)cc1 ; c1ccc(-c2nc3ccc4oc5c(-c6ccc(N(c7ccc8c(c7)sc7ccccc78)c7ccc8sc9ccccc9c8c7)cc6)cccc5c4c3s2)cc1]

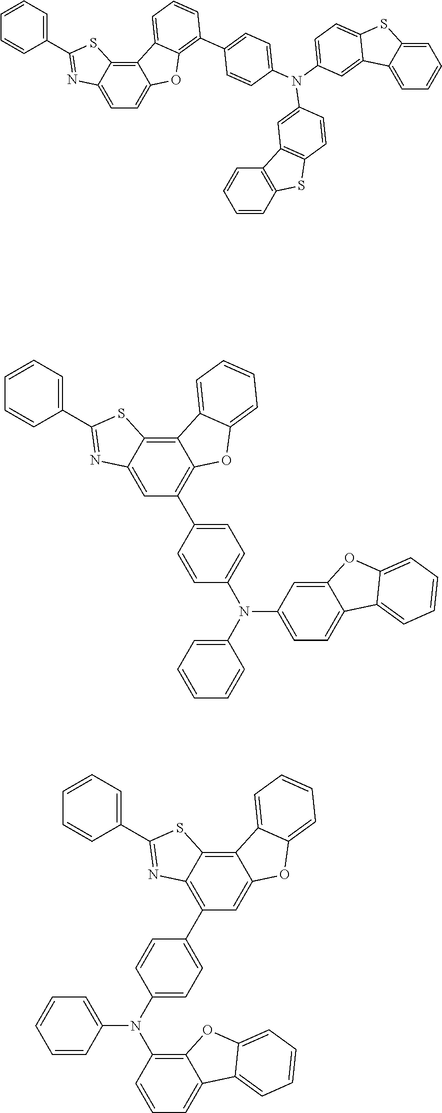 [SMILES: c1ccc(-c2nc3ccc4oc5c(-c6ccc(N(c7ccc8sc9ccccc9c8c7)c7ccc8sc9ccccc9c8c7)cc6)cccc5c4c3s2)cc1 ; c1ccc(-c2nc3cc(-c4ccc(N(c5ccccc5)c5ccc6c(c5)oc5ccccc56)cc4)c4oc5ccccc5c4c3s2)cc1 ; c1ccc(-c2nc3c(-c4ccc(N(c5ccccc5)c5cccc6c5oc5ccccc56)cc4)cc4oc5ccccc5c4c3s2)cc1]

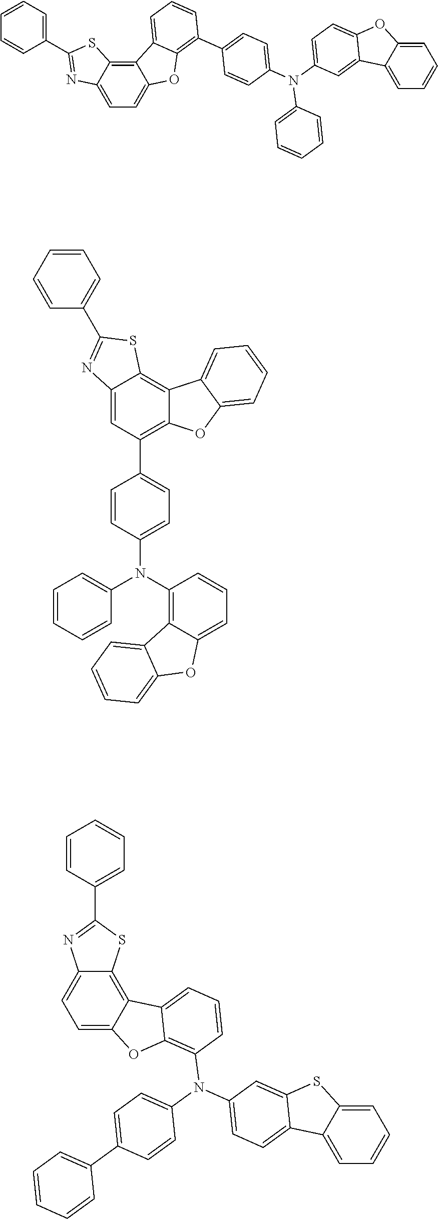 [SMILES: c1ccc(-c2nc3ccc4oc5c(-c6ccc(N(c7ccccc7)c7ccc8oc9ccccc9c8c7)cc6)cccc5c4c3s2)cc1 ; c1ccc(-c2nc3cc(-c4ccc(N(c5ccccc5)c5cccc6oc7ccccc7c56)cc4)c4oc5ccccc5c4c3s2)cc1 ; c1ccc(-c2ccc(N(c3ccc4c(c3)sc3ccccc34)c3cccc4c3oc3ccc5nc(-c6ccccc6)sc5c34)cc2)cc1]

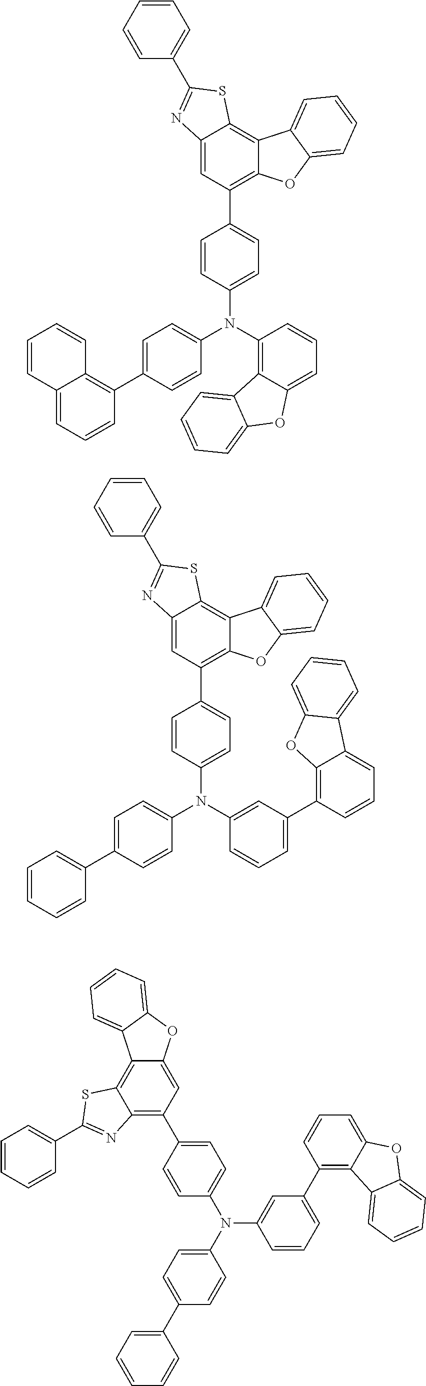

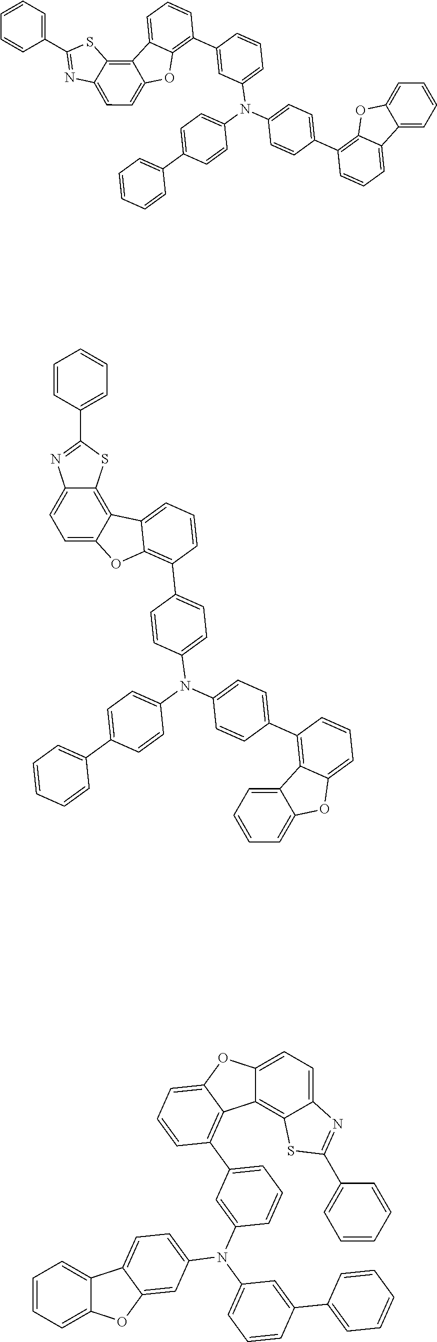 [SMILES: c1ccc(-c2ccc(N(c3ccc(-c4cccc5c4oc4ccccc45)cc3)c3cccc(-c4cccc5c4oc4ccc6nc(-c7ccccc7)sc6c45)c3)cc2)cc1 ; c1ccc(-c2ccc(N(c3ccc(-c4cccc5c4oc4ccc6nc(-c7ccccc7)sc6c45)cc3)c3ccc(-c4cccc5oc6ccccc6c45)cc3)cc2)cc1 ; c1ccc(-c2cccc(N(c3cccc(-c4cccc5oc6ccc7nc(-c8ccccc8)sc7c6c45)c3)c3ccc4c(c3)oc3ccccc34)c2)cc1]

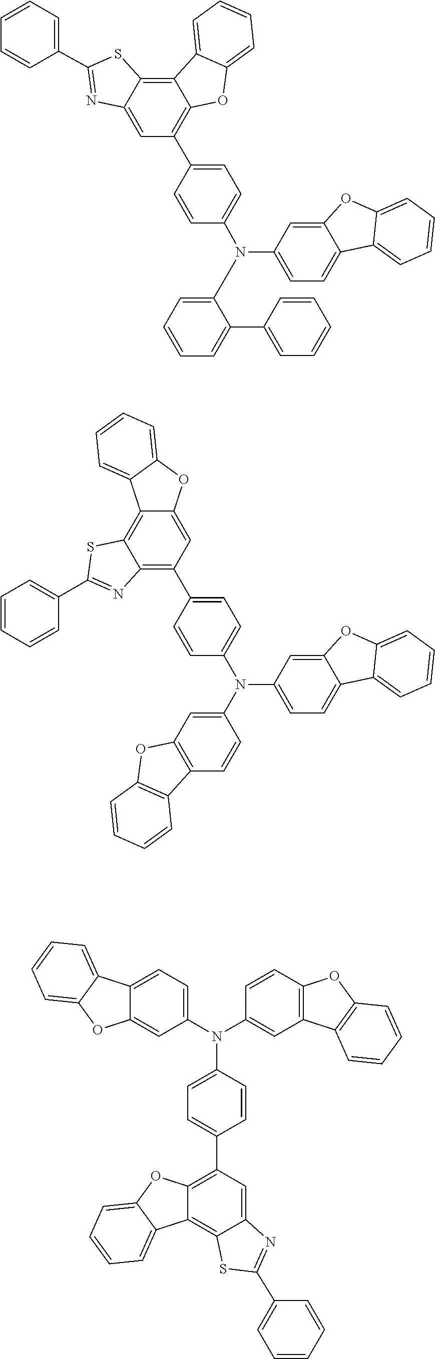 [SMILES: c1ccc(-c2nc3cc(-c4ccc(N(c5ccc6c(c5)oc5ccccc56)c5ccccc5-c5ccccc5)cc4)c4oc5ccccc5c4c3s2)cc1 ; c1ccc(-c2nc3c(-c4ccc(N(c5ccc6c(c5)oc5ccccc56)c5ccc6c(c5)oc5ccccc56)cc4)cc4oc5ccccc5c4c3s2)cc1 ; c1ccc(-c2nc3cc(-c4ccc(N(c5ccc6c(c5)oc5ccccc56)c5ccc6oc7ccccc7c6c5)cc4)c4oc5ccccc5c4c3s2)cc1]

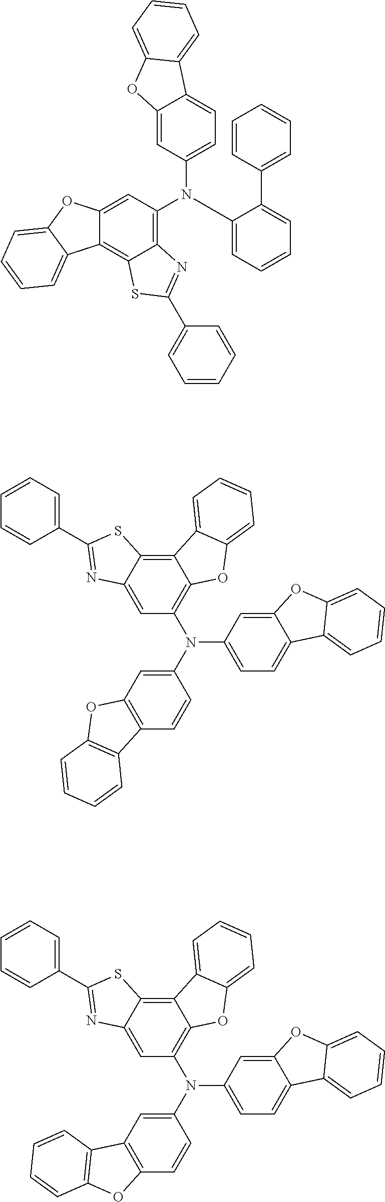 [SMILES: c1ccc(-c2nc3c(N(c4ccc5c(c4)oc4ccccc45)c4ccccc4-c4ccccc4)cc4oc5ccccc5c4c3s2)cc1 ; c1ccc(-c2nc3cc(N(c4ccc5c(c4)oc4ccccc45)c4ccc5c(c4)oc4ccccc45)c4oc5ccccc5c4c3s2)cc1 ; c1ccc(-c2nc3cc(N(c4ccc5c(c4)oc4ccccc45)c4ccc5oc6ccccc6c5c4)c4oc5ccccc5c4c3s2)cc1]

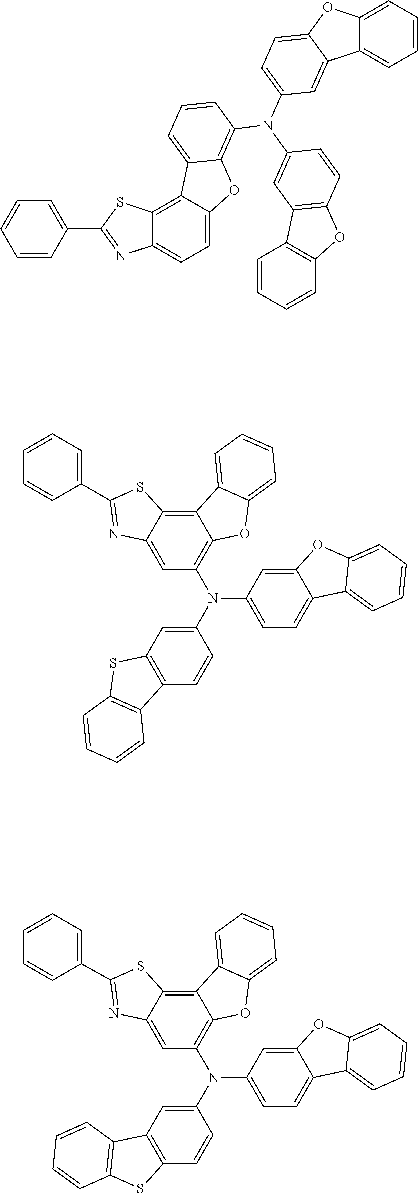 [SMILES: c1ccc(-c2nc3ccc4oc5c(N(c6ccc7oc8ccccc8c7c6)c6ccc7oc8ccccc8c7c6)cccc5c4c3s2)cc1 ; c1ccc(-c2nc3cc(N(c4ccc5c(c4)oc4ccccc45)c4ccc5c(c4)sc4ccccc45)c4oc5ccccc5c4c3s2)cc1 ; c1ccc(-c2nc3cc(N(c4ccc5c(c4)oc4ccccc45)c4ccc5sc6ccccc6c5c4)c4oc5ccccc5c4c3s2)cc1]

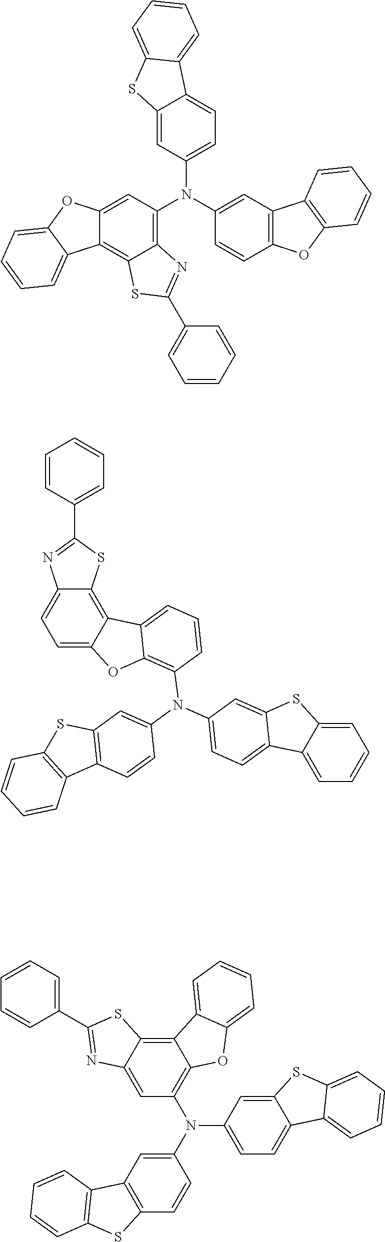 [SMILES: c1ccc(-c2nc3c(N(c4ccc5c(c4)sc4ccccc45)c4ccc5oc6ccccc6c5c4)cc4oc5ccccc5c4c3s2)cc1 ; c1ccc(-c2nc3ccc4oc5c(N(c6ccc7c(c6)sc6ccccc67)c6ccc7c(c6)sc6ccccc67)cccc5c4c3s2)cc1 ; c1ccc(-c2nc3cc(N(c4ccc5c(c4)sc4ccccc45)c4ccc5sc6ccccc6c5c4)c4oc5ccccc5c4c3s2)cc1]

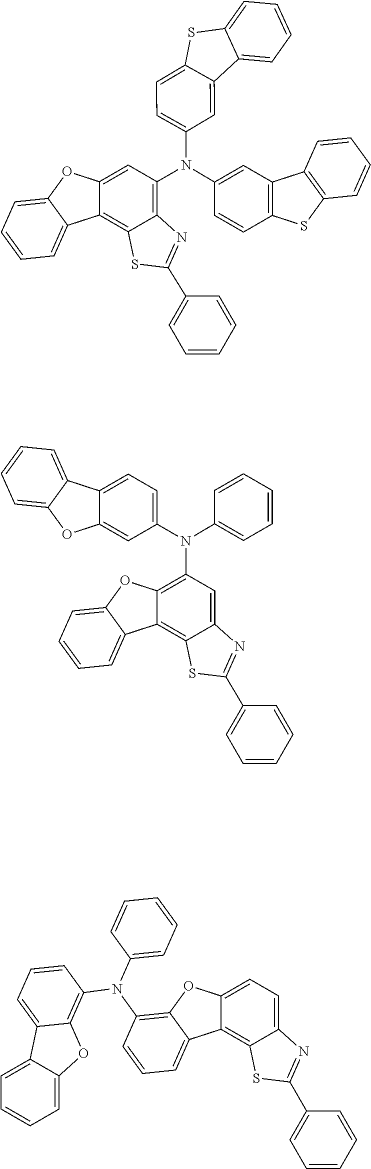 [SMILES: c1ccc(-c2nc3c(N(c4ccc5sc6ccccc6c5c4)c4ccc5sc6ccccc6c5c4)cc4oc5ccccc5c4c3s2)cc1 ; c1ccc(-c2nc3cc(N(c4ccccc4)c4ccc5c(c4)oc4ccccc45)c4oc5ccccc5c4c3s2)cc1 ; c1ccc(-c2nc3ccc4oc5c(N(c6ccccc6)c6cccc7c6oc6ccccc67)cccc5c4c3s2)cc1]

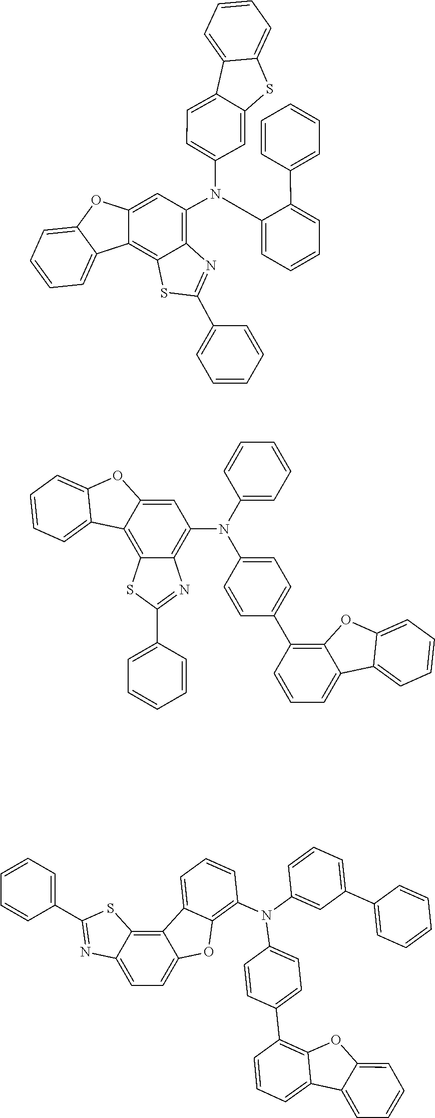 [SMILES: c1ccc(-c2nc3c(N(c4ccc5c(c4)sc4ccccc45)c4ccccc4-c4ccccc4)cc4oc5ccccc5c4c3s2)cc1 ; c1ccc(-c2nc3c(N(c4ccccc4)c4ccc(-c5cccc6c5oc5ccccc56)cc4)cc4oc5ccccc5c4c3s2)cc1 ; c1ccc(-c2cccc(N(c3ccc(-c4cccc5c4oc4ccccc45)cc3)c3cccc4c3oc3ccc5nc(-c6ccccc6)sc5c34)c2)cc1]

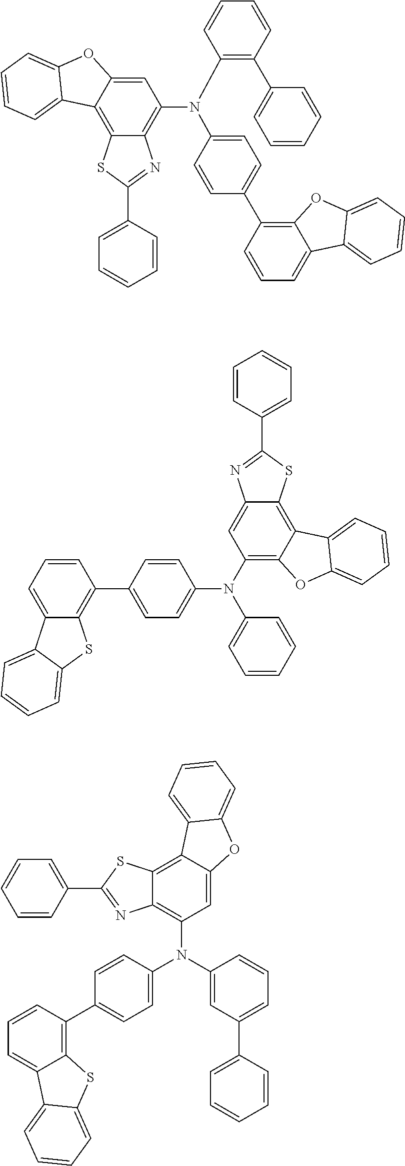 [SMILES: c1ccc(-c2nc3c(N(c4ccc(-c5cccc6c5oc5ccccc56)cc4)c4ccccc4-c4ccccc4)cc4oc5ccccc5c4c3s2)cc1 ; c1ccc(-c2nc3cc(N(c4ccccc4)c4ccc(-c5cccc6c5sc5ccccc56)cc4)c4oc5ccccc5c4c3s2)cc1 ; c1ccc(-c2cccc(N(c3ccc(-c4cccc5c4sc4ccccc45)cc3)c3cc4oc5ccccc5c4c4sc(-c5ccccc5)nc34)c2)cc1]

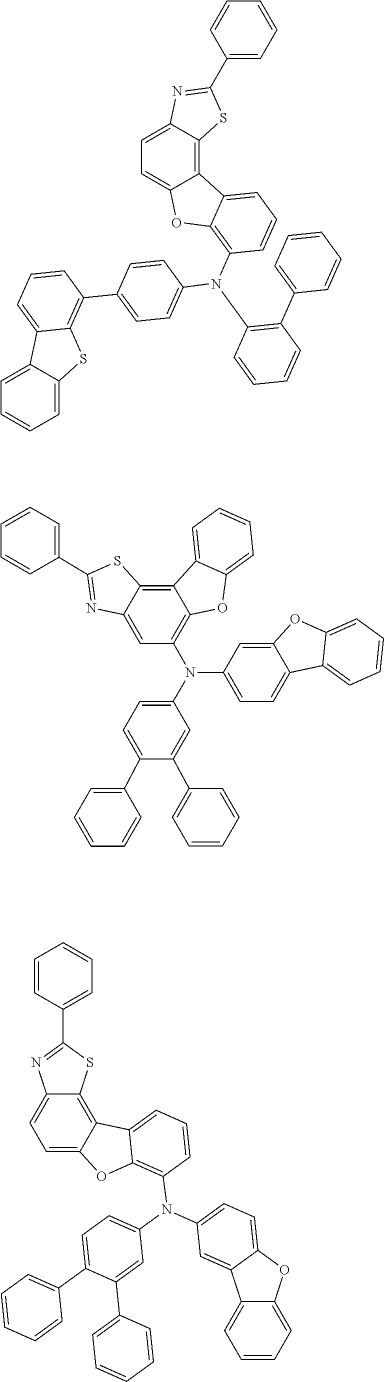 [SMILES: c1ccc(-c2nc3ccc4oc5c(N(c6ccc(-c7cccc8c7sc7ccccc78)cc6)c6ccccc6-c6ccccc6)cccc5c4c3s2)cc1 ; c1ccc(-c2nc3cc(N(c4ccc(-c5ccccc5)c(-c5ccccc5)c4)c4ccc5c(c4)oc4ccccc45)c4oc5ccccc5c4c3s2)cc1 ; c1ccc(-c2nc3ccc4oc5c(N(c6ccc(-c7ccccc7)c(-c7ccccc7)c6)c6ccc7oc8ccccc8c7c6)cccc5c4c3s2)cc1]

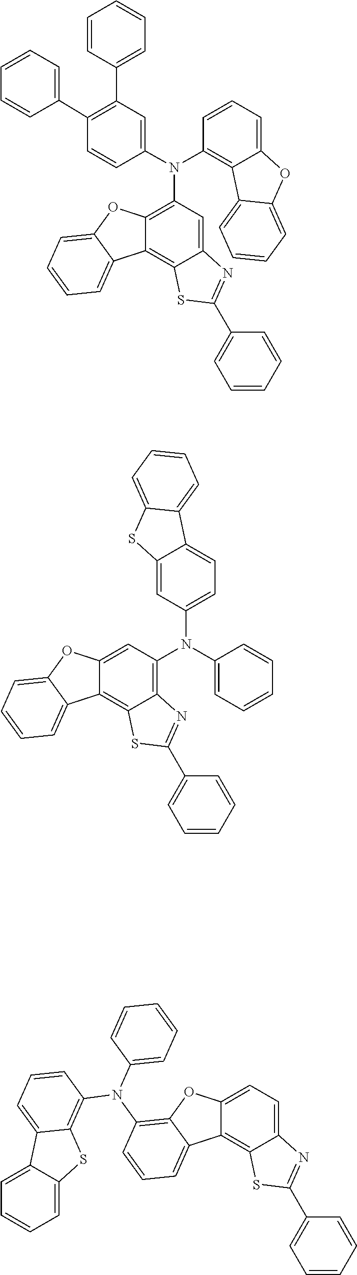 [SMILES: c1ccc(-c2nc3cc(N(c4ccc(-c5ccccc5)c(-c5ccccc5)c4)c4cccc5oc6ccccc6c45)c4oc5ccccc5c4c3s2)cc1 ; c1ccc(-c2nc3c(N(c4ccccc4)c4ccc5c(c4)sc4ccccc45)cc4oc5ccccc5c4c3s2)cc1 ; c1ccc(-c2nc3ccc4oc5c(N(c6ccccc6)c6cccc7c6sc6ccccc67)cccc5c4c3s2)cc1]

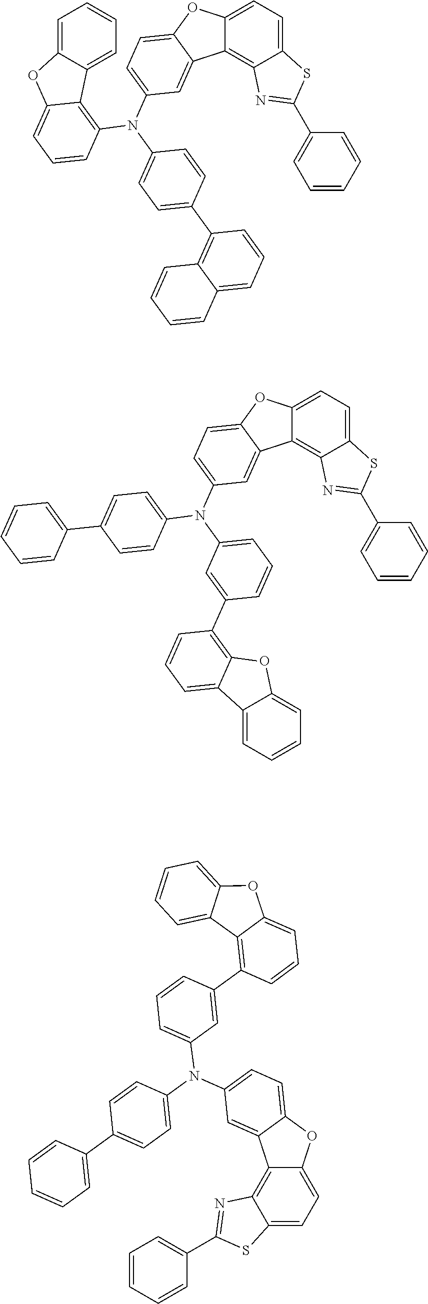 [SMILES: c1ccc(-c2nc3c(ccc4oc5ccc(N(c6ccc(-c7cccc8ccccc78)cc6)c6cccc7oc8ccccc8c67)cc5c43)s2)cc1 ; c1ccc(-c2ccc(N(c3cccc(-c4cccc5c4oc4ccccc45)c3)c3ccc4oc5ccc6sc(-c7ccccc7)nc6c5c4c3)cc2)cc1 ; c1ccc(-c2ccc(N(c3cccc(-c4cccc5oc6ccccc6c45)c3)c3ccc4oc5ccc6sc(-c7ccccc7)nc6c5c4c3)cc2)cc1]

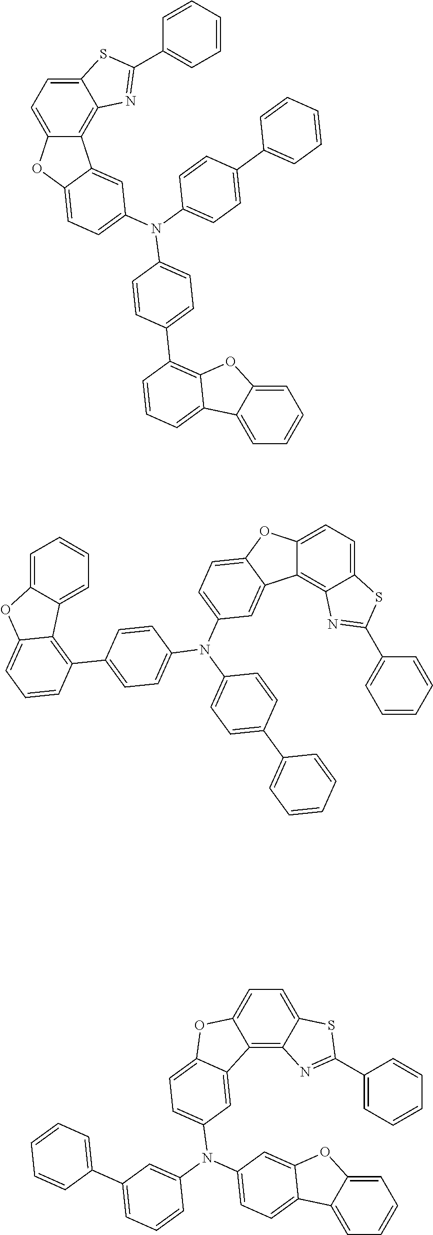 [SMILES: c1ccc(-c2ccc(N(c3ccc(-c4cccc5c4oc4ccccc45)cc3)c3ccc4oc5ccc6sc(-c7ccccc7)nc6c5c4c3)cc2)cc1 ; c1ccc(-c2ccc(N(c3ccc(-c4cccc5oc6ccccc6c45)cc3)c3ccc4oc5ccc6sc(-c7ccccc7)nc6c5c4c3)cc2)cc1 ; c1ccc(-c2cccc(N(c3ccc4c(c3)oc3ccccc34)c3ccc4oc5ccc6sc(-c7ccccc7)nc6c5c4c3)c2)cc1]

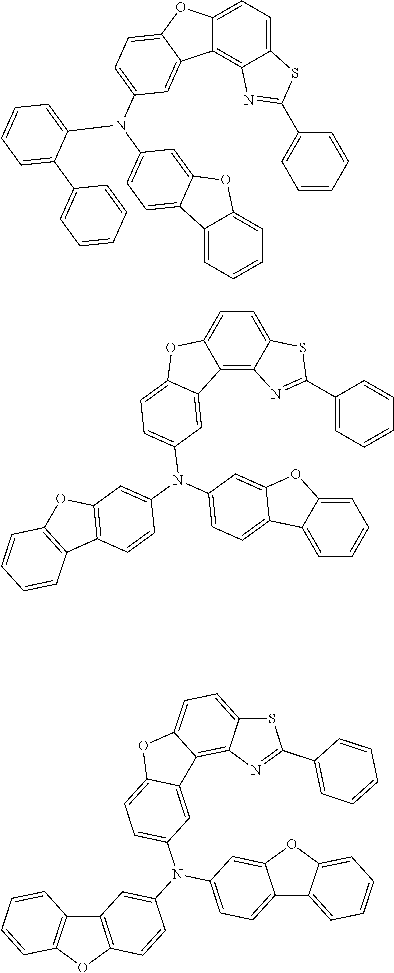

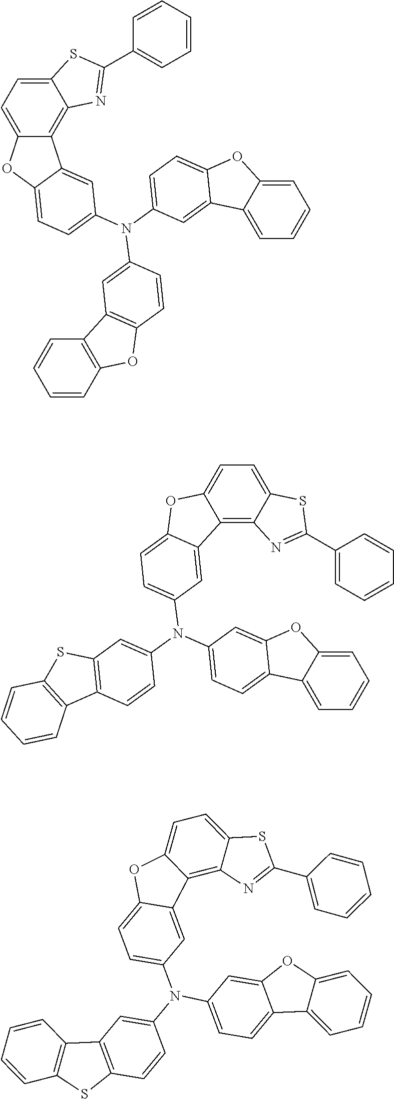 [SMILES: c1ccc(-c2nc3c(ccc4oc5ccc(N(c6ccc7oc8ccccc8c7c6)c6ccc7oc8ccccc8c7c6)cc5c43)s2)cc1 ; c1ccc(-c2nc3c(ccc4oc5ccc(N(c6ccc7c(c6)oc6ccccc67)c6ccc7c(c6)sc6ccccc67)cc5c43)s2)cc1 ; c1ccc(-c2nc3c(ccc4oc5ccc(N(c6ccc7c(c6)oc6ccccc67)c6ccc7sc8ccccc8c7c6)cc5c43)s2)cc1]

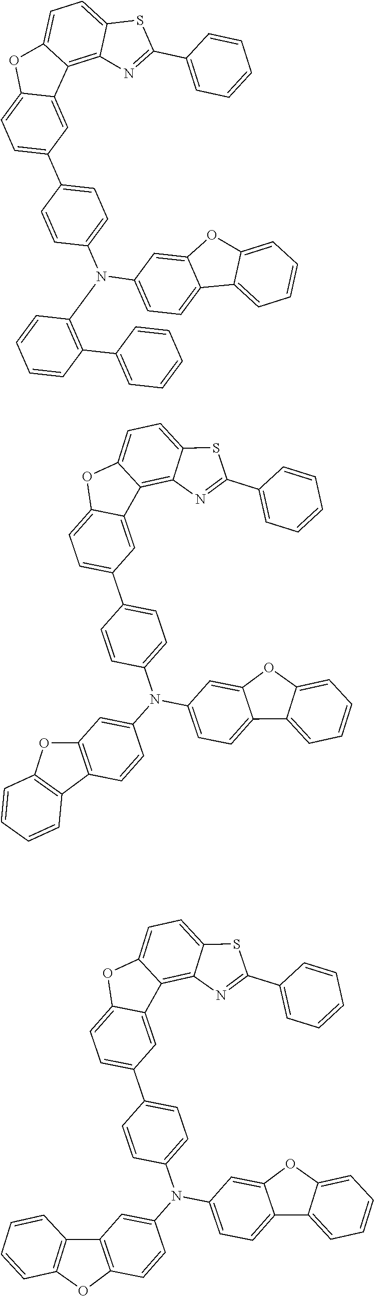

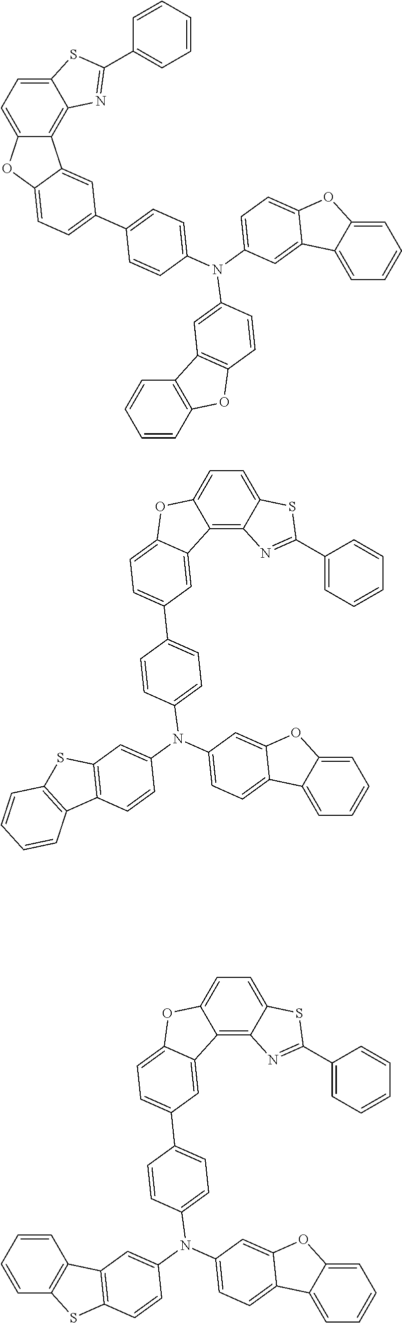 [SMILES: c1ccc(-c2nc3c(ccc4oc5ccc(-c6ccc(N(c7ccc8oc9ccccc9c8c7)c7ccc8oc9ccccc9c8c7)cc6)cc5c43)s2)cc1 ; c1ccc(-c2nc3c(ccc4oc5ccc(-c6ccc(N(c7ccc8c(c7)oc7ccccc78)c7ccc8c(c7)sc7ccccc78)cc6)cc5c43)s2)cc1 ; c1ccc(-c2nc3c(ccc4oc5ccc(-c6ccc(N(c7ccc8c(c7)oc7ccccc78)c7ccc8sc9ccccc9c8c7)cc6)cc5c43)s2)cc1]

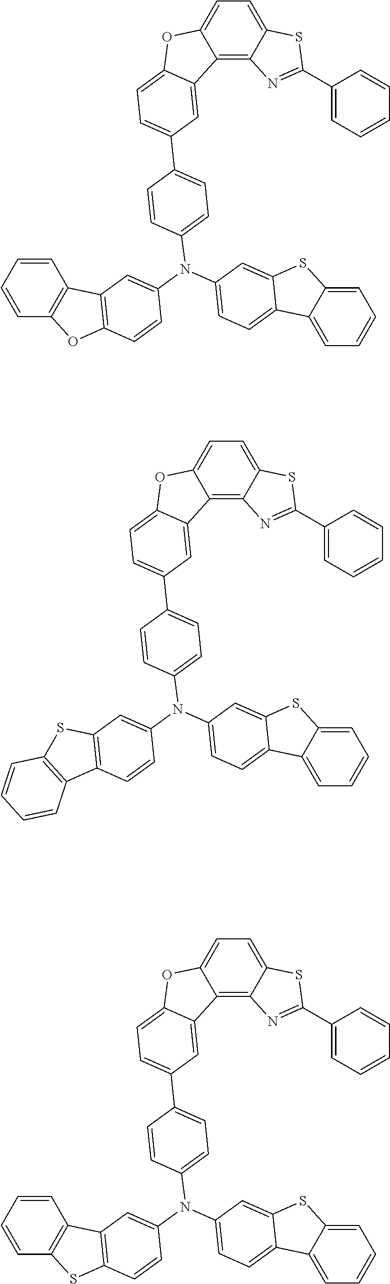 [SMILES: c1ccc(-c2nc3c(ccc4oc5ccc(-c6ccc(N(c7ccc8c(c7)sc7ccccc78)c7ccc8oc9ccccc9c8c7)cc6)cc5c43)s2)cc1 ; c1ccc(-c2nc3c(ccc4oc5ccc(-c6ccc(N(c7ccc8c(c7)sc7ccccc78)c7ccc8c(c7)sc7ccccc78)cc6)cc5c43)s2)cc1 ; c1ccc(-c2nc3c(ccc4oc5ccc(-c6ccc(N(c7ccc8c(c7)sc7ccccc78)c7ccc8sc9ccccc9c8c7)cc6)cc5c43)s2)cc1]

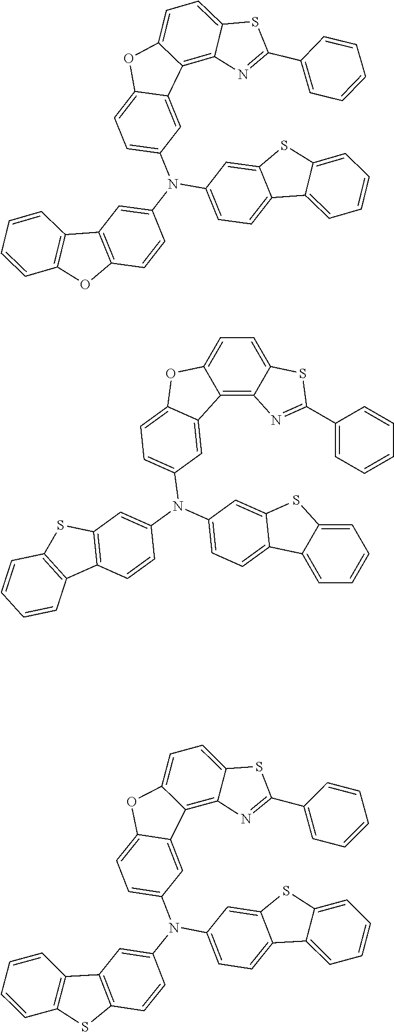 [SMILES: c1ccc(-c2nc3c(ccc4oc5ccc(N(c6ccc7c(c6)sc6ccccc67)c6ccc7oc8ccccc8c7c6)cc5c43)s2)cc1 ; c1ccc(-c2nc3c(ccc4oc5ccc(N(c6ccc7c(c6)sc6ccccc67)c6ccc7c(c6)sc6ccccc67)cc5c43)s2)cc1 ; c1ccc(-c2nc3c(ccc4oc5ccc(N(c6ccc7c(c6)sc6ccccc67)c6ccc7sc8ccccc8c7c6)cc5c43)s2)cc1]

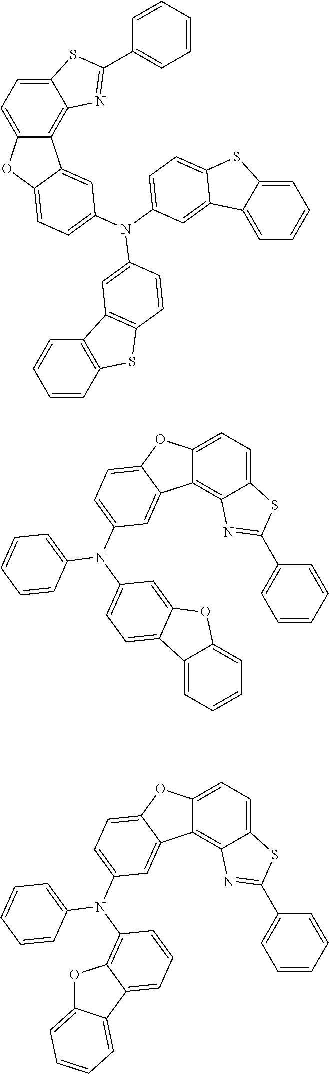 [SMILES: c1ccc(-c2nc3c(ccc4oc5ccc(N(c6ccc7sc8ccccc8c7c6)c6ccc7sc8ccccc8c7c6)cc5c43)s2)cc1 ; c1ccc(-c2nc3c(ccc4oc5ccc(N(c6ccccc6)c6ccc7c(c6)oc6ccccc67)cc5c43)s2)cc1 ; c1ccc(-c2nc3c(ccc4oc5ccc(N(c6ccccc6)c6cccc7c6oc6ccccc67)cc5c43)s2)cc1]

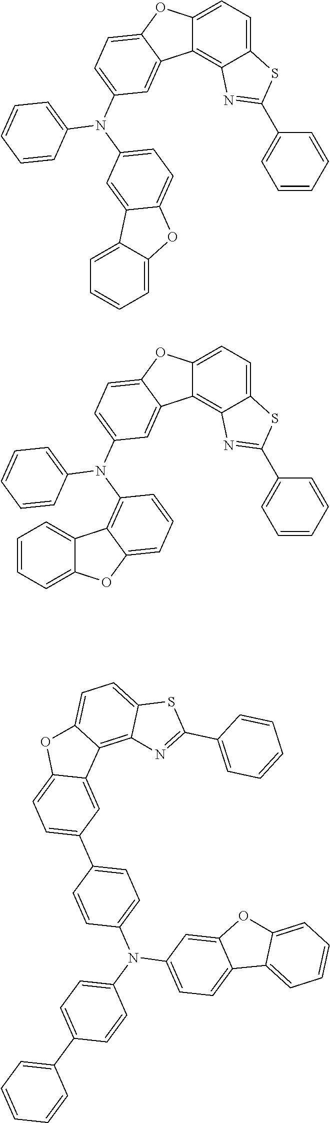 [SMILES: c1ccc(-c2nc3c(ccc4oc5ccc(N(c6ccccc6)c6ccc7oc8ccccc8c7c6)cc5c43)s2)cc1 ; c1ccc(-c2nc3c(ccc4oc5ccc(N(c6ccccc6)c6cccc7oc8ccccc8c67)cc5c43)s2)cc1 ; c1ccc(-c2ccc(N(c3ccc(-c4ccc5oc6ccc7sc(-c8ccccc8)nc7c6c5c4)cc3)c3ccc4c(c3)oc3ccccc34)cc2)cc1]

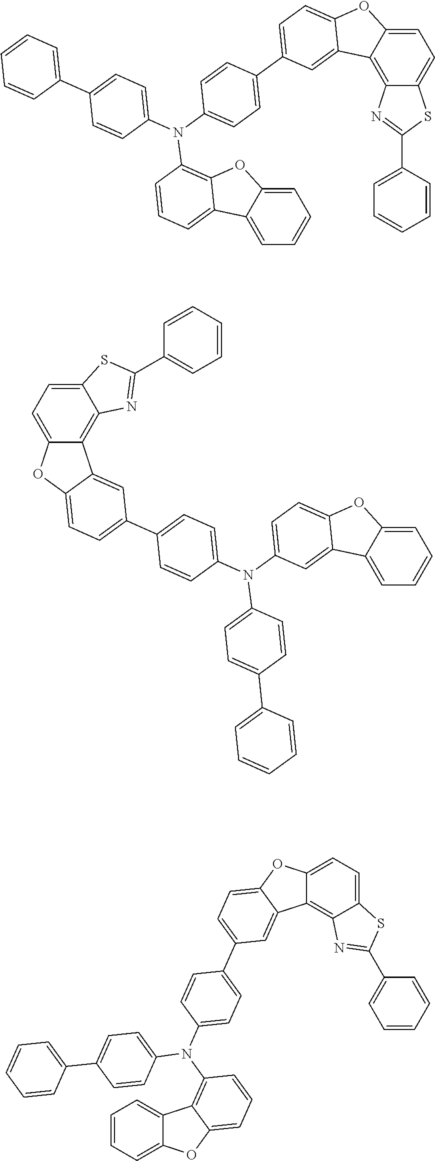 [SMILES: c1ccc(-c2ccc(N(c3ccc(-c4ccc5oc6ccc7sc(-c8ccccc8)nc7c6c5c4)cc3)c3cccc4c3oc3ccccc34)cc2)cc1 ; c1ccc(-c2ccc(N(c3ccc(-c4ccc5oc6ccc7sc(-c8ccccc8)nc7c6c5c4)cc3)c3ccc4oc5ccccc5c4c3)cc2)cc1 ; c1ccc(-c2ccc(N(c3ccc(-c4ccc5oc6ccc7sc(-c8ccccc8)nc7c6c5c4)cc3)c3cccc4oc5ccccc5c34)cc2)cc1]

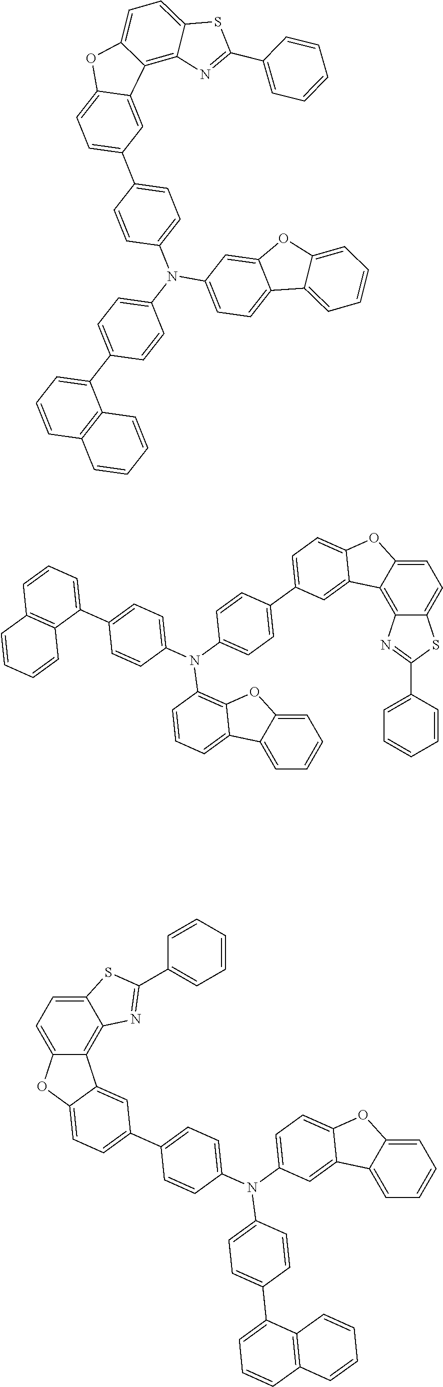 [SMILES: c1ccc(-c2nc3c(ccc4oc5ccc(-c6ccc(N(c7ccc(-c8cccc9ccccc89)cc7)c7ccc8c(c7)oc7ccccc78)cc6)cc5c43)s2)cc1 ; c1ccc(-c2nc3c(ccc4oc5ccc(-c6ccc(N(c7ccc(-c8cccc9ccccc89)cc7)c7cccc8c7oc7ccccc78)cc6)cc5c43)s2)cc1 ; c1ccc(-c2nc3c(ccc4oc5ccc(-c6ccc(N(c7ccc(-c8cccc9ccccc89)cc7)c7ccc8oc9ccccc9c8c7)cc6)cc5c43)s2)cc1]

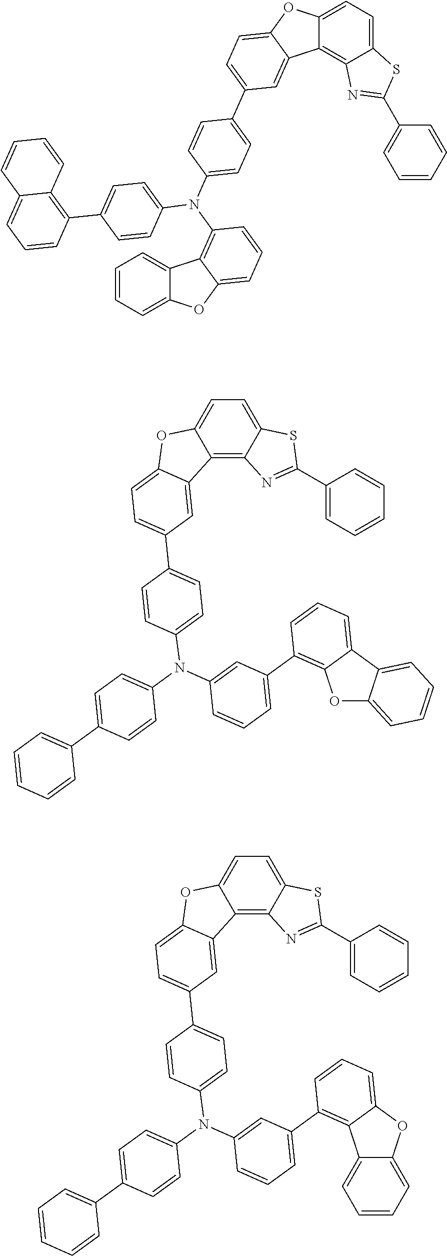 [SMILES: c1ccc(-c2nc3c(ccc4oc5ccc(-c6ccc(N(c7ccc(-c8cccc9ccccc89)cc7)c7cccc8oc9ccccc9c78)cc6)cc5c43)s2)cc1 ; c1ccc(-c2ccc(N(c3ccc(-c4ccc5oc6ccc7sc(-c8ccccc8)nc7c6c5c4)cc3)c3cccc(-c4cccc5c4oc4ccccc45)c3)cc2)cc1 ; c1ccc(-c2ccc(N(c3ccc(-c4ccc5oc6ccc7sc(-c8ccccc8)nc7c6c5c4)cc3)c3cccc(-c4cccc5oc6ccccc6c45)c3)cc2)cc1]

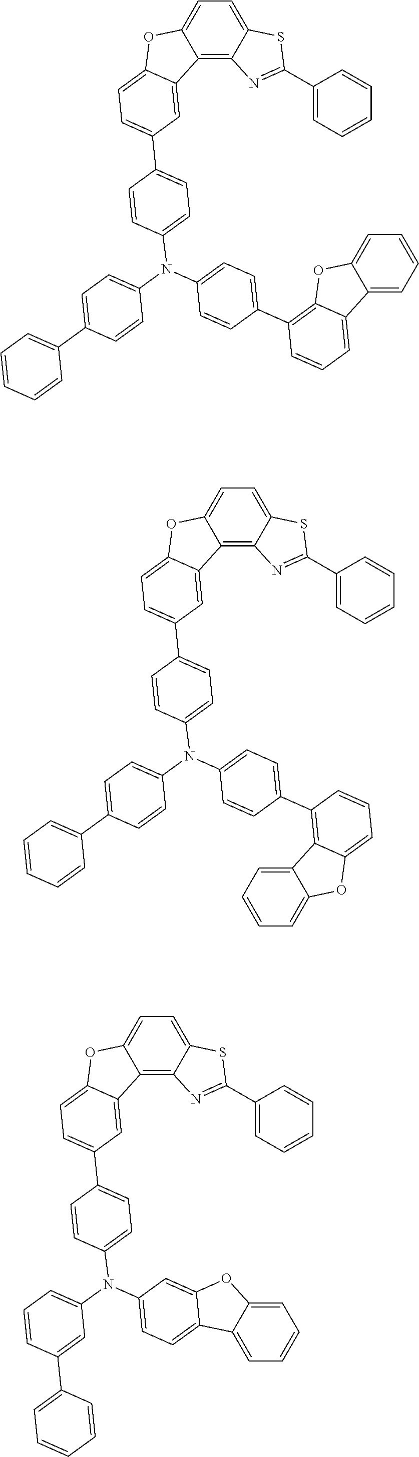 [SMILES: c1ccc(-c2ccc(N(c3ccc(-c4ccc5oc6ccc7sc(-c8ccccc8)nc7c6c5c4)cc3)c3ccc(-c4cccc5c4oc4ccccc45)cc3)cc2)cc1 ; c1ccc(-c2ccc(N(c3ccc(-c4ccc5oc6ccc7sc(-c8ccccc8)nc7c6c5c4)cc3)c3ccc(-c4cccc5oc6ccccc6c45)cc3)cc2)cc1 ; c1ccc(-c2cccc(N(c3ccc(-c4ccc5oc6ccc7sc(-c8ccccc8)nc7c6c5c4)cc3)c3ccc4c(c3)oc3ccccc34)c2)cc1]

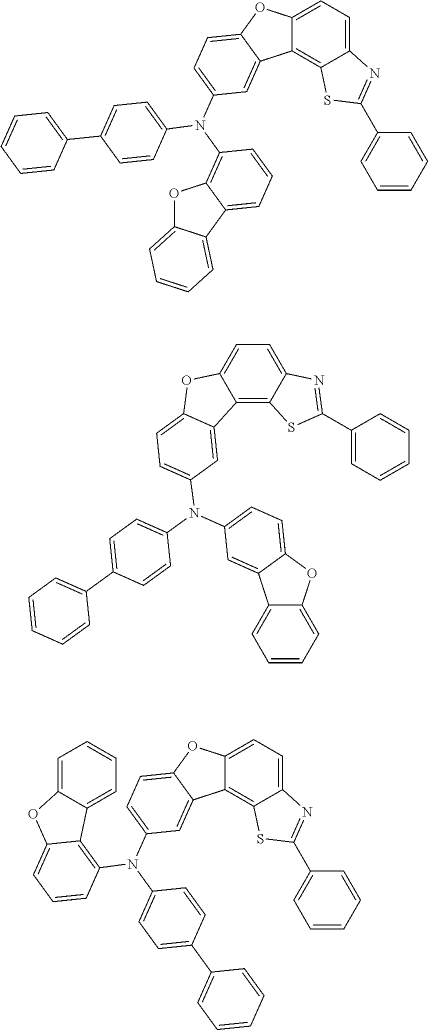 [SMILES: c1ccc(-c2ccc(N(c3ccc4oc5ccc6nc(-c7ccccc7)sc6c5c4c3)c3cccc4c3oc3ccccc34)cc2)cc1 ; c1ccc(-c2ccc(N(c3ccc4oc5ccccc5c4c3)c3ccc4oc5ccc6nc(-c7ccccc7)sc6c5c4c3)cc2)cc1 ; c1ccc(-c2ccc(N(c3ccc4oc5ccc6nc(-c7ccccc7)sc6c5c4c3)c3cccc4oc5ccccc5c34)cc2)cc1]

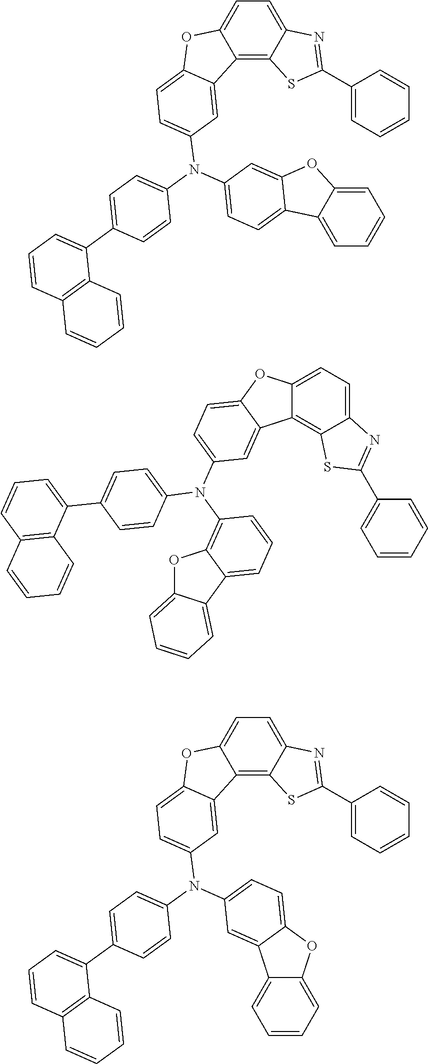 [SMILES: c1ccc(-c2nc3ccc4oc5ccc(N(c6ccc(-c7cccc8ccccc78)cc6)c6ccc7c(c6)oc6ccccc67)cc5c4c3s2)cc1 ; c1ccc(-c2nc3ccc4oc5ccc(N(c6ccc(-c7cccc8ccccc78)cc6)c6cccc7c6oc6ccccc67)cc5c4c3s2)cc1 ; c1ccc(-c2nc3ccc4oc5ccc(N(c6ccc(-c7cccc8ccccc78)cc6)c6ccc7oc8ccccc8c7c6)cc5c4c3s2)cc1]

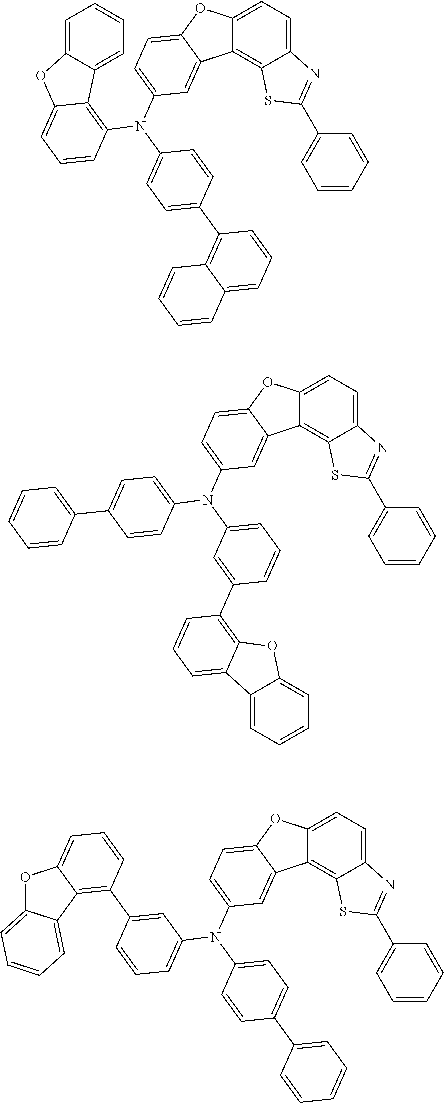 [SMILES: c1ccc(-c2nc3ccc4oc5ccc(N(c6ccc(-c7cccc8ccccc78)cc6)c6cccc7oc8ccccc8c67)cc5c4c3s2)cc1 ; c1ccc(-c2ccc(N(c3cccc(-c4cccc5c4oc4ccccc45)c3)c3ccc4oc5ccc6nc(-c7ccccc7)sc6c5c4c3)cc2)cc1 ; c1ccc(-c2ccc(N(c3cccc(-c4cccc5oc6ccccc6c45)c3)c3ccc4oc5ccc6nc(-c7ccccc7)sc6c5c4c3)cc2)cc1]

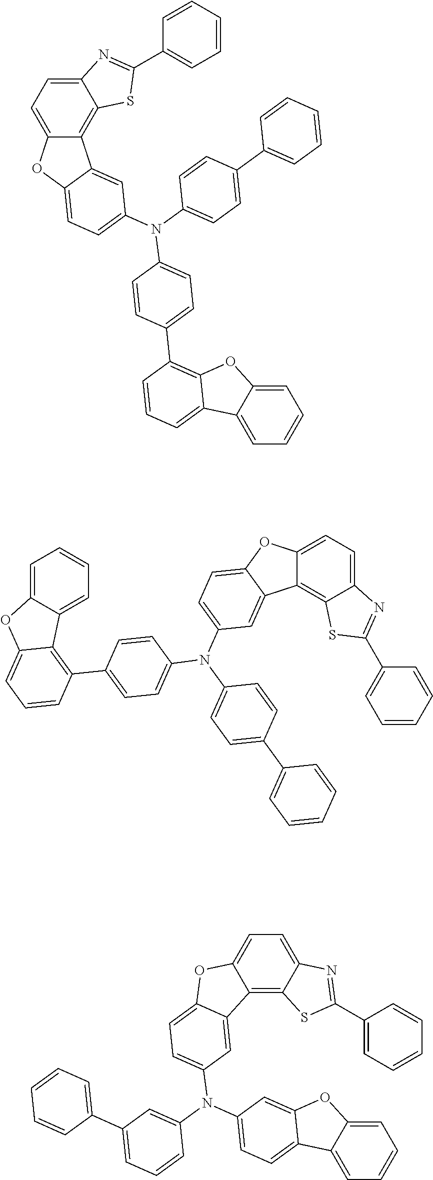 [SMILES: c1ccc(-c2ccc(N(c3ccc(-c4cccc5c4oc4ccccc45)cc3)c3ccc4oc5ccc6nc(-c7ccccc7)sc6c5c4c3)cc2)cc1 ; c1ccc(-c2ccc(N(c3ccc(-c4cccc5oc6ccccc6c45)cc3)c3ccc4oc5ccc6nc(-c7ccccc7)sc6c5c4c3)cc2)cc1 ; c1ccc(-c2cccc(N(c3ccc4c(c3)oc3ccccc34)c3ccc4oc5ccc6nc(-c7ccccc7)sc6c5c4c3)c2)cc1]

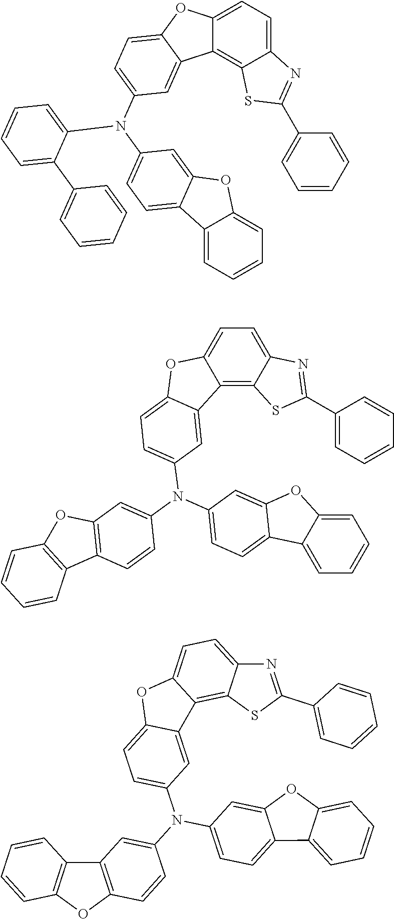 [SMILES: c1ccc(-c2nc3ccc4oc5ccc(N(c6ccc7c(c6)oc6ccccc67)c6ccccc6-c6ccccc6)cc5c4c3s2)cc1 ; c1ccc(-c2nc3ccc4oc5ccc(N(c6ccc7c(c6)oc6ccccc67)c6ccc7c(c6)oc6ccccc67)cc5c4c3s2)cc1 ; c1ccc(-c2nc3ccc4oc5ccc(N(c6ccc7c(c6)oc6ccccc67)c6ccc7oc8ccccc8c7c6)cc5c4c3s2)cc1]

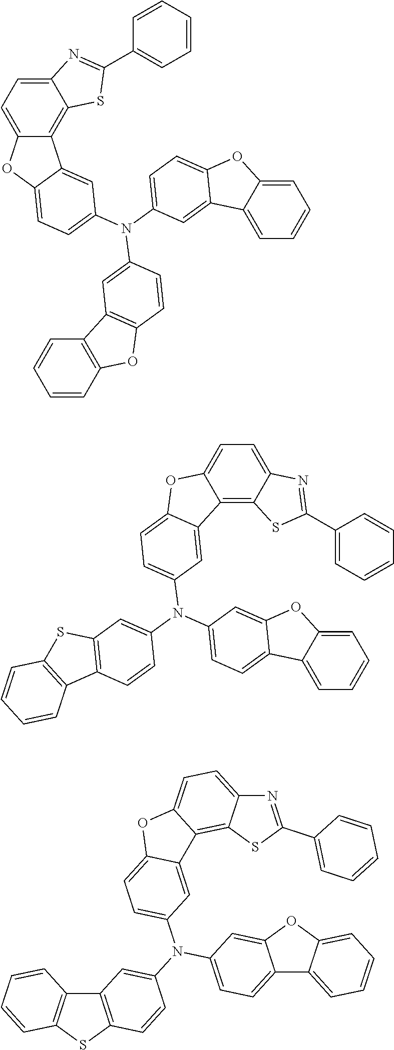 [SMILES: c1ccc(-c2nc3ccc4oc5ccc(N(c6ccc7oc8ccccc8c7c6)c6ccc7oc8ccccc8c7c6)cc5c4c3s2)cc1 ; c1ccc(-c2nc3ccc4oc5ccc(N(c6ccc7c(c6)oc6ccccc67)c6ccc7c(c6)sc6ccccc67)cc5c4c3s2)cc1 ; c1ccc(-c2nc3ccc4oc5ccc(N(c6ccc7c(c6)oc6ccccc67)c6ccc7sc8ccccc8c7c6)cc5c4c3s2)cc1]

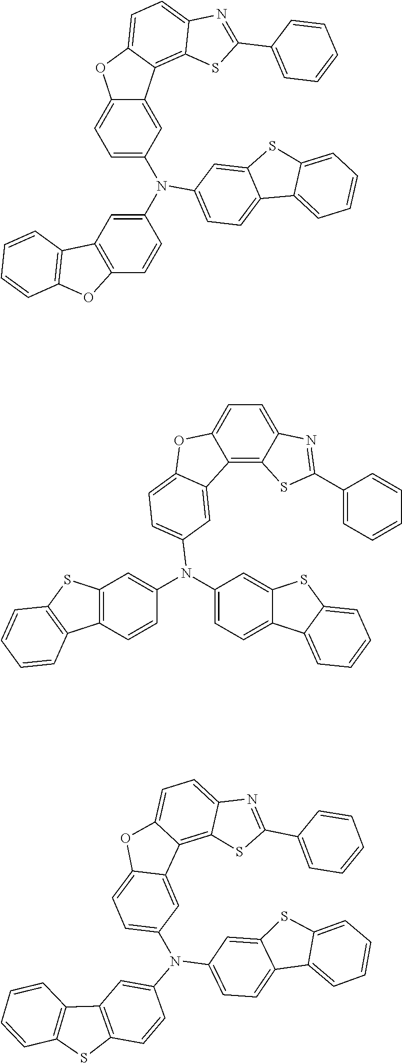 [SMILES: c1ccc(-c2nc3ccc4oc5ccc(N(c6ccc7c(c6)sc6ccccc67)c6ccc7oc8ccccc8c7c6)cc5c4c3s2)cc1 ; c1ccc(-c2nc3ccc4oc5ccc(N(c6ccc7c(c6)sc6ccccc67)c6ccc7c(c6)sc6ccccc67)cc5c4c3s2)cc1 ; c1ccc(-c2nc3ccc4oc5ccc(N(c6ccc7c(c6)sc6ccccc67)c6ccc7sc8ccccc8c7c6)cc5c4c3s2)cc1]

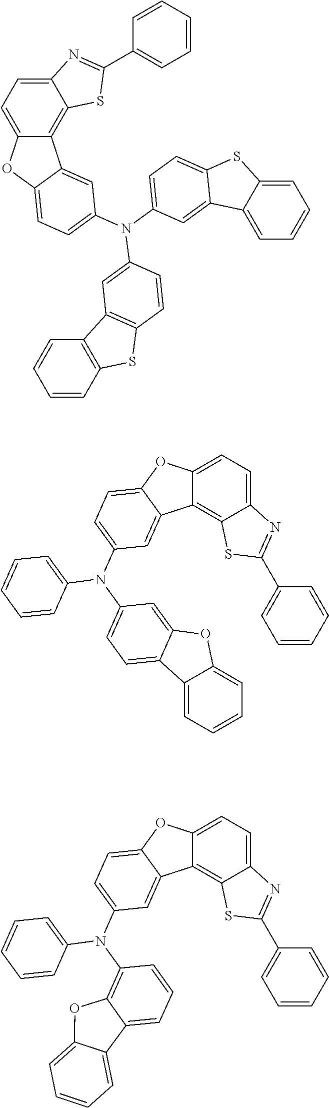 [SMILES: c1ccc(-c2nc3ccc4oc5ccc(N(c6ccc7sc8ccccc8c7c6)c6ccc7sc8ccccc8c7c6)cc5c4c3s2)cc1 ; c1ccc(-c2nc3ccc4oc5ccc(N(c6ccccc6)c6ccc7c(c6)oc6ccccc67)cc5c4c3s2)cc1 ; c1ccc(-c2nc3ccc4oc5ccc(N(c6ccccc6)c6cccc7c6oc6ccccc67)cc5c4c3s2)cc1]

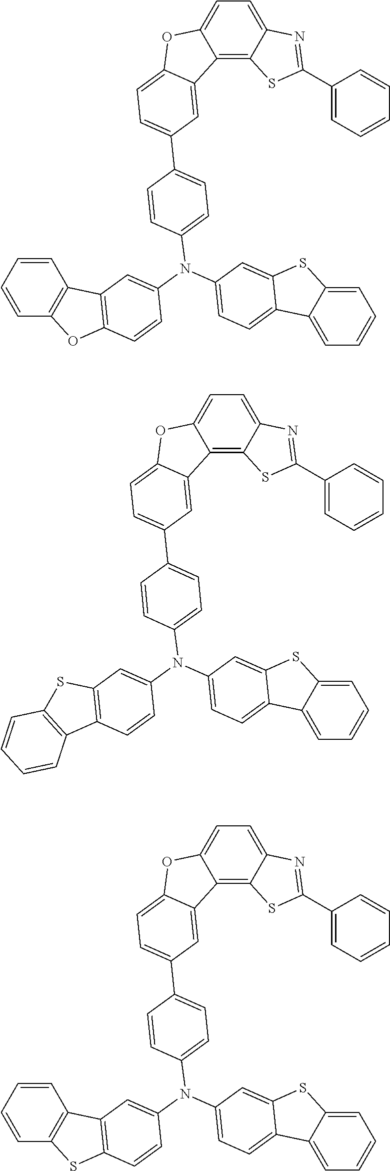 [SMILES: c1ccc(-c2nc3ccc4oc5ccc(-c6ccc(N(c7ccc8c(c7)sc7ccccc78)c7ccc8oc9ccccc9c8c7)cc6)cc5c4c3s2)cc1 ; c1ccc(-c2nc3ccc4oc5ccc(-c6ccc(N(c7ccc8c(c7)sc7ccccc78)c7ccc8c(c7)sc7ccccc78)cc6)cc5c4c3s2)cc1 ; c1ccc(-c2nc3ccc4oc5ccc(-c6ccc(N(c7ccc8c(c7)sc7ccccc78)c7ccc8sc9ccccc9c8c7)cc6)cc5c4c3s2)cc1]

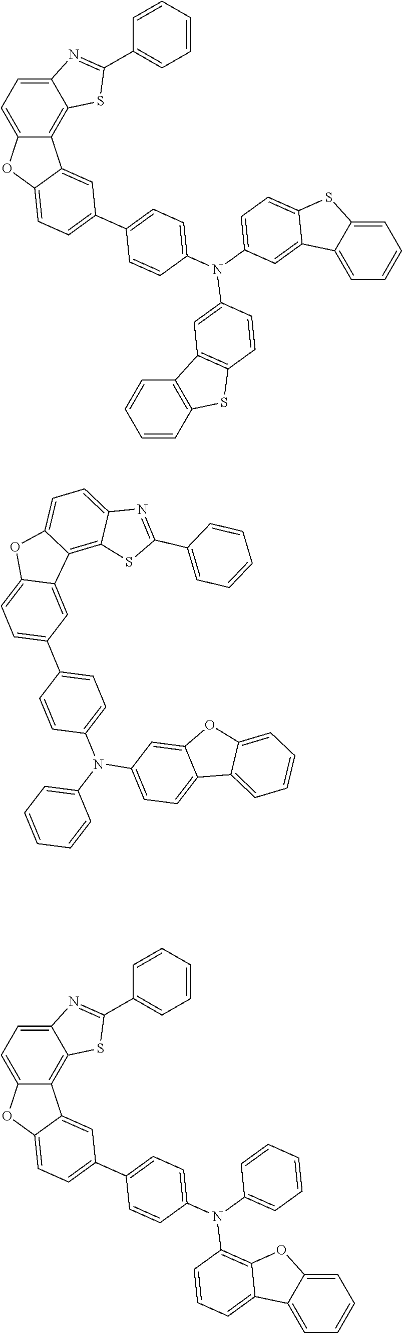 [SMILES: c1ccc(-c2nc3ccc4oc5ccc(-c6ccc(N(c7ccc8sc9ccccc9c8c7)c7ccc8sc9ccccc9c8c7)cc6)cc5c4c3s2)cc1 ; c1ccc(-c2nc3ccc4oc5ccc(-c6ccc(N(c7ccccc7)c7ccc8c(c7)oc7ccccc78)cc6)cc5c4c3s2)cc1 ; c1ccc(-c2nc3ccc4oc5ccc(-c6ccc(N(c7ccccc7)c7cccc8c7oc7ccccc78)cc6)cc5c4c3s2)cc1]

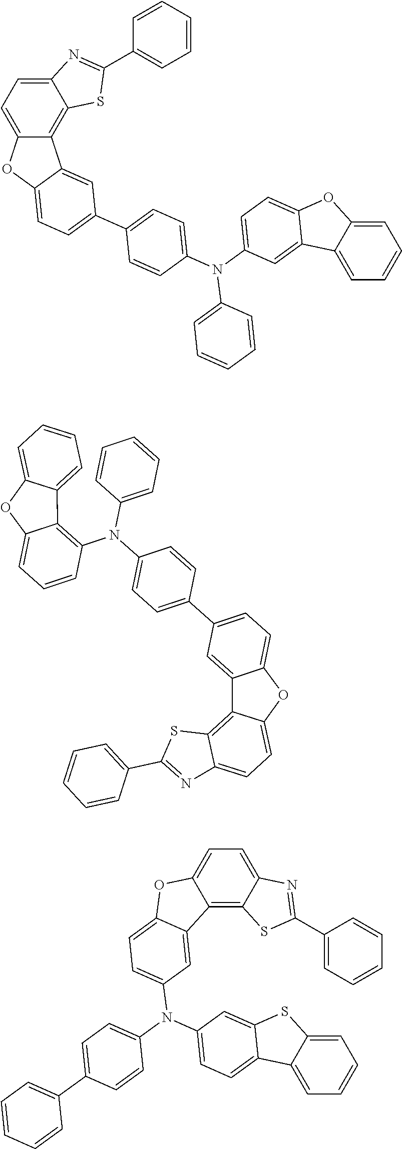 [SMILES: c1ccc(-c2nc3ccc4oc5ccc(-c6ccc(N(c7ccccc7)c7ccc8oc9ccccc9c8c7)cc6)cc5c4c3s2)cc1 ; c1ccc(-c2nc3ccc4oc5ccc(-c6ccc(N(c7ccccc7)c7cccc8oc9ccccc9c78)cc6)cc5c4c3s2)cc1 ; c1ccc(-c2ccc(N(c3ccc4c(c3)sc3ccccc34)c3ccc4oc5ccc6nc(-c7ccccc7)sc6c5c4c3)cc2)cc1]

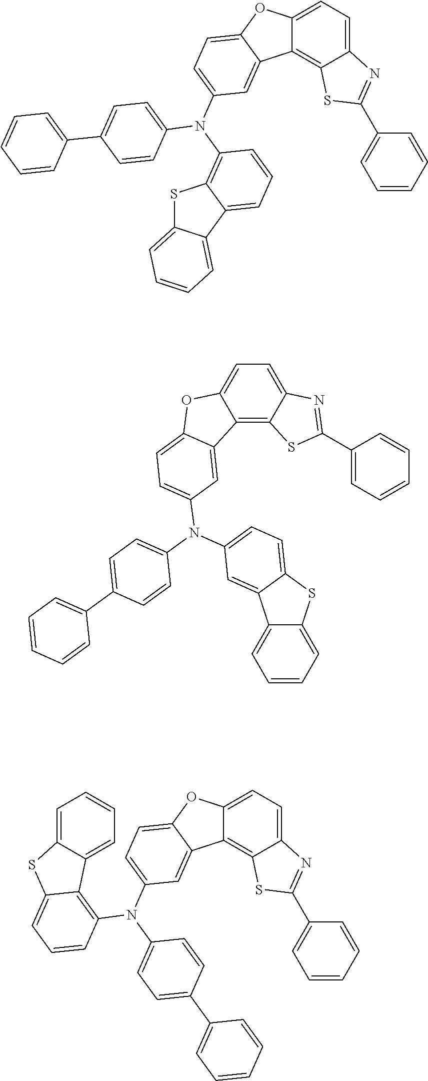 [SMILES: c1ccc(-c2ccc(N(c3ccc4oc5ccc6nc(-c7ccccc7)sc6c5c4c3)c3cccc4c3sc3ccccc34)cc2)cc1 ; c1ccc(-c2ccc(N(c3ccc4sc5ccccc5c4c3)c3ccc4oc5ccc6nc(-c7ccccc7)sc6c5c4c3)cc2)cc1 ; c1ccc(-c2ccc(N(c3ccc4oc5ccc6nc(-c7ccccc7)sc6c5c4c3)c3cccc4sc5ccccc5c34)cc2)cc1]

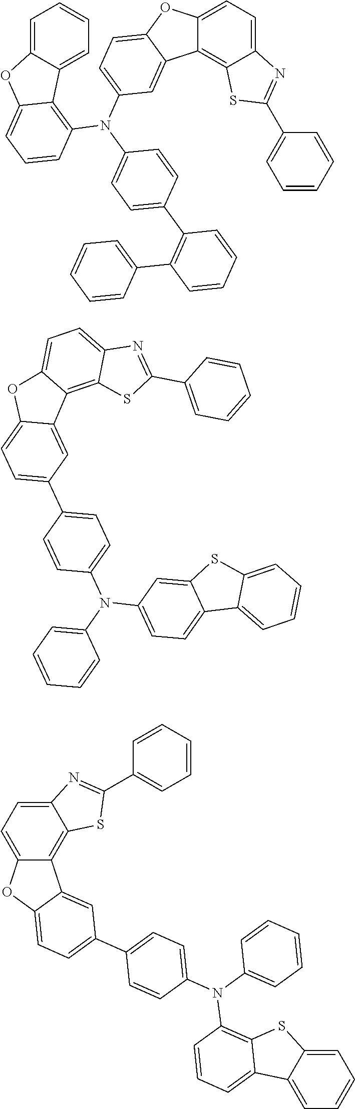

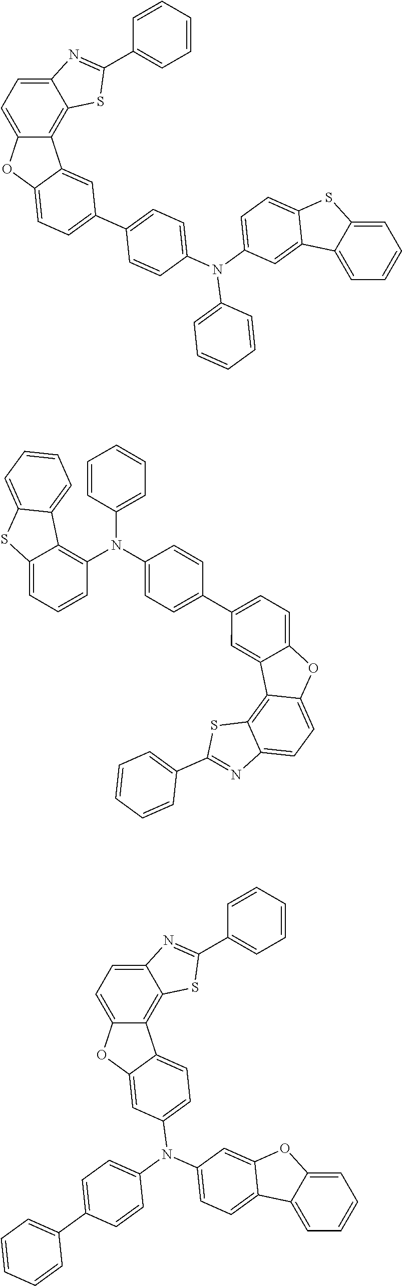 [SMILES: c1ccc(-c2nc3ccc4oc5ccc(-c6ccc(N(c7ccccc7)c7ccc8sc9ccccc9c8c7)cc6)cc5c4c3s2)cc1 ; c1ccc(-c2nc3ccc4oc5ccc(-c6ccc(N(c7ccccc7)c7cccc8sc9ccccc9c78)cc6)cc5c4c3s2)cc1 ; c1ccc(-c2ccc(N(c3ccc4c(c3)oc3ccccc34)c3ccc4c(c3)oc3ccc5nc(-c6ccccc6)sc5c34)cc2)cc1]

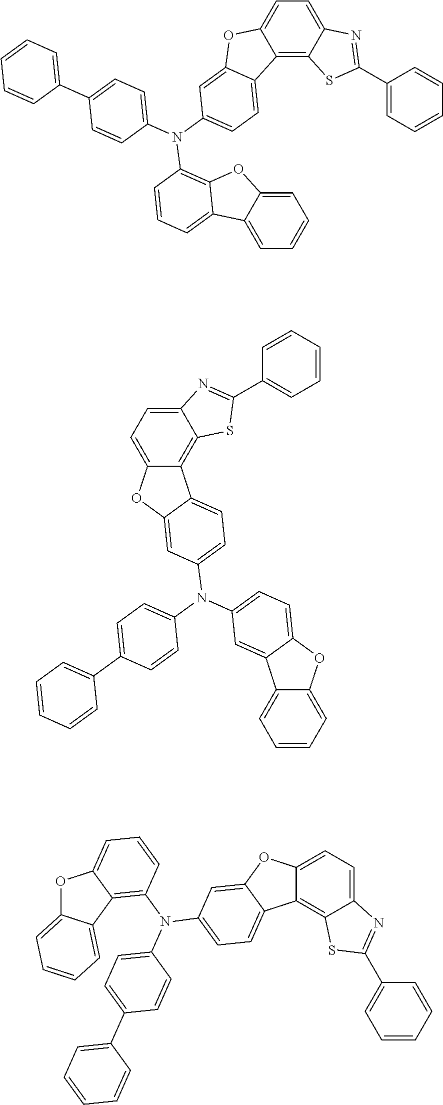 [SMILES: c1ccc(-c2ccc(N(c3ccc4c(c3)oc3ccc5nc(-c6ccccc6)sc5c34)c3cccc4c3oc3ccccc34)cc2)cc1 ; c1ccc(-c2ccc(N(c3ccc4c(c3)oc3ccc5nc(-c6ccccc6)sc5c34)c3ccc4oc5ccccc5c4c3)cc2)cc1 ; c1ccc(-c2ccc(N(c3ccc4c(c3)oc3ccc5nc(-c6ccccc6)sc5c34)c3cccc4oc5ccccc5c34)cc2)cc1]

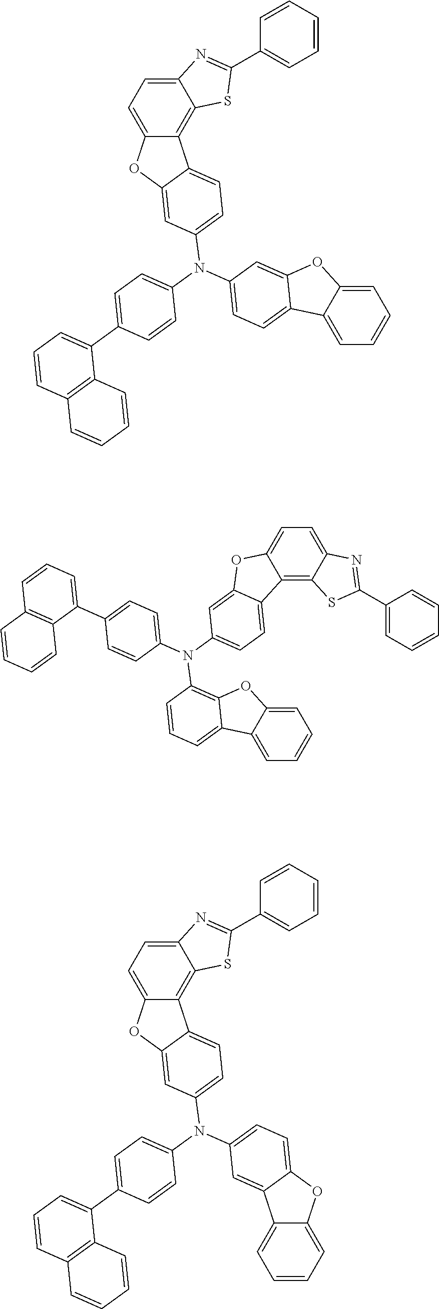 [SMILES: c1ccc(-c2nc3ccc4oc5cc(N(c6ccc(-c7cccc8ccccc78)cc6)c6ccc7c(c6)oc6ccccc67)ccc5c4c3s2)cc1 ; c1ccc(-c2nc3ccc4oc5cc(N(c6ccc(-c7cccc8ccccc78)cc6)c6cccc7c6oc6ccccc67)ccc5c4c3s2)cc1 ; c1ccc(-c2nc3ccc4oc5cc(N(c6ccc(-c7cccc8ccccc78)cc6)c6ccc7oc8ccccc8c7c6)ccc5c4c3s2)cc1]

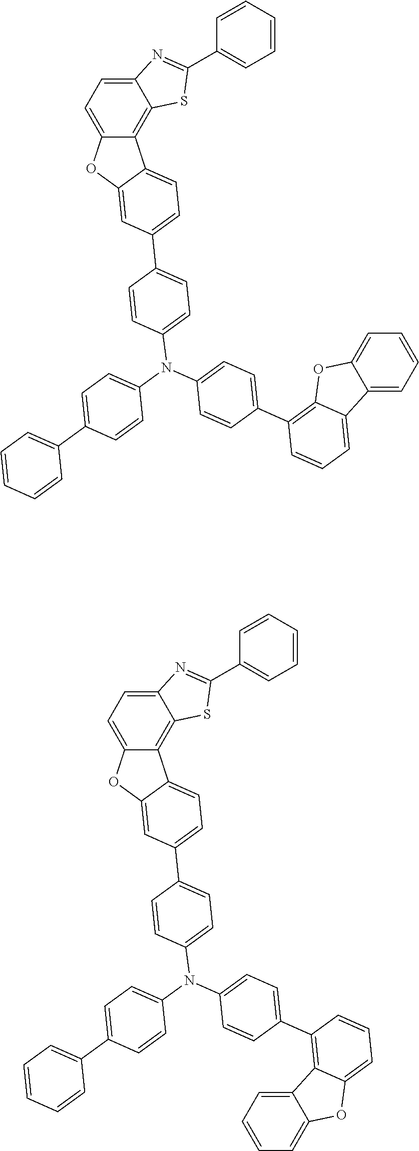 [SMILES: c1ccc(-c2ccc(N(c3ccc(-c4ccc5c(c4)oc4ccc6nc(-c7ccccc7)sc6c45)cc3)c3ccc(-c4cccc5c4oc4ccccc45)cc3)cc2)cc1 ; c1ccc(-c2ccc(N(c3ccc(-c4ccc5c(c4)oc4ccc6nc(-c7ccccc7)sc6c45)cc3)c3ccc(-c4cccc5oc6ccccc6c45)cc3)cc2)cc1]

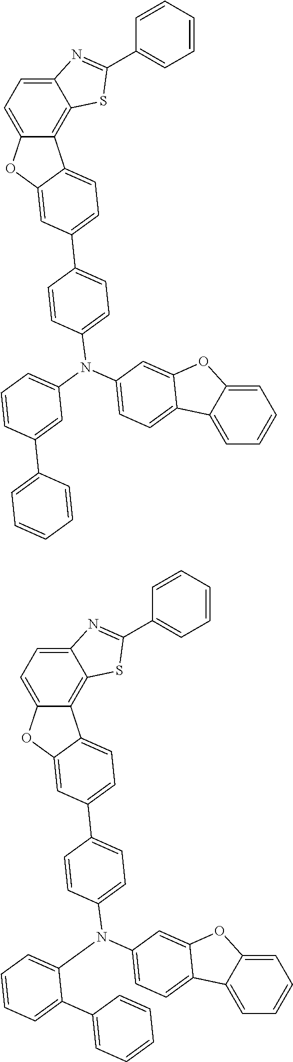 [SMILES: c1ccc(-c2cccc(N(c3ccc(-c4ccc5c(c4)oc4ccc6nc(-c7ccccc7)sc6c45)cc3)c3ccc4c(c3)oc3ccccc34)c2)cc1 ; c1ccc(-c2nc3ccc4oc5cc(-c6ccc(N(c7ccc8c(c7)oc7ccccc78)c7ccccc7-c7ccccc7)cc6)ccc5c4c3s2)cc1]

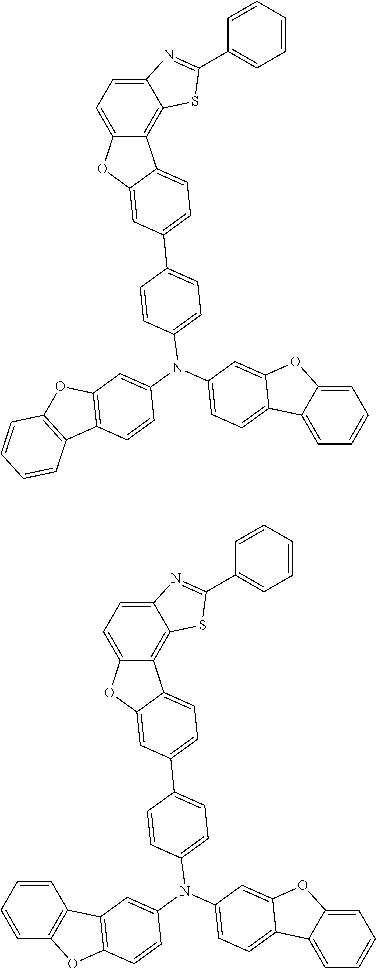 [SMILES: c1ccc(-c2nc3ccc4oc5cc(-c6ccc(N(c7ccc8c(c7)oc7ccccc78)c7ccc8c(c7)oc7ccccc78)cc6)ccc5c4c3s2)cc1 ; c1ccc(-c2nc3ccc4oc5cc(-c6ccc(N(c7ccc8c(c7)oc7ccccc78)c7ccc8oc9ccccc9c8c7)cc6)ccc5c4c3s2)cc1]

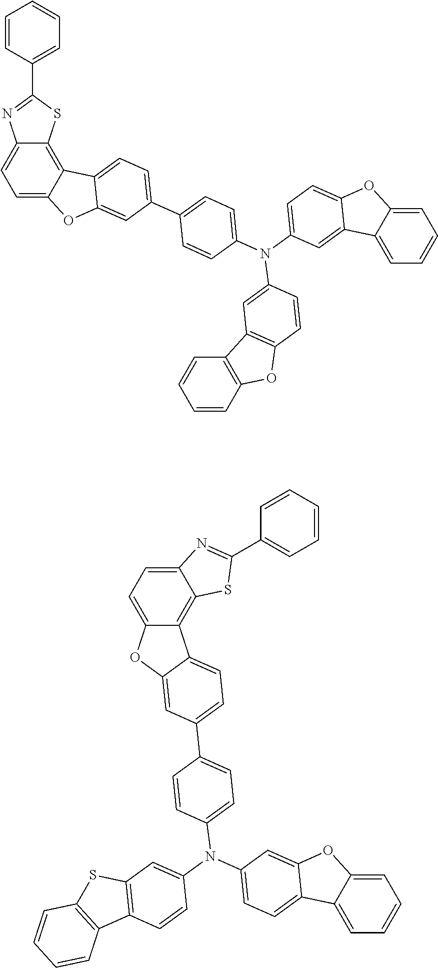 [SMILES: c1ccc(-c2nc3ccc4oc5cc(-c6ccc(N(c7ccc8oc9ccccc9c8c7)c7ccc8oc9ccccc9c8c7)cc6)ccc5c4c3s2)cc1 ; c1ccc(-c2nc3ccc4oc5cc(-c6ccc(N(c7ccc8c(c7)oc7ccccc78)c7ccc8c(c7)sc7ccccc78)cc6)ccc5c4c3s2)cc1]

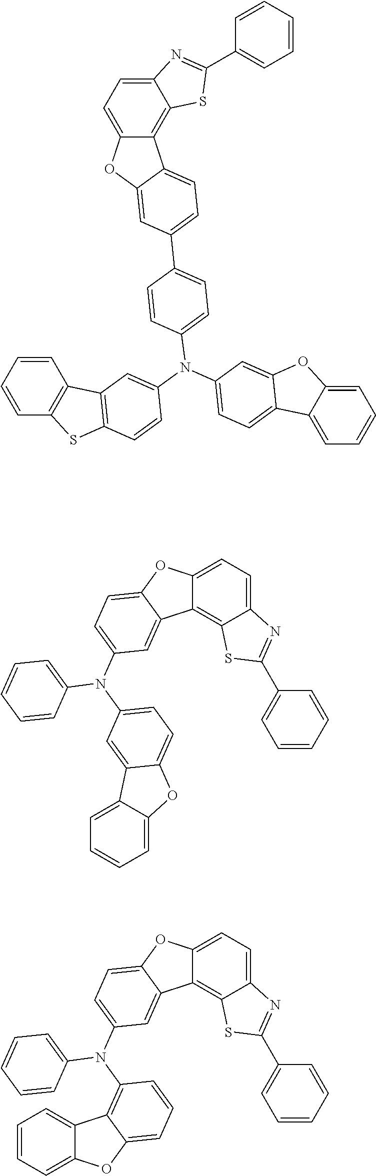 [SMILES: c1ccc(-c2nc3ccc4oc5cc(-c6ccc(N(c7ccc8c(c7)oc7ccccc78)c7ccc8sc9ccccc9c8c7)cc6)ccc5c4c3s2)cc1 ; c1ccc(-c2nc3ccc4oc5ccc(N(c6ccccc6)c6ccc7oc8ccccc8c7c6)cc5c4c3s2)cc1 ; c1ccc(-c2nc3ccc4oc5ccc(N(c6ccccc6)c6cccc7oc8ccccc8c67)cc5c4c3s2)cc1]

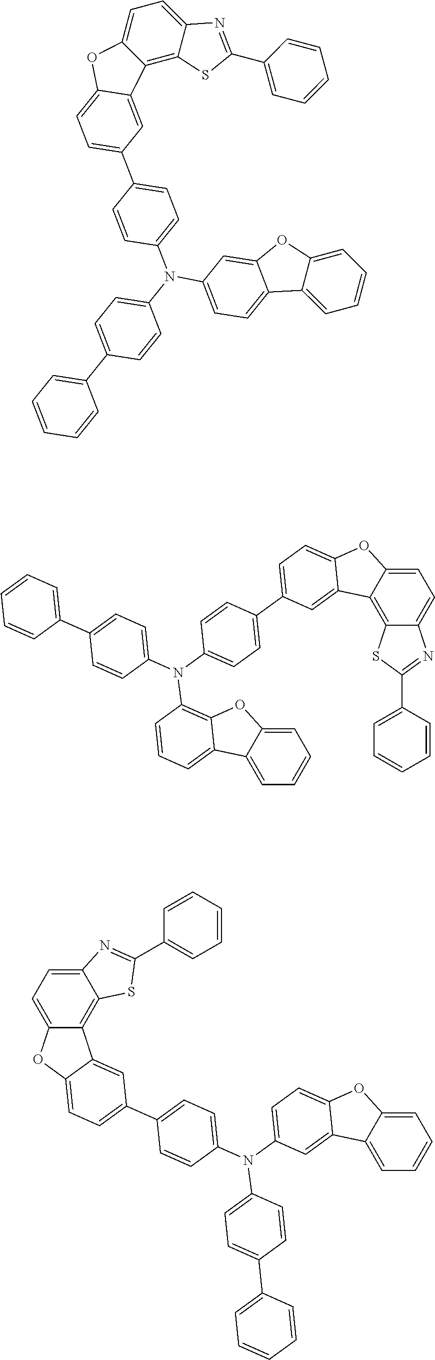 [SMILES: c1ccc(-c2ccc(N(c3ccc(-c4ccc5oc6ccc7nc(-c8ccccc8)sc7c6c5c4)cc3)c3ccc4c(c3)oc3ccccc34)cc2)cc1 ; c1ccc(-c2ccc(N(c3ccc(-c4ccc5oc6ccc7nc(-c8ccccc8)sc7c6c5c4)cc3)c3cccc4c3oc3ccccc34)cc2)cc1 ; c1ccc(-c2ccc(N(c3ccc(-c4ccc5oc6ccc7nc(-c8ccccc8)sc7c6c5c4)cc3)c3ccc4oc5ccccc5c4c3)cc2)cc1]

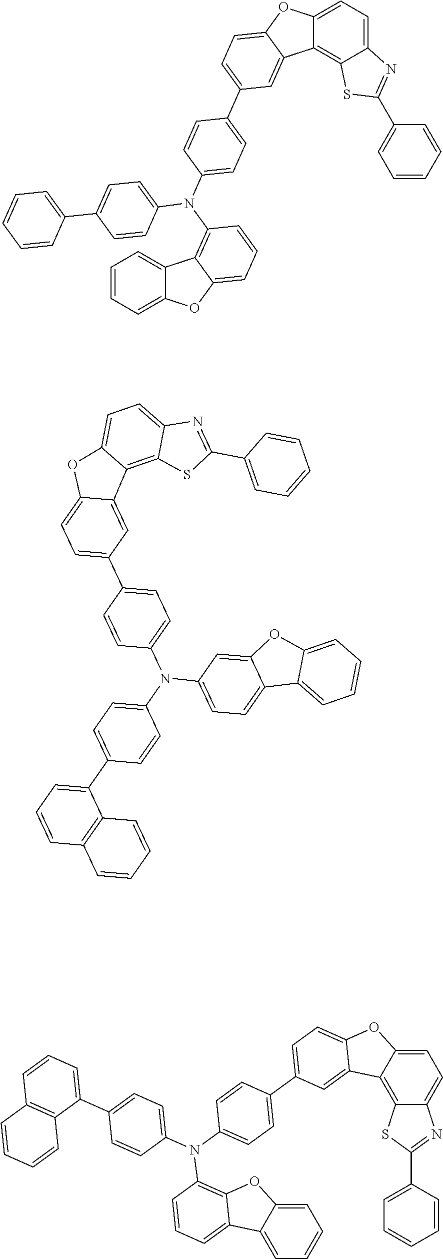 [SMILES: c1ccc(-c2ccc(N(c3ccc(-c4ccc5oc6ccc7nc(-c8ccccc8)sc7c6c5c4)cc3)c3cccc4oc5ccccc5c34)cc2)cc1 ; c1ccc(-c2nc3ccc4oc5ccc(-c6ccc(N(c7ccc(-c8cccc9ccccc89)cc7)c7ccc8c(c7)oc7ccccc78)cc6)cc5c4c3s2)cc1 ; c1ccc(-c2nc3ccc4oc5ccc(-c6ccc(N(c7ccc(-c8cccc9ccccc89)cc7)c7cccc8c7oc7ccccc78)cc6)cc5c4c3s2)cc1]

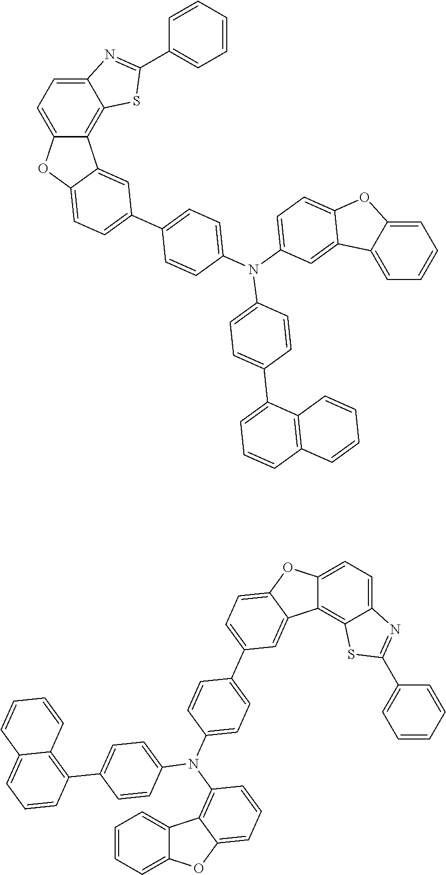 [SMILES: c1ccc(-c2nc3ccc4oc5ccc(-c6ccc(N(c7ccc(-c8cccc9ccccc89)cc7)c7ccc8oc9ccccc9c8c7)cc6)cc5c4c3s2)cc1 ; c1ccc(-c2nc3ccc4oc5ccc(-c6ccc(N(c7ccc(-c8cccc9ccccc89)cc7)c7cccc8oc9ccccc9c78)cc6)cc5c4c3s2)cc1]

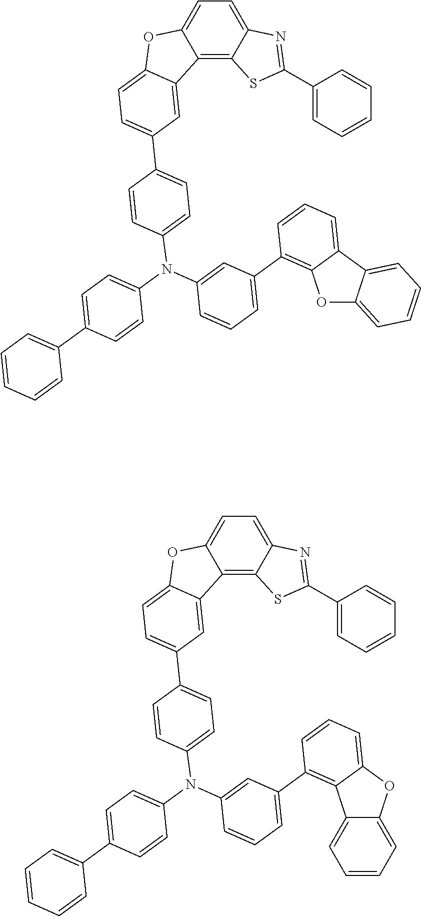 [SMILES: c1ccc(-c2ccc(N(c3ccc(-c4ccc5oc6ccc7nc(-c8ccccc8)sc7c6c5c4)cc3)c3cccc(-c4cccc5c4oc4ccccc45)c3)cc2)cc1 ; c1ccc(-c2ccc(N(c3ccc(-c4ccc5oc6ccc7nc(-c8ccccc8)sc7c6c5c4)cc3)c3cccc(-c4cccc5oc6ccccc6c45)c3)cc2)cc1]

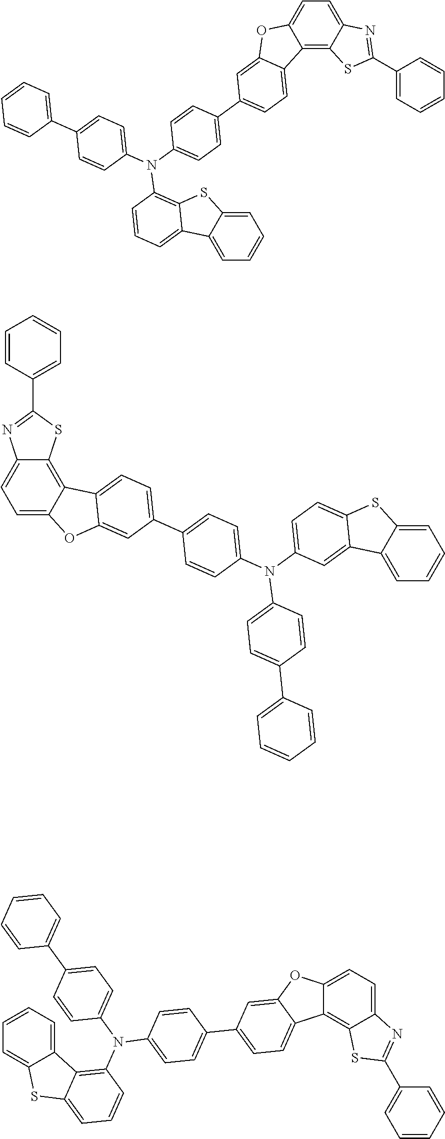 [SMILES: c1ccc(-c2ccc(N(c3ccc(-c4ccc5c(c4)oc4ccc6nc(-c7ccccc7)sc6c45)cc3)c3cccc4c3sc3ccccc34)cc2)cc1 ; c1ccc(-c2ccc(N(c3ccc(-c4ccc5c(c4)oc4ccc6nc(-c7ccccc7)sc6c45)cc3)c3ccc4sc5ccccc5c4c3)cc2)cc1 ; c1ccc(-c2ccc(N(c3ccc(-c4ccc5c(c4)oc4ccc6nc(-c7ccccc7)sc6c45)cc3)c3cccc4sc5ccccc5c34)cc2)cc1]

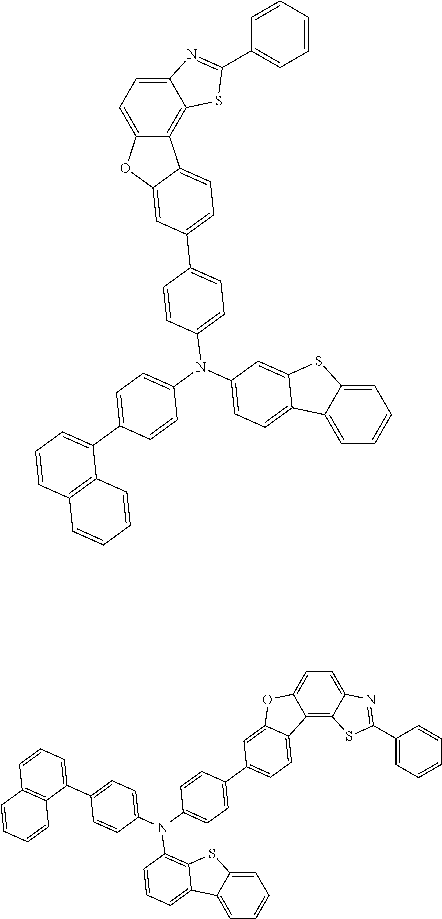 [SMILES: c1ccc(-c2nc3ccc4oc5cc(-c6ccc(N(c7ccc(-c8cccc9ccccc89)cc7)c7ccc8c(c7)sc7ccccc78)cc6)ccc5c4c3s2)cc1 ; c1ccc(-c2nc3ccc4oc5cc(-c6ccc(N(c7ccc(-c8cccc9ccccc89)cc7)c7cccc8c7sc7ccccc78)cc6)ccc5c4c3s2)cc1]

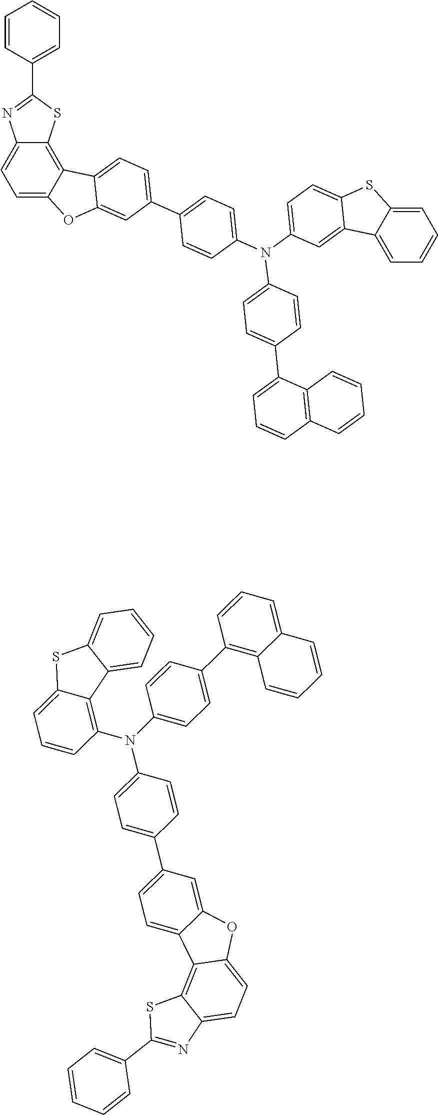 [SMILES: c1ccc(-c2nc3ccc4oc5cc(-c6ccc(N(c7ccc(-c8cccc9ccccc89)cc7)c7ccc8sc9ccccc9c8c7)cc6)ccc5c4c3s2)cc1 ; c1ccc(-c2nc3ccc4oc5cc(-c6ccc(N(c7ccc(-c8cccc9ccccc89)cc7)c7cccc8sc9ccccc9c78)cc6)ccc5c4c3s2)cc1]

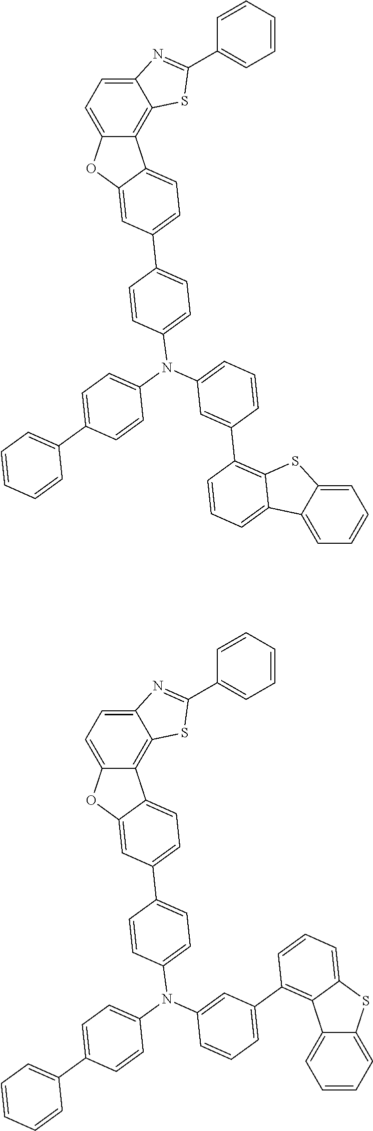 [SMILES: c1ccc(-c2ccc(N(c3ccc(-c4ccc5c(c4)oc4ccc6nc(-c7ccccc7)sc6c45)cc3)c3cccc(-c4cccc5c4sc4ccccc45)c3)cc2)cc1 ; c1ccc(-c2ccc(N(c3ccc(-c4ccc5c(c4)oc4ccc6nc(-c7ccccc7)sc6c45)cc3)c3cccc(-c4cccc5sc6ccccc6c45)c3)cc2)cc1]

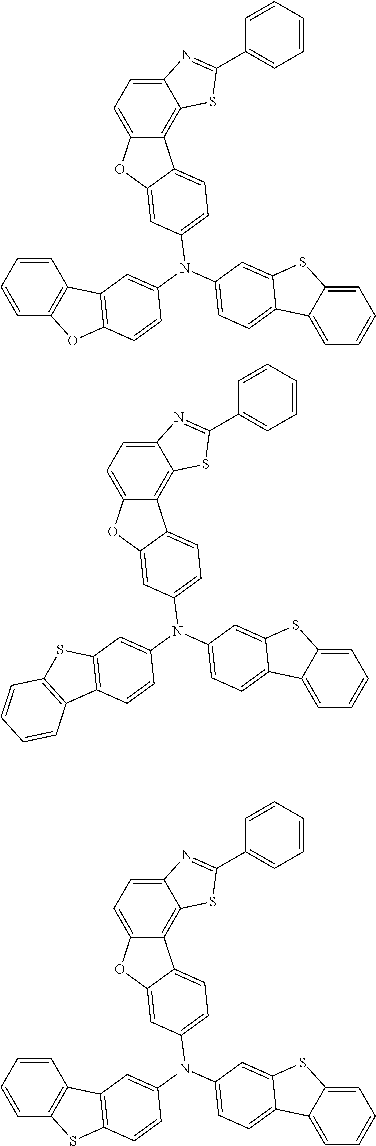

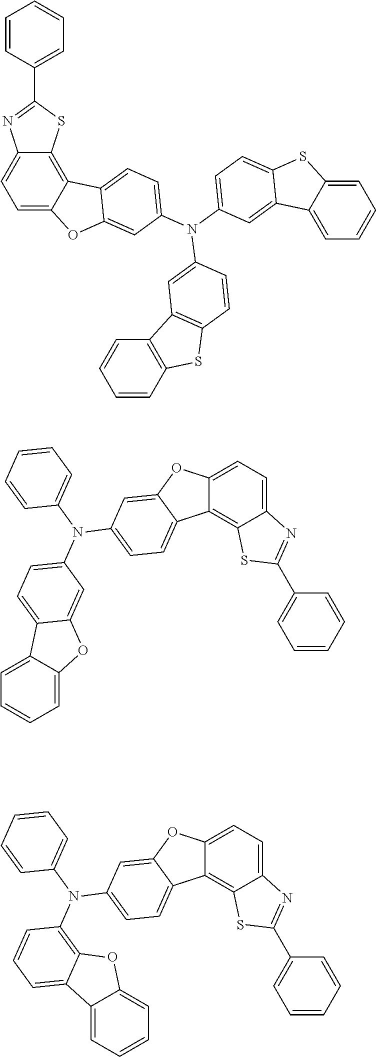 [SMILES: c1ccc(-c2nc3ccc4oc5cc(N(c6ccc7sc8ccccc8c7c6)c6ccc7sc8ccccc8c7c6)ccc5c4c3s2)cc1 ; c1ccc(-c2nc3ccc4oc5cc(N(c6ccccc6)c6ccc7c(c6)oc6ccccc67)ccc5c4c3s2)cc1 ; c1ccc(-c2nc3ccc4oc5cc(N(c6ccccc6)c6cccc7c6oc6ccccc67)ccc5c4c3s2)cc1]

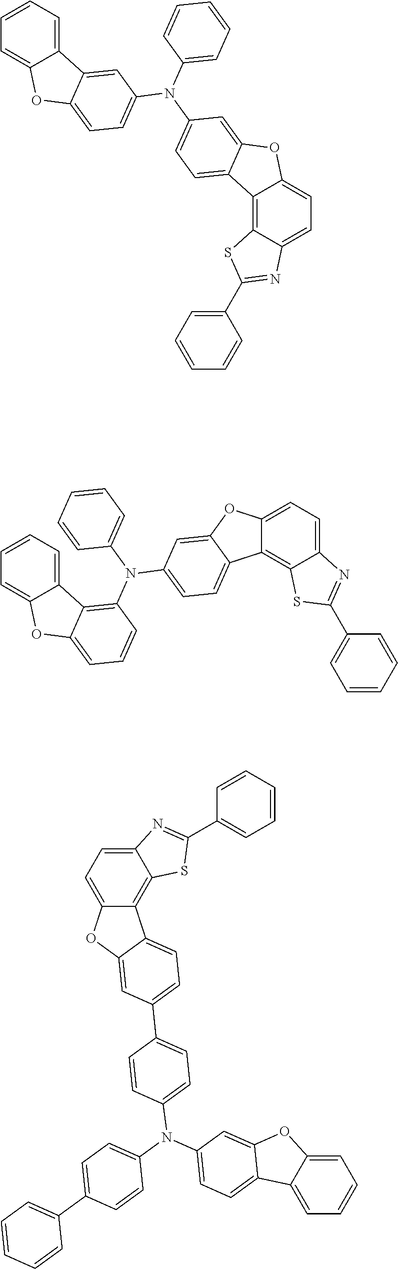 [SMILES: c1ccc(-c2nc3ccc4oc5cc(N(c6ccccc6)c6ccc7oc8ccccc8c7c6)ccc5c4c3s2)cc1 ; c1ccc(-c2nc3ccc4oc5cc(N(c6ccccc6)c6cccc7oc8ccccc8c67)ccc5c4c3s2)cc1 ; c1ccc(-c2ccc(N(c3ccc(-c4ccc5c(c4)oc4ccc6nc(-c7ccccc7)sc6c45)cc3)c3ccc4c(c3)oc3ccccc34)cc2)cc1]

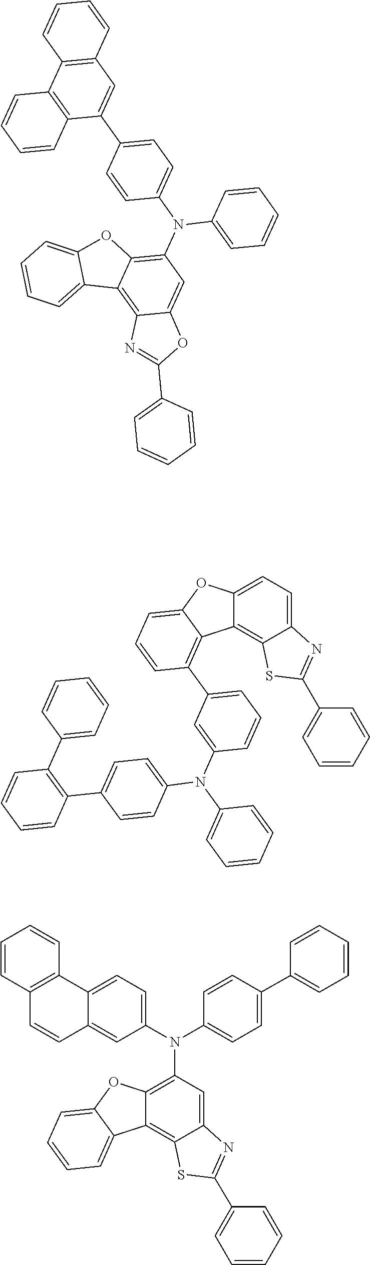 [SMILES: c1ccc(-c2nc3c(cc(N(c4ccccc4)c4ccc(-c5cc6ccccc6c6ccccc56)cc4)c4oc5ccccc5c43)o2)cc1 ; c1ccc(-c2nc3ccc4oc5cccc(-c6cccc(N(c7ccccc7)c7ccc(-c8ccccc8-c8ccccc8)cc7)c6)c5c4c3s2)cc1 ; c1ccc(-c2ccc(N(c3ccc4c(ccc5ccccc54)c3)c3cc4nc(-c5ccccc5)sc4c4c3oc3ccccc34)cc2)cc1]

The compound in which Li is a single bond and R₁ is

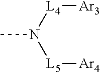

in Chemical Formula 1 can be prepared by a preparation method as shown in Reaction Scheme 1 below. Some compounds can be prepared by a preparation method as shown in Reaction Scheme 2 below, and other compounds can be prepared similarly.

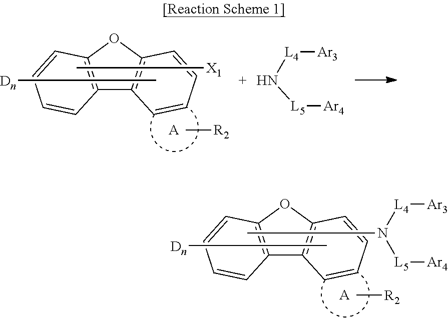

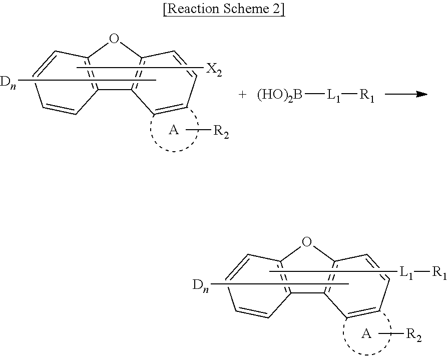

In the Reaction Schemes 1 and 2, R₁, R₂, L₁, L₄, L₅, Ar₃, Ar₄, A, D, and n are as defined in the above Chemical Formula 1, and X₁ and X₂ are each independently halogen, and preferably chloro or bromo.

The Reaction Scheme 1 is an amine substitution reaction, and preferably performed in the presence of a palladium catalyst and a base, and the reactor for the amine substitution reaction can be changed as known in the art. In addition, the Reaction Scheme 2 is a Suzuki coupling reaction, and preferably performed in the presence of a palladium catalyst and a base, and the reactor for the Suzuki coupling reaction can be changed as known in the art. The preparation method can be more specifically described in the Preparation Examples described below.

In addition, provided is an organic light emitting device including the above-mentioned compound of Chemical Formula 1. As an example, provided is an organic light emitting device including: a first electrode; a second electrode that is provided opposite to the first electrode; and one or more organic material layers that are provided between the first electrode and the second electrode, wherein at least one layer of the organic material layers includes at least one compound of Chemical Formula 1.

The organic material layer of the organic light emitting device of the present disclosure can have a single-layer structure, or it can have a multilayered structure in which two or more organic material layers are stacked. For example, the organic light emitting device of the present disclosure can have a structure including a hole injection layer, a hole transport layer, a light emitting layer, an electron transport layer, an electron injection layer, and the like as the organic material layer. However, the structure of the organic light emitting device is not limited thereto, and it can include a smaller number of organic material layers.

In addition, the organic material layer can include an electron blocking layer or a light emitting layer, and the electron blocking layer or the light emitting layer can include the compound of Chemical Formula 1.

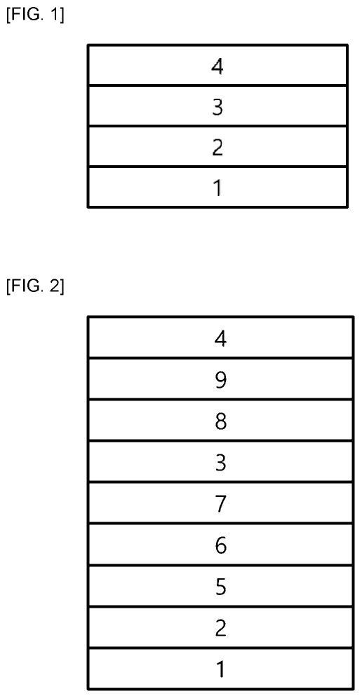

Further, the organic light emitting device according to the present disclosure can be a normal type organic light emitting device in which an anode, one or more organic material layers and a cathode are sequentially stacked on a substrate. Further, the organic light emitting device according to the present disclosure can be an inverted type organic light emitting device in which a cathode, one or more organic material layers and an anode are sequentially stacked on a substrate. For example, the structure of an organic light emitting device according to an embodiment of the present disclosure is illustrated in FIGS. 1 and 2.

FIG. 1 shows an example of an organic light emitting device including a substrate 1, an anode 2, a light emitting layer 3, and a cathode 4. FIG. 2 shows an example of an organic light emitting device including a substrate 1, an anode 2, a hole injection layer 5, a hole transport layer 6, an electron blocking layer 7, a light emitting layer 3, a hole blocking layer 8, an electron injection and transport layer 9, and a cathode 4. In such a structure, the compound of Chemical Formula 1 can be included in the light emitting layer or in the electron blocking layer.

The organic light emitting device according to the present disclosure can be manufactured by materials and methods known in the art, except that one or more layers of the organic material layers includes the compound of Chemical Formula 1. Moreover, when the organic light emitting device includes a plurality of organic material layers, the organic material layers can be formed of the same material or different materials.

For example, the organic light emitting device according to the present disclosure can be manufactured by sequentially stacking a first electrode, an organic material layer and a second electrode on a substrate. In this case, the organic light emitting device can be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound of Chemical Formula 1 can be formed into an organic layer by a solution coating method as well as a vacuum deposition method at the time of manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device can be manufactured by sequentially depositing a cathode material, an organic material layer and an anode material on a substrate (International Publication WO2003/012890). However, the preparation method is not limited thereto.

As an example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof, metal oxides such as zinc oxides, indium oxides, indium tin oxides (ITO), and indium zinc oxides (IZO); a combination of metals and oxides, such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole-injecting effect to the light emitting layer or the light emitting material, prevents excitons produced in the light emitting layer from moving to an electron injection layer or the electron injection material, and is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which can receive holes from the anode or the hole injection layer and transfer the holes to the light emitting layer. Specific examples thereof include an arylamine-based organic material, a conductive polymer, a block copolymer in which a conjugate portion and a non-conjugate portion are present together, and the like, but are not limited thereto.

The electron blocking layer is a layer placed between the hole transport layer and the light emitting layer to prevent electrons injected from the cathode from being transferred to the hole transport layer without recombination in the light emitting layer, and is also called an electron suppressing layer. A material having the electron affinity lower than that of the electron transport layer is preferable for the electron blocking layer. Preferably, the material of Chemical Formula 1 of the present disclosure can be used as the electron blocking material.

The light emitting material is suitably a material capable of emitting light in a visible ray region by receiving holes and electrons from the hole transport layer and the electron transport layer, respectively, to combine them, and having good quantum efficiency to fluorescence or phosphorescence. Specific examples thereof include 8-hydroxy-quinoline aluminum complex (Alq₃); a carbazole-based compound; a dimerized styryl compound; BAlq; a 10-hydroxybenzo quinoline-metal compound; a benzoxazole-, benzothiazole- and benzimidazole-based compound; a poly(p-phenylenevinylene) (PPV)-based polymer; a spiro compound; polyfluorene, rubrene, and the like, but are not limited thereto.

Specifically, the light emitting layer can include a host material and a dopant material. The host material can be a fused aromatic ring derivative, a heterocycle-containing compound or the like. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto. Preferably, the material of Chemical Formula 1 of the present disclosure can be used as the host material, and one or more materials of Chemical Formula 1 can be included as the host material. Preferably, when two types of the compound of Chemical Formula 1 are used in the light emitting layer, a weight ratio thereof is 10:90 to 90:10, and more preferably 20:80 to 80:20, 30:70 to 70:30 or 40:60 to 60:40.

The dopant material includes an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

For example, at least one selected from the group consisting of the following can be used as the dopant material, but the present disclosure is not limited thereto:

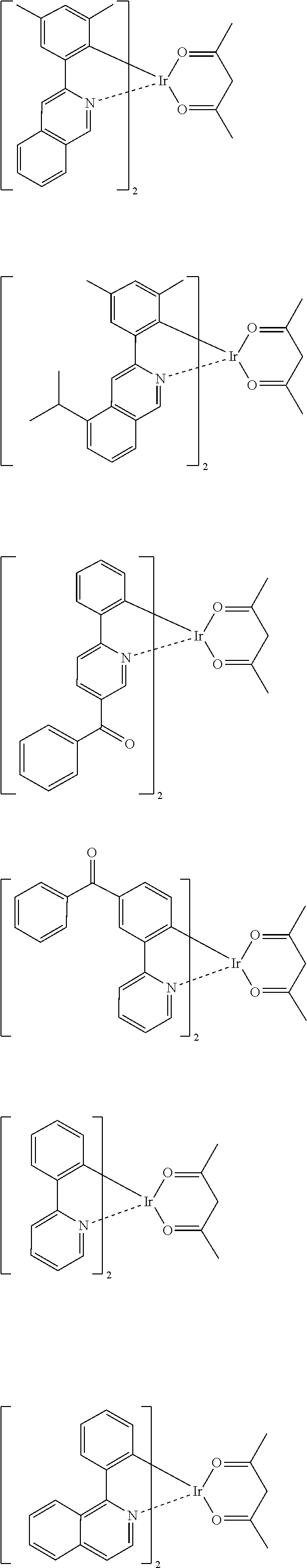

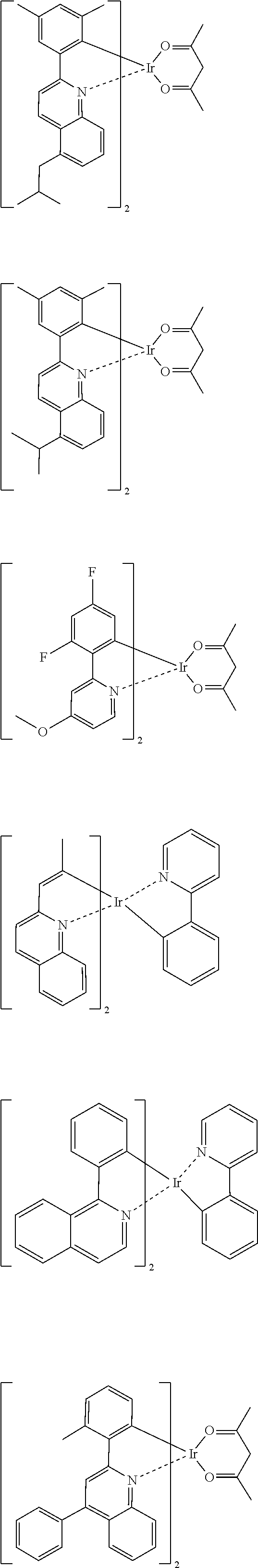

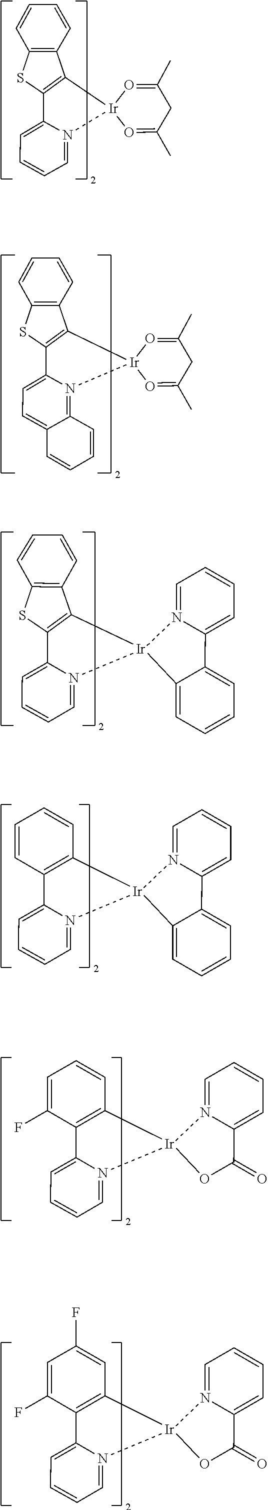

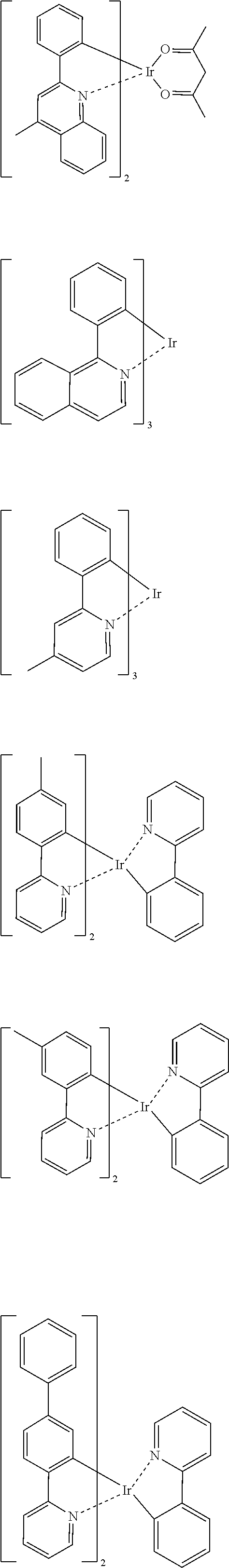

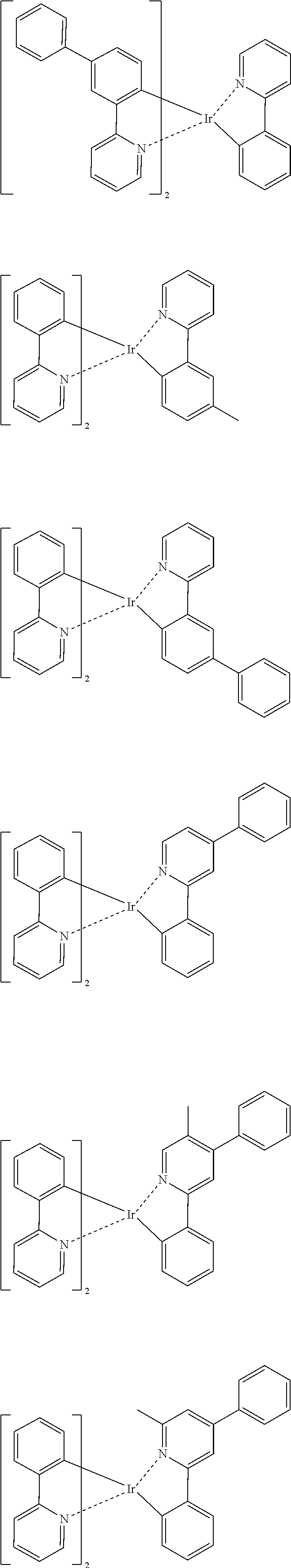

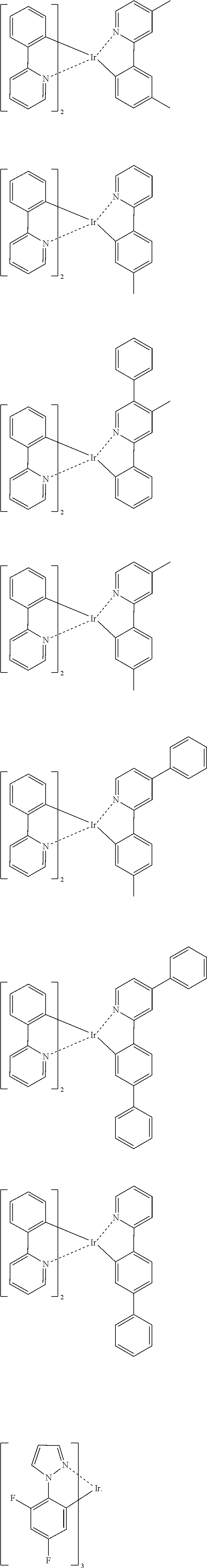

The hole blocking layer is a layer placed between the electron transport layer and the light emitting layer to prevent holes injected from the anode from being transferred to the electron transport layer without recombination in the light emitting layer, is also called a hole suppressing layer. A material having high ionization energy is preferable for the hole blocking layer.

The electron transport layer is a layer which receives electrons from an electron injection layer and transports the electrons to a light emitting layer. As an electron transport material, a material which can receive electrons well from a cathode and transfer the electrons to a light emitting layer and has large mobility for electrons is suitable. Examples thereof include an Al complex of 8-hydroxyquinoline, a complex including Alq₃, an organic radical compound, a hydroxyflavone-metal complex, and the like, but are not limited thereto. The electron transport layer can be used with any desired cathode material, as used according to the related art. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples thereof include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)-beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

Meanwhile, the “electron injection and transport layer” used herein is a layer that performs both the roles of the electron injection layer and the electron transport layer, and the material for each layer can be used alone or in combination, but the present disclosure is not limited thereto.

The organic light emitting device according to the present disclosure can be a bottom emission device, a top emission device, or a double-sided emission device, and in particular, can be a bottom emission device requiring relatively high luminous efficiency.

In addition, the compound of Chemical Formula 1 can be included in an organic solar cell or an organic transistor in addition to an organic light emitting device.

The preparation of the compound of Chemical Formula 1 and the organic light emitting device containing the same will be described in detail in the following examples. However, these examples are presented for illustrative purposes only, and are not intended to limit the scope of the present disclosure.

### PREPARATION EXAMPLES

### Preparation Example 1-1

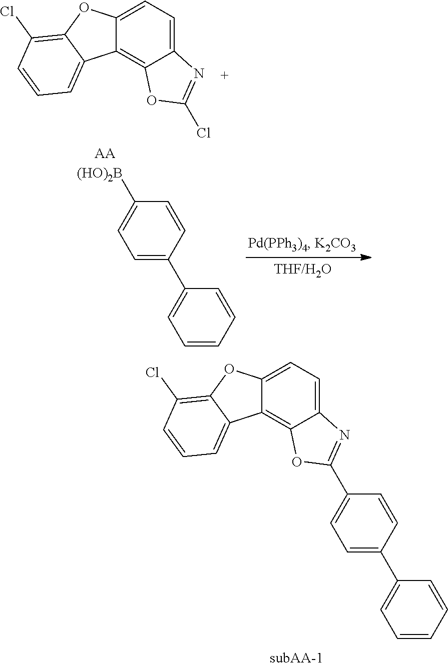

Compound AA (15 g, 53.9 mmol) and [1,1′-biphenyl]-4-ylboronic acid (10.7 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.4 g of Compound subAA-1 (yield 63%, MS: [M+H]+=396).

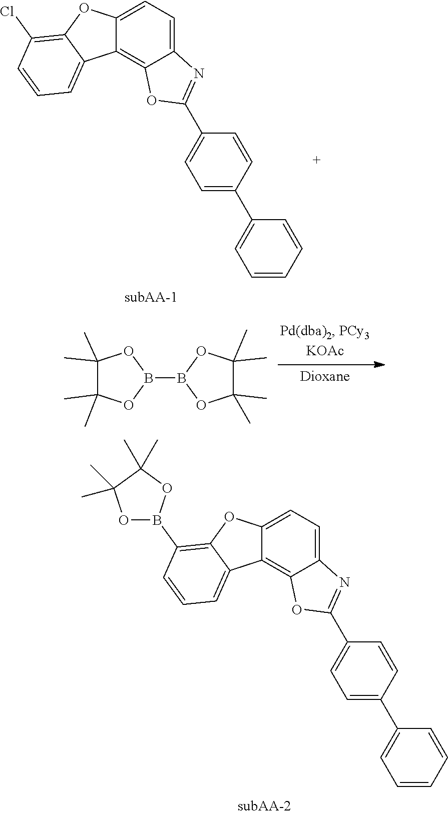

Compound subAA-1 (15 g, 37.9 mmol) and bis(pinacolato)diboron (10.6 g, 41.7 mmol) were added to 300 ml of 1,4-dioxane under a nitrogen atmosphere, and the mixture was stirred and refluxed. Then, potassium acetate (5.6 g, 56.8 mmol) was added and stirred sufficiently, followed by adding bis(dibenzylideneacetone)palladium(0) (0.7 g, 1.1 mmol) and tricyclohexylphosphine (0.6 g, 2.3 mmol). After 8 hours of reaction, cooling was performed to room temperature. Then, the organic layer was separated using chloroform and water, and distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.4 g of Compound subAA-2 (yield 67%, MS: [M+H]+=488).

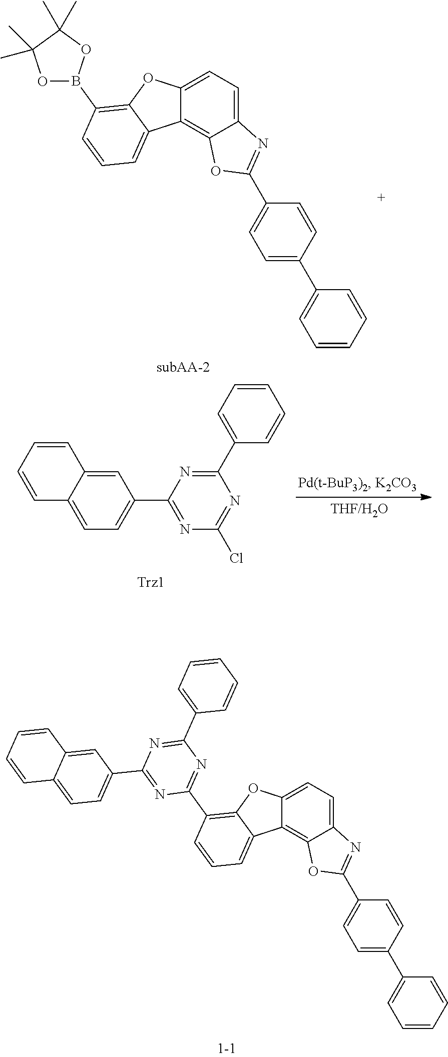 [SMILES: CC1(C)OB(c2cccc3c2oc2ccc4nc(-c5ccc(-c6ccccc6)cc5)oc4c23)OC1(C)C ; Clc1nc(-c2ccccc2)nc(-c2ccc3ccccc3c2)n1 ; c1ccc(-c2ccc(-c3nc4ccc5oc6c(-c7nc(-c8ccccc8)nc(-c8ccc9ccccc9c8)n7)cccc6c5c4o3)cc2)cc1]

Compound subAA-2 (15 g, 30.8 mmol) and Trz1 (9.8 g, 30.8 mmol) were added to 300 ml of THF under a nitrogen atmosphere, and the mixture was stirred and refluxed. Then, potassium carbonate (12.8 g, 92.3 mmol) was dissolved in 38 ml of water, and then added thereto. Thereafter, it was stirred sufficiently, followed by adding bis(tri-tert-butylphosphine)palladium(0) (0.2 g, 0.3 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.9 g of Compound 1-1 (yield 60%, MS: [M+H]+=643).

### Preparation Example 1-2

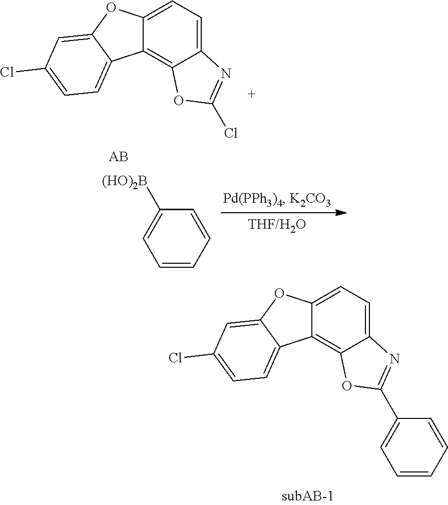 [SMILES: Clc1ccc2c(c1)oc1ccc3nc(Cl)oc3c12 ; OB(O)c1ccccc1 ; Clc1ccc2c(c1)oc1ccc3nc(-c4ccccc4)oc3c12]

Compound AB (15 g, 53.9 mmol) and phenylboronic acid (6.6 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)-palladium(0) (0.6 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.4 g of Compound subAB-1 (yield 78%, MS: [M+H]+=320).

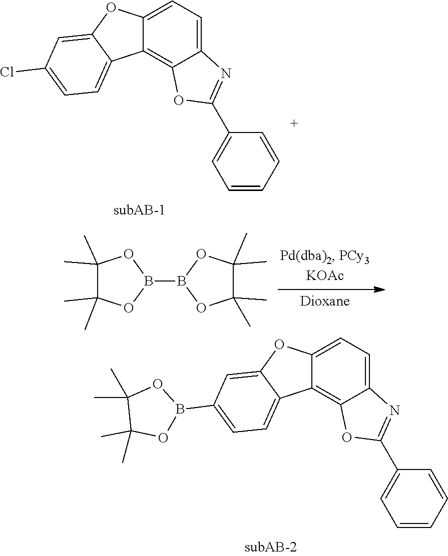

Compound subAB-1 (15 g, 46.9 mmol) and bis(pinacolato)diboron (13.1 g, 51.6 mmol) were added to 300 ml of 1,4-dioxane under a nitrogen atmosphere, and the mixture was stirred and refluxed. Then, potassium acetate (6.9 g, 70.4 mmol) was added and stirred sufficiently, followed by adding bis(dibenzylideneacetone)palladium(0) (0.8 g, 1.4 mmol) and tricyclohexylphosphine (0.8 g, 2.8 mmol). After 9 hours of reaction, cooling was performed to room temperature. Then, the organic layer was separated using chloroform and water, and distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.3 g of Compound subAB-2 (yield 74%, MS: [M+H]+=412).

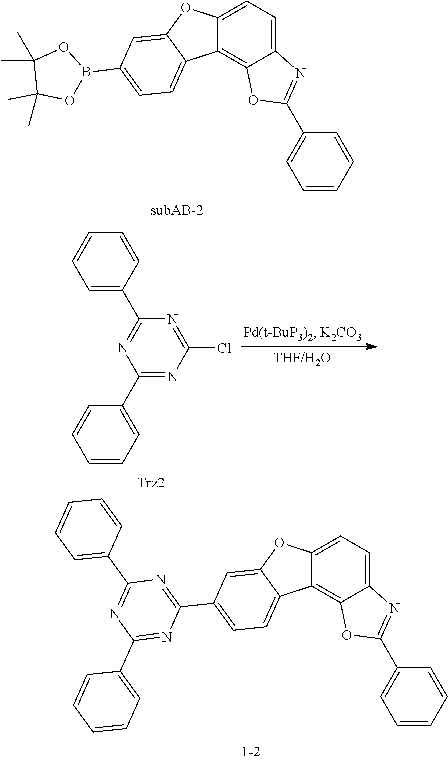

Compound subAB-2 (15 g, 36.5 mmol) and Trz2 (9.8 g, 36.5 mmol) were added to 300 ml of THF under a nitrogen atmosphere, and the mixture was stirred and refluxed. Then, potassium carbonate (15.1 g, 109.4 mmol) was dissolved in 45 ml of water, and then added thereto. Thereafter, it was stirred sufficiently, followed by adding bis(tri-tert-butylphosphine)palladium(0) (0.2 g, 0.4 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.4 g of Compound 1-2 (yield 66%, MS: [M+H]+=517).

### Preparation Example 1-3

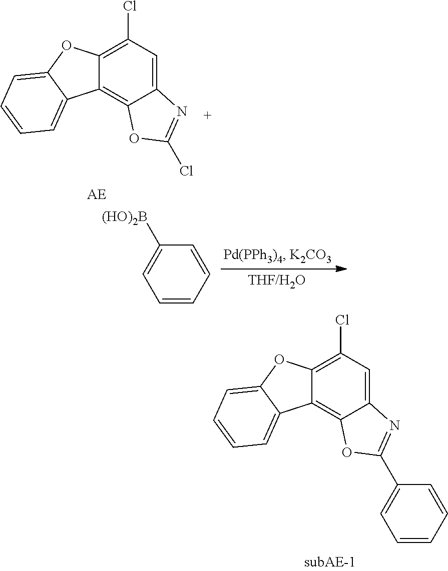 [SMILES: Clc1nc2cc(Cl)c3oc4ccccc4c3c2o1 ; OB(O)c1ccccc1 ; Clc1cc2nc(-c3ccccc3)oc2c2c1oc1ccccc12]

Compound AE (15 g, 53.9 mmol) and phenylboronic acid (6.6 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)-palladium(0) (0.6 g, 0.5 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.7 g of Compound subAE-1 (yield 62%, MS: [M+H]+=320).

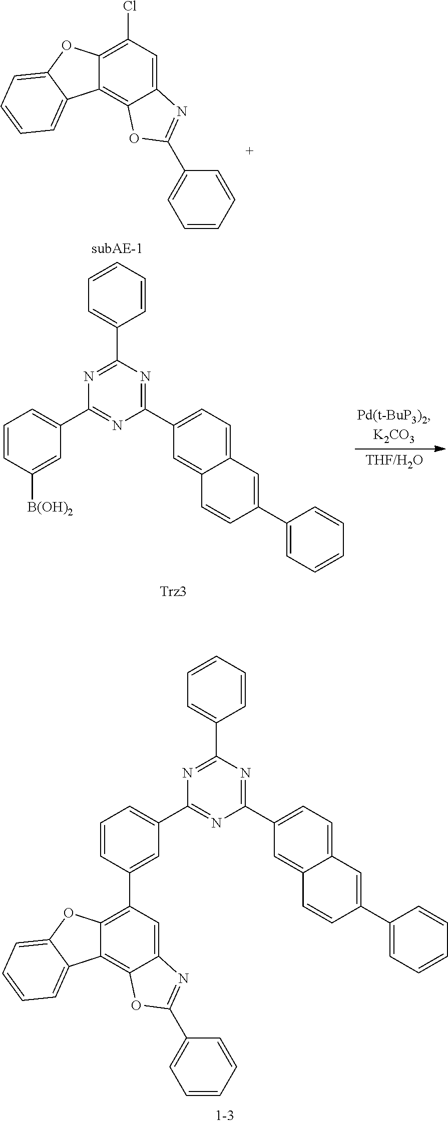 [SMILES: Clc1cc2nc(-c3ccccc3)oc2c2c1oc1ccccc12 ; OB(O)c1cccc(-c2nc(-c3ccccc3)nc(-c3ccc4cc(-c5ccccc5)ccc4c3)n2)c1 ; c1ccc(-c2ccc3cc(-c4nc(-c5ccccc5)nc(-c5cccc(-c6cc7nc(-c8ccccc8)oc7c7c6oc6ccccc67)c5)n4)ccc3c2)cc1]

Compound subAE-1 (15 g, 46.9 mmol) and Trz3 (22.5 g, 46.9 mmol) were added to 300 ml of THF under a nitrogen atmosphere, and the mixture was stirred and refluxed. Then, potassium carbonate (19.5 g, 140.7 mmol) was dissolved in 58 ml of water, and then added thereto. Thereafter, it was stirred sufficiently, followed by adding bis(tri-tert-butylphosphine)palladium(0) (0.2 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 25.3 g of Compound 1-3 (yield 75%, MS: [M+H]+=719).

### Preparation Example 1-4

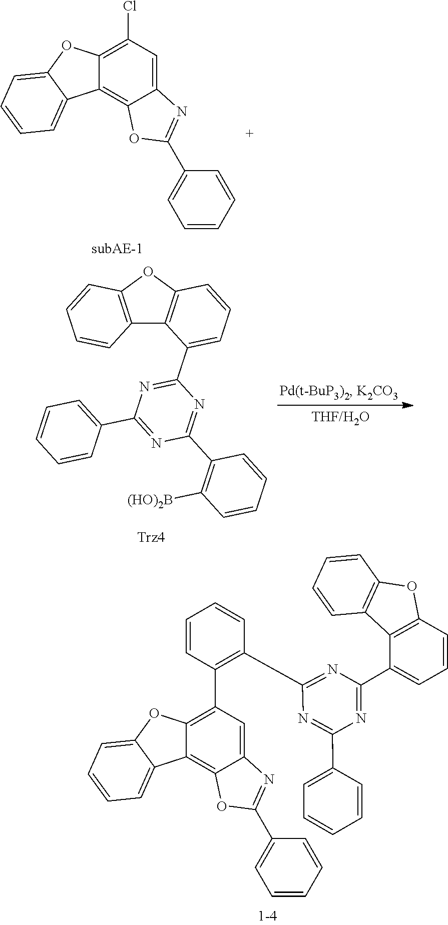 [SMILES: Clc1cc2nc(-c3ccccc3)oc2c2c1oc1ccccc12 ; OB(O)c1ccccc1-c1nc(-c2ccccc2)nc(-c2cccc3oc4ccccc4c23)n1 ; c1ccc(-c2nc(-c3ccccc3-c3cc4nc(-c5ccccc5)oc4c4c3oc3ccccc34)nc(-c3cccc4oc5ccccc5c34)n2)cc1]

Compound subAE-1 (15 g, 46.9 mmol) and Trz4 (20.8 g, 46.9 mmol) were added to 300 ml of THF under a nitrogen atmosphere, and the mixture was stirred and refluxed. Then, potassium carbonate (19.5 g, 140.7 mmol) was dissolved in 58 ml of water, and then added thereto. Thereafter, it was stirred sufficiently, followed by adding bis(tri-tert-butylphosphine)palladium(0) (0.2 g, 0.5 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 22.1 g of Compound 1-4 (yield 69%, MS: [M+H]+=683).

### Preparation Example 1-5

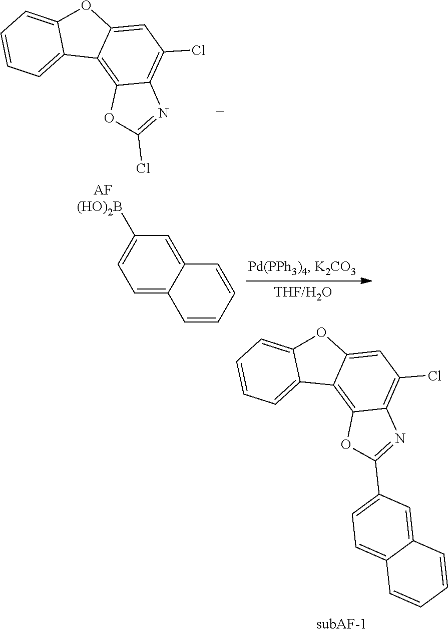

Compound AF (15 g, 53.9 mmol) and naphthalen-2-ylboronic acid (9.3 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)palladium(0) (0.6 g, 0.5 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.1 g of Compound subAF-1 (yield 66%, MS: [M+H]+=370).

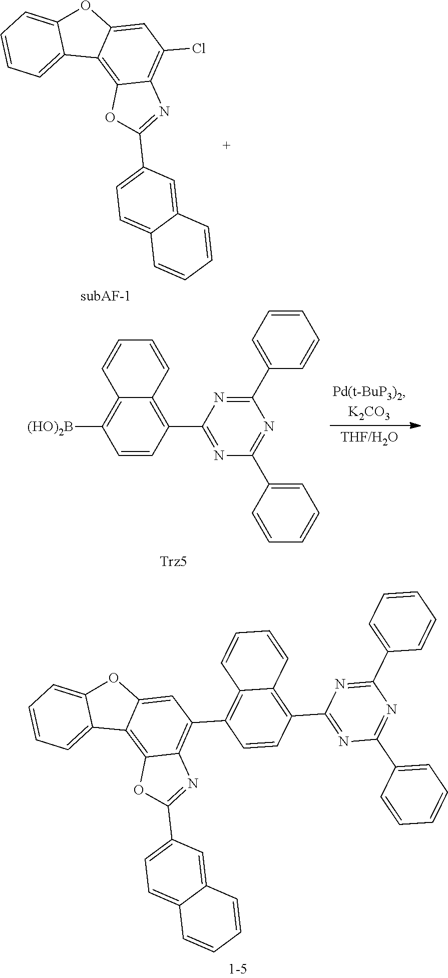 [SMILES: Clc1cc2oc3ccccc3c2c2oc(-c3ccc4ccccc4c3)nc12 ; OB(O)c1ccc(-c2nc(-c3ccccc3)nc(-c3ccccc3)n2)c2ccccc12 ; c1ccc(-c2nc(-c3ccccc3)nc(-c3ccc(-c4cc5oc6ccccc6c5c5oc(-c6ccc7ccccc7c6)nc45)c4ccccc34)n2)cc1]

Compound subAF-1 (15 g, 40.6 mmol) and Trz5 (16.4 g, 40.6 mmol) were added to 300 ml of THF under a nitrogen atmosphere, and the mixture was stirred and refluxed. Then, potassium carbonate (16.8 g, 121.7 mmol) was dissolved in 50 ml of water, and then added thereto. Thereafter, it was stirred sufficiently, followed by adding bis(tri-tert-butylphosphine)palladium(0) (0.2 g, 0.4 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 17.4 g of Compound 1-5 (yield 62%, MS: [M+H]+=693).

### Preparation Example 1-6

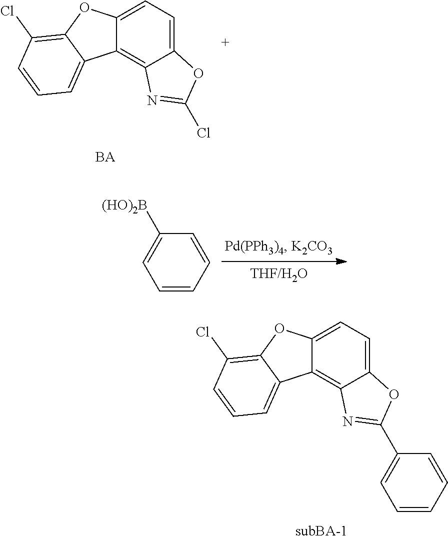

Compound BA (15 g, 53.9 mmol) and phenylboronic acid (6.6 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)-palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.6 g of Compound subBA-1 (yield 79%, MS: [M+H]+=320).

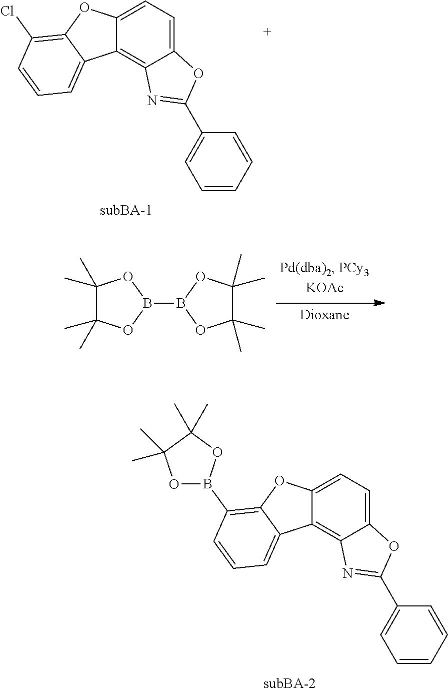

Compound subBA-1 (15 g, 46.9 mmol) and bis(pinacolato)diboron (13.1 g, 51.6 mmol) were added to 300 ml of 1,4-dioxane under a nitrogen atmosphere, and the mixture was stirred and refluxed. Then, potassium acetate (6.9 g, 70.4 mmol) was added and stirred sufficiently, followed by adding bis(dibenzylideneacetone)palladium(0) (0.8 g, 1.4 mmol) and tricyclohexylphosphine (0.8 g, 2.8 mmol). After 8 hours of reaction, cooling was performed to room temperature. Then, the organic layer was separated using chloroform and water, and distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.1 g of Compound subBA-2 (yield 68%, MS: [M+H]+=412).

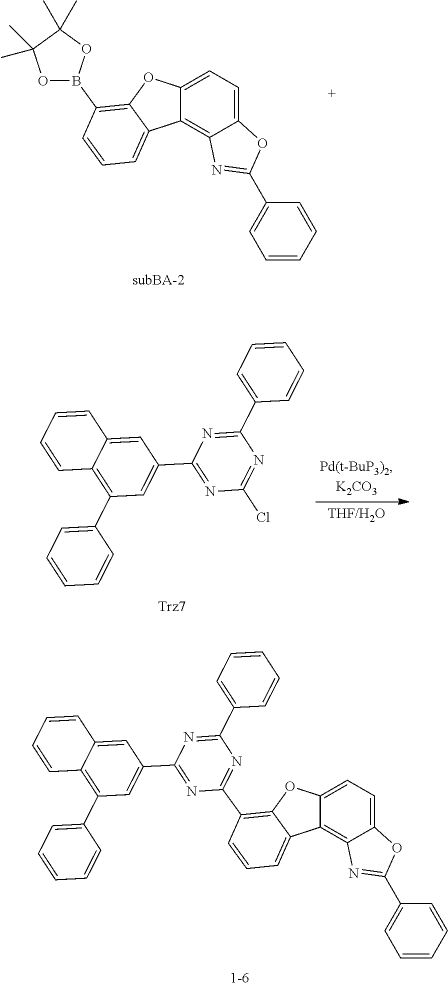 [SMILES: CC1(C)OB(c2cccc3c2oc2ccc4oc(-c5ccccc5)nc4c23)OC1(C)C ; Clc1nc(-c2ccccc2)nc(-c2cc(-c3ccccc3)c3ccccc3c2)n1 ; c1ccc(-c2nc(-c3cc(-c4ccccc4)c4ccccc4c3)nc(-c3cccc4c3oc3ccc5oc(-c6ccccc6)nc5c34)n2)cc1]

Compound subBA-2 (15 g, 36.5 mmol) and Trz7 (14.4 g, 36.5 mmol) were added to 300 ml of THF under a nitrogen atmosphere, and the mixture was stirred and refluxed. Then, potassium carbonate (15.1 g, 109.4 mmol) was dissolved in 45 ml of water, and then added thereto. Thereafter, it was stirred sufficiently, followed by adding bis(tri-tert-butylphosphine)palladium(0) (0.2 g, 0.4 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.5 g of Compound 1-6 (yield 66%, MS: [M+H]+=643).

### Preparation Example 1-7

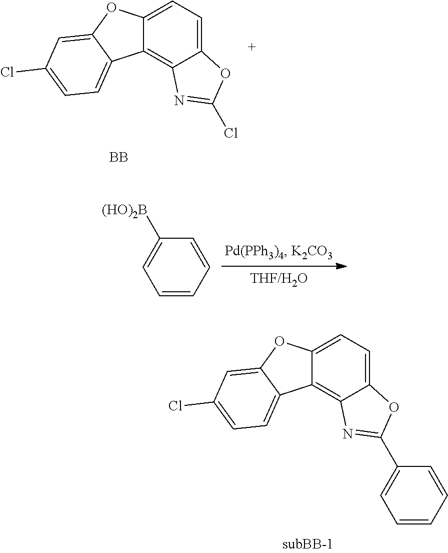

Compound BB (15 g, 53.9 mmol) and phenylboronic acid (6.6 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)palladium(0) (0.6 g, 0.5 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.2 g of Compound subBB-1 (yield 65%, MS: [M+H]+=320).

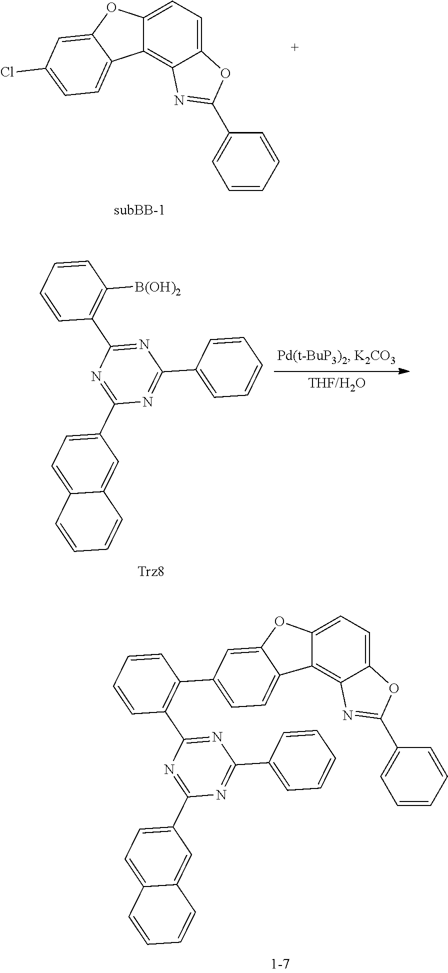 [SMILES: Clc1ccc2c(c1)oc1ccc3oc(-c4ccccc4)nc3c12 ; OB(O)c1ccccc1-c1nc(-c2ccccc2)nc(-c2ccc3ccccc3c2)n1 ; c1ccc(-c2nc(-c3ccc4ccccc4c3)nc(-c3ccccc3-c3ccc4c(c3)oc3ccc5oc(-c6ccccc6)nc5c34)n2)cc1]

Compound subBB-1 (15 g, 46.9 mmol) and Trz8 (18.9 g, 46.9 mmol) were added to 300 ml of THF under a nitrogen atmosphere, and the mixture was stirred and refluxed. Then, potassium carbonate (19.5 g, 140.7 mmol) was dissolved in 58 ml of water, and then added thereto. Thereafter, it was stirred sufficiently, followed by adding bis(tri-tert-butylphosphine)palladium(0) (0.2 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 18.1 g of Compound 1-7 (yield 60%, MS: [M+H]+=643).

### Preparation Example 1-8

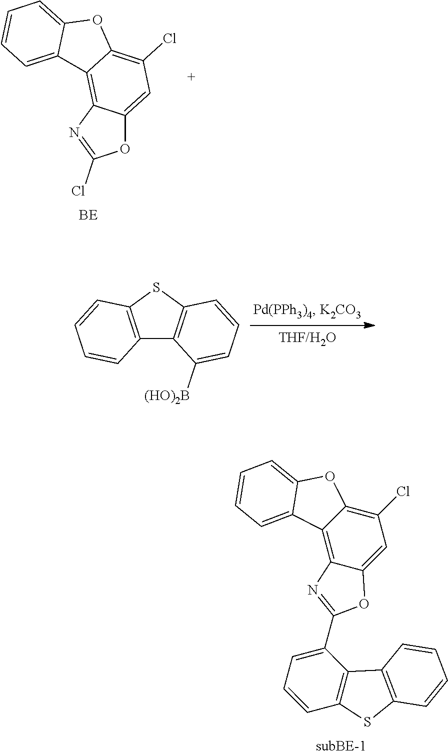

Compound BE (15 g, 53.9 mmol) and dibenzo[b,d]thiophen-1-ylboronic acid (12.3 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)palladium(0) (0.6 g, 0.5 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 16.7 g of Compound subBE-1 (yield 73%, MS: [M+H]+=426).

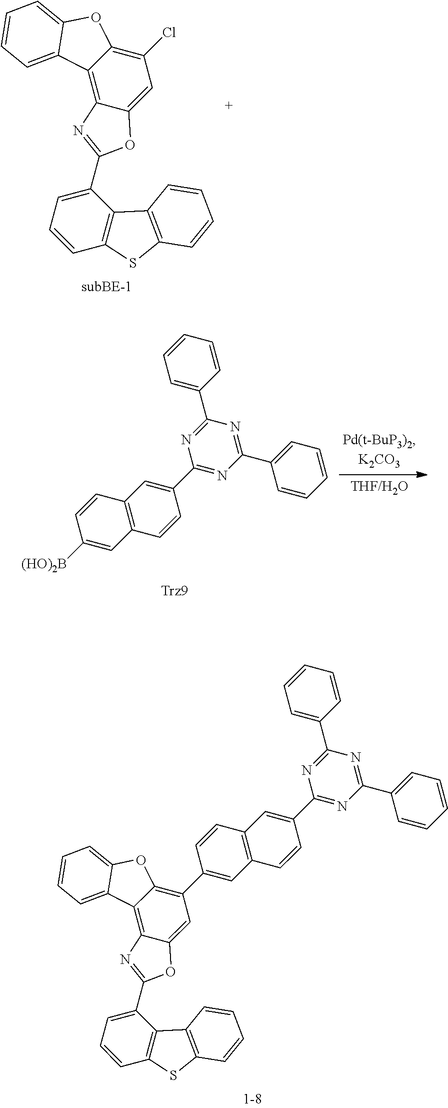 [SMILES: Clc1cc2oc(-c3cccc4sc5ccccc5c34)nc2c2c1oc1ccccc12 ; OB(O)c1ccc2cc(-c3nc(-c4ccccc4)nc(-c4ccccc4)n3)ccc2c1 ; c1ccc(-c2nc(-c3ccccc3)nc(-c3ccc4cc(-c5cc6oc(-c7cccc8sc9ccccc9c78)nc6c6c5oc5ccccc56)ccc4c3)n2)cc1]

Compound subBE-1 (15 g, 35.2 mmol) and Trz9 (14.2 g, 35.2 mmol) were added to 300 ml of THF under a nitrogen atmosphere, and the mixture was stirred and refluxed. Then, potassium carbonate (14.6 g, 105.7 mmol) was dissolved in 44 ml of water, and then added thereto. Thereafter, it was stirred sufficiently, followed by adding bis(tri-tert-butylphosphine)palladium(0) (0.2 g, 0.4 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 18.2 g of Compound 1-8 (yield 69%, MS: [M+H]+=749).

### Preparation Example 1-9

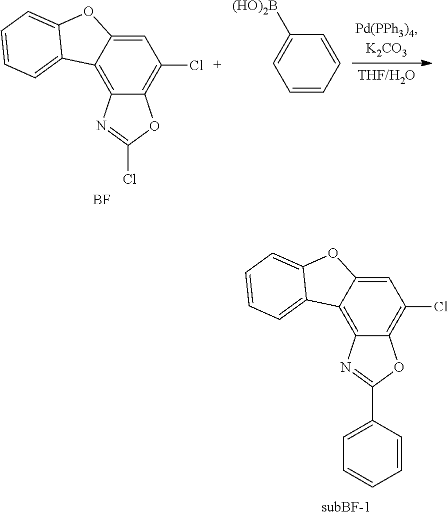

Compound BF (15 g, 53.9 mmol) and phenylboronic acid (6.6 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)-palladium(0) (0.6 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.5 g of Compound subBF-1 (yield 61%, MS: [M+H]+=320).

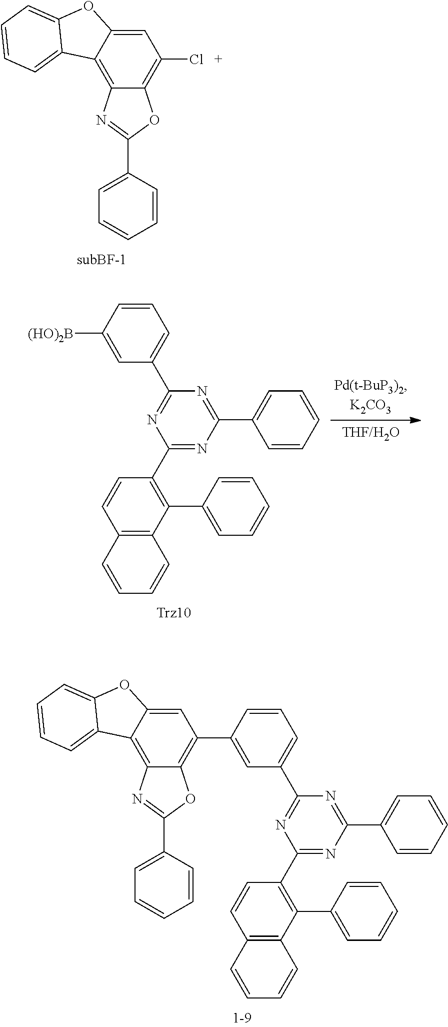

Compound subBF-1 (15 g, 46.9 mmol) and Trz10 (22.5 g, 46.9 mmol) were added to 300 ml of THF under a nitrogen atmosphere, and the mixture was stirred and refluxed. Then, potassium carbonate (19.5 g, 140.7 mmol) was dissolved in 58 ml of water, and then added thereto. Thereafter, it was stirred sufficiently, followed by adding bis(tri-tert-butylphosphine)palladium(0) (0.2 g, 0.5 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.2 g of Compound 1-9 (yield 60%, MS: [M+H]+=719).

### Preparation Example 1-10

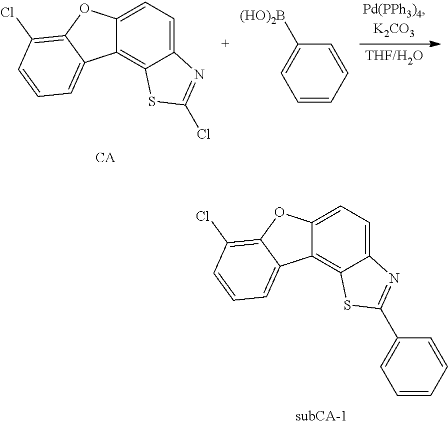

Compound CA (15 g, 51 mmol) and phenylboronic acid (6.2 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.4 g of Compound subCA-1 (yield 61%, MS: [M+H]+=336).

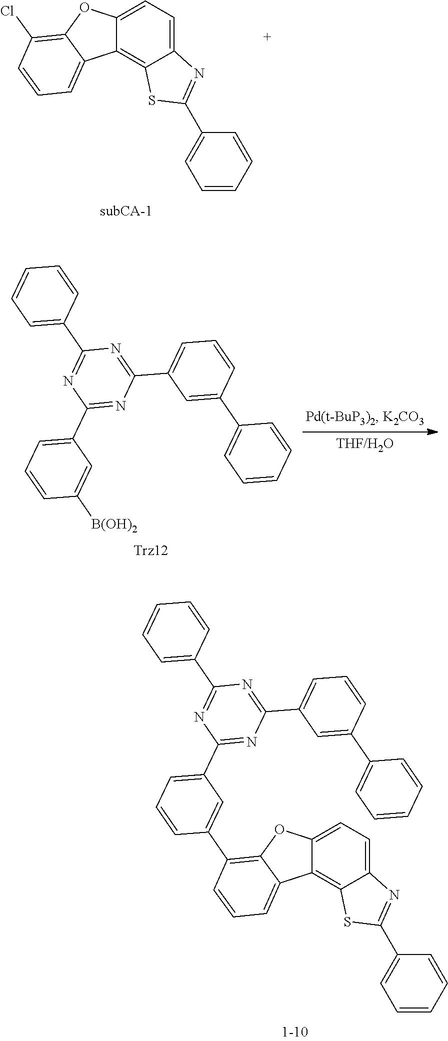 [SMILES: Clc1cccc2c1oc1ccc3nc(-c4ccccc4)sc3c12 ; OB(O)c1cccc(-c2nc(-c3ccccc3)nc(-c3cccc(-c4ccccc4)c3)n2)c1 ; c1ccc(-c2cccc(-c3nc(-c4ccccc4)nc(-c4cccc(-c5cccc6c5oc5ccc7nc(-c8ccccc8)sc7c56)c4)n3)c2)cc1]

Compound subCA-1 (15 g, 44.7 mmol) and Trz12 (19.2 g, 44.7 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.5 g of Compound 1-10 (yield 67%, MS: [M+H]+=685).

### Preparation Example 1-11

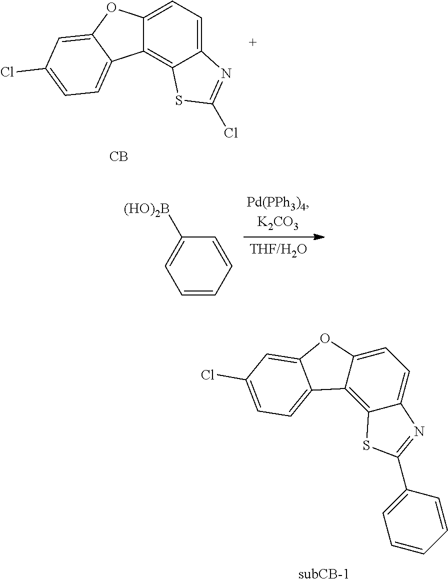

Compound CB (15 g, 51 mmol) and phenylboronic acid (6.2 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.2 g of Compound subCB-1 (yield 77%, MS: [M+H]+=336).

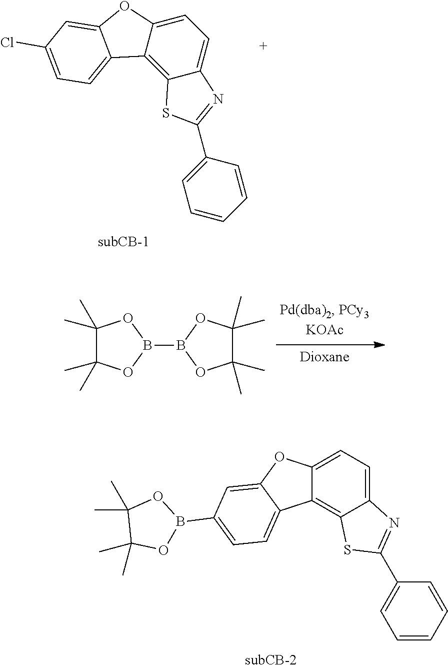

Compound subCB-1 (15 g, 44.7 mmol) and bis(pinacolato)diboron (12.5 g, 49.1 mmol) were added to 300 ml of 1,4-dioxane under a nitrogen atmosphere, and the mixture was stirred and refluxed. Then, potassium acetate (6.6 g, 67 mmol) was added and stirred sufficiently, followed by adding bis(dibenzylideneacetone)palladium(0) (0.8 g, 1.3 mmol) and tricyclohexylphosphine (0.8 g, 2.7 mmol). After 9 hours of reaction, cooling was performed to room temperature. Then, the organic layer was separated using chloroform and water, and distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.9 g of Compound subCB-2 (yield 73%, MS: [M+H]+=428).

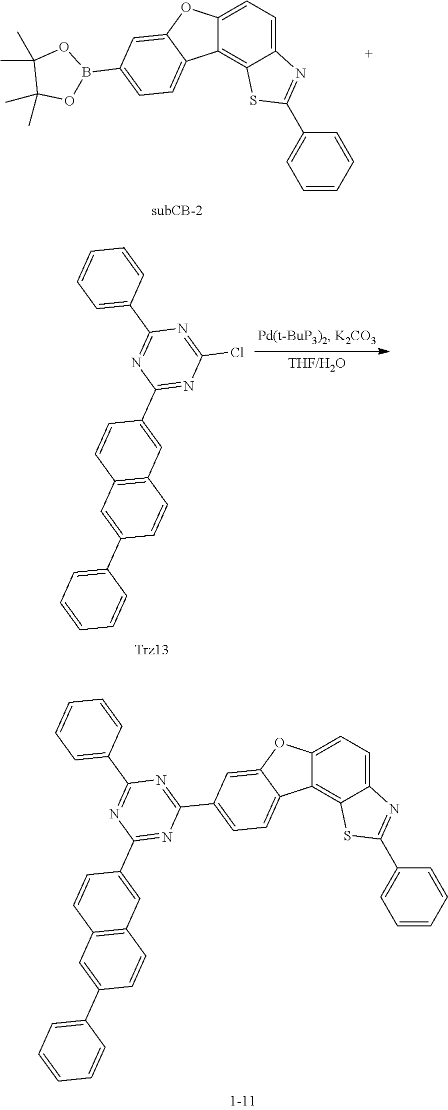 [SMILES: CC1(C)OB(c2ccc3c(c2)oc2ccc4nc(-c5ccccc5)sc4c23)OC1(C)C ; Clc1nc(-c2ccccc2)nc(-c2ccc3cc(-c4ccccc4)ccc3c2)n1 ; c1ccc(-c2ccc3cc(-c4nc(-c5ccccc5)nc(-c5ccc6c(c5)oc5ccc7nc(-c8ccccc8)sc7c56)n4)ccc3c2)cc1]

Compound subCB-2 (15 g, 35.1 mmol) and Trz13 (13.8 g, 35.1 mmol) were added to 300 ml of THF under a nitrogen atmosphere, and the mixture was stirred and refluxed. Then, potassium carbonate (14.6 g, 105.3 mmol) was dissolved in 44 ml of water, and then added thereto. Thereafter, it was stirred sufficiently, followed by adding bis(tri-tert-butylphosphine)palladium(0) (0.2 g, 0.4 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 16.6 g of Compound 1-11 (yield 72%, MS: [M+H]+=659).

### Preparation Example 1-12

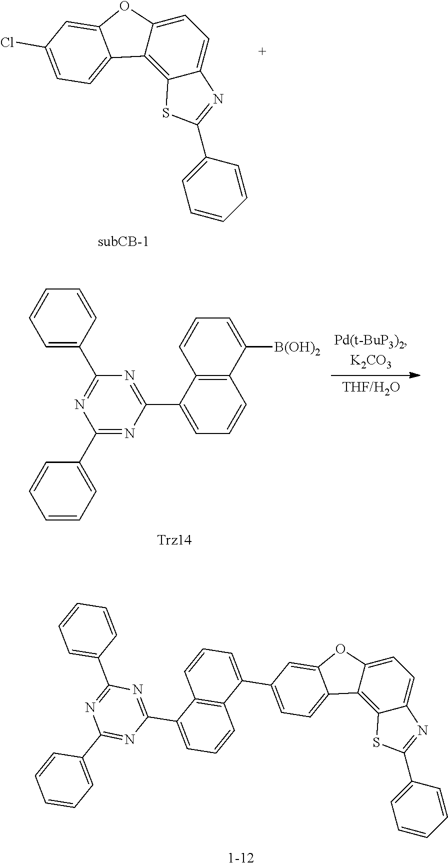 [SMILES: Clc1ccc2c(c1)oc1ccc3nc(-c4ccccc4)sc3c12 ; OB(O)c1cccc2c(-c3nc(-c4ccccc4)nc(-c4ccccc4)n3)cccc12 ; c1ccc(-c2nc(-c3ccccc3)nc(-c3cccc4c(-c5ccc6c(c5)oc5ccc7nc(-c8ccccc8)sc7c56)cccc34)n2)cc1]

Compound subCB-1 (15 g, 36.5 mmol) and Trz14 (14.7 g, 36.5 mmol) were added to 300 ml of THF under a nitrogen atmosphere, and the mixture was stirred and refluxed. Then, potassium carbonate (15.1 g, 109.4 mmol) was dissolved in 45 ml of water, and then added thereto. Thereafter, it was stirred sufficiently, followed by adding bis(tri-tert-butylphosphine)palladium(0) (0.2 g, 0.4 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 17 g of Compound 1-12 (yield 71%, MS: [M+H]+=659).

### Preparation Example 1-13

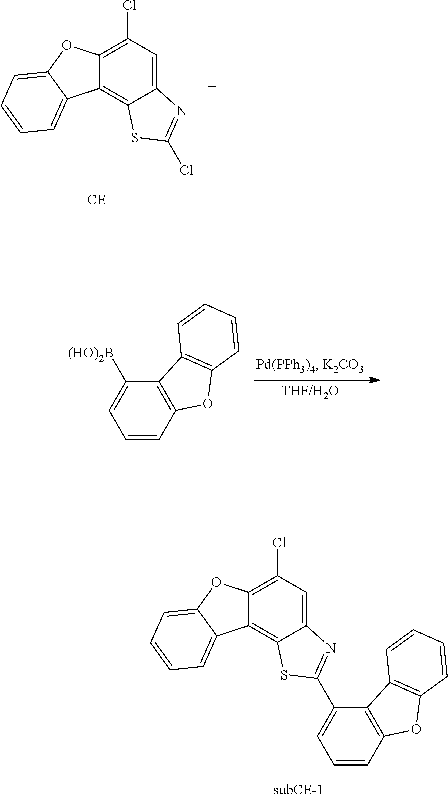 [SMILES: Clc1nc2cc(Cl)c3oc4ccccc4c3c2s1 ; OB(O)c1cccc2oc3ccccc3c12 ; Clc1cc2nc(-c3cccc4oc5ccccc5c34)sc2c2c1oc1ccccc12]

Compound CE (15 g, 51 mmol) and dibenzo[b,d]furan-1-ylboronic acid (10.8 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.7 g of Compound subCE-1 (yield 63%, MS: [M+H]+=426).

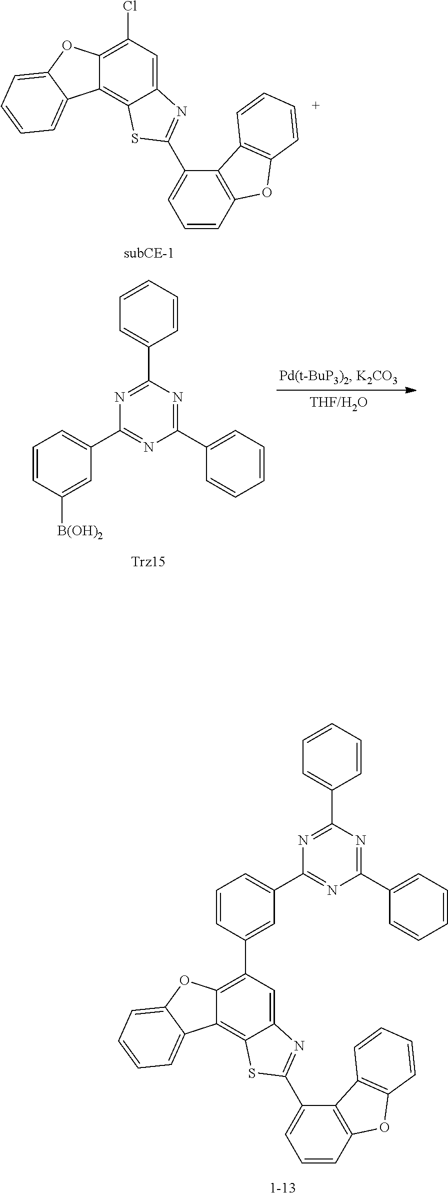 [SMILES: Clc1cc2nc(-c3cccc4oc5ccccc5c34)sc2c2c1oc1ccccc12 ; OB(O)c1cccc(-c2nc(-c3ccccc3)nc(-c3ccccc3)n2)c1 ; c1ccc(-c2nc(-c3ccccc3)nc(-c3cccc(-c4cc5nc(-c6cccc7oc8ccccc8c67)sc5c5c4oc4ccccc45)c3)n2)cc1]

Compound subCE-1 (15 g, 35.2 mmol) and Trz15 (12.4 g, 35.2 mmol) were added to 300 ml of THF under a nitrogen atmosphere, and the mixture was stirred and refluxed. Then, potassium carbonate (14.6 g, 105.7 mmol) was dissolved in 44 ml of water, and then added thereto. Thereafter, it was stirred sufficiently, followed by adding bis(tri-tert-butylphosphine)palladium(0) (0.2 g, 0.4 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.2 g of Compound 1-13 (yield 62%, MS: [M+H]+=699).

### Preparation Example 1-14

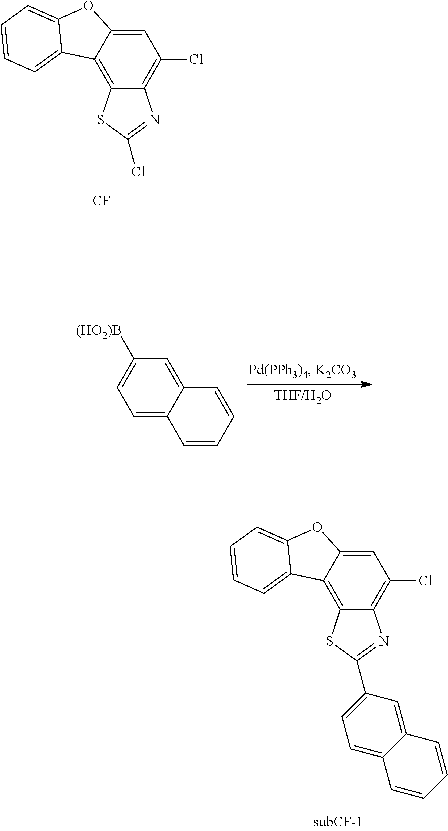

Compound CF (15 g, 51 mmol) and naphthalen-2-ylboronic acid (8.8 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)-palladium(0) (0.6 g, 0.5 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.9 g of Compound subCF-1 (yield 76%, MS: [M+H]+=386).

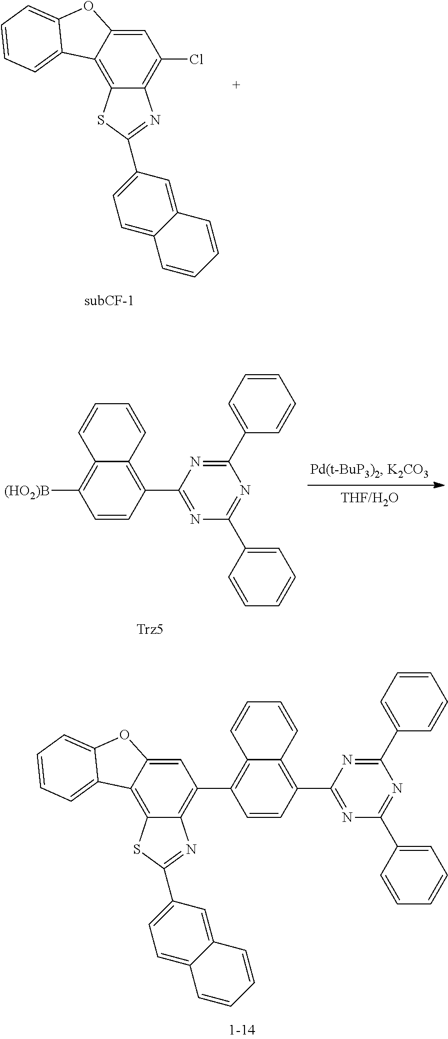

Compound subCF-1 (15 g, 38.9 mmol) and Trz5 (15.7 g, 38.9 mmol) were added to 300 ml of THF under a nitrogen atmosphere, and the mixture was stirred and refluxed. Then, potassium carbonate (16.1 g, 116.6 mmol) was dissolved in 48 ml of water, and then added thereto. Thereafter, it was stirred sufficiently, followed by adding bis(tri-tert-butylphosphine)palladium(0) (0.2 g, 0.4 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 18.2 g of Compound 1-14 (yield 66%, MS: [M+H]+=709).

### Preparation Example 1-15

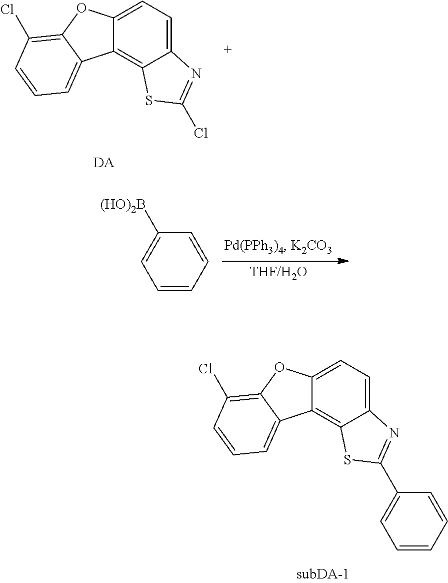 [SMILES: Clc1nc2ccc3oc4c(Cl)cccc4c3c2s1 ; "DA" ; OB(O)c1ccccc1 ; Clc1cccc2c1oc1ccc3nc(-c4ccccc4)sc3c12]

Compound DA (15 g, 51 mmol) and phenylboronic acid (6.2 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.6 g of Compound subDA-1 (yield 68%, MS: [M+H]+=336).

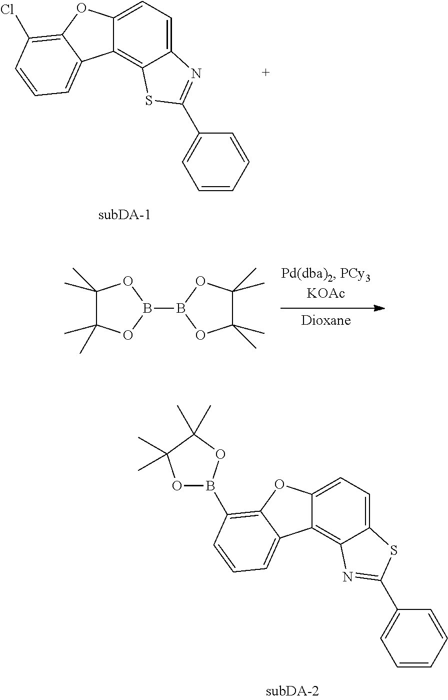

Compound subDA-1 (15 g, 44.7 mmol) and bis(pinacolato)diboron (12.5 g, 49.1 mmol) were added to 300 ml of 1,4-dioxane under a nitrogen atmosphere, and the mixture was stirred and refluxed. Then, potassium acetate (6.6 g, 67 mmol) was added and stirred sufficiently, followed by adding bis(dibenzylideneacetone)palladium(0) (0.8 g, 1.3 mmol) and tricyclohexylphosphine (0.8 g, 2.7 mmol). After 7 hours of reaction, cooling was performed to room temperature. Then, the organic layer was separated using chloroform and water, and distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.4 g of Compound subDA-2 (yield 70%, MS: [M+H]+=428).

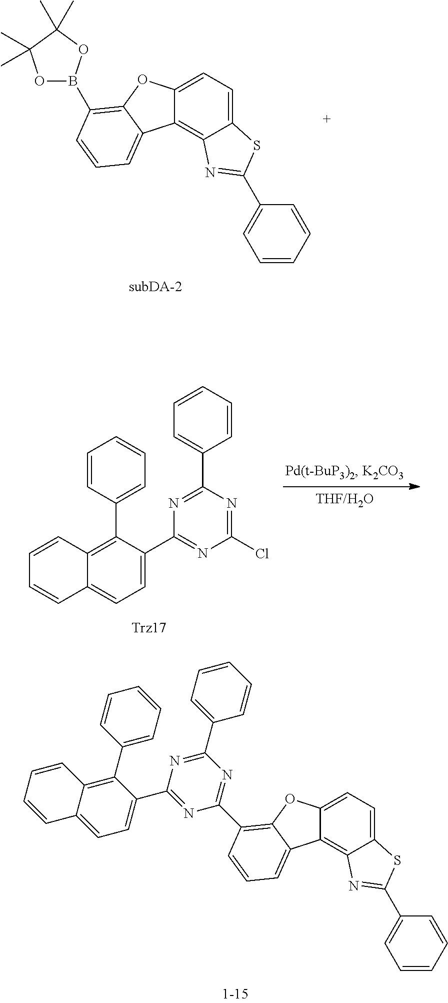 [SMILES: CC1(C)OB(c2cccc3c2oc2ccc4sc(-c5ccccc5)nc4c23)OC1(C)C ; Clc1nc(-c2ccccc2)nc(-c2ccc3ccccc3c2-c2ccccc2)n1 ; c1ccc(-c2nc(-c3ccc4ccccc4c3-c3ccccc3)nc(-c3cccc4c3oc3ccc5sc(-c6ccccc6)nc5c34)n2)cc1]

Compound subDA-2 (15 g, 35.1 mmol) and Trz17 (13.8 g, 35.1 mmol) were added to 300 ml of THF under a nitrogen atmosphere, and the mixture was stirred and refluxed. Then, potassium carbonate (14.6 g, 105.3 mmol) was dissolved in 44 ml of water, and then added thereto. Thereafter, it was stirred sufficiently, followed by adding bis(tri-tert-butylphosphine)palladium(0) (0.2 g, 0.4 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.6 g of Compound 1-15 (yield 63%, MS: [M+H]+=659).

### Preparation Example 1-16

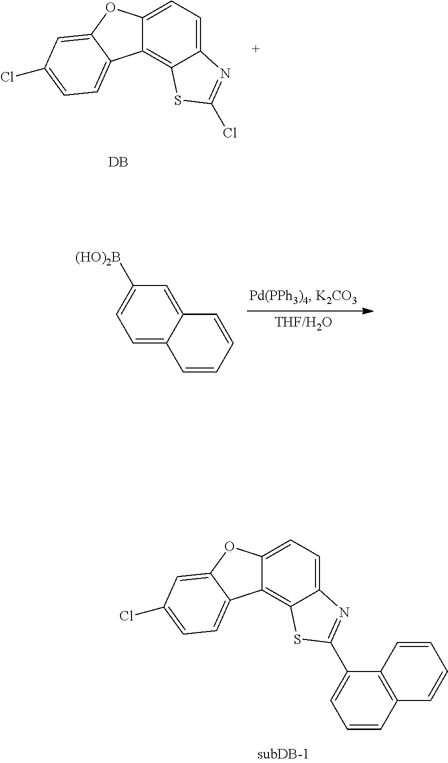 [SMILES: Clc1ccc2c(c1)oc1ccc3nc(Cl)sc3c12 ; "DB" ; OB(O)c1ccc2ccccc2c1 ; Clc1ccc2c(c1)oc1ccc3nc(-c4cccc5ccccc45)sc3c12]

Compound DB (15 g, 51 mmol) and naphthalen-2-ylboronic acid (8.8 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)-palladium(0) (0.6 g, 0.5 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.4 g of Compound subDB-1 (yield 68%, MS: [M+H]+=386).

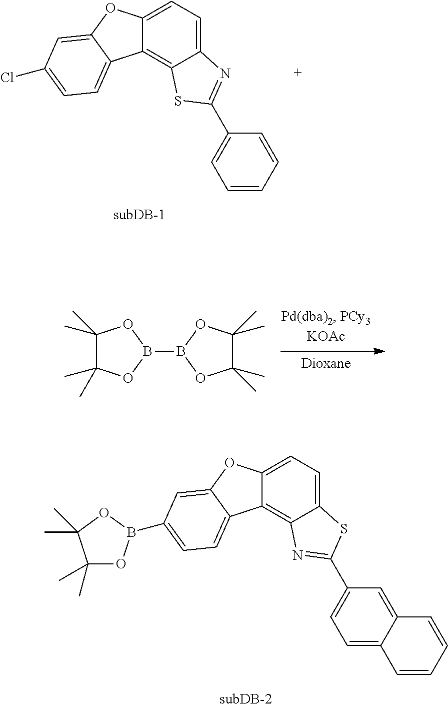

Compound subDB-1 (15 g, 39 mmol) and bis(pinacolato)diboron (10.9 g, 42.9 mmol) were added to 300 ml of 1,4-dioxane under a nitrogen atmosphere, and the mixture was stirred and refluxed. Then, potassium acetate (5.7 g, 58.5 mmol) was added and stirred sufficiently, followed by adding bis(dibenzylideneacetone)palladium(0) (0.7 g, 1.2 mmol) and tricyclohexylphosphine (0.7 g, 2.3 mmol). After 7 hours of reaction, cooling was performed to room temperature. Then, the organic layer was separated using chloroform and water, and distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.9 g of Compound subDB-2 (yield 75%, MS: [M+H]+=478).

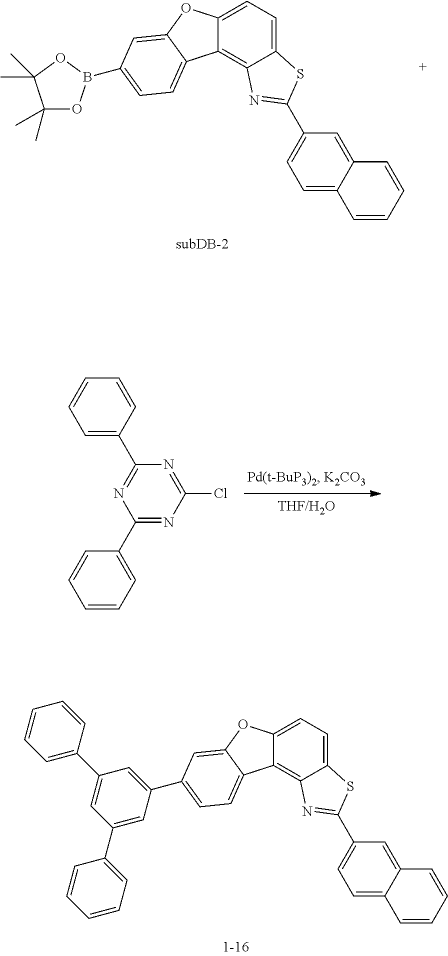 [SMILES: CC1(C)OB(c2ccc3c(c2)oc2ccc4sc(-c5ccc6ccccc6c5)nc4c23)OC1(C)C ; Clc1nc(-c2ccccc2)nc(-c2ccccc2)n1 ; c1ccc(-c2cc(-c3ccccc3)cc(-c3ccc4c(c3)oc3ccc5sc(-c6ccc7ccccc7c6)nc5c34)c2)cc1]

Compound subDB-2 (15 g, 31.4 mmol) and Trz2 (8.4 g, 31.4 mmol) were added to 300 ml of THF under a nitrogen atmosphere, and the mixture was stirred and refluxed. Then, potassium carbonate (13 g, 94.3 mmol) was dissolved in 39 ml of water, and then added thereto. Thereafter, it was stirred sufficiently, followed by adding bis(tri-tert-butylphosphine)palladium(0) (0.2 g, 0.3 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13 g of Compound 1-16 (yield 71%, MS: [M+H]+=583).

### Preparation Example 1-17

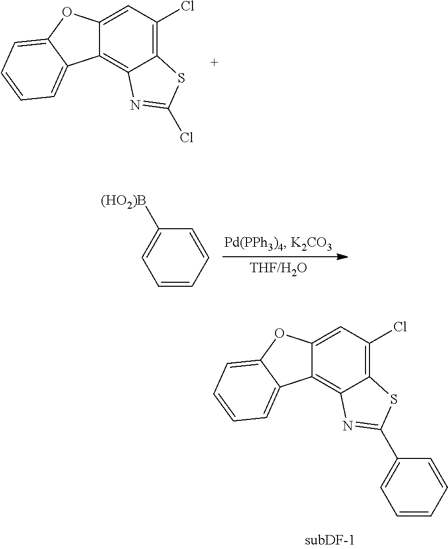

Compound DF (15 g, 51 mmol) and phenylboronic acid (6.2 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.1 g of Compound subDF-1 (yield 65%, MS: [M+H]+=336).

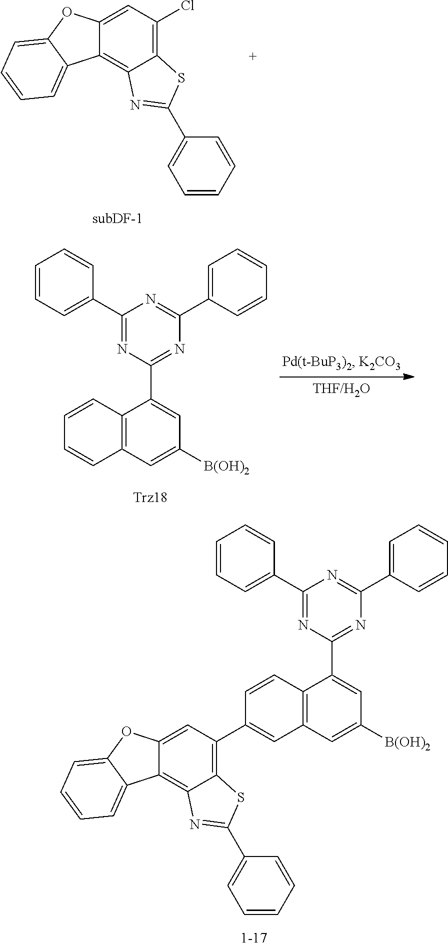 [SMILES: Clc1cc2oc3ccccc3c2c2nc(-c3ccccc3)sc12 ; OB(O)c1cc(-c2nc(-c3ccccc3)nc(-c3ccccc3)n2)c2ccccc2c1 ; OB(O)c1cc(-c2nc(-c3ccccc3)nc(-c3ccccc3)n2)c2ccc(-c3cc4oc5ccccc5c4c4nc(-c5ccccc5)sc34)cc2c1]

Compound subDF-1 (15 g, 44.7 mmol) and Trz18 (18 g, 44.7 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 18.2 g of Compound 1-17 (yield 62%, MS: [M+H]+=659).

### Preparation Example 2-1

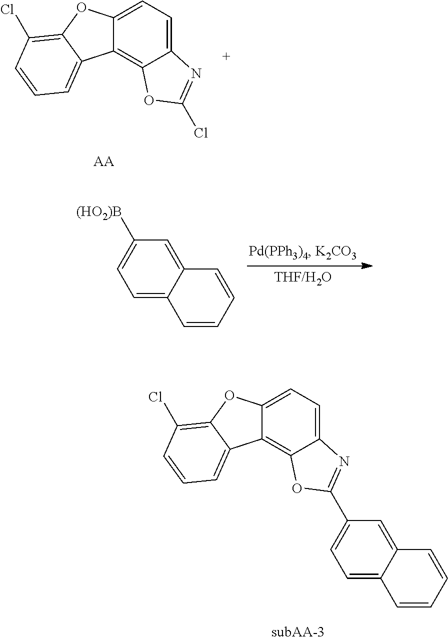

Compound AA (15 g, 53.9 mmol) and naphthalen-2-ylboronic acid (9.3 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)palladium(0) (0.6 g, 0.5 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.3 g of Compound subAA-3 (yield 77%, MS: [M+H]+=370).

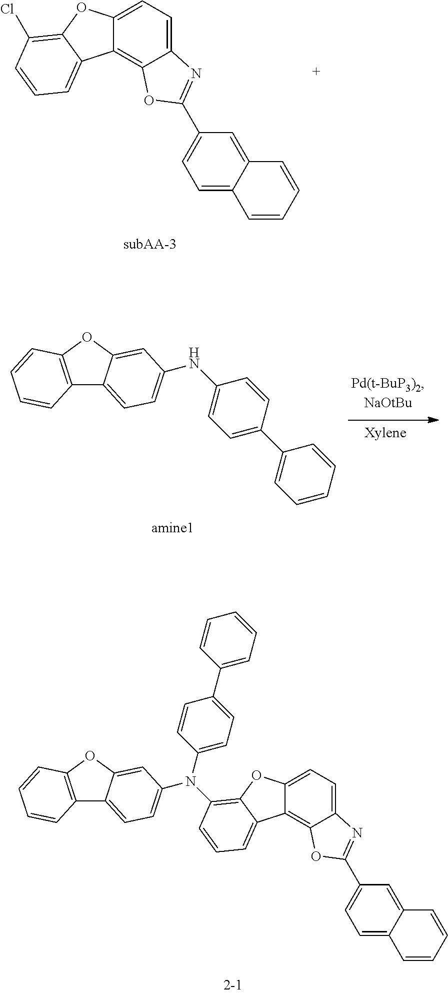 [SMILES: Clc1cccc2c1oc1ccc3nc(-c4ccc5ccccc5c4)oc3c12 ; c1ccc(-c2ccc(Nc3ccc4c(c3)oc3ccccc34)cc2)cc1 ; c1ccc(-c2ccc(N(c3ccc4c(c3)oc3ccccc34)c3cccc4c3oc3ccc5nc(-c6ccc7ccccc7c6)oc5c34)cc2)cc1]

Compound subAA-3 (10 g, 27 mmol), Compound amine1 (9.1 g, 27 mmol), and sodium tert-butoxide (8.6 g, 40.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 2 hours, the reaction was completed, and the solvent was removed under reduced pressure after cooling to room temperature. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10.8 g of Compound 2-1 (yield 60%, MS: [M+H]+=669).

### Preparation Example 2-2

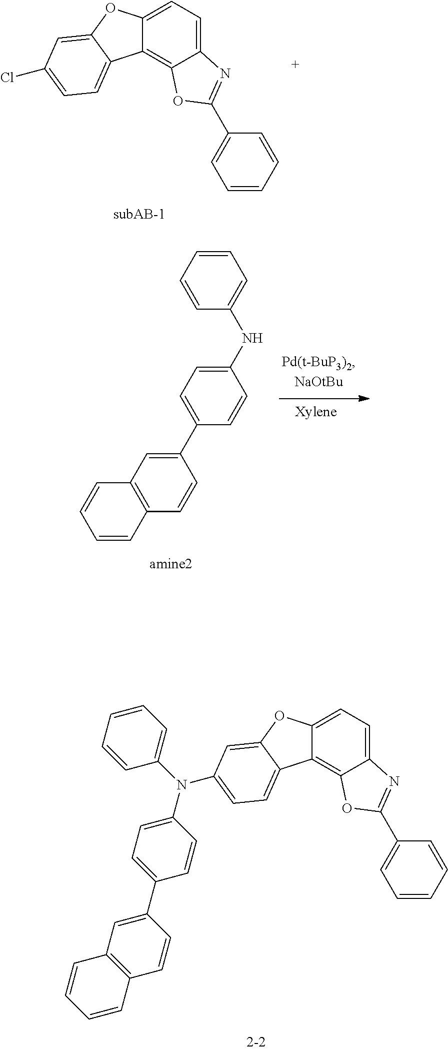 [SMILES: Clc1ccc2c(c1)oc1ccc3nc(-c4ccccc4)oc3c12 ; c1ccc(Nc2ccc(-c3ccc4ccccc4c3)cc2)cc1 ; c1ccc(-c2nc3ccc4oc5cc(N(c6ccccc6)c6ccc(-c7ccc8ccccc8c7)cc6)ccc5c4c3o2)cc1]

Compound subAB-1 (10 g, 31.3 mmol), Compound amine2 (9.2 g, 31.3 mmol), and sodium tert-butoxide (10 g, 46.9 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 2 hours, the reaction was completed, and the solvent was removed under reduced pressure after cooling to room temperature. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.9 g of Compound 2-2 (yield 66%, MS: [M+H]+=579).

### Preparation Example 2-3

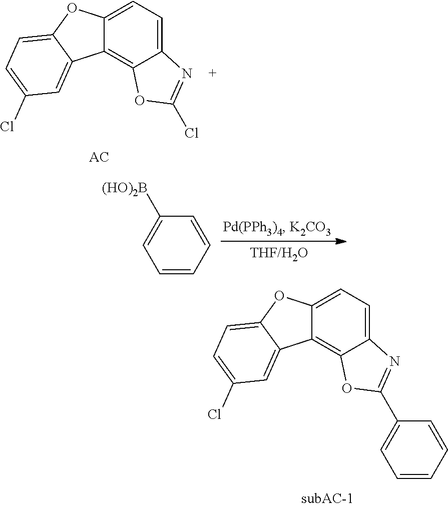 [SMILES: Clc1ccc2oc3ccc4nc(Cl)oc4c3c2c1 ; OB(O)c1ccccc1 ; Clc1ccc2oc3ccc4nc(-c5ccccc5)oc4c3c2c1]

Compound AC (15 g, 53.9 mmol) and phenylboronic acid (6.6 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)-palladium(0) (0.6 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.1 g of Compound subAC-1 (yield 76%, MS: [M+H]+=320).

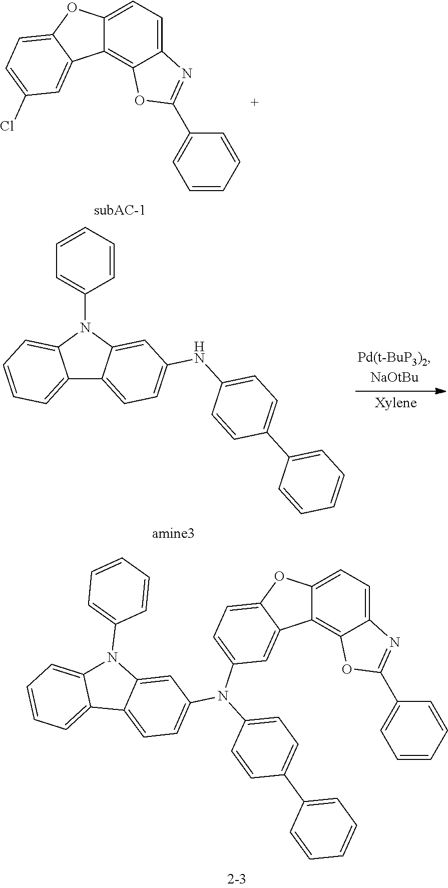 [SMILES: Clc1ccc2oc3ccc4nc(-c5ccccc5)oc4c3c2c1 ; c1ccc(-c2ccc(Nc3ccc4c5ccccc5n(-c5ccccc5)c4c3)cc2)cc1 ; c1ccc(-c2ccc(N(c3ccc4oc5ccc6nc(-c7ccccc7)oc6c5c4c3)c3ccc4c5ccccc5n(-c5ccccc5)c4c3)cc2)cc1]

Compound subAC-1 (10 g, 31.3 mmol), Compound amine3 (12.8 g, 31.3 mmol), and sodium tert-butoxide (10 g, 46.9 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 3 hours, the reaction was completed, and the solvent was removed under reduced pressure after cooling to room temperature. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 15 g of Compound 2-3 (yield 69%, MS: [M+H]+=694).

### Preparation Example 2-4

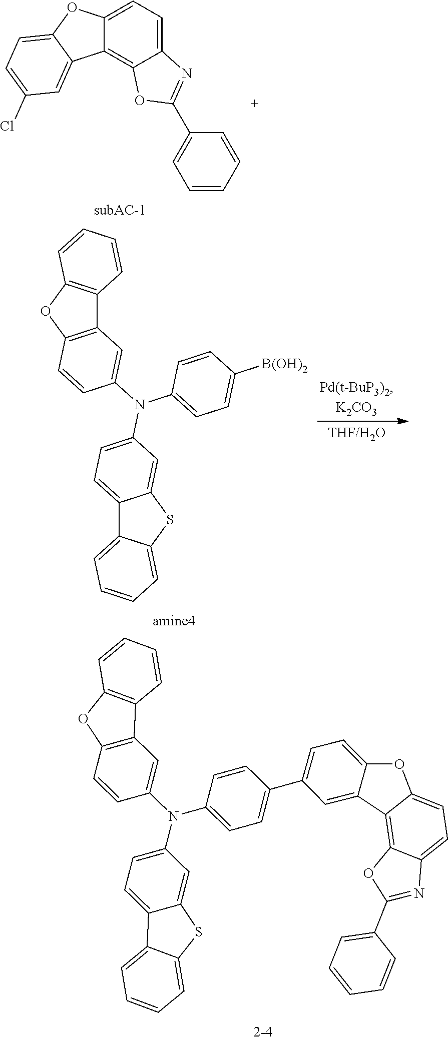

Compound subAC-1 (15 g, 46.9 mmol) and Compound amine4 (22.8 g, 46.9 mmol) were added to 300 ml of THF under a nitrogen atmosphere, and the mixture was stirred and refluxed. Then, potassium carbonate (19.5 g, 140.7 mmol) was dissolved in 58 ml of water, and then added thereto. Thereafter, it was stirred sufficiently, followed by adding bis(tri-tert-butylphosphine)palladium(0) (0.2 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.1 g of Compound 2-4 (yield 68%, MS: [M+H]+=725).

### Preparation Example 2-5

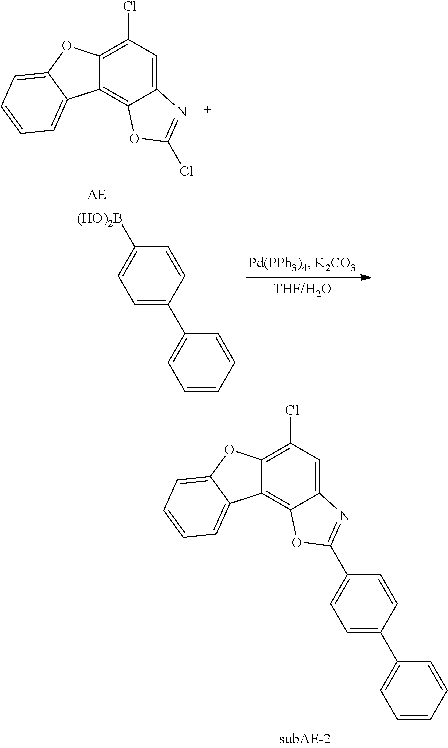 [SMILES: Clc1nc2cc(Cl)c3oc4ccccc4c3c2o1 ; OB(O)c1ccc(-c2ccccc2)cc1 ; Clc1cc2nc(-c3ccc(-c4ccccc4)cc3)oc2c2c1oc1ccccc12]

Compound AE (15 g, 53.9 mmol) and [1,1′-biphenyl]-4-ylboronic acid (10.7 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)palladium(0) (0.6 g, 0.5 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 17 g of Compound subAE-2 (yield 80%, MS: [M+H]+=396).

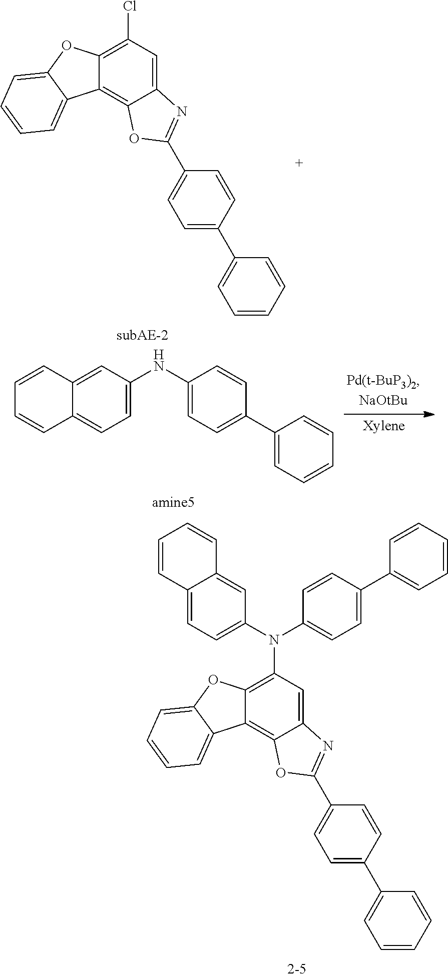 [SMILES: Clc1cc2nc(-c3ccc(-c4ccccc4)cc3)oc2c2c1oc1ccccc12 ; c1ccc(-c2ccc(Nc3ccc4ccccc4c3)cc2)cc1 ; c1ccc(-c2ccc(-c3nc4cc(N(c5ccc(-c6ccccc6)cc5)c5ccc6ccccc6c5)c5oc6ccccc6c5c4o3)cc2)cc1]

Compound subAE-2 (10 g, 25.3 mmol), Compound amine5 (7.5 g, 25.3 mmol), and sodium tert-butoxide (8 g, 37.9 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 2 hours, the reaction was completed, and the solvent was removed under reduced pressure after cooling to room temperature. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 8.3 g of Compound 2-5 (yield 50%, MS: [M+H]+=655).

### Preparation Example 2-6

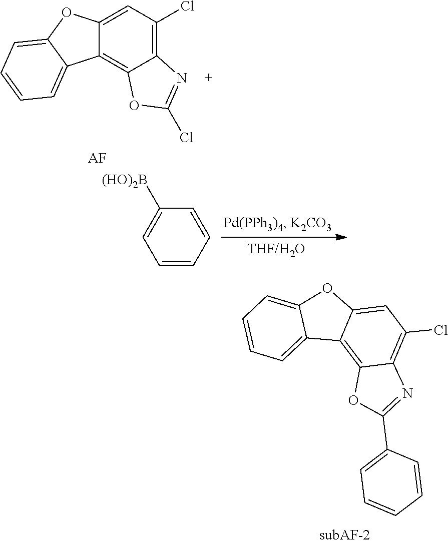 [SMILES: Clc1nc2c(Cl)cc3oc4ccccc4c3c2o1 ; "AF" ; OB(O)c1ccccc1 ; Clc1cc2oc3ccccc3c2c2oc(-c3ccccc3)nc12]

Compound AF (15 g, 53.9 mmol) and phenylboronic acid (6.6 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)-palladium(0) (0.6 g, 0.5 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.7 g of Compound subAF-2 (yield 74%, MS: [M+H]+=320).

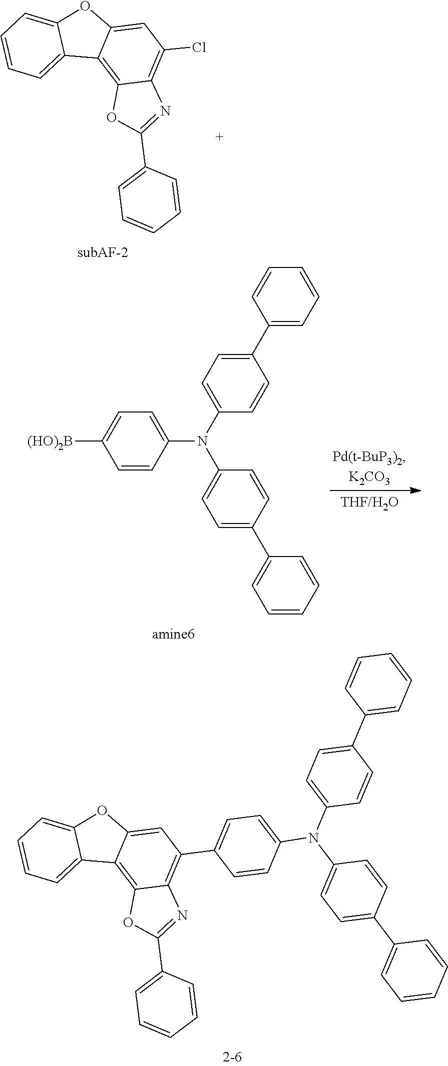 [SMILES: Clc1cc2oc3ccccc3c2c2oc(-c3ccccc3)nc12 ; OB(O)c1ccc(N(c2ccc(-c3ccccc3)cc2)c2ccc(-c3ccccc3)cc2)cc1 ; c1ccc(-c2ccc(N(c3ccc(-c4ccccc4)cc3)c3ccc(-c4cc5oc6ccccc6c5c5oc(-c6ccccc6)nc45)cc3)cc2)cc1]

Compound subAF-2 (15 g, 46.9 mmol) and Compound amine6 (20.7 g, 46.9 mmol) were added to 300 ml of THF under a nitrogen atmosphere, and the mixture was stirred and refluxed. Then, potassium carbonate (19.5 g, 140.7 mmol) was dissolved in 58 ml of water, and then added thereto. Thereafter, it was stirred sufficiently, followed by adding bis(tri-tert-butylphosphine)palladium(0) (0.2 g, 0.5 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.1 g of Compound 2-6 (yield 63%, MS: [M+H]+=681).

### Preparation Example 2-7

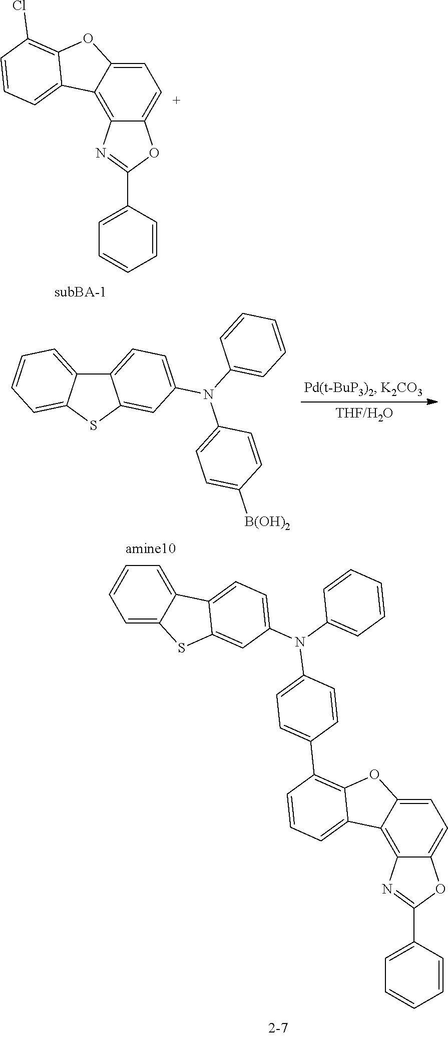 [SMILES: Clc1cccc2c1oc1ccc3oc(-c4ccccc4)nc3c12 ; OB(O)c1ccc(N(c2ccccc2)c2ccc3c(c2)sc2ccccc23)cc1 ; c1ccc(-c2nc3c(ccc4oc5c(-c6ccc(N(c7ccccc7)c7ccc8c(c7)sc7ccccc78)cc6)cccc5c43)o2)cc1]

Compound subBA-1 (15 g, 46.9 mmol) and Compound amine10 (18.5 g, 46.9 mmol) were added to 300 ml of THF under a nitrogen atmosphere, and the mixture was stirred and refluxed. Then, potassium carbonate (19.5 g, 140.7 mmol) was dissolved in 58 ml of water, and then added thereto. Thereafter, it was stirred sufficiently, followed by adding bis(tri-tert-butylphosphine)palladium(0) (0.2 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.2 g of Compound 2-7 (yield 78%, MS: [M+H]+=635).

### Preparation Example 2-8

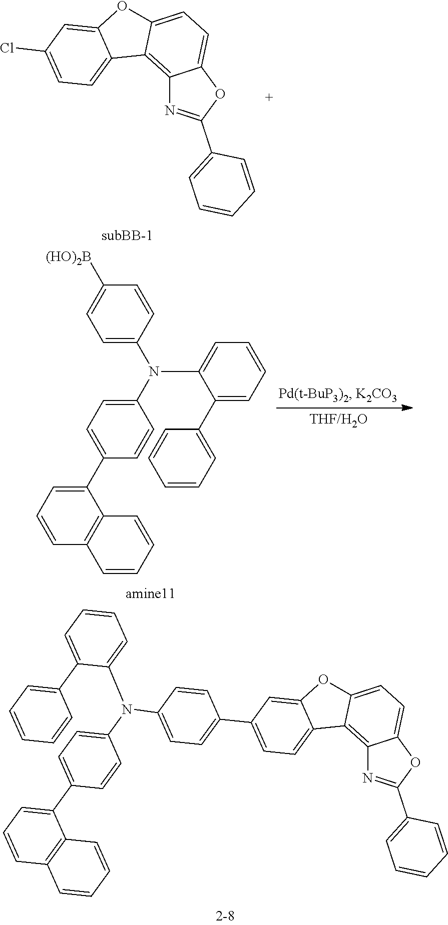

Compound subBB-1 (15 g, 46.9 mmol) and Compound amine11 (23.1 g, 46.9 mmol) were added to 300 ml of THF under a nitrogen atmosphere, and the mixture was stirred and refluxed. Then, potassium carbonate (19.5 g, 140.7 mmol) was dissolved in 58 ml of water, and then added thereto. Thereafter, it was stirred sufficiently, followed by adding bis(tri-tert-butylphosphine)palladium(0) (0.2 g, 0.5 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 26.7 g of Compound 2-8 (yield 78%, MS: [M+H]+=731).

### Preparation Example 2-9

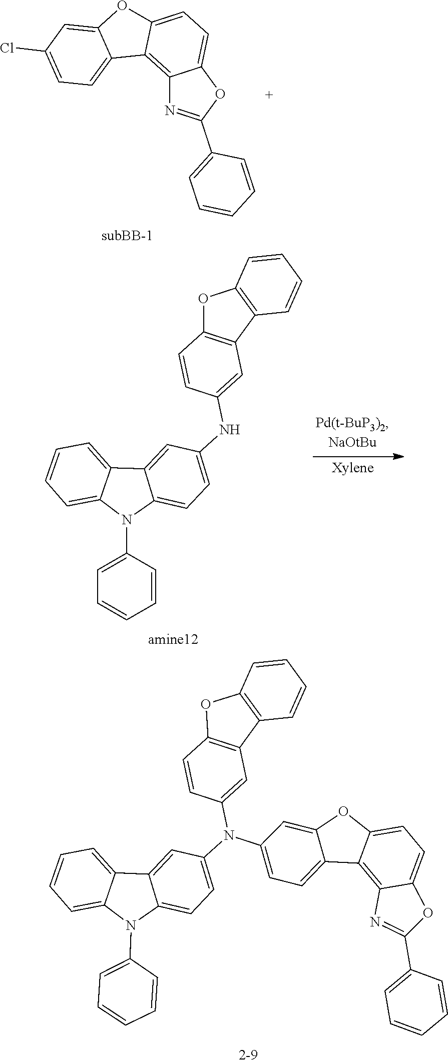

Compound subBB-1 (10 g, 31.3 mmol), Compound amine12 (13.3 g, 31.3 mmol), and sodium tert-butoxide (10 g, 46.9 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 3 hours, the reaction was completed, and the solvent was removed under reduced pressure after cooling to room temperature. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13.2 g of Compound 2-9 (yield 60%, MS: [M+H]+=703).

### Preparation Example 2-10

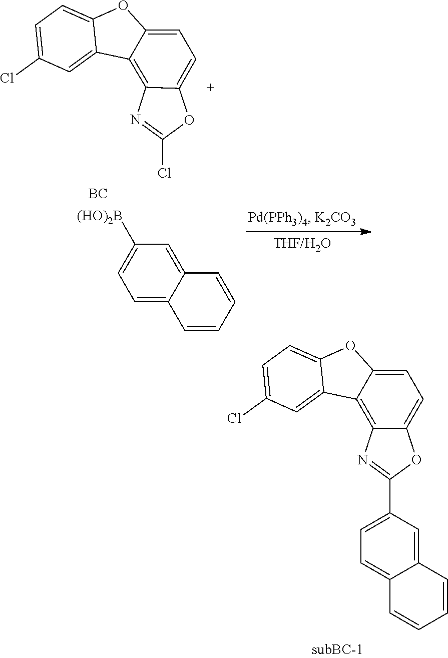 [SMILES: Clc1ccc2oc3ccc4oc(Cl)nc4c3c2c1 ; "BC" ; OB(O)c1ccc2ccccc2c1 ; Clc1ccc2oc3ccc4oc(-c5ccc6ccccc6c5)nc4c3c2c1]

Compound BC (15 g, 53.9 mmol) and naphthalen-2-ylboronic acid (9.3 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)palladium(0) (0.6 g, 0.5 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.1 g of Compound subBC-1 (yield 76%, MS: [M+H]+=370).

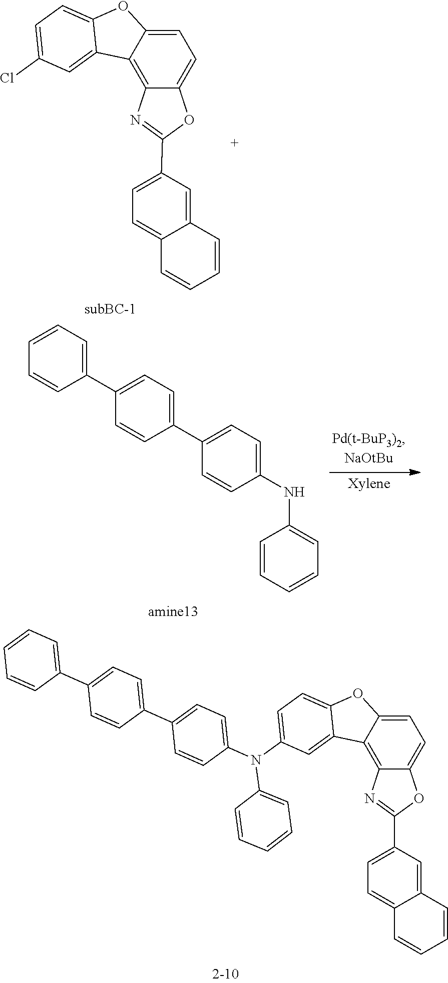 [SMILES: Clc1ccc2oc3ccc4oc(-c5ccc6ccccc6c5)nc4c3c2c1 ; c1ccc(Nc2ccc(-c3ccc(-c4ccccc4)cc3)cc2)cc1 ; c1ccc(-c2ccc(-c3ccc(N(c4ccccc4)c4ccc5oc6ccc7oc(-c8ccc9ccccc9c8)nc7c6c5c4)cc3)cc2)cc1]

Compound subBC-1 (10 g, 27 mmol), Compound amine13 (8.7 g, 27 mmol), and sodium tert-butoxide (8.6 g, 40.6 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 3 hours, the reaction was completed, and the solvent was removed under reduced pressure after cooling to room temperature. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 9 g of Compound 2-10 (yield 51%, MS: [M+H]+=655).

### Preparation Example 2-11

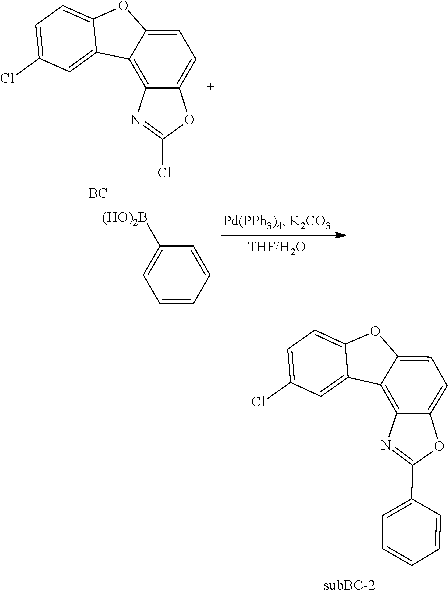 [SMILES: Clc1ccc2oc3ccc4oc(Cl)nc4c3c2c1 ; "BC" ; OB(O)c1ccccc1 ; Clc1ccc2oc3ccc4oc(-c5ccccc5)nc4c3c2c1]

Compound BC (15 g, 53.9 mmol) and phenylboronic acid (6.6 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)palladium(0) (0.6 g, 0.5 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.4 g of Compound subBC-2 (yield 78%, MS: [M+H]+=320).

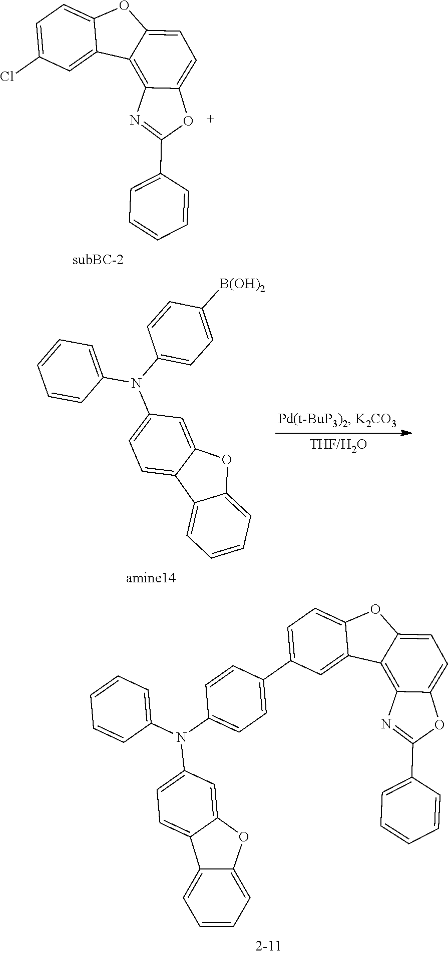 [SMILES: Clc1ccc2oc3ccc4oc(-c5ccccc5)nc4c3c2c1 ; OB(O)c1ccc(N(c2ccccc2)c2ccc3c(c2)oc2ccccc23)cc1 ; c1ccc(-c2nc3c(ccc4oc5ccc(-c6ccc(N(c7ccccc7)c7ccc8c(c7)oc7ccccc78)cc6)cc5c43)o2)cc1]

Compound subBC-2 (15 g, 46.9 mmol) and Compound amine14 (17.8 g, 46.9 mmol) were added to 300 ml of THF under a nitrogen atmosphere, and the mixture was stirred and refluxed. Then, potassium carbonate (19.5 g, 140.7 mmol) was dissolved in 58 ml of water, and then added thereto. Thereafter, it was stirred sufficiently, followed by adding bis(tri-tert-butylphosphine)palladium(0) (0.2 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.5 g of Compound 2-11 (yield 74%, MS: [M+H]+=619).

### Preparation Example 2-12

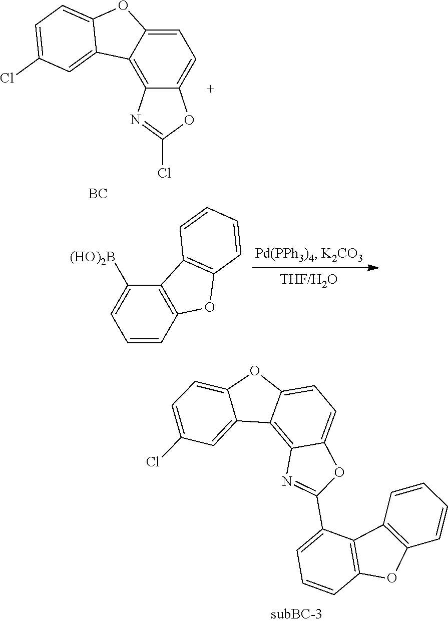 [SMILES: Clc1ccc2oc3ccc4oc(Cl)nc4c3c2c1 ; "BC" ; OB(O)c1cccc2oc3ccccc3c12 ; Clc1ccc2oc3ccc4oc(-c5cccc6oc7ccccc7c56)nc4c3c2c1]

Compound BC (15 g, 53.9 mmol) and dibenzo[b,d]furan-1-ylboronic acid (11.4 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)palladium(0) (0.6 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.9 g of Compound subBC-3 (yield 63%, MS: [M+H]+=410).

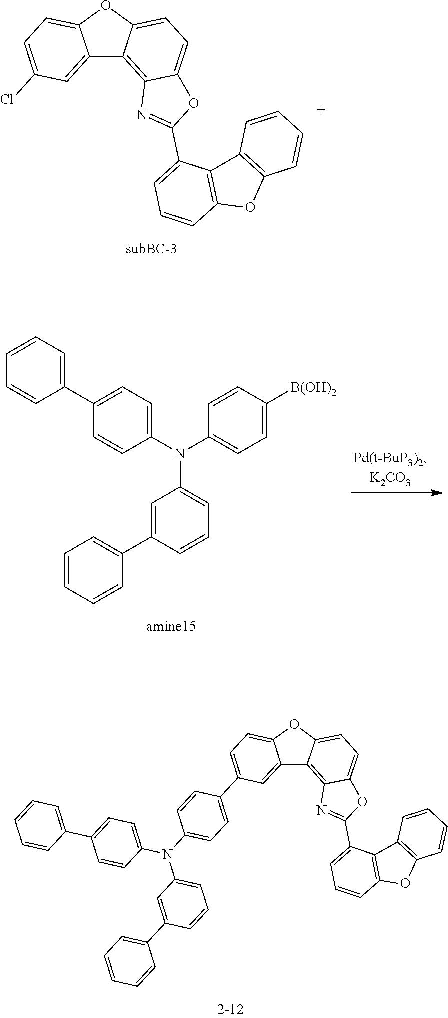 [SMILES: Clc1ccc2oc3ccc4oc(-c5cccc6oc7ccccc7c56)nc4c3c2c1 ; OB(O)c1ccc(N(c2ccc(-c3ccccc3)cc2)c2cccc(-c3ccccc3)c2)cc1 ; c1ccc(-c2ccc(N(c3ccc(-c4ccc5oc6ccc7oc(-c8cccc9oc%10ccccc%10c89)nc7c6c5c4)cc3)c3cccc(-c4ccccc4)c3)cc2)cc1]

Compound subBC-3 (15 g, 36.6 mmol) and Compound amine15 (16.2 g, 36.6 mmol) were added to 300 ml of THF under a nitrogen atmosphere, and the mixture was stirred and refluxed. Then, potassium carbonate (15.2 g, 109.8 mmol) was dissolved in 46 ml of water, and then added thereto. Thereafter, it was stirred sufficiently, followed by adding bis(tri-tert-butylphosphine)palladium(0) (0.2 g, 0.4 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 18.3 g of Compound 2-12 (yield 65%, MS: [M+H]+=771).

### Preparation Example 2-13

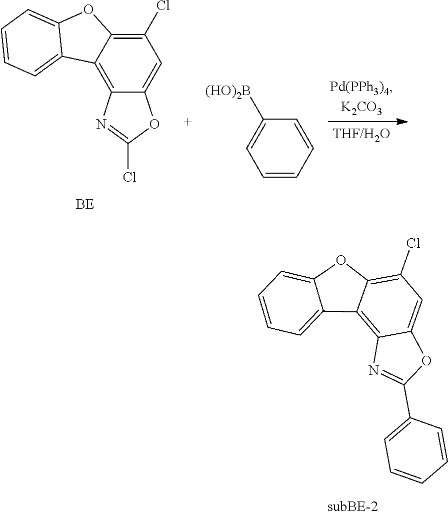 [SMILES: Clc1nc2c(cc(Cl)c3oc4ccccc4c32)o1 ; OB(O)c1ccccc1 ; Clc1cc2oc(-c3ccccc3)nc2c2c1oc1ccccc12]

Compound BE (15 g, 53.9 mmol) and phenylboronic acid (6.6 g, 53.9 mmol) were added to 300 ml of THF under a nitrogen atmosphere, and the mixture was stirred and refluxed. Then, potassium carbonate (22.4 g, 161.8 mmol) was dissolved in 67 ml of water, and then added thereto. Thereafter, it was stirred sufficiently, followed by adding tetrakis(triphenylphosphine)-palladium(0) (0.6 g, 0.5 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.1 g of Compound subBE-2 (yield 76%, MS: [M+H]+=320).

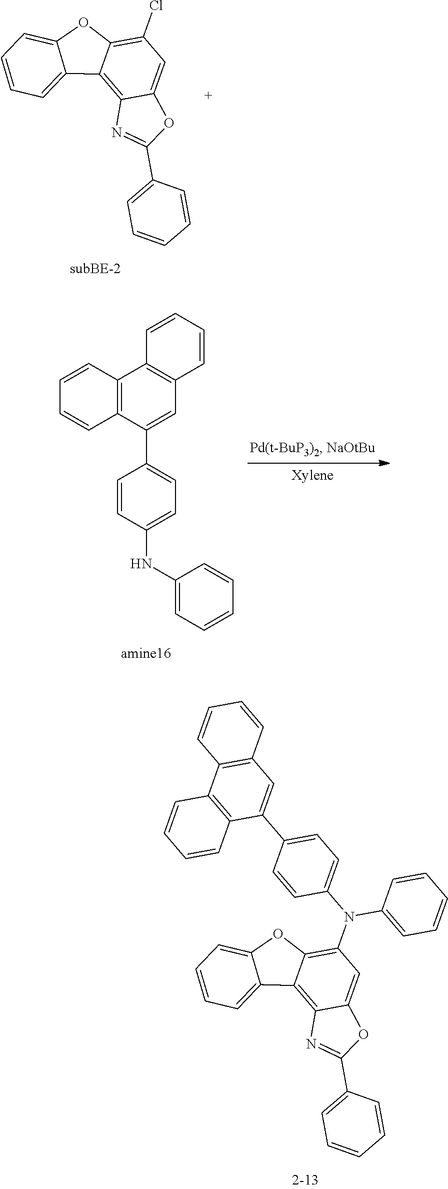 [SMILES: Clc1cc2oc(-c3ccccc3)nc2c2c1oc1ccccc12 ; c1ccc(Nc2ccc(-c3cc4ccccc4c4ccccc34)cc2)cc1 ; c1ccc(-c2nc3c(cc(N(c4ccccc4)c4ccc(-c5cc6ccccc6c6ccccc56)cc4)c4oc5ccccc5c43)o2)cc1]

Compound subBE-2 (10 g, 31.3 mmol), Compound amine16 (10.8 g, 31.3 mmol), and sodium tert-butoxide (10 g, 46.9 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 2 hours, the reaction was completed, and the solvent was removed under reduced pressure after cooling to room temperature. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 13 g of Compound 2-13 (yield 66%, MS: [M+H]+=629).

### Preparation Example 2-14

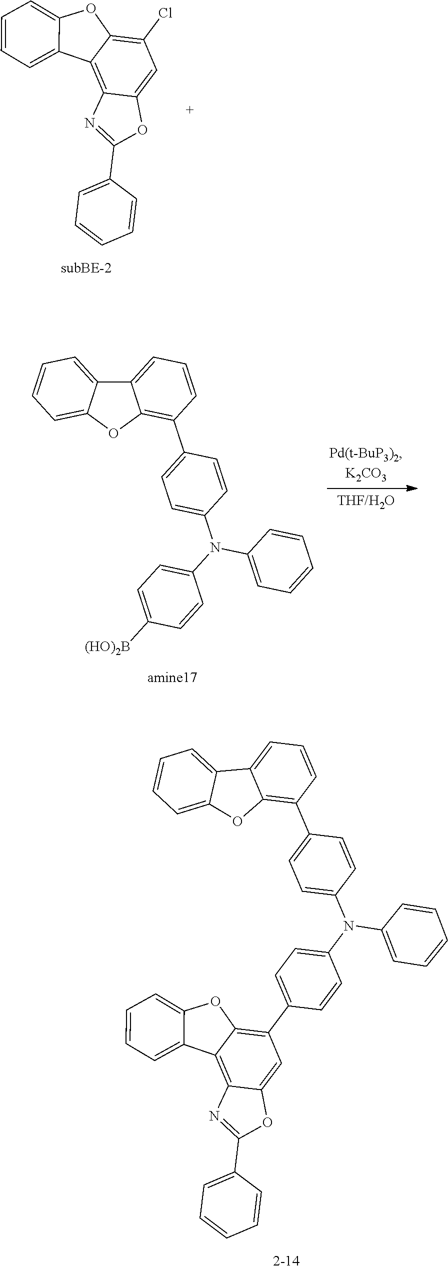 [SMILES: Clc1cc2oc(-c3ccccc3)nc2c2c1oc1ccccc12 ; OB(O)c1ccc(N(c2ccccc2)c2ccc(-c3cccc4c3oc3ccccc34)cc2)cc1 ; c1ccc(-c2nc3c(cc(-c4ccc(N(c5ccccc5)c5ccc(-c6cccc7c6oc6ccccc67)cc5)cc4)c4oc5ccccc5c43)o2)cc1]

Compound subBE-2 (15 g, 46.9 mmol) and Compound amine17 (21.4 g, 46.9 mmol) were added to 300 ml of THF under a nitrogen atmosphere, and the mixture was stirred and refluxed. Then, potassium carbonate (19.5 g, 140.7 mmol) was dissolved in 58 ml of water, and then added thereto. Thereafter, it was stirred sufficiently, followed by adding bis(tri-tert-butylphosphine)palladium(0) (0.2 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.4 g of Compound 2-14 (yield 72%, MS: [M+H]+=695).

### Preparation Example 2-15

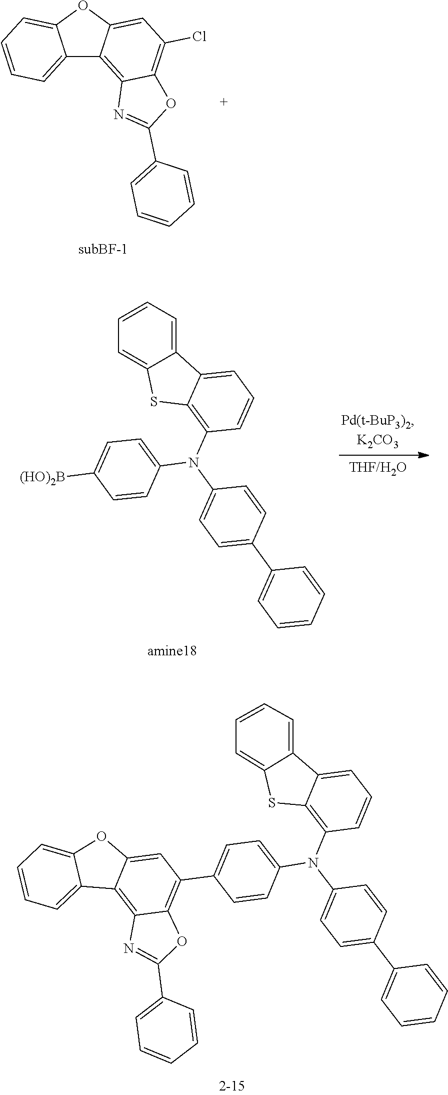 [SMILES: Clc1cc2oc3ccccc3c2c2nc(-c3ccccc3)oc12 ; OB(O)c1ccc(N(c2ccc(-c3ccccc3)cc2)c2cccc3c2sc2ccccc23)cc1 ; c1ccc(-c2ccc(N(c3ccc(-c4cc5oc6ccccc6c5c5nc(-c6ccccc6)oc45)cc3)c3cccc4c3sc3ccccc34)cc2)cc1]

Compound subBF-1 (15 g, 46.9 mmol) and Compound amine18 (22.1 g, 46.9 mmol) were added to 300 ml of THF under a nitrogen atmosphere, and the mixture was stirred and refluxed. Then, potassium carbonate (19.5 g, 140.7 mmol) was dissolved in 58 ml of water, and then added thereto. Thereafter, it was stirred sufficiently, followed by adding bis(tri-tert-butylphosphine)palladium(0) (0.2 g, 0.5 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 23.3 g of Compound 2-15 (yield 70%, MS: [M+H]+=711).

### Preparation Example 2-16

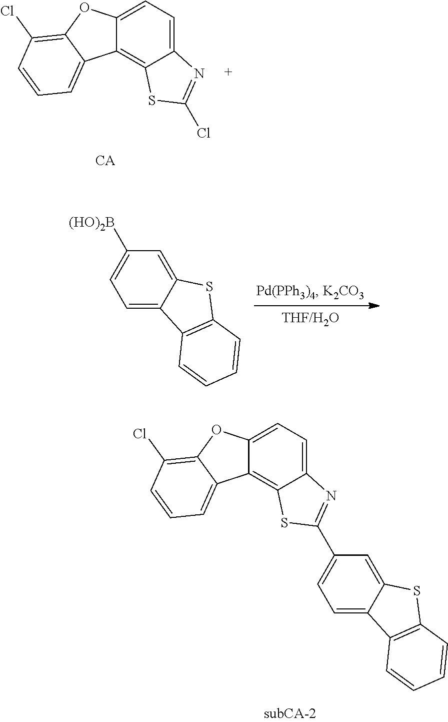

Compound CA (15 g, 51 mmol) and dibenzo[b,d]thiophen-3-ylboronic acid (11.6 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 14.4 g of Compound subCA-2 (yield 64%, MS: [M+H]+=442).

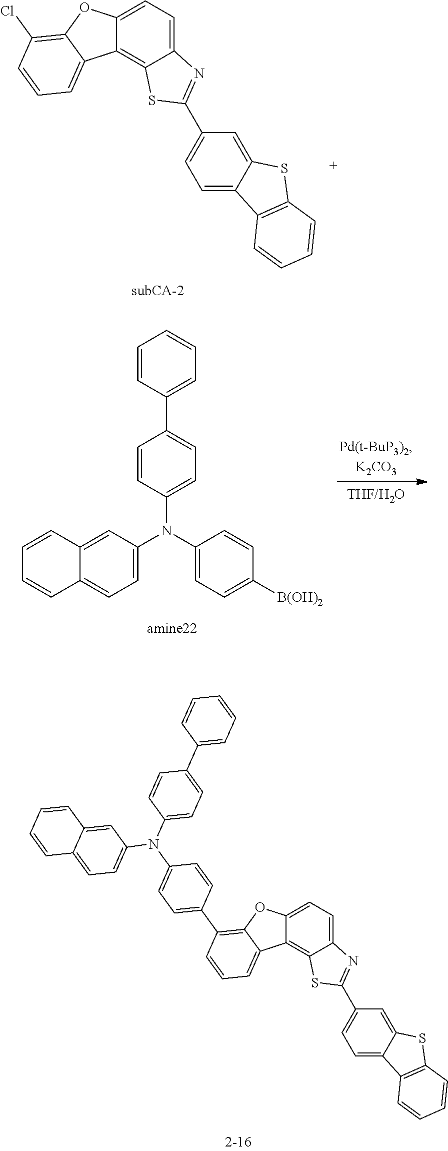 [SMILES: Clc1cccc2c1oc1ccc3nc(-c4ccc5c(c4)sc4ccccc45)sc3c12 ; OB(O)c1ccc(N(c2ccc(-c3ccccc3)cc2)c2ccc3ccccc3c2)cc1 ; c1ccc(-c2ccc(N(c3ccc(-c4cccc5c4oc4ccc6nc(-c7ccc8c(c7)sc7ccccc78)sc6c45)cc3)c3ccc4ccccc4c3)cc2)cc1]

Compound subCA-2 (15 g, 33.9 mmol) and Compound amine22 (14.1 g, 33.9 mmol) were added to 300 ml of THF under a nitrogen atmosphere, and the mixture was stirred and refluxed. Then, potassium carbonate (14.1 g, 101.8 mmol) was dissolved in 42 ml of water, and then added thereto. Thereafter, it was stirred sufficiently, followed by adding bis(tri-tert-butylphosphine)palladium(0) (0.2 g, 0.3 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.8 g of Compound 2-16 (yield 79%, MS: [M+H]+=777).

### Preparation Example 2-17

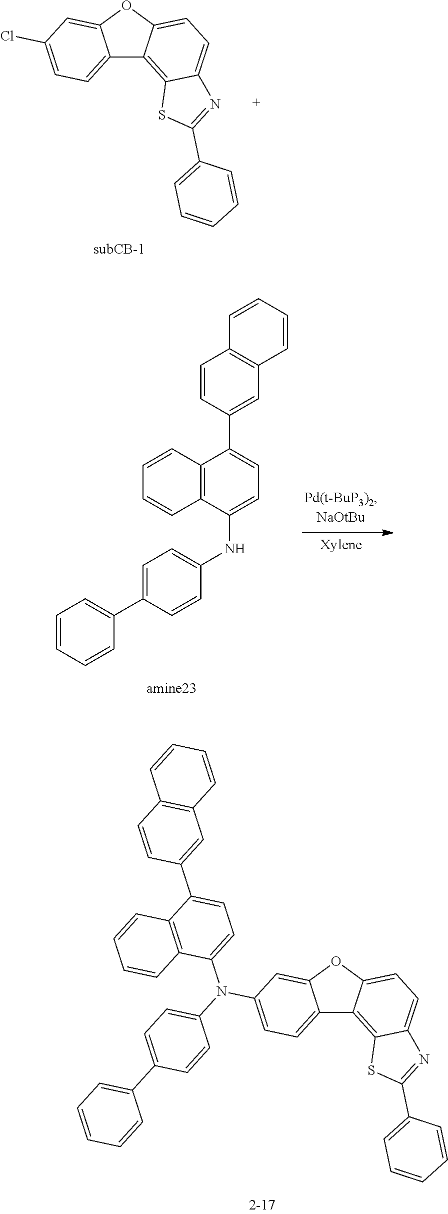 [SMILES: Clc1ccc2c(c1)oc1ccc3nc(-c4ccccc4)sc3c12 ; c1ccc(-c2ccc(Nc3ccc(-c4ccc5ccccc5c4)c4ccccc34)cc2)cc1 ; c1ccc(-c2ccc(N(c3ccc4c(c3)oc3ccc5nc(-c6ccccc6)sc5c34)c3ccc(-c4ccc5ccccc5c4)c4ccccc34)cc2)cc1]

Compound subCB-1 (10 g, 29.8 mmol), Compound amine23 (12.6 g, 29.8 mmol), and sodium tert-butoxide (9.5 g, 44.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 3 hours, the reaction was completed, and the solvent was removed under reduced pressure after cooling to room temperature. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12.5 g of Compound 2-17 (yield 58%, MS: [M+H]+=722).

### Preparation Example 2-18

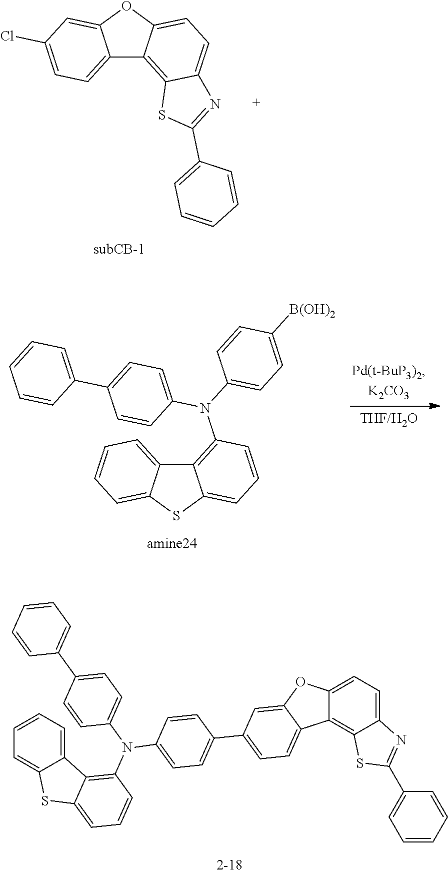 [SMILES: Clc1ccc2c(c1)oc1ccc3nc(-c4ccccc4)sc3c12 ; OB(O)c1ccc(N(c2ccc(-c3ccccc3)cc2)c2cccc3sc4ccccc4c23)cc1 ; c1ccc(-c2ccc(N(c3ccc(-c4ccc5c(c4)oc4ccc6nc(-c7ccccc7)sc6c45)cc3)c3cccc4sc5ccccc5c34)cc2)cc1]

Compound subCB-1 (15 g, 44.7 mmol) and Compound amine24 (21.1 g, 44.7 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.1 g of Compound 2-18 (yield 62%, MS: [M+H]+=727).

### Preparation Example 2-19

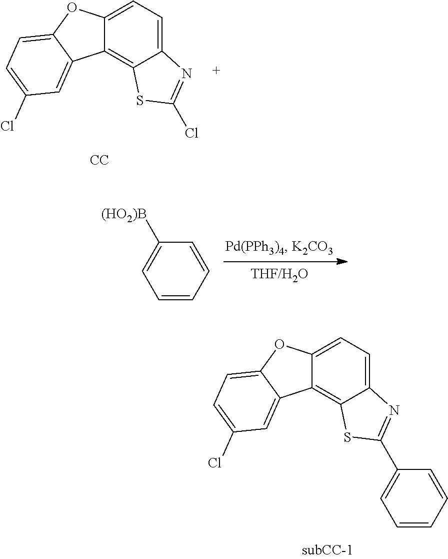

Compound CC (15 g, 51 mmol) and phenylboronic acid (6.2 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)-palladium(0) (0.6 g, 0.5 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 10.9 g of Compound subCC-1 (yield 64%, MS: [M+H]+=336).

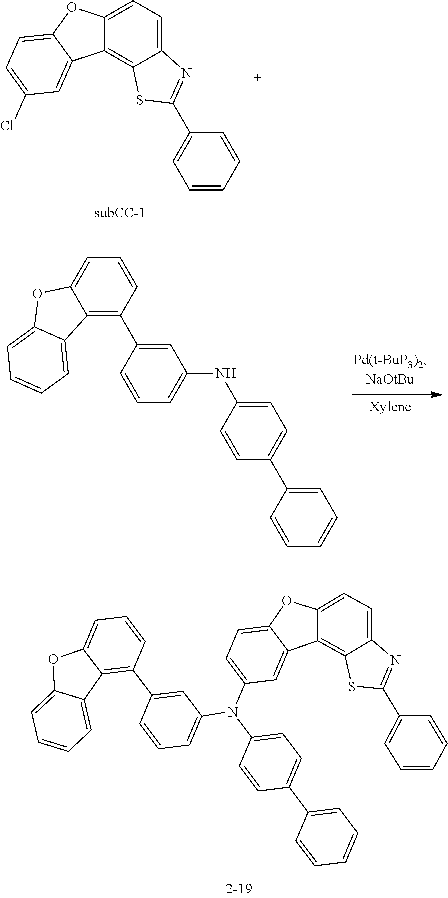 [SMILES: Clc1ccc2oc3ccc4nc(-c5ccccc5)sc4c3c2c1 ; c1ccc(-c2ccc(Nc3cccc(-c4cccc5oc6ccccc6c45)c3)cc2)cc1 ; c1ccc(-c2ccc(N(c3cccc(-c4cccc5oc6ccccc6c45)c3)c3ccc4oc5ccc6nc(-c7ccccc7)sc6c5c4c3)cc2)cc1]

Compound subCC-1 (10 g, 29.8 mmol), Compound amine25 (12.3 g, 29.8 mmol), and sodium tert-butoxide (9.5 g, 44.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 3 hours, the reaction was completed, and the solvent was removed under reduced pressure after cooling to room temperature. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 14 g of Compound 2-19 (yield 66%, MS: [M+H]+=711).

### Preparation Example 2-20

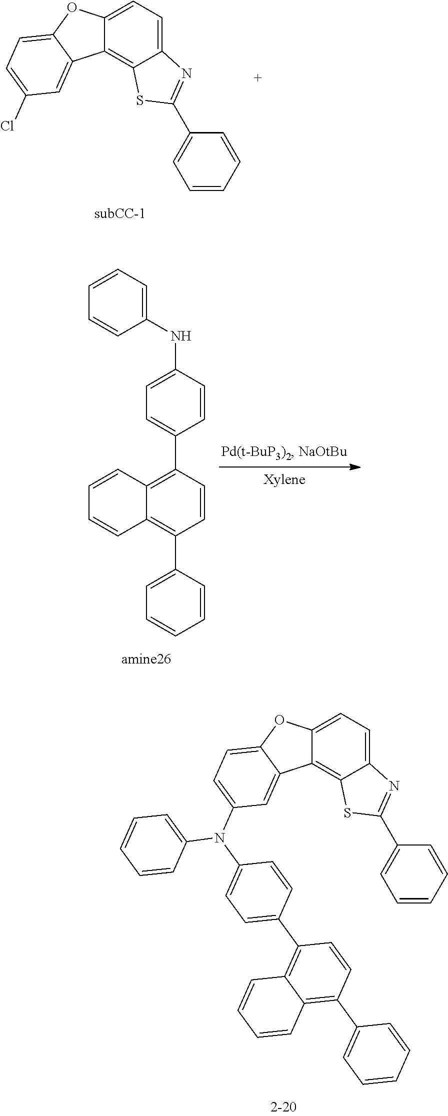 [SMILES: Clc1ccc2oc3ccc4nc(-c5ccccc5)sc4c3c2c1 ; c1ccc(Nc2ccc(-c3ccc(-c4ccccc4)c4ccccc34)cc2)cc1 ; c1ccc(-c2nc3ccc4oc5ccc(N(c6ccccc6)c6ccc(-c7ccc(-c8ccccc8)c8ccccc78)cc6)cc5c4c3s2)cc1]

Compound subCC-1 (10 g, 29.8 mmol), Compound amine26 (11.1 g, 29.8 mmol), and sodium tert-butoxide (9.5 g, 44.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 3 hours, the reaction was completed, and the solvent was removed under reduced pressure after cooling to room temperature. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12 g of Compound 2-20 (yield 60%, MS: [M+H]+=671).

### Preparation Example 2-21

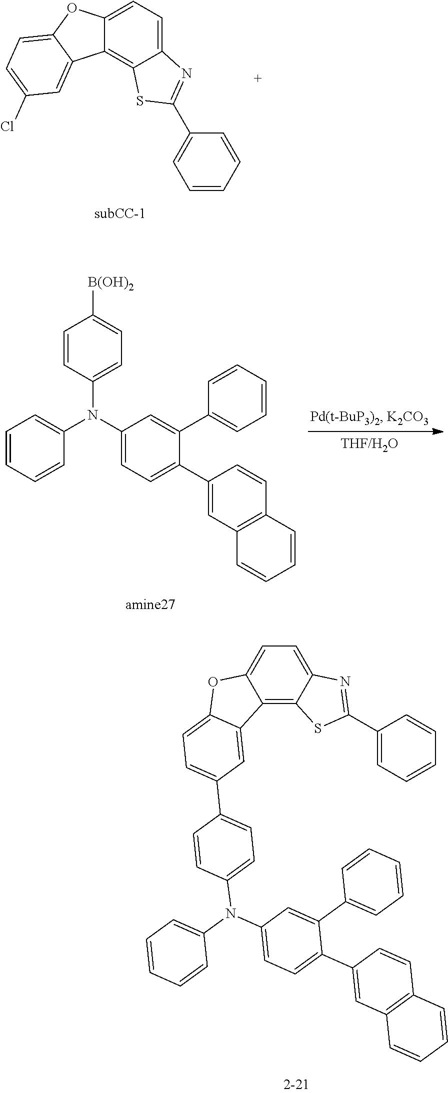 [SMILES: Clc1ccc2oc3ccc4nc(-c5ccccc5)sc4c3c2c1 ; OB(O)c1ccc(N(c2ccccc2)c2ccc(-c3ccc4ccccc4c3)c(-c3ccccc3)c2)cc1 ; c1ccc(-c2nc3ccc4oc5ccc(-c6ccc(N(c7ccccc7)c7ccc(-c8ccc9ccccc9c8)c(-c8ccccc8)c7)cc6)cc5c4c3s2)cc1]

Compound subCC-1 (10 g, 29.8 mmol) and Compound amine27 (14.6 g, 29.8 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.3 mmol). After 9 hours, cooling was performed to room temperature. Then, the organic layer was separated from the water layer, and then the organic layer was distilled. Then, this was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.8 g of Compound 2-21 (yield 53%, MS: [M+H]+=747).

### Preparation Example 2-22

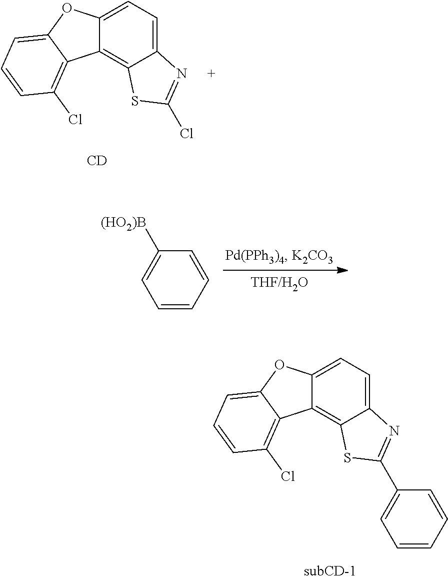

Compound CD (15 g, 51 mmol) and phenylboronic acid (6.2 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)-palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.8 g of Compound subCD-1 (yield 75%, MS: [M+H]+=336).

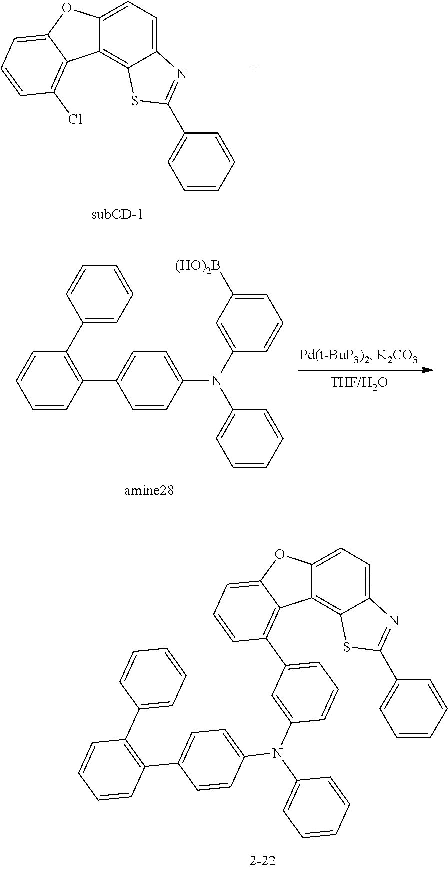 [SMILES: Clc1cccc2oc3ccc4nc(-c5ccccc5)sc4c3c12 ; OB(O)c1cccc(N(c2ccccc2)c2ccc(-c3ccccc3-c3ccccc3)cc2)c1 ; c1ccc(-c2nc3ccc4oc5cccc(-c6cccc(N(c7ccccc7)c7ccc(-c8ccccc8-c8ccccc8)cc7)c6)c5c4c3s2)cc1]

Compound subCD-1 (15 g, 44.7 mmol) and Compound amine28 (19.7 g, 44.7 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 11 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 20.2 g of Compound 2-22 (yield 65%, MS: [M+H]+=697).

### Preparation Example 2-23

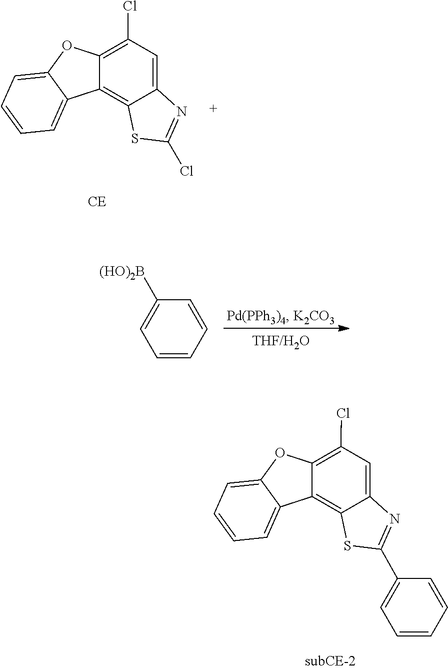 [SMILES: Clc1nc2cc(Cl)c3oc4ccccc4c3c2s1 ; OB(O)c1ccccc1 ; Clc1cc2nc(-c3ccccc3)sc2c2c1oc1ccccc12]

Compound CE (15 g, 51 mmol) and phenylboronic acid (6.2 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.5 g of Compound subCE-2 (yield 79%, MS: [M+H]+=336).

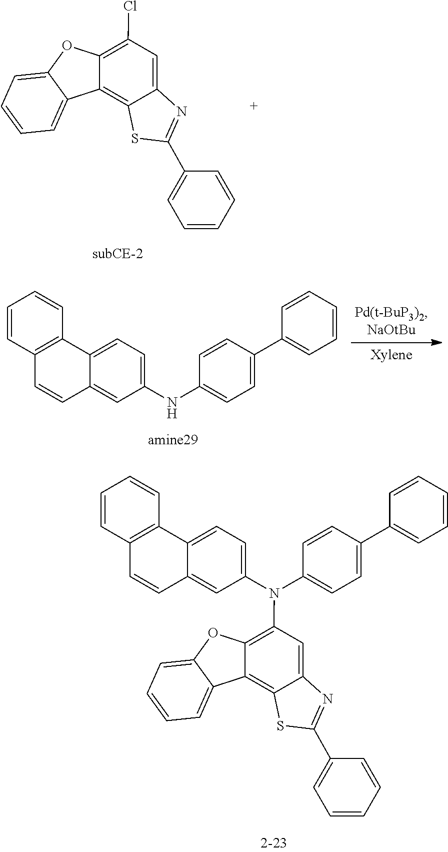 [SMILES: Clc1cc2nc(-c3ccccc3)sc2c2c1oc1ccccc12 ; c1ccc(-c2ccc(Nc3ccc4c(ccc5ccccc54)c3)cc2)cc1 ; c1ccc(-c2ccc(N(c3ccc4c(ccc5ccccc54)c3)c3cc4nc(-c5ccccc5)sc4c4c3oc3ccccc34)cc2)cc1]

Compound subCE-2 (10 g, 29.8 mmol), Compound amine29 (10.3 g, 29.8 mmol), and sodium tert-butoxide (9.5 g, 44.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 2 hours, the reaction was completed, and the solvent was removed under reduced pressure after cooling to room temperature. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 11.9 g of Compound 2-23 (yield 62%, MS: [M+H]+=645).

### Preparation Example 2-24

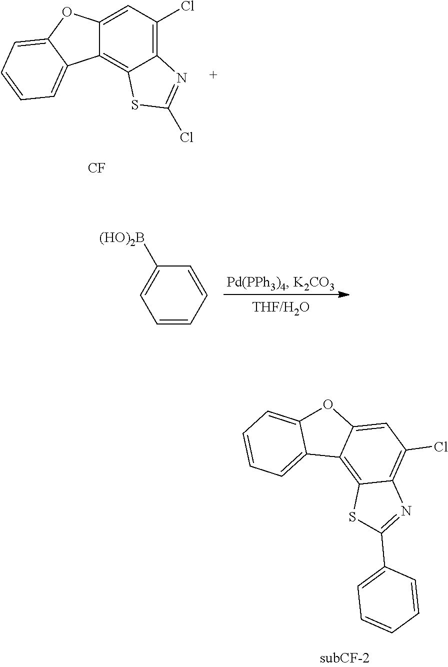 [SMILES: Clc1nc2c(Cl)cc3oc4ccccc4c3c2s1 ; "CF" ; OB(O)c1ccccc1 ; Clc1cc2oc3ccccc3c2c2sc(-c3ccccc3)nc12]

Compound CF (15 g, 51 mmol) and phenylboronic acid (6.2 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.6 g of Compound subCF-2 (yield 68%, MS: [M+H]+=336).

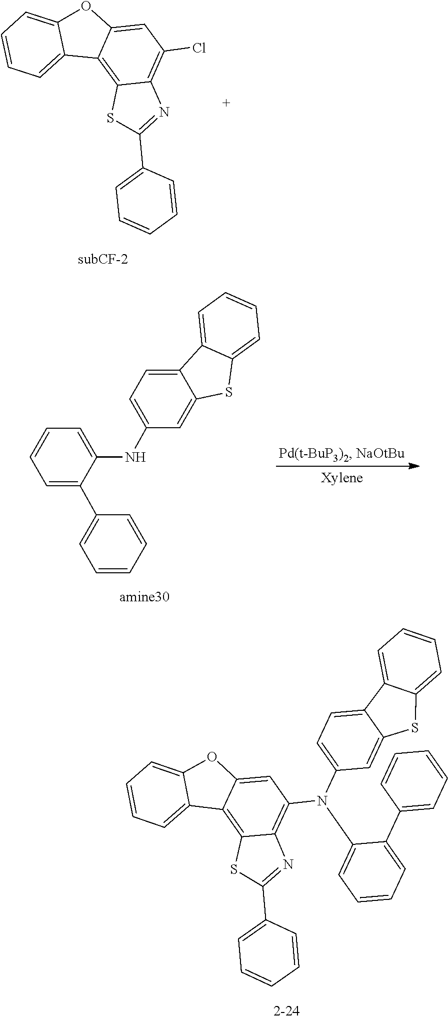 [SMILES: Clc1cc2oc3ccccc3c2c2sc(-c3ccccc3)nc12 ; c1ccc(-c2ccccc2Nc2ccc3c(c2)sc2ccccc23)cc1 ; c1ccc(-c2nc3c(N(c4ccc5c(c4)sc4ccccc45)c4ccccc4-c4ccccc4)cc4oc5ccccc5c4c3s2)cc1]

Compound subCF-2 (10 g, 29.8 mmol), Compound amine30 (10.5 g, 29.8 mmol), and sodium tert-butoxide (9.5 g, 44.7 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 3 hours, the reaction was completed, and the solvent was removed under reduced pressure after cooling to room temperature. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 12.4 g of Compound 2-24 (yield 64%, MS: [M+H]+=651).

### Preparation Example 2-25

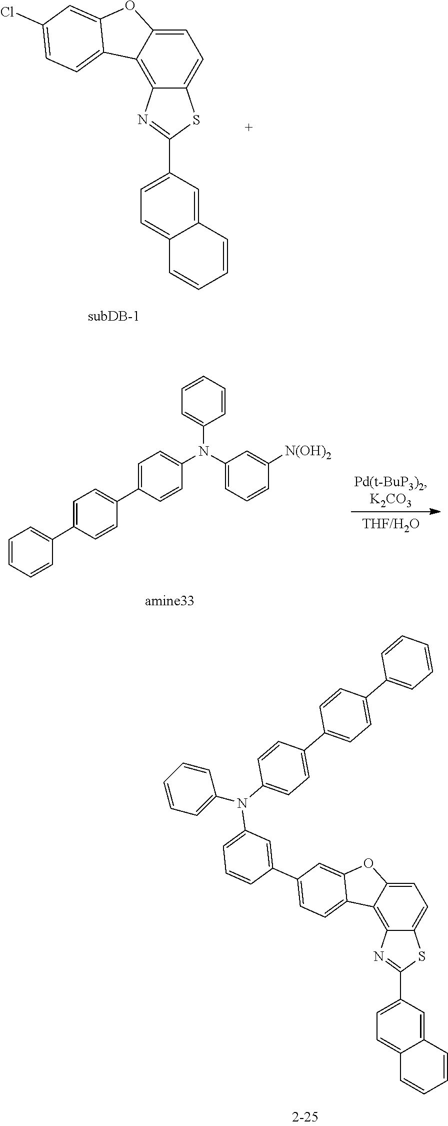

Compound subDB-1 (15 g, 38.9 mmol) and Compound amine33 (17.2 g, 38.9 mmol) were added to 300 ml of THF under a nitrogen atmosphere, and the mixture was stirred and refluxed. Then, potassium carbonate (16.1 g, 116.6 mmol) was dissolved in 48 ml of water, and then added thereto. Thereafter, it was stirred sufficiently, followed by adding bis(tri-tert-butylphosphine)palladium(0) (0.2 g, 0.4 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21.2 g of Compound 2-25 (yield 73%, MS: [M+H]+=747).

### Preparation Example 2-26

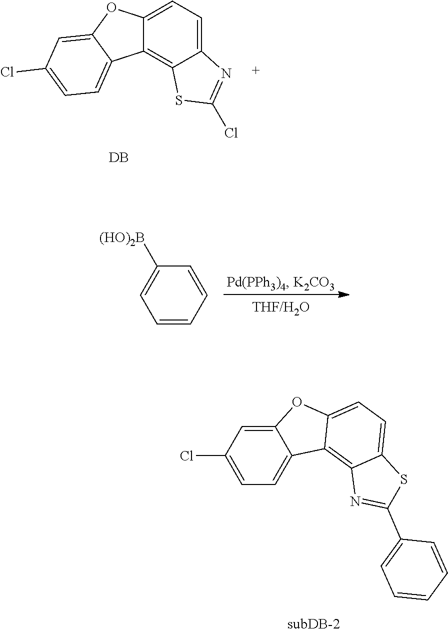 [SMILES: Clc1ccc2c(c1)oc1ccc3nc(Cl)sc3c12 ; "DB" ; OB(O)c1ccccc1 ; Clc1ccc2c(c1)oc1ccc3sc(-c4ccccc4)nc3c12]

Compound DB (15 g, 51 mmol) and phenylboronic acid (6.2 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 10 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 13.2 g of Compound subDB-2 (yield 77%, MS: [M+H]+=336).

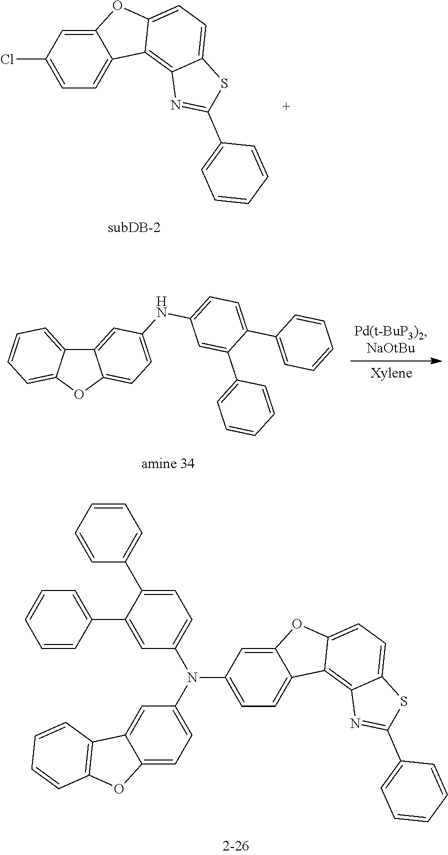 [SMILES: Clc1ccc2c(c1)oc1ccc3sc(-c4ccccc4)nc3c12 ; c1ccc(-c2ccc(Nc3ccc4oc5ccccc5c4c3)cc2-c2ccccc2)cc1 ; c1ccc(-c2nc3c(ccc4oc5cc(N(c6ccc(-c7ccccc7)c(-c7ccccc7)c6)c6ccc7oc8ccccc8c7c6)ccc5c43)s2)cc1]

Compound subDB-2 (10 g, 31.3 mmol), Compound amine34 (12.9 g, 31.3 mmol), and sodium tert-butoxide (10 g, 46.9 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After 2 hours, the reaction was completed, and the solvent was removed under reduced pressure after cooling to room temperature. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 15 g of Compound 2-26 (yield 69%, MS: [M+H]+=695).

### Preparation Example 2-27

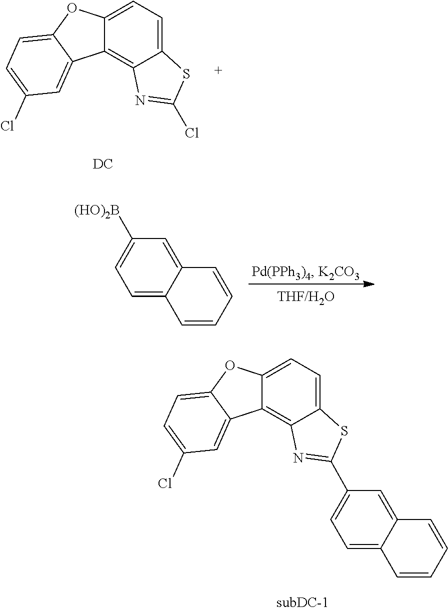

Compound DC (15 g, 51 mmol) and naphthalen-2-ylboronic acid (8.8 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)-palladium(0) (0.6 g, 0.5 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 12.8 g of Compound subDC-1 (yield 65%, MS: [M+H]+=386).

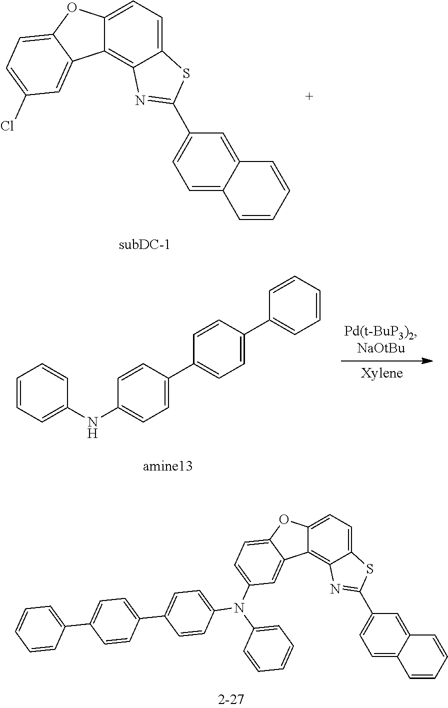 [SMILES: Clc1ccc2oc3ccc4sc(-c5ccc6ccccc6c5)nc4c3c2c1 ; c1ccc(Nc2ccc(-c3ccc(-c4ccccc4)cc3)cc2)cc1 ; c1ccc(-c2ccc(-c3ccc(N(c4ccccc4)c4ccc5oc6ccc7sc(-c8ccc9ccccc9c8)nc7c6c5c4)cc3)cc2)cc1]

Compound subDC-1 (10 g, 25.9 mmol), Compound amine13 (8.3 g, 25.9 mmol), and sodium tert-butoxide (8.3 g, 38.9 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After 2 hours, the reaction was completed, and the solvent was removed under reduced pressure after cooling to room temperature. Then, the compound was completely dissolved again in chloroform, and washed twice with water. Thereafter, the organic layer was separated, treated with anhydrous magnesium sulfate, then filtered, and the filtrate was distilled under reduced pressure. The concentrated compound was purified by silica gel column chromatography to prepare 10.9 g of Compound 2-27 (yield 63%, MS: [M+H]+=671).

### Preparation Example 2-28

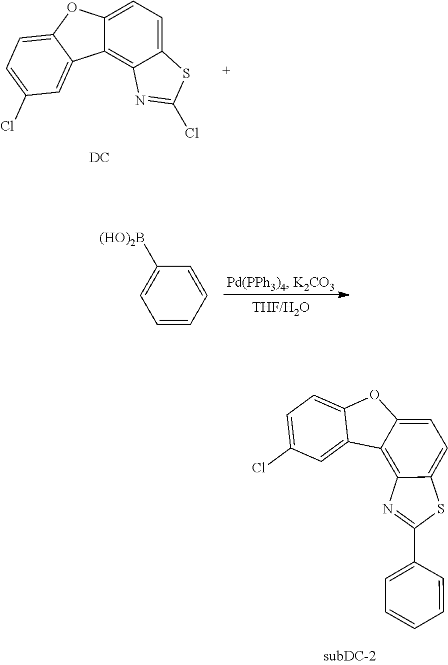 [SMILES: Clc1ccc2oc3ccc4sc(Cl)nc4c3c2c1 ; "DC" ; OB(O)c1ccccc1 ; Clc1ccc2oc3ccc4sc(-c5ccccc5)nc4c3c2c1]

Compound DC (15 g, 51 mmol) and phenylboronic acid (6.2 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)-palladium(0) (0.6 g, 0.5 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 11.1 g of Compound subDC-2 (yield 65%, MS: [M+H]+=336).

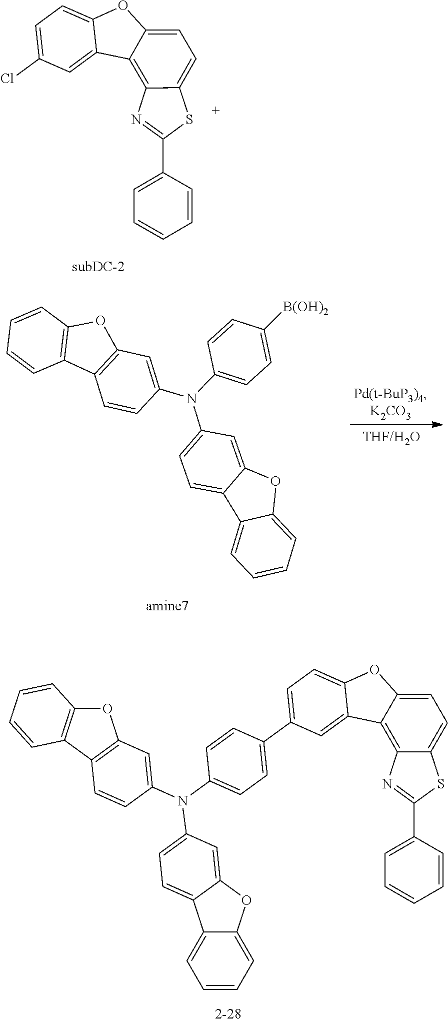 [SMILES: Clc1ccc2oc3ccc4sc(-c5ccccc5)nc4c3c2c1 ; OB(O)c1ccc(N(c2ccc3c(c2)oc2ccccc23)c2ccc3c(c2)oc2ccccc23)cc1 ; c1ccc(-c2nc3c(ccc4oc5ccc(-c6ccc(N(c7ccc8c(c7)oc7ccccc78)c7ccc8c(c7)oc7ccccc78)cc6)cc5c43)s2)cc1]

Compound subDC-2 (15 g, 44.7 mmol) and Compound amine7 (21 g, 44.7 mmol) were added to 300 ml of THF under a nitrogen atmosphere, and the mixture was stirred and refluxed. Then, potassium carbonate (18.5 g, 134 mmol) was dissolved in 56 ml of water, and then added thereto. Thereafter, it was stirred sufficiently, followed by adding bis(tri-tert-butylphosphine)palladium(0) (0.2 g, 0.4 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 21 g of Compound 2-28 (yield 65%, MS: [M+H]+=725).

### Preparation Example 2-29

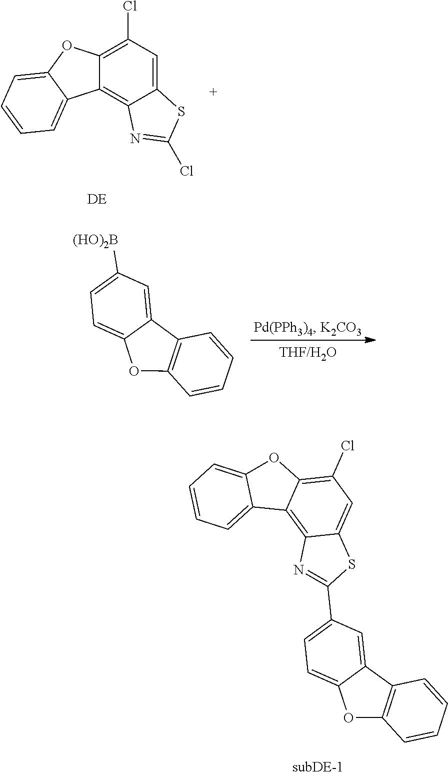 [SMILES: Clc1nc2c(cc(Cl)c3oc4ccccc4c32)s1 ; OB(O)c1ccc2oc3ccccc3c2c1 ; Clc1cc2sc(-c3ccc4oc5ccccc5c4c3)nc2c2c1oc1ccccc12]

Compound DE (15 g, 51 mmol) and dibenzo[b,d]furan-2-ylboronic acid (10.8 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 16 g of Compound subDE-1 (yield 74%, MS: [M+H]+=426).

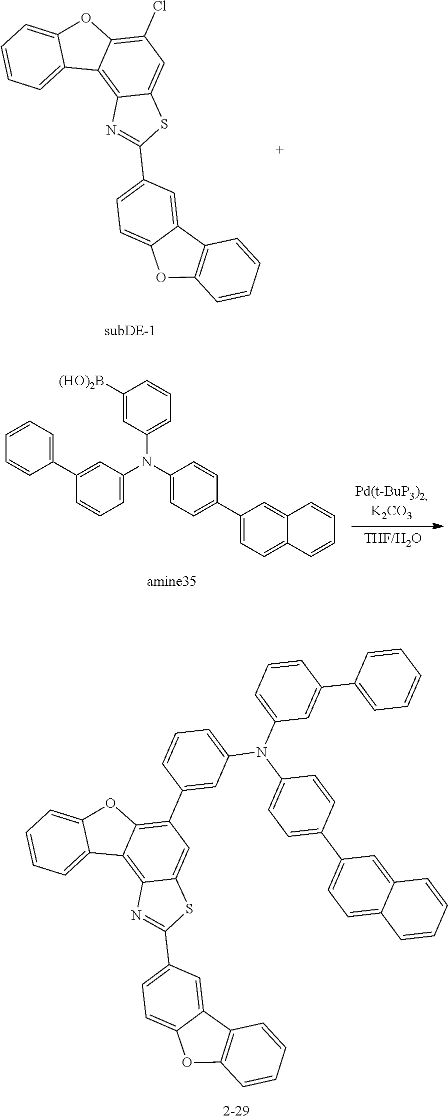 [SMILES: Clc1cc2sc(-c3ccc4oc5ccccc5c4c3)nc2c2c1oc1ccccc12 ; OB(O)c1cccc(N(c2ccc(-c3ccc4ccccc4c3)cc2)c2cccc(-c3ccccc3)c2)c1 ; c1ccc(-c2cccc(N(c3ccc(-c4ccc5ccccc5c4)cc3)c3cccc(-c4cc5sc(-c6ccc7oc8ccccc8c7c6)nc5c5c4oc4ccccc45)c3)c2)cc1]

Compound subDE-1 (15 g, 35.2 mmol) and Compound amine35 (17.3 g, 35.2 mmol) were added to 300 ml of THF under a nitrogen atmosphere, and the mixture was stirred and refluxed. Then, potassium carbonate (14.6 g, 105.7 mmol) was dissolved in 44 ml of water, and then added thereto. Thereafter, it was stirred sufficiently, followed by adding bis(tri-tert-butylphosphine)palladium(0) (0.2 g, 0.4 mmol). After 9 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 19.7 g of Compound 2-29 (yield 67%, MS: [M+H]+=837).

### Preparation Example 2-30

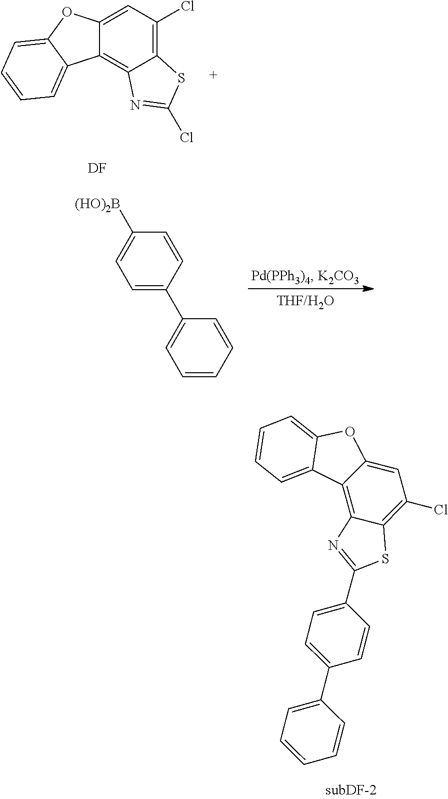 [SMILES: Clc1nc2c(s1)c(Cl)cc1oc3ccccc3c12 ; "DF" ; OB(O)c1ccc(-c2ccccc2)cc1 ; Clc1cc2oc3ccccc3c2c2nc(-c3ccc(-c4ccccc4)cc3)sc12]

Compound DF (15 g, 51 mmol) and [1,1′-biphenyl]-4-ylboronic acid (10.1 g, 51 mmol) were added to 300 ml of THF under a nitrogen atmosphere, and the mixture was stirred and refluxed. Then, potassium carbonate (21.1 g, 153 mmol) was dissolved in 63 ml of water, and then added thereto. Thereafter, it was stirred sufficiently, followed by adding tetrakis(triphenylphosphine)palladium(0) (0.6 g, 0.5 mmol). After 12 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 15.5 g of Compound subDF-2 (yield 74%, MS: [M+H]+=412).

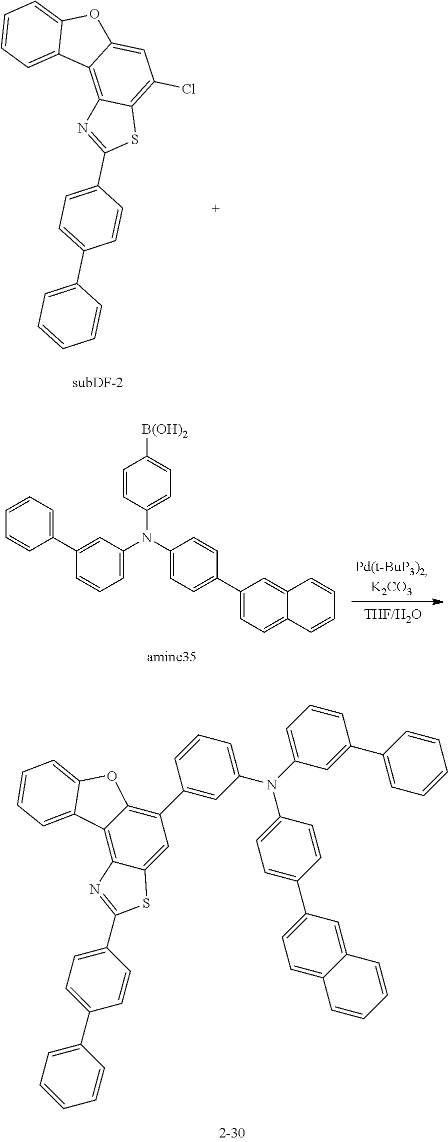 [SMILES: Clc1cc2oc3ccccc3c2c2nc(-c3ccc(-c4ccccc4)cc3)sc12 ; OB(O)c1ccc(N(c2ccc(-c3ccc4ccccc4c3)cc2)c2cccc(-c3ccccc3)c2)cc1 ; c1ccc(-c2ccc(-c3nc4c(cc(-c5cccc(N(c6ccc(-c7ccc8ccccc8c7)cc6)c6cccc(-c7ccccc7)c6)c5)c5oc6ccccc6c54)s3)cc2)cc1]

Compound subDF-2 (15 g, 57.8 mmol) and Compound amine35 (28.4 g, 57.8 mmol) were added to 300 ml of THF under a nitrogen atmosphere, and the mixture was stirred and refluxed. Then, potassium carbonate (23.9 g, 173.3 mmol) was dissolved in 72 ml of water, and then added thereto. Thereafter, it was stirred sufficiently, followed by adding bis(tri-tert-butylphosphine)palladium(0) (0.3 g, 0.6 mmol). After 8 hours of reaction, cooling was performed to room temperature. Then, the organic layer was separated from the water layer, and then the organic layer was distilled. Then, this was dissolved again in chloroform, and washed twice with water. Thereafter, the organic layer was separated, treated with anhydrous magnesium sulfate, stirred, then filtered, and the filtrate was distilled under reduced pressure. The concentrated compound was purified by silica gel column chromatography to prepare 29.4 g of Compound 2-30 (yield 62%, MS: [M+H]+=823).

### EXAMPLES

### Comparative Example A

A glass substrate on which ITO (indium tin oxide) was coated as a thin film to a thickness of 1,000 Å was put into distilled water in which a detergent was dissolved, and ultrasonically cleaned. At this time, a product manufactured by Fischer Co. was used as the detergent, and distilled water filtered twice using a filter manufactured by Millipore Co. was used as the distilled water. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. The substrate was cleaned for 5 minutes using oxygen plasma and then transferred to a vacuum depositor.

A hole injection layer was formed on the prepared ITO transparent electrode to a thickness of 1150 Å with the following Compound HI-1, and the following Compound A-1 was p-doped at a concentration of 1.5 wt %. Then, the following Compound HT-1 was vacuum-deposited on the hole injection layer to a thickness of 800 Å to form a hole transport layer. Thereafter, the following Compound EB-1 was vacuum-deposited on the hole transport layer to a thickness of 150 Å as an electron blocking layer. Then, the following Compound RH-1, and the following Compound Dp-7 were vacuum-deposited on the EB-1 deposited film to a thickness of 400 Å in a weight ratio of 98:2 to form a red light emitting layer. A hole blocking layer was formed by vacuum-depositing the following Compound HB-1 to a thickness of 30 Å on the light emitting layer. Then, the following Compound ET-1 and the following Compound LiQ were vacuum-deposited on the hole blocking layer to a thickness of 300 Å in a weight ratio of 2:1 to form an electron injection and transport layer. Lithium fluoride (LiF) and aluminum were sequentially deposited on the electron injection and transport layer to a thickness of 12 Å and 1,000 Å, respectively, to form a cathode.

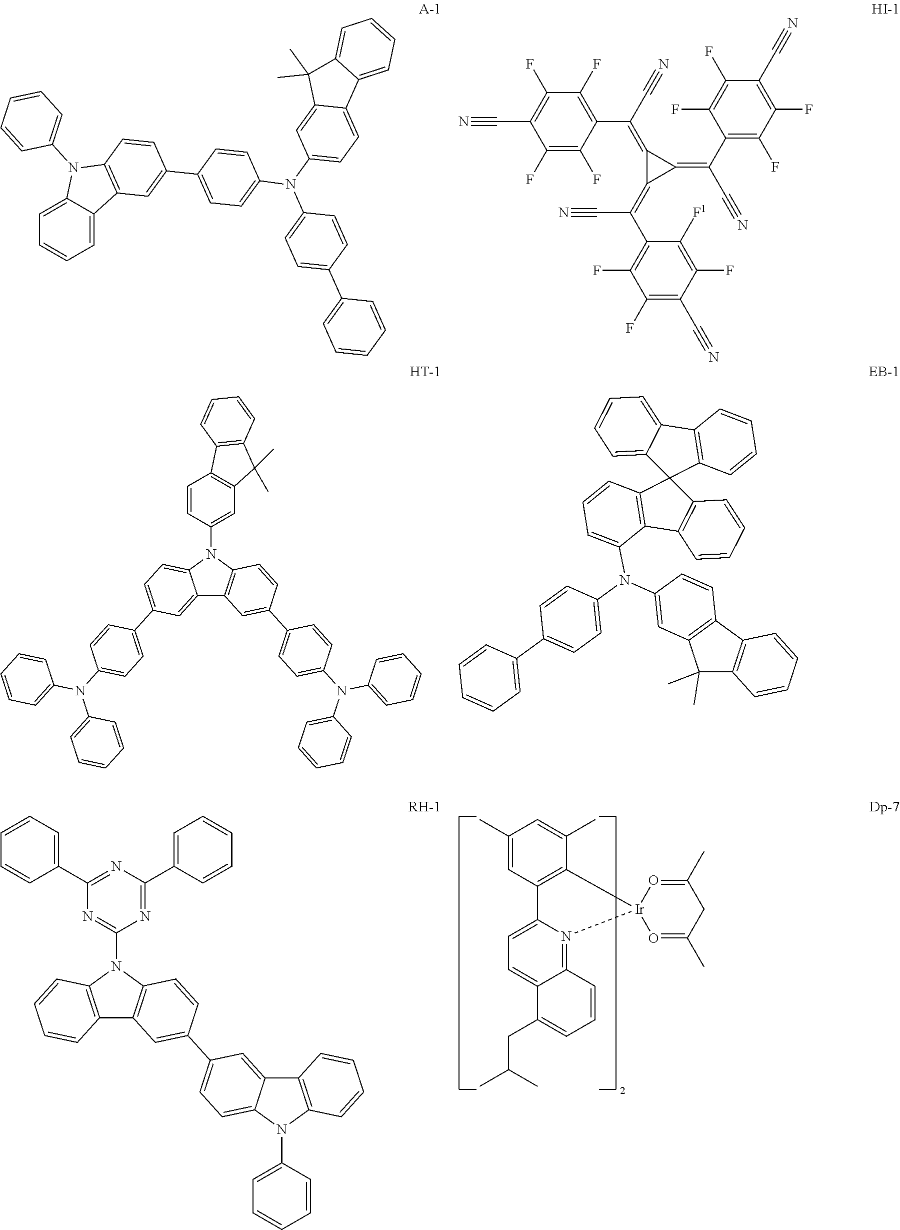

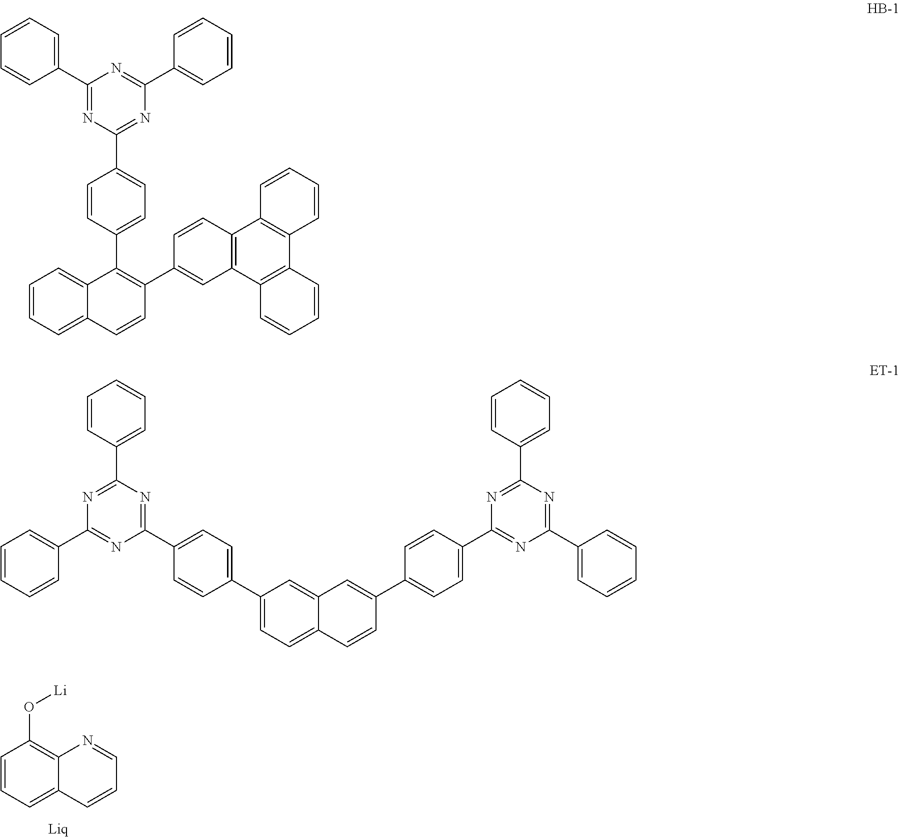

In the above process, the deposition rate of the organic material was maintained at 0.4 to 0.7 Å/sec, the deposition rate of lithium fluoride of the cathode was maintained at 0.3 Å/sec, and the deposition rate of aluminum was maintained at 2 Å/sec. In addition, the degree of vacuum during the deposition was maintained at 2×10⁻⁷ to 5×10⁻⁶ torr, thereby manufacturing an organic light emitting device.

### Examples 1 to 17

Organic light emitting devices were manufactured in the same manner as in Comparative Example A, except that the compound shown in Table 1 was used instead of the Compound RH-1 as a host in the organic light emitting device of Comparative Example A.

### Comparative Examples 1 to 7

Organic light emitting devices were manufactured in the same manner as in Comparative Example A, except that the compound shown in Table 1 was used instead of the Compound RH-1 as a host in the organic light emitting device of Comparative Example A. The structures of Compounds B-8 to B-14 of Table 1 are as follows.

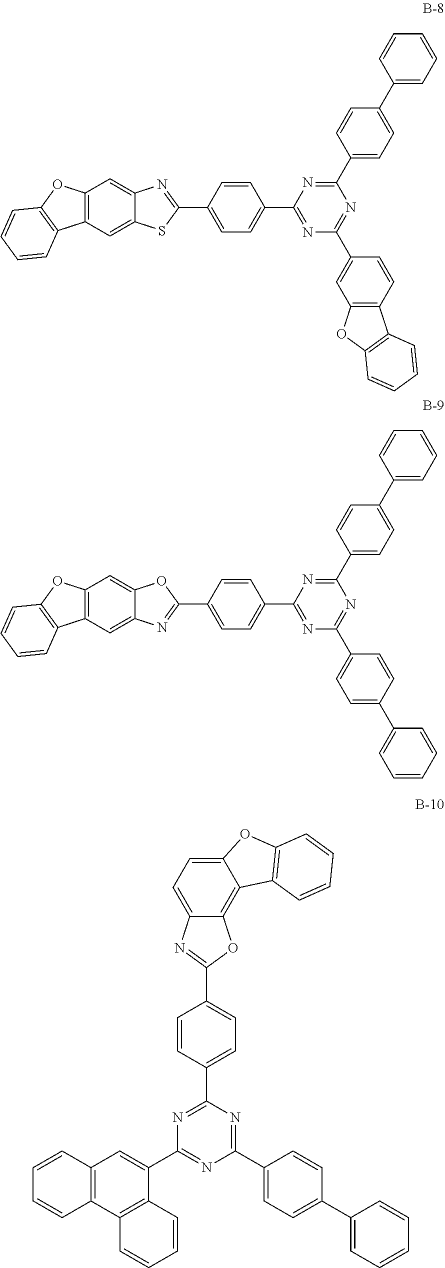

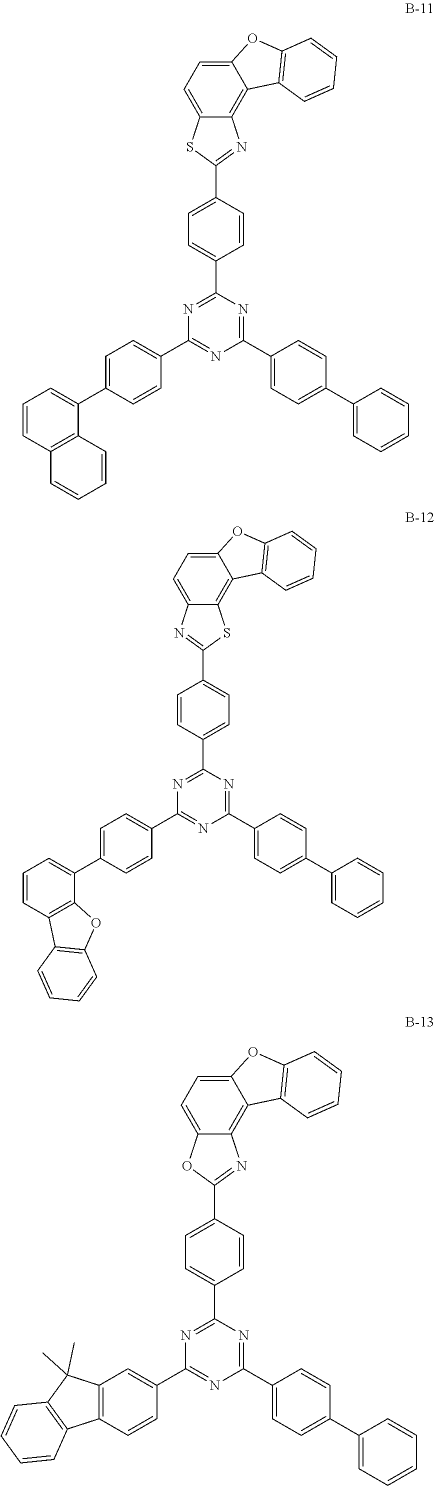 [SMILES: c1ccc(-c2ccc(-c3nc(-c4ccc(-c5nc6c(ccc7oc8ccccc8c76)s5)cc4)nc(-c4ccc(-c5cccc6ccccc56)cc4)n3)cc2)cc1 ; c1ccc(-c2ccc(-c3nc(-c4ccc(-c5nc6ccc7oc8ccccc8c7c6s5)cc4)nc(-c4ccc(-c5cccc6c5oc5ccccc56)cc4)n3)cc2)cc1 ; CC1(C)c2ccccc2-c2ccc(-c3nc(-c4ccc(-c5ccccc5)cc4)nc(-c4ccc(-c5nc6c(ccc7oc8ccccc8c76)o5)cc4)n3)cc21]

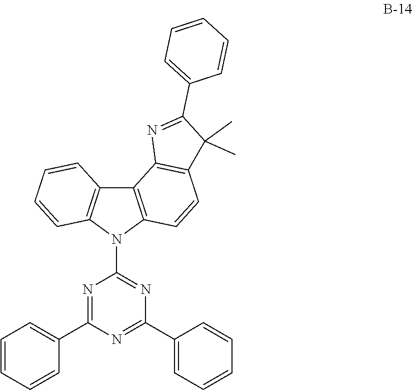 [SMILES: CC1(C)C(c2ccccc2)=Nc2c1ccc1c2c2ccccc2n1-c1nc(-c2ccccc2)nc(-c2ccccc2)n1]

### Examples 18 to 47

Organic light emitting devices were manufactured in the same manner as in Comparative Example A, except that the compound shown in Table 2 was used instead of the Compound EB-1 as an electron blocking layer material in the organic light emitting device of Comparative Example A.

### Comparative Examples 8 to 14

Organic light emitting devices were manufactured in the same manner as in Comparative Example A, except that the compound shown in Table 2 was used instead of the Compound EB-1 as an electron blocking layer material in the organic light emitting device of Comparative Example A. The structures of Compounds B-1 to B-7 of Table 2 are as follows.

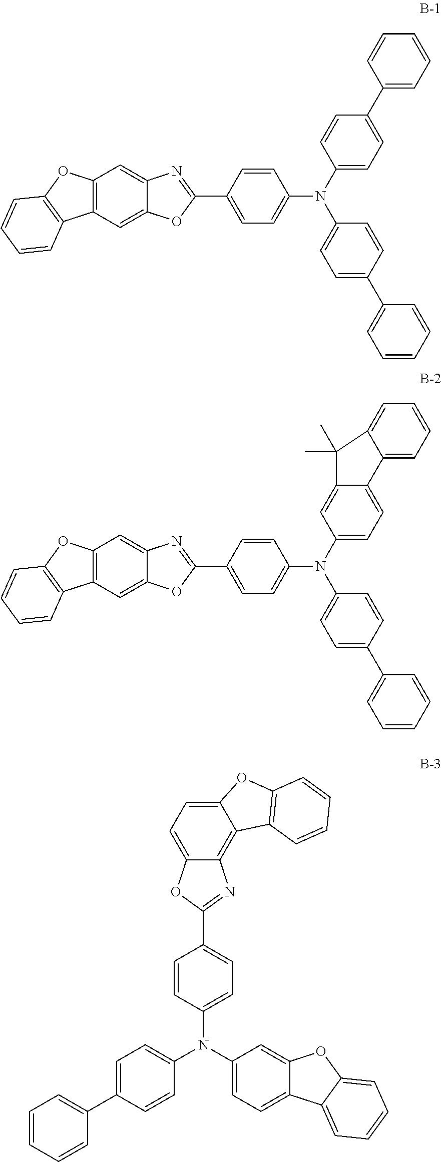 [SMILES: c1ccc(-c2ccc(N(c3ccc(-c4ccccc4)cc3)c3ccc(-c4nc5cc6oc7ccccc7c6cc5o4)cc3)cc2)cc1 ; CC1(C)c2ccccc2-c2ccc(N(c3ccc(-c4ccccc4)cc3)c3ccc(-c4nc5cc6oc7ccccc7c6cc5o4)cc3)cc21 ; c1ccc(-c2ccc(N(c3ccc(-c4nc5c(ccc6oc7ccccc7c65)o4)cc3)c3ccc4c(c3)oc3ccccc34)cc2)cc1]

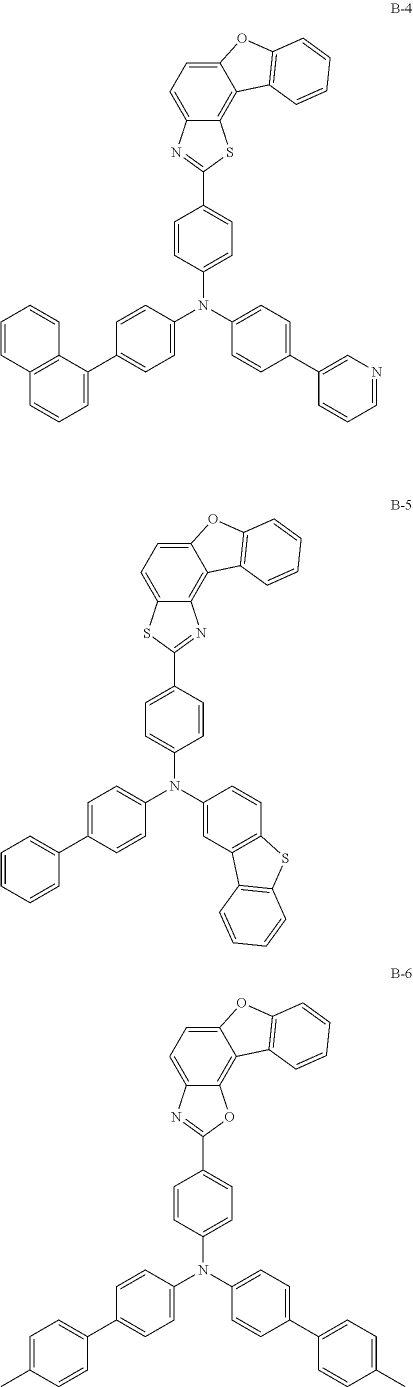

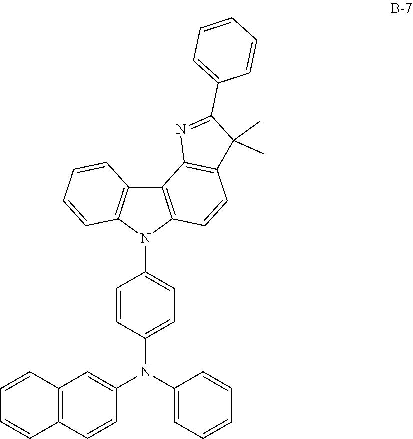 [SMILES: CC1(C)C(c2ccccc2)=Nc2c1ccc1c2c2ccccc2n1-c1ccc(N(c2ccccc2)c2ccc3ccccc3c2)cc1]

### Examples 48 to 115

Organic light emitting devices were manufactured in the same manner as in Comparative Example A, except that the compounds of the first host and the second host described in Table 3 were used in a weight ratio of 1:1 instead of the Compound RH-1 as a host in the organic light emitting device of Comparative Example A.

### EXPERIMENTAL EXAMPLES

The driving voltage, and efficiency (based on 15 mA/cm²) were measured by applying a current to the organic light emitting devices prepared in the above Examples 1 to 115, Comparative Example A and Comparative Examples 1 to 14, and the results are shown in Table 1 to Table 3. The lifespan T95 means the time taken until the initial luminance (7,000 nit) decreases to 95%.

TABLE 1

| | | Driving | | | Emis- |
|---|---|---|---|---|---|
| | | voltage | Efficiency | Lifespan | sion |
| Category | Host | (V) | (cd/A) | T95(hr) | color |
| Comparative | Compound RH-1 | 3.91 | 16.54 | 113 | Red |
| Example A | | | | | |
| Example 1 | Compound 1-1 | 3.63 | 19.35 | 178 | Red |
| Example 2 | Compound 1-2 | 3.65 | 19.89 | 187 | Red |
| Example 3 | Compound 1-3 | 3.61 | 20.14 | 196 | Red |
| Example 4 | Compound 1-4 | 3.54 | 20.86 | 217 | Red |
| Example 5 | Compound 1-5 | 3.59 | 20.35 | 203 | Red |
| Example 6 | Compound 1-6 | 3.63 | 19.67 | 186 | Red |
| Example 7 | Compound 1-7 | 3.72 | 19.03 | 173 | Red |
| Example 8 | Compound 1-8 | 3.69 | 20.27 | 193 | Red |
| Example 9 | Compound 1-9 | 3.73 | 19.38 | 182 | Red |
| Example 10 | Compound 1-10 | 3.76 | 18.86 | 186 | Red |
| Example 11 | Compound 1-11 | 3.81 | 18.51 | 179 | Red |
| Example 12 | Compound 1-12 | 3.83 | 18.30 | 167 | Red |
| Example 13 | Compound 1-13 | 3.72 | 19.34 | 193 | Red |
| Example 14 | Compound 1-14 | 3.75 | 19.02 | 190 | Red |
| Example 15 | Compound 1-15 | 3.80 | 18.45 | 172 | Red |
| Example 16 | Compound 1-16 | 3.84 | 18.26 | 169 | Red |
| Example 17 | Compound 1-17 | 3.76 | 18.93 | 184 | Red |
| Comparative | Compound B-8 | 4.05 | 16.92 | 98 | Red |
| Example 1 | | | | | |
| Comparative | Compound B-9 | 3.98 | 17.39 | 125 | Red |
| Example 2 | | | | | |
| Comparative | Compound B-10 | 3.95 | 17.64 | 133 | Red |
| Example 3 | | | | | |
| Comparative | Compound B-11 | 4.03 | 17.03 | 117 | Red |
| Example 4 | | | | | |
| Comparative | Compound B-12 | 3.97 | 16.85 | 102 | Red |
| Example 5 | | | | | |
| Comparative | Compound B-13 | 4.09 | 16.31 | 94 | Red |
| Example 6 | | | | | |
| Comparative | Compound B-14 | 4.11 | 12.76 | 76 | Red |
| Example 7 | | | | | |

TABLE 2

| | | Driving | | | Emis- |
|---|---|---|---|---|---|
| | Electron | voltage | Efficiency | Lifespan | sion |
| Category | blocking layer | (V) | (cd/A) | T95(hr) | color |
| Example 18 | Compound 2-1 | 3.71 | 18.72 | 159 | Red |
| Example 19 | Compound 2-2 | 3.78 | 18.75 | 167 | Red |
| Example 20 | Compound 2-3 | 3.75 | 18.22 | 172 | Red |
| Example 21 | Compound 2-4 | 3.82 | 19.19 | 161 | Red |
| Example 22 | Compound 2-5 | 3.79 | 18.64 | 164 | Red |
| Example 23 | Compound 2-6 | 3.73 | 18.36 | 166 | Red |
| Example 24 | Compound 2-7 | 3.71 | 18.67 | 179 | Red |
| Example 25 | Compound 2-8 | 3.84 | 18.76 | 185 | Red |
| Example 26 | Compound 2-9 | 3.67 | 18.96 | 165 | Red |
| Example 27 | Compound 2-10 | 3.69 | 19.37 | 186 | Red |
| Example 28 | Compound 2-11 | 3.77 | 19.82 | 183 | Red |
| Example 29 | Compound 2-12 | 3.67 | 20.51 | 178 | Red |
| Example 30 | Compound 2-13 | 3.71 | 19.12 | 184 | Red |
| Example 31 | Compound 2-14 | 3.78 | 20.61 | 171 | Red |
| Example 32 | Compound 2-15 | 3.72 | 19.26 | 169 | Red |
| Example 33 | Compound 2-16 | 3.84 | 21.63 | 183 | Red |
| Example 34 | Compound 2-17 | 3.75 | 21.97 | 180 | Red |
| Example 35 | Compound 2-18 | 3.78 | 20.55 | 174 | Red |
| Example 36 | Compound 2-19 | 3.76 | 19.49 | 186 | Red |
| Example 37 | Compound 2-20 | 3.67 | 20.45 | 187 | Red |
| Example 38 | Compound 2-21 | 3.80 | 20.10 | 180 | Red |
| Example 39 | Compound 2-22 | 3.81 | 19.44 | 175 | Red |
| Example 40 | Compound 2-23 | 3.78 | 21.76 | 164 | Red |
| Example 41 | Compound 2-24 | 3.74 | 18.84 | 193 | Red |
| Example 42 | Compound 2-25 | 3.81 | 20.37 | 179 | Red |
| Example 43 | Compound 2-26 | 3.71 | 18.69 | 183 | Red |
| Example 44 | Compound 2-27 | 3.72 | 19.46 | 176 | Red |
| Example 45 | Compound 2-28 | 3.83 | 20.97 | 168 | Red |
| Example 46 | Compound 2-29 | 3.82 | 18.93 | 179 | Red |
| Example 47 | Compound 2-30 | 3.80 | 20.08 | 175 | Red |
| Comparative | Compound B-1 | 3.96 | 16.36 | 104 | Red |
| Example 8 | | | | | |
| Comparative | Compound B-2 | 4.03 | 17.09 | 127 | Red |
| Example 9 | | | | | |
| Comparative | Compound B-3 | 3.92 | 17.12 | 138 | Red |
| Example 10 | | | | | |
| Comparative | Compound B-4 | 3.96 | 16.23 | 106 | Red |
| Example 11 | | | | | |
| Comparative | Compound B-5 | 3.94 | 16.18 | 93 | Red |
| Example 12 | | | | | |
| Comparative | Compound B-6 | 4.13 | 15.60 | 71 | Red |
| Example 13 | | | | | |
| Comparative | Compound B-7 | 4.27 | 12.21 | 84 | Red |
| Example 14 | | | | | |

TABLE 3

| | | | Driving | | Lifespan | |
|---|---|---|---|---|---|---|
| | | | voltage | Efficiency | T95 | Emission |
| Category | First host | Second host | (V) | (cd/A) | (hr) | color |
| Example 48 | Compound 1-1 | Compound 2-1 | 3.49 | 21.52 | 218 | Red |
| Example 49 | Compound 1-1 | Compound 2-4 | 3.50 | 21.87 | 223 | Red |
| Example 50 | Compound 1-1 | Compound 2-10 | 3.41 | 20.19 | 216 | Red |
| Example 51 | Compound 1-1 | Compound 2-15 | 3.57 | 21.10 | 221 | Red |
| Example 52 | Compound 1-1 | Compound 2-22 | 3.45 | 19.82 | 207 | Red |
| Example 53 | Compound 1-1 | Compound 2-26 | 3.49 | 19.84 | 208 | Red |
| Example 56 | Compound 1-3 | Compound 2-2 | 3.65 | 23.65 | 229 | Red |
| Example 57 | Compound 1-3 | Compound 2-5 | 3.63 | 23.18 | 220 | Red |
| Example 58 | Compound 1-3 | Compound 2-11 | 3.65 | 21.54 | 230 | Red |
| Example 59 | Compound 1-3 | Compound 2-16 | 3.48 | 23.31 | 233 | Red |
| Example 60 | Compound 1-3 | Compound 2-23 | 3.62 | 22.16 | 212 | Red |
| Example 61 | Compound 1-3 | Compound 2-27 | 3.46 | 23.65 | 212 | Red |
| Example 62 | Compound 1-4 | Compound 2-3 | 3.49 | 23.86 | 237 | Red |
| Example 63 | Compound 1-4 | Compound 2-6 | 3.47 | 23.17 | 217 | Red |
| Example 64 | Compound 1-4 | Compound 2-12 | 3.44 | 23.77 | 232 | Red |
| Example 65 | Compound 1-4 | Compound 2-17 | 3.51 | 22.92 | 232 | Red |
| Example 66 | Compound 1-4 | Compound 2-24 | 3.56 | 24.00 | 229 | Red |
| Example 67 | Compound 1-4 | Compound 2-28 | 3.49 | 22.88 | 232 | Red |
| Example 68 | Compound 1-5 | Compound 2-1 | 3.42 | 19.68 | 216 | Red |
| Example 69 | Compound 1-5 | Compound 2-4 | 3.40 | 20.99 | 221 | Red |
| Example 70 | Compound 1-5 | Compound 2-13 | 3.56 | 19.58 | 214 | Red |
| Example 71 | Compound 1-5 | Compound 2-18 | 3.40 | 20.84 | 206 | Red |
| Example 72 | Compound 1-5 | Compound 2-25 | 3.50 | 21.32 | 218 | Red |
| Example 73 | Compound 1-5 | Compound 2-29 | 3.40 | 19.56 | 222 | Red |
| Example 74 | Compound 1-8 | Compound 2-1 | 3.51 | 23.01 | 215 | Red |
| Example 75 | Compound 1-8 | Compound 2-4 | 3.62 | 23.29 | 221 | Red |
| Example 76 | Compound 1-8 | Compound 2-10 | 3.64 | 23.17 | 216 | Red |
| Example 77 | Compound 1-8 | Compound 2-15 | 3.63 | 21.39 | 233 | Red |
| Example 78 | Compound 1-8 | Compound 2-22 | 3.63 | 22.22 | 238 | Red |
| Example 79 | Compound 1-8 | Compound 2-26 | 3.61 | 22.77 | 234 | Red |
| Example 80 | Compound 1-9 | Compound 2-2 | 3.45 | 22.43 | 210 | Red |
| Example 81 | Compound 1-9 | Compound 2-5 | 3.63 | 22.06 | 218 | Red |
| Example 82 | Compound 1-9 | Compound 2-11 | 3.60 | 21.97 | 214 | Red |
| Example 83 | Compound 1-9 | Compound 2-16 | 3.58 | 19.40 | 214 | Red |
| Example 84 | Compound 1-9 | Compound 2-23 | 3.59 | 21.49 | 209 | Red |
| Example 85 | Compound 1-9 | Compound 2-27 | 3.58 | 21.86 | 224 | Red |
| Example 86 | Compound 1-10 | Compound 2-3 | 3.45 | 23.54 | 224 | Red |
| Example 87 | Compound 1-10 | Compound 2-6 | 3.46 | 22.53 | 214 | Red |
| Example 88 | Compound 1-10 | Compound 2-12 | 3.50 | 23.21 | 217 | Red |
| Example 89 | Compound 1-10 | Compound 2-17 | 3.48 | 23.16 | 226 | Red |
| Example 90 | Compound 1-10 | Compound 2-24 | 3.65 | 21.57 | 206 | Red |
| Example 91 | Compound 1-10 | Compound 2-28 | 3.59 | 22.68 | 206 | Red |
| Example 92 | Compound 1-13 | Compound 2-1 | 3.46 | 21.32 | 223 | Red |
| Example 93 | Compound 1-13 | Compound 2-4 | 3.60 | 20.86 | 212 | Red |
| Example 94 | Compound 1-13 | Compound 2-13 | 3.41 | 20.17 | 211 | Red |
| Example 95 | Compound 1-13 | Compound 2-18 | 3.42 | 21.64 | 221 | Red |
| Example 96 | Compound 1-13 | Compound 2-25 | 3.52 | 20.87 | 209 | Red |
| Example 97 | Compound 1-13 | Compound 2-29 | 3.50 | 21.27 | 216 | Red |
| Example 98 | Compound 1-14 | Compound 2-1 | 3.55 | 24.25 | 217 | Red |
| Example 99 | Compound 1-14 | Compound 2-4 | 3.36 | 24.03 | 219 | Red |
| Example 100 | Compound 1-14 | Compound 2-10 | 3.57 | 23.66 | 240 | Red |
| Example 101 | Compound 1-14 | Compound 2-15 | 3.35 | 23.52 | 240 | Red |
| Example 102 | Compound 1-14 | Compound 2-22 | 3.41 | 23.56 | 223 | Red |
| Example 103 | Compound 1-14 | Compound 2-26 | 3.56 | 23.66 | 219 | Red |
| Example 104 | Compound 1-15 | Compound 2-2 | 3.49 | 21.05 | 226 | Red |
| Example 105 | Compound 1-15 | Compound 2-5 | 3.60 | 21.68 | 211 | Red |
| Example 106 | Compound 1-15 | Compound 2-11 | 3.48 | 21.49 | 210 | Red |
| Example 107 | Compound 1-15 | Compound 2-16 | 3.60 | 22.04 | 215 | Red |
| Example 108 | Compound 1-15 | Compound 2-23 | 3.56 | 22.26 | 235 | Red |
| Example 109 | Compound 1-15 | Compound 2-27 | 3.65 | 22.87 | 228 | Red |
| Example 110 | Compound 1-16 | Compound 2-3 | 3.41 | 19.24 | 215 | Red |
| Example 111 | Compound 1-16 | Compound 2-6 | 3.45 | 20.36 | 215 | Red |
| Example 112 | Compound 1-16 | Compound 2-12 | 3.47 | 19.91 | 215 | Red |
| Example 113 | Compound 1-16 | Compound 2-17 | 3.44 | 21.52 | 224 | Red |
| Example 114 | Compound 1-16 | Compound 2-24 | 3.58 | 21.40 | 216 | Red |
| Example 115 | Compound 1-16 | Compound 2-28 | 3.42 | 21.17 | 220 | Red |

When a current was applied to the organic light emitting devices manufactured in Examples 1 to 115 and Comparative Examples 1 to 14, the results shown in Tables 1 to 3 were obtained.

It was confirmed that when the Compounds 1-1 to 1-17 of the present disclosure were used as a red host, the driving voltage decreased and the efficiency and lifespan increased compared to the case of using the compound of Comparative Example as shown in Table 1. Even when the Compounds 2-1 to 2-30 of the present disclosure were used as the electron blocking layer, the driving voltage decreased and the efficiency and lifespan increased compared to the case of using the compound of Comparative Example as shown in Table 2.

Additionally, it was confirmed in Table 3 that when one of Compounds 1-1 to 1-17 was selected as a first host and one of Compounds 2-1 to 2-30 was used as a second host, and they were used as a red host by co-deposition, the driving voltage decreased and the efficiency and lifespan increased compared to the case of using a single material host.

That is, it was confirmed from the results of Tables 1 to 3 that when the compound of one embodiment was used as a host of the red light emitting layer or as an electron blocking layer in the red device, the driving voltage, luminous efficiency and lifespan characteristics of the organic light emitting device could be improved.

### DESCRIPTION OF SYMBOLS

| | |
|---|---|
| 1: Substrate | 2: Anode |
| 3: Light emitting layer | 4: Cathode |
| 5: Hole injection layer | 6: Hole transport layer |
| 7: Electron blocking layer | 8: Hole blocking layer |
| 9: Electron injection and transport layer | |

1. A compound of Chemical Formula 1: 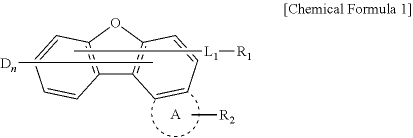
wherein in the Chemical Formula 1;
A is a thiazole ring or an oxazole ring fused with an adjacent ring;
L₁ is a single bond, substituted or unsubstituted C₆₋₆₀ arylene, or substituted or unsubstituted C₂₋₆₀ heteroarylene containing at least one heteroatom selected from the group consisting of N, O and S;
R₁ is 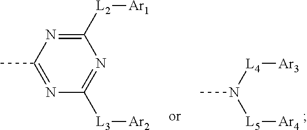
Ar₁ to Ar₄ are each independently substituted or unsubstituted C₆₋₆₀ aryl or substituted or unsubstituted C₂₋₆₀ heteroaryl containing at least one selected from the group consisting of N, O and S;
L₂ to L₅ are each independently a single bond, substituted or unsubstituted C₆₋₆₀ arylene, or substituted or unsubstituted C₂₋₆₀ heteroarylene containing at least one heteroatom selected from the group consisting of N, O and S;
R₂ is a substituted or unsubstituted C₆₋₆₀ aryl or substituted or unsubstituted C₂₋₆₀ heteroaryl containing at least one heteroatom selected from the group consisting of N, O and S;
D is deuterium; and
n is an integer of 0 to 5.

2. The compound of claim 1, wherein the Chemical Formula 1 is any one of the following Chemical Formulae 1-1 to 1-4: 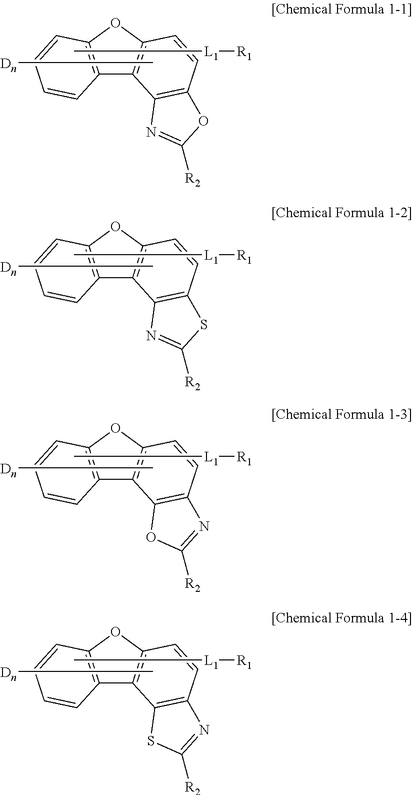
wherein in the Chemical Formulae 1-1 to 1-4,
R₁, R₂, Li, D and n are as defined in claim 1.

3. The compound of claim 1, wherein Li is a single bond, phenylene, biphenyldiyl, or naphthalenediyl.

4. The compound of claim 1, wherein Ar₁ and Ar₂ are each independently phenyl, biphenylyl, naphthyl, phenanthrenyl, dibenzofuranyl, or dibenzothiophenyl.

5. The compound of claim 1, wherein Ar₁ and Ar₄ are each independently phenyl, biphenylyl, terphenylyl, naphthyl, phenanthrenyl, dibenzofuranyl, dibenzothiophenyl, phenyl carbazolyl, or phenyl naphthyl.

6. The compound of claim 1, wherein L₂ and L₃ are each independently a single bond, phenylene, or naphthalenediyl.

7. The compound of claim 1, wherein L₄ and L₅ are each independently a single bond, phenylene, biphenyldiyl, naphthalenediyl, or carbazolediyl.

8. The compound of claim 1, wherein at least one of Ar₁ and Ar₂ is a substituted or unsubstituted C₆₋₆₀ aryl.

9. The compound of claim 1, wherein at least one of Ar₃ and Ar₄ is a substituted or unsubstituted C₆₋₆₀ aryl.

10. The compound of claim 1, wherein R₂ is phenyl, biphenylyl, naphthyl, dibenzofuranyl, or dibenzothiophenyl.

11. The compound of claim 1, wherein the compound of Chemical Formula 1 is any one compound selected from the group consisting of the following compounds:

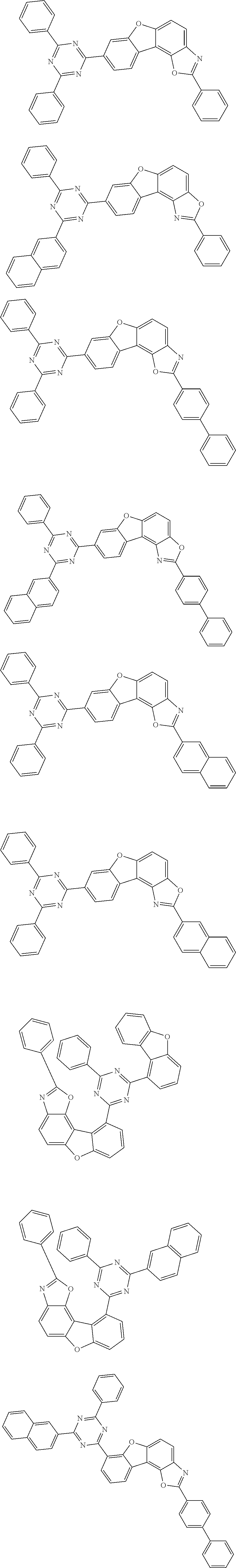

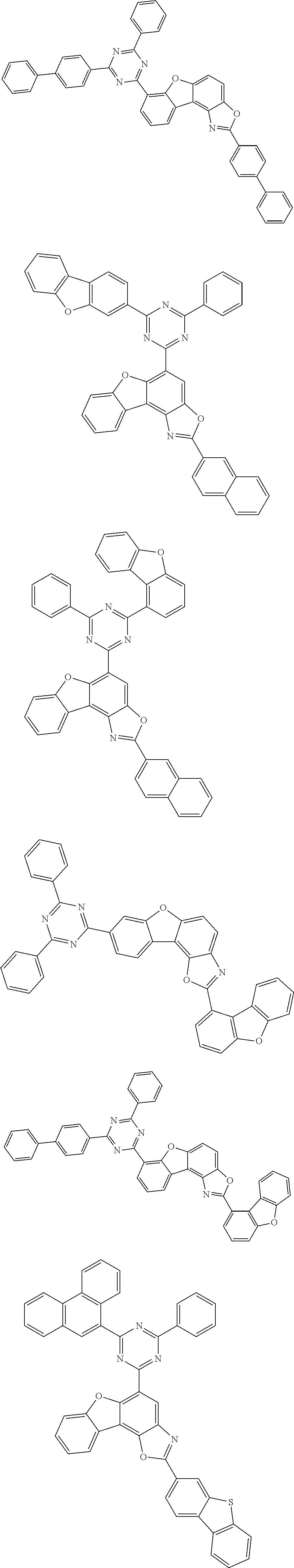

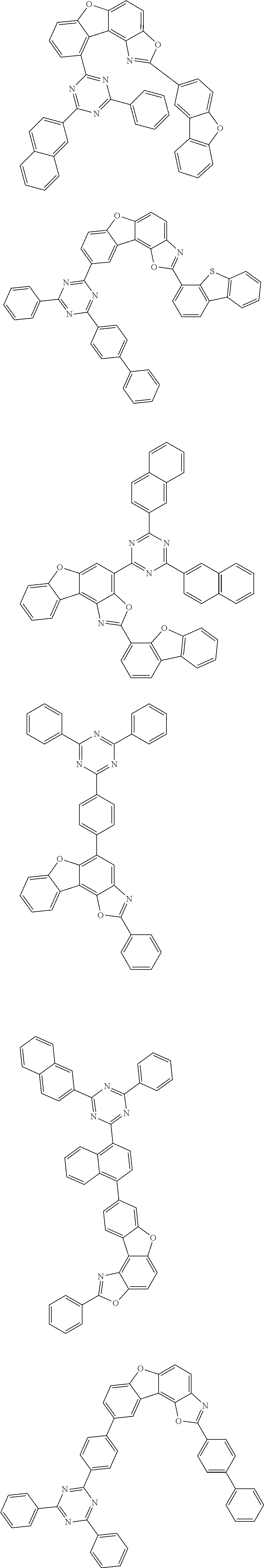

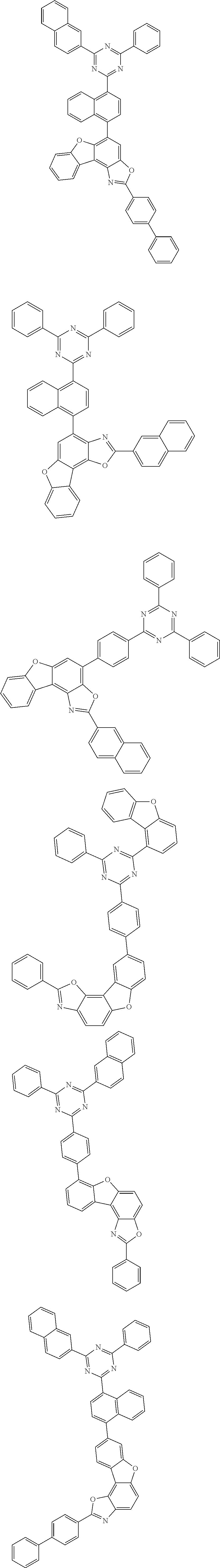

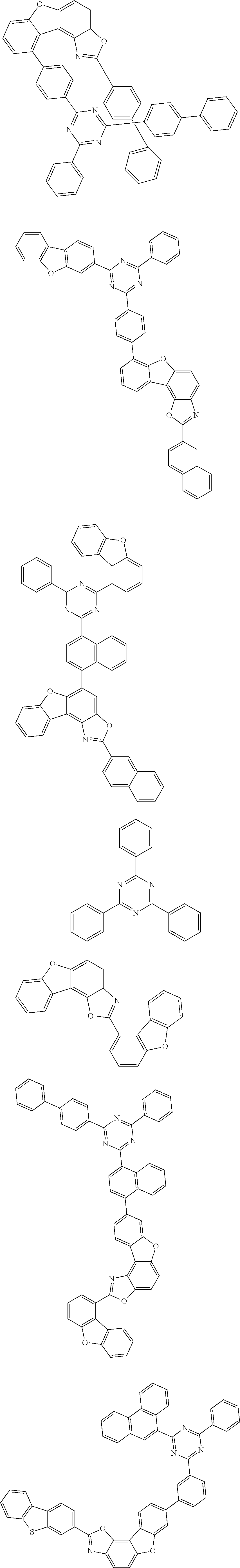

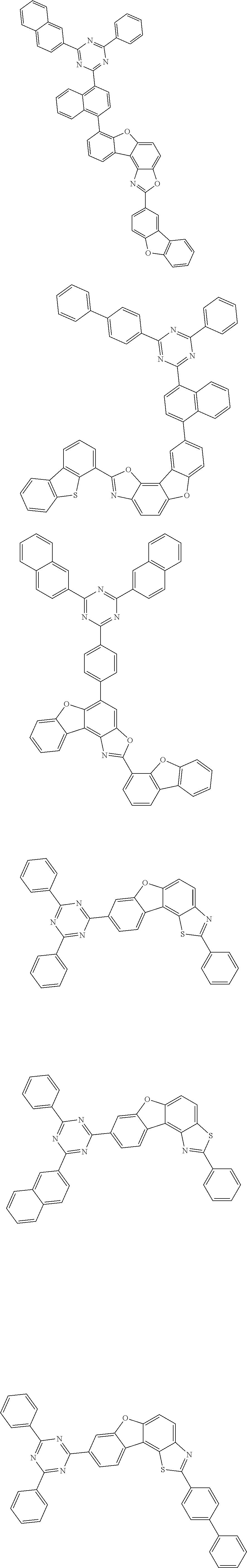

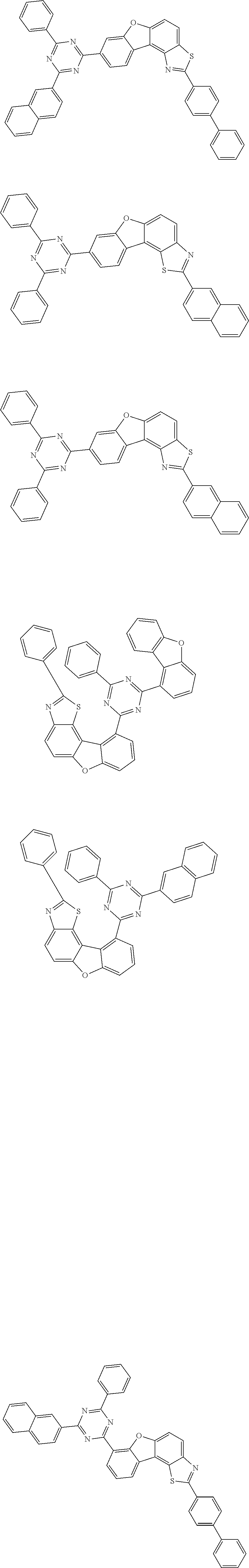 [SMILES: c1ccc(-c2ccc(-c3nc4c(ccc5oc6cc(-c7nc(-c8ccccc8)nc(-c8ccc9ccccc9c8)n7)ccc6c54)s3)cc2)cc1 ; c1ccc(-c2nc(-c3ccccc3)nc(-c3ccc4c(c3)oc3ccc5nc(-c6ccc7ccccc7c6)sc5c34)n2)cc1 ; c1ccc(-c2nc(-c3ccccc3)nc(-c3ccc4c(c3)oc3ccc5sc(-c6ccc7ccccc7c6)nc5c34)n2)cc1 ; c1ccc(-c2nc(-c3cccc4oc5ccccc5c34)nc(-c3cccc4oc5ccc6nc(-c7ccccc7)sc6c5c34)n2)cc1 ; c1ccc(-c2nc(-c3ccc4ccccc4c3)nc(-c3cccc4oc5ccc6nc(-c7ccccc7)sc6c5c34)n2)cc1 ; c1ccc(-c2ccc(-c3nc4ccc5oc6c(-c7nc(-c8ccccc8)nc(-c8ccc9ccccc9c8)n7)cccc6c5c4s3)cc2)cc1]

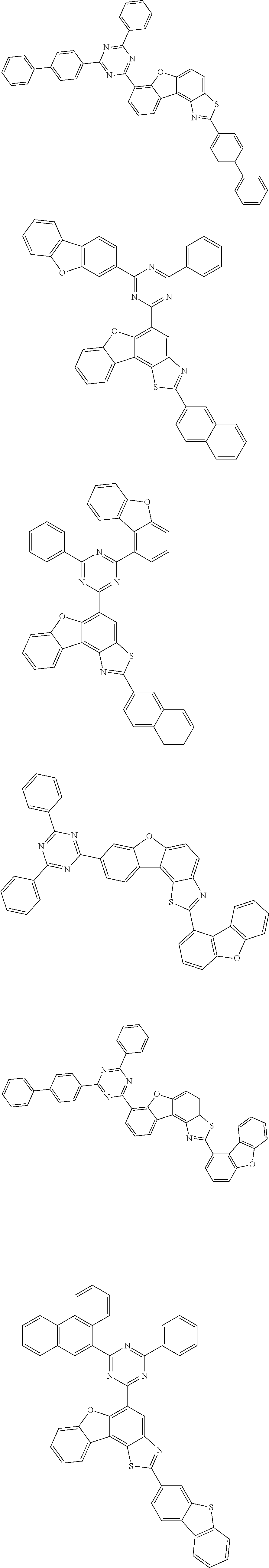

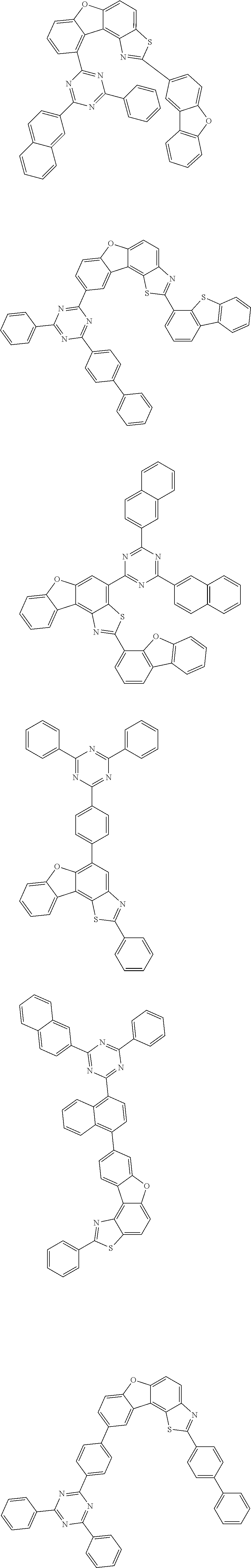

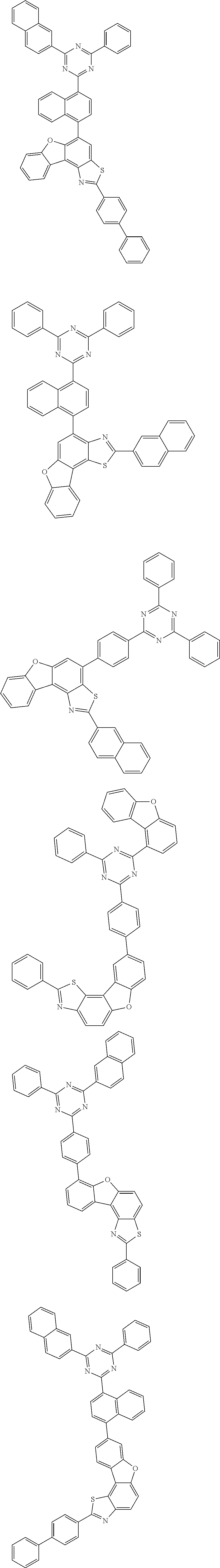

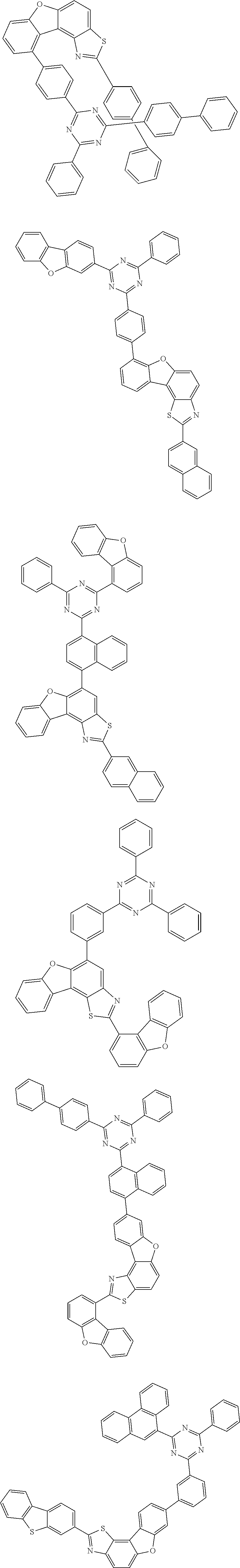

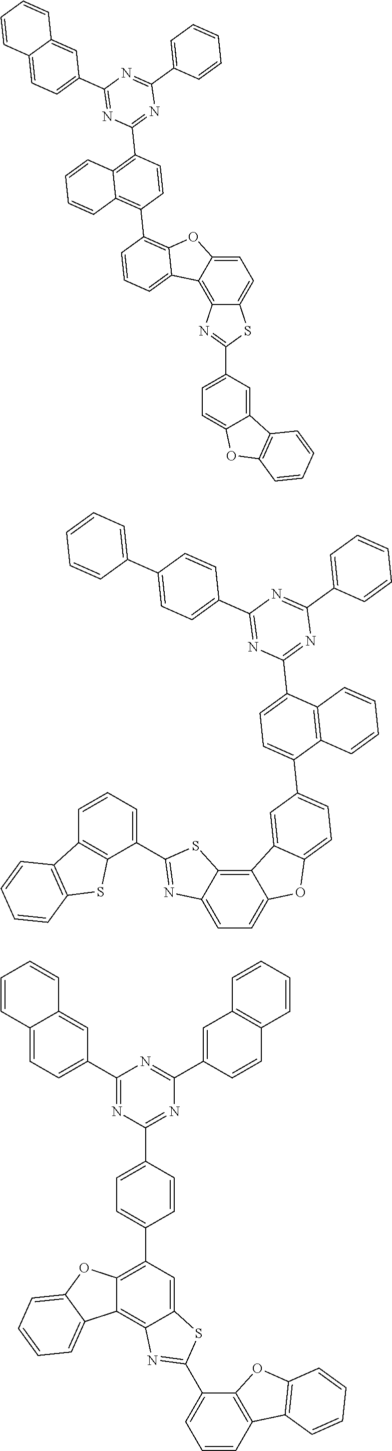

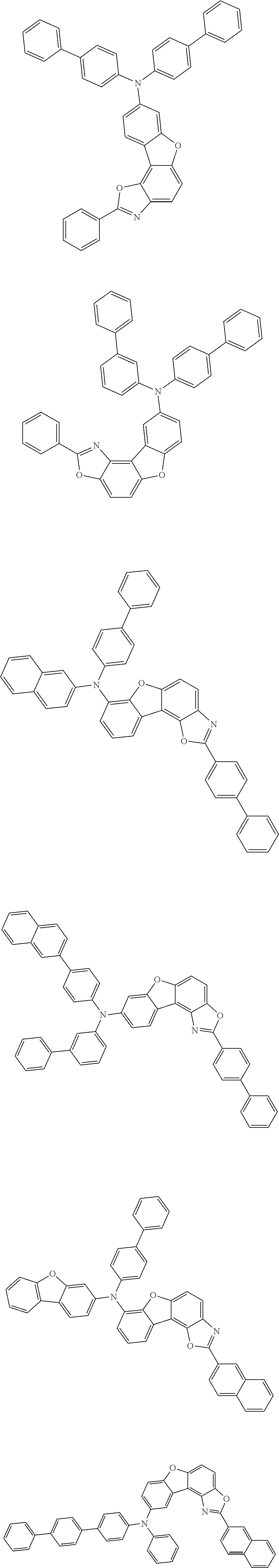

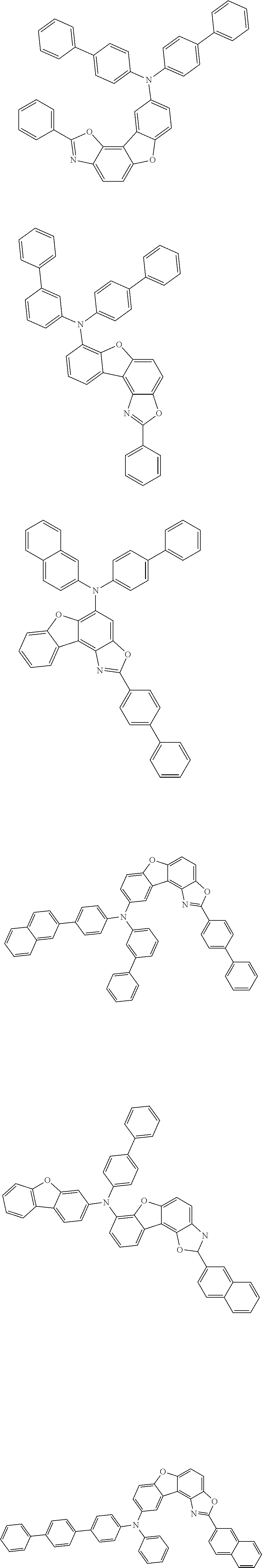

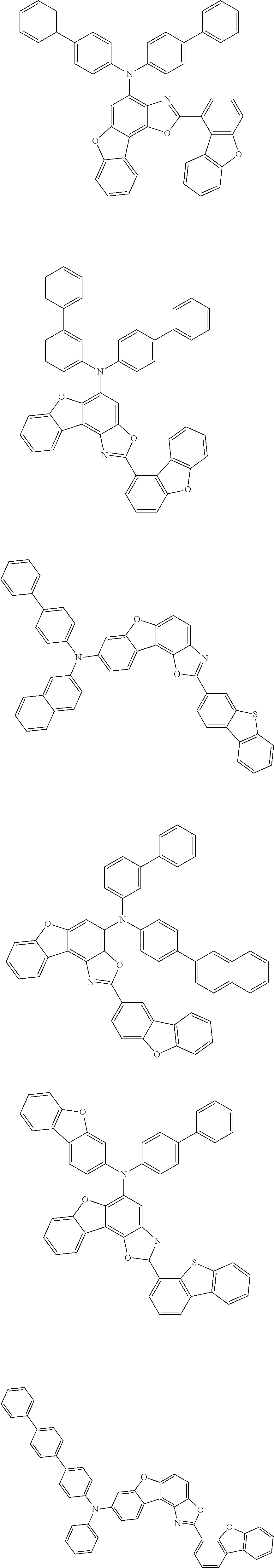

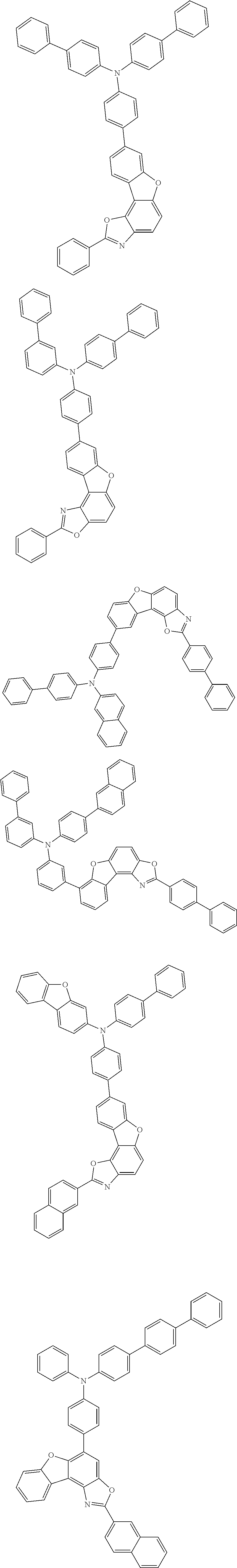

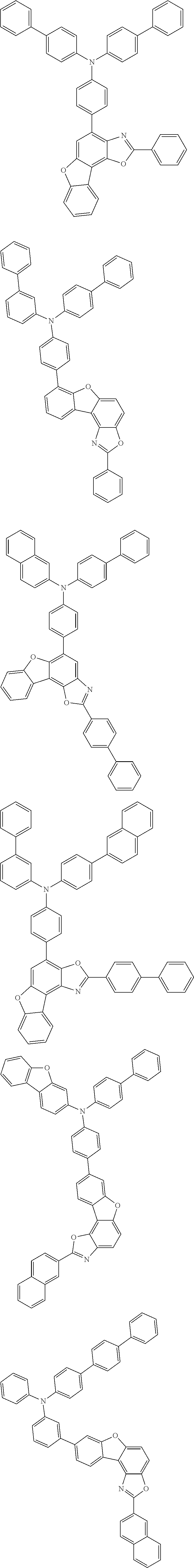

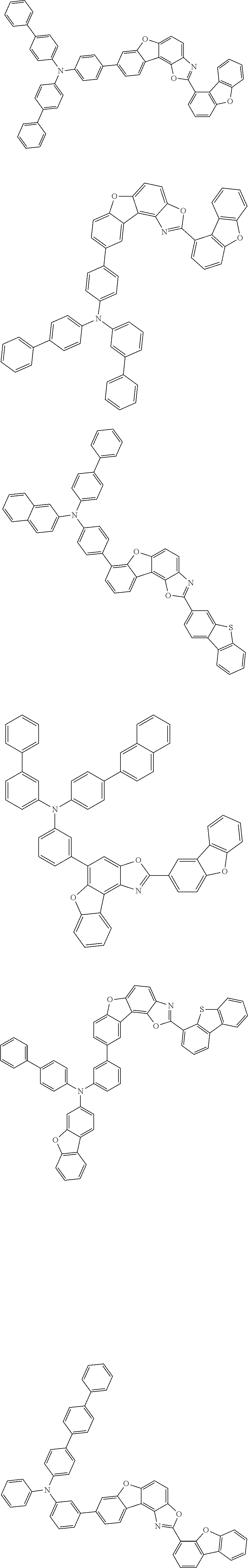

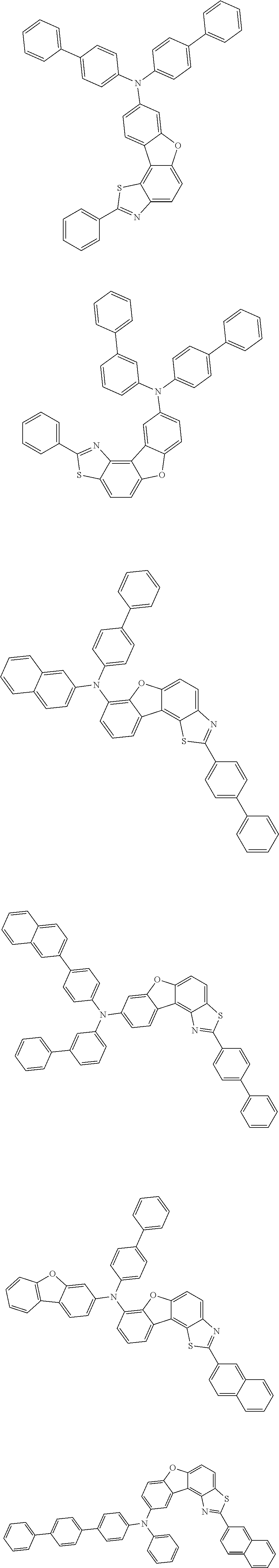

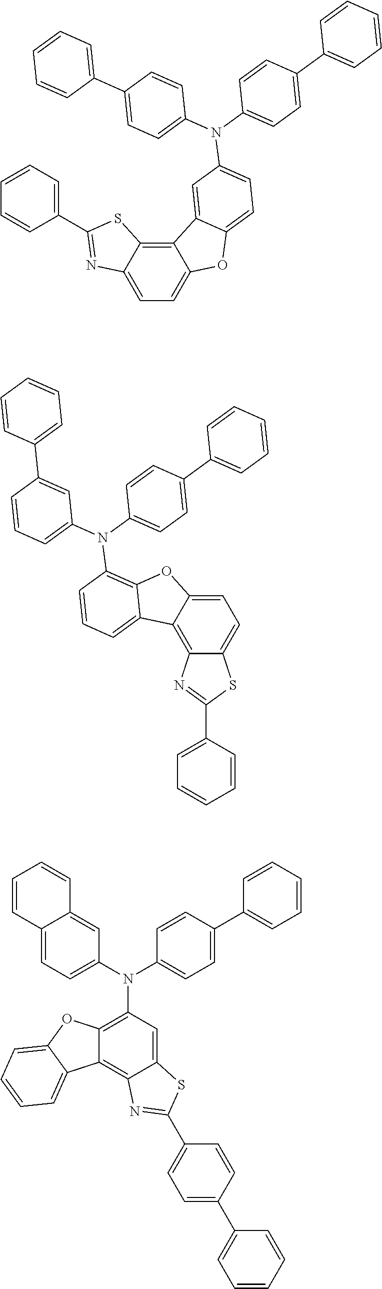 [SMILES: c1ccc(-c2ccc(N(c3ccc(-c4ccccc4)cc3)c3ccc4oc5ccc6nc(-c7ccccc7)sc6c5c4c3)cc2)cc1 ; c1ccc(-c2ccc(N(c3cccc(-c4ccccc4)c3)c3cccc4c3oc3ccc5sc(-c6ccccc6)nc5c34)cc2)cc1 ; c1ccc(-c2ccc(-c3nc4c(cc(N(c5ccc(-c6ccccc6)cc5)c5ccc6ccccc6c5)c5oc6ccccc6c54)s3)cc2)cc1]

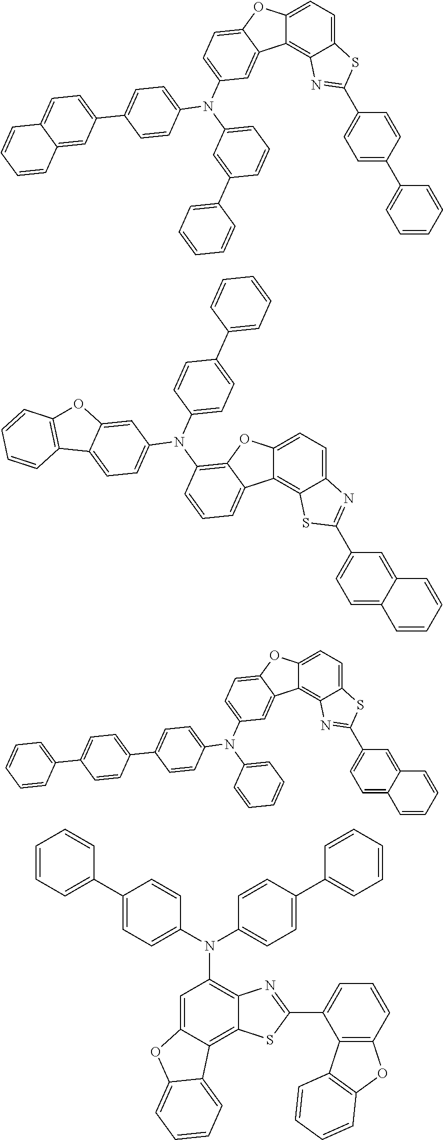

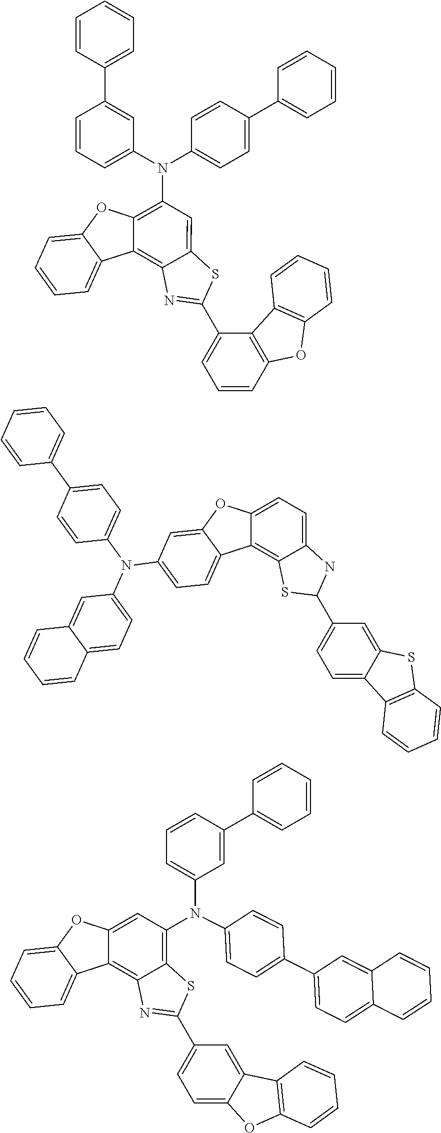

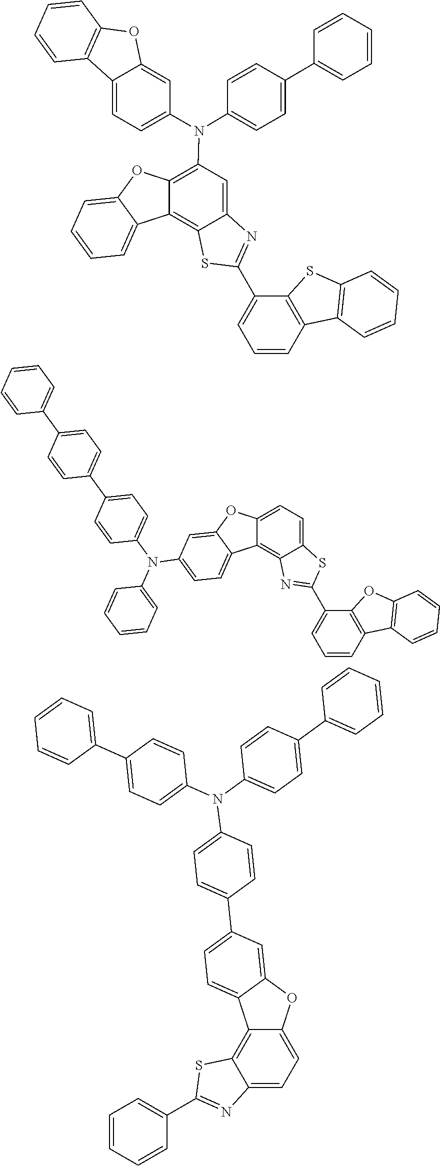

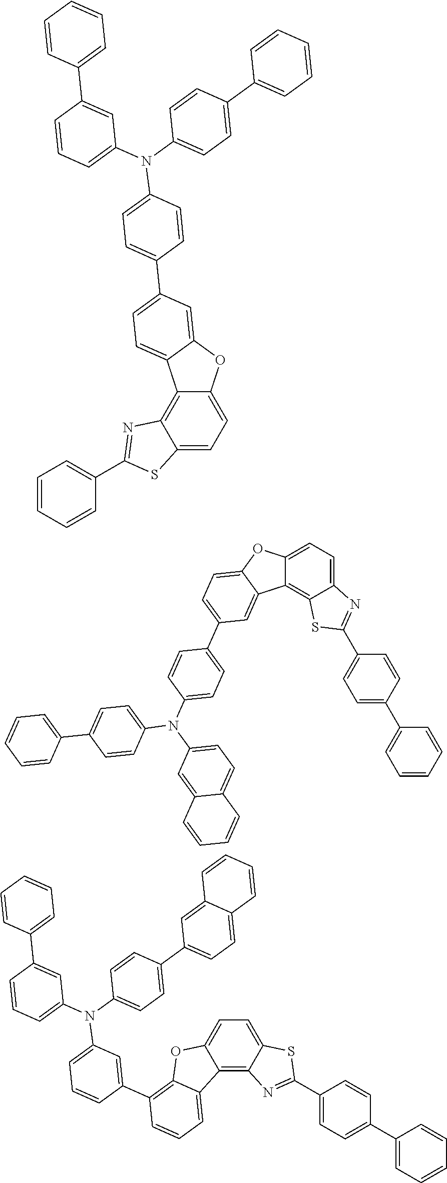

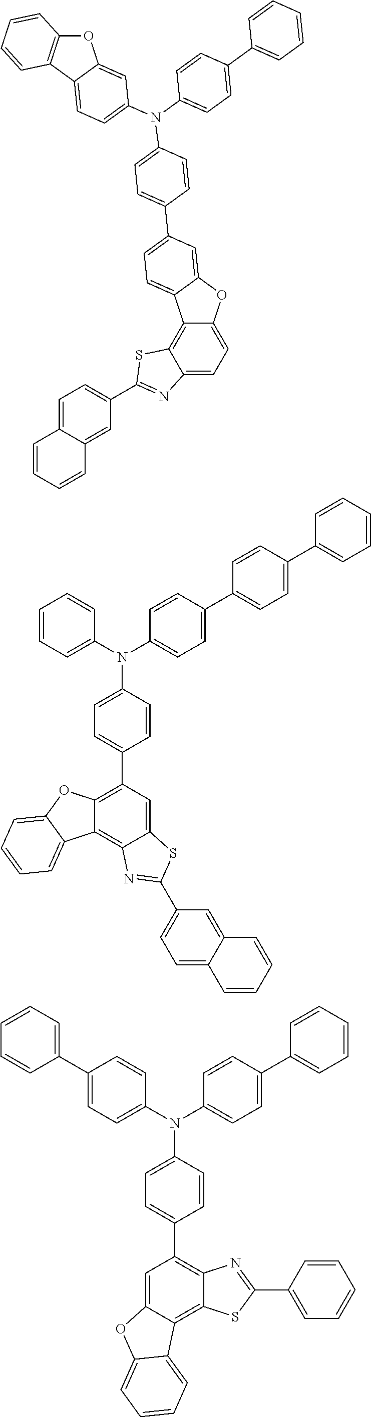 [SMILES: c1ccc(-c2ccc(N(c3ccc(-c4ccc5c(c4)oc4ccc6nc(-c7ccc8ccccc8c7)sc6c45)cc3)c3ccc4c(c3)oc3ccccc34)cc2)cc1 ; c1ccc(-c2ccc(-c3ccc(N(c4ccccc4)c4ccc(-c5cc6sc(-c7ccc8ccccc8c7)nc6c6c5oc5ccccc56)cc4)cc3)cc2)cc1 ; c1ccc(-c2ccc(N(c3ccc(-c4ccccc4)cc3)c3ccc(-c4cc5oc6ccccc6c5c5sc(-c6ccccc6)nc45)cc3)cc2)cc1]

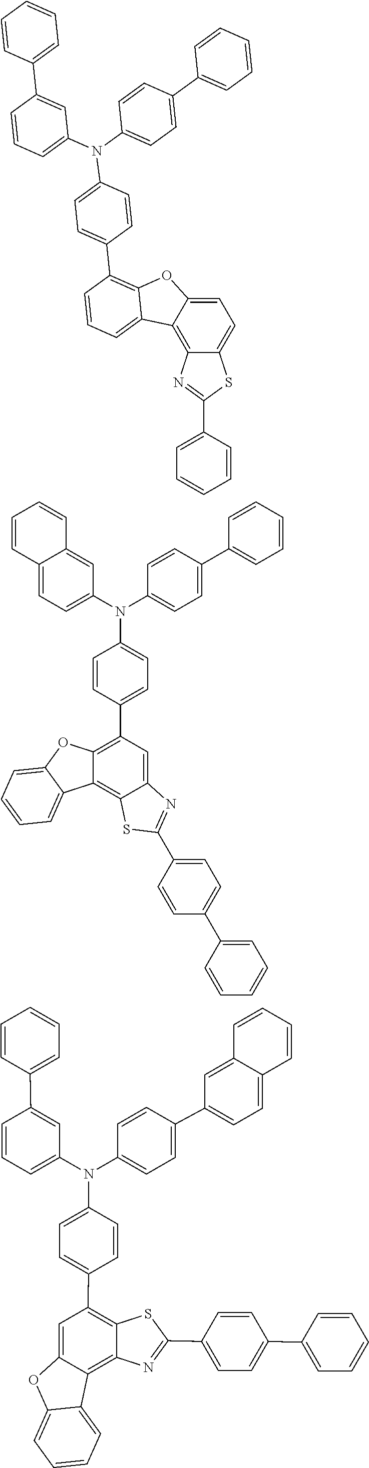

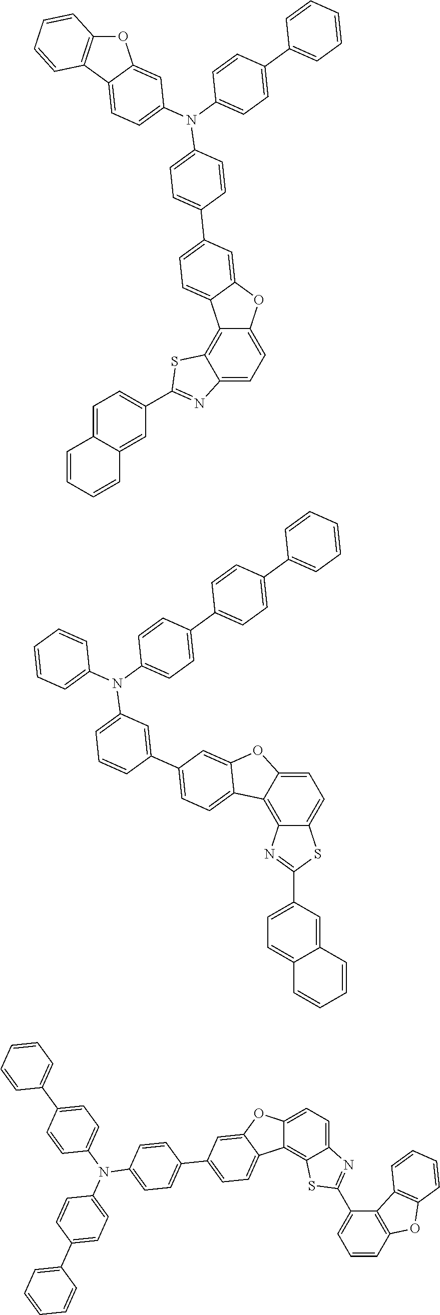 [SMILES: c1ccc(-c2ccc(N(c3ccc(-c4ccc5c(c4)oc4ccc6nc(-c7ccc8ccccc8c7)sc6c45)cc3)c3ccc4c(c3)oc3ccccc34)cc2)cc1 ; c1ccc(-c2ccc(-c3ccc(N(c4ccccc4)c4cccc(-c5ccc6c(c5)oc5ccc7sc(-c8ccc9ccccc9c8)nc7c56)c4)cc3)cc2)cc1 ; c1ccc(-c2ccc(N(c3ccc(-c4ccccc4)cc3)c3ccc(-c4ccc5c(c4)oc4ccc6nc(-c7cccc8oc9ccccc9c78)sc6c45)cc3)cc2)cc1]

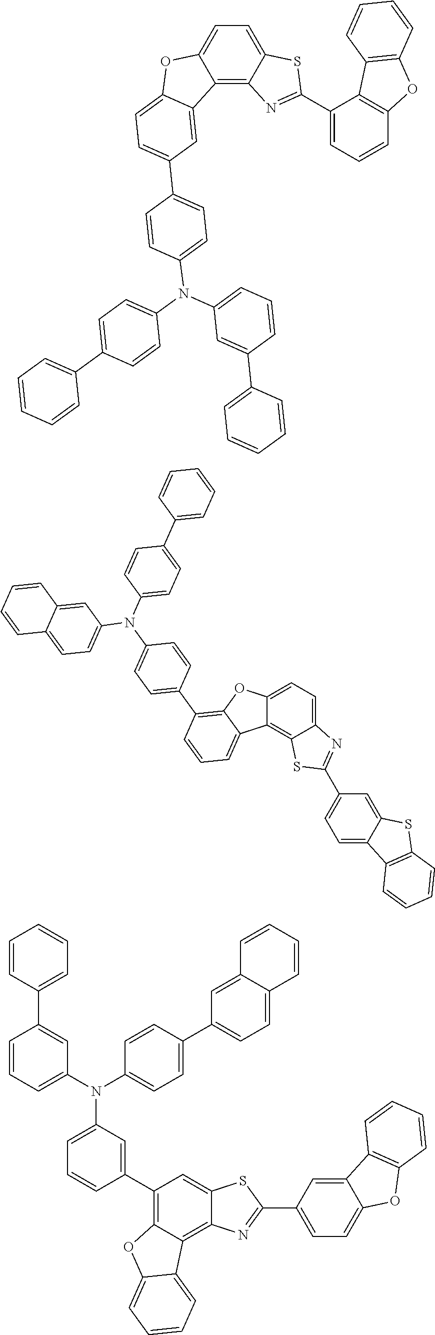

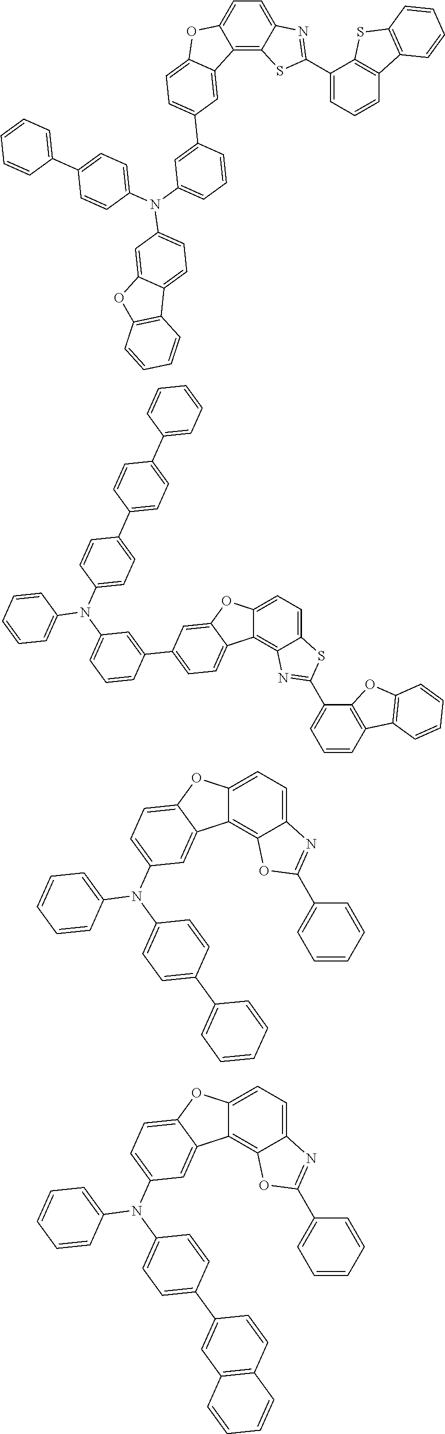

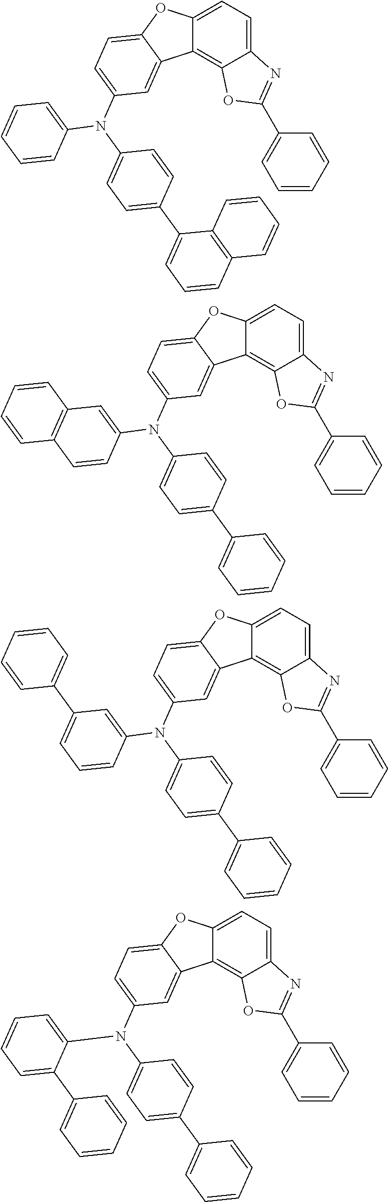

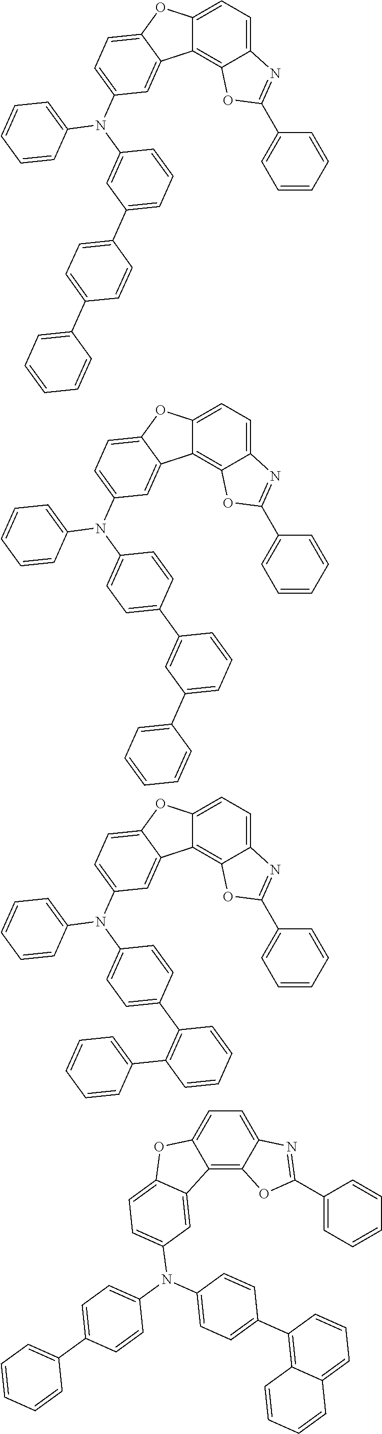

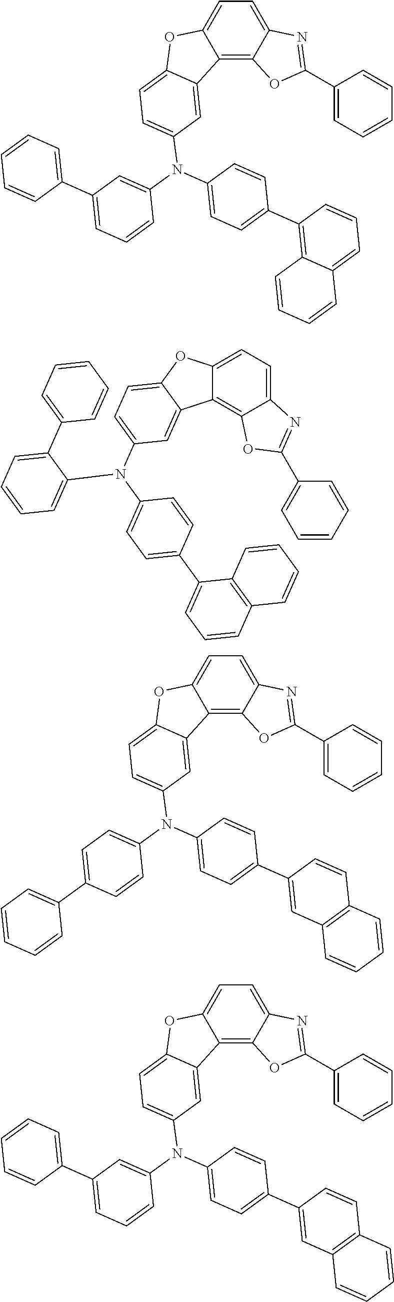

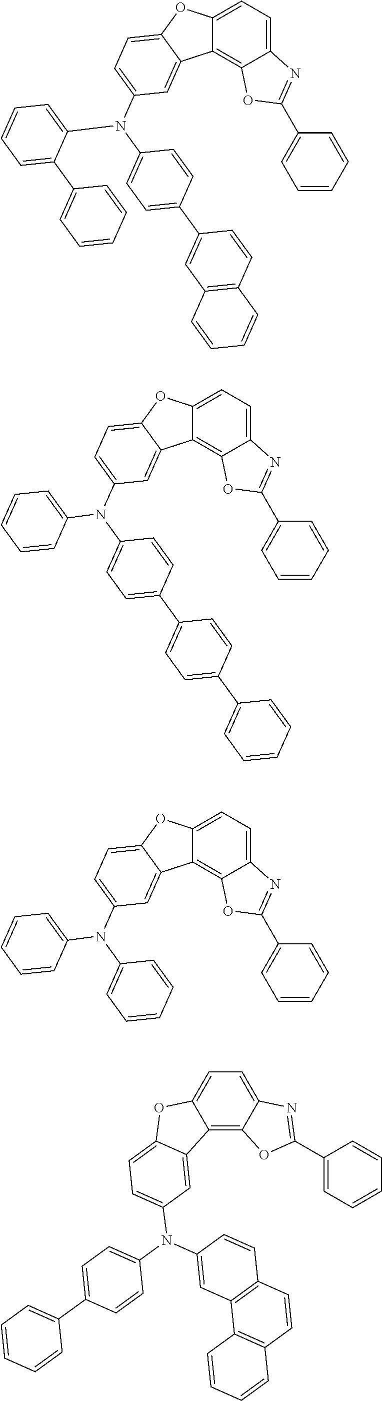

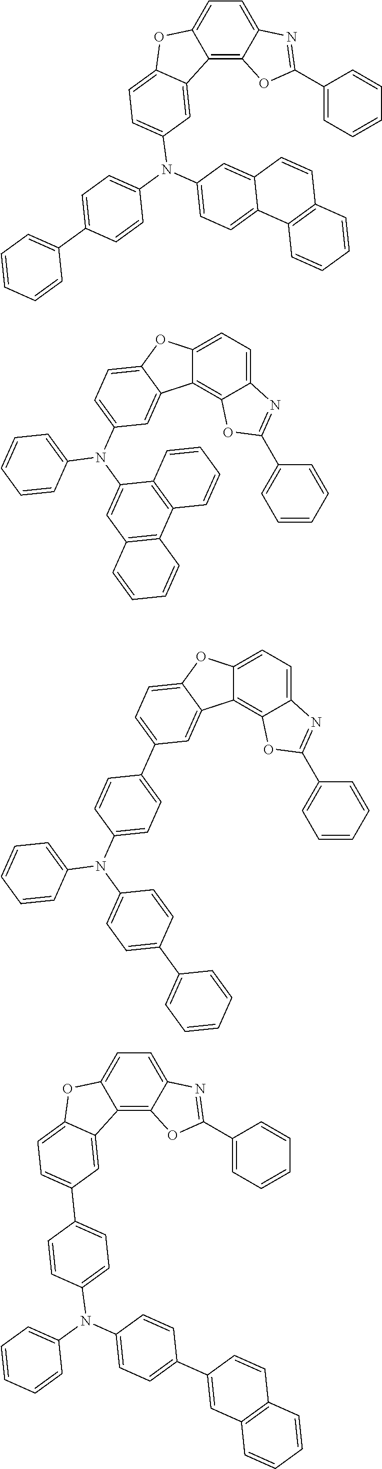

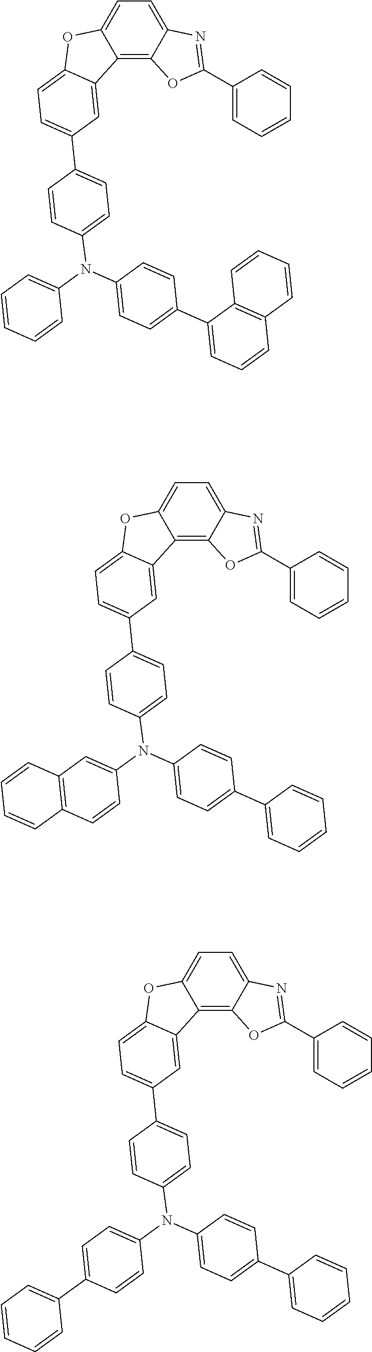 [SMILES: c1ccc(-c2nc3ccc4oc5ccc(-c6ccc(N(c7ccccc7)c7ccc(-c8cccc9ccccc89)cc7)cc6)cc5c4c3o2)cc1 ; c1ccc(-c2ccc(N(c3ccc(-c4ccc5oc6ccc7nc(-c8ccccc8)oc7c6c5c4)cc3)c3ccc4ccccc4c3)cc2)cc1 ; c1ccc(-c2ccc(N(c3ccc(-c4ccccc4)cc3)c3ccc(-c4ccc5oc6ccc7nc(-c8ccccc8)oc7c6c5c4)cc3)cc2)cc1]

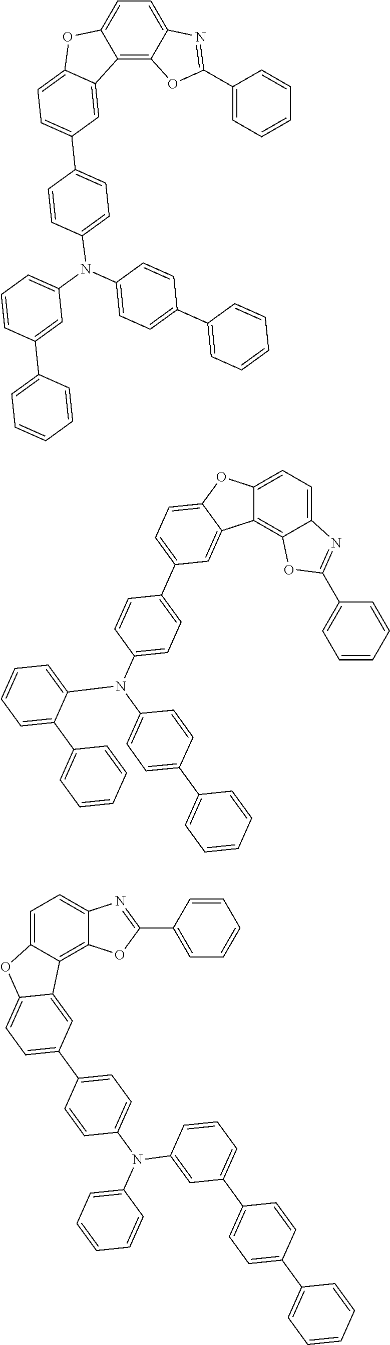 [SMILES: c1ccc(-c2ccc(N(c3ccc(-c4ccc5oc6ccc7nc(-c8ccccc8)oc7c6c5c4)cc3)c3cccc(-c4ccccc4)c3)cc2)cc1 ; c1ccc(-c2ccc(N(c3ccc(-c4ccc5oc6ccc7nc(-c8ccccc8)oc7c6c5c4)cc3)c3ccccc3-c3ccccc3)cc2)cc1 ; c1ccc(-c2ccc(-c3cccc(N(c4ccccc4)c4ccc(-c5ccc6oc7ccc8nc(-c9ccccc9)oc8c7c6c5)cc4)c3)cc2)cc1]

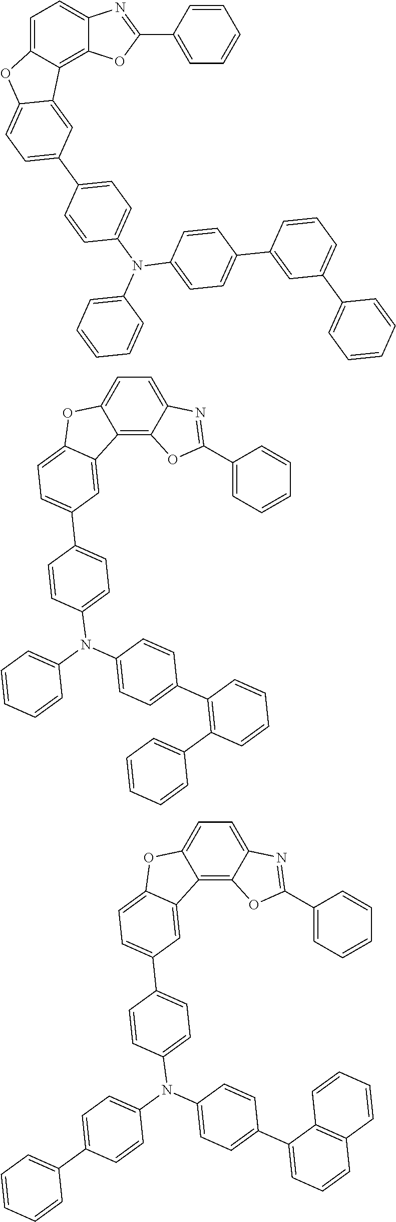

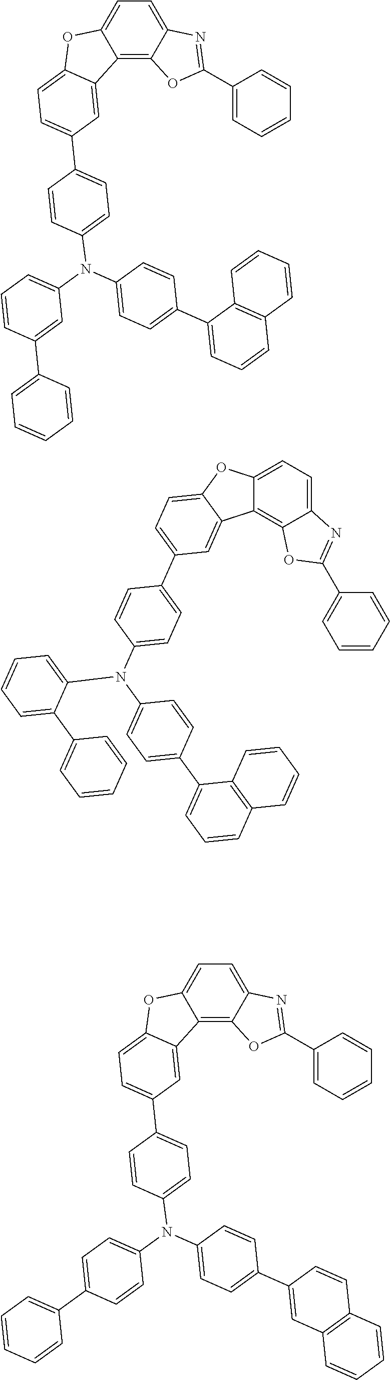 [SMILES: c1ccc(-c2cccc(N(c3ccc(-c4ccc5oc6ccc7nc(-c8ccccc8)oc7c6c5c4)cc3)c3ccc(-c4cccc5ccccc45)cc3)c2)cc1 ; c1ccc(-c2nc3ccc4oc5ccc(-c6ccc(N(c7ccc(-c8cccc9ccccc89)cc7)c7ccccc7-c7ccccc7)cc6)cc5c4c3o2)cc1 ; c1ccc(-c2ccc(N(c3ccc(-c4ccc5ccccc5c4)cc3)c3ccc(-c4ccc5oc6ccc7nc(-c8ccccc8)oc7c6c5c4)cc3)cc2)cc1]

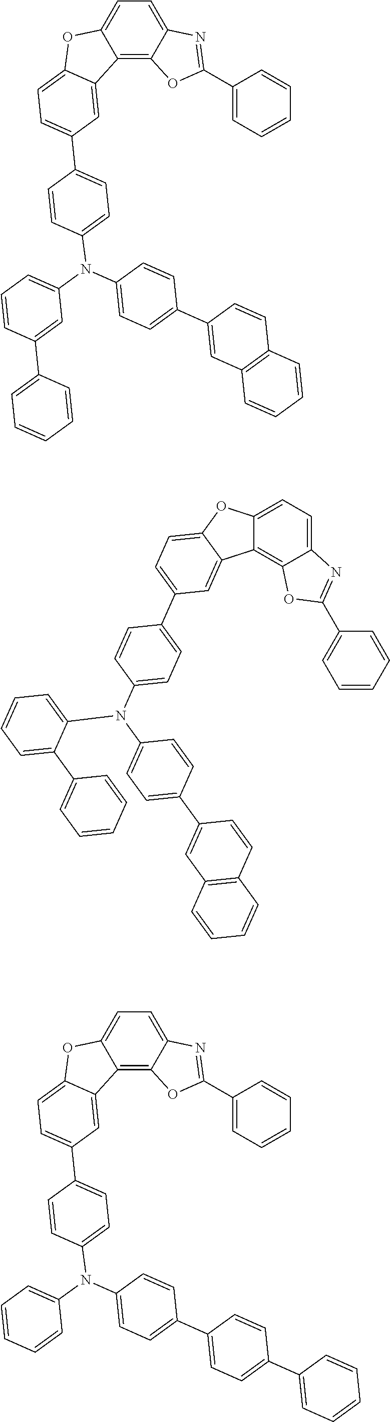 [SMILES: c1ccc(-c2cccc(N(c3ccc(-c4ccc5ccccc5c4)cc3)c3ccc(-c4ccc5oc6ccc7nc(-c8ccccc8)oc7c6c5c4)cc3)c2)cc1 ; c1ccc(-c2nc3ccc4oc5ccc(-c6ccc(N(c7ccc(-c8ccc9ccccc9c8)cc7)c7ccccc7-c7ccccc7)cc6)cc5c4c3o2)cc1 ; c1ccc(-c2ccc(-c3ccc(N(c4ccccc4)c4ccc(-c5ccc6oc7ccc8nc(-c9ccccc9)oc8c7c6c5)cc4)cc3)cc2)cc1]

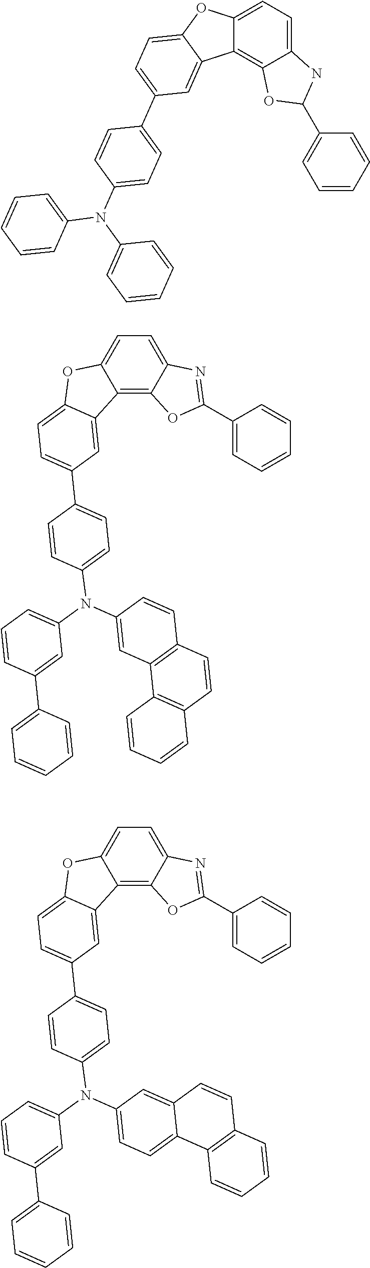

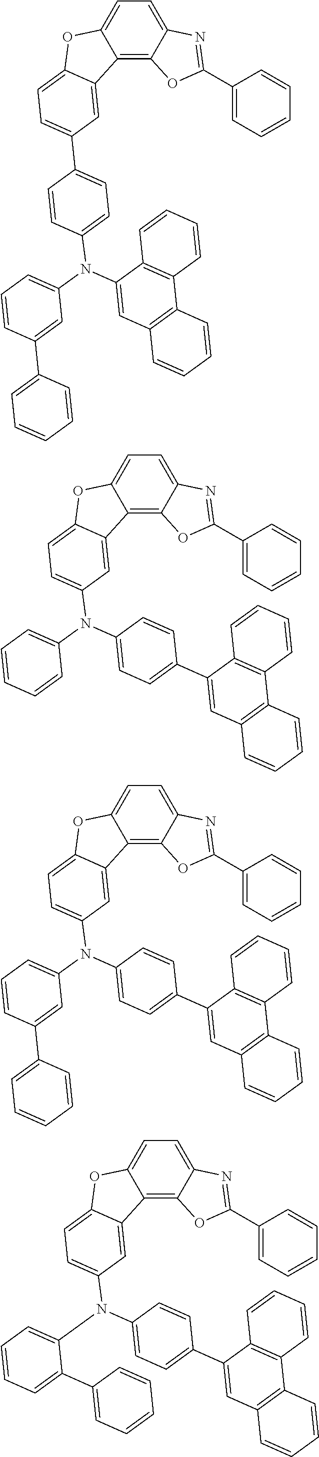 [SMILES: c1ccc(-c2cccc(N(c3ccc(-c4ccc5oc6ccc7nc(-c8ccccc8)oc7c6c5c4)cc3)c3cc4ccccc4c4ccccc34)c2)cc1 ; c1ccc(-c2nc3ccc4oc5ccc(N(c6ccccc6)c6ccc(-c7cc8ccccc8c8ccccc78)cc6)cc5c4c3o2)cc1 ; c1ccc(-c2cccc(N(c3ccc(-c4cc5ccccc5c5ccccc45)cc3)c3ccc4oc5ccc6nc(-c7ccccc7)oc6c5c4c3)c2)cc1 ; c1ccc(-c2nc3ccc4oc5ccc(N(c6ccc(-c7cc8ccccc8c8ccccc78)cc6)c6ccccc6-c6ccccc6)cc5c4c3o2)cc1]

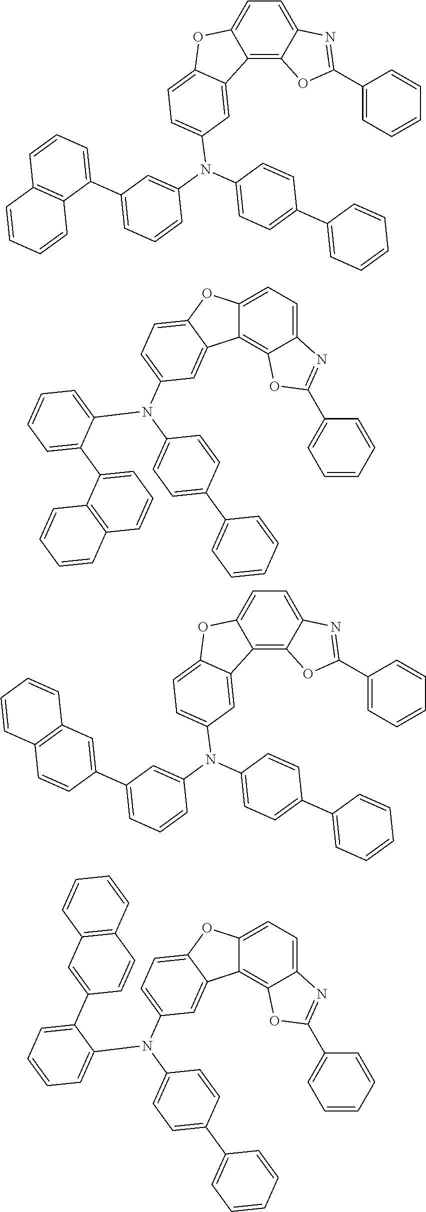

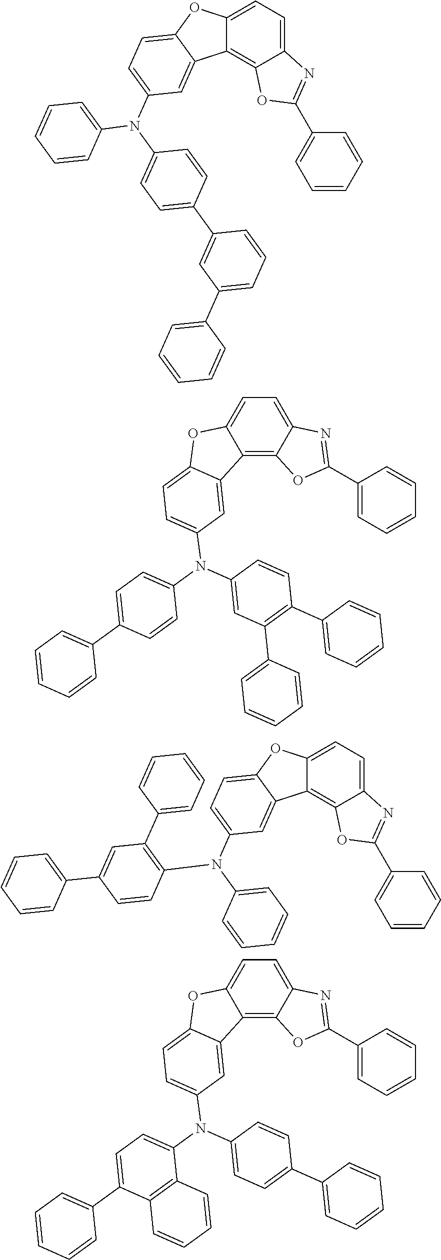

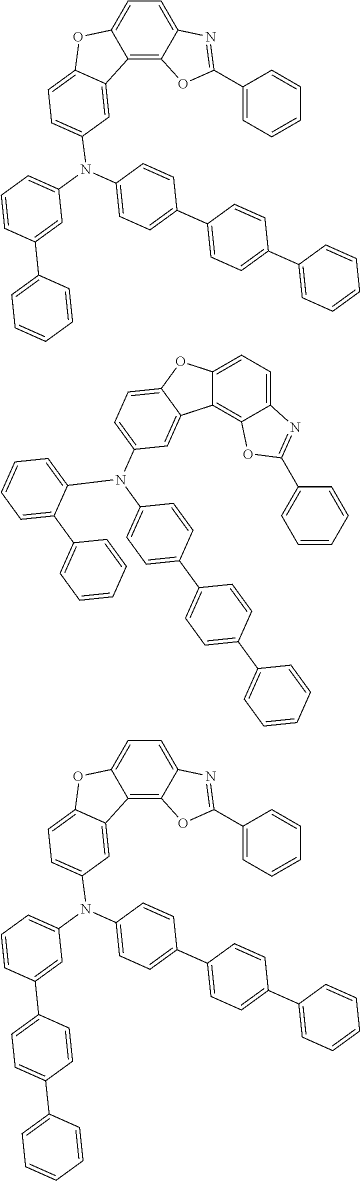 [SMILES: c1ccc(-c2ccc(-c3ccc(N(c4cccc(-c5ccccc5)c4)c4ccc5oc6ccc7nc(-c8ccccc8)oc7c6c5c4)cc3)cc2)cc1 ; c1ccc(-c2ccc(-c3ccc(N(c4ccc5oc6ccc7nc(-c8ccccc8)oc7c6c5c4)c4ccccc4-c4ccccc4)cc3)cc2)cc1 ; c1ccc(-c2ccc(-c3ccc(N(c4cccc(-c5ccc(-c6ccccc6)cc5)c4)c4ccc5oc6ccc7nc(-c8ccccc8)oc7c6c5c4)cc3)cc2)cc1]

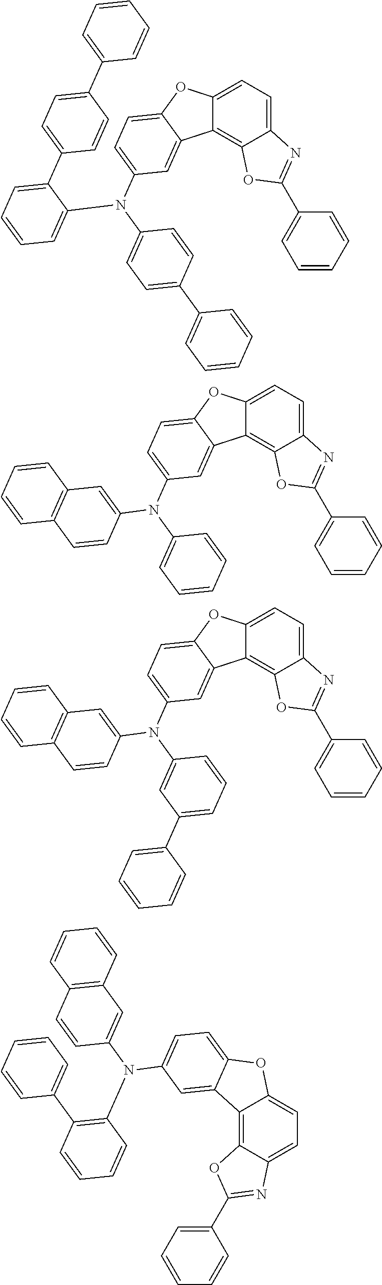

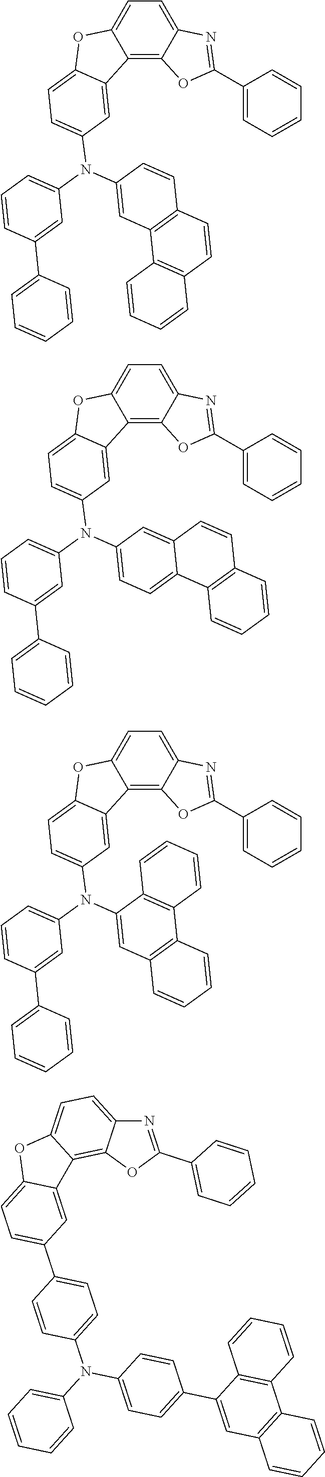

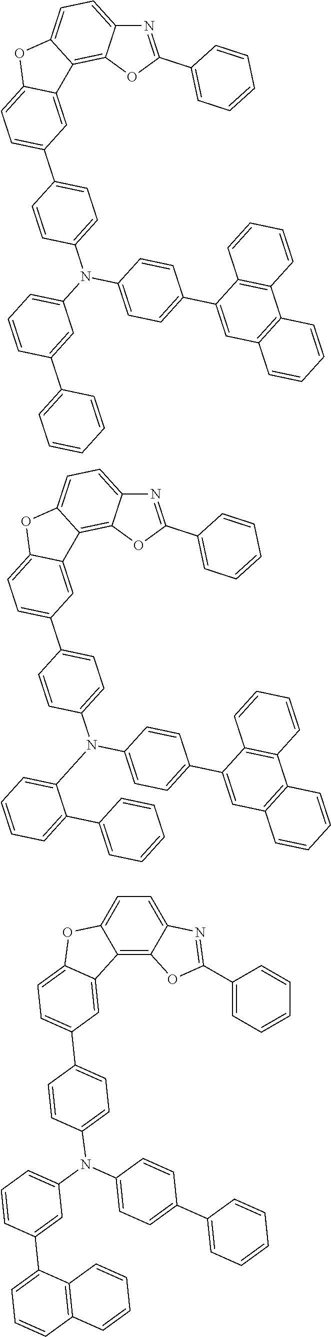

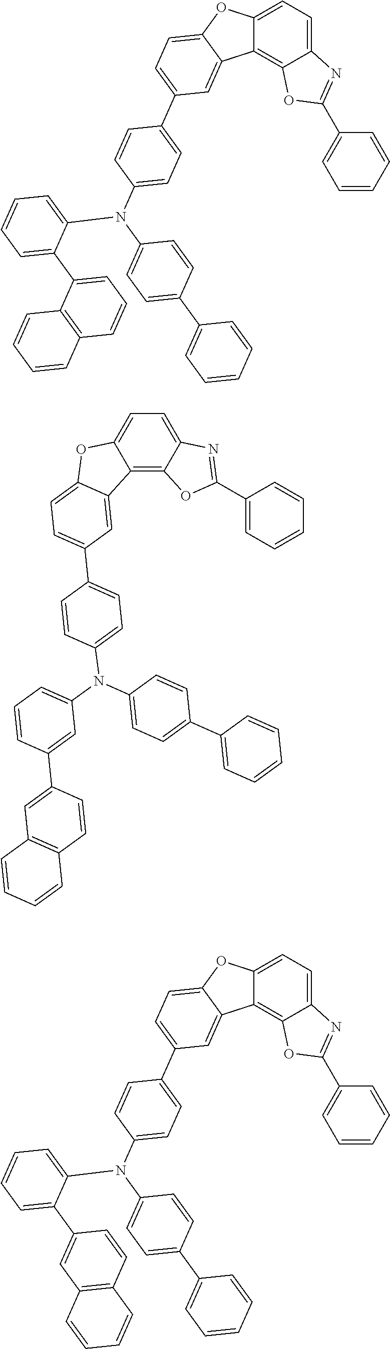 [SMILES: c1ccc(-c2ccc(N(c3ccc(-c4ccc5oc6ccc7nc(-c8ccccc8)oc7c6c5c4)cc3)c3ccccc3-c3cccc4ccccc34)cc2)cc1 ; c1ccc(-c2ccc(N(c3ccc(-c4ccc5oc6ccc7nc(-c8ccccc8)oc7c6c5c4)cc3)c3cccc(-c4ccc5ccccc5c4)c3)cc2)cc1 ; c1ccc(-c2ccc(N(c3ccc(-c4ccc5oc6ccc7nc(-c8ccccc8)oc7c6c5c4)cc3)c3ccccc3-c3ccc4ccccc4c3)cc2)cc1]

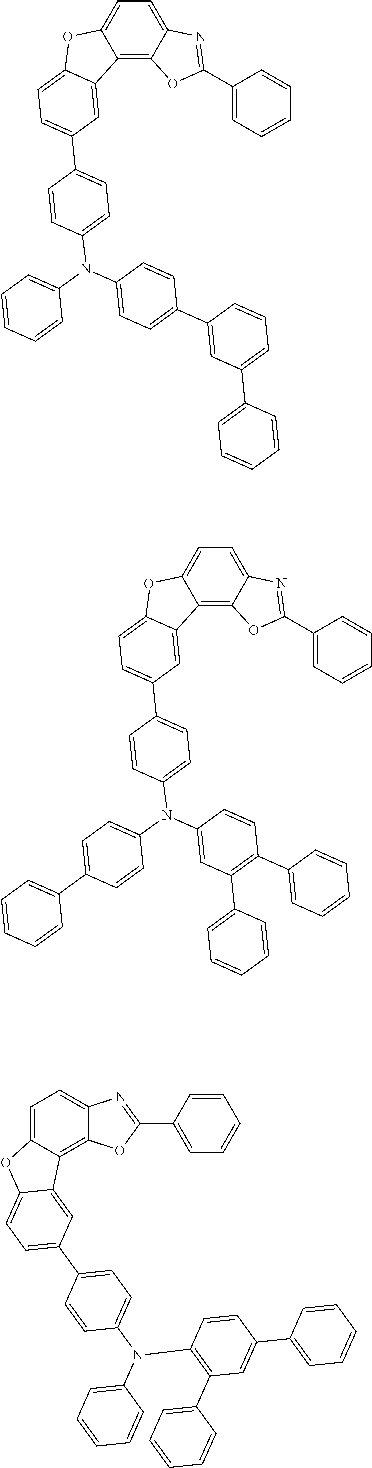 [SMILES: c1ccc(-c2cccc(-c3ccc(N(c4ccccc4)c4ccc(-c5ccc6oc7ccc8nc(-c9ccccc9)oc8c7c6c5)cc4)cc3)c2)cc1 ; c1ccc(-c2ccc(N(c3ccc(-c4ccc5oc6ccc7nc(-c8ccccc8)oc7c6c5c4)cc3)c3ccc(-c4ccccc4)c(-c4ccccc4)c3)cc2)cc1 ; c1ccc(-c2ccc(N(c3ccccc3)c3ccc(-c4ccc5oc6ccc7nc(-c8ccccc8)oc7c6c5c4)cc3)c(-c3ccccc3)c2)cc1]

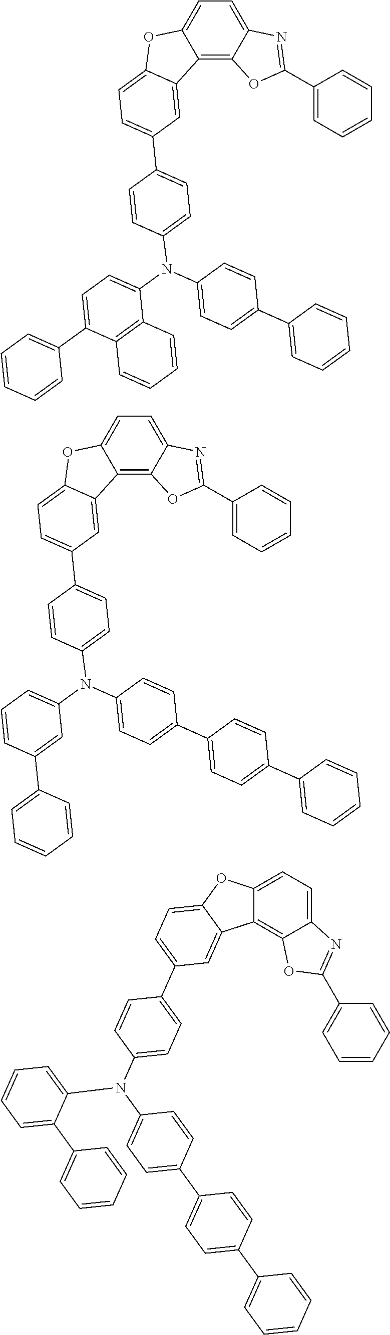

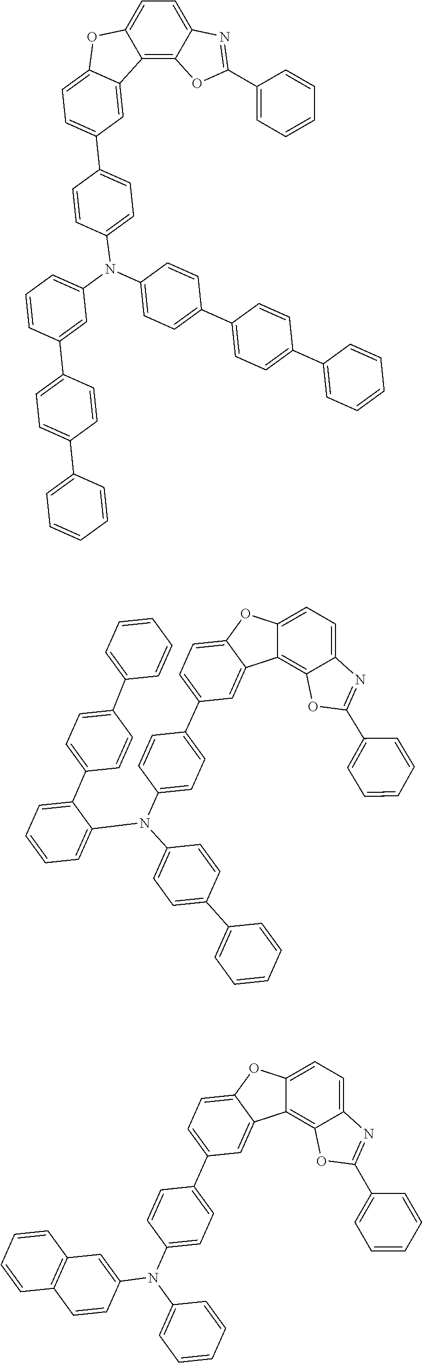 [SMILES: c1ccc(-c2ccc(-c3ccc(N(c4ccc(-c5ccc6oc7ccc8nc(-c9ccccc9)oc8c7c6c5)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4ccc5oc6ccc7nc(-c8ccccc8)oc7c6c5c4)cc3)cc2)cc1 ; c1ccc(-c2nc3ccc4oc5ccc(-c6ccc(N(c7ccccc7)c7ccc8ccccc8c7)cc6)cc5c4c3o2)cc1]

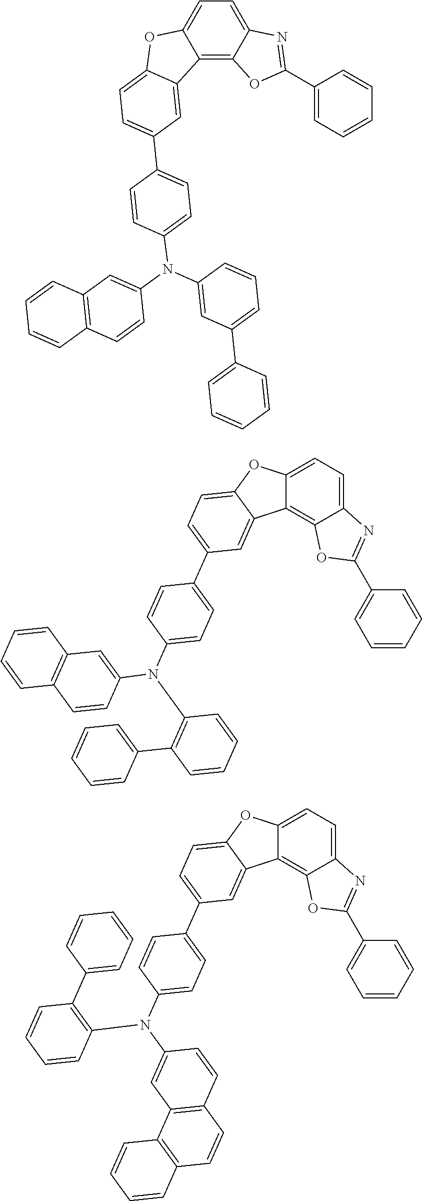

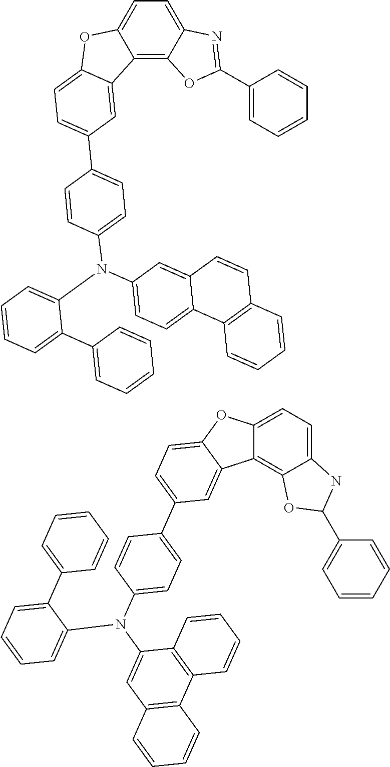

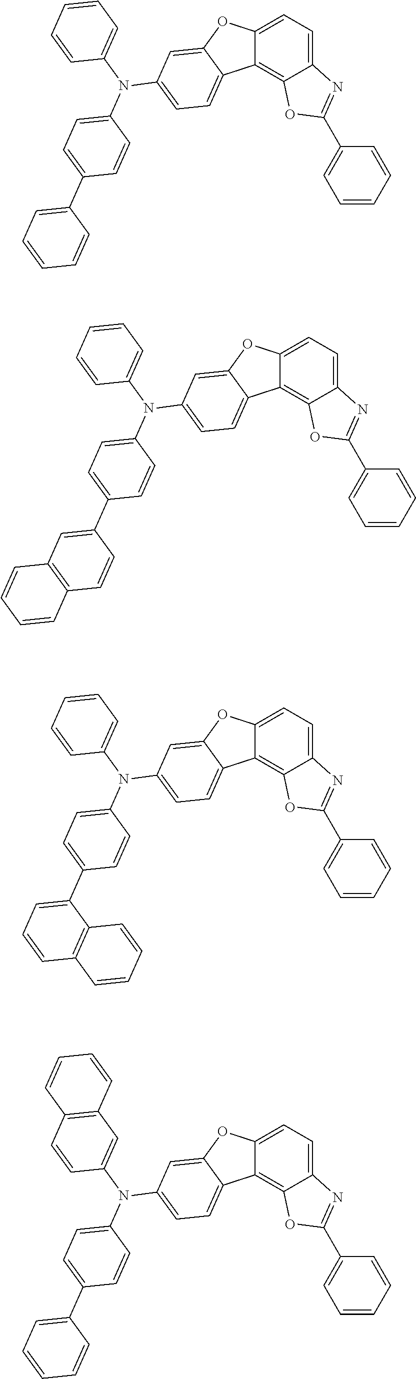 [SMILES: c1ccc(-c2ccc(N(c3ccccc3)c3ccc4c(c3)oc3ccc5nc(-c6ccccc6)oc5c34)cc2)cc1 ; c1ccc(-c2nc3ccc4oc5cc(N(c6ccccc6)c6ccc(-c7ccc8ccccc8c7)cc6)ccc5c4c3o2)cc1 ; c1ccc(-c2nc3ccc4oc5cc(N(c6ccccc6)c6ccc(-c7cccc8ccccc78)cc6)ccc5c4c3o2)cc1 ; c1ccc(-c2ccc(N(c3ccc4ccccc4c3)c3ccc4c(c3)oc3ccc5nc(-c6ccccc6)oc5c34)cc2)cc1]

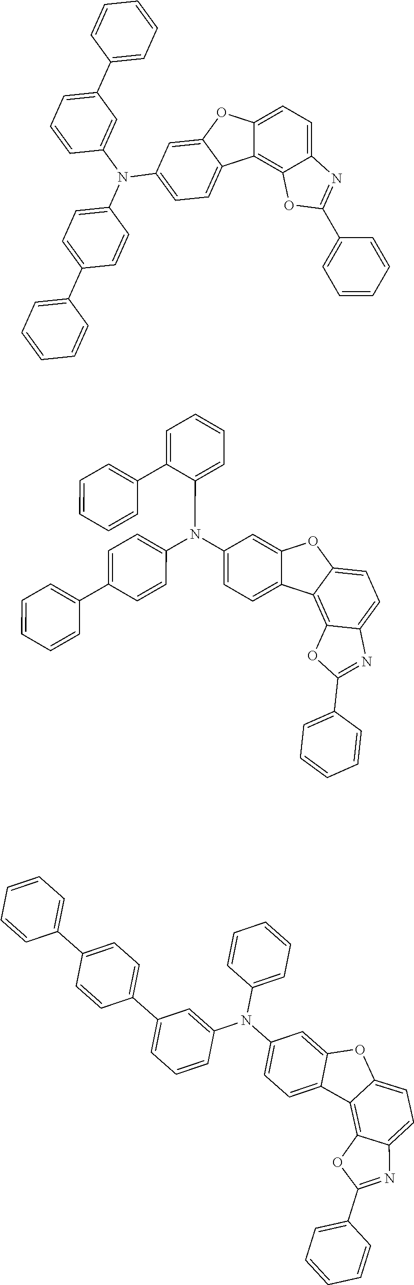 [SMILES: c1ccc(-c2ccc(N(c3cccc(-c4ccccc4)c3)c3ccc4c(c3)oc3ccc5nc(-c6ccccc6)oc5c34)cc2)cc1 ; c1ccc(-c2ccc(N(c3ccc4c(c3)oc3ccc5nc(-c6ccccc6)oc5c34)c3ccccc3-c3ccccc3)cc2)cc1 ; c1ccc(-c2ccc(-c3cccc(N(c4ccccc4)c4ccc5c(c4)oc4ccc6nc(-c7ccccc7)oc6c45)c3)cc2)cc1]

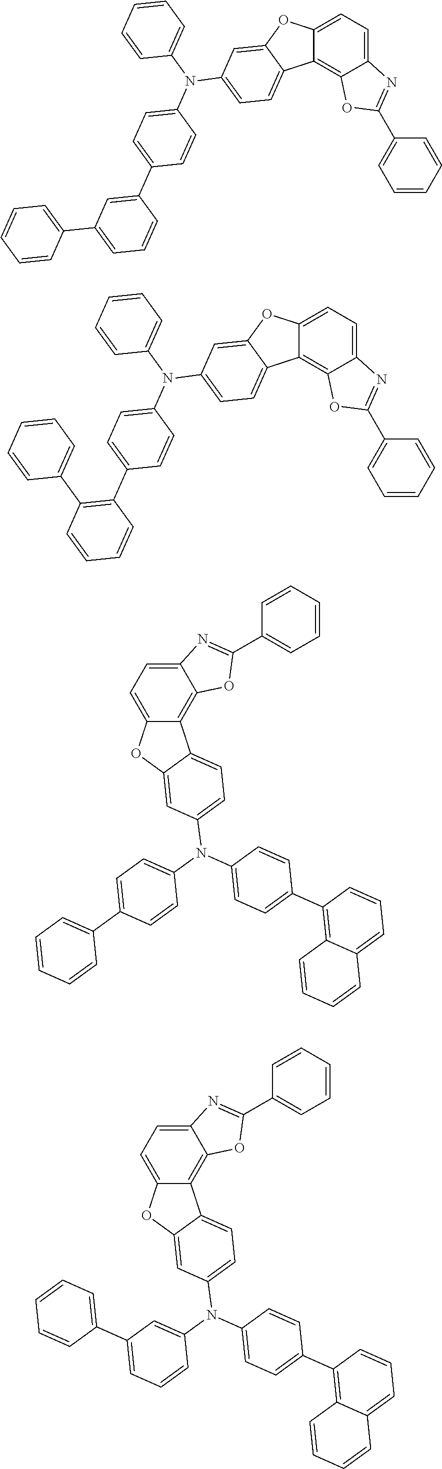

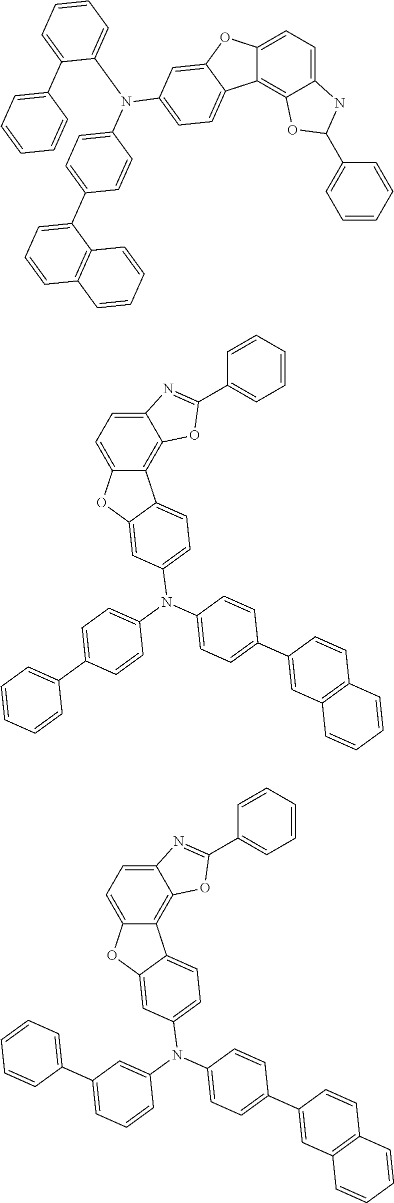

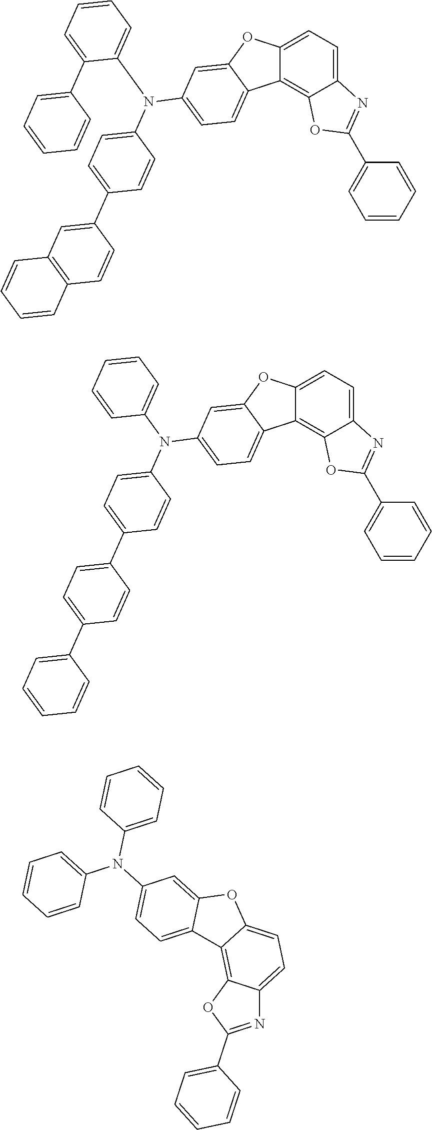 [SMILES: c1ccc(-c2nc3ccc4oc5cc(N(c6ccc(-c7ccc8ccccc8c7)cc6)c6ccccc6-c6ccccc6)ccc5c4c3o2)cc1 ; c1ccc(-c2ccc(-c3ccc(N(c4ccccc4)c4ccc5c(c4)oc4ccc6nc(-c7ccccc7)oc6c45)cc3)cc2)cc1 ; c1ccc(-c2nc3ccc4oc5cc(N(c6ccccc6)c6ccccc6)ccc5c4c3o2)cc1]

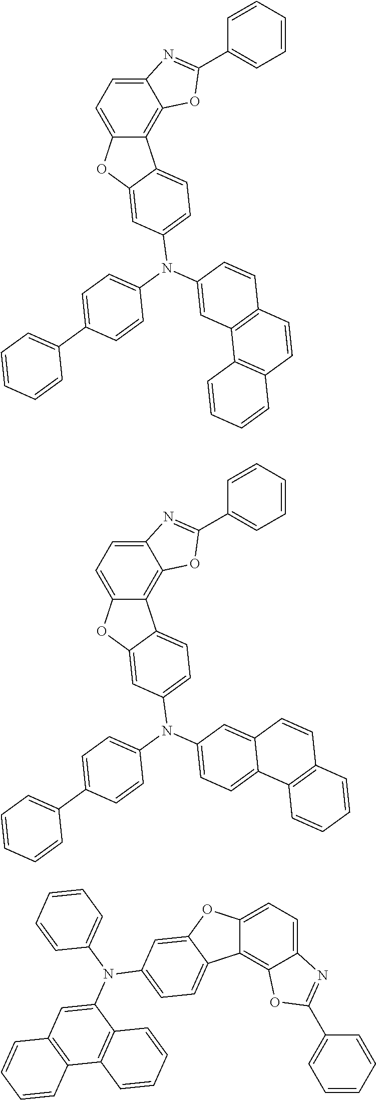

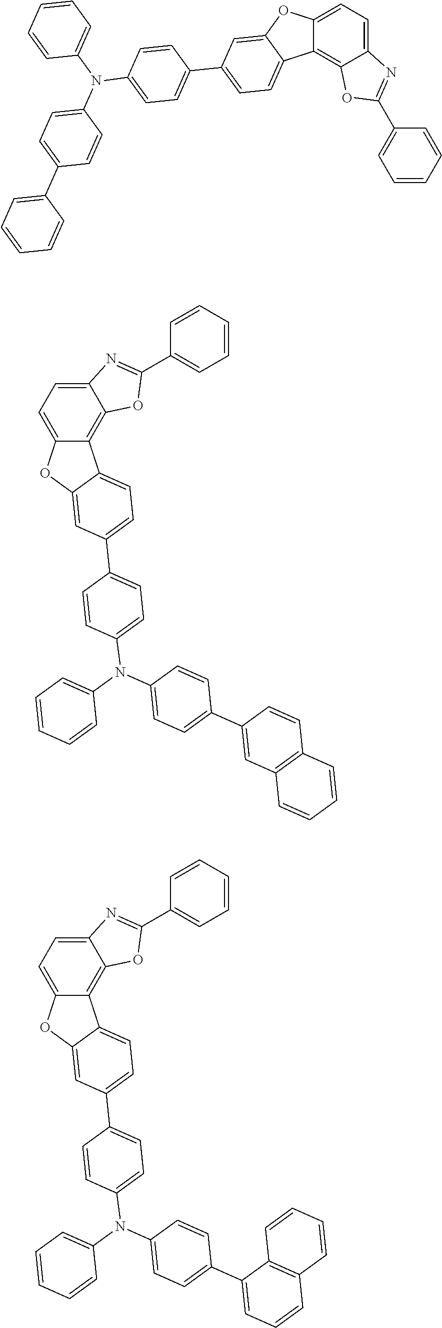 [SMILES: c1ccc(-c2ccc(N(c3ccccc3)c3ccc(-c4ccc5c(c4)oc4ccc6nc(-c7ccccc7)oc6c45)cc3)cc2)cc1 ; c1ccc(-c2nc3ccc4oc5cc(-c6ccc(N(c7ccccc7)c7ccc(-c8ccc9ccccc9c8)cc7)cc6)ccc5c4c3o2)cc1 ; c1ccc(-c2nc3ccc4oc5cc(-c6ccc(N(c7ccccc7)c7ccc(-c8cccc9ccccc89)cc7)cc6)ccc5c4c3o2)cc1]

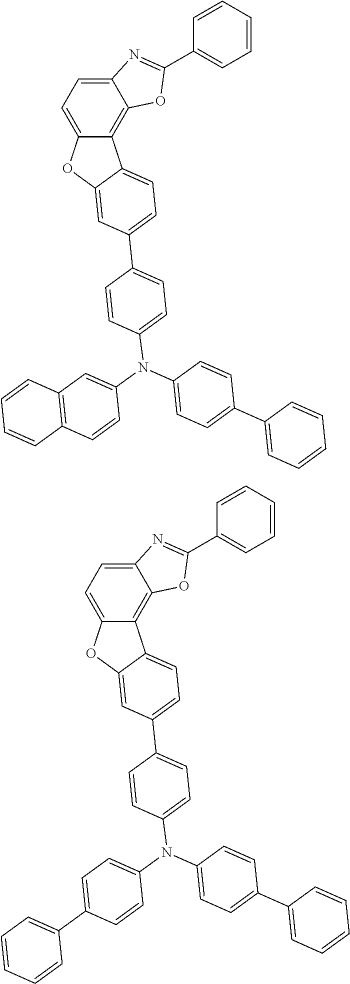

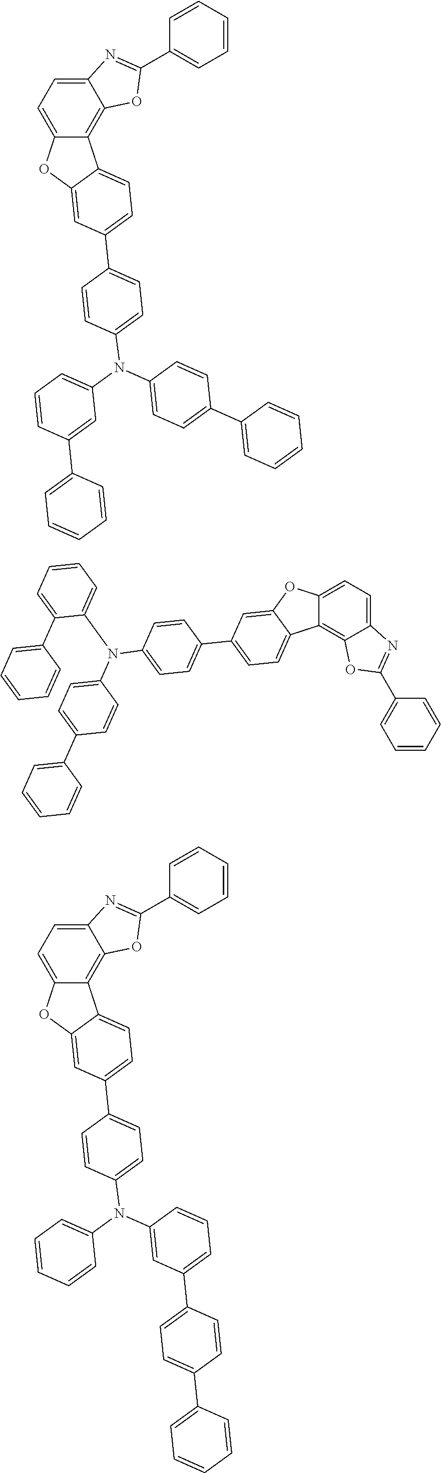

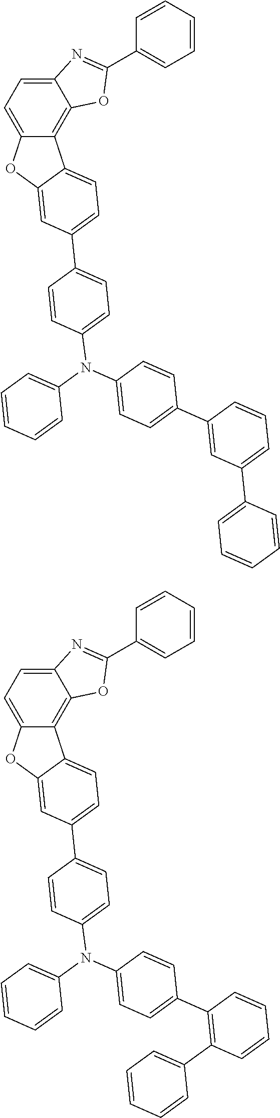 [SMILES: c1ccc(-c2cccc(-c3ccc(N(c4ccccc4)c4ccc(-c5ccc6c(c5)oc5ccc7nc(-c8ccccc8)oc7c56)cc4)cc3)c2)cc1 ; c1ccc(-c2nc3ccc4oc5cc(-c6ccc(N(c7ccccc7)c7ccc(-c8ccccc8-c8ccccc8)cc7)cc6)ccc5c4c3o2)cc1]

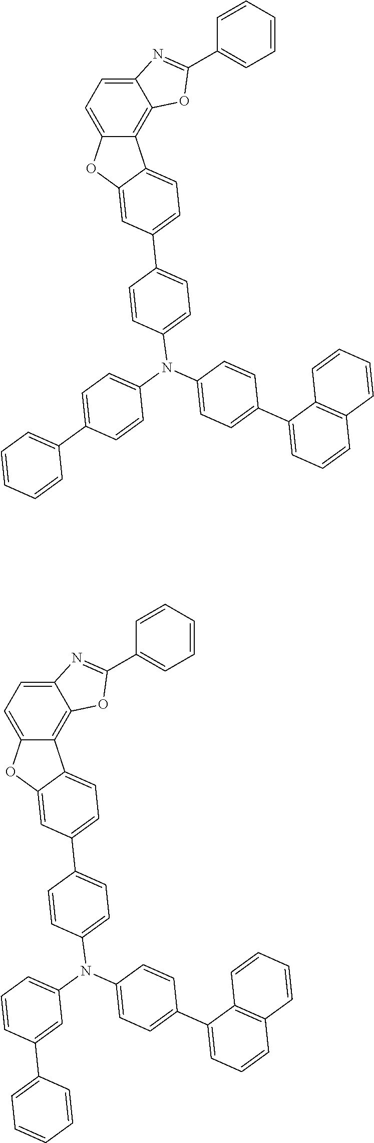 [SMILES: c1ccc(-c2ccc(N(c3ccc(-c4ccc5c(c4)oc4ccc6nc(-c7ccccc7)oc6c45)cc3)c3ccc(-c4cccc5ccccc45)cc3)cc2)cc1 ; c1ccc(-c2cccc(N(c3ccc(-c4ccc5c(c4)oc4ccc6nc(-c7ccccc7)oc6c45)cc3)c3ccc(-c4cccc5ccccc45)cc3)c2)cc1]

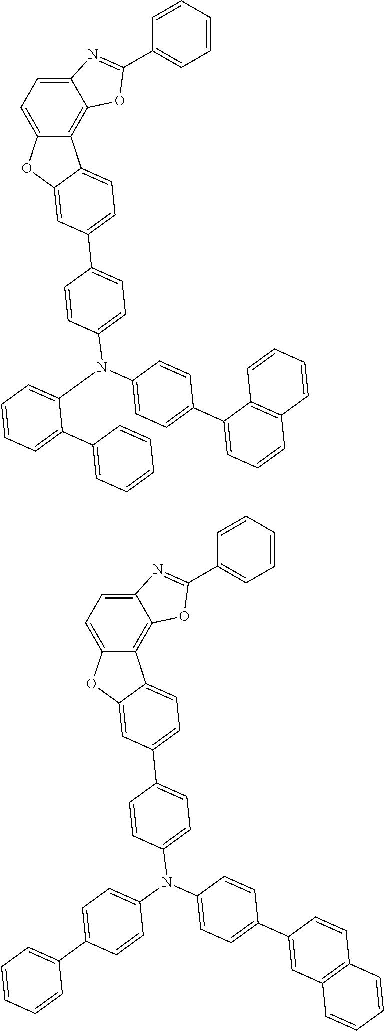 [SMILES: c1ccc(-c2nc3ccc4oc5cc(-c6ccc(N(c7ccc(-c8cccc9ccccc89)cc7)c7ccccc7-c7ccccc7)cc6)ccc5c4c3o2)cc1 ; c1ccc(-c2ccc(N(c3ccc(-c4ccc5ccccc5c4)cc3)c3ccc(-c4ccc5c(c4)oc4ccc6nc(-c7ccccc7)oc6c45)cc3)cc2)cc1]

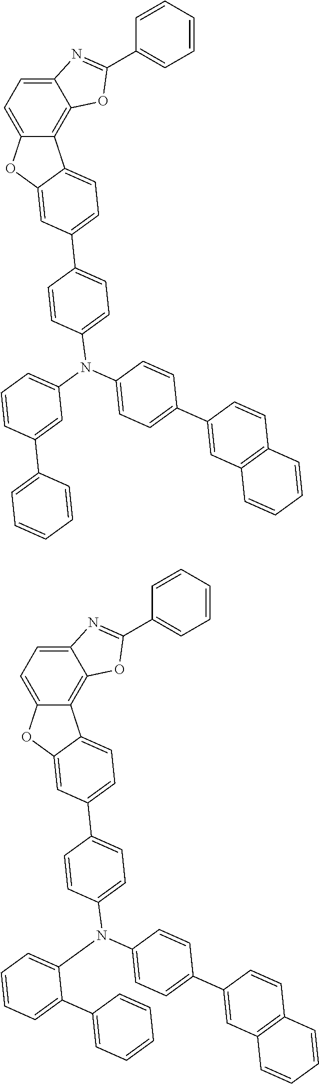 [SMILES: c1ccc(-c2cccc(N(c3ccc(-c4ccc5ccccc5c4)cc3)c3ccc(-c4ccc5c(c4)oc4ccc6nc(-c7ccccc7)oc6c45)cc3)c2)cc1 ; c1ccc(-c2nc3ccc4oc5cc(-c6ccc(N(c7ccc(-c8ccc9ccccc9c8)cc7)c7ccccc7-c7ccccc7)cc6)ccc5c4c3o2)cc1]

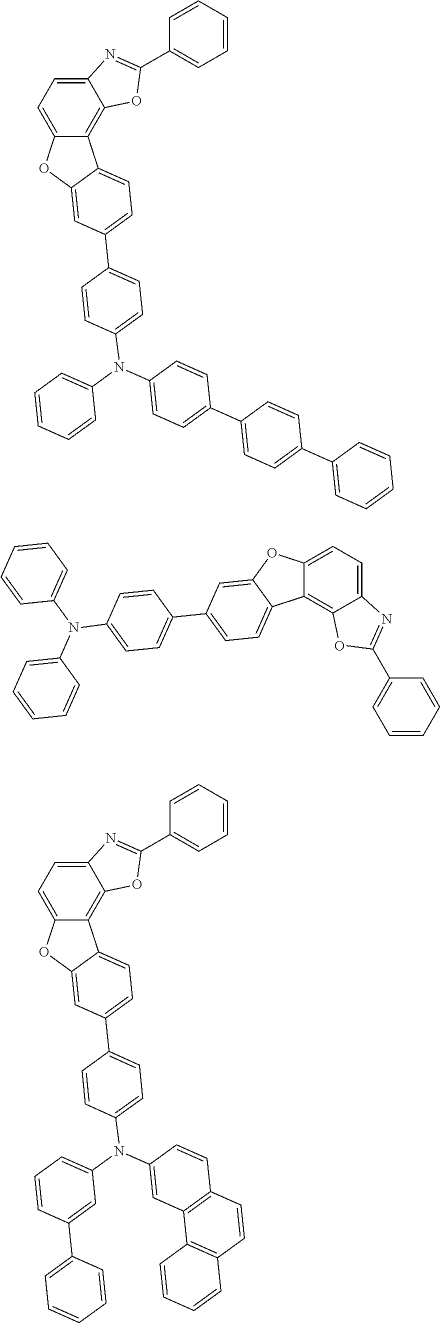 [SMILES: c1ccc(-c2ccc(-c3ccc(N(c4ccccc4)c4ccc(-c5ccc6c(c5)oc5ccc7nc(-c8ccccc8)oc7c56)cc4)cc3)cc2)cc1 ; c1ccc(-c2nc3ccc4oc5cc(-c6ccc(N(c7ccccc7)c7ccccc7)cc6)ccc5c4c3o2)cc1 ; c1ccc(-c2cccc(N(c3ccc(-c4ccc5c(c4)oc4ccc6nc(-c7ccccc7)oc6c45)cc3)c3ccc4ccc5ccccc5c4c3)c2)cc1]

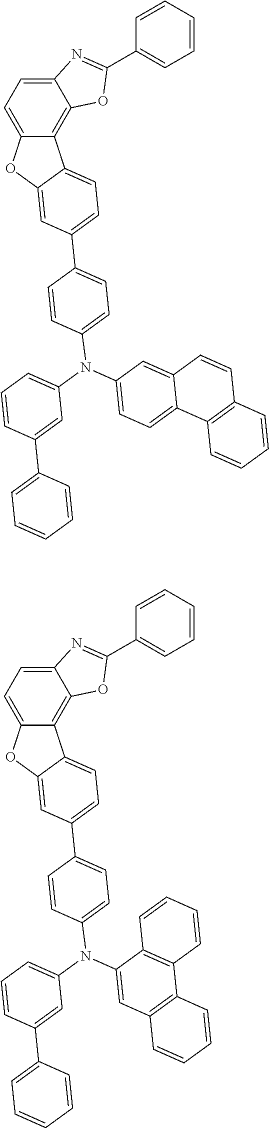 [SMILES: c1ccc(-c2cccc(N(c3ccc(-c4ccc5c(c4)oc4ccc6nc(-c7ccccc7)oc6c45)cc3)c3ccc4c(ccc5ccccc54)c3)c2)cc1 ; c1ccc(-c2cccc(N(c3ccc(-c4ccc5c(c4)oc4ccc6nc(-c7ccccc7)oc6c45)cc3)c3cc4ccccc4c4ccccc34)c2)cc1]

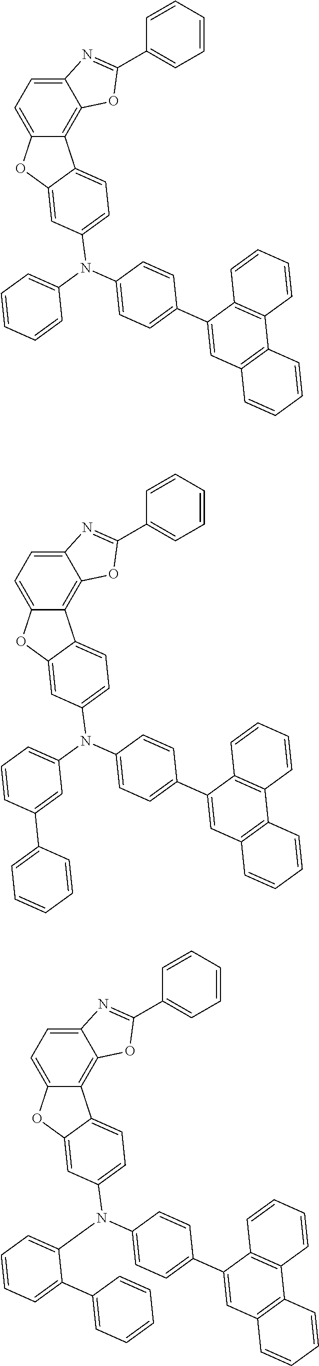 [SMILES: c1ccc(-c2nc3ccc4oc5cc(N(c6ccccc6)c6ccc(-c7cc8ccccc8c8ccccc78)cc6)ccc5c4c3o2)cc1 ; c1ccc(-c2cccc(N(c3ccc(-c4cc5ccccc5c5ccccc45)cc3)c3ccc4c(c3)oc3ccc5nc(-c6ccccc6)oc5c34)c2)cc1 ; c1ccc(-c2nc3ccc4oc5cc(N(c6ccc(-c7cc8ccccc8c8ccccc78)cc6)c6ccccc6-c6ccccc6)ccc5c4c3o2)cc1]

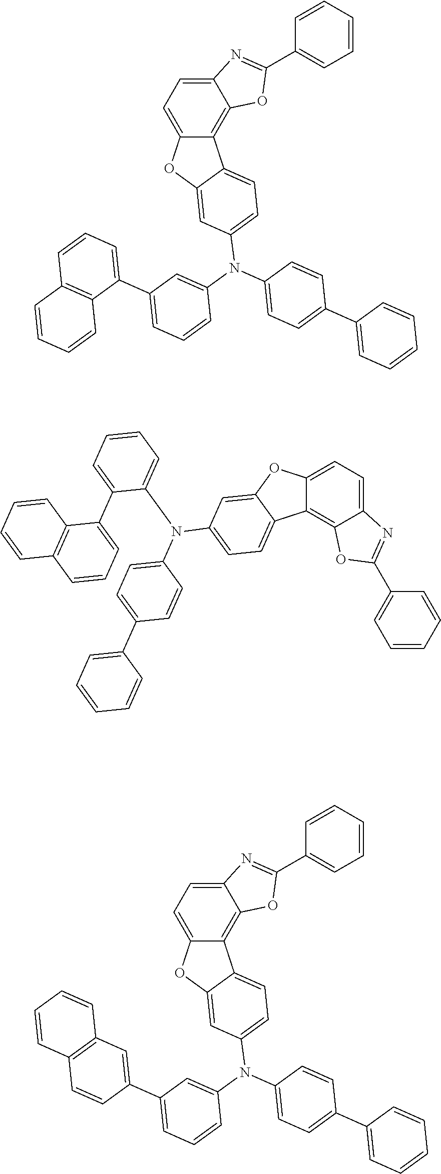 [SMILES: c1ccc(-c2ccc(N(c3cccc(-c4cccc5ccccc45)c3)c3ccc4c(c3)oc3ccc5nc(-c6ccccc6)oc5c34)cc2)cc1 ; c1ccc(-c2ccc(N(c3ccc4c(c3)oc3ccc5nc(-c6ccccc6)oc5c34)c3ccccc3-c3cccc4ccccc34)cc2)cc1 ; c1ccc(-c2ccc(N(c3cccc(-c4ccc5ccccc5c4)c3)c3ccc4c(c3)oc3ccc5nc(-c6ccccc6)oc5c34)cc2)cc1]

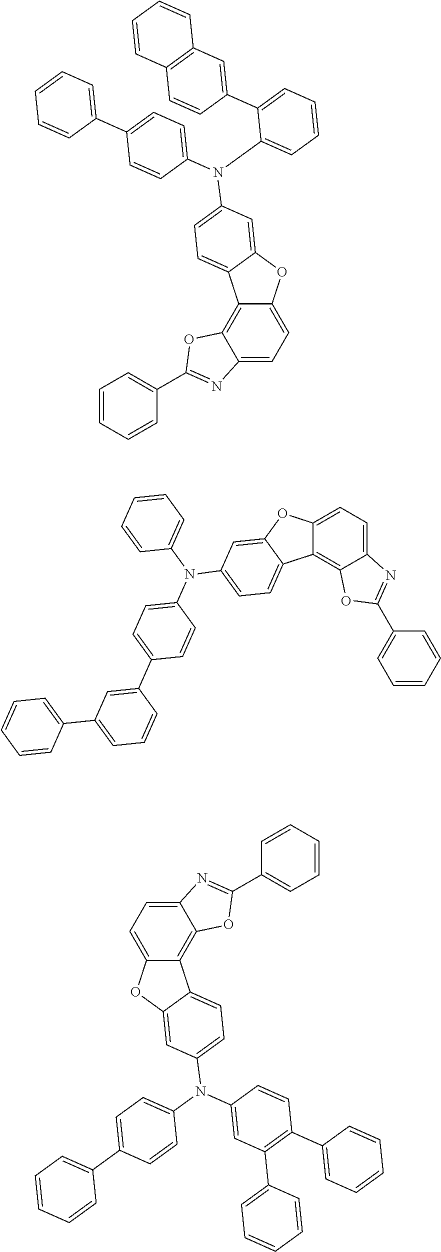 [SMILES: c1ccc(-c2ccc(N(c3ccc4c(c3)oc3ccc5nc(-c6ccccc6)oc5c34)c3ccccc3-c3ccc4ccccc4c3)cc2)cc1 ; c1ccc(-c2cccc(-c3ccc(N(c4ccccc4)c4ccc5c(c4)oc4ccc6nc(-c7ccccc7)oc6c45)cc3)c2)cc1 ; c1ccc(-c2ccc(N(c3ccc(-c4ccccc4)c(-c4ccccc4)c3)c3ccc4c(c3)oc3ccc5nc(-c6ccccc6)oc5c34)cc2)cc1]

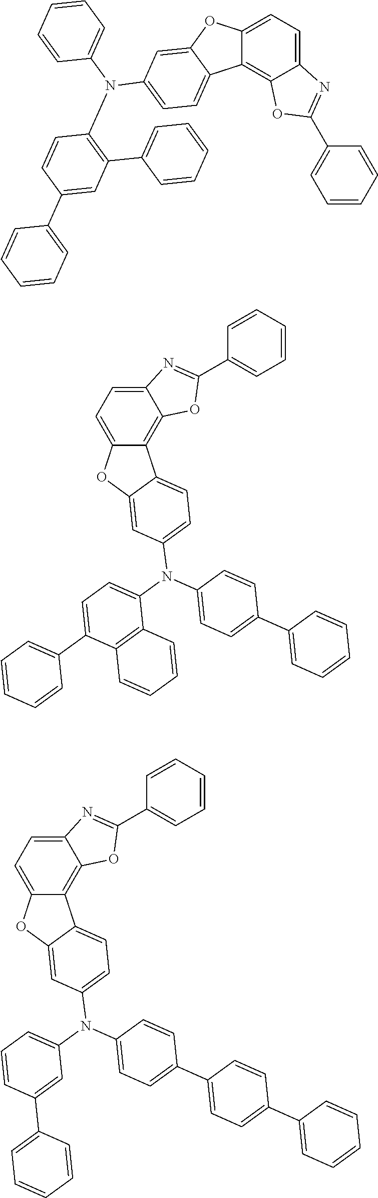 [SMILES: c1ccc(-c2ccc(N(c3ccccc3)c3ccc4c(c3)oc3ccc5nc(-c6ccccc6)oc5c34)c(-c3ccccc3)c2)cc1 ; c1ccc(-c2ccc(N(c3ccc4c(c3)oc3ccc5nc(-c6ccccc6)oc5c34)c3ccc(-c4ccccc4)c4ccccc34)cc2)cc1 ; c1ccc(-c2ccc(-c3ccc(N(c4cccc(-c5ccccc5)c4)c4ccc5c(c4)oc4ccc6nc(-c7ccccc7)oc6c45)cc3)cc2)cc1]

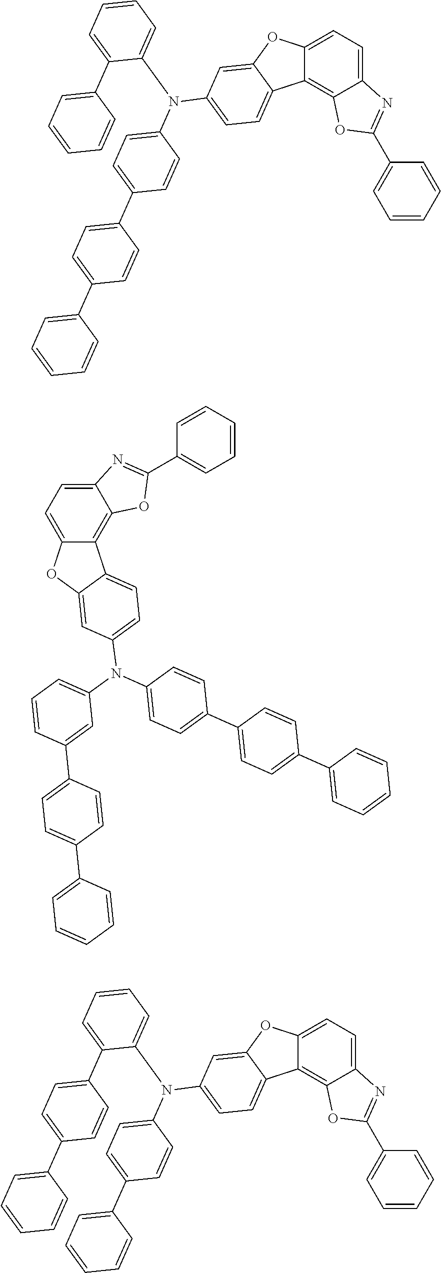 [SMILES: c1ccc(-c2ccc(-c3ccc(N(c4ccc5c(c4)oc4ccc6nc(-c7ccccc7)oc6c45)c4ccccc4-c4ccccc4)cc3)cc2)cc1 ; c1ccc(-c2ccc(-c3ccc(N(c4cccc(-c5ccc(-c6ccccc6)cc5)c4)c4ccc5c(c4)oc4ccc6nc(-c7ccccc7)oc6c45)cc3)cc2)cc1 ; c1ccc(-c2ccc(-c3ccccc3N(c3ccc(-c4ccccc4)cc3)c3ccc4c(c3)oc3ccc5nc(-c6ccccc6)oc5c34)cc2)cc1]

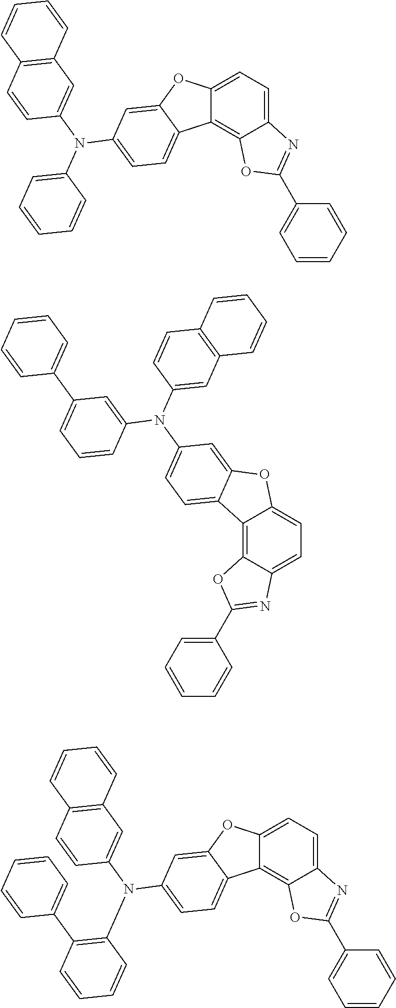 [SMILES: c1ccc(-c2nc3ccc4oc5cc(N(c6ccccc6)c6ccc7ccccc7c6)ccc5c4c3o2)cc1 ; c1ccc(-c2cccc(N(c3ccc4ccccc4c3)c3ccc4c(c3)oc3ccc5nc(-c6ccccc6)oc5c34)c2)cc1 ; c1ccc(-c2nc3ccc4oc5cc(N(c6ccc7ccccc7c6)c6ccccc6-c6ccccc6)ccc5c4c3o2)cc1]

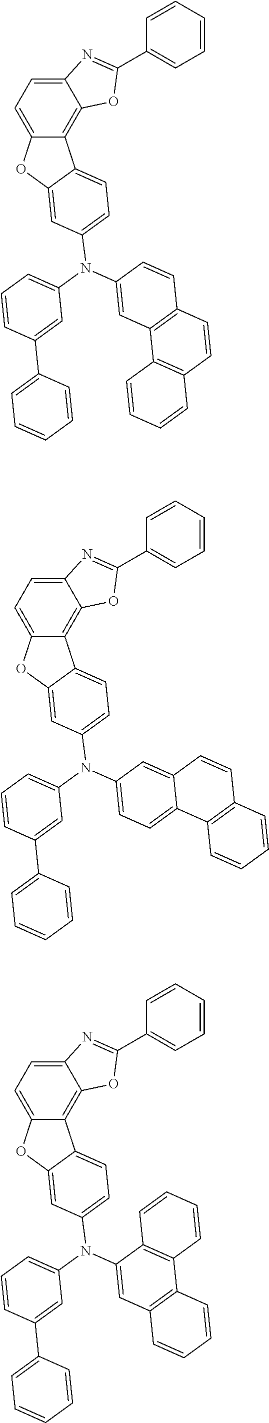 [SMILES: c1ccc(-c2cccc(N(c3ccc4c(c3)oc3ccc5nc(-c6ccccc6)oc5c34)c3ccc4ccc5ccccc5c4c3)c2)cc1 ; c1ccc(-c2cccc(N(c3ccc4c(ccc5ccccc54)c3)c3ccc4c(c3)oc3ccc5nc(-c6ccccc6)oc5c34)c2)cc1 ; c1ccc(-c2cccc(N(c3ccc4c(c3)oc3ccc5nc(-c6ccccc6)oc5c34)c3cc4ccccc4c4ccccc34)c2)cc1]

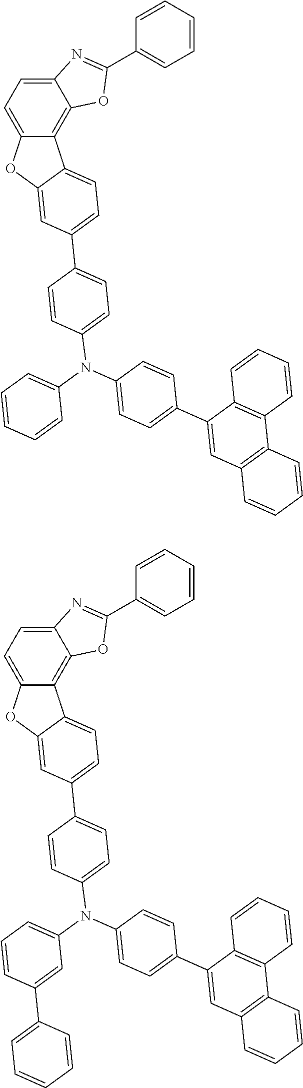 [SMILES: c1ccc(-c2nc3ccc4oc5cc(-c6ccc(N(c7ccccc7)c7ccc(-c8cc9ccccc9c9ccccc89)cc7)cc6)ccc5c4c3o2)cc1 ; c1ccc(-c2cccc(N(c3ccc(-c4ccc5c(c4)oc4ccc6nc(-c7ccccc7)oc6c45)cc3)c3ccc(-c4cc5ccccc5c5ccccc45)cc3)c2)cc1]

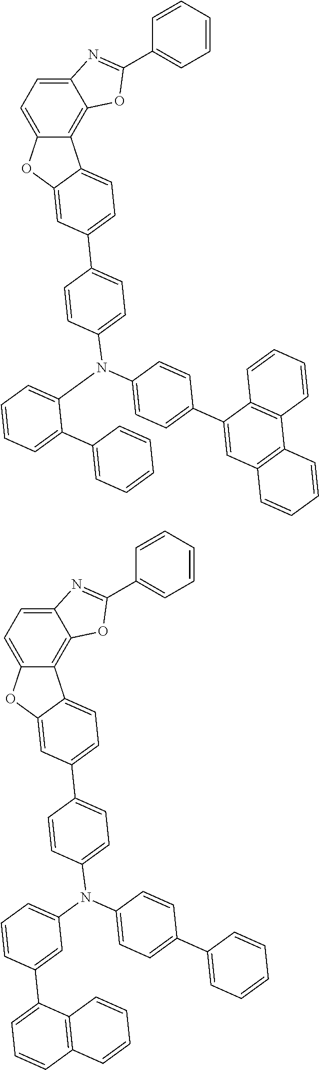 [SMILES: c1ccc(-c2nc3ccc4oc5cc(-c6ccc(N(c7ccc(-c8cc9ccccc9c9ccccc89)cc7)c7ccccc7-c7ccccc7)cc6)ccc5c4c3o2)cc1 ; c1ccc(-c2ccc(N(c3ccc(-c4ccc5c(c4)oc4ccc6nc(-c7ccccc7)oc6c45)cc3)c3cccc(-c4cccc5ccccc45)c3)cc2)cc1]

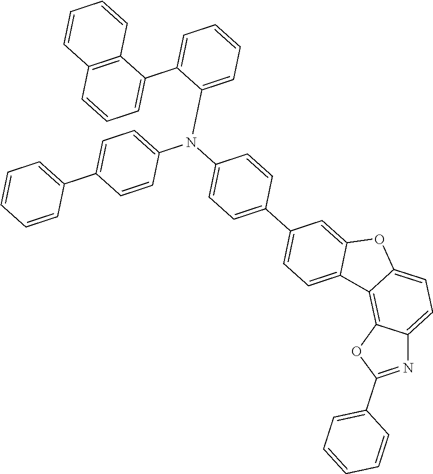 [SMILES: c1ccc(-c2ccc(N(c3ccc(-c4ccc5c(c4)oc4ccc6nc(-c7ccccc7)oc6c45)cc3)c3ccccc3-c3cccc4ccccc34)cc2)cc1]

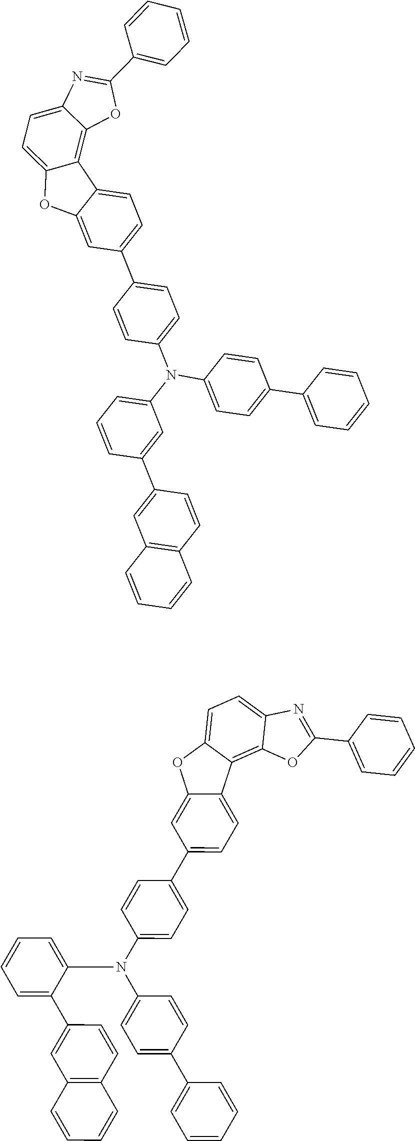 [SMILES: c1ccc(-c2ccc(N(c3ccc(-c4ccc5c(c4)oc4ccc6nc(-c7ccccc7)oc6c45)cc3)c3cccc(-c4ccc5ccccc5c4)c3)cc2)cc1 ; c1ccc(-c2ccc(N(c3ccc(-c4ccc5c(c4)oc4ccc6nc(-c7ccccc7)oc6c45)cc3)c3ccccc3-c3ccc4ccccc4c3)cc2)cc1]

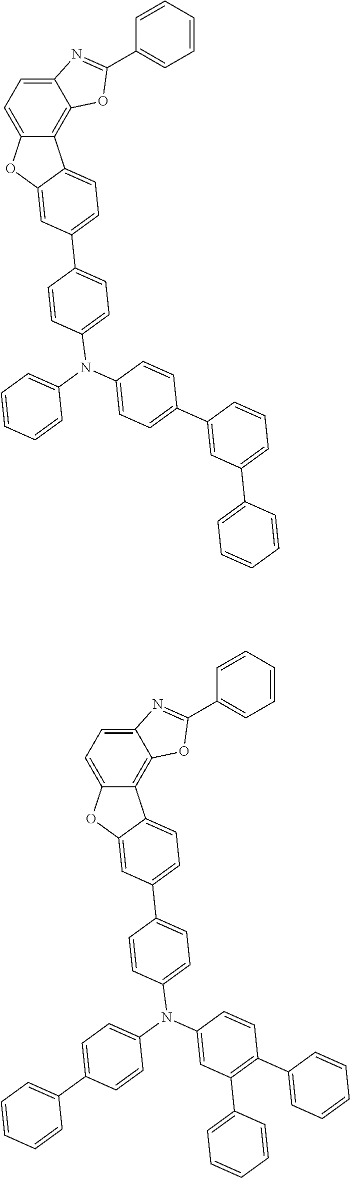 [SMILES: c1ccc(-c2cccc(-c3ccc(N(c4ccccc4)c4ccc(-c5ccc6c(c5)oc5ccc7nc(-c8ccccc8)oc7c56)cc4)cc3)c2)cc1 ; c1ccc(-c2ccc(N(c3ccc(-c4ccc5c(c4)oc4ccc6nc(-c7ccccc7)oc6c45)cc3)c3ccc(-c4ccccc4)c(-c4ccccc4)c3)cc2)cc1]

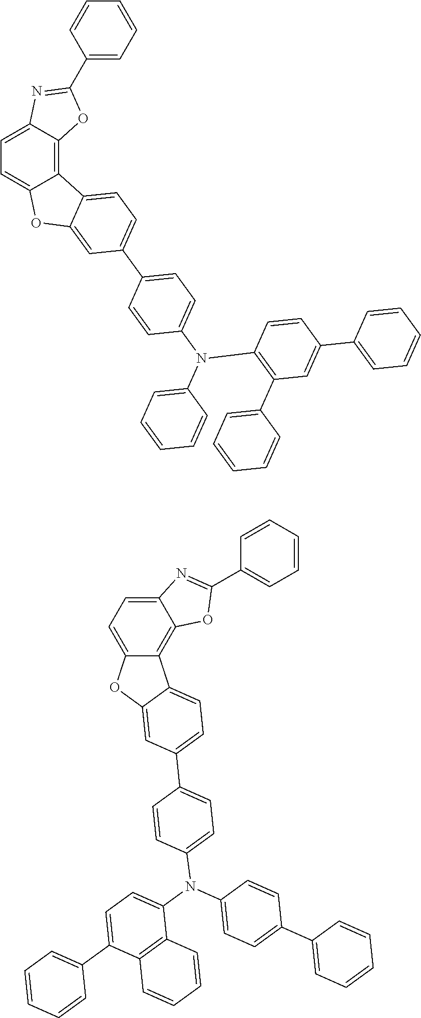 [SMILES: c1ccc(-c2ccc(N(c3ccccc3)c3ccc(-c4ccc5c(c4)oc4ccc6nc(-c7ccccc7)oc6c45)cc3)c(-c3ccccc3)c2)cc1 ; c1ccc(-c2ccc(N(c3ccc(-c4ccc5c(c4)oc4ccc6nc(-c7ccccc7)oc6c45)cc3)c3ccc(-c4ccccc4)c4ccccc34)cc2)cc1]

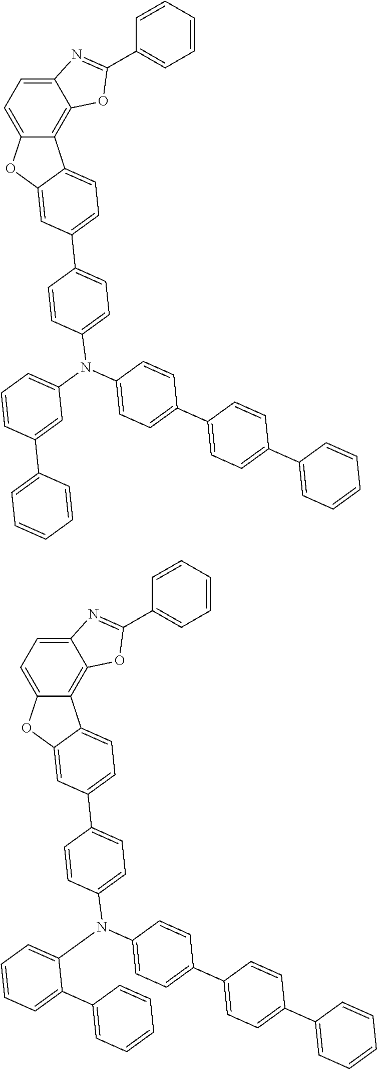 [SMILES: c1ccc(-c2ccc(-c3ccc(N(c4ccc(-c5ccc6c(c5)oc5ccc7nc(-c8ccccc8)oc7c56)cc4)c4cccc(-c5ccccc5)c4)cc3)cc2)cc1 ; c1ccc(-c2ccc(-c3ccc(N(c4ccc(-c5ccc6c(c5)oc5ccc7nc(-c8ccccc8)oc7c56)cc4)c4ccccc4-c4ccccc4)cc3)cc2)cc1]

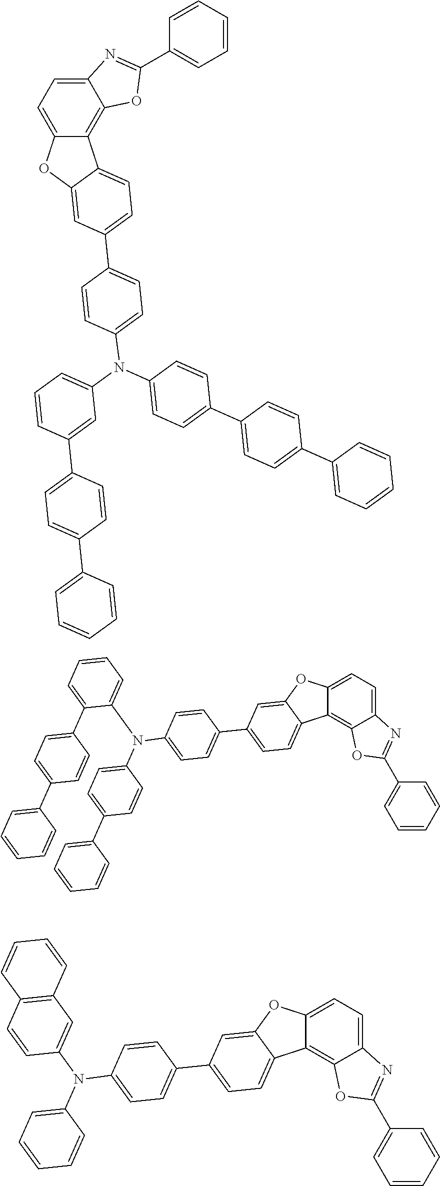

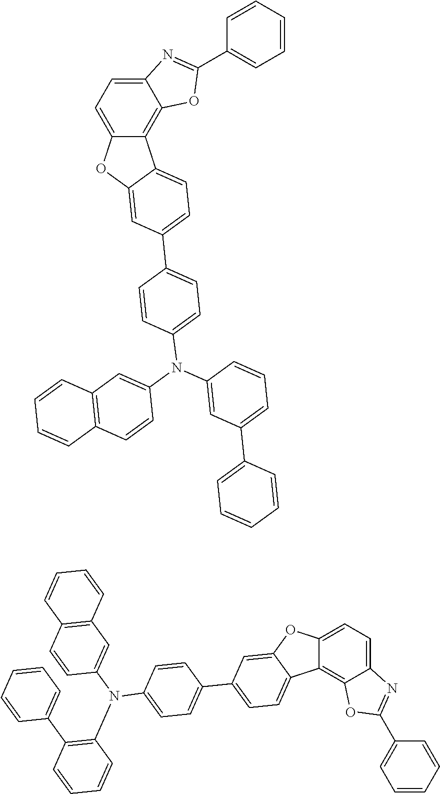 [SMILES: c1ccc(-c2cccc(N(c3ccc(-c4ccc5c(c4)oc4ccc6nc(-c7ccccc7)oc6c45)cc3)c3ccc4ccccc4c3)c2)cc1 ; c1ccc(-c2nc3ccc4oc5cc(-c6ccc(N(c7ccc8ccccc8c7)c7ccccc7-c7ccccc7)cc6)ccc5c4c3o2)cc1]

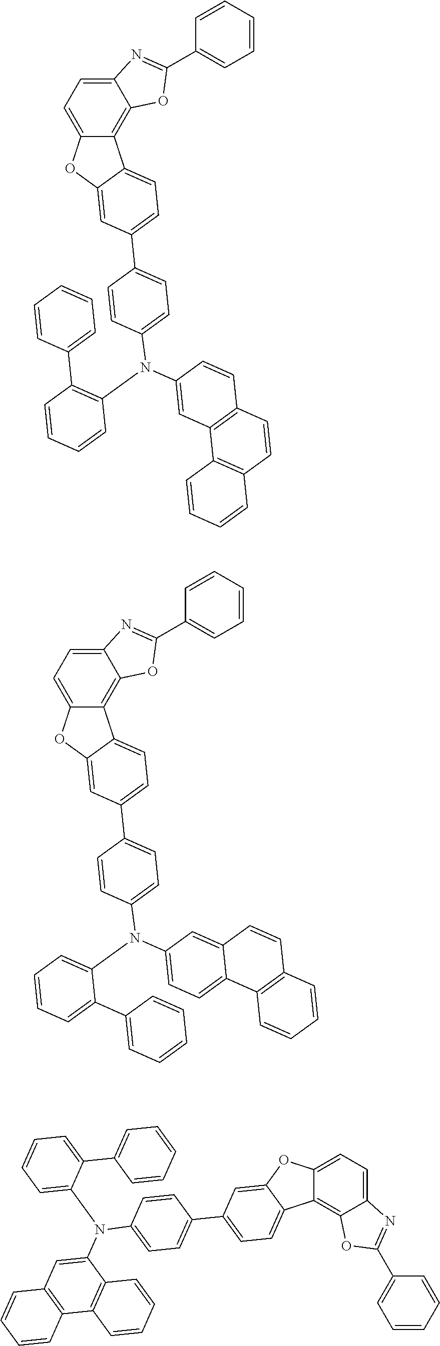

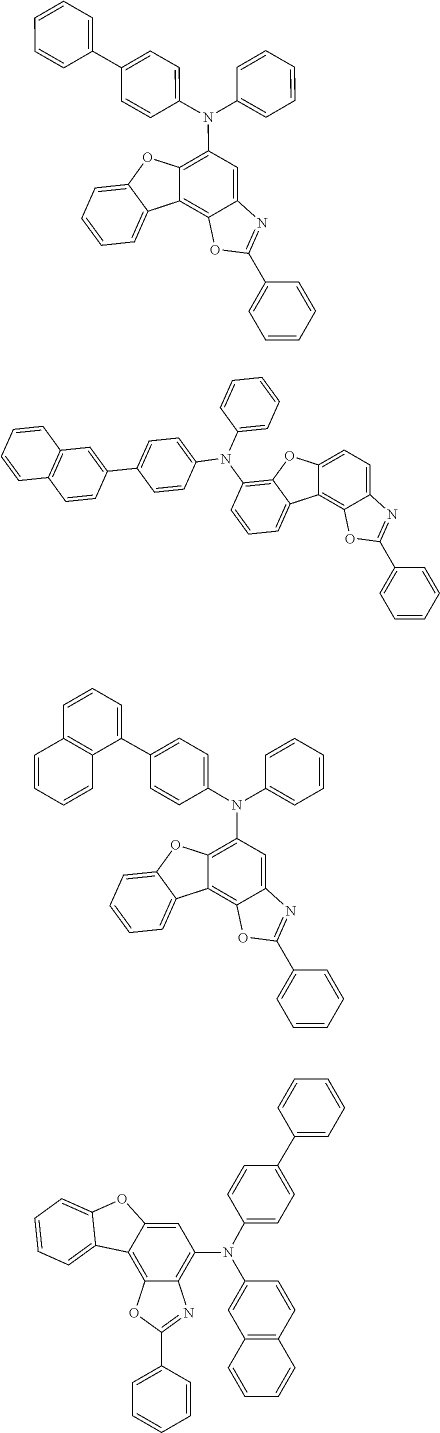

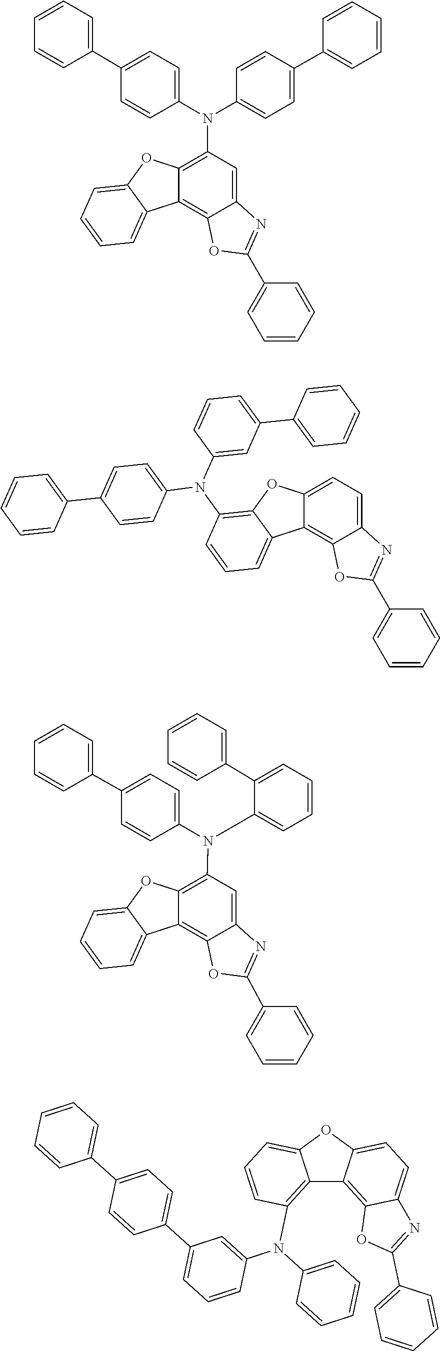 [SMILES: c1ccc(-c2ccc(N(c3ccc(-c4ccccc4)cc3)c3cc4nc(-c5ccccc5)oc4c4c3oc3ccccc34)cc2)cc1 ; c1ccc(-c2ccc(N(c3cccc(-c4ccccc4)c3)c3cccc4c3oc3ccc5nc(-c6ccccc6)oc5c34)cc2)cc1 ; c1ccc(-c2ccc(N(c3ccccc3-c3ccccc3)c3cc4nc(-c5ccccc5)oc4c4c3oc3ccccc34)cc2)cc1 ; c1ccc(-c2ccc(-c3cccc(N(c4ccccc4)c4cccc5oc6ccc7nc(-c8ccccc8)oc7c6c45)c3)cc2)cc1]

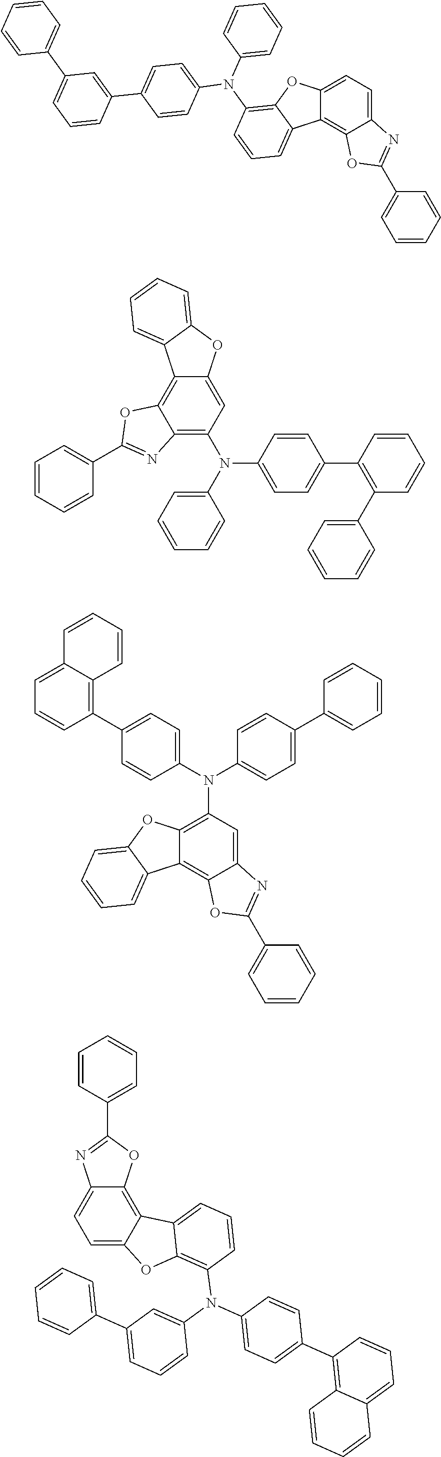 [SMILES: c1ccc(-c2cccc(-c3ccc(N(c4ccccc4)c4cccc5c4oc4ccc6nc(-c7ccccc7)oc6c45)cc3)c2)cc1 ; c1ccc(-c2nc3c(N(c4ccccc4)c4ccc(-c5ccccc5-c5ccccc5)cc4)cc4oc5ccccc5c4c3o2)cc1 ; c1ccc(-c2ccc(N(c3ccc(-c4cccc5ccccc45)cc3)c3cc4nc(-c5ccccc5)oc4c4c3oc3ccccc34)cc2)cc1 ; c1ccc(-c2cccc(N(c3ccc(-c4cccc5ccccc45)cc3)c3cccc4c3oc3ccc5nc(-c6ccccc6)oc5c34)c2)cc1]

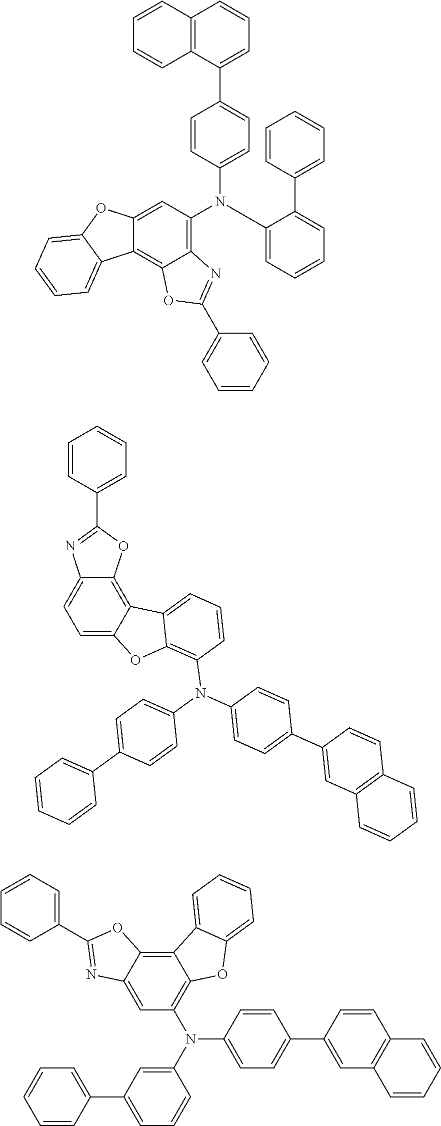 [SMILES: c1ccc(-c2nc3c(N(c4ccc(-c5cccc6ccccc56)cc4)c4ccccc4-c4ccccc4)cc4oc5ccccc5c4c3o2)cc1 ; c1ccc(-c2ccc(N(c3ccc(-c4ccc5ccccc5c4)cc3)c3cccc4c3oc3ccc5nc(-c6ccccc6)oc5c34)cc2)cc1 ; c1ccc(-c2cccc(N(c3ccc(-c4ccc5ccccc5c4)cc3)c3cc4nc(-c5ccccc5)oc4c4c3oc3ccccc34)c2)cc1]

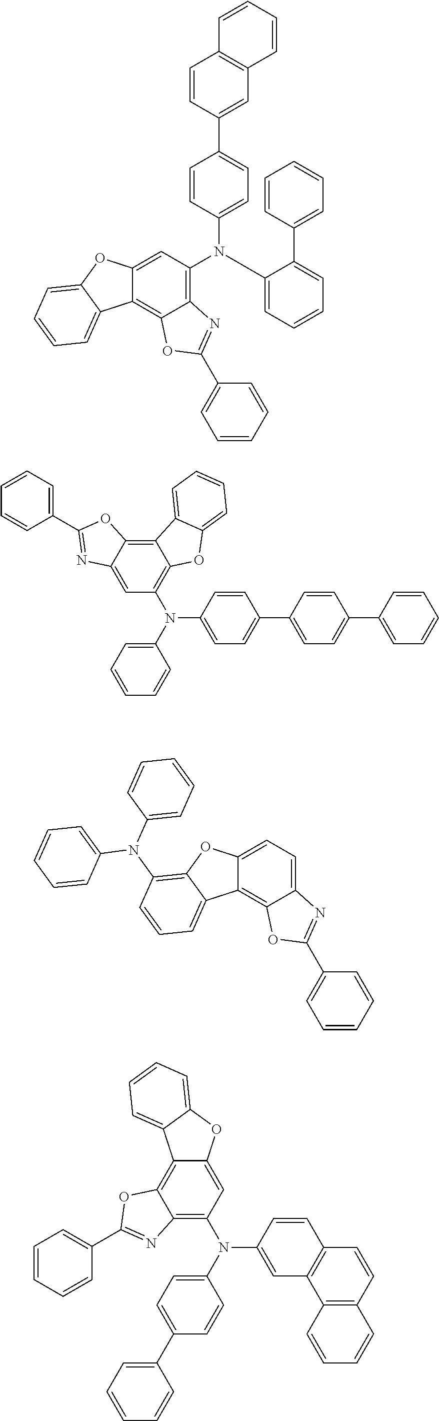

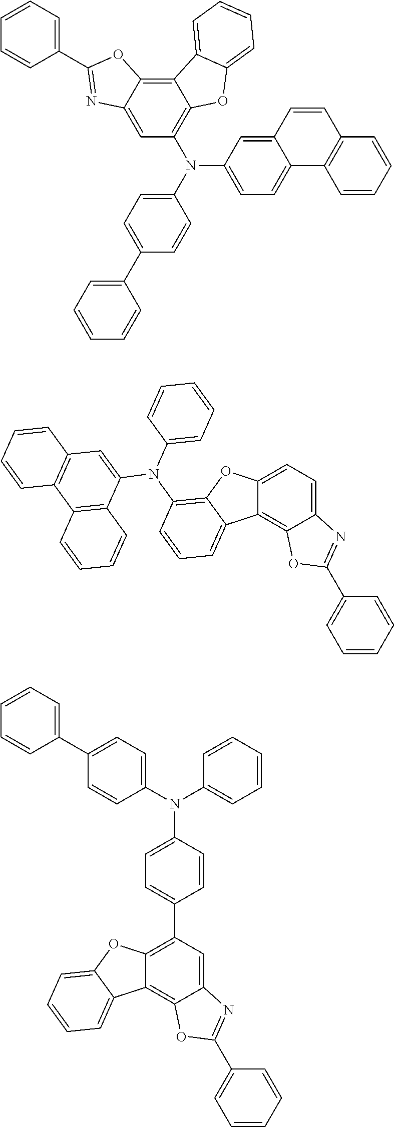 [SMILES: c1ccc(-c2ccc(N(c3ccc4c(ccc5ccccc54)c3)c3cc4nc(-c5ccccc5)oc4c4c3oc3ccccc34)cc2)cc1 ; c1ccc(-c2nc3ccc4oc5c(N(c6ccccc6)c6cc7ccccc7c7ccccc67)cccc5c4c3o2)cc1 ; c1ccc(-c2ccc(N(c3ccccc3)c3ccc(-c4cc5nc(-c6ccccc6)oc5c5c4oc4ccccc45)cc3)cc2)cc1]

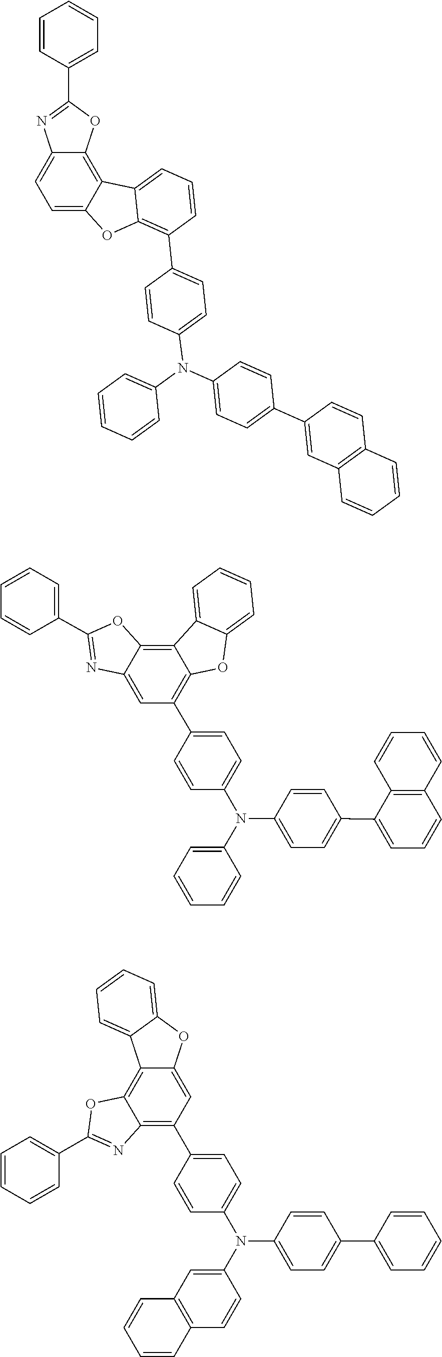 [SMILES: c1ccc(-c2nc3ccc4oc5c(-c6ccc(N(c7ccccc7)c7ccc(-c8ccc9ccccc9c8)cc7)cc6)cccc5c4c3o2)cc1 ; c1ccc(-c2nc3cc(-c4ccc(N(c5ccccc5)c5ccc(-c6cccc7ccccc67)cc5)cc4)c4oc5ccccc5c4c3o2)cc1 ; c1ccc(-c2ccc(N(c3ccc(-c4cc5oc6ccccc6c5c5oc(-c6ccccc6)nc45)cc3)c3ccc4ccccc4c3)cc2)cc1]

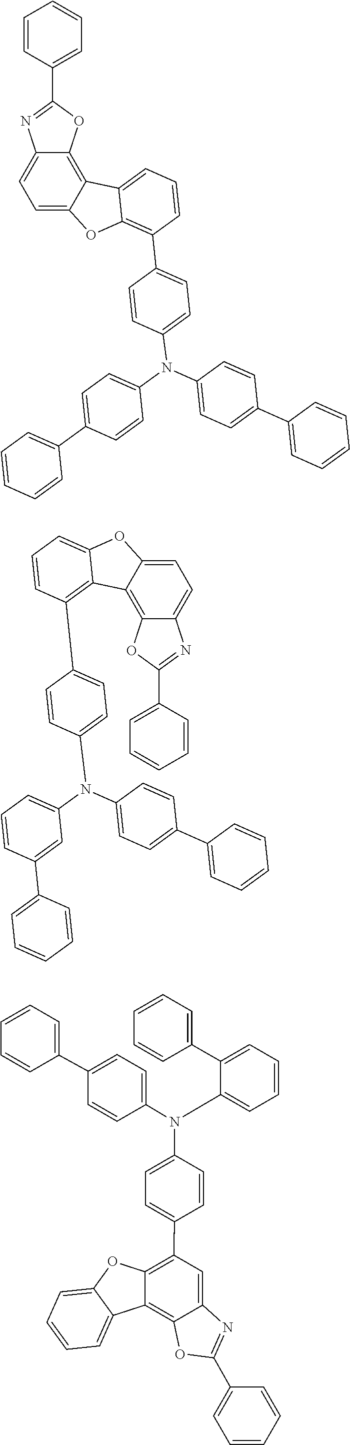

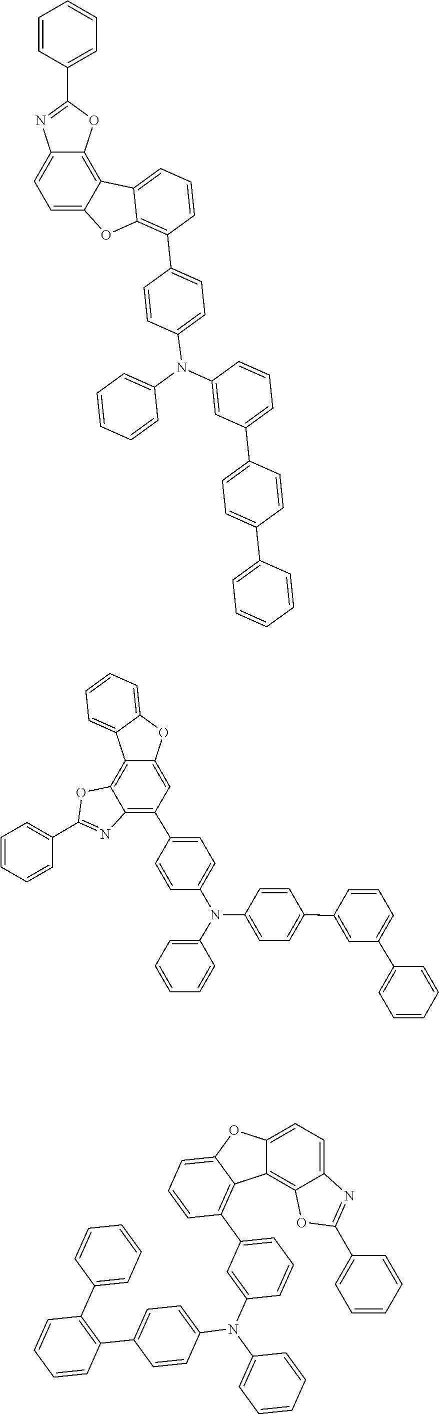 [SMILES: c1ccc(-c2ccc(-c3cccc(N(c4ccccc4)c4ccc(-c5cccc6c5oc5ccc7nc(-c8ccccc8)oc7c56)cc4)c3)cc2)cc1 ; c1ccc(-c2cccc(-c3ccc(N(c4ccccc4)c4ccc(-c5cc6oc7ccccc7c6c6oc(-c7ccccc7)nc56)cc4)cc3)c2)cc1 ; c1ccc(-c2nc3ccc4oc5cccc(-c6cccc(N(c7ccccc7)c7ccc(-c8ccccc8-c8ccccc8)cc7)c6)c5c4c3o2)cc1]

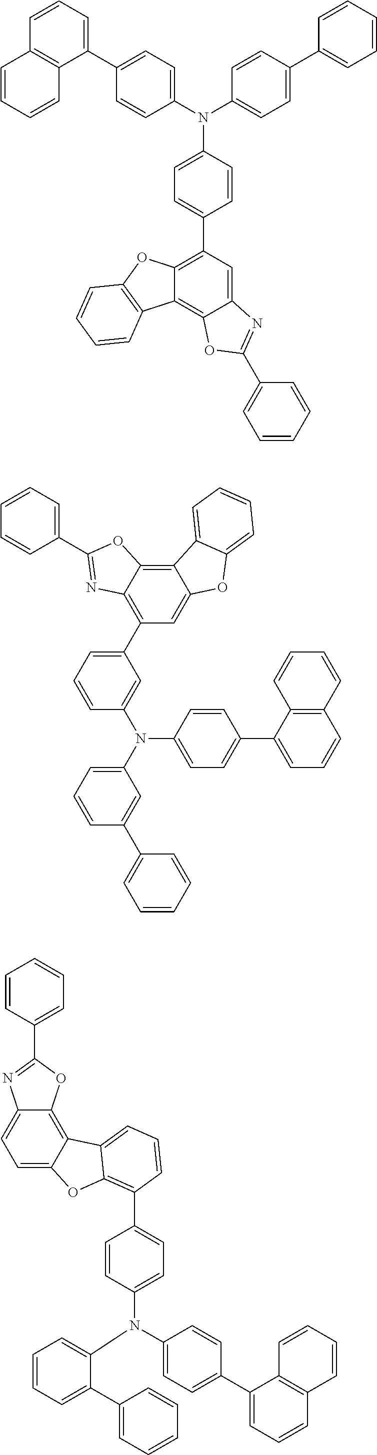 [SMILES: c1ccc(-c2ccc(N(c3ccc(-c4cccc5ccccc45)cc3)c3ccc(-c4cc5nc(-c6ccccc6)oc5c5c4oc4ccccc45)cc3)cc2)cc1 ; c1ccc(-c2cccc(N(c3ccc(-c4cccc5ccccc45)cc3)c3cccc(-c4cc5oc6ccccc6c5c5oc(-c6ccccc6)nc45)c3)c2)cc1 ; c1ccc(-c2nc3ccc4oc5c(-c6ccc(N(c7ccc(-c8cccc9ccccc89)cc7)c7ccccc7-c7ccccc7)cc6)cccc5c4c3o2)cc1]

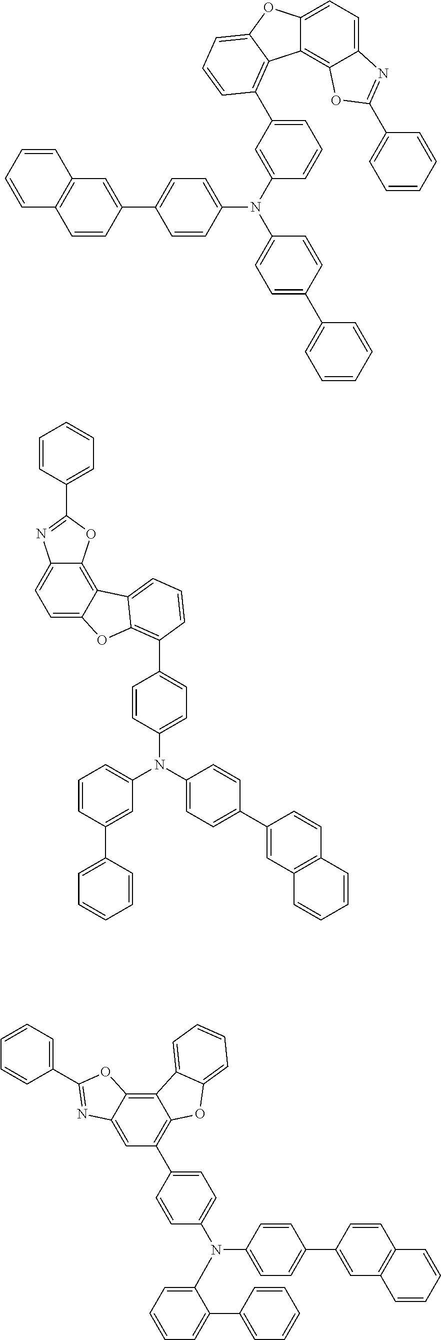 [SMILES: c1ccc(-c2ccc(N(c3ccc(-c4ccc5ccccc5c4)cc3)c3cccc(-c4cccc5oc6ccc7nc(-c8ccccc8)oc7c6c45)c3)cc2)cc1 ; c1ccc(-c2cccc(N(c3ccc(-c4ccc5ccccc5c4)cc3)c3ccc(-c4cccc5c4oc4ccc6nc(-c7ccccc7)oc6c45)cc3)c2)cc1 ; c1ccc(-c2nc3cc(-c4ccc(N(c5ccc(-c6ccc7ccccc7c6)cc5)c5ccccc5-c5ccccc5)cc4)c4oc5ccccc5c4c3o2)cc1]

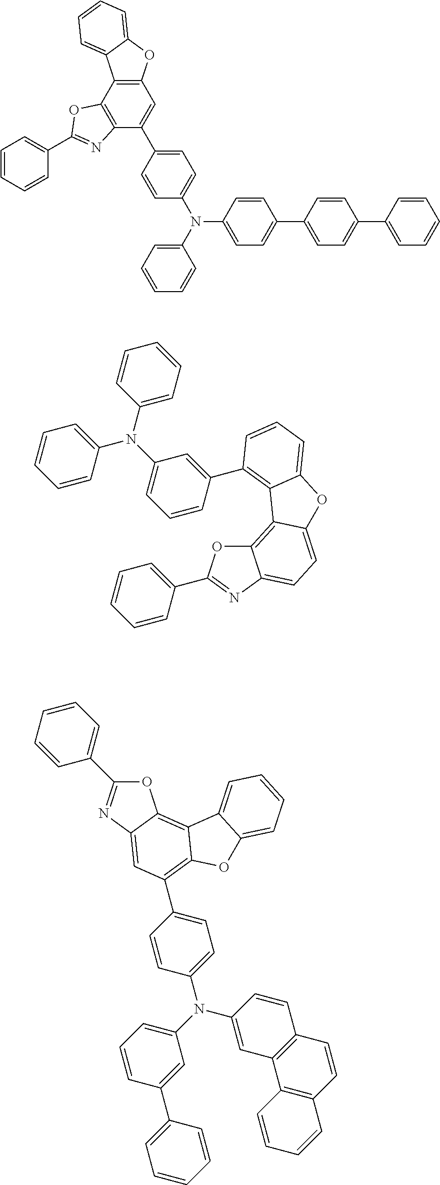 [SMILES: c1ccc(-c2ccc(-c3ccc(N(c4ccccc4)c4ccc(-c5cc6oc7ccccc7c6c6oc(-c7ccccc7)nc56)cc4)cc3)cc2)cc1 ; c1ccc(-c2nc3ccc4oc5cccc(-c6cccc(N(c7ccccc7)c7ccccc7)c6)c5c4c3o2)cc1 ; c1ccc(-c2cccc(N(c3ccc(-c4cc5nc(-c6ccccc6)oc5c5c4oc4ccccc45)cc3)c3ccc4ccc5ccccc5c4c3)c2)cc1]

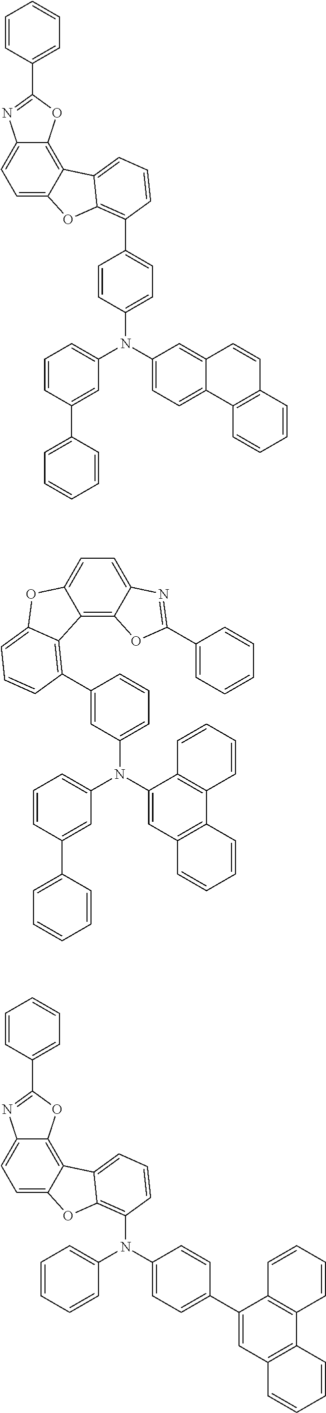 [SMILES: c1ccc(-c2cccc(N(c3ccc(-c4cccc5c4oc4ccc6nc(-c7ccccc7)oc6c45)cc3)c3ccc4c(ccc5ccccc54)c3)c2)cc1 ; c1ccc(-c2cccc(N(c3cccc(-c4cccc5oc6ccc7nc(-c8ccccc8)oc7c6c45)c3)c3cc4ccccc4c4ccccc34)c2)cc1 ; c1ccc(-c2nc3ccc4oc5c(N(c6ccccc6)c6ccc(-c7cc8ccccc8c8ccccc78)cc6)cccc5c4c3o2)cc1]

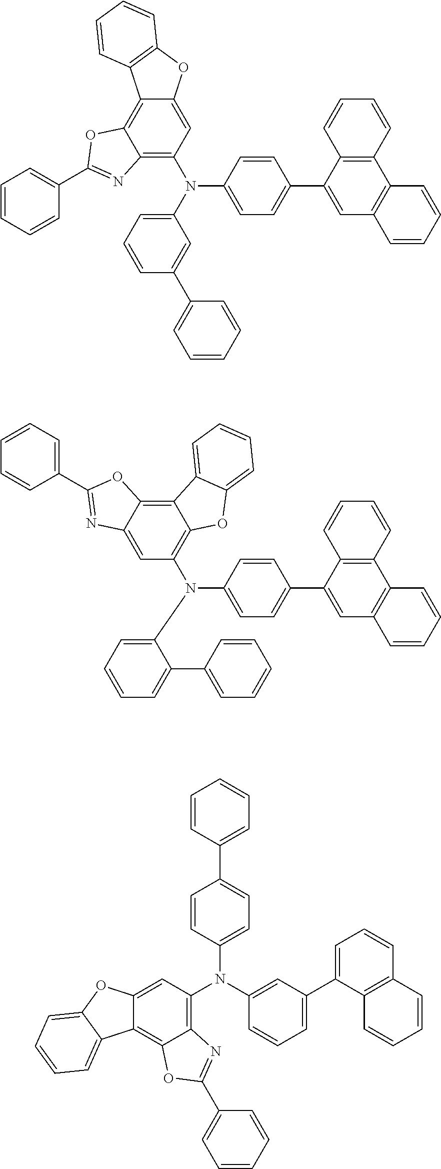 [SMILES: c1ccc(-c2cccc(N(c3ccc(-c4cc5ccccc5c5ccccc45)cc3)c3cc4oc5ccccc5c4c4oc(-c5ccccc5)nc34)c2)cc1 ; c1ccc(-c2nc3cc(N(c4ccc(-c5cc6ccccc6c6ccccc56)cc4)c4ccccc4-c4ccccc4)c4oc5ccccc5c4c3o2)cc1 ; c1ccc(-c2ccc(N(c3cccc(-c4cccc5ccccc45)c3)c3cc4oc5ccccc5c4c4oc(-c5ccccc5)nc34)cc2)cc1]

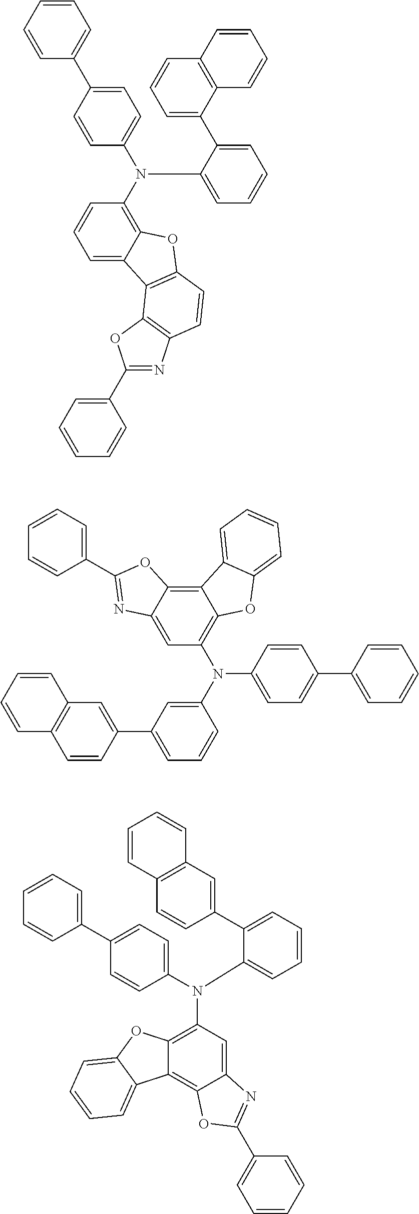 [SMILES: c1ccc(-c2ccc(N(c3ccccc3-c3cccc4ccccc34)c3cccc4c3oc3ccc5nc(-c6ccccc6)oc5c34)cc2)cc1 ; c1ccc(-c2ccc(N(c3cccc(-c4ccc5ccccc5c4)c3)c3cc4nc(-c5ccccc5)oc4c4c3oc3ccccc34)cc2)cc1 ; c1ccc(-c2ccc(N(c3ccccc3-c3ccc4ccccc4c3)c3cc4nc(-c5ccccc5)oc4c4c3oc3ccccc34)cc2)cc1]

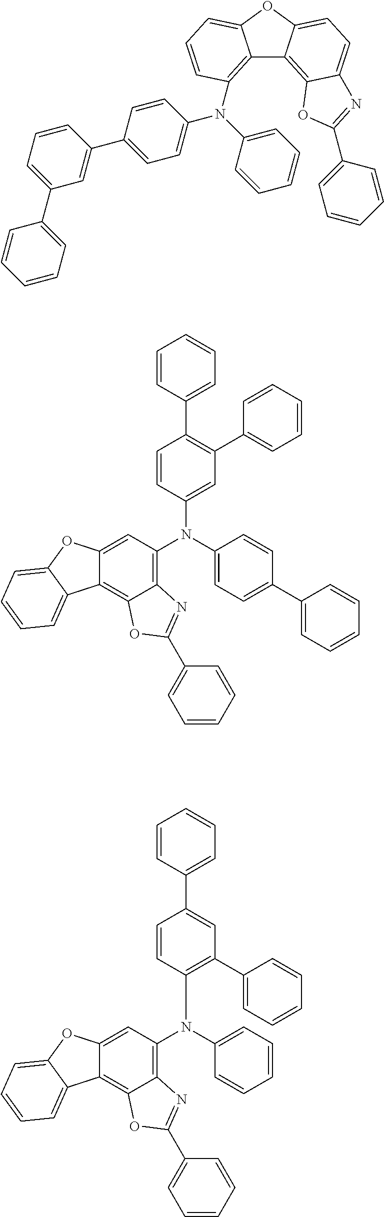 [SMILES: c1ccc(-c2cccc(-c3ccc(N(c4ccccc4)c4cccc5oc6ccc7nc(-c8ccccc8)oc7c6c45)cc3)c2)cc1 ; c1ccc(-c2ccc(N(c3ccc(-c4ccccc4)c(-c4ccccc4)c3)c3cc4oc5ccccc5c4c4oc(-c5ccccc5)nc34)cc2)cc1 ; c1ccc(-c2ccc(N(c3ccccc3)c3cc4oc5ccccc5c4c4oc(-c5ccccc5)nc34)c(-c3ccccc3)c2)cc1]

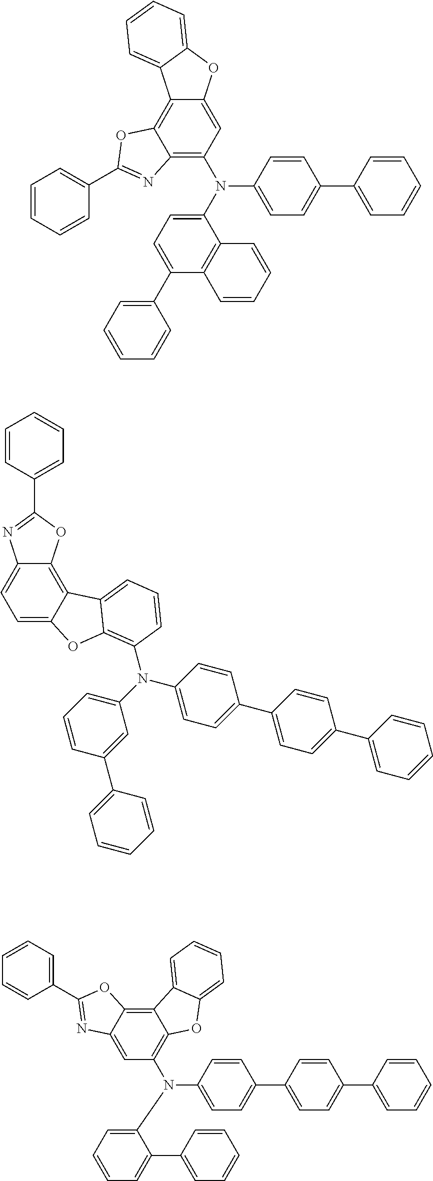 [SMILES: c1ccc(-c2ccc(N(c3ccc(-c4ccccc4)c4ccccc34)c3cc4oc5ccccc5c4c4oc(-c5ccccc5)nc34)cc2)cc1 ; c1ccc(-c2ccc(-c3ccc(N(c4cccc(-c5ccccc5)c4)c4cccc5c4oc4ccc6nc(-c7ccccc7)oc6c45)cc3)cc2)cc1 ; c1ccc(-c2ccc(-c3ccc(N(c4ccccc4-c4ccccc4)c4cc5nc(-c6ccccc6)oc5c5c4oc4ccccc45)cc3)cc2)cc1]

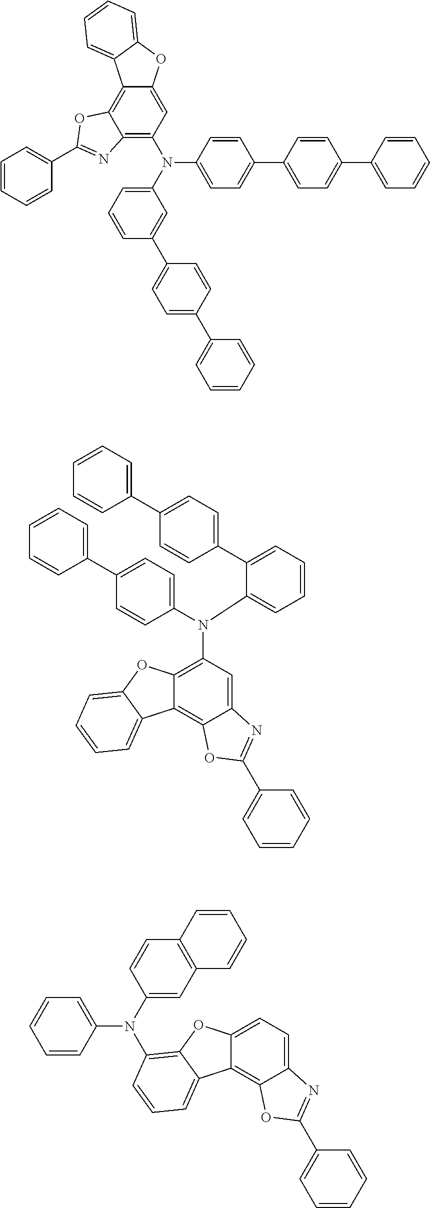 [SMILES: c1ccc(-c2ccc(-c3ccc(N(c4cccc(-c5ccc(-c6ccccc6)cc5)c4)c4cc5oc6ccccc6c5c5oc(-c6ccccc6)nc45)cc3)cc2)cc1 ; c1ccc(-c2ccc(-c3ccccc3N(c3ccc(-c4ccccc4)cc3)c3cc4nc(-c5ccccc5)oc4c4c3oc3ccccc34)cc2)cc1 ; c1ccc(-c2nc3ccc4oc5c(N(c6ccccc6)c6ccc7ccccc7c6)cccc5c4c3o2)cc1]

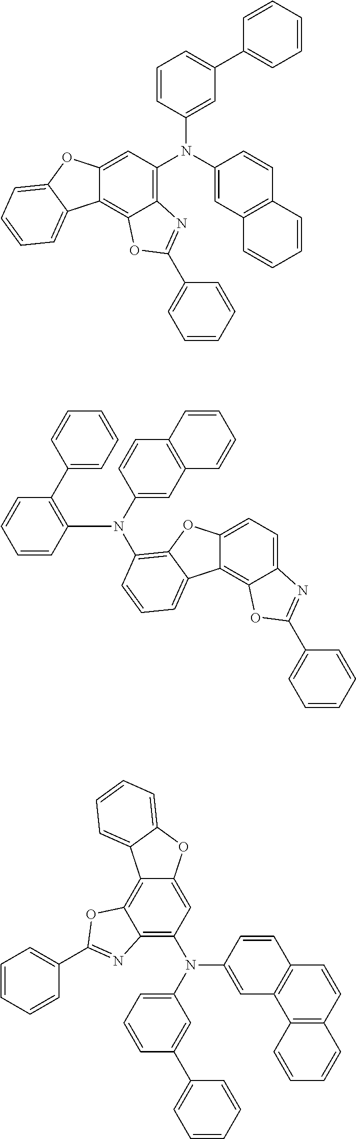 [SMILES: c1ccc(-c2cccc(N(c3ccc4ccccc4c3)c3cc4oc5ccccc5c4c4oc(-c5ccccc5)nc34)c2)cc1 ; c1ccc(-c2nc3ccc4oc5c(N(c6ccc7ccccc7c6)c6ccccc6-c6ccccc6)cccc5c4c3o2)cc1 ; c1ccc(-c2cccc(N(c3ccc4ccc5ccccc5c4c3)c3cc4oc5ccccc5c4c4oc(-c5ccccc5)nc34)c2)cc1]

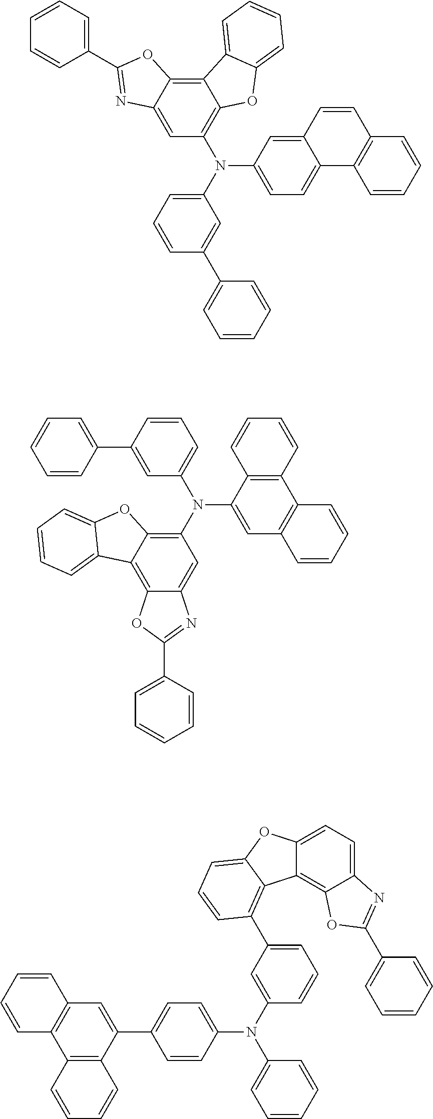 [SMILES: c1ccc(-c2cccc(N(c3ccc4c(ccc5ccccc54)c3)c3cc4nc(-c5ccccc5)oc4c4c3oc3ccccc34)c2)cc1 ; c1ccc(-c2cccc(N(c3cc4ccccc4c4ccccc34)c3cc4nc(-c5ccccc5)oc4c4c3oc3ccccc34)c2)cc1 ; c1ccc(-c2nc3ccc4oc5cccc(-c6cccc(N(c7ccccc7)c7ccc(-c8cc9ccccc9c9ccccc89)cc7)c6)c5c4c3o2)cc1]

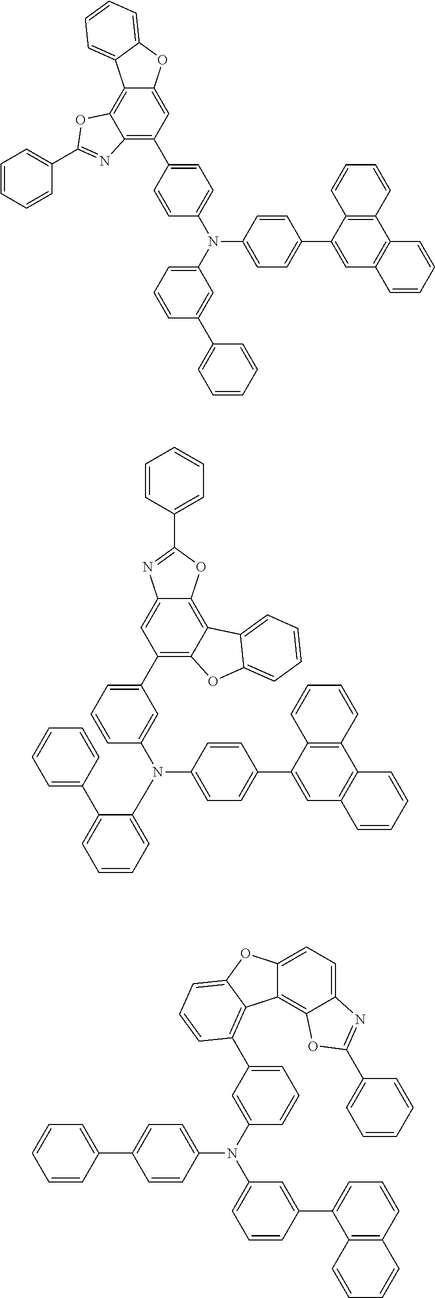 [SMILES: c1ccc(-c2cccc(N(c3ccc(-c4cc5ccccc5c5ccccc45)cc3)c3ccc(-c4cc5oc6ccccc6c5c5oc(-c6ccccc6)nc45)cc3)c2)cc1 ; c1ccc(-c2nc3cc(-c4cccc(N(c5ccc(-c6cc7ccccc7c7ccccc67)cc5)c5ccccc5-c5ccccc5)c4)c4oc5ccccc5c4c3o2)cc1 ; c1ccc(-c2ccc(N(c3cccc(-c4cccc5ccccc45)c3)c3cccc(-c4cccc5oc6ccc7nc(-c8ccccc8)oc7c6c45)c3)cc2)cc1]

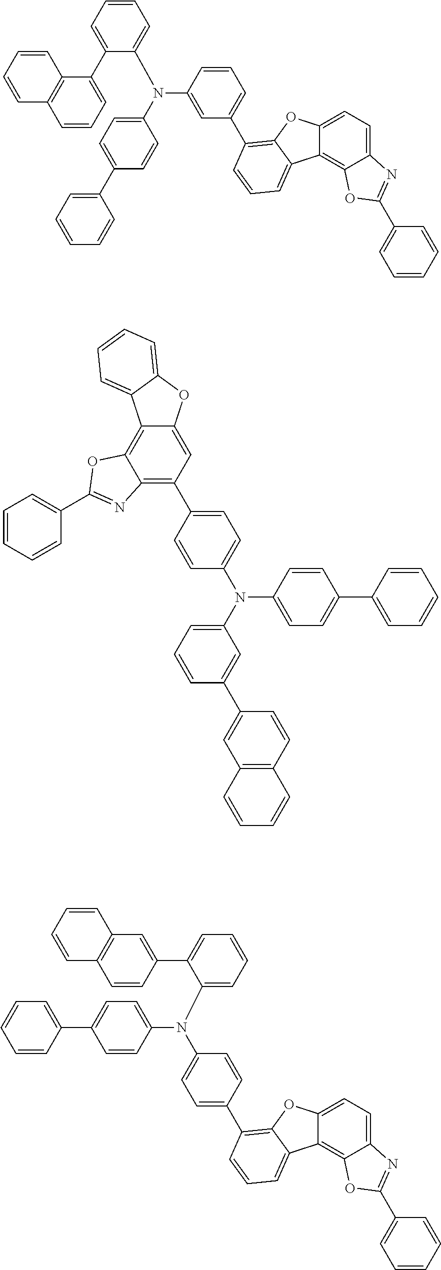 [SMILES: c1ccc(-c2ccc(N(c3cccc(-c4cccc5c4oc4ccc6nc(-c7ccccc7)oc6c45)c3)c3ccccc3-c3cccc4ccccc34)cc2)cc1 ; c1ccc(-c2ccc(N(c3ccc(-c4cc5oc6ccccc6c5c5oc(-c6ccccc6)nc45)cc3)c3cccc(-c4ccc5ccccc5c4)c3)cc2)cc1 ; c1ccc(-c2ccc(N(c3ccc(-c4cccc5c4oc4ccc6nc(-c7ccccc7)oc6c45)cc3)c3ccccc3-c3ccc4ccccc4c3)cc2)cc1]

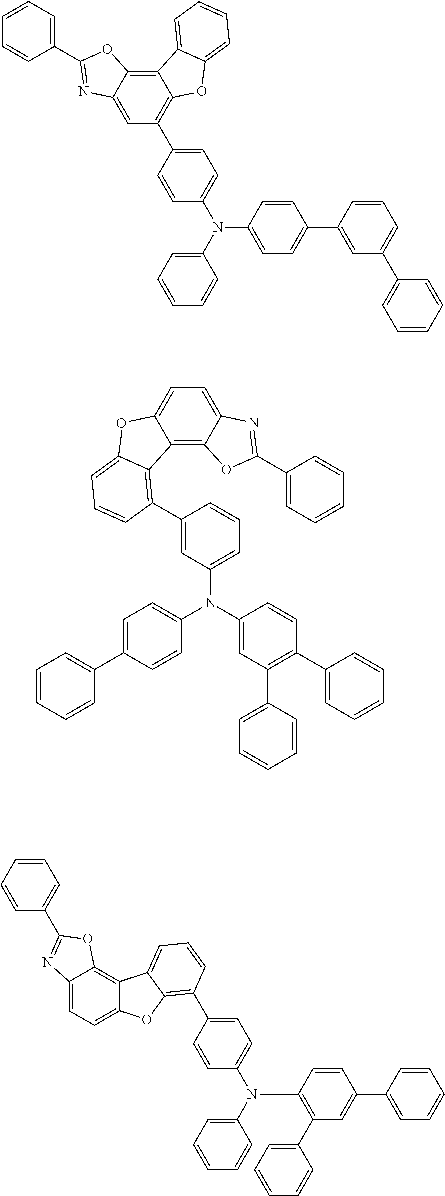 [SMILES: c1ccc(-c2cccc(-c3ccc(N(c4ccccc4)c4ccc(-c5cc6nc(-c7ccccc7)oc6c6c5oc5ccccc56)cc4)cc3)c2)cc1 ; c1ccc(-c2ccc(N(c3cccc(-c4cccc5oc6ccc7nc(-c8ccccc8)oc7c6c45)c3)c3ccc(-c4ccccc4)c(-c4ccccc4)c3)cc2)cc1 ; c1ccc(-c2ccc(N(c3ccccc3)c3ccc(-c4cccc5c4oc4ccc6nc(-c7ccccc7)oc6c45)cc3)c(-c3ccccc3)c2)cc1]

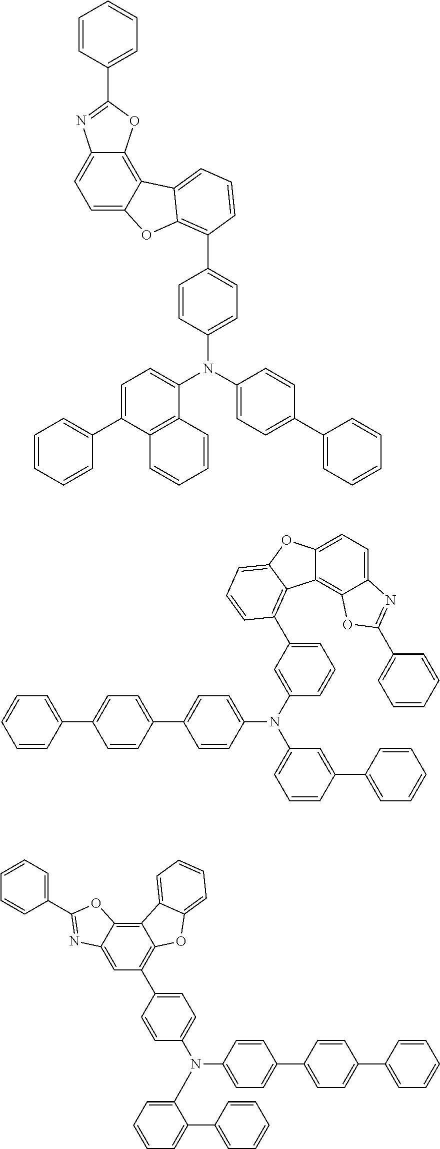 [SMILES: c1ccc(-c2ccc(N(c3ccc(-c4cccc5c4oc4ccc6nc(-c7ccccc7)oc6c45)cc3)c3ccc(-c4ccccc4)c4ccccc34)cc2)cc1 ; c1ccc(-c2ccc(-c3ccc(N(c4cccc(-c5ccccc5)c4)c4cccc(-c5cccc6oc7ccc8nc(-c9ccccc9)oc8c7c56)c4)cc3)cc2)cc1 ; c1ccc(-c2ccc(-c3ccc(N(c4ccc(-c5cc6nc(-c7ccccc7)oc6c6c5oc5ccccc56)cc4)c4ccccc4-c4ccccc4)cc3)cc2)cc1]

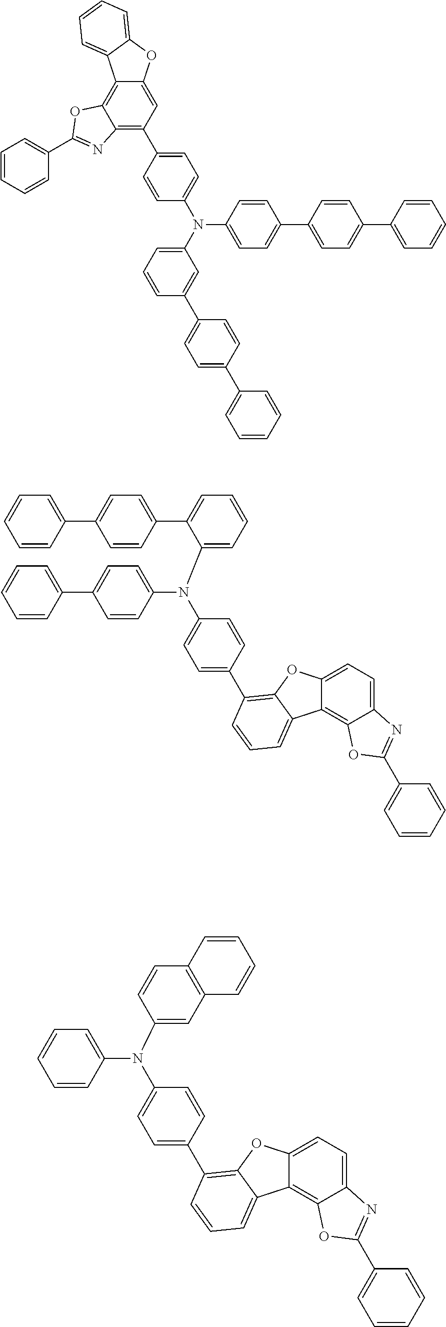 [SMILES: c1ccc(-c2ccc(-c3ccc(N(c4ccc(-c5cc6oc7ccccc7c6c6oc(-c7ccccc7)nc56)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4cccc5c4oc4ccc6nc(-c7ccccc7)oc6c45)cc3)cc2)cc1 ; c1ccc(-c2nc3ccc4oc5c(-c6ccc(N(c7ccccc7)c7ccc8ccccc8c7)cc6)cccc5c4c3o2)cc1]

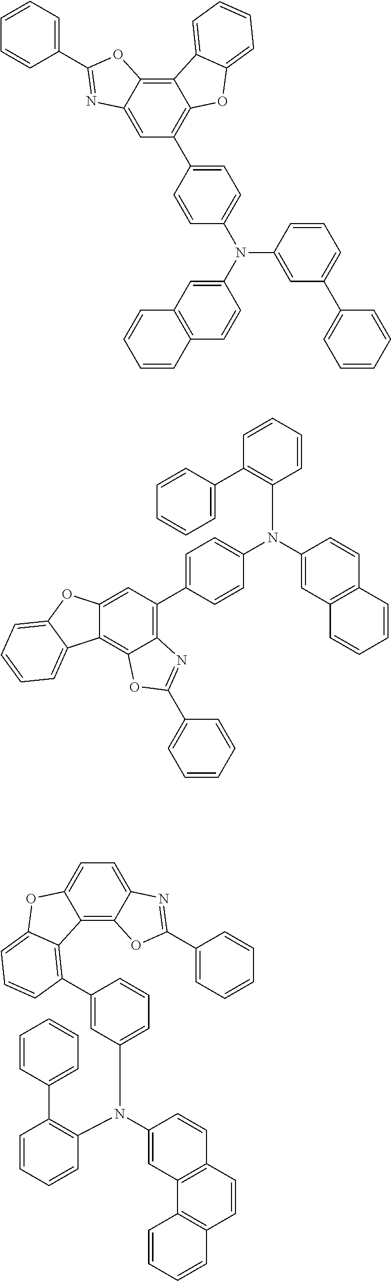 [SMILES: c1ccc(-c2cccc(N(c3ccc(-c4cc5nc(-c6ccccc6)oc5c5c4oc4ccccc45)cc3)c3ccc4ccccc4c3)c2)cc1 ; c1ccc(-c2nc3c(-c4ccc(N(c5ccc6ccccc6c5)c5ccccc5-c5ccccc5)cc4)cc4oc5ccccc5c4c3o2)cc1 ; c1ccc(-c2nc3ccc4oc5cccc(-c6cccc(N(c7ccc8ccc9ccccc9c8c7)c7ccccc7-c7ccccc7)c6)c5c4c3o2)cc1]

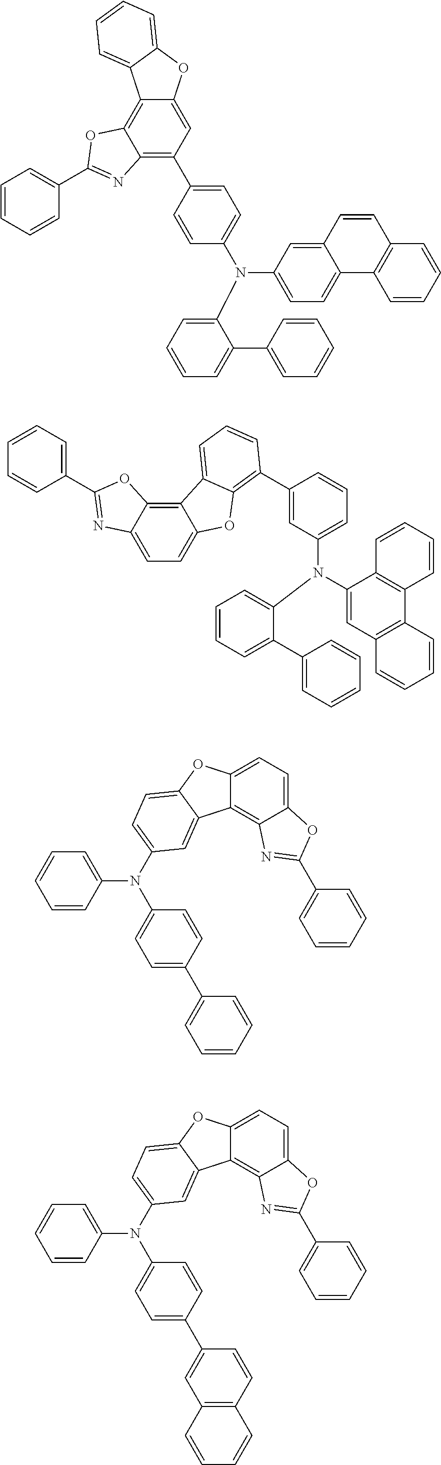 [SMILES: c1ccc(-c2nc3c(-c4ccc(N(c5ccc6c(ccc7ccccc76)c5)c5ccccc5-c5ccccc5)cc4)cc4oc5ccccc5c4c3o2)cc1 ; c1ccc(-c2nc3ccc4oc5c(-c6cccc(N(c7ccccc7-c7ccccc7)c7cc8ccccc8c8ccccc78)c6)cccc5c4c3o2)cc1 ; c1ccc(-c2ccc(N(c3ccccc3)c3ccc4oc5ccc6oc(-c7ccccc7)nc6c5c4c3)cc2)cc1 ; c1ccc(-c2nc3c(ccc4oc5ccc(N(c6ccccc6)c6ccc(-c7ccc8ccccc8c7)cc6)cc5c43)o2)cc1]

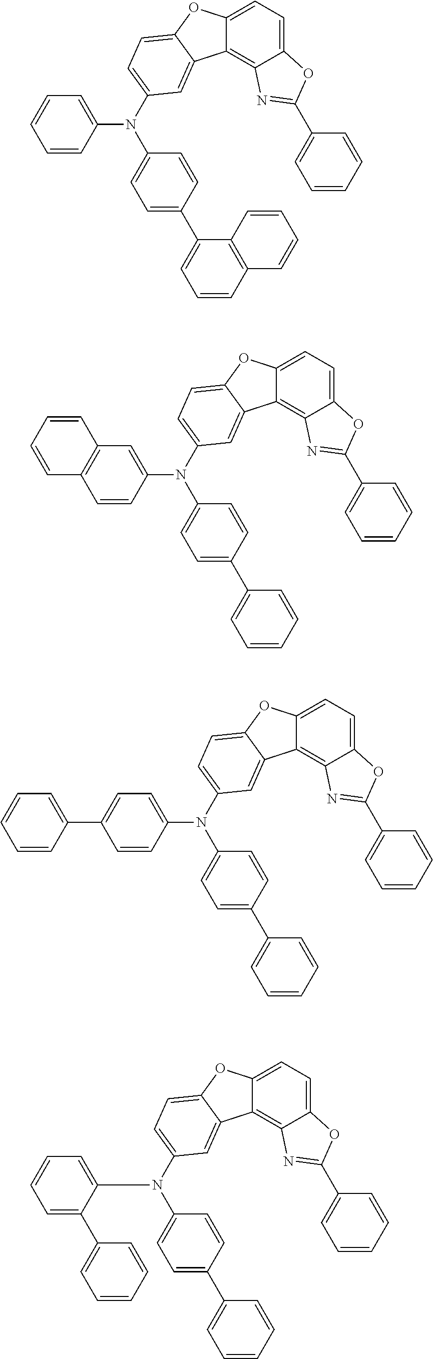 [SMILES: c1ccc(-c2nc3c(ccc4oc5ccc(N(c6ccccc6)c6ccc(-c7cccc8ccccc78)cc6)cc5c43)o2)cc1 ; c1ccc(-c2ccc(N(c3ccc4ccccc4c3)c3ccc4oc5ccc6oc(-c7ccccc7)nc6c5c4c3)cc2)cc1 ; c1ccc(-c2ccc(N(c3ccc(-c4ccccc4)cc3)c3ccc4oc5ccc6oc(-c7ccccc7)nc6c5c4c3)cc2)cc1 ; c1ccc(-c2ccc(N(c3ccc4oc5ccc6oc(-c7ccccc7)nc6c5c4c3)c3ccccc3-c3ccccc3)cc2)cc1]

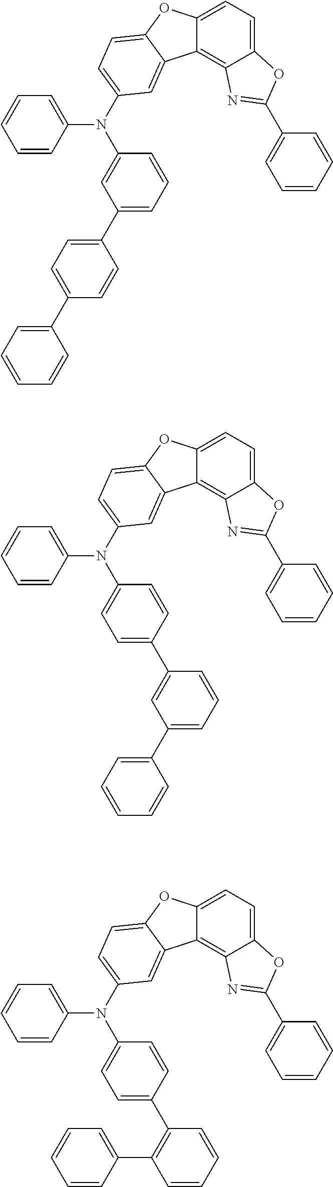 [SMILES: c1ccc(-c2ccc(-c3cccc(N(c4ccccc4)c4ccc5oc6ccc7oc(-c8ccccc8)nc7c6c5c4)c3)cc2)cc1 ; c1ccc(-c2cccc(-c3ccc(N(c4ccccc4)c4ccc5oc6ccc7oc(-c8ccccc8)nc7c6c5c4)cc3)c2)cc1 ; c1ccc(-c2nc3c(ccc4oc5ccc(N(c6ccccc6)c6ccc(-c7ccccc7-c7ccccc7)cc6)cc5c43)o2)cc1]

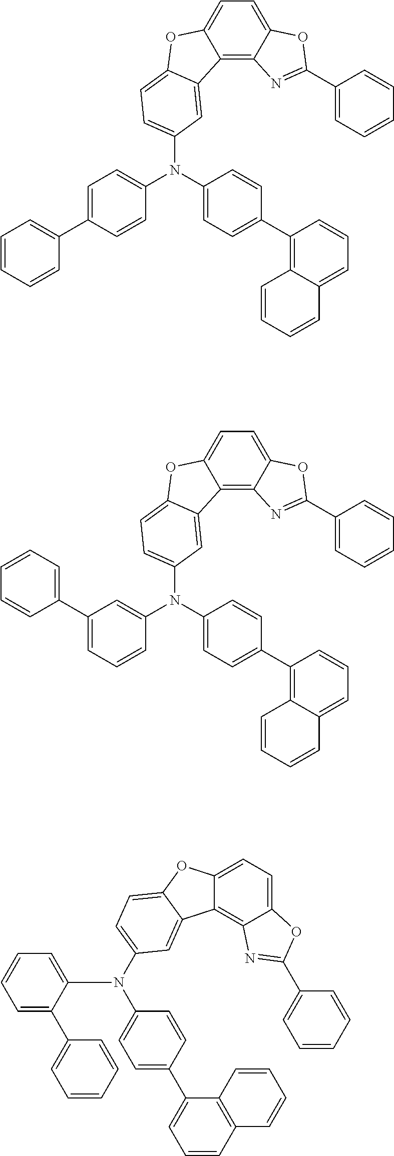 [SMILES: c1ccc(-c2ccc(N(c3ccc(-c4cccc5ccccc45)cc3)c3ccc4oc5ccc6oc(-c7ccccc7)nc6c5c4c3)cc2)cc1 ; c1ccc(-c2cccc(N(c3ccc(-c4cccc5ccccc45)cc3)c3ccc4oc5ccc6oc(-c7ccccc7)nc6c5c4c3)c2)cc1 ; c1ccc(-c2nc3c(ccc4oc5ccc(N(c6ccc(-c7cccc8ccccc78)cc6)c6ccccc6-c6ccccc6)cc5c43)o2)cc1]

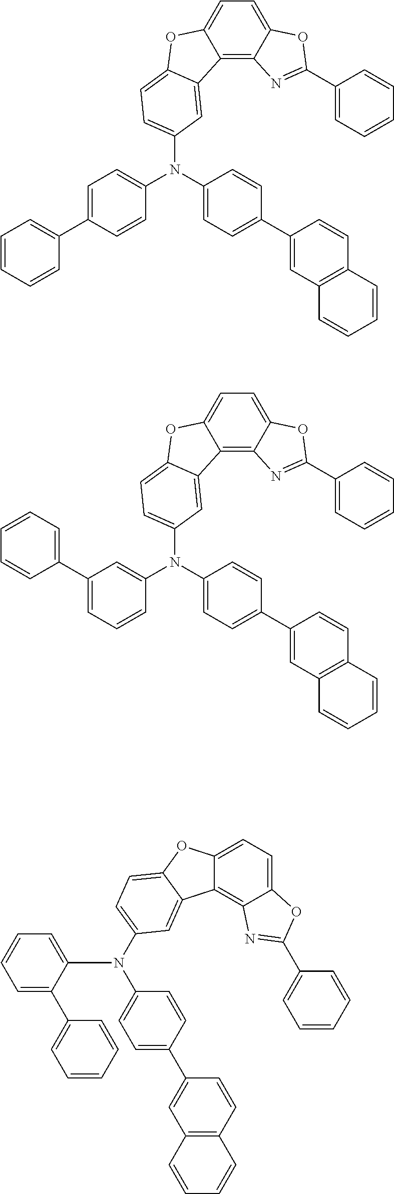 [SMILES: c1ccc(-c2ccc(N(c3ccc(-c4ccc5ccccc5c4)cc3)c3ccc4oc5ccc6oc(-c7ccccc7)nc6c5c4c3)cc2)cc1 ; c1ccc(-c2cccc(N(c3ccc(-c4ccc5ccccc5c4)cc3)c3ccc4oc5ccc6oc(-c7ccccc7)nc6c5c4c3)c2)cc1 ; c1ccc(-c2nc3c(ccc4oc5ccc(N(c6ccc(-c7ccc8ccccc8c7)cc6)c6ccccc6-c6ccccc6)cc5c43)o2)cc1]

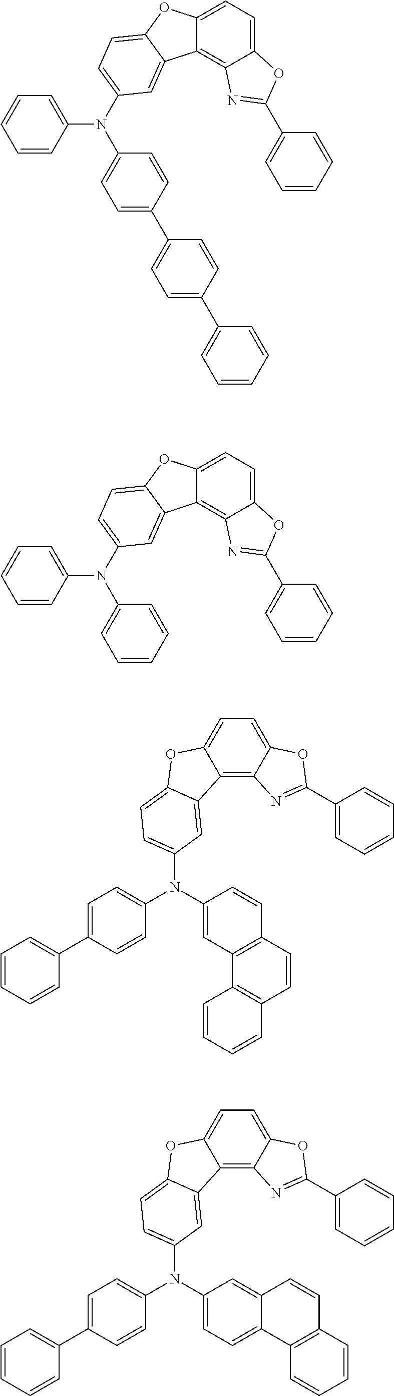 [SMILES: c1ccc(-c2ccc(-c3ccc(N(c4ccccc4)c4ccc5oc6ccc7oc(-c8ccccc8)nc7c6c5c4)cc3)cc2)cc1 ; c1ccc(-c2nc3c(ccc4oc5ccc(N(c6ccccc6)c6ccccc6)cc5c43)o2)cc1 ; c1ccc(-c2ccc(N(c3ccc4ccc5ccccc5c4c3)c3ccc4oc5ccc6oc(-c7ccccc7)nc6c5c4c3)cc2)cc1 ; c1ccc(-c2ccc(N(c3ccc4c(ccc5ccccc54)c3)c3ccc4oc5ccc6oc(-c7ccccc7)nc6c5c4c3)cc2)cc1]

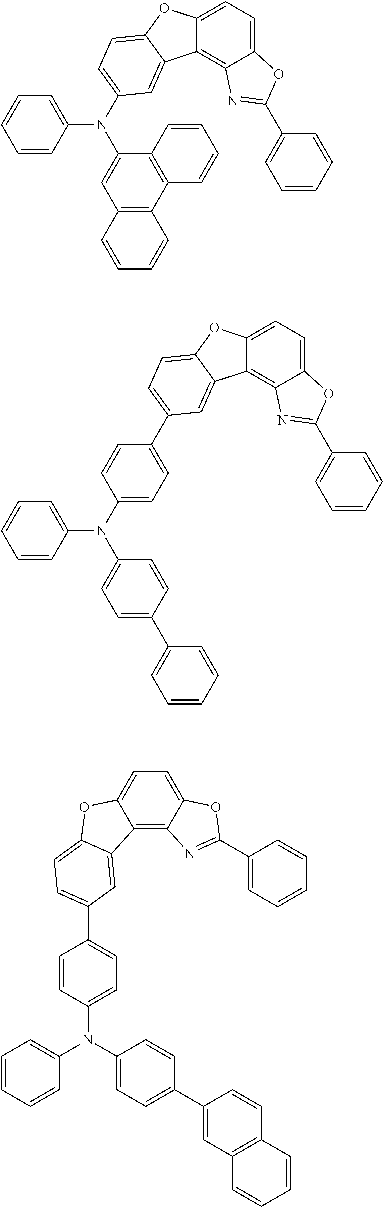 [SMILES: c1ccc(-c2nc3c(ccc4oc5ccc(N(c6ccccc6)c6cc7ccccc7c7ccccc67)cc5c43)o2)cc1 ; c1ccc(-c2ccc(N(c3ccccc3)c3ccc(-c4ccc5oc6ccc7oc(-c8ccccc8)nc7c6c5c4)cc3)cc2)cc1 ; c1ccc(-c2nc3c(ccc4oc5ccc(-c6ccc(N(c7ccccc7)c7ccc(-c8ccc9ccccc9c8)cc7)cc6)cc5c43)o2)cc1]

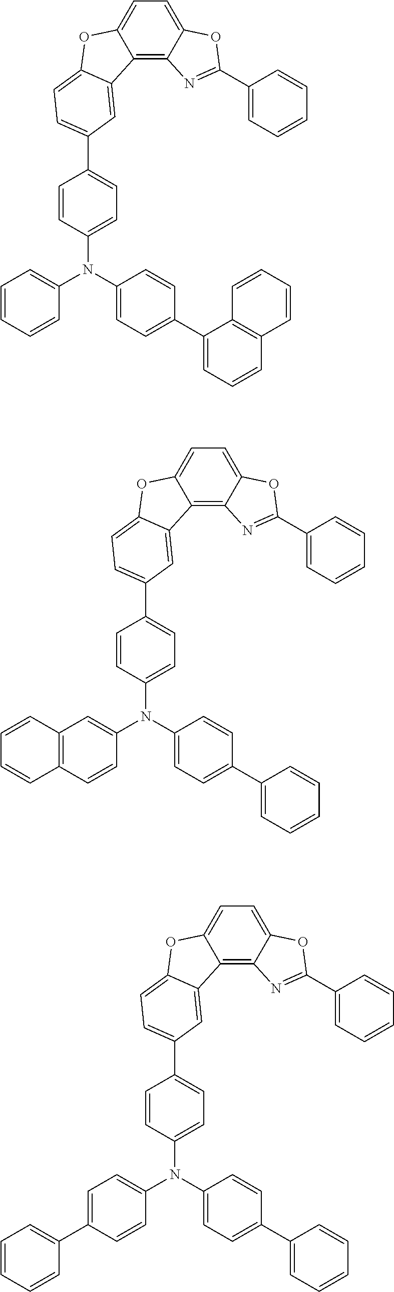 [SMILES: c1ccc(-c2nc3c(ccc4oc5ccc(-c6ccc(N(c7ccccc7)c7ccc(-c8cccc9ccccc89)cc7)cc6)cc5c43)o2)cc1 ; c1ccc(-c2ccc(N(c3ccc(-c4ccc5oc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5oc6ccc7oc(-c8ccccc8)nc7c6c5c4)cc3)cc2)cc1]

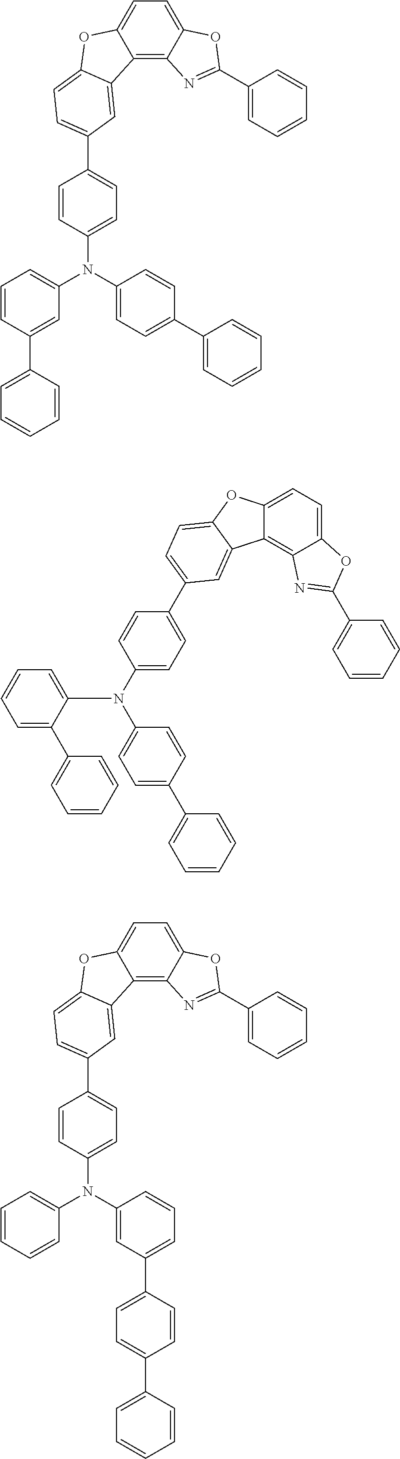 [SMILES: c1ccc(-c2ccc(N(c3ccc(-c4ccc5oc6ccc7oc(-c8ccccc8)nc7c6c5c4)cc3)c3cccc(-c4ccccc4)c3)cc2)cc1 ; c1ccc(-c2ccc(N(c3ccc(-c4ccc5oc6ccc7oc(-c8ccccc8)nc7c6c5c4)cc3)c3ccccc3-c3ccccc3)cc2)cc1 ; c1ccc(-c2ccc(-c3cccc(N(c4ccccc4)c4ccc(-c5ccc6oc7ccc8oc(-c9ccccc9)nc8c7c6c5)cc4)c3)cc2)cc1]

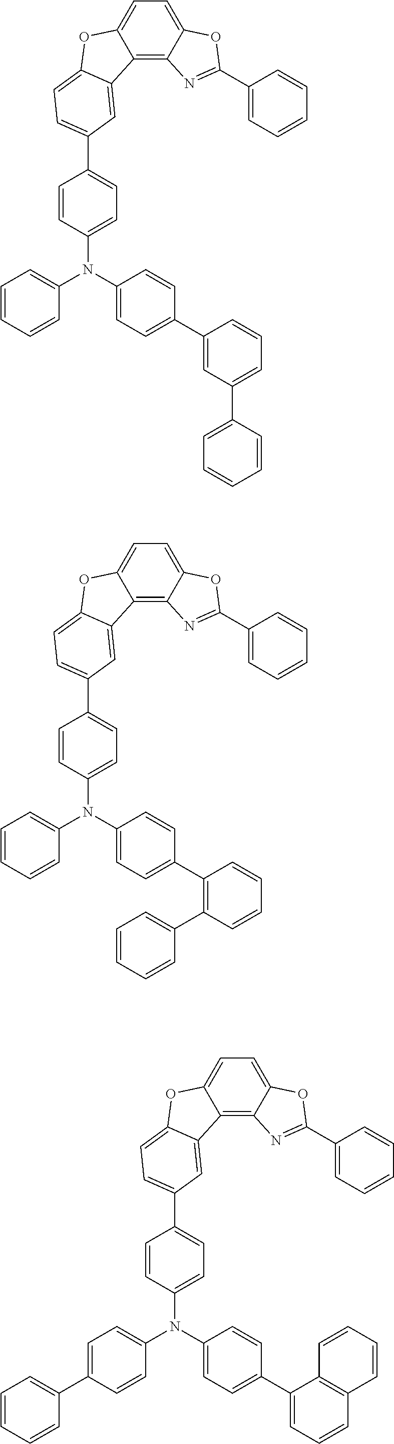 [SMILES: c1ccc(-c2cccc(-c3ccc(N(c4ccccc4)c4ccc(-c5ccc6oc7ccc8oc(-c9ccccc9)nc8c7c6c5)cc4)cc3)c2)cc1 ; c1ccc(-c2nc3c(ccc4oc5ccc(-c6ccc(N(c7ccccc7)c7ccc(-c8ccccc8-c8ccccc8)cc7)cc6)cc5c43)o2)cc1 ; c1ccc(-c2ccc(N(c3ccc(-c4ccc5oc6ccc7oc(-c8ccccc8)nc7c6c5c4)cc3)c3ccc(-c4cccc5ccccc45)cc3)cc2)cc1]

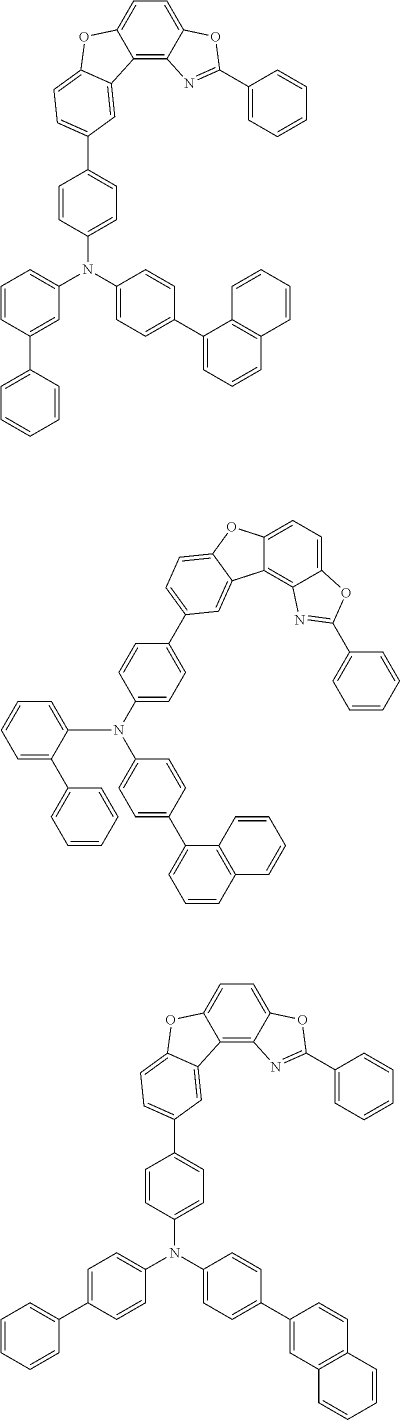 [SMILES: c1ccc(-c2cccc(N(c3ccc(-c4ccc5oc6ccc7oc(-c8ccccc8)nc7c6c5c4)cc3)c3ccc(-c4cccc5ccccc45)cc3)c2)cc1 ; c1ccc(-c2nc3c(ccc4oc5ccc(-c6ccc(N(c7ccc(-c8cccc9ccccc89)cc7)c7ccccc7-c7ccccc7)cc6)cc5c43)o2)cc1 ; c1ccc(-c2ccc(N(c3ccc(-c4ccc5ccccc5c4)cc3)c3ccc(-c4ccc5oc6ccc7oc(-c8ccccc8)nc7c6c5c4)cc3)cc2)cc1]

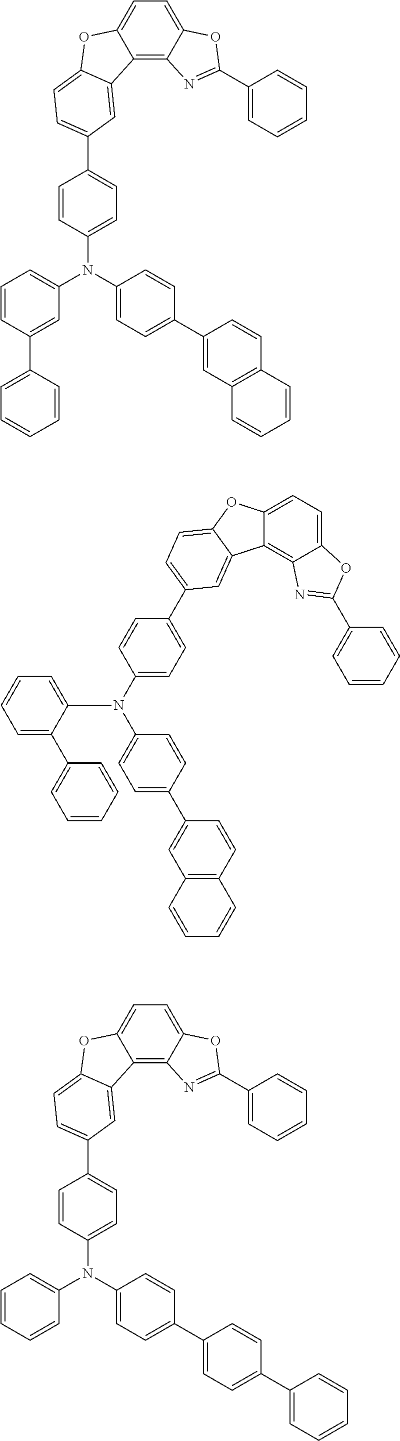 [SMILES: c1ccc(-c2cccc(N(c3ccc(-c4ccc5ccccc5c4)cc3)c3ccc(-c4ccc5oc6ccc7oc(-c8ccccc8)nc7c6c5c4)cc3)c2)cc1 ; c1ccc(-c2nc3c(ccc4oc5ccc(-c6ccc(N(c7ccc(-c8ccc9ccccc9c8)cc7)c7ccccc7-c7ccccc7)cc6)cc5c43)o2)cc1 ; c1ccc(-c2ccc(-c3ccc(N(c4ccccc4)c4ccc(-c5ccc6oc7ccc8oc(-c9ccccc9)nc8c7c6c5)cc4)cc3)cc2)cc1]

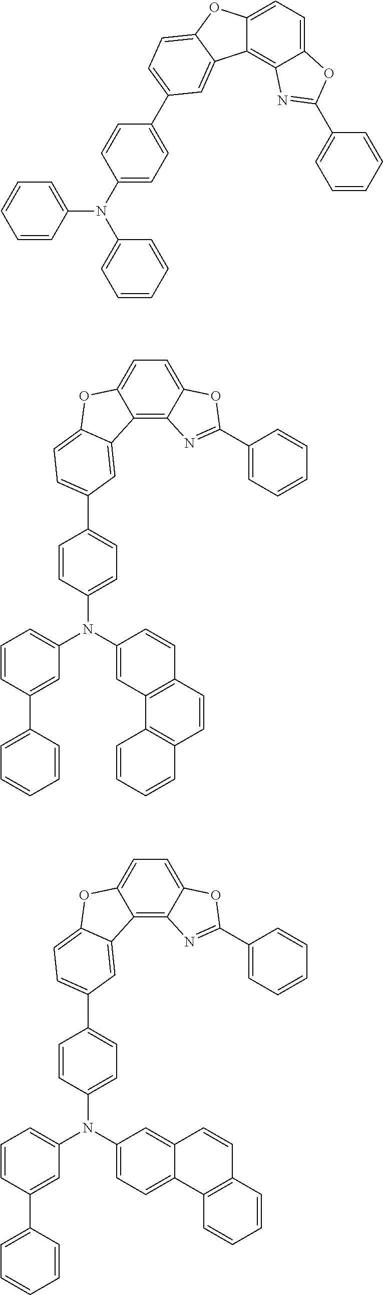 [SMILES: c1ccc(-c2nc3c(ccc4oc5ccc(-c6ccc(N(c7ccccc7)c7ccccc7)cc6)cc5c43)o2)cc1 ; c1ccc(-c2cccc(N(c3ccc(-c4ccc5oc6ccc7oc(-c8ccccc8)nc7c6c5c4)cc3)c3ccc4ccc5ccccc5c4c3)c2)cc1 ; c1ccc(-c2cccc(N(c3ccc(-c4ccc5oc6ccc7oc(-c8ccccc8)nc7c6c5c4)cc3)c3ccc4c(ccc5ccccc54)c3)c2)cc1]

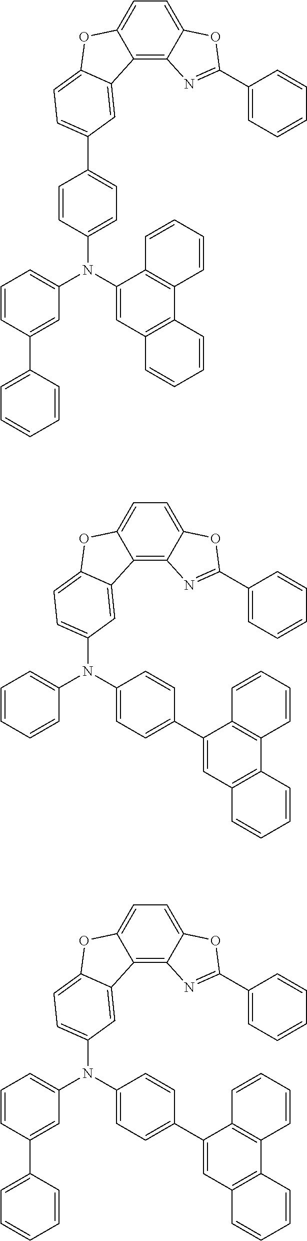 [SMILES: c1ccc(-c2cccc(N(c3ccc(-c4ccc5oc6ccc7oc(-c8ccccc8)nc7c6c5c4)cc3)c3cc4ccccc4c4ccccc34)c2)cc1 ; c1ccc(-c2nc3c(ccc4oc5ccc(N(c6ccccc6)c6ccc(-c7cc8ccccc8c8ccccc78)cc6)cc5c43)o2)cc1 ; c1ccc(-c2cccc(N(c3ccc(-c4cc5ccccc5c5ccccc45)cc3)c3ccc4oc5ccc6oc(-c7ccccc7)nc6c5c4c3)c2)cc1]

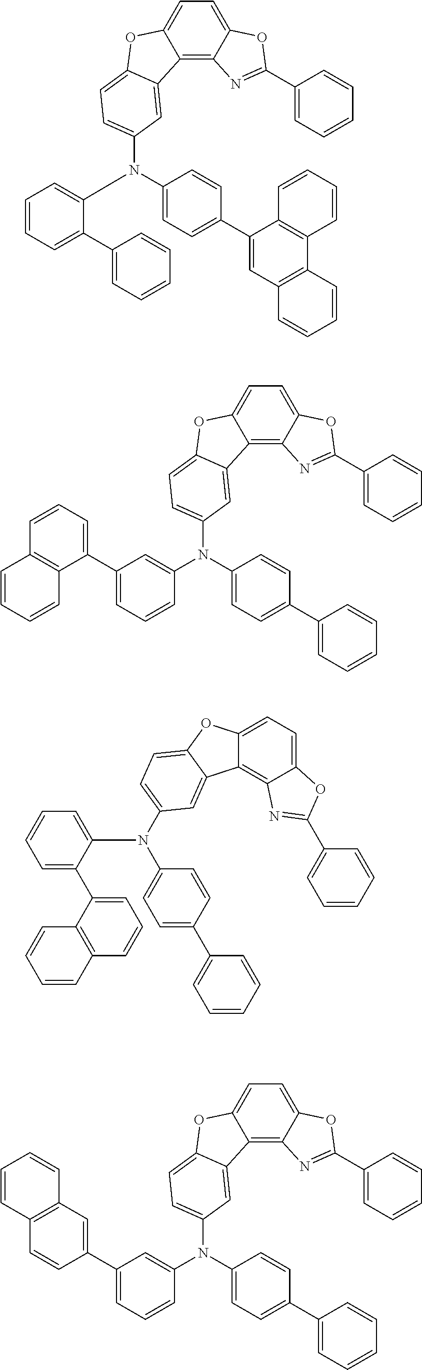 [SMILES: c1ccc(-c2nc3c(ccc4oc5ccc(N(c6ccc(-c7cc8ccccc8c8ccccc78)cc6)c6ccccc6-c6ccccc6)cc5c43)o2)cc1 ; c1ccc(-c2ccc(N(c3cccc(-c4cccc5ccccc45)c3)c3ccc4oc5ccc6oc(-c7ccccc7)nc6c5c4c3)cc2)cc1 ; c1ccc(-c2ccc(N(c3ccc4oc5ccc6oc(-c7ccccc7)nc6c5c4c3)c3ccccc3-c3cccc4ccccc34)cc2)cc1 ; c1ccc(-c2ccc(N(c3cccc(-c4ccc5ccccc5c4)c3)c3ccc4oc5ccc6oc(-c7ccccc7)nc6c5c4c3)cc2)cc1]

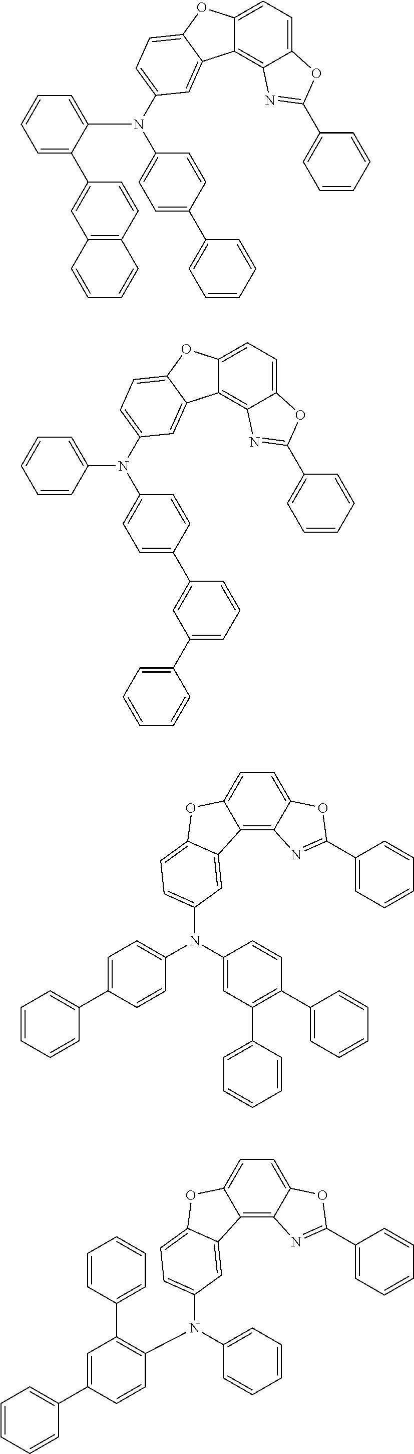

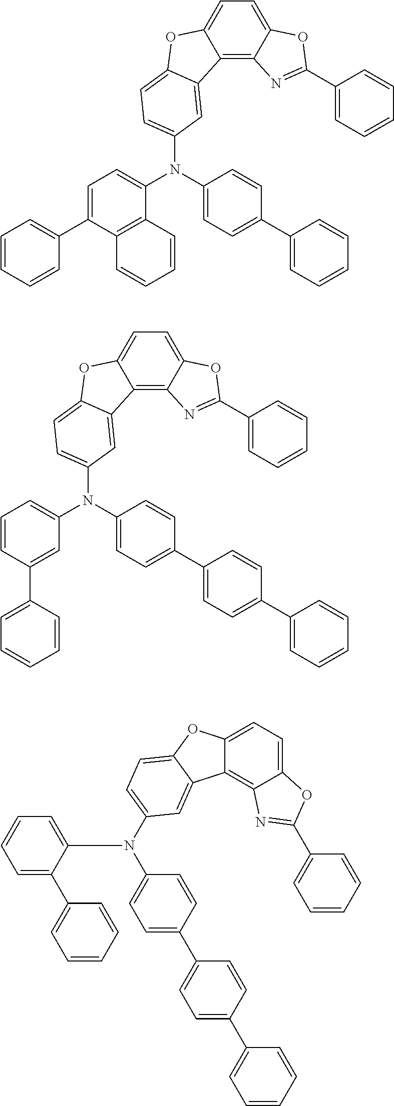 [SMILES: c1ccc(-c2ccc(N(c3ccc4oc5ccc6oc(-c7ccccc7)nc6c5c4c3)c3ccc(-c4ccccc4)c4ccccc34)cc2)cc1 ; c1ccc(-c2ccc(-c3ccc(N(c4cccc(-c5ccccc5)c4)c4ccc5oc6ccc7oc(-c8ccccc8)nc7c6c5c4)cc3)cc2)cc1 ; c1ccc(-c2ccc(-c3ccc(N(c4ccc5oc6ccc7oc(-c8ccccc8)nc7c6c5c4)c4ccccc4-c4ccccc4)cc3)cc2)cc1]

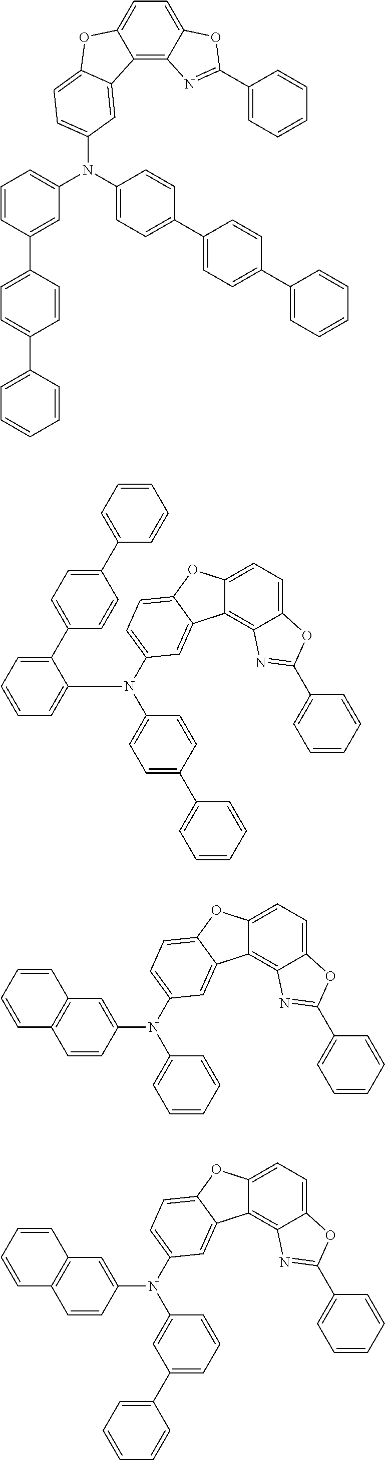 [SMILES: c1ccc(-c2ccc(-c3ccc(N(c4cccc(-c5ccc(-c6ccccc6)cc5)c4)c4ccc5oc6ccc7oc(-c8ccccc8)nc7c6c5c4)cc3)cc2)cc1 ; c1ccc(-c2ccc(-c3ccccc3N(c3ccc(-c4ccccc4)cc3)c3ccc4oc5ccc6oc(-c7ccccc7)nc6c5c4c3)cc2)cc1 ; c1ccc(-c2nc3c(ccc4oc5ccc(N(c6ccccc6)c6ccc7ccccc7c6)cc5c43)o2)cc1 ; c1ccc(-c2cccc(N(c3ccc4ccccc4c3)c3ccc4oc5ccc6oc(-c7ccccc7)nc6c5c4c3)c2)cc1]

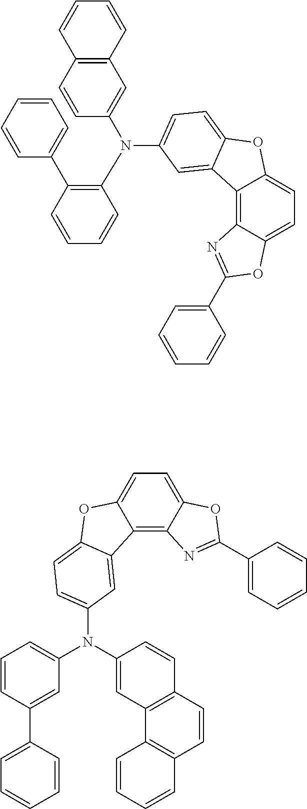 [SMILES: c1ccc(-c2nc3c(ccc4oc5ccc(N(c6ccc7ccccc7c6)c6ccccc6-c6ccccc6)cc5c43)o2)cc1 ; c1ccc(-c2cccc(N(c3ccc4ccc5ccccc5c4c3)c3ccc4oc5ccc6oc(-c7ccccc7)nc6c5c4c3)c2)cc1]

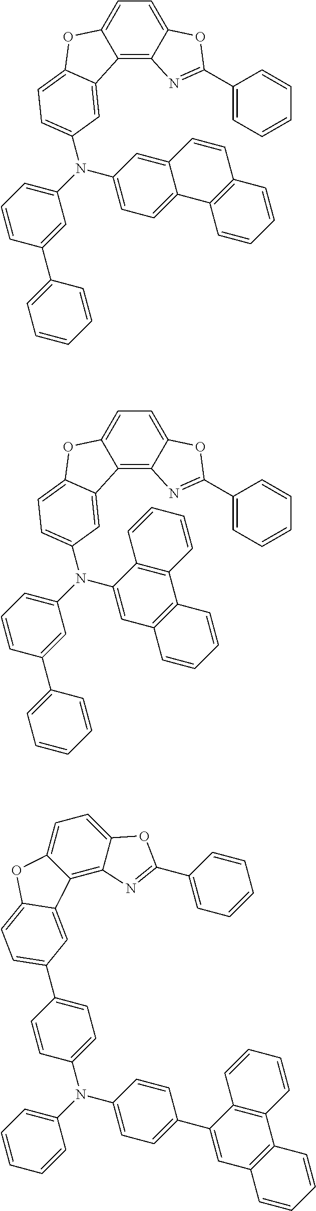 [SMILES: c1ccc(-c2cccc(N(c3ccc4c(ccc5ccccc54)c3)c3ccc4oc5ccc6oc(-c7ccccc7)nc6c5c4c3)c2)cc1 ; c1ccc(-c2cccc(N(c3ccc4oc5ccc6oc(-c7ccccc7)nc6c5c4c3)c3cc4ccccc4c4ccccc34)c2)cc1 ; c1ccc(-c2nc3c(ccc4oc5ccc(-c6ccc(N(c7ccccc7)c7ccc(-c8cc9ccccc9c9ccccc89)cc7)cc6)cc5c43)o2)cc1]

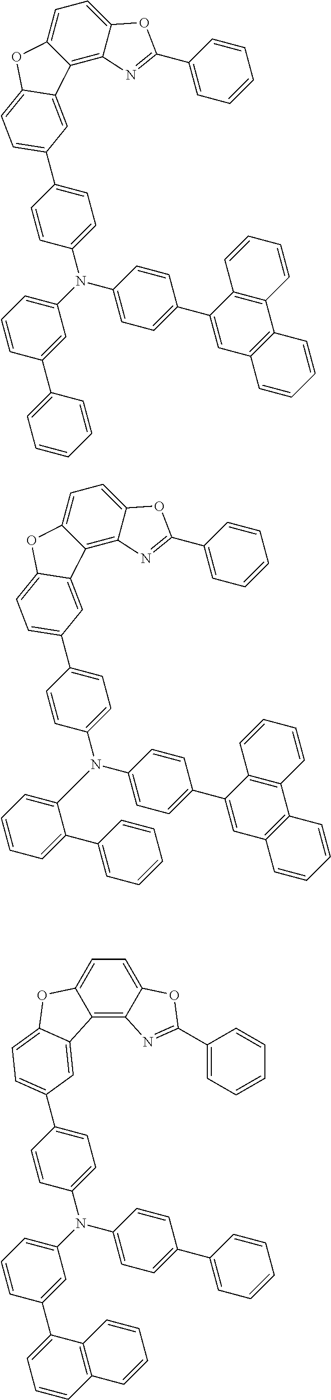

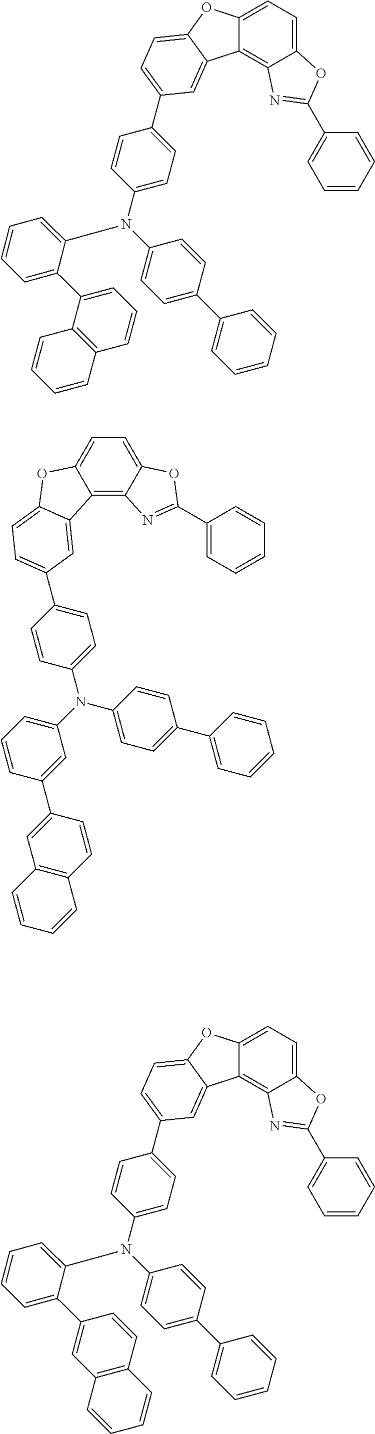 [SMILES: c1ccc(-c2ccc(N(c3ccc(-c4ccc5oc6ccc7oc(-c8ccccc8)nc7c6c5c4)cc3)c3ccccc3-c3cccc4ccccc34)cc2)cc1 ; c1ccc(-c2ccc(N(c3ccc(-c4ccc5oc6ccc7oc(-c8ccccc8)nc7c6c5c4)cc3)c3cccc(-c4ccc5ccccc5c4)c3)cc2)cc1 ; c1ccc(-c2ccc(N(c3ccc(-c4ccc5oc6ccc7oc(-c8ccccc8)nc7c6c5c4)cc3)c3ccccc3-c3ccc4ccccc4c3)cc2)cc1]

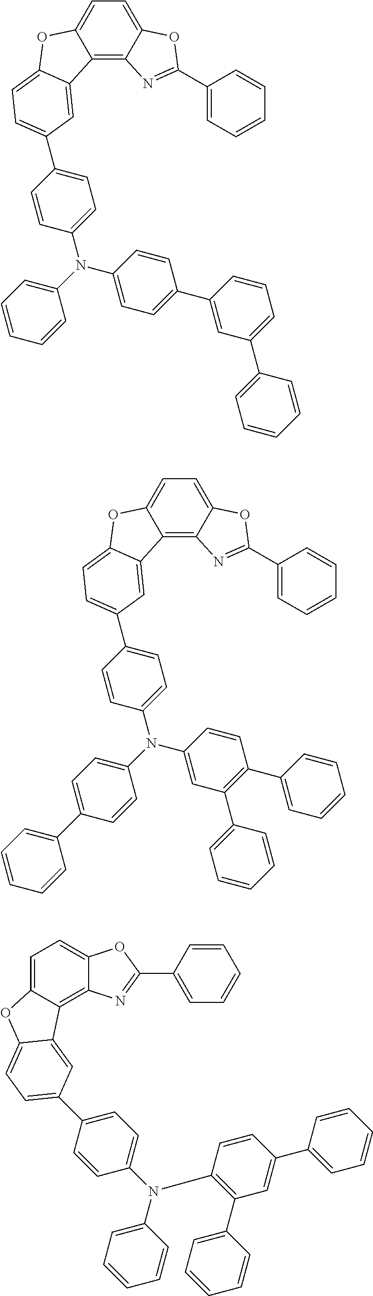 [SMILES: c1ccc(-c2cccc(-c3ccc(N(c4ccccc4)c4ccc(-c5ccc6oc7ccc8oc(-c9ccccc9)nc8c7c6c5)cc4)cc3)c2)cc1 ; c1ccc(-c2ccc(N(c3ccc(-c4ccc5oc6ccc7oc(-c8ccccc8)nc7c6c5c4)cc3)c3ccc(-c4ccccc4)c(-c4ccccc4)c3)cc2)cc1 ; c1ccc(-c2ccc(N(c3ccccc3)c3ccc(-c4ccc5oc6ccc7oc(-c8ccccc8)nc7c6c5c4)cc3)c(-c3ccccc3)c2)cc1]

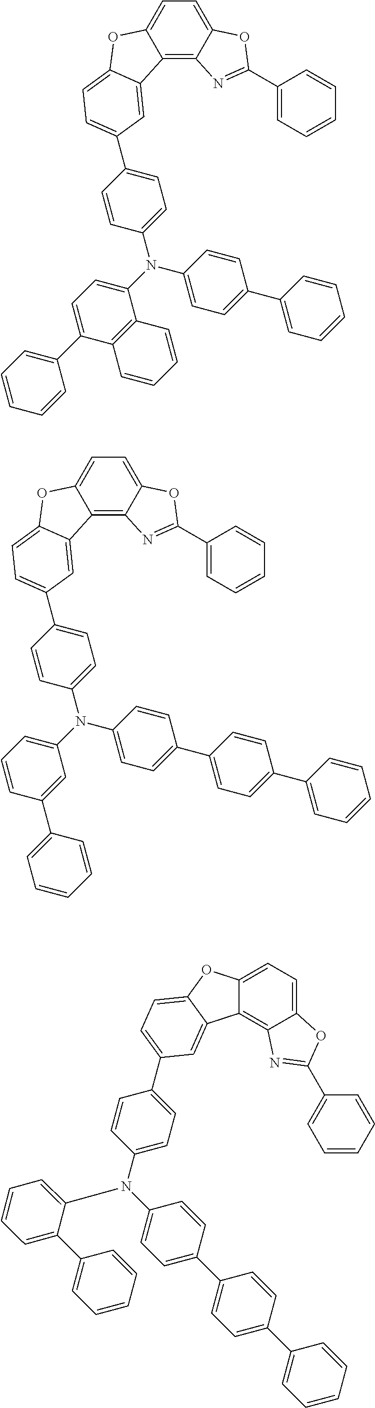 [SMILES: c1ccc(-c2ccc(N(c3ccc(-c4ccc5oc6ccc7oc(-c8ccccc8)nc7c6c5c4)cc3)c3ccc(-c4ccccc4)c4ccccc34)cc2)cc1 ; c1ccc(-c2ccc(-c3ccc(N(c4ccc(-c5ccc6oc7ccc8oc(-c9ccccc9)nc8c7c6c5)cc4)c4cccc(-c5ccccc5)c4)cc3)cc2)cc1 ; c1ccc(-c2ccc(-c3ccc(N(c4ccc(-c5ccc6oc7ccc8oc(-c9ccccc9)nc8c7c6c5)cc4)c4ccccc4-c4ccccc4)cc3)cc2)cc1]

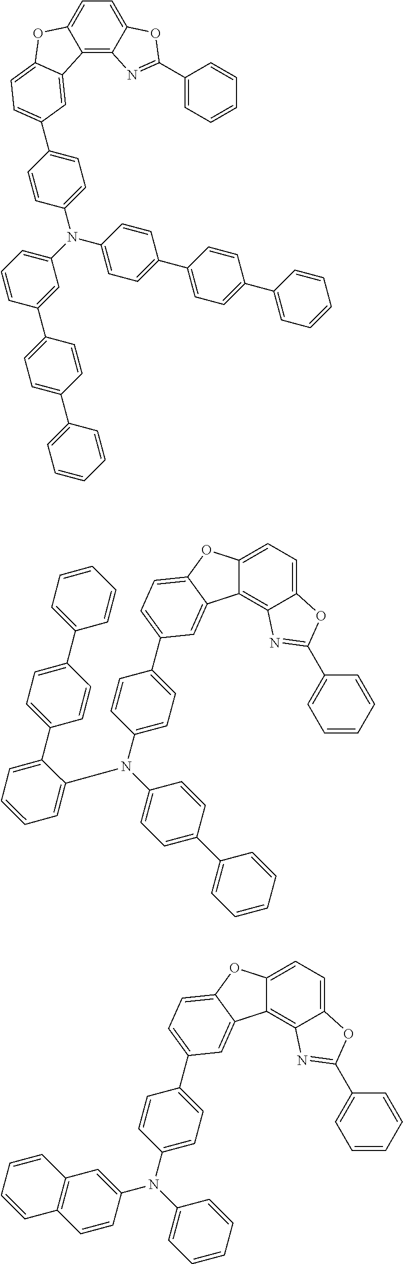 [SMILES: c1ccc(-c2ccc(-c3ccc(N(c4ccc(-c5ccc6oc7ccc8oc(-c9ccccc9)nc8c7c6c5)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4ccc5oc6ccc7oc(-c8ccccc8)nc7c6c5c4)cc3)cc2)cc1 ; c1ccc(-c2nc3c(ccc4oc5ccc(-c6ccc(N(c7ccccc7)c7ccc8ccccc8c7)cc6)cc5c43)o2)cc1]

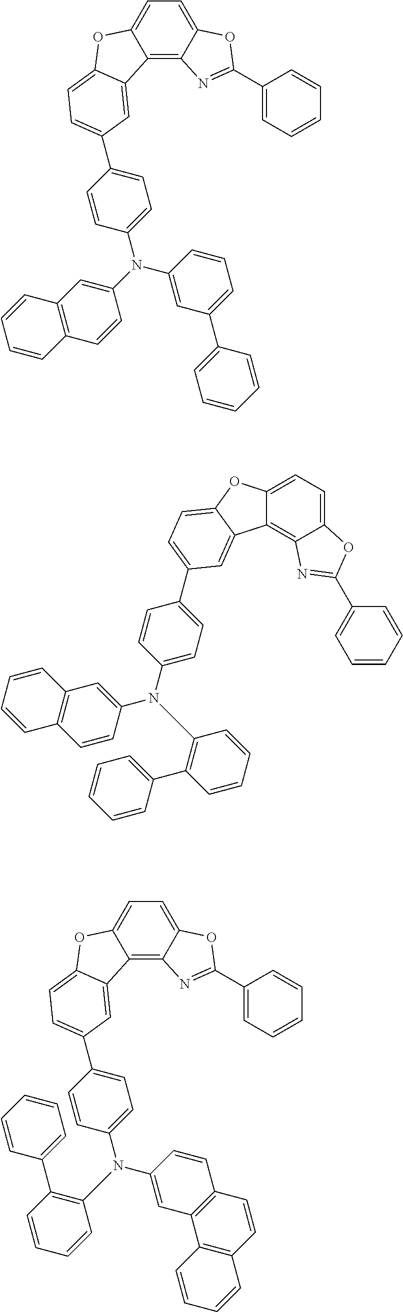 [SMILES: c1ccc(-c2cccc(N(c3ccc(-c4ccc5oc6ccc7oc(-c8ccccc8)nc7c6c5c4)cc3)c3ccc4ccccc4c3)c2)cc1 ; c1ccc(-c2nc3c(ccc4oc5ccc(-c6ccc(N(c7ccc8ccccc8c7)c7ccccc7-c7ccccc7)cc6)cc5c43)o2)cc1 ; c1ccc(-c2nc3c(ccc4oc5ccc(-c6ccc(N(c7ccc8ccc9ccccc9c8c7)c7ccccc7-c7ccccc7)cc6)cc5c43)o2)cc1]

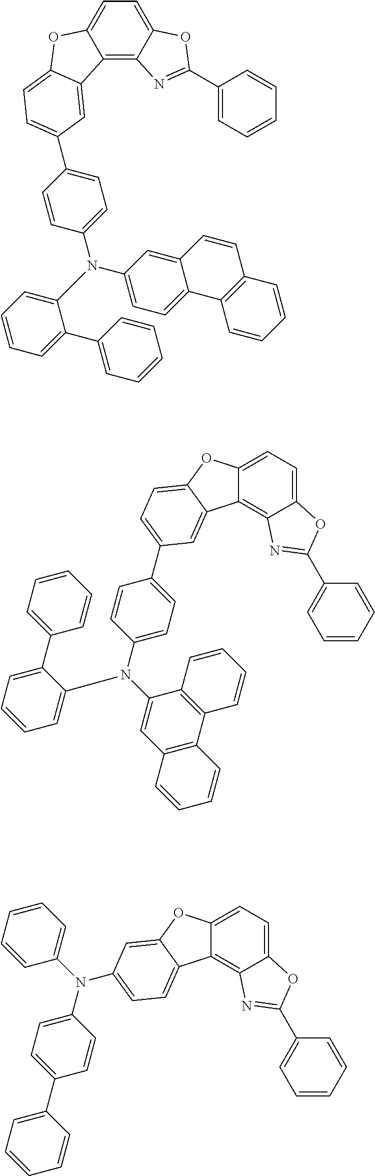 [SMILES: c1ccc(-c2nc3c(ccc4oc5ccc(-c6ccc(N(c7ccc8c(ccc9ccccc98)c7)c7ccccc7-c7ccccc7)cc6)cc5c43)o2)cc1 ; c1ccc(-c2nc3c(ccc4oc5ccc(-c6ccc(N(c7ccccc7-c7ccccc7)c7cc8ccccc8c8ccccc78)cc6)cc5c43)o2)cc1 ; c1ccc(-c2ccc(N(c3ccccc3)c3ccc4c(c3)oc3ccc5oc(-c6ccccc6)nc5c34)cc2)cc1]

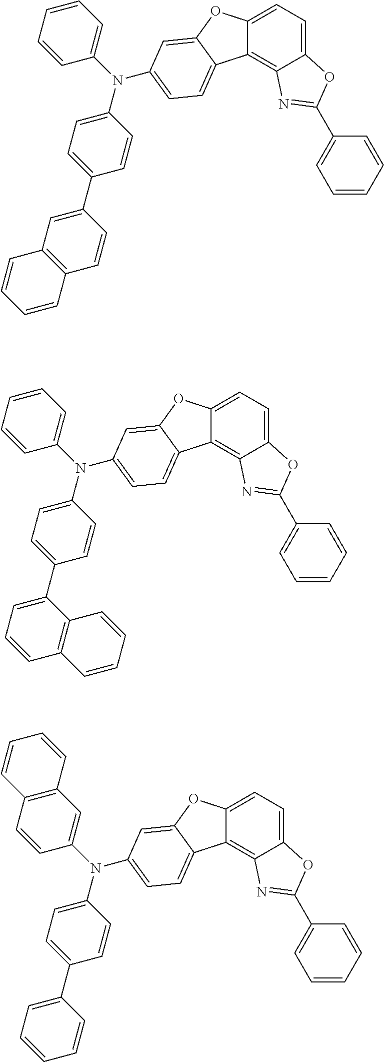 [SMILES: c1ccc(-c2nc3c(ccc4oc5cc(N(c6ccccc6)c6ccc(-c7ccc8ccccc8c7)cc6)ccc5c43)o2)cc1 ; c1ccc(-c2nc3c(ccc4oc5cc(N(c6ccccc6)c6ccc(-c7cccc8ccccc78)cc6)ccc5c43)o2)cc1 ; c1ccc(-c2ccc(N(c3ccc4ccccc4c3)c3ccc4c(c3)oc3ccc5oc(-c6ccccc6)nc5c34)cc2)cc1]

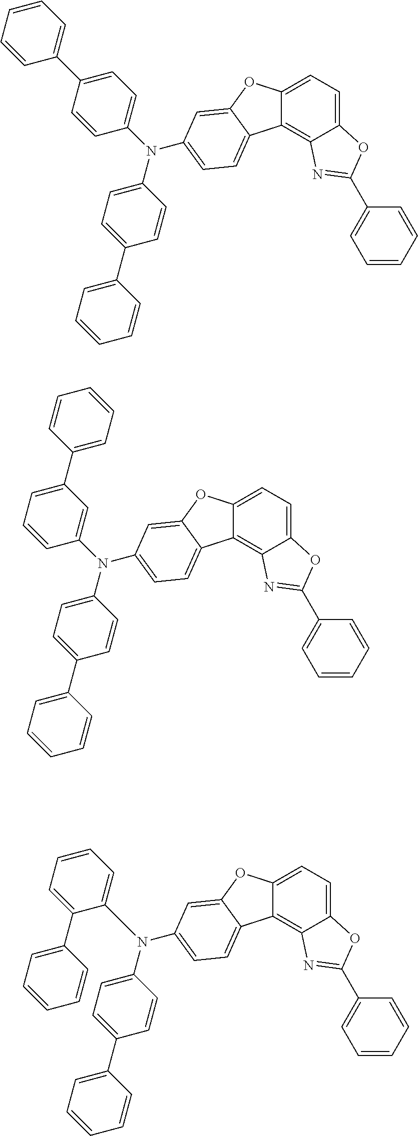

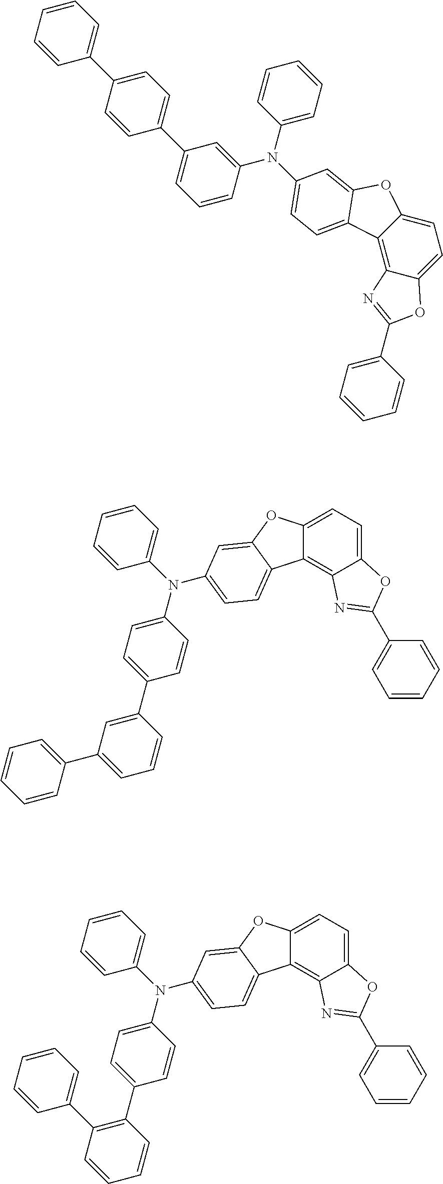 [SMILES: c1ccc(-c2ccc(-c3cccc(N(c4ccccc4)c4ccc5c(c4)oc4ccc6oc(-c7ccccc7)nc6c45)c3)cc2)cc1 ; c1ccc(-c2cccc(-c3ccc(N(c4ccccc4)c4ccc5c(c4)oc4ccc6oc(-c7ccccc7)nc6c45)cc3)c2)cc1 ; c1ccc(-c2nc3c(ccc4oc5cc(N(c6ccccc6)c6ccc(-c7ccccc7-c7ccccc7)cc6)ccc5c43)o2)cc1]

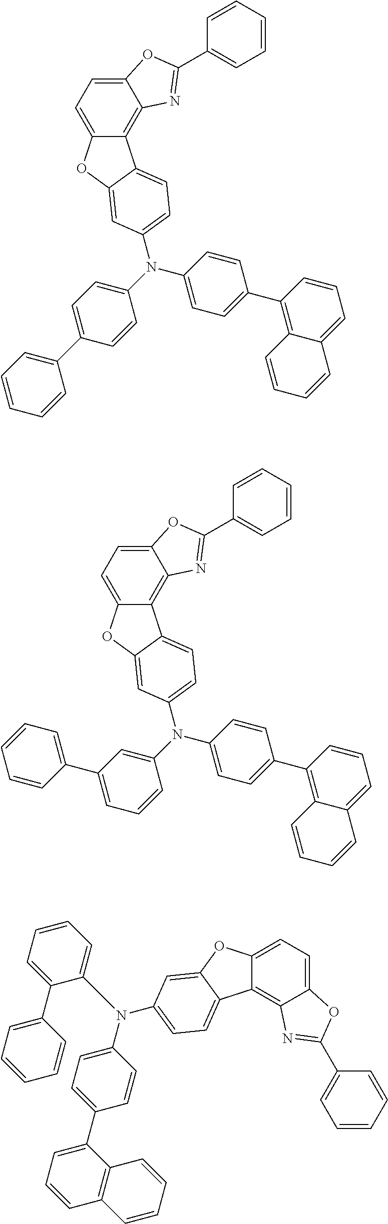 [SMILES: c1ccc(-c2ccc(N(c3ccc(-c4cccc5ccccc45)cc3)c3ccc4c(c3)oc3ccc5oc(-c6ccccc6)nc5c34)cc2)cc1 ; c1ccc(-c2cccc(N(c3ccc(-c4cccc5ccccc45)cc3)c3ccc4c(c3)oc3ccc5oc(-c6ccccc6)nc5c34)c2)cc1 ; c1ccc(-c2nc3c(ccc4oc5cc(N(c6ccc(-c7cccc8ccccc78)cc6)c6ccccc6-c6ccccc6)ccc5c43)o2)cc1]

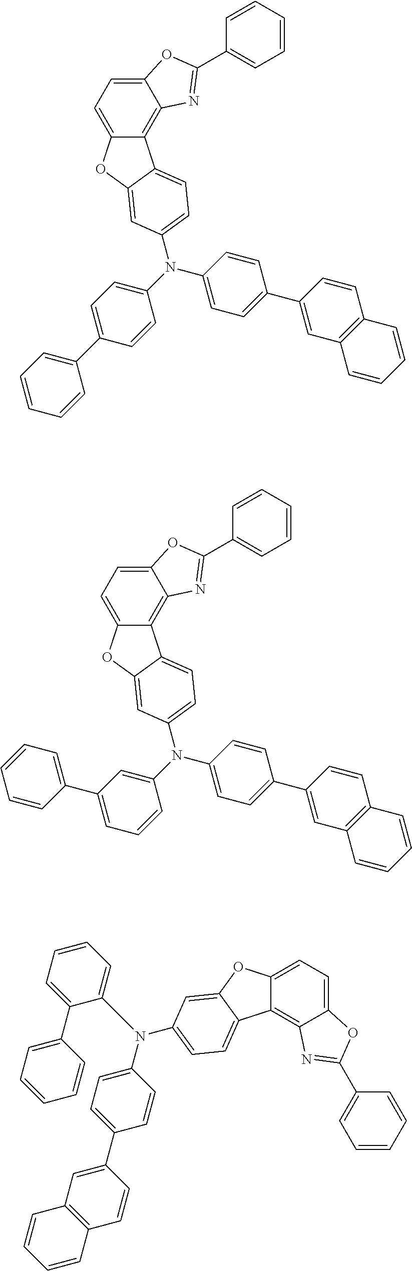 [SMILES: c1ccc(-c2ccc(N(c3ccc(-c4ccc5ccccc5c4)cc3)c3ccc4c(c3)oc3ccc5oc(-c6ccccc6)nc5c34)cc2)cc1 ; c1ccc(-c2cccc(N(c3ccc(-c4ccc5ccccc5c4)cc3)c3ccc4c(c3)oc3ccc5oc(-c6ccccc6)nc5c34)c2)cc1 ; c1ccc(-c2nc3c(ccc4oc5cc(N(c6ccc(-c7ccc8ccccc8c7)cc6)c6ccccc6-c6ccccc6)ccc5c43)o2)cc1]

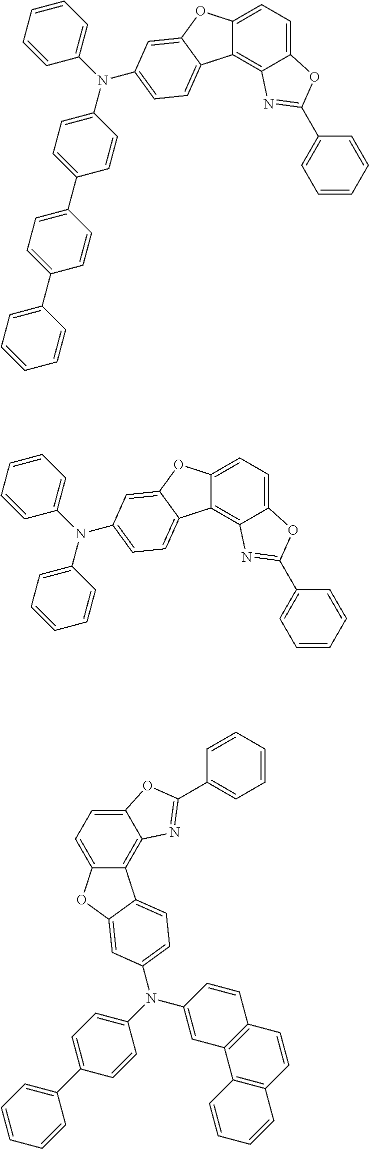 [SMILES: c1ccc(-c2ccc(-c3ccc(N(c4ccccc4)c4ccc5c(c4)oc4ccc6oc(-c7ccccc7)nc6c45)cc3)cc2)cc1 ; c1ccc(-c2nc3c(ccc4oc5cc(N(c6ccccc6)c6ccccc6)ccc5c43)o2)cc1 ; c1ccc(-c2ccc(N(c3ccc4c(c3)oc3ccc5oc(-c6ccccc6)nc5c34)c3ccc4ccc5ccccc5c4c3)cc2)cc1]

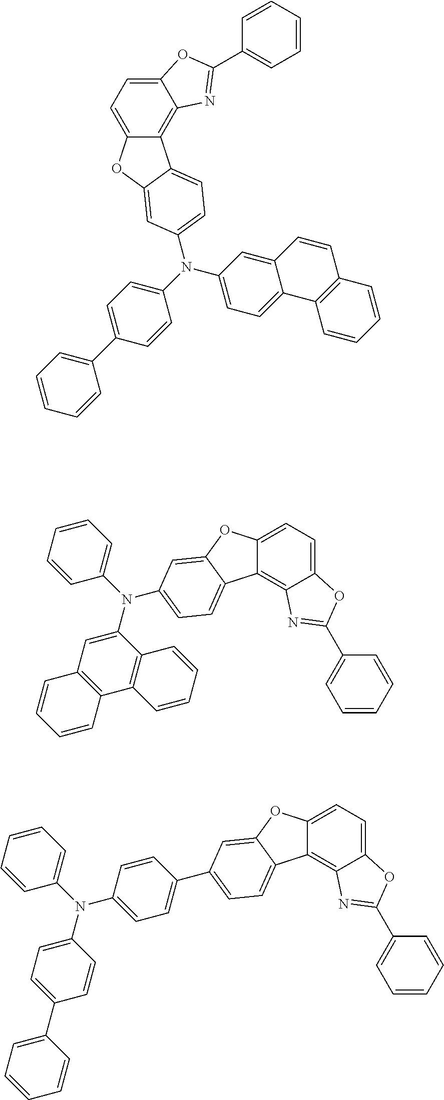 [SMILES: c1ccc(-c2ccc(N(c3ccc4c(ccc5ccccc54)c3)c3ccc4c(c3)oc3ccc5oc(-c6ccccc6)nc5c34)cc2)cc1 ; c1ccc(-c2nc3c(ccc4oc5cc(N(c6ccccc6)c6cc7ccccc7c7ccccc67)ccc5c43)o2)cc1 ; c1ccc(-c2ccc(N(c3ccccc3)c3ccc(-c4ccc5c(c4)oc4ccc6oc(-c7ccccc7)nc6c45)cc3)cc2)cc1]

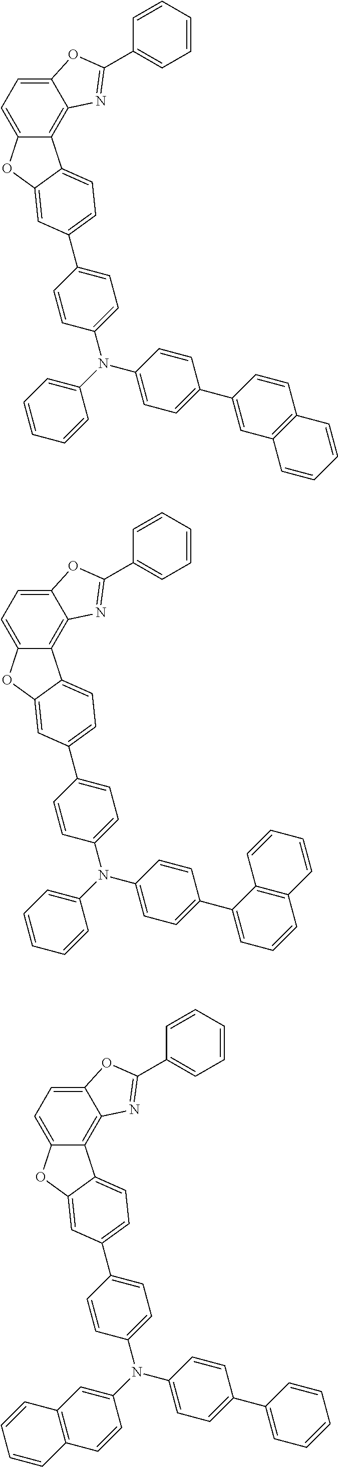 [SMILES: c1ccc(-c2nc3c(ccc4oc5cc(-c6ccc(N(c7ccccc7)c7ccc(-c8ccc9ccccc9c8)cc7)cc6)ccc5c43)o2)cc1 ; c1ccc(-c2nc3c(ccc4oc5cc(-c6ccc(N(c7ccccc7)c7ccc(-c8cccc9ccccc89)cc7)cc6)ccc5c43)o2)cc1 ; c1ccc(-c2ccc(N(c3ccc(-c4ccc5c(c4)oc4ccc6oc(-c7ccccc7)nc6c45)cc3)c3ccc4ccccc4c3)cc2)cc1]

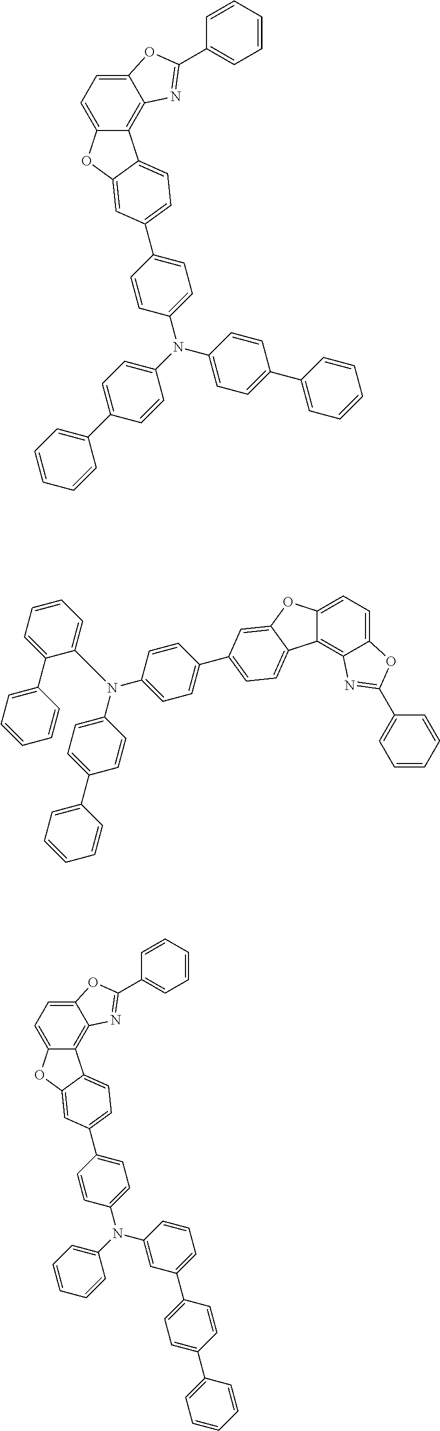 [SMILES: c1ccc(-c2ccc(N(c3ccc(-c4ccccc4)cc3)c3ccc(-c4ccc5c(c4)oc4ccc6oc(-c7ccccc7)nc6c45)cc3)cc2)cc1 ; c1ccc(-c2ccc(N(c3ccc(-c4ccc5c(c4)oc4ccc6oc(-c7ccccc7)nc6c45)cc3)c3ccccc3-c3ccccc3)cc2)cc1 ; c1ccc(-c2ccc(-c3cccc(N(c4ccccc4)c4ccc(-c5ccc6c(c5)oc5ccc7oc(-c8ccccc8)nc7c56)cc4)c3)cc2)cc1]

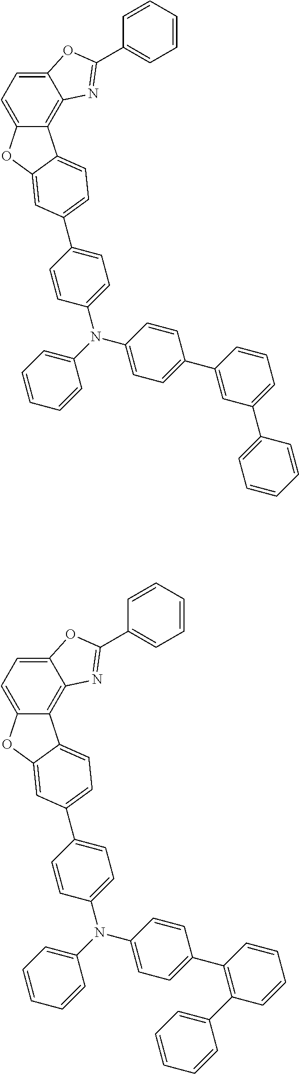 [SMILES: c1ccc(-c2cccc(-c3ccc(N(c4ccccc4)c4ccc(-c5ccc6c(c5)oc5ccc7oc(-c8ccccc8)nc7c56)cc4)cc3)c2)cc1 ; c1ccc(-c2nc3c(ccc4oc5cc(-c6ccc(N(c7ccccc7)c7ccc(-c8ccccc8-c8ccccc8)cc7)cc6)ccc5c43)o2)cc1]

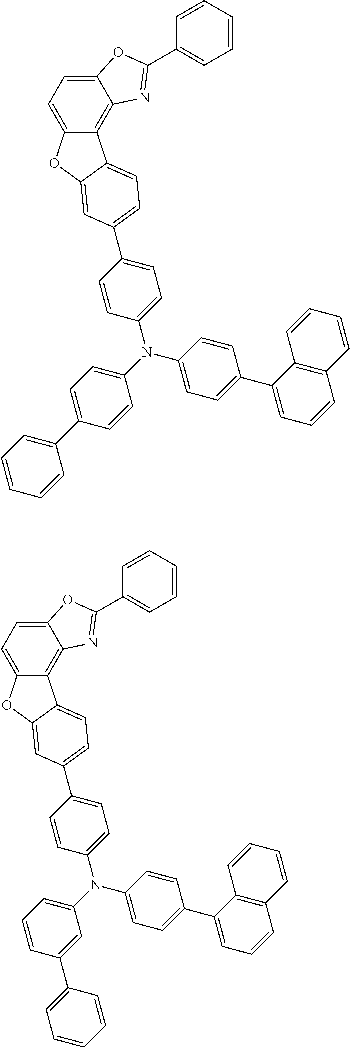 [SMILES: c1ccc(-c2ccc(N(c3ccc(-c4ccc5c(c4)oc4ccc6oc(-c7ccccc7)nc6c45)cc3)c3ccc(-c4cccc5ccccc45)cc3)cc2)cc1 ; c1ccc(-c2cccc(N(c3ccc(-c4ccc5c(c4)oc4ccc6oc(-c7ccccc7)nc6c45)cc3)c3ccc(-c4cccc5ccccc45)cc3)c2)cc1]

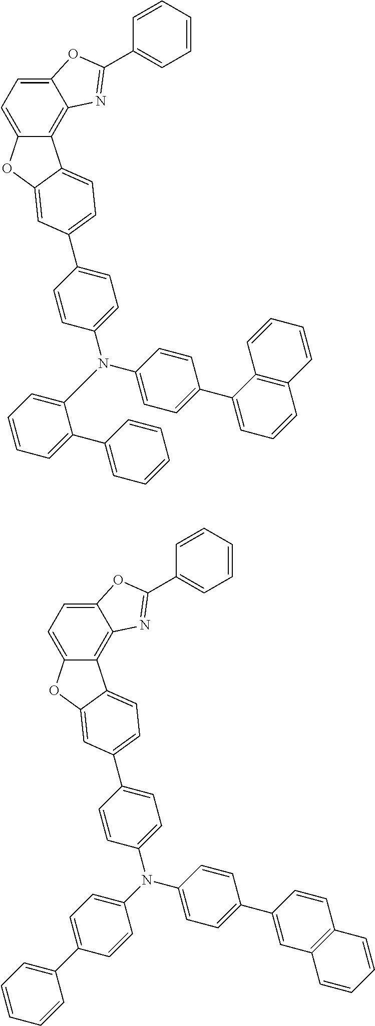 [SMILES: c1ccc(-c2nc3c(ccc4oc5cc(-c6ccc(N(c7ccc(-c8cccc9ccccc89)cc7)c7ccccc7-c7ccccc7)cc6)ccc5c43)o2)cc1 ; c1ccc(-c2ccc(N(c3ccc(-c4ccc5ccccc5c4)cc3)c3ccc(-c4ccc5c(c4)oc4ccc6oc(-c7ccccc7)nc6c45)cc3)cc2)cc1]

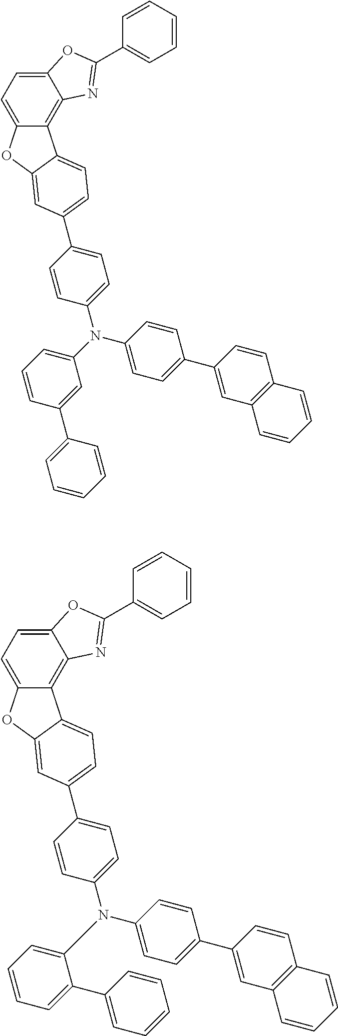 [SMILES: c1ccc(-c2cccc(N(c3ccc(-c4ccc5ccccc5c4)cc3)c3ccc(-c4ccc5c(c4)oc4ccc6oc(-c7ccccc7)nc6c45)cc3)c2)cc1 ; c1ccc(-c2nc3c(ccc4oc5cc(-c6ccc(N(c7ccc(-c8ccc9ccccc9c8)cc7)c7ccccc7-c7ccccc7)cc6)ccc5c43)o2)cc1]

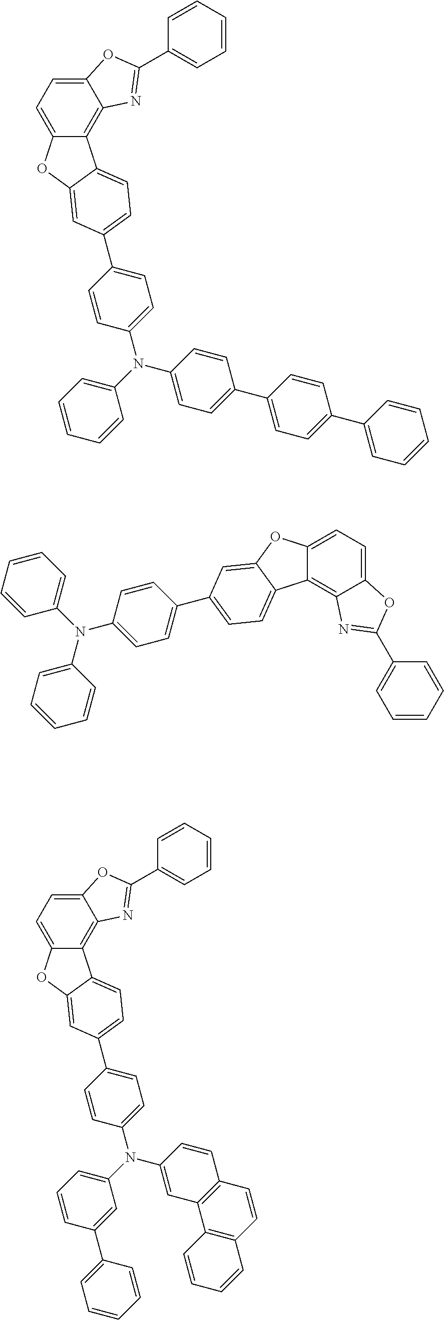 [SMILES: c1ccc(-c2ccc(-c3ccc(N(c4ccccc4)c4ccc(-c5ccc6c(c5)oc5ccc7oc(-c8ccccc8)nc7c56)cc4)cc3)cc2)cc1 ; c1ccc(-c2nc3c(ccc4oc5cc(-c6ccc(N(c7ccccc7)c7ccccc7)cc6)ccc5c43)o2)cc1 ; c1ccc(-c2cccc(N(c3ccc(-c4ccc5c(c4)oc4ccc6oc(-c7ccccc7)nc6c45)cc3)c3ccc4ccc5ccccc5c4c3)c2)cc1]

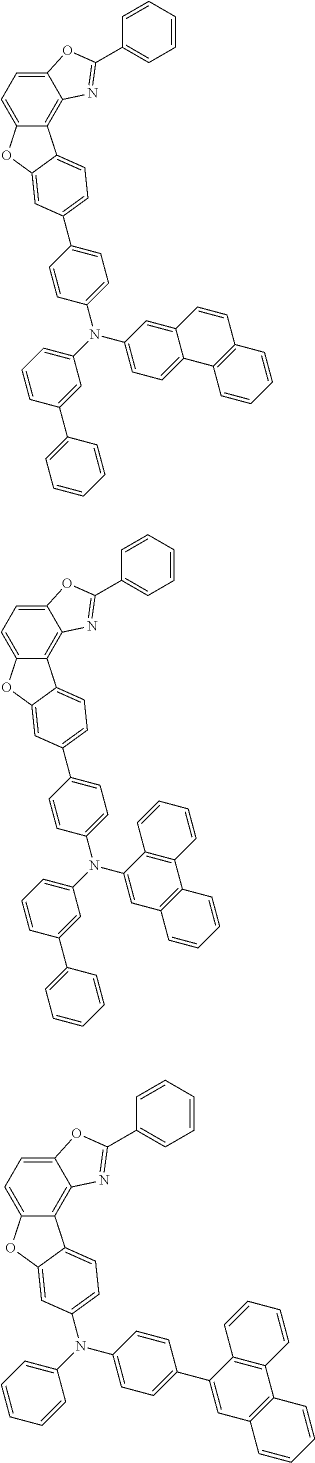 [SMILES: c1ccc(-c2cccc(N(c3ccc(-c4ccc5c(c4)oc4ccc6oc(-c7ccccc7)nc6c45)cc3)c3ccc4c(ccc5ccccc54)c3)c2)cc1 ; c1ccc(-c2cccc(N(c3ccc(-c4ccc5c(c4)oc4ccc6oc(-c7ccccc7)nc6c45)cc3)c3cc4ccccc4c4ccccc34)c2)cc1 ; c1ccc(-c2nc3c(ccc4oc5cc(N(c6ccccc6)c6ccc(-c7cc8ccccc8c8ccccc78)cc6)ccc5c43)o2)cc1]

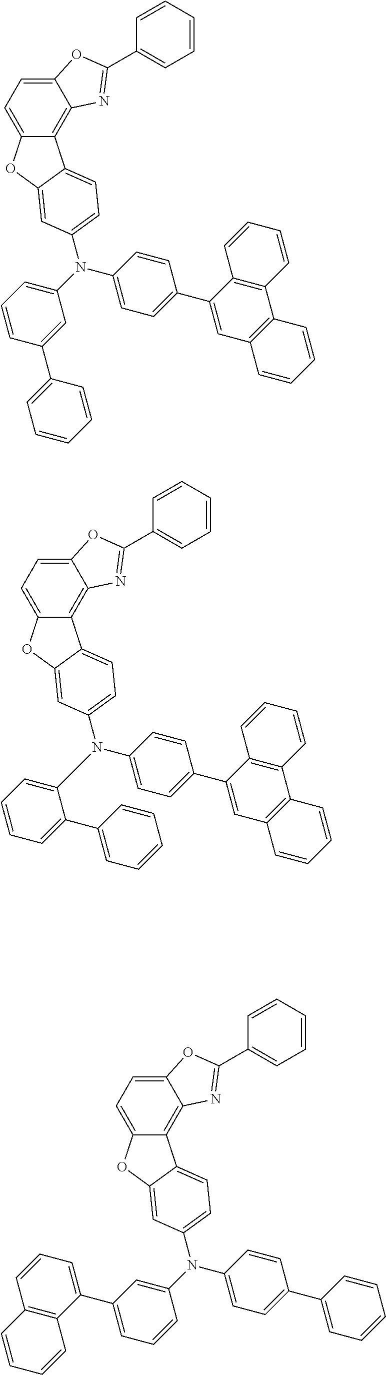 [SMILES: c1ccc(-c2cccc(N(c3ccc(-c4cc5ccccc5c5ccccc45)cc3)c3ccc4c(c3)oc3ccc5oc(-c6ccccc6)nc5c34)c2)cc1 ; c1ccc(-c2nc3c(ccc4oc5cc(N(c6ccc(-c7cc8ccccc8c8ccccc78)cc6)c6ccccc6-c6ccccc6)ccc5c43)o2)cc1 ; c1ccc(-c2ccc(N(c3cccc(-c4cccc5ccccc45)c3)c3ccc4c(c3)oc3ccc5oc(-c6ccccc6)nc5c34)cc2)cc1]

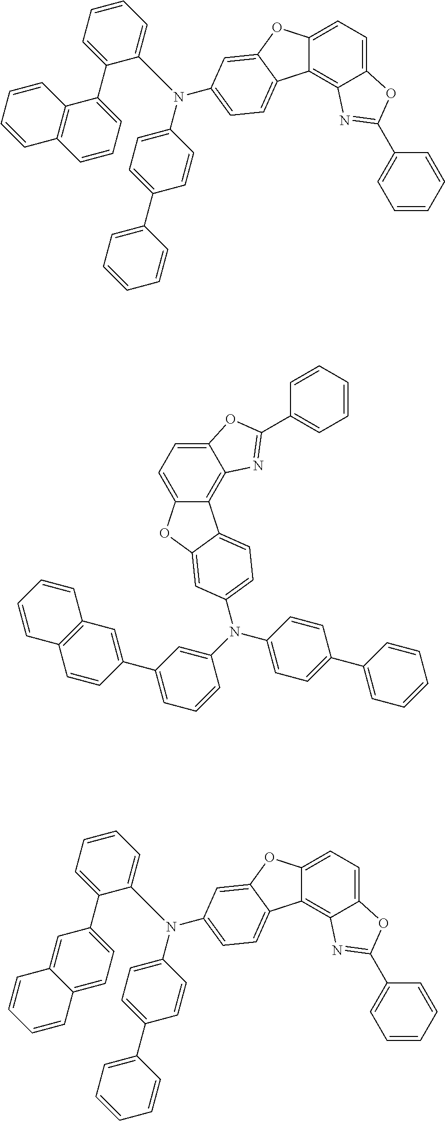 [SMILES: c1ccc(-c2ccc(N(c3ccc4c(c3)oc3ccc5oc(-c6ccccc6)nc5c34)c3ccccc3-c3cccc4ccccc34)cc2)cc1 ; c1ccc(-c2ccc(N(c3cccc(-c4ccc5ccccc5c4)c3)c3ccc4c(c3)oc3ccc5oc(-c6ccccc6)nc5c34)cc2)cc1 ; c1ccc(-c2ccc(N(c3ccc4c(c3)oc3ccc5oc(-c6ccccc6)nc5c34)c3ccccc3-c3ccc4ccccc4c3)cc2)cc1]

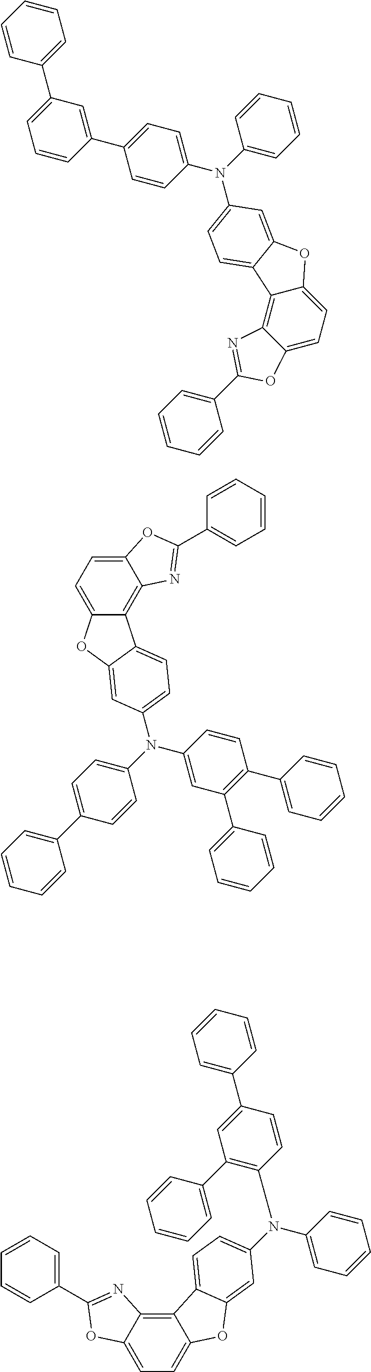

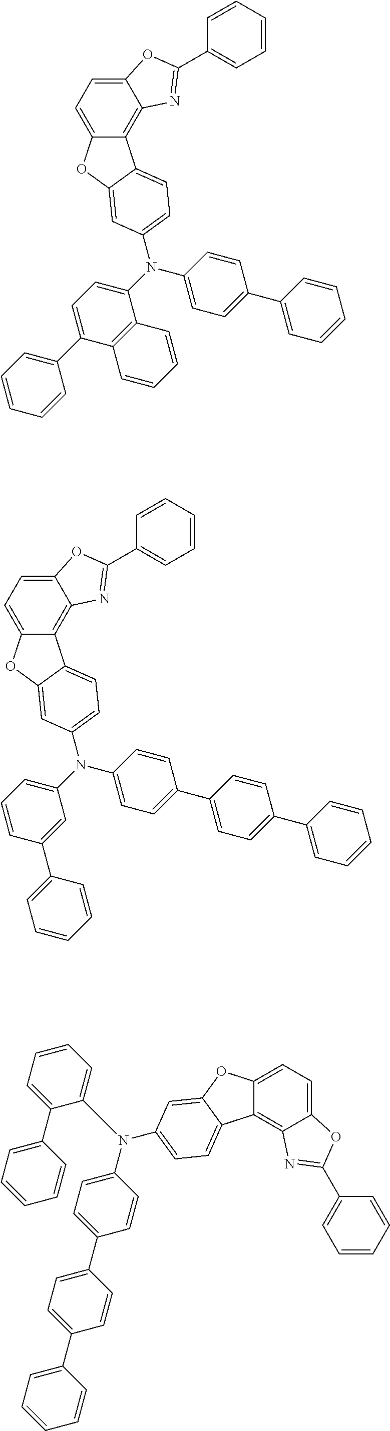 [SMILES: c1ccc(-c2ccc(N(c3ccc4c(c3)oc3ccc5oc(-c6ccccc6)nc5c34)c3ccc(-c4ccccc4)c4ccccc34)cc2)cc1 ; c1ccc(-c2ccc(-c3ccc(N(c4cccc(-c5ccccc5)c4)c4ccc5c(c4)oc4ccc6oc(-c7ccccc7)nc6c45)cc3)cc2)cc1 ; c1ccc(-c2ccc(-c3ccc(N(c4ccc5c(c4)oc4ccc6oc(-c7ccccc7)nc6c45)c4ccccc4-c4ccccc4)cc3)cc2)cc1]

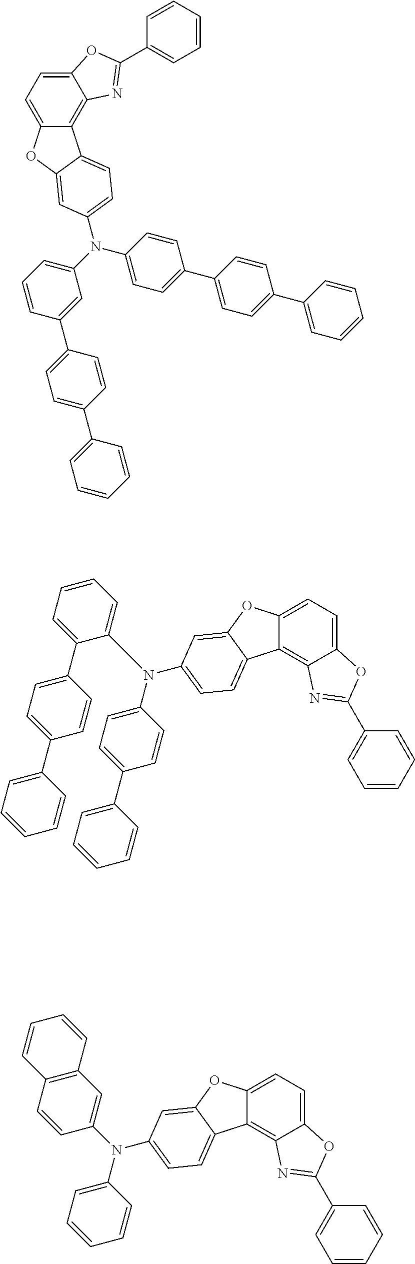 [SMILES: c1ccc(-c2ccc(-c3ccc(N(c4cccc(-c5ccc(-c6ccccc6)cc5)c4)c4ccc5c(c4)oc4ccc6oc(-c7ccccc7)nc6c45)cc3)cc2)cc1 ; c1ccc(-c2ccc(-c3ccccc3N(c3ccc(-c4ccccc4)cc3)c3ccc4c(c3)oc3ccc5oc(-c6ccccc6)nc5c34)cc2)cc1 ; c1ccc(-c2nc3c(ccc4oc5cc(N(c6ccccc6)c6ccc7ccccc7c6)ccc5c43)o2)cc1]

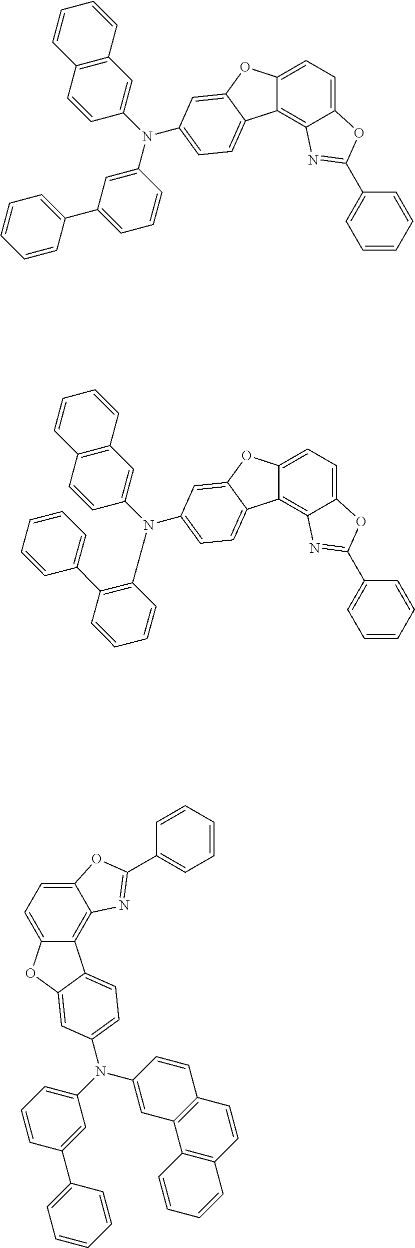 [SMILES: c1ccc(-c2cccc(N(c3ccc4ccccc4c3)c3ccc4c(c3)oc3ccc5oc(-c6ccccc6)nc5c34)c2)cc1 ; c1ccc(-c2nc3c(ccc4oc5cc(N(c6ccc7ccccc7c6)c6ccccc6-c6ccccc6)ccc5c43)o2)cc1 ; c1ccc(-c2cccc(N(c3ccc4c(c3)oc3ccc5oc(-c6ccccc6)nc5c34)c3ccc4ccc5ccccc5c4c3)c2)cc1]

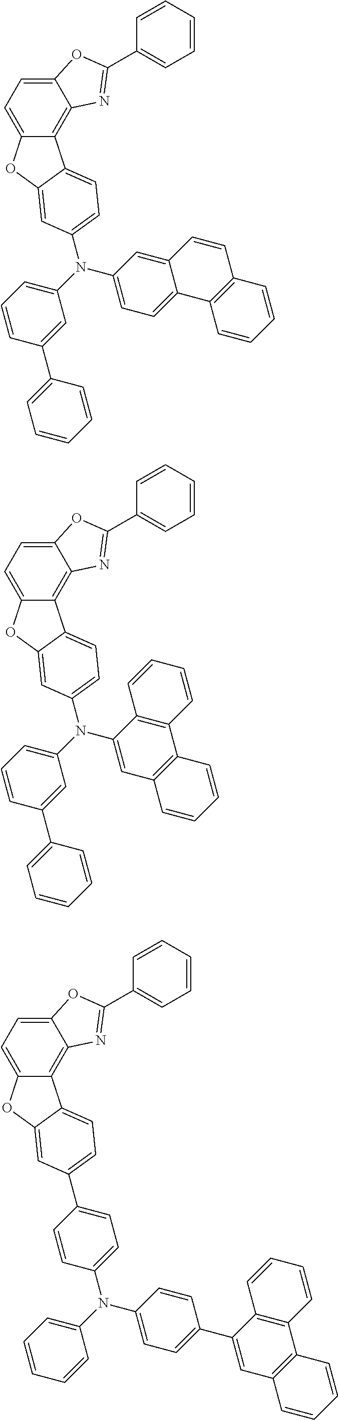 [SMILES: c1ccc(-c2cccc(N(c3ccc4c(ccc5ccccc54)c3)c3ccc4c(c3)oc3ccc5oc(-c6ccccc6)nc5c34)c2)cc1 ; c1ccc(-c2cccc(N(c3ccc4c(c3)oc3ccc5oc(-c6ccccc6)nc5c34)c3cc4ccccc4c4ccccc34)c2)cc1 ; c1ccc(-c2nc3c(ccc4oc5cc(-c6ccc(N(c7ccccc7)c7ccc(-c8cc9ccccc9c9ccccc89)cc7)cc6)ccc5c43)o2)cc1]

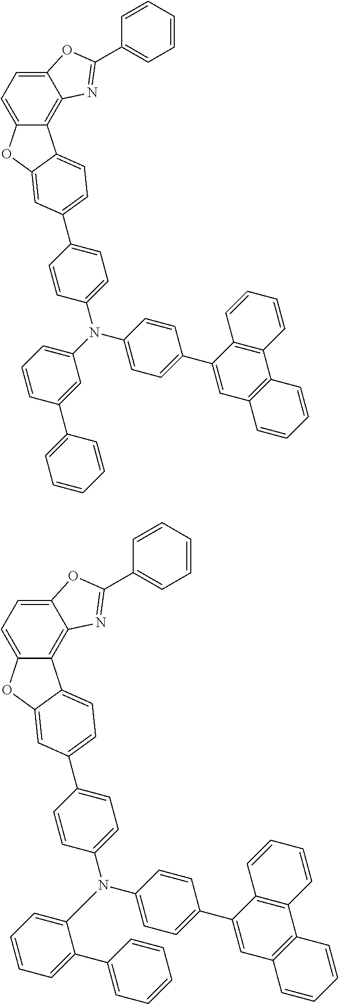 [SMILES: c1ccc(-c2cccc(N(c3ccc(-c4ccc5c(c4)oc4ccc6oc(-c7ccccc7)nc6c45)cc3)c3ccc(-c4cc5ccccc5c5ccccc45)cc3)c2)cc1 ; c1ccc(-c2nc3c(ccc4oc5cc(-c6ccc(N(c7ccc(-c8cc9ccccc9c9ccccc89)cc7)c7ccccc7-c7ccccc7)cc6)ccc5c43)o2)cc1]

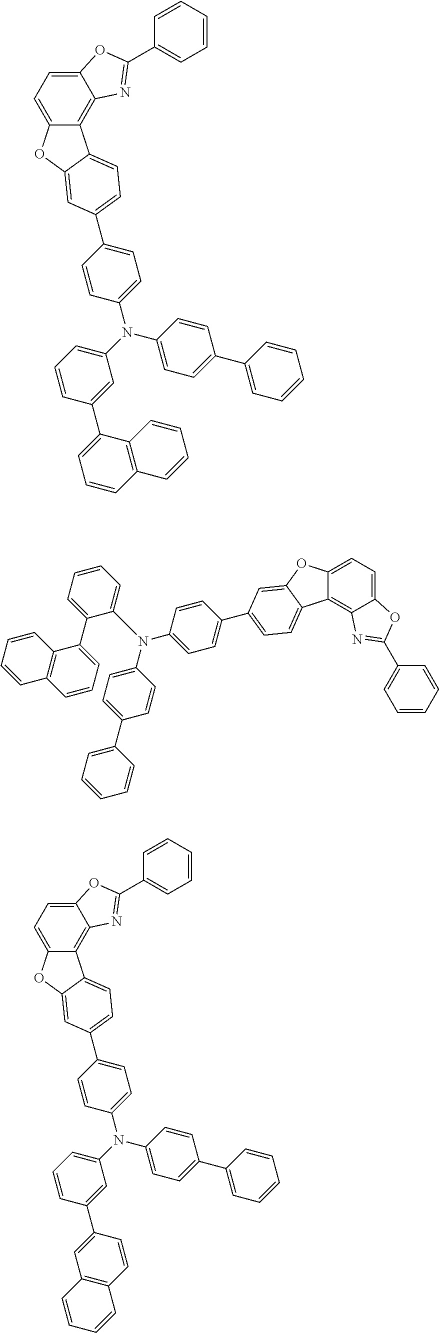 [SMILES: c1ccc(-c2ccc(N(c3ccc(-c4ccc5c(c4)oc4ccc6oc(-c7ccccc7)nc6c45)cc3)c3cccc(-c4cccc5ccccc45)c3)cc2)cc1 ; c1ccc(-c2ccc(N(c3ccc(-c4ccc5c(c4)oc4ccc6oc(-c7ccccc7)nc6c45)cc3)c3ccccc3-c3cccc4ccccc34)cc2)cc1 ; c1ccc(-c2ccc(N(c3ccc(-c4ccc5c(c4)oc4ccc6oc(-c7ccccc7)nc6c45)cc3)c3cccc(-c4ccc5ccccc5c4)c3)cc2)cc1]

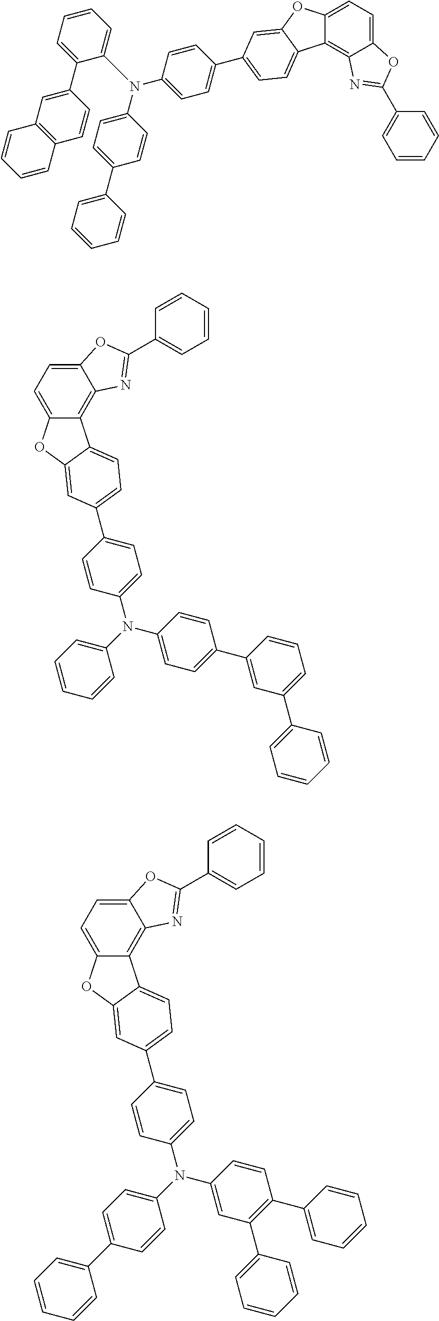 [SMILES: c1ccc(-c2ccc(N(c3ccc(-c4ccc5c(c4)oc4ccc6oc(-c7ccccc7)nc6c45)cc3)c3ccccc3-c3ccc4ccccc4c3)cc2)cc1 ; c1ccc(-c2cccc(-c3ccc(N(c4ccccc4)c4ccc(-c5ccc6c(c5)oc5ccc7oc(-c8ccccc8)nc7c56)cc4)cc3)c2)cc1 ; c1ccc(-c2ccc(N(c3ccc(-c4ccc5c(c4)oc4ccc6oc(-c7ccccc7)nc6c45)cc3)c3ccc(-c4ccccc4)c(-c4ccccc4)c3)cc2)cc1]

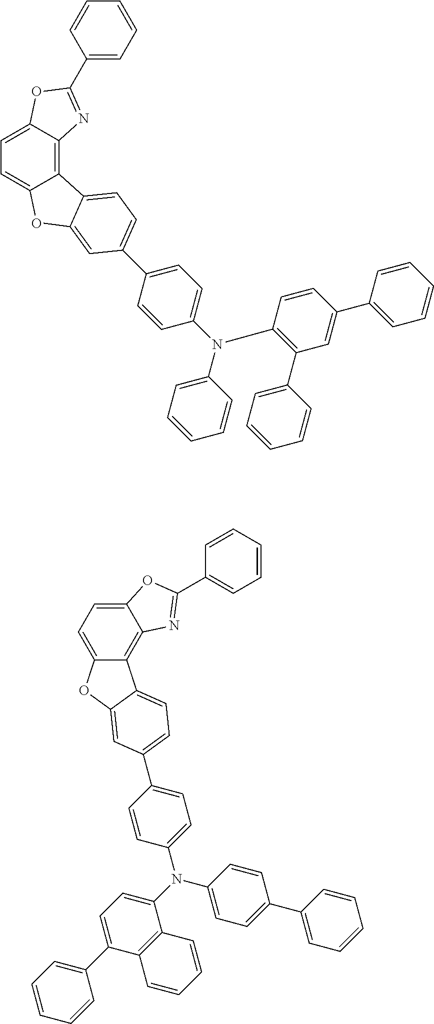 [SMILES: c1ccc(-c2ccc(N(c3ccccc3)c3ccc(-c4ccc5c(c4)oc4ccc6oc(-c7ccccc7)nc6c45)cc3)c(-c3ccccc3)c2)cc1 ; c1ccc(-c2ccc(N(c3ccc(-c4ccc5c(c4)oc4ccc6oc(-c7ccccc7)nc6c45)cc3)c3ccc(-c4ccccc4)c4ccccc34)cc2)cc1]

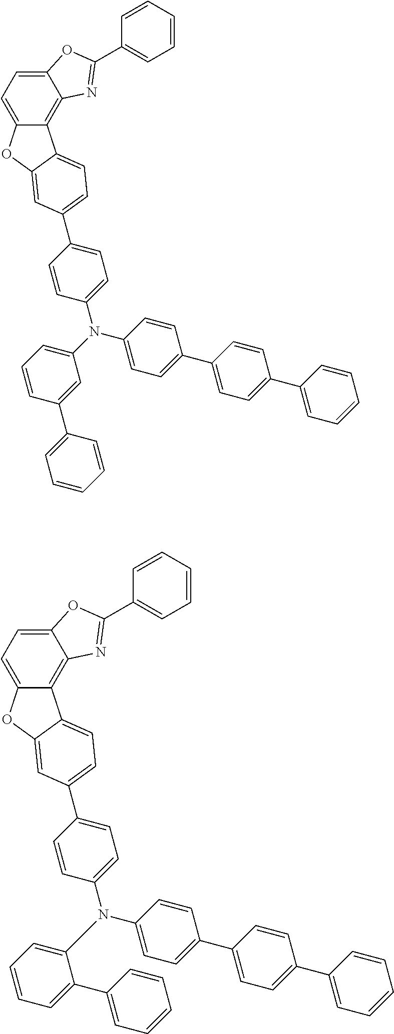 [SMILES: c1ccc(-c2ccc(-c3ccc(N(c4ccc(-c5ccc6c(c5)oc5ccc7oc(-c8ccccc8)nc7c56)cc4)c4cccc(-c5ccccc5)c4)cc3)cc2)cc1 ; c1ccc(-c2ccc(-c3ccc(N(c4ccc(-c5ccc6c(c5)oc5ccc7oc(-c8ccccc8)nc7c56)cc4)c4ccccc4-c4ccccc4)cc3)cc2)cc1]

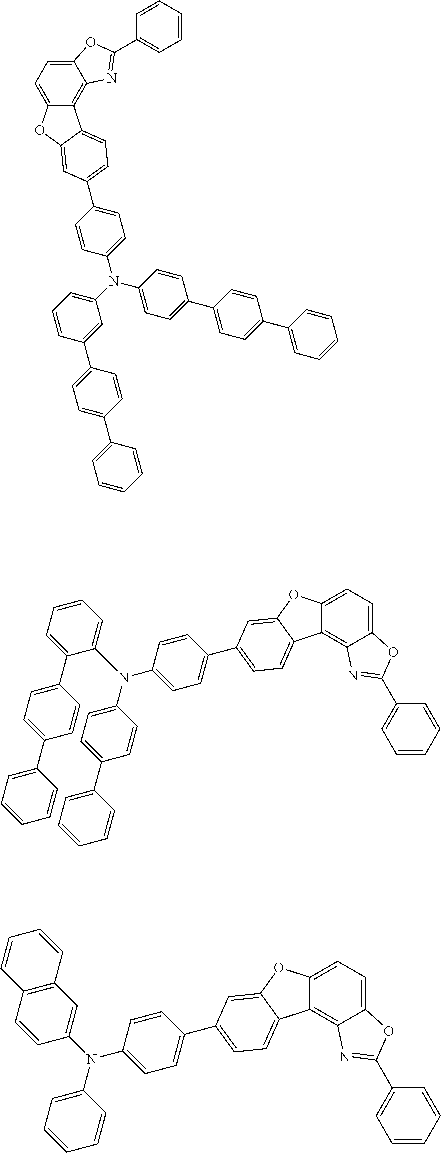 [SMILES: c1ccc(-c2ccc(-c3ccc(N(c4ccc(-c5ccc6c(c5)oc5ccc7oc(-c8ccccc8)nc7c56)cc4)c4cccc(-c5ccc(-c6ccccc6)cc5)c4)cc3)cc2)cc1 ; c1ccc(-c2ccc(-c3ccccc3N(c3ccc(-c4ccccc4)cc3)c3ccc(-c4ccc5c(c4)oc4ccc6oc(-c7ccccc7)nc6c45)cc3)cc2)cc1 ; c1ccc(-c2nc3c(ccc4oc5cc(-c6ccc(N(c7ccccc7)c7ccc8ccccc8c7)cc6)ccc5c43)o2)cc1]

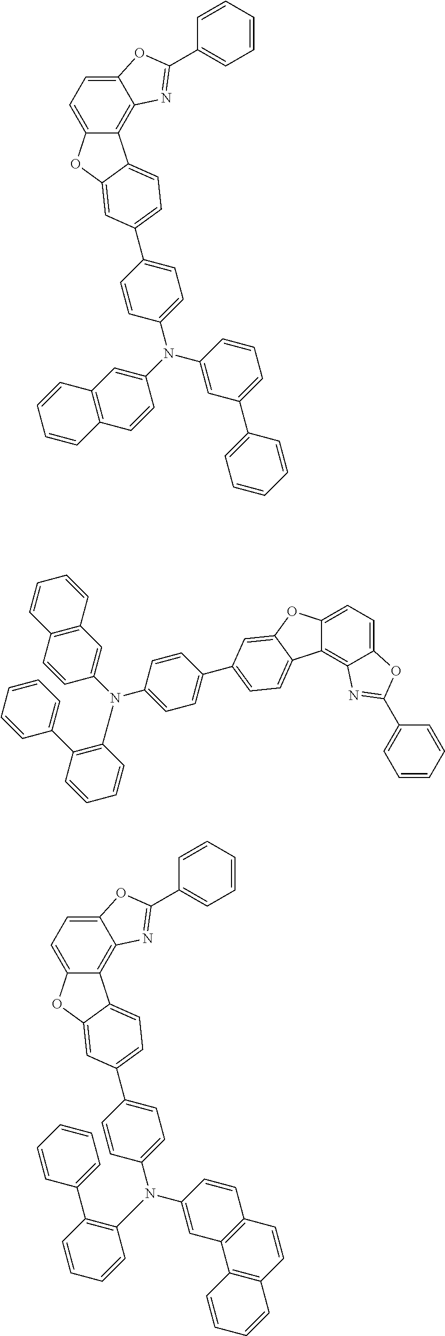 [SMILES: c1ccc(-c2cccc(N(c3ccc(-c4ccc5c(c4)oc4ccc6oc(-c7ccccc7)nc6c45)cc3)c3ccc4ccccc4c3)c2)cc1 ; c1ccc(-c2nc3c(ccc4oc5cc(-c6ccc(N(c7ccc8ccccc8c7)c7ccccc7-c7ccccc7)cc6)ccc5c43)o2)cc1 ; c1ccc(-c2nc3c(ccc4oc5cc(-c6ccc(N(c7ccc8ccc9ccccc9c8c7)c7ccccc7-c7ccccc7)cc6)ccc5c43)o2)cc1]

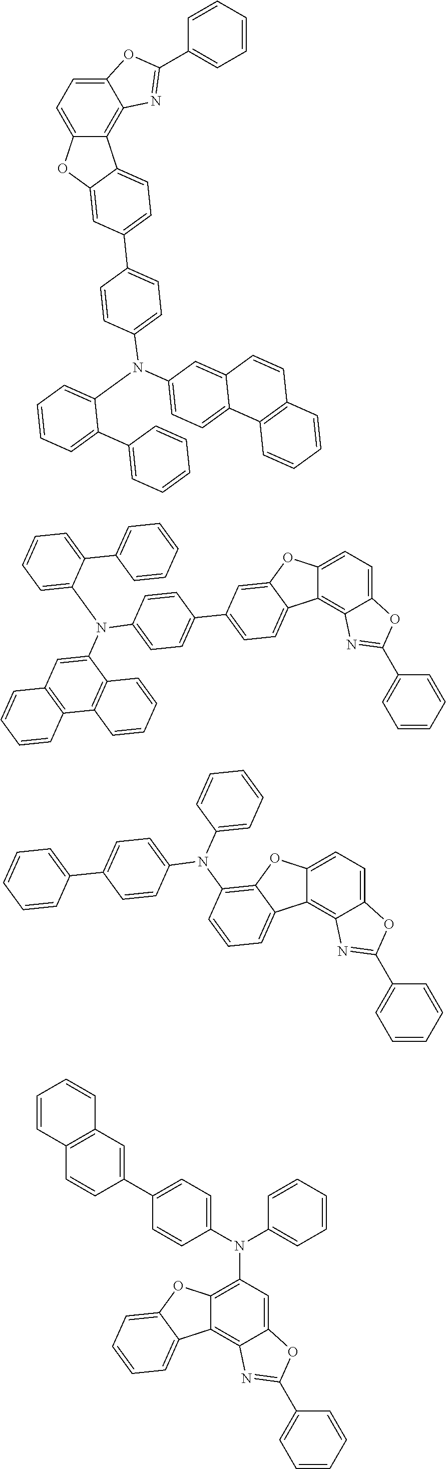

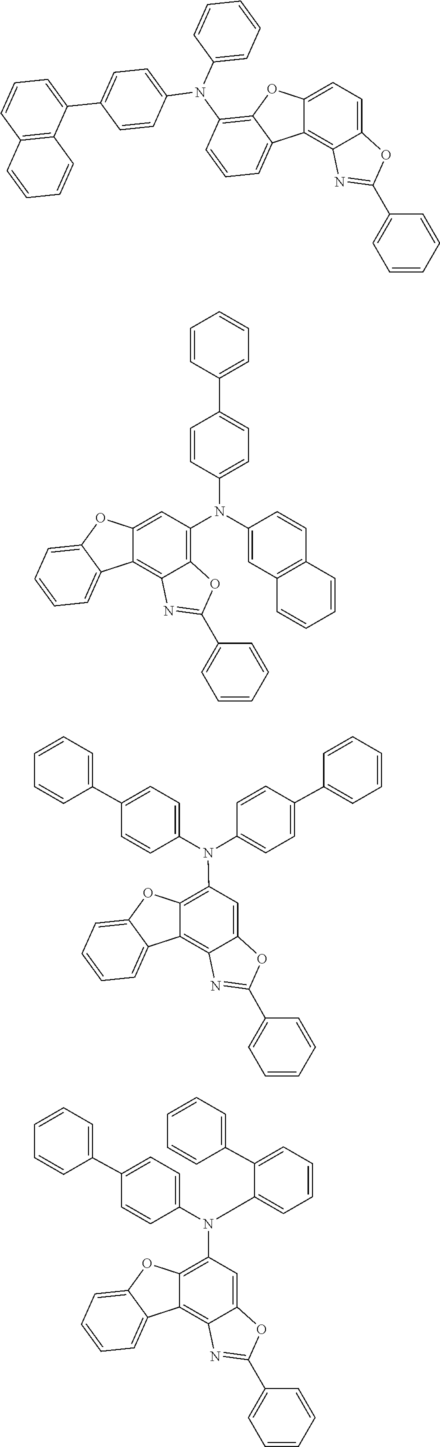 [SMILES: c1ccc(-c2nc3c(ccc4oc5c(N(c6ccccc6)c6ccc(-c7cccc8ccccc78)cc6)cccc5c43)o2)cc1 ; c1ccc(-c2ccc(N(c3ccc4ccccc4c3)c3cc4oc5ccccc5c4c4nc(-c5ccccc5)oc34)cc2)cc1 ; c1ccc(-c2ccc(N(c3ccc(-c4ccccc4)cc3)c3cc4oc(-c5ccccc5)nc4c4c3oc3ccccc34)cc2)cc1 ; c1ccc(-c2ccc(N(c3ccccc3-c3ccccc3)c3cc4oc(-c5ccccc5)nc4c4c3oc3ccccc34)cc2)cc1]

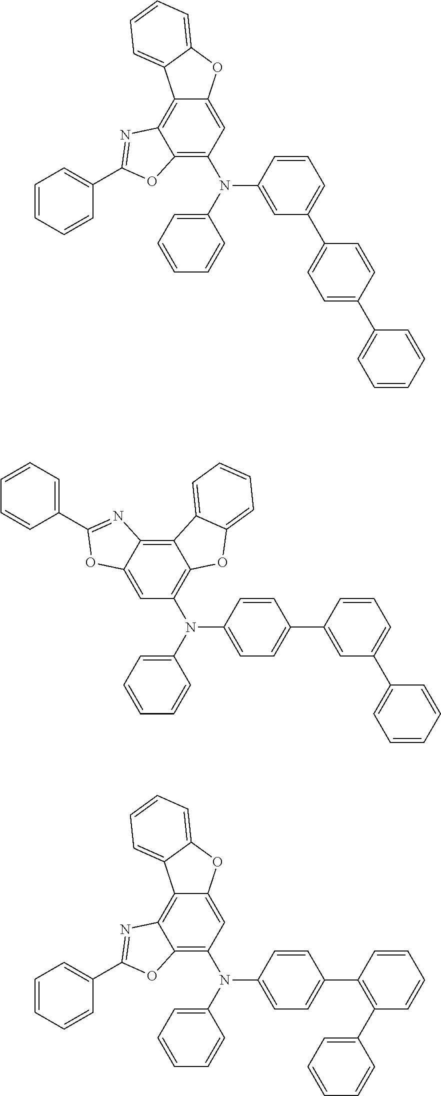 [SMILES: c1ccc(-c2ccc(-c3cccc(N(c4ccccc4)c4cc5oc6ccccc6c5c5nc(-c6ccccc6)oc45)c3)cc2)cc1 ; c1ccc(-c2cccc(-c3ccc(N(c4ccccc4)c4cc5oc(-c6ccccc6)nc5c5c4oc4ccccc45)cc3)c2)cc1 ; c1ccc(-c2nc3c(o2)c(N(c2ccccc2)c2ccc(-c4ccccc4-c4ccccc4)cc2)cc2oc4ccccc4c23)cc1]

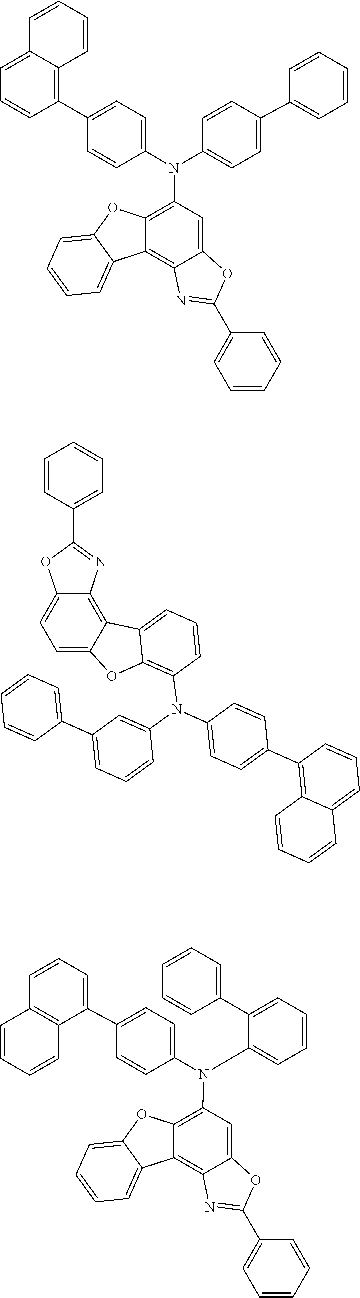 [SMILES: c1ccc(-c2ccc(N(c3ccc(-c4cccc5ccccc45)cc3)c3cc4oc(-c5ccccc5)nc4c4c3oc3ccccc34)cc2)cc1 ; c1ccc(-c2cccc(N(c3ccc(-c4cccc5ccccc45)cc3)c3cccc4c3oc3ccc5oc(-c6ccccc6)nc5c34)c2)cc1 ; c1ccc(-c2nc3c(cc(N(c4ccc(-c5cccc6ccccc56)cc4)c4ccccc4-c4ccccc4)c4oc5ccccc5c43)o2)cc1]

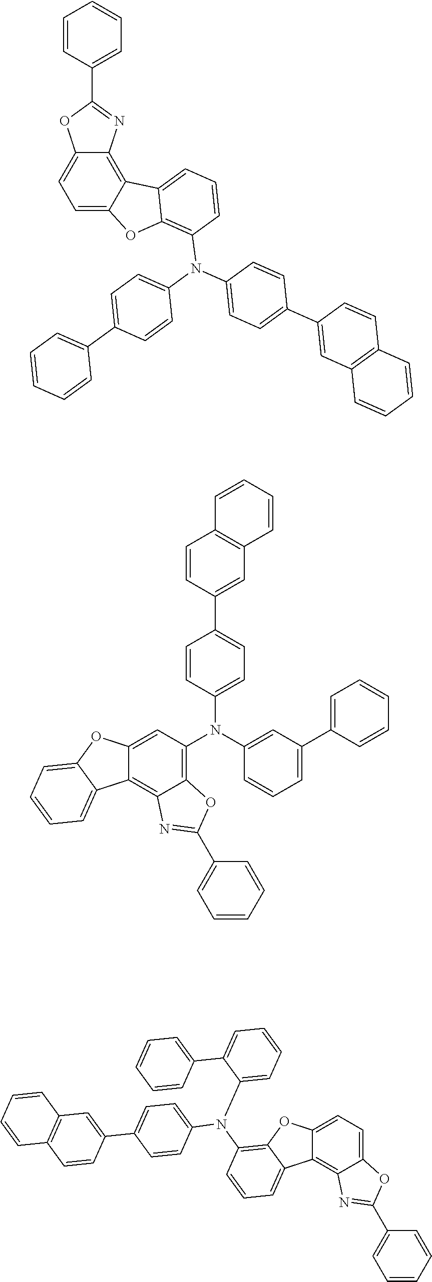 [SMILES: c1ccc(-c2ccc(N(c3ccc(-c4ccc5ccccc5c4)cc3)c3cccc4c3oc3ccc5oc(-c6ccccc6)nc5c34)cc2)cc1 ; c1ccc(-c2cccc(N(c3ccc(-c4ccc5ccccc5c4)cc3)c3cc4oc5ccccc5c4c4nc(-c5ccccc5)oc34)c2)cc1 ; c1ccc(-c2nc3c(ccc4oc5c(N(c6ccc(-c7ccc8ccccc8c7)cc6)c6ccccc6-c6ccccc6)cccc5c43)o2)cc1]

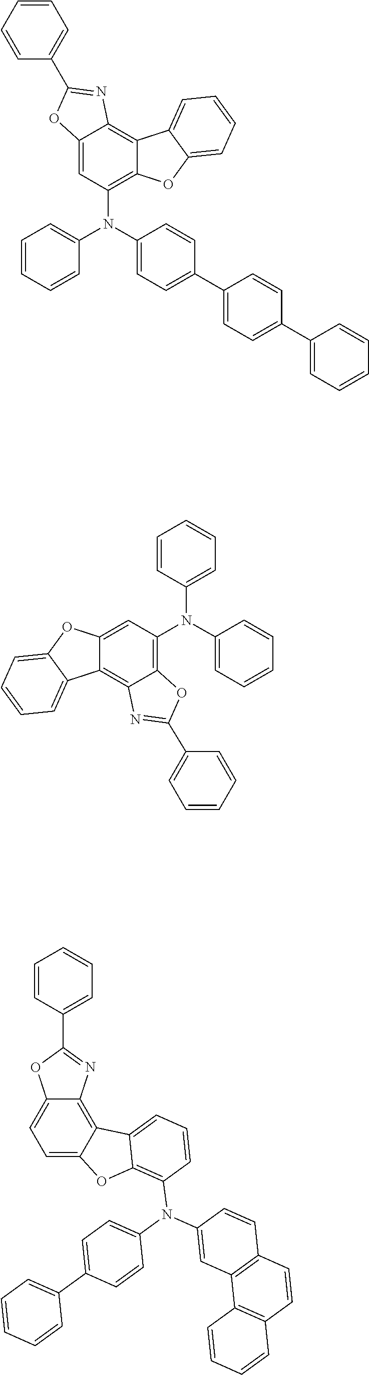 [SMILES: c1ccc(-c2ccc(-c3ccc(N(c4ccccc4)c4cc5oc(-c6ccccc6)nc5c5c4oc4ccccc45)cc3)cc2)cc1 ; c1ccc(-c2nc3c(o2)c(N(c2ccccc2)c2ccccc2)cc2oc4ccccc4c23)cc1 ; c1ccc(-c2ccc(N(c3ccc4ccc5ccccc5c4c3)c3cccc4c3oc3ccc5oc(-c6ccccc6)nc5c34)cc2)cc1]

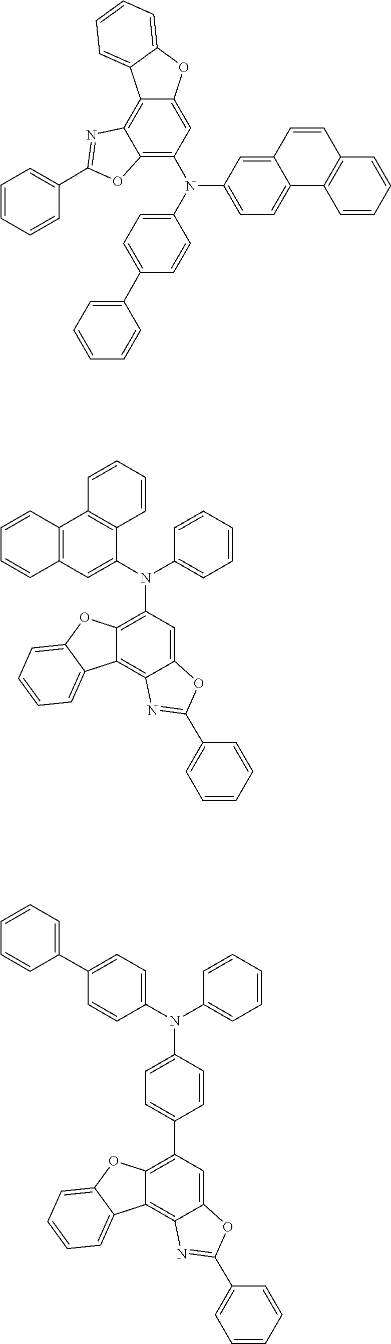 [SMILES: c1ccc(-c2ccc(N(c3ccc4c(ccc5ccccc54)c3)c3cc4oc5ccccc5c4c4nc(-c5ccccc5)oc34)cc2)cc1 ; c1ccc(-c2nc3c(cc(N(c4ccccc4)c4cc5ccccc5c5ccccc45)c4oc5ccccc5c43)o2)cc1 ; c1ccc(-c2ccc(N(c3ccccc3)c3ccc(-c4cc5oc(-c6ccccc6)nc5c5c4oc4ccccc45)cc3)cc2)cc1]

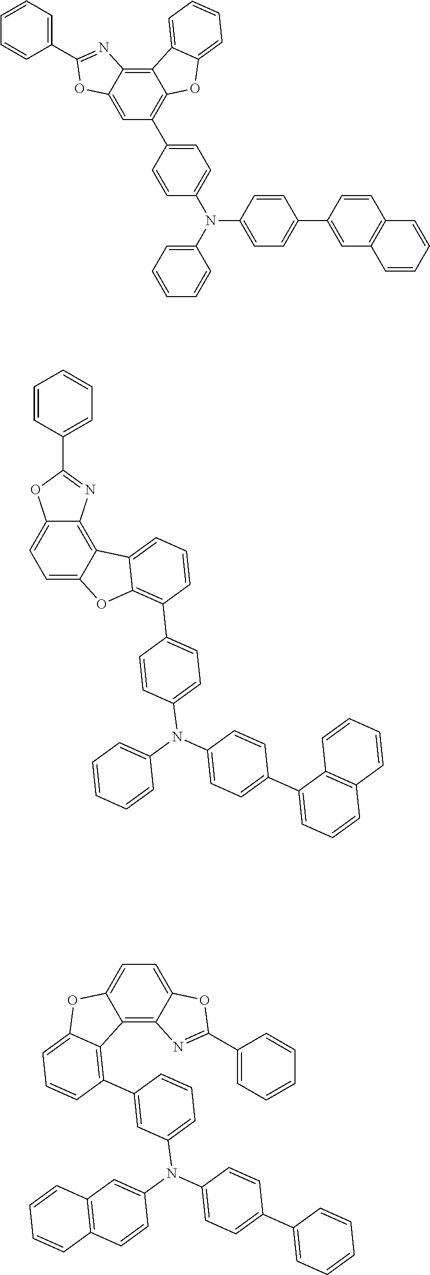 [SMILES: c1ccc(-c2nc3c(cc(-c4ccc(N(c5ccccc5)c5ccc(-c6ccc7ccccc7c6)cc5)cc4)c4oc5ccccc5c43)o2)cc1 ; c1ccc(-c2nc3c(ccc4oc5c(-c6ccc(N(c7ccccc7)c7ccc(-c8cccc9ccccc89)cc7)cc6)cccc5c43)o2)cc1 ; c1ccc(-c2ccc(N(c3cccc(-c4cccc5oc6ccc7oc(-c8ccccc8)nc7c6c45)c3)c3ccc4ccccc4c3)cc2)cc1]

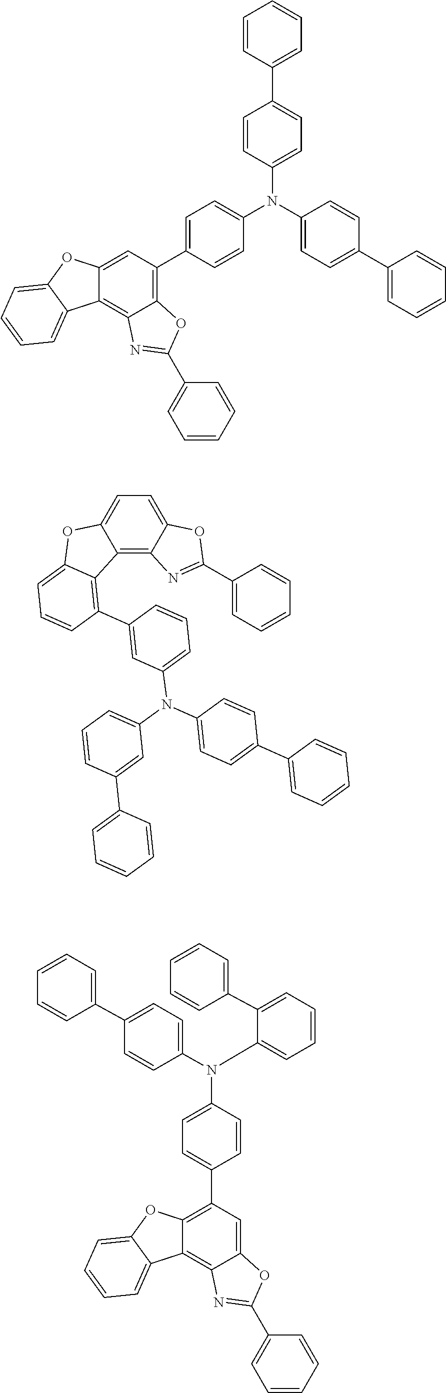 [SMILES: c1ccc(-c2ccc(N(c3ccc(-c4ccccc4)cc3)c3ccc(-c4cc5oc6ccccc6c5c5nc(-c6ccccc6)oc45)cc3)cc2)cc1 ; c1ccc(-c2ccc(N(c3cccc(-c4ccccc4)c3)c3cccc(-c4cccc5oc6ccc7oc(-c8ccccc8)nc7c6c45)c3)cc2)cc1 ; c1ccc(-c2ccc(N(c3ccc(-c4cc5oc(-c6ccccc6)nc5c5c4oc4ccccc45)cc3)c3ccccc3-c3ccccc3)cc2)cc1]

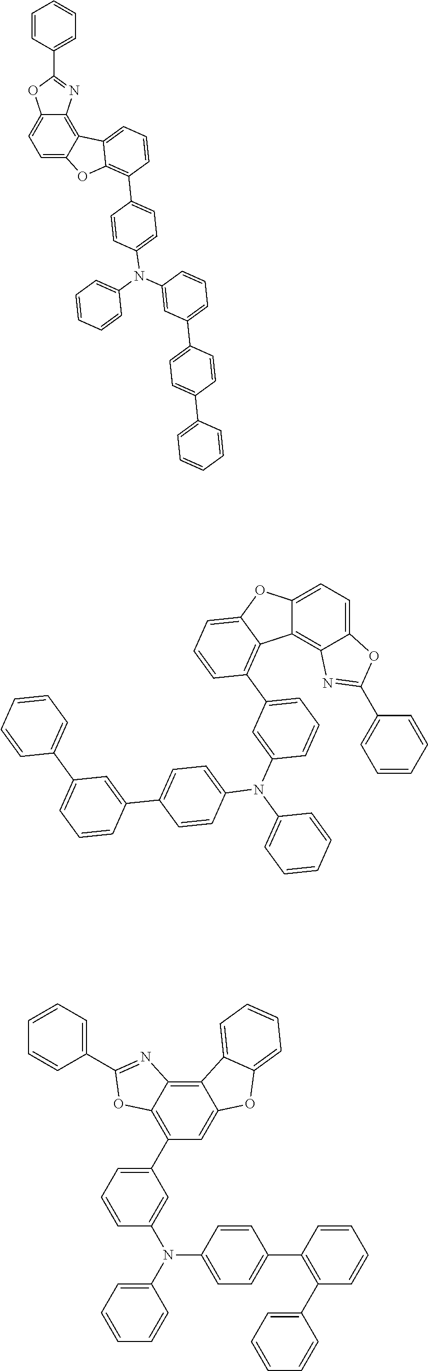 [SMILES: c1ccc(-c2ccc(-c3cccc(N(c4ccccc4)c4ccc(-c5cccc6c5oc5ccc7oc(-c8ccccc8)nc7c56)cc4)c3)cc2)cc1 ; c1ccc(-c2cccc(-c3ccc(N(c4ccccc4)c4cccc(-c5cccc6oc7ccc8oc(-c9ccccc9)nc8c7c56)c4)cc3)c2)cc1 ; c1ccc(-c2nc3c(o2)c(-c2cccc(N(c4ccccc4)c4ccc(-c5ccccc5-c5ccccc5)cc4)c2)cc2oc4ccccc4c23)cc1]

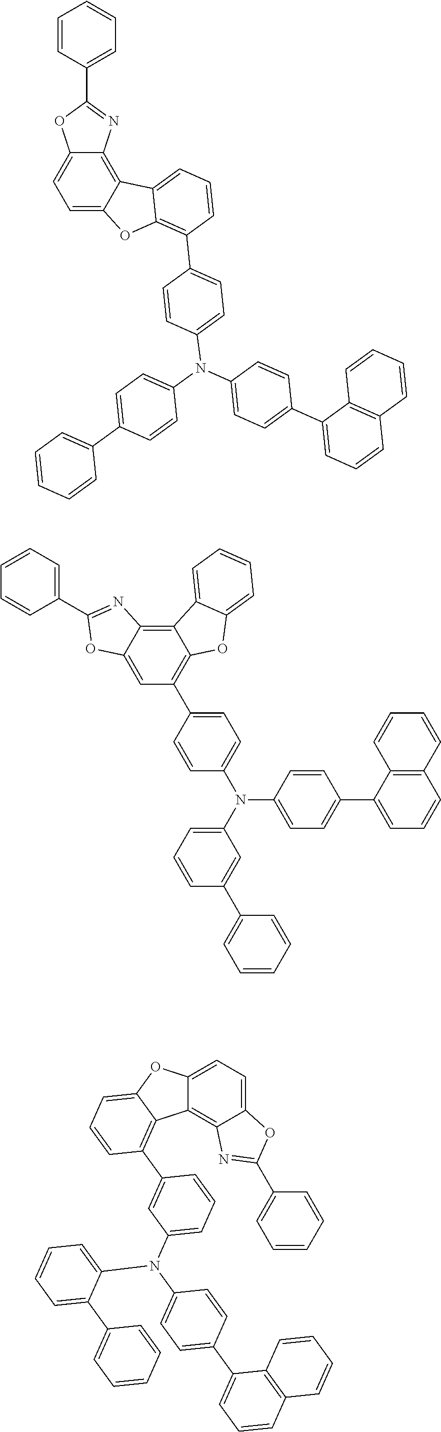 [SMILES: c1ccc(-c2ccc(N(c3ccc(-c4cccc5ccccc45)cc3)c3ccc(-c4cccc5c4oc4ccc6oc(-c7ccccc7)nc6c45)cc3)cc2)cc1 ; c1ccc(-c2cccc(N(c3ccc(-c4cccc5ccccc45)cc3)c3ccc(-c4cc5oc(-c6ccccc6)nc5c5c4oc4ccccc45)cc3)c2)cc1 ; c1ccc(-c2nc3c(ccc4oc5cccc(-c6cccc(N(c7ccc(-c8cccc9ccccc89)cc7)c7ccccc7-c7ccccc7)c6)c5c43)o2)cc1]

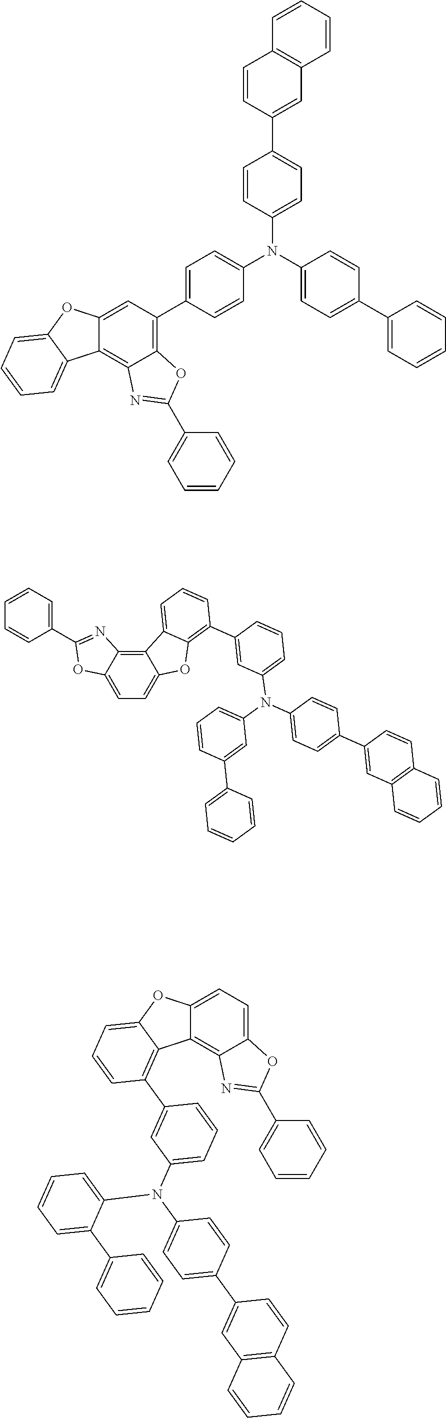 [SMILES: c1ccc(-c2ccc(N(c3ccc(-c4ccc5ccccc5c4)cc3)c3ccc(-c4cc5oc6ccccc6c5c5nc(-c6ccccc6)oc45)cc3)cc2)cc1 ; c1ccc(-c2cccc(N(c3ccc(-c4ccc5ccccc5c4)cc3)c3cccc(-c4cccc5c4oc4ccc6oc(-c7ccccc7)nc6c45)c3)c2)cc1 ; c1ccc(-c2nc3c(ccc4oc5cccc(-c6cccc(N(c7ccc(-c8ccc9ccccc9c8)cc7)c7ccccc7-c7ccccc7)c6)c5c43)o2)cc1]

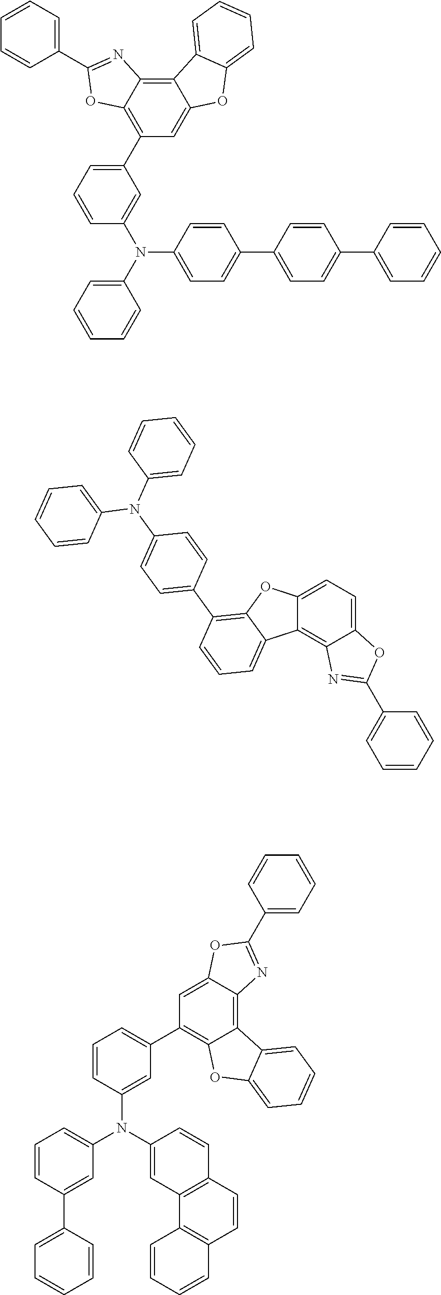 [SMILES: c1ccc(-c2ccc(-c3ccc(N(c4ccccc4)c4cccc(-c5cc6oc7ccccc7c6c6nc(-c7ccccc7)oc56)c4)cc3)cc2)cc1 ; c1ccc(-c2nc3c(ccc4oc5c(-c6ccc(N(c7ccccc7)c7ccccc7)cc6)cccc5c43)o2)cc1 ; c1ccc(-c2cccc(N(c3cccc(-c4cc5oc(-c6ccccc6)nc5c5c4oc4ccccc45)c3)c3ccc4ccc5ccccc5c4c3)c2)cc1]

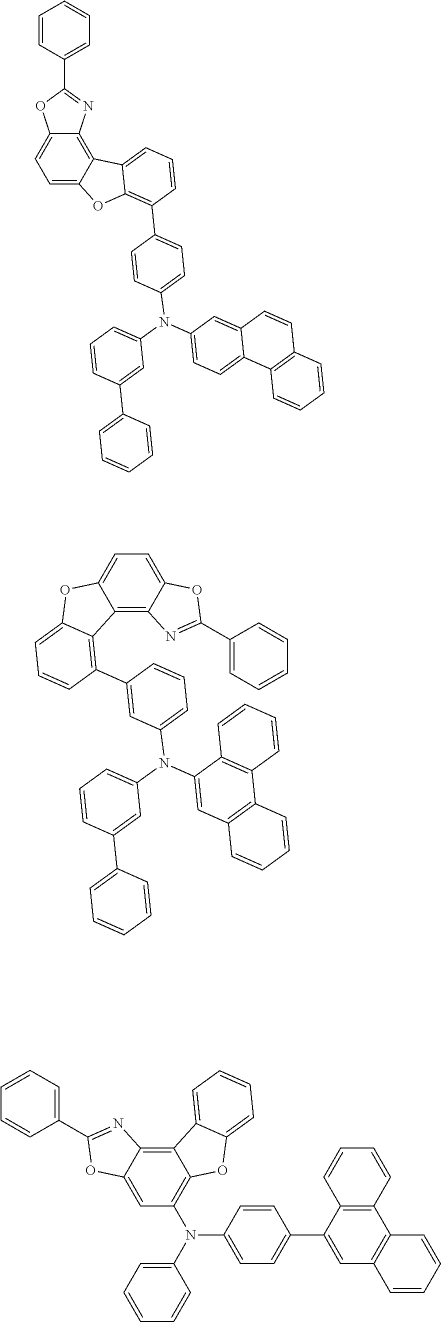 [SMILES: c1ccc(-c2cccc(N(c3ccc(-c4cccc5c4oc4ccc6oc(-c7ccccc7)nc6c45)cc3)c3ccc4c(ccc5ccccc54)c3)c2)cc1 ; c1ccc(-c2cccc(N(c3cccc(-c4cccc5oc6ccc7oc(-c8ccccc8)nc7c6c45)c3)c3cc4ccccc4c4ccccc34)c2)cc1 ; c1ccc(-c2nc3c(cc(N(c4ccccc4)c4ccc(-c5cc6ccccc6c6ccccc56)cc4)c4oc5ccccc5c43)o2)cc1]

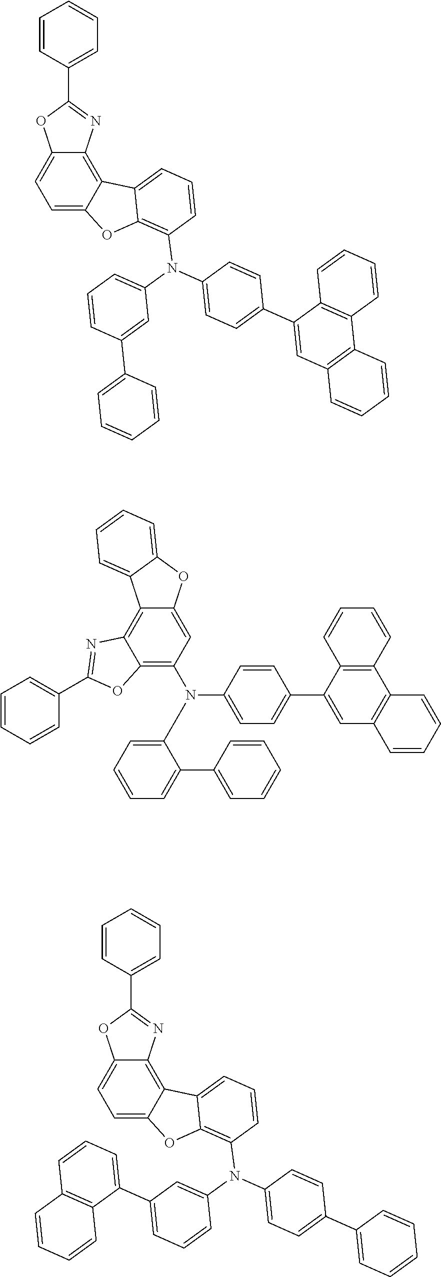 [SMILES: c1ccc(-c2cccc(N(c3ccc(-c4cc5ccccc5c5ccccc45)cc3)c3cccc4c3oc3ccc5oc(-c6ccccc6)nc5c34)c2)cc1 ; c1ccc(-c2nc3c(o2)c(N(c2ccc(-c4cc5ccccc5c5ccccc45)cc2)c2ccccc2-c2ccccc2)cc2oc4ccccc4c23)cc1 ; c1ccc(-c2ccc(N(c3cccc(-c4cccc5ccccc45)c3)c3cccc4c3oc3ccc5oc(-c6ccccc6)nc5c34)cc2)cc1]

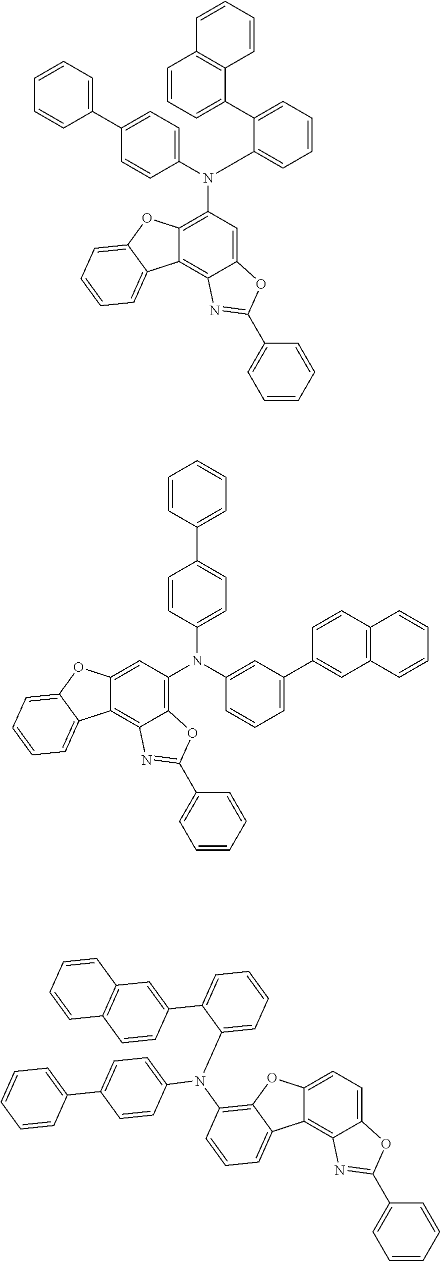 [SMILES: c1ccc(-c2ccc(N(c3ccccc3-c3cccc4ccccc34)c3cc4oc(-c5ccccc5)nc4c4c3oc3ccccc34)cc2)cc1 ; c1ccc(-c2ccc(N(c3cccc(-c4ccc5ccccc5c4)c3)c3cc4oc5ccccc5c4c4nc(-c5ccccc5)oc34)cc2)cc1 ; c1ccc(-c2ccc(N(c3ccccc3-c3ccc4ccccc4c3)c3cccc4c3oc3ccc5oc(-c6ccccc6)nc5c34)cc2)cc1]

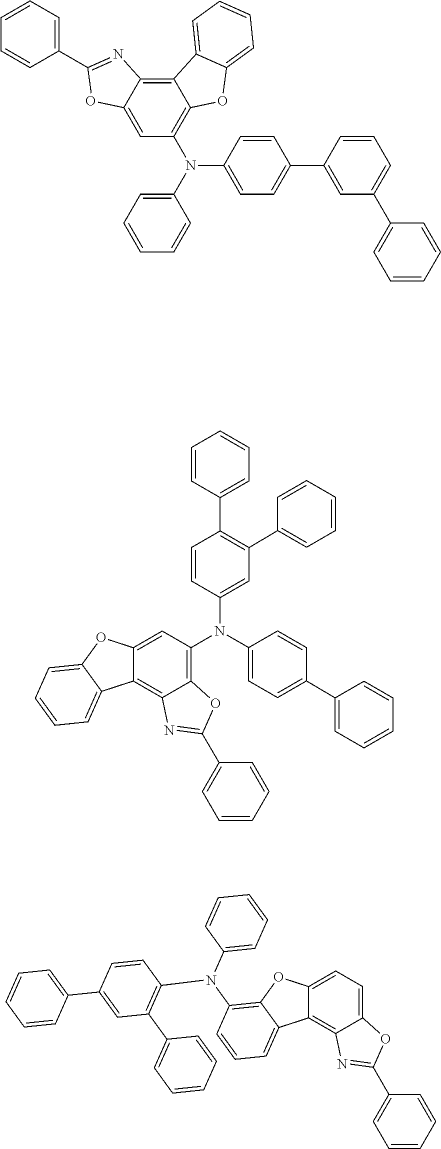 [SMILES: c1ccc(-c2cccc(-c3ccc(N(c4ccccc4)c4cc5oc(-c6ccccc6)nc5c5c4oc4ccccc45)cc3)c2)cc1 ; c1ccc(-c2ccc(N(c3ccc(-c4ccccc4)c(-c4ccccc4)c3)c3cc4oc5ccccc5c4c4nc(-c5ccccc5)oc34)cc2)cc1 ; c1ccc(-c2ccc(N(c3ccccc3)c3cccc4c3oc3ccc5oc(-c6ccccc6)nc5c34)c(-c3ccccc3)c2)cc1]

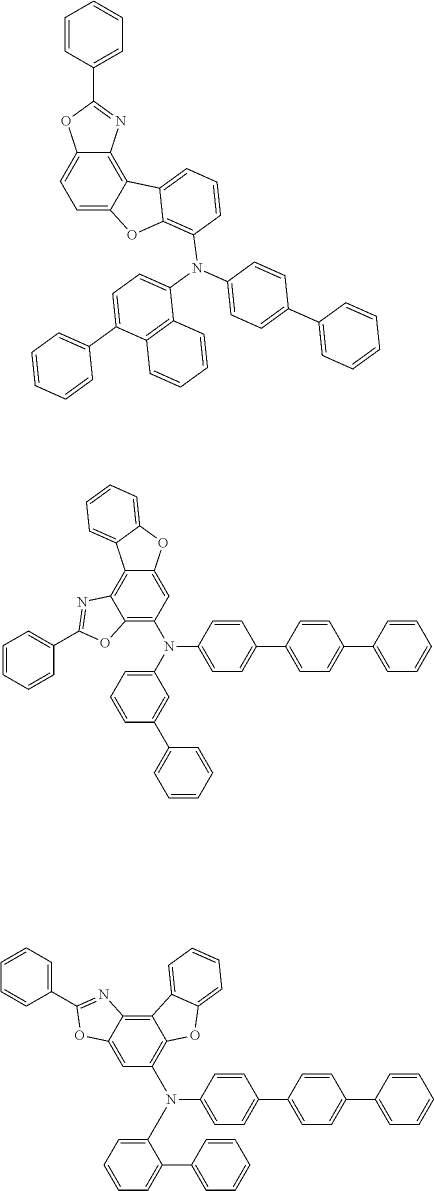 [SMILES: c1ccc(-c2ccc(N(c3ccc(-c4ccccc4)c4ccccc34)c3cccc4c3oc3ccc5oc(-c6ccccc6)nc5c34)cc2)cc1 ; c1ccc(-c2ccc(-c3ccc(N(c4cccc(-c5ccccc5)c4)c4cc5oc6ccccc6c5c5nc(-c6ccccc6)oc45)cc3)cc2)cc1 ; c1ccc(-c2ccc(-c3ccc(N(c4ccccc4-c4ccccc4)c4cc5oc(-c6ccccc6)nc5c5c4oc4ccccc45)cc3)cc2)cc1]

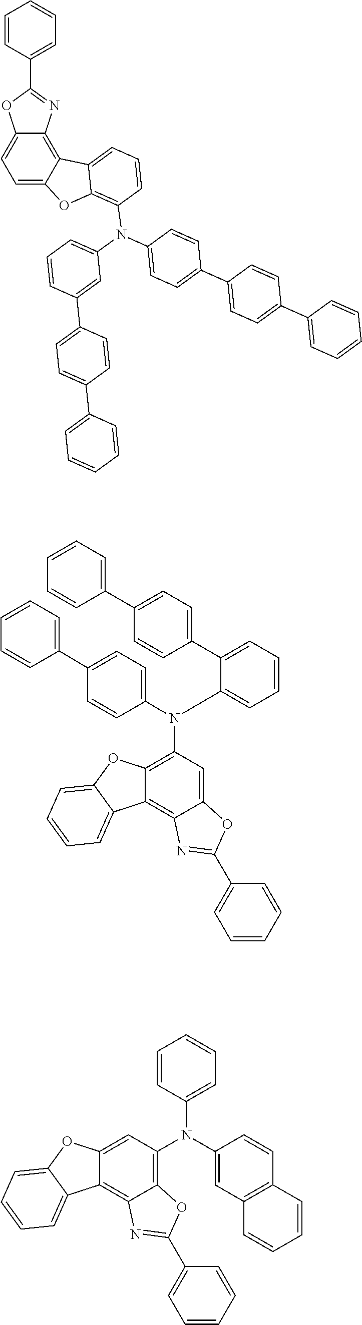 [SMILES: c1ccc(-c2ccc(-c3ccc(N(c4cccc(-c5ccc(-c6ccccc6)cc5)c4)c4cccc5c4oc4ccc6oc(-c7ccccc7)nc6c45)cc3)cc2)cc1 ; c1ccc(-c2ccc(-c3ccccc3N(c3ccc(-c4ccccc4)cc3)c3cc4oc(-c5ccccc5)nc4c4c3oc3ccccc34)cc2)cc1 ; c1ccc(-c2nc3c(o2)c(N(c2ccccc2)c2ccc4ccccc4c2)cc2oc4ccccc4c23)cc1]

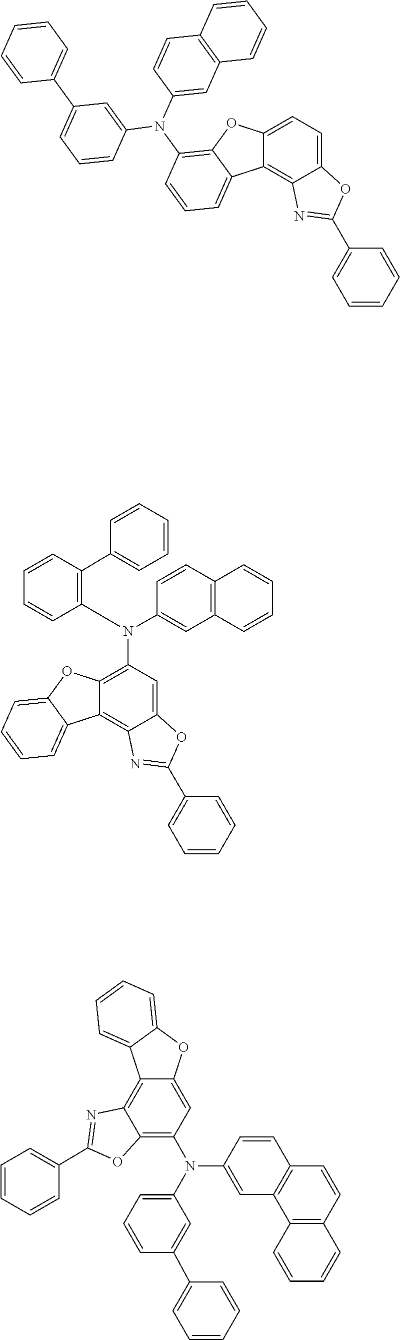 [SMILES: c1ccc(-c2cccc(N(c3ccc4ccccc4c3)c3cccc4c3oc3ccc5oc(-c6ccccc6)nc5c34)c2)cc1 ; c1ccc(-c2nc3c(cc(N(c4ccc5ccccc5c4)c4ccccc4-c4ccccc4)c4oc5ccccc5c43)o2)cc1 ; c1ccc(-c2cccc(N(c3ccc4ccc5ccccc5c4c3)c3cc4oc5ccccc5c4c4nc(-c5ccccc5)oc34)c2)cc1]

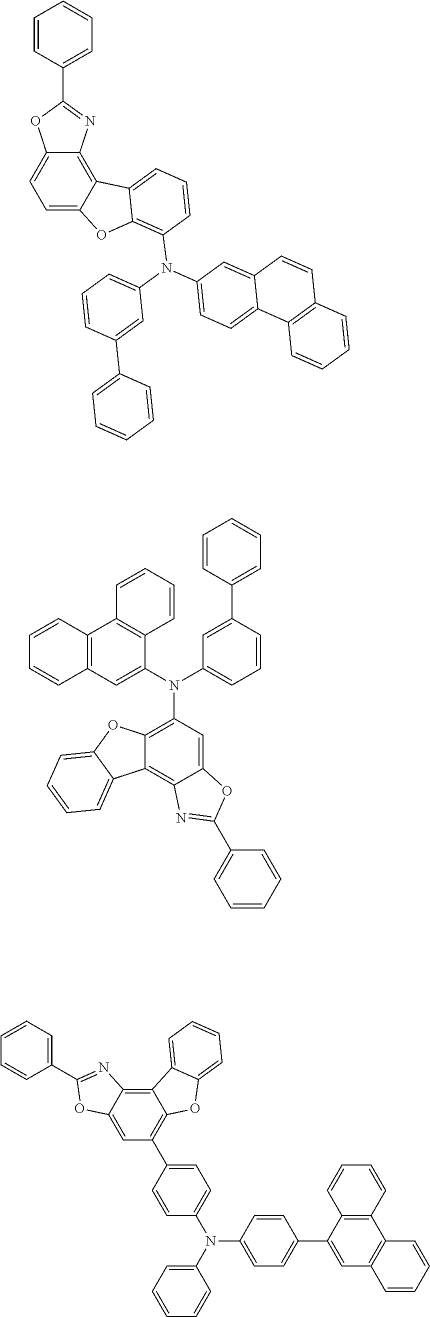 [SMILES: c1ccc(-c2cccc(N(c3ccc4c(ccc5ccccc54)c3)c3cccc4c3oc3ccc5oc(-c6ccccc6)nc5c34)c2)cc1 ; c1ccc(-c2cccc(N(c3cc4ccccc4c4ccccc34)c3cc4oc(-c5ccccc5)nc4c4c3oc3ccccc34)c2)cc1 ; c1ccc(-c2nc3c(cc(-c4ccc(N(c5ccccc5)c5ccc(-c6cc7ccccc7c7ccccc67)cc5)cc4)c4oc5ccccc5c43)o2)cc1]

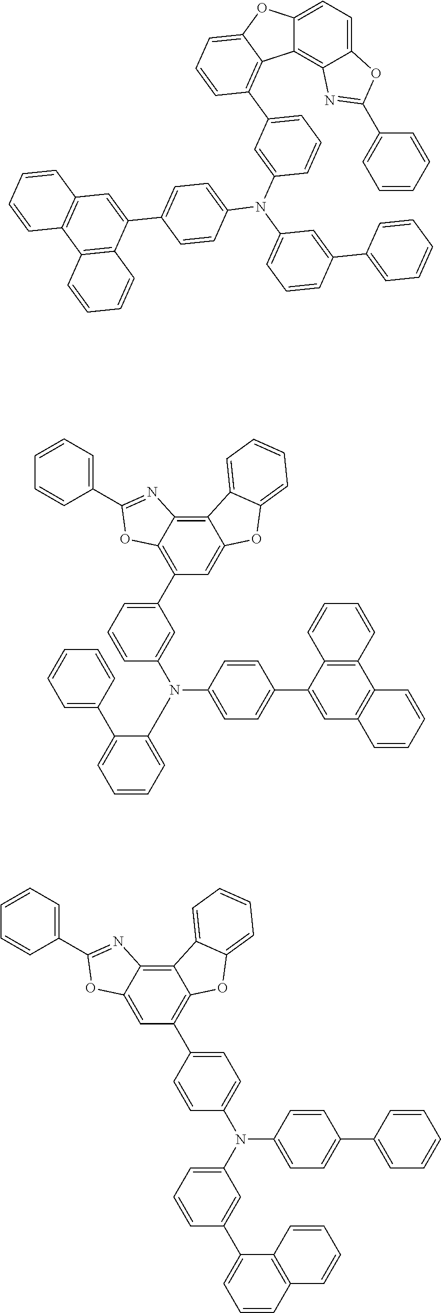 [SMILES: c1ccc(-c2cccc(N(c3ccc(-c4cc5ccccc5c5ccccc45)cc3)c3cccc(-c4cccc5oc6ccc7oc(-c8ccccc8)nc7c6c45)c3)c2)cc1 ; c1ccc(-c2nc3c(o2)c(-c2cccc(N(c4ccc(-c5cc6ccccc6c6ccccc56)cc4)c4ccccc4-c4ccccc4)c2)cc2oc4ccccc4c23)cc1 ; c1ccc(-c2ccc(N(c3ccc(-c4cc5oc(-c6ccccc6)nc5c5c4oc4ccccc45)cc3)c3cccc(-c4cccc5ccccc45)c3)cc2)cc1]

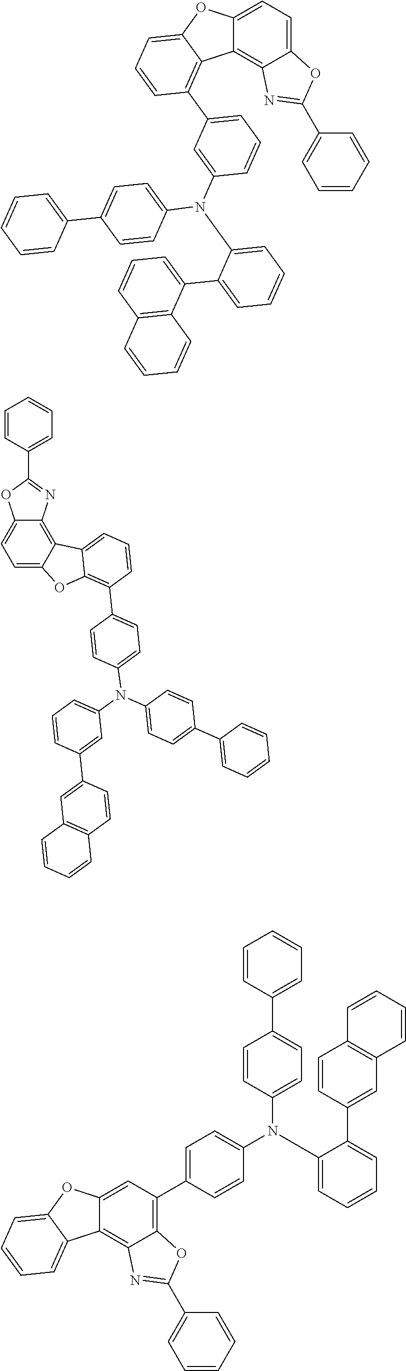 [SMILES: c1ccc(-c2ccc(N(c3cccc(-c4cccc5oc6ccc7oc(-c8ccccc8)nc7c6c45)c3)c3ccccc3-c3cccc4ccccc34)cc2)cc1 ; c1ccc(-c2ccc(N(c3ccc(-c4cccc5c4oc4ccc6oc(-c7ccccc7)nc6c45)cc3)c3cccc(-c4ccc5ccccc5c4)c3)cc2)cc1 ; c1ccc(-c2ccc(N(c3ccc(-c4cc5oc6ccccc6c5c5nc(-c6ccccc6)oc45)cc3)c3ccccc3-c3ccc4ccccc4c3)cc2)cc1]

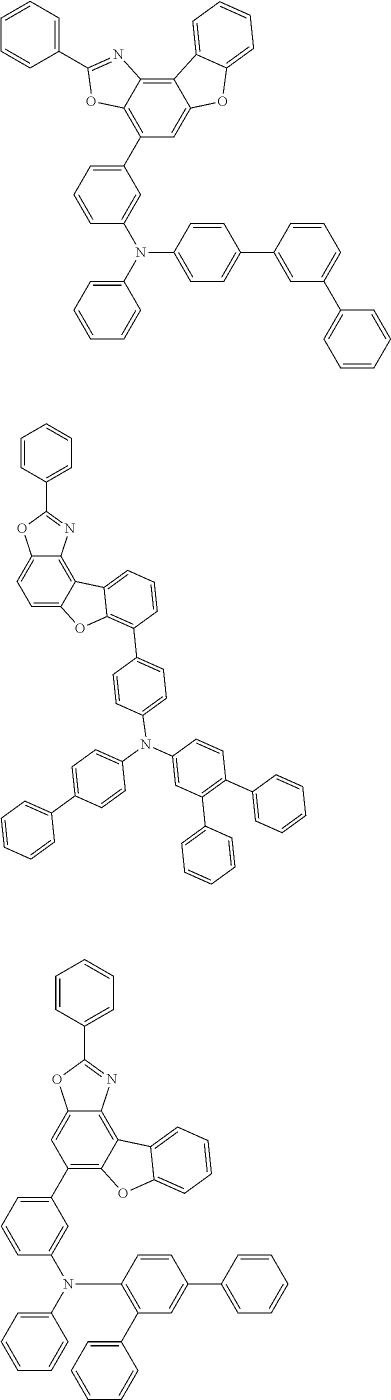 [SMILES: c1ccc(-c2cccc(-c3ccc(N(c4ccccc4)c4cccc(-c5cc6oc7ccccc7c6c6nc(-c7ccccc7)oc56)c4)cc3)c2)cc1 ; c1ccc(-c2ccc(N(c3ccc(-c4cccc5c4oc4ccc6oc(-c7ccccc7)nc6c45)cc3)c3ccc(-c4ccccc4)c(-c4ccccc4)c3)cc2)cc1 ; c1ccc(-c2ccc(N(c3ccccc3)c3cccc(-c4cc5oc(-c6ccccc6)nc5c5c4oc4ccccc45)c3)c(-c3ccccc3)c2)cc1]

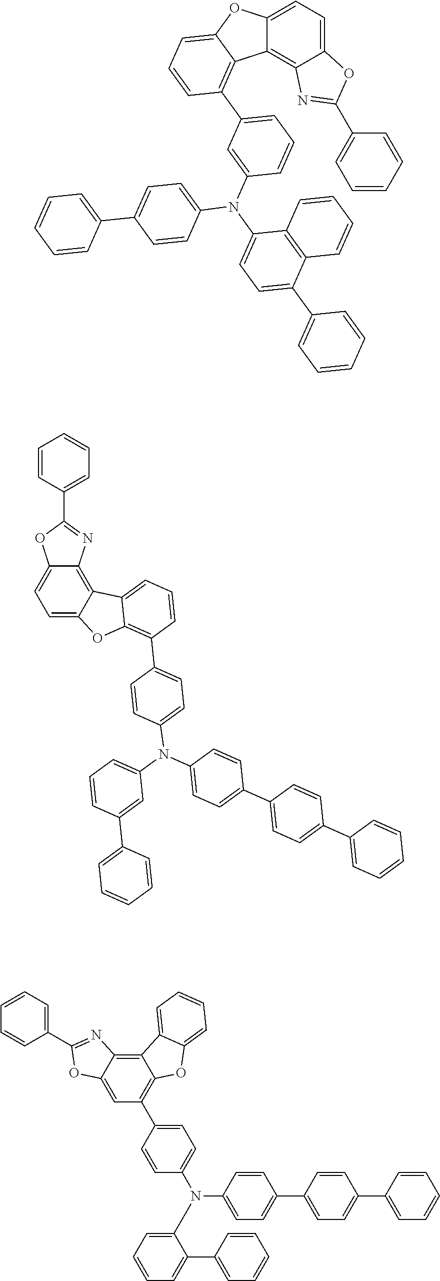 [SMILES: c1ccc(-c2ccc(N(c3cccc(-c4cccc5oc6ccc7oc(-c8ccccc8)nc7c6c45)c3)c3ccc(-c4ccccc4)c4ccccc34)cc2)cc1 ; c1ccc(-c2ccc(-c3ccc(N(c4ccc(-c5cccc6c5oc5ccc7oc(-c8ccccc8)nc7c56)cc4)c4cccc(-c5ccccc5)c4)cc3)cc2)cc1 ; c1ccc(-c2ccc(-c3ccc(N(c4ccc(-c5cc6oc(-c7ccccc7)nc6c6c5oc5ccccc56)cc4)c4ccccc4-c4ccccc4)cc3)cc2)cc1]

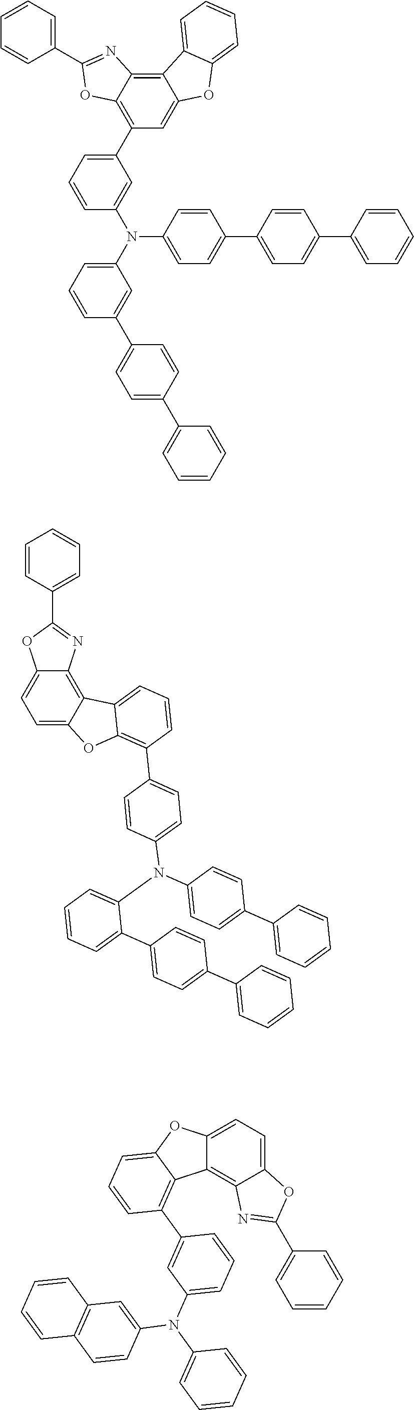 [SMILES: c1ccc(-c2ccc(-c3ccc(N(c4cccc(-c5ccc(-c6ccccc6)cc5)c4)c4cccc(-c5cc6oc7ccccc7c6c6nc(-c7ccccc7)oc56)c4)cc3)cc2)cc1 ; c1ccc(-c2ccc(-c3ccccc3N(c3ccc(-c4ccccc4)cc3)c3ccc(-c4cccc5c4oc4ccc6oc(-c7ccccc7)nc6c45)cc3)cc2)cc1 ; c1ccc(-c2nc3c(ccc4oc5cccc(-c6cccc(N(c7ccccc7)c7ccc8ccccc8c7)c6)c5c43)o2)cc1]

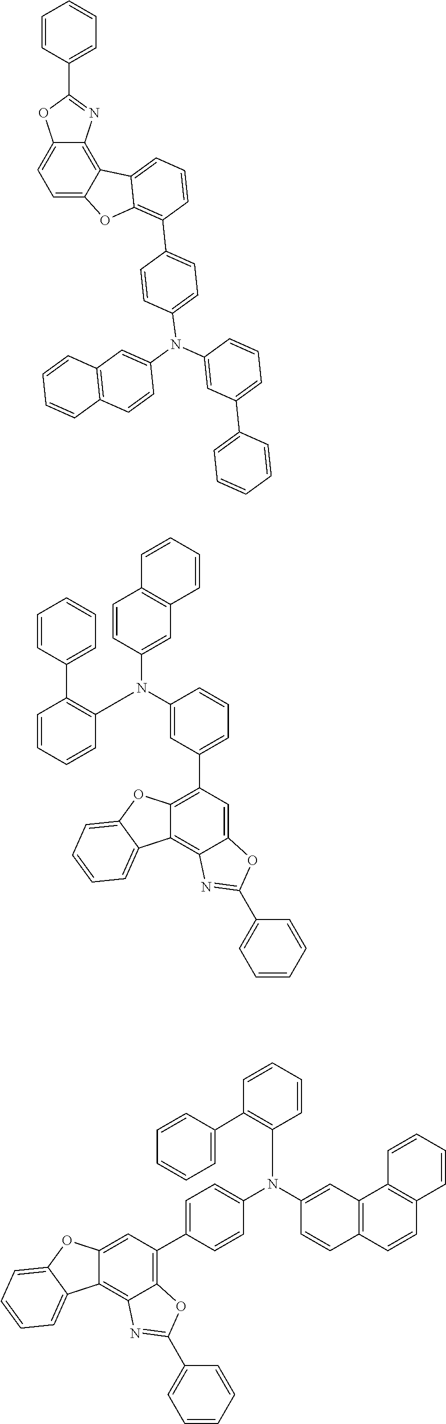 [SMILES: c1ccc(-c2cccc(N(c3ccc(-c4cccc5c4oc4ccc6oc(-c7ccccc7)nc6c45)cc3)c3ccc4ccccc4c3)c2)cc1 ; c1ccc(-c2nc3c(cc(-c4cccc(N(c5ccc6ccccc6c5)c5ccccc5-c5ccccc5)c4)c4oc5ccccc5c43)o2)cc1 ; c1ccc(-c2nc3c(o2)c(-c2ccc(N(c4ccc5ccc6ccccc6c5c4)c4ccccc4-c4ccccc4)cc2)cc2oc4ccccc4c23)cc1]

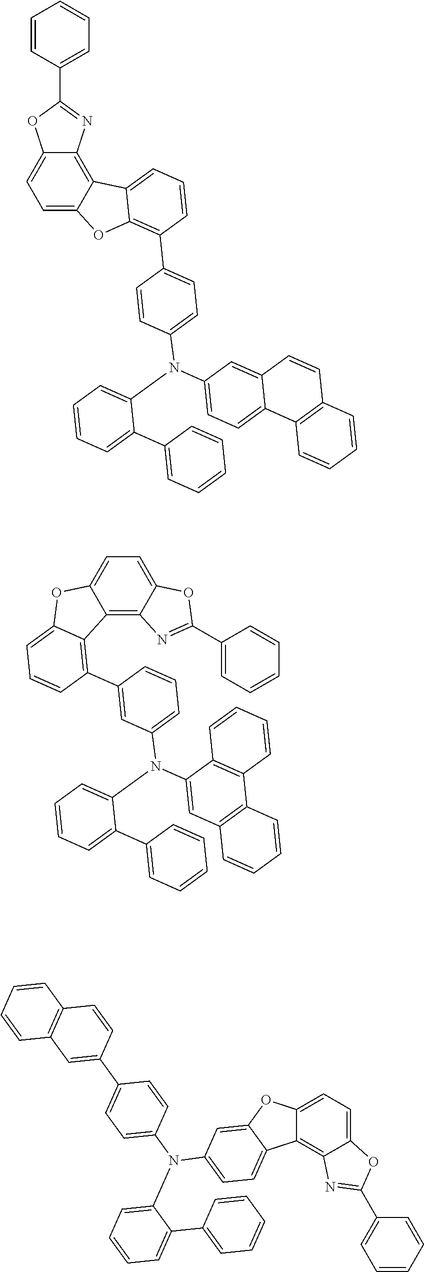 [SMILES: c1ccc(-c2nc3c(ccc4oc5c(-c6ccc(N(c7ccc8c(ccc9ccccc98)c7)c7ccccc7-c7ccccc7)cc6)cccc5c43)o2)cc1 ; c1ccc(-c2nc3c(ccc4oc5cccc(-c6cccc(N(c7ccccc7-c7ccccc7)c7cc8ccccc8c8ccccc78)c6)c5c43)o2)cc1 ; c1ccc(-c2nc3c(ccc4oc5cc(N(c6ccc(-c7ccc8ccccc8c7)cc6)c6ccccc6-c6ccccc6)ccc5c43)o2)cc1]

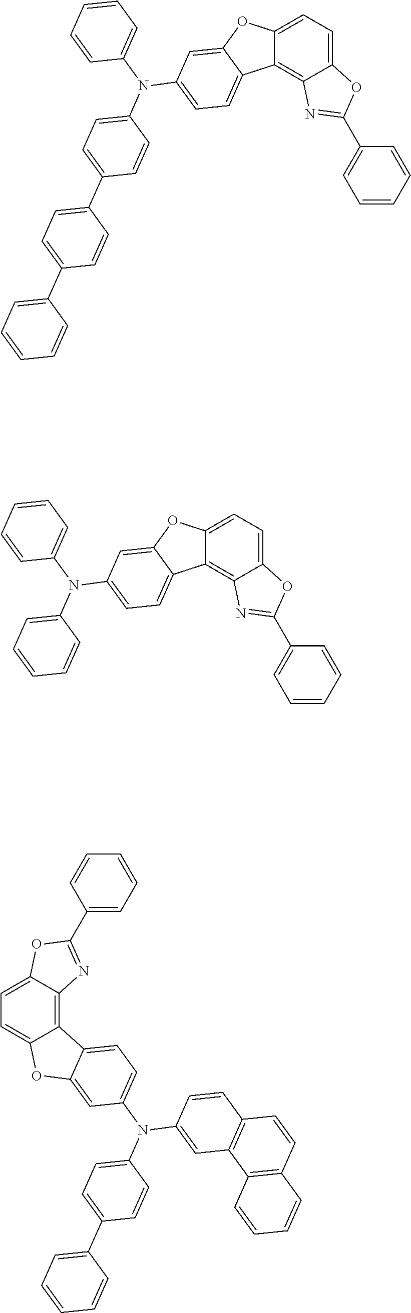 [SMILES: c1ccc(-c2ccc(-c3ccc(N(c4ccccc4)c4ccc5c(c4)oc4ccc6oc(-c7ccccc7)nc6c45)cc3)cc2)cc1 ; c1ccc(-c2nc3c(ccc4oc5cc(N(c6ccccc6)c6ccccc6)ccc5c43)o2)cc1 ; c1ccc(-c2ccc(N(c3ccc4c(c3)oc3ccc5oc(-c6ccccc6)nc5c34)c3ccc4ccc5ccccc5c4c3)cc2)cc1]

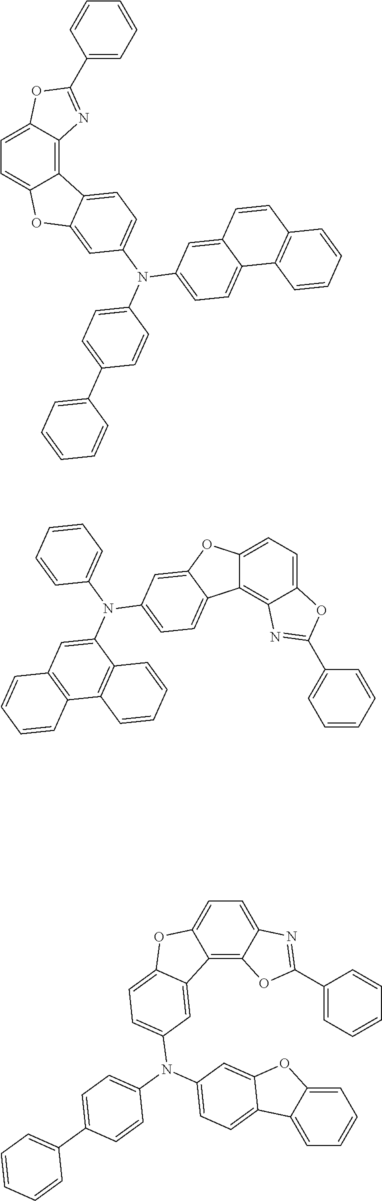 [SMILES: c1ccc(-c2ccc(N(c3ccc4c(ccc5ccccc54)c3)c3ccc4c(c3)oc3ccc5oc(-c6ccccc6)nc5c34)cc2)cc1 ; c1ccc(-c2nc3c(ccc4oc5cc(N(c6ccccc6)c6cc7ccccc7c7ccccc67)ccc5c43)o2)cc1 ; c1ccc(-c2ccc(N(c3ccc4c(c3)oc3ccccc34)c3ccc4oc5ccc6nc(-c7ccccc7)oc6c5c4c3)cc2)cc1]

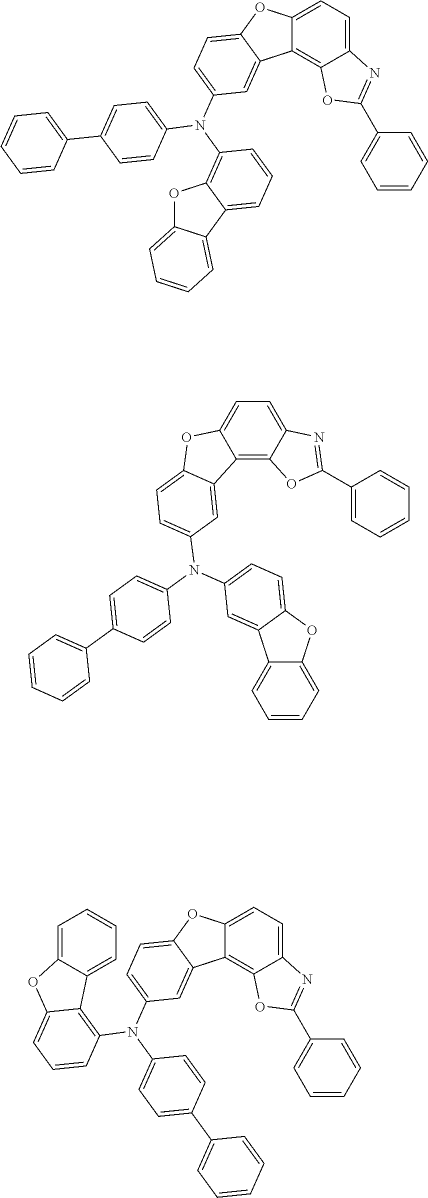 [SMILES: c1ccc(-c2ccc(N(c3ccc4oc5ccc6nc(-c7ccccc7)oc6c5c4c3)c3cccc4c3oc3ccccc34)cc2)cc1 ; c1ccc(-c2ccc(N(c3ccc4oc5ccccc5c4c3)c3ccc4oc5ccc6nc(-c7ccccc7)oc6c5c4c3)cc2)cc1 ; c1ccc(-c2ccc(N(c3ccc4oc5ccc6nc(-c7ccccc7)oc6c5c4c3)c3cccc4oc5ccccc5c34)cc2)cc1]

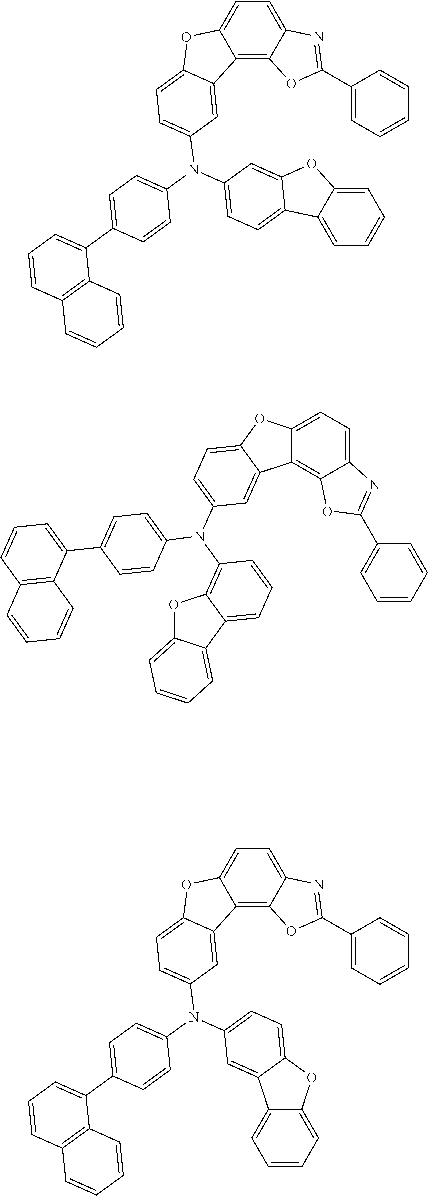 [SMILES: c1ccc(-c2nc3ccc4oc5ccc(N(c6ccc(-c7cccc8ccccc78)cc6)c6ccc7c(c6)oc6ccccc67)cc5c4c3o2)cc1 ; c1ccc(-c2nc3ccc4oc5ccc(N(c6ccc(-c7cccc8ccccc78)cc6)c6cccc7c6oc6ccccc67)cc5c4c3o2)cc1 ; c1ccc(-c2nc3ccc4oc5ccc(N(c6ccc(-c7cccc8ccccc78)cc6)c6ccc7oc8ccccc8c7c6)cc5c4c3o2)cc1]

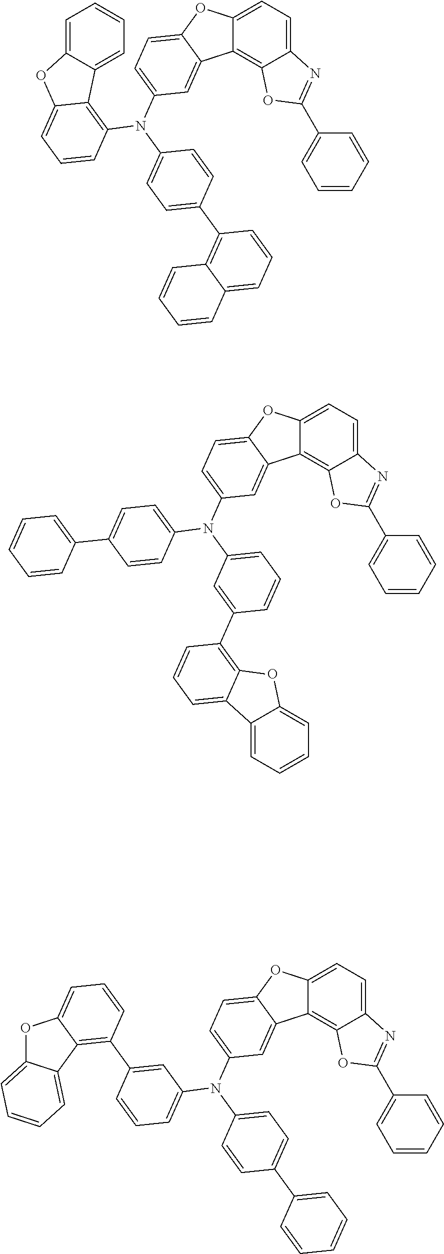 [SMILES: c1ccc(-c2nc3ccc4oc5ccc(N(c6ccc(-c7cccc8ccccc78)cc6)c6cccc7oc8ccccc8c67)cc5c4c3o2)cc1 ; c1ccc(-c2ccc(N(c3cccc(-c4cccc5c4oc4ccccc45)c3)c3ccc4oc5ccc6nc(-c7ccccc7)oc6c5c4c3)cc2)cc1 ; c1ccc(-c2ccc(N(c3cccc(-c4cccc5oc6ccccc6c45)c3)c3ccc4oc5ccc6nc(-c7ccccc7)oc6c5c4c3)cc2)cc1]

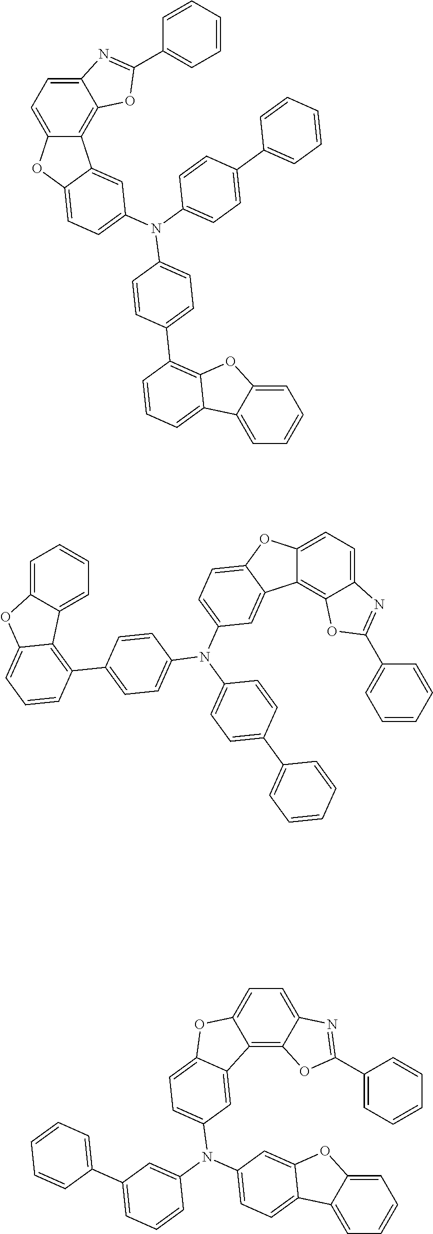 [SMILES: c1ccc(-c2ccc(N(c3ccc(-c4cccc5c4oc4ccccc45)cc3)c3ccc4oc5ccc6nc(-c7ccccc7)oc6c5c4c3)cc2)cc1 ; c1ccc(-c2ccc(N(c3ccc(-c4cccc5oc6ccccc6c45)cc3)c3ccc4oc5ccc6nc(-c7ccccc7)oc6c5c4c3)cc2)cc1 ; c1ccc(-c2cccc(N(c3ccc4c(c3)oc3ccccc34)c3ccc4oc5ccc6nc(-c7ccccc7)oc6c5c4c3)c2)cc1]

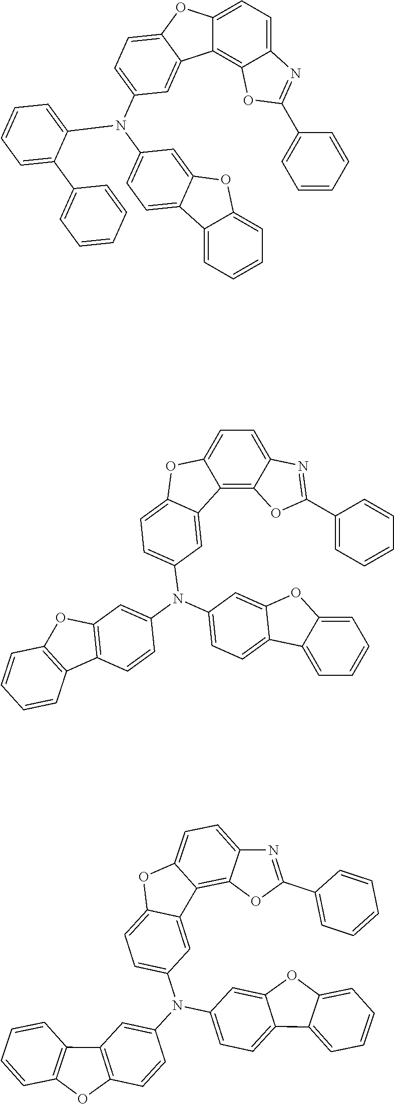 [SMILES: c1ccc(-c2nc3ccc4oc5ccc(N(c6ccc7c(c6)oc6ccccc67)c6ccccc6-c6ccccc6)cc5c4c3o2)cc1 ; c1ccc(-c2nc3ccc4oc5ccc(N(c6ccc7c(c6)oc6ccccc67)c6ccc7c(c6)oc6ccccc67)cc5c4c3o2)cc1 ; c1ccc(-c2nc3ccc4oc5ccc(N(c6ccc7c(c6)oc6ccccc67)c6ccc7oc8ccccc8c7c6)cc5c4c3o2)cc1]

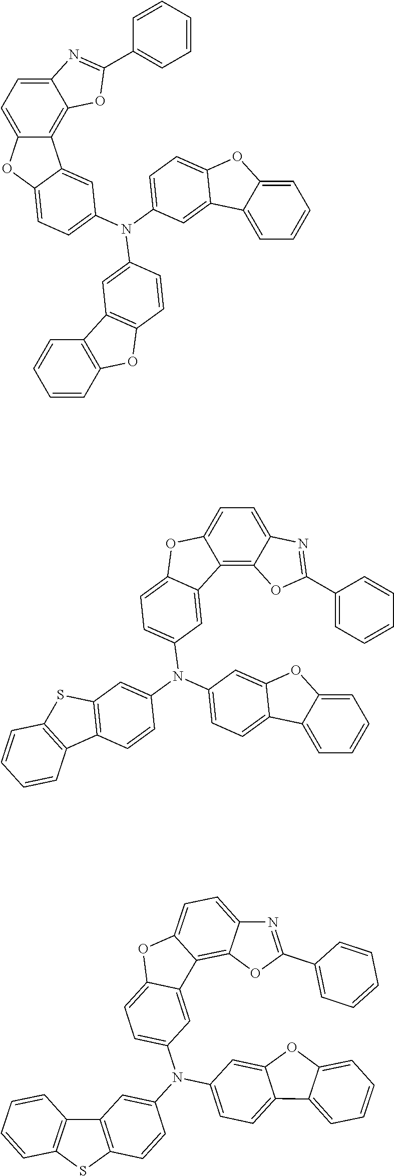 [SMILES: c1ccc(-c2nc3ccc4oc5ccc(N(c6ccc7oc8ccccc8c7c6)c6ccc7oc8ccccc8c7c6)cc5c4c3o2)cc1 ; c1ccc(-c2nc3ccc4oc5ccc(N(c6ccc7c(c6)oc6ccccc67)c6ccc7c(c6)sc6ccccc67)cc5c4c3o2)cc1 ; c1ccc(-c2nc3ccc4oc5ccc(N(c6ccc7c(c6)oc6ccccc67)c6ccc7sc8ccccc8c7c6)cc5c4c3o2)cc1]

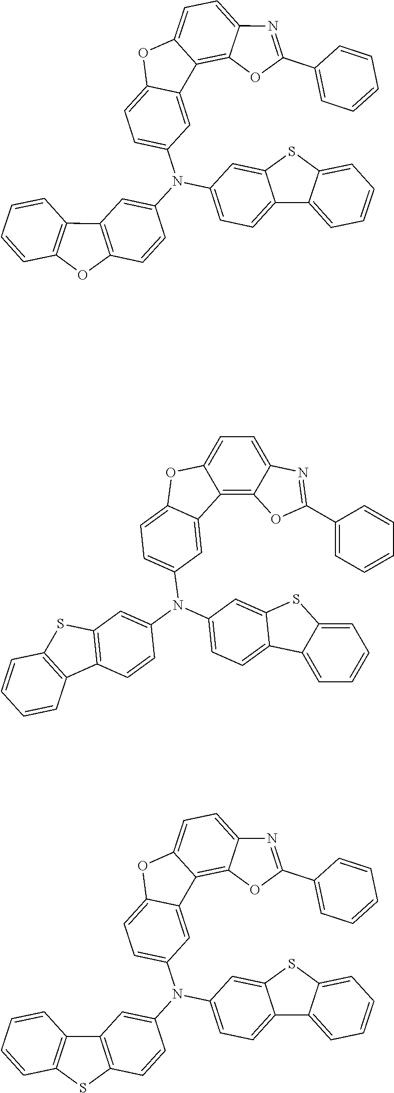 [SMILES: c1ccc(-c2nc3ccc4oc5ccc(N(c6ccc7c(c6)sc6ccccc67)c6ccc7oc8ccccc8c7c6)cc5c4c3o2)cc1 ; c1ccc(-c2nc3ccc4oc5ccc(N(c6ccc7c(c6)sc6ccccc67)c6ccc7c(c6)sc6ccccc67)cc5c4c3o2)cc1 ; c1ccc(-c2nc3ccc4oc5ccc(N(c6ccc7c(c6)sc6ccccc67)c6ccc7sc8ccccc8c7c6)cc5c4c3o2)cc1]

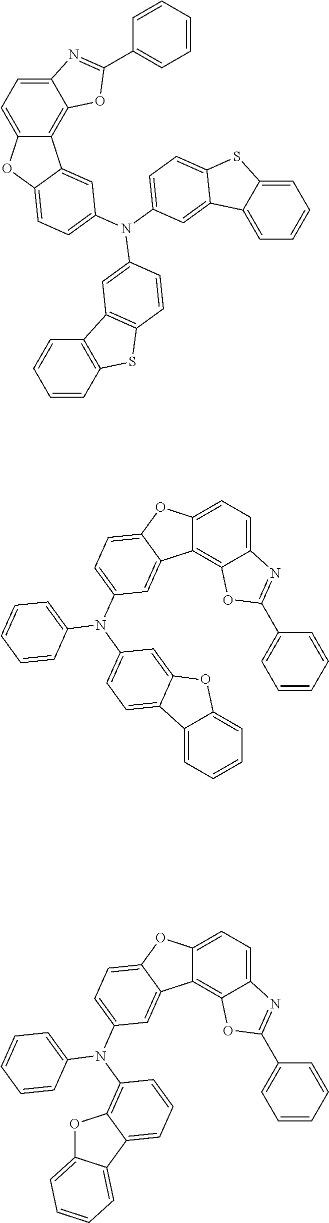 [SMILES: c1ccc(-c2nc3ccc4oc5ccc(N(c6ccc7sc8ccccc8c7c6)c6ccc7sc8ccccc8c7c6)cc5c4c3o2)cc1 ; c1ccc(-c2nc3ccc4oc5ccc(N(c6ccccc6)c6ccc7c(c6)oc6ccccc67)cc5c4c3o2)cc1 ; c1ccc(-c2nc3ccc4oc5ccc(N(c6ccccc6)c6cccc7c6oc6ccccc67)cc5c4c3o2)cc1]

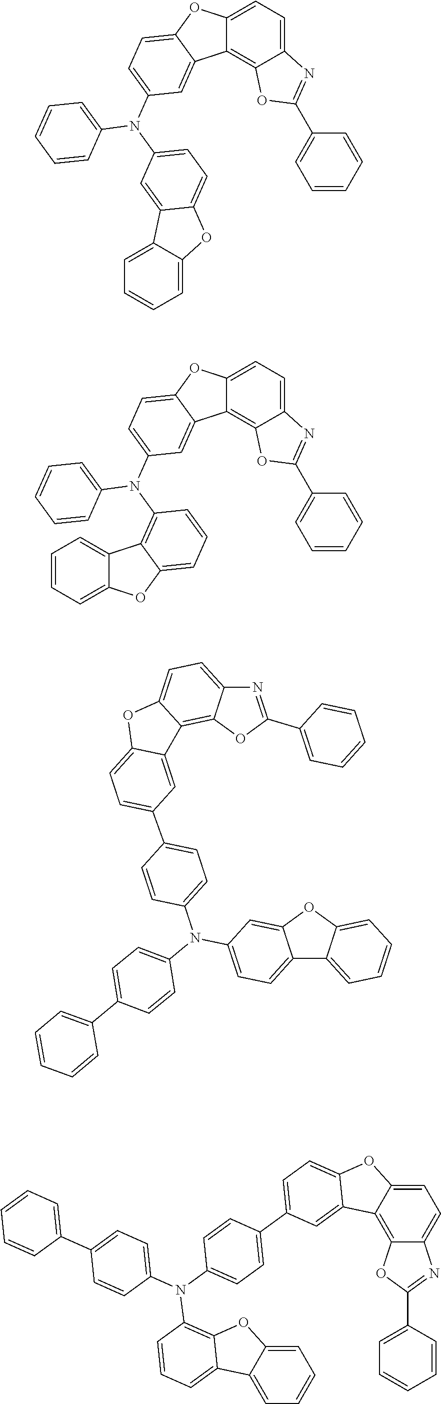 [SMILES: c1ccc(-c2nc3ccc4oc5ccc(N(c6ccccc6)c6ccc7oc8ccccc8c7c6)cc5c4c3o2)cc1 ; c1ccc(-c2nc3ccc4oc5ccc(N(c6ccccc6)c6cccc7oc8ccccc8c67)cc5c4c3o2)cc1 ; c1ccc(-c2ccc(N(c3ccc(-c4ccc5oc6ccc7nc(-c8ccccc8)oc7c6c5c4)cc3)c3ccc4c(c3)oc3ccccc34)cc2)cc1 ; c1ccc(-c2ccc(N(c3ccc(-c4ccc5oc6ccc7nc(-c8ccccc8)oc7c6c5c4)cc3)c3cccc4c3oc3ccccc34)cc2)cc1]

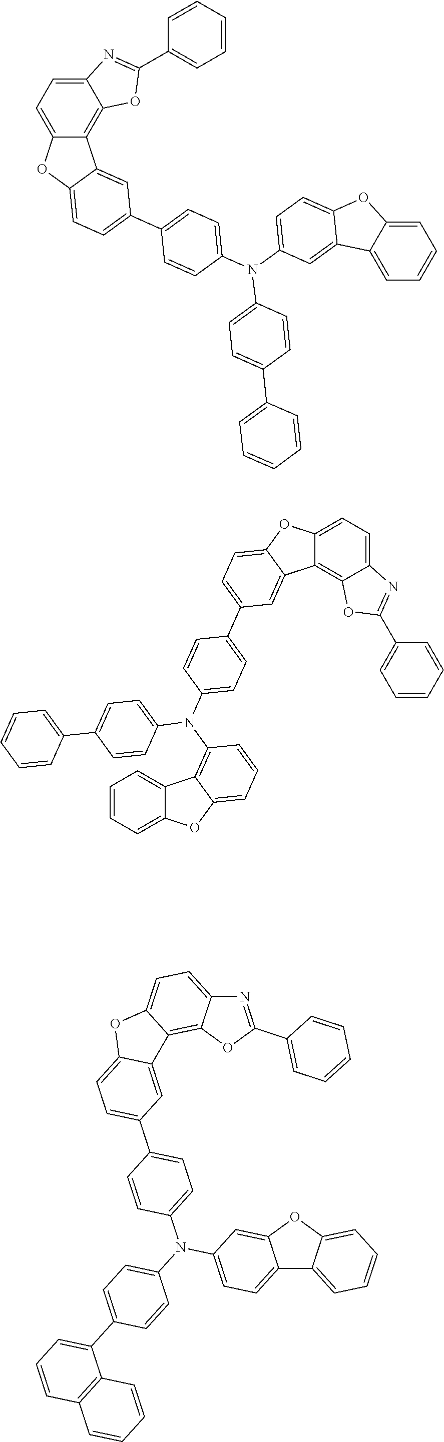 [SMILES: c1ccc(-c2ccc(N(c3ccc(-c4ccc5oc6ccc7nc(-c8ccccc8)oc7c6c5c4)cc3)c3ccc4oc5ccccc5c4c3)cc2)cc1 ; c1ccc(-c2ccc(N(c3ccc(-c4ccc5oc6ccc7nc(-c8ccccc8)oc7c6c5c4)cc3)c3cccc4oc5ccccc5c34)cc2)cc1 ; c1ccc(-c2nc3ccc4oc5ccc(-c6ccc(N(c7ccc(-c8cccc9ccccc89)cc7)c7ccc8c(c7)oc7ccccc78)cc6)cc5c4c3o2)cc1]

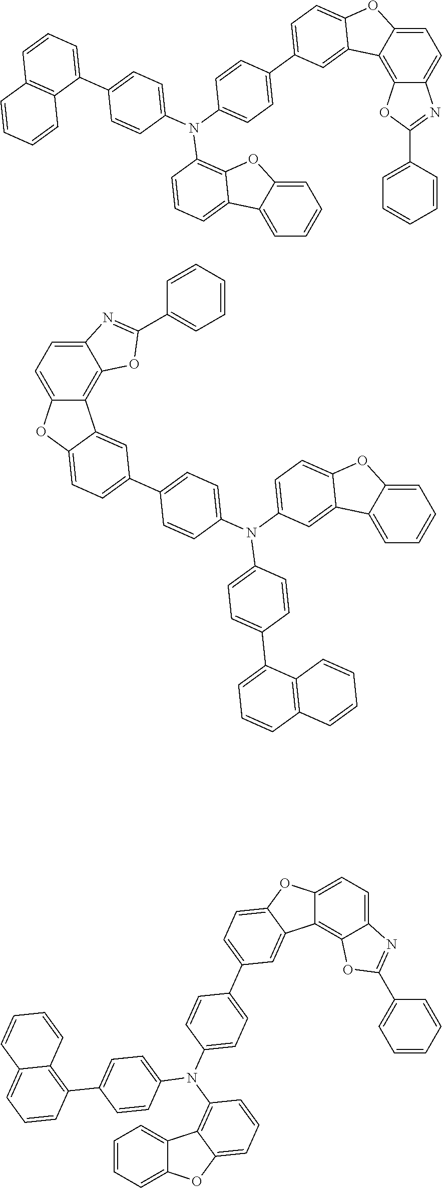 [SMILES: c1ccc(-c2nc3ccc4oc5ccc(-c6ccc(N(c7ccc(-c8cccc9ccccc89)cc7)c7cccc8c7oc7ccccc78)cc6)cc5c4c3o2)cc1 ; c1ccc(-c2nc3ccc4oc5ccc(-c6ccc(N(c7ccc(-c8cccc9ccccc89)cc7)c7ccc8oc9ccccc9c8c7)cc6)cc5c4c3o2)cc1 ; c1ccc(-c2nc3ccc4oc5ccc(-c6ccc(N(c7ccc(-c8cccc9ccccc89)cc7)c7cccc8oc9ccccc9c78)cc6)cc5c4c3o2)cc1]

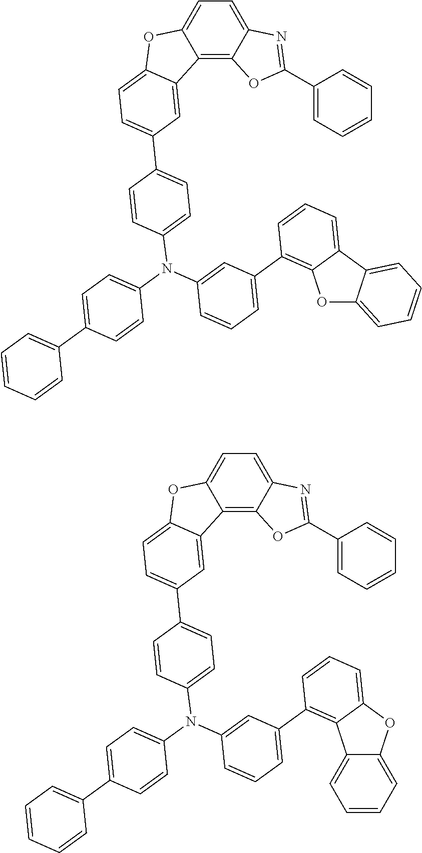 [SMILES: c1ccc(-c2ccc(N(c3ccc(-c4ccc5oc6ccc7nc(-c8ccccc8)oc7c6c5c4)cc3)c3cccc(-c4cccc5c4oc4ccccc45)c3)cc2)cc1 ; c1ccc(-c2ccc(N(c3ccc(-c4ccc5oc6ccc7nc(-c8ccccc8)oc7c6c5c4)cc3)c3cccc(-c4cccc5oc6ccccc6c45)c3)cc2)cc1]

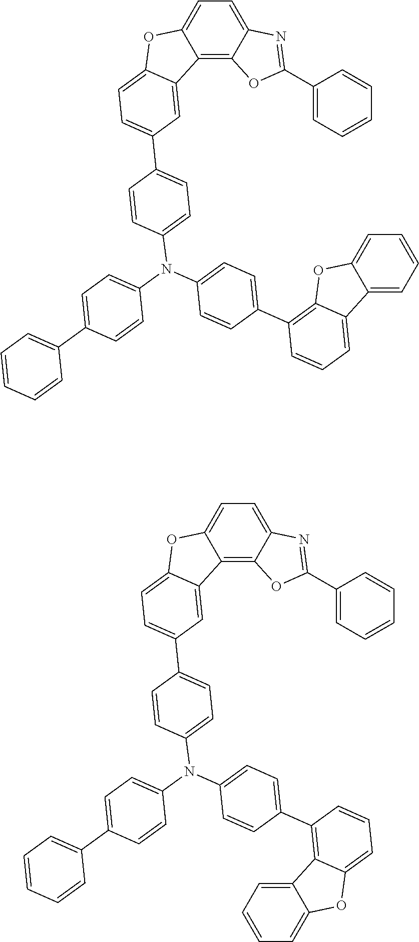 [SMILES: c1ccc(-c2ccc(N(c3ccc(-c4ccc5oc6ccc7nc(-c8ccccc8)oc7c6c5c4)cc3)c3ccc(-c4cccc5c4oc4ccccc45)cc3)cc2)cc1 ; c1ccc(-c2ccc(N(c3ccc(-c4ccc5oc6ccc7nc(-c8ccccc8)oc7c6c5c4)cc3)c3ccc(-c4cccc5oc6ccccc6c45)cc3)cc2)cc1]

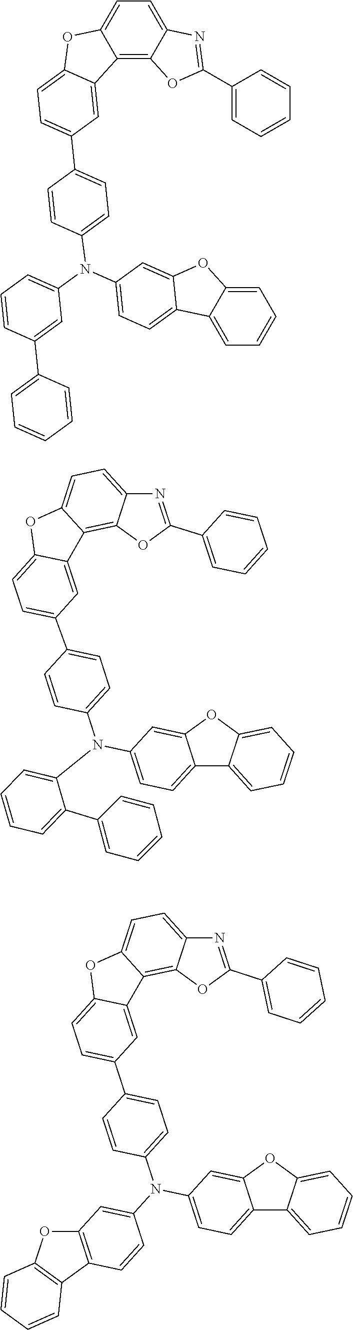

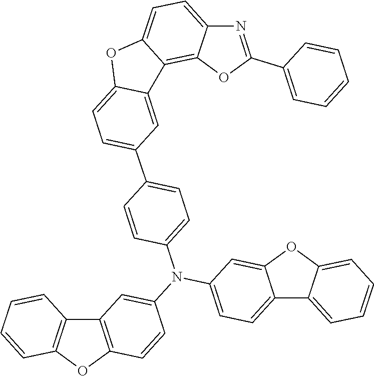 [SMILES: c1ccc(-c2nc3ccc4oc5ccc(-c6ccc(N(c7ccc8c(c7)oc7ccccc78)c7ccc8oc9ccccc9c8c7)cc6)cc5c4c3o2)cc1]

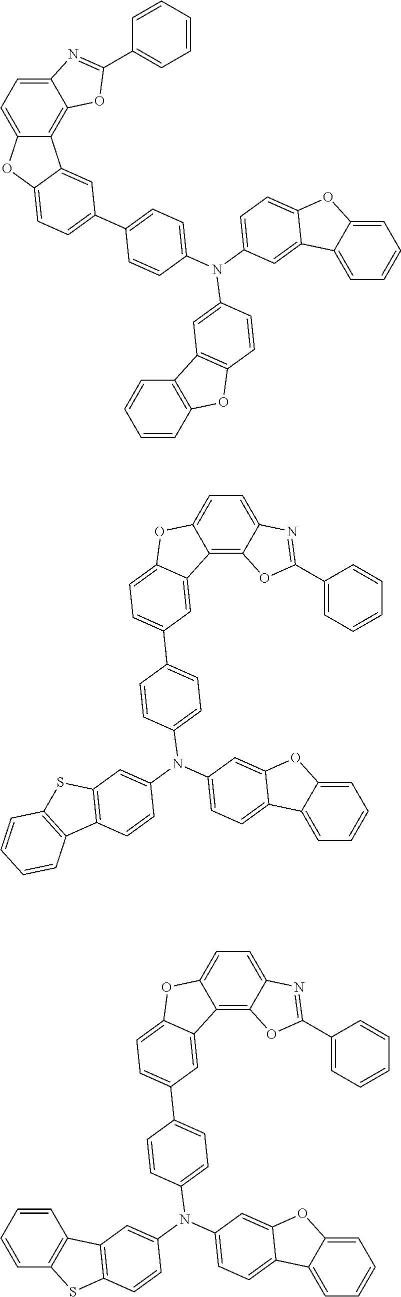 [SMILES: c1ccc(-c2nc3ccc4oc5ccc(-c6ccc(N(c7ccc8oc9ccccc9c8c7)c7ccc8oc9ccccc9c8c7)cc6)cc5c4c3o2)cc1 ; c1ccc(-c2nc3ccc4oc5ccc(-c6ccc(N(c7ccc8c(c7)oc7ccccc78)c7ccc8c(c7)sc7ccccc78)cc6)cc5c4c3o2)cc1 ; c1ccc(-c2nc3ccc4oc5ccc(-c6ccc(N(c7ccc8c(c7)oc7ccccc78)c7ccc8sc9ccccc9c8c7)cc6)cc5c4c3o2)cc1]

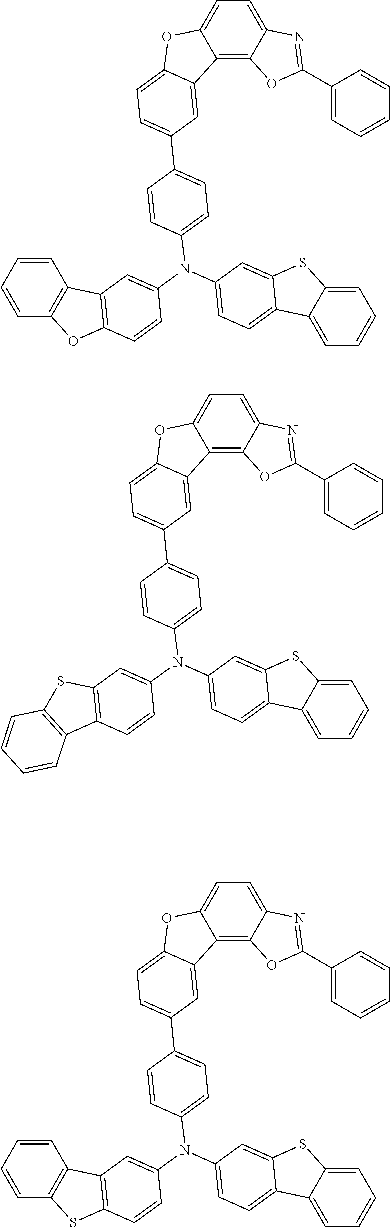 [SMILES: c1ccc(-c2nc3ccc4oc5ccc(-c6ccc(N(c7ccc8c(c7)sc7ccccc78)c7ccc8oc9ccccc9c8c7)cc6)cc5c4c3o2)cc1 ; c1ccc(-c2nc3ccc4oc5ccc(-c6ccc(N(c7ccc8c(c7)sc7ccccc78)c7ccc8c(c7)sc7ccccc78)cc6)cc5c4c3o2)cc1 ; c1ccc(-c2nc3ccc4oc5ccc(-c6ccc(N(c7ccc8c(c7)sc7ccccc78)c7ccc8sc9ccccc9c8c7)cc6)cc5c4c3o2)cc1]

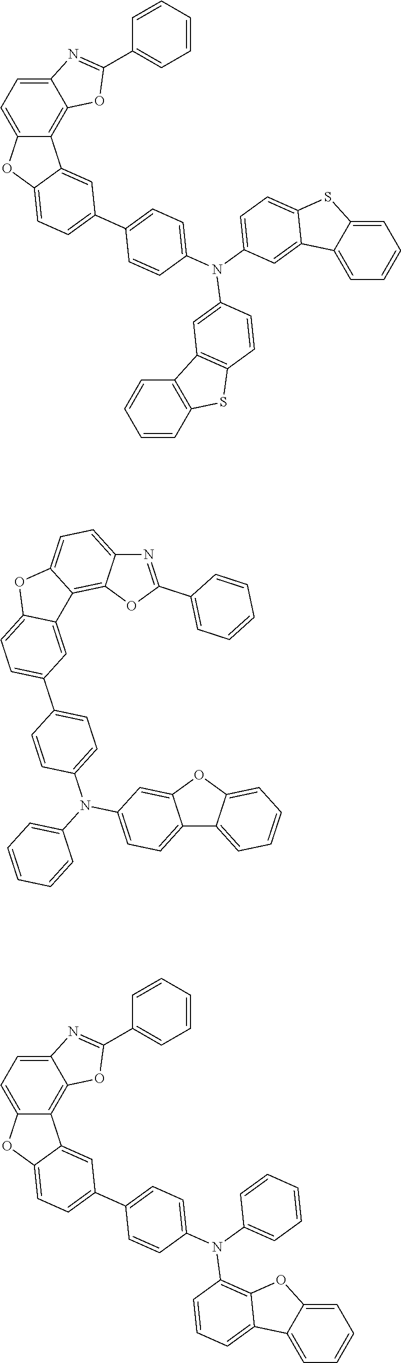 [SMILES: c1ccc(-c2nc3ccc4oc5ccc(-c6ccc(N(c7ccc8sc9ccccc9c8c7)c7ccc8sc9ccccc9c8c7)cc6)cc5c4c3o2)cc1 ; c1ccc(-c2nc3ccc4oc5ccc(-c6ccc(N(c7ccccc7)c7ccc8c(c7)oc7ccccc78)cc6)cc5c4c3o2)cc1 ; c1ccc(-c2nc3ccc4oc5ccc(-c6ccc(N(c7ccccc7)c7cccc8c7oc7ccccc78)cc6)cc5c4c3o2)cc1]

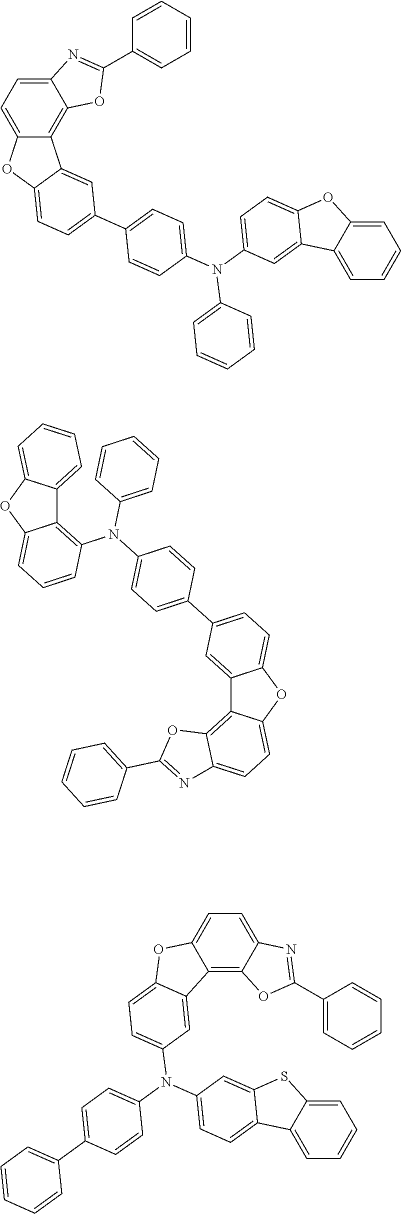 [SMILES: c1ccc(-c2nc3ccc4oc5ccc(-c6ccc(N(c7ccccc7)c7ccc8oc9ccccc9c8c7)cc6)cc5c4c3o2)cc1 ; c1ccc(-c2nc3ccc4oc5ccc(-c6ccc(N(c7ccccc7)c7cccc8oc9ccccc9c78)cc6)cc5c4c3o2)cc1 ; c1ccc(-c2ccc(N(c3ccc4c(c3)sc3ccccc34)c3ccc4oc5ccc6nc(-c7ccccc7)oc6c5c4c3)cc2)cc1]

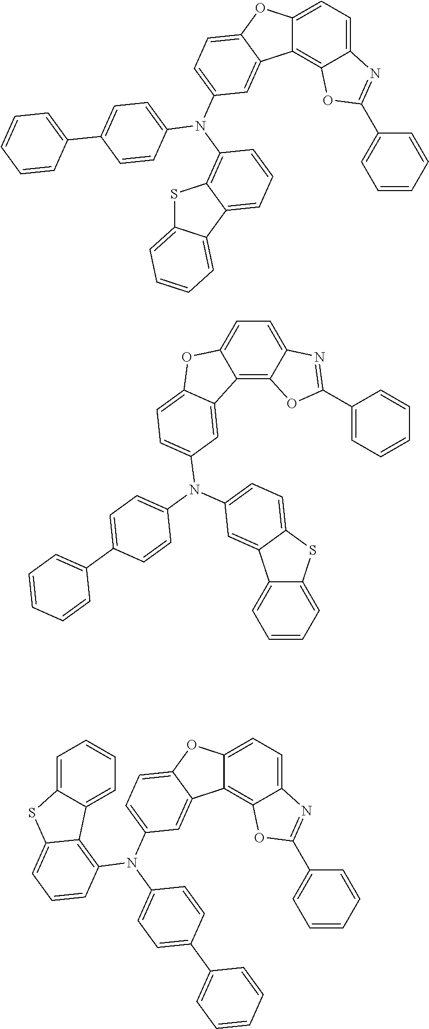 [SMILES: c1ccc(-c2ccc(N(c3ccc4oc5ccc6nc(-c7ccccc7)oc6c5c4c3)c3cccc4c3sc3ccccc34)cc2)cc1 ; c1ccc(-c2ccc(N(c3ccc4sc5ccccc5c4c3)c3ccc4oc5ccc6nc(-c7ccccc7)oc6c5c4c3)cc2)cc1 ; c1ccc(-c2ccc(N(c3ccc4oc5ccc6nc(-c7ccccc7)oc6c5c4c3)c3cccc4sc5ccccc5c34)cc2)cc1]

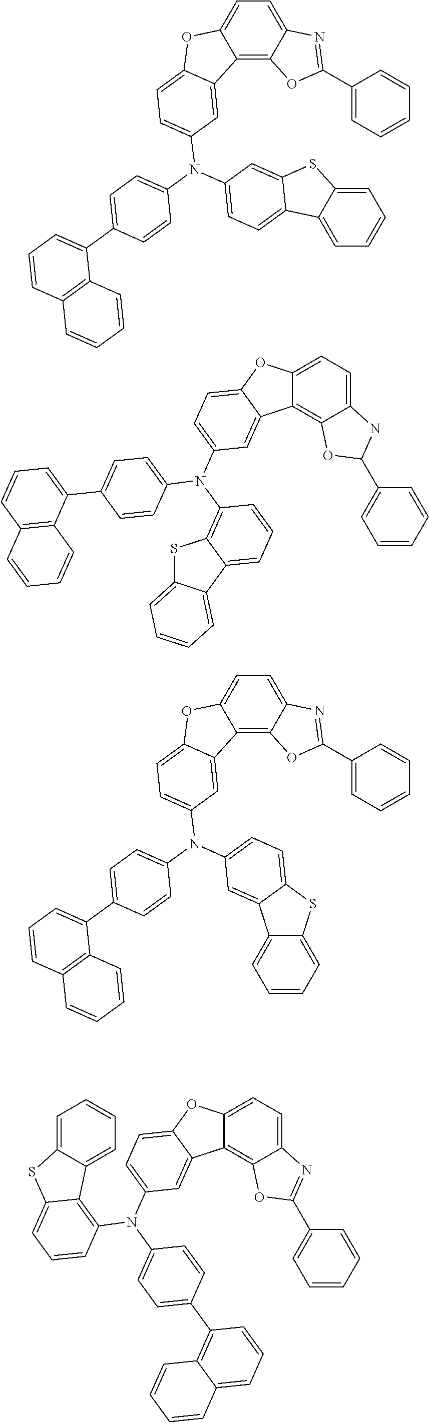

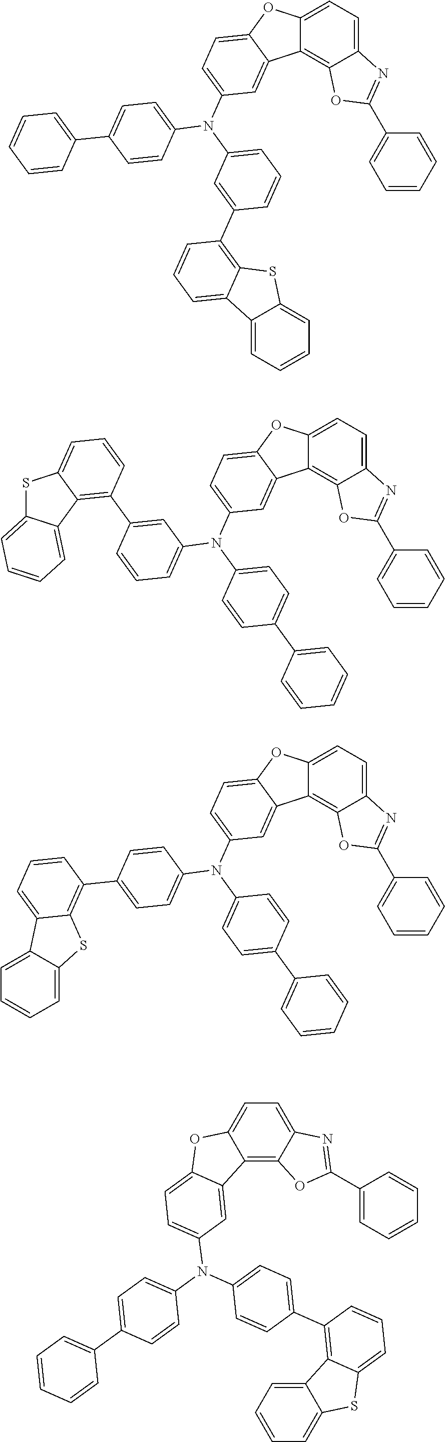

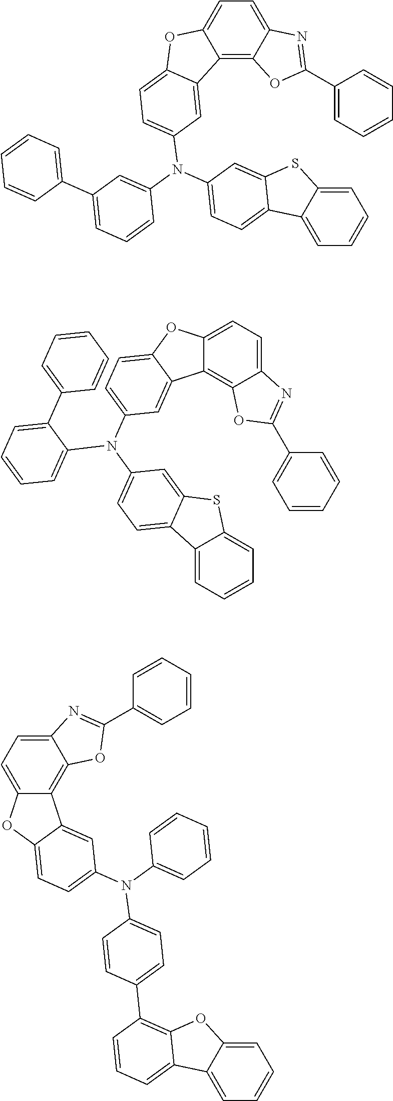 [SMILES: c1ccc(-c2cccc(N(c3ccc4c(c3)sc3ccccc34)c3ccc4oc5ccc6nc(-c7ccccc7)oc6c5c4c3)c2)cc1 ; c1ccc(-c2nc3ccc4oc5ccc(N(c6ccc7c(c6)sc6ccccc67)c6ccccc6-c6ccccc6)cc5c4c3o2)cc1 ; c1ccc(-c2nc3ccc4oc5ccc(N(c6ccccc6)c6ccc(-c7cccc8c7oc7ccccc78)cc6)cc5c4c3o2)cc1]

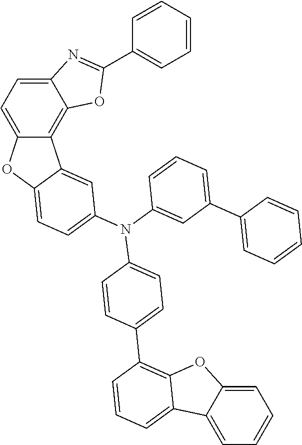 [SMILES: c1ccc(-c2cccc(N(c3ccc(-c4cccc5c4oc4ccccc45)cc3)c3ccc4oc5ccc6nc(-c7ccccc7)oc6c5c4c3)c2)cc1]

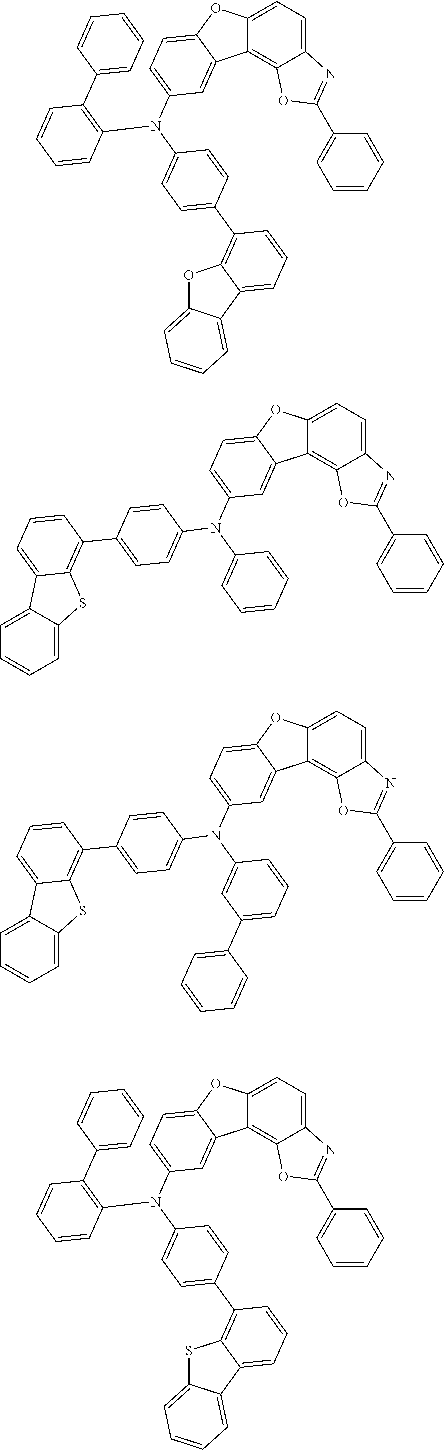 [SMILES: c1ccc(-c2nc3ccc4oc5ccc(N(c6ccc(-c7cccc8c7oc7ccccc78)cc6)c6ccccc6-c6ccccc6)cc5c4c3o2)cc1 ; c1ccc(-c2nc3ccc4oc5ccc(N(c6ccccc6)c6ccc(-c7cccc8c7sc7ccccc78)cc6)cc5c4c3o2)cc1 ; c1ccc(-c2cccc(N(c3ccc(-c4cccc5c4sc4ccccc45)cc3)c3ccc4oc5ccc6nc(-c7ccccc7)oc6c5c4c3)c2)cc1 ; c1ccc(-c2nc3ccc4oc5ccc(N(c6ccc(-c7cccc8c7sc7ccccc78)cc6)c6ccccc6-c6ccccc6)cc5c4c3o2)cc1]

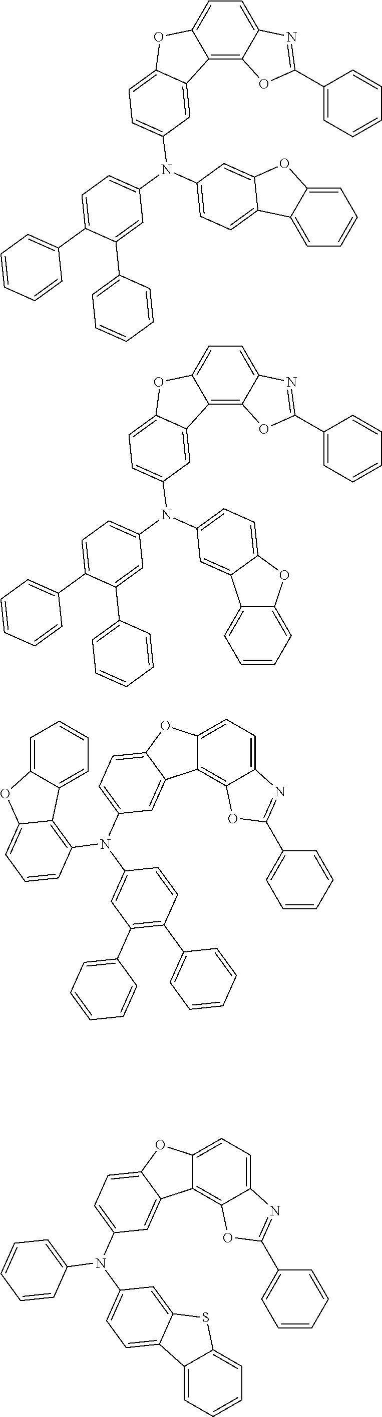 [SMILES: c1ccc(-c2nc3ccc4oc5ccc(N(c6ccc(-c7ccccc7)c(-c7ccccc7)c6)c6ccc7c(c6)oc6ccccc67)cc5c4c3o2)cc1 ; c1ccc(-c2nc3ccc4oc5ccc(N(c6ccc(-c7ccccc7)c(-c7ccccc7)c6)c6ccc7oc8ccccc8c7c6)cc5c4c3o2)cc1 ; c1ccc(-c2nc3ccc4oc5ccc(N(c6ccc(-c7ccccc7)c(-c7ccccc7)c6)c6cccc7oc8ccccc8c67)cc5c4c3o2)cc1 ; c1ccc(-c2nc3ccc4oc5ccc(N(c6ccccc6)c6ccc7c(c6)sc6ccccc67)cc5c4c3o2)cc1]

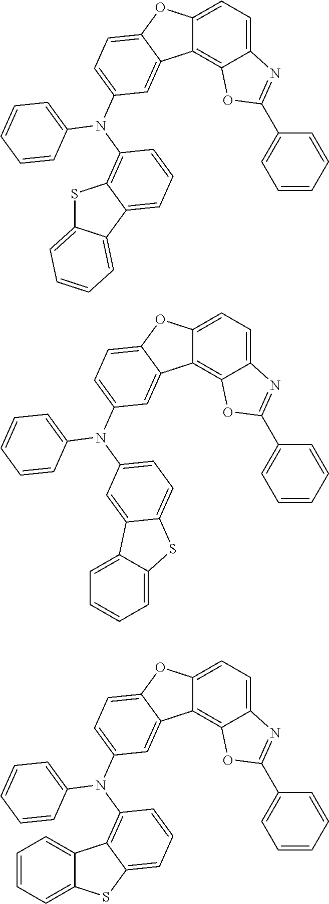 [SMILES: c1ccc(-c2nc3ccc4oc5ccc(N(c6ccccc6)c6cccc7c6sc6ccccc67)cc5c4c3o2)cc1 ; c1ccc(-c2nc3ccc4oc5ccc(N(c6ccccc6)c6ccc7sc8ccccc8c7c6)cc5c4c3o2)cc1 ; c1ccc(-c2nc3ccc4oc5ccc(N(c6ccccc6)c6cccc7sc8ccccc8c67)cc5c4c3o2)cc1]

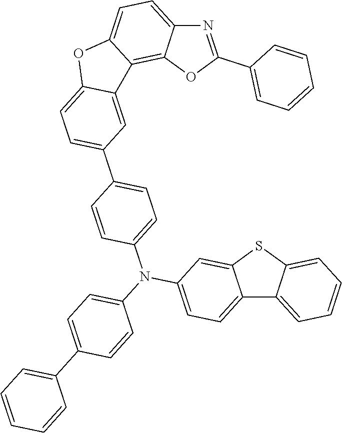 [SMILES: c1ccc(-c2ccc(N(c3ccc(-c4ccc5oc6ccc7nc(-c8ccccc8)oc7c6c5c4)cc3)c3ccc4c(c3)sc3ccccc34)cc2)cc1]

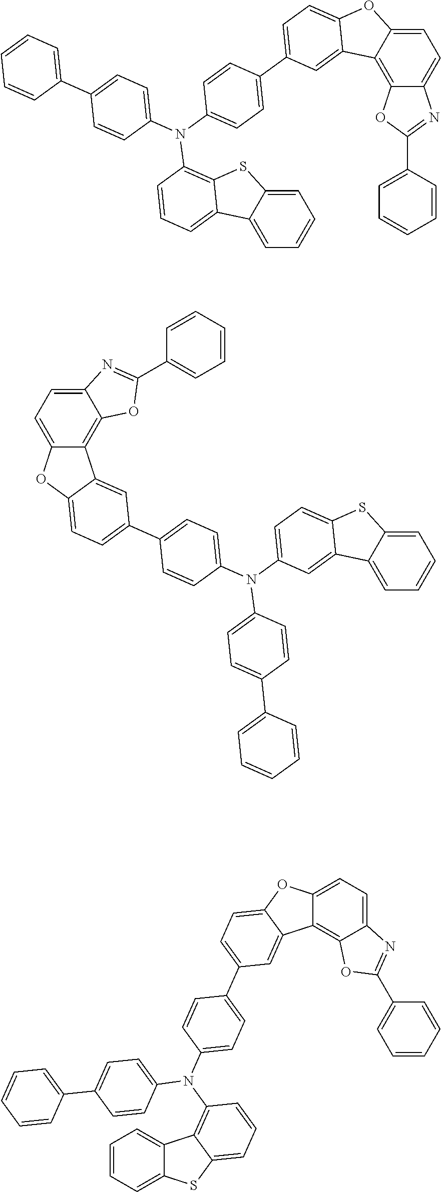 [SMILES: c1ccc(-c2ccc(N(c3ccc(-c4ccc5oc6ccc7nc(-c8ccccc8)oc7c6c5c4)cc3)c3cccc4c3sc3ccccc34)cc2)cc1 ; c1ccc(-c2ccc(N(c3ccc(-c4ccc5oc6ccc7nc(-c8ccccc8)oc7c6c5c4)cc3)c3ccc4sc5ccccc5c4c3)cc2)cc1 ; c1ccc(-c2ccc(N(c3ccc(-c4ccc5oc6ccc7nc(-c8ccccc8)oc7c6c5c4)cc3)c3cccc4sc5ccccc5c34)cc2)cc1]

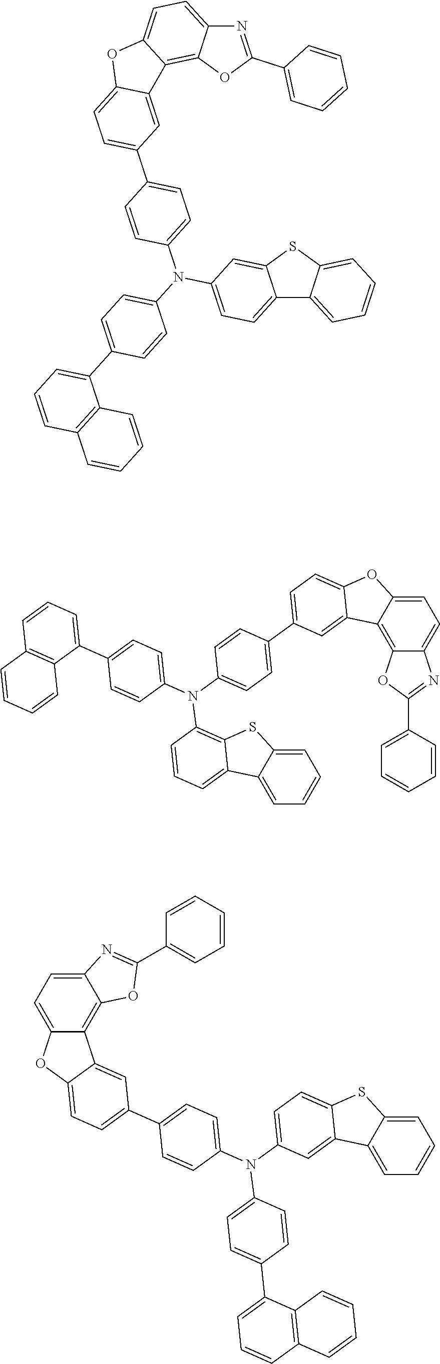 [SMILES: c1ccc(-c2nc3ccc4oc5ccc(-c6ccc(N(c7ccc(-c8cccc9ccccc89)cc7)c7ccc8c(c7)sc7ccccc78)cc6)cc5c4c3o2)cc1 ; c1ccc(-c2nc3ccc4oc5ccc(-c6ccc(N(c7ccc(-c8cccc9ccccc89)cc7)c7cccc8c7sc7ccccc78)cc6)cc5c4c3o2)cc1 ; c1ccc(-c2nc3ccc4oc5ccc(-c6ccc(N(c7ccc(-c8cccc9ccccc89)cc7)c7ccc8sc9ccccc9c8c7)cc6)cc5c4c3o2)cc1]

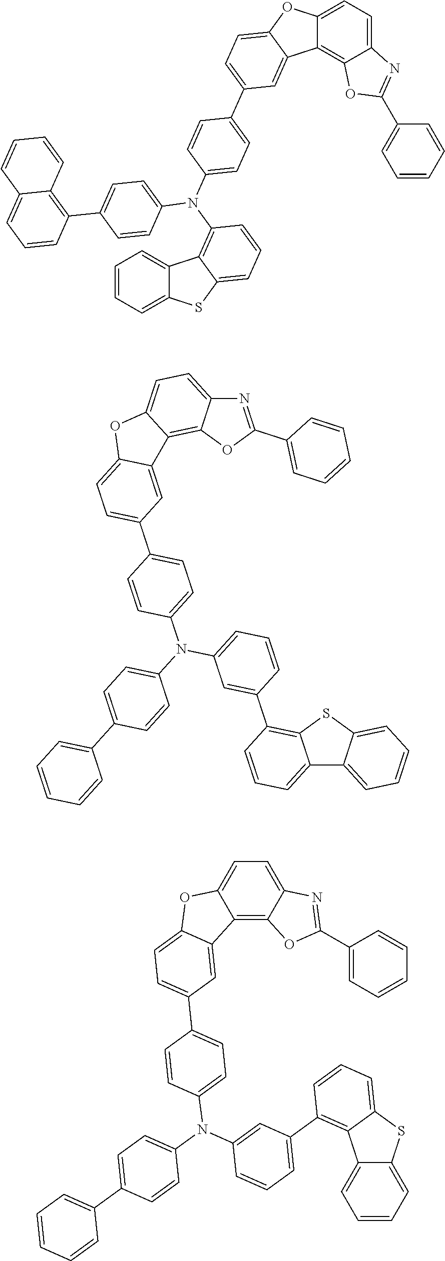 [SMILES: c1ccc(-c2nc3ccc4oc5ccc(-c6ccc(N(c7ccc(-c8cccc9ccccc89)cc7)c7cccc8sc9ccccc9c78)cc6)cc5c4c3o2)cc1 ; c1ccc(-c2ccc(N(c3ccc(-c4ccc5oc6ccc7nc(-c8ccccc8)oc7c6c5c4)cc3)c3cccc(-c4cccc5c4sc4ccccc45)c3)cc2)cc1 ; c1ccc(-c2ccc(N(c3ccc(-c4ccc5oc6ccc7nc(-c8ccccc8)oc7c6c5c4)cc3)c3cccc(-c4cccc5sc6ccccc6c45)c3)cc2)cc1]

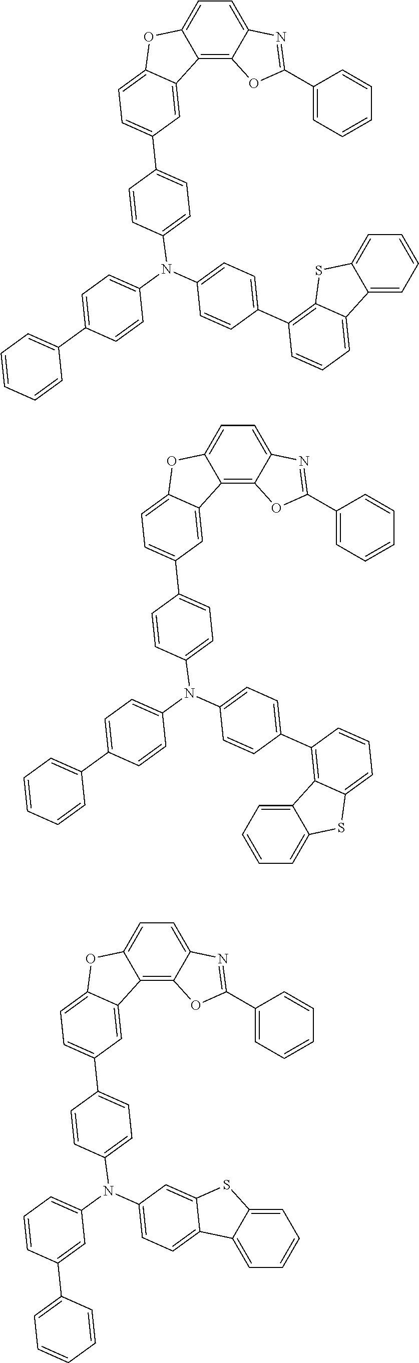 [SMILES: c1ccc(-c2ccc(N(c3ccc(-c4ccc5oc6ccc7nc(-c8ccccc8)oc7c6c5c4)cc3)c3ccc(-c4cccc5c4sc4ccccc45)cc3)cc2)cc1 ; c1ccc(-c2ccc(N(c3ccc(-c4ccc5oc6ccc7nc(-c8ccccc8)oc7c6c5c4)cc3)c3ccc(-c4cccc5sc6ccccc6c45)cc3)cc2)cc1 ; c1ccc(-c2cccc(N(c3ccc(-c4ccc5oc6ccc7nc(-c8ccccc8)oc7c6c5c4)cc3)c3ccc4c(c3)sc3ccccc34)c2)cc1]

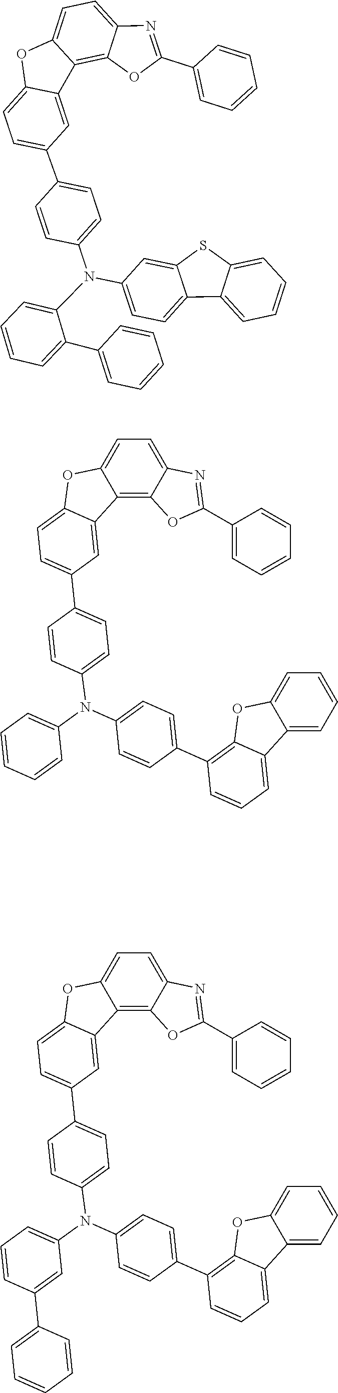 [SMILES: c1ccc(-c2nc3ccc4oc5ccc(-c6ccc(N(c7ccc8c(c7)sc7ccccc78)c7ccccc7-c7ccccc7)cc6)cc5c4c3o2)cc1 ; c1ccc(-c2nc3ccc4oc5ccc(-c6ccc(N(c7ccccc7)c7ccc(-c8cccc9c8oc8ccccc89)cc7)cc6)cc5c4c3o2)cc1 ; c1ccc(-c2cccc(N(c3ccc(-c4ccc5oc6ccc7nc(-c8ccccc8)oc7c6c5c4)cc3)c3ccc(-c4cccc5c4oc4ccccc45)cc3)c2)cc1]

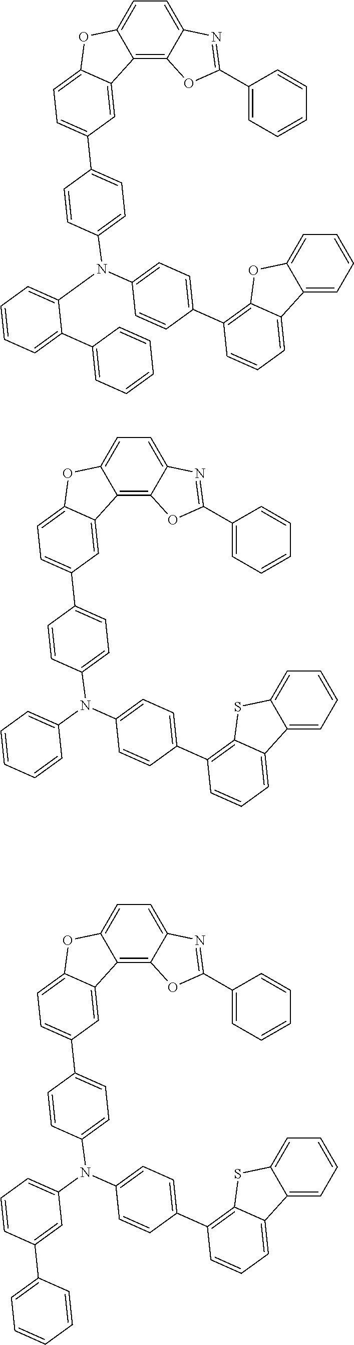

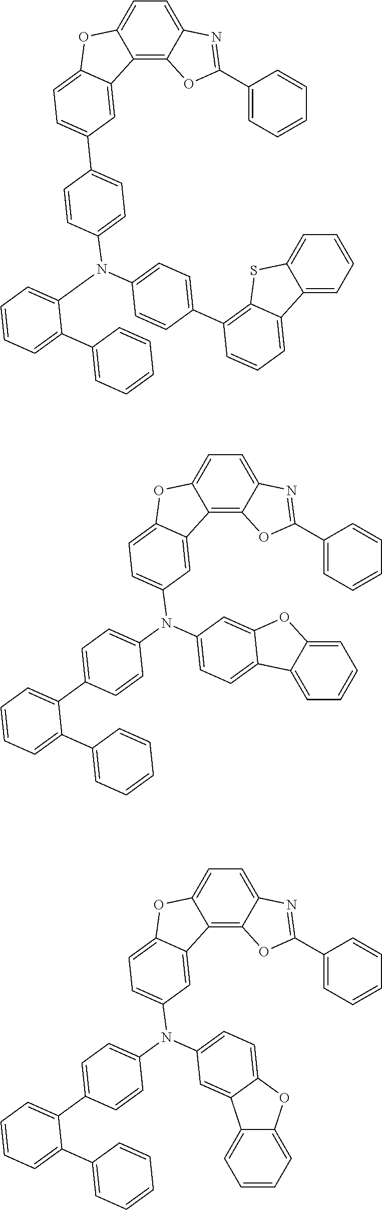 [SMILES: c1ccc(-c2nc3ccc4oc5ccc(-c6ccc(N(c7ccc(-c8cccc9c8sc8ccccc89)cc7)c7ccccc7-c7ccccc7)cc6)cc5c4c3o2)cc1 ; c1ccc(-c2nc3ccc4oc5ccc(N(c6ccc(-c7ccccc7-c7ccccc7)cc6)c6ccc7c(c6)oc6ccccc67)cc5c4c3o2)cc1 ; c1ccc(-c2nc3ccc4oc5ccc(N(c6ccc(-c7ccccc7-c7ccccc7)cc6)c6ccc7oc8ccccc8c7c6)cc5c4c3o2)cc1]

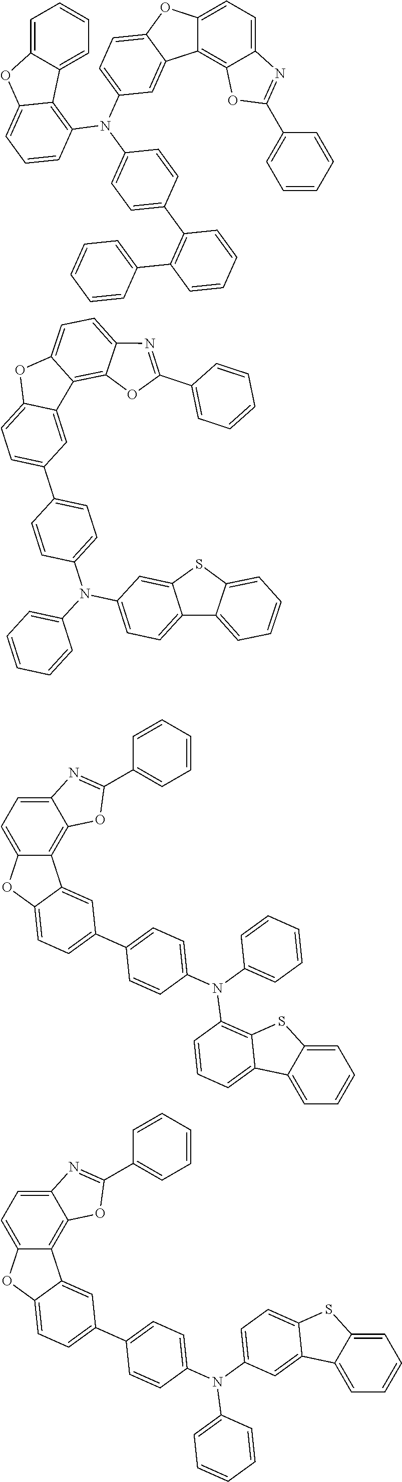

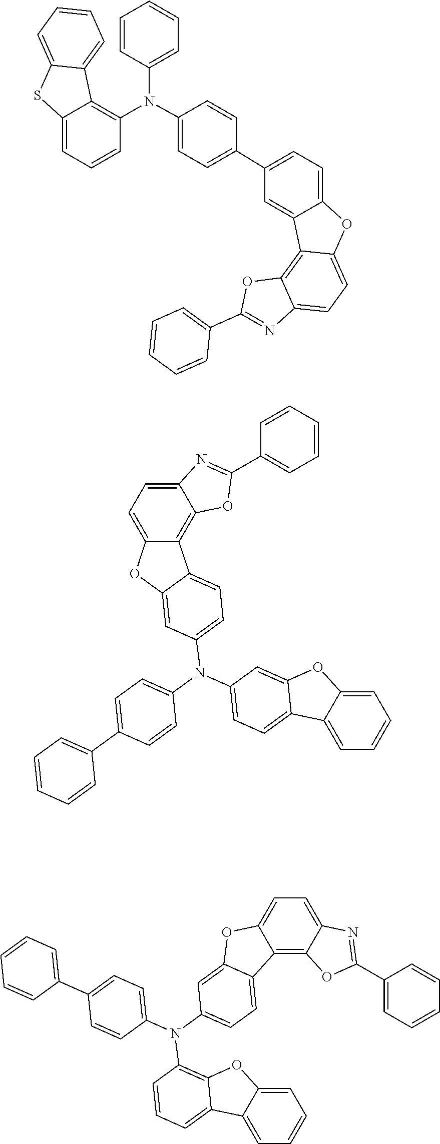 [SMILES: c1ccc(-c2nc3ccc4oc5ccc(-c6ccc(N(c7ccccc7)c7cccc8sc9ccccc9c78)cc6)cc5c4c3o2)cc1 ; c1ccc(-c2ccc(N(c3ccc4c(c3)oc3ccccc34)c3ccc4c(c3)oc3ccc5nc(-c6ccccc6)oc5c34)cc2)cc1 ; c1ccc(-c2ccc(N(c3ccc4c(c3)oc3ccc5nc(-c6ccccc6)oc5c34)c3cccc4c3oc3ccccc34)cc2)cc1]

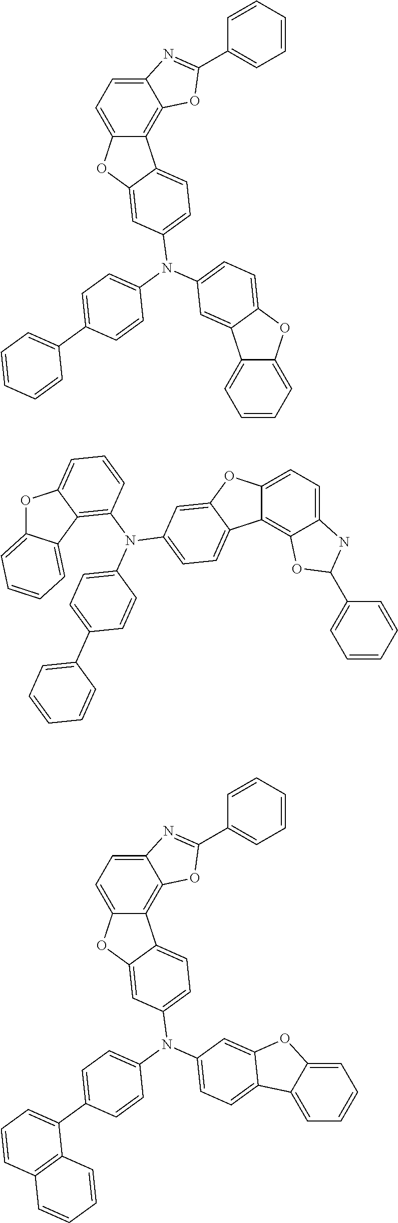

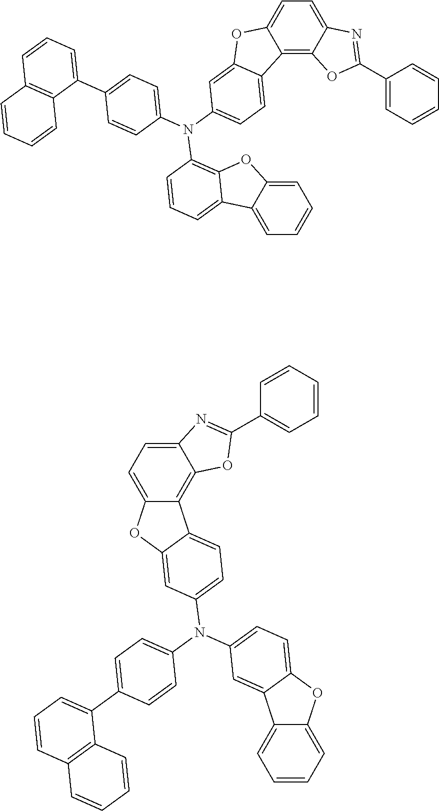 [SMILES: c1ccc(-c2nc3ccc4oc5cc(N(c6ccc(-c7cccc8ccccc78)cc6)c6cccc7c6oc6ccccc67)ccc5c4c3o2)cc1 ; c1ccc(-c2nc3ccc4oc5cc(N(c6ccc(-c7cccc8ccccc78)cc6)c6ccc7oc8ccccc8c7c6)ccc5c4c3o2)cc1]

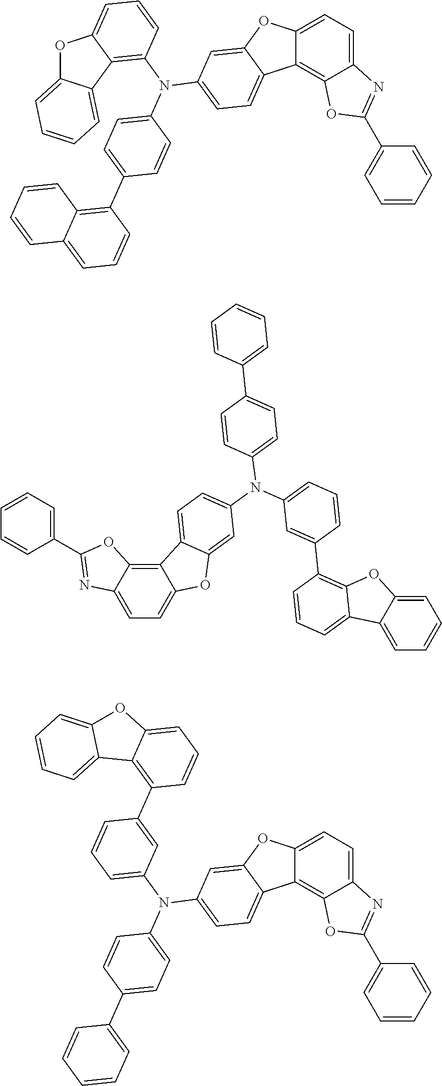 [SMILES: c1ccc(-c2nc3ccc4oc5cc(N(c6ccc(-c7cccc8ccccc78)cc6)c6cccc7oc8ccccc8c67)ccc5c4c3o2)cc1 ; c1ccc(-c2ccc(N(c3cccc(-c4cccc5c4oc4ccccc45)c3)c3ccc4c(c3)oc3ccc5nc(-c6ccccc6)oc5c34)cc2)cc1 ; c1ccc(-c2ccc(N(c3cccc(-c4cccc5oc6ccccc6c45)c3)c3ccc4c(c3)oc3ccc5nc(-c6ccccc6)oc5c34)cc2)cc1]

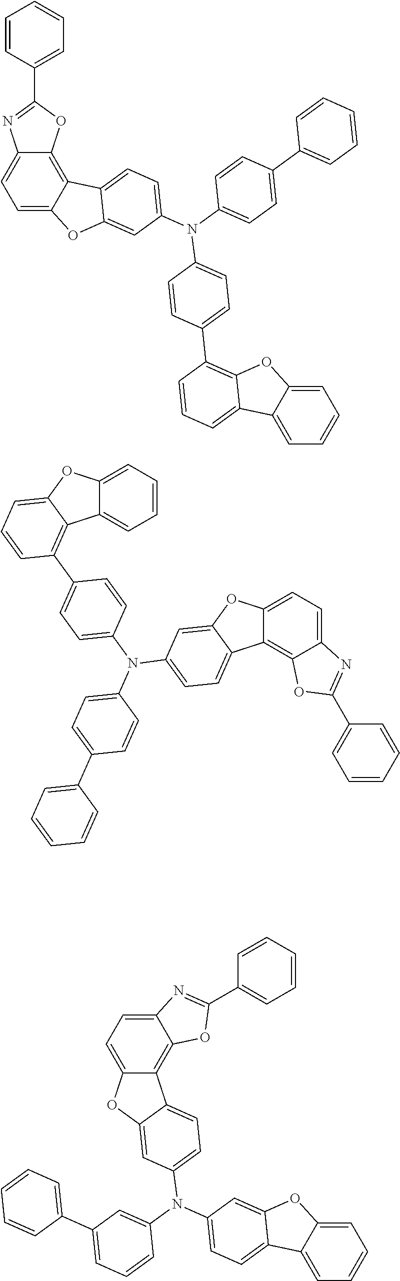 [SMILES: c1ccc(-c2ccc(N(c3ccc(-c4cccc5c4oc4ccccc45)cc3)c3ccc4c(c3)oc3ccc5nc(-c6ccccc6)oc5c34)cc2)cc1 ; c1ccc(-c2ccc(N(c3ccc(-c4cccc5oc6ccccc6c45)cc3)c3ccc4c(c3)oc3ccc5nc(-c6ccccc6)oc5c34)cc2)cc1 ; c1ccc(-c2cccc(N(c3ccc4c(c3)oc3ccccc34)c3ccc4c(c3)oc3ccc5nc(-c6ccccc6)oc5c34)c2)cc1]

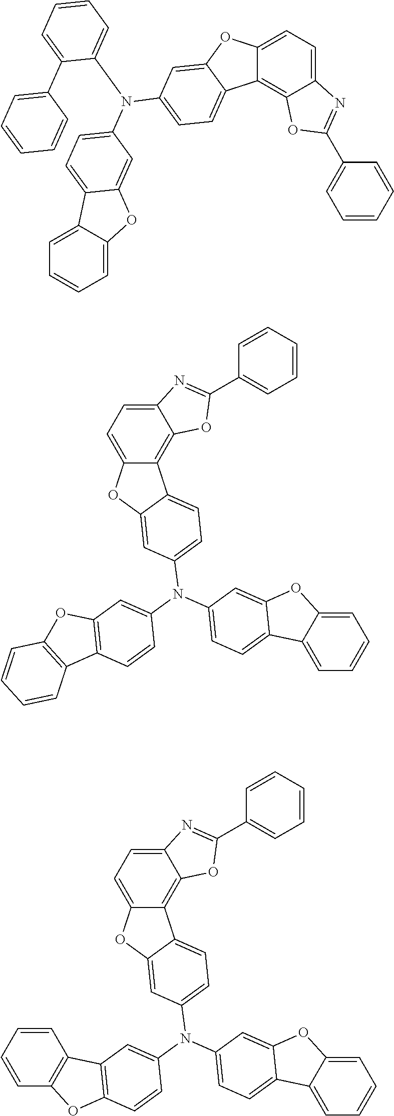 [SMILES: c1ccc(-c2nc3ccc4oc5cc(N(c6ccc7c(c6)oc6ccccc67)c6ccccc6-c6ccccc6)ccc5c4c3o2)cc1 ; c1ccc(-c2nc3ccc4oc5cc(N(c6ccc7c(c6)oc6ccccc67)c6ccc7c(c6)oc6ccccc67)ccc5c4c3o2)cc1 ; c1ccc(-c2nc3ccc4oc5cc(N(c6ccc7c(c6)oc6ccccc67)c6ccc7oc8ccccc8c7c6)ccc5c4c3o2)cc1]

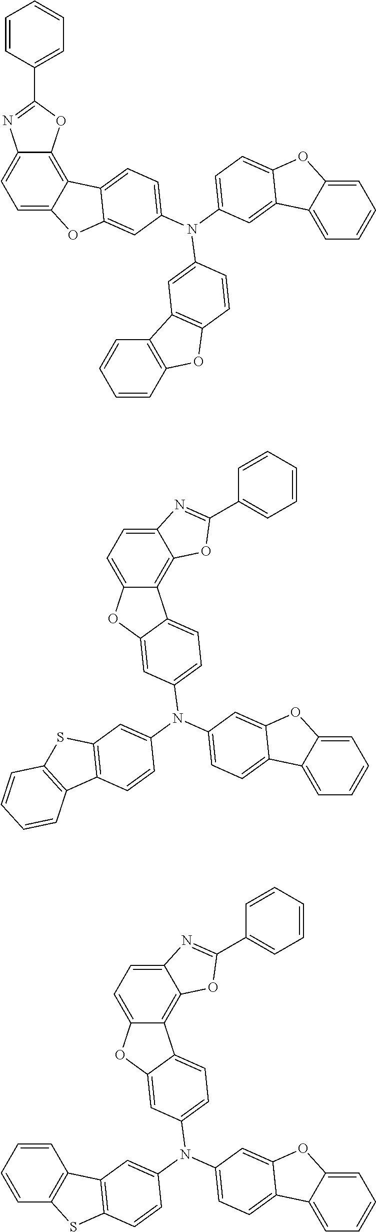 [SMILES: c1ccc(-c2nc3ccc4oc5cc(N(c6ccc7oc8ccccc8c7c6)c6ccc7oc8ccccc8c7c6)ccc5c4c3o2)cc1 ; c1ccc(-c2nc3ccc4oc5cc(N(c6ccc7c(c6)oc6ccccc67)c6ccc7c(c6)sc6ccccc67)ccc5c4c3o2)cc1 ; c1ccc(-c2nc3ccc4oc5cc(N(c6ccc7c(c6)oc6ccccc67)c6ccc7sc8ccccc8c7c6)ccc5c4c3o2)cc1]

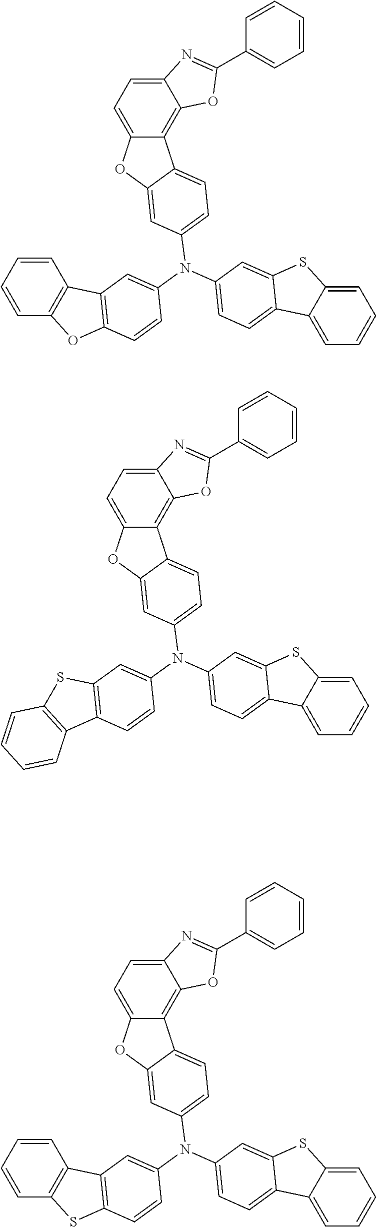 [SMILES: c1ccc(-c2nc3ccc4oc5cc(N(c6ccc7c(c6)sc6ccccc67)c6ccc7oc8ccccc8c7c6)ccc5c4c3o2)cc1 ; c1ccc(-c2nc3ccc4oc5cc(N(c6ccc7c(c6)sc6ccccc67)c6ccc7c(c6)sc6ccccc67)ccc5c4c3o2)cc1 ; c1ccc(-c2nc3ccc4oc5cc(N(c6ccc7c(c6)sc6ccccc67)c6ccc7sc8ccccc8c7c6)ccc5c4c3o2)cc1]

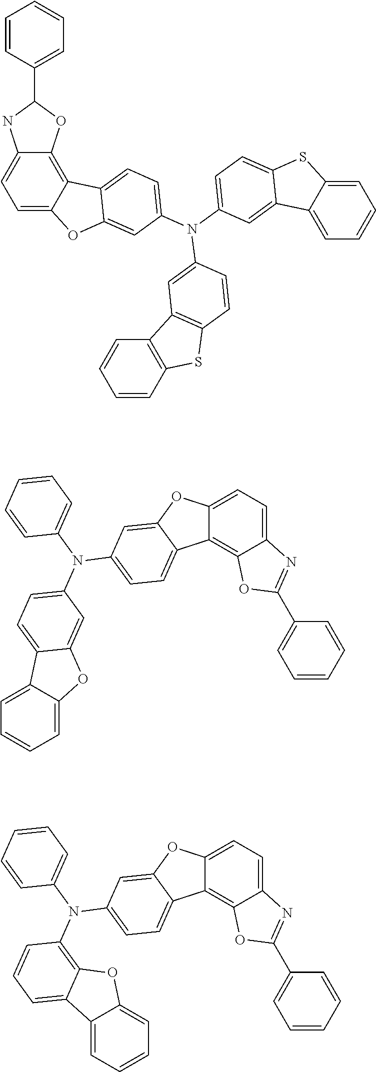 [SMILES: c1ccc(C2Nc3ccc4oc5cc(N(c6ccc7sc8ccccc8c7c6)c6ccc7sc8ccccc8c7c6)ccc5c4c3O2)cc1 ; c1ccc(-c2nc3ccc4oc5cc(N(c6ccccc6)c6ccc7c(c6)oc6ccccc67)ccc5c4c3o2)cc1 ; c1ccc(-c2nc3ccc4oc5cc(N(c6ccccc6)c6cccc7c6oc6ccccc67)ccc5c4c3o2)cc1]

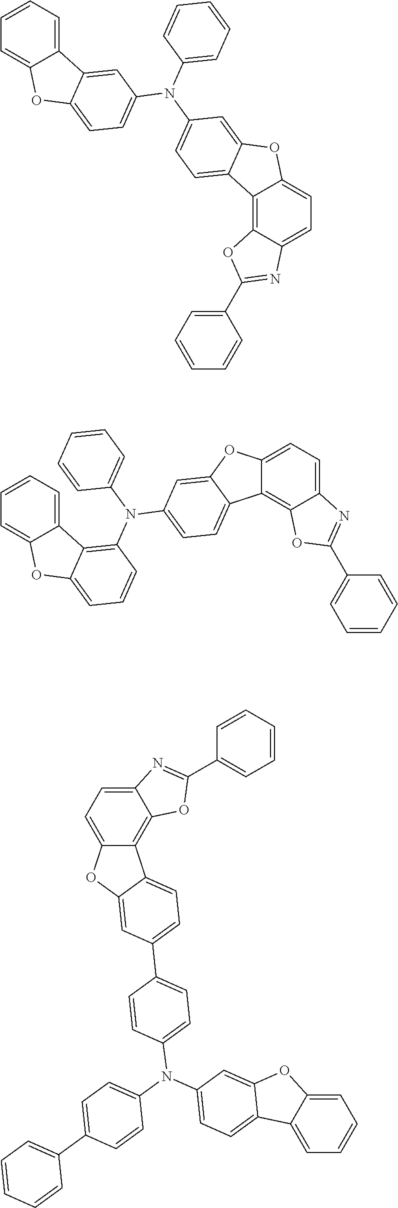 [SMILES: c1ccc(-c2nc3ccc4oc5cc(N(c6ccccc6)c6ccc7oc8ccccc8c7c6)ccc5c4c3o2)cc1 ; c1ccc(-c2nc3ccc4oc5cc(N(c6ccccc6)c6cccc7oc8ccccc8c67)ccc5c4c3o2)cc1 ; c1ccc(-c2ccc(N(c3ccc(-c4ccc5c(c4)oc4ccc6nc(-c7ccccc7)oc6c45)cc3)c3ccc4c(c3)oc3ccccc34)cc2)cc1]

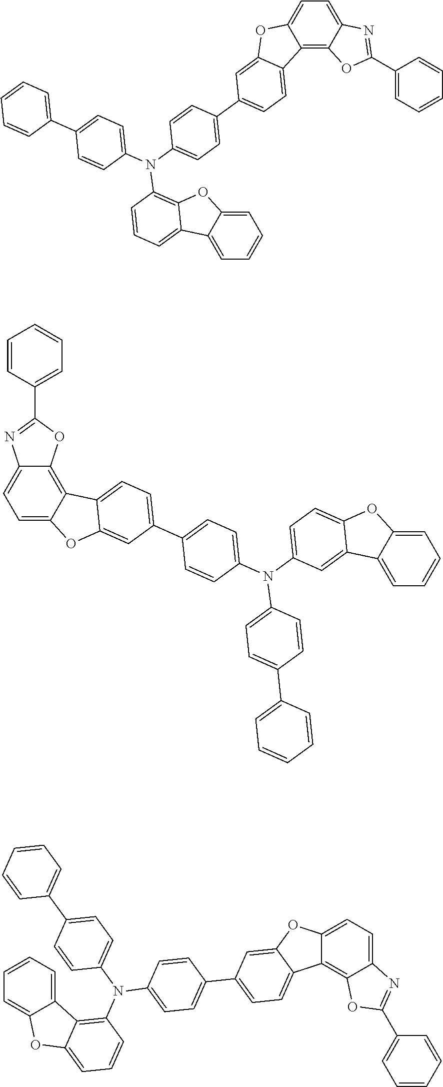 [SMILES: c1ccc(-c2ccc(N(c3ccc(-c4ccc5c(c4)oc4ccc6nc(-c7ccccc7)oc6c45)cc3)c3cccc4c3oc3ccccc34)cc2)cc1 ; c1ccc(-c2ccc(N(c3ccc(-c4ccc5c(c4)oc4ccc6nc(-c7ccccc7)oc6c45)cc3)c3ccc4oc5ccccc5c4c3)cc2)cc1 ; c1ccc(-c2ccc(N(c3ccc(-c4ccc5c(c4)oc4ccc6nc(-c7ccccc7)oc6c45)cc3)c3cccc4oc5ccccc5c34)cc2)cc1]

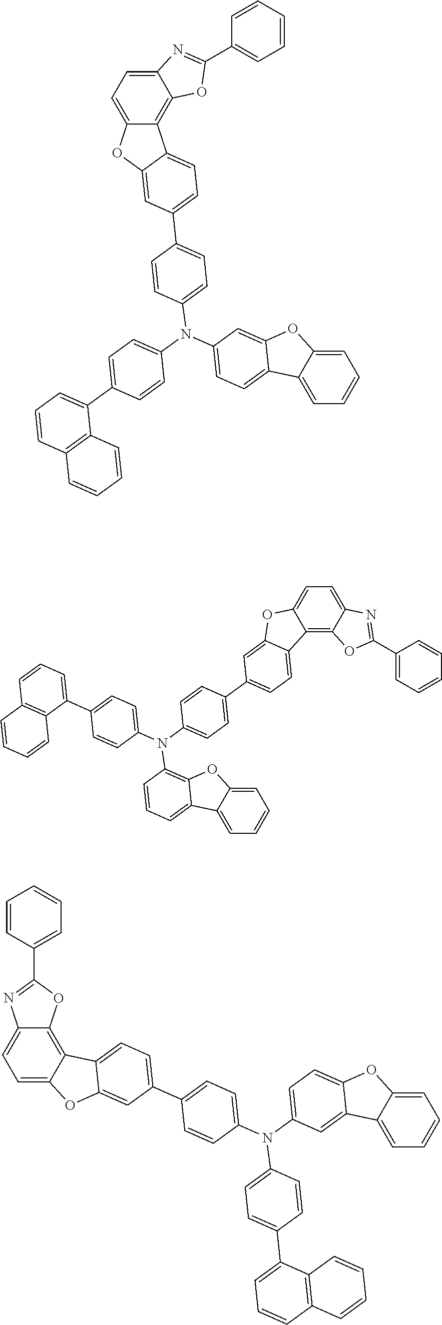 [SMILES: c1ccc(-c2nc3ccc4oc5cc(-c6ccc(N(c7ccc(-c8cccc9ccccc89)cc7)c7ccc8c(c7)oc7ccccc78)cc6)ccc5c4c3o2)cc1 ; c1ccc(-c2nc3ccc4oc5cc(-c6ccc(N(c7ccc(-c8cccc9ccccc89)cc7)c7cccc8c7oc7ccccc78)cc6)ccc5c4c3o2)cc1 ; c1ccc(-c2nc3ccc4oc5cc(-c6ccc(N(c7ccc(-c8cccc9ccccc89)cc7)c7ccc8oc9ccccc9c8c7)cc6)ccc5c4c3o2)cc1]

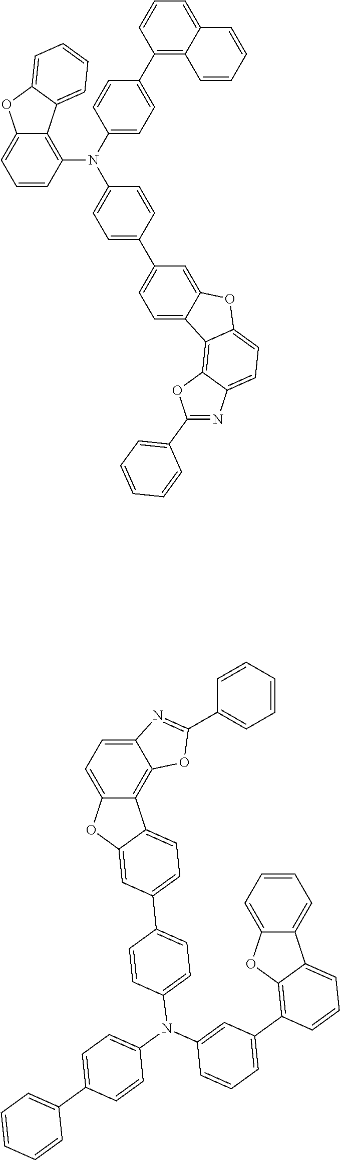 [SMILES: c1ccc(-c2nc3ccc4oc5cc(-c6ccc(N(c7ccc(-c8cccc9ccccc89)cc7)c7cccc8oc9ccccc9c78)cc6)ccc5c4c3o2)cc1 ; c1ccc(-c2ccc(N(c3ccc(-c4ccc5c(c4)oc4ccc6nc(-c7ccccc7)oc6c45)cc3)c3cccc(-c4cccc5c4oc4ccccc45)c3)cc2)cc1]

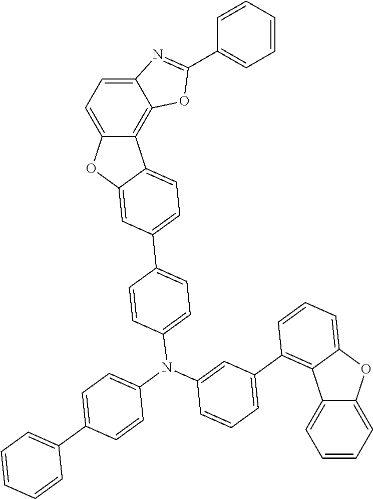 [SMILES: c1ccc(-c2ccc(N(c3ccc(-c4ccc5c(c4)oc4ccc6nc(-c7ccccc7)oc6c45)cc3)c3cccc(-c4cccc5oc6ccccc6c45)c3)cc2)cc1]

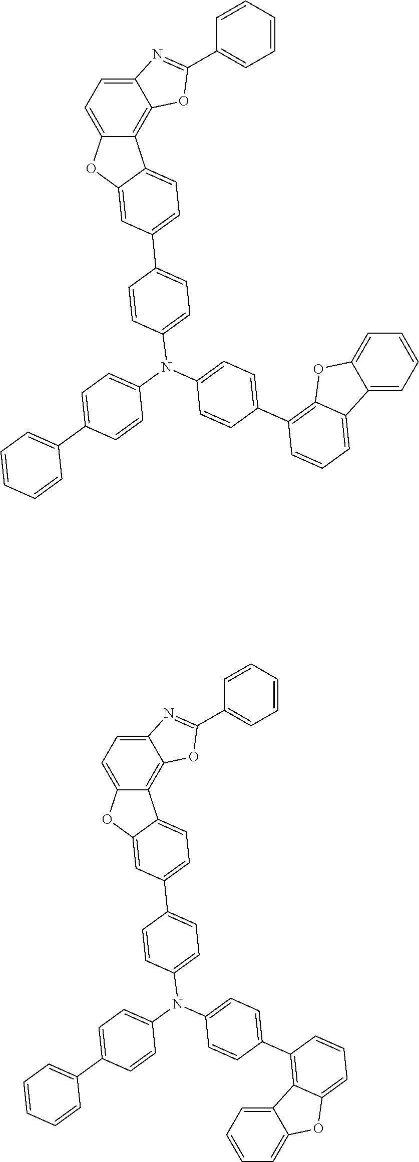 [SMILES: c1ccc(-c2ccc(N(c3ccc(-c4ccc5c(c4)oc4ccc6nc(-c7ccccc7)oc6c45)cc3)c3ccc(-c4cccc5c4oc4ccccc45)cc3)cc2)cc1 ; c1ccc(-c2ccc(N(c3ccc(-c4ccc5c(c4)oc4ccc6nc(-c7ccccc7)oc6c45)cc3)c3ccc(-c4cccc5oc6ccccc6c45)cc3)cc2)cc1]

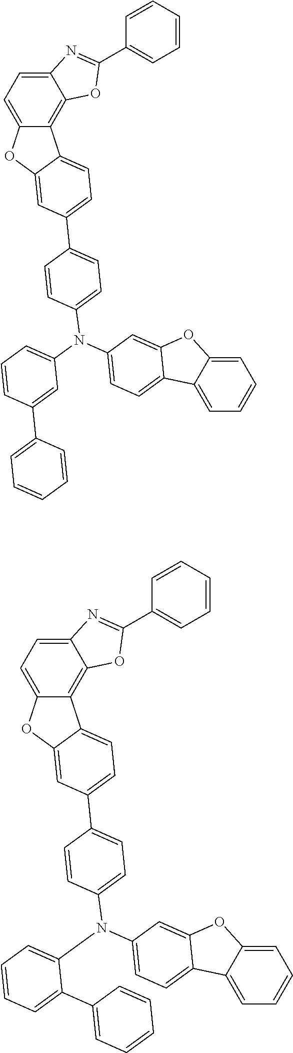 [SMILES: c1ccc(-c2cccc(N(c3ccc(-c4ccc5c(c4)oc4ccc6nc(-c7ccccc7)oc6c45)cc3)c3ccc4c(c3)oc3ccccc34)c2)cc1 ; c1ccc(-c2nc3ccc4oc5cc(-c6ccc(N(c7ccc8c(c7)oc7ccccc78)c7ccccc7-c7ccccc7)cc6)ccc5c4c3o2)cc1]

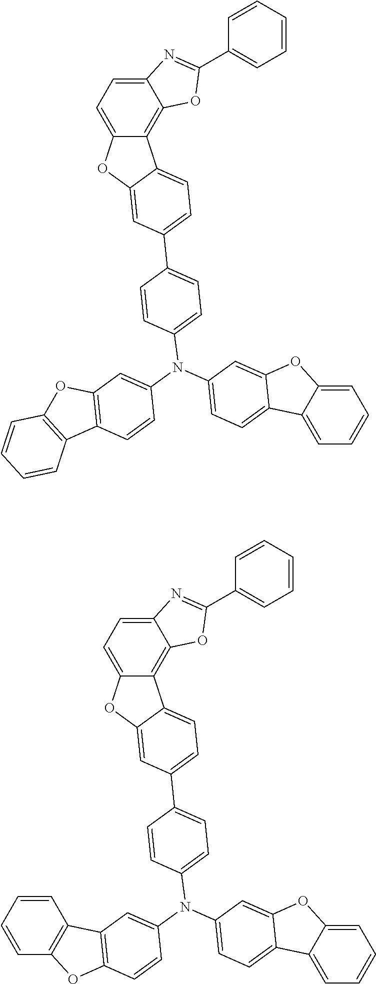 [SMILES: c1ccc(-c2nc3ccc4oc5cc(-c6ccc(N(c7ccc8c(c7)oc7ccccc78)c7ccc8c(c7)oc7ccccc78)cc6)ccc5c4c3o2)cc1 ; c1ccc(-c2nc3ccc4oc5cc(-c6ccc(N(c7ccc8c(c7)oc7ccccc78)c7ccc8oc9ccccc9c8c7)cc6)ccc5c4c3o2)cc1]

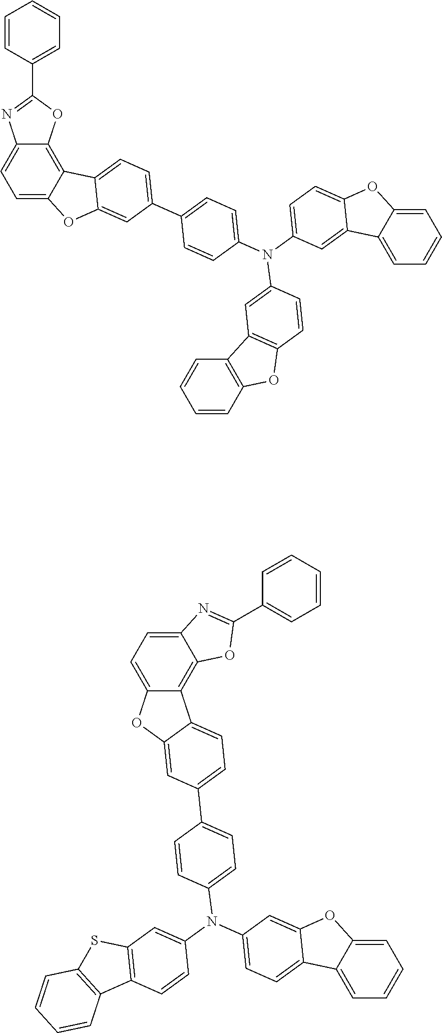 [SMILES: c1ccc(-c2nc3ccc4oc5cc(-c6ccc(N(c7ccc8oc9ccccc9c8c7)c7ccc8oc9ccccc9c8c7)cc6)ccc5c4c3o2)cc1 ; c1ccc(-c2nc3ccc4oc5cc(-c6ccc(N(c7ccc8c(c7)oc7ccccc78)c7ccc8c(c7)sc7ccccc78)cc6)ccc5c4c3o2)cc1]

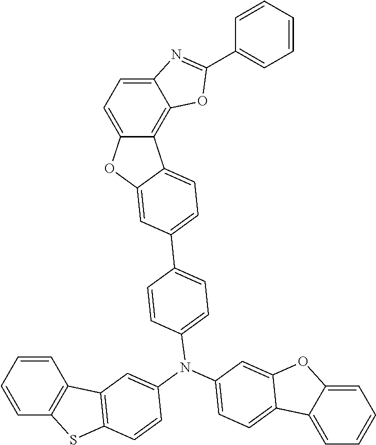 [SMILES: c1ccc(-c2nc3ccc4oc5cc(-c6ccc(N(c7ccc8c(c7)oc7ccccc78)c7ccc8sc9ccccc9c8c7)cc6)ccc5c4c3o2)cc1]

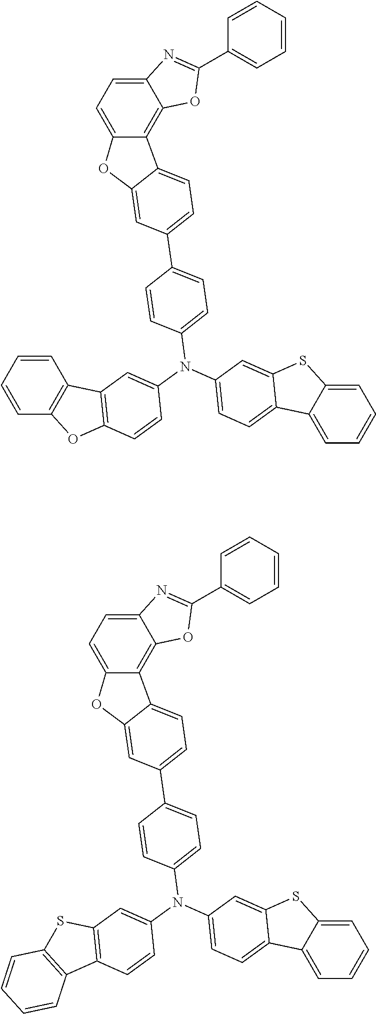 [SMILES: c1ccc(-c2nc3ccc4oc5cc(-c6ccc(N(c7ccc8c(c7)sc7ccccc78)c7ccc8oc9ccccc9c8c7)cc6)ccc5c4c3o2)cc1 ; c1ccc(-c2nc3ccc4oc5cc(-c6ccc(N(c7ccc8c(c7)sc7ccccc78)c7ccc8c(c7)sc7ccccc78)cc6)ccc5c4c3o2)cc1]

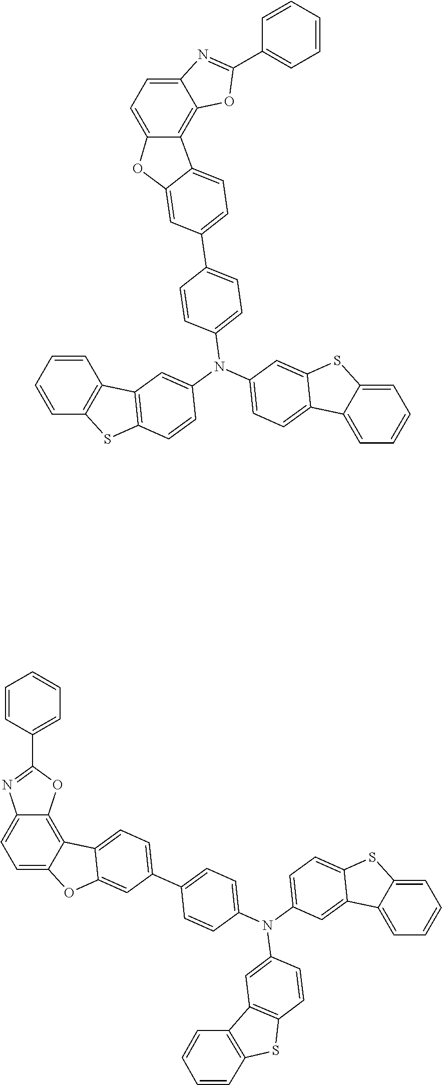 [SMILES: c1ccc(-c2nc3ccc4oc5cc(-c6ccc(N(c7ccc8c(c7)sc7ccccc78)c7ccc8sc9ccccc9c8c7)cc6)ccc5c4c3o2)cc1 ; c1ccc(-c2nc3ccc4oc5cc(-c6ccc(N(c7ccc8sc9ccccc9c8c7)c7ccc8sc9ccccc9c8c7)cc6)ccc5c4c3o2)cc1]

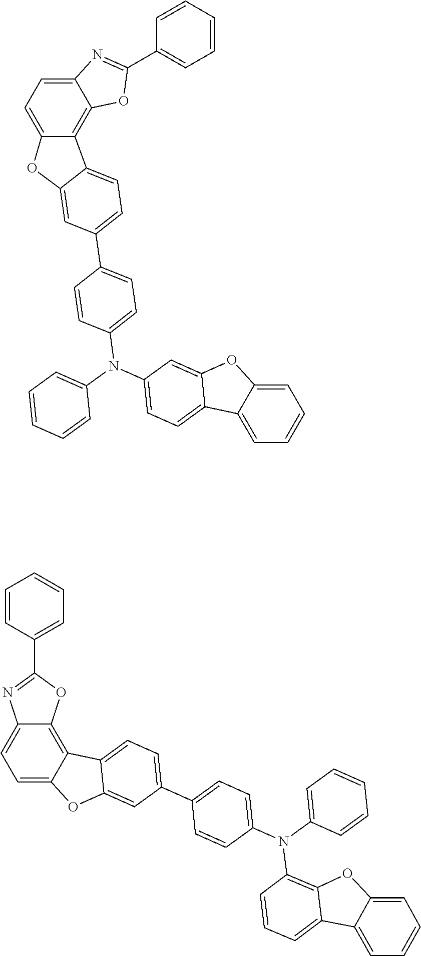 [SMILES: c1ccc(-c2nc3ccc4oc5cc(-c6ccc(N(c7ccccc7)c7ccc8c(c7)oc7ccccc78)cc6)ccc5c4c3o2)cc1 ; c1ccc(-c2nc3ccc4oc5cc(-c6ccc(N(c7ccccc7)c7cccc8c7oc7ccccc78)cc6)ccc5c4c3o2)cc1]

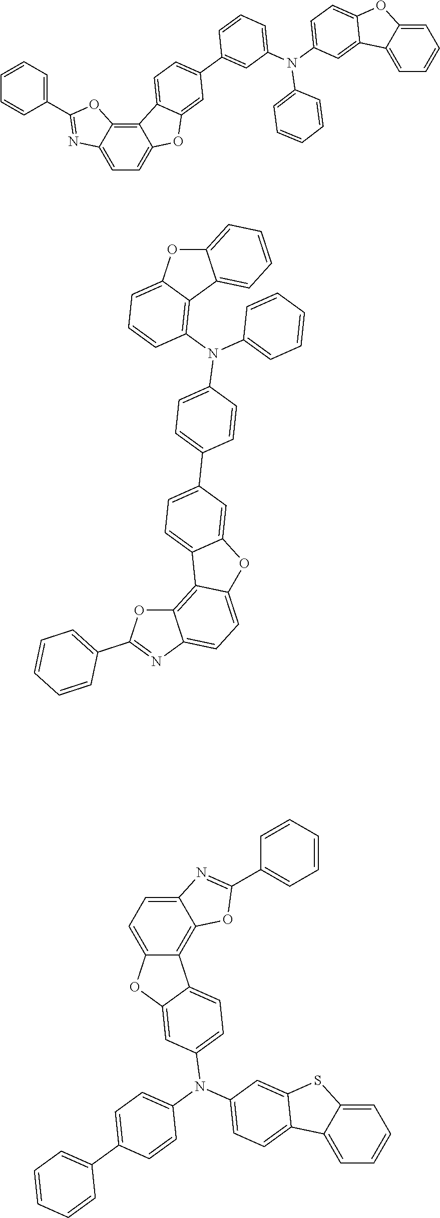 [SMILES: c1ccc(-c2nc3ccc4oc5cc(-c6cccc(N(c7ccccc7)c7ccc8oc9ccccc9c8c7)c6)ccc5c4c3o2)cc1 ; c1ccc(-c2nc3ccc4oc5cc(-c6ccc(N(c7ccccc7)c7cccc8oc9ccccc9c78)cc6)ccc5c4c3o2)cc1 ; c1ccc(-c2ccc(N(c3ccc4c(c3)oc3ccc5nc(-c6ccccc6)oc5c34)c3ccc4c(c3)sc3ccccc34)cc2)cc1]

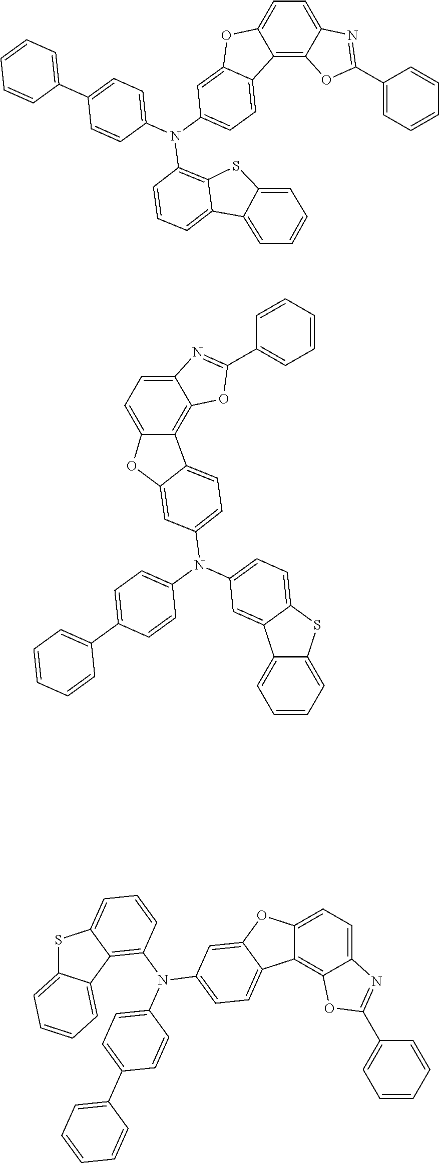 [SMILES: c1ccc(-c2ccc(N(c3ccc4c(c3)oc3ccc5nc(-c6ccccc6)oc5c34)c3cccc4c3sc3ccccc34)cc2)cc1 ; c1ccc(-c2ccc(N(c3ccc4c(c3)oc3ccc5nc(-c6ccccc6)oc5c34)c3ccc4sc5ccccc5c4c3)cc2)cc1 ; c1ccc(-c2ccc(N(c3ccc4c(c3)oc3ccc5nc(-c6ccccc6)oc5c34)c3cccc4sc5ccccc5c34)cc2)cc1]

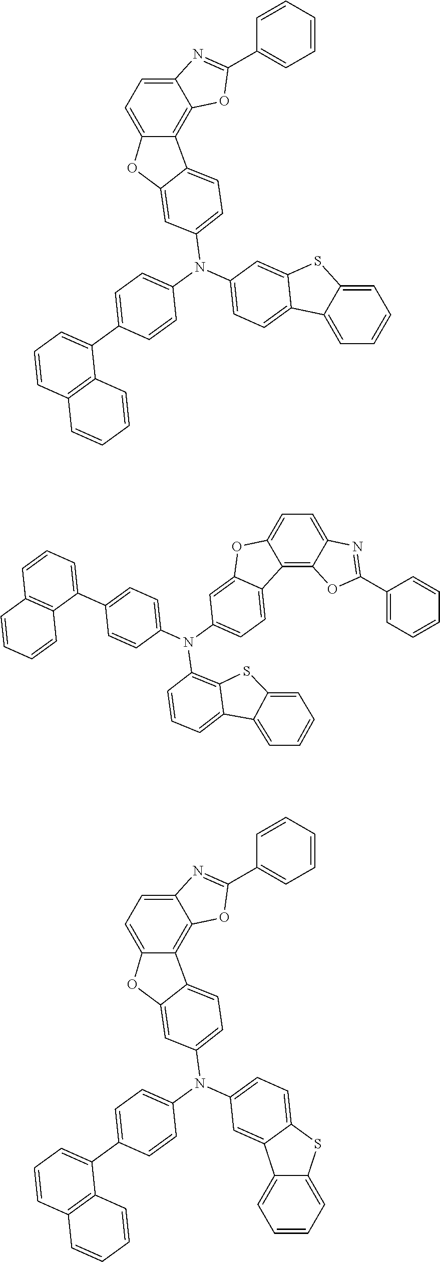 [SMILES: c1ccc(-c2nc3ccc4oc5cc(N(c6ccc(-c7cccc8ccccc78)cc6)c6ccc7c(c6)sc6ccccc67)ccc5c4c3o2)cc1 ; c1ccc(-c2nc3ccc4oc5cc(N(c6ccc(-c7cccc8ccccc78)cc6)c6cccc7c6sc6ccccc67)ccc5c4c3o2)cc1 ; c1ccc(-c2nc3ccc4oc5cc(N(c6ccc(-c7cccc8ccccc78)cc6)c6ccc7sc8ccccc8c7c6)ccc5c4c3o2)cc1]

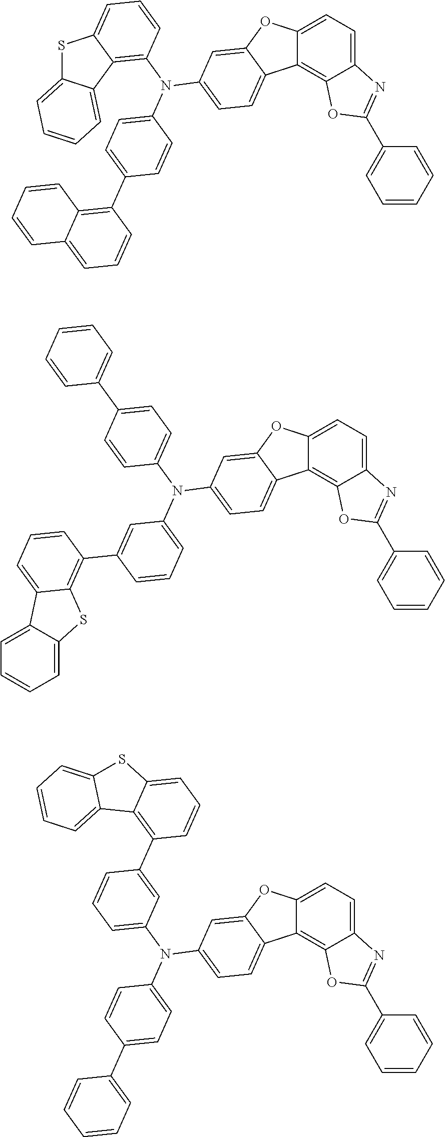 [SMILES: c1ccc(-c2nc3ccc4oc5cc(N(c6ccc(-c7cccc8ccccc78)cc6)c6cccc7sc8ccccc8c67)ccc5c4c3o2)cc1 ; c1ccc(-c2ccc(N(c3cccc(-c4cccc5c4sc4ccccc45)c3)c3ccc4c(c3)oc3ccc5nc(-c6ccccc6)oc5c34)cc2)cc1 ; c1ccc(-c2ccc(N(c3cccc(-c4cccc5sc6ccccc6c45)c3)c3ccc4c(c3)oc3ccc5nc(-c6ccccc6)oc5c34)cc2)cc1]

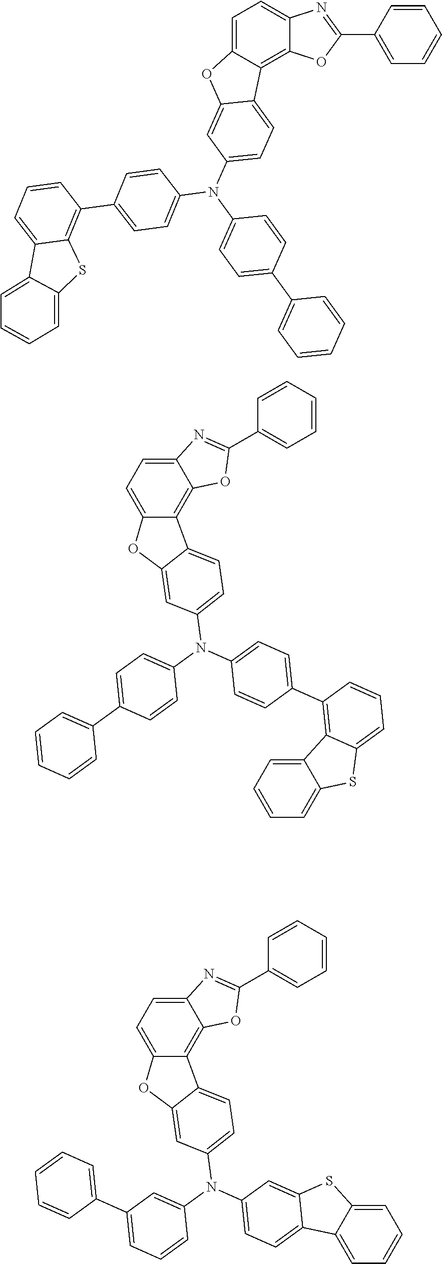

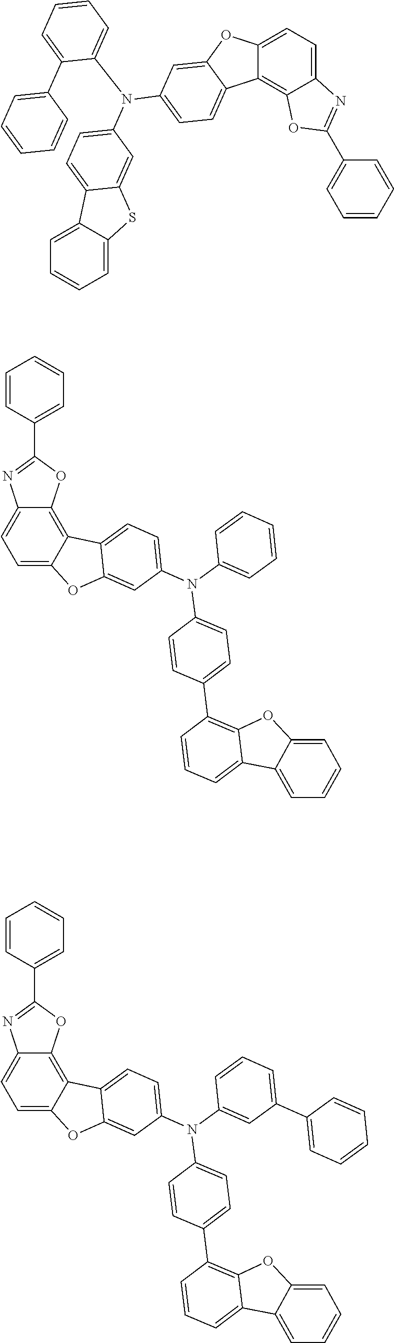 [SMILES: c1ccc(-c2nc3ccc4oc5cc(N(c6ccc7c(c6)sc6ccccc67)c6ccccc6-c6ccccc6)ccc5c4c3o2)cc1 ; c1ccc(-c2nc3ccc4oc5cc(N(c6ccccc6)c6ccc(-c7cccc8c7oc7ccccc78)cc6)ccc5c4c3o2)cc1 ; c1ccc(-c2cccc(N(c3ccc(-c4cccc5c4oc4ccccc45)cc3)c3ccc4c(c3)oc3ccc5nc(-c6ccccc6)oc5c34)c2)cc1]

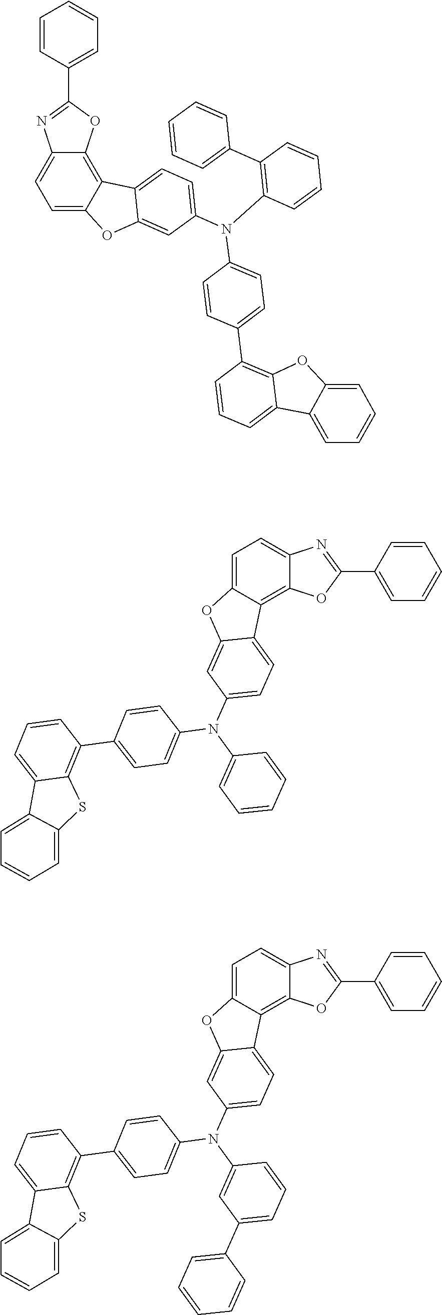 [SMILES: c1ccc(-c2nc3ccc4oc5cc(N(c6ccc(-c7cccc8c7oc7ccccc78)cc6)c6ccccc6-c6ccccc6)ccc5c4c3o2)cc1 ; c1ccc(-c2nc3ccc4oc5cc(N(c6ccccc6)c6ccc(-c7cccc8c7sc7ccccc78)cc6)ccc5c4c3o2)cc1 ; c1ccc(-c2cccc(N(c3ccc(-c4cccc5c4sc4ccccc45)cc3)c3ccc4c(c3)oc3ccc5nc(-c6ccccc6)oc5c34)c2)cc1]

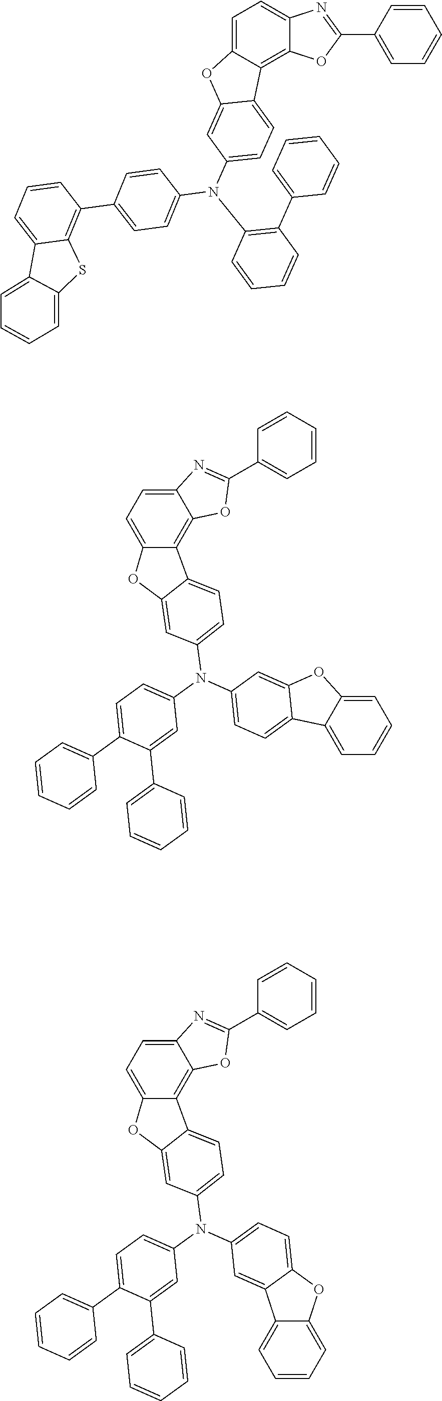 [SMILES: c1ccc(-c2nc3ccc4oc5cc(N(c6ccc(-c7cccc8c7sc7ccccc78)cc6)c6ccccc6-c6ccccc6)ccc5c4c3o2)cc1 ; c1ccc(-c2nc3ccc4oc5cc(N(c6ccc(-c7ccccc7)c(-c7ccccc7)c6)c6ccc7c(c6)oc6ccccc67)ccc5c4c3o2)cc1 ; c1ccc(-c2nc3ccc4oc5cc(N(c6ccc(-c7ccccc7)c(-c7ccccc7)c6)c6ccc7oc8ccccc8c7c6)ccc5c4c3o2)cc1]

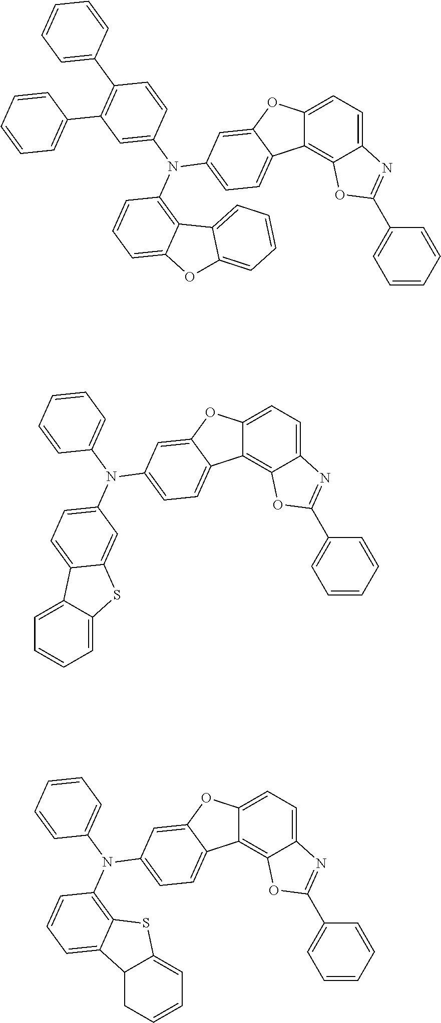 [SMILES: c1ccc(-c2nc3ccc4oc5cc(N(c6ccc(-c7ccccc7)c(-c7ccccc7)c6)c6cccc7oc8ccccc8c67)ccc5c4c3o2)cc1 ; c1ccc(-c2nc3ccc4oc5cc(N(c6ccccc6)c6ccc7c(c6)sc6ccccc67)ccc5c4c3o2)cc1 ; C1=CCC2C(=C1)Sc1c2cccc1N(c1ccccc1)c1ccc2c(c1)oc1ccc3nc(-c4ccccc4)oc3c12]

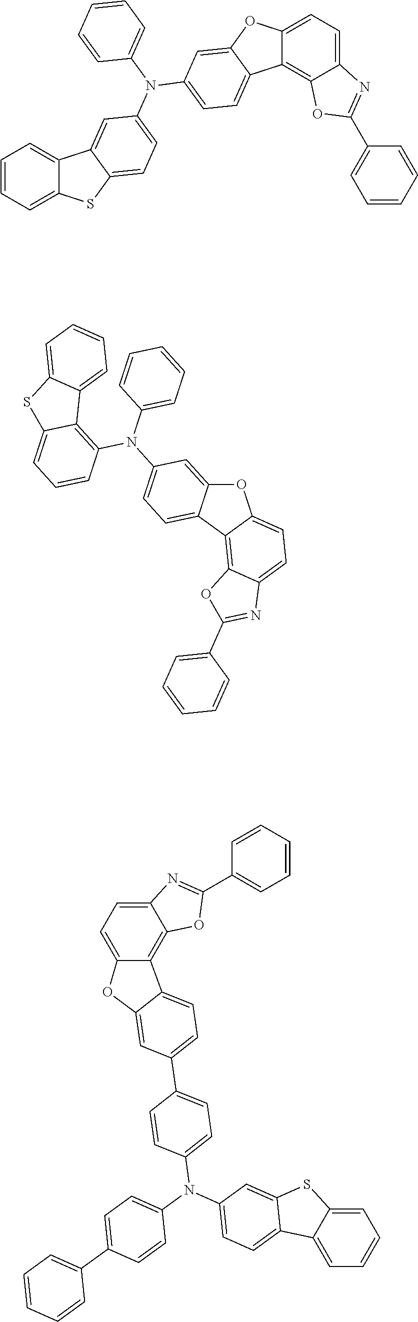 [SMILES: c1ccc(-c2nc3ccc4oc5cc(N(c6ccccc6)c6ccc7sc8ccccc8c7c6)ccc5c4c3o2)cc1 ; c1ccc(-c2nc3ccc4oc5cc(N(c6ccccc6)c6cccc7sc8ccccc8c67)ccc5c4c3o2)cc1 ; c1ccc(-c2ccc(N(c3ccc(-c4ccc5c(c4)oc4ccc6nc(-c7ccccc7)oc6c45)cc3)c3ccc4c(c3)sc3ccccc34)cc2)cc1]

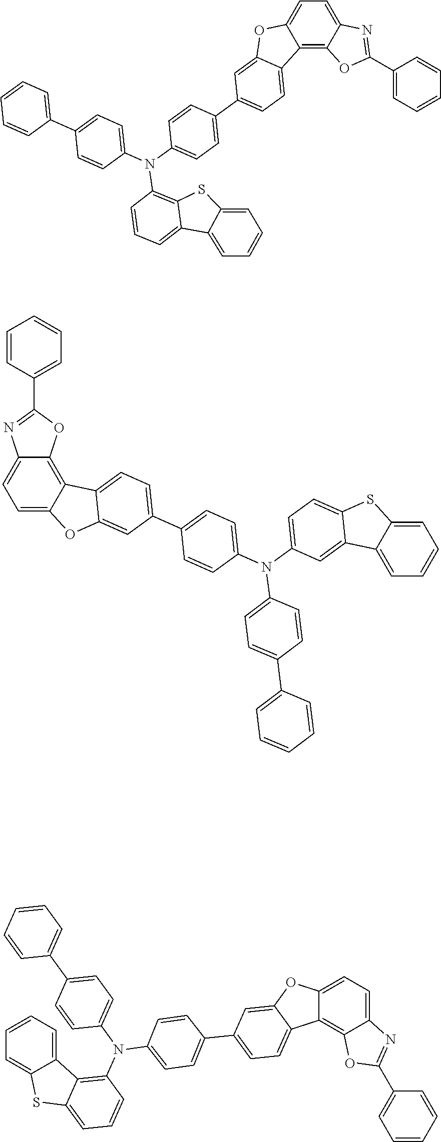 [SMILES: c1ccc(-c2ccc(N(c3ccc(-c4ccc5c(c4)oc4ccc6nc(-c7ccccc7)oc6c45)cc3)c3cccc4c3sc3ccccc34)cc2)cc1 ; c1ccc(-c2ccc(N(c3ccc(-c4ccc5c(c4)oc4ccc6nc(-c7ccccc7)oc6c45)cc3)c3ccc4sc5ccccc5c4c3)cc2)cc1 ; c1ccc(-c2ccc(N(c3ccc(-c4ccc5c(c4)oc4ccc6nc(-c7ccccc7)oc6c45)cc3)c3cccc4sc5ccccc5c34)cc2)cc1]

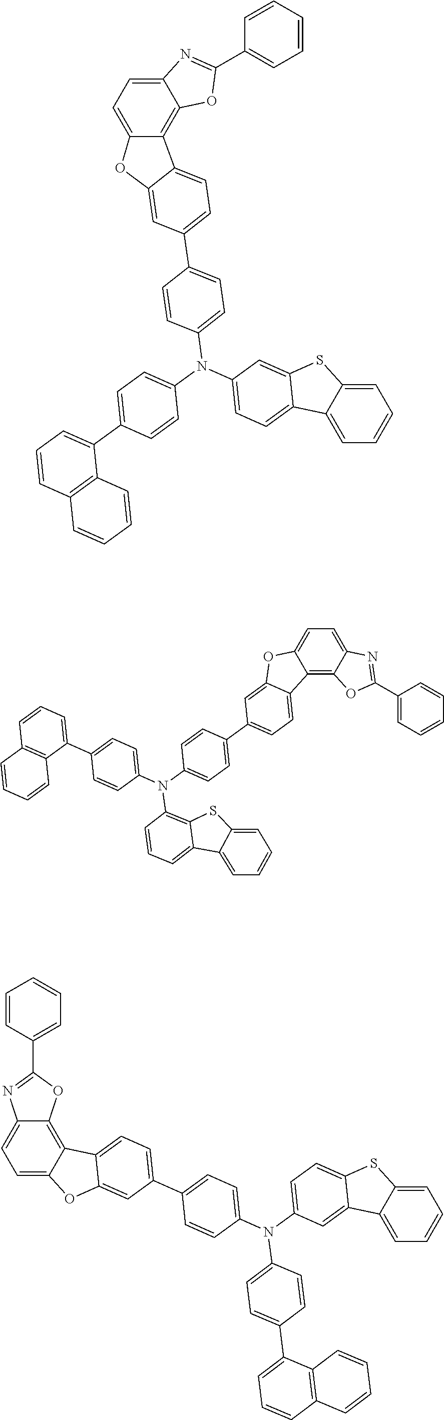 [SMILES: c1ccc(-c2nc3ccc4oc5cc(-c6ccc(N(c7ccc(-c8cccc9ccccc89)cc7)c7ccc8c(c7)sc7ccccc78)cc6)ccc5c4c3o2)cc1 ; c1ccc(-c2nc3ccc4oc5cc(-c6ccc(N(c7ccc(-c8cccc9ccccc89)cc7)c7cccc8c7sc7ccccc78)cc6)ccc5c4c3o2)cc1 ; c1ccc(-c2nc3ccc4oc5cc(-c6ccc(N(c7ccc(-c8cccc9ccccc89)cc7)c7ccc8sc9ccccc9c8c7)cc6)ccc5c4c3o2)cc1]

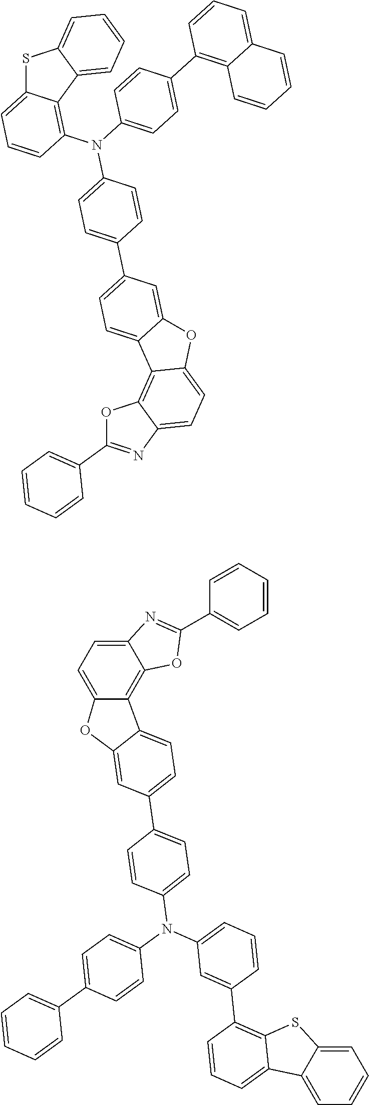 [SMILES: c1ccc(-c2nc3ccc4oc5cc(-c6ccc(N(c7ccc(-c8cccc9ccccc89)cc7)c7cccc8sc9ccccc9c78)cc6)ccc5c4c3o2)cc1 ; c1ccc(-c2ccc(N(c3ccc(-c4ccc5c(c4)oc4ccc6nc(-c7ccccc7)oc6c45)cc3)c3cccc(-c4cccc5c4sc4ccccc45)c3)cc2)cc1]

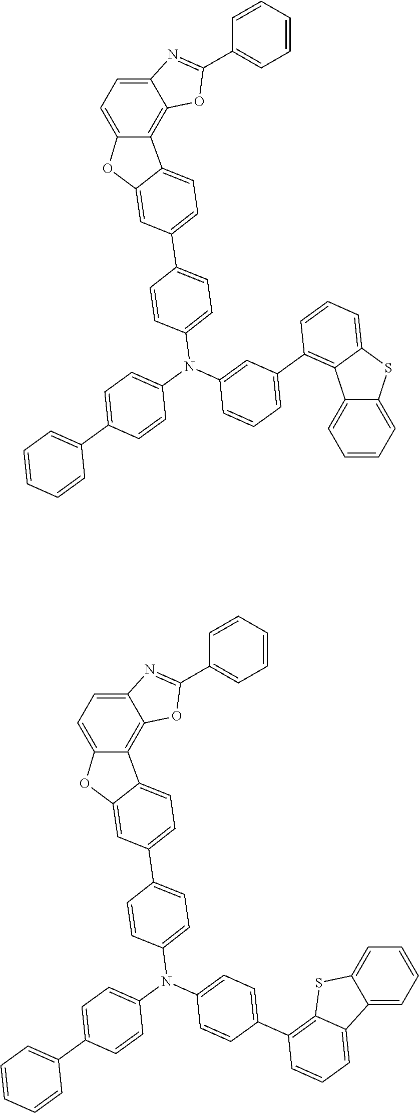 [SMILES: c1ccc(-c2ccc(N(c3ccc(-c4ccc5c(c4)oc4ccc6nc(-c7ccccc7)oc6c45)cc3)c3cccc(-c4cccc5sc6ccccc6c45)c3)cc2)cc1 ; c1ccc(-c2ccc(N(c3ccc(-c4ccc5c(c4)oc4ccc6nc(-c7ccccc7)oc6c45)cc3)c3ccc(-c4cccc5c4sc4ccccc45)cc3)cc2)cc1]

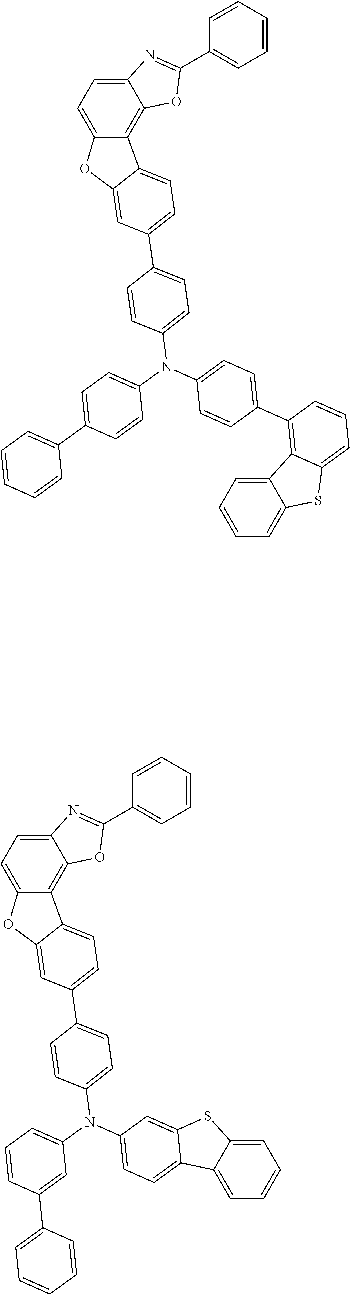 [SMILES: c1ccc(-c2ccc(N(c3ccc(-c4ccc5c(c4)oc4ccc6nc(-c7ccccc7)oc6c45)cc3)c3ccc(-c4cccc5sc6ccccc6c45)cc3)cc2)cc1 ; c1ccc(-c2cccc(N(c3ccc(-c4ccc5c(c4)oc4ccc6nc(-c7ccccc7)oc6c45)cc3)c3ccc4c(c3)sc3ccccc34)c2)cc1]

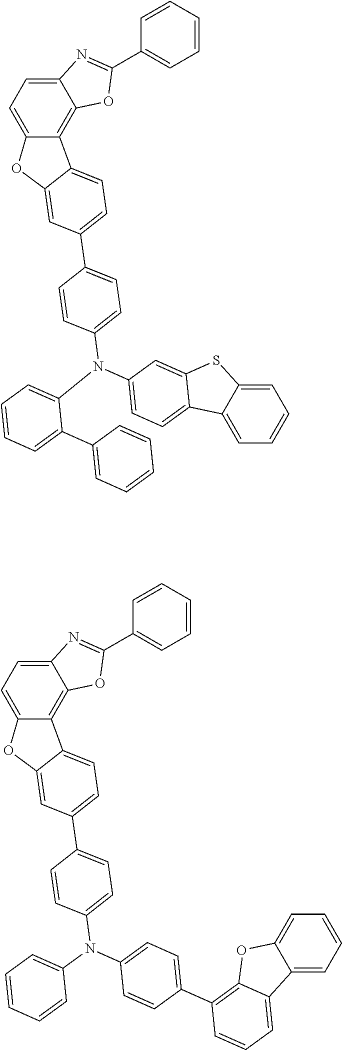 [SMILES: c1ccc(-c2nc3ccc4oc5cc(-c6ccc(N(c7ccc8c(c7)sc7ccccc78)c7ccccc7-c7ccccc7)cc6)ccc5c4c3o2)cc1 ; c1ccc(-c2nc3ccc4oc5cc(-c6ccc(N(c7ccccc7)c7ccc(-c8cccc9c8oc8ccccc89)cc7)cc6)ccc5c4c3o2)cc1]

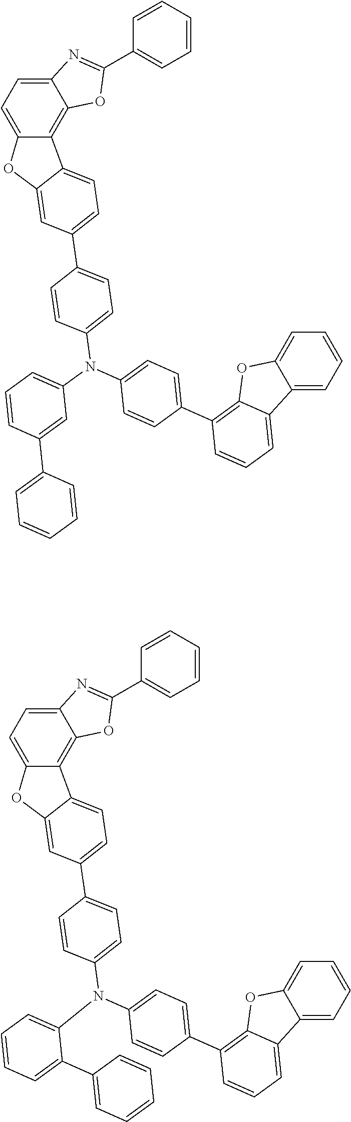 [SMILES: c1ccc(-c2cccc(N(c3ccc(-c4ccc5c(c4)oc4ccc6nc(-c7ccccc7)oc6c45)cc3)c3ccc(-c4cccc5c4oc4ccccc45)cc3)c2)cc1 ; c1ccc(-c2nc3ccc4oc5cc(-c6ccc(N(c7ccc(-c8cccc9c8oc8ccccc89)cc7)c7ccccc7-c7ccccc7)cc6)ccc5c4c3o2)cc1]

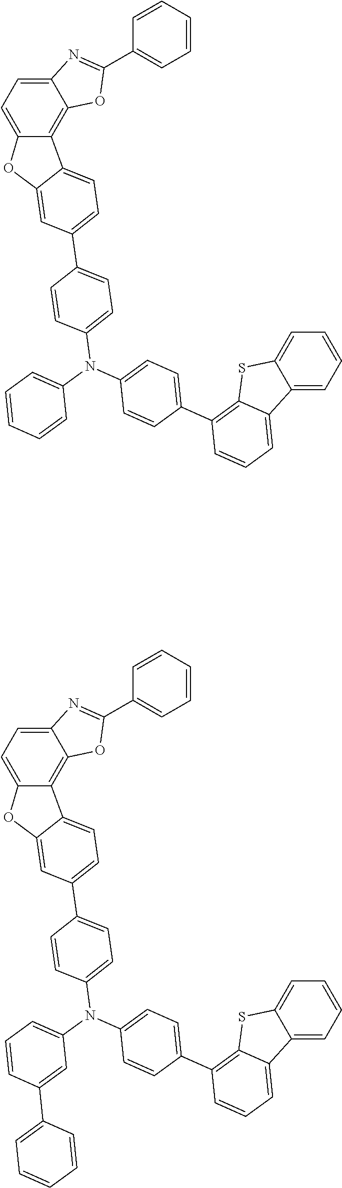 [SMILES: c1ccc(-c2nc3ccc4oc5cc(-c6ccc(N(c7ccccc7)c7ccc(-c8cccc9c8sc8ccccc89)cc7)cc6)ccc5c4c3o2)cc1 ; c1ccc(-c2cccc(N(c3ccc(-c4ccc5c(c4)oc4ccc6nc(-c7ccccc7)oc6c45)cc3)c3ccc(-c4cccc5c4sc4ccccc45)cc3)c2)cc1]

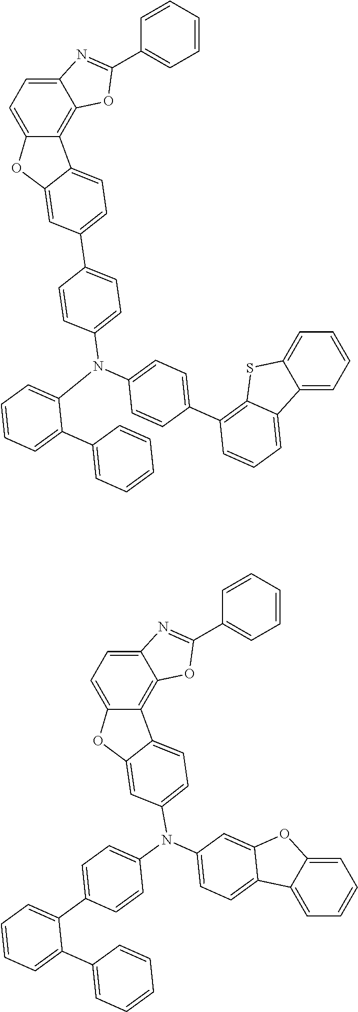 [SMILES: c1ccc(-c2nc3ccc4oc5cc(-c6ccc(N(c7ccc(-c8cccc9c8sc8ccccc89)cc7)c7ccccc7-c7ccccc7)cc6)ccc5c4c3o2)cc1 ; c1ccc(-c2nc3ccc4oc5cc(N(c6ccc(-c7ccccc7-c7ccccc7)cc6)c6ccc7c(c6)oc6ccccc67)ccc5c4c3o2)cc1]

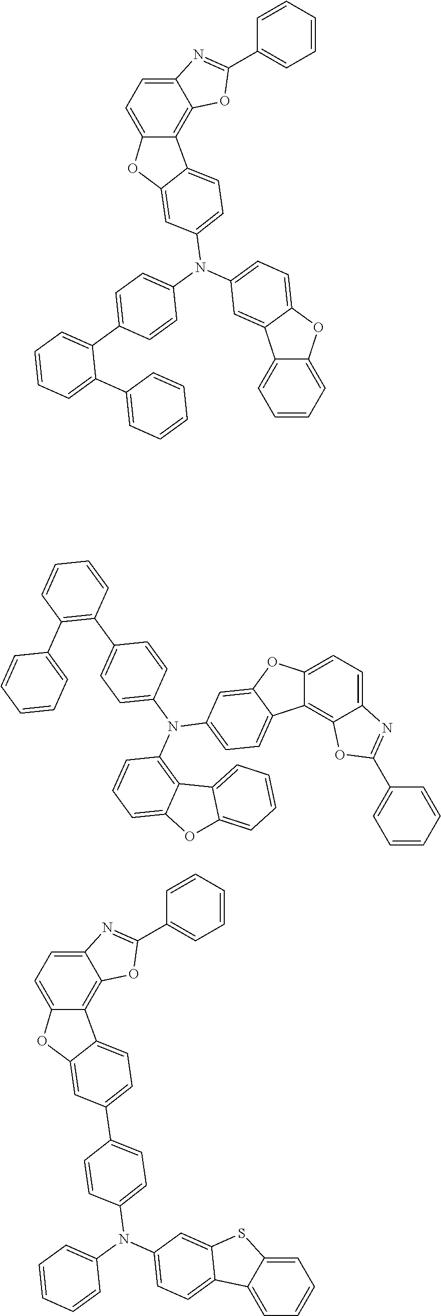

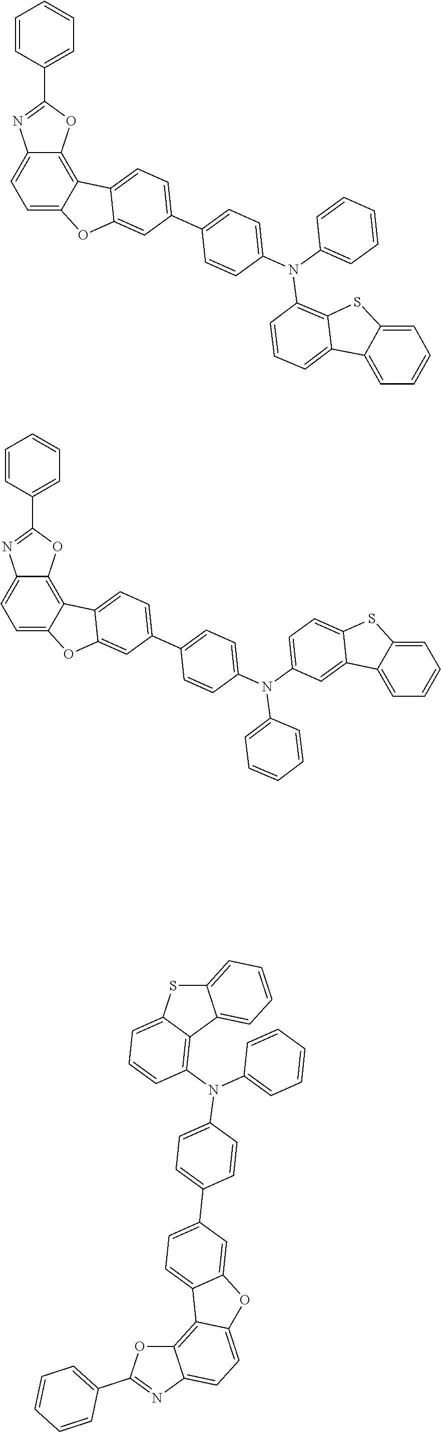 [SMILES: c1ccc(-c2nc3ccc4oc5cc(-c6ccc(N(c7ccccc7)c7cccc8c7sc7ccccc78)cc6)ccc5c4c3o2)cc1 ; c1ccc(-c2nc3ccc4oc5cc(-c6ccc(N(c7ccccc7)c7ccc8sc9ccccc9c8c7)cc6)ccc5c4c3o2)cc1 ; c1ccc(-c2nc3ccc4oc5cc(-c6ccc(N(c7ccccc7)c7cccc8sc9ccccc9c78)cc6)ccc5c4c3o2)cc1]

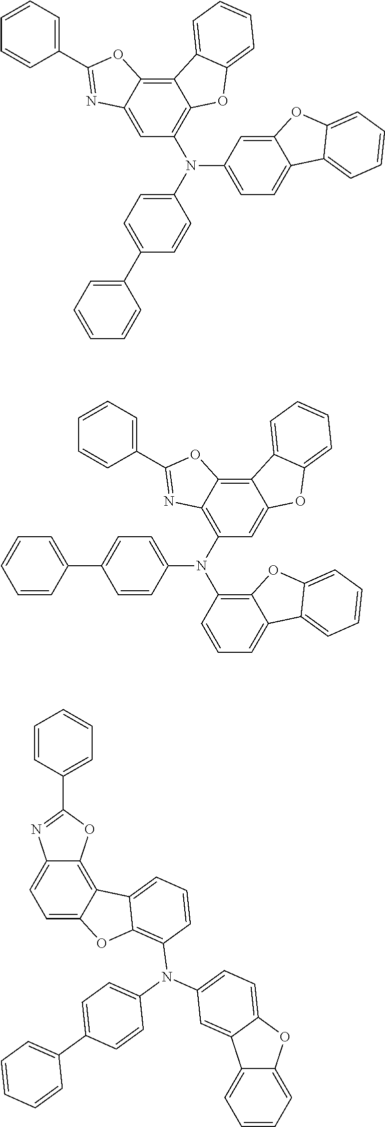 [SMILES: c1ccc(-c2ccc(N(c3ccc4c(c3)oc3ccccc34)c3cc4nc(-c5ccccc5)oc4c4c3oc3ccccc34)cc2)cc1 ; c1ccc(-c2ccc(N(c3cc4oc5ccccc5c4c4oc(-c5ccccc5)nc34)c3cccc4c3oc3ccccc34)cc2)cc1 ; c1ccc(-c2ccc(N(c3ccc4oc5ccccc5c4c3)c3cccc4c3oc3ccc5nc(-c6ccccc6)oc5c34)cc2)cc1]

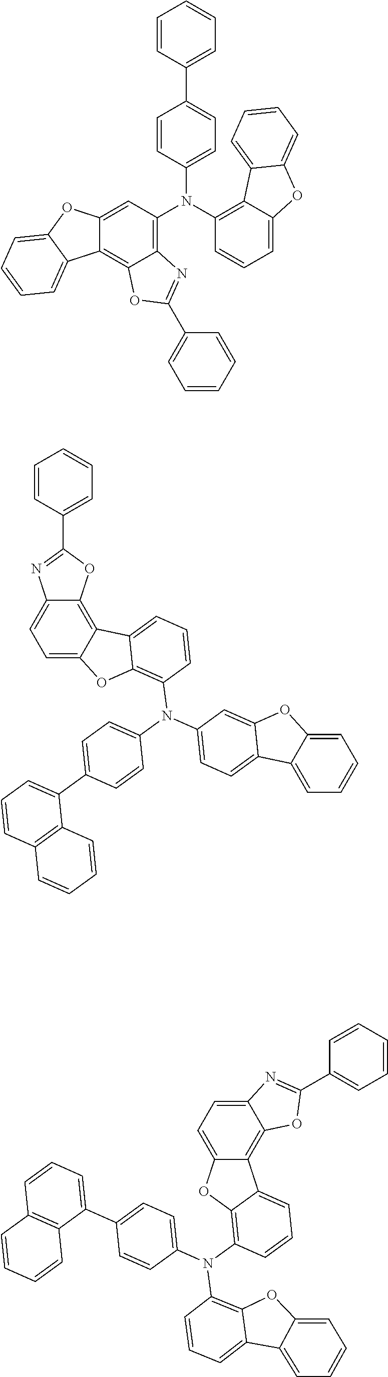 [SMILES: c1ccc(-c2ccc(N(c3cc4oc5ccccc5c4c4oc(-c5ccccc5)nc34)c3cccc4oc5ccccc5c34)cc2)cc1 ; c1ccc(-c2nc3ccc4oc5c(N(c6ccc(-c7cccc8ccccc78)cc6)c6ccc7c(c6)oc6ccccc67)cccc5c4c3o2)cc1 ; c1ccc(-c2nc3ccc4oc5c(N(c6ccc(-c7cccc8ccccc78)cc6)c6cccc7c6oc6ccccc67)cccc5c4c3o2)cc1]

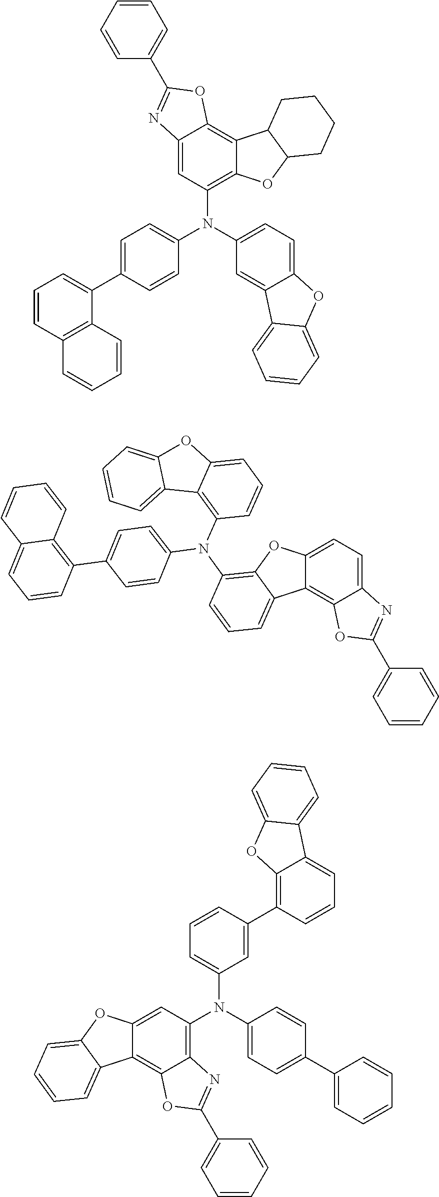 [SMILES: c1ccc(-c2nc3cc(N(c4ccc(-c5cccc6ccccc56)cc4)c4ccc5oc6ccccc6c5c4)c4c(c3o2)C2CCCCC2O4)cc1 ; c1ccc(-c2nc3ccc4oc5c(N(c6ccc(-c7cccc8ccccc78)cc6)c6cccc7oc8ccccc8c67)cccc5c4c3o2)cc1 ; c1ccc(-c2ccc(N(c3cccc(-c4cccc5c4oc4ccccc45)c3)c3cc4oc5ccccc5c4c4oc(-c5ccccc5)nc34)cc2)cc1]

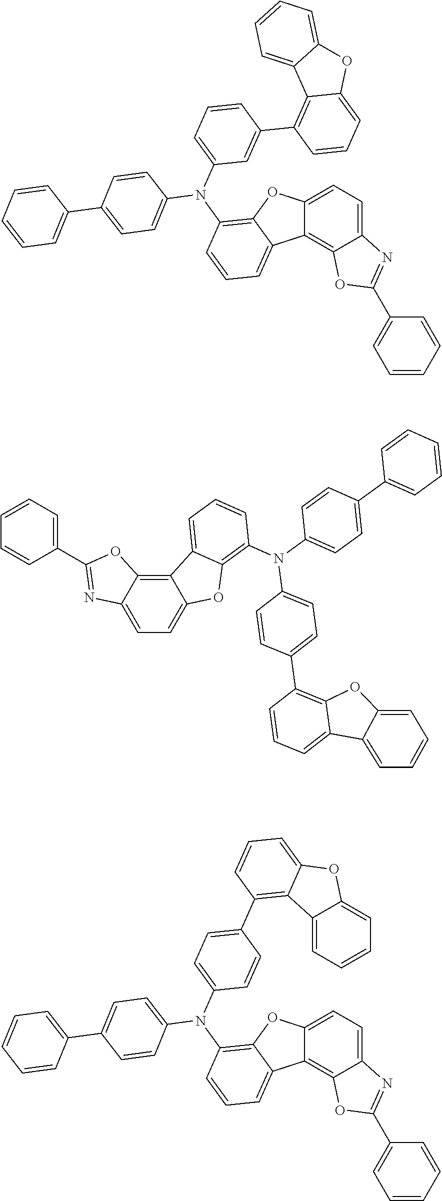 [SMILES: c1ccc(-c2ccc(N(c3cccc(-c4cccc5oc6ccccc6c45)c3)c3cccc4c3oc3ccc5nc(-c6ccccc6)oc5c34)cc2)cc1 ; c1ccc(-c2ccc(N(c3ccc(-c4cccc5c4oc4ccccc45)cc3)c3cccc4c3oc3ccc5nc(-c6ccccc6)oc5c34)cc2)cc1 ; c1ccc(-c2ccc(N(c3ccc(-c4cccc5oc6ccccc6c45)cc3)c3cccc4c3oc3ccc5nc(-c6ccccc6)oc5c34)cc2)cc1]

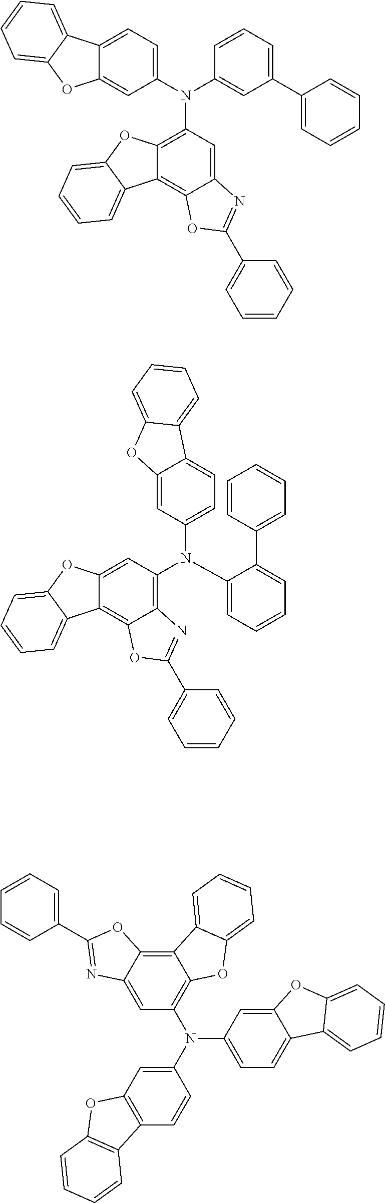 [SMILES: c1ccc(-c2cccc(N(c3ccc4c(c3)oc3ccccc34)c3cc4nc(-c5ccccc5)oc4c4c3oc3ccccc34)c2)cc1 ; c1ccc(-c2nc3c(N(c4ccc5c(c4)oc4ccccc45)c4ccccc4-c4ccccc4)cc4oc5ccccc5c4c3o2)cc1 ; c1ccc(-c2nc3cc(N(c4ccc5c(c4)oc4ccccc45)c4ccc5c(c4)oc4ccccc45)c4oc5ccccc5c4c3o2)cc1]

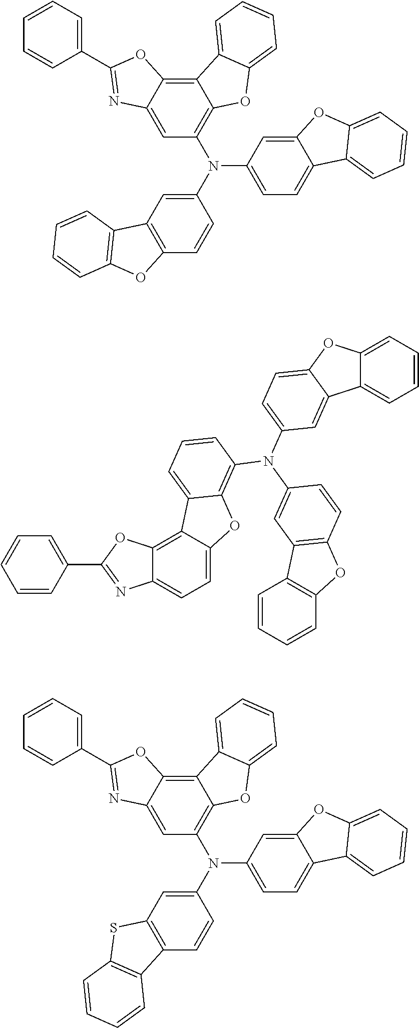 [SMILES: c1ccc(-c2nc3cc(N(c4ccc5c(c4)oc4ccccc45)c4ccc5oc6ccccc6c5c4)c4oc5ccccc5c4c3o2)cc1 ; c1ccc(-c2nc3ccc4oc5c(N(c6ccc7oc8ccccc8c7c6)c6ccc7oc8ccccc8c7c6)cccc5c4c3o2)cc1 ; c1ccc(-c2nc3cc(N(c4ccc5c(c4)oc4ccccc45)c4ccc5c(c4)sc4ccccc45)c4oc5ccccc5c4c3o2)cc1]

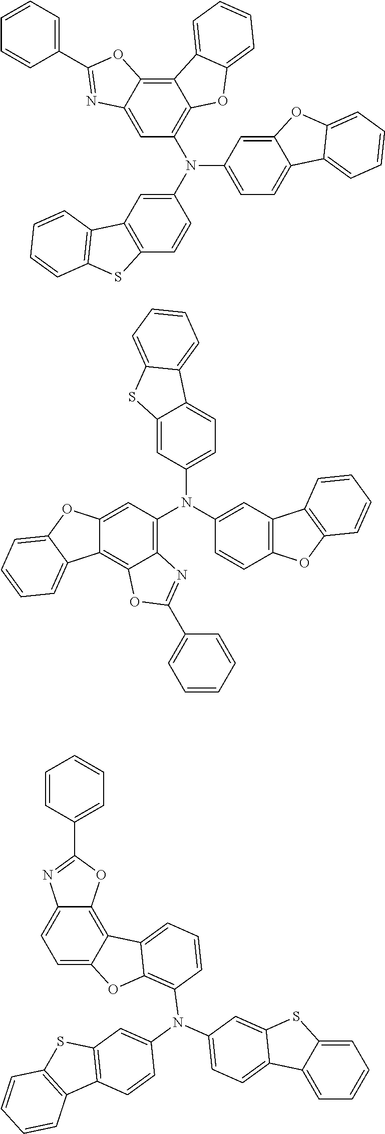

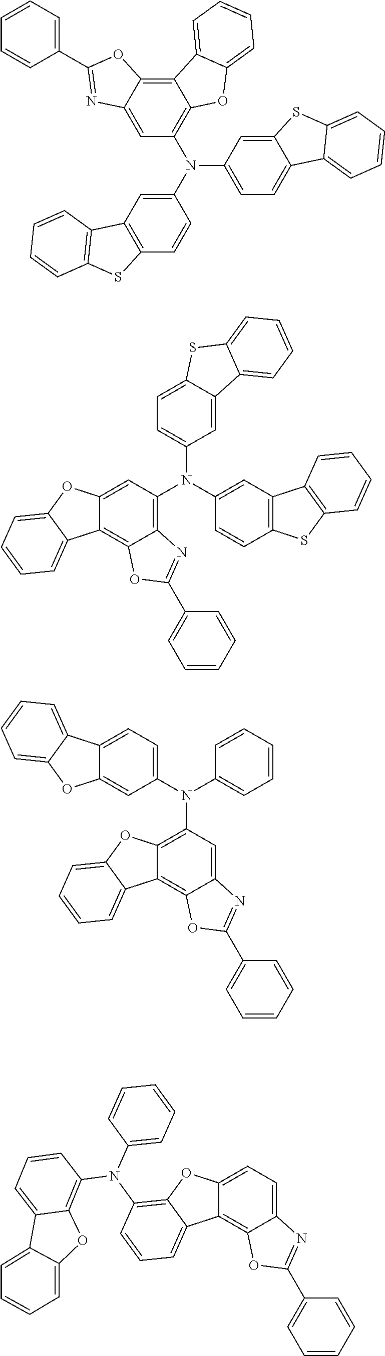 [SMILES: c1ccc(-c2nc3cc(N(c4ccc5c(c4)sc4ccccc45)c4ccc5sc6ccccc6c5c4)c4oc5ccccc5c4c3o2)cc1 ; c1ccc(-c2nc3c(N(c4ccc5sc6ccccc6c5c4)c4ccc5sc6ccccc6c5c4)cc4oc5ccccc5c4c3o2)cc1 ; c1ccc(-c2nc3cc(N(c4ccccc4)c4ccc5c(c4)oc4ccccc45)c4oc5ccccc5c4c3o2)cc1 ; c1ccc(-c2nc3ccc4oc5c(N(c6ccccc6)c6cccc7c6oc6ccccc67)cccc5c4c3o2)cc1]

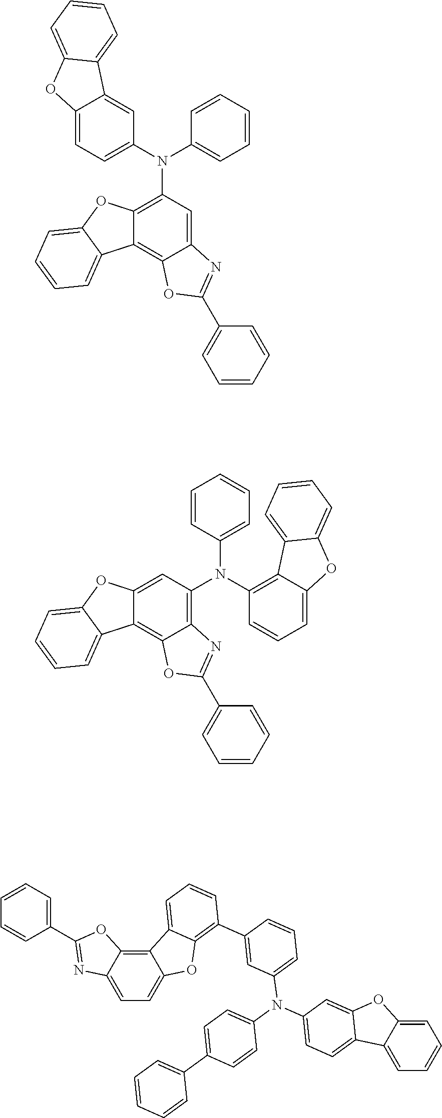 [SMILES: c1ccc(-c2nc3cc(N(c4ccccc4)c4ccc5oc6ccccc6c5c4)c4oc5ccccc5c4c3o2)cc1 ; c1ccc(-c2nc3c(N(c4ccccc4)c4cccc5oc6ccccc6c45)cc4oc5ccccc5c4c3o2)cc1 ; c1ccc(-c2ccc(N(c3cccc(-c4cccc5c4oc4ccc6nc(-c7ccccc7)oc6c45)c3)c3ccc4c(c3)oc3ccccc34)cc2)cc1]

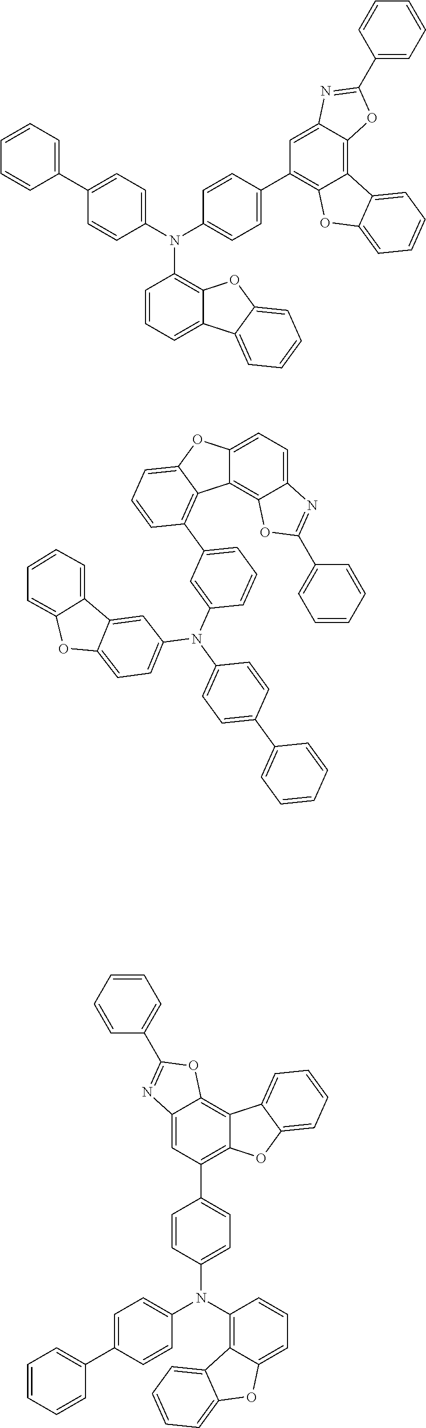 [SMILES: c1ccc(-c2ccc(N(c3ccc(-c4cc5nc(-c6ccccc6)oc5c5c4oc4ccccc45)cc3)c3cccc4c3oc3ccccc34)cc2)cc1 ; c1ccc(-c2ccc(N(c3cccc(-c4cccc5oc6ccc7nc(-c8ccccc8)oc7c6c45)c3)c3ccc4oc5ccccc5c4c3)cc2)cc1 ; c1ccc(-c2ccc(N(c3ccc(-c4cc5nc(-c6ccccc6)oc5c5c4oc4ccccc45)cc3)c3cccc4oc5ccccc5c34)cc2)cc1]

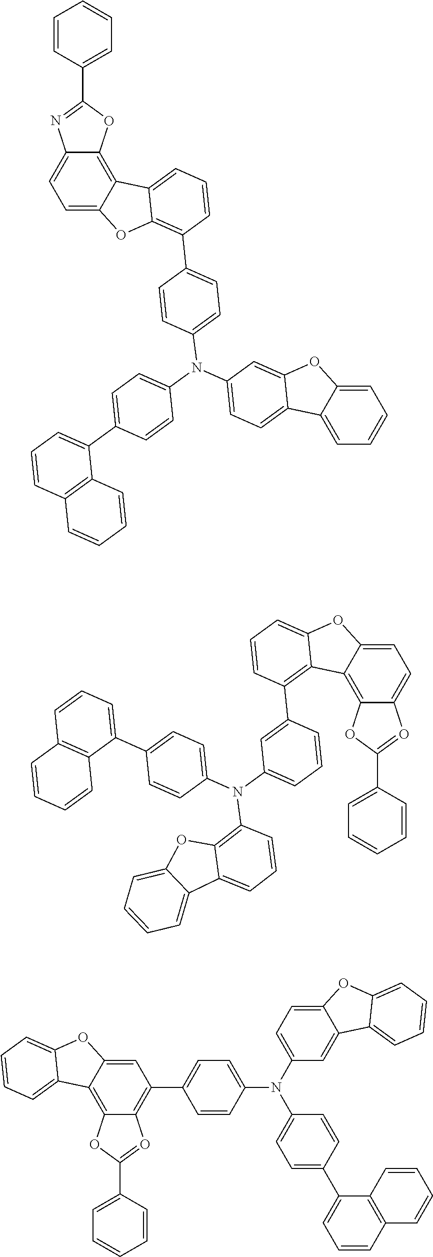

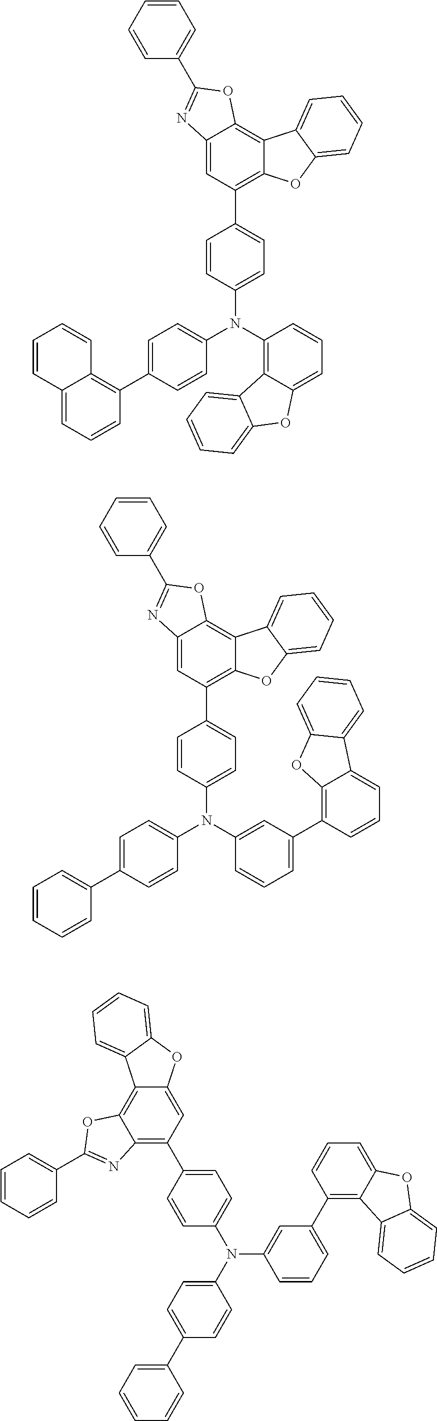 [SMILES: c1ccc(-c2nc3cc(-c4ccc(N(c5ccc(-c6cccc7ccccc67)cc5)c5cccc6oc7ccccc7c56)cc4)c4oc5ccccc5c4c3o2)cc1 ; c1ccc(-c2ccc(N(c3ccc(-c4cc5nc(-c6ccccc6)oc5c5c4oc4ccccc45)cc3)c3cccc(-c4cccc5c4oc4ccccc45)c3)cc2)cc1 ; c1ccc(-c2ccc(N(c3ccc(-c4cc5oc6ccccc6c5c5oc(-c6ccccc6)nc45)cc3)c3cccc(-c4cccc5oc6ccccc6c45)c3)cc2)cc1]

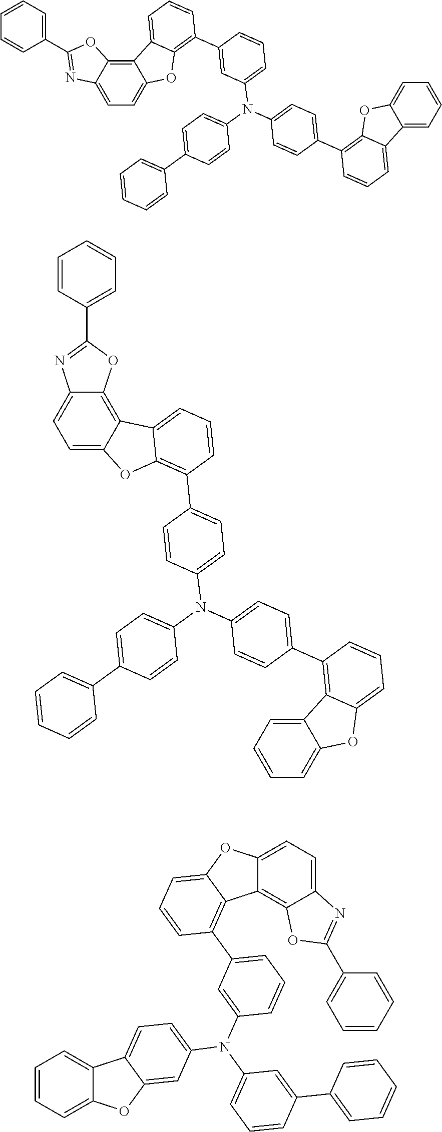 [SMILES: c1ccc(-c2ccc(N(c3ccc(-c4cccc5c4oc4ccccc45)cc3)c3cccc(-c4cccc5c4oc4ccc6nc(-c7ccccc7)oc6c45)c3)cc2)cc1 ; c1ccc(-c2ccc(N(c3ccc(-c4cccc5c4oc4ccc6nc(-c7ccccc7)oc6c45)cc3)c3ccc(-c4cccc5oc6ccccc6c45)cc3)cc2)cc1 ; c1ccc(-c2cccc(N(c3cccc(-c4cccc5oc6ccc7nc(-c8ccccc8)oc7c6c45)c3)c3ccc4c(c3)oc3ccccc34)c2)cc1]

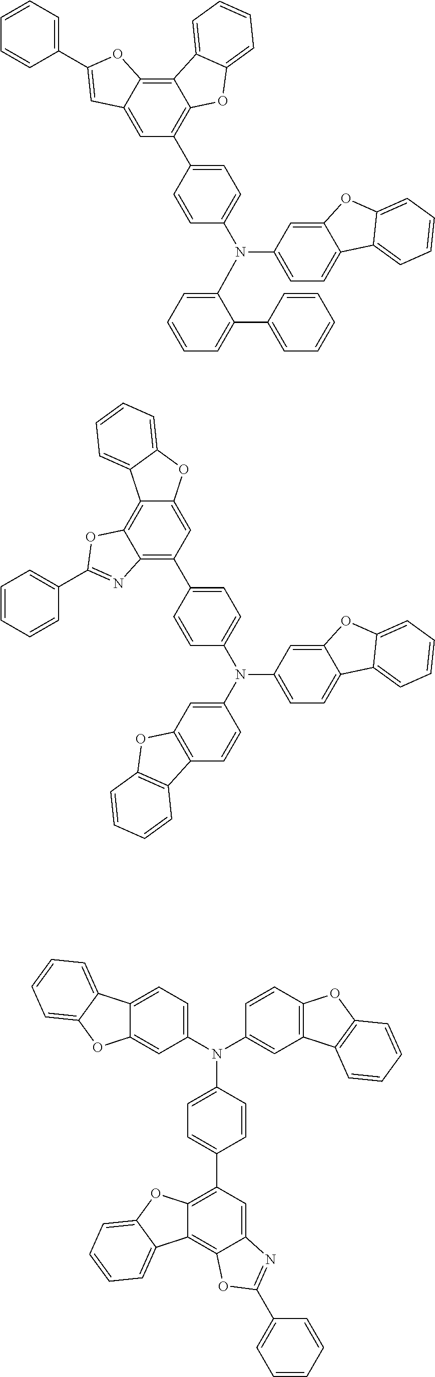 [SMILES: c1ccc(-c2cc3cc(-c4ccc(N(c5ccc6c(c5)oc5ccccc56)c5ccccc5-c5ccccc5)cc4)c4oc5ccccc5c4c3o2)cc1 ; c1ccc(-c2nc3c(-c4ccc(N(c5ccc6c(c5)oc5ccccc56)c5ccc6c(c5)oc5ccccc56)cc4)cc4oc5ccccc5c4c3o2)cc1 ; c1ccc(-c2nc3cc(-c4ccc(N(c5ccc6c(c5)oc5ccccc56)c5ccc6oc7ccccc7c6c5)cc4)c4oc5ccccc5c4c3o2)cc1]

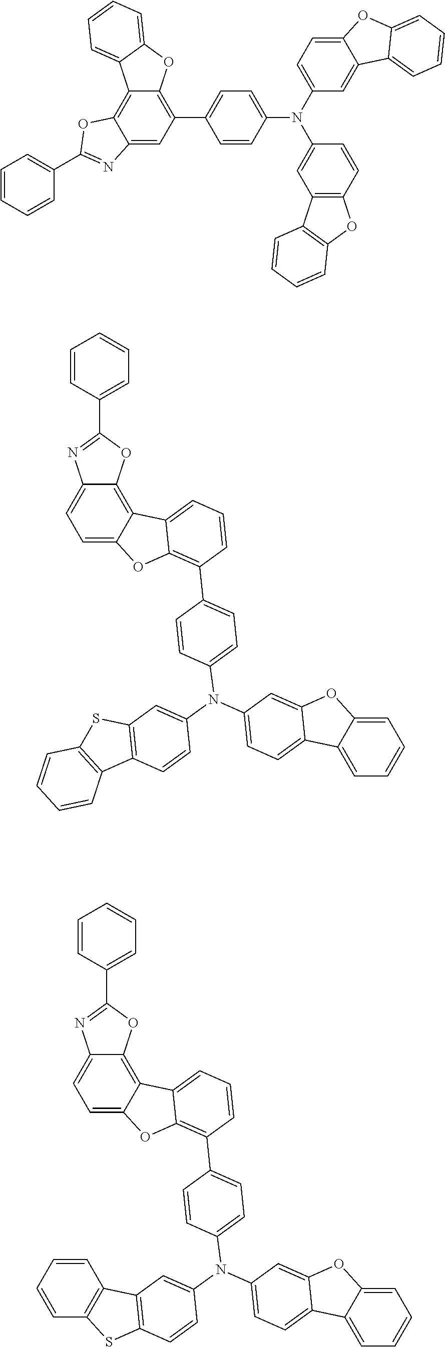 [SMILES: c1ccc(-c2nc3cc(-c4ccc(N(c5ccc6oc7ccccc7c6c5)c5ccc6oc7ccccc7c6c5)cc4)c4oc5ccccc5c4c3o2)cc1 ; c1ccc(-c2nc3ccc4oc5c(-c6ccc(N(c7ccc8c(c7)oc7ccccc78)c7ccc8c(c7)sc7ccccc78)cc6)cccc5c4c3o2)cc1 ; c1ccc(-c2nc3ccc4oc5c(-c6ccc(N(c7ccc8c(c7)oc7ccccc78)c7ccc8sc9ccccc9c8c7)cc6)cccc5c4c3o2)cc1]

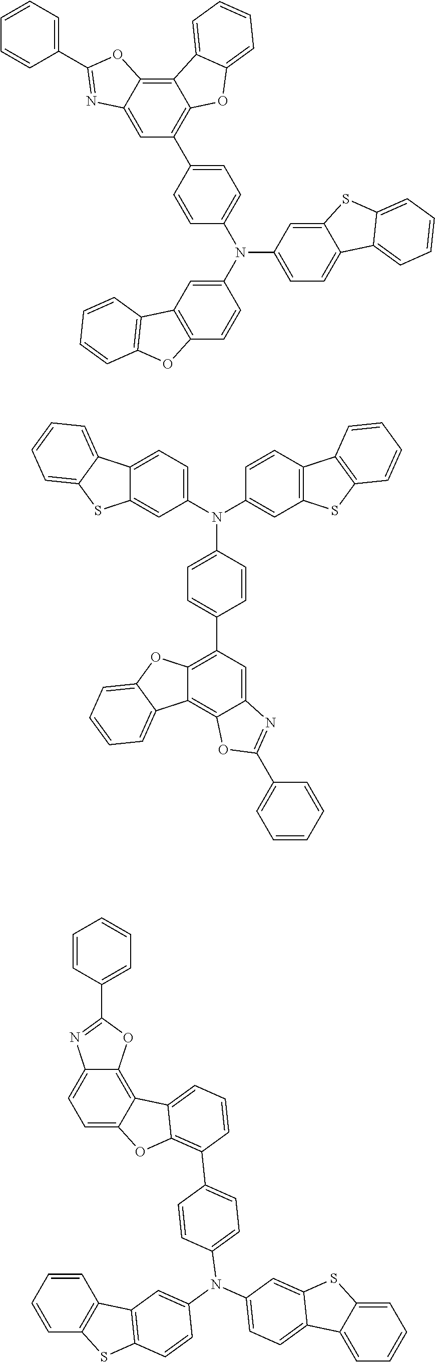 [SMILES: c1ccc(-c2nc3cc(-c4ccc(N(c5ccc6c(c5)sc5ccccc56)c5ccc6oc7ccccc7c6c5)cc4)c4oc5ccccc5c4c3o2)cc1 ; c1ccc(-c2nc3cc(-c4ccc(N(c5ccc6c(c5)sc5ccccc56)c5ccc6c(c5)sc5ccccc56)cc4)c4oc5ccccc5c4c3o2)cc1 ; c1ccc(-c2nc3ccc4oc5c(-c6ccc(N(c7ccc8c(c7)sc7ccccc78)c7ccc8sc9ccccc9c8c7)cc6)cccc5c4c3o2)cc1]

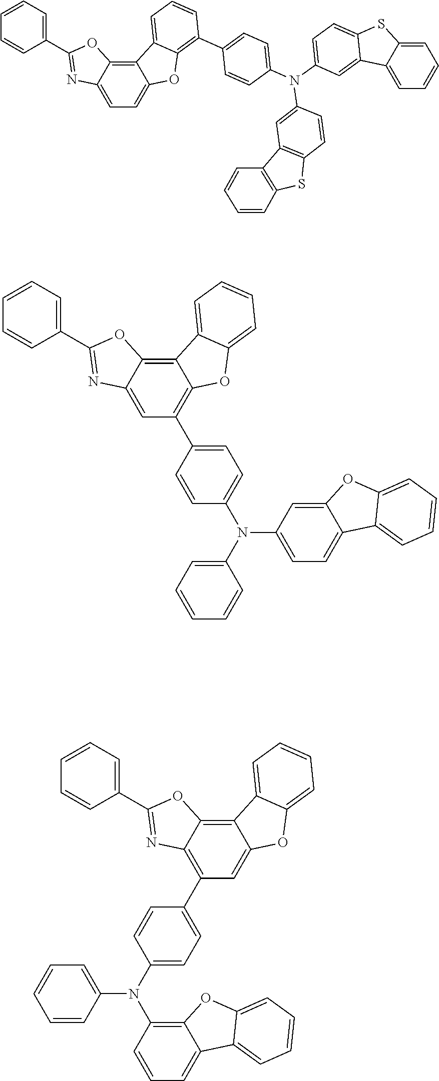 [SMILES: c1ccc(-c2nc3ccc4oc5c(-c6ccc(N(c7ccc8sc9ccccc9c8c7)c7ccc8sc9ccccc9c8c7)cc6)cccc5c4c3o2)cc1 ; c1ccc(-c2nc3cc(-c4ccc(N(c5ccccc5)c5ccc6c(c5)oc5ccccc56)cc4)c4oc5ccccc5c4c3o2)cc1 ; c1ccc(-c2nc3c(-c4ccc(N(c5ccccc5)c5cccc6c5oc5ccccc56)cc4)cc4oc5ccccc5c4c3o2)cc1]

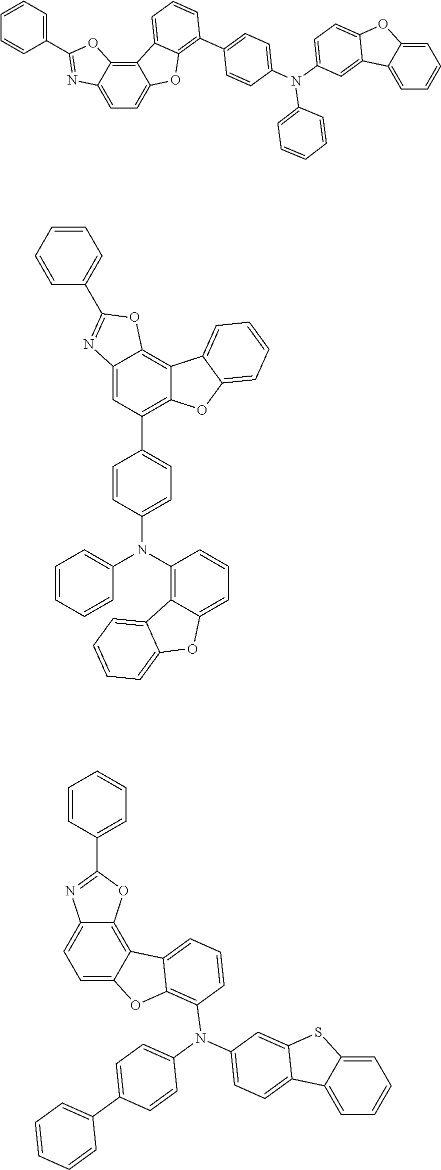 [SMILES: c1ccc(-c2nc3ccc4oc5c(-c6ccc(N(c7ccccc7)c7ccc8oc9ccccc9c8c7)cc6)cccc5c4c3o2)cc1 ; c1ccc(-c2nc3cc(-c4ccc(N(c5ccccc5)c5cccc6oc7ccccc7c56)cc4)c4oc5ccccc5c4c3o2)cc1 ; c1ccc(-c2ccc(N(c3ccc4c(c3)sc3ccccc34)c3cccc4c3oc3ccc5nc(-c6ccccc6)oc5c34)cc2)cc1]

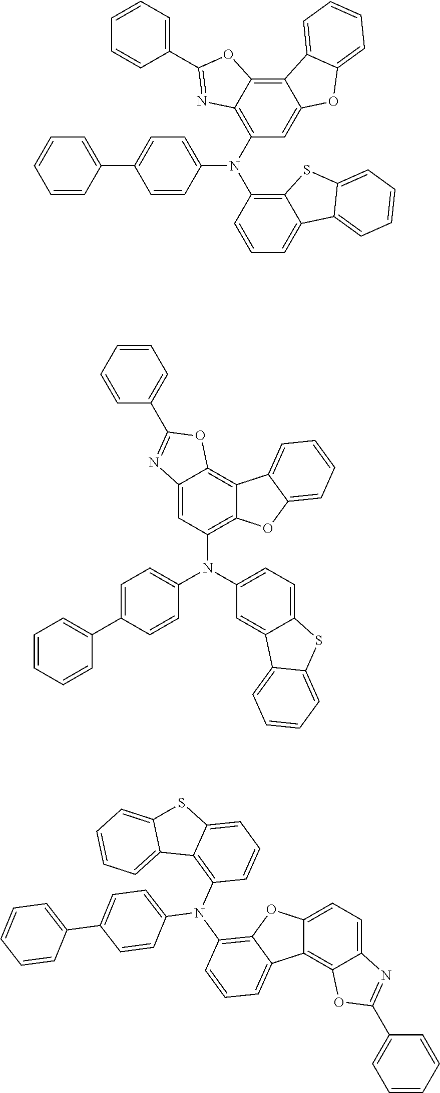 [SMILES: c1ccc(-c2ccc(N(c3cc4oc5ccccc5c4c4oc(-c5ccccc5)nc34)c3cccc4c3sc3ccccc34)cc2)cc1 ; c1ccc(-c2ccc(N(c3ccc4sc5ccccc5c4c3)c3cc4nc(-c5ccccc5)oc4c4c3oc3ccccc34)cc2)cc1 ; c1ccc(-c2ccc(N(c3cccc4c3oc3ccc5nc(-c6ccccc6)oc5c34)c3cccc4sc5ccccc5c34)cc2)cc1]

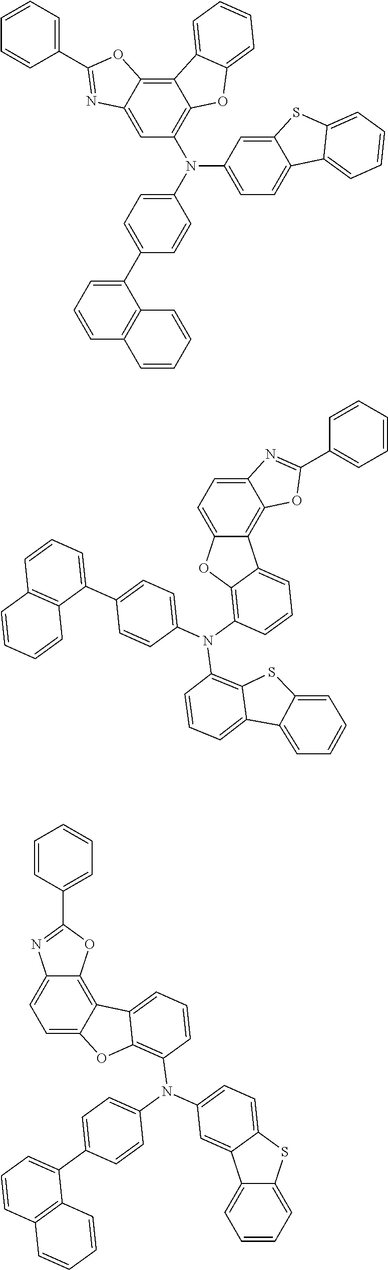 [SMILES: c1ccc(-c2nc3cc(N(c4ccc(-c5cccc6ccccc56)cc4)c4ccc5c(c4)sc4ccccc45)c4oc5ccccc5c4c3o2)cc1 ; c1ccc(-c2nc3ccc4oc5c(N(c6ccc(-c7cccc8ccccc78)cc6)c6cccc7c6sc6ccccc67)cccc5c4c3o2)cc1 ; c1ccc(-c2nc3ccc4oc5c(N(c6ccc(-c7cccc8ccccc78)cc6)c6ccc7sc8ccccc8c7c6)cccc5c4c3o2)cc1]

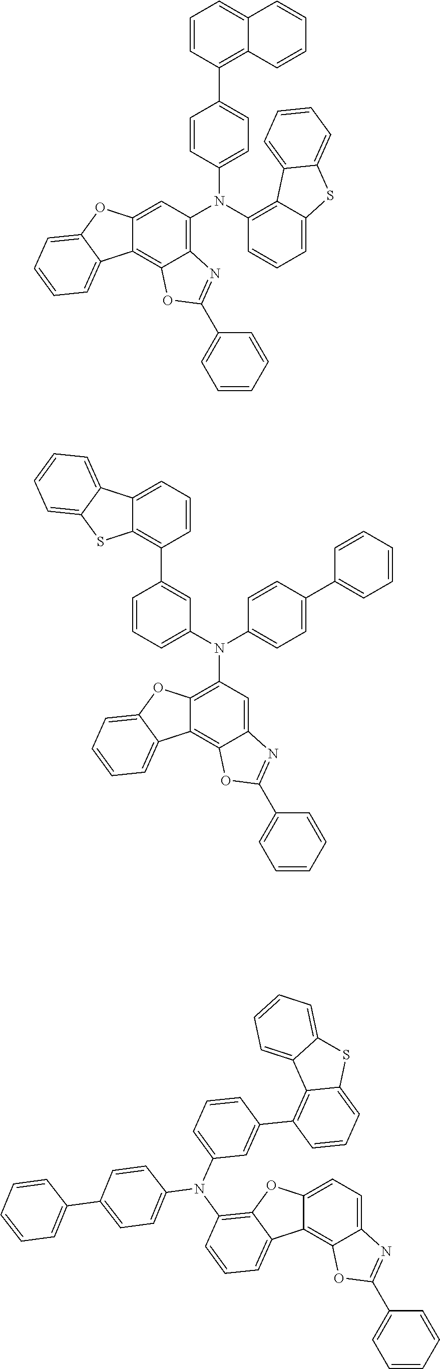 [SMILES: c1ccc(-c2nc3c(N(c4ccc(-c5cccc6ccccc56)cc4)c4cccc5sc6ccccc6c45)cc4oc5ccccc5c4c3o2)cc1 ; c1ccc(-c2ccc(N(c3cccc(-c4cccc5c4sc4ccccc45)c3)c3cc4nc(-c5ccccc5)oc4c4c3oc3ccccc34)cc2)cc1 ; c1ccc(-c2ccc(N(c3cccc(-c4cccc5sc6ccccc6c45)c3)c3cccc4c3oc3ccc5nc(-c6ccccc6)oc5c34)cc2)cc1]

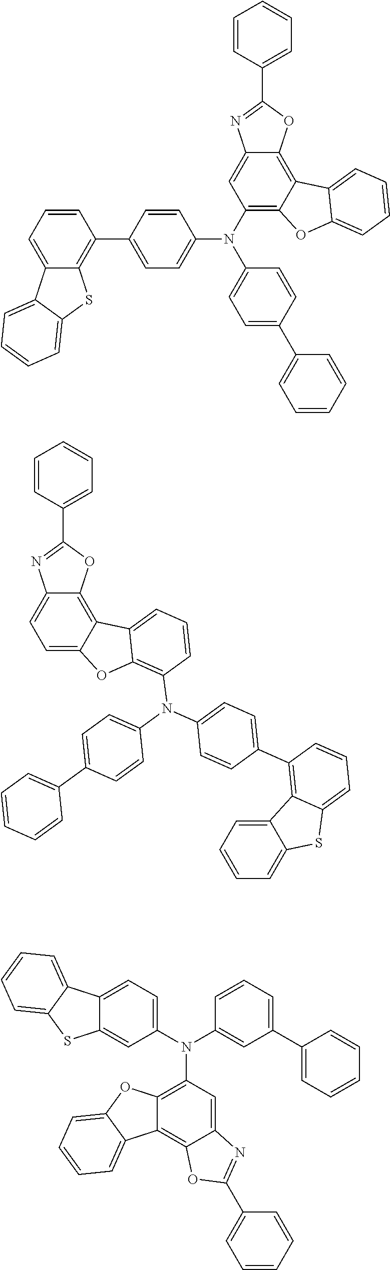 [SMILES: c1ccc(-c2ccc(N(c3ccc(-c4cccc5c4sc4ccccc45)cc3)c3cc4nc(-c5ccccc5)oc4c4c3oc3ccccc34)cc2)cc1 ; c1ccc(-c2ccc(N(c3ccc(-c4cccc5sc6ccccc6c45)cc3)c3cccc4c3oc3ccc5nc(-c6ccccc6)oc5c34)cc2)cc1 ; c1ccc(-c2cccc(N(c3ccc4c(c3)sc3ccccc34)c3cc4nc(-c5ccccc5)oc4c4c3oc3ccccc34)c2)cc1]

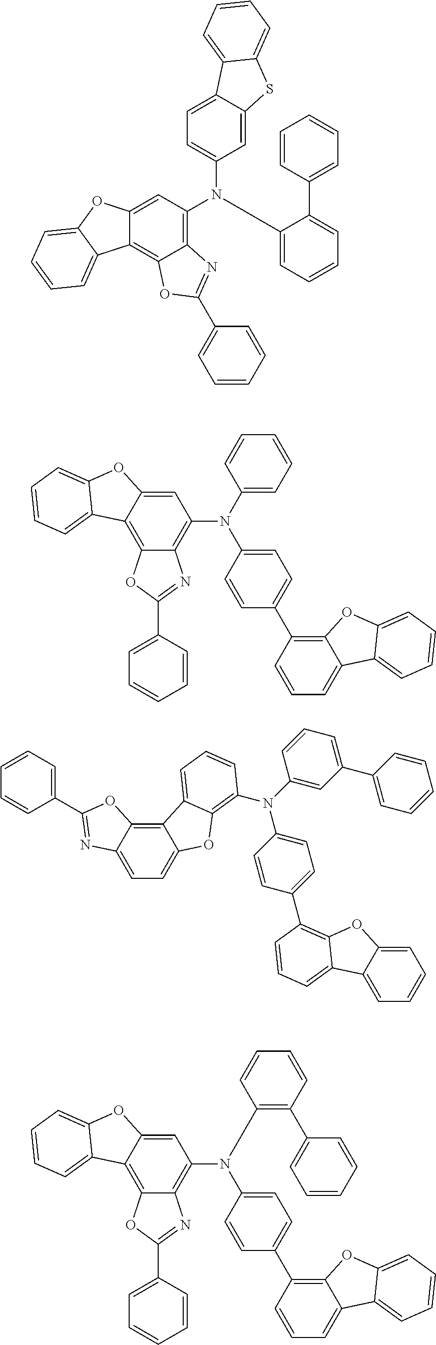

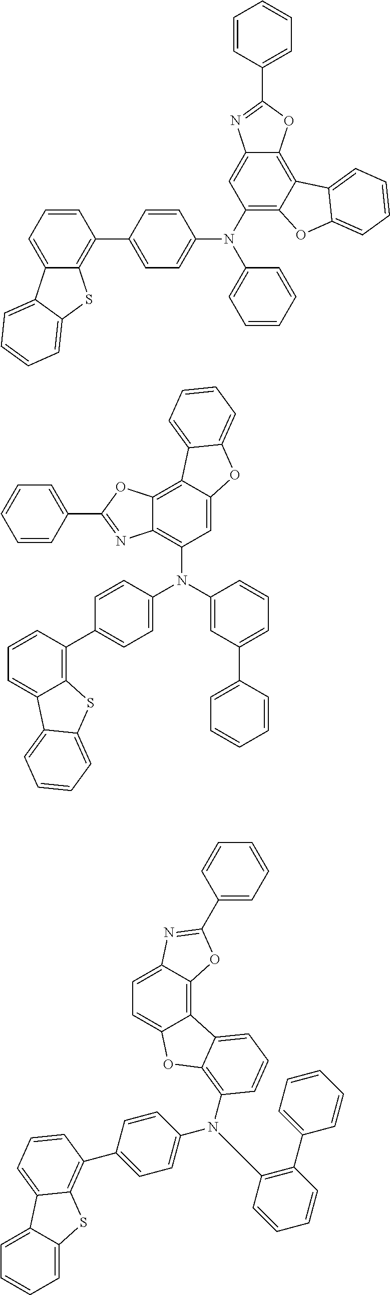 [SMILES: c1ccc(-c2nc3cc(N(c4ccccc4)c4ccc(-c5cccc6c5sc5ccccc56)cc4)c4oc5ccccc5c4c3o2)cc1 ; c1ccc(-c2cccc(N(c3ccc(-c4cccc5c4sc4ccccc45)cc3)c3cc4oc5ccccc5c4c4oc(-c5ccccc5)nc34)c2)cc1 ; c1ccc(-c2nc3ccc4oc5c(N(c6ccc(-c7cccc8c7sc7ccccc78)cc6)c6ccccc6-c6ccccc6)cccc5c4c3o2)cc1]

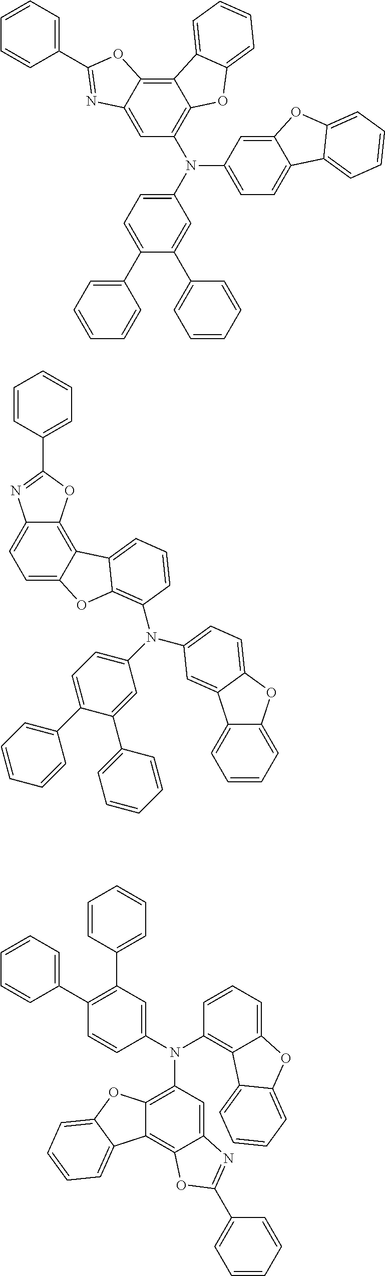 [SMILES: c1ccc(-c2nc3cc(N(c4ccc(-c5ccccc5)c(-c5ccccc5)c4)c4ccc5c(c4)oc4ccccc45)c4oc5ccccc5c4c3o2)cc1 ; c1ccc(-c2nc3ccc4oc5c(N(c6ccc(-c7ccccc7)c(-c7ccccc7)c6)c6ccc7oc8ccccc8c7c6)cccc5c4c3o2)cc1 ; c1ccc(-c2nc3cc(N(c4ccc(-c5ccccc5)c(-c5ccccc5)c4)c4cccc5oc6ccccc6c45)c4oc5ccccc5c4c3o2)cc1]

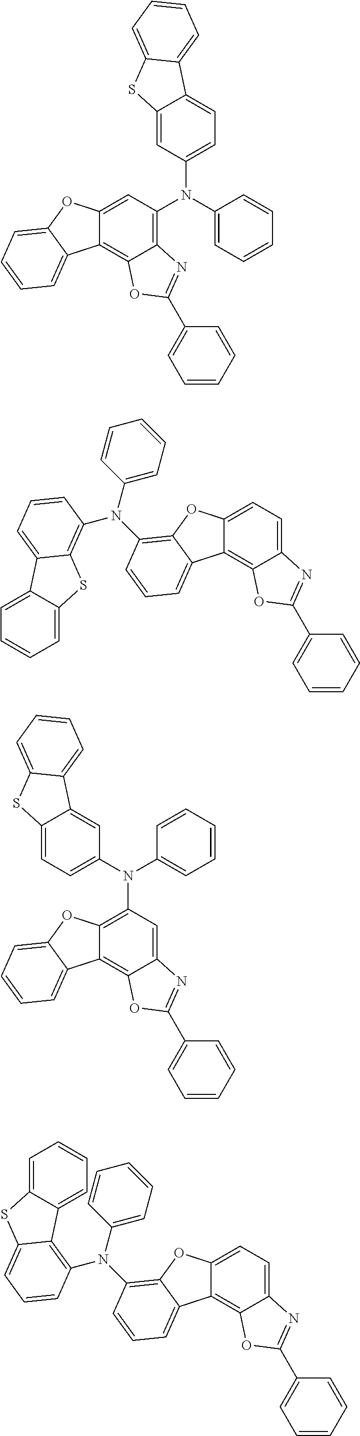 [SMILES: c1ccc(-c2nc3c(N(c4ccccc4)c4ccc5c(c4)sc4ccccc45)cc4oc5ccccc5c4c3o2)cc1 ; c1ccc(-c2nc3ccc4oc5c(N(c6ccccc6)c6cccc7c6sc6ccccc67)cccc5c4c3o2)cc1 ; c1ccc(-c2nc3cc(N(c4ccccc4)c4ccc5sc6ccccc6c5c4)c4oc5ccccc5c4c3o2)cc1 ; c1ccc(-c2nc3ccc4oc5c(N(c6ccccc6)c6cccc7sc8ccccc8c67)cccc5c4c3o2)cc1]

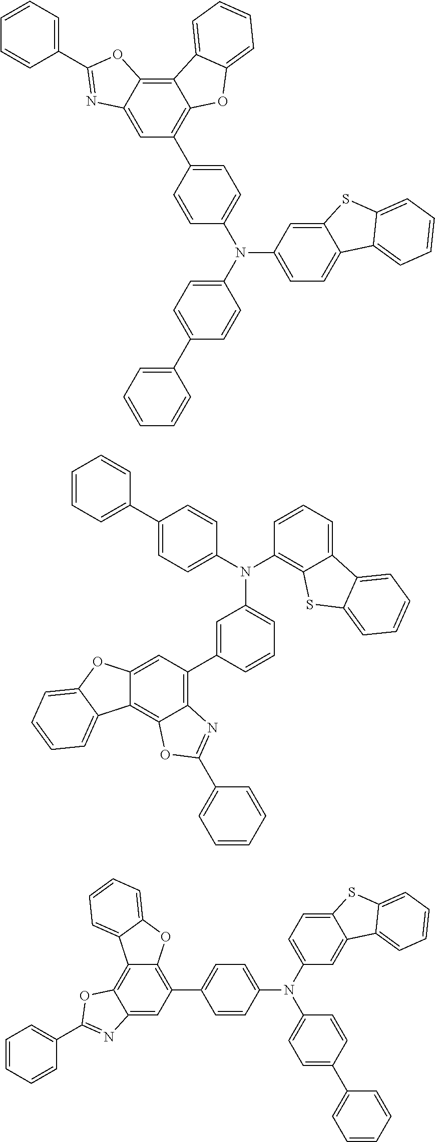 [SMILES: c1ccc(-c2ccc(N(c3ccc(-c4cc5nc(-c6ccccc6)oc5c5c4oc4ccccc45)cc3)c3ccc4c(c3)sc3ccccc34)cc2)cc1 ; c1ccc(-c2ccc(N(c3cccc(-c4cc5oc6ccccc6c5c5oc(-c6ccccc6)nc45)c3)c3cccc4c3sc3ccccc34)cc2)cc1 ; c1ccc(-c2ccc(N(c3ccc(-c4cc5nc(-c6ccccc6)oc5c5c4oc4ccccc45)cc3)c3ccc4sc5ccccc5c4c3)cc2)cc1]

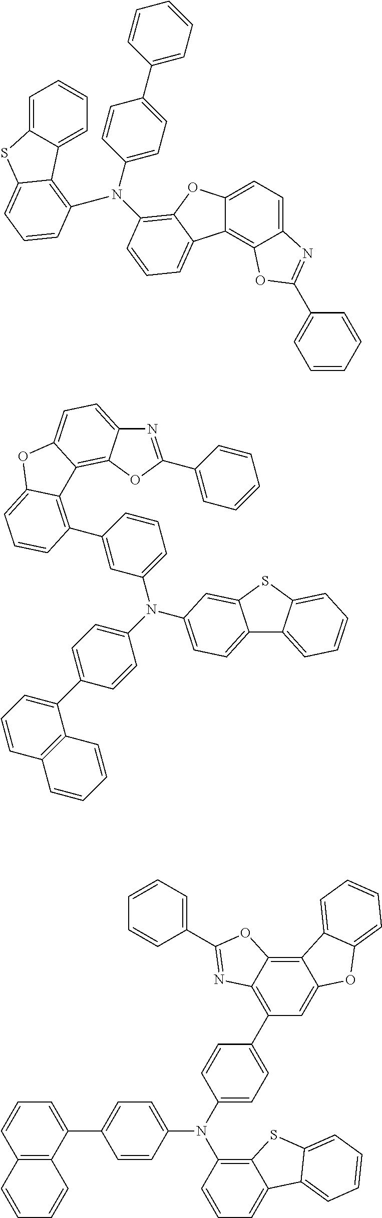 [SMILES: c1ccc(-c2ccc(N(c3cccc4c3oc3ccc5nc(-c6ccccc6)oc5c34)c3cccc4sc5ccccc5c34)cc2)cc1 ; c1ccc(-c2nc3ccc4oc5cccc(-c6cccc(N(c7ccc(-c8cccc9ccccc89)cc7)c7ccc8c(c7)sc7ccccc78)c6)c5c4c3o2)cc1 ; c1ccc(-c2nc3c(-c4ccc(N(c5ccc(-c6cccc7ccccc67)cc5)c5cccc6c5sc5ccccc56)cc4)cc4oc5ccccc5c4c3o2)cc1]

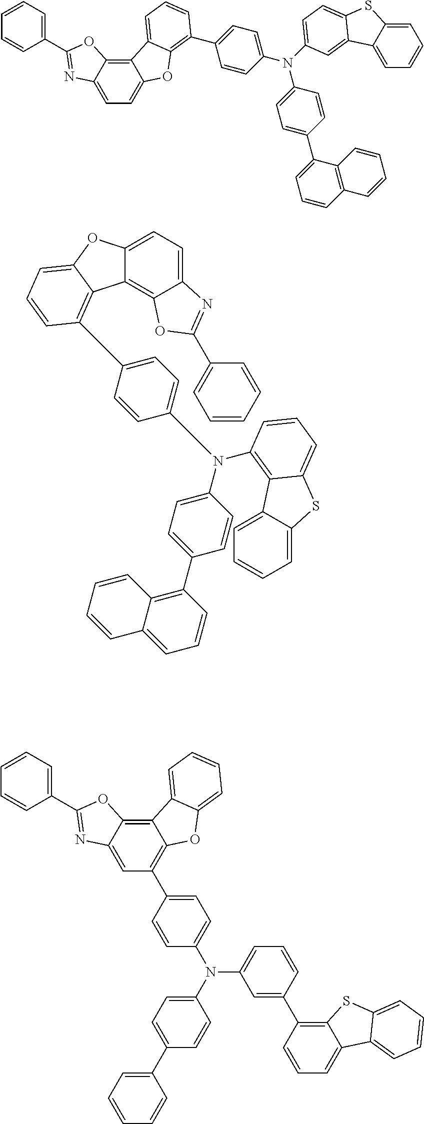 [SMILES: c1ccc(-c2nc3ccc4oc5c(-c6ccc(N(c7ccc(-c8cccc9ccccc89)cc7)c7ccc8sc9ccccc9c8c7)cc6)cccc5c4c3o2)cc1 ; c1ccc(-c2nc3ccc4oc5cccc(-c6ccc(N(c7ccc(-c8cccc9ccccc89)cc7)c7cccc8sc9ccccc9c78)cc6)c5c4c3o2)cc1 ; c1ccc(-c2ccc(N(c3ccc(-c4cc5nc(-c6ccccc6)oc5c5c4oc4ccccc45)cc3)c3cccc(-c4cccc5c4sc4ccccc45)c3)cc2)cc1]

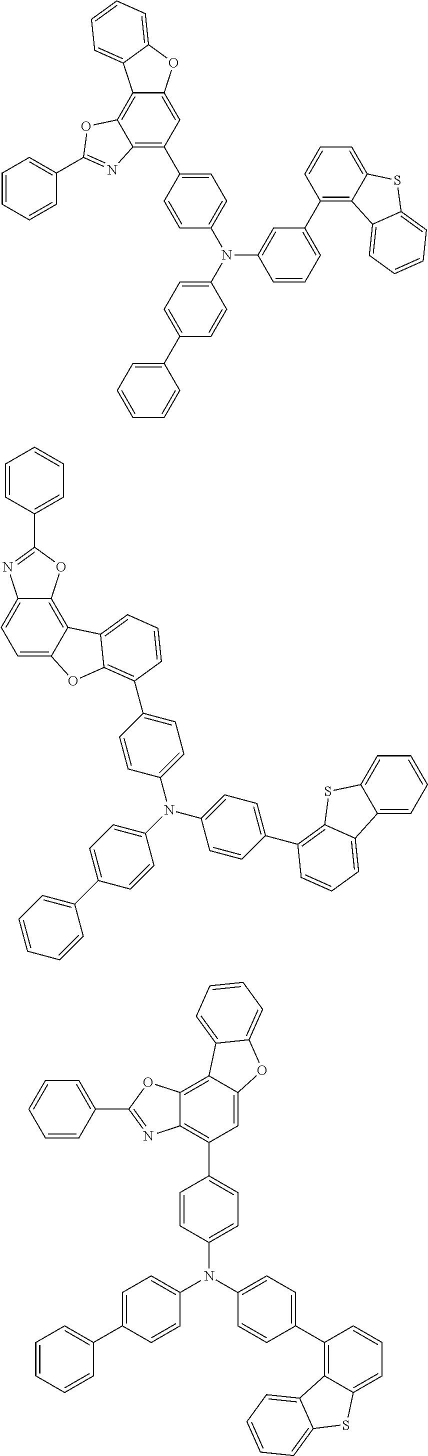 [SMILES: c1ccc(-c2ccc(N(c3ccc(-c4cc5oc6ccccc6c5c5oc(-c6ccccc6)nc45)cc3)c3cccc(-c4cccc5sc6ccccc6c45)c3)cc2)cc1 ; c1ccc(-c2ccc(N(c3ccc(-c4cccc5c4oc4ccc6nc(-c7ccccc7)oc6c45)cc3)c3ccc(-c4cccc5c4sc4ccccc45)cc3)cc2)cc1 ; c1ccc(-c2ccc(N(c3ccc(-c4cc5oc6ccccc6c5c5oc(-c6ccccc6)nc45)cc3)c3ccc(-c4cccc5sc6ccccc6c45)cc3)cc2)cc1]

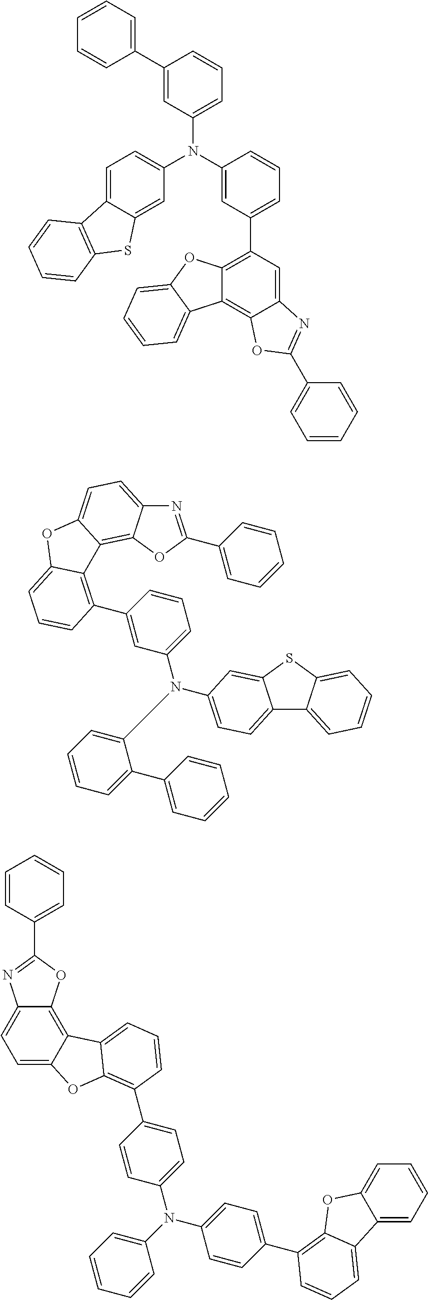 [SMILES: c1ccc(-c2cccc(N(c3cccc(-c4cc5nc(-c6ccccc6)oc5c5c4oc4ccccc45)c3)c3ccc4c(c3)sc3ccccc34)c2)cc1 ; c1ccc(-c2nc3ccc4oc5cccc(-c6cccc(N(c7ccc8c(c7)sc7ccccc78)c7ccccc7-c7ccccc7)c6)c5c4c3o2)cc1 ; c1ccc(-c2nc3ccc4oc5c(-c6ccc(N(c7ccccc7)c7ccc(-c8cccc9c8oc8ccccc89)cc7)cc6)cccc5c4c3o2)cc1]

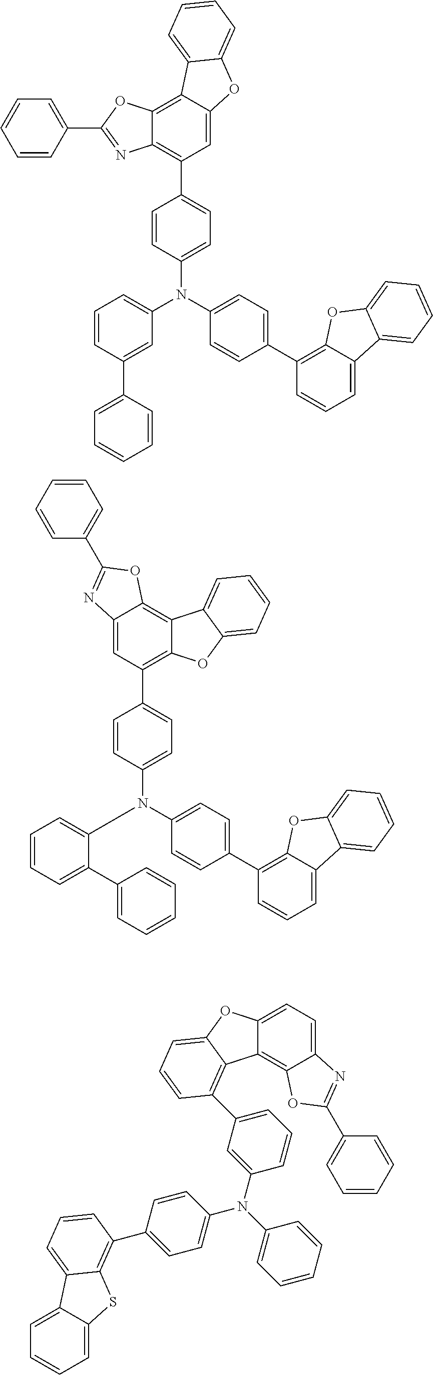

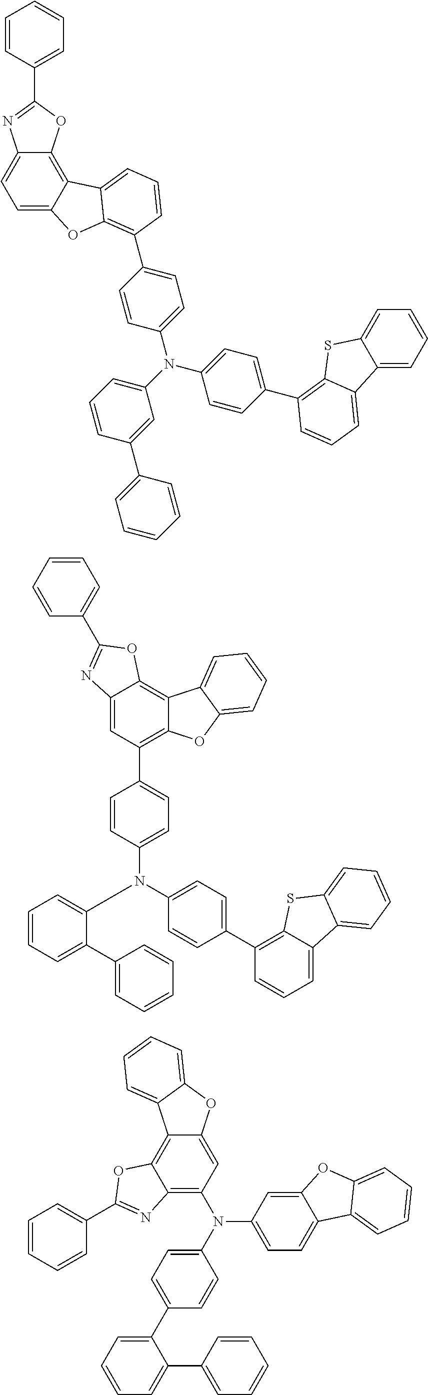

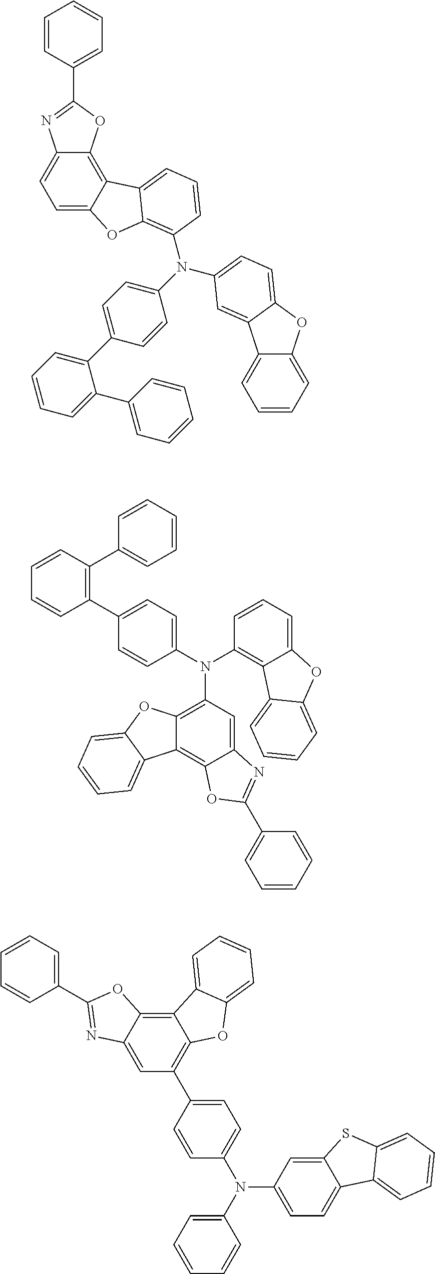 [SMILES: c1ccc(-c2nc3ccc4oc5c(N(c6ccc(-c7ccccc7-c7ccccc7)cc6)c6ccc7oc8ccccc8c7c6)cccc5c4c3o2)cc1 ; c1ccc(-c2nc3cc(N(c4ccc(-c5ccccc5-c5ccccc5)cc4)c4cccc5oc6ccccc6c45)c4oc5ccccc5c4c3o2)cc1 ; c1ccc(-c2nc3cc(-c4ccc(N(c5ccccc5)c5ccc6c(c5)sc5ccccc56)cc4)c4oc5ccccc5c4c3o2)cc1]

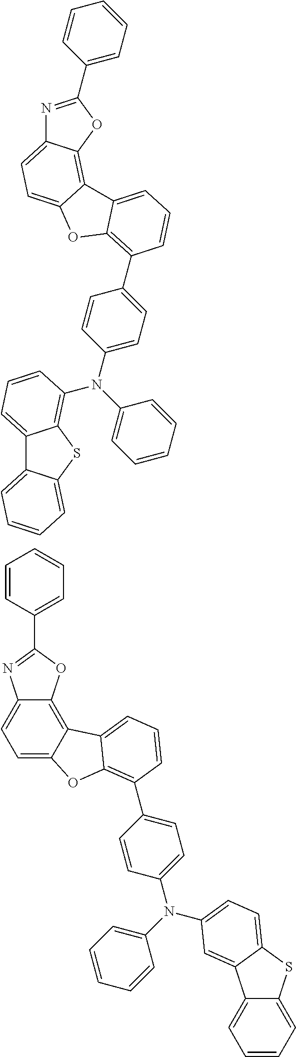

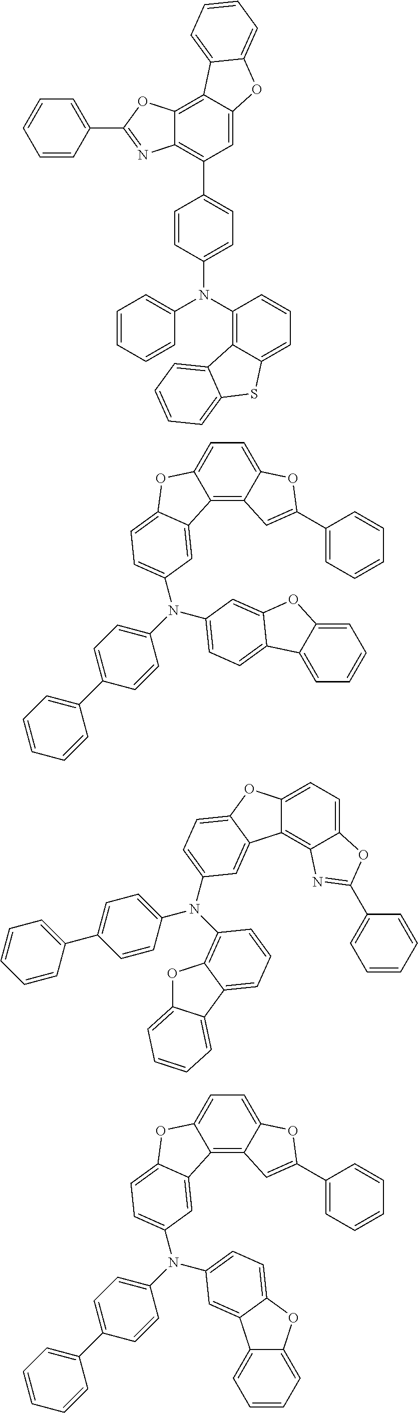

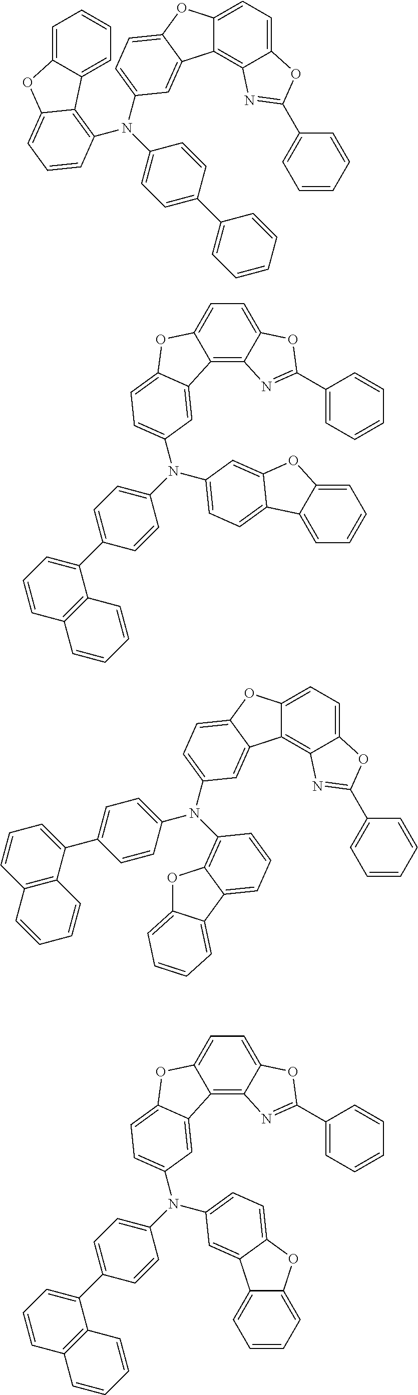

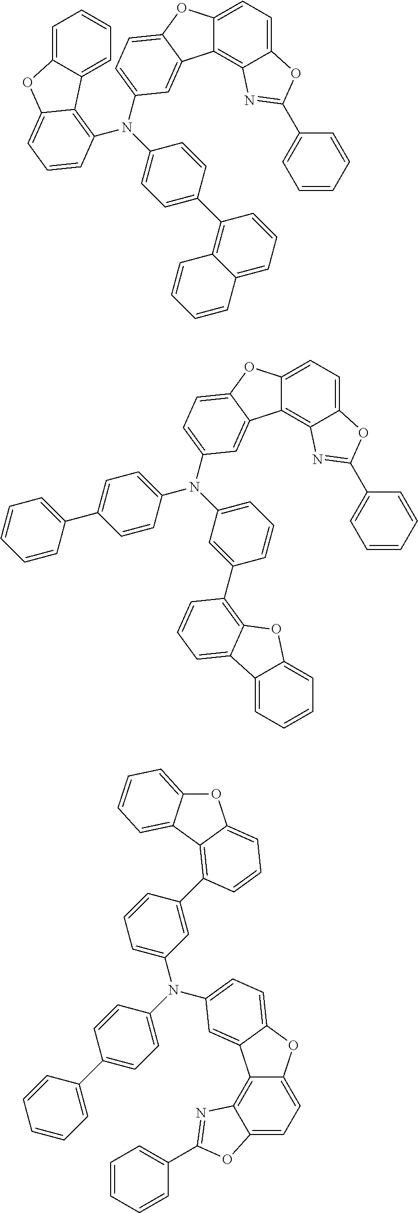 [SMILES: c1ccc(-c2nc3c(ccc4oc5ccc(N(c6ccc(-c7cccc8ccccc78)cc6)c6cccc7oc8ccccc8c67)cc5c43)o2)cc1 ; c1ccc(-c2ccc(N(c3cccc(-c4cccc5c4oc4ccccc45)c3)c3ccc4oc5ccc6oc(-c7ccccc7)nc6c5c4c3)cc2)cc1 ; c1ccc(-c2ccc(N(c3cccc(-c4cccc5oc6ccccc6c45)c3)c3ccc4oc5ccc6oc(-c7ccccc7)nc6c5c4c3)cc2)cc1]

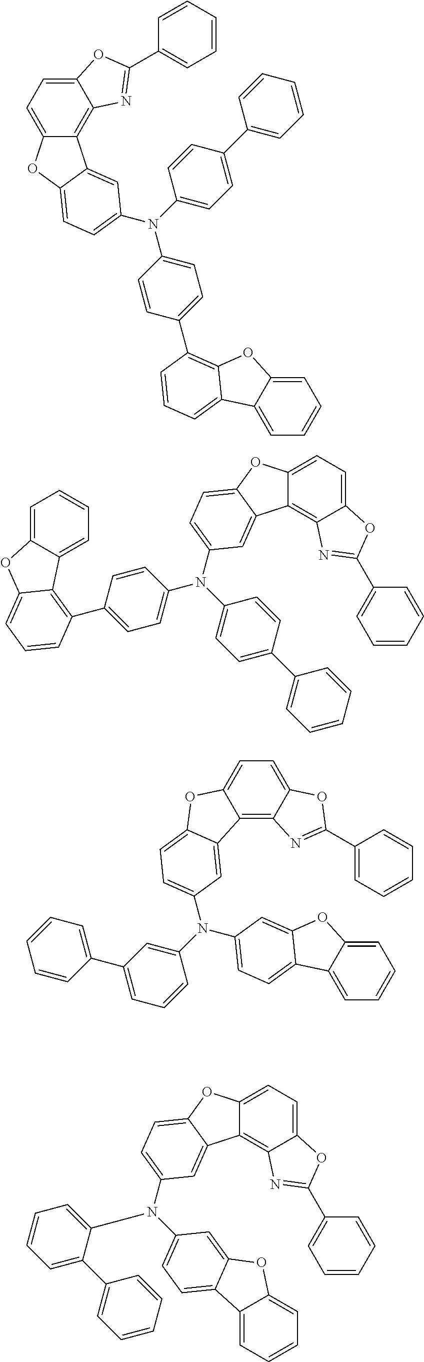

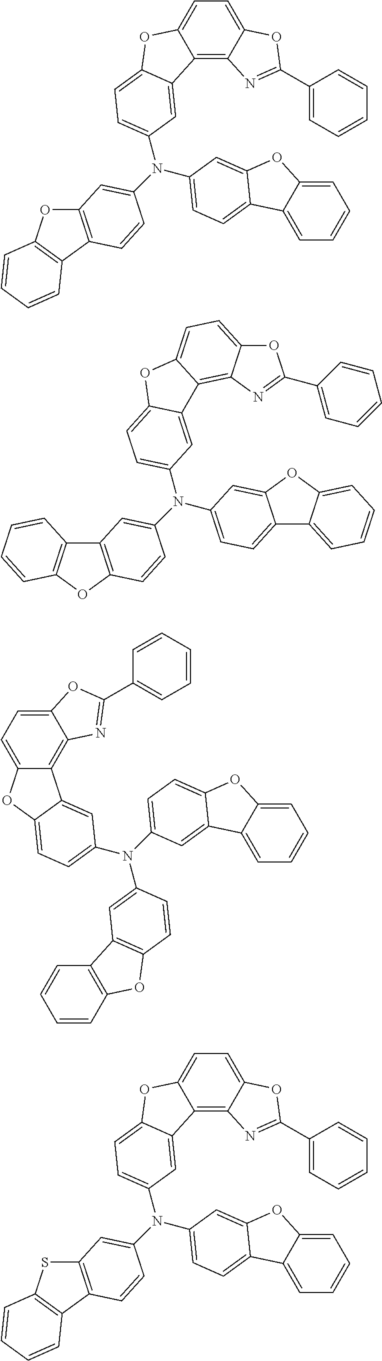 [SMILES: c1ccc(-c2nc3c(ccc4oc5ccc(N(c6ccc7c(c6)oc6ccccc67)c6ccc7c(c6)oc6ccccc67)cc5c43)o2)cc1 ; c1ccc(-c2nc3c(ccc4oc5ccc(N(c6ccc7c(c6)oc6ccccc67)c6ccc7oc8ccccc8c7c6)cc5c43)o2)cc1 ; c1ccc(-c2nc3c(ccc4oc5ccc(N(c6ccc7oc8ccccc8c7c6)c6ccc7oc8ccccc8c7c6)cc5c43)o2)cc1 ; c1ccc(-c2nc3c(ccc4oc5ccc(N(c6ccc7c(c6)oc6ccccc67)c6ccc7c(c6)sc6ccccc67)cc5c43)o2)cc1]

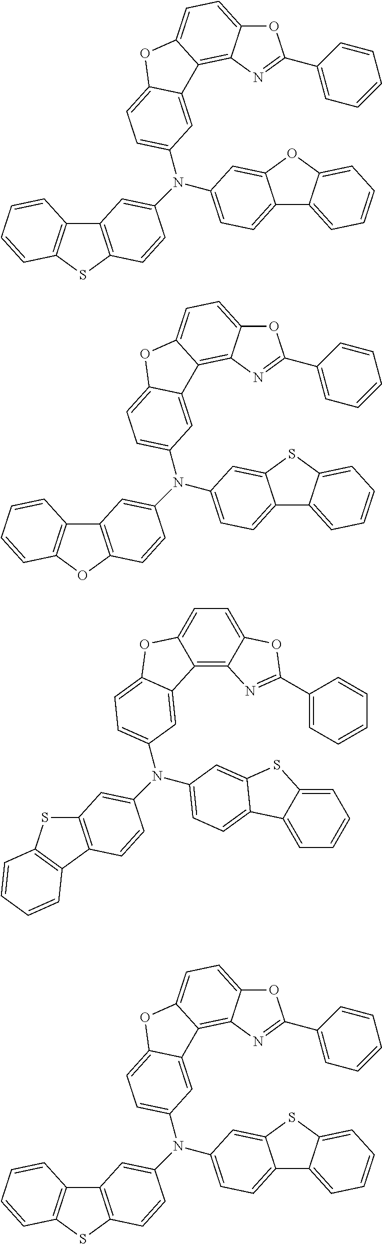 [SMILES: c1ccc(-c2nc3c(ccc4oc5ccc(N(c6ccc7c(c6)oc6ccccc67)c6ccc7sc8ccccc8c7c6)cc5c43)o2)cc1 ; c1ccc(-c2nc3c(ccc4oc5ccc(N(c6ccc7c(c6)sc6ccccc67)c6ccc7oc8ccccc8c7c6)cc5c43)o2)cc1 ; c1ccc(-c2nc3c(ccc4oc5ccc(N(c6ccc7c(c6)sc6ccccc67)c6ccc7c(c6)sc6ccccc67)cc5c43)o2)cc1 ; c1ccc(-c2nc3c(ccc4oc5ccc(N(c6ccc7c(c6)sc6ccccc67)c6ccc7sc8ccccc8c7c6)cc5c43)o2)cc1]

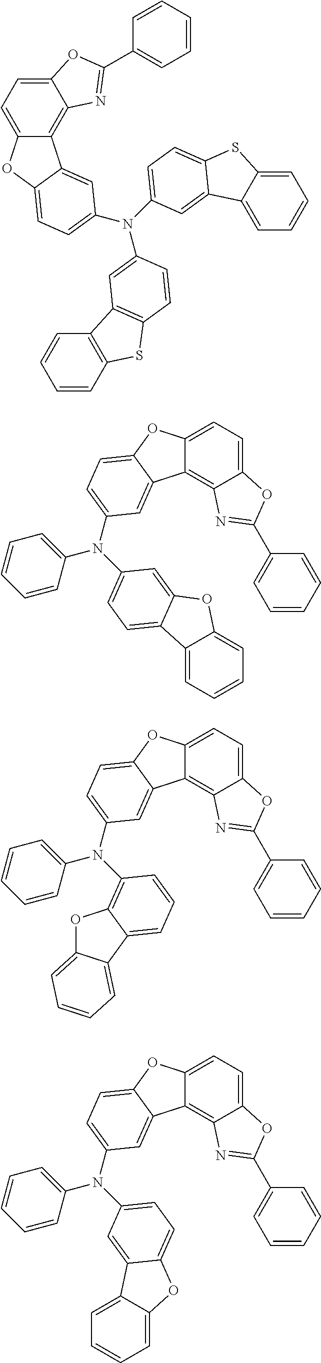

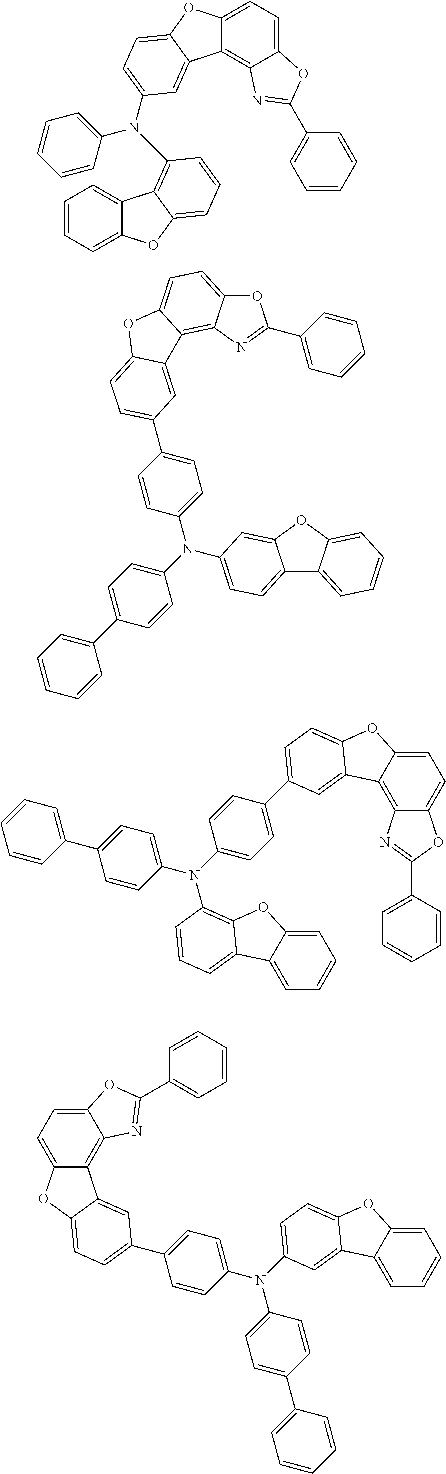

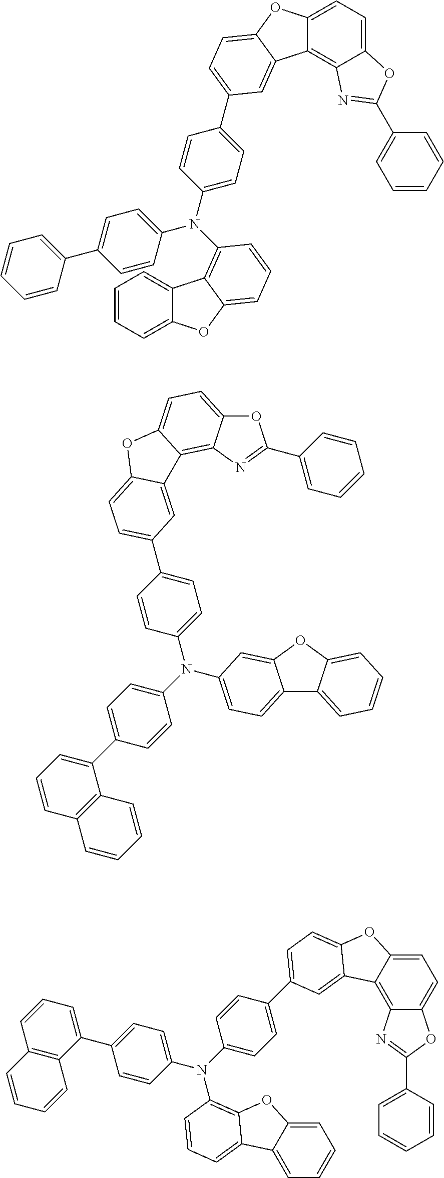 [SMILES: c1ccc(-c2ccc(N(c3ccc(-c4ccc5oc6ccc7oc(-c8ccccc8)nc7c6c5c4)cc3)c3cccc4oc5ccccc5c34)cc2)cc1 ; c1ccc(-c2nc3c(ccc4oc5ccc(-c6ccc(N(c7ccc(-c8cccc9ccccc89)cc7)c7ccc8c(c7)oc7ccccc78)cc6)cc5c43)o2)cc1 ; c1ccc(-c2nc3c(ccc4oc5ccc(-c6ccc(N(c7ccc(-c8cccc9ccccc89)cc7)c7cccc8c7oc7ccccc78)cc6)cc5c43)o2)cc1]

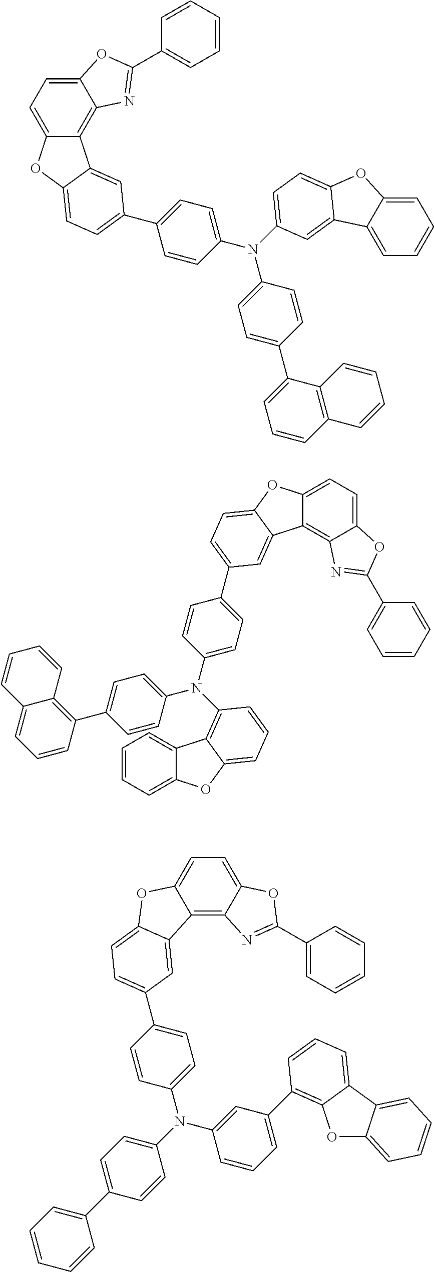

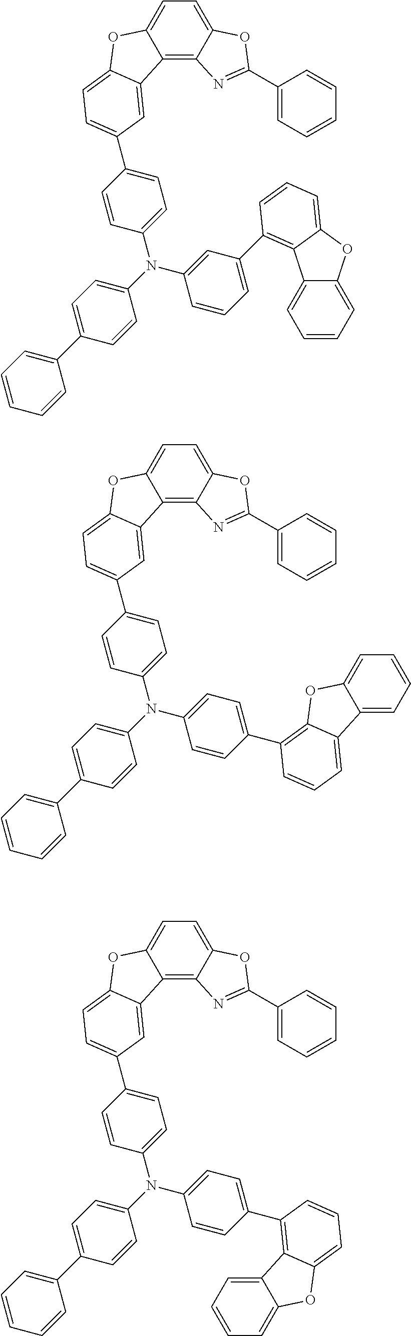 [SMILES: c1ccc(-c2ccc(N(c3ccc(-c4ccc5oc6ccc7oc(-c8ccccc8)nc7c6c5c4)cc3)c3cccc(-c4cccc5oc6ccccc6c45)c3)cc2)cc1 ; c1ccc(-c2ccc(N(c3ccc(-c4ccc5oc6ccc7oc(-c8ccccc8)nc7c6c5c4)cc3)c3ccc(-c4cccc5c4oc4ccccc45)cc3)cc2)cc1 ; c1ccc(-c2ccc(N(c3ccc(-c4ccc5oc6ccc7oc(-c8ccccc8)nc7c6c5c4)cc3)c3ccc(-c4cccc5oc6ccccc6c45)cc3)cc2)cc1]

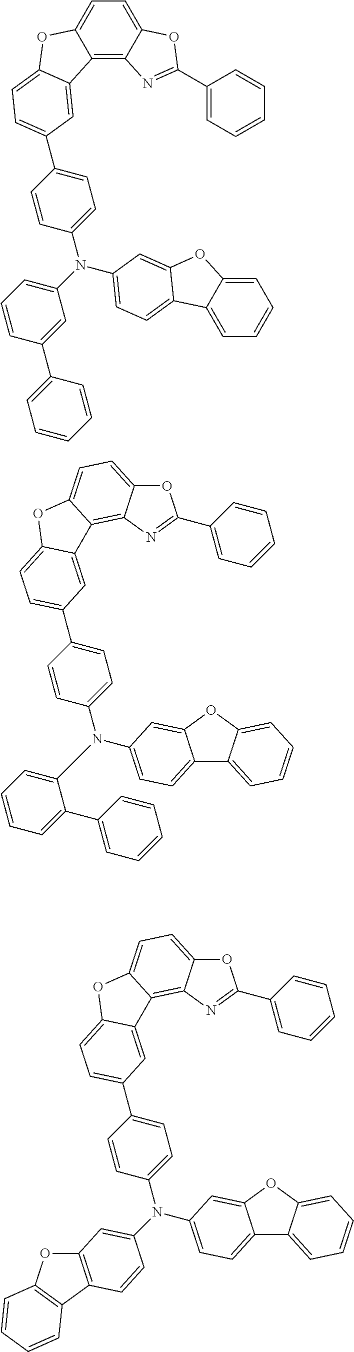

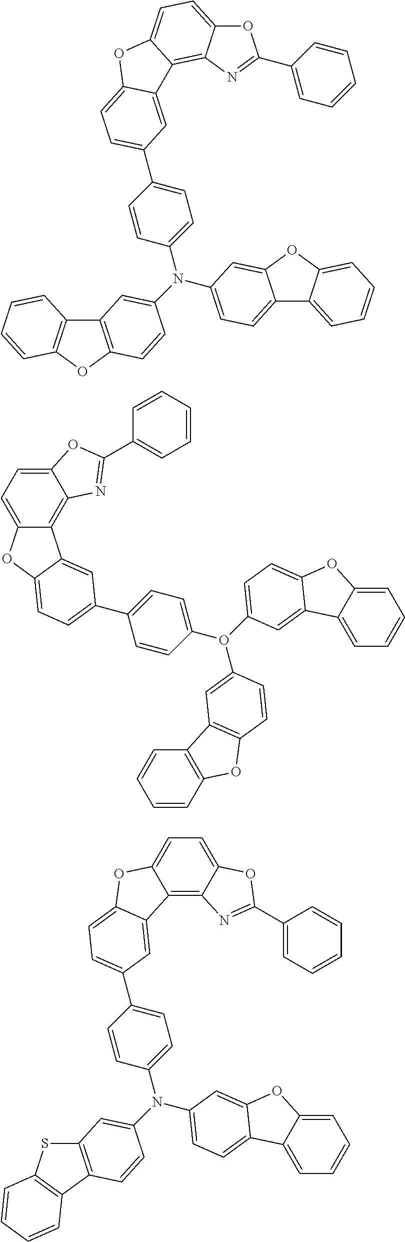

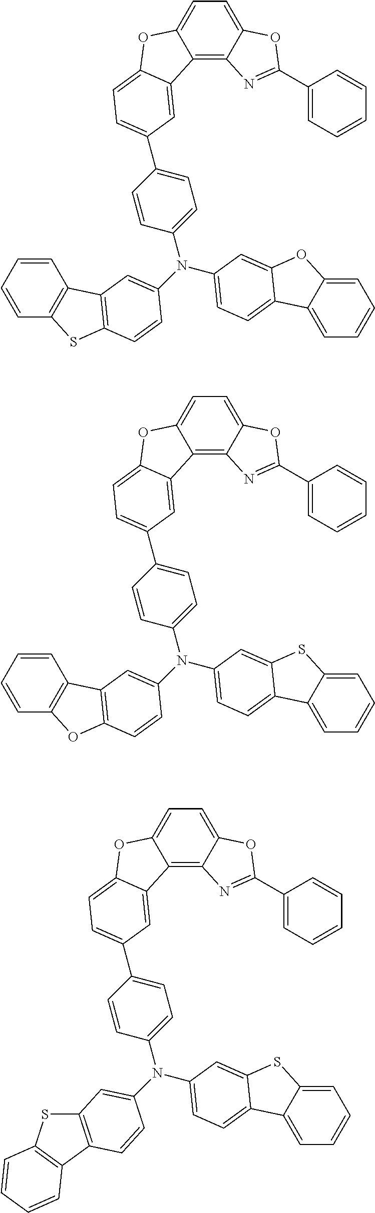 [SMILES: c1ccc(-c2nc3c(ccc4oc5ccc(-c6ccc(N(c7ccc8c(c7)oc7ccccc78)c7ccc8sc9ccccc9c8c7)cc6)cc5c43)o2)cc1 ; c1ccc(-c2nc3c(ccc4oc5ccc(-c6ccc(N(c7ccc8c(c7)sc7ccccc78)c7ccc8oc9ccccc9c8c7)cc6)cc5c43)o2)cc1 ; c1ccc(-c2nc3c(ccc4oc5ccc(-c6ccc(N(c7ccc8c(c7)sc7ccccc78)c7ccc8c(c7)sc7ccccc78)cc6)cc5c43)o2)cc1]

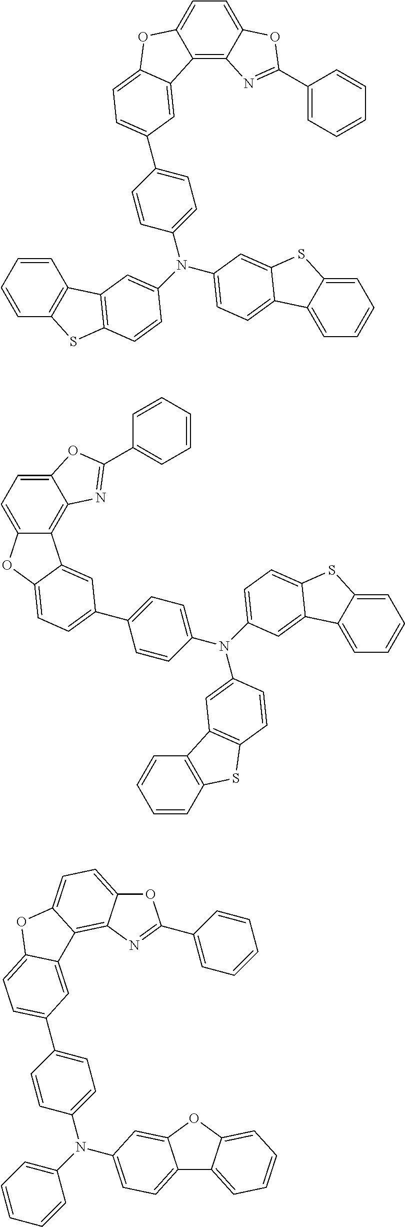 [SMILES: c1ccc(-c2nc3c(ccc4oc5ccc(-c6ccc(N(c7ccc8c(c7)sc7ccccc78)c7ccc8sc9ccccc9c8c7)cc6)cc5c43)o2)cc1 ; c1ccc(-c2nc3c(ccc4oc5ccc(-c6ccc(N(c7ccc8sc9ccccc9c8c7)c7ccc8sc9ccccc9c8c7)cc6)cc5c43)o2)cc1 ; c1ccc(-c2nc3c(ccc4oc5ccc(-c6ccc(N(c7ccccc7)c7ccc8c(c7)oc7ccccc78)cc6)cc5c43)o2)cc1]

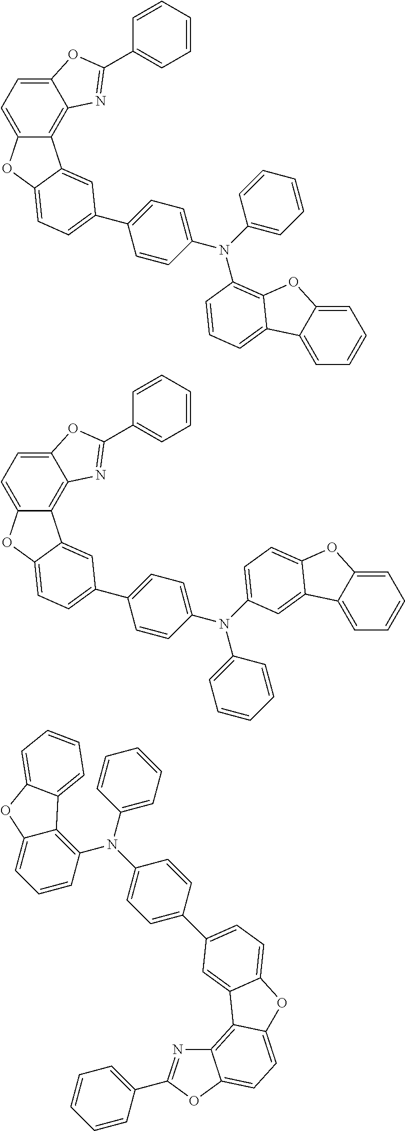 [SMILES: c1ccc(-c2nc3c(ccc4oc5ccc(-c6ccc(N(c7ccccc7)c7cccc8c7oc7ccccc78)cc6)cc5c43)o2)cc1 ; c1ccc(-c2nc3c(ccc4oc5ccc(-c6ccc(N(c7ccccc7)c7ccc8oc9ccccc9c8c7)cc6)cc5c43)o2)cc1 ; c1ccc(-c2nc3c(ccc4oc5ccc(-c6ccc(N(c7ccccc7)c7cccc8oc9ccccc9c78)cc6)cc5c43)o2)cc1]

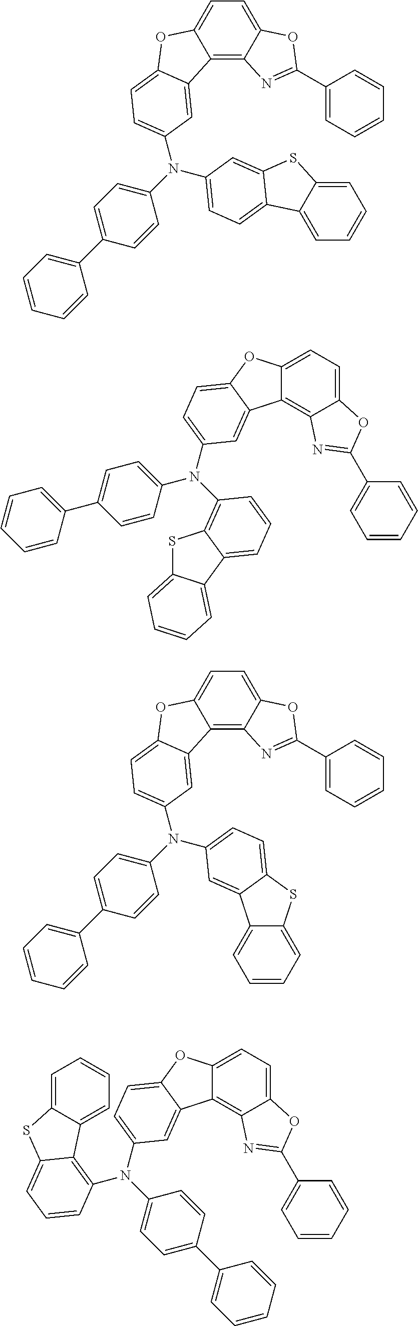

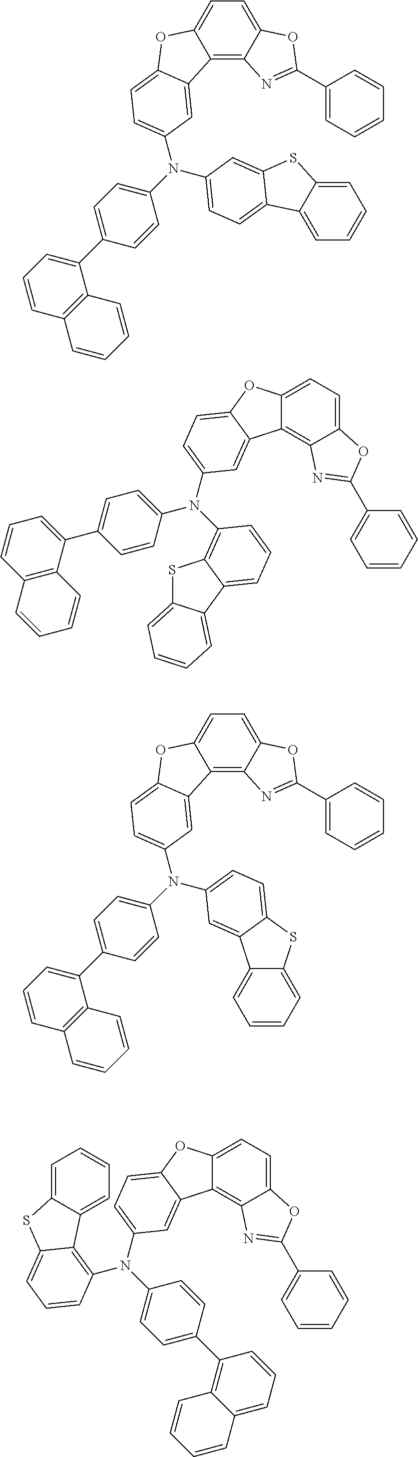 [SMILES: c1ccc(-c2nc3c(ccc4oc5ccc(N(c6ccc(-c7cccc8ccccc78)cc6)c6ccc7c(c6)sc6ccccc67)cc5c43)o2)cc1 ; c1ccc(-c2nc3c(ccc4oc5ccc(N(c6ccc(-c7cccc8ccccc78)cc6)c6cccc7c6sc6ccccc67)cc5c43)o2)cc1 ; c1ccc(-c2nc3c(ccc4oc5ccc(N(c6ccc(-c7cccc8ccccc78)cc6)c6ccc7sc8ccccc8c7c6)cc5c43)o2)cc1 ; c1ccc(-c2nc3c(ccc4oc5ccc(N(c6ccc(-c7cccc8ccccc78)cc6)c6cccc7sc8ccccc8c67)cc5c43)o2)cc1]

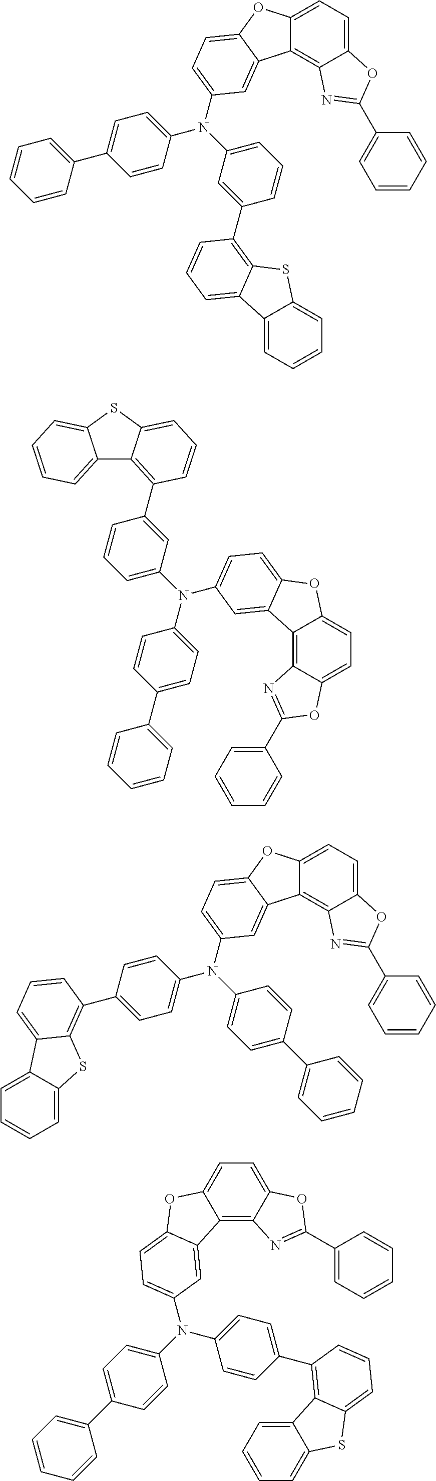

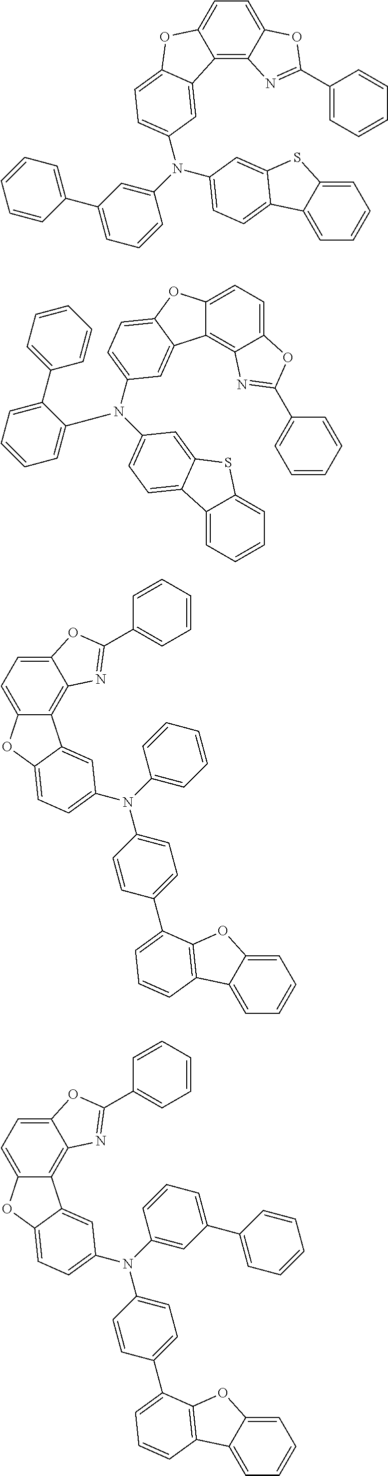

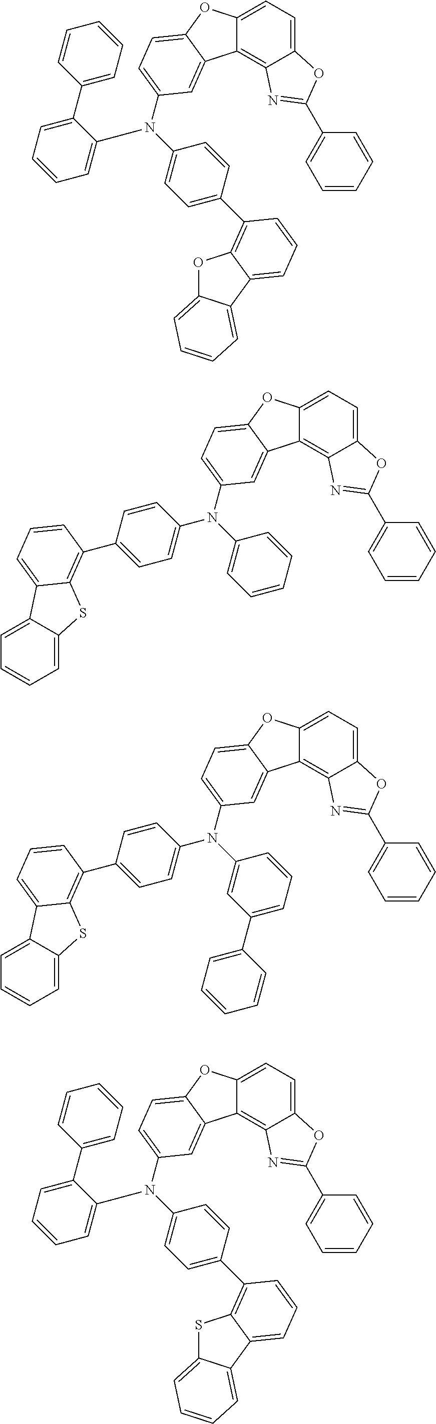 [SMILES: c1ccc(-c2nc3c(ccc4oc5ccc(N(c6ccc(-c7cccc8c7oc7ccccc78)cc6)c6ccccc6-c6ccccc6)cc5c43)o2)cc1 ; c1ccc(-c2nc3c(ccc4oc5ccc(N(c6ccccc6)c6ccc(-c7cccc8c7sc7ccccc78)cc6)cc5c43)o2)cc1 ; c1ccc(-c2cccc(N(c3ccc(-c4cccc5c4sc4ccccc45)cc3)c3ccc4oc5ccc6oc(-c7ccccc7)nc6c5c4c3)c2)cc1 ; c1ccc(-c2nc3c(ccc4oc5ccc(N(c6ccc(-c7cccc8c7sc7ccccc78)cc6)c6ccccc6-c6ccccc6)cc5c43)o2)cc1]

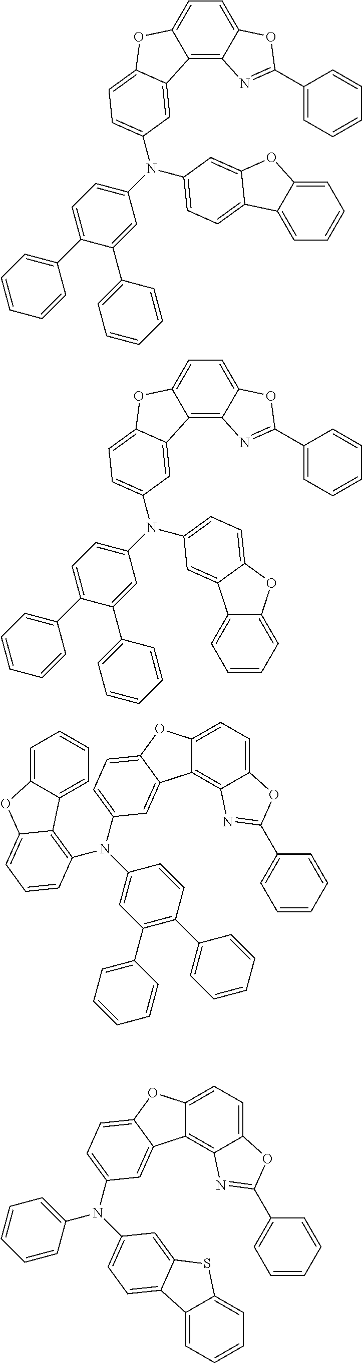

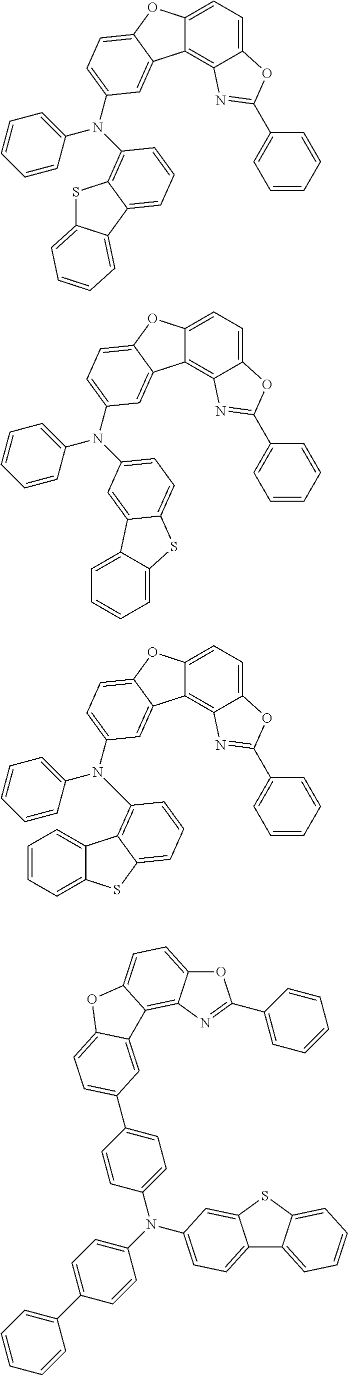 [SMILES: c1ccc(-c2nc3c(ccc4oc5ccc(N(c6ccccc6)c6cccc7c6sc6ccccc67)cc5c43)o2)cc1 ; c1ccc(-c2nc3c(ccc4oc5ccc(N(c6ccccc6)c6ccc7sc8ccccc8c7c6)cc5c43)o2)cc1 ; c1ccc(-c2nc3c(ccc4oc5ccc(N(c6ccccc6)c6cccc7sc8ccccc8c67)cc5c43)o2)cc1 ; c1ccc(-c2ccc(N(c3ccc(-c4ccc5oc6ccc7oc(-c8ccccc8)nc7c6c5c4)cc3)c3ccc4c(c3)sc3ccccc34)cc2)cc1]

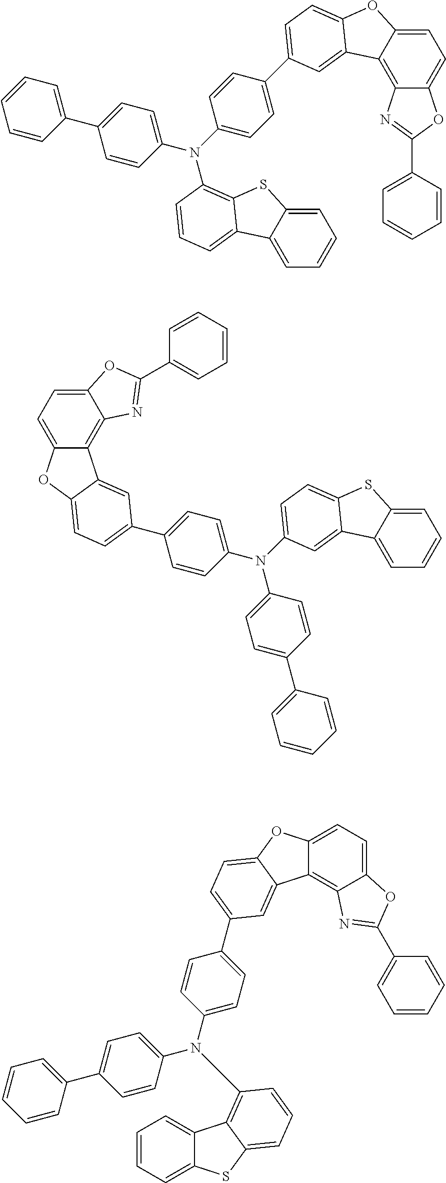 [SMILES: c1ccc(-c2ccc(N(c3ccc(-c4ccc5oc6ccc7oc(-c8ccccc8)nc7c6c5c4)cc3)c3cccc4c3sc3ccccc34)cc2)cc1 ; c1ccc(-c2ccc(N(c3ccc(-c4ccc5oc6ccc7oc(-c8ccccc8)nc7c6c5c4)cc3)c3ccc4sc5ccccc5c4c3)cc2)cc1 ; c1ccc(-c2ccc(N(c3ccc(-c4ccc5oc6ccc7oc(-c8ccccc8)nc7c6c5c4)cc3)c3cccc4sc5ccccc5c34)cc2)cc1]

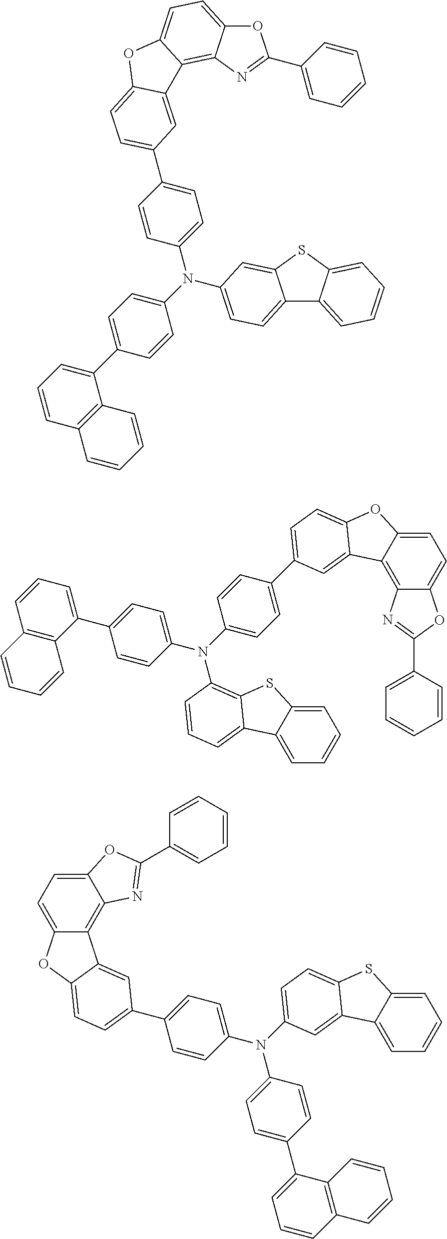 [SMILES: c1ccc(-c2nc3c(ccc4oc5ccc(-c6ccc(N(c7ccc(-c8cccc9ccccc89)cc7)c7ccc8c(c7)sc7ccccc78)cc6)cc5c43)o2)cc1 ; c1ccc(-c2nc3c(ccc4oc5ccc(-c6ccc(N(c7ccc(-c8cccc9ccccc89)cc7)c7cccc8c7sc7ccccc78)cc6)cc5c43)o2)cc1 ; c1ccc(-c2nc3c(ccc4oc5ccc(-c6ccc(N(c7ccc(-c8cccc9ccccc89)cc7)c7ccc8sc9ccccc9c8c7)cc6)cc5c43)o2)cc1]

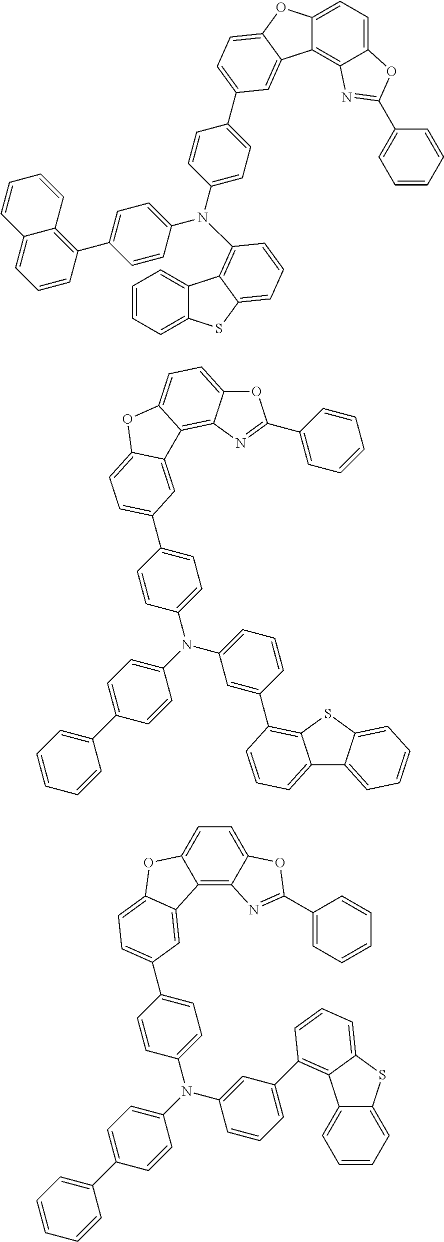

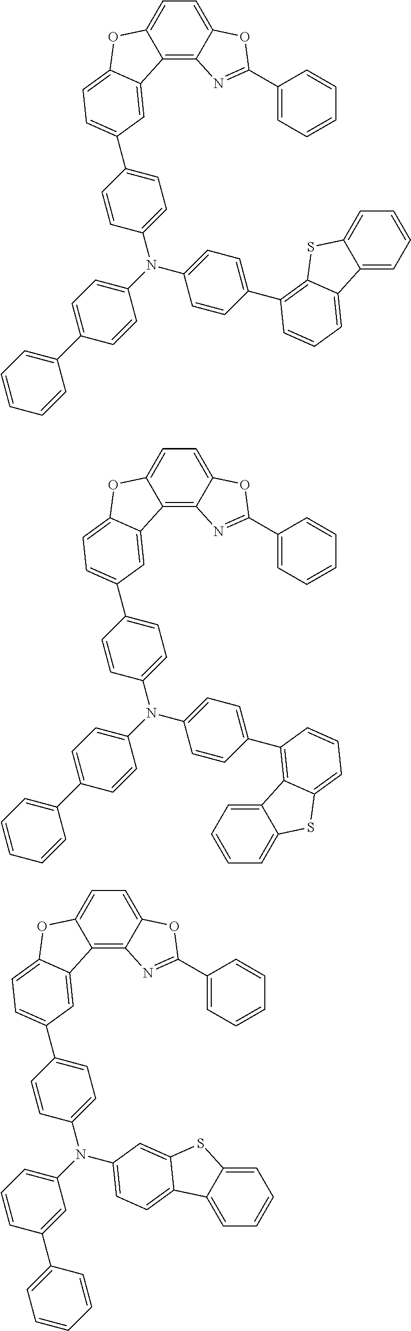 [SMILES: c1ccc(-c2ccc(N(c3ccc(-c4ccc5oc6ccc7oc(-c8ccccc8)nc7c6c5c4)cc3)c3ccc(-c4cccc5c4sc4ccccc45)cc3)cc2)cc1 ; c1ccc(-c2ccc(N(c3ccc(-c4ccc5oc6ccc7oc(-c8ccccc8)nc7c6c5c4)cc3)c3ccc(-c4cccc5sc6ccccc6c45)cc3)cc2)cc1 ; c1ccc(-c2cccc(N(c3ccc(-c4ccc5oc6ccc7oc(-c8ccccc8)nc7c6c5c4)cc3)c3ccc4c(c3)sc3ccccc34)c2)cc1]

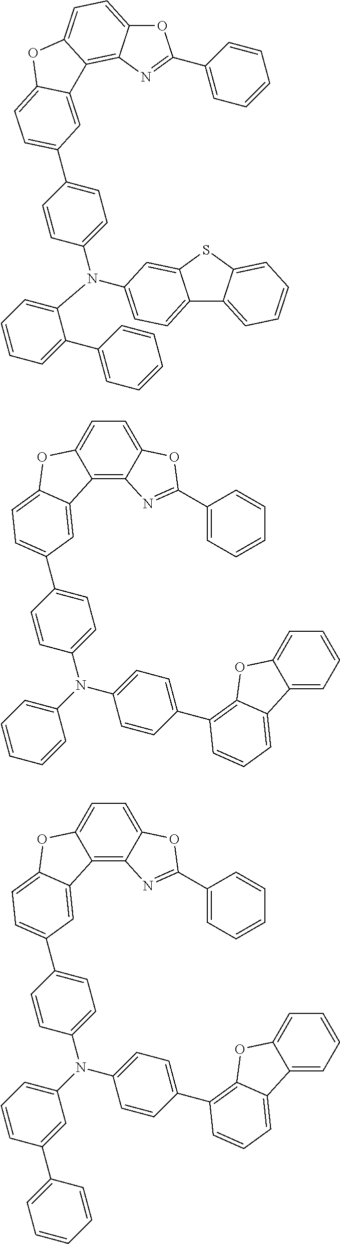

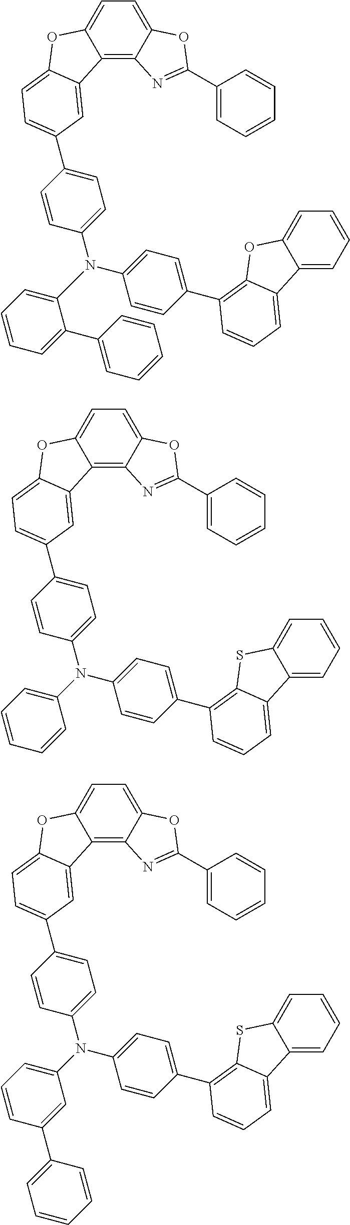

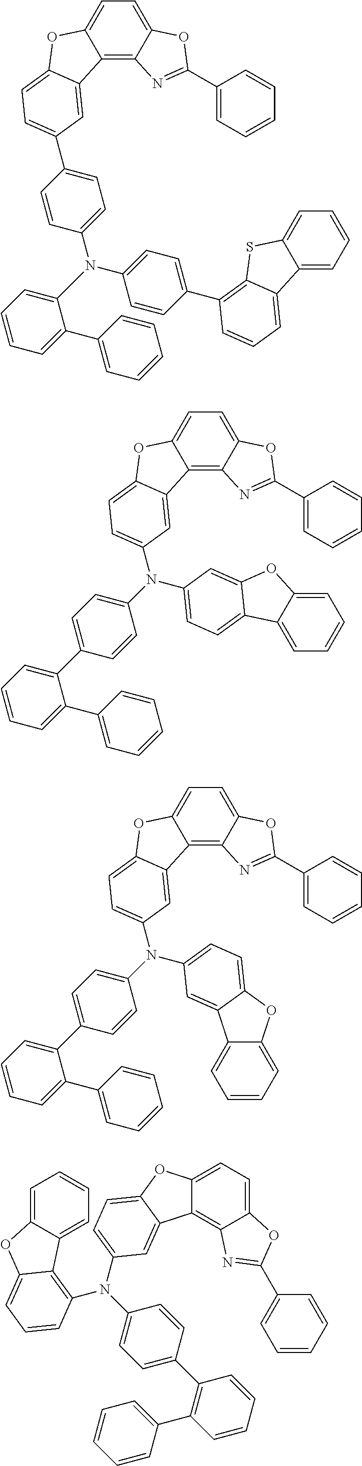

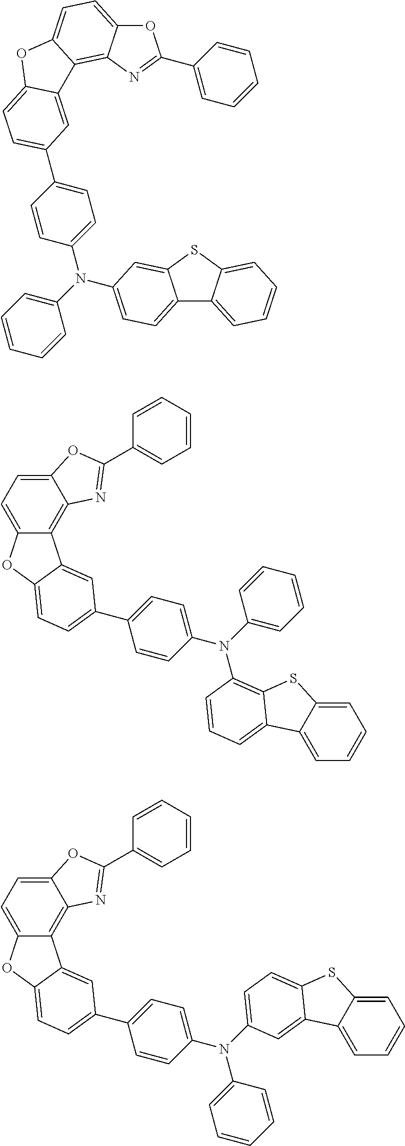 [SMILES: c1ccc(-c2nc3c(ccc4oc5ccc(-c6ccc(N(c7ccccc7)c7ccc8c(c7)sc7ccccc78)cc6)cc5c43)o2)cc1 ; c1ccc(-c2nc3c(ccc4oc5ccc(-c6ccc(N(c7ccccc7)c7cccc8c7sc7ccccc78)cc6)cc5c43)o2)cc1 ; c1ccc(-c2nc3c(ccc4oc5ccc(-c6ccc(N(c7ccccc7)c7ccc8sc9ccccc9c8c7)cc6)cc5c43)o2)cc1]

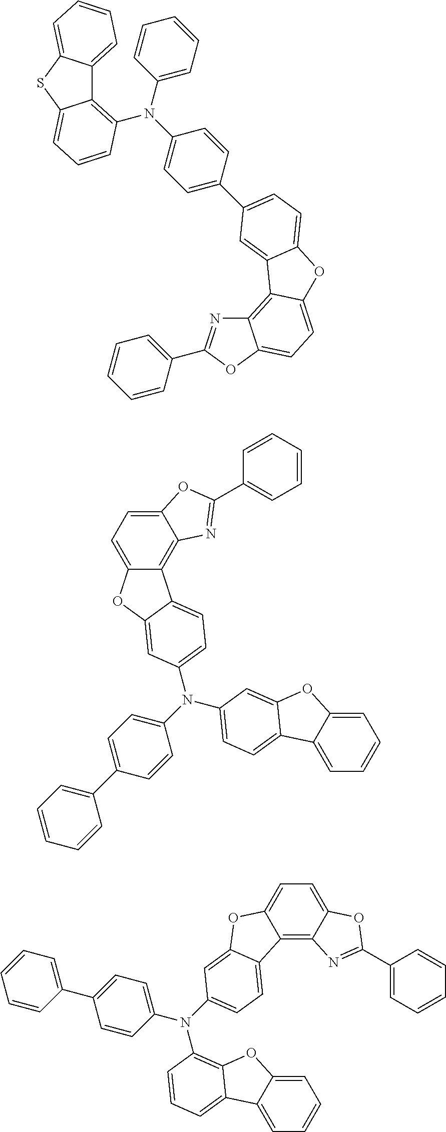 [SMILES: c1ccc(-c2nc3c(ccc4oc5ccc(-c6ccc(N(c7ccccc7)c7cccc8sc9ccccc9c78)cc6)cc5c43)o2)cc1 ; c1ccc(-c2ccc(N(c3ccc4c(c3)oc3ccccc34)c3ccc4c(c3)oc3ccc5oc(-c6ccccc6)nc5c34)cc2)cc1 ; c1ccc(-c2ccc(N(c3ccc4c(c3)oc3ccc5oc(-c6ccccc6)nc5c34)c3cccc4c3oc3ccccc34)cc2)cc1]

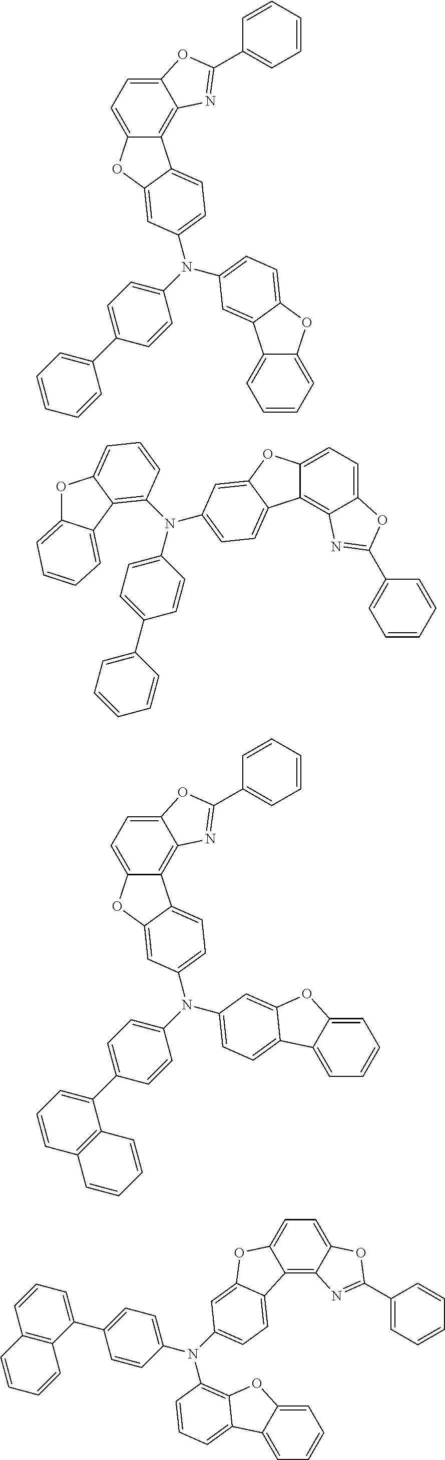

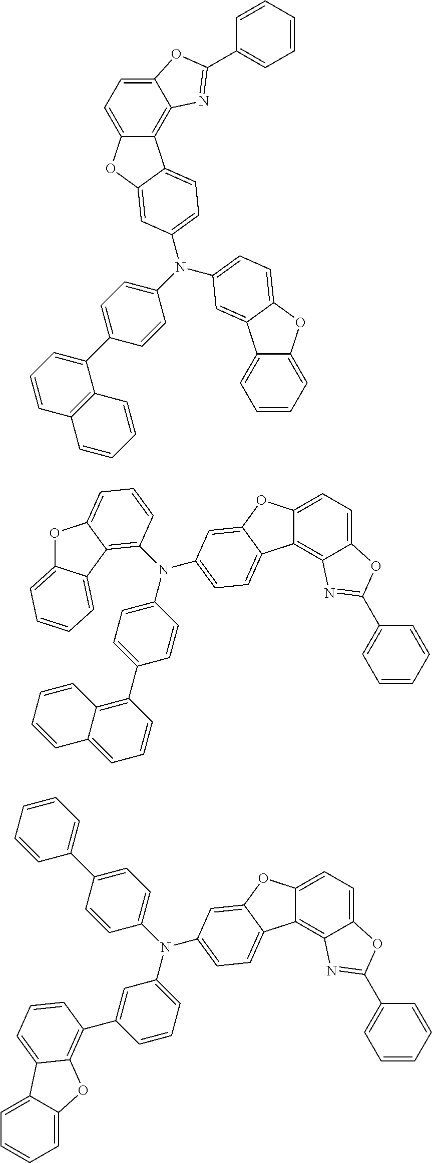

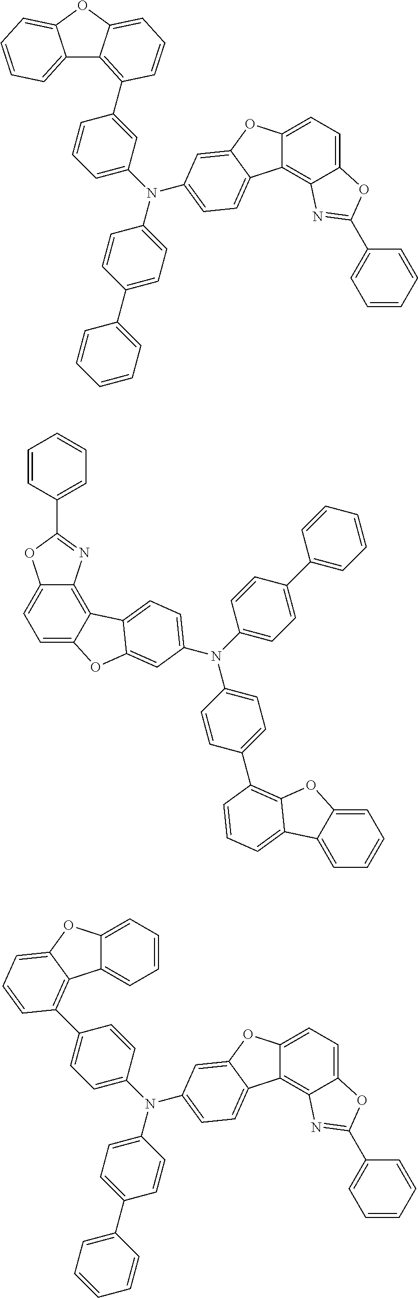 [SMILES: c1ccc(-c2ccc(N(c3cccc(-c4cccc5oc6ccccc6c45)c3)c3ccc4c(c3)oc3ccc5oc(-c6ccccc6)nc5c34)cc2)cc1 ; c1ccc(-c2ccc(N(c3ccc(-c4cccc5c4oc4ccccc45)cc3)c3ccc4c(c3)oc3ccc5oc(-c6ccccc6)nc5c34)cc2)cc1 ; c1ccc(-c2ccc(N(c3ccc(-c4cccc5oc6ccccc6c45)cc3)c3ccc4c(c3)oc3ccc5oc(-c6ccccc6)nc5c34)cc2)cc1]

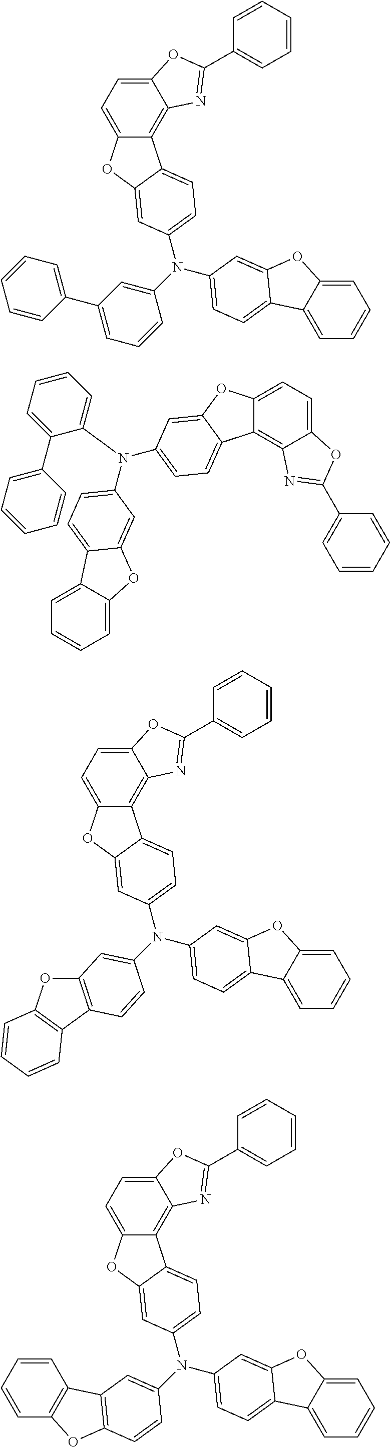 [SMILES: c1ccc(-c2cccc(N(c3ccc4c(c3)oc3ccccc34)c3ccc4c(c3)oc3ccc5oc(-c6ccccc6)nc5c34)c2)cc1 ; c1ccc(-c2nc3c(ccc4oc5cc(N(c6ccc7c(c6)oc6ccccc67)c6ccccc6-c6ccccc6)ccc5c43)o2)cc1 ; c1ccc(-c2nc3c(ccc4oc5cc(N(c6ccc7c(c6)oc6ccccc67)c6ccc7c(c6)oc6ccccc67)ccc5c43)o2)cc1 ; c1ccc(-c2nc3c(ccc4oc5cc(N(c6ccc7c(c6)oc6ccccc67)c6ccc7oc8ccccc8c7c6)ccc5c43)o2)cc1]

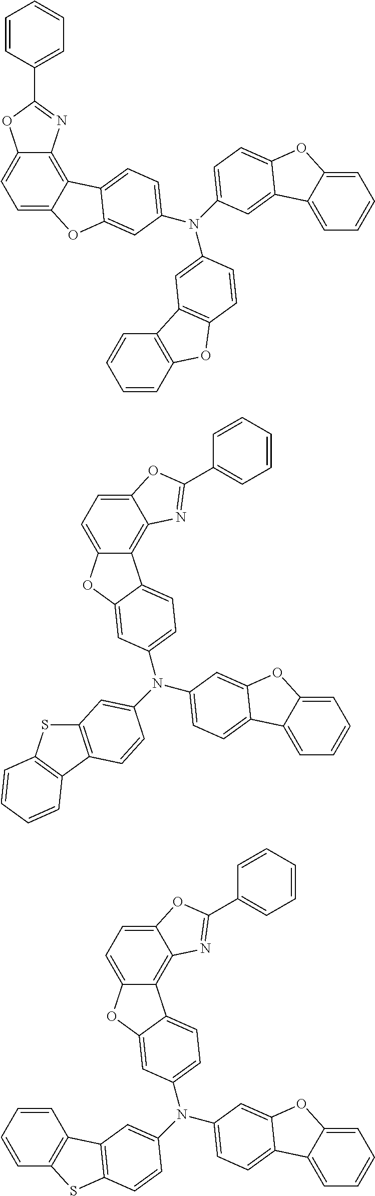 [SMILES: c1ccc(-c2nc3c(ccc4oc5cc(N(c6ccc7oc8ccccc8c7c6)c6ccc7oc8ccccc8c7c6)ccc5c43)o2)cc1 ; c1ccc(-c2nc3c(ccc4oc5cc(N(c6ccc7c(c6)oc6ccccc67)c6ccc7c(c6)sc6ccccc67)ccc5c43)o2)cc1 ; c1ccc(-c2nc3c(ccc4oc5cc(N(c6ccc7c(c6)oc6ccccc67)c6ccc7sc8ccccc8c7c6)ccc5c43)o2)cc1]

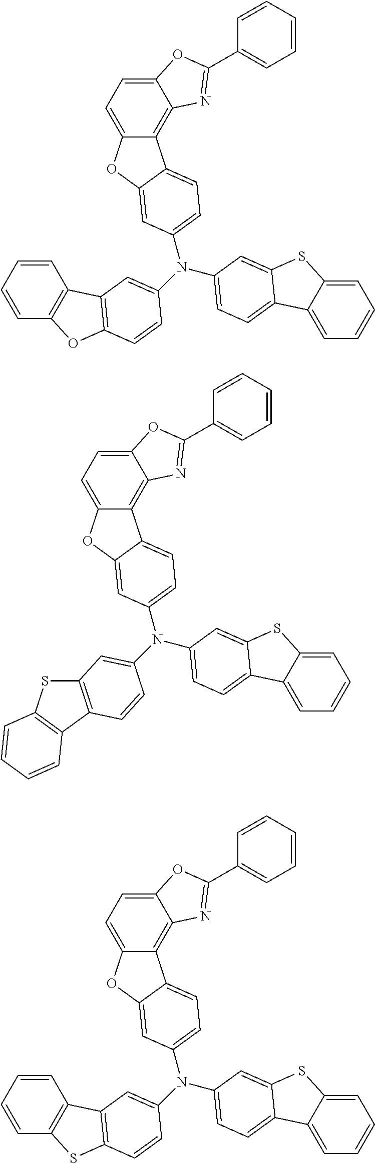 [SMILES: c1ccc(-c2nc3c(ccc4oc5cc(N(c6ccc7c(c6)sc6ccccc67)c6ccc7oc8ccccc8c7c6)ccc5c43)o2)cc1 ; c1ccc(-c2nc3c(ccc4oc5cc(N(c6ccc7c(c6)sc6ccccc67)c6ccc7c(c6)sc6ccccc67)ccc5c43)o2)cc1 ; c1ccc(-c2nc3c(ccc4oc5cc(N(c6ccc7c(c6)sc6ccccc67)c6ccc7sc8ccccc8c7c6)ccc5c43)o2)cc1]

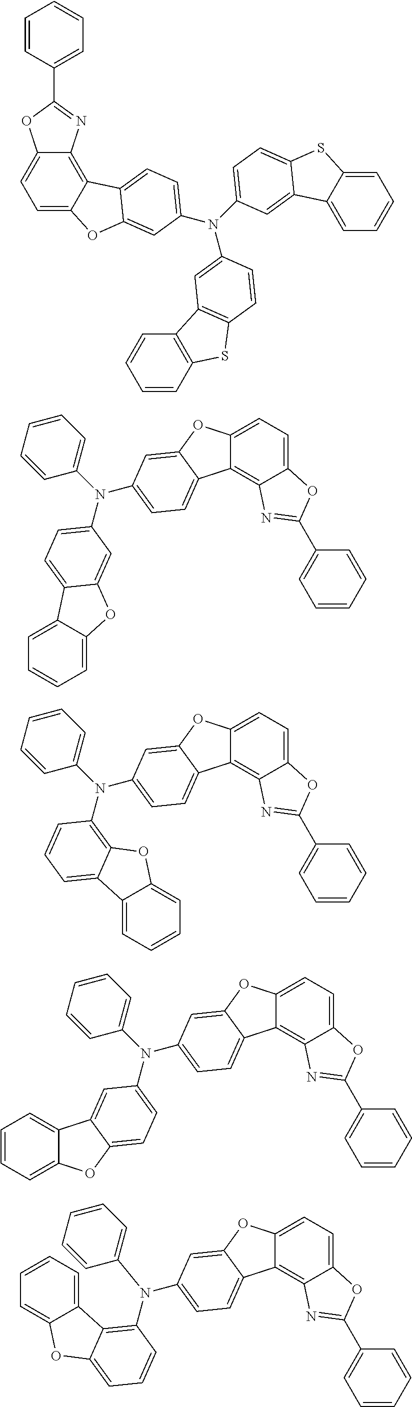

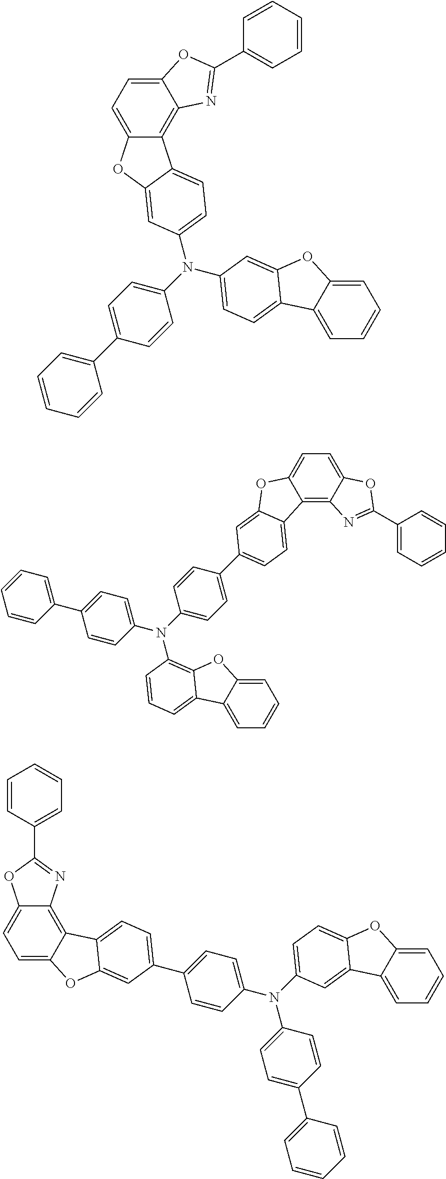 [SMILES: c1ccc(-c2ccc(N(c3ccc4c(c3)oc3ccccc34)c3ccc4c(c3)oc3ccc5oc(-c6ccccc6)nc5c34)cc2)cc1 ; c1ccc(-c2ccc(N(c3ccc(-c4ccc5c(c4)oc4ccc6oc(-c7ccccc7)nc6c45)cc3)c3cccc4c3oc3ccccc34)cc2)cc1 ; c1ccc(-c2ccc(N(c3ccc(-c4ccc5c(c4)oc4ccc6oc(-c7ccccc7)nc6c45)cc3)c3ccc4oc5ccccc5c4c3)cc2)cc1]

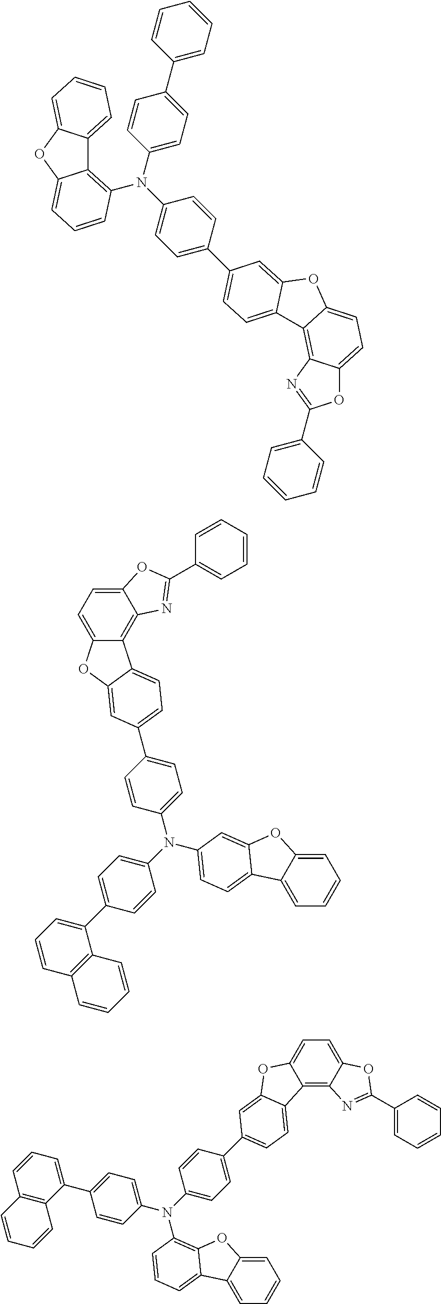 [SMILES: c1ccc(-c2ccc(N(c3ccc(-c4ccc5c(c4)oc4ccc6oc(-c7ccccc7)nc6c45)cc3)c3cccc4oc5ccccc5c34)cc2)cc1 ; c1ccc(-c2nc3c(ccc4oc5cc(-c6ccc(N(c7ccc(-c8cccc9ccccc89)cc7)c7ccc8c(c7)oc7ccccc78)cc6)ccc5c43)o2)cc1 ; c1ccc(-c2nc3c(ccc4oc5cc(-c6ccc(N(c7ccc(-c8cccc9ccccc89)cc7)c7cccc8c7oc7ccccc78)cc6)ccc5c43)o2)cc1]

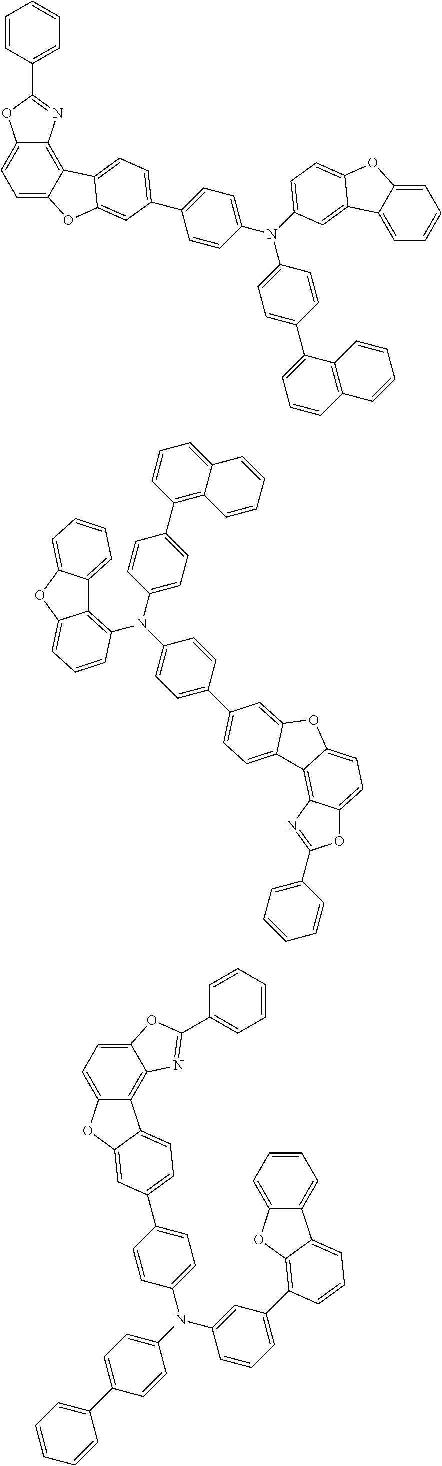 [SMILES: c1ccc(-c2nc3c(ccc4oc5cc(-c6ccc(N(c7ccc(-c8cccc9ccccc89)cc7)c7ccc8oc9ccccc9c8c7)cc6)ccc5c43)o2)cc1 ; c1ccc(-c2nc3c(ccc4oc5cc(-c6ccc(N(c7ccc(-c8cccc9ccccc89)cc7)c7cccc8oc9ccccc9c78)cc6)ccc5c43)o2)cc1 ; c1ccc(-c2ccc(N(c3ccc(-c4ccc5c(c4)oc4ccc6oc(-c7ccccc7)nc6c45)cc3)c3cccc(-c4cccc5c4oc4ccccc45)c3)cc2)cc1]

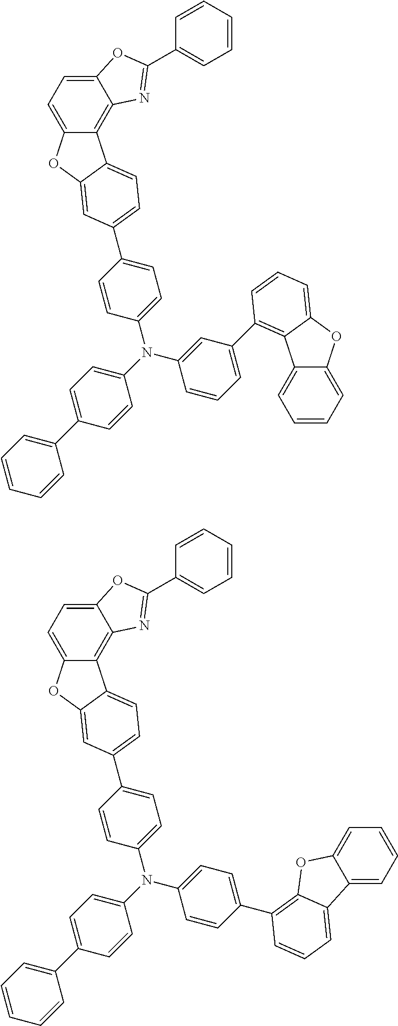 [SMILES: c1ccc(-c2ccc(N(c3ccc(-c4ccc5c(c4)oc4ccc6oc(-c7ccccc7)nc6c45)cc3)c3cccc(-c4cccc5oc6ccccc6c45)c3)cc2)cc1 ; c1ccc(-c2ccc(N(c3ccc(-c4ccc5c(c4)oc4ccc6oc(-c7ccccc7)nc6c45)cc3)c3ccc(-c4cccc5c4oc4ccccc45)cc3)cc2)cc1]

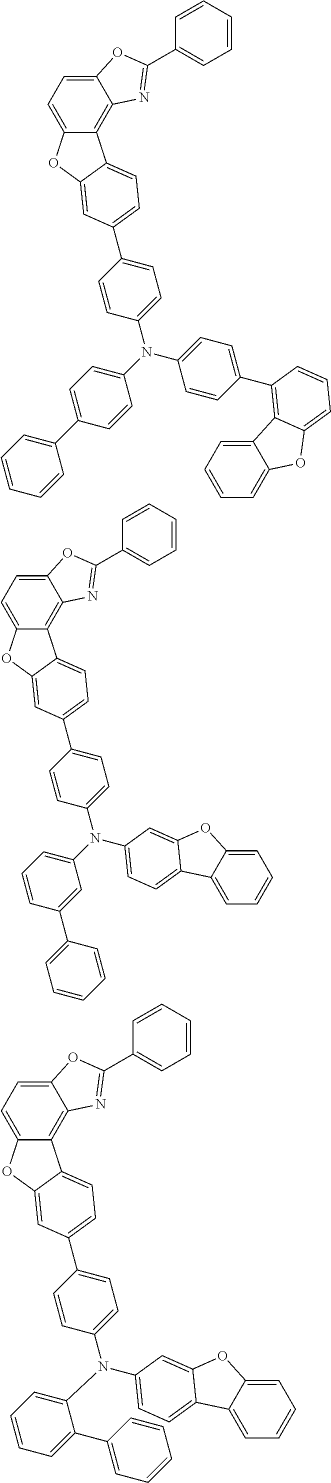 [SMILES: c1ccc(-c2ccc(N(c3ccc(-c4ccc5c(c4)oc4ccc6oc(-c7ccccc7)nc6c45)cc3)c3ccc(-c4cccc5oc6ccccc6c45)cc3)cc2)cc1 ; c1ccc(-c2cccc(N(c3ccc(-c4ccc5c(c4)oc4ccc6oc(-c7ccccc7)nc6c45)cc3)c3ccc4c(c3)oc3ccccc34)c2)cc1 ; c1ccc(-c2nc3c(ccc4oc5cc(-c6ccc(N(c7ccc8c(c7)oc7ccccc78)c7ccccc7-c7ccccc7)cc6)ccc5c43)o2)cc1]

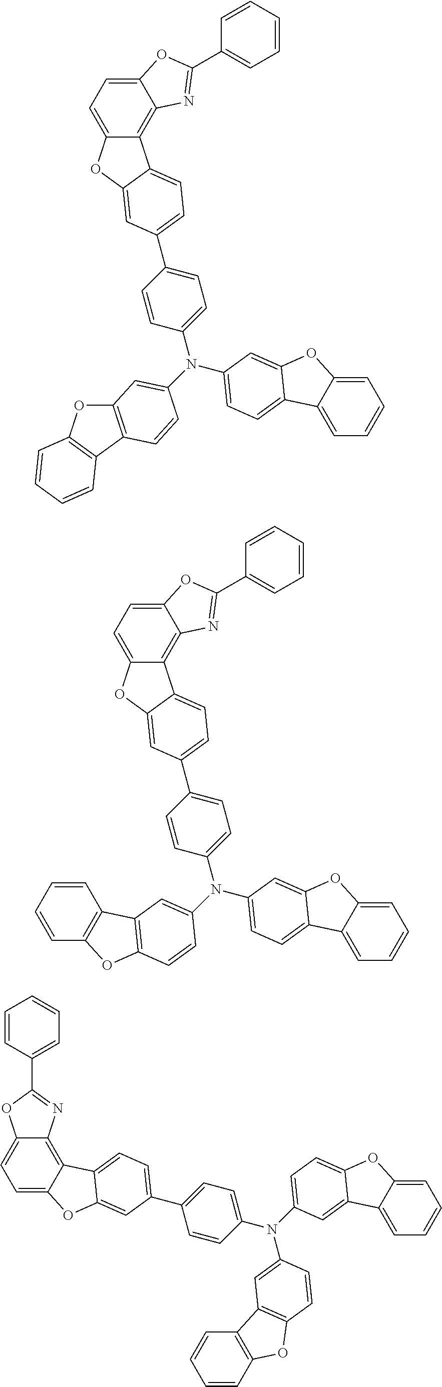 [SMILES: c1ccc(-c2nc3c(ccc4oc5cc(-c6ccc(N(c7ccc8c(c7)oc7ccccc78)c7ccc8c(c7)oc7ccccc78)cc6)ccc5c43)o2)cc1 ; c1ccc(-c2nc3c(ccc4oc5cc(-c6ccc(N(c7ccc8c(c7)oc7ccccc78)c7ccc8oc9ccccc9c8c7)cc6)ccc5c43)o2)cc1 ; c1ccc(-c2nc3c(ccc4oc5cc(-c6ccc(N(c7ccc8oc9ccccc9c8c7)c7ccc8oc9ccccc9c8c7)cc6)ccc5c43)o2)cc1]

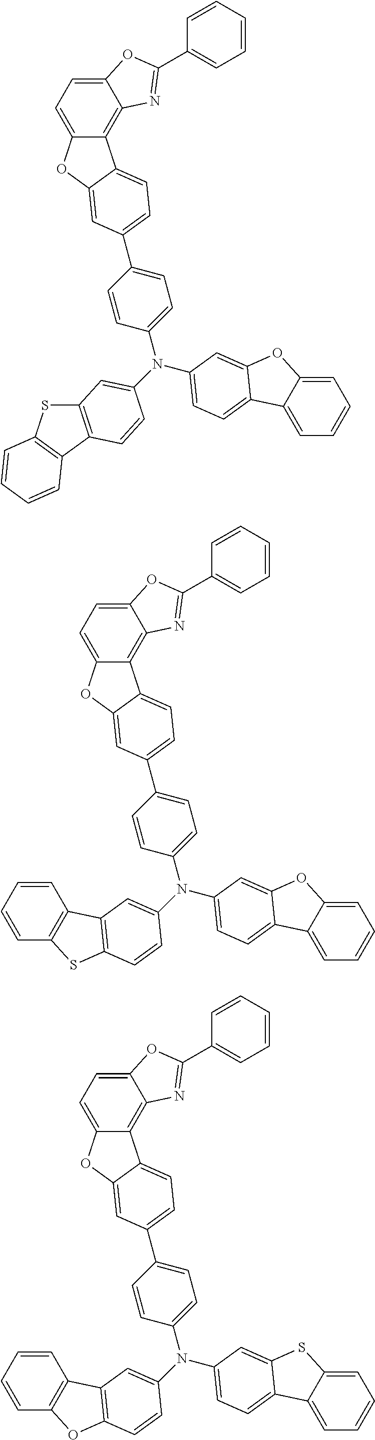 [SMILES: c1ccc(-c2nc3c(ccc4oc5cc(-c6ccc(N(c7ccc8c(c7)oc7ccccc78)c7ccc8c(c7)sc7ccccc78)cc6)ccc5c43)o2)cc1 ; c1ccc(-c2nc3c(ccc4oc5cc(-c6ccc(N(c7ccc8c(c7)oc7ccccc78)c7ccc8sc9ccccc9c8c7)cc6)ccc5c43)o2)cc1 ; c1ccc(-c2nc3c(ccc4oc5cc(-c6ccc(N(c7ccc8c(c7)sc7ccccc78)c7ccc8oc9ccccc9c8c7)cc6)ccc5c43)o2)cc1]

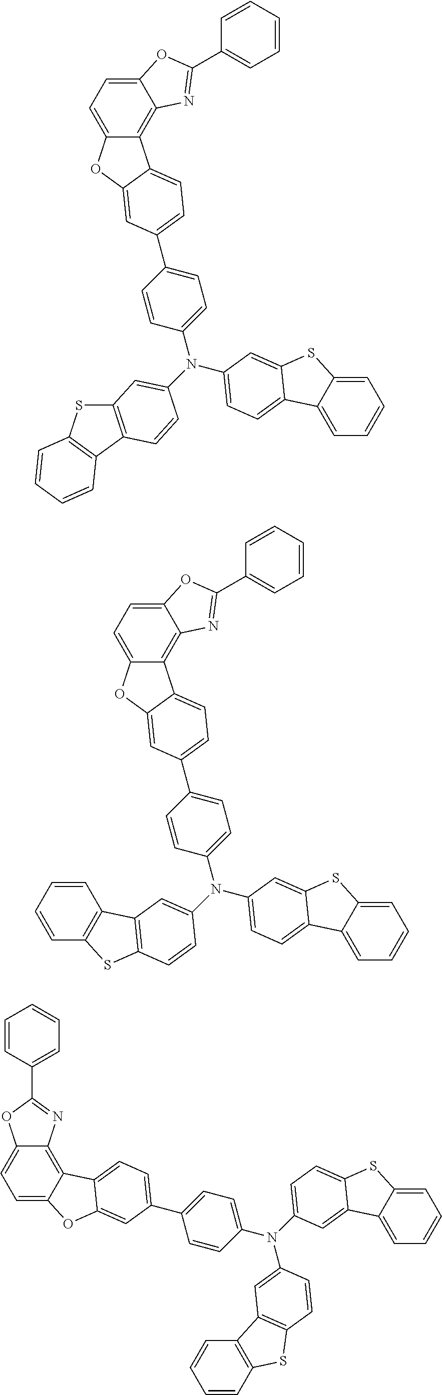 [SMILES: c1ccc(-c2nc3c(ccc4oc5cc(-c6ccc(N(c7ccc8c(c7)sc7ccccc78)c7ccc8c(c7)sc7ccccc78)cc6)ccc5c43)o2)cc1 ; c1ccc(-c2nc3c(ccc4oc5cc(-c6ccc(N(c7ccc8c(c7)sc7ccccc78)c7ccc8sc9ccccc9c8c7)cc6)ccc5c43)o2)cc1 ; c1ccc(-c2nc3c(ccc4oc5cc(-c6ccc(N(c7ccc8sc9ccccc9c8c7)c7ccc8sc9ccccc9c8c7)cc6)ccc5c43)o2)cc1]

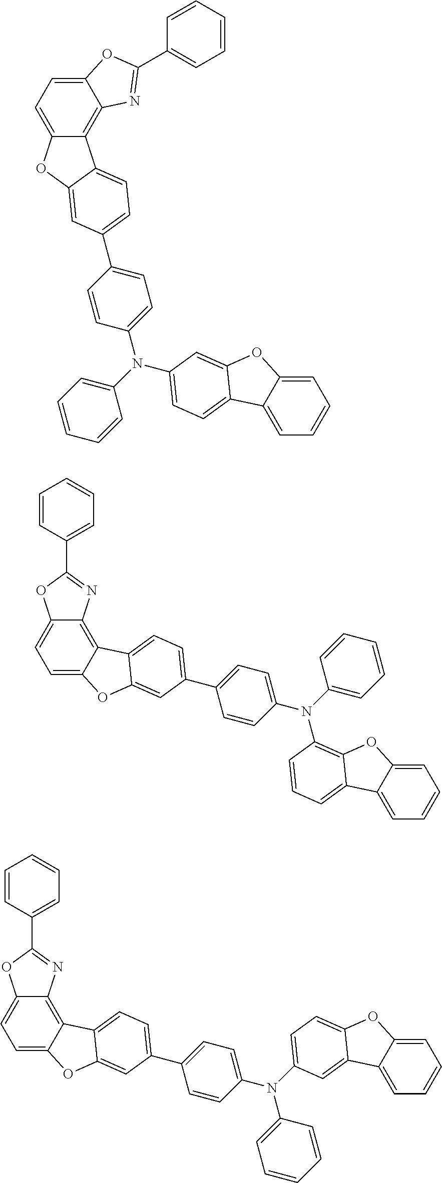 [SMILES: c1ccc(-c2nc3c(ccc4oc5cc(-c6ccc(N(c7ccccc7)c7ccc8c(c7)oc7ccccc78)cc6)ccc5c43)o2)cc1 ; c1ccc(-c2nc3c(ccc4oc5cc(-c6ccc(N(c7ccccc7)c7cccc8c7oc7ccccc78)cc6)ccc5c43)o2)cc1 ; c1ccc(-c2nc3c(ccc4oc5cc(-c6ccc(N(c7ccccc7)c7ccc8oc9ccccc9c8c7)cc6)ccc5c43)o2)cc1]

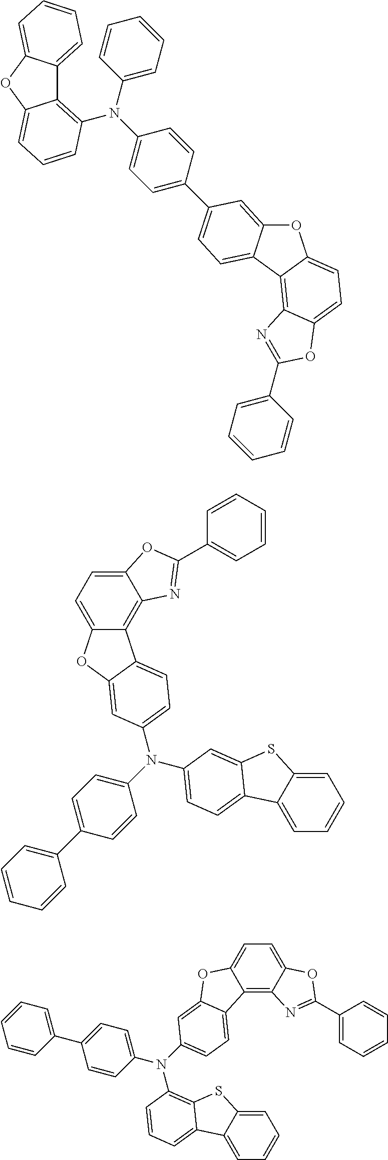

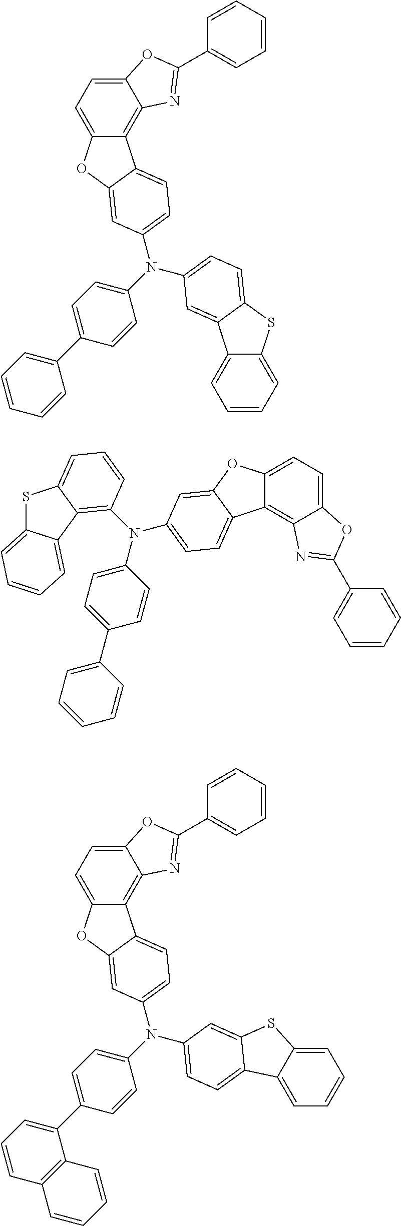 [SMILES: c1ccc(-c2ccc(N(c3ccc4c(c3)oc3ccc5oc(-c6ccccc6)nc5c34)c3ccc4sc5ccccc5c4c3)cc2)cc1 ; c1ccc(-c2ccc(N(c3ccc4c(c3)oc3ccc5oc(-c6ccccc6)nc5c34)c3cccc4sc5ccccc5c34)cc2)cc1 ; c1ccc(-c2nc3c(ccc4oc5cc(N(c6ccc(-c7cccc8ccccc78)cc6)c6ccc7c(c6)sc6ccccc67)ccc5c43)o2)cc1]

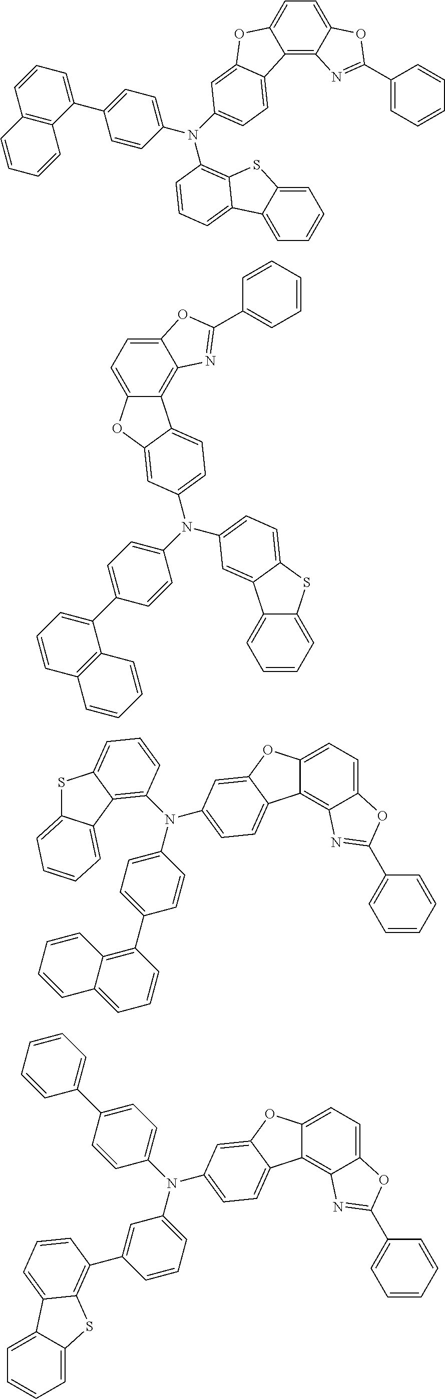

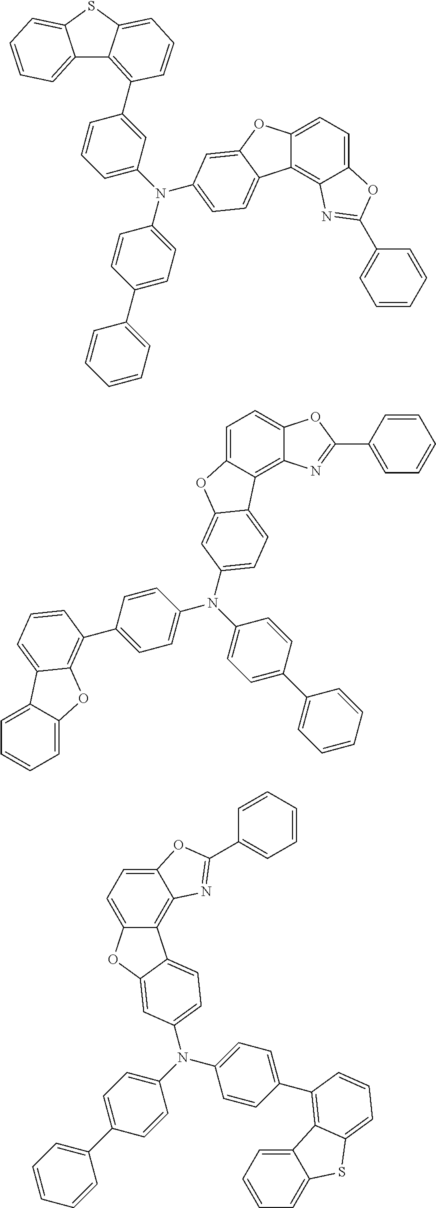 [SMILES: c1ccc(-c2ccc(N(c3cccc(-c4cccc5sc6ccccc6c45)c3)c3ccc4c(c3)oc3ccc5oc(-c6ccccc6)nc5c34)cc2)cc1 ; c1ccc(-c2ccc(N(c3ccc(-c4cccc5c4oc4ccccc45)cc3)c3ccc4c(c3)oc3ccc5oc(-c6ccccc6)nc5c34)cc2)cc1 ; c1ccc(-c2ccc(N(c3ccc(-c4cccc5sc6ccccc6c45)cc3)c3ccc4c(c3)oc3ccc5oc(-c6ccccc6)nc5c34)cc2)cc1]

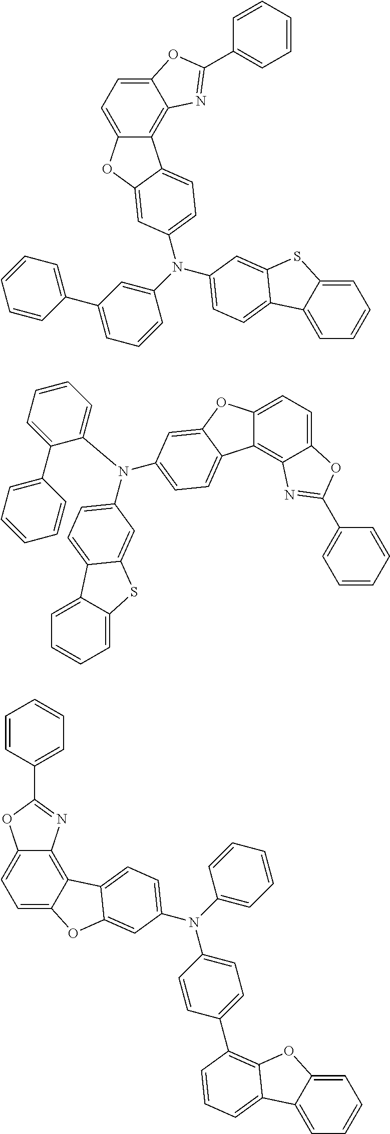 [SMILES: c1ccc(-c2cccc(N(c3ccc4c(c3)oc3ccc5oc(-c6ccccc6)nc5c34)c3ccc4c(c3)sc3ccccc34)c2)cc1 ; c1ccc(-c2nc3c(ccc4oc5cc(N(c6ccc7c(c6)sc6ccccc67)c6ccccc6-c6ccccc6)ccc5c43)o2)cc1 ; c1ccc(-c2nc3c(ccc4oc5cc(N(c6ccccc6)c6ccc(-c7cccc8c7oc7ccccc78)cc6)ccc5c43)o2)cc1]

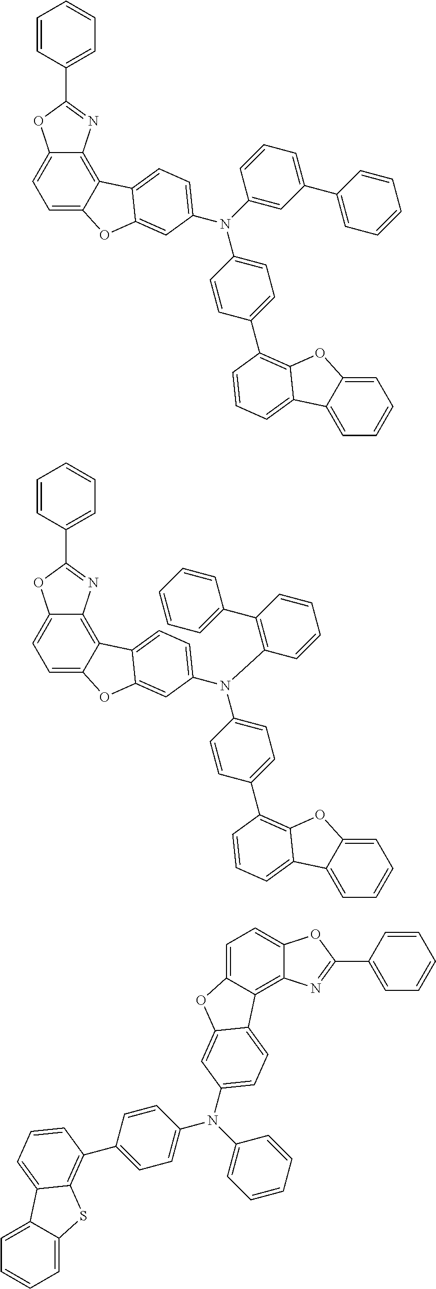

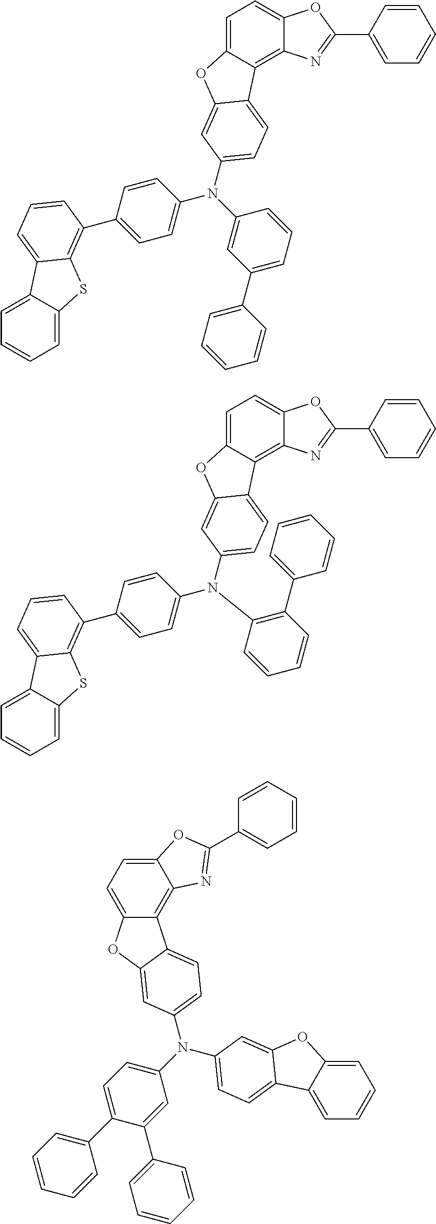

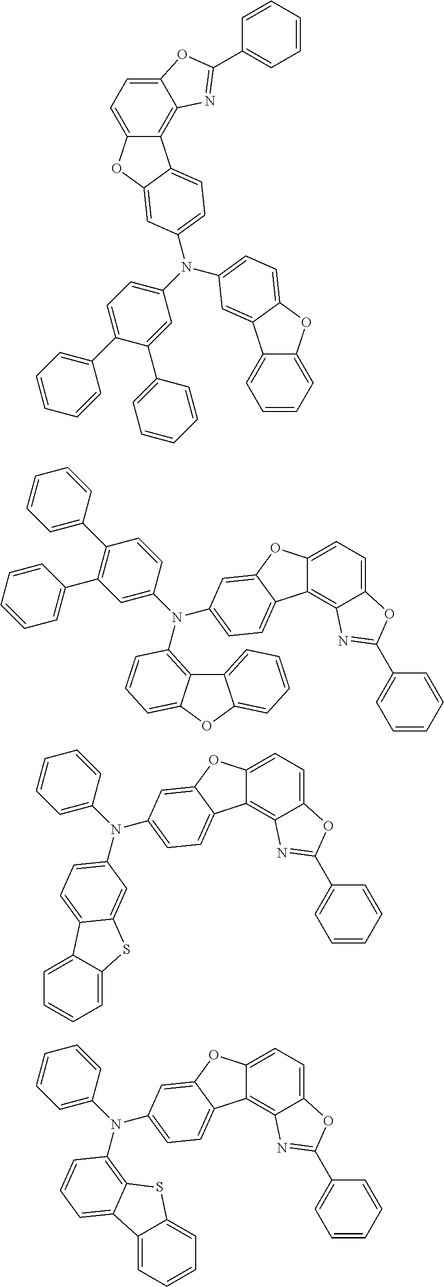

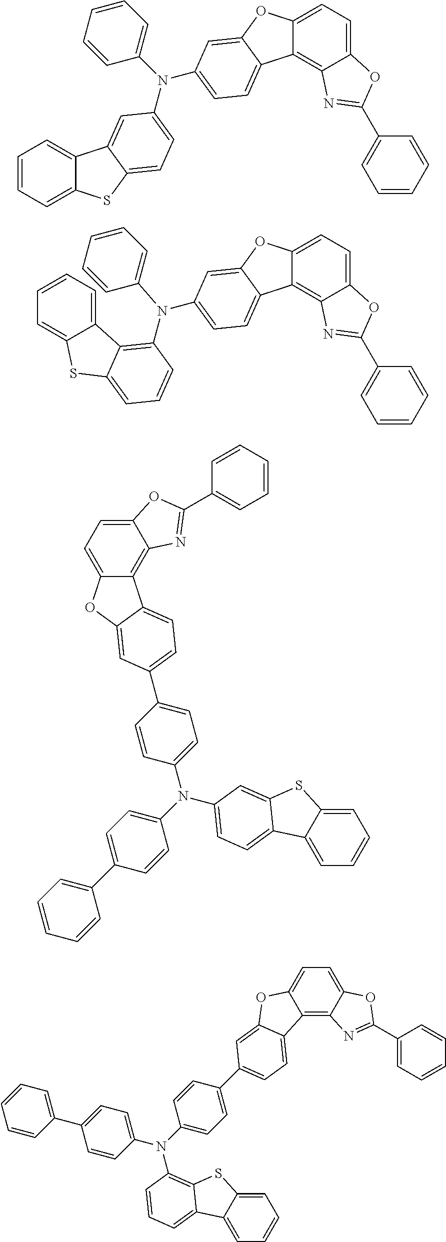

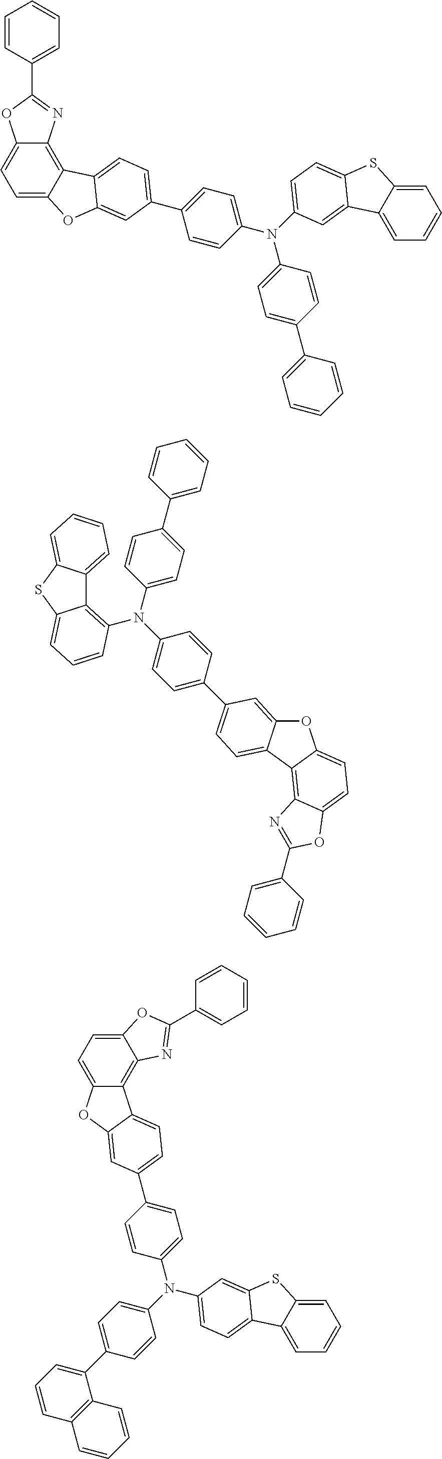 [SMILES: c1ccc(-c2ccc(N(c3ccc(-c4ccc5c(c4)oc4ccc6oc(-c7ccccc7)nc6c45)cc3)c3ccc4sc5ccccc5c4c3)cc2)cc1 ; c1ccc(-c2ccc(N(c3ccc(-c4ccc5c(c4)oc4ccc6oc(-c7ccccc7)nc6c45)cc3)c3cccc4sc5ccccc5c34)cc2)cc1 ; c1ccc(-c2nc3c(ccc4oc5cc(-c6ccc(N(c7ccc(-c8cccc9ccccc89)cc7)c7ccc8c(c7)sc7ccccc78)cc6)ccc5c43)o2)cc1]

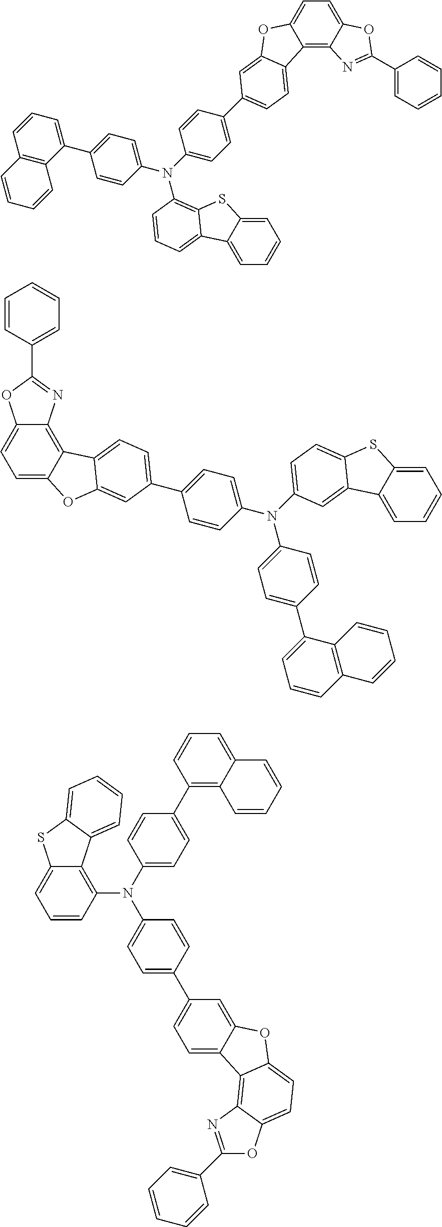 [SMILES: c1ccc(-c2nc3c(ccc4oc5cc(-c6ccc(N(c7ccc(-c8cccc9ccccc89)cc7)c7cccc8c7sc7ccccc78)cc6)ccc5c43)o2)cc1 ; c1ccc(-c2nc3c(ccc4oc5cc(-c6ccc(N(c7ccc(-c8cccc9ccccc89)cc7)c7ccc8sc9ccccc9c8c7)cc6)ccc5c43)o2)cc1 ; c1ccc(-c2nc3c(ccc4oc5cc(-c6ccc(N(c7ccc(-c8cccc9ccccc89)cc7)c7cccc8sc9ccccc9c78)cc6)ccc5c43)o2)cc1]

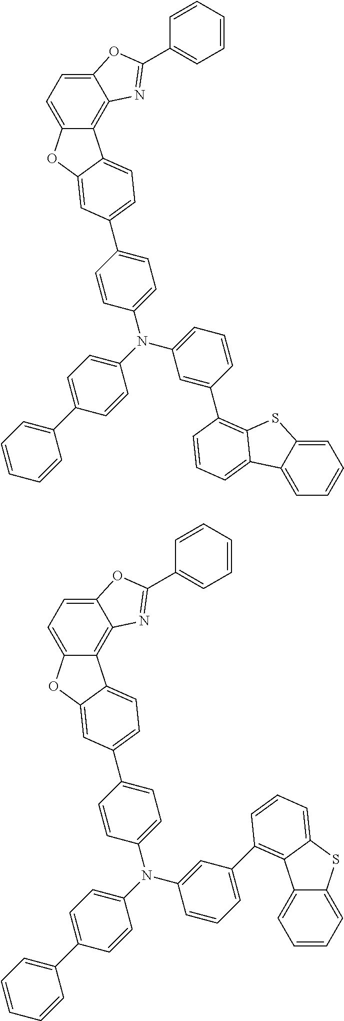 [SMILES: c1ccc(-c2ccc(N(c3ccc(-c4ccc5c(c4)oc4ccc6oc(-c7ccccc7)nc6c45)cc3)c3cccc(-c4cccc5c4sc4ccccc45)c3)cc2)cc1 ; c1ccc(-c2ccc(N(c3ccc(-c4ccc5c(c4)oc4ccc6oc(-c7ccccc7)nc6c45)cc3)c3cccc(-c4cccc5sc6ccccc6c45)c3)cc2)cc1]

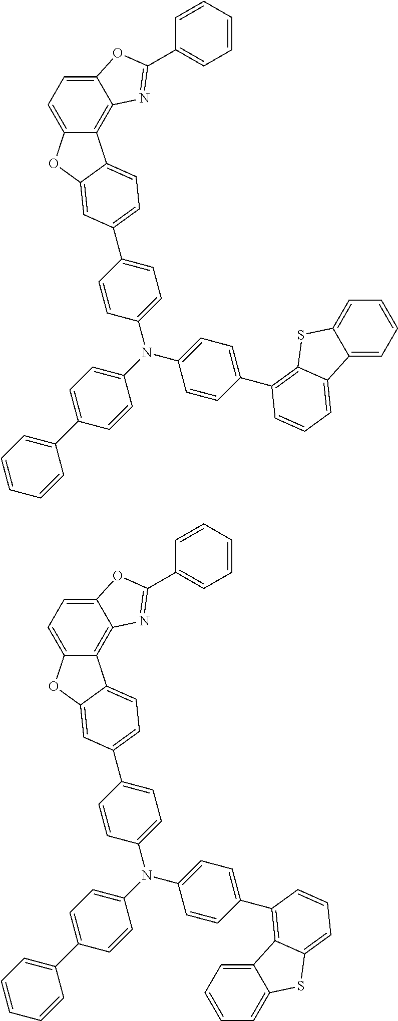 [SMILES: c1ccc(-c2ccc(N(c3ccc(-c4ccc5c(c4)oc4ccc6oc(-c7ccccc7)nc6c45)cc3)c3ccc(-c4cccc5c4sc4ccccc45)cc3)cc2)cc1 ; c1ccc(-c2ccc(N(c3ccc(-c4ccc5c(c4)oc4ccc6oc(-c7ccccc7)nc6c45)cc3)c3ccc(-c4cccc5sc6ccccc6c45)cc3)cc2)cc1]

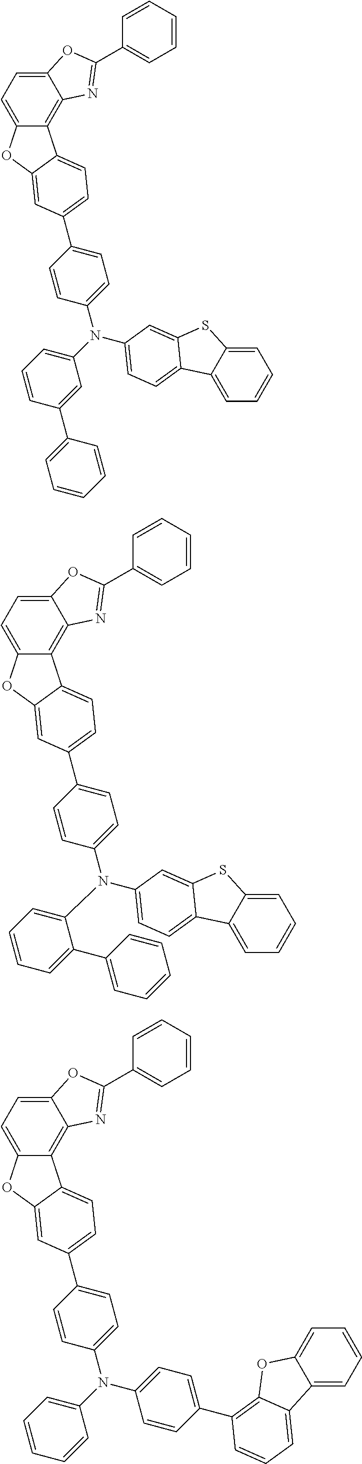

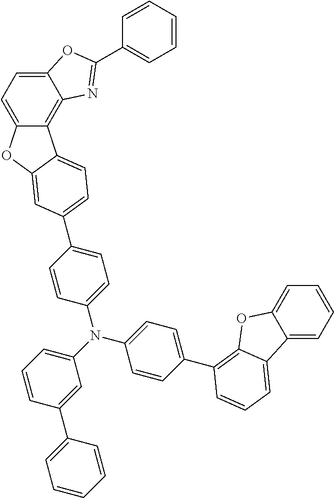 [SMILES: c1ccc(-c2cccc(N(c3ccc(-c4ccc5c(c4)oc4ccc6oc(-c7ccccc7)nc6c45)cc3)c3ccc(-c4cccc5c4oc4ccccc45)cc3)c2)cc1]

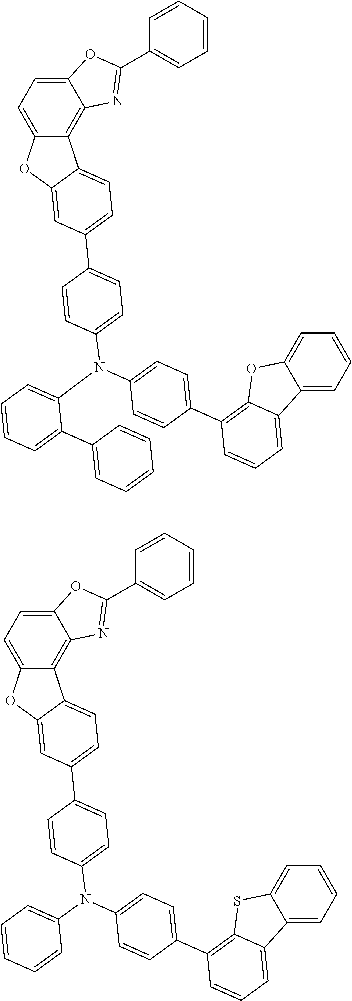 [SMILES: c1ccc(-c2nc3c(ccc4oc5cc(-c6ccc(N(c7ccc(-c8cccc9c8oc8ccccc89)cc7)c7ccccc7-c7ccccc7)cc6)ccc5c43)o2)cc1 ; c1ccc(-c2nc3c(ccc4oc5cc(-c6ccc(N(c7ccccc7)c7ccc(-c8cccc9c8sc8ccccc89)cc7)cc6)ccc5c43)o2)cc1]

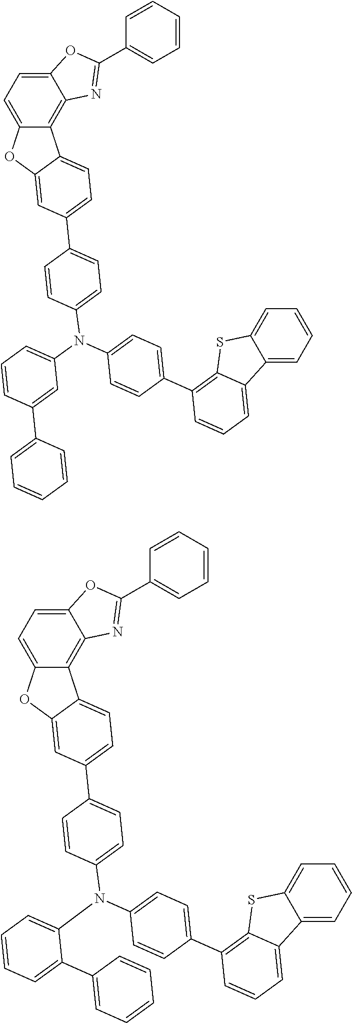 [SMILES: c1ccc(-c2cccc(N(c3ccc(-c4ccc5c(c4)oc4ccc6oc(-c7ccccc7)nc6c45)cc3)c3ccc(-c4cccc5c4sc4ccccc45)cc3)c2)cc1 ; c1ccc(-c2nc3c(ccc4oc5cc(-c6ccc(N(c7ccc(-c8cccc9c8sc8ccccc89)cc7)c7ccccc7-c7ccccc7)cc6)ccc5c43)o2)cc1]

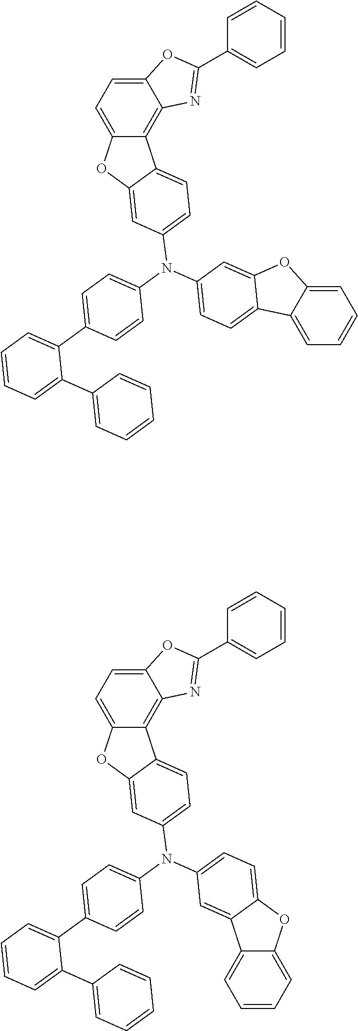 [SMILES: c1ccc(-c2nc3c(ccc4oc5cc(N(c6ccc(-c7ccccc7-c7ccccc7)cc6)c6ccc7c(c6)oc6ccccc67)ccc5c43)o2)cc1 ; c1ccc(-c2nc3c(ccc4oc5cc(N(c6ccc(-c7ccccc7-c7ccccc7)cc6)c6ccc7oc8ccccc8c7c6)ccc5c43)o2)cc1]

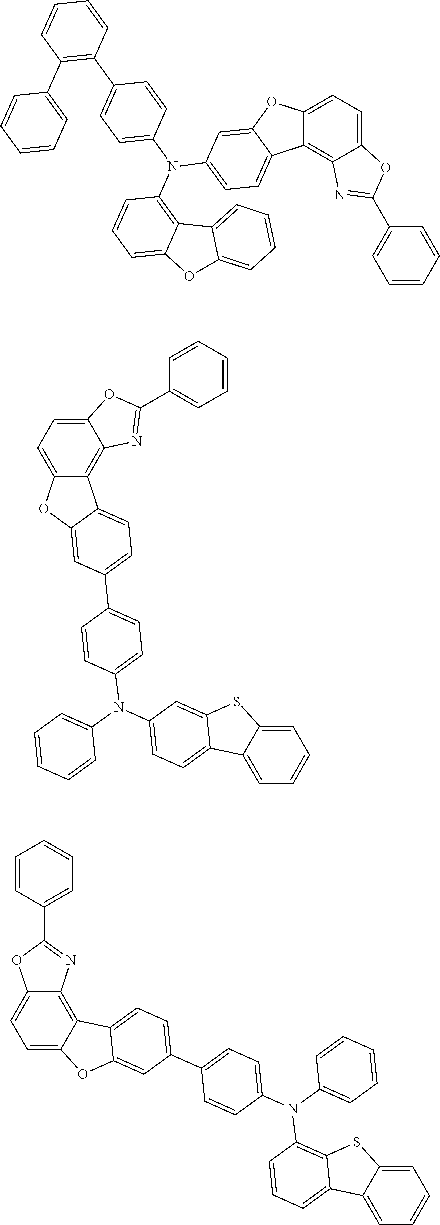 [SMILES: c1ccc(-c2nc3c(ccc4oc5cc(N(c6ccc(-c7ccccc7-c7ccccc7)cc6)c6cccc7oc8ccccc8c67)ccc5c43)o2)cc1 ; c1ccc(-c2nc3c(ccc4oc5cc(-c6ccc(N(c7ccccc7)c7ccc8c(c7)sc7ccccc78)cc6)ccc5c43)o2)cc1 ; c1ccc(-c2nc3c(ccc4oc5cc(-c6ccc(N(c7ccccc7)c7cccc8c7sc7ccccc78)cc6)ccc5c43)o2)cc1]

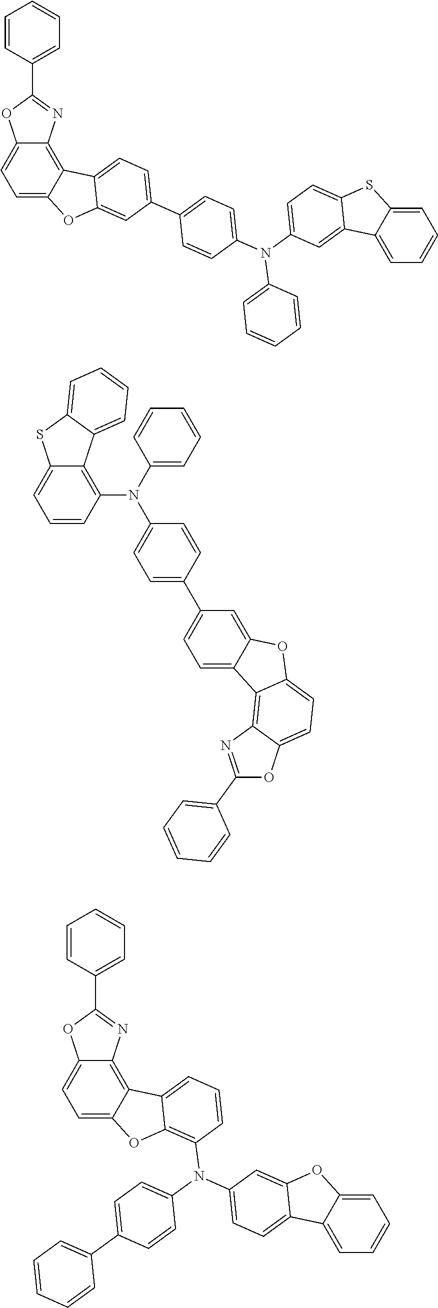 [SMILES: c1ccc(-c2nc3c(ccc4oc5cc(-c6ccc(N(c7ccccc7)c7ccc8sc9ccccc9c8c7)cc6)ccc5c43)o2)cc1 ; c1ccc(-c2nc3c(ccc4oc5cc(-c6ccc(N(c7ccccc7)c7cccc8sc9ccccc9c78)cc6)ccc5c43)o2)cc1 ; c1ccc(-c2ccc(N(c3ccc4c(c3)oc3ccccc34)c3cccc4c3oc3ccc5oc(-c6ccccc6)nc5c34)cc2)cc1]

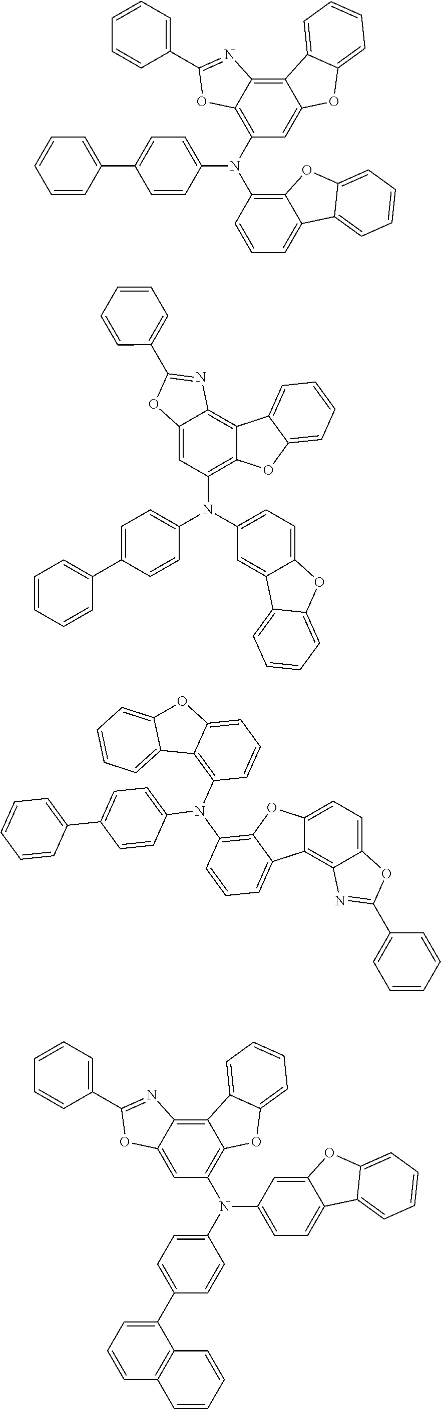 [SMILES: c1ccc(-c2ccc(N(c3cccc4c3oc3ccccc34)c3cc4oc5ccccc5c4c4nc(-c5ccccc5)oc34)cc2)cc1 ; c1ccc(-c2ccc(N(c3ccc4oc5ccccc5c4c3)c3cc4oc(-c5ccccc5)nc4c4c3oc3ccccc34)cc2)cc1 ; c1ccc(-c2ccc(N(c3cccc4c3oc3ccc5oc(-c6ccccc6)nc5c34)c3cccc4oc5ccccc5c34)cc2)cc1 ; c1ccc(-c2nc3c(cc(N(c4ccc(-c5cccc6ccccc56)cc4)c4ccc5c(c4)oc4ccccc45)c4oc5ccccc5c43)o2)cc1]

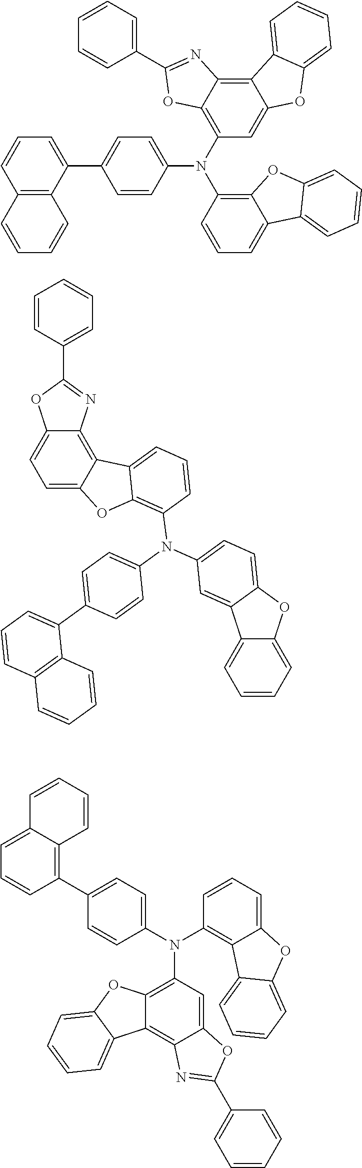

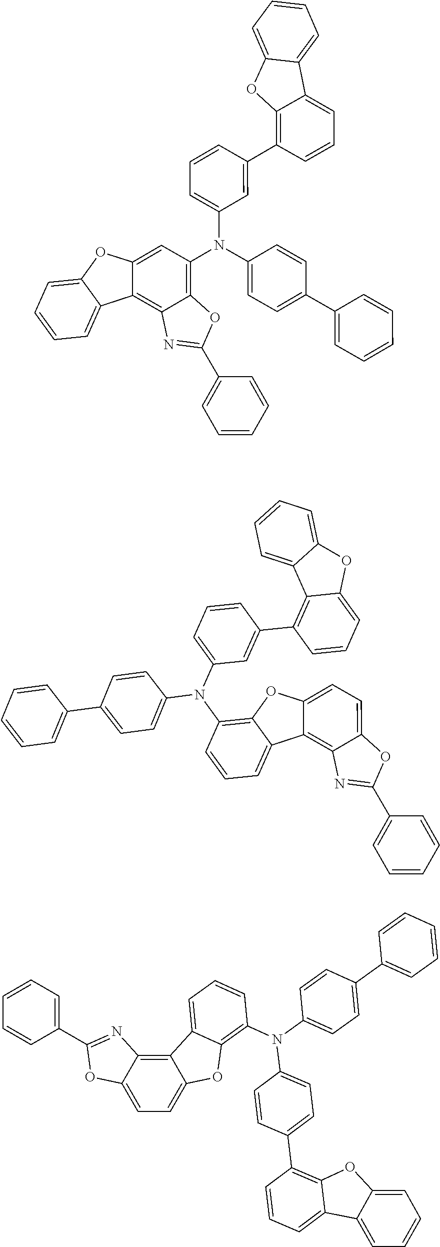

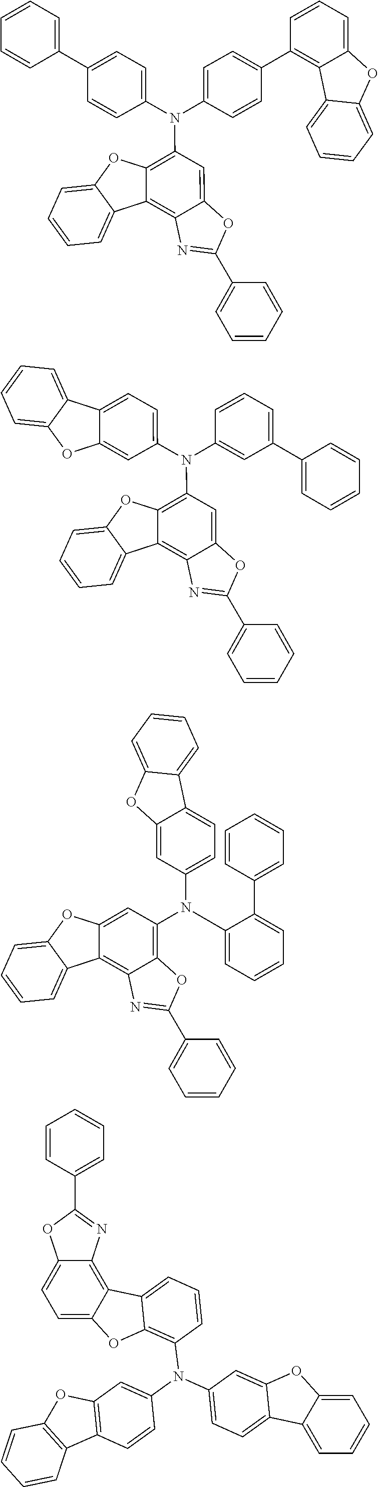 [SMILES: c1ccc(-c2ccc(N(c3ccc(-c4cccc5oc6ccccc6c45)cc3)c3cc4oc(-c5ccccc5)nc4c4c3oc3ccccc34)cc2)cc1 ; c1ccc(-c2cccc(N(c3ccc4c(c3)oc3ccccc34)c3cc4oc(-c5ccccc5)nc4c4c3oc3ccccc34)c2)cc1 ; c1ccc(-c2nc3c(o2)c(N(c2ccc4c(c2)oc2ccccc24)c2ccccc2-c2ccccc2)cc2oc4ccccc4c23)cc1 ; c1ccc(-c2nc3c(ccc4oc5c(N(c6ccc7c(c6)oc6ccccc67)c6ccc7c(c6)oc6ccccc67)cccc5c43)o2)cc1]

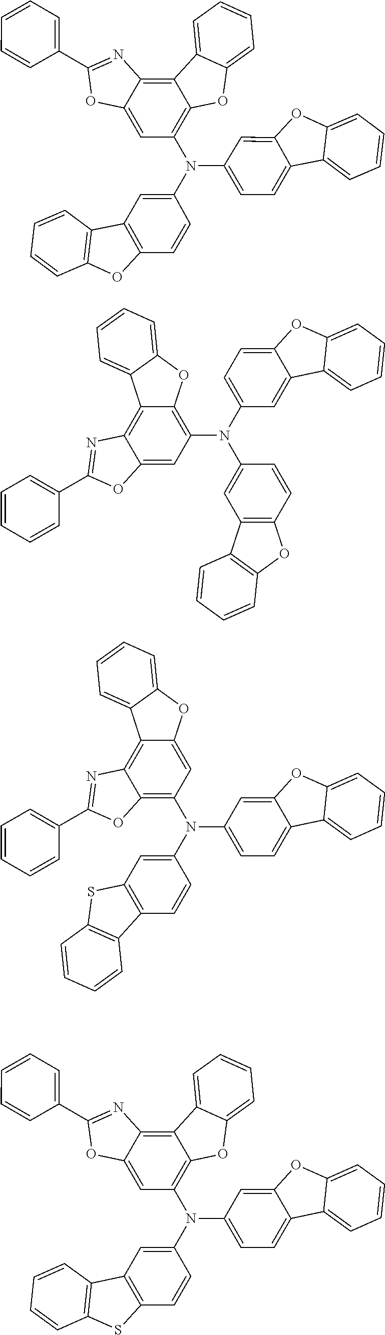

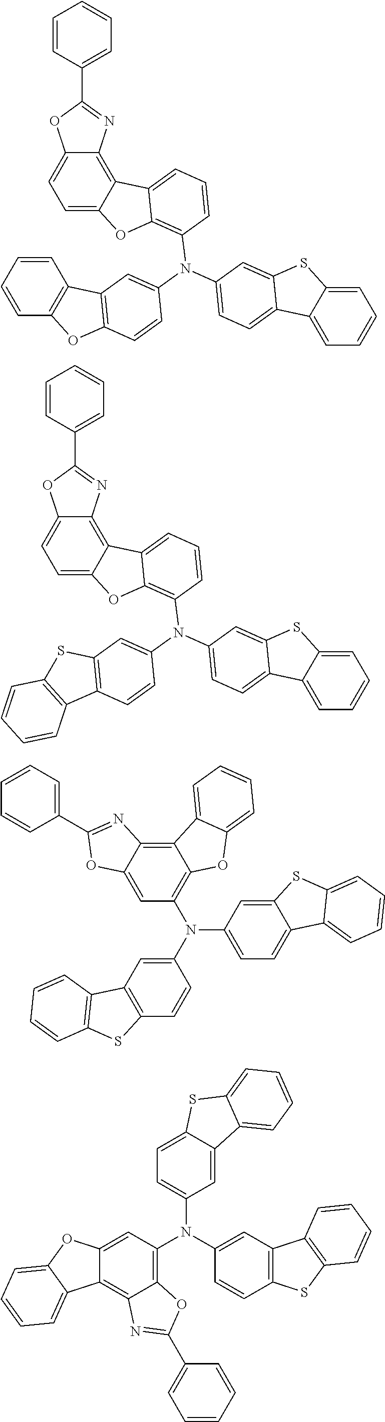

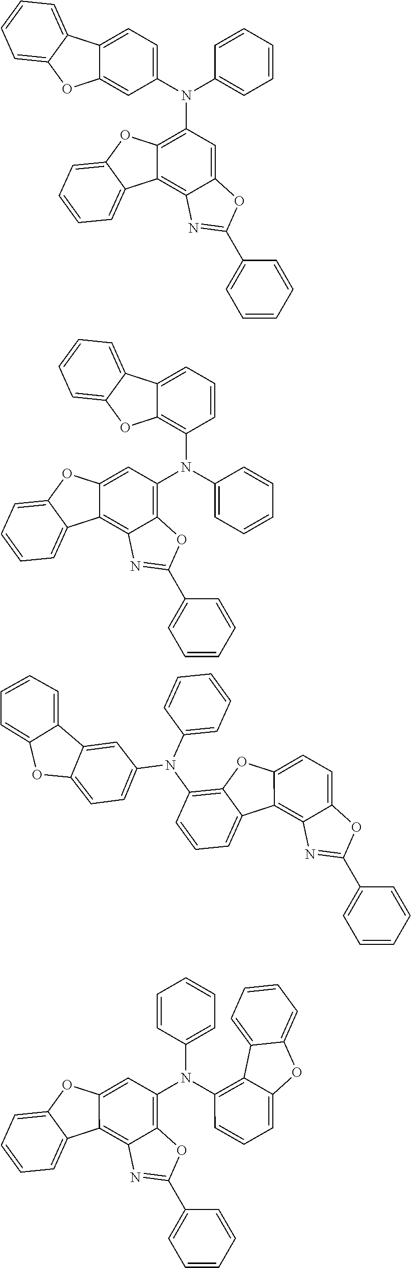

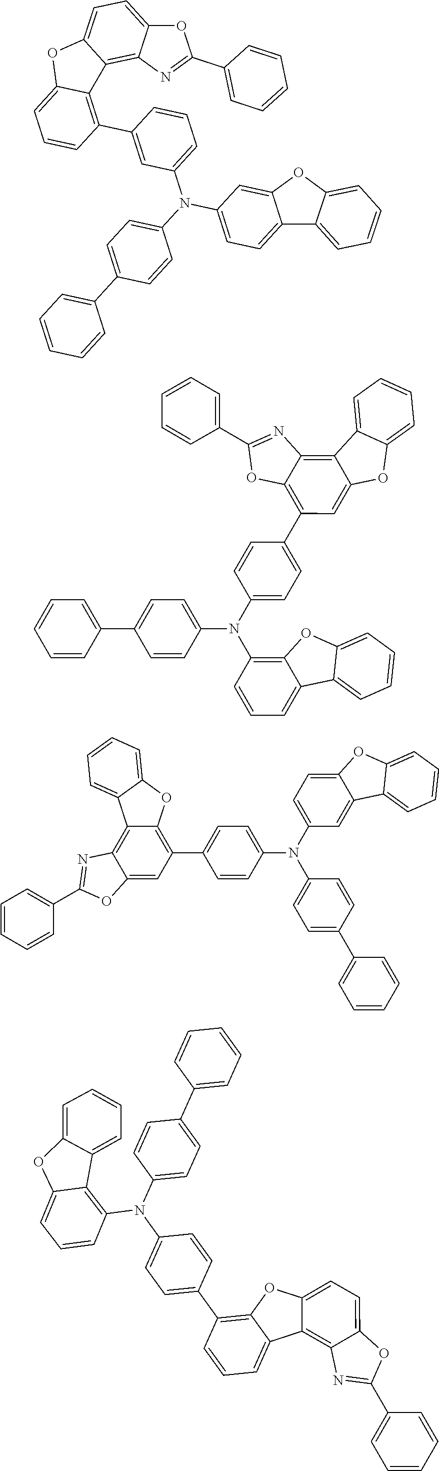 [SMILES: c1ccc(-c2ccc(N(c3cccc(-c4cccc5oc6ccc7oc(-c8ccccc8)nc7c6c45)c3)c3ccc4c(c3)oc3ccccc34)cc2)cc1 ; c1ccc(-c2ccc(N(c3ccc(-c4cc5oc6ccccc6c5c5nc(-c6ccccc6)oc45)cc3)c3cccc4c3oc3ccccc34)cc2)cc1 ; c1ccc(-c2ccc(N(c3ccc(-c4cc5oc(-c6ccccc6)nc5c5c4oc4ccccc45)cc3)c3ccc4oc5ccccc5c4c3)cc2)cc1 ; c1ccc(-c2ccc(N(c3ccc(-c4cccc5c4oc4ccc6oc(-c7ccccc7)nc6c45)cc3)c3cccc4oc5ccccc5c34)cc2)cc1]

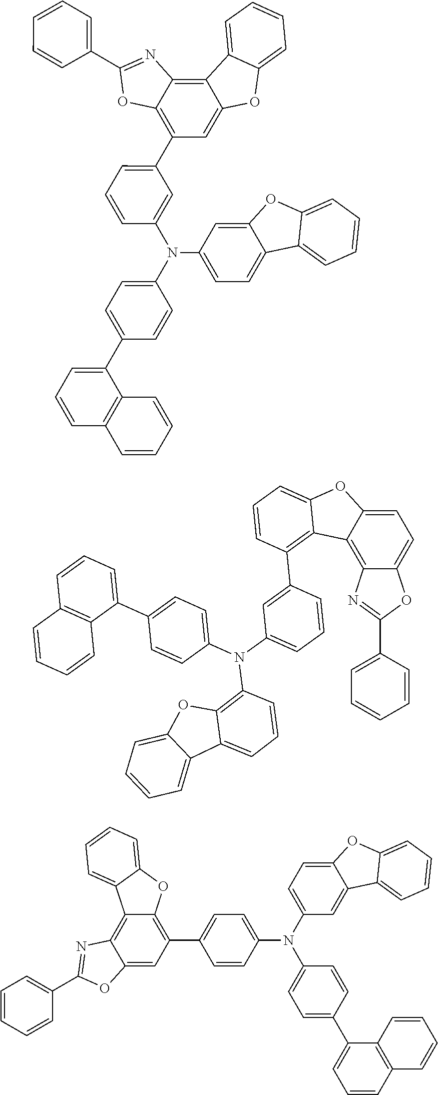

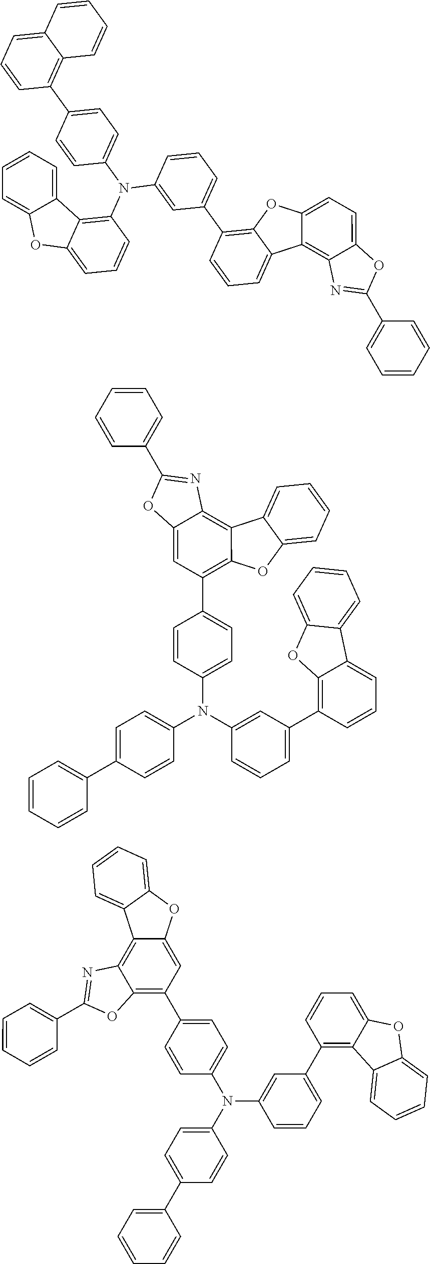 [SMILES: c1ccc(-c2nc3c(ccc4oc5c(-c6cccc(N(c7ccc(-c8cccc9ccccc89)cc7)c7cccc8oc9ccccc9c78)c6)cccc5c43)o2)cc1 ; c1ccc(-c2ccc(N(c3ccc(-c4cc5oc(-c6ccccc6)nc5c5c4oc4ccccc45)cc3)c3cccc(-c4cccc5c4oc4ccccc45)c3)cc2)cc1 ; c1ccc(-c2ccc(N(c3ccc(-c4cc5oc6ccccc6c5c5nc(-c6ccccc6)oc45)cc3)c3cccc(-c4cccc5oc6ccccc6c45)c3)cc2)cc1]

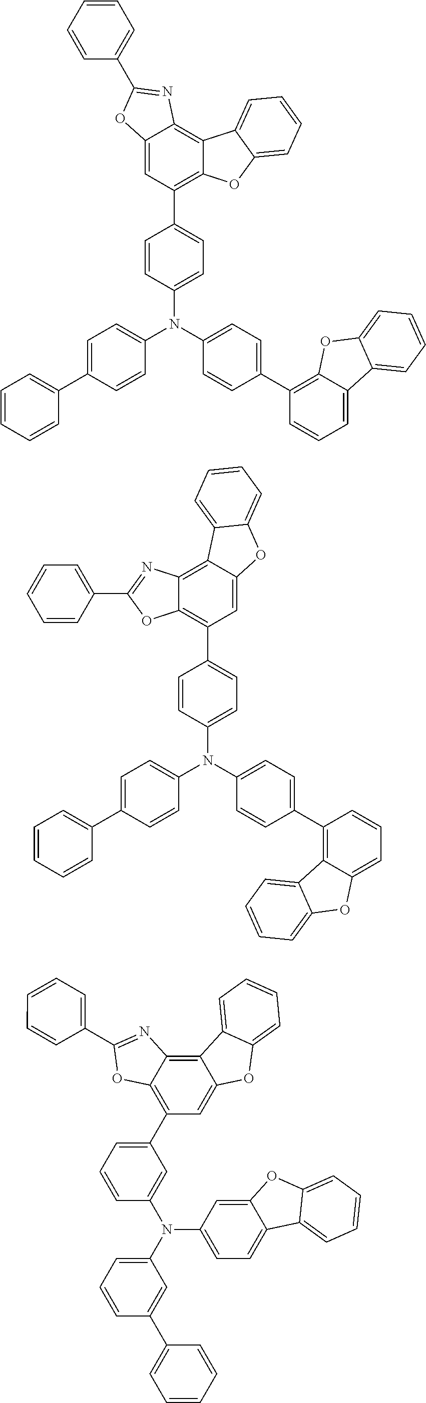

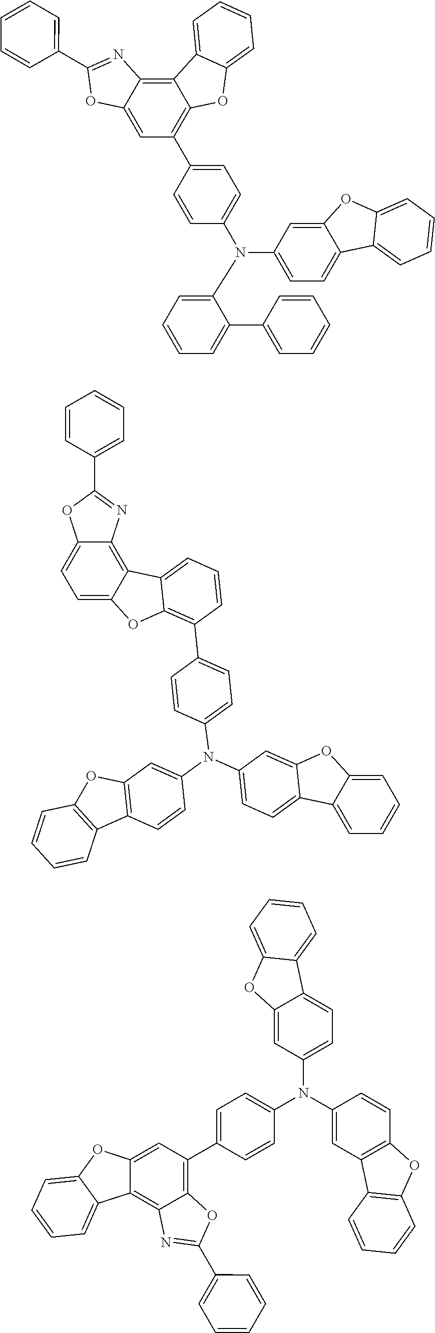 [SMILES: c1ccc(-c2nc3c(cc(-c4ccc(N(c5ccc6c(c5)oc5ccccc56)c5ccccc5-c5ccccc5)cc4)c4oc5ccccc5c43)o2)cc1 ; c1ccc(-c2nc3c(ccc4oc5c(-c6ccc(N(c7ccc8c(c7)oc7ccccc78)c7ccc8c(c7)oc7ccccc78)cc6)cccc5c43)o2)cc1 ; c1ccc(-c2nc3c(o2)c(-c2ccc(N(c4ccc5c(c4)oc4ccccc45)c4ccc5oc6ccccc6c5c4)cc2)cc2oc4ccccc4c23)cc1]

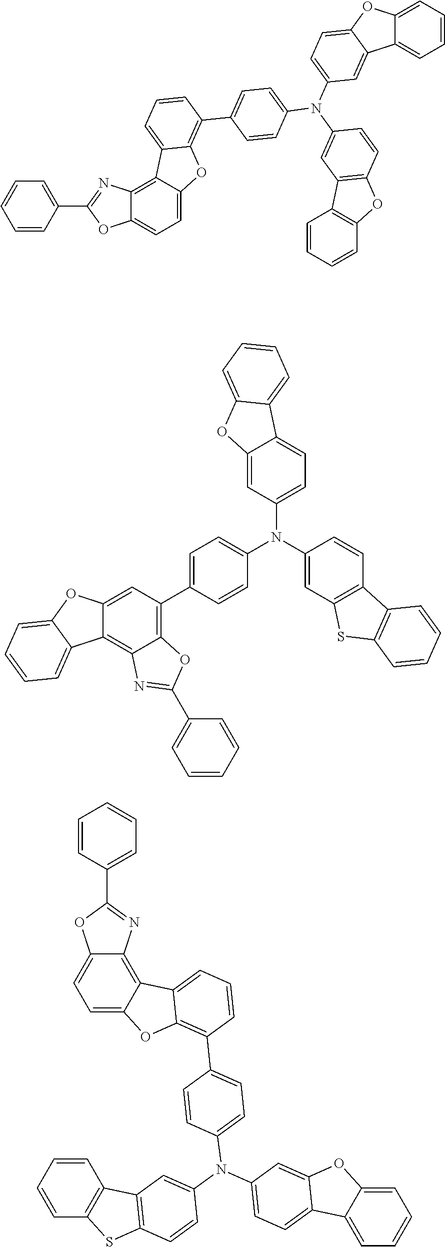 [SMILES: c1ccc(-c2nc3c(ccc4oc5c(-c6ccc(N(c7ccc8oc9ccccc9c8c7)c7ccc8oc9ccccc9c8c7)cc6)cccc5c43)o2)cc1 ; c1ccc(-c2nc3c(o2)c(-c2ccc(N(c4ccc5c(c4)oc4ccccc45)c4ccc5c(c4)sc4ccccc45)cc2)cc2oc4ccccc4c23)cc1 ; c1ccc(-c2nc3c(ccc4oc5c(-c6ccc(N(c7ccc8c(c7)oc7ccccc78)c7ccc8sc9ccccc9c8c7)cc6)cccc5c43)o2)cc1]

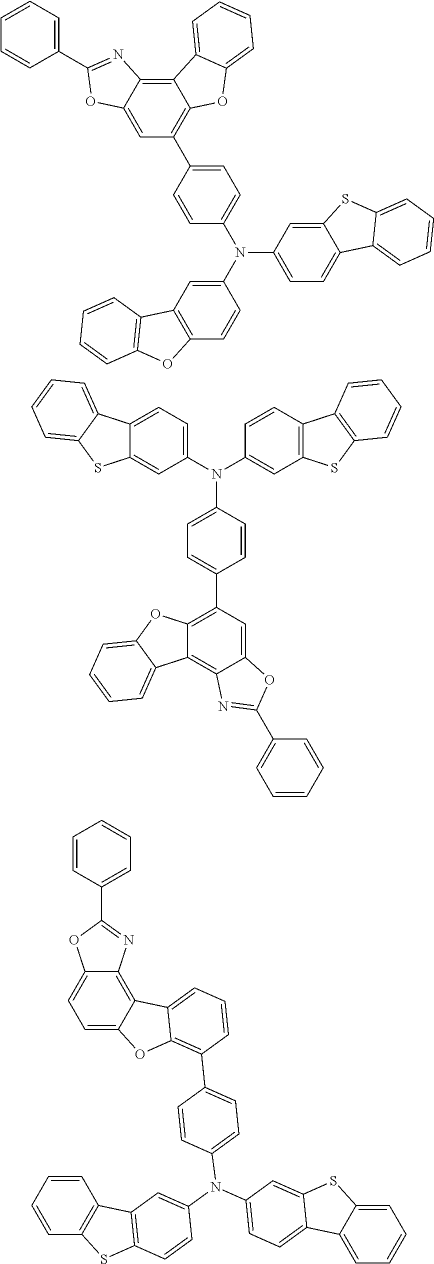

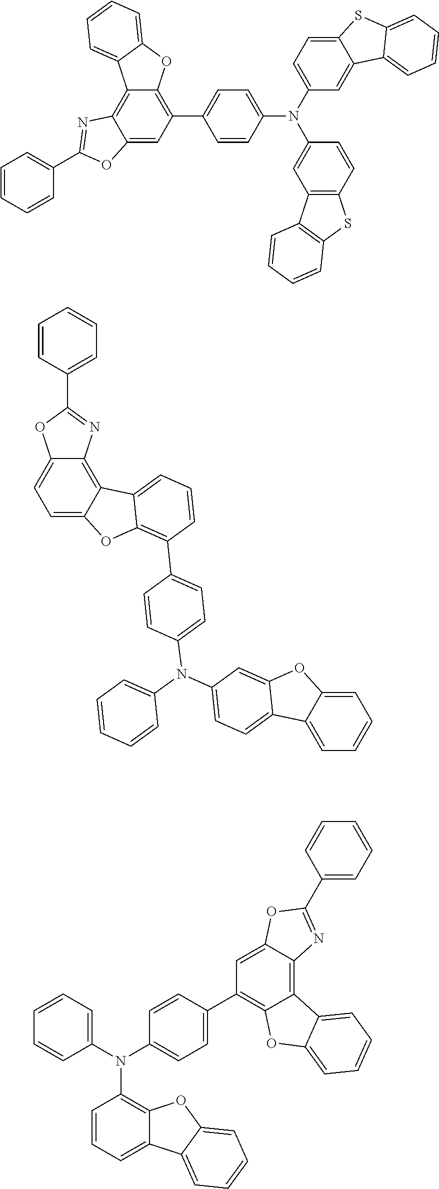 [SMILES: c1ccc(-c2nc3c(cc(-c4ccc(N(c5ccc6sc7ccccc7c6c5)c5ccc6sc7ccccc7c6c5)cc4)c4oc5ccccc5c43)o2)cc1 ; c1ccc(-c2nc3c(ccc4oc5c(-c6ccc(N(c7ccccc7)c7ccc8c(c7)oc7ccccc78)cc6)cccc5c43)o2)cc1 ; c1ccc(-c2nc3c(cc(-c4ccc(N(c5ccccc5)c5cccc6c5oc5ccccc56)cc4)c4oc5ccccc5c43)o2)cc1]

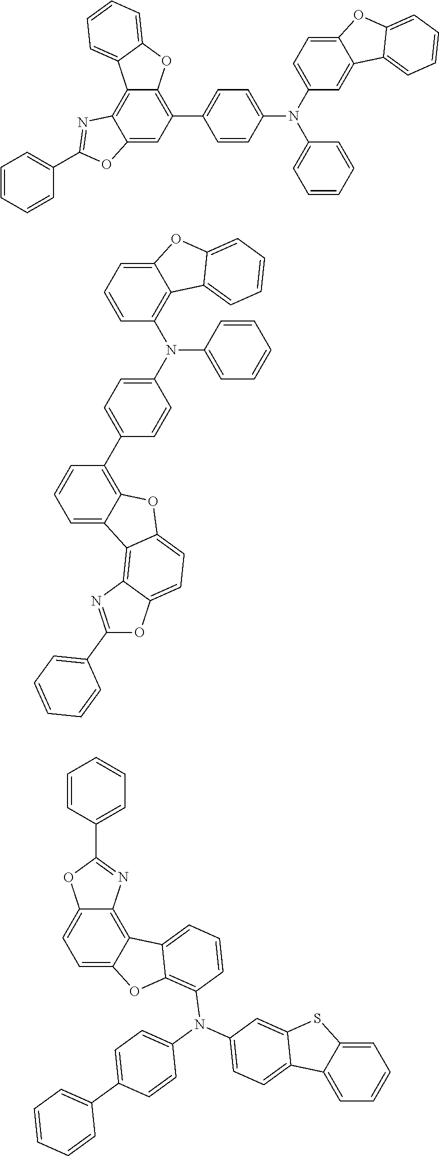

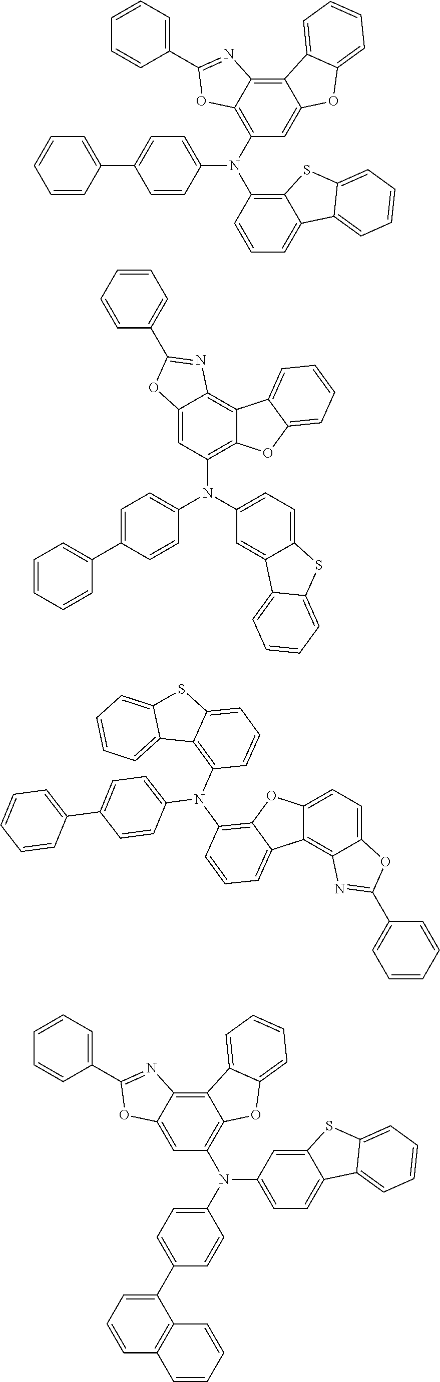 [SMILES: c1ccc(-c2ccc(N(c3cc4oc5ccccc5c4c4nc(-c5ccccc5)oc34)c3cccc4c3sc3ccccc34)cc2)cc1 ; c1ccc(-c2ccc(N(c3ccc4sc5ccccc5c4c3)c3cc4oc(-c5ccccc5)nc4c4c3oc3ccccc34)cc2)cc1 ; c1ccc(-c2ccc(N(c3cccc4c3oc3ccc5oc(-c6ccccc6)nc5c34)c3cccc4sc5ccccc5c34)cc2)cc1 ; c1ccc(-c2nc3c(cc(N(c4ccc(-c5cccc6ccccc56)cc4)c4ccc5c(c4)sc4ccccc45)c4oc5ccccc5c43)o2)cc1]

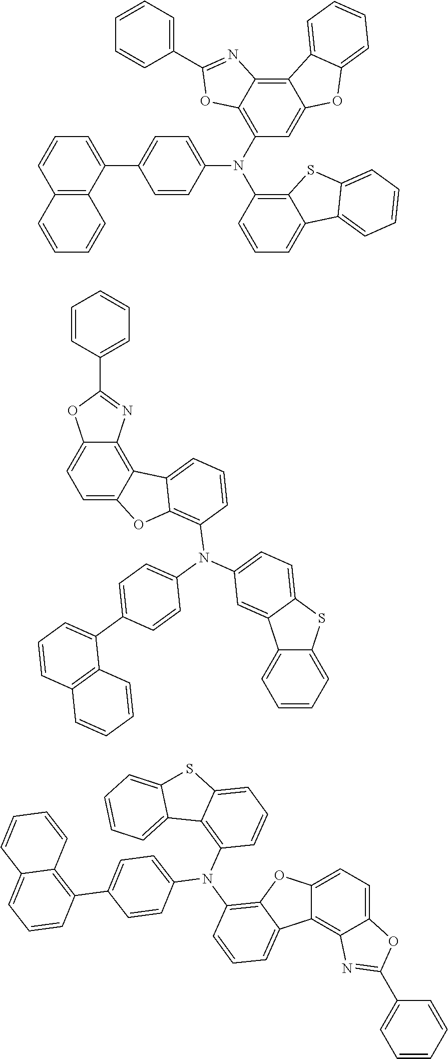

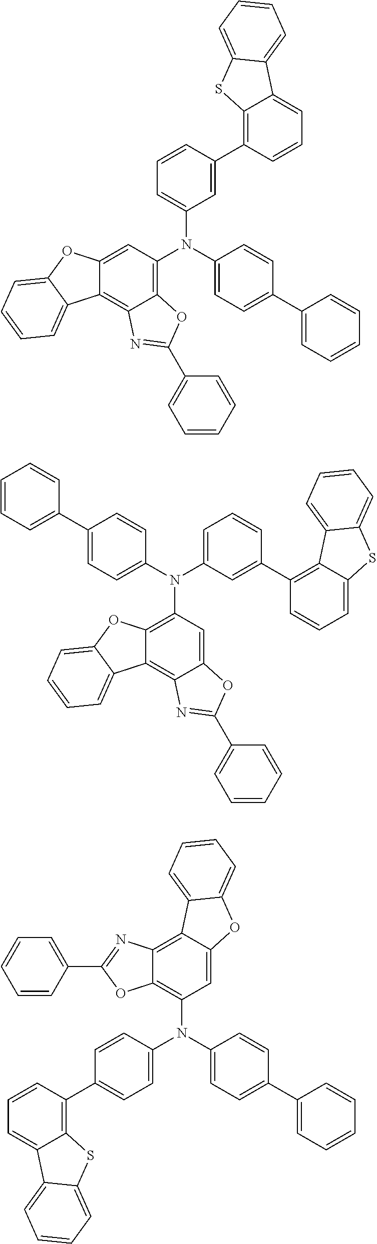

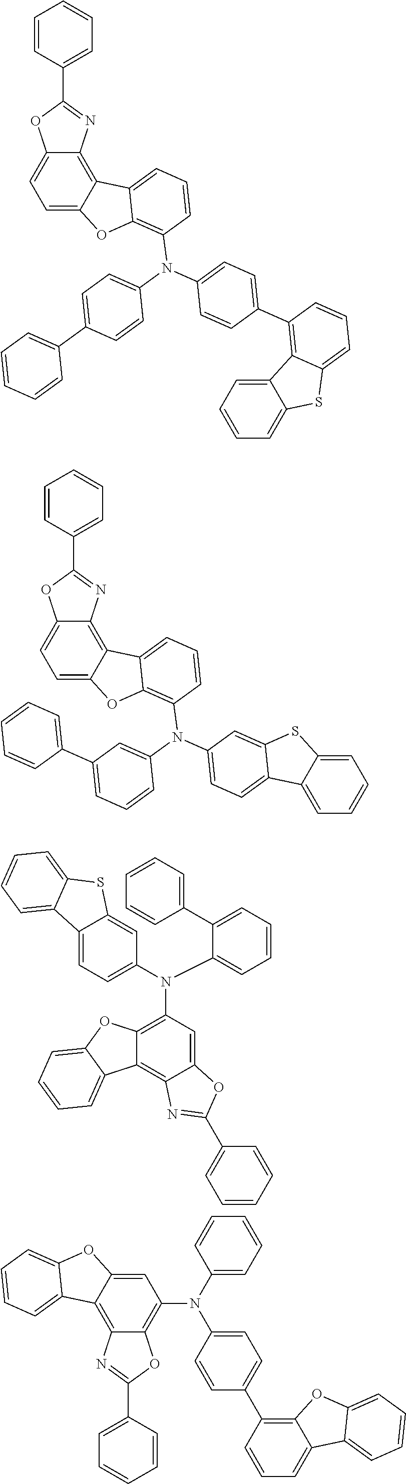

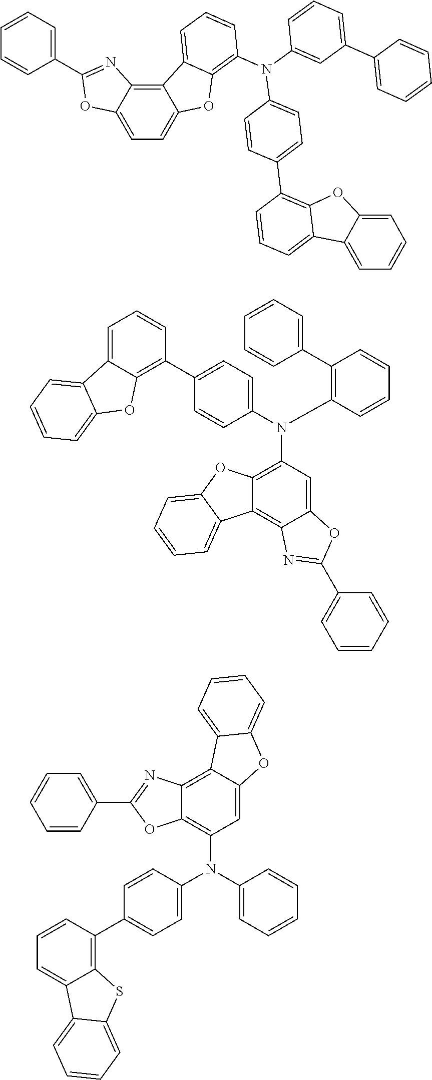 [SMILES: c1ccc(-c2cccc(N(c3ccc(-c4cccc5c4oc4ccccc45)cc3)c3cccc4c3oc3ccc5oc(-c6ccccc6)nc5c34)c2)cc1 ; c1ccc(-c2nc3c(cc(N(c4ccc(-c5cccc6c5oc5ccccc56)cc4)c4ccccc4-c4ccccc4)c4oc5ccccc5c43)o2)cc1 ; c1ccc(-c2nc3c(o2)c(N(c2ccccc2)c2ccc(-c4cccc5c4sc4ccccc45)cc2)cc2oc4ccccc4c23)cc1]

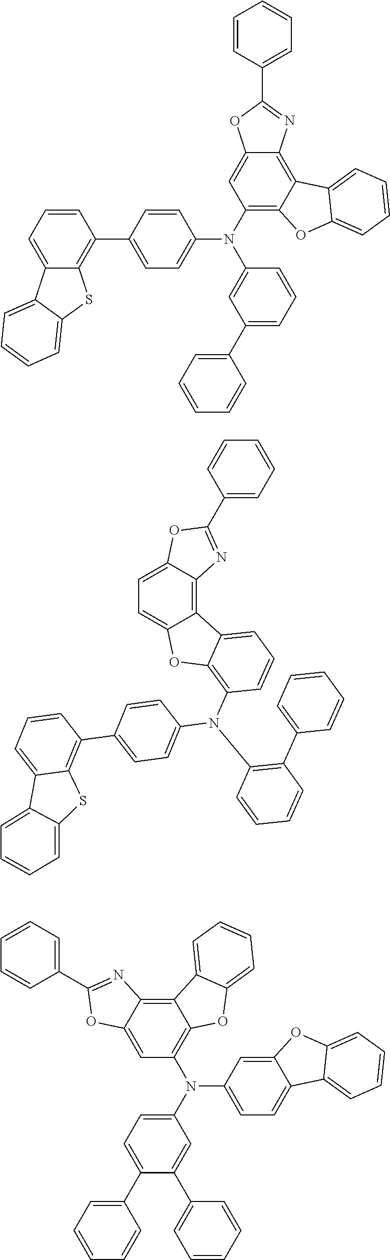

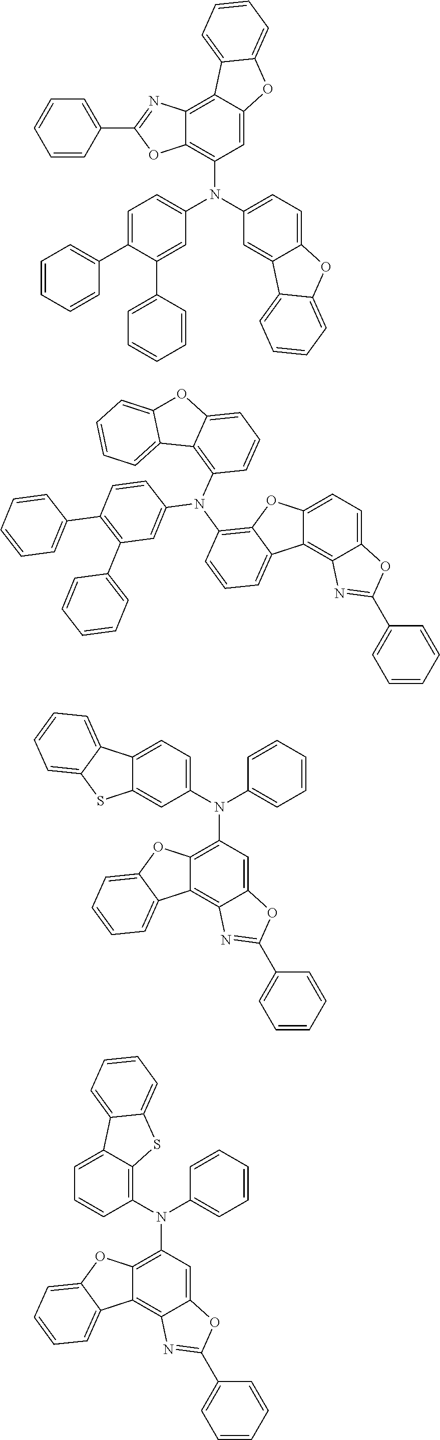

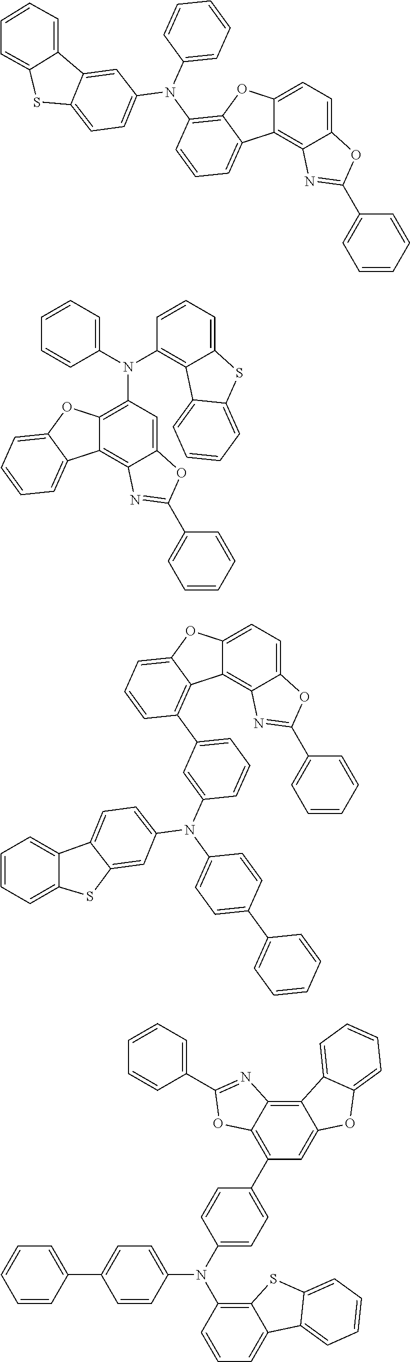

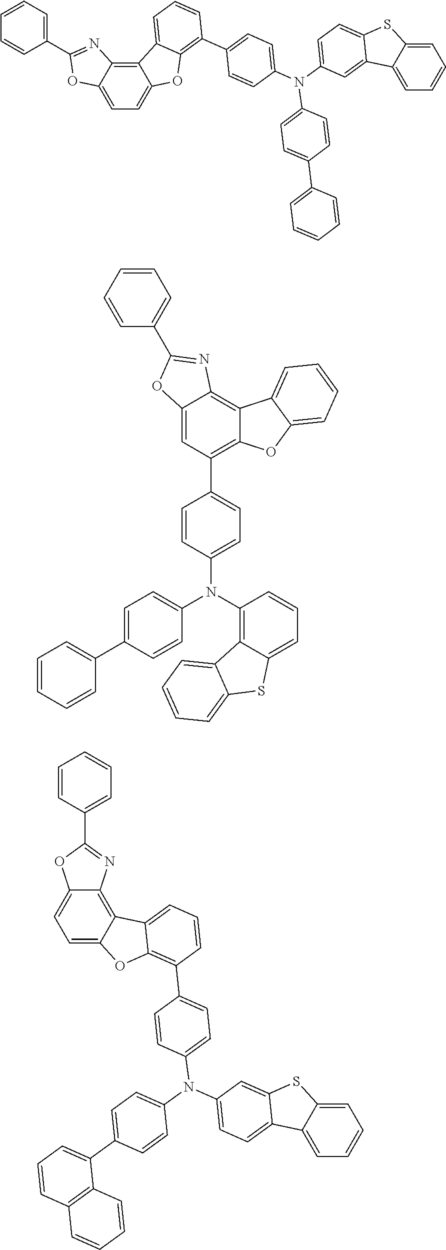 [SMILES: c1ccc(-c2ccc(N(c3ccc(-c4cccc5c4oc4ccc6oc(-c7ccccc7)nc6c45)cc3)c3ccc4sc5ccccc5c4c3)cc2)cc1 ; c1ccc(-c2ccc(N(c3ccc(-c4cc5oc(-c6ccccc6)nc5c5c4oc4ccccc45)cc3)c3cccc4sc5ccccc5c34)cc2)cc1 ; c1ccc(-c2nc3c(ccc4oc5c(-c6ccc(N(c7ccc(-c8cccc9ccccc89)cc7)c7ccc8c(c7)sc7ccccc78)cc6)cccc5c43)o2)cc1]

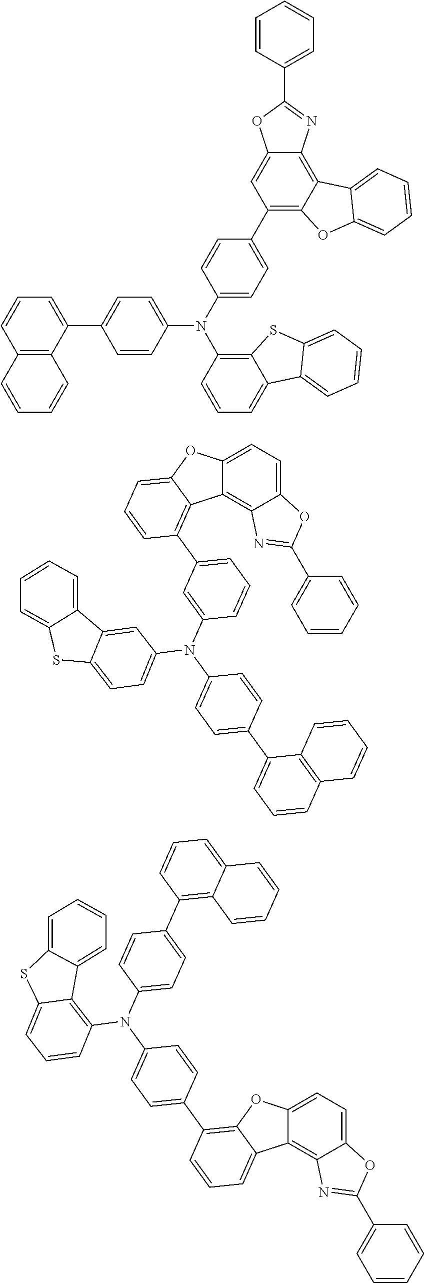

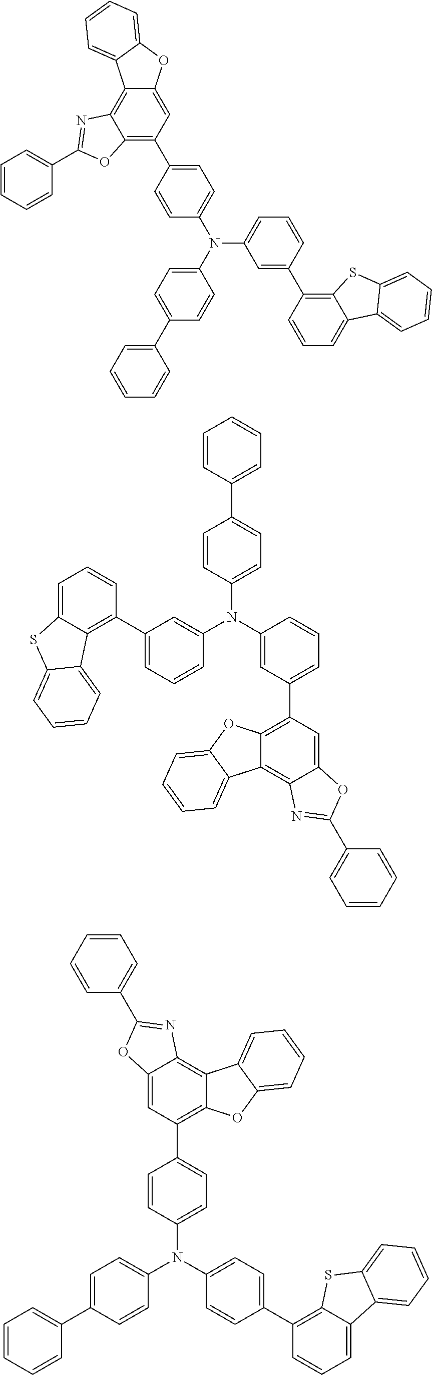 [SMILES: c1ccc(-c2ccc(N(c3ccc(-c4cc5oc6ccccc6c5c5nc(-c6ccccc6)oc45)cc3)c3cccc(-c4cccc5c4sc4ccccc45)c3)cc2)cc1 ; c1ccc(-c2ccc(N(c3cccc(-c4cc5oc(-c6ccccc6)nc5c5c4oc4ccccc45)c3)c3cccc(-c4cccc5sc6ccccc6c45)c3)cc2)cc1 ; c1ccc(-c2ccc(N(c3ccc(-c4cc5oc(-c6ccccc6)nc5c5c4oc4ccccc45)cc3)c3ccc(-c4cccc5c4sc4ccccc45)cc3)cc2)cc1]

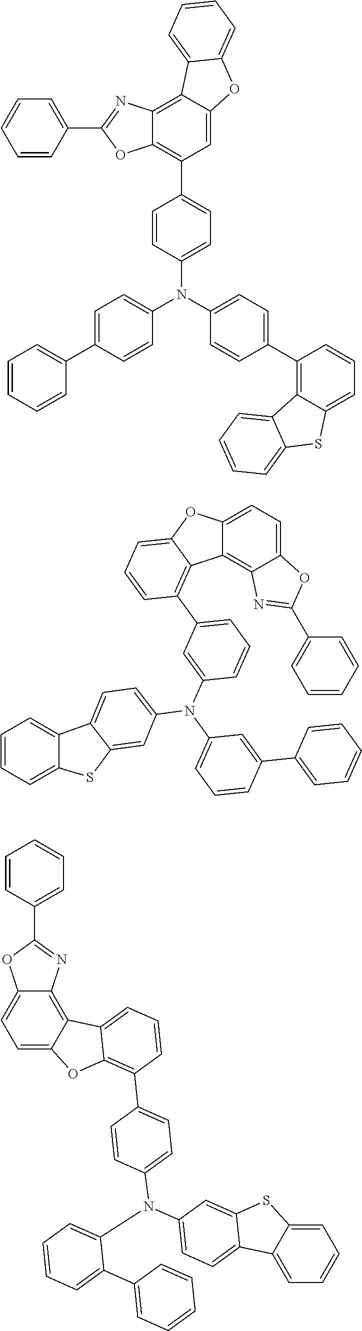

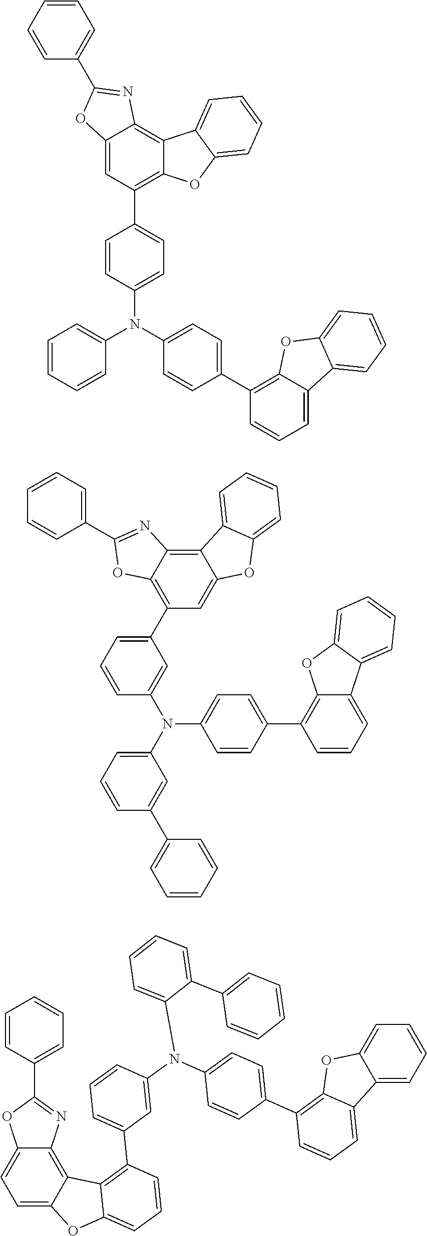

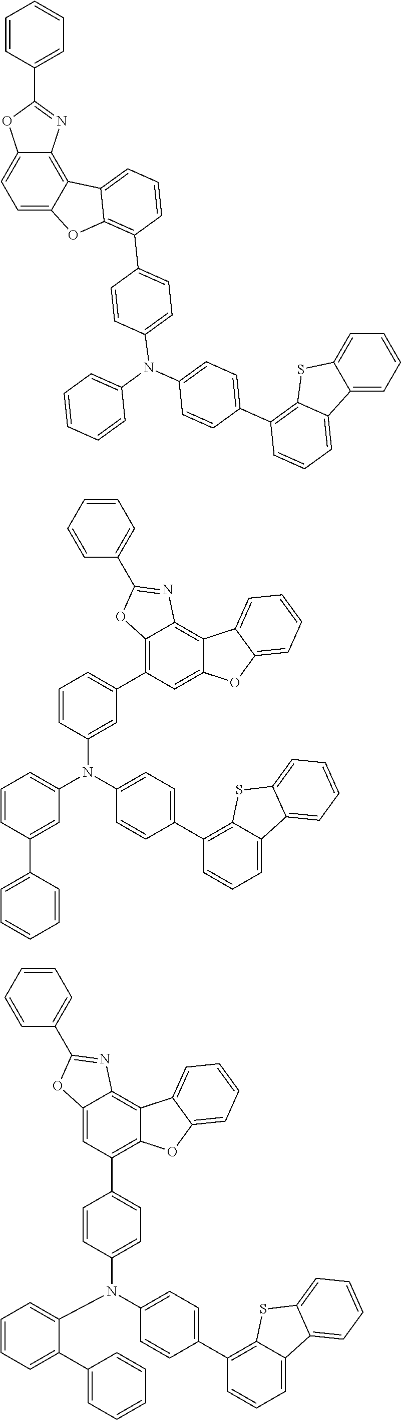

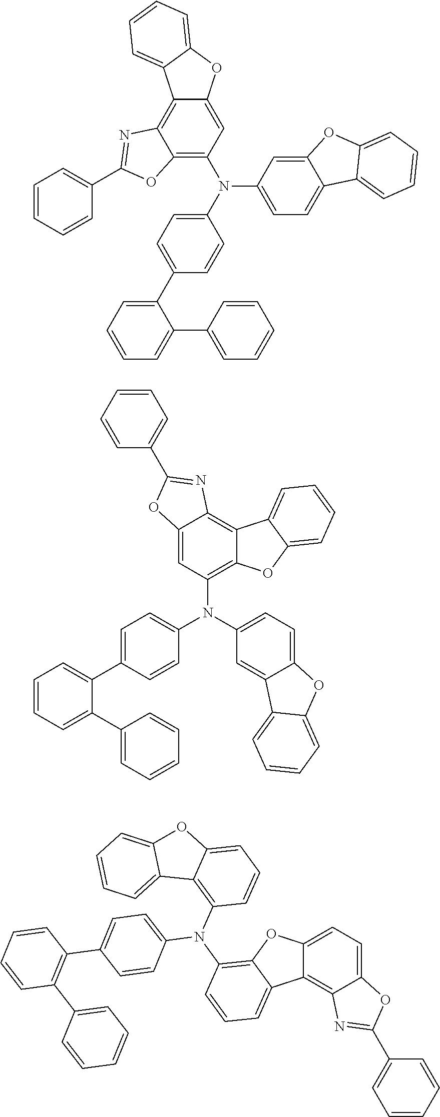

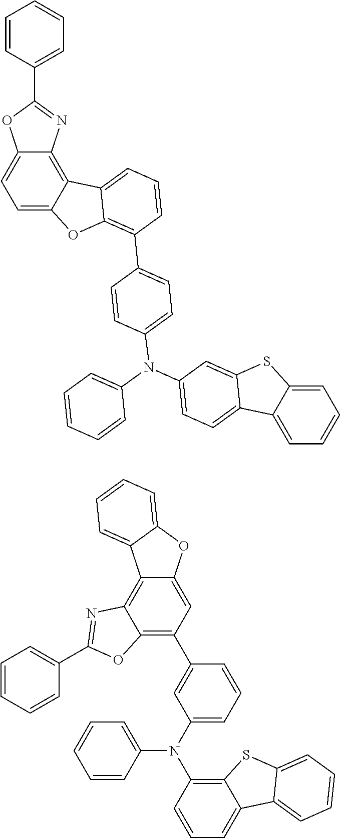 [SMILES: c1ccc(-c2nc3c(ccc4oc5c(-c6ccc(N(c7ccccc7)c7ccc8c(c7)sc7ccccc78)cc6)cccc5c43)o2)cc1 ; c1ccc(-c2nc3c(o2)c(-c2cccc(N(c4ccccc4)c4cccc5c4sc4ccccc45)c2)cc2oc4ccccc4c23)cc1]

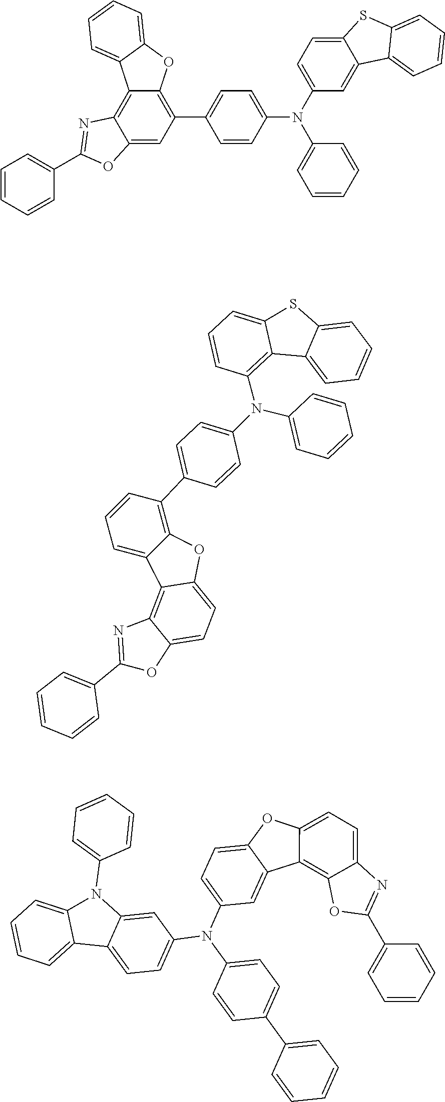 [SMILES: c1ccc(-c2nc3c(cc(-c4ccc(N(c5ccccc5)c5ccc6sc7ccccc7c6c5)cc4)c4oc5ccccc5c43)o2)cc1 ; c1ccc(-c2nc3c(ccc4oc5c(-c6ccc(N(c7ccccc7)c7cccc8sc9ccccc9c78)cc6)cccc5c43)o2)cc1 ; c1ccc(-c2ccc(N(c3ccc4oc5ccc6nc(-c7ccccc7)oc6c5c4c3)c3ccc4c5ccccc5n(-c5ccccc5)c4c3)cc2)cc1]

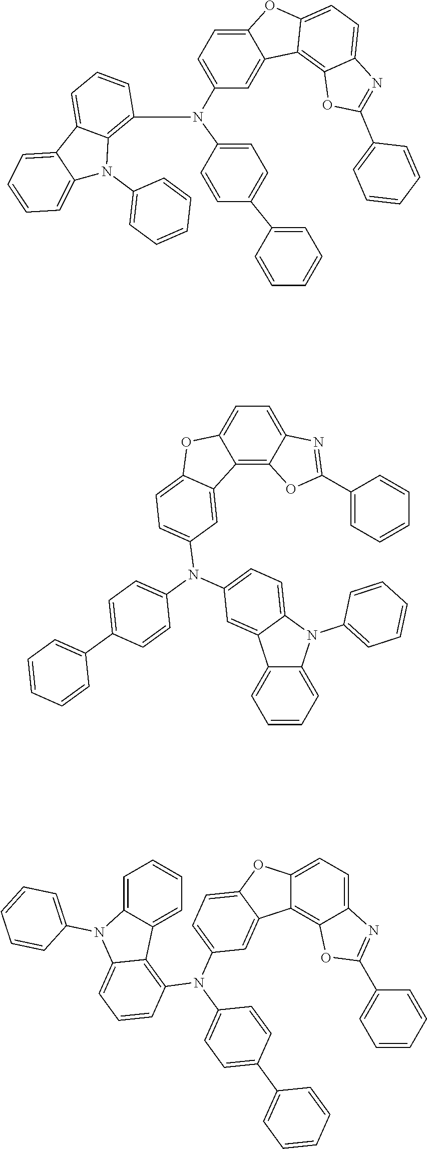 [SMILES: c1ccc(-c2ccc(N(c3ccc4oc5ccc6nc(-c7ccccc7)oc6c5c4c3)c3cccc4c5ccccc5n(-c5ccccc5)c34)cc2)cc1 ; c1ccc(-c2ccc(N(c3ccc4oc5ccc6nc(-c7ccccc7)oc6c5c4c3)c3ccc4c(c3)c3ccccc3n4-c3ccccc3)cc2)cc1 ; c1ccc(-c2ccc(N(c3ccc4oc5ccc6nc(-c7ccccc7)oc6c5c4c3)c3cccc4c3c3ccccc3n4-c3ccccc3)cc2)cc1]

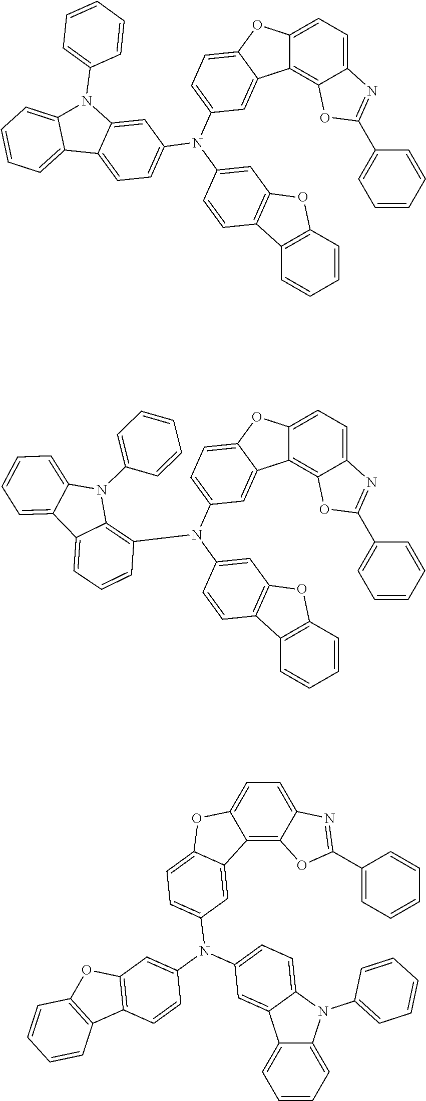 [SMILES: c1ccc(-c2nc3ccc4oc5ccc(N(c6ccc7c(c6)oc6ccccc67)c6ccc7c8ccccc8n(-c8ccccc8)c7c6)cc5c4c3o2)cc1 ; c1ccc(-c2nc3ccc4oc5ccc(N(c6ccc7c(c6)oc6ccccc67)c6cccc7c8ccccc8n(-c8ccccc8)c67)cc5c4c3o2)cc1 ; c1ccc(-c2nc3ccc4oc5ccc(N(c6ccc7c(c6)oc6ccccc67)c6ccc7c(c6)c6ccccc6n7-c6ccccc6)cc5c4c3o2)cc1]

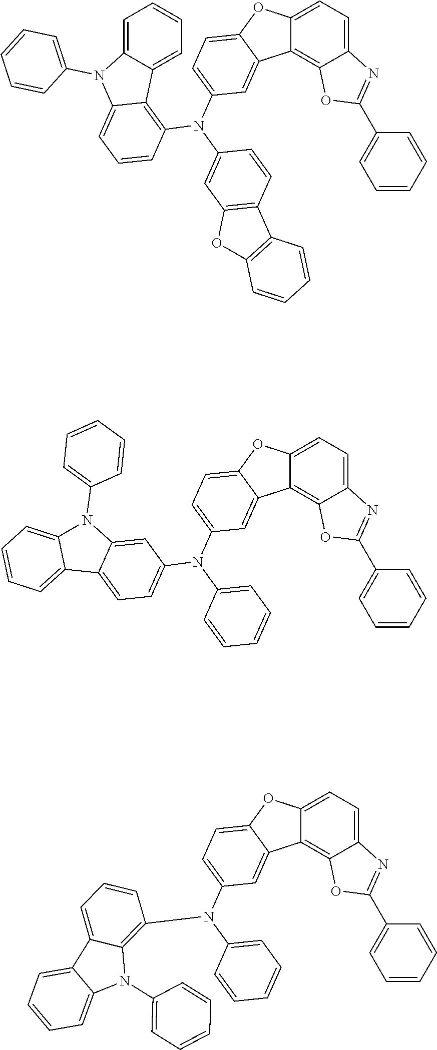 [SMILES: c1ccc(-c2nc3ccc4oc5ccc(N(c6ccc7c(c6)oc6ccccc67)c6cccc7c6c6ccccc6n7-c6ccccc6)cc5c4c3o2)cc1 ; c1ccc(-c2nc3ccc4oc5ccc(N(c6ccccc6)c6ccc7c8ccccc8n(-c8ccccc8)c7c6)cc5c4c3o2)cc1 ; c1ccc(-c2nc3ccc4oc5ccc(N(c6ccccc6)c6cccc7c8ccccc8n(-c8ccccc8)c67)cc5c4c3o2)cc1]

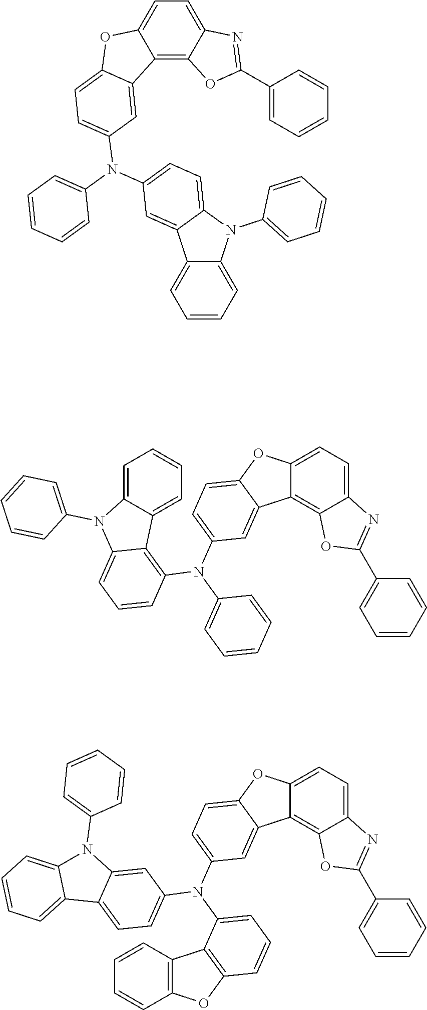 [SMILES: c1ccc(-c2nc3ccc4oc5ccc(N(c6ccccc6)c6ccc7c(c6)c6ccccc6n7-c6ccccc6)cc5c4c3o2)cc1 ; c1ccc(-c2nc3ccc4oc5ccc(N(c6ccccc6)c6cccc7c6c6ccccc6n7-c6ccccc6)cc5c4c3o2)cc1 ; c1ccc(-c2nc3ccc4oc5ccc(N(c6ccc7c8ccccc8n(-c8ccccc8)c7c6)c6cccc7oc8ccccc8c67)cc5c4c3o2)cc1]

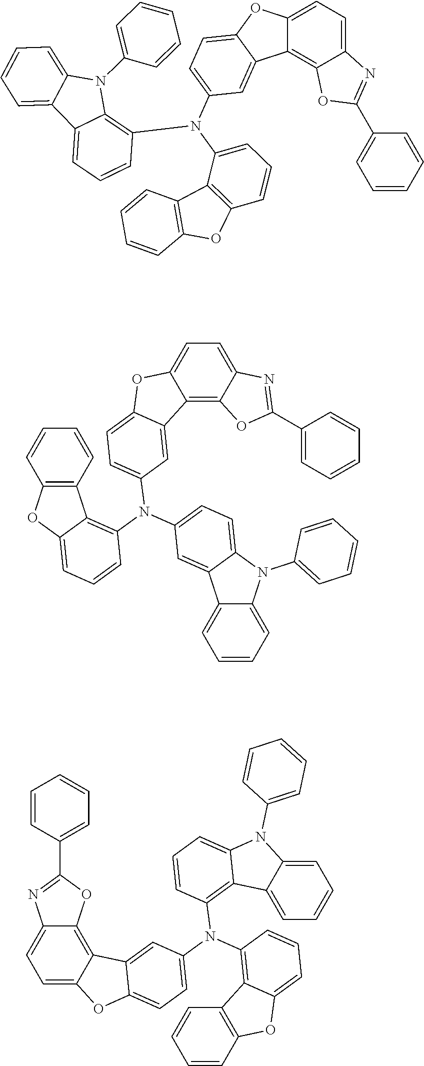 [SMILES: c1ccc(-c2nc3ccc4oc5ccc(N(c6cccc7oc8ccccc8c67)c6cccc7c8ccccc8n(-c8ccccc8)c67)cc5c4c3o2)cc1 ; c1ccc(-c2nc3ccc4oc5ccc(N(c6ccc7c(c6)c6ccccc6n7-c6ccccc6)c6cccc7oc8ccccc8c67)cc5c4c3o2)cc1 ; c1ccc(-c2nc3ccc4oc5ccc(N(c6cccc7oc8ccccc8c67)c6cccc7c6c6ccccc6n7-c6ccccc6)cc5c4c3o2)cc1]

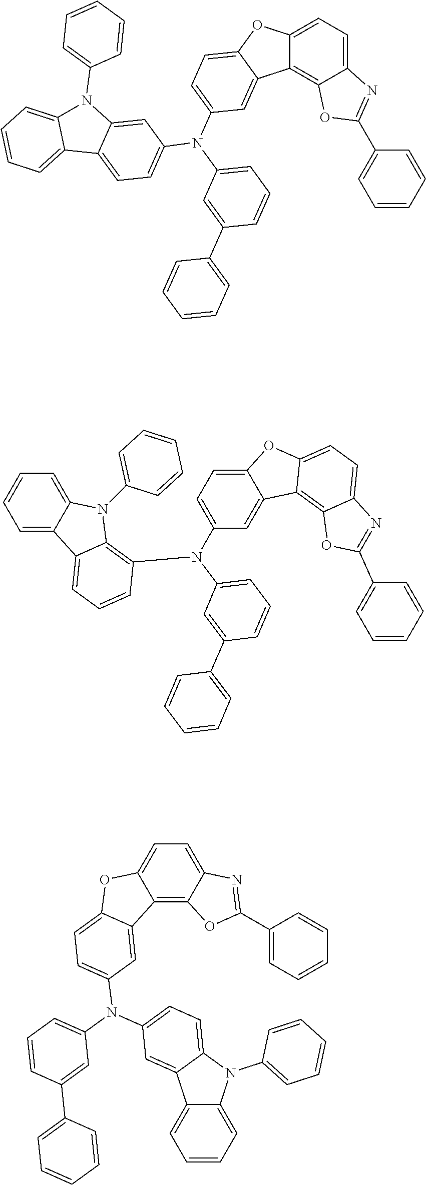 [SMILES: c1ccc(-c2cccc(N(c3ccc4oc5ccc6nc(-c7ccccc7)oc6c5c4c3)c3ccc4c5ccccc5n(-c5ccccc5)c4c3)c2)cc1 ; c1ccc(-c2cccc(N(c3ccc4oc5ccc6nc(-c7ccccc7)oc6c5c4c3)c3cccc4c5ccccc5n(-c5ccccc5)c34)c2)cc1 ; c1ccc(-c2cccc(N(c3ccc4oc5ccc6nc(-c7ccccc7)oc6c5c4c3)c3ccc4c(c3)c3ccccc3n4-c3ccccc3)c2)cc1]

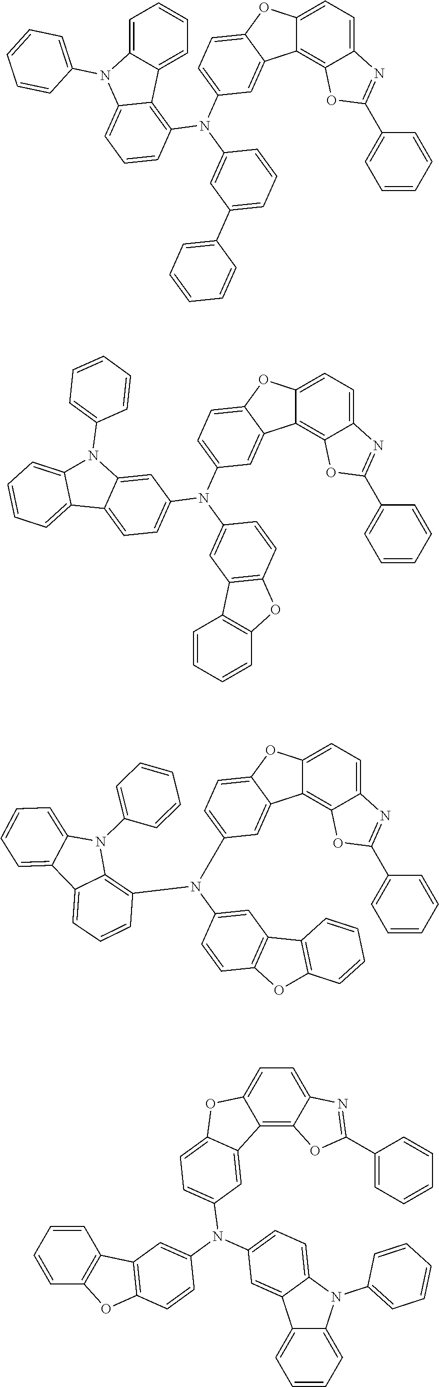 [SMILES: c1ccc(-c2cccc(N(c3ccc4oc5ccc6nc(-c7ccccc7)oc6c5c4c3)c3cccc4c3c3ccccc3n4-c3ccccc3)c2)cc1 ; c1ccc(-c2nc3ccc4oc5ccc(N(c6ccc7oc8ccccc8c7c6)c6ccc7c8ccccc8n(-c8ccccc8)c7c6)cc5c4c3o2)cc1 ; c1ccc(-c2nc3ccc4oc5ccc(N(c6ccc7oc8ccccc8c7c6)c6cccc7c8ccccc8n(-c8ccccc8)c67)cc5c4c3o2)cc1 ; c1ccc(-c2nc3ccc4oc5ccc(N(c6ccc7oc8ccccc8c7c6)c6ccc7c(c6)c6ccccc6n7-c6ccccc6)cc5c4c3o2)cc1]

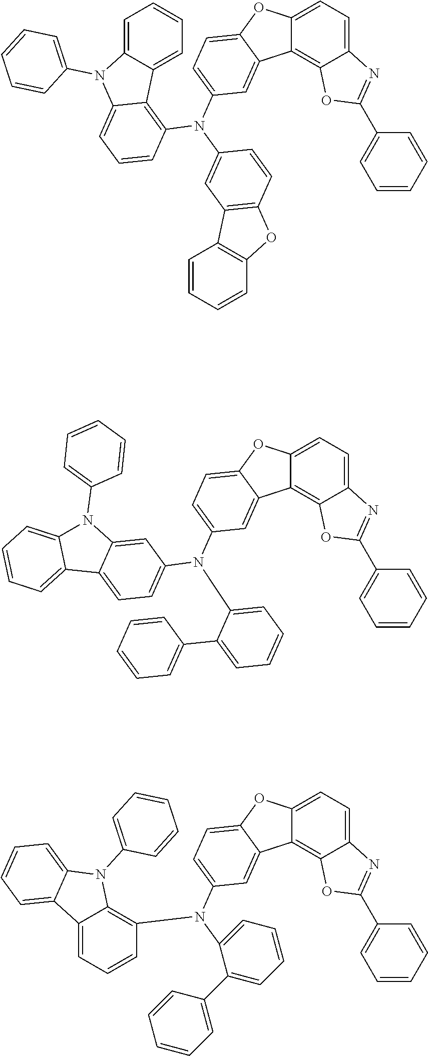 [SMILES: c1ccc(-c2nc3ccc4oc5ccc(N(c6ccc7oc8ccccc8c7c6)c6cccc7c6c6ccccc6n7-c6ccccc6)cc5c4c3o2)cc1 ; c1ccc(-c2nc3ccc4oc5ccc(N(c6ccc7c8ccccc8n(-c8ccccc8)c7c6)c6ccccc6-c6ccccc6)cc5c4c3o2)cc1 ; c1ccc(-c2nc3ccc4oc5ccc(N(c6ccccc6-c6ccccc6)c6cccc7c8ccccc8n(-c8ccccc8)c67)cc5c4c3o2)cc1]

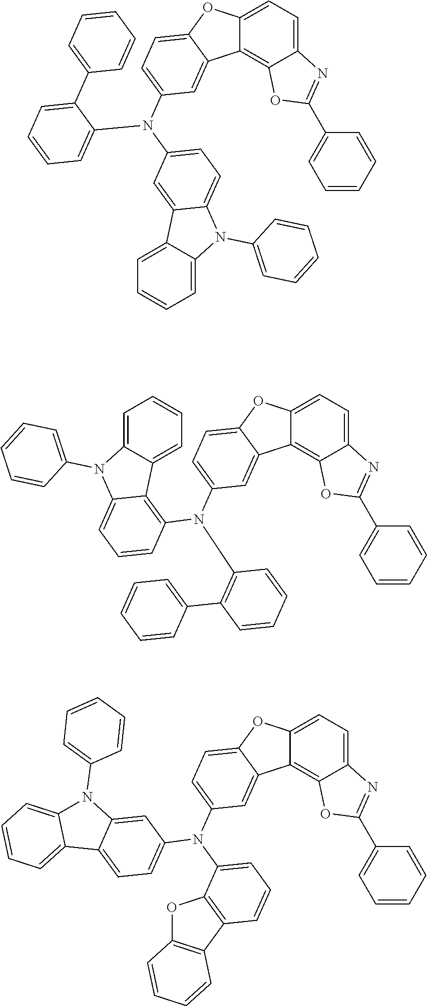 [SMILES: c1ccc(-c2nc3ccc4oc5ccc(N(c6ccc7c(c6)c6ccccc6n7-c6ccccc6)c6ccccc6-c6ccccc6)cc5c4c3o2)cc1 ; c1ccc(-c2nc3ccc4oc5ccc(N(c6ccccc6-c6ccccc6)c6cccc7c6c6ccccc6n7-c6ccccc6)cc5c4c3o2)cc1 ; c1ccc(-c2nc3ccc4oc5ccc(N(c6ccc7c8ccccc8n(-c8ccccc8)c7c6)c6cccc7c6oc6ccccc67)cc5c4c3o2)cc1]

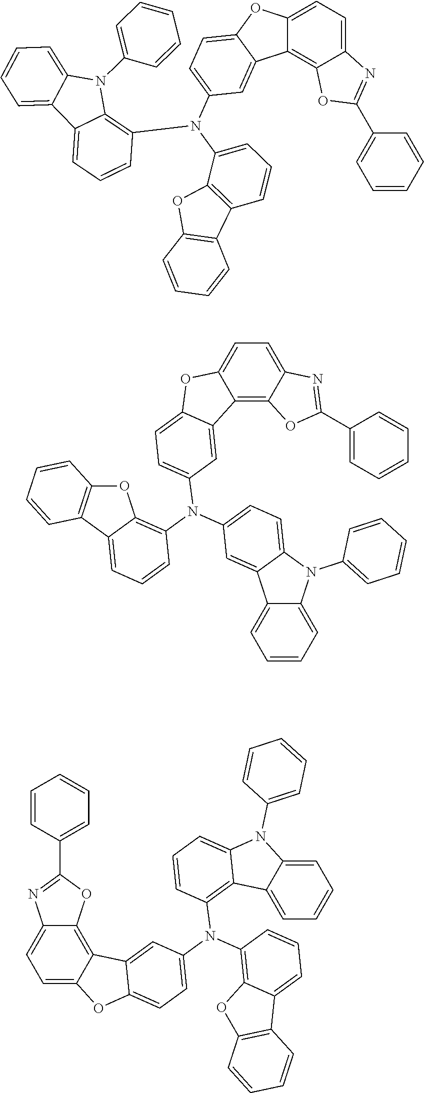 [SMILES: c1ccc(-c2nc3ccc4oc5ccc(N(c6cccc7c6oc6ccccc67)c6cccc7c8ccccc8n(-c8ccccc8)c67)cc5c4c3o2)cc1 ; c1ccc(-c2nc3ccc4oc5ccc(N(c6ccc7c(c6)c6ccccc6n7-c6ccccc6)c6cccc7c6oc6ccccc67)cc5c4c3o2)cc1 ; c1ccc(-c2nc3ccc4oc5ccc(N(c6cccc7c6oc6ccccc67)c6cccc7c6c6ccccc6n7-c6ccccc6)cc5c4c3o2)cc1]

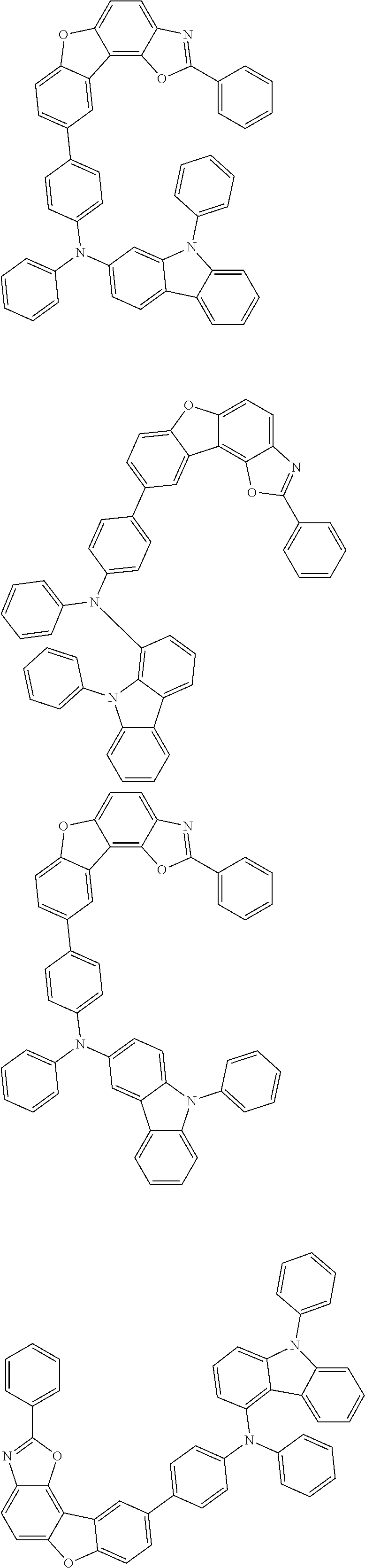

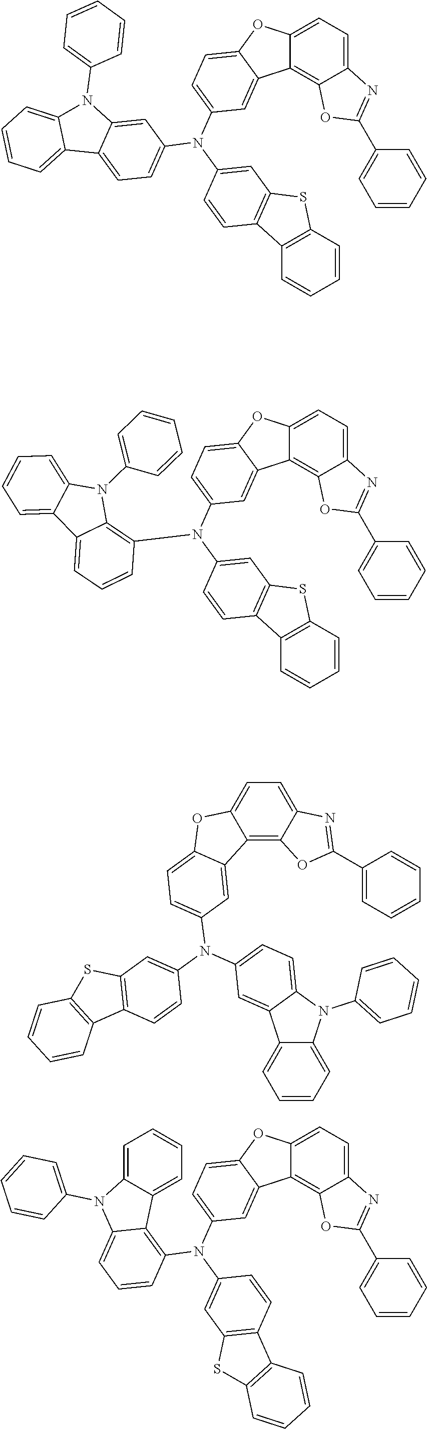

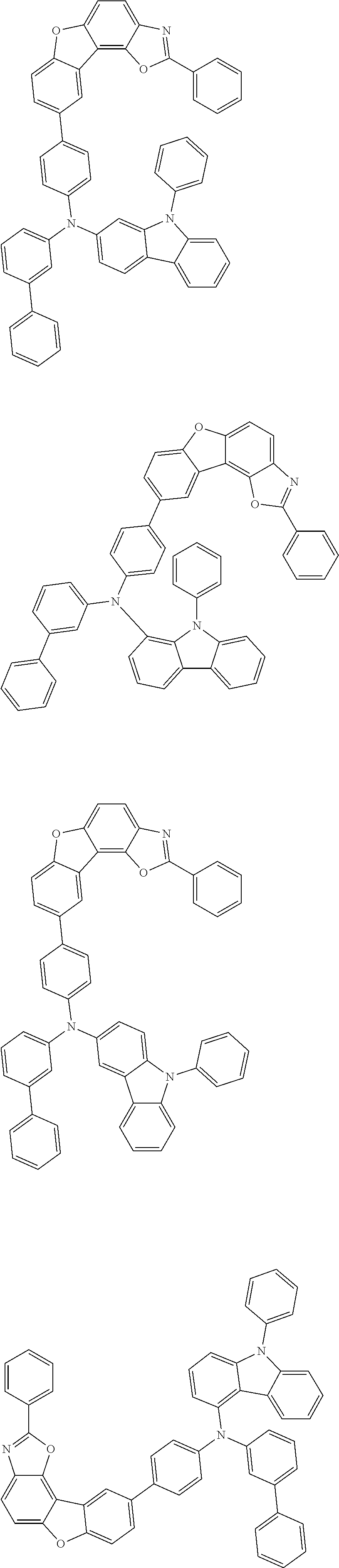 [SMILES: c1ccc(-c2cccc(N(c3ccc(-c4ccc5oc6ccc7nc(-c8ccccc8)oc7c6c5c4)cc3)c3ccc4c5ccccc5n(-c5ccccc5)c4c3)c2)cc1 ; c1ccc(-c2cccc(N(c3ccc(-c4ccc5oc6ccc7nc(-c8ccccc8)oc7c6c5c4)cc3)c3cccc4c5ccccc5n(-c5ccccc5)c34)c2)cc1 ; c1ccc(-c2cccc(N(c3ccc(-c4ccc5oc6ccc7nc(-c8ccccc8)oc7c6c5c4)cc3)c3ccc4c(c3)c3ccccc3n4-c3ccccc3)c2)cc1 ; c1ccc(-c2cccc(N(c3ccc(-c4ccc5oc6ccc7nc(-c8ccccc8)oc7c6c5c4)cc3)c3cccc4c3c3ccccc3n4-c3ccccc3)c2)cc1]

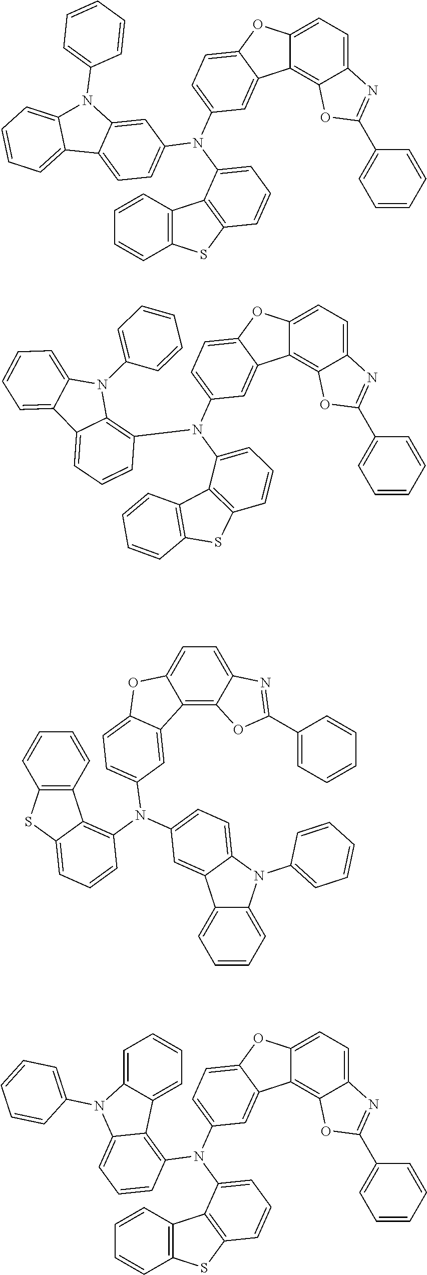

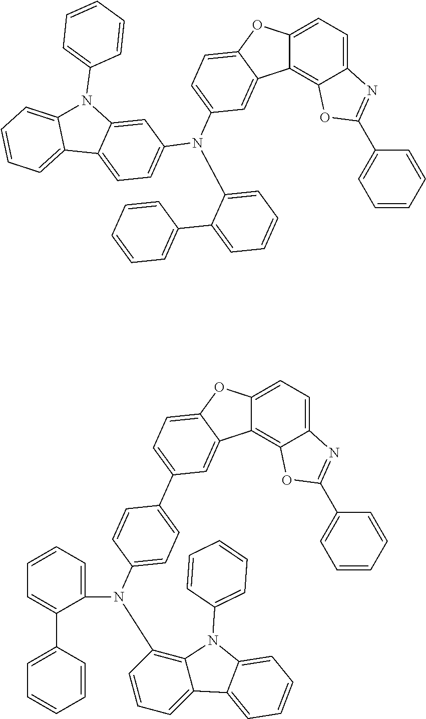 [SMILES: c1ccc(-c2nc3ccc4oc5ccc(N(c6ccc7c8ccccc8n(-c8ccccc8)c7c6)c6ccccc6-c6ccccc6)cc5c4c3o2)cc1 ; c1ccc(-c2nc3ccc4oc5ccc(-c6ccc(N(c7ccccc7-c7ccccc7)c7cccc8c9ccccc9n(-c9ccccc9)c78)cc6)cc5c4c3o2)cc1]

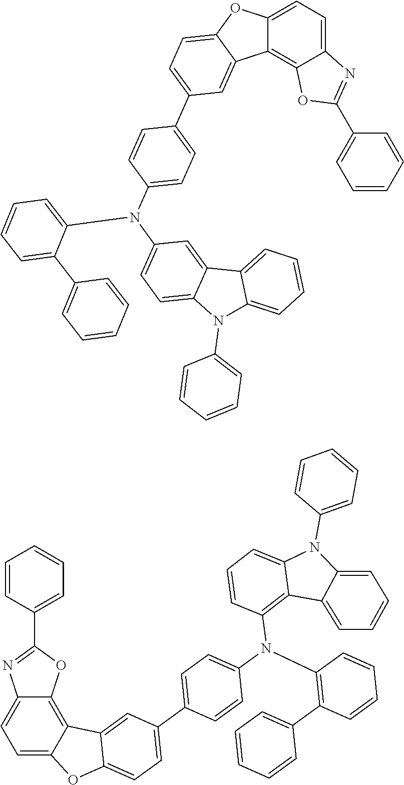 [SMILES: c1ccc(-c2nc3ccc4oc5ccc(-c6ccc(N(c7ccc8c(c7)c7ccccc7n8-c7ccccc7)c7ccccc7-c7ccccc7)cc6)cc5c4c3o2)cc1 ; c1ccc(-c2nc3ccc4oc5ccc(-c6ccc(N(c7ccccc7-c7ccccc7)c7cccc8c7c7ccccc7n8-c7ccccc7)cc6)cc5c4c3o2)cc1]

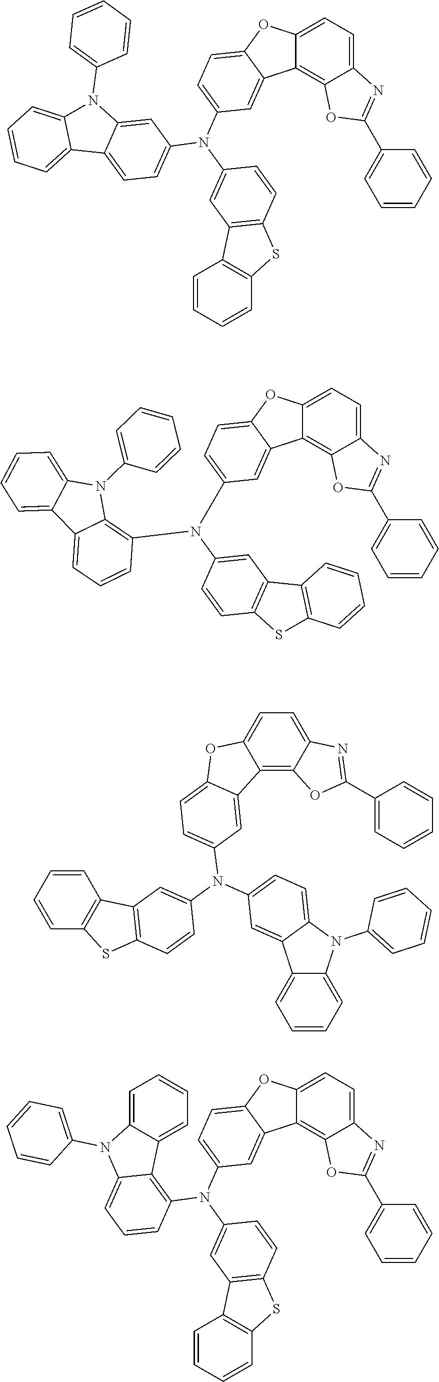

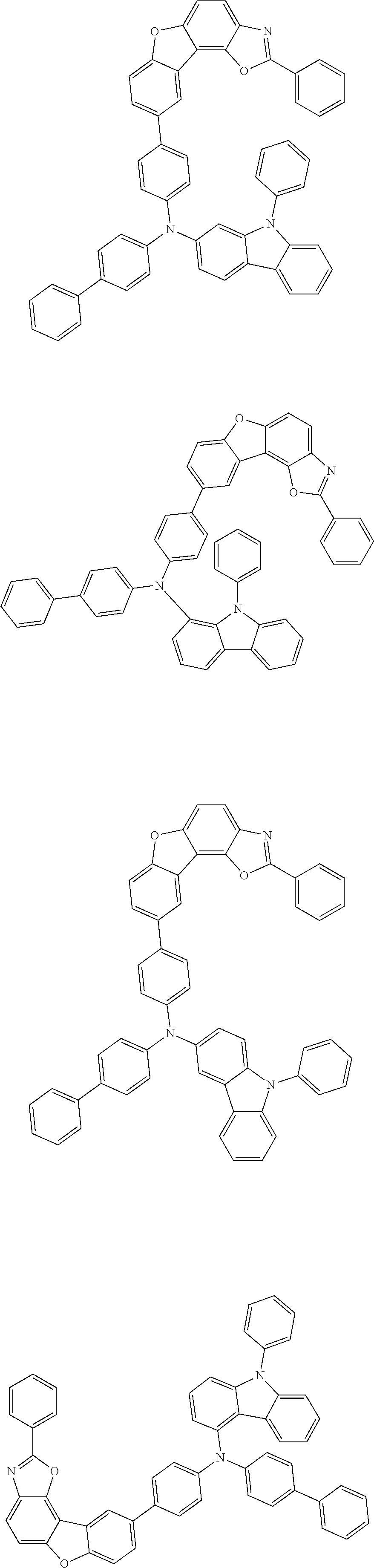 [SMILES: c1ccc(-c2ccc(N(c3ccc(-c4ccc5oc6ccc7nc(-c8ccccc8)oc7c6c5c4)cc3)c3ccc4c5ccccc5n(-c5ccccc5)c4c3)cc2)cc1 ; c1ccc(-c2ccc(N(c3ccc(-c4ccc5oc6ccc7nc(-c8ccccc8)oc7c6c5c4)cc3)c3cccc4c5ccccc5n(-c5ccccc5)c34)cc2)cc1 ; c1ccc(-c2ccc(N(c3ccc(-c4ccc5oc6ccc7nc(-c8ccccc8)oc7c6c5c4)cc3)c3ccc4c(c3)c3ccccc3n4-c3ccccc3)cc2)cc1 ; c1ccc(-c2ccc(N(c3ccc(-c4ccc5oc6ccc7nc(-c8ccccc8)oc7c6c5c4)cc3)c3cccc4c3c3ccccc3n4-c3ccccc3)cc2)cc1]

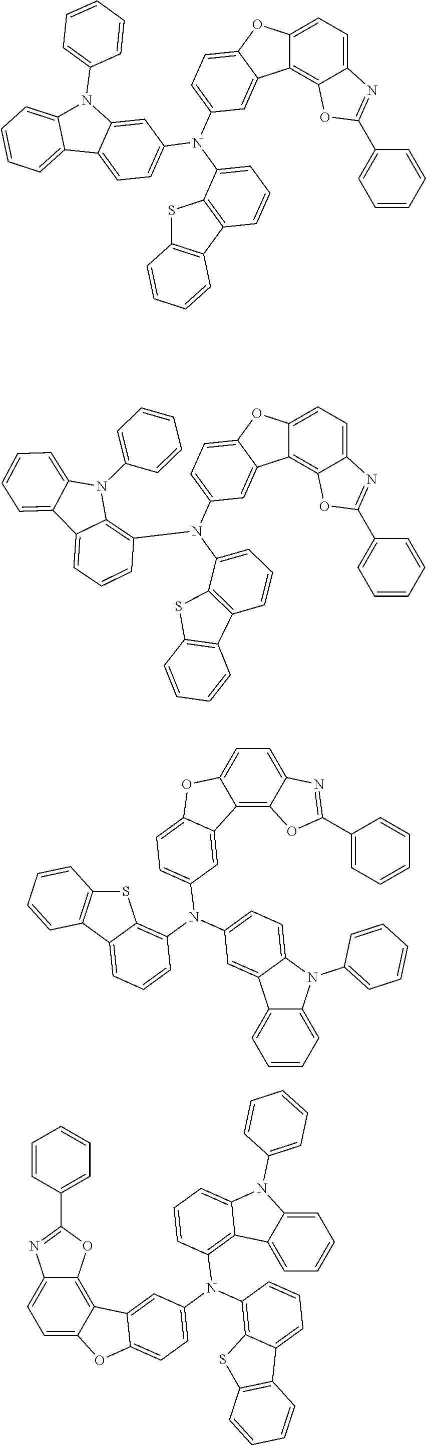

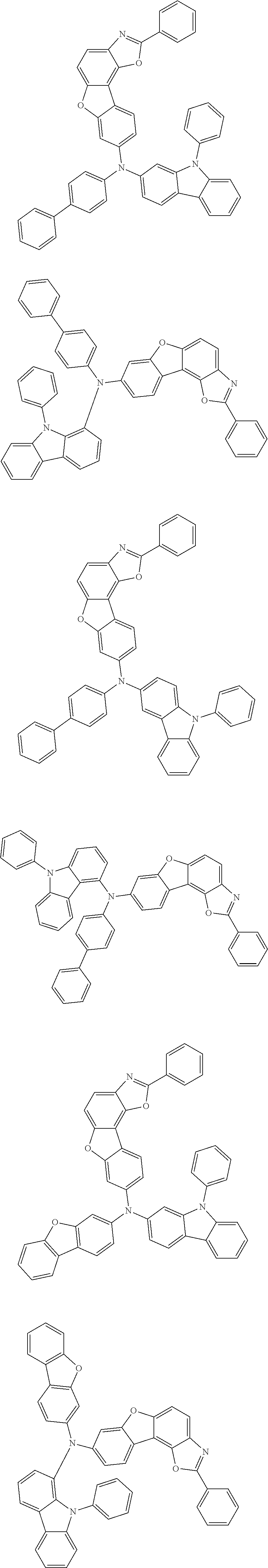

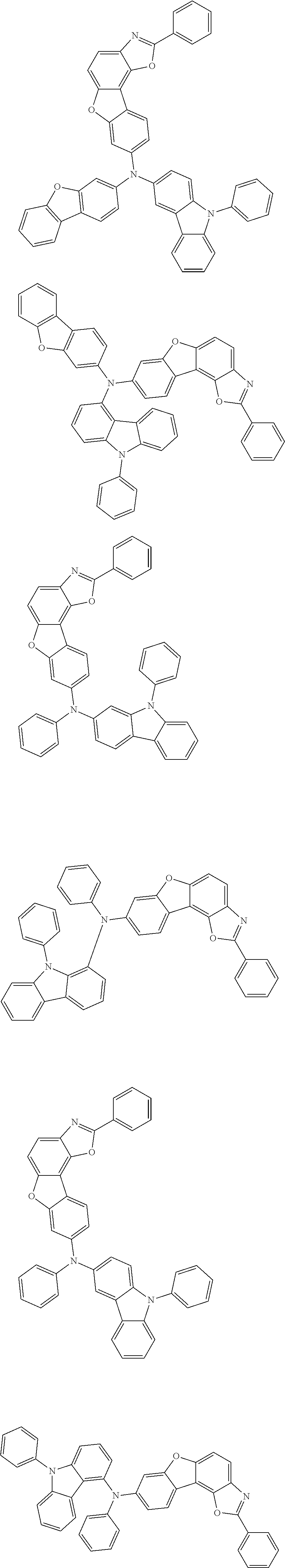

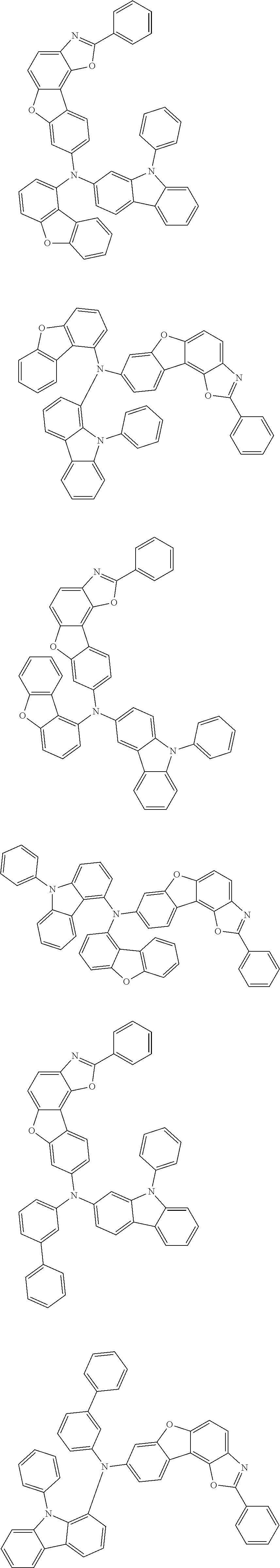

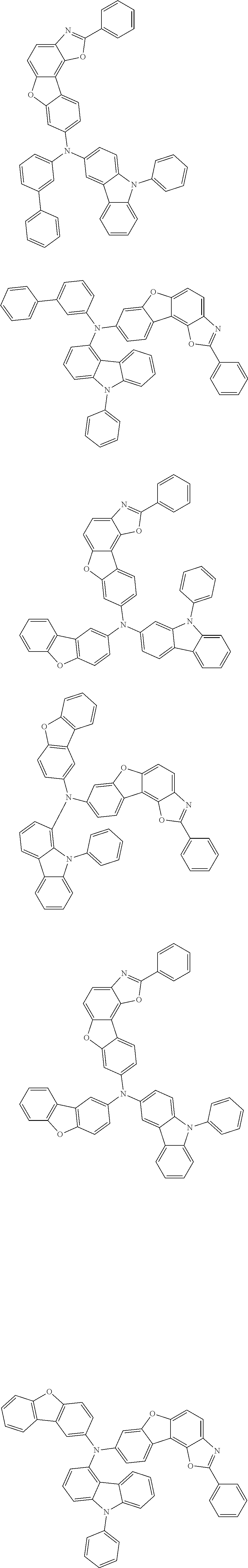

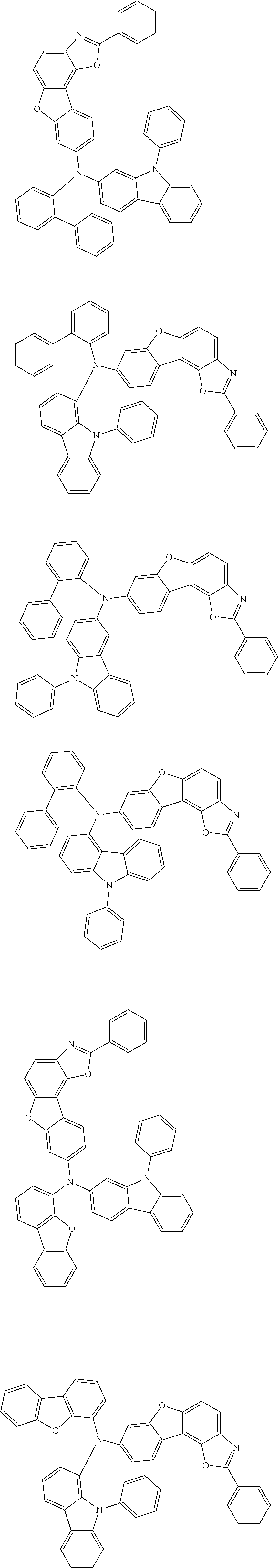

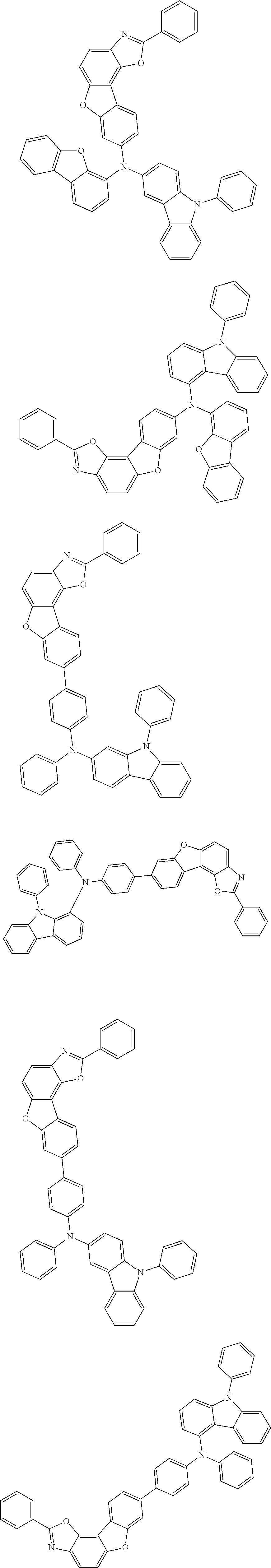

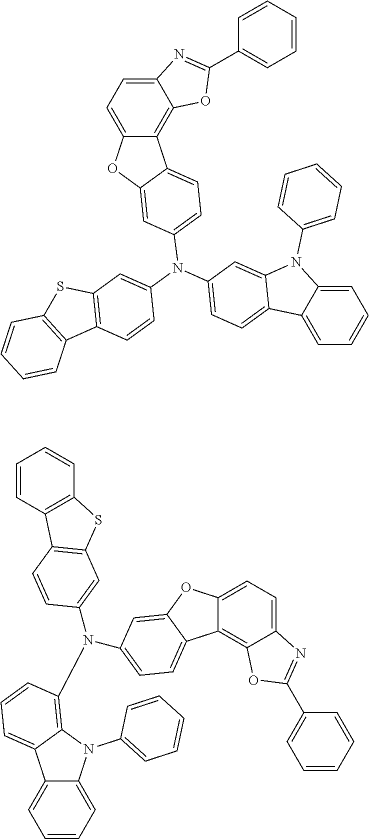 [SMILES: c1ccc(-c2nc3ccc4oc5cc(N(c6ccc7c(c6)sc6ccccc67)c6ccc7c8ccccc8n(-c8ccccc8)c7c6)ccc5c4c3o2)cc1 ; c1ccc(-c2nc3ccc4oc5cc(N(c6ccc7c(c6)sc6ccccc67)c6cccc7c8ccccc8n(-c8ccccc8)c67)ccc5c4c3o2)cc1]

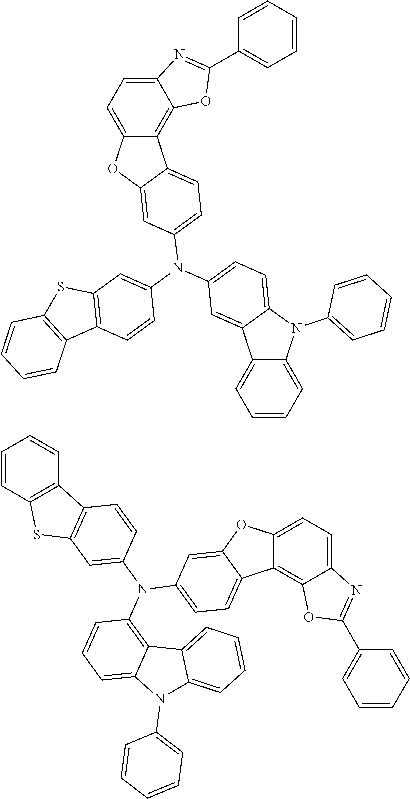 [SMILES: c1ccc(-c2nc3ccc4oc5cc(N(c6ccc7c(c6)sc6ccccc67)c6ccc7c(c6)c6ccccc6n7-c6ccccc6)ccc5c4c3o2)cc1 ; c1ccc(-c2nc3ccc4oc5cc(N(c6ccc7c(c6)sc6ccccc67)c6cccc7c6c6ccccc6n7-c6ccccc6)ccc5c4c3o2)cc1]

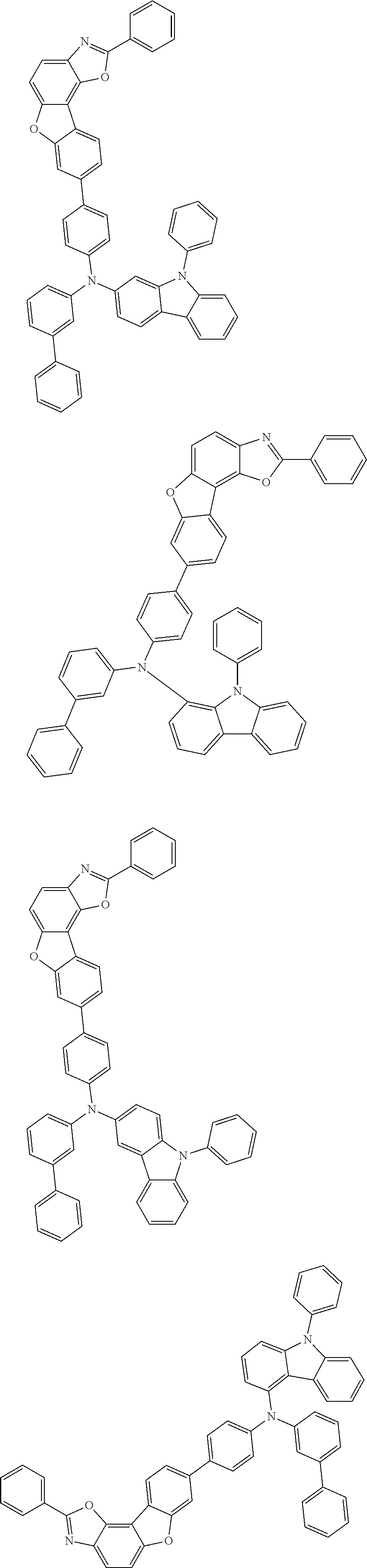 [SMILES: c1ccc(-c2cccc(N(c3ccc(-c4ccc5c(c4)oc4ccc6nc(-c7ccccc7)oc6c45)cc3)c3ccc4c5ccccc5n(-c5ccccc5)c4c3)c2)cc1 ; c1ccc(-c2cccc(N(c3ccc(-c4ccc5c(c4)oc4ccc6nc(-c7ccccc7)oc6c45)cc3)c3cccc4c5ccccc5n(-c5ccccc5)c34)c2)cc1 ; c1ccc(-c2cccc(N(c3ccc(-c4ccc5c(c4)oc4ccc6nc(-c7ccccc7)oc6c45)cc3)c3ccc4c(c3)c3ccccc3n4-c3ccccc3)c2)cc1 ; c1ccc(-c2cccc(N(c3ccc(-c4ccc5c(c4)oc4ccc6nc(-c7ccccc7)oc6c45)cc3)c3cccc4c3c3ccccc3n4-c3ccccc3)c2)cc1]

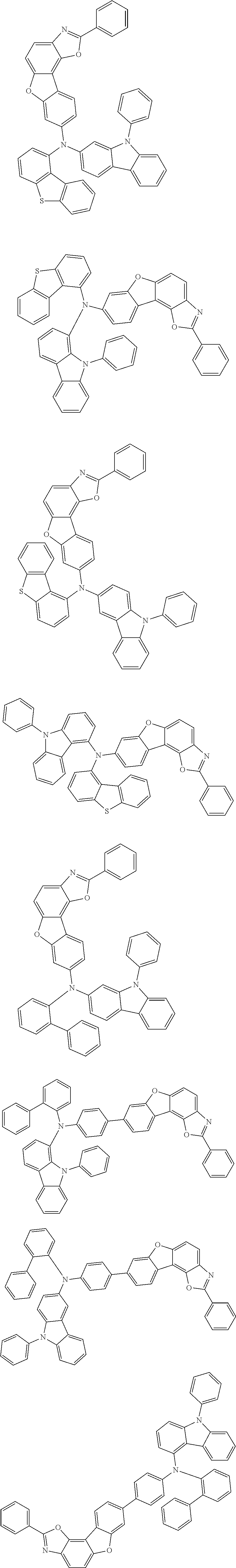

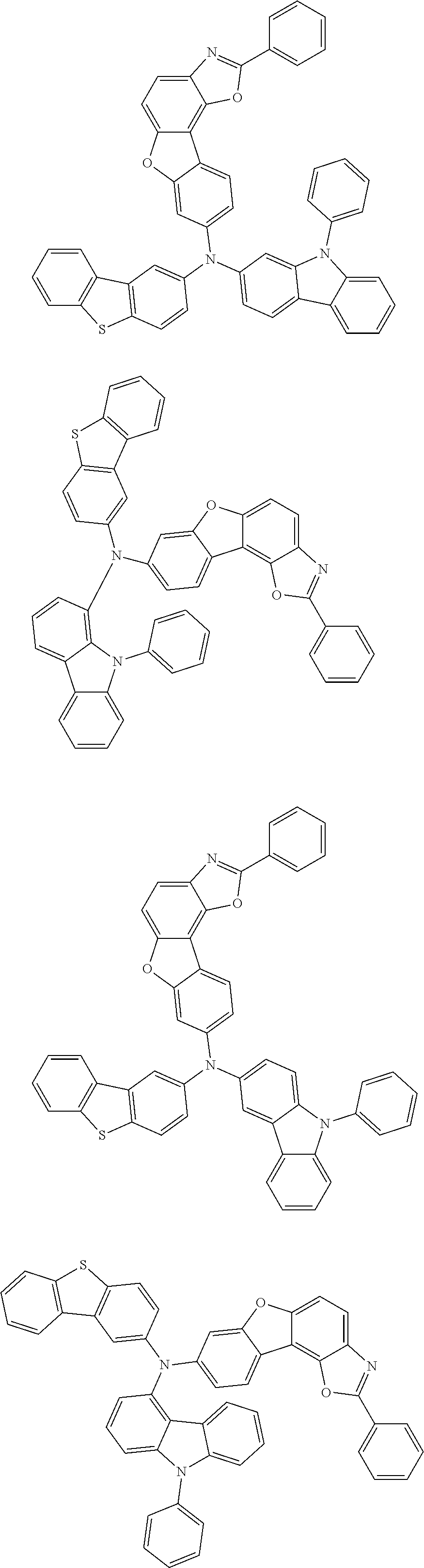 [SMILES: c1ccc(-c2nc3ccc4oc5cc(N(c6ccc7sc8ccccc8c7c6)c6ccc7c8ccccc8n(-c8ccccc8)c7c6)ccc5c4c3o2)cc1 ; c1ccc(-c2nc3ccc4oc5cc(N(c6ccc7sc8ccccc8c7c6)c6cccc7c8ccccc8n(-c8ccccc8)c67)ccc5c4c3o2)cc1 ; c1ccc(-c2nc3ccc4oc5cc(N(c6ccc7sc8ccccc8c7c6)c6ccc7c(c6)c6ccccc6n7-c6ccccc6)ccc5c4c3o2)cc1 ; c1ccc(-c2nc3ccc4oc5cc(N(c6ccc7sc8ccccc8c7c6)c6cccc7c6c6ccccc6n7-c6ccccc6)ccc5c4c3o2)cc1]

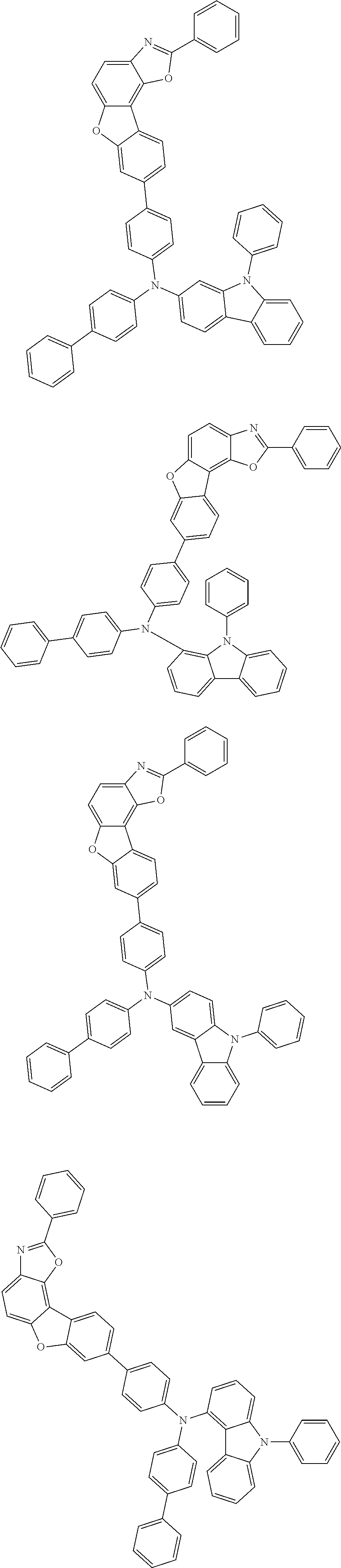

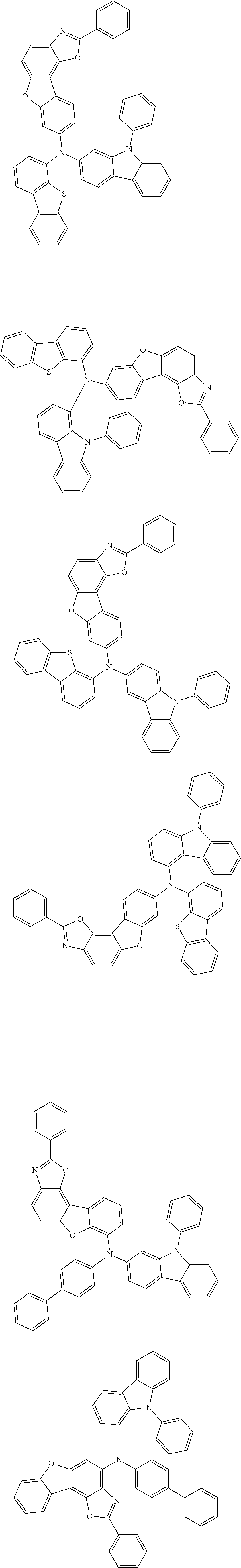

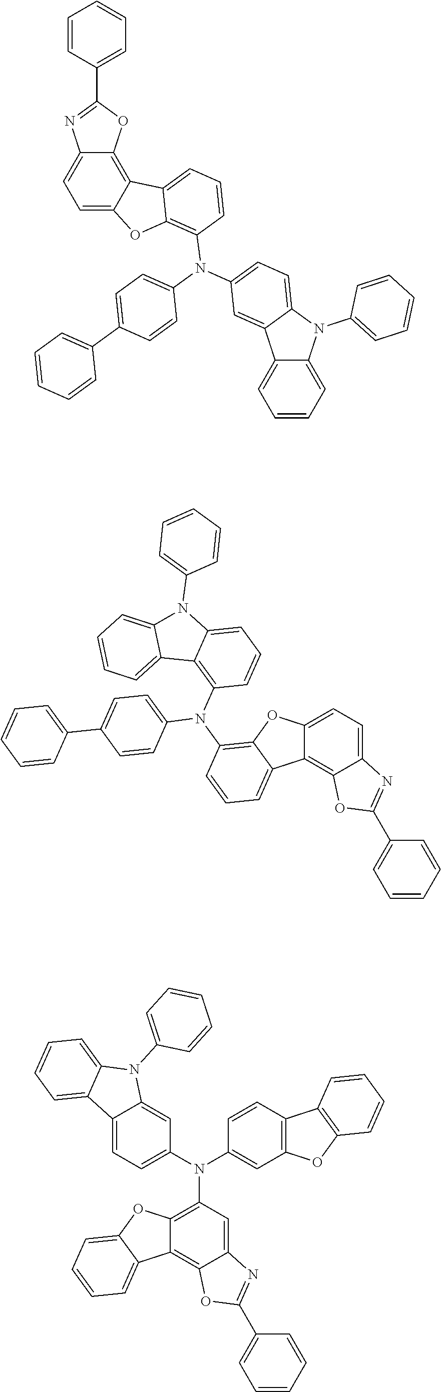 [SMILES: c1ccc(-c2ccc(N(c3ccc4c(c3)c3ccccc3n4-c3ccccc3)c3cccc4c3oc3ccc5nc(-c6ccccc6)oc5c34)cc2)cc1 ; c1ccc(-c2ccc(N(c3cccc4c3oc3ccc5nc(-c6ccccc6)oc5c34)c3cccc4c3c3ccccc3n4-c3ccccc3)cc2)cc1 ; c1ccc(-c2nc3cc(N(c4ccc5c(c4)oc4ccccc45)c4ccc5c6ccccc6n(-c6ccccc6)c5c4)c4oc5ccccc5c4c3o2)cc1]

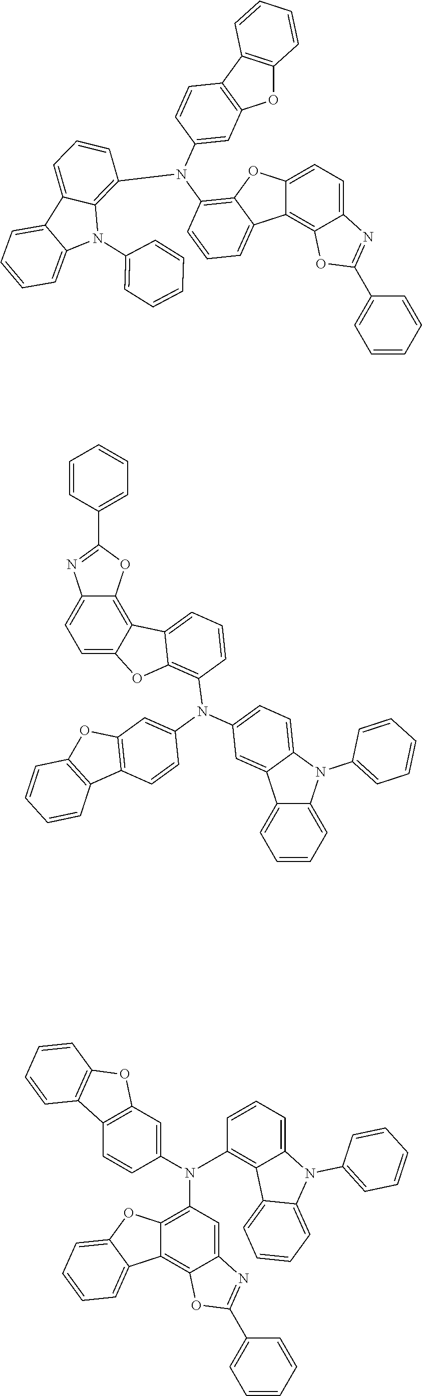 [SMILES: c1ccc(-c2nc3ccc4oc5c(N(c6ccc7c(c6)oc6ccccc67)c6cccc7c8ccccc8n(-c8ccccc8)c67)cccc5c4c3o2)cc1 ; c1ccc(-c2nc3ccc4oc5c(N(c6ccc7c(c6)oc6ccccc67)c6ccc7c(c6)c6ccccc6n7-c6ccccc6)cccc5c4c3o2)cc1 ; c1ccc(-c2nc3cc(N(c4ccc5c(c4)oc4ccccc45)c4cccc5c4c4ccccc4n5-c4ccccc4)c4oc5ccccc5c4c3o2)cc1]

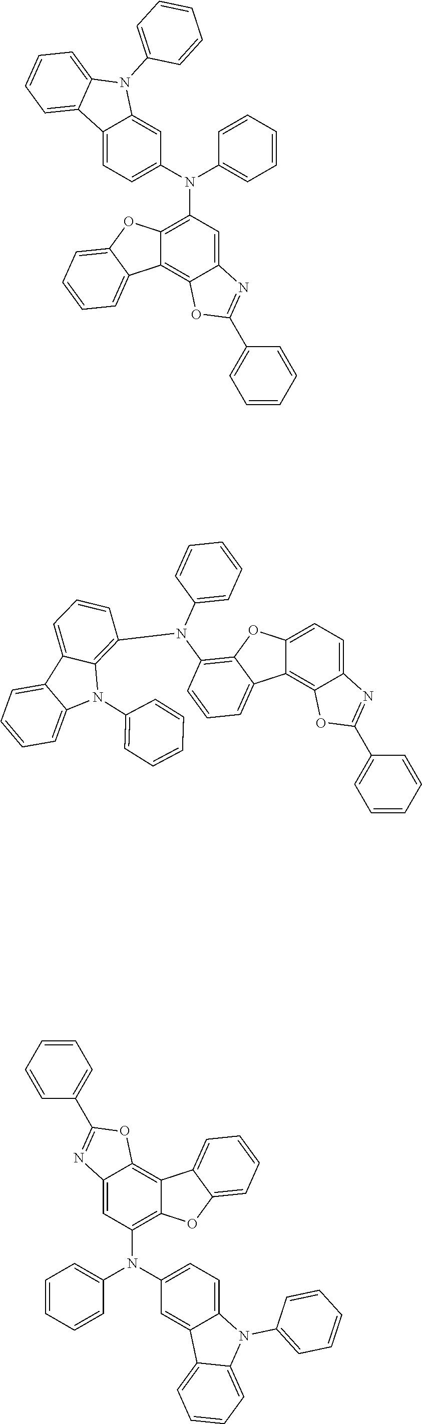 [SMILES: c1ccc(-c2nc3cc(N(c4ccccc4)c4ccc5c6ccccc6n(-c6ccccc6)c5c4)c4oc5ccccc5c4c3o2)cc1 ; c1ccc(-c2nc3ccc4oc5c(N(c6ccccc6)c6cccc7c8ccccc8n(-c8ccccc8)c67)cccc5c4c3o2)cc1 ; c1ccc(-c2nc3cc(N(c4ccccc4)c4ccc5c(c4)c4ccccc4n5-c4ccccc4)c4oc5ccccc5c4c3o2)cc1]

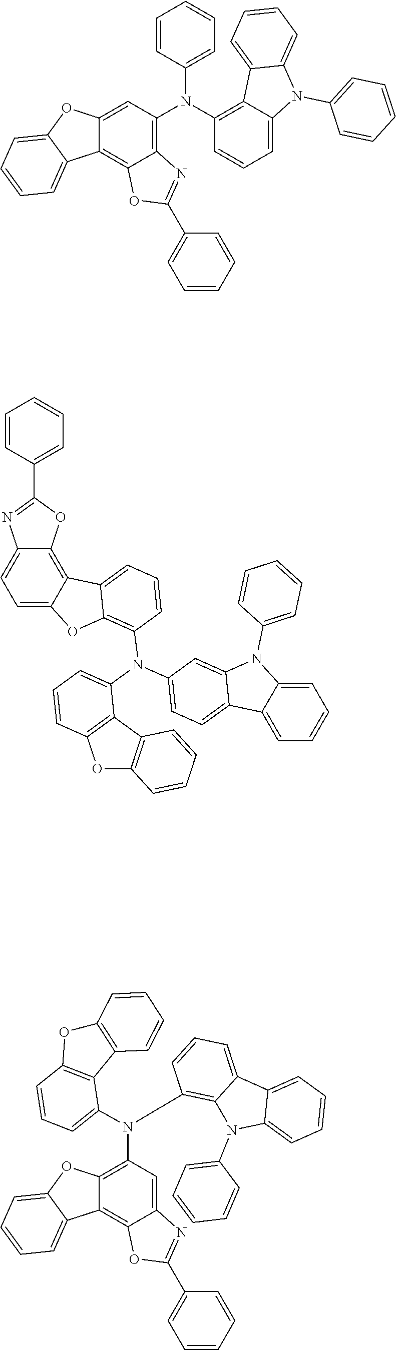 [SMILES: c1ccc(-c2nc3c(N(c4ccccc4)c4cccc5c4c4ccccc4n5-c4ccccc4)cc4oc5ccccc5c4c3o2)cc1 ; c1ccc(-c2nc3ccc4oc5c(N(c6ccc7c8ccccc8n(-c8ccccc8)c7c6)c6cccc7oc8ccccc8c67)cccc5c4c3o2)cc1 ; c1ccc(-c2nc3cc(N(c4cccc5oc6ccccc6c45)c4cccc5c6ccccc6n(-c6ccccc6)c45)c4oc5ccccc5c4c3o2)cc1]

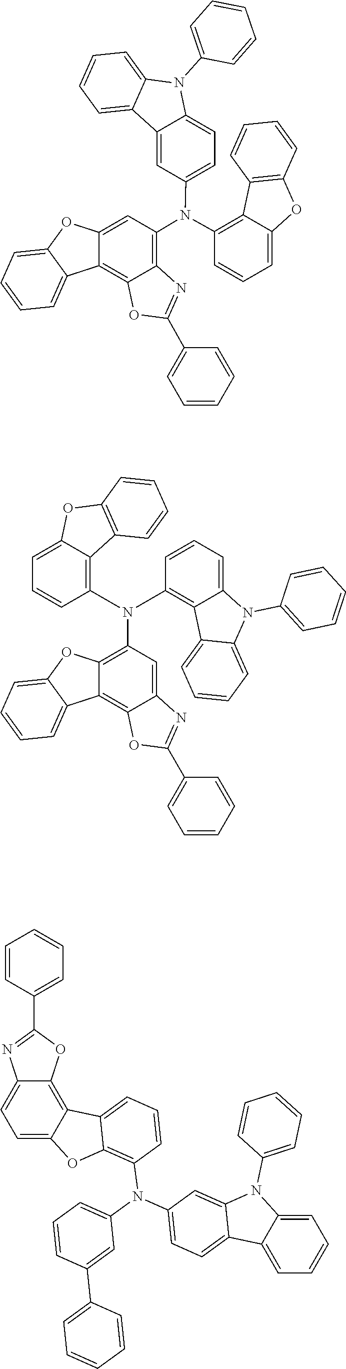 [SMILES: c1ccc(-c2nc3c(N(c4ccc5c(c4)c4ccccc4n5-c4ccccc4)c4cccc5oc6ccccc6c45)cc4oc5ccccc5c4c3o2)cc1 ; c1ccc(-c2nc3cc(N(c4cccc5oc6ccccc6c45)c4cccc5c4c4ccccc4n5-c4ccccc4)c4oc5ccccc5c4c3o2)cc1 ; c1ccc(-c2cccc(N(c3ccc4c5ccccc5n(-c5ccccc5)c4c3)c3cccc4c3oc3ccc5nc(-c6ccccc6)oc5c34)c2)cc1]

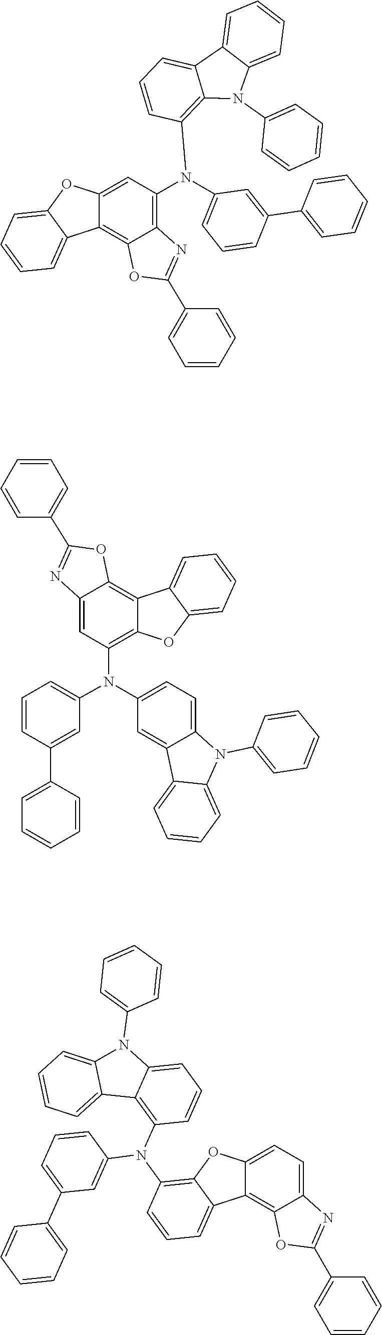 [SMILES: c1ccc(-c2cccc(N(c3cc4oc5ccccc5c4c4oc(-c5ccccc5)nc34)c3cccc4c5ccccc5n(-c5ccccc5)c34)c2)cc1 ; c1ccc(-c2cccc(N(c3ccc4c(c3)c3ccccc3n4-c3ccccc3)c3cc4nc(-c5ccccc5)oc4c4c3oc3ccccc34)c2)cc1 ; c1ccc(-c2cccc(N(c3cccc4c3oc3ccc5nc(-c6ccccc6)oc5c34)c3cccc4c3c3ccccc3n4-c3ccccc3)c2)cc1]

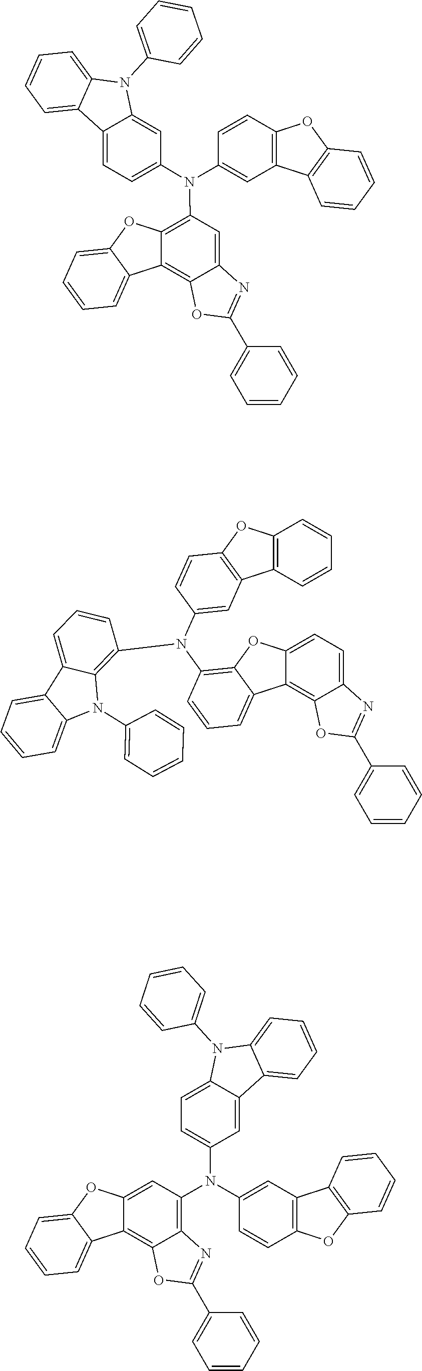 [SMILES: c1ccc(-c2nc3cc(N(c4ccc5oc6ccccc6c5c4)c4ccc5c6ccccc6n(-c6ccccc6)c5c4)c4oc5ccccc5c4c3o2)cc1 ; c1ccc(-c2nc3ccc4oc5c(N(c6ccc7oc8ccccc8c7c6)c6cccc7c8ccccc8n(-c8ccccc8)c67)cccc5c4c3o2)cc1 ; c1ccc(-c2nc3c(N(c4ccc5oc6ccccc6c5c4)c4ccc5c(c4)c4ccccc4n5-c4ccccc4)cc4oc5ccccc5c4c3o2)cc1]

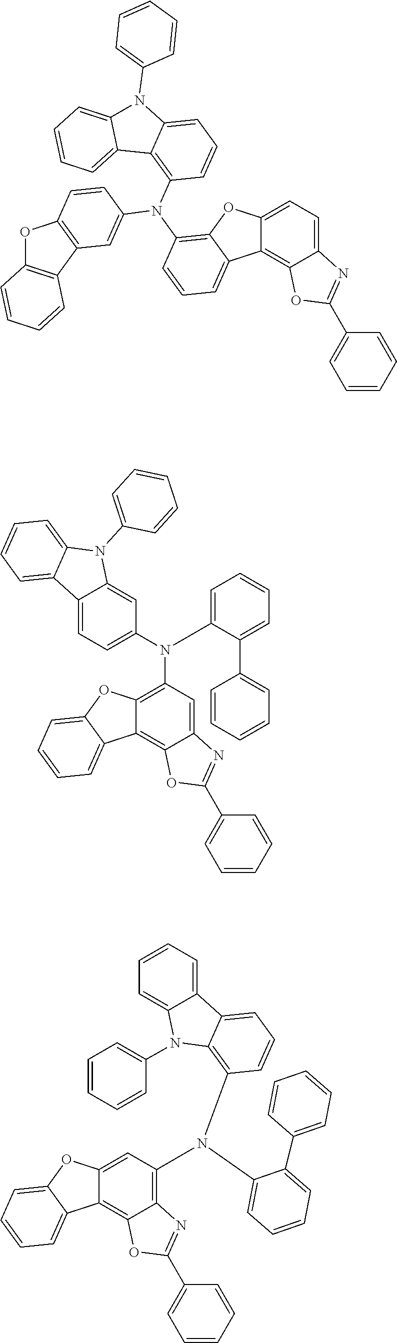 [SMILES: c1ccc(-c2nc3ccc4oc5c(N(c6ccc7oc8ccccc8c7c6)c6cccc7c6c6ccccc6n7-c6ccccc6)cccc5c4c3o2)cc1 ; c1ccc(-c2nc3cc(N(c4ccc5c6ccccc6n(-c6ccccc6)c5c4)c4ccccc4-c4ccccc4)c4oc5ccccc5c4c3o2)cc1 ; c1ccc(-c2nc3c(N(c4ccccc4-c4ccccc4)c4cccc5c6ccccc6n(-c6ccccc6)c45)cc4oc5ccccc5c4c3o2)cc1]

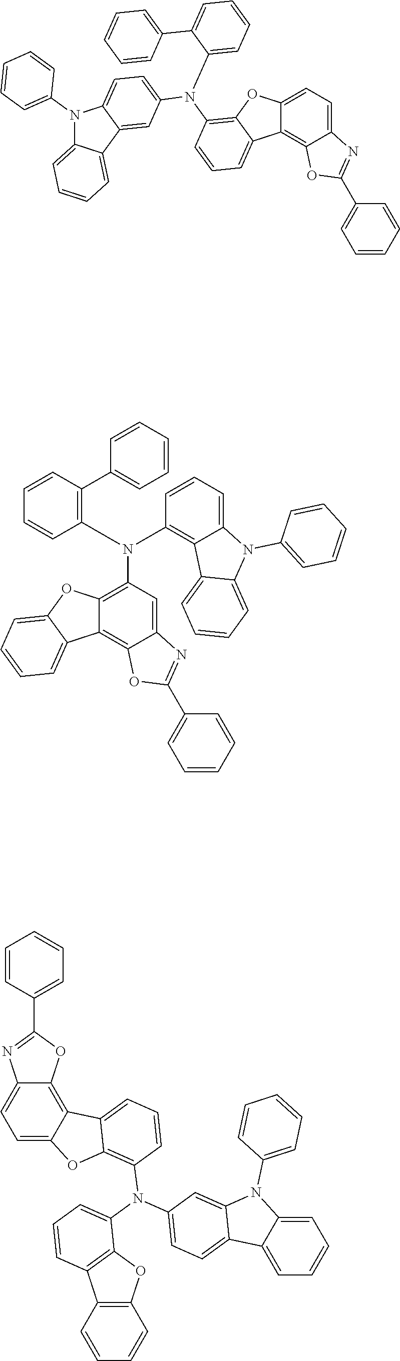 [SMILES: c1ccc(-c2nc3ccc4oc5c(N(c6ccc7c(c6)c6ccccc6n7-c6ccccc6)c6ccccc6-c6ccccc6)cccc5c4c3o2)cc1 ; c1ccc(-c2nc3cc(N(c4ccccc4-c4ccccc4)c4cccc5c4c4ccccc4n5-c4ccccc4)c4oc5ccccc5c4c3o2)cc1 ; c1ccc(-c2nc3ccc4oc5c(N(c6ccc7c8ccccc8n(-c8ccccc8)c7c6)c6cccc7c6oc6ccccc67)cccc5c4c3o2)cc1]

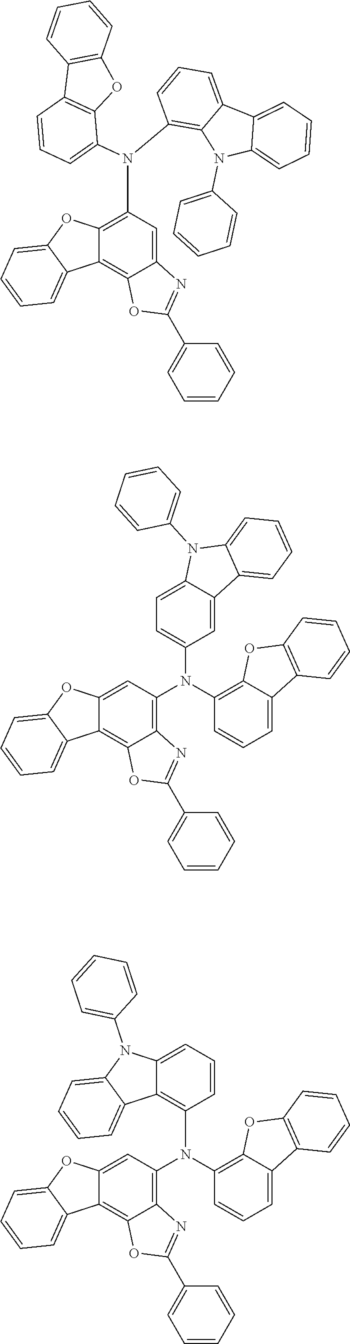 [SMILES: c1ccc(-c2nc3cc(N(c4cccc5c4oc4ccccc45)c4cccc5c6ccccc6n(-c6ccccc6)c45)c4oc5ccccc5c4c3o2)cc1 ; c1ccc(-c2nc3c(N(c4ccc5c(c4)c4ccccc4n5-c4ccccc4)c4cccc5c4oc4ccccc45)cc4oc5ccccc5c4c3o2)cc1 ; c1ccc(-c2nc3c(N(c4cccc5c4oc4ccccc45)c4cccc5c4c4ccccc4n5-c4ccccc4)cc4oc5ccccc5c4c3o2)cc1]

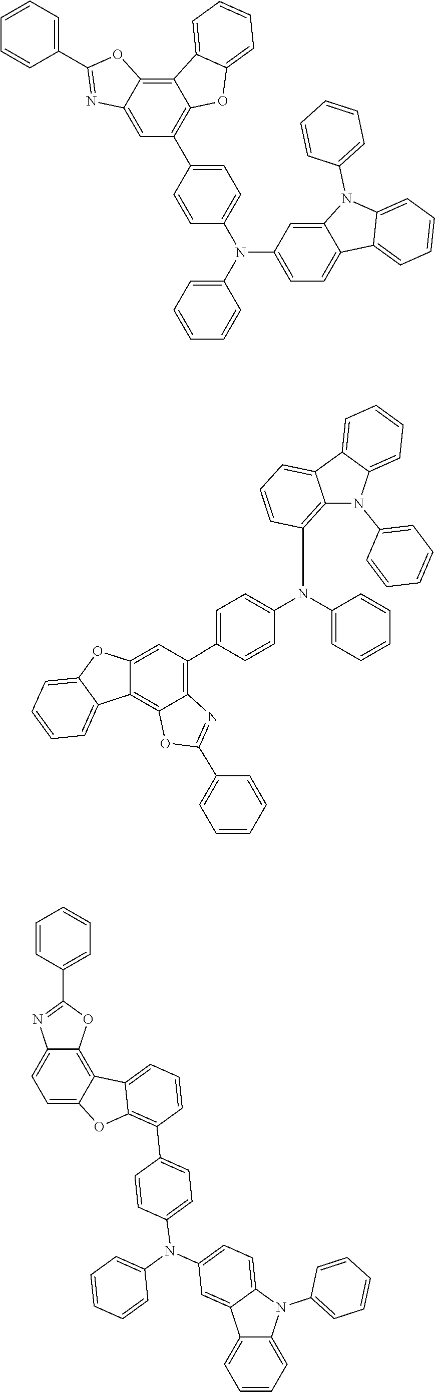 [SMILES: c1ccc(-c2nc3cc(-c4ccc(N(c5ccccc5)c5ccc6c7ccccc7n(-c7ccccc7)c6c5)cc4)c4oc5ccccc5c4c3o2)cc1 ; c1ccc(-c2nc3c(-c4ccc(N(c5ccccc5)c5cccc6c7ccccc7n(-c7ccccc7)c56)cc4)cc4oc5ccccc5c4c3o2)cc1 ; c1ccc(-c2nc3ccc4oc5c(-c6ccc(N(c7ccccc7)c7ccc8c(c7)c7ccccc7n8-c7ccccc7)cc6)cccc5c4c3o2)cc1]

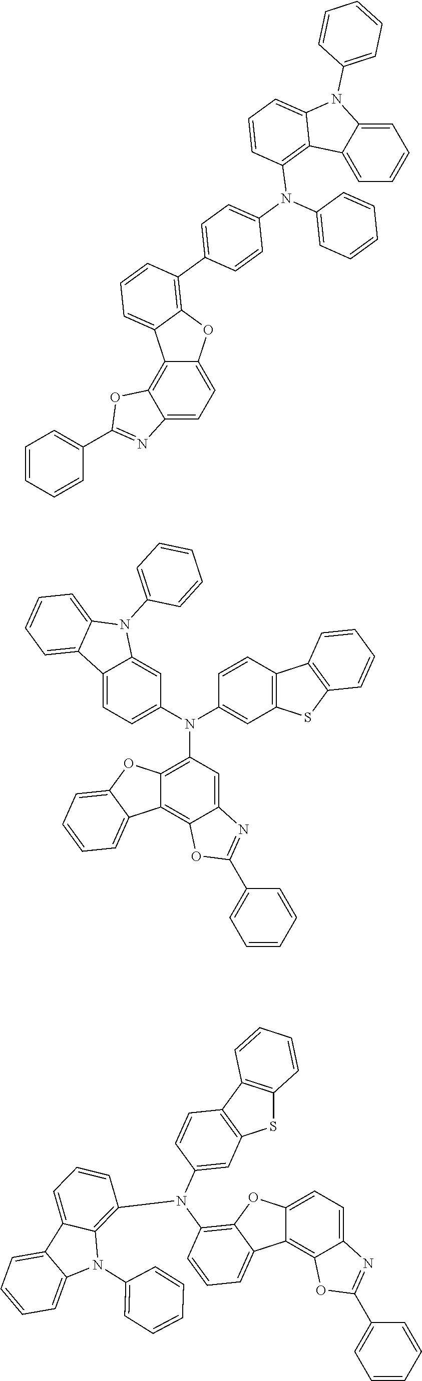 [SMILES: c1ccc(-c2nc3ccc4oc5c(-c6ccc(N(c7ccccc7)c7cccc8c7c7ccccc7n8-c7ccccc7)cc6)cccc5c4c3o2)cc1 ; c1ccc(-c2nc3cc(N(c4ccc5c(c4)sc4ccccc45)c4ccc5c6ccccc6n(-c6ccccc6)c5c4)c4oc5ccccc5c4c3o2)cc1 ; c1ccc(-c2nc3ccc4oc5c(N(c6ccc7c(c6)sc6ccccc67)c6cccc7c8ccccc8n(-c8ccccc8)c67)cccc5c4c3o2)cc1]

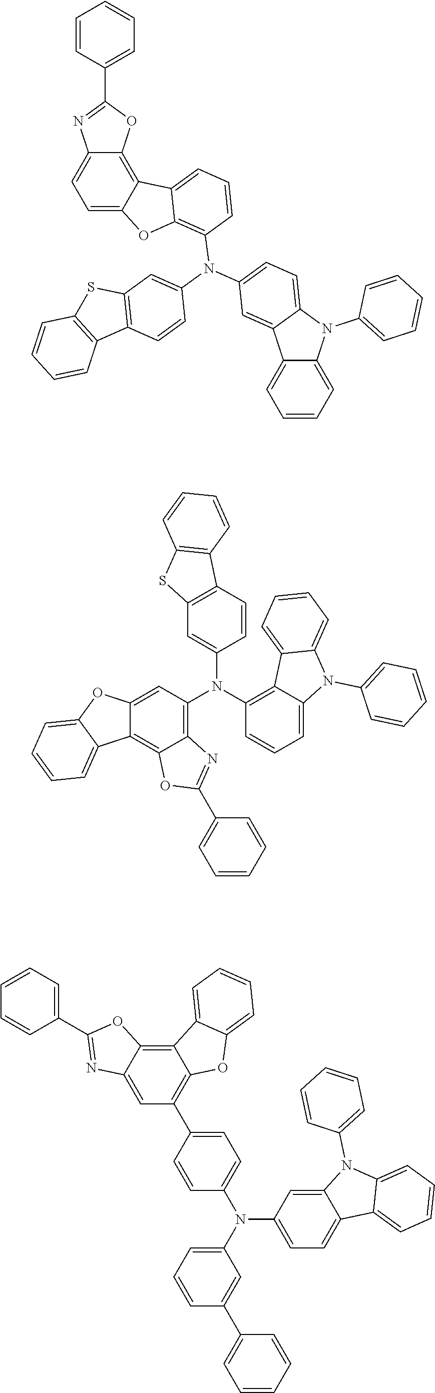 [SMILES: c1ccc(-c2nc3ccc4oc5c(N(c6ccc7c(c6)sc6ccccc67)c6ccc7c(c6)c6ccccc6n7-c6ccccc6)cccc5c4c3o2)cc1 ; c1ccc(-c2nc3c(N(c4ccc5c(c4)sc4ccccc45)c4cccc5c4c4ccccc4n5-c4ccccc4)cc4oc5ccccc5c4c3o2)cc1 ; c1ccc(-c2cccc(N(c3ccc(-c4cc5nc(-c6ccccc6)oc5c5c4oc4ccccc45)cc3)c3ccc4c5ccccc5n(-c5ccccc5)c4c3)c2)cc1]

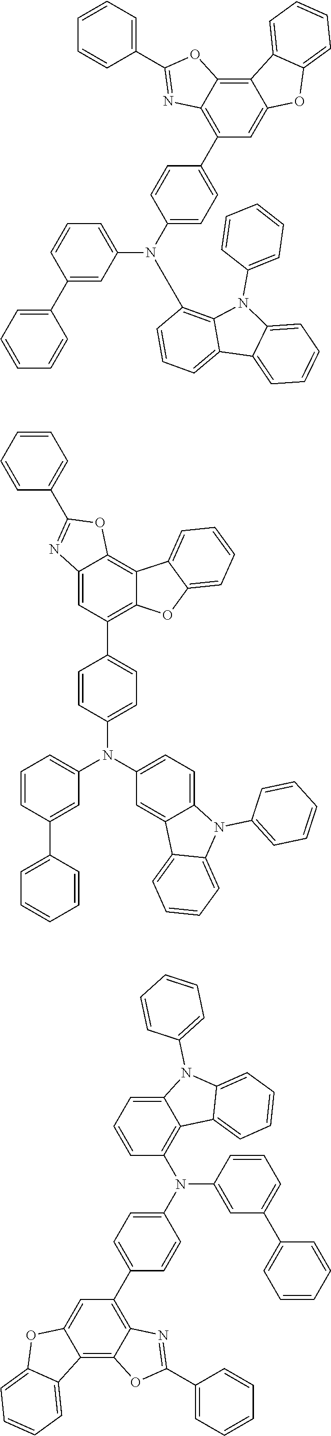 [SMILES: c1ccc(-c2cccc(N(c3ccc(-c4cc5oc6ccccc6c5c5oc(-c6ccccc6)nc45)cc3)c3cccc4c5ccccc5n(-c5ccccc5)c34)c2)cc1 ; c1ccc(-c2cccc(N(c3ccc(-c4cc5nc(-c6ccccc6)oc5c5c4oc4ccccc45)cc3)c3ccc4c(c3)c3ccccc3n4-c3ccccc3)c2)cc1 ; c1ccc(-c2cccc(N(c3ccc(-c4cc5oc6ccccc6c5c5oc(-c6ccccc6)nc45)cc3)c3cccc4c3c3ccccc3n4-c3ccccc3)c2)cc1]

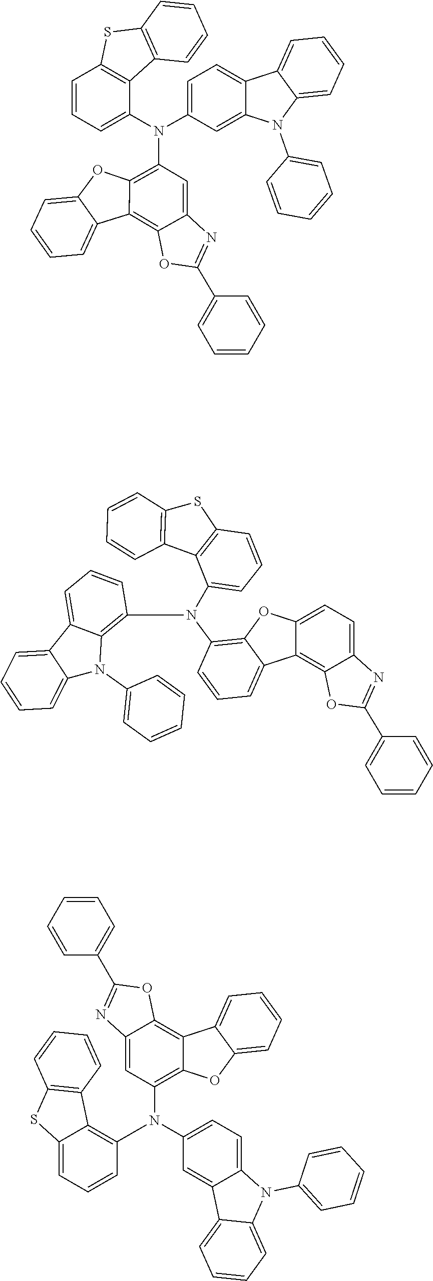 [SMILES: c1ccc(-c2nc3cc(N(c4ccc5c6ccccc6n(-c6ccccc6)c5c4)c4cccc5sc6ccccc6c45)c4oc5ccccc5c4c3o2)cc1 ; c1ccc(-c2nc3ccc4oc5c(N(c6cccc7sc8ccccc8c67)c6cccc7c8ccccc8n(-c8ccccc8)c67)cccc5c4c3o2)cc1 ; c1ccc(-c2nc3cc(N(c4ccc5c(c4)c4ccccc4n5-c4ccccc4)c4cccc5sc6ccccc6c45)c4oc5ccccc5c4c3o2)cc1]

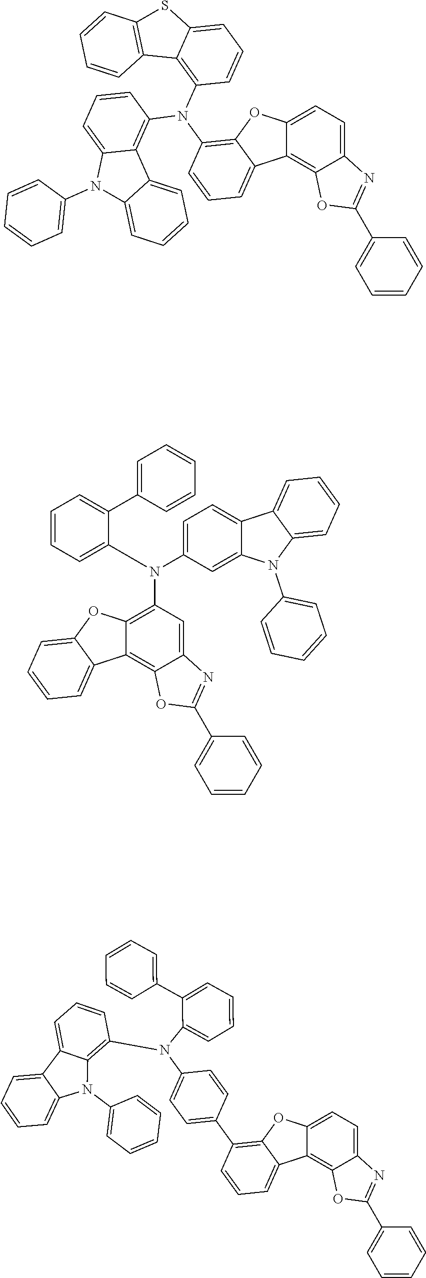 [SMILES: c1ccc(-c2nc3ccc4oc5c(N(c6cccc7sc8ccccc8c67)c6cccc7c6c6ccccc6n7-c6ccccc6)cccc5c4c3o2)cc1 ; c1ccc(-c2nc3cc(N(c4ccc5c6ccccc6n(-c6ccccc6)c5c4)c4ccccc4-c4ccccc4)c4oc5ccccc5c4c3o2)cc1 ; c1ccc(-c2nc3ccc4oc5c(-c6ccc(N(c7ccccc7-c7ccccc7)c7cccc8c9ccccc9n(-c9ccccc9)c78)cc6)cccc5c4c3o2)cc1]

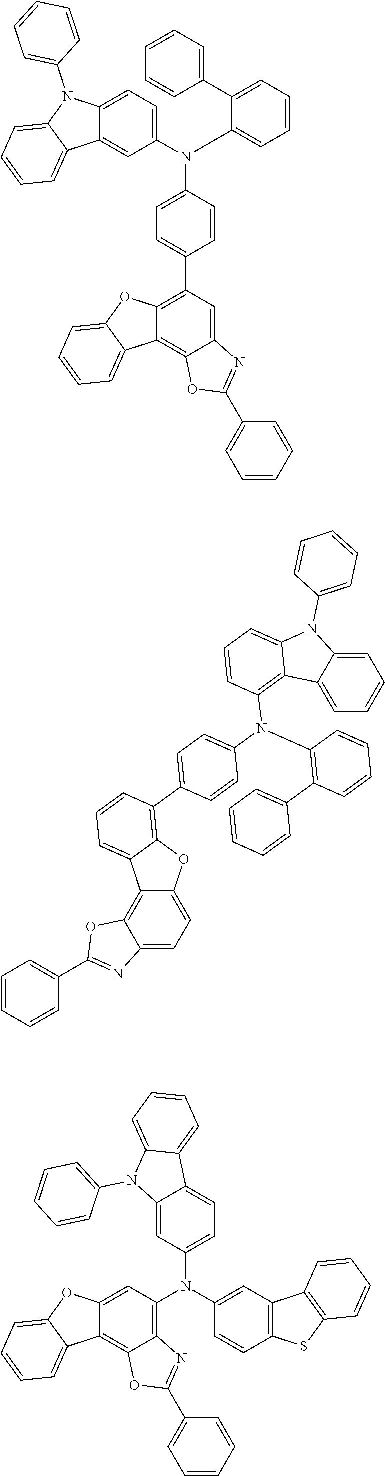 [SMILES: c1ccc(-c2nc3cc(-c4ccc(N(c5ccc6c(c5)c5ccccc5n6-c5ccccc5)c5ccccc5-c5ccccc5)cc4)c4oc5ccccc5c4c3o2)cc1 ; c1ccc(-c2nc3ccc4oc5c(-c6ccc(N(c7ccccc7-c7ccccc7)c7cccc8c7c7ccccc7n8-c7ccccc7)cc6)cccc5c4c3o2)cc1 ; c1ccc(-c2nc3c(N(c4ccc5sc6ccccc6c5c4)c4ccc5c6ccccc6n(-c6ccccc6)c5c4)cc4oc5ccccc5c4c3o2)cc1]

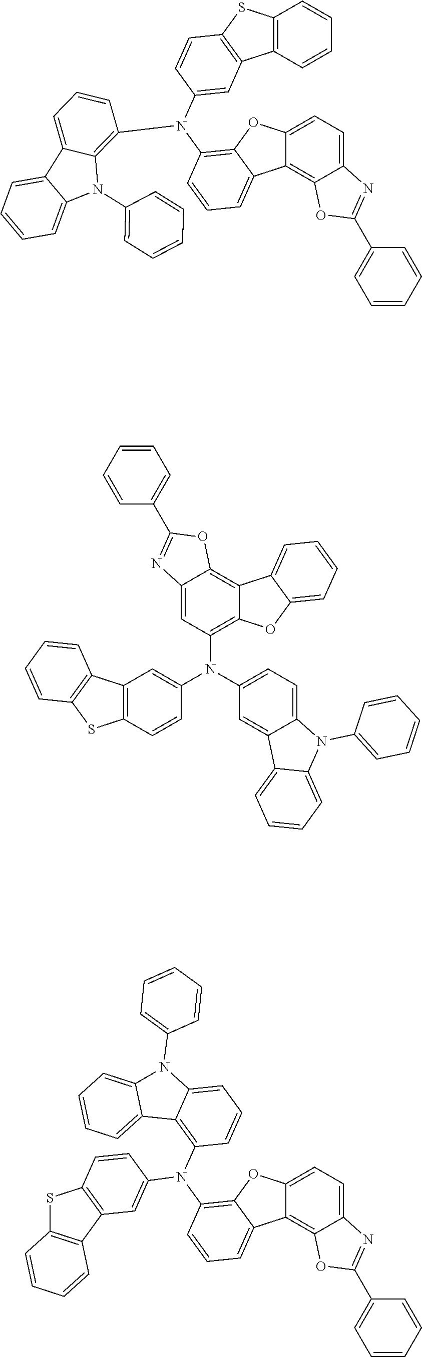 [SMILES: c1ccc(-c2nc3ccc4oc5c(N(c6ccc7sc8ccccc8c7c6)c6cccc7c8ccccc8n(-c8ccccc8)c67)cccc5c4c3o2)cc1 ; c1ccc(-c2nc3cc(N(c4ccc5sc6ccccc6c5c4)c4ccc5c(c4)c4ccccc4n5-c4ccccc4)c4oc5ccccc5c4c3o2)cc1 ; c1ccc(-c2nc3ccc4oc5c(N(c6ccc7sc8ccccc8c7c6)c6cccc7c6c6ccccc6n7-c6ccccc6)cccc5c4c3o2)cc1]

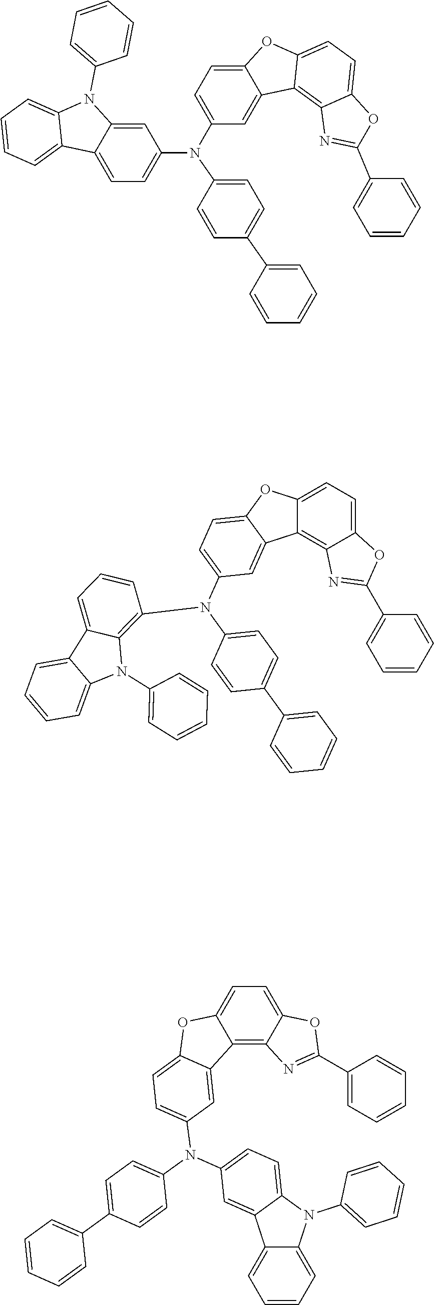 [SMILES: c1ccc(-c2ccc(N(c3ccc4oc5ccc6oc(-c7ccccc7)nc6c5c4c3)c3ccc4c5ccccc5n(-c5ccccc5)c4c3)cc2)cc1 ; c1ccc(-c2ccc(N(c3ccc4oc5ccc6oc(-c7ccccc7)nc6c5c4c3)c3cccc4c5ccccc5n(-c5ccccc5)c34)cc2)cc1 ; c1ccc(-c2ccc(N(c3ccc4oc5ccc6oc(-c7ccccc7)nc6c5c4c3)c3ccc4c(c3)c3ccccc3n4-c3ccccc3)cc2)cc1]

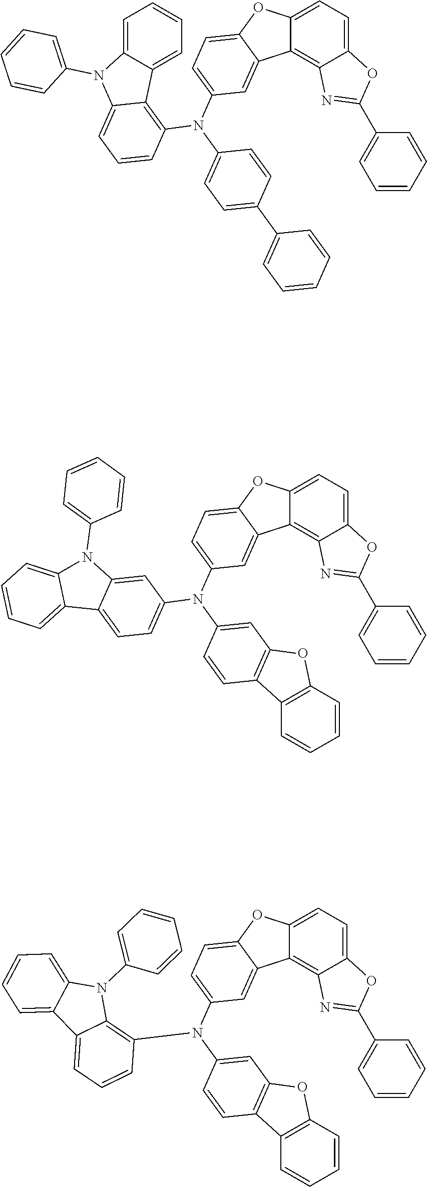 [SMILES: c1ccc(-c2ccc(N(c3ccc4oc5ccc6oc(-c7ccccc7)nc6c5c4c3)c3cccc4c3c3ccccc3n4-c3ccccc3)cc2)cc1 ; c1ccc(-c2nc3c(ccc4oc5ccc(N(c6ccc7c(c6)oc6ccccc67)c6ccc7c8ccccc8n(-c8ccccc8)c7c6)cc5c43)o2)cc1 ; c1ccc(-c2nc3c(ccc4oc5ccc(N(c6ccc7c(c6)oc6ccccc67)c6cccc7c8ccccc8n(-c8ccccc8)c67)cc5c43)o2)cc1]

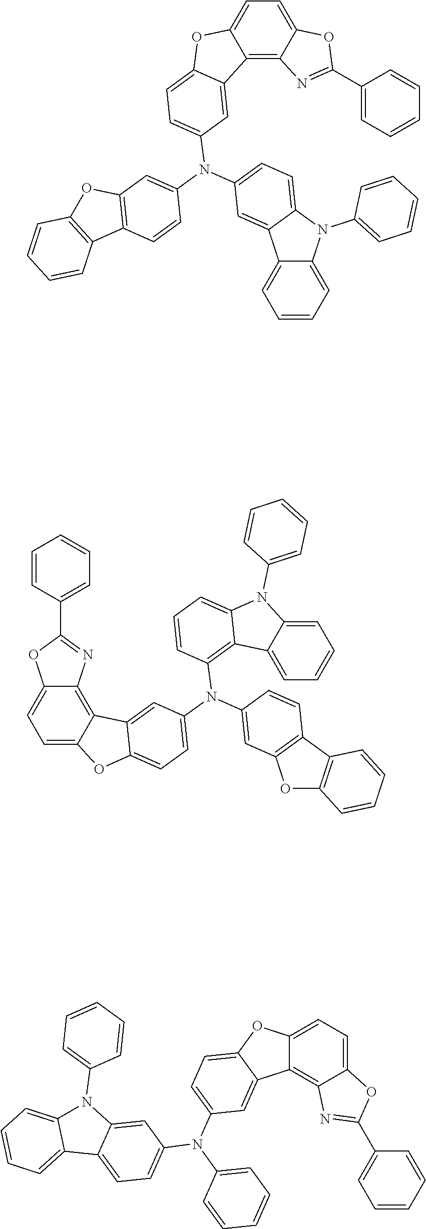 [SMILES: c1ccc(-c2nc3c(ccc4oc5ccc(N(c6ccc7c(c6)oc6ccccc67)c6ccc7c(c6)c6ccccc6n7-c6ccccc6)cc5c43)o2)cc1 ; c1ccc(-c2nc3c(ccc4oc5ccc(N(c6ccc7c(c6)oc6ccccc67)c6cccc7c6c6ccccc6n7-c6ccccc6)cc5c43)o2)cc1 ; c1ccc(-c2nc3c(ccc4oc5ccc(N(c6ccccc6)c6ccc7c8ccccc8n(-c8ccccc8)c7c6)cc5c43)o2)cc1]

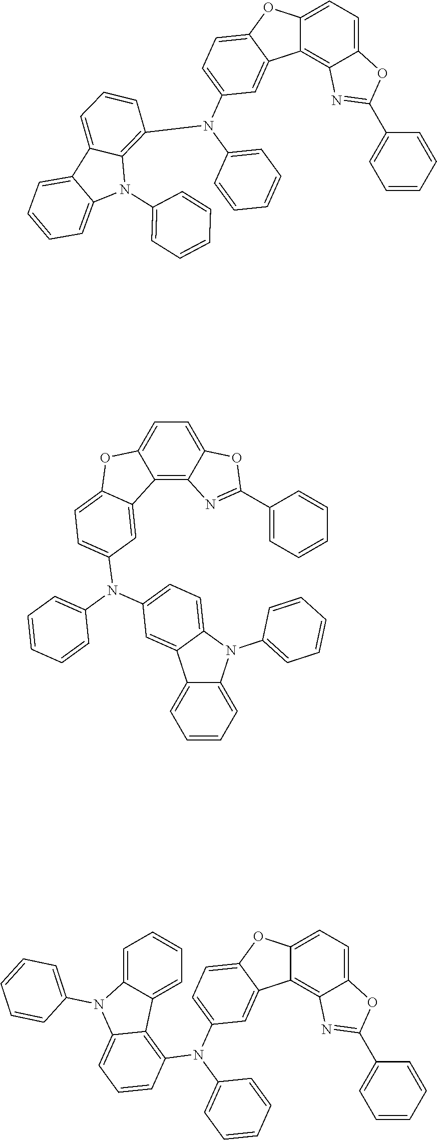 [SMILES: c1ccc(-c2nc3c(ccc4oc5ccc(N(c6ccccc6)c6cccc7c8ccccc8n(-c8ccccc8)c67)cc5c43)o2)cc1 ; c1ccc(-c2nc3c(ccc4oc5ccc(N(c6ccccc6)c6ccc7c(c6)c6ccccc6n7-c6ccccc6)cc5c43)o2)cc1 ; c1ccc(-c2nc3c(ccc4oc5ccc(N(c6ccccc6)c6cccc7c6c6ccccc6n7-c6ccccc6)cc5c43)o2)cc1]

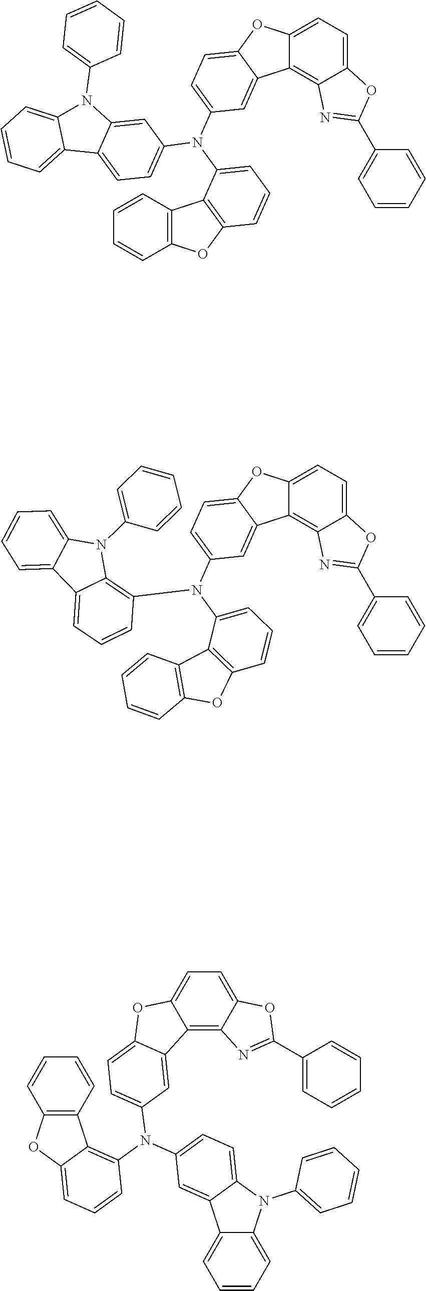 [SMILES: c1ccc(-c2nc3c(ccc4oc5ccc(N(c6ccc7c8ccccc8n(-c8ccccc8)c7c6)c6cccc7oc8ccccc8c67)cc5c43)o2)cc1 ; c1ccc(-c2nc3c(ccc4oc5ccc(N(c6cccc7oc8ccccc8c67)c6cccc7c8ccccc8n(-c8ccccc8)c67)cc5c43)o2)cc1 ; c1ccc(-c2nc3c(ccc4oc5ccc(N(c6ccc7c(c6)c6ccccc6n7-c6ccccc6)c6cccc7oc8ccccc8c67)cc5c43)o2)cc1]

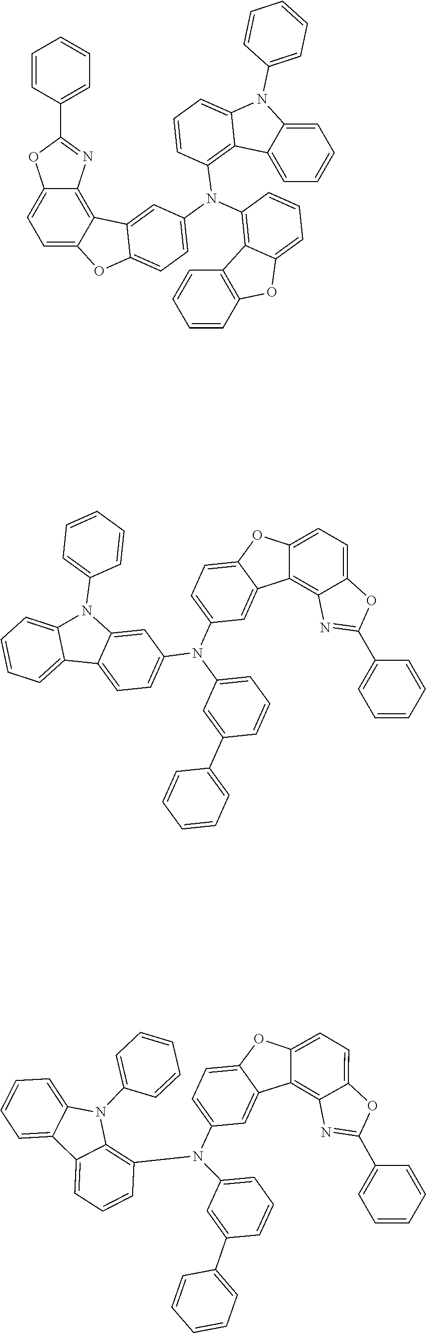 [SMILES: c1ccc(-c2nc3c(ccc4oc5ccc(N(c6cccc7oc8ccccc8c67)c6cccc7c6c6ccccc6n7-c6ccccc6)cc5c43)o2)cc1 ; c1ccc(-c2cccc(N(c3ccc4oc5ccc6oc(-c7ccccc7)nc6c5c4c3)c3ccc4c5ccccc5n(-c5ccccc5)c4c3)c2)cc1 ; c1ccc(-c2cccc(N(c3ccc4oc5ccc6oc(-c7ccccc7)nc6c5c4c3)c3cccc4c5ccccc5n(-c5ccccc5)c34)c2)cc1]

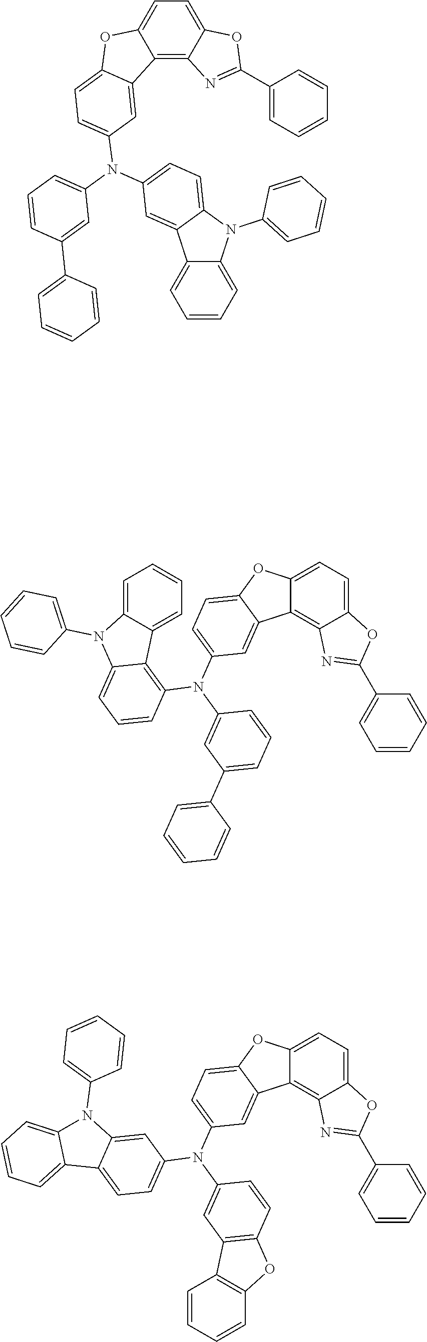 [SMILES: c1ccc(-c2cccc(N(c3ccc4oc5ccc6oc(-c7ccccc7)nc6c5c4c3)c3ccc4c(c3)c3ccccc3n4-c3ccccc3)c2)cc1 ; c1ccc(-c2cccc(N(c3ccc4oc5ccc6oc(-c7ccccc7)nc6c5c4c3)c3cccc4c3c3ccccc3n4-c3ccccc3)c2)cc1 ; c1ccc(-c2nc3c(ccc4oc5ccc(N(c6ccc7oc8ccccc8c7c6)c6ccc7c8ccccc8n(-c8ccccc8)c7c6)cc5c43)o2)cc1]

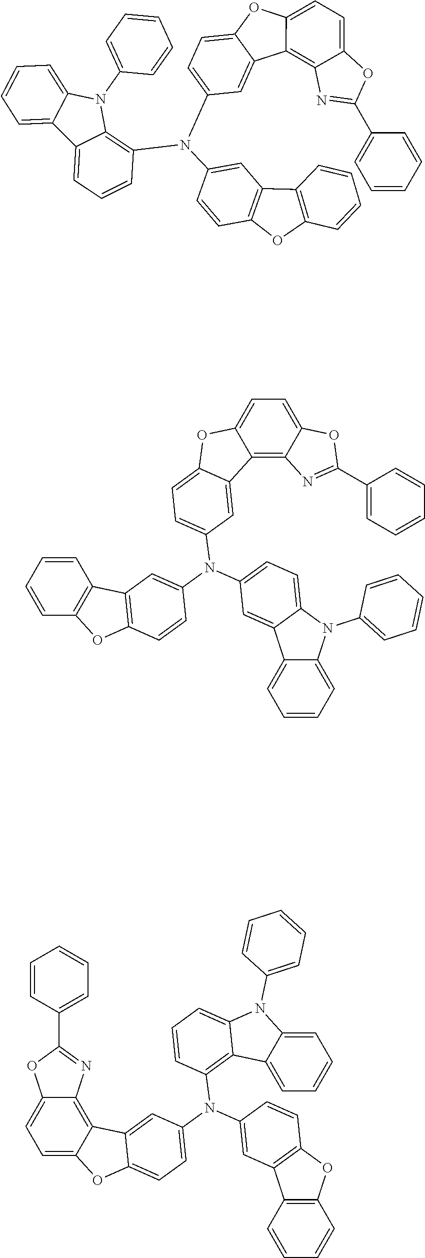 [SMILES: c1ccc(-c2nc3c(ccc4oc5ccc(N(c6ccc7oc8ccccc8c7c6)c6cccc7c8ccccc8n(-c8ccccc8)c67)cc5c43)o2)cc1 ; c1ccc(-c2nc3c(ccc4oc5ccc(N(c6ccc7oc8ccccc8c7c6)c6ccc7c(c6)c6ccccc6n7-c6ccccc6)cc5c43)o2)cc1 ; c1ccc(-c2nc3c(ccc4oc5ccc(N(c6ccc7oc8ccccc8c7c6)c6cccc7c6c6ccccc6n7-c6ccccc6)cc5c43)o2)cc1]

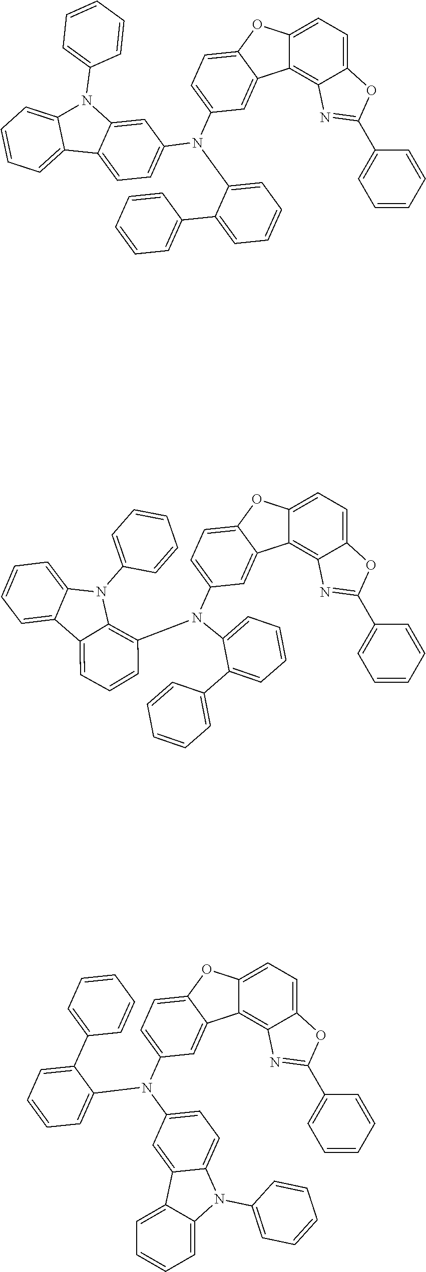 [SMILES: c1ccc(-c2nc3c(ccc4oc5ccc(N(c6ccc7c8ccccc8n(-c8ccccc8)c7c6)c6ccccc6-c6ccccc6)cc5c43)o2)cc1 ; c1ccc(-c2nc3c(ccc4oc5ccc(N(c6ccccc6-c6ccccc6)c6cccc7c8ccccc8n(-c8ccccc8)c67)cc5c43)o2)cc1 ; c1ccc(-c2nc3c(ccc4oc5ccc(N(c6ccc7c(c6)c6ccccc6n7-c6ccccc6)c6ccccc6-c6ccccc6)cc5c43)o2)cc1]

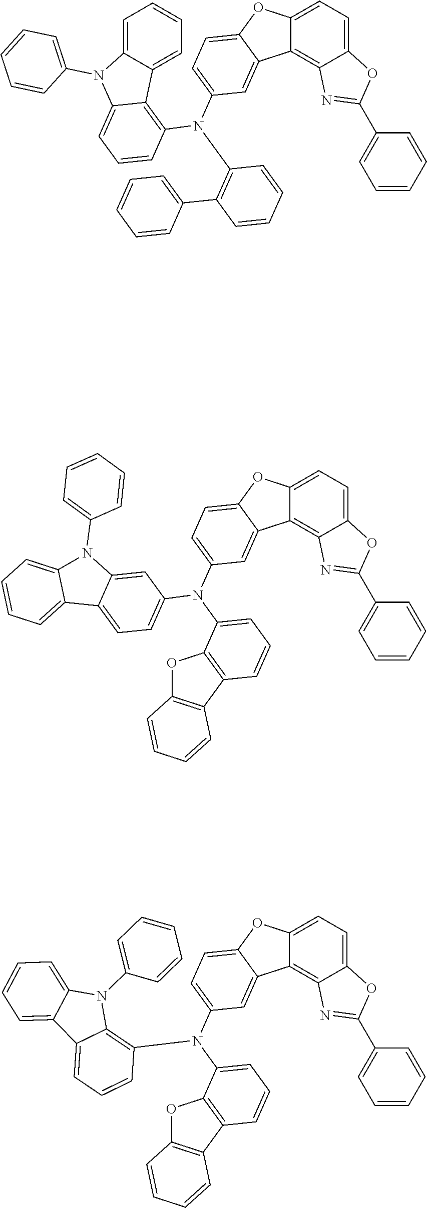 [SMILES: c1ccc(-c2nc3c(ccc4oc5ccc(N(c6ccccc6-c6ccccc6)c6cccc7c6c6ccccc6n7-c6ccccc6)cc5c43)o2)cc1 ; c1ccc(-c2nc3c(ccc4oc5ccc(N(c6ccc7c8ccccc8n(-c8ccccc8)c7c6)c6cccc7c6oc6ccccc67)cc5c43)o2)cc1 ; c1ccc(-c2nc3c(ccc4oc5ccc(N(c6cccc7c6oc6ccccc67)c6cccc7c8ccccc8n(-c8ccccc8)c67)cc5c43)o2)cc1]

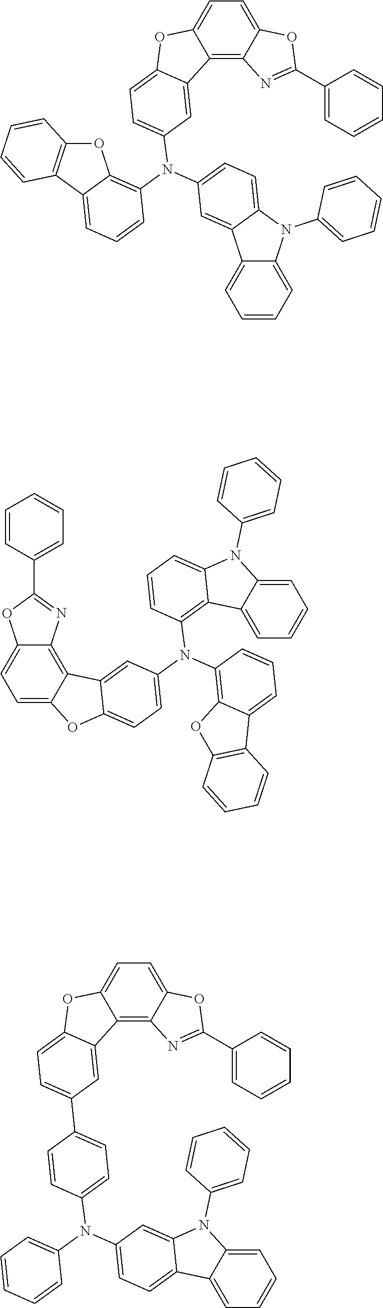 [SMILES: c1ccc(-c2nc3c(ccc4oc5ccc(N(c6ccc7c(c6)c6ccccc6n7-c6ccccc6)c6cccc7c6oc6ccccc67)cc5c43)o2)cc1 ; c1ccc(-c2nc3c(ccc4oc5ccc(N(c6cccc7c6oc6ccccc67)c6cccc7c6c6ccccc6n7-c6ccccc6)cc5c43)o2)cc1 ; c1ccc(-c2nc3c(ccc4oc5ccc(-c6ccc(N(c7ccccc7)c7ccc8c9ccccc9n(-c9ccccc9)c8c7)cc6)cc5c43)o2)cc1]

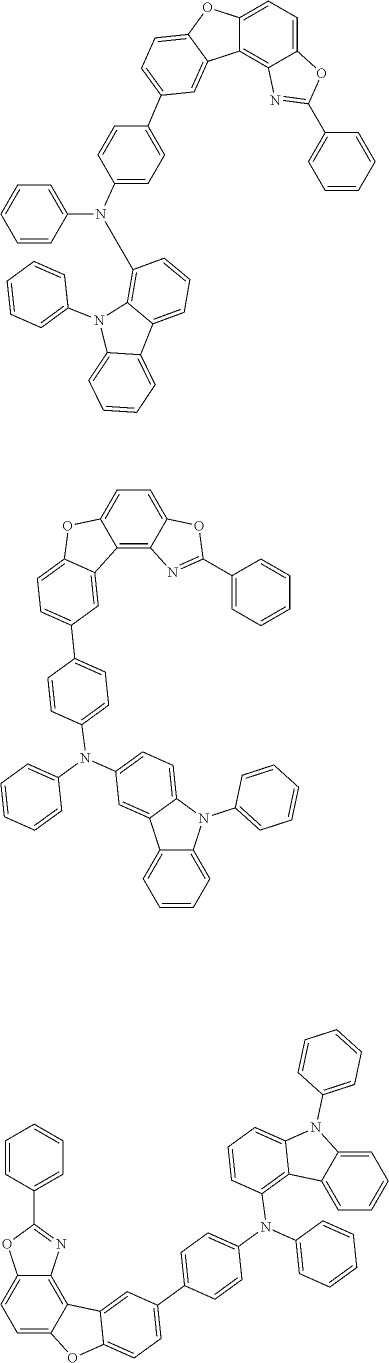 [SMILES: c1ccc(-c2nc3c(ccc4oc5ccc(-c6ccc(N(c7ccccc7)c7cccc8c9ccccc9n(-c9ccccc9)c78)cc6)cc5c43)o2)cc1 ; c1ccc(-c2nc3c(ccc4oc5ccc(-c6ccc(N(c7ccccc7)c7ccc8c(c7)c7ccccc7n8-c7ccccc7)cc6)cc5c43)o2)cc1 ; c1ccc(-c2nc3c(ccc4oc5ccc(-c6ccc(N(c7ccccc7)c7cccc8c7c7ccccc7n8-c7ccccc7)cc6)cc5c43)o2)cc1]

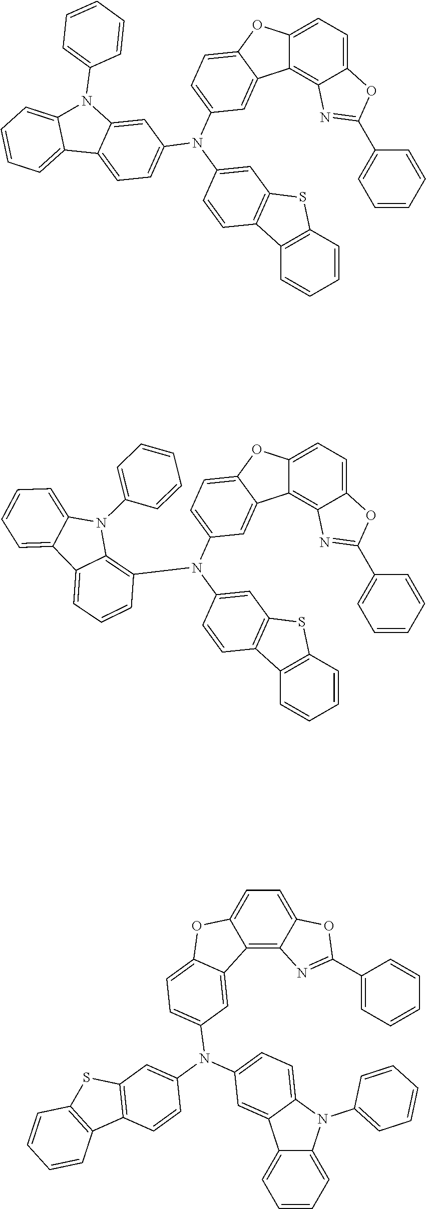 [SMILES: c1ccc(-c2nc3c(ccc4oc5ccc(N(c6ccc7c(c6)sc6ccccc67)c6ccc7c8ccccc8n(-c8ccccc8)c7c6)cc5c43)o2)cc1 ; c1ccc(-c2nc3c(ccc4oc5ccc(N(c6ccc7c(c6)sc6ccccc67)c6cccc7c8ccccc8n(-c8ccccc8)c67)cc5c43)o2)cc1 ; c1ccc(-c2nc3c(ccc4oc5ccc(N(c6ccc7c(c6)sc6ccccc67)c6ccc7c(c6)c6ccccc6n7-c6ccccc6)cc5c43)o2)cc1]

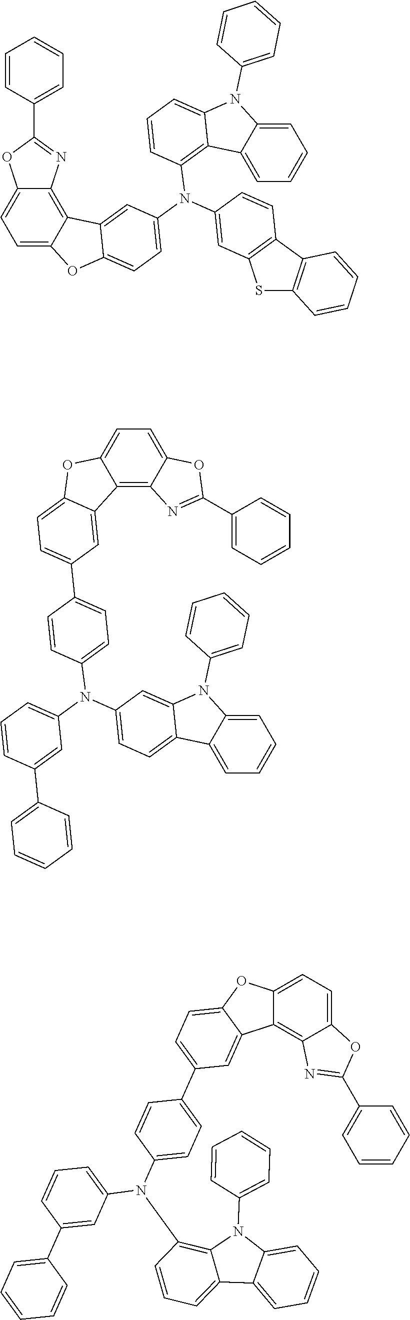 [SMILES: c1ccc(-c2nc3c(ccc4oc5ccc(N(c6ccc7c(c6)sc6ccccc67)c6cccc7c6c6ccccc6n7-c6ccccc6)cc5c43)o2)cc1 ; c1ccc(-c2cccc(N(c3ccc(-c4ccc5oc6ccc7oc(-c8ccccc8)nc7c6c5c4)cc3)c3ccc4c5ccccc5n(-c5ccccc5)c4c3)c2)cc1 ; c1ccc(-c2cccc(N(c3ccc(-c4ccc5oc6ccc7oc(-c8ccccc8)nc7c6c5c4)cc3)c3cccc4c5ccccc5n(-c5ccccc5)c34)c2)cc1]

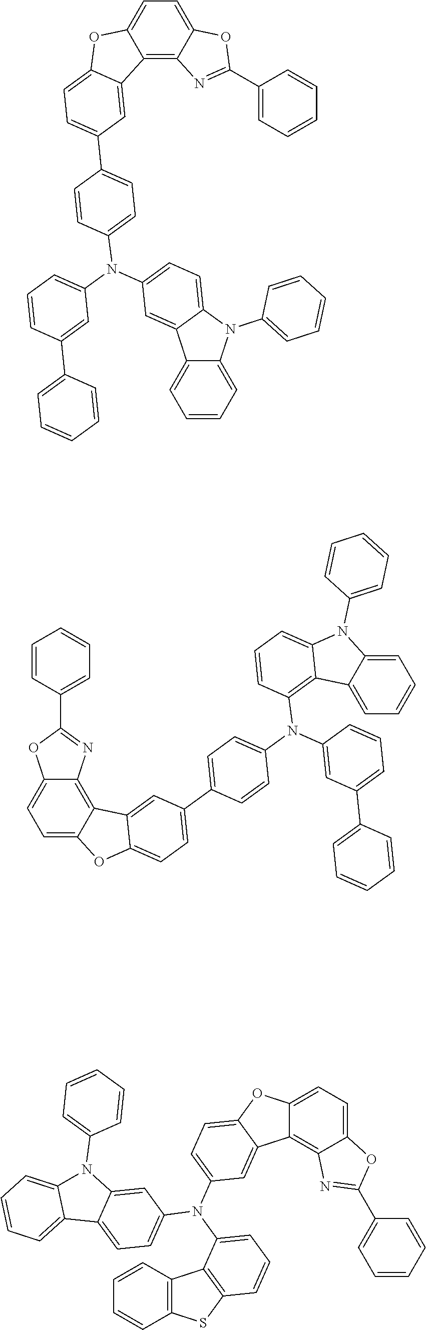 [SMILES: c1ccc(-c2cccc(N(c3ccc(-c4ccc5oc6ccc7oc(-c8ccccc8)nc7c6c5c4)cc3)c3ccc4c(c3)c3ccccc3n4-c3ccccc3)c2)cc1 ; c1ccc(-c2cccc(N(c3ccc(-c4ccc5oc6ccc7oc(-c8ccccc8)nc7c6c5c4)cc3)c3cccc4c3c3ccccc3n4-c3ccccc3)c2)cc1 ; c1ccc(-c2nc3c(ccc4oc5ccc(N(c6ccc7c8ccccc8n(-c8ccccc8)c7c6)c6cccc7sc8ccccc8c67)cc5c43)o2)cc1]

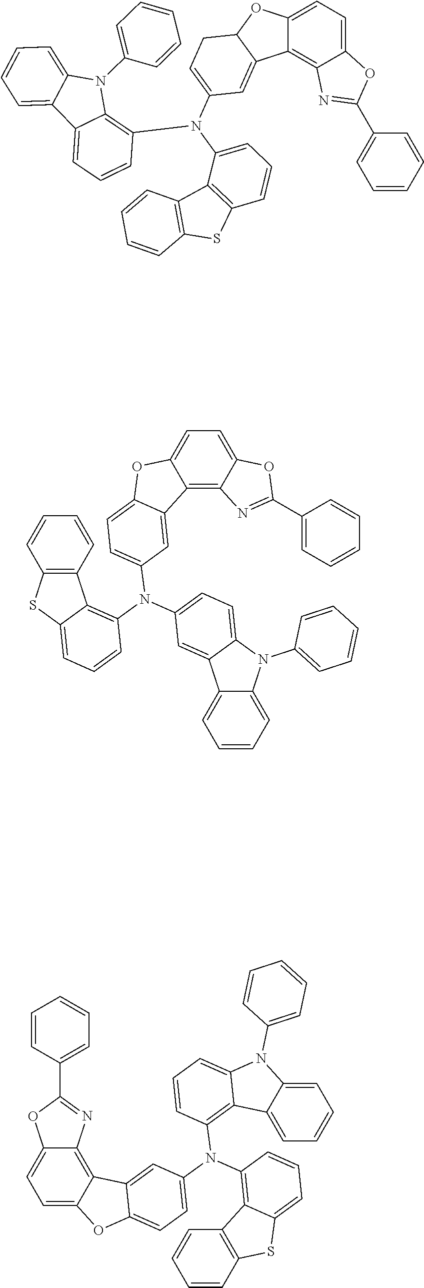 [SMILES: C1=C2c3c(ccc4oc(-c5ccccc5)nc34)OC2CC=C1N(c1cccc2sc3ccccc3c12)c1cccc2c3ccccc3n(-c3ccccc3)c12 ; c1ccc(-c2nc3c(ccc4oc5ccc(N(c6ccc7c(c6)c6ccccc6n7-c6ccccc6)c6cccc7sc8ccccc8c67)cc5c43)o2)cc1 ; c1ccc(-c2nc3c(ccc4oc5ccc(N(c6cccc7sc8ccccc8c67)c6cccc7c6c6ccccc6n7-c6ccccc6)cc5c43)o2)cc1]

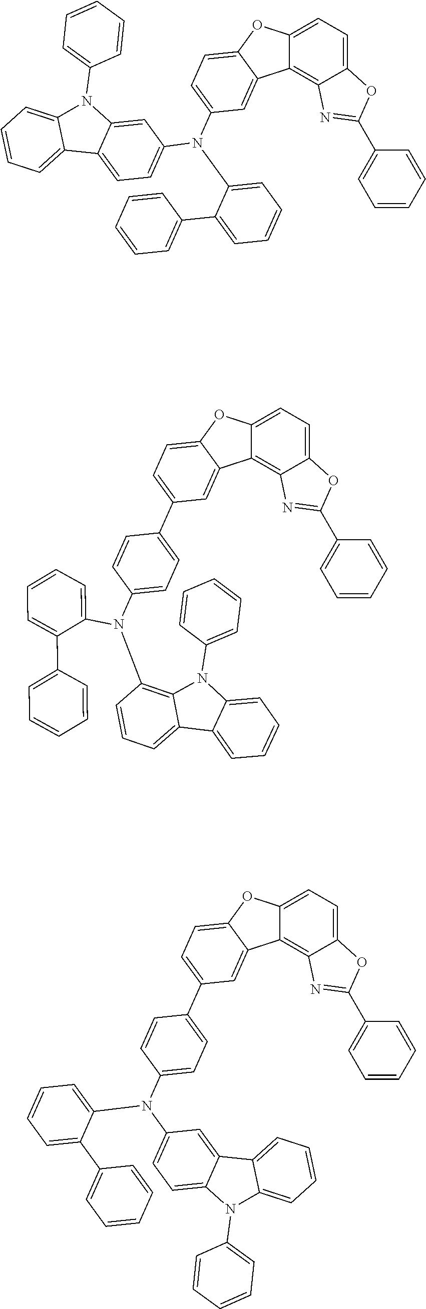 [SMILES: c1ccc(-c2nc3c(ccc4oc5ccc(N(c6ccc7c8ccccc8n(-c8ccccc8)c7c6)c6ccccc6-c6ccccc6)cc5c43)o2)cc1 ; c1ccc(-c2nc3c(ccc4oc5ccc(-c6ccc(N(c7ccccc7-c7ccccc7)c7cccc8c9ccccc9n(-c9ccccc9)c78)cc6)cc5c43)o2)cc1 ; c1ccc(-c2nc3c(ccc4oc5ccc(-c6ccc(N(c7ccc8c(c7)c7ccccc7n8-c7ccccc7)c7ccccc7-c7ccccc7)cc6)cc5c43)o2)cc1]

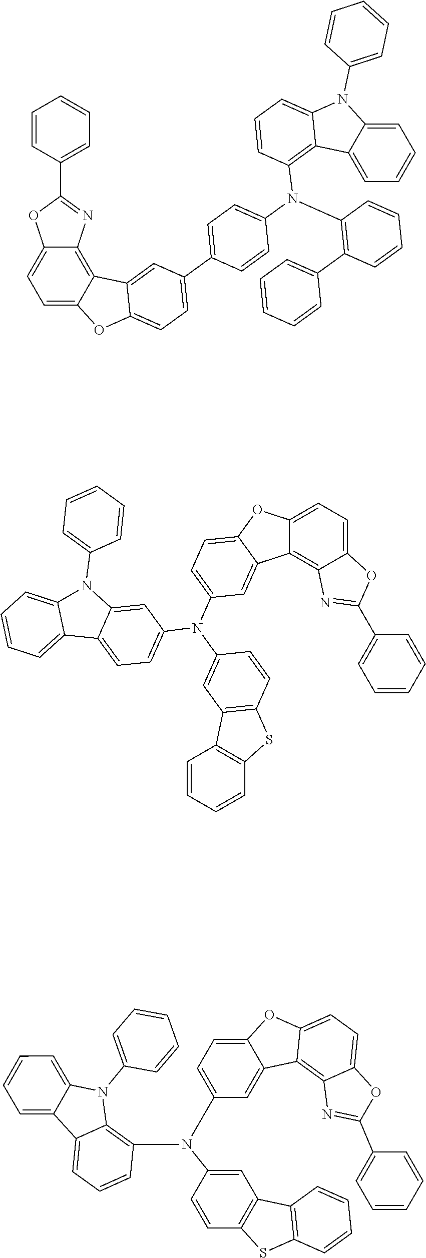 [SMILES: c1ccc(-c2nc3c(ccc4oc5ccc(-c6ccc(N(c7ccccc7-c7ccccc7)c7cccc8c7c7ccccc7n8-c7ccccc7)cc6)cc5c43)o2)cc1 ; c1ccc(-c2nc3c(ccc4oc5ccc(N(c6ccc7sc8ccccc8c7c6)c6ccc7c8ccccc8n(-c8ccccc8)c7c6)cc5c43)o2)cc1 ; c1ccc(-c2nc3c(ccc4oc5ccc(N(c6ccc7sc8ccccc8c7c6)c6cccc7c8ccccc8n(-c8ccccc8)c67)cc5c43)o2)cc1]

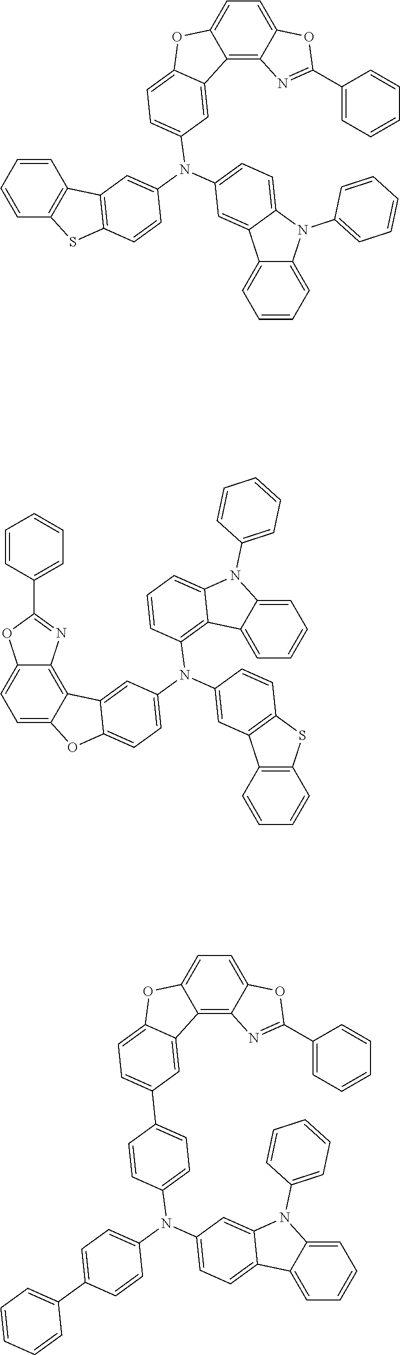 [SMILES: c1ccc(-c2nc3c(ccc4oc5ccc(N(c6ccc7sc8ccccc8c7c6)c6ccc7c(c6)c6ccccc6n7-c6ccccc6)cc5c43)o2)cc1 ; c1ccc(-c2nc3c(ccc4oc5ccc(N(c6ccc7sc8ccccc8c7c6)c6cccc7c6c6ccccc6n7-c6ccccc6)cc5c43)o2)cc1 ; c1ccc(-c2ccc(N(c3ccc(-c4ccc5oc6ccc7oc(-c8ccccc8)nc7c6c5c4)cc3)c3ccc4c5ccccc5n(-c5ccccc5)c4c3)cc2)cc1]

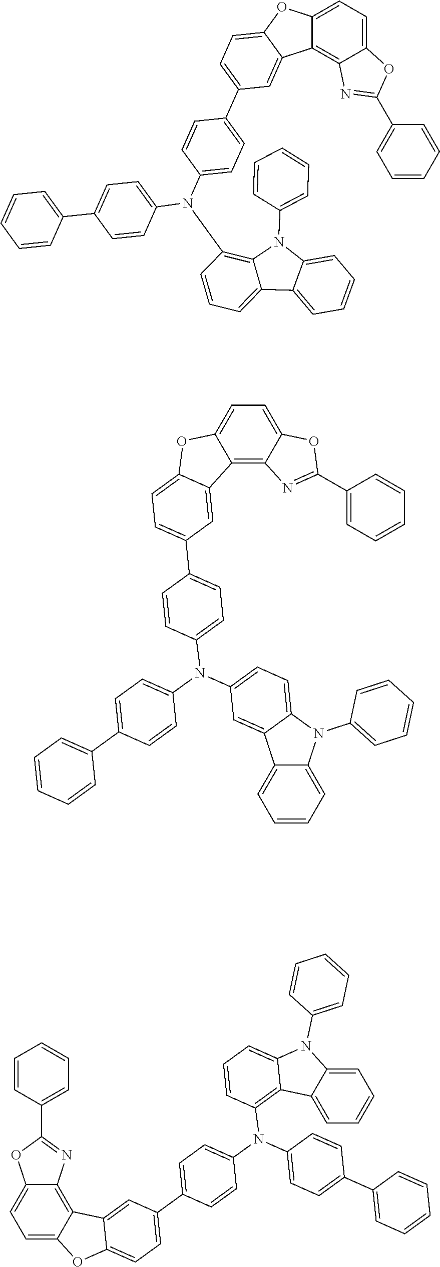 [SMILES: c1ccc(-c2ccc(N(c3ccc(-c4ccc5oc6ccc7oc(-c8ccccc8)nc7c6c5c4)cc3)c3cccc4c5ccccc5n(-c5ccccc5)c34)cc2)cc1 ; c1ccc(-c2ccc(N(c3ccc(-c4ccc5oc6ccc7oc(-c8ccccc8)nc7c6c5c4)cc3)c3ccc4c(c3)c3ccccc3n4-c3ccccc3)cc2)cc1 ; c1ccc(-c2ccc(N(c3ccc(-c4ccc5oc6ccc7oc(-c8ccccc8)nc7c6c5c4)cc3)c3cccc4c3c3ccccc3n4-c3ccccc3)cc2)cc1]

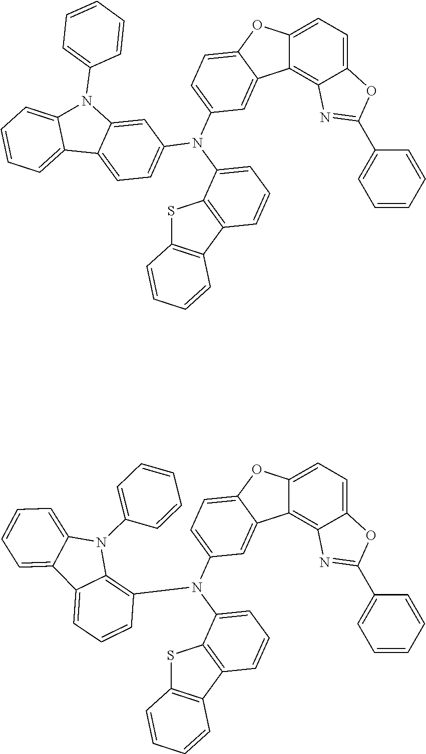 [SMILES: c1ccc(-c2nc3c(ccc4oc5ccc(N(c6ccc7c8ccccc8n(-c8ccccc8)c7c6)c6cccc7c6sc6ccccc67)cc5c43)o2)cc1 ; c1ccc(-c2nc3c(ccc4oc5ccc(N(c6cccc7c6sc6ccccc67)c6cccc7c8ccccc8n(-c8ccccc8)c67)cc5c43)o2)cc1]

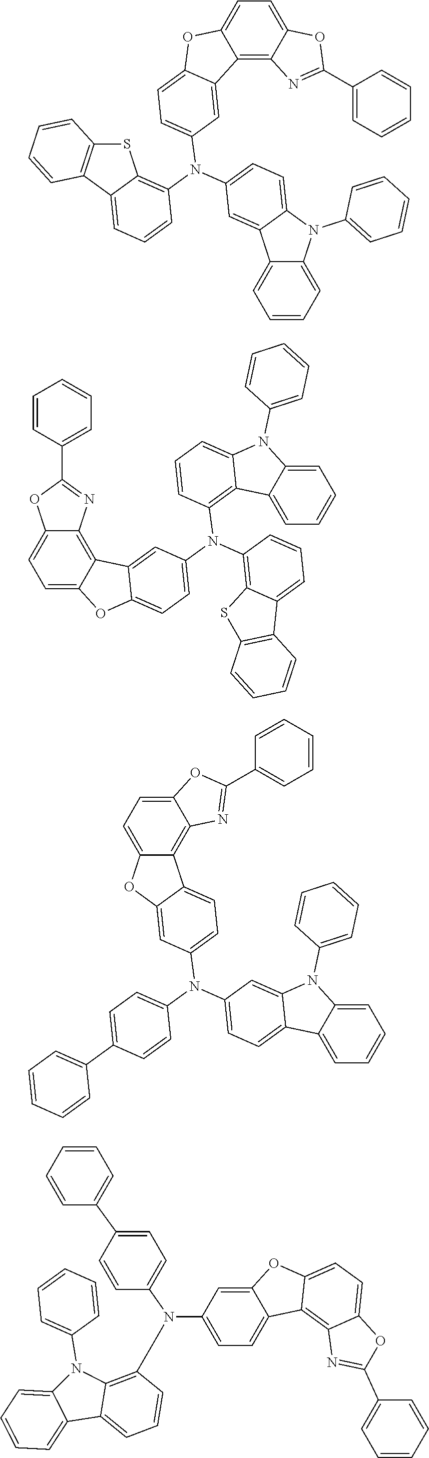

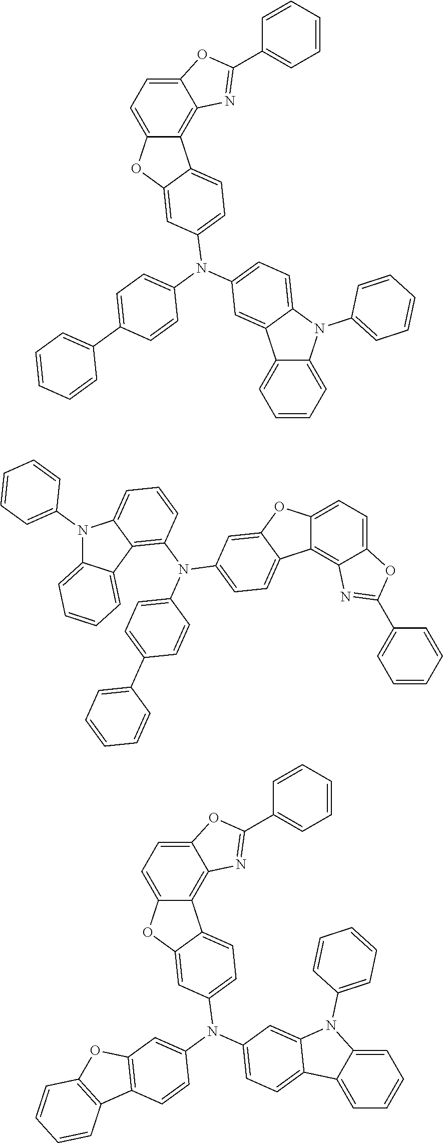 [SMILES: c1ccc(-c2ccc(N(c3ccc4c(c3)oc3ccc5oc(-c6ccccc6)nc5c34)c3ccc4c(c3)c3ccccc3n4-c3ccccc3)cc2)cc1 ; c1ccc(-c2ccc(N(c3ccc4c(c3)oc3ccc5oc(-c6ccccc6)nc5c34)c3cccc4c3c3ccccc3n4-c3ccccc3)cc2)cc1 ; c1ccc(-c2nc3c(ccc4oc5cc(N(c6ccc7c(c6)oc6ccccc67)c6ccc7c8ccccc8n(-c8ccccc8)c7c6)ccc5c43)o2)cc1]

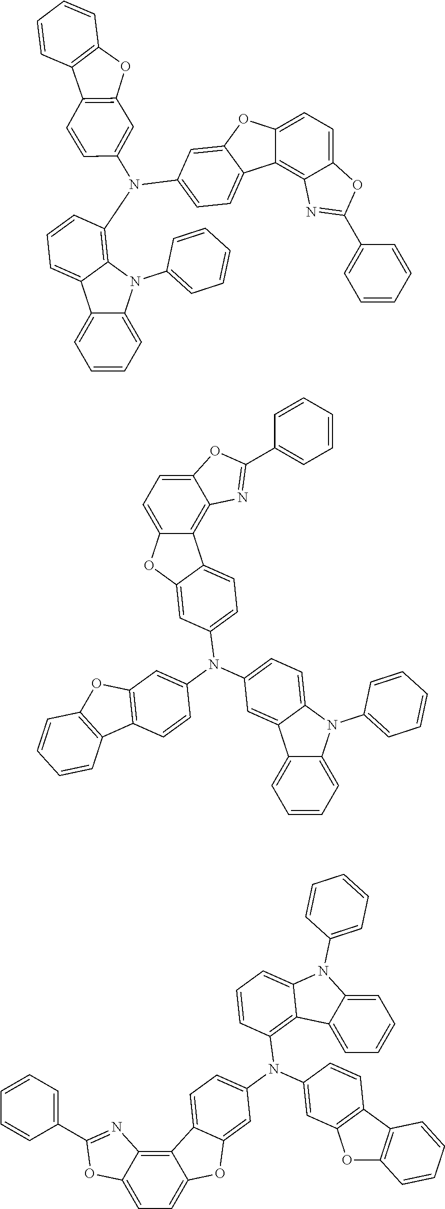 [SMILES: c1ccc(-c2nc3c(ccc4oc5cc(N(c6ccc7c(c6)oc6ccccc67)c6cccc7c8ccccc8n(-c8ccccc8)c67)ccc5c43)o2)cc1 ; c1ccc(-c2nc3c(ccc4oc5cc(N(c6ccc7c(c6)oc6ccccc67)c6ccc7c(c6)c6ccccc6n7-c6ccccc6)ccc5c43)o2)cc1 ; c1ccc(-c2nc3c(ccc4oc5cc(N(c6ccc7c(c6)oc6ccccc67)c6cccc7c6c6ccccc6n7-c6ccccc6)ccc5c43)o2)cc1]

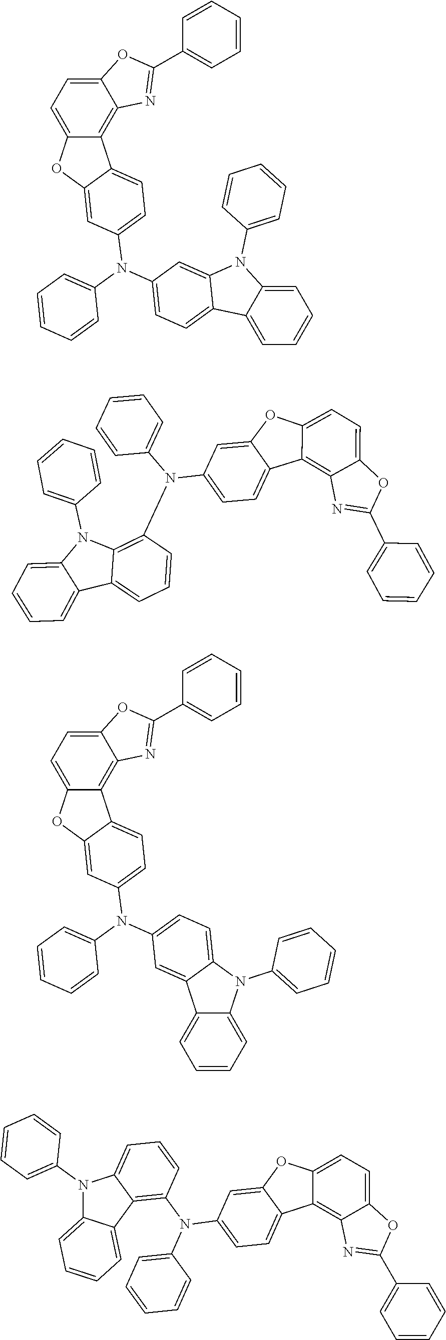 [SMILES: c1ccc(-c2nc3c(ccc4oc5cc(N(c6ccccc6)c6ccc7c8ccccc8n(-c8ccccc8)c7c6)ccc5c43)o2)cc1 ; c1ccc(-c2nc3c(ccc4oc5cc(N(c6ccccc6)c6cccc7c8ccccc8n(-c8ccccc8)c67)ccc5c43)o2)cc1 ; c1ccc(-c2nc3c(ccc4oc5cc(N(c6ccccc6)c6ccc7c(c6)c6ccccc6n7-c6ccccc6)ccc5c43)o2)cc1 ; c1ccc(-c2nc3c(ccc4oc5cc(N(c6ccccc6)c6cccc7c6c6ccccc6n7-c6ccccc6)ccc5c43)o2)cc1]

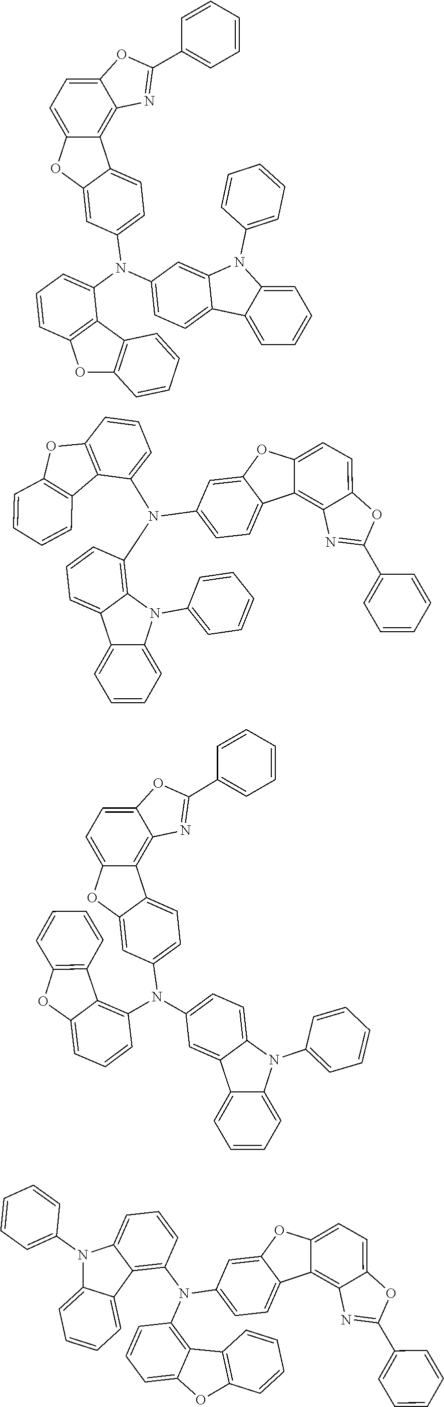

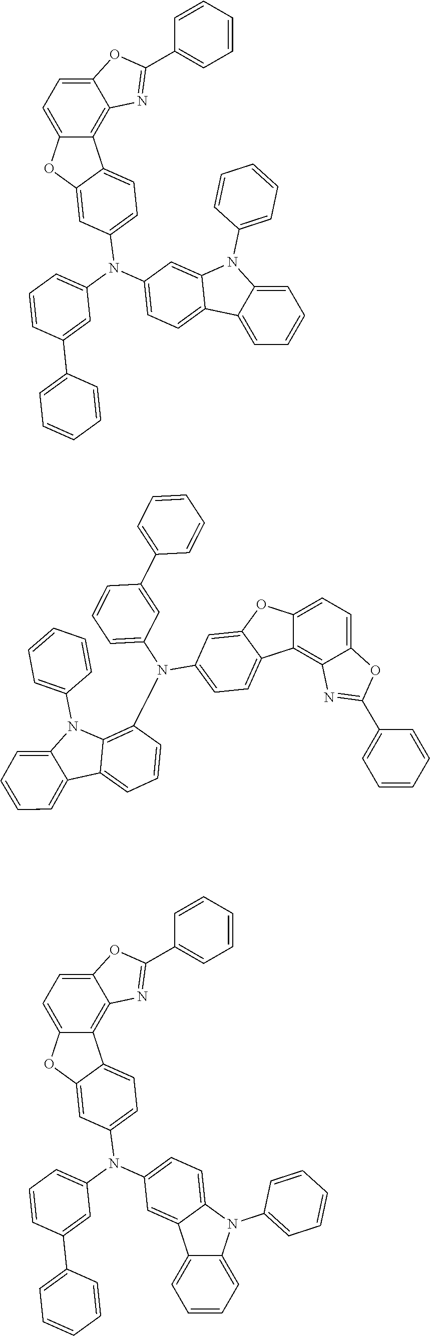 [SMILES: c1ccc(-c2cccc(N(c3ccc4c(c3)oc3ccc5oc(-c6ccccc6)nc5c34)c3ccc4c5ccccc5n(-c5ccccc5)c4c3)c2)cc1 ; c1ccc(-c2cccc(N(c3ccc4c(c3)oc3ccc5oc(-c6ccccc6)nc5c34)c3cccc4c5ccccc5n(-c5ccccc5)c34)c2)cc1 ; c1ccc(-c2cccc(N(c3ccc4c(c3)oc3ccc5oc(-c6ccccc6)nc5c34)c3ccc4c(c3)c3ccccc3n4-c3ccccc3)c2)cc1]

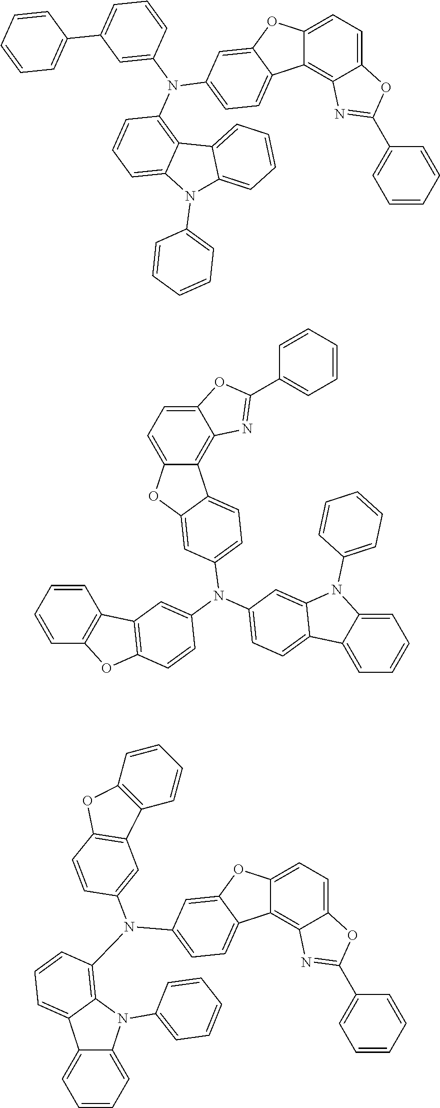 [SMILES: c1ccc(-c2cccc(N(c3ccc4c(c3)oc3ccc5oc(-c6ccccc6)nc5c34)c3cccc4c3c3ccccc3n4-c3ccccc3)c2)cc1 ; c1ccc(-c2nc3c(ccc4oc5cc(N(c6ccc7oc8ccccc8c7c6)c6ccc7c8ccccc8n(-c8ccccc8)c7c6)ccc5c43)o2)cc1 ; c1ccc(-c2nc3c(ccc4oc5cc(N(c6ccc7oc8ccccc8c7c6)c6cccc7c8ccccc8n(-c8ccccc8)c67)ccc5c43)o2)cc1]

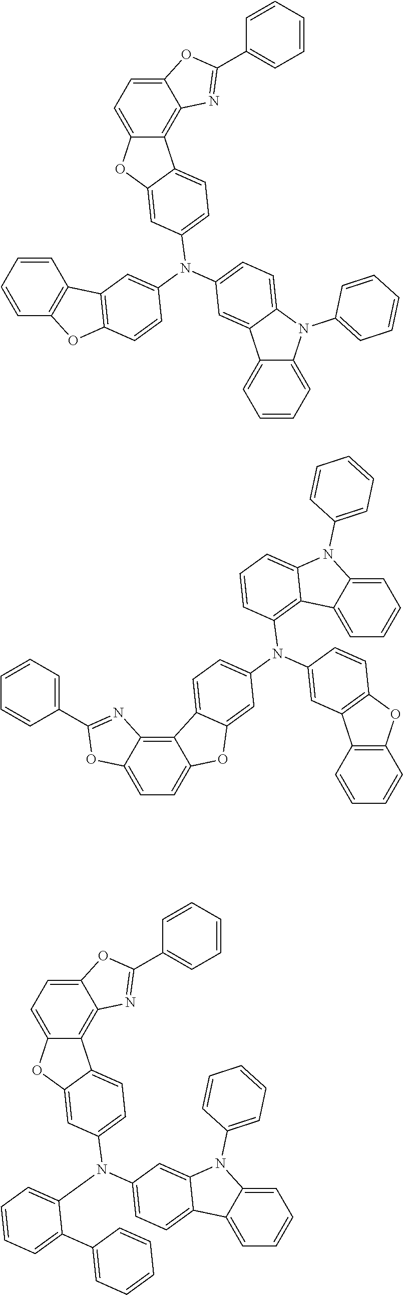 [SMILES: c1ccc(-c2nc3c(ccc4oc5cc(N(c6ccc7oc8ccccc8c7c6)c6ccc7c(c6)c6ccccc6n7-c6ccccc6)ccc5c43)o2)cc1 ; c1ccc(-c2nc3c(ccc4oc5cc(N(c6ccc7oc8ccccc8c7c6)c6cccc7c6c6ccccc6n7-c6ccccc6)ccc5c43)o2)cc1 ; c1ccc(-c2nc3c(ccc4oc5cc(N(c6ccc7c8ccccc8n(-c8ccccc8)c7c6)c6ccccc6-c6ccccc6)ccc5c43)o2)cc1]

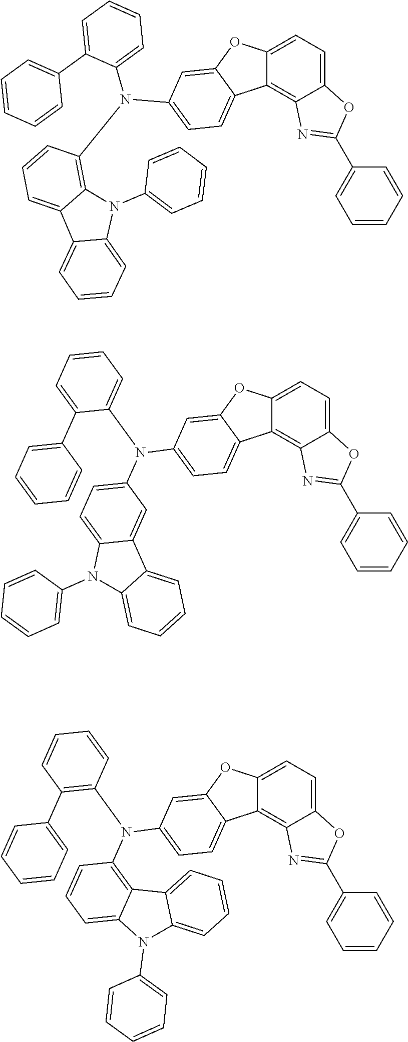 [SMILES: c1ccc(-c2nc3c(ccc4oc5cc(N(c6ccccc6-c6ccccc6)c6cccc7c8ccccc8n(-c8ccccc8)c67)ccc5c43)o2)cc1 ; c1ccc(-c2nc3c(ccc4oc5cc(N(c6ccc7c(c6)c6ccccc6n7-c6ccccc6)c6ccccc6-c6ccccc6)ccc5c43)o2)cc1 ; c1ccc(-c2nc3c(ccc4oc5cc(N(c6ccccc6-c6ccccc6)c6cccc7c6c6ccccc6n7-c6ccccc6)ccc5c43)o2)cc1]

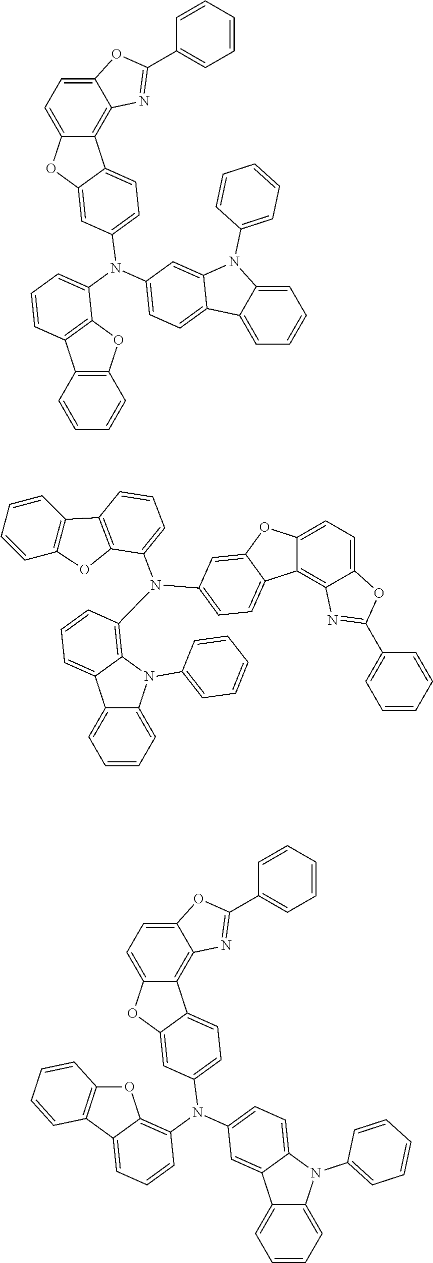 [SMILES: c1ccc(-c2nc3c(ccc4oc5cc(N(c6ccc7c8ccccc8n(-c8ccccc8)c7c6)c6cccc7c6oc6ccccc67)ccc5c43)o2)cc1 ; c1ccc(-c2nc3c(ccc4oc5cc(N(c6cccc7c6oc6ccccc67)c6cccc7c8ccccc8n(-c8ccccc8)c67)ccc5c43)o2)cc1 ; c1ccc(-c2nc3c(ccc4oc5cc(N(c6ccc7c(c6)c6ccccc6n7-c6ccccc6)c6cccc7c6oc6ccccc67)ccc5c43)o2)cc1]

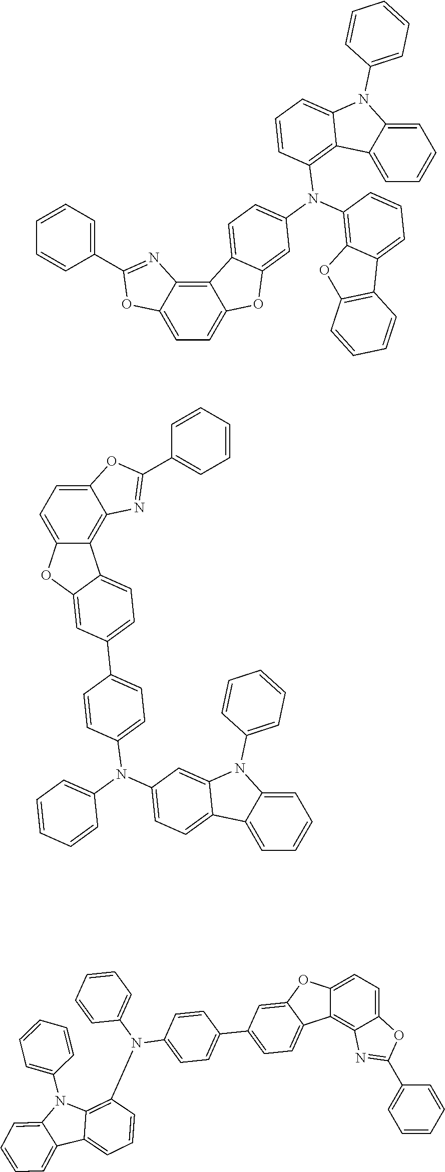 [SMILES: c1ccc(-c2nc3c(ccc4oc5cc(N(c6cccc7c6oc6ccccc67)c6cccc7c6c6ccccc6n7-c6ccccc6)ccc5c43)o2)cc1 ; c1ccc(-c2nc3c(ccc4oc5cc(-c6ccc(N(c7ccccc7)c7ccc8c9ccccc9n(-c9ccccc9)c8c7)cc6)ccc5c43)o2)cc1 ; c1ccc(-c2nc3c(ccc4oc5cc(-c6ccc(N(c7ccccc7)c7cccc8c9ccccc9n(-c9ccccc9)c78)cc6)ccc5c43)o2)cc1]

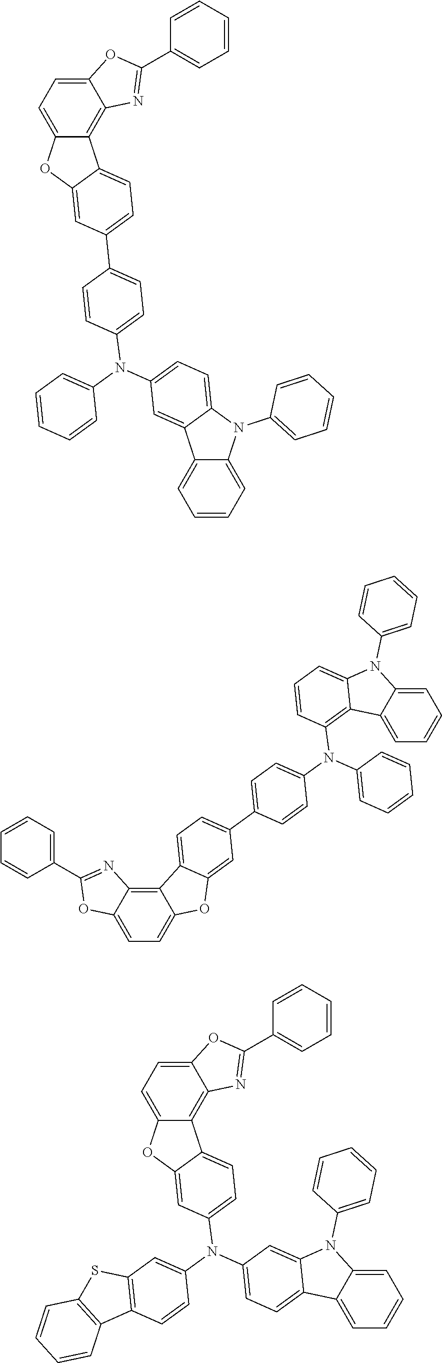 [SMILES: c1ccc(-c2nc3c(ccc4oc5cc(-c6ccc(N(c7ccccc7)c7ccc8c(c7)c7ccccc7n8-c7ccccc7)cc6)ccc5c43)o2)cc1 ; c1ccc(-c2nc3c(ccc4oc5cc(-c6ccc(N(c7ccccc7)c7cccc8c7c7ccccc7n8-c7ccccc7)cc6)ccc5c43)o2)cc1 ; c1ccc(-c2nc3c(ccc4oc5cc(N(c6ccc7c(c6)sc6ccccc67)c6ccc7c8ccccc8n(-c8ccccc8)c7c6)ccc5c43)o2)cc1]

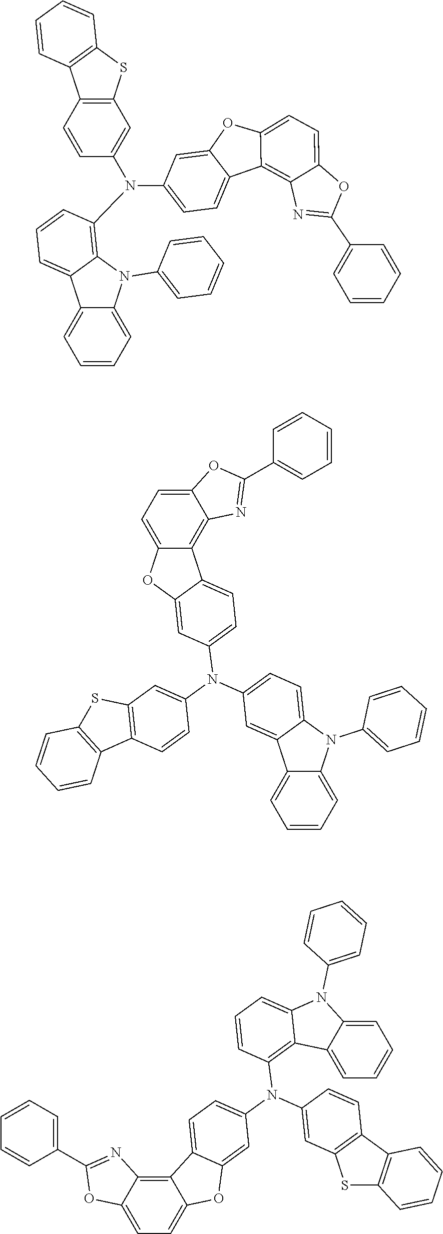 [SMILES: c1ccc(-c2nc3c(ccc4oc5cc(N(c6ccc7c(c6)sc6ccccc67)c6cccc7c8ccccc8n(-c8ccccc8)c67)ccc5c43)o2)cc1 ; c1ccc(-c2nc3c(ccc4oc5cc(N(c6ccc7c(c6)sc6ccccc67)c6ccc7c(c6)c6ccccc6n7-c6ccccc6)ccc5c43)o2)cc1 ; c1ccc(-c2nc3c(ccc4oc5cc(N(c6ccc7c(c6)sc6ccccc67)c6cccc7c6c6ccccc6n7-c6ccccc6)ccc5c43)o2)cc1]

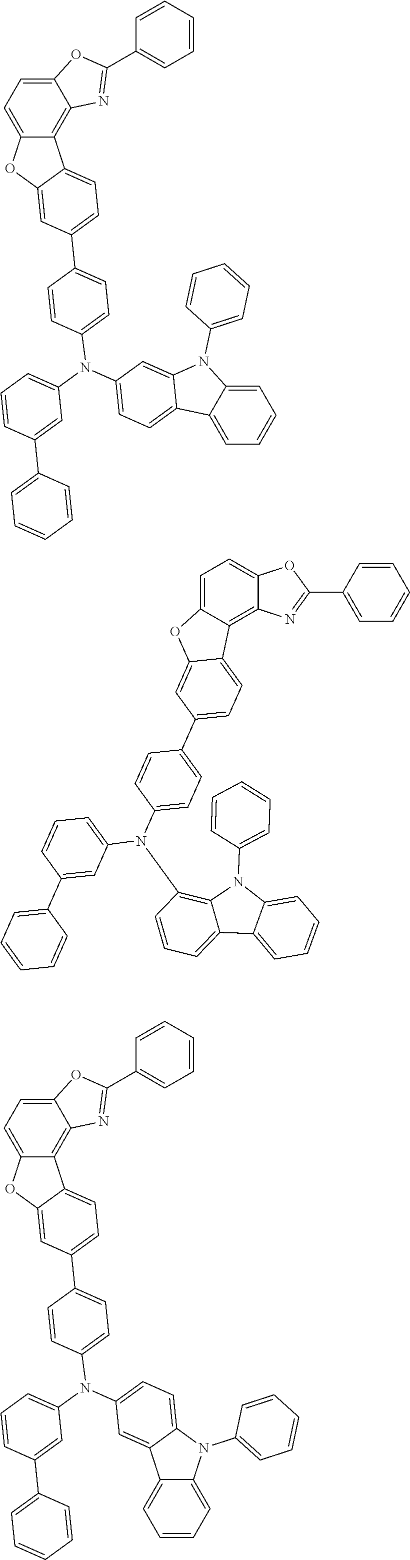 [SMILES: c1ccc(-c2cccc(N(c3ccc(-c4ccc5c(c4)oc4ccc6oc(-c7ccccc7)nc6c45)cc3)c3ccc4c5ccccc5n(-c5ccccc5)c4c3)c2)cc1 ; c1ccc(-c2cccc(N(c3ccc(-c4ccc5c(c4)oc4ccc6oc(-c7ccccc7)nc6c45)cc3)c3cccc4c5ccccc5n(-c5ccccc5)c34)c2)cc1 ; c1ccc(-c2cccc(N(c3ccc(-c4ccc5c(c4)oc4ccc6oc(-c7ccccc7)nc6c45)cc3)c3ccc4c(c3)c3ccccc3n4-c3ccccc3)c2)cc1]

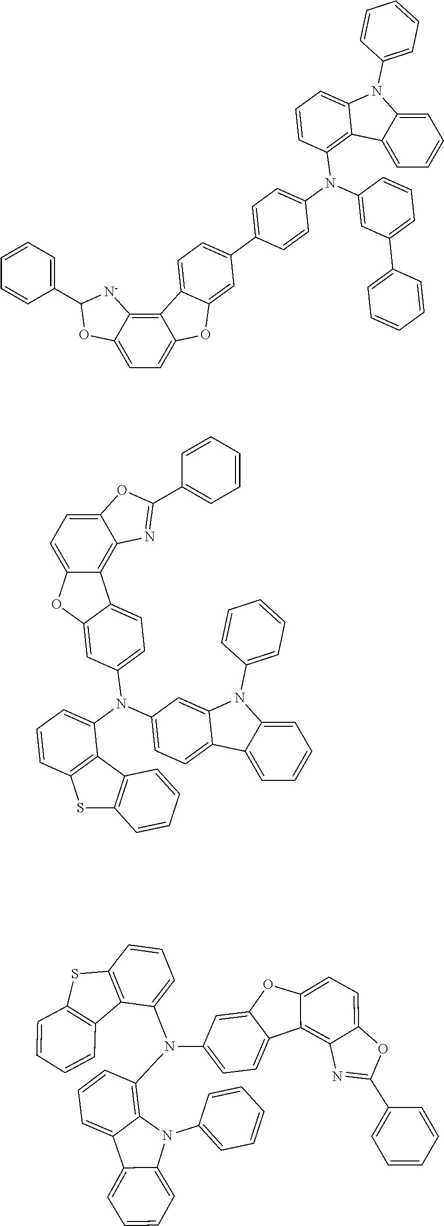 [SMILES: c1ccc(-c2cccc(N(c3ccc(-c4ccc5c(c4)oc4ccc6c(c45)[N-]C(c4ccccc4)O6)cc3)c3cccc4c3c3ccccc3n4-c3ccccc3)c2)cc1 ; c1ccc(-c2nc3c(ccc4oc5cc(N(c6ccc7c8ccccc8n(-c8ccccc8)c7c6)c6cccc7sc8ccccc8c67)ccc5c43)o2)cc1 ; c1ccc(-c2nc3c(ccc4oc5cc(N(c6cccc7sc8ccccc8c67)c6cccc7c8ccccc8n(-c8ccccc8)c67)ccc5c43)o2)cc1]

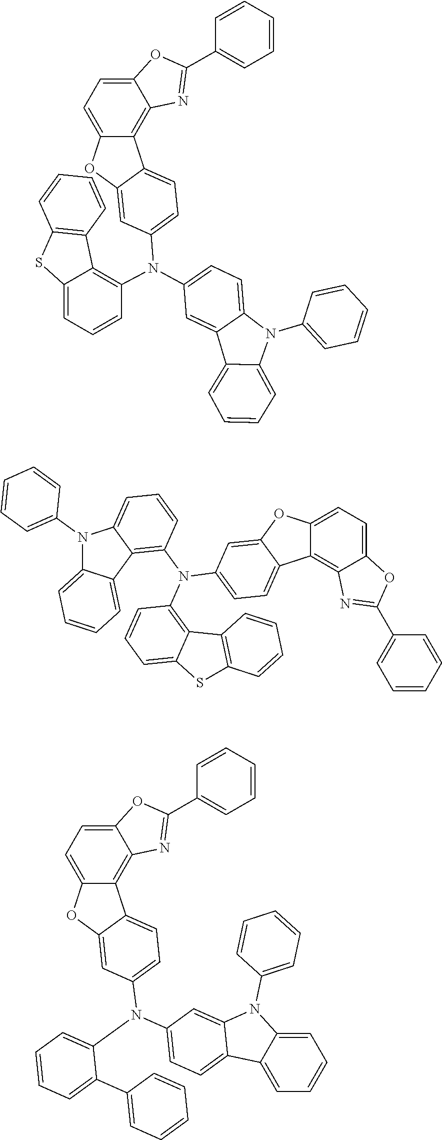 [SMILES: c1ccc(-c2nc3c(ccc4oc5cc(N(c6ccc7c(c6)c6ccccc6n7-c6ccccc6)c6cccc7sc8ccccc8c67)ccc5c43)o2)cc1 ; c1ccc(-c2nc3c(ccc4oc5cc(N(c6cccc7sc8ccccc8c67)c6cccc7c6c6ccccc6n7-c6ccccc6)ccc5c43)o2)cc1 ; c1ccc(-c2nc3c(ccc4oc5cc(N(c6ccc7c8ccccc8n(-c8ccccc8)c7c6)c6ccccc6-c6ccccc6)ccc5c43)o2)cc1]

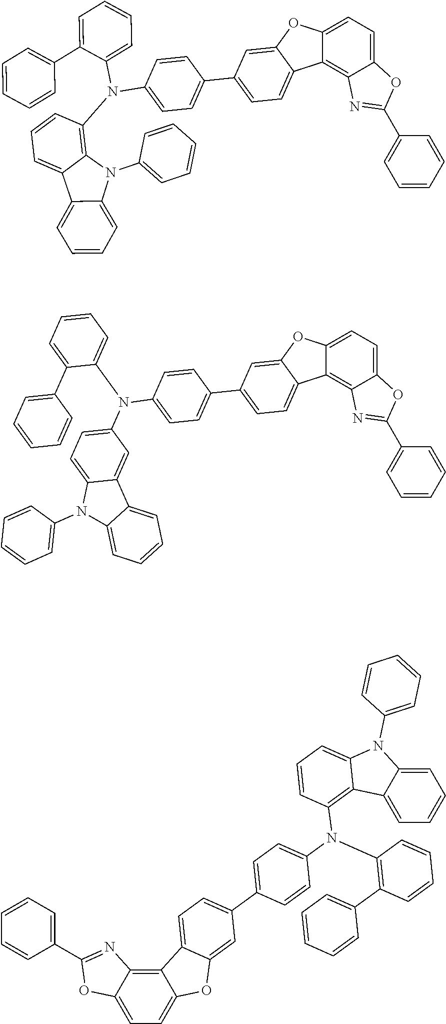 [SMILES: c1ccc(-c2nc3c(ccc4oc5cc(-c6ccc(N(c7ccccc7-c7ccccc7)c7cccc8c9ccccc9n(-c9ccccc9)c78)cc6)ccc5c43)o2)cc1 ; c1ccc(-c2nc3c(ccc4oc5cc(-c6ccc(N(c7ccc8c(c7)c7ccccc7n8-c7ccccc7)c7ccccc7-c7ccccc7)cc6)ccc5c43)o2)cc1 ; c1ccc(-c2nc3c(ccc4oc5cc(-c6ccc(N(c7ccccc7-c7ccccc7)c7cccc8c7c7ccccc7n8-c7ccccc7)cc6)ccc5c43)o2)cc1]

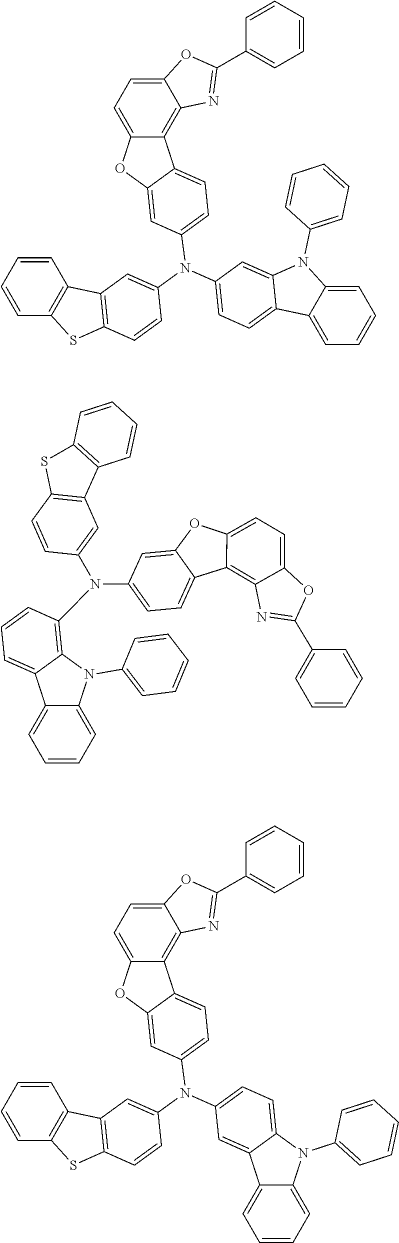 [SMILES: c1ccc(-c2nc3c(ccc4oc5cc(N(c6ccc7sc8ccccc8c7c6)c6ccc7c8ccccc8n(-c8ccccc8)c7c6)ccc5c43)o2)cc1 ; c1ccc(-c2nc3c(ccc4oc5cc(N(c6ccc7sc8ccccc8c7c6)c6cccc7c8ccccc8n(-c8ccccc8)c67)ccc5c43)o2)cc1 ; c1ccc(-c2nc3c(ccc4oc5cc(N(c6ccc7sc8ccccc8c7c6)c6ccc7c(c6)c6ccccc6n7-c6ccccc6)ccc5c43)o2)cc1]

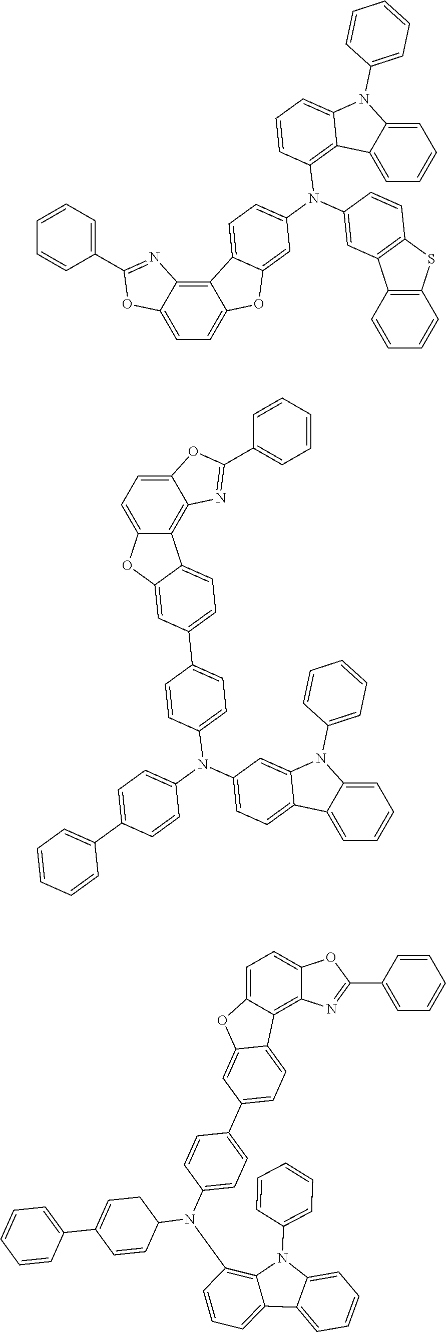 [SMILES: c1ccc(-c2nc3c(ccc4oc5cc(N(c6ccc7sc8ccccc8c7c6)c6cccc7c6c6ccccc6n7-c6ccccc6)ccc5c43)o2)cc1 ; c1ccc(-c2ccc(N(c3ccc(-c4ccc5c(c4)oc4ccc6oc(-c7ccccc7)nc6c45)cc3)c3ccc4c5ccccc5n(-c5ccccc5)c4c3)cc2)cc1 ; C1=CC(N(c2ccc(-c3ccc4c(c3)oc3ccc5oc(-c6ccccc6)nc5c34)cc2)c2cccc3c4ccccc4n(-c4ccccc4)c23)CC=C1c1ccccc1]

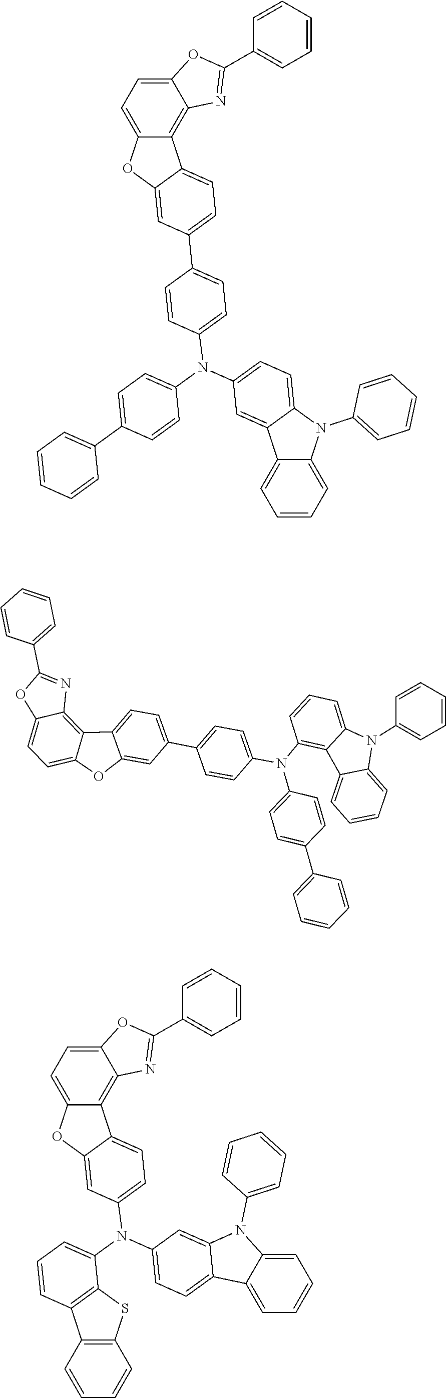 [SMILES: c1ccc(-c2ccc(N(c3ccc(-c4ccc5c(c4)oc4ccc6oc(-c7ccccc7)nc6c45)cc3)c3ccc4c(c3)c3ccccc3n4-c3ccccc3)cc2)cc1 ; c1ccc(-c2ccc(N(c3ccc(-c4ccc5c(c4)oc4ccc6oc(-c7ccccc7)nc6c45)cc3)c3cccc4c3c3ccccc3n4-c3ccccc3)cc2)cc1 ; c1ccc(-c2nc3c(ccc4oc5cc(N(c6ccc7c8ccccc8n(-c8ccccc8)c7c6)c6cccc7c6sc6ccccc67)ccc5c43)o2)cc1]

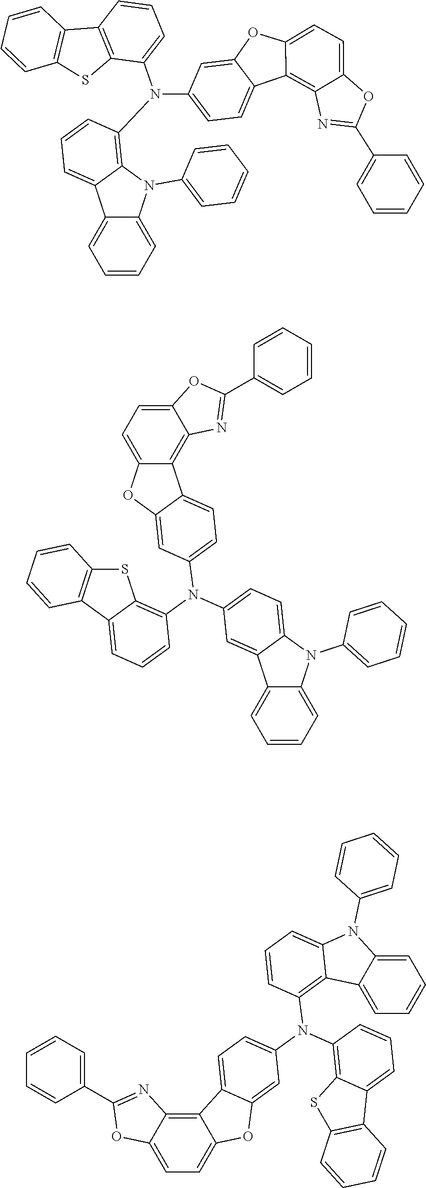 [SMILES: c1ccc(-c2nc3c(ccc4oc5cc(N(c6cccc7c6sc6ccccc67)c6cccc7c8ccccc8n(-c8ccccc8)c67)ccc5c43)o2)cc1 ; c1ccc(-c2nc3c(ccc4oc5cc(N(c6ccc7c(c6)c6ccccc6n7-c6ccccc6)c6cccc7c6sc6ccccc67)ccc5c43)o2)cc1 ; c1ccc(-c2nc3c(ccc4oc5cc(N(c6cccc7c6sc6ccccc67)c6cccc7c6c6ccccc6n7-c6ccccc6)ccc5c43)o2)cc1]

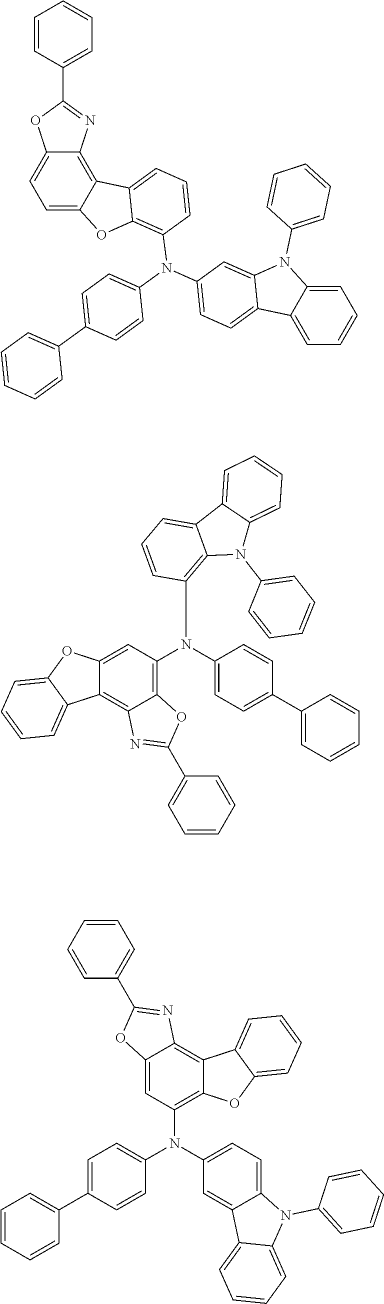 [SMILES: c1ccc(-c2ccc(N(c3ccc4c5ccccc5n(-c5ccccc5)c4c3)c3cccc4c3oc3ccc5oc(-c6ccccc6)nc5c34)cc2)cc1 ; c1ccc(-c2ccc(N(c3cc4oc5ccccc5c4c4nc(-c5ccccc5)oc34)c3cccc4c5ccccc5n(-c5ccccc5)c34)cc2)cc1 ; c1ccc(-c2ccc(N(c3ccc4c(c3)c3ccccc3n4-c3ccccc3)c3cc4oc(-c5ccccc5)nc4c4c3oc3ccccc34)cc2)cc1]

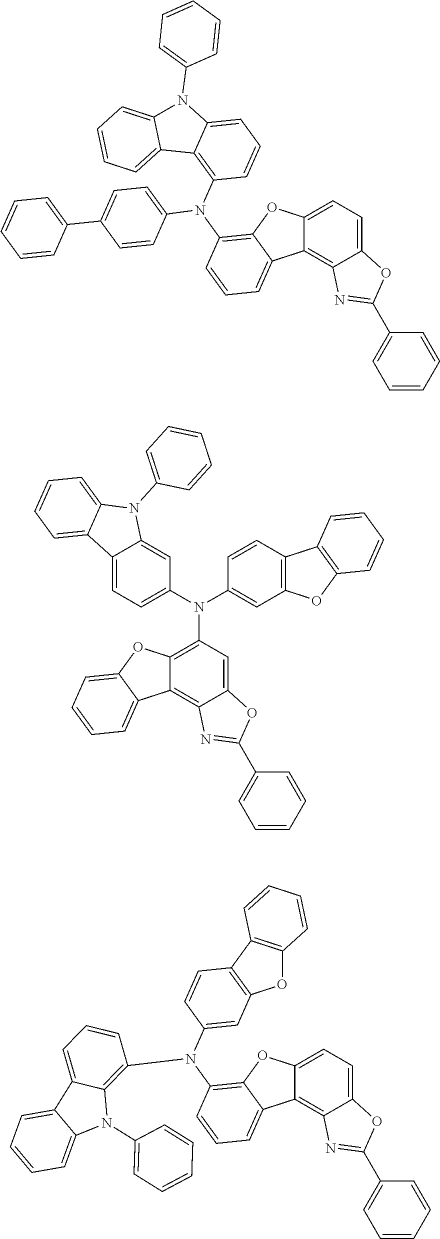 [SMILES: c1ccc(-c2ccc(N(c3cccc4c3oc3ccc5oc(-c6ccccc6)nc5c34)c3cccc4c3c3ccccc3n4-c3ccccc3)cc2)cc1 ; c1ccc(-c2nc3c(cc(N(c4ccc5c(c4)oc4ccccc45)c4ccc5c6ccccc6n(-c6ccccc6)c5c4)c4oc5ccccc5c43)o2)cc1 ; c1ccc(-c2nc3c(ccc4oc5c(N(c6ccc7c(c6)oc6ccccc67)c6cccc7c8ccccc8n(-c8ccccc8)c67)cccc5c43)o2)cc1]

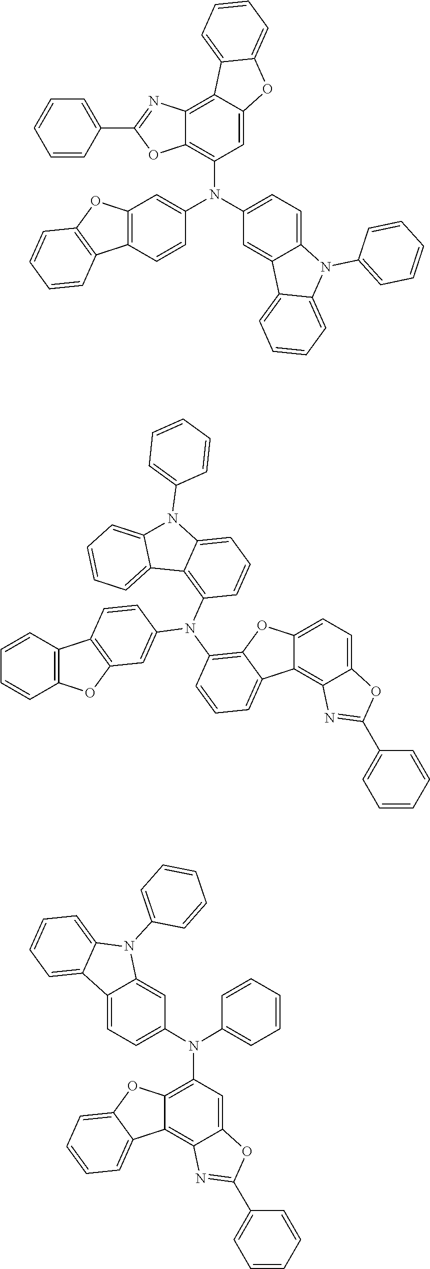 [SMILES: c1ccc(-c2nc3c(o2)c(N(c2ccc4c(c2)oc2ccccc24)c2ccc4c(c2)c2ccccc2n4-c2ccccc2)cc2oc4ccccc4c23)cc1 ; c1ccc(-c2nc3c(ccc4oc5c(N(c6ccc7c(c6)oc6ccccc67)c6cccc7c6c6ccccc6n7-c6ccccc6)cccc5c43)o2)cc1 ; c1ccc(-c2nc3c(cc(N(c4ccccc4)c4ccc5c6ccccc6n(-c6ccccc6)c5c4)c4oc5ccccc5c43)o2)cc1]

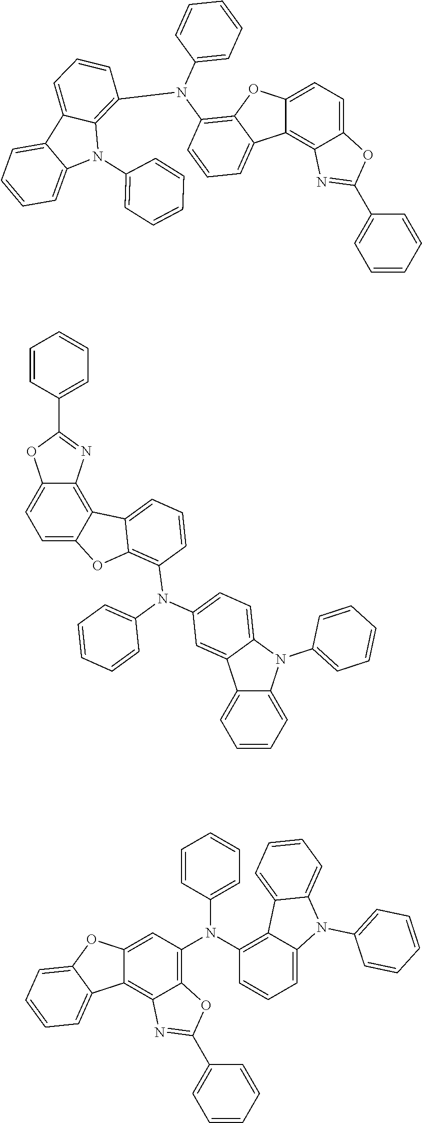 [SMILES: c1ccc(-c2nc3c(ccc4oc5c(N(c6ccccc6)c6cccc7c8ccccc8n(-c8ccccc8)c67)cccc5c43)o2)cc1 ; c1ccc(-c2nc3c(ccc4oc5c(N(c6ccccc6)c6ccc7c(c6)c6ccccc6n7-c6ccccc6)cccc5c43)o2)cc1 ; c1ccc(-c2nc3c(o2)c(N(c2ccccc2)c2cccc4c2c2ccccc2n4-c2ccccc2)cc2oc4ccccc4c23)cc1]

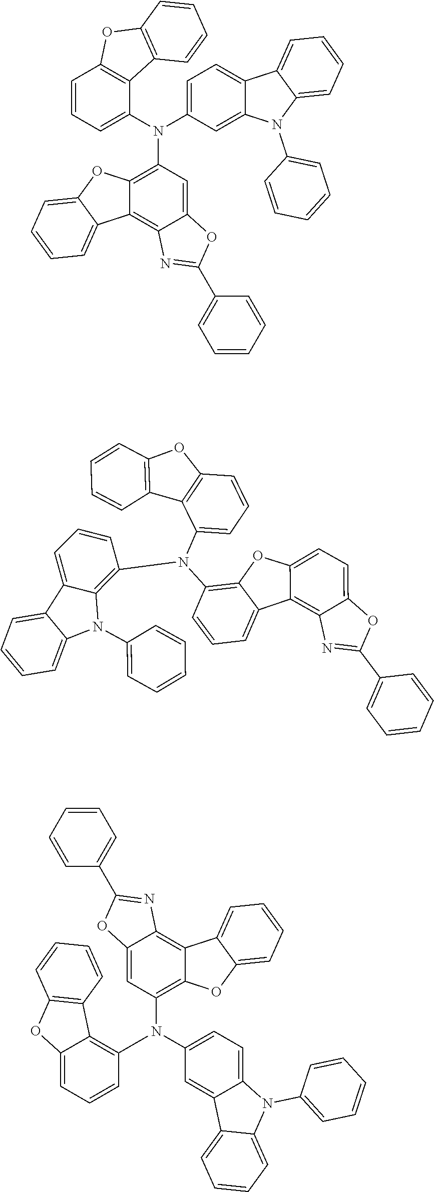 [SMILES: c1ccc(-c2nc3c(cc(N(c4ccc5c6ccccc6n(-c6ccccc6)c5c4)c4cccc5oc6ccccc6c45)c4oc5ccccc5c43)o2)cc1 ; c1ccc(-c2nc3c(ccc4oc5c(N(c6cccc7oc8ccccc8c67)c6cccc7c8ccccc8n(-c8ccccc8)c67)cccc5c43)o2)cc1 ; c1ccc(-c2nc3c(cc(N(c4ccc5c(c4)c4ccccc4n5-c4ccccc4)c4cccc5oc6ccccc6c45)c4oc5ccccc5c43)o2)cc1]

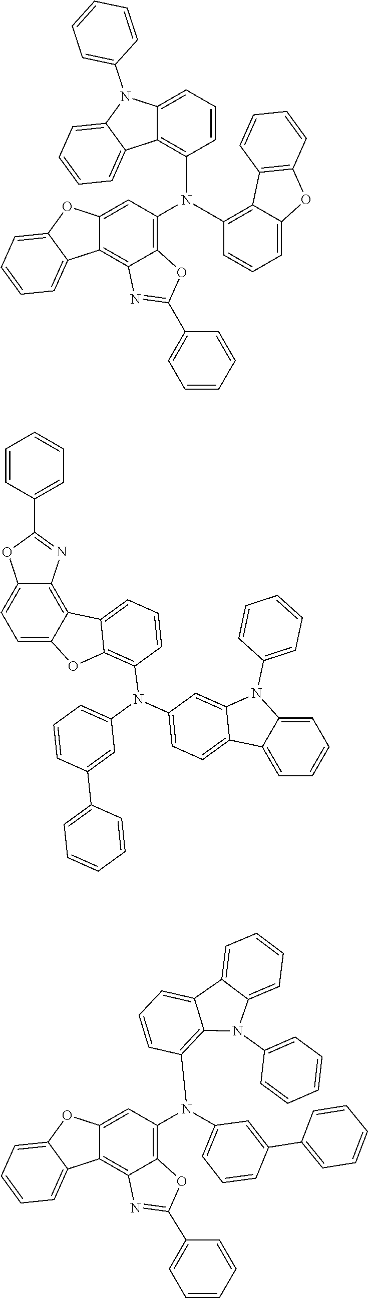 [SMILES: c1ccc(-c2nc3c(o2)c(N(c2cccc4oc5ccccc5c24)c2cccc4c2c2ccccc2n4-c2ccccc2)cc2oc4ccccc4c23)cc1 ; c1ccc(-c2cccc(N(c3ccc4c5ccccc5n(-c5ccccc5)c4c3)c3cccc4c3oc3ccc5oc(-c6ccccc6)nc5c34)c2)cc1 ; c1ccc(-c2cccc(N(c3cc4oc5ccccc5c4c4nc(-c5ccccc5)oc34)c3cccc4c5ccccc5n(-c5ccccc5)c34)c2)cc1]

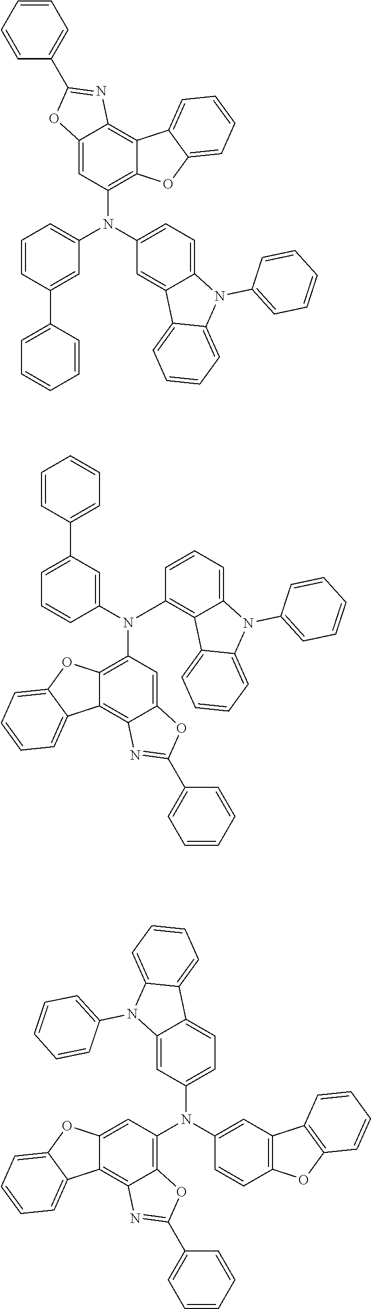 [SMILES: c1ccc(-c2cccc(N(c3ccc4c(c3)c3ccccc3n4-c3ccccc3)c3cc4oc(-c5ccccc5)nc4c4c3oc3ccccc34)c2)cc1 ; c1ccc(-c2cccc(N(c3cc4oc(-c5ccccc5)nc4c4c3oc3ccccc34)c3cccc4c3c3ccccc3n4-c3ccccc3)c2)cc1 ; c1ccc(-c2nc3c(o2)c(N(c2ccc4oc5ccccc5c4c2)c2ccc4c5ccccc5n(-c5ccccc5)c4c2)cc2oc4ccccc4c23)cc1]

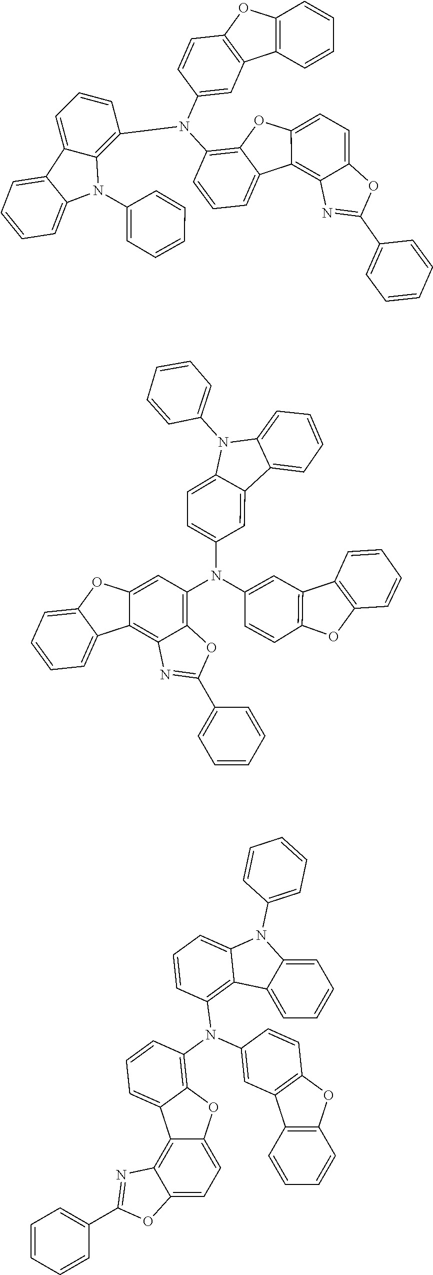 [SMILES: c1ccc(-c2nc3c(ccc4oc5c(N(c6ccc7oc8ccccc8c7c6)c6cccc7c8ccccc8n(-c8ccccc8)c67)cccc5c43)o2)cc1 ; c1ccc(-c2nc3c(o2)c(N(c2ccc4oc5ccccc5c4c2)c2ccc4c(c2)c2ccccc2n4-c2ccccc2)cc2oc4ccccc4c23)cc1 ; c1ccc(-c2nc3c(ccc4oc5c(N(c6ccc7oc8ccccc8c7c6)c6cccc7c6c6ccccc6n7-c6ccccc6)cccc5c43)o2)cc1]

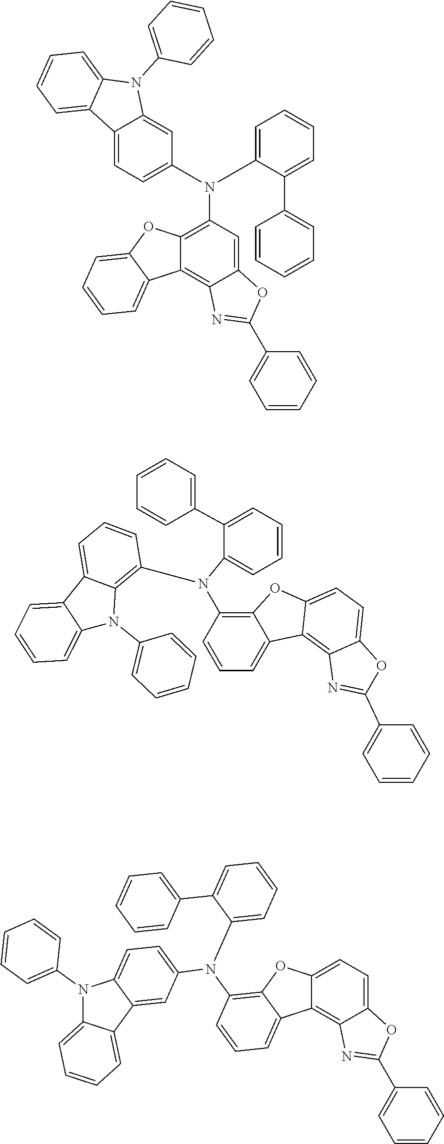 [SMILES: c1ccc(-c2nc3c(cc(N(c4ccc5c6ccccc6n(-c6ccccc6)c5c4)c4ccccc4-c4ccccc4)c4oc5ccccc5c43)o2)cc1 ; c1ccc(-c2nc3c(ccc4oc5c(N(c6ccccc6-c6ccccc6)c6cccc7c8ccccc8n(-c8ccccc8)c67)cccc5c43)o2)cc1 ; c1ccc(-c2nc3c(ccc4oc5c(N(c6ccc7c(c6)c6ccccc6n7-c6ccccc6)c6ccccc6-c6ccccc6)cccc5c43)o2)cc1]

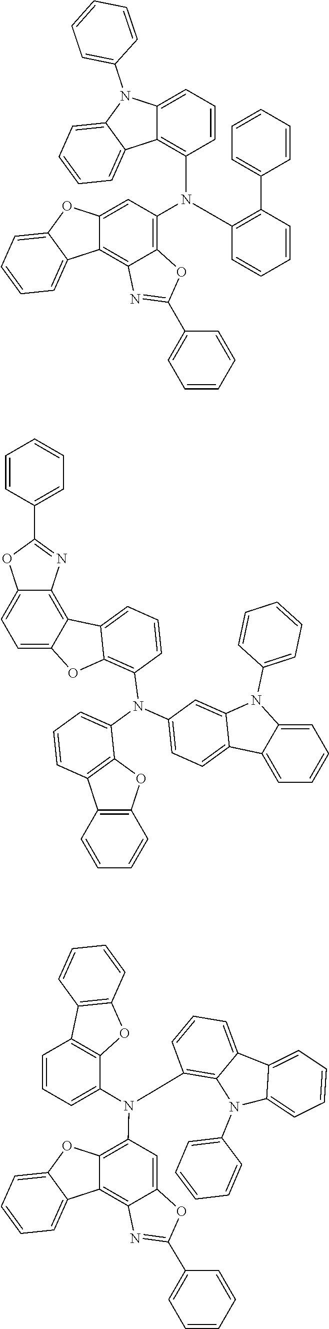 [SMILES: c1ccc(-c2nc3c(o2)c(N(c2ccccc2-c2ccccc2)c2cccc4c2c2ccccc2n4-c2ccccc2)cc2oc4ccccc4c23)cc1 ; c1ccc(-c2nc3c(ccc4oc5c(N(c6ccc7c8ccccc8n(-c8ccccc8)c7c6)c6cccc7c6oc6ccccc67)cccc5c43)o2)cc1 ; c1ccc(-c2nc3c(cc(N(c4cccc5c4oc4ccccc45)c4cccc5c6ccccc6n(-c6ccccc6)c45)c4oc5ccccc5c43)o2)cc1]

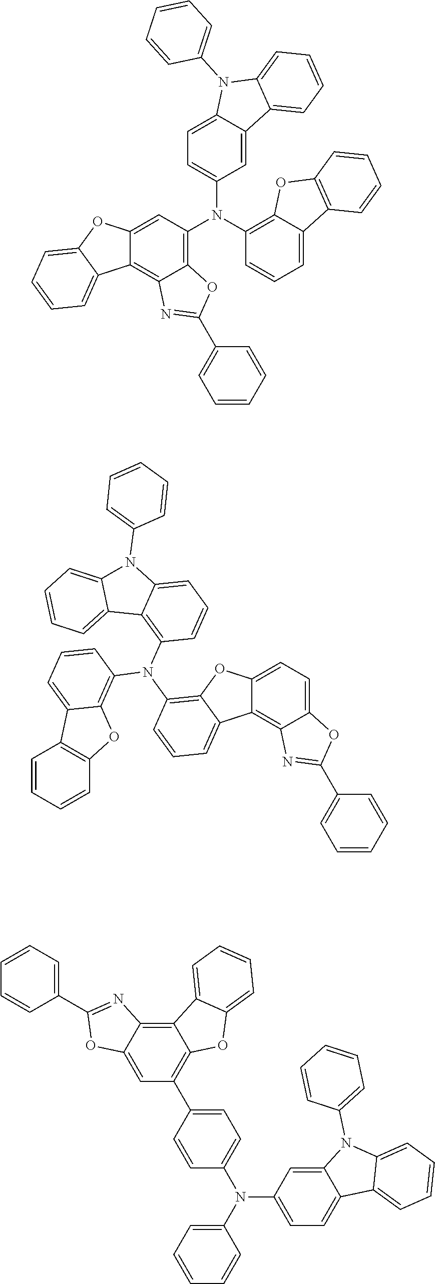 [SMILES: c1ccc(-c2nc3c(o2)c(N(c2ccc4c(c2)c2ccccc2n4-c2ccccc2)c2cccc4c2oc2ccccc24)cc2oc4ccccc4c23)cc1 ; c1ccc(-c2nc3c(ccc4oc5c(N(c6cccc7c6oc6ccccc67)c6cccc7c6c6ccccc6n7-c6ccccc6)cccc5c43)o2)cc1 ; c1ccc(-c2nc3c(cc(-c4ccc(N(c5ccccc5)c5ccc6c7ccccc7n(-c7ccccc7)c6c5)cc4)c4oc5ccccc5c43)o2)cc1]

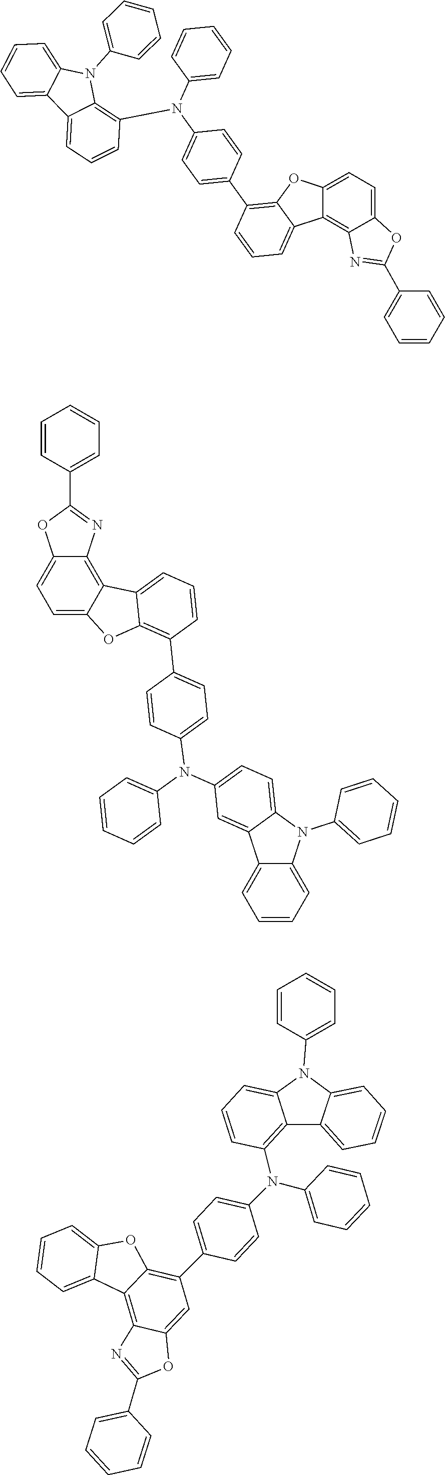 [SMILES: c1ccc(-c2nc3c(ccc4oc5c(-c6ccc(N(c7ccccc7)c7cccc8c9ccccc9n(-c9ccccc9)c78)cc6)cccc5c43)o2)cc1 ; c1ccc(-c2nc3c(ccc4oc5c(-c6ccc(N(c7ccccc7)c7ccc8c(c7)c7ccccc7n8-c7ccccc7)cc6)cccc5c43)o2)cc1 ; c1ccc(-c2nc3c(cc(-c4ccc(N(c5ccccc5)c5cccc6c5c5ccccc5n6-c5ccccc5)cc4)c4oc5ccccc5c43)o2)cc1]

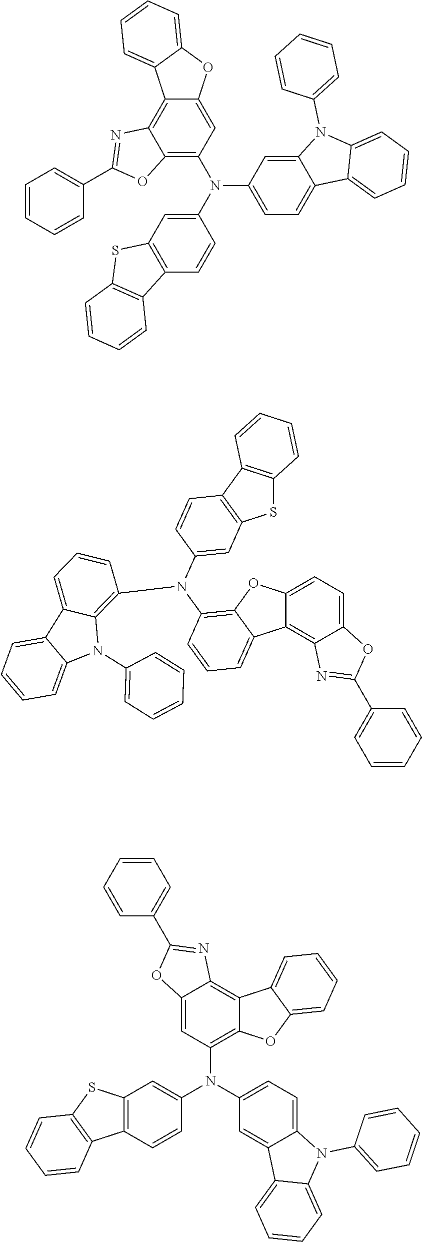 [SMILES: c1ccc(-c2nc3c(o2)c(N(c2ccc4c(c2)sc2ccccc24)c2ccc4c5ccccc5n(-c5ccccc5)c4c2)cc2oc4ccccc4c23)cc1 ; c1ccc(-c2nc3c(ccc4oc5c(N(c6ccc7c(c6)sc6ccccc67)c6cccc7c8ccccc8n(-c8ccccc8)c67)cccc5c43)o2)cc1 ; c1ccc(-c2nc3c(cc(N(c4ccc5c(c4)sc4ccccc45)c4ccc5c(c4)c4ccccc4n5-c4ccccc4)c4oc5ccccc5c43)o2)cc1]

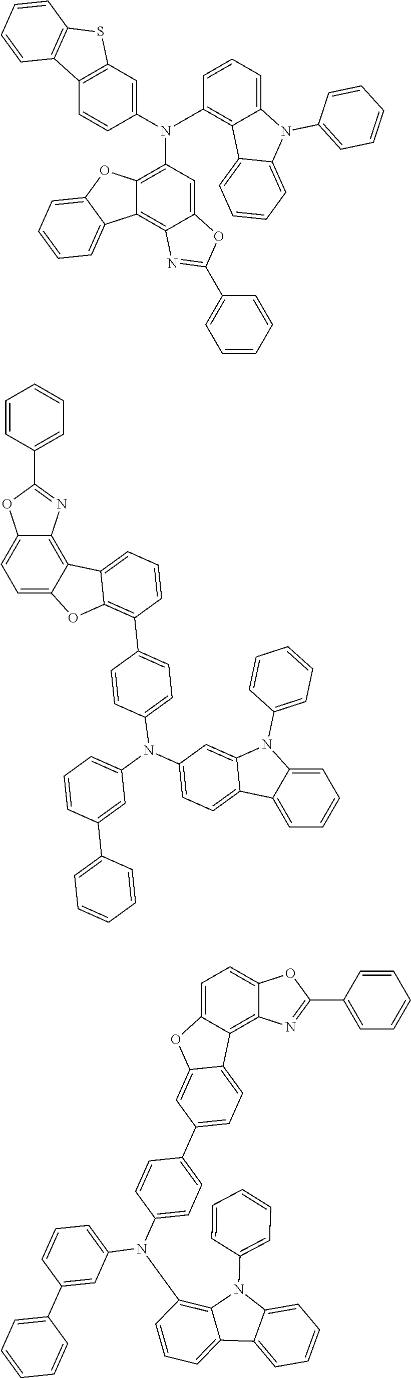 [SMILES: c1ccc(-c2nc3c(cc(N(c4ccc5c(c4)sc4ccccc45)c4cccc5c4c4ccccc4n5-c4ccccc4)c4oc5ccccc5c43)o2)cc1 ; c1ccc(-c2cccc(N(c3ccc(-c4cccc5c4oc4ccc6oc(-c7ccccc7)nc6c45)cc3)c3ccc4c5ccccc5n(-c5ccccc5)c4c3)c2)cc1 ; c1ccc(-c2cccc(N(c3ccc(-c4ccc5c(c4)oc4ccc6oc(-c7ccccc7)nc6c45)cc3)c3cccc4c5ccccc5n(-c5ccccc5)c34)c2)cc1]

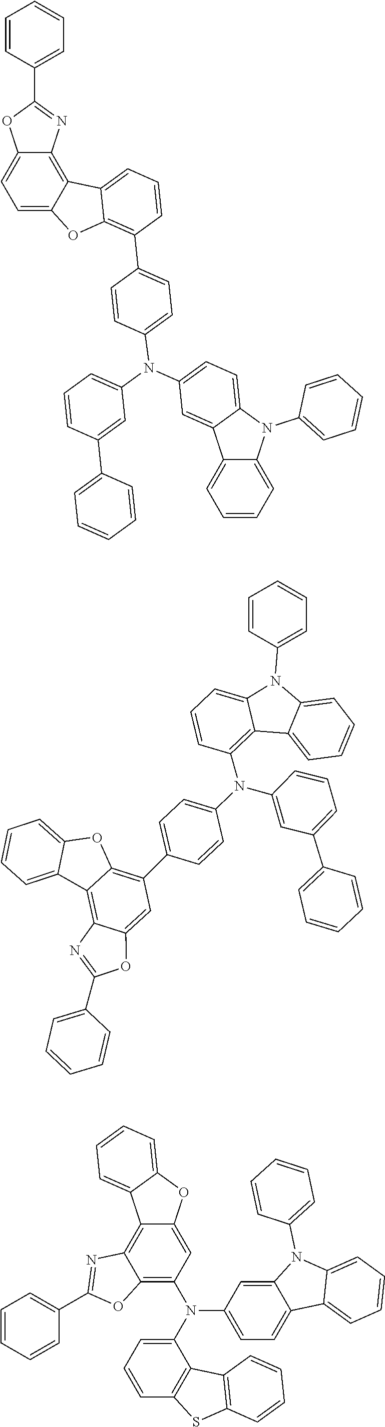 [SMILES: c1ccc(-c2cccc(N(c3ccc(-c4cccc5c4oc4ccc6oc(-c7ccccc7)nc6c45)cc3)c3ccc4c(c3)c3ccccc3n4-c3ccccc3)c2)cc1 ; c1ccc(-c2cccc(N(c3ccc(-c4cc5oc(-c6ccccc6)nc5c5c4oc4ccccc45)cc3)c3cccc4c3c3ccccc3n4-c3ccccc3)c2)cc1 ; c1ccc(-c2nc3c(o2)c(N(c2ccc4c5ccccc5n(-c5ccccc5)c4c2)c2cccc4sc5ccccc5c24)cc2oc4ccccc4c23)cc1]

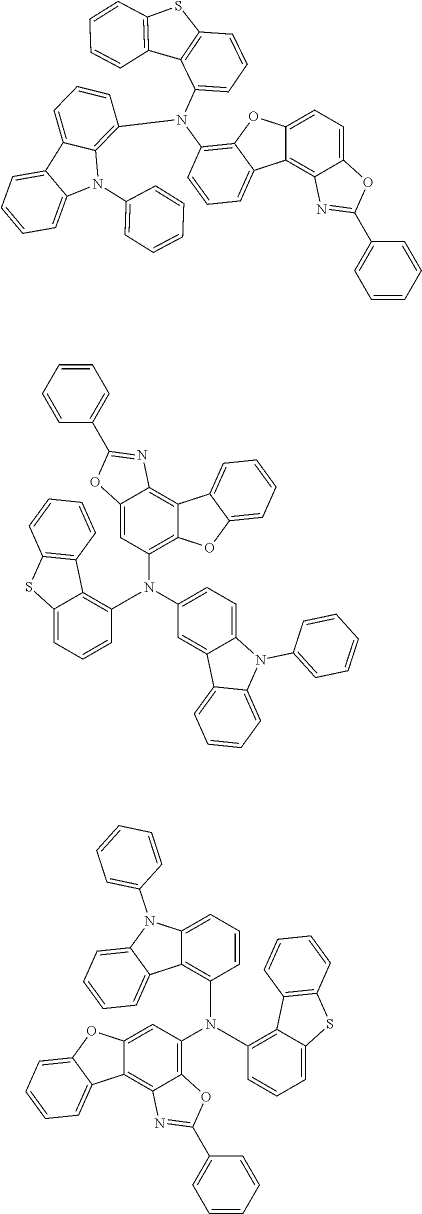 [SMILES: c1ccc(-c2nc3c(ccc4oc5c(N(c6cccc7sc8ccccc8c67)c6cccc7c8ccccc8n(-c8ccccc8)c67)cccc5c43)o2)cc1 ; c1ccc(-c2nc3c(cc(N(c4ccc5c(c4)c4ccccc4n5-c4ccccc4)c4cccc5sc6ccccc6c45)c4oc5ccccc5c43)o2)cc1 ; c1ccc(-c2nc3c(o2)c(N(c2cccc4sc5ccccc5c24)c2cccc4c2c2ccccc2n4-c2ccccc2)cc2oc4ccccc4c23)cc1]

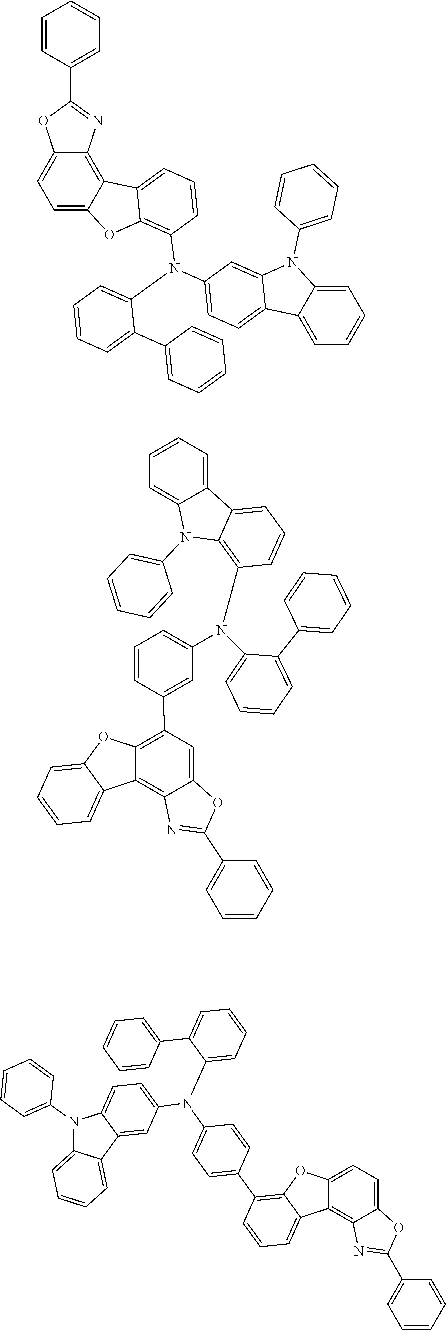 [SMILES: c1ccc(-c2nc3c(ccc4oc5c(N(c6ccc7c8ccccc8n(-c8ccccc8)c7c6)c6ccccc6-c6ccccc6)cccc5c43)o2)cc1 ; c1ccc(-c2nc3c(cc(-c4cccc(N(c5ccccc5-c5ccccc5)c5cccc6c7ccccc7n(-c7ccccc7)c56)c4)c4oc5ccccc5c43)o2)cc1 ; c1ccc(-c2nc3c(ccc4oc5c(-c6ccc(N(c7ccc8c(c7)c7ccccc7n8-c7ccccc7)c7ccccc7-c7ccccc7)cc6)cccc5c43)o2)cc1]

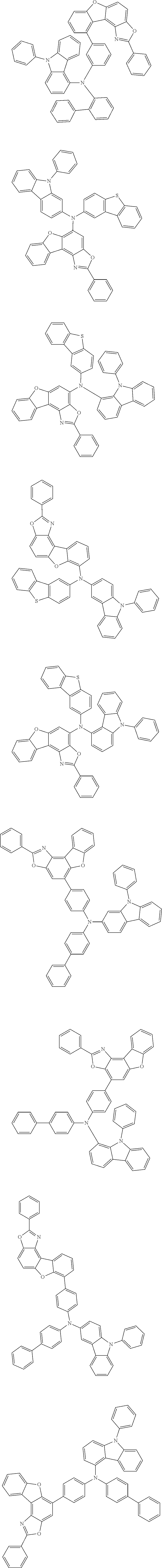

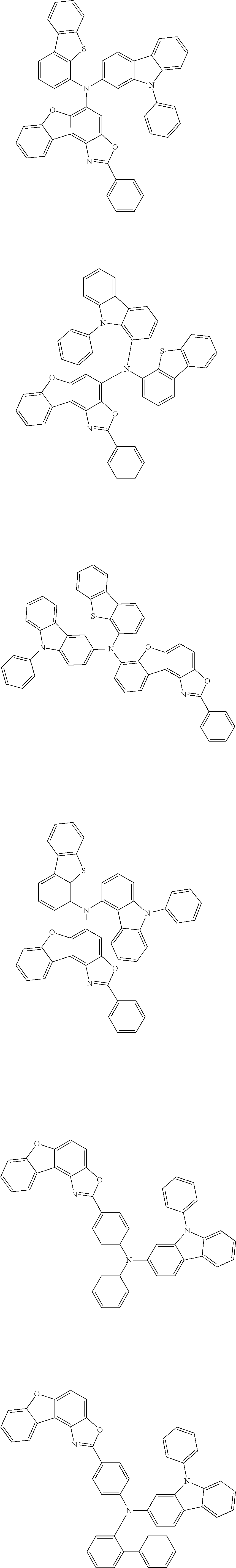

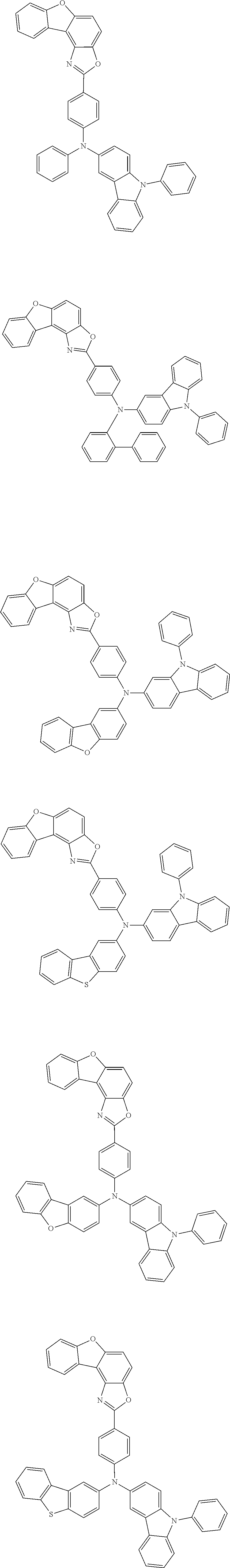

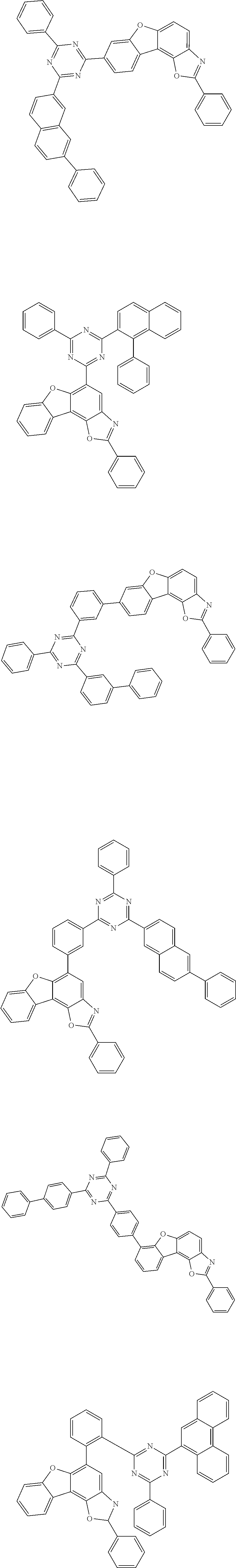

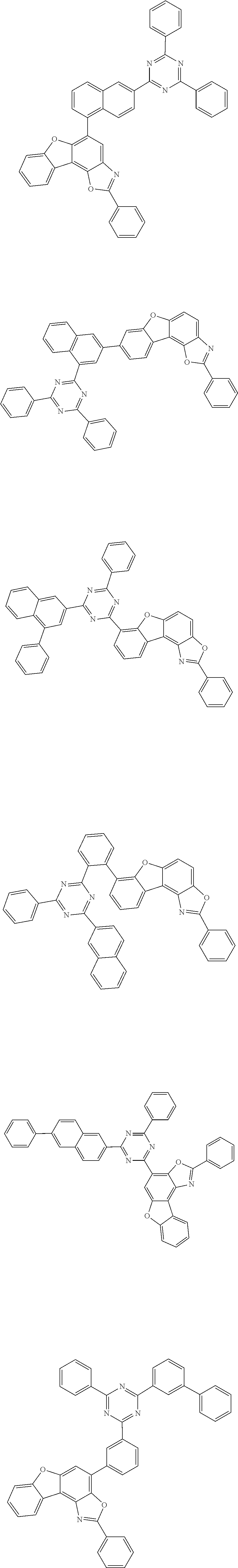

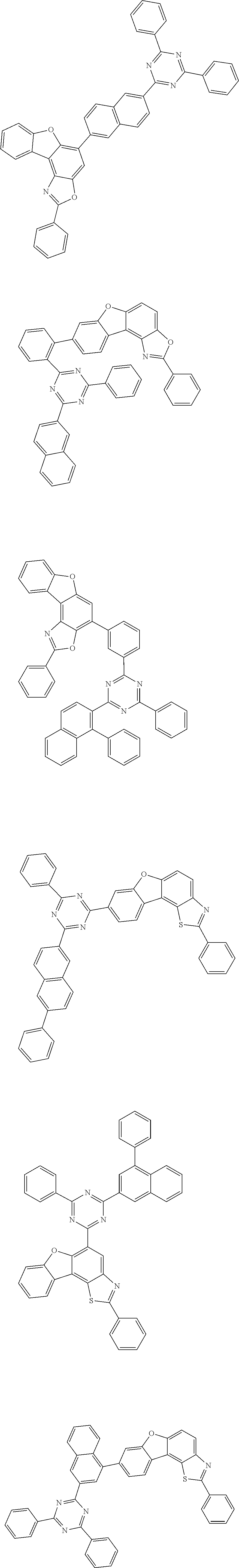

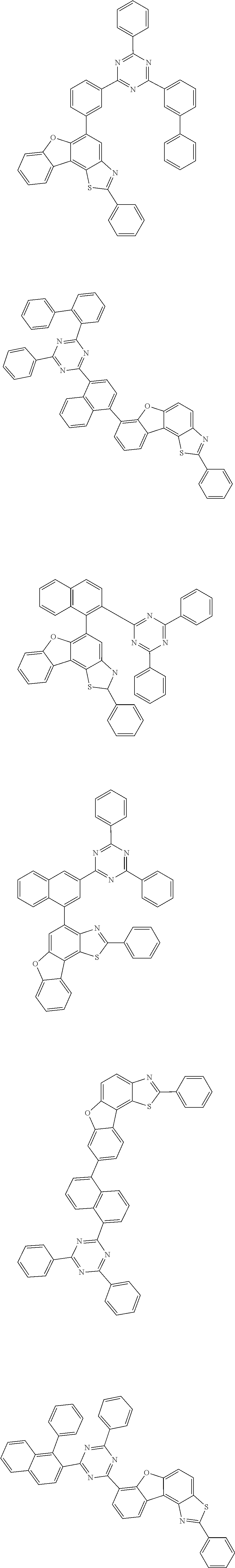

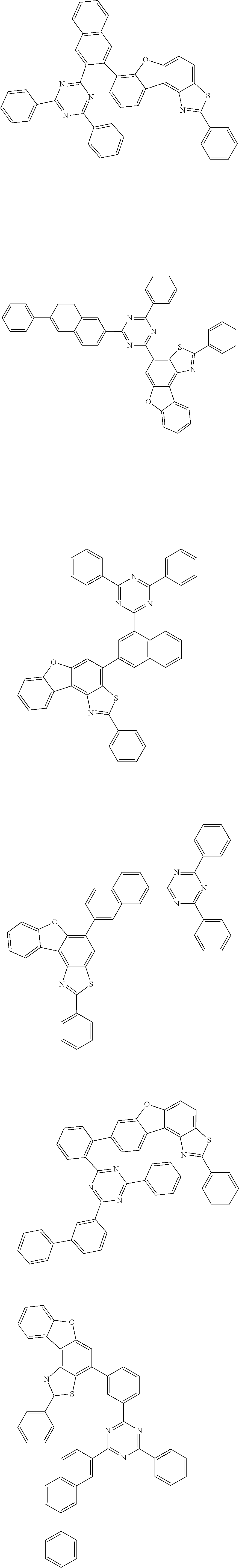

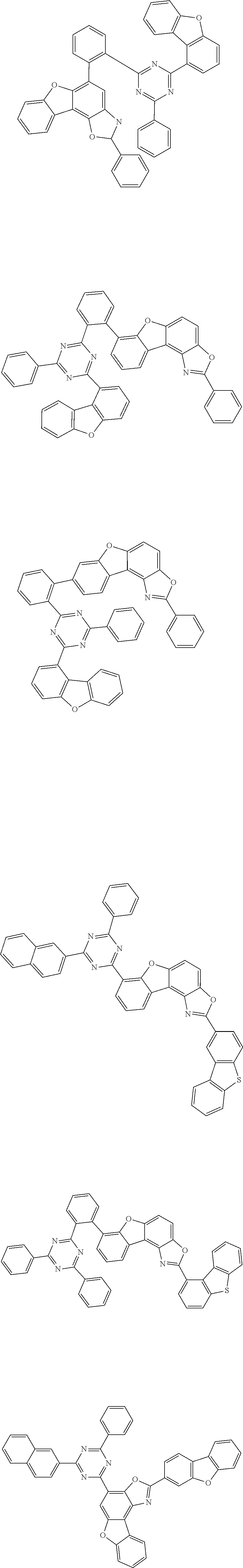

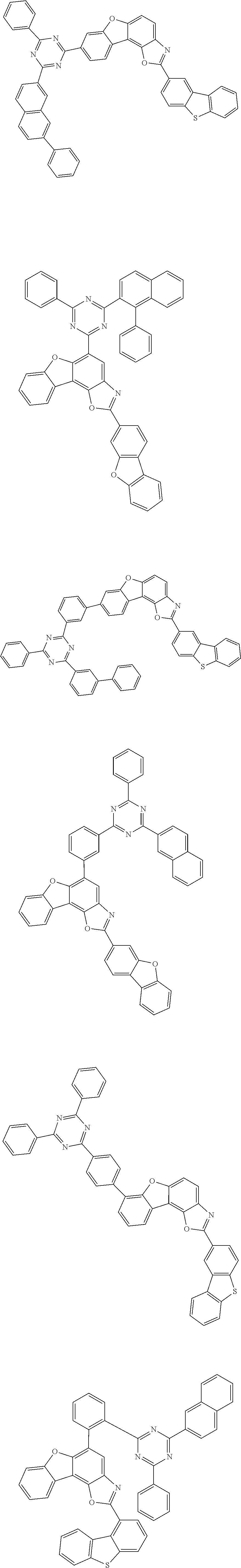

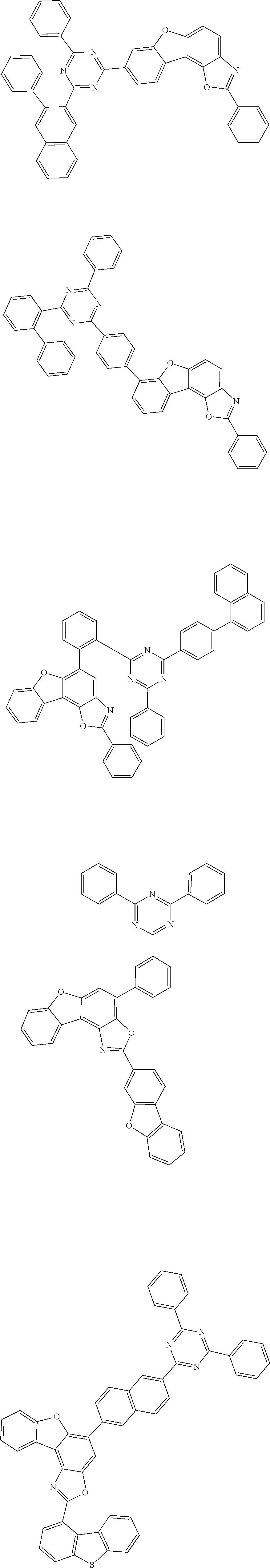 [SMILES: c1ccc(-c2nc(-c3ccc4c(c3)oc3ccc5nc(-c6ccccc6)oc5c34)nc(-c3cc4ccccc4cc3-c3ccccc3)n2)cc1 ; c1ccc(-c2nc(-c3ccc(-c4cccc5c4oc4ccc6nc(-c7ccccc7)oc6c45)cc3)nc(-c3ccccc3-c3ccccc3)n2)cc1 ; c1ccc(-c2nc(-c3ccc(-c4cccc5ccccc45)cc3)nc(-c3ccccc3-c3cc4nc(-c5ccccc5)oc4c4c3oc3ccccc34)n2)cc1 ; c1ccc(-c2nc(-c3ccccc3)nc(-c3cccc(-c4cc5oc6ccccc6c5c5nc(-c6ccc7c(c6)oc6ccccc67)oc45)c3)n2)cc1 ; c1ccc(-c2nc(-c3ccccc3)nc(-c3ccc4cc(-c5cc6oc(-c7cccc8sc9ccccc9c78)nc6c6c5oc5ccccc56)ccc4c3)n2)cc1]

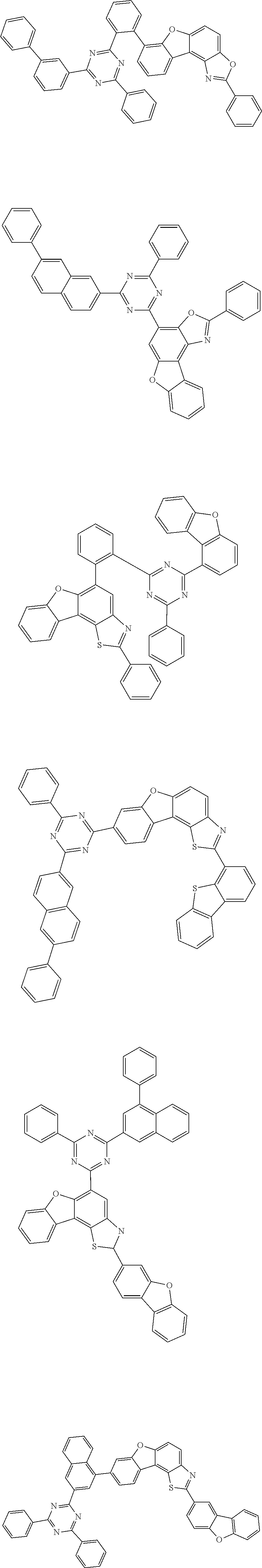

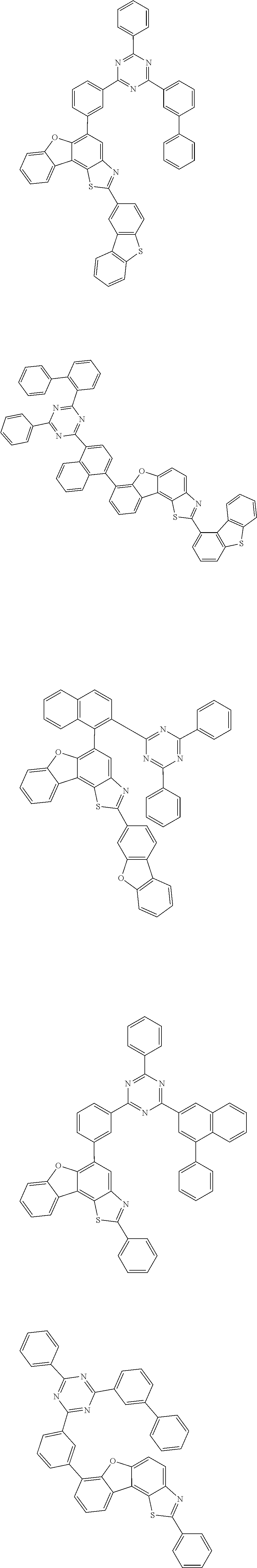 [SMILES: c1ccc(-c2cccc(-c3nc(-c4ccccc4)nc(-c4cccc(-c5cc6nc(-c7ccc8sc9ccccc9c8c7)sc6c6c5oc5ccccc56)c4)n3)c2)cc1 ; c1ccc(-c2nc(-c3ccccc3-c3ccccc3)nc(-c3ccc(-c4cccc5c4oc4ccc6nc(-c7cccc8sc9ccccc9c78)sc6c45)c4ccccc34)n2)cc1 ; c1ccc(-c2nc(-c3ccccc3)nc(-c3ccc4ccccc4c3-c3cc4nc(-c5ccc6c(c5)oc5ccccc56)sc4c4c3oc3ccccc34)n2)cc1 ; c1ccc(-c2nc(-c3cccc(-c4cc5nc(-c6ccccc6)sc5c5c4oc4ccccc45)c3)nc(-c3cc(-c4ccccc4)c4ccccc4c3)n2)cc1 ; c1ccc(-c2cccc(-c3nc(-c4ccccc4)nc(-c4cccc(-c5cccc6c5oc5ccc7nc(-c8ccccc8)sc7c56)c4)n3)c2)cc1]

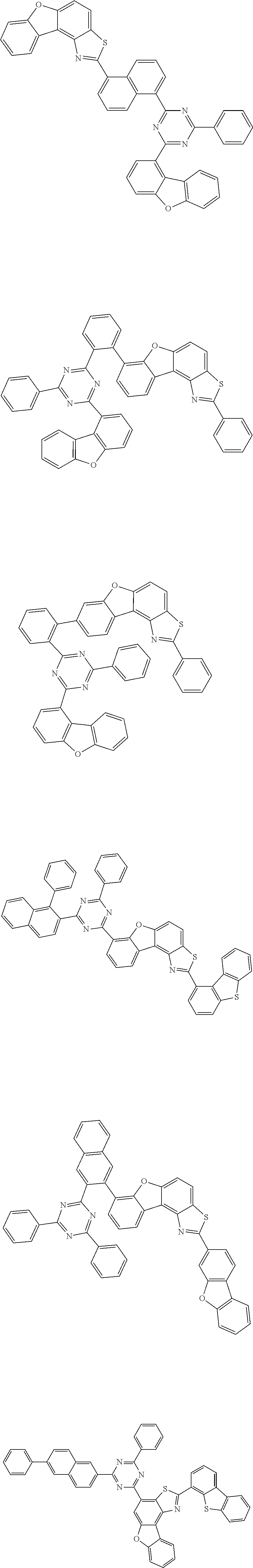

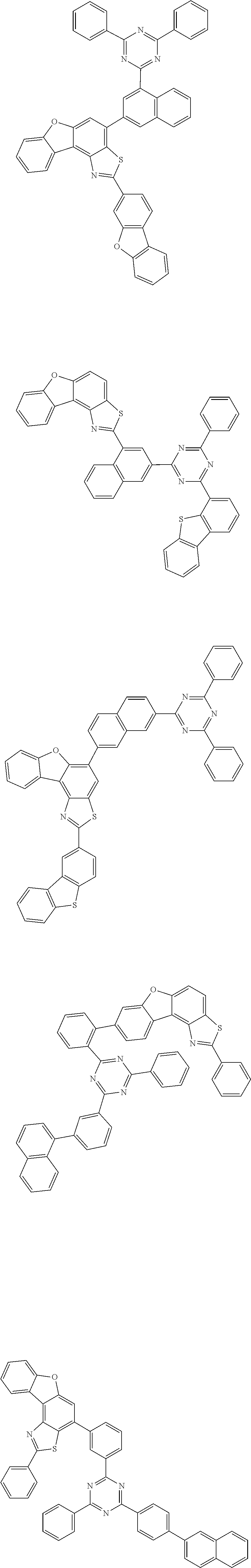 [SMILES: c1ccc(-c2nc(-c3ccccc3)nc(-c3cc(-c4cc5oc6ccccc6c5c5nc(-c6ccc7c(c6)oc6ccccc67)sc45)cc4ccccc34)n2)cc1 ; c1ccc(-c2nc(-c3cc(-c4nc5c(ccc6oc7ccccc7c65)s4)c4ccccc4c3)nc(-c3cccc4c3sc3ccccc34)n2)cc1 ; c1ccc(-c2nc(-c3ccccc3)nc(-c3ccc4ccc(-c5cc6sc(-c7ccc8sc9ccccc9c8c7)nc6c6c5oc5ccccc56)cc4c3)n2)cc1 ; c1ccc(-c2nc(-c3cccc(-c4cccc5ccccc45)c3)nc(-c3ccccc3-c3ccc4c(c3)oc3ccc5sc(-c6ccccc6)nc5c34)n2)cc1 ; c1ccc(-c2nc(-c3ccc(-c4ccc5ccccc5c4)cc3)nc(-c3cccc(-c4cc5oc6ccccc6c5c5nc(-c6ccccc6)sc45)c3)n2)cc1]

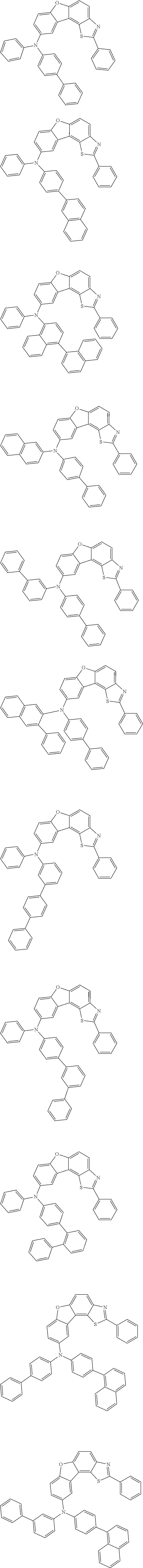

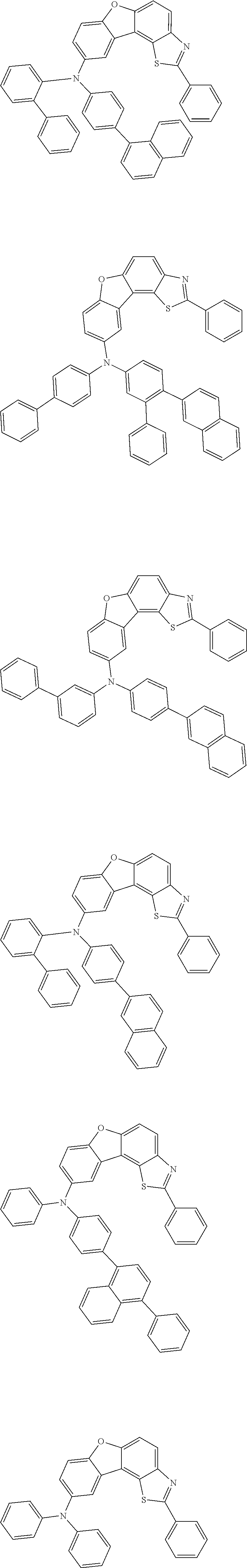 [SMILES: c1ccc(-c2nc3ccc4oc5ccc(N(c6ccc(-c7cccc8ccccc78)cc6)c6ccccc6-c6ccccc6)cc5c4c3s2)cc1 ; c1ccc(-c2ccc(N(c3ccc(-c4ccc5ccccc5c4)c(-c4ccccc4)c3)c3ccc4oc5ccc6nc(-c7ccccc7)sc6c5c4c3)cc2)cc1 ; c1ccc(-c2cccc(N(c3ccc(-c4ccc5ccccc5c4)cc3)c3ccc4oc5ccc6nc(-c7ccccc7)sc6c5c4c3)c2)cc1 ; c1ccc(-c2nc3ccc4oc5ccc(N(c6ccc(-c7ccc8ccccc8c7)cc6)c6ccccc6-c6ccccc6)cc5c4c3s2)cc1 ; c1ccc(-c2nc3ccc4oc5ccc(N(c6ccccc6)c6ccc(-c7ccc(-c8ccccc8)c8ccccc78)cc6)cc5c4c3s2)cc1 ; c1ccc(-c2nc3ccc4oc5ccc(N(c6ccccc6)c6ccccc6)cc5c4c3s2)cc1]

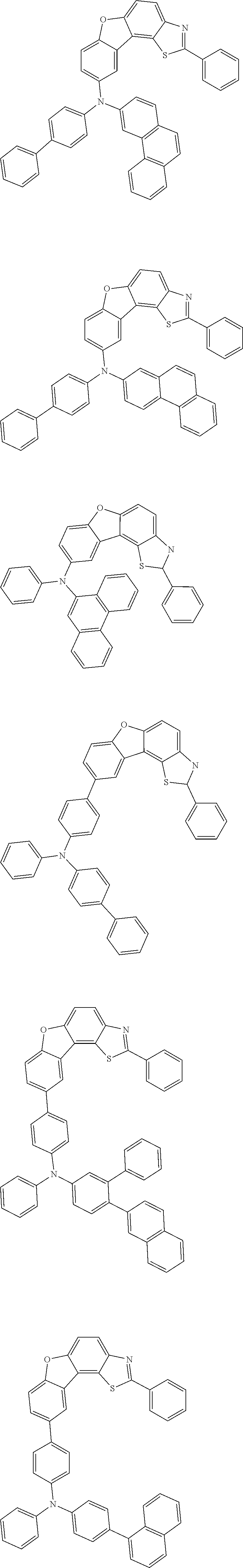

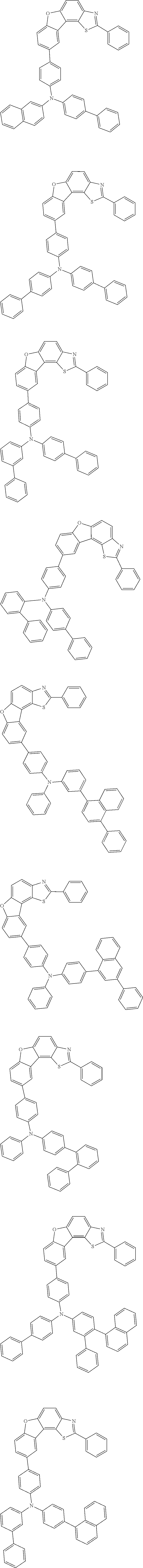

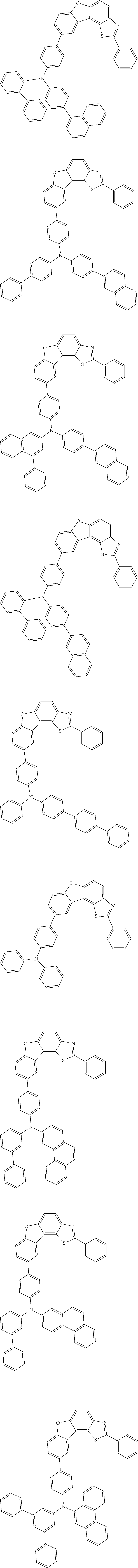

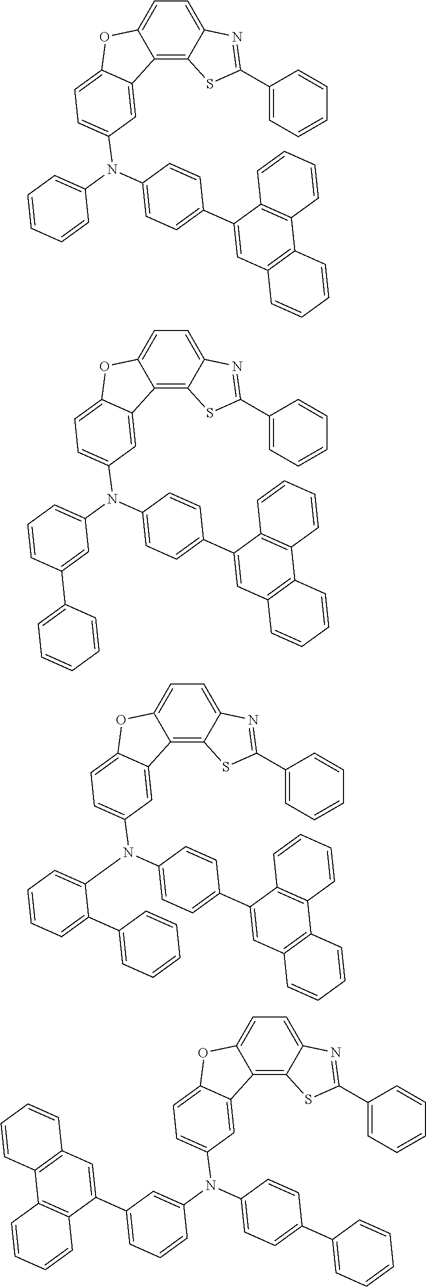

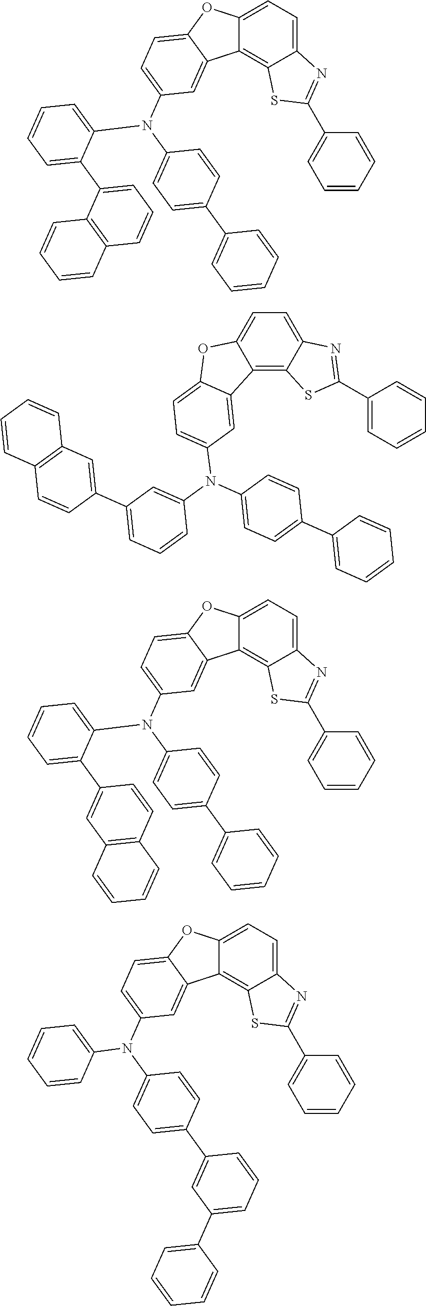

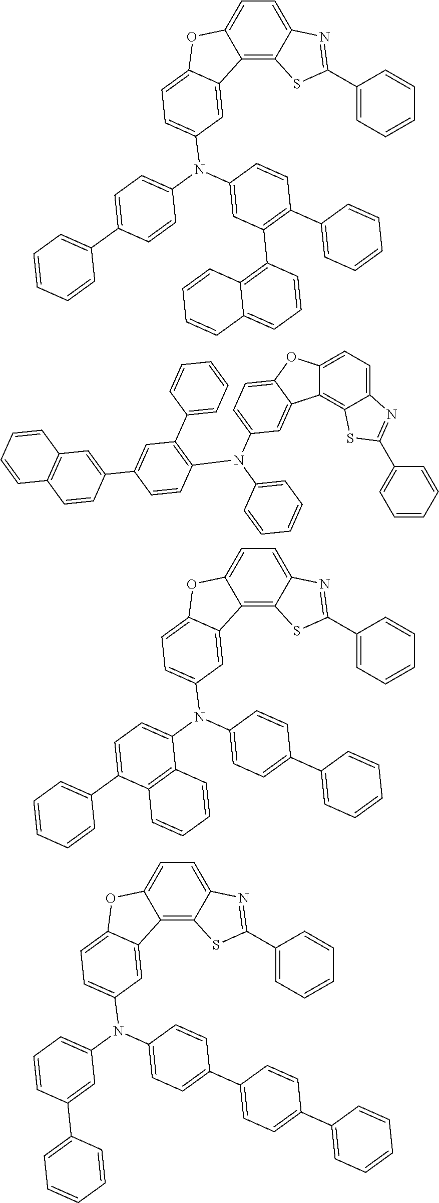

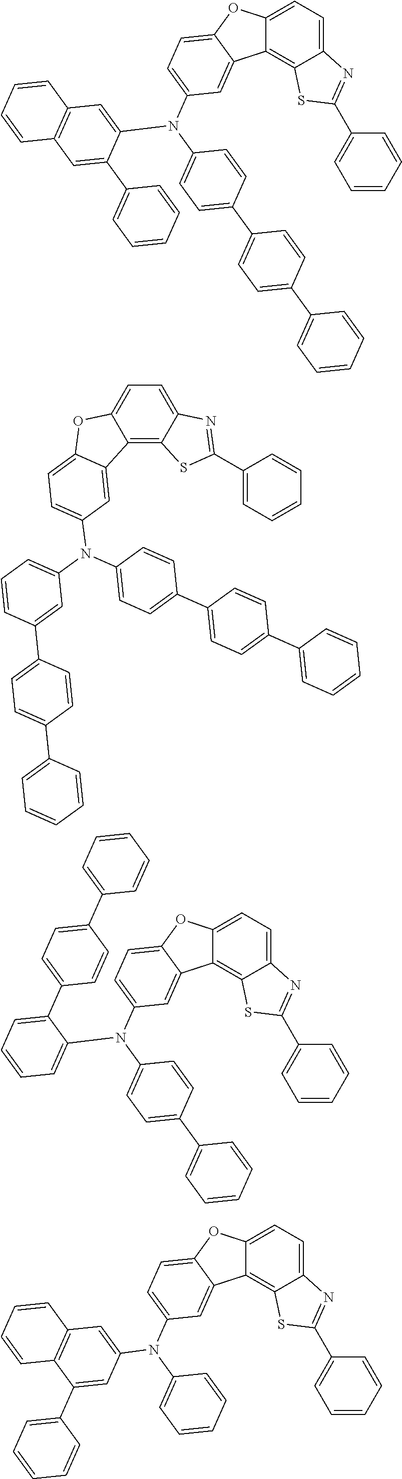

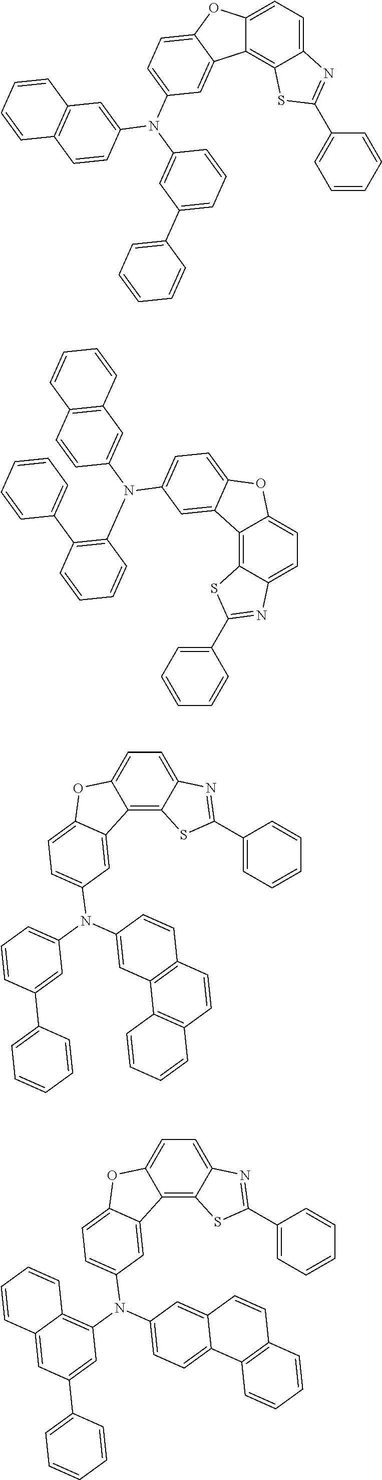 [SMILES: c1ccc(-c2cccc(N(c3ccc4ccccc4c3)c3ccc4oc5ccc6nc(-c7ccccc7)sc6c5c4c3)c2)cc1 ; c1ccc(-c2nc3ccc4oc5ccc(N(c6ccc7ccccc7c6)c6ccccc6-c6ccccc6)cc5c4c3s2)cc1 ; c1ccc(-c2cccc(N(c3ccc4ccc5ccccc5c4c3)c3ccc4oc5ccc6nc(-c7ccccc7)sc6c5c4c3)c2)cc1 ; c1ccc(-c2cc(N(c3ccc4c(ccc5ccccc54)c3)c3ccc4oc5ccc6nc(-c7ccccc7)sc6c5c4c3)c3ccccc3c2)cc1]

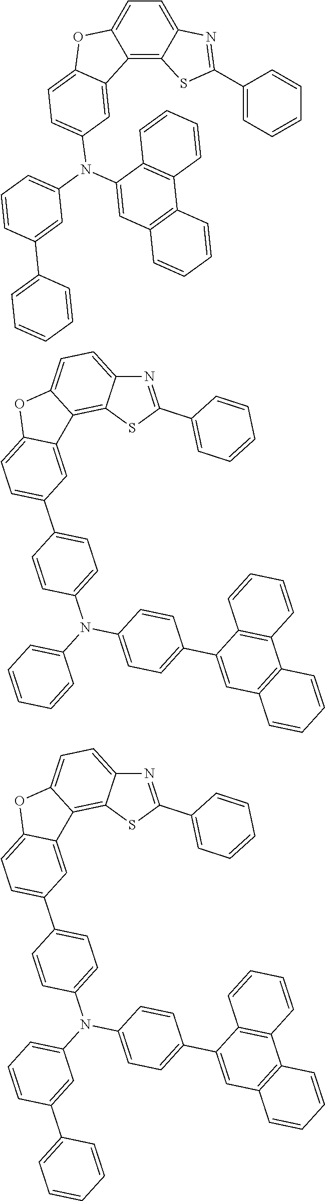

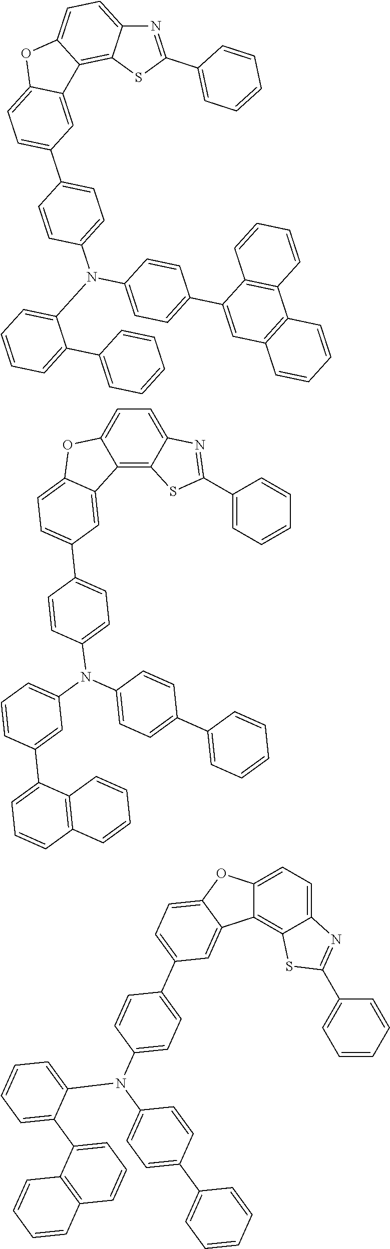

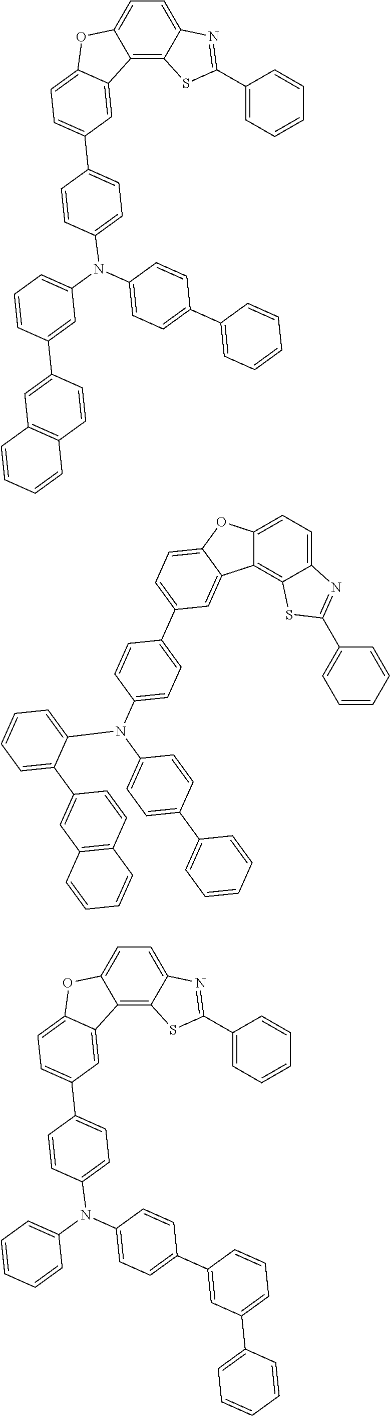

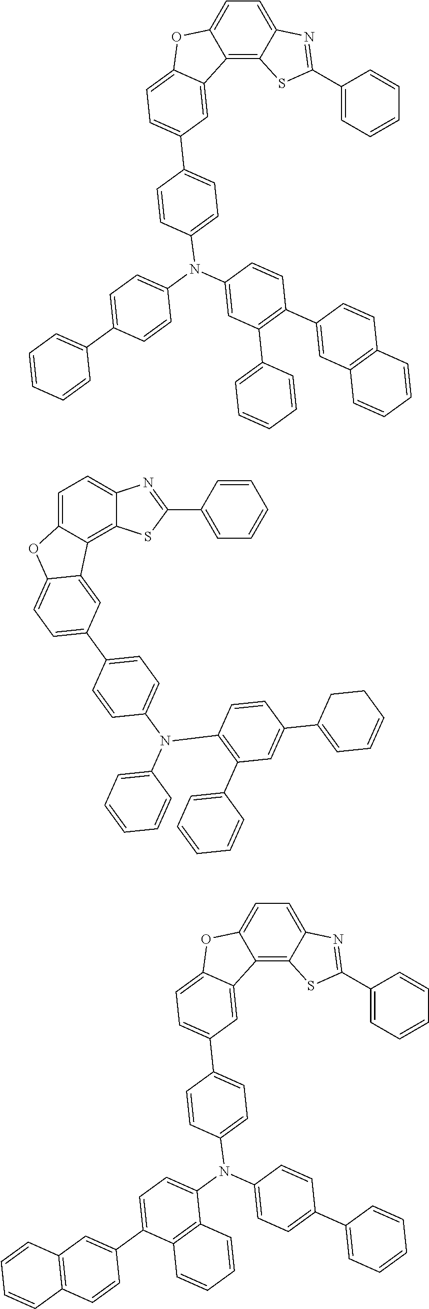 [SMILES: c1ccc(-c2ccc(N(c3ccc(-c4ccc5oc6ccc7nc(-c8ccccc8)sc7c6c5c4)cc3)c3ccc(-c4ccc5ccccc5c4)c(-c4ccccc4)c3)cc2)cc1 ; C1=CCCC(c2ccc(N(c3ccccc3)c3ccc(-c4ccc5oc6ccc7nc(-c8ccccc8)sc7c6c5c4)cc3)c(-c3ccccc3)c2)=C1 ; c1ccc(-c2ccc(N(c3ccc(-c4ccc5oc6ccc7nc(-c8ccccc8)sc7c6c5c4)cc3)c3ccc(-c4ccc5ccccc5c4)c4ccccc34)cc2)cc1]

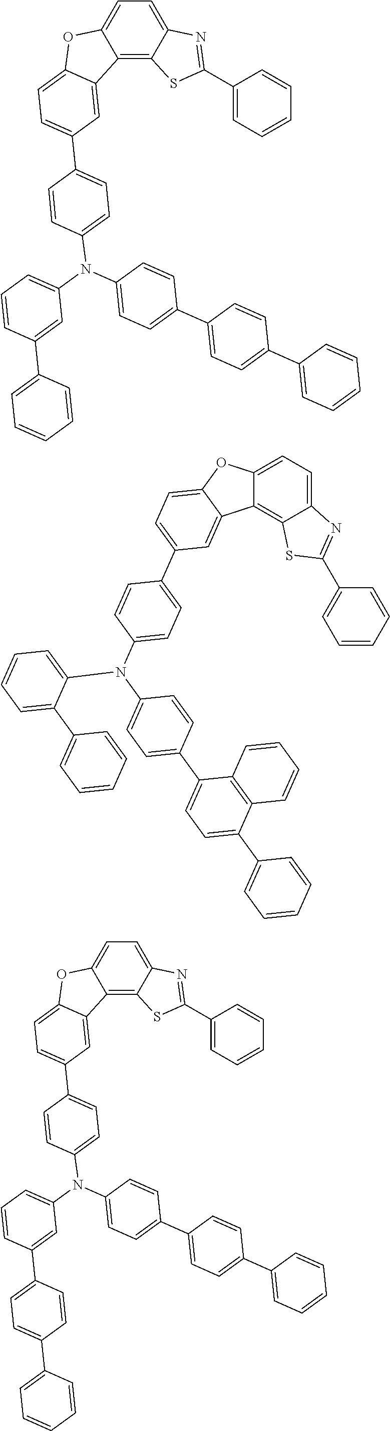 [SMILES: c1ccc(-c2ccc(-c3ccc(N(c4ccc(-c5ccc6oc7ccc8nc(-c9ccccc9)sc8c7c6c5)cc4)c4cccc(-c5ccccc5)c4)cc3)cc2)cc1 ; c1ccc(-c2nc3ccc4oc5ccc(-c6ccc(N(c7ccc(-c8ccc(-c9ccccc9)c9ccccc89)cc7)c7ccccc7-c7ccccc7)cc6)cc5c4c3s2)cc1 ; c1ccc(-c2ccc(-c3ccc(N(c4ccc(-c5ccc6oc7ccc8nc(-c9ccccc9)sc8c7c6c5)cc4)c4cccc(-c5ccc(-c6ccccc6)cc5)c4)cc3)cc2)cc1]

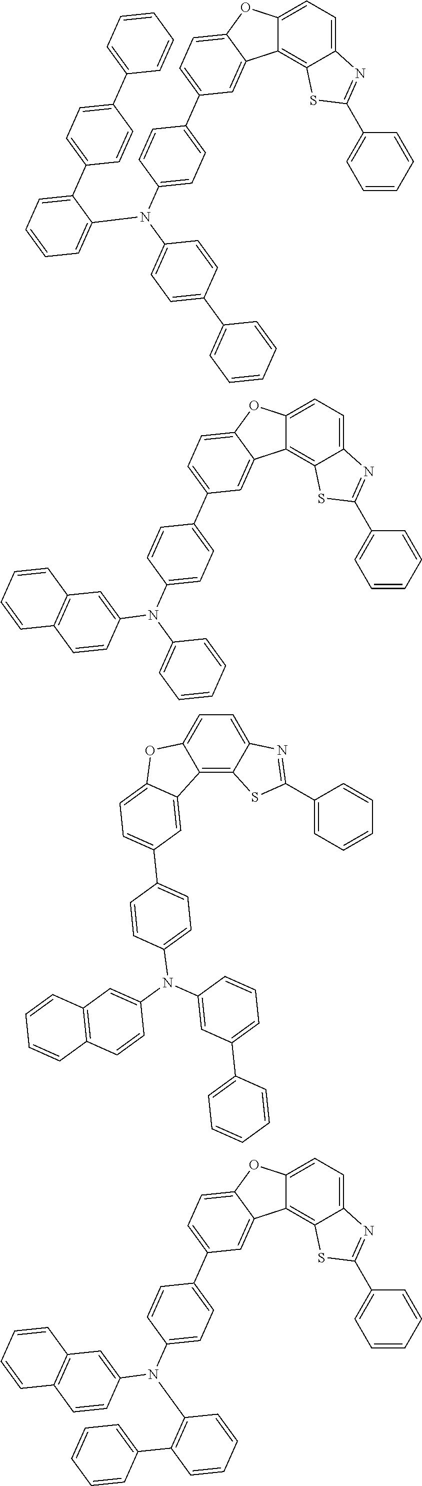

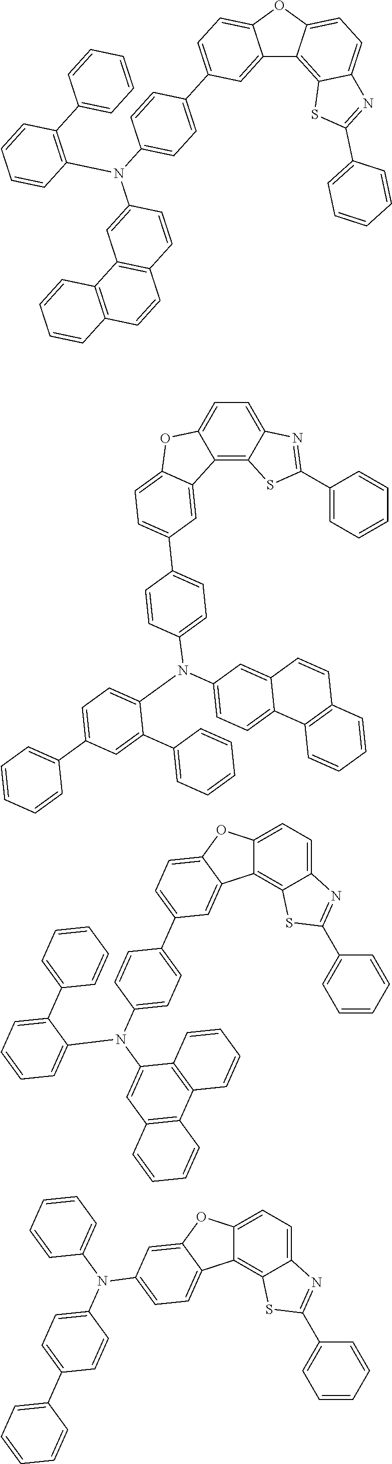

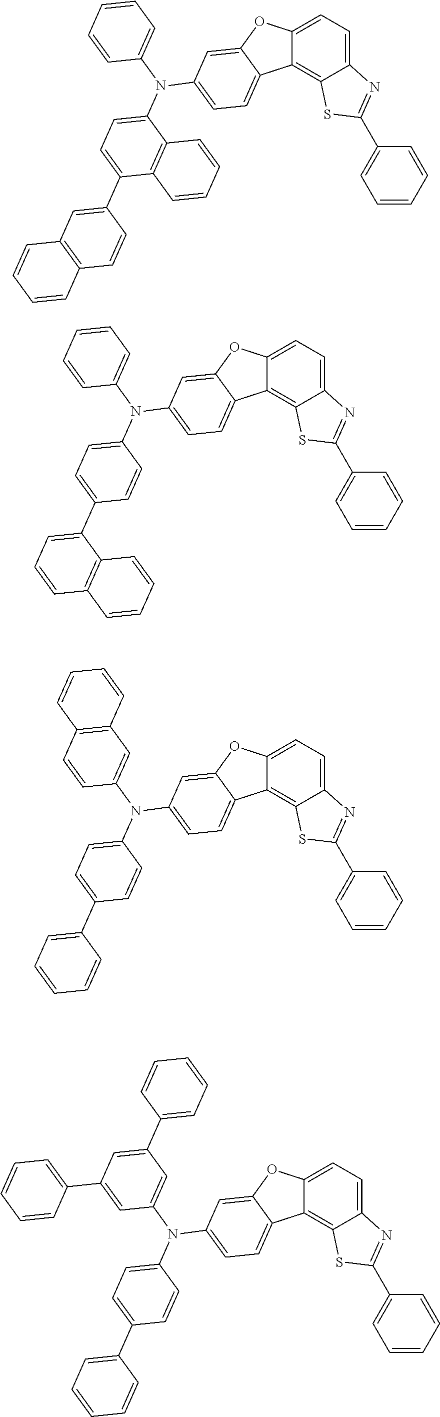

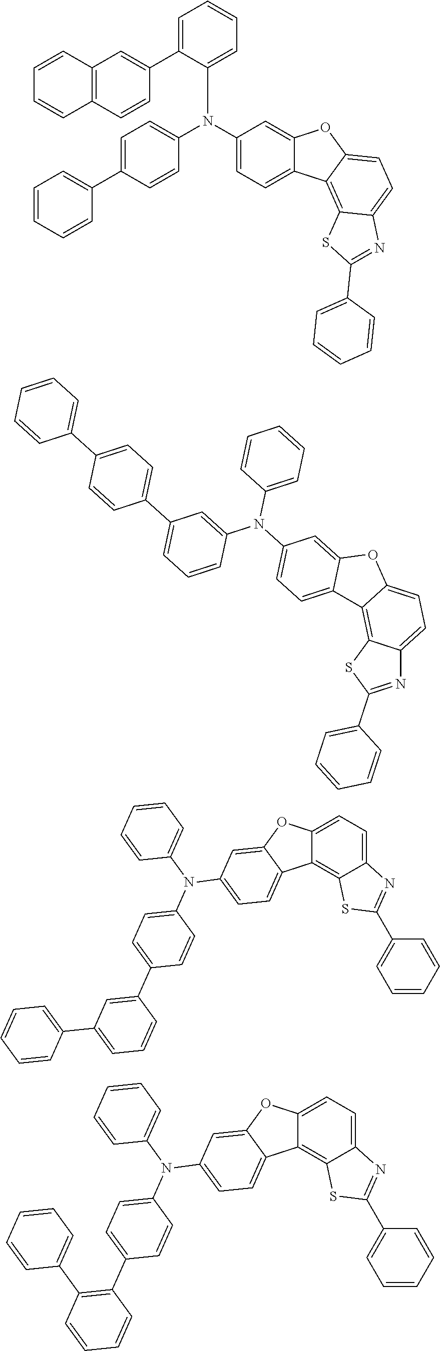

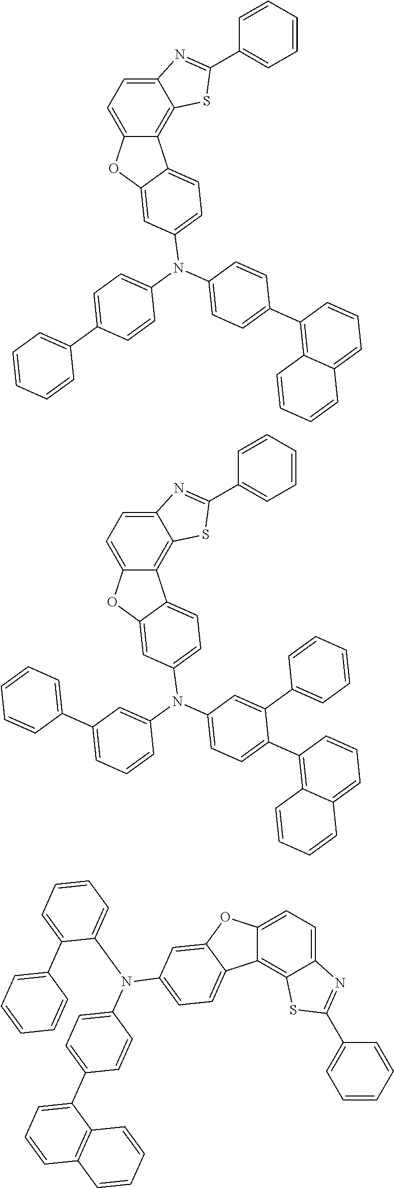

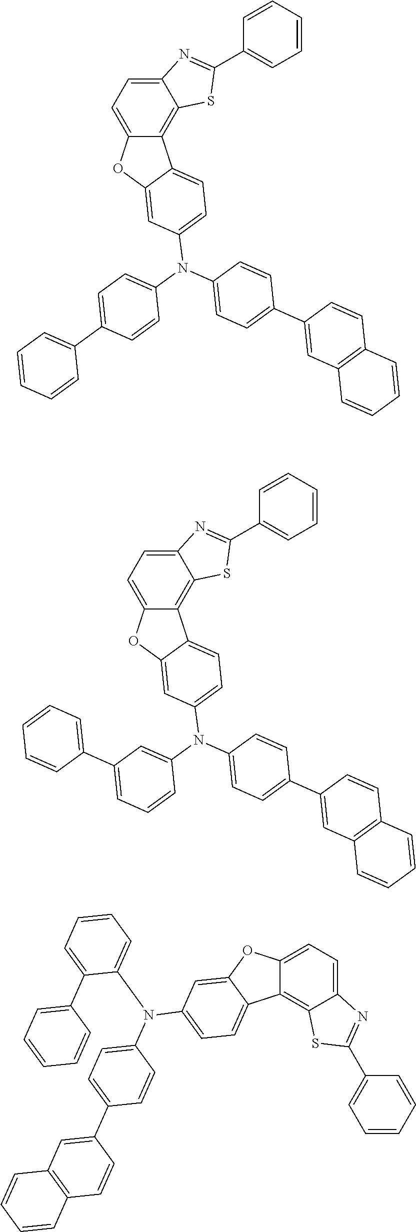 [SMILES: c1ccc(-c2ccc(N(c3ccc(-c4ccc5ccccc5c4)cc3)c3ccc4c(c3)oc3ccc5nc(-c6ccccc6)sc5c34)cc2)cc1 ; c1ccc(-c2cccc(N(c3ccc(-c4ccc5ccccc5c4)cc3)c3ccc4c(c3)oc3ccc5nc(-c6ccccc6)sc5c34)c2)cc1 ; c1ccc(-c2nc3ccc4oc5cc(N(c6ccc(-c7ccc8ccccc8c7)cc6)c6ccccc6-c6ccccc6)ccc5c4c3s2)cc1]

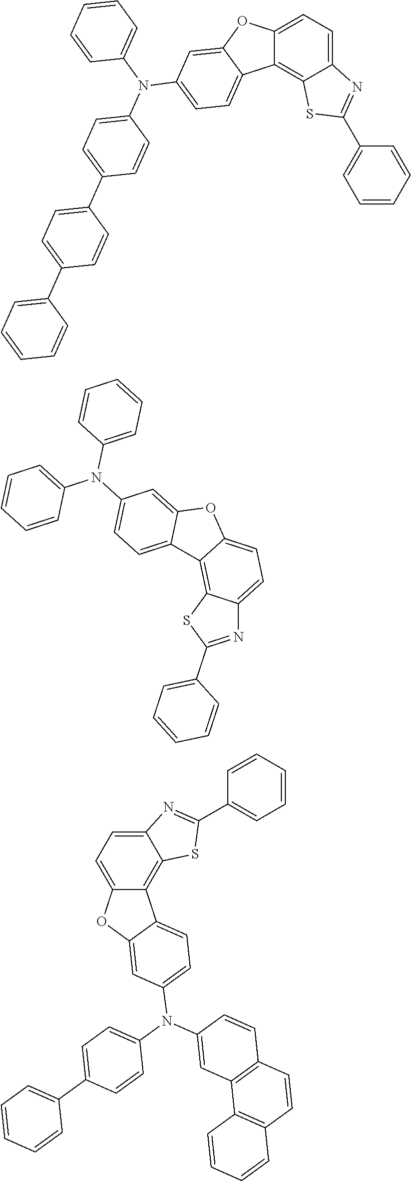

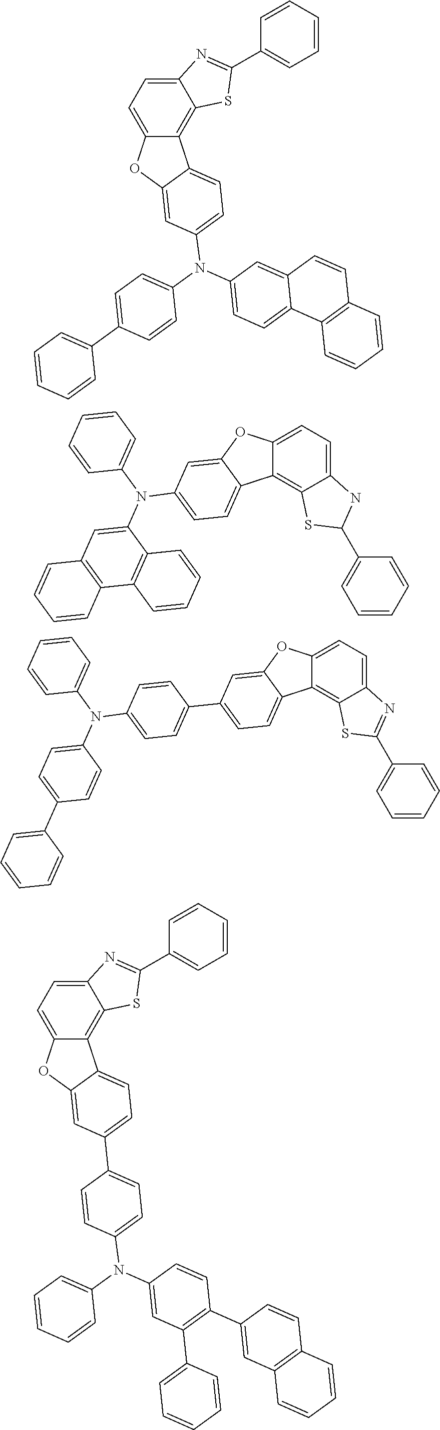

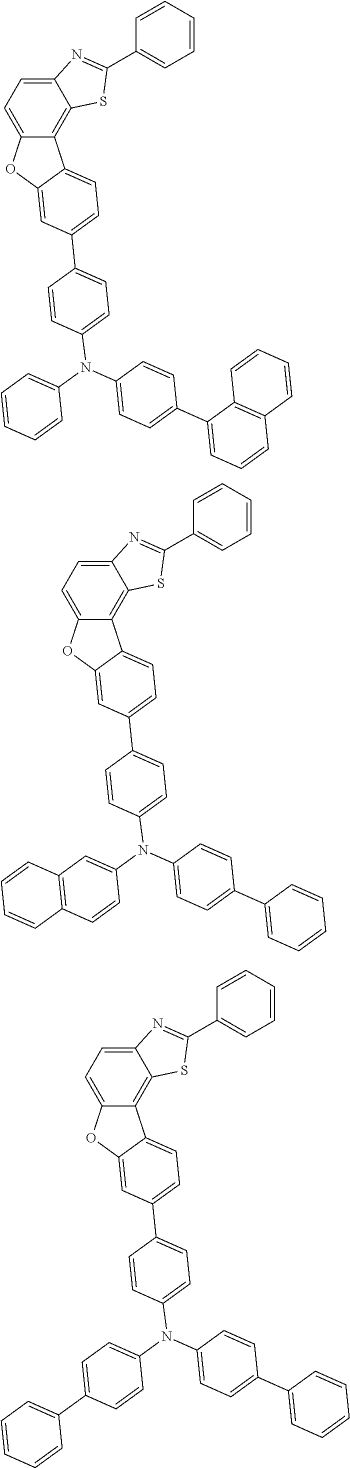

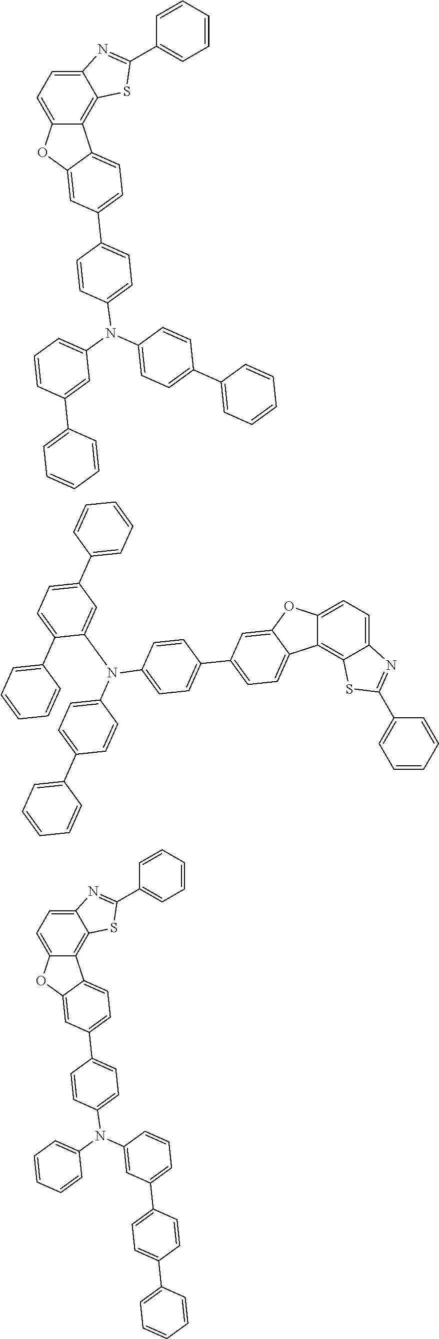

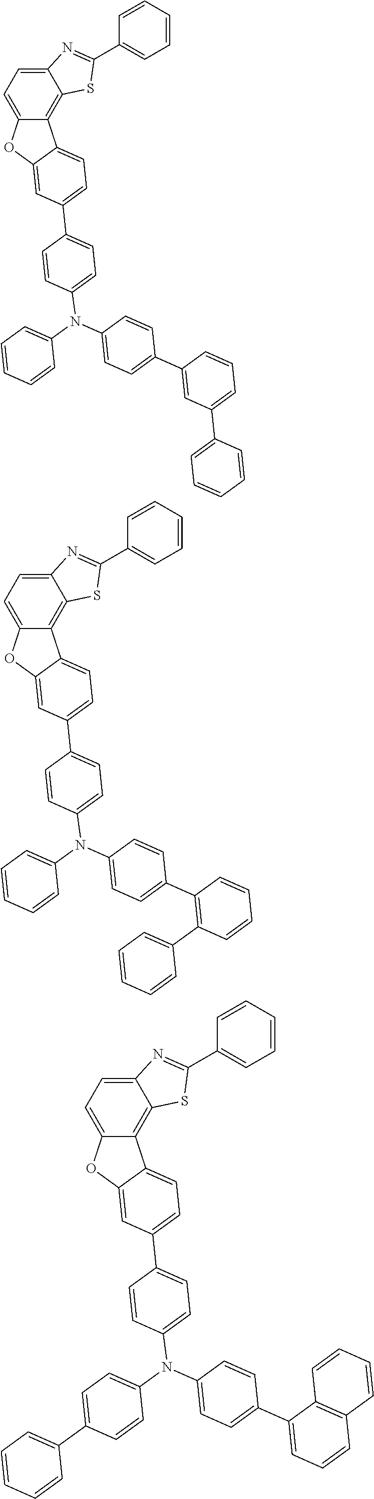

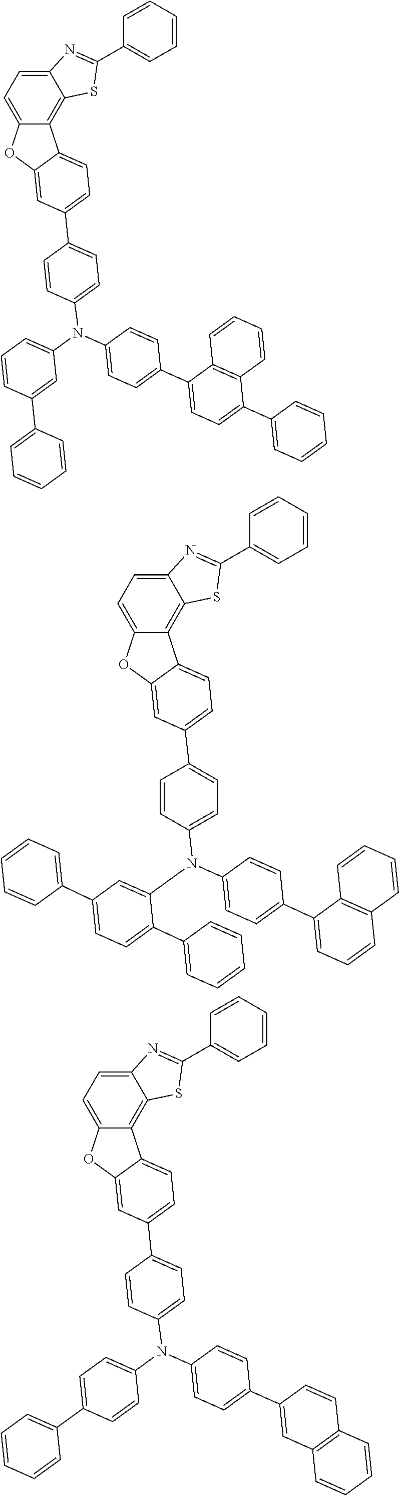

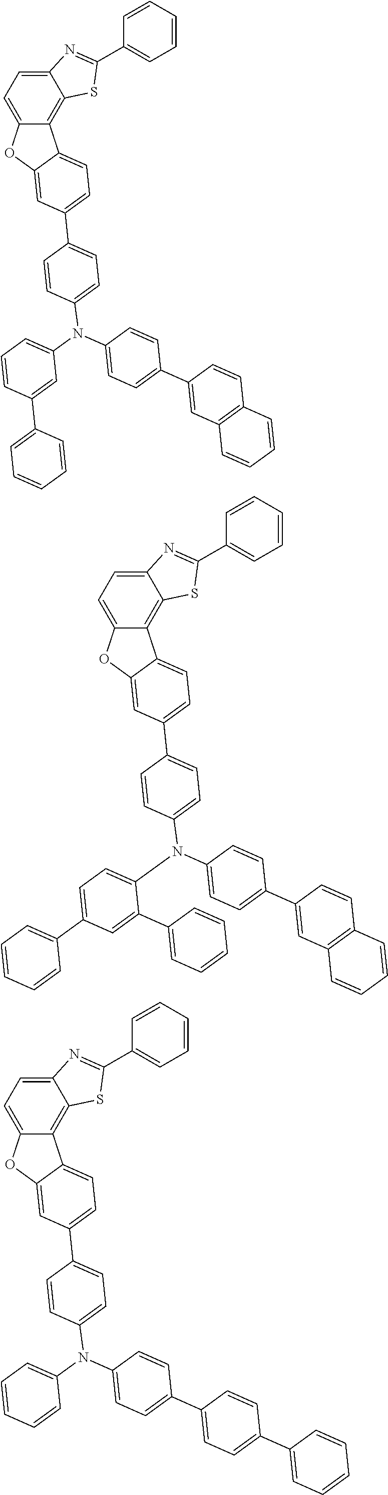

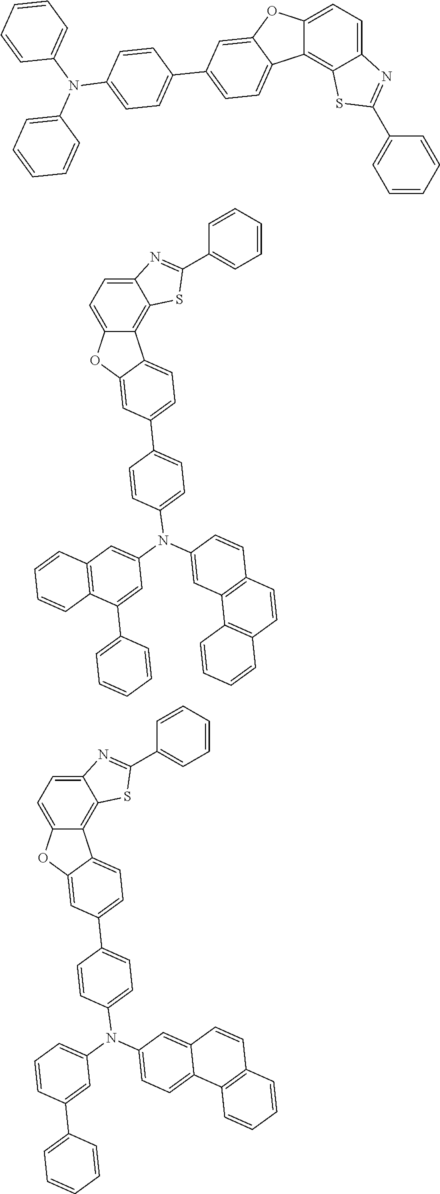

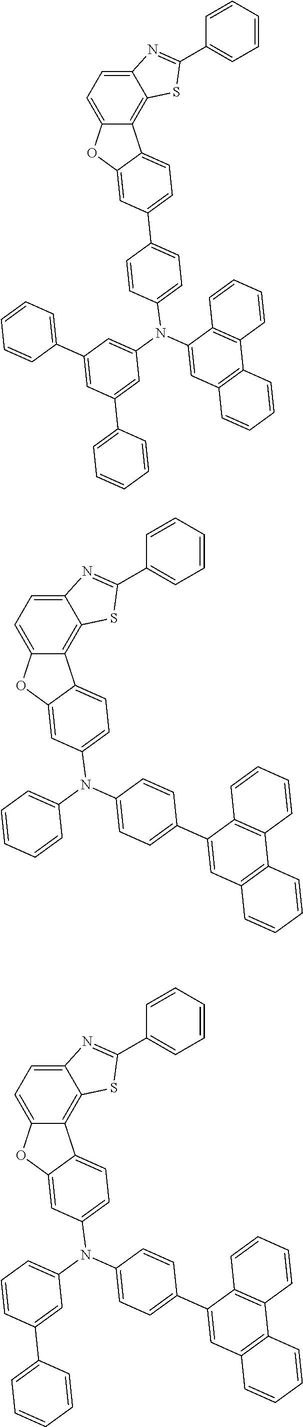 [SMILES: c1ccc(-c2cc(-c3ccccc3)cc(N(c3ccc(-c4ccc5c(c4)oc4ccc6nc(-c7ccccc7)sc6c45)cc3)c3cc4ccccc4c4ccccc34)c2)cc1 ; c1ccc(-c2nc3ccc4oc5cc(N(c6ccccc6)c6ccc(-c7cc8ccccc8c8ccccc78)cc6)ccc5c4c3s2)cc1 ; c1ccc(-c2cccc(N(c3ccc(-c4cc5ccccc5c5ccccc45)cc3)c3ccc4c(c3)oc3ccc5nc(-c6ccccc6)sc5c34)c2)cc1]

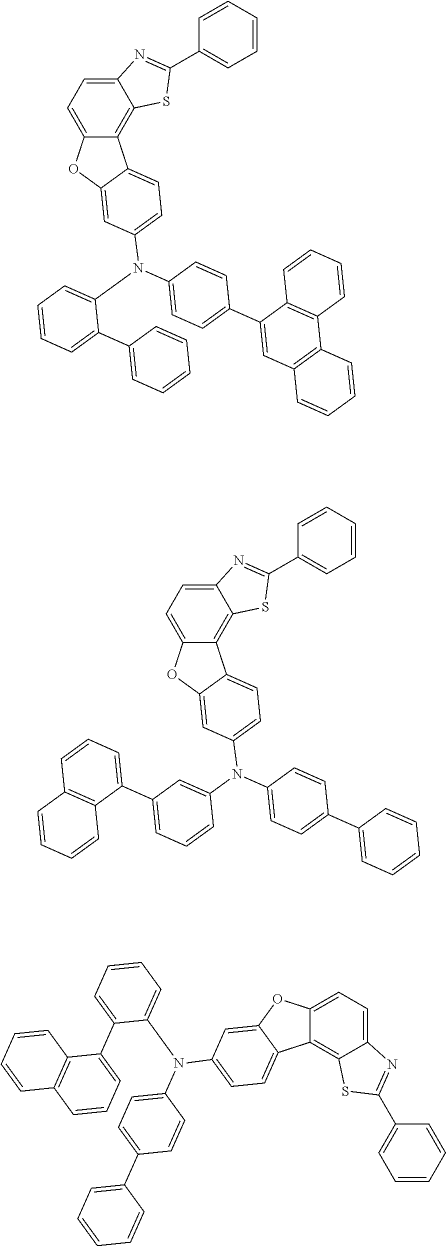 [SMILES: c1ccc(-c2nc3ccc4oc5cc(N(c6ccc(-c7cc8ccccc8c8ccccc78)cc6)c6ccccc6-c6ccccc6)ccc5c4c3s2)cc1 ; c1ccc(-c2ccc(N(c3cccc(-c4cccc5ccccc45)c3)c3ccc4c(c3)oc3ccc5nc(-c6ccccc6)sc5c34)cc2)cc1 ; c1ccc(-c2ccc(N(c3ccc4c(c3)oc3ccc5nc(-c6ccccc6)sc5c34)c3ccccc3-c3cccc4ccccc34)cc2)cc1]

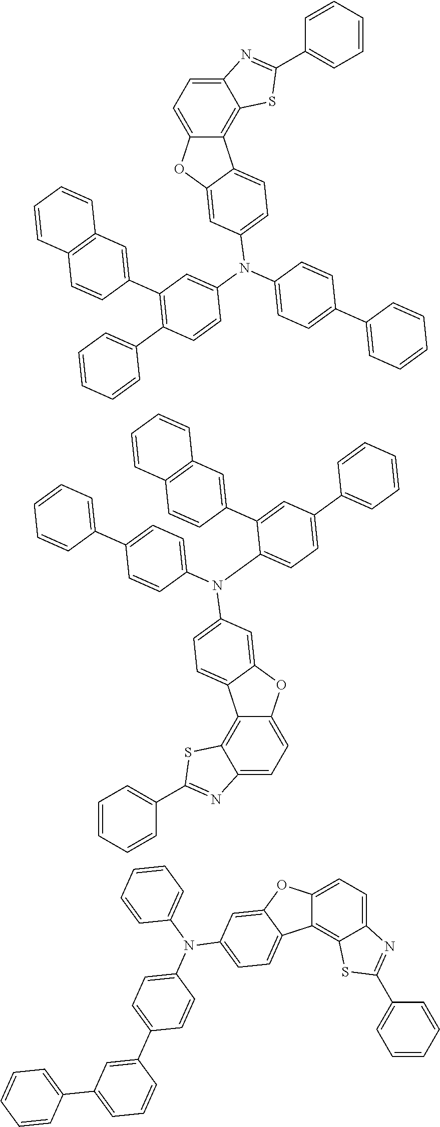

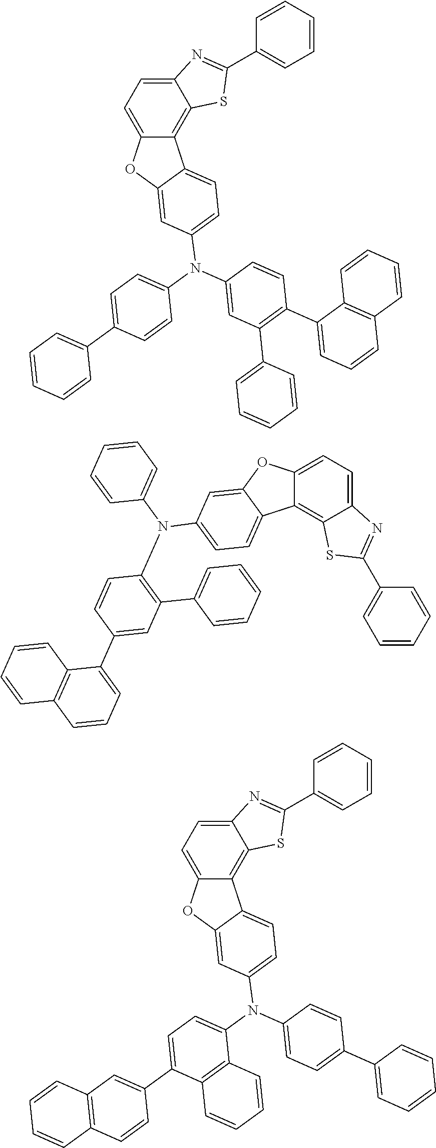

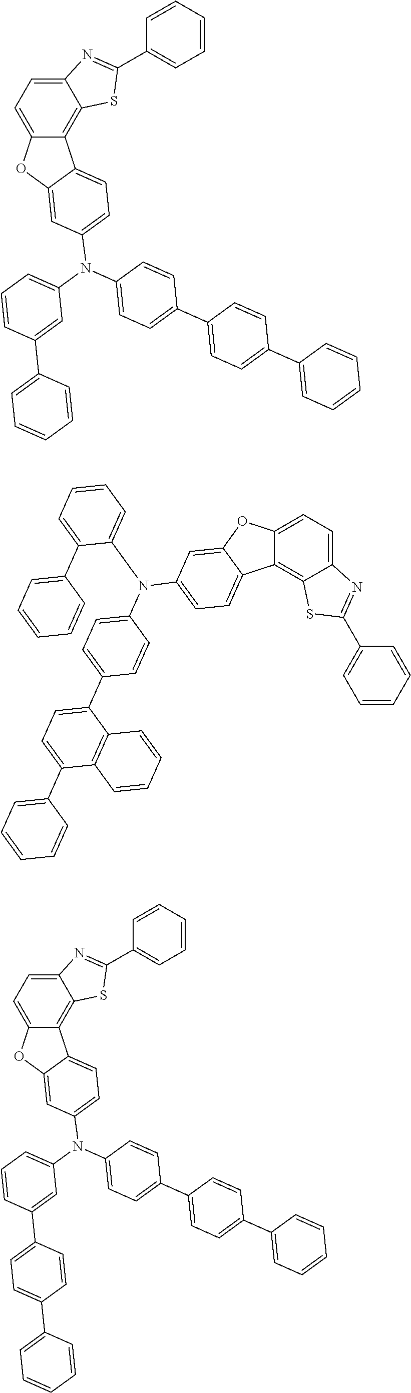 [SMILES: c1ccc(-c2ccc(-c3ccc(N(c4cccc(-c5ccccc5)c4)c4ccc5c(c4)oc4ccc6nc(-c7ccccc7)sc6c45)cc3)cc2)cc1 ; c1ccc(-c2nc3ccc4oc5cc(N(c6ccc(-c7ccc(-c8ccccc8)c8ccccc78)cc6)c6ccccc6-c6ccccc6)ccc5c4c3s2)cc1 ; c1ccc(-c2ccc(-c3ccc(N(c4cccc(-c5ccc(-c6ccccc6)cc5)c4)c4ccc5c(c4)oc4ccc6nc(-c7ccccc7)sc6c45)cc3)cc2)cc1]

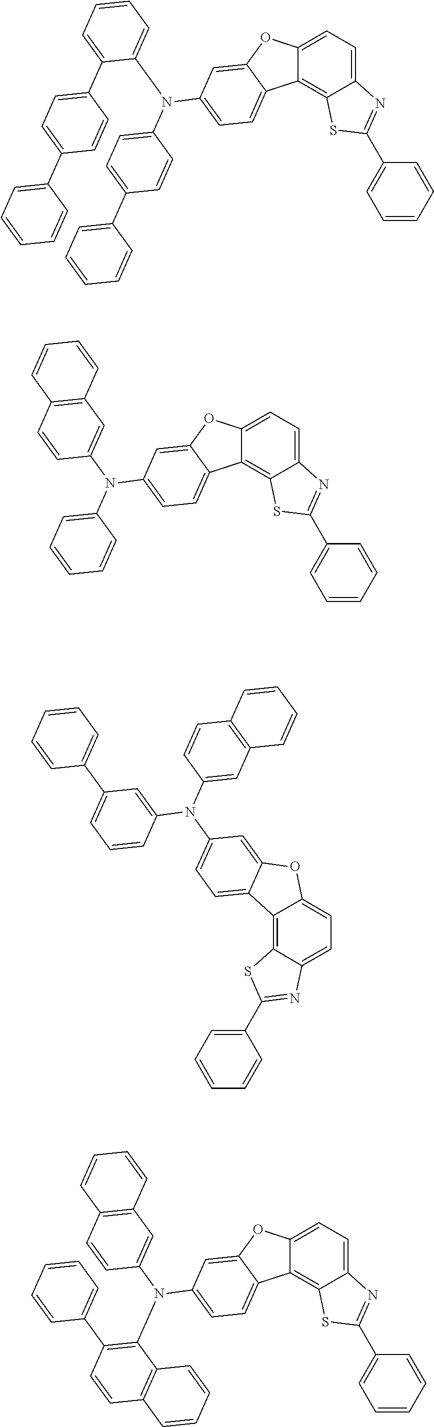 [SMILES: c1ccc(-c2ccc(-c3ccccc3N(c3ccc(-c4ccccc4)cc3)c3ccc4c(c3)oc3ccc5nc(-c6ccccc6)sc5c34)cc2)cc1 ; c1ccc(-c2nc3ccc4oc5cc(N(c6ccccc6)c6ccc7ccccc7c6)ccc5c4c3s2)cc1 ; c1ccc(-c2cccc(N(c3ccc4ccccc4c3)c3ccc4c(c3)oc3ccc5nc(-c6ccccc6)sc5c34)c2)cc1 ; c1ccc(-c2nc3ccc4oc5cc(N(c6ccc7ccccc7c6)c6c(-c7ccccc7)ccc7ccccc67)ccc5c4c3s2)cc1]

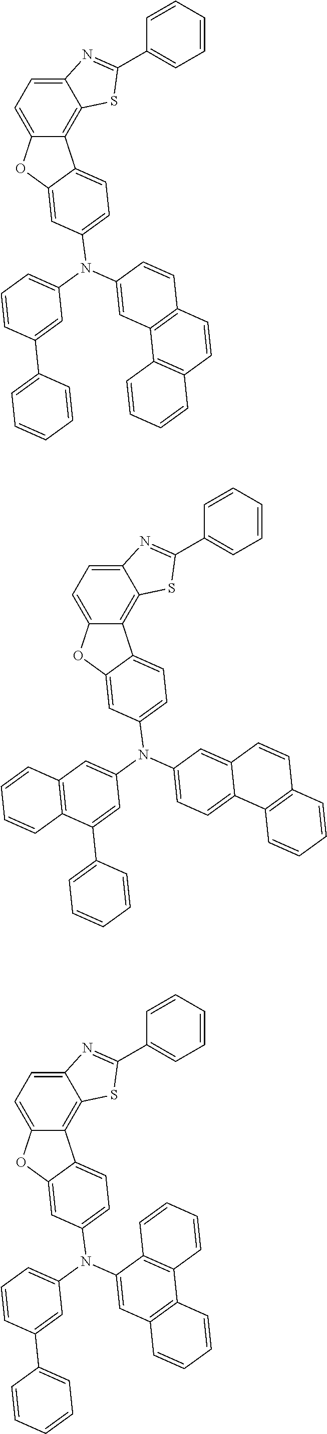 [SMILES: c1ccc(-c2cccc(N(c3ccc4c(c3)oc3ccc5nc(-c6ccccc6)sc5c34)c3ccc4ccc5ccccc5c4c3)c2)cc1 ; c1ccc(-c2nc3ccc4oc5cc(N(c6cc(-c7ccccc7)c7ccccc7c6)c6ccc7c(ccc8ccccc87)c6)ccc5c4c3s2)cc1 ; c1ccc(-c2cccc(N(c3ccc4c(c3)oc3ccc5nc(-c6ccccc6)sc5c34)c3cc4ccccc4c4ccccc34)c2)cc1]

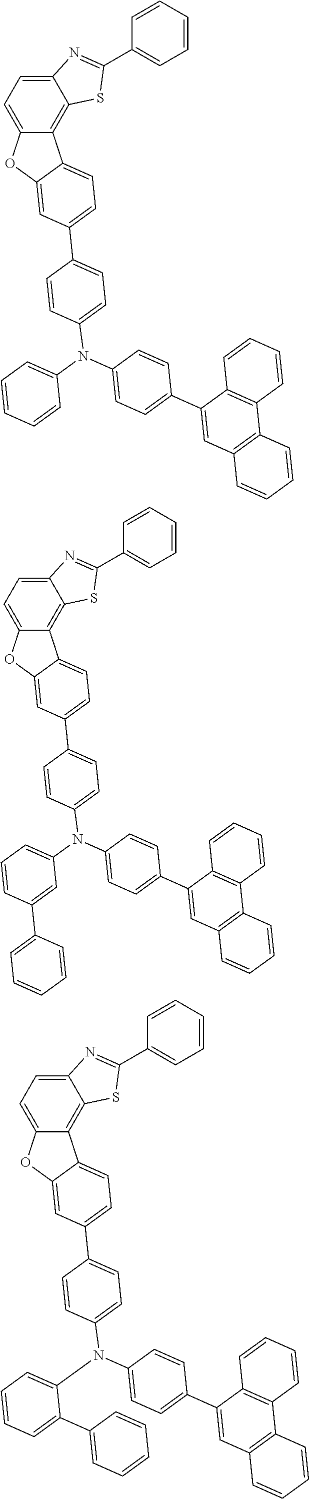 [SMILES: c1ccc(-c2nc3ccc4oc5cc(-c6ccc(N(c7ccccc7)c7ccc(-c8cc9ccccc9c9ccccc89)cc7)cc6)ccc5c4c3s2)cc1 ; c1ccc(-c2cccc(N(c3ccc(-c4ccc5c(c4)oc4ccc6nc(-c7ccccc7)sc6c45)cc3)c3ccc(-c4cc5ccccc5c5ccccc45)cc3)c2)cc1 ; c1ccc(-c2nc3ccc4oc5cc(-c6ccc(N(c7ccc(-c8cc9ccccc9c9ccccc89)cc7)c7ccccc7-c7ccccc7)cc6)ccc5c4c3s2)cc1]

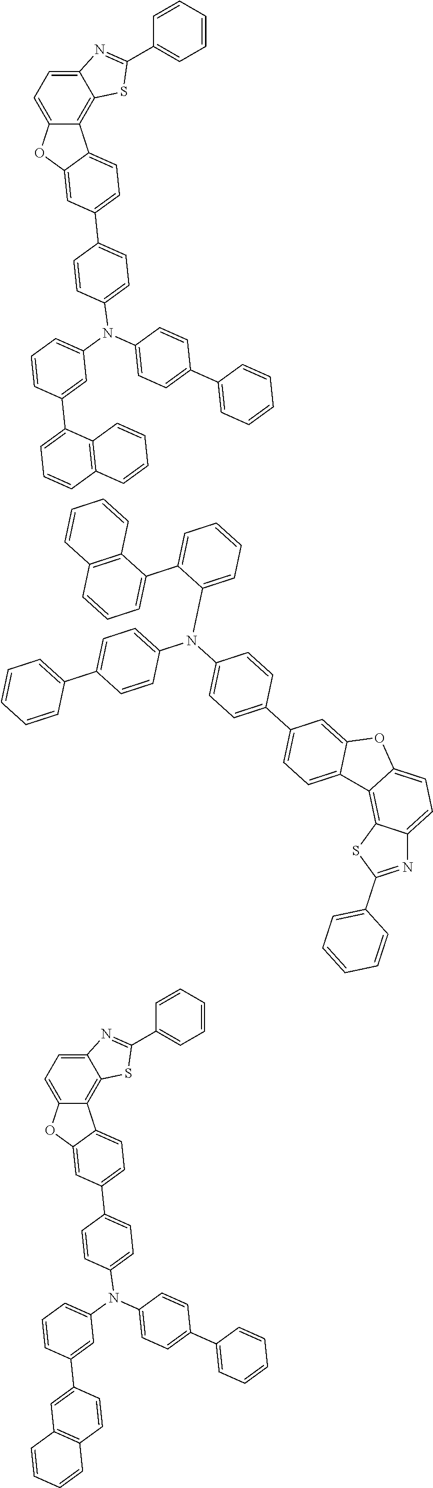

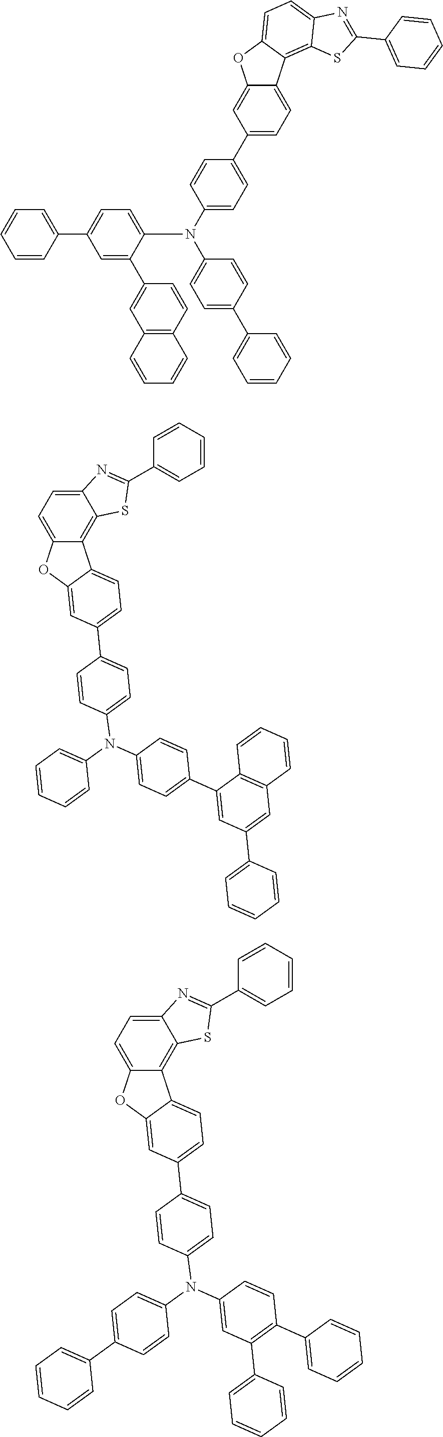

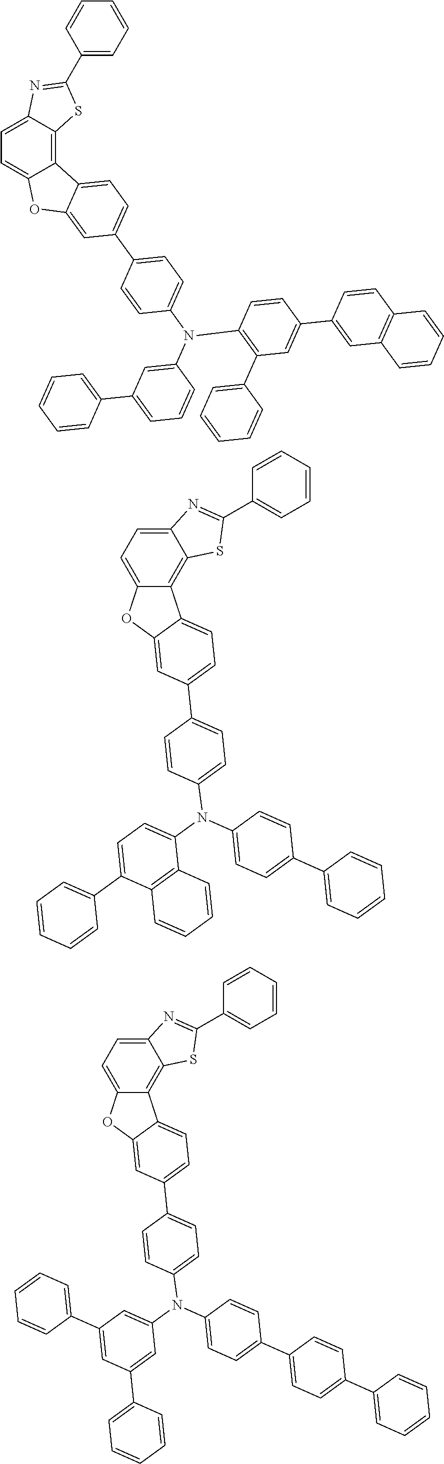

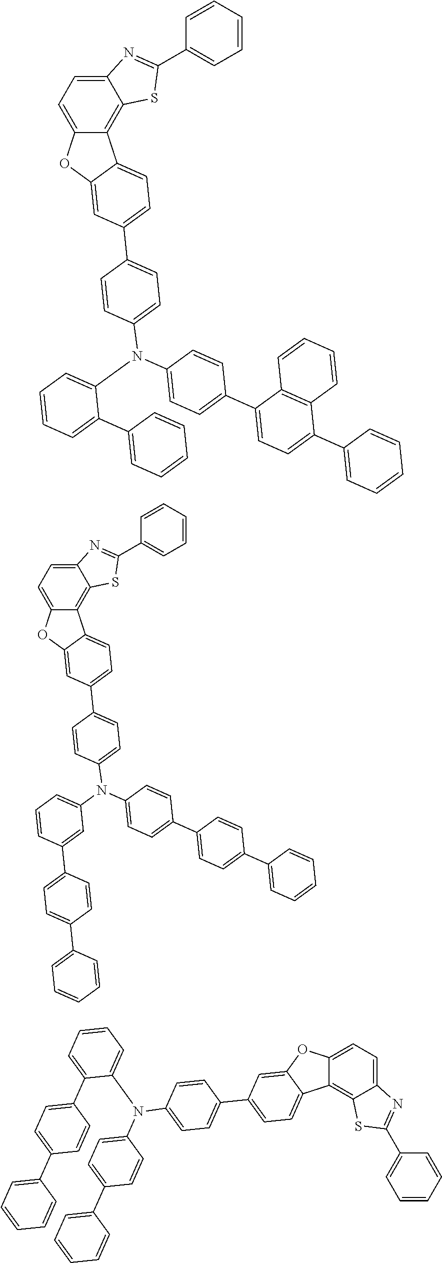

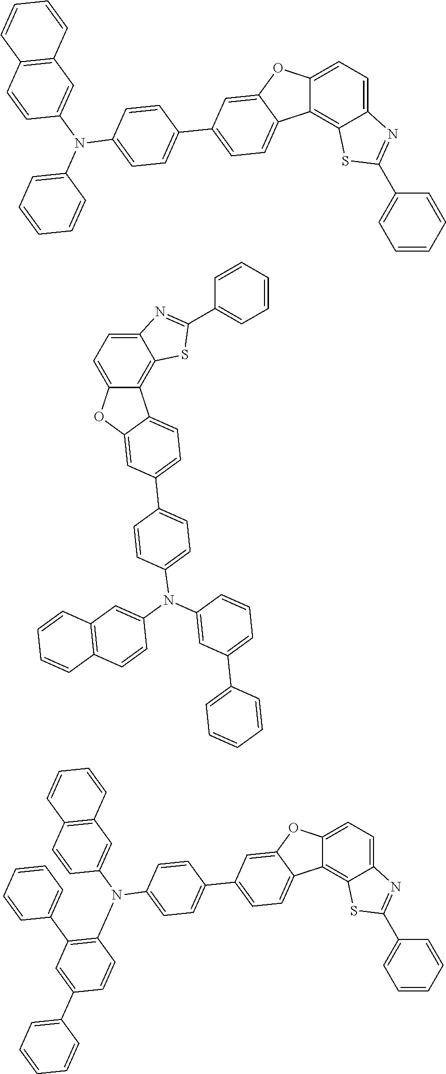 [SMILES: c1ccc(-c2nc3ccc4oc5cc(-c6ccc(N(c7ccccc7)c7ccc8ccccc8c7)cc6)ccc5c4c3s2)cc1 ; c1ccc(-c2cccc(N(c3ccc(-c4ccc5c(c4)oc4ccc6nc(-c7ccccc7)sc6c45)cc3)c3ccc4ccccc4c3)c2)cc1 ; c1ccc(-c2ccc(N(c3ccc(-c4ccc5c(c4)oc4ccc6nc(-c7ccccc7)sc6c45)cc3)c3ccc4ccccc4c3)c(-c3ccccc3)c2)cc1]

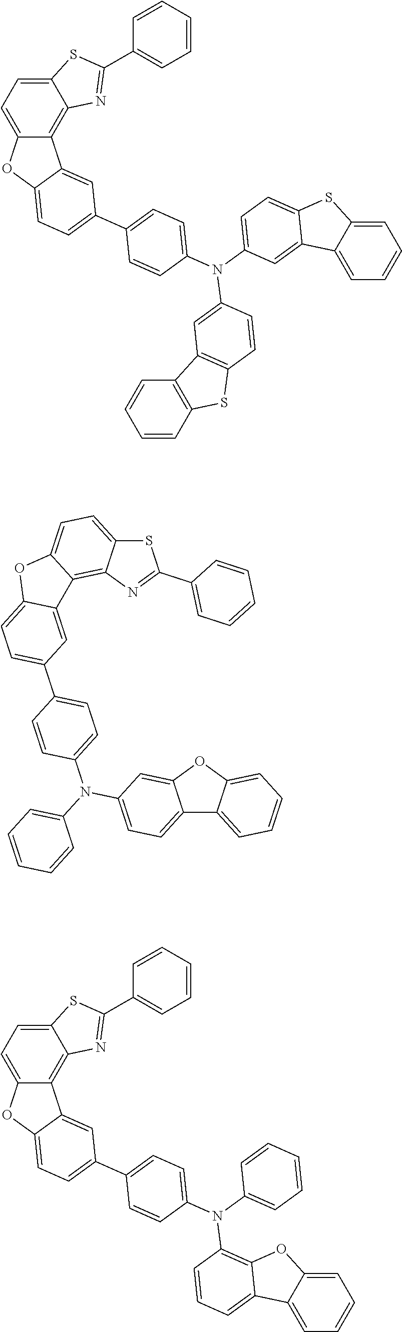 [SMILES: c1ccc(-c2nc3c(ccc4oc5ccc(-c6ccc(N(c7ccc8sc9ccccc9c8c7)c7ccc8sc9ccccc9c8c7)cc6)cc5c43)s2)cc1 ; c1ccc(-c2nc3c(ccc4oc5ccc(-c6ccc(N(c7ccccc7)c7ccc8c(c7)oc7ccccc78)cc6)cc5c43)s2)cc1 ; c1ccc(-c2nc3c(ccc4oc5ccc(-c6ccc(N(c7ccccc7)c7cccc8c7oc7ccccc78)cc6)cc5c43)s2)cc1]

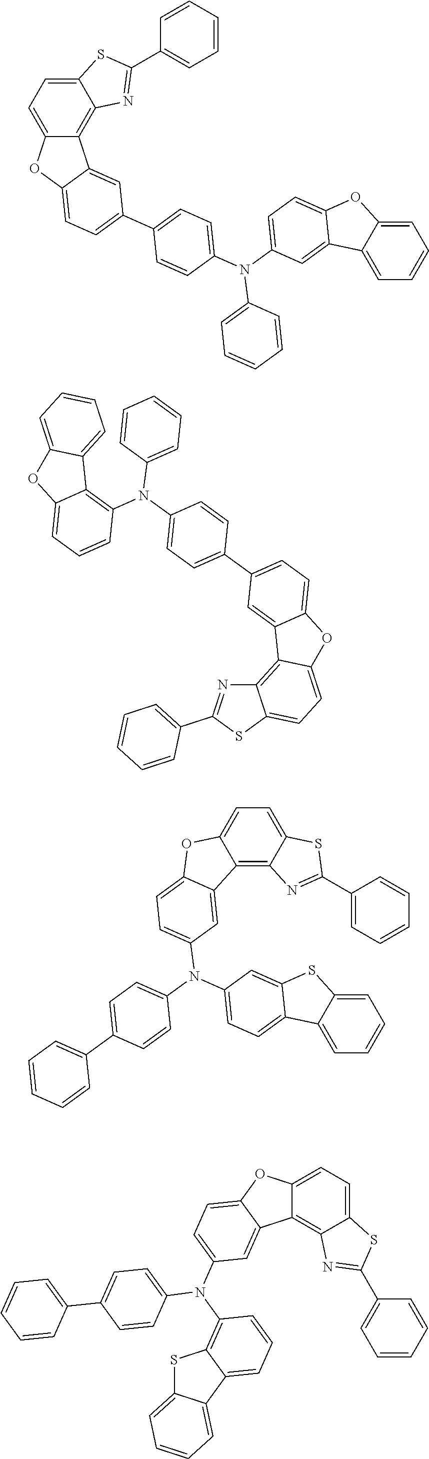 [SMILES: c1ccc(-c2nc3c(ccc4oc5ccc(-c6ccc(N(c7ccccc7)c7ccc8oc9ccccc9c8c7)cc6)cc5c43)s2)cc1 ; c1ccc(-c2nc3c(ccc4oc5ccc(-c6ccc(N(c7ccccc7)c7cccc8oc9ccccc9c78)cc6)cc5c43)s2)cc1 ; c1ccc(-c2ccc(N(c3ccc4c(c3)sc3ccccc34)c3ccc4oc5ccc6sc(-c7ccccc7)nc6c5c4c3)cc2)cc1 ; c1ccc(-c2ccc(N(c3ccc4oc5ccc6sc(-c7ccccc7)nc6c5c4c3)c3cccc4c3sc3ccccc34)cc2)cc1]

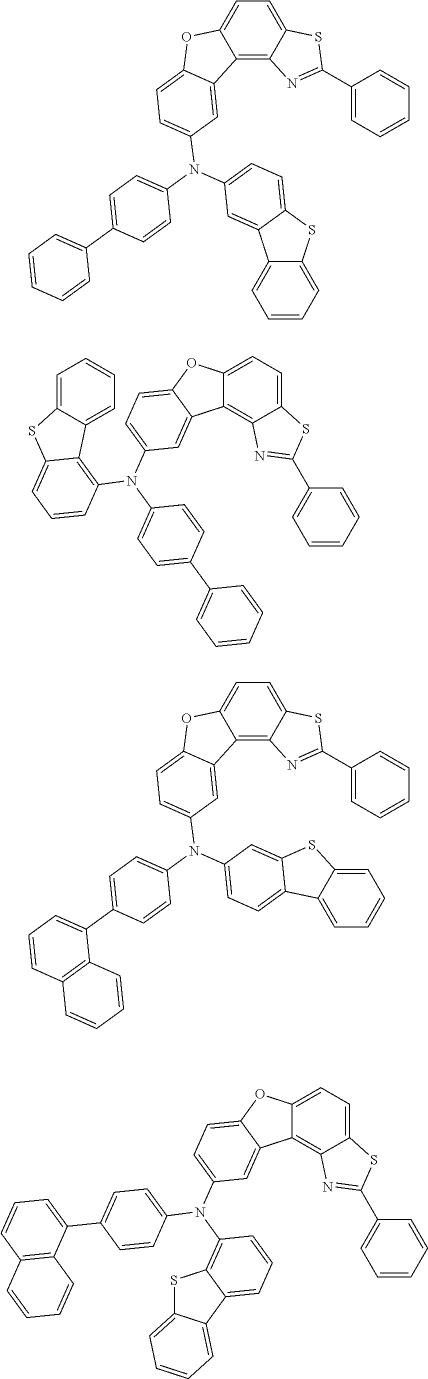

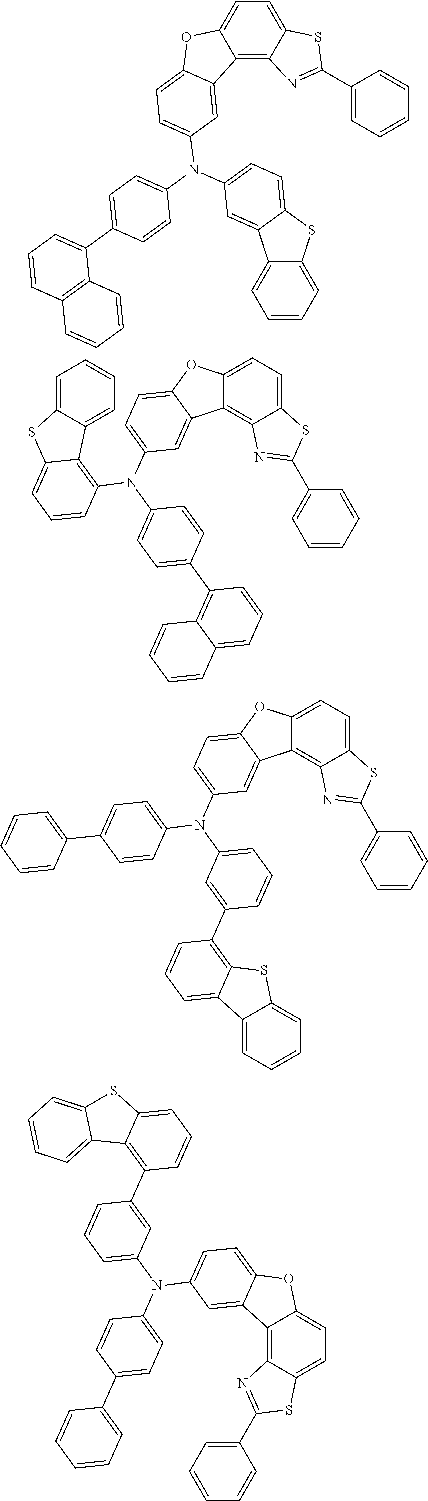

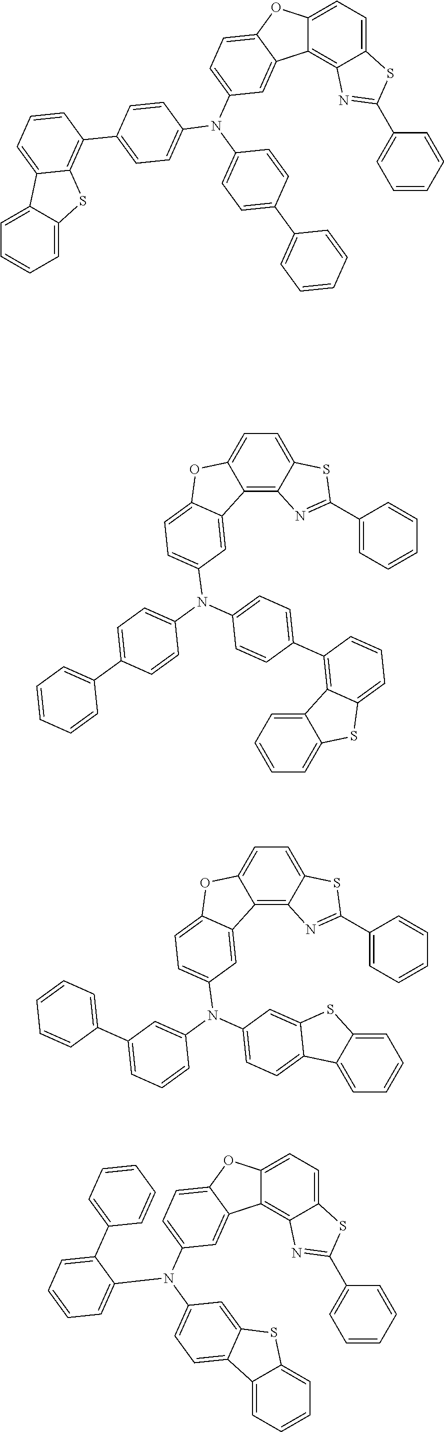 [SMILES: c1ccc(-c2ccc(N(c3ccc(-c4cccc5c4sc4ccccc45)cc3)c3ccc4oc5ccc6sc(-c7ccccc7)nc6c5c4c3)cc2)cc1 ; c1ccc(-c2ccc(N(c3ccc(-c4cccc5sc6ccccc6c45)cc3)c3ccc4oc5ccc6sc(-c7ccccc7)nc6c5c4c3)cc2)cc1 ; c1ccc(-c2cccc(N(c3ccc4c(c3)sc3ccccc34)c3ccc4oc5ccc6sc(-c7ccccc7)nc6c5c4c3)c2)cc1 ; c1ccc(-c2nc3c(ccc4oc5ccc(N(c6ccc7c(c6)sc6ccccc67)c6ccccc6-c6ccccc6)cc5c43)s2)cc1]

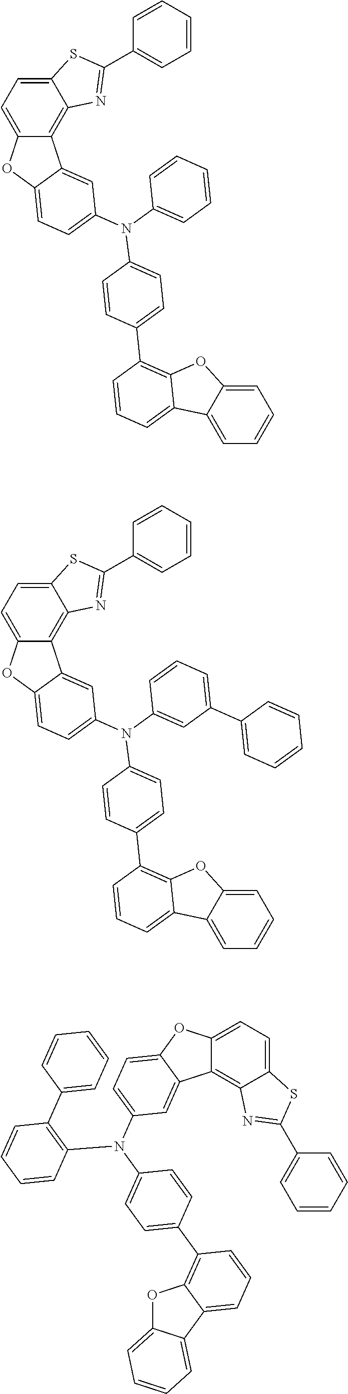 [SMILES: c1ccc(-c2nc3c(ccc4oc5ccc(N(c6ccccc6)c6ccc(-c7cccc8c7oc7ccccc78)cc6)cc5c43)s2)cc1 ; c1ccc(-c2cccc(N(c3ccc(-c4cccc5c4oc4ccccc45)cc3)c3ccc4oc5ccc6sc(-c7ccccc7)nc6c5c4c3)c2)cc1 ; c1ccc(-c2nc3c(ccc4oc5ccc(N(c6ccc(-c7cccc8c7oc7ccccc78)cc6)c6ccccc6-c6ccccc6)cc5c43)s2)cc1]

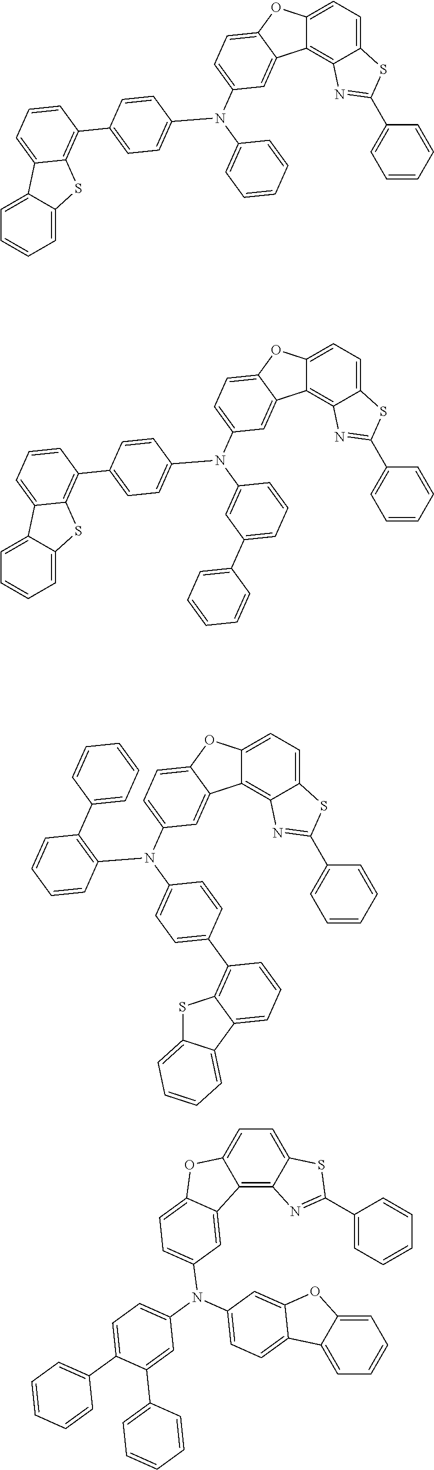 [SMILES: c1ccc(-c2nc3c(ccc4oc5ccc(N(c6ccccc6)c6ccc(-c7cccc8c7sc7ccccc78)cc6)cc5c43)s2)cc1 ; c1ccc(-c2cccc(N(c3ccc(-c4cccc5c4sc4ccccc45)cc3)c3ccc4oc5ccc6sc(-c7ccccc7)nc6c5c4c3)c2)cc1 ; c1ccc(-c2nc3c(ccc4oc5ccc(N(c6ccc(-c7cccc8c7sc7ccccc78)cc6)c6ccccc6-c6ccccc6)cc5c43)s2)cc1 ; c1ccc(-c2nc3c(ccc4oc5ccc(N(c6ccc(-c7ccccc7)c(-c7ccccc7)c6)c6ccc7c(c6)oc6ccccc67)cc5c43)s2)cc1]

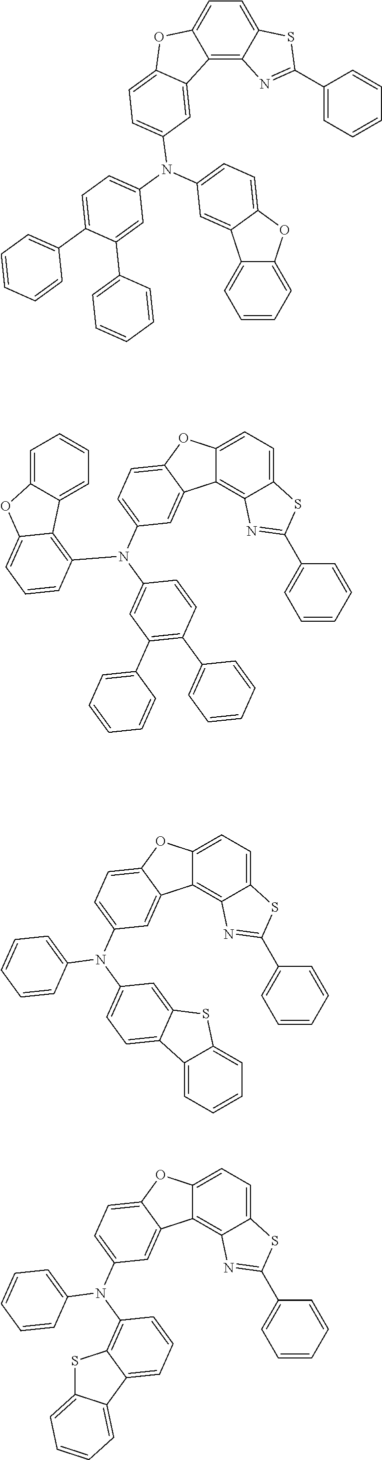 [SMILES: c1ccc(-c2nc3c(ccc4oc5ccc(N(c6ccc(-c7ccccc7)c(-c7ccccc7)c6)c6ccc7oc8ccccc8c7c6)cc5c43)s2)cc1 ; c1ccc(-c2nc3c(ccc4oc5ccc(N(c6ccc(-c7ccccc7)c(-c7ccccc7)c6)c6cccc7oc8ccccc8c67)cc5c43)s2)cc1 ; c1ccc(-c2nc3c(ccc4oc5ccc(N(c6ccccc6)c6ccc7c(c6)sc6ccccc67)cc5c43)s2)cc1 ; c1ccc(-c2nc3c(ccc4oc5ccc(N(c6ccccc6)c6cccc7c6sc6ccccc67)cc5c43)s2)cc1]

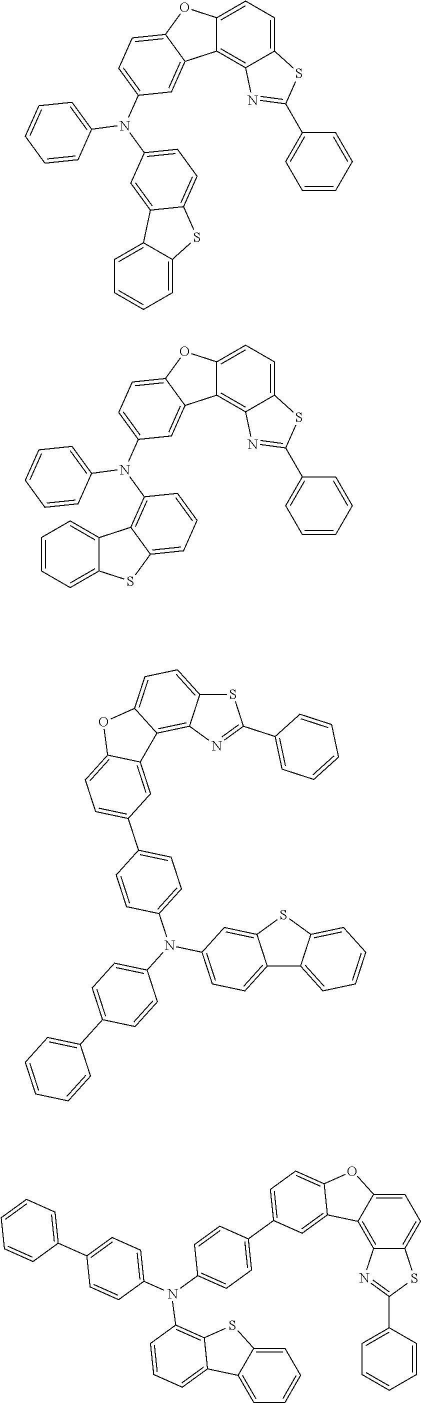 [SMILES: c1ccc(-c2nc3c(ccc4oc5ccc(N(c6ccccc6)c6ccc7sc8ccccc8c7c6)cc5c43)s2)cc1 ; c1ccc(-c2nc3c(ccc4oc5ccc(N(c6ccccc6)c6cccc7sc8ccccc8c67)cc5c43)s2)cc1 ; c1ccc(-c2ccc(N(c3ccc(-c4ccc5oc6ccc7sc(-c8ccccc8)nc7c6c5c4)cc3)c3ccc4c(c3)sc3ccccc34)cc2)cc1 ; c1ccc(-c2ccc(N(c3ccc(-c4ccc5oc6ccc7sc(-c8ccccc8)nc7c6c5c4)cc3)c3cccc4c3sc3ccccc34)cc2)cc1]

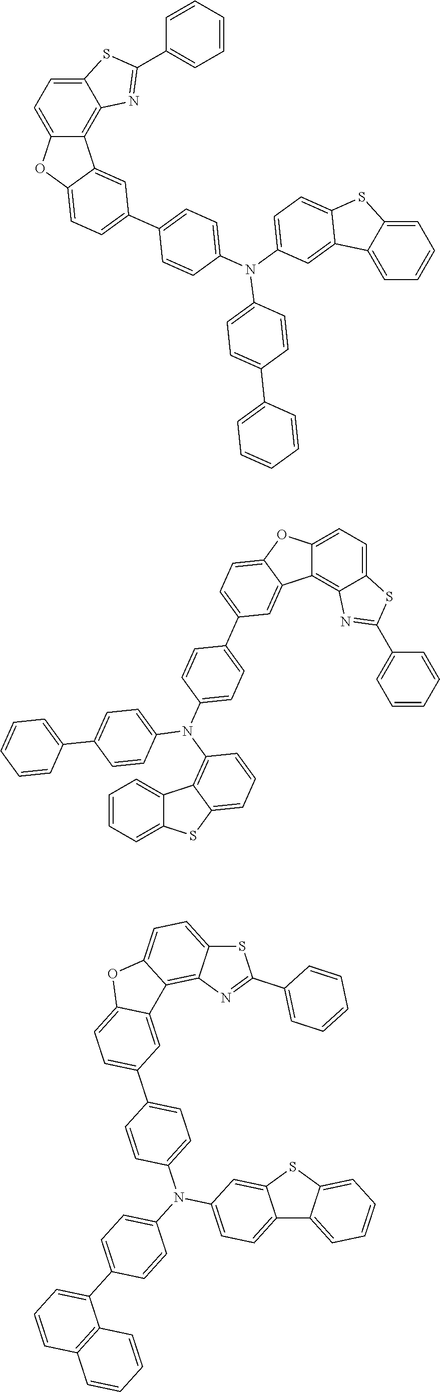 [SMILES: c1ccc(-c2ccc(N(c3ccc(-c4ccc5oc6ccc7sc(-c8ccccc8)nc7c6c5c4)cc3)c3ccc4sc5ccccc5c4c3)cc2)cc1 ; c1ccc(-c2ccc(N(c3ccc(-c4ccc5oc6ccc7sc(-c8ccccc8)nc7c6c5c4)cc3)c3cccc4sc5ccccc5c34)cc2)cc1 ; c1ccc(-c2nc3c(ccc4oc5ccc(-c6ccc(N(c7ccc(-c8cccc9ccccc89)cc7)c7ccc8c(c7)sc7ccccc78)cc6)cc5c43)s2)cc1]

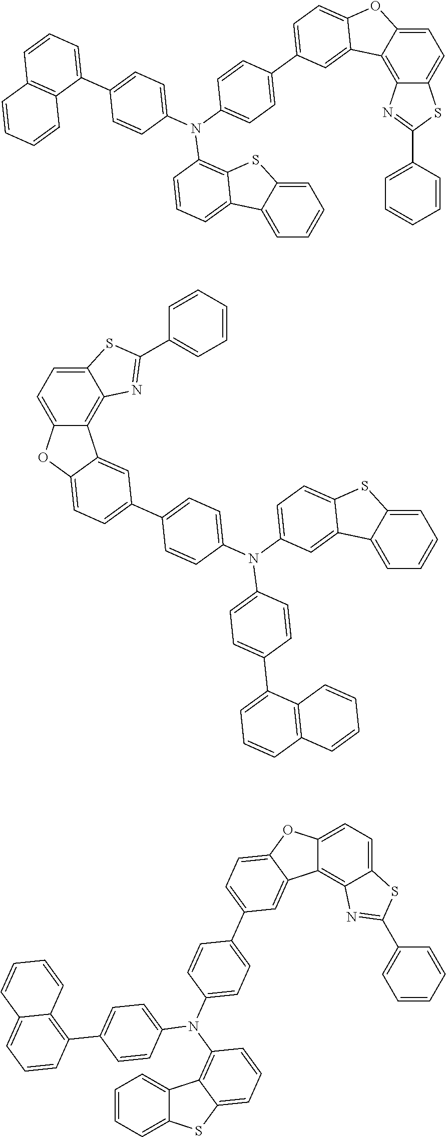 [SMILES: c1ccc(-c2nc3c(ccc4oc5ccc(-c6ccc(N(c7ccc(-c8cccc9ccccc89)cc7)c7cccc8c7sc7ccccc78)cc6)cc5c43)s2)cc1 ; c1ccc(-c2nc3c(ccc4oc5ccc(-c6ccc(N(c7ccc(-c8cccc9ccccc89)cc7)c7ccc8sc9ccccc9c8c7)cc6)cc5c43)s2)cc1 ; c1ccc(-c2nc3c(ccc4oc5ccc(-c6ccc(N(c7ccc(-c8cccc9ccccc89)cc7)c7cccc8sc9ccccc9c78)cc6)cc5c43)s2)cc1]

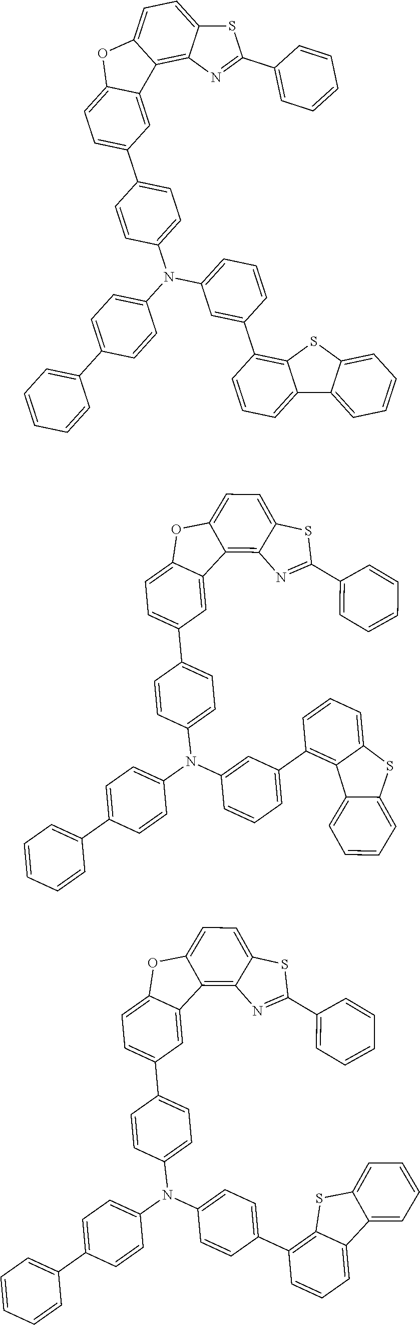 [SMILES: c1ccc(-c2ccc(N(c3ccc(-c4ccc5oc6ccc7sc(-c8ccccc8)nc7c6c5c4)cc3)c3cccc(-c4cccc5c4sc4ccccc45)c3)cc2)cc1 ; c1ccc(-c2ccc(N(c3ccc(-c4ccc5oc6ccc7sc(-c8ccccc8)nc7c6c5c4)cc3)c3cccc(-c4cccc5sc6ccccc6c45)c3)cc2)cc1 ; c1ccc(-c2ccc(N(c3ccc(-c4ccc5oc6ccc7sc(-c8ccccc8)nc7c6c5c4)cc3)c3ccc(-c4cccc5c4sc4ccccc45)cc3)cc2)cc1]

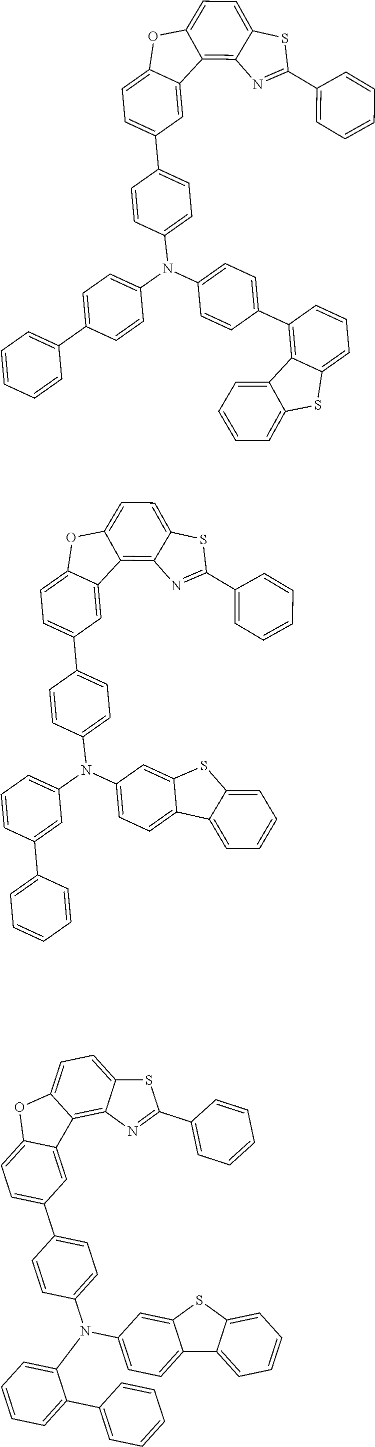 [SMILES: c1ccc(-c2ccc(N(c3ccc(-c4ccc5oc6ccc7sc(-c8ccccc8)nc7c6c5c4)cc3)c3ccc(-c4cccc5sc6ccccc6c45)cc3)cc2)cc1 ; c1ccc(-c2cccc(N(c3ccc(-c4ccc5oc6ccc7sc(-c8ccccc8)nc7c6c5c4)cc3)c3ccc4c(c3)sc3ccccc34)c2)cc1 ; c1ccc(-c2nc3c(ccc4oc5ccc(-c6ccc(N(c7ccc8c(c7)sc7ccccc78)c7ccccc7-c7ccccc7)cc6)cc5c43)s2)cc1]

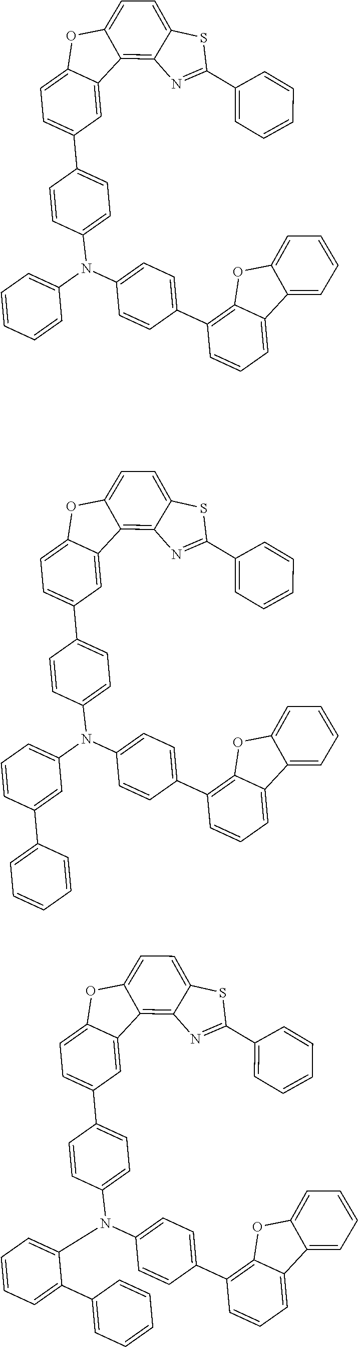 [SMILES: c1ccc(-c2nc3c(ccc4oc5ccc(-c6ccc(N(c7ccccc7)c7ccc(-c8cccc9c8oc8ccccc89)cc7)cc6)cc5c43)s2)cc1 ; c1ccc(-c2cccc(N(c3ccc(-c4ccc5oc6ccc7sc(-c8ccccc8)nc7c6c5c4)cc3)c3ccc(-c4cccc5c4oc4ccccc45)cc3)c2)cc1 ; c1ccc(-c2nc3c(ccc4oc5ccc(-c6ccc(N(c7ccc(-c8cccc9c8oc8ccccc89)cc7)c7ccccc7-c7ccccc7)cc6)cc5c43)s2)cc1]

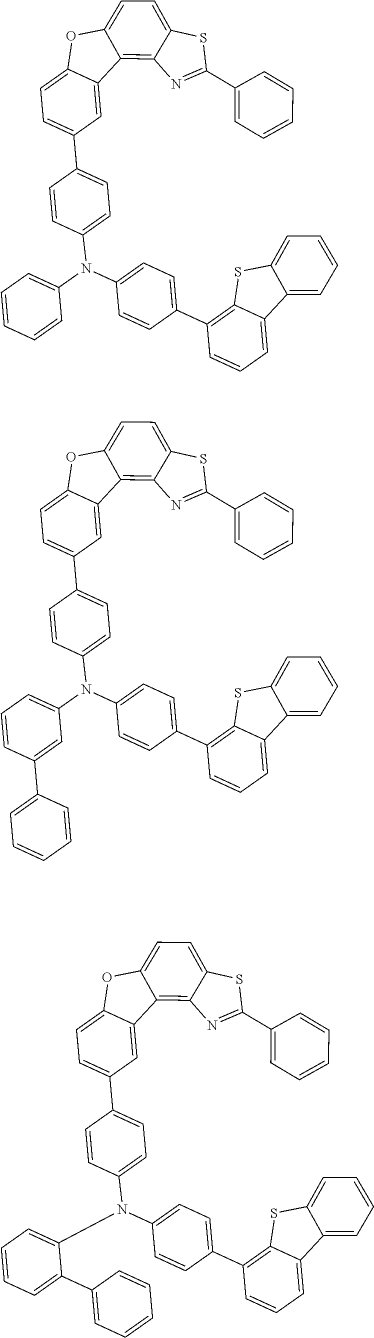 [SMILES: c1ccc(-c2nc3c(ccc4oc5ccc(-c6ccc(N(c7ccccc7)c7ccc(-c8cccc9c8sc8ccccc89)cc7)cc6)cc5c43)s2)cc1 ; c1ccc(-c2cccc(N(c3ccc(-c4ccc5oc6ccc7sc(-c8ccccc8)nc7c6c5c4)cc3)c3ccc(-c4cccc5c4sc4ccccc45)cc3)c2)cc1 ; c1ccc(-c2nc3c(ccc4oc5ccc(-c6ccc(N(c7ccc(-c8cccc9c8sc8ccccc89)cc7)c7ccccc7-c7ccccc7)cc6)cc5c43)s2)cc1]

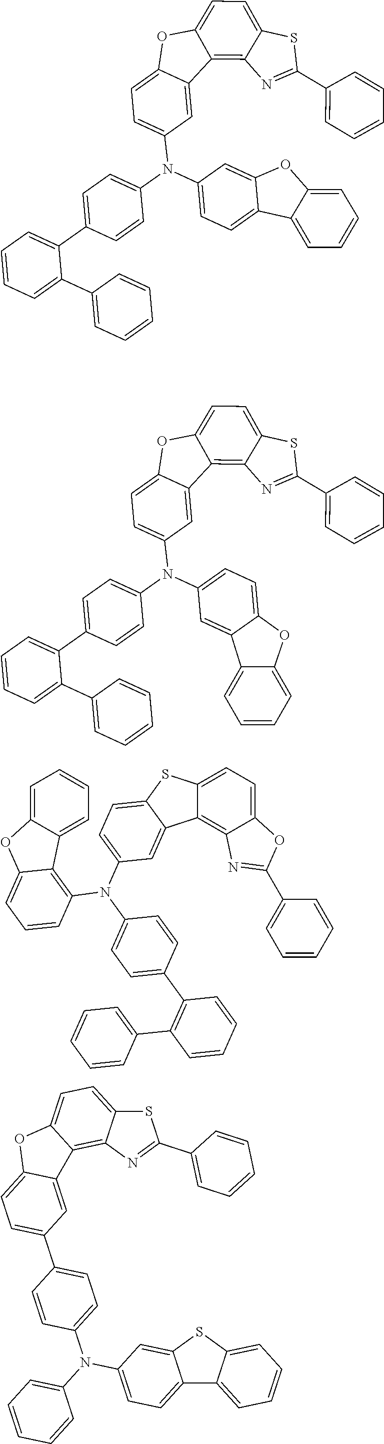

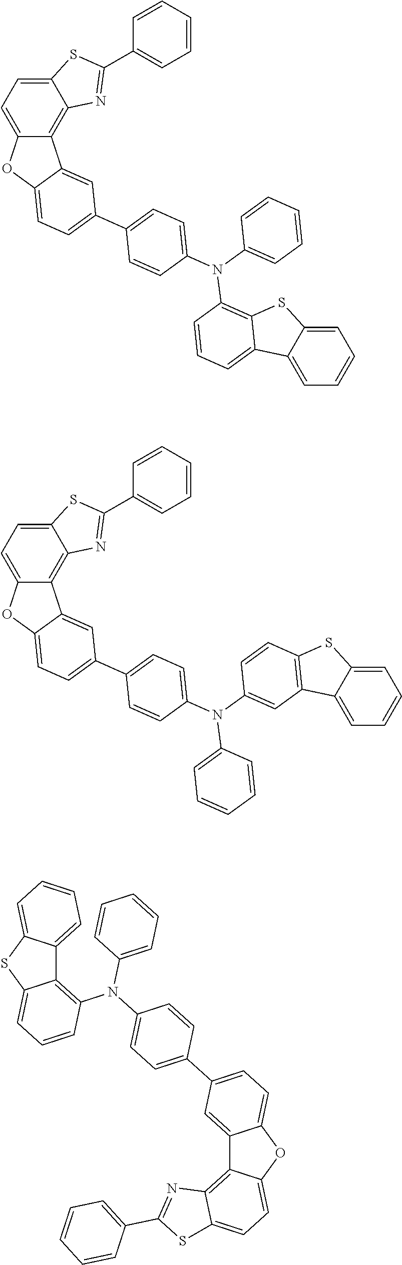 [SMILES: c1ccc(-c2nc3c(ccc4oc5ccc(-c6ccc(N(c7ccccc7)c7cccc8c7sc7ccccc78)cc6)cc5c43)s2)cc1 ; c1ccc(-c2nc3c(ccc4oc5ccc(-c6ccc(N(c7ccccc7)c7ccc8sc9ccccc9c8c7)cc6)cc5c43)s2)cc1 ; c1ccc(-c2nc3c(ccc4oc5ccc(-c6ccc(N(c7ccccc7)c7cccc8sc9ccccc9c78)cc6)cc5c43)s2)cc1]

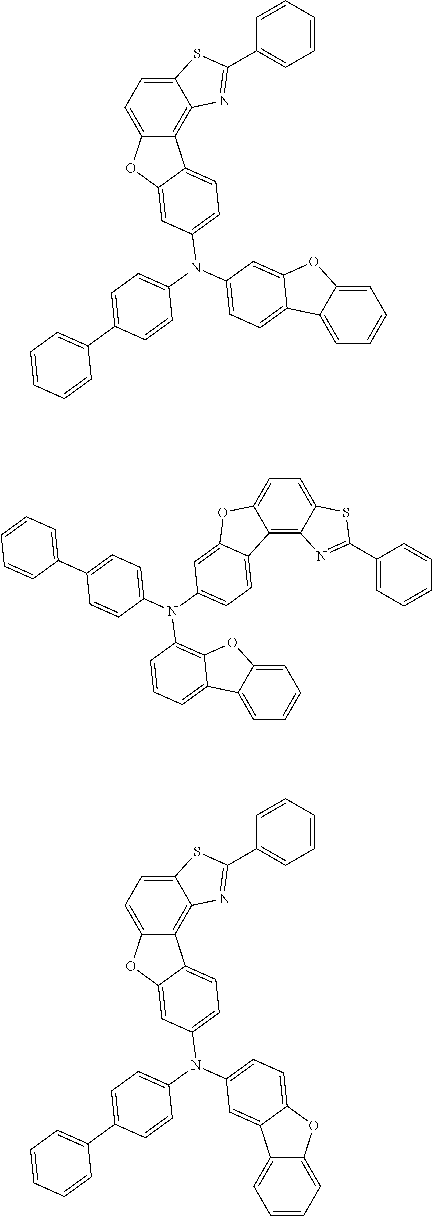 [SMILES: c1ccc(-c2ccc(N(c3ccc4c(c3)oc3ccccc34)c3ccc4c(c3)oc3ccc5sc(-c6ccccc6)nc5c34)cc2)cc1 ; c1ccc(-c2ccc(N(c3ccc4c(c3)oc3ccc5sc(-c6ccccc6)nc5c34)c3cccc4c3oc3ccccc34)cc2)cc1 ; c1ccc(-c2ccc(N(c3ccc4c(c3)oc3ccc5sc(-c6ccccc6)nc5c34)c3ccc4oc5ccccc5c4c3)cc2)cc1]

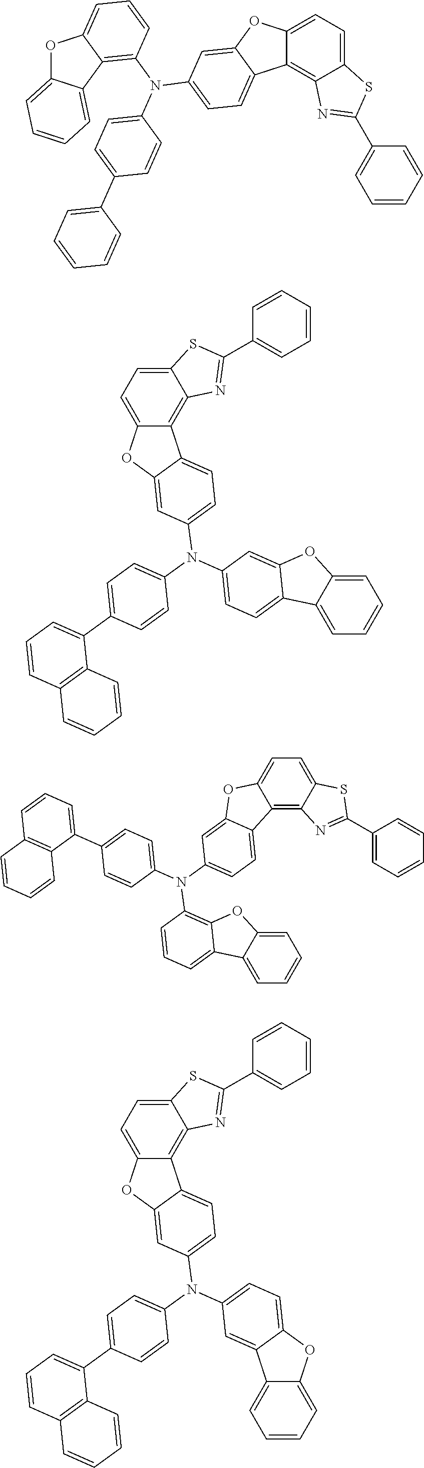

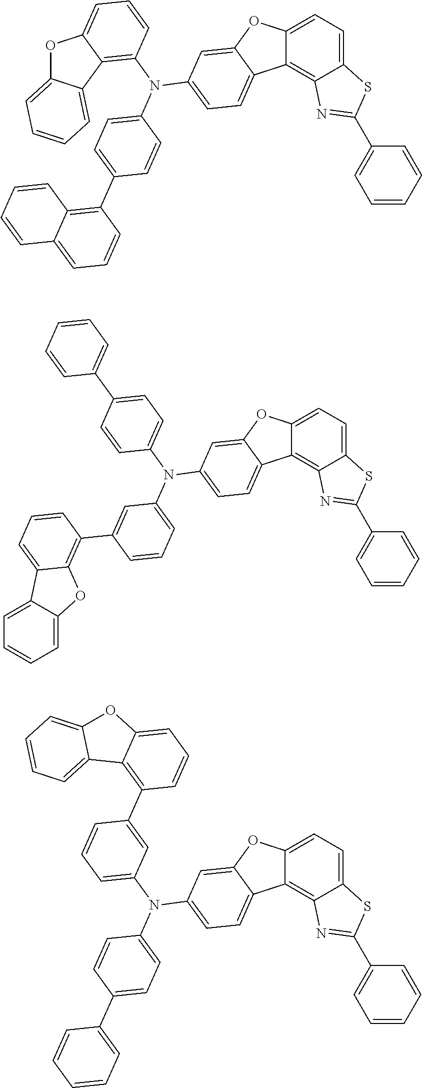 [SMILES: c1ccc(-c2nc3c(ccc4oc5cc(N(c6ccc(-c7cccc8ccccc78)cc6)c6cccc7oc8ccccc8c67)ccc5c43)s2)cc1 ; c1ccc(-c2ccc(N(c3cccc(-c4cccc5c4oc4ccccc45)c3)c3ccc4c(c3)oc3ccc5sc(-c6ccccc6)nc5c34)cc2)cc1 ; c1ccc(-c2ccc(N(c3cccc(-c4cccc5oc6ccccc6c45)c3)c3ccc4c(c3)oc3ccc5sc(-c6ccccc6)nc5c34)cc2)cc1]

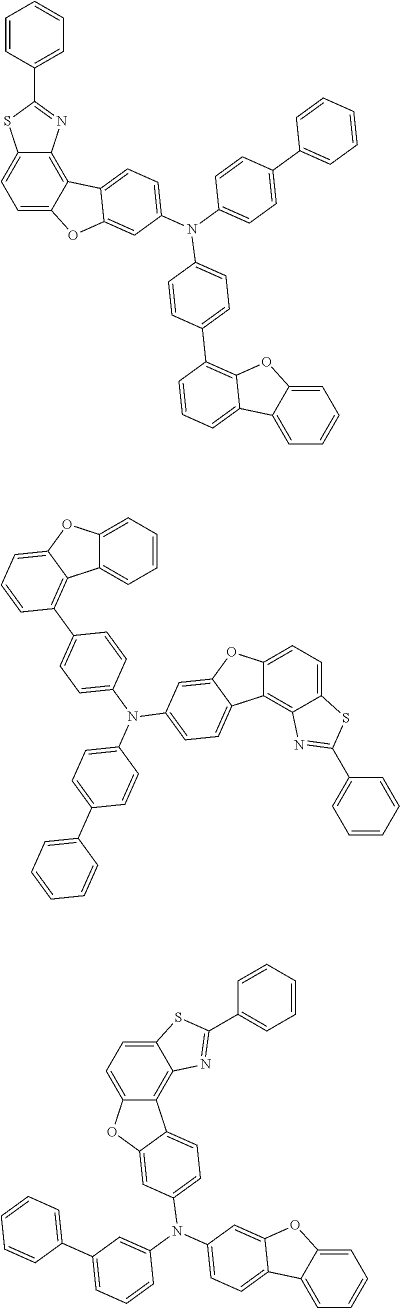 [SMILES: c1ccc(-c2ccc(N(c3ccc(-c4cccc5c4oc4ccccc45)cc3)c3ccc4c(c3)oc3ccc5sc(-c6ccccc6)nc5c34)cc2)cc1 ; c1ccc(-c2ccc(N(c3ccc(-c4cccc5oc6ccccc6c45)cc3)c3ccc4c(c3)oc3ccc5sc(-c6ccccc6)nc5c34)cc2)cc1 ; c1ccc(-c2cccc(N(c3ccc4c(c3)oc3ccccc34)c3ccc4c(c3)oc3ccc5sc(-c6ccccc6)nc5c34)c2)cc1]

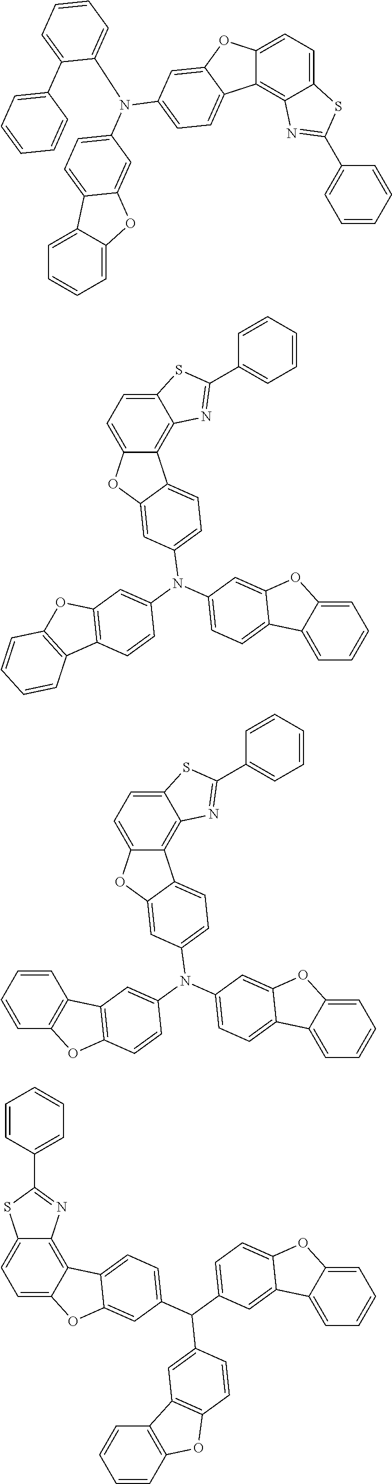 [SMILES: c1ccc(-c2nc3c(ccc4oc5cc(N(c6ccc7c(c6)oc6ccccc67)c6ccccc6-c6ccccc6)ccc5c43)s2)cc1 ; c1ccc(-c2nc3c(ccc4oc5cc(N(c6ccc7c(c6)oc6ccccc67)c6ccc7c(c6)oc6ccccc67)ccc5c43)s2)cc1 ; c1ccc(-c2nc3c(ccc4oc5cc(N(c6ccc7c(c6)oc6ccccc67)c6ccc7oc8ccccc8c7c6)ccc5c43)s2)cc1 ; c1ccc(-c2nc3c(ccc4oc5cc(C(c6ccc7oc8ccccc8c7c6)c6ccc7oc8ccccc8c7c6)ccc5c43)s2)cc1]

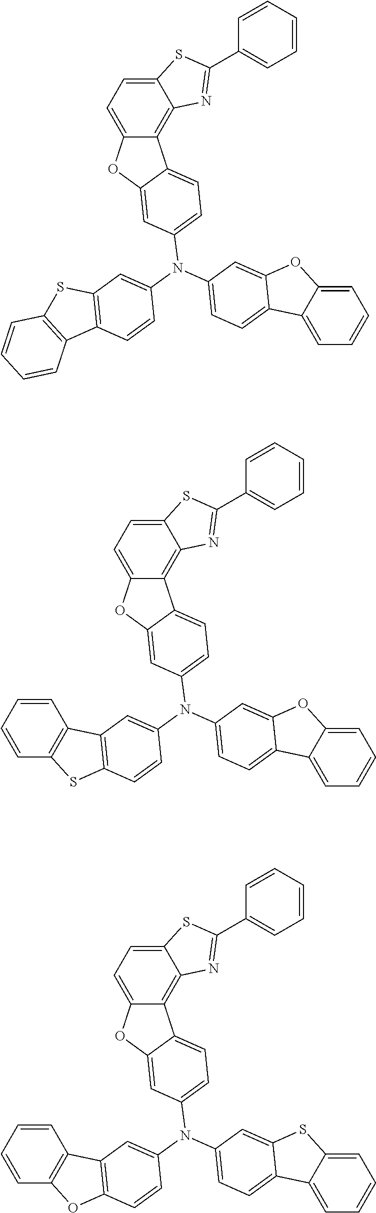 [SMILES: c1ccc(-c2nc3c(ccc4oc5cc(N(c6ccc7c(c6)oc6ccccc67)c6ccc7c(c6)sc6ccccc67)ccc5c43)s2)cc1 ; c1ccc(-c2nc3c(ccc4oc5cc(N(c6ccc7c(c6)oc6ccccc67)c6ccc7sc8ccccc8c7c6)ccc5c43)s2)cc1 ; c1ccc(-c2nc3c(ccc4oc5cc(N(c6ccc7c(c6)sc6ccccc67)c6ccc7oc8ccccc8c7c6)ccc5c43)s2)cc1]

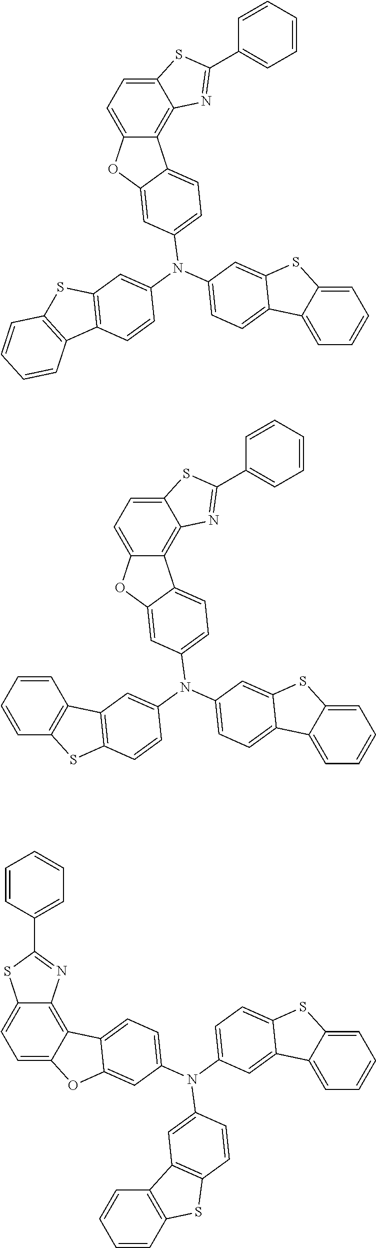 [SMILES: c1ccc(-c2nc3c(ccc4oc5cc(N(c6ccc7c(c6)sc6ccccc67)c6ccc7c(c6)sc6ccccc67)ccc5c43)s2)cc1 ; c1ccc(-c2nc3c(ccc4oc5cc(N(c6ccc7c(c6)sc6ccccc67)c6ccc7sc8ccccc8c7c6)ccc5c43)s2)cc1 ; c1ccc(-c2nc3c(ccc4oc5cc(N(c6ccc7sc8ccccc8c7c6)c6ccc7sc8ccccc8c7c6)ccc5c43)s2)cc1]

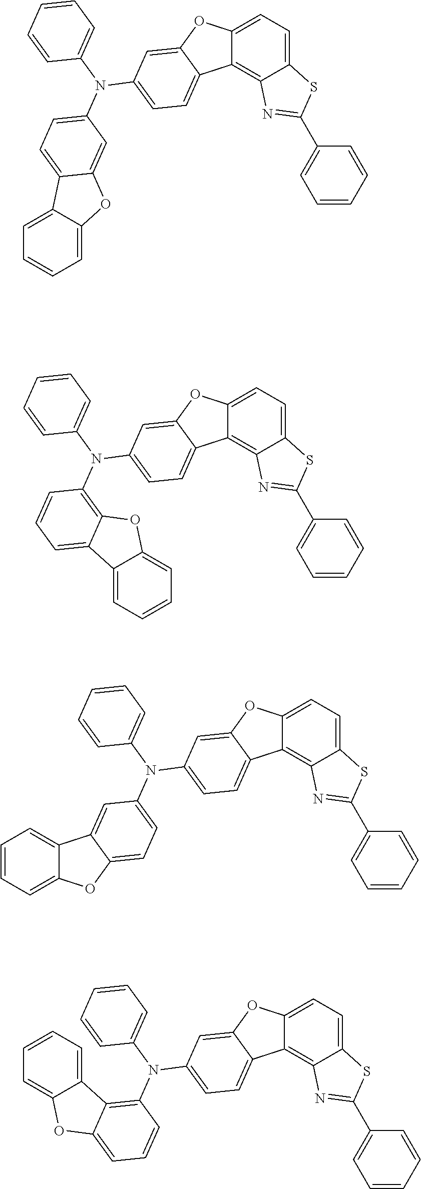 [SMILES: c1ccc(-c2nc3c(ccc4oc5cc(N(c6ccccc6)c6ccc7c(c6)oc6ccccc67)ccc5c43)s2)cc1 ; c1ccc(-c2nc3c(ccc4oc5cc(N(c6ccccc6)c6cccc7c6oc6ccccc67)ccc5c43)s2)cc1 ; c1ccc(-c2nc3c(ccc4oc5cc(N(c6ccccc6)c6ccc7oc8ccccc8c7c6)ccc5c43)s2)cc1 ; c1ccc(-c2nc3c(ccc4oc5cc(N(c6ccccc6)c6cccc7oc8ccccc8c67)ccc5c43)s2)cc1]

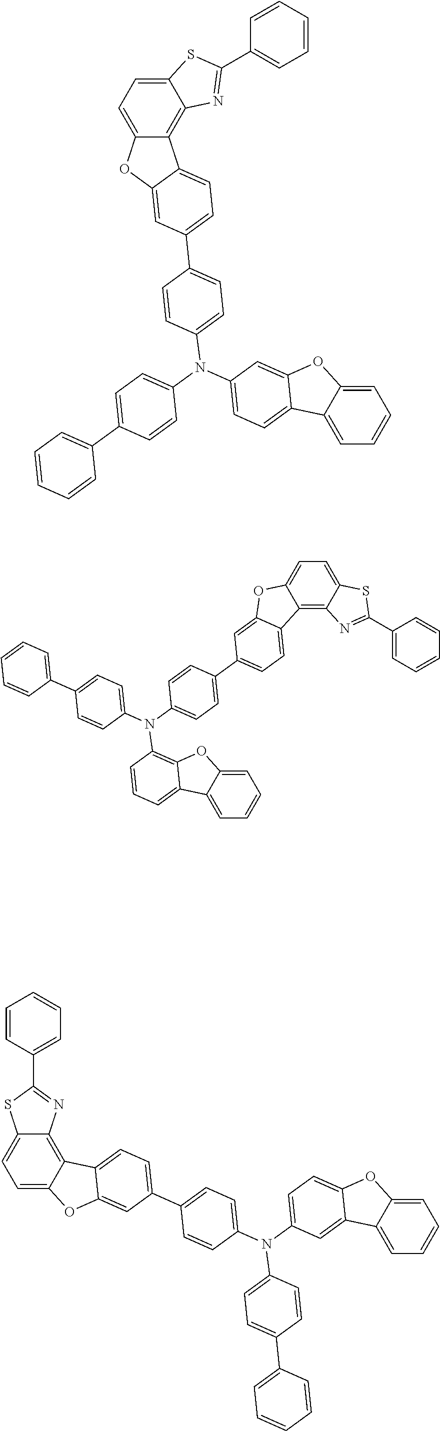 [SMILES: c1ccc(-c2ccc(N(c3ccc(-c4ccc5c(c4)oc4ccc6sc(-c7ccccc7)nc6c45)cc3)c3ccc4c(c3)oc3ccccc34)cc2)cc1 ; c1ccc(-c2ccc(N(c3ccc(-c4ccc5c(c4)oc4ccc6sc(-c7ccccc7)nc6c45)cc3)c3cccc4c3oc3ccccc34)cc2)cc1 ; c1ccc(-c2ccc(N(c3ccc(-c4ccc5c(c4)oc4ccc6sc(-c7ccccc7)nc6c45)cc3)c3ccc4oc5ccccc5c4c3)cc2)cc1]

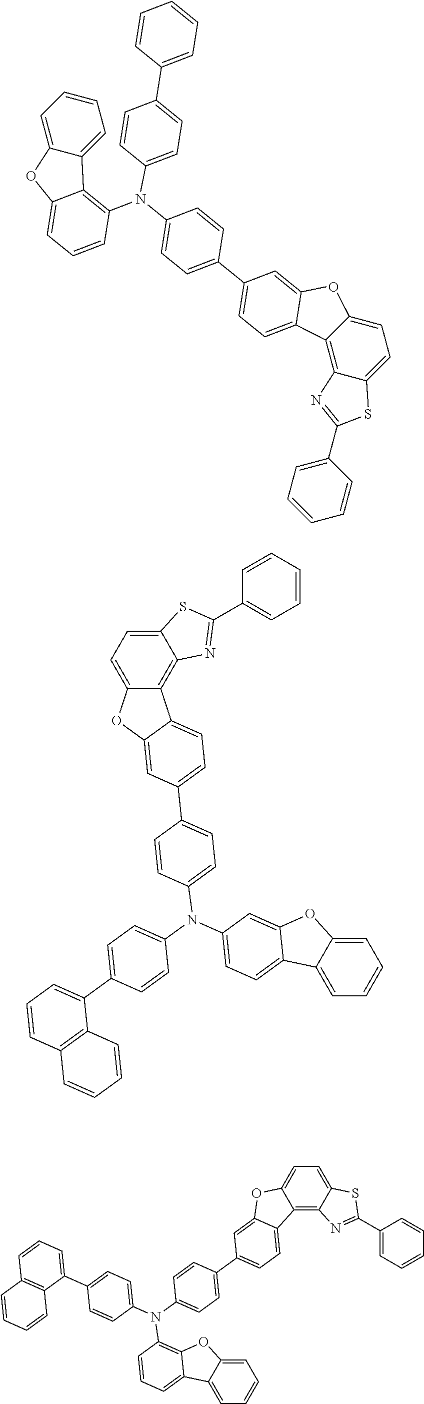 [SMILES: c1ccc(-c2ccc(N(c3ccc(-c4ccc5c(c4)oc4ccc6sc(-c7ccccc7)nc6c45)cc3)c3cccc4oc5ccccc5c34)cc2)cc1 ; c1ccc(-c2nc3c(ccc4oc5cc(-c6ccc(N(c7ccc(-c8cccc9ccccc89)cc7)c7ccc8c(c7)oc7ccccc78)cc6)ccc5c43)s2)cc1 ; c1ccc(-c2nc3c(ccc4oc5cc(-c6ccc(N(c7ccc(-c8cccc9ccccc89)cc7)c7cccc8c7oc7ccccc78)cc6)ccc5c43)s2)cc1]

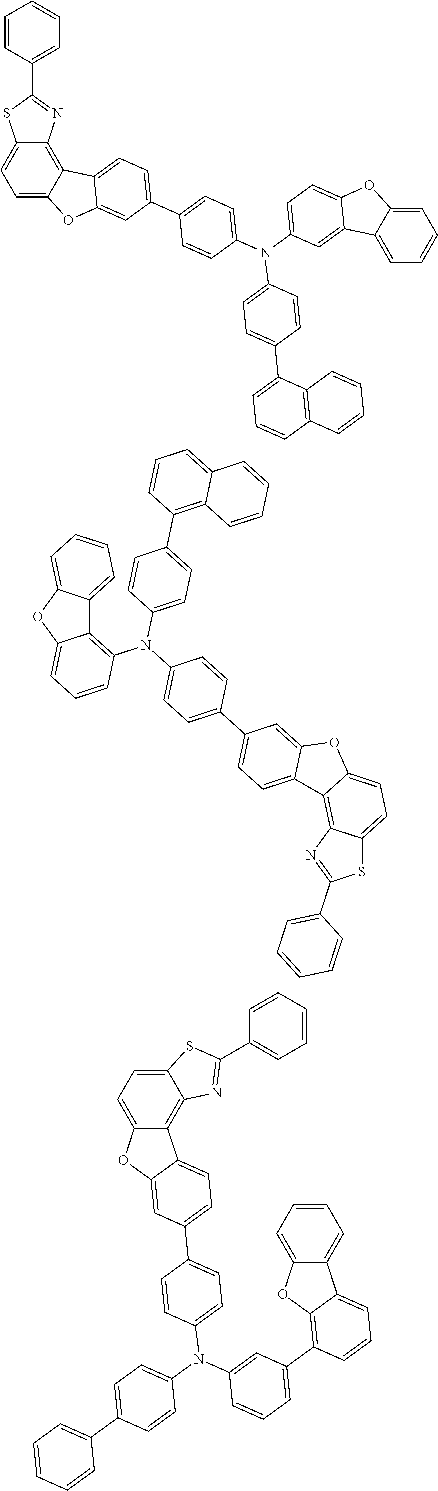

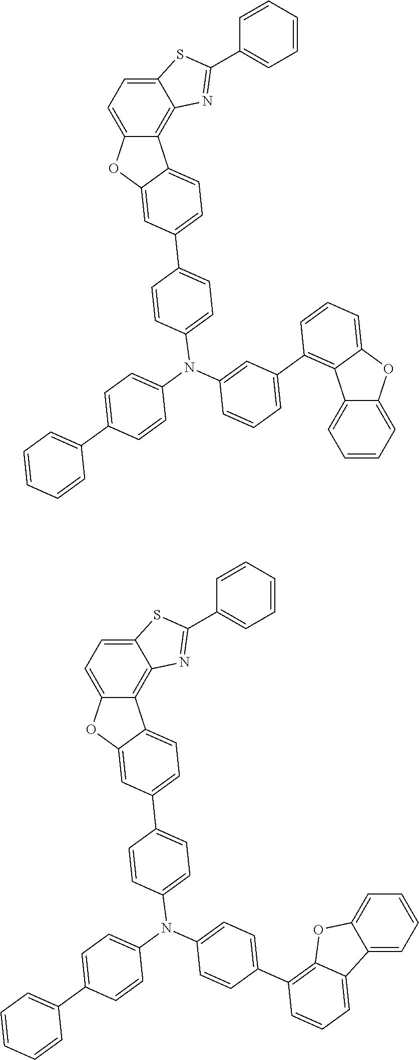 [SMILES: c1ccc(-c2ccc(N(c3ccc(-c4ccc5c(c4)oc4ccc6sc(-c7ccccc7)nc6c45)cc3)c3cccc(-c4cccc5oc6ccccc6c45)c3)cc2)cc1 ; c1ccc(-c2ccc(N(c3ccc(-c4ccc5c(c4)oc4ccc6sc(-c7ccccc7)nc6c45)cc3)c3ccc(-c4cccc5c4oc4ccccc45)cc3)cc2)cc1]

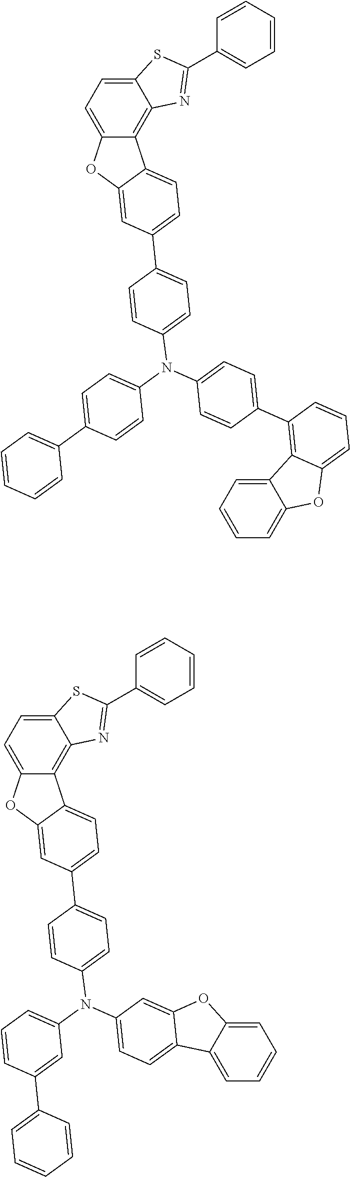 [SMILES: c1ccc(-c2ccc(N(c3ccc(-c4ccc5c(c4)oc4ccc6sc(-c7ccccc7)nc6c45)cc3)c3ccc(-c4cccc5oc6ccccc6c45)cc3)cc2)cc1 ; c1ccc(-c2cccc(N(c3ccc(-c4ccc5c(c4)oc4ccc6sc(-c7ccccc7)nc6c45)cc3)c3ccc4c(c3)oc3ccccc34)c2)cc1]

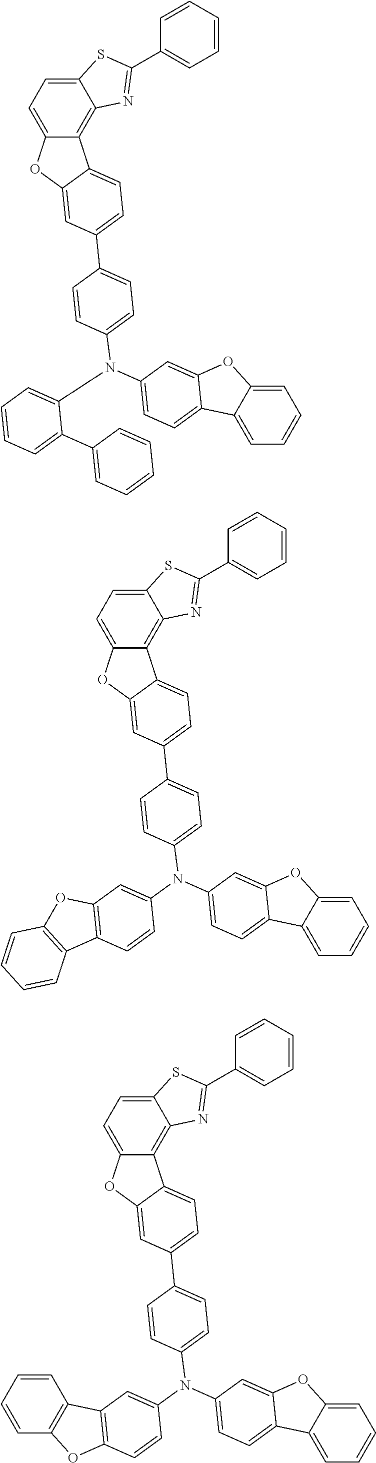 [SMILES: c1ccc(-c2nc3c(ccc4oc5cc(-c6ccc(N(c7ccc8c(c7)oc7ccccc78)c7ccccc7-c7ccccc7)cc6)ccc5c43)s2)cc1 ; c1ccc(-c2nc3c(ccc4oc5cc(-c6ccc(N(c7ccc8c(c7)oc7ccccc78)c7ccc8c(c7)oc7ccccc78)cc6)ccc5c43)s2)cc1 ; c1ccc(-c2nc3c(ccc4oc5cc(-c6ccc(N(c7ccc8c(c7)oc7ccccc78)c7ccc8oc9ccccc9c8c7)cc6)ccc5c43)s2)cc1]

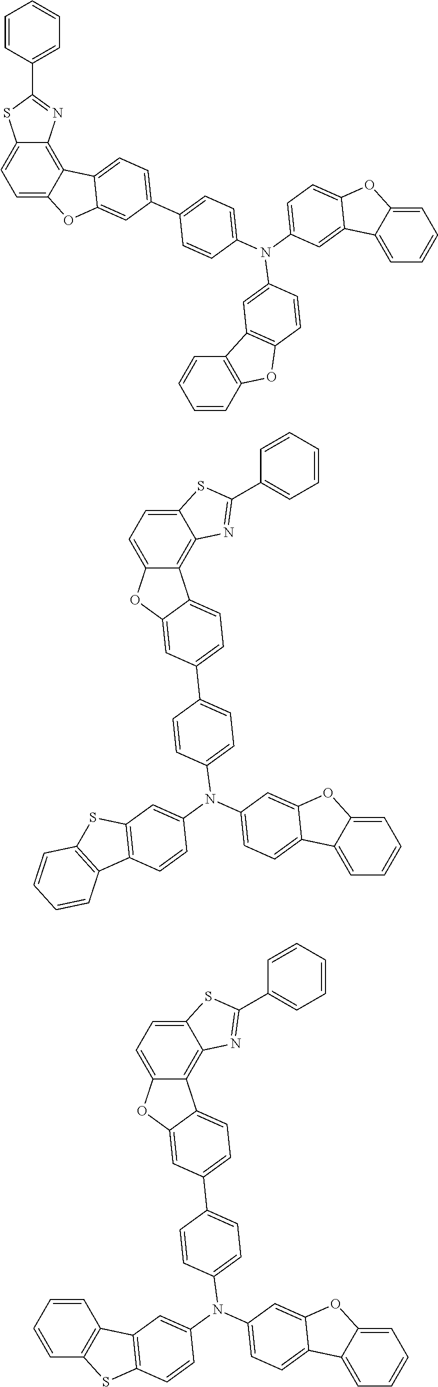 [SMILES: c1ccc(-c2nc3c(ccc4oc5cc(-c6ccc(N(c7ccc8oc9ccccc9c8c7)c7ccc8oc9ccccc9c8c7)cc6)ccc5c43)s2)cc1 ; c1ccc(-c2nc3c(ccc4oc5cc(-c6ccc(N(c7ccc8c(c7)oc7ccccc78)c7ccc8c(c7)sc7ccccc78)cc6)ccc5c43)s2)cc1 ; c1ccc(-c2nc3c(ccc4oc5cc(-c6ccc(N(c7ccc8c(c7)oc7ccccc78)c7ccc8sc9ccccc9c8c7)cc6)ccc5c43)s2)cc1]

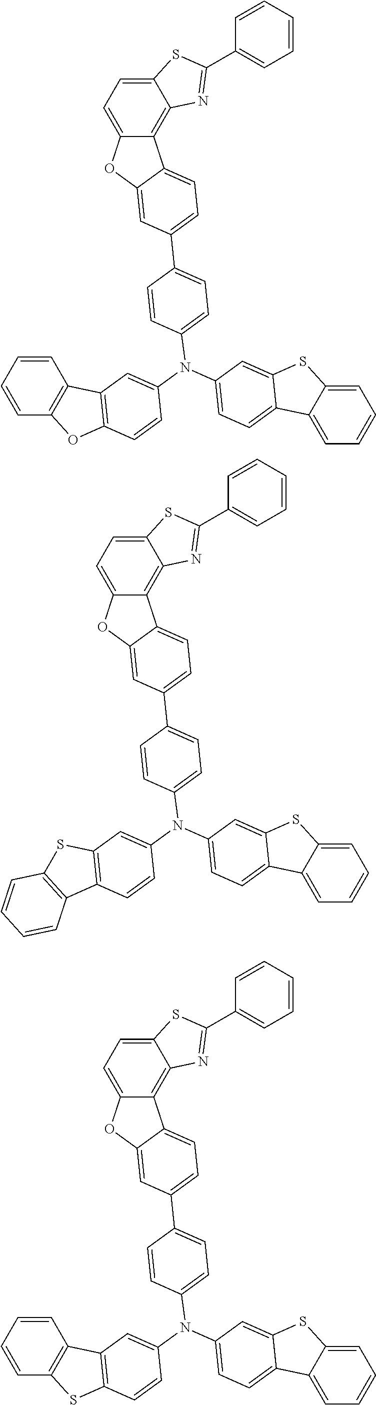

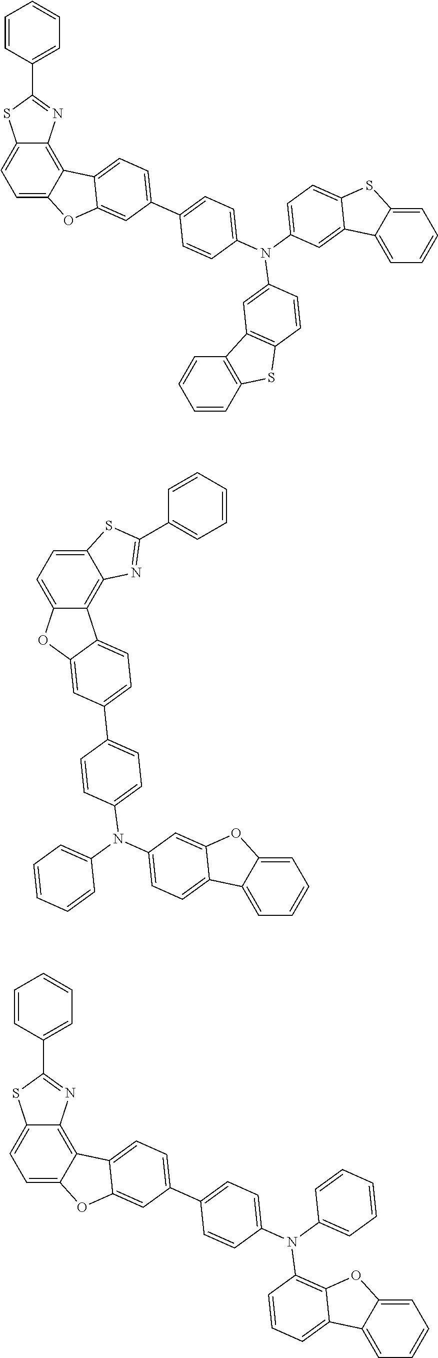 [SMILES: c1ccc(-c2nc3c(ccc4oc5cc(-c6ccc(N(c7ccc8sc9ccccc9c8c7)c7ccc8sc9ccccc9c8c7)cc6)ccc5c43)s2)cc1 ; c1ccc(-c2nc3c(ccc4oc5cc(-c6ccc(N(c7ccccc7)c7ccc8c(c7)oc7ccccc78)cc6)ccc5c43)s2)cc1 ; c1ccc(-c2nc3c(ccc4oc5cc(-c6ccc(N(c7ccccc7)c7cccc8c7oc7ccccc78)cc6)ccc5c43)s2)cc1]

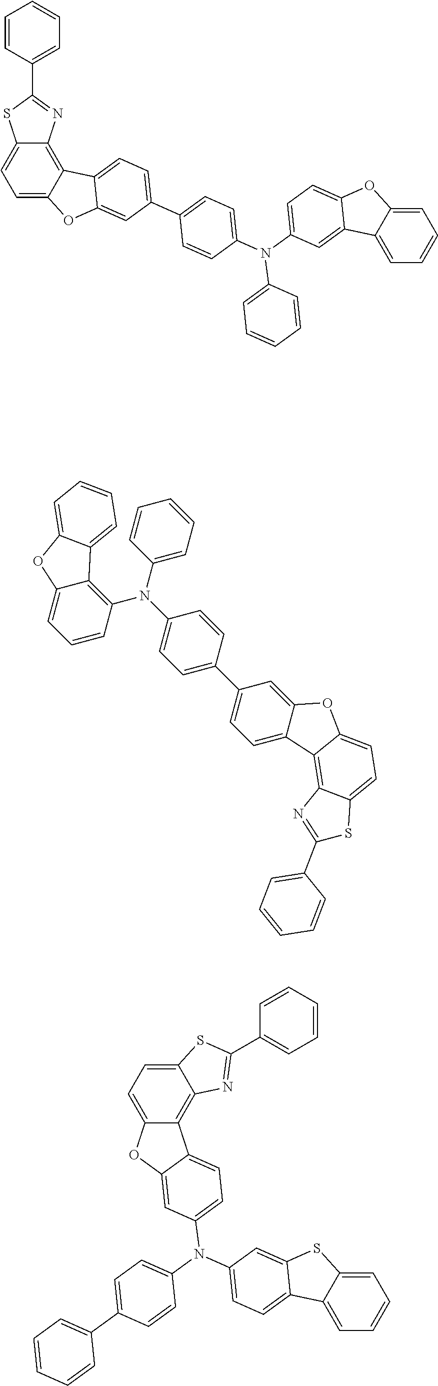 [SMILES: c1ccc(-c2nc3c(ccc4oc5cc(-c6ccc(N(c7ccccc7)c7ccc8oc9ccccc9c8c7)cc6)ccc5c43)s2)cc1 ; c1ccc(-c2nc3c(ccc4oc5cc(-c6ccc(N(c7ccccc7)c7cccc8oc9ccccc9c78)cc6)ccc5c43)s2)cc1 ; c1ccc(-c2ccc(N(c3ccc4c(c3)oc3ccc5sc(-c6ccccc6)nc5c34)c3ccc4c(c3)sc3ccccc34)cc2)cc1]

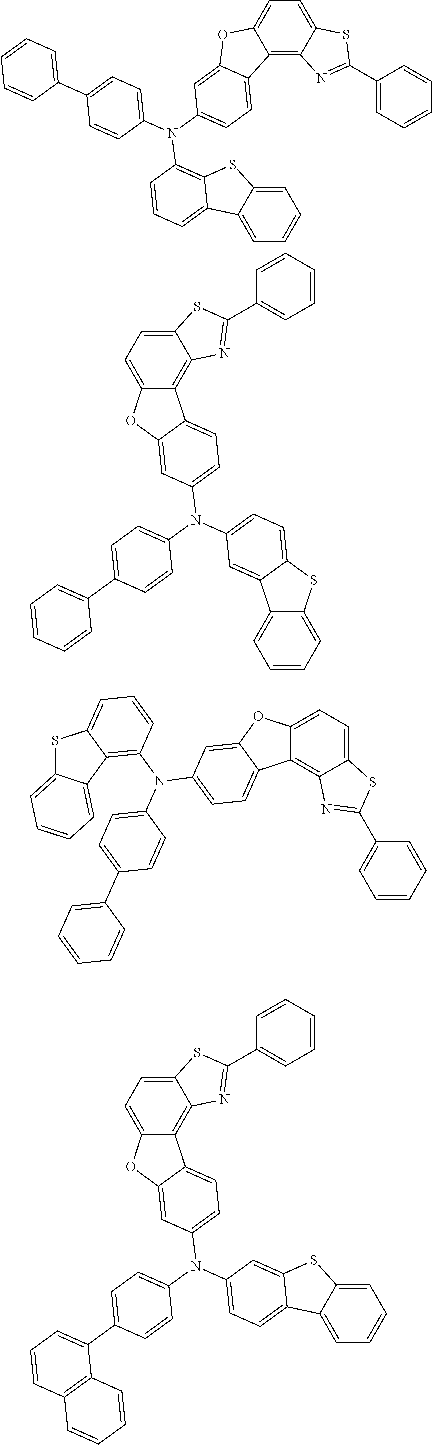

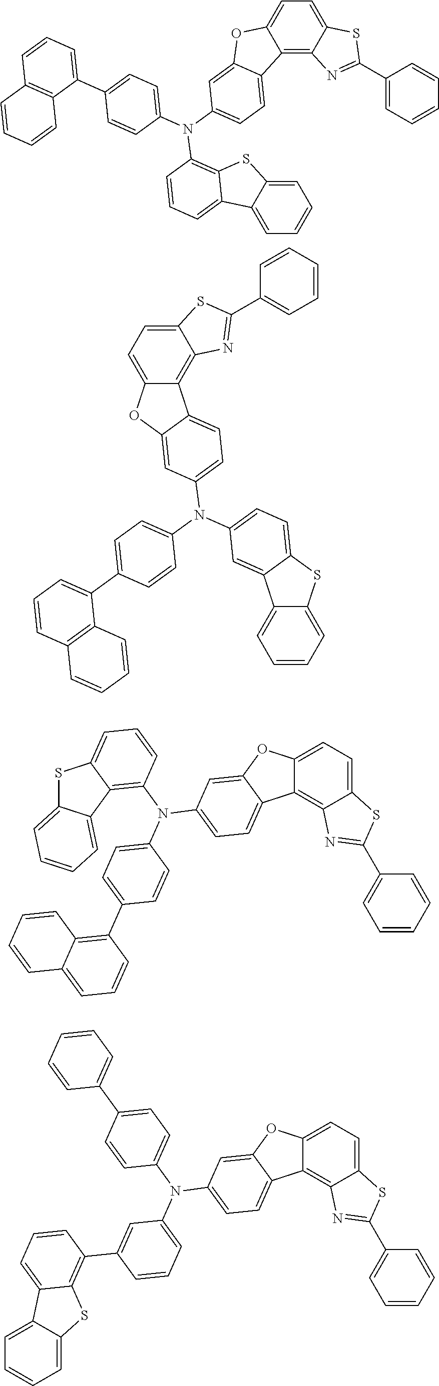

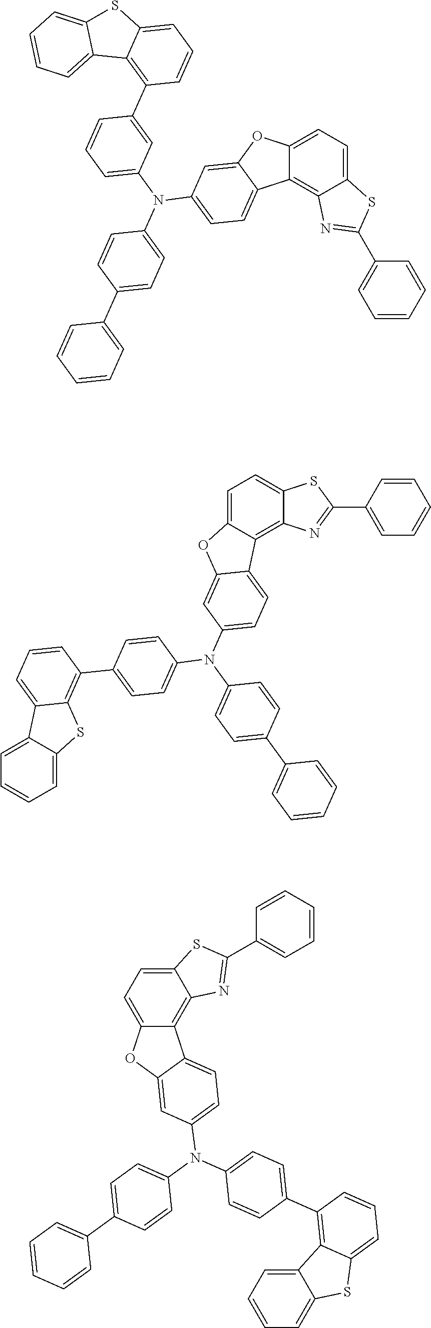 [SMILES: c1ccc(-c2ccc(N(c3cccc(-c4cccc5sc6ccccc6c45)c3)c3ccc4c(c3)oc3ccc5sc(-c6ccccc6)nc5c34)cc2)cc1 ; c1ccc(-c2ccc(N(c3ccc(-c4cccc5c4sc4ccccc45)cc3)c3ccc4c(c3)oc3ccc5sc(-c6ccccc6)nc5c34)cc2)cc1 ; c1ccc(-c2ccc(N(c3ccc(-c4cccc5sc6ccccc6c45)cc3)c3ccc4c(c3)oc3ccc5sc(-c6ccccc6)nc5c34)cc2)cc1]

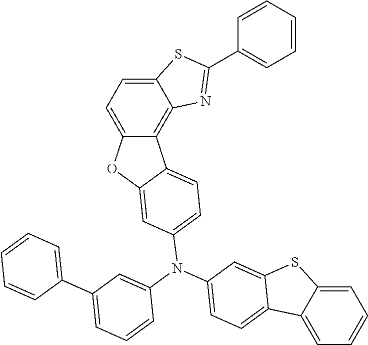 [SMILES: c1ccc(-c2cccc(N(c3ccc4c(c3)oc3ccc5sc(-c6ccccc6)nc5c34)c3ccc4c(c3)sc3ccccc34)c2)cc1]

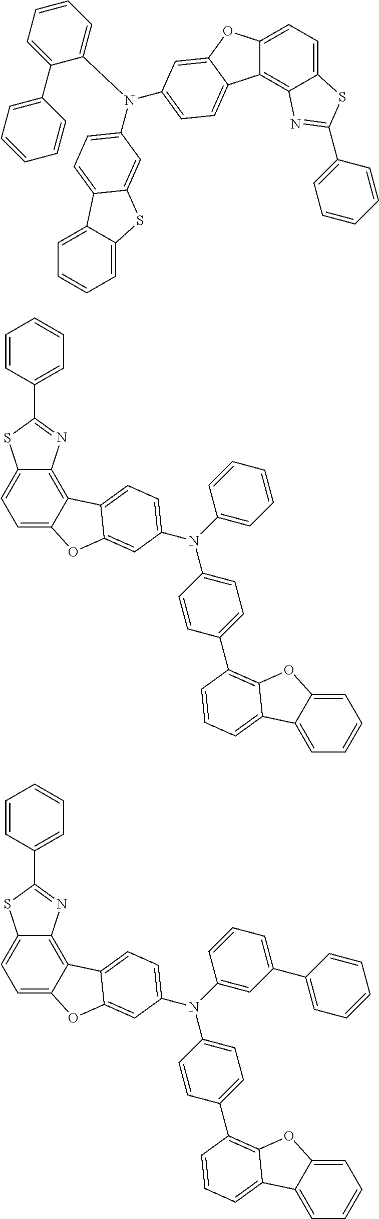 [SMILES: c1ccc(-c2nc3c(ccc4oc5cc(N(c6ccc7c(c6)sc6ccccc67)c6ccccc6-c6ccccc6)ccc5c43)s2)cc1 ; c1ccc(-c2nc3c(ccc4oc5cc(N(c6ccccc6)c6ccc(-c7cccc8c7oc7ccccc78)cc6)ccc5c43)s2)cc1 ; c1ccc(-c2cccc(N(c3ccc(-c4cccc5c4oc4ccccc45)cc3)c3ccc4c(c3)oc3ccc5sc(-c6ccccc6)nc5c34)c2)cc1]

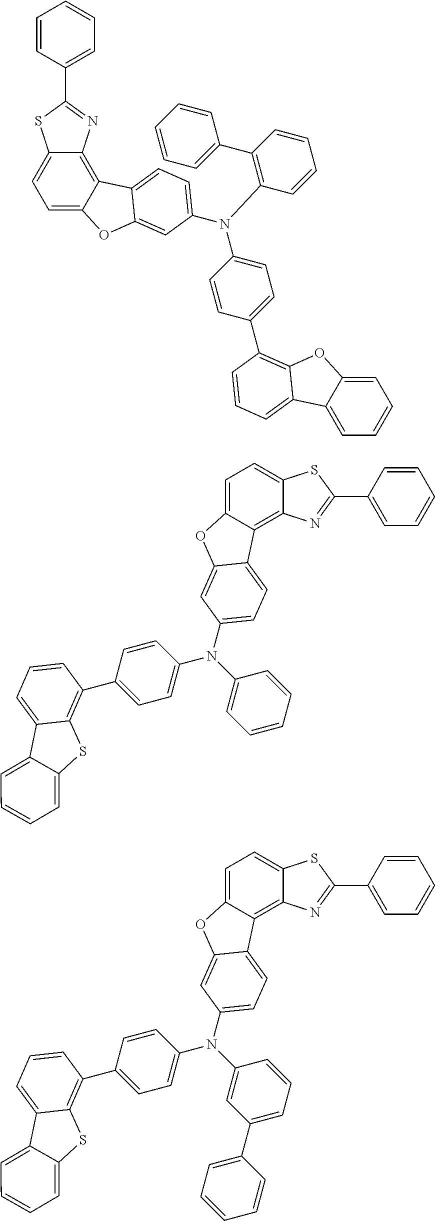

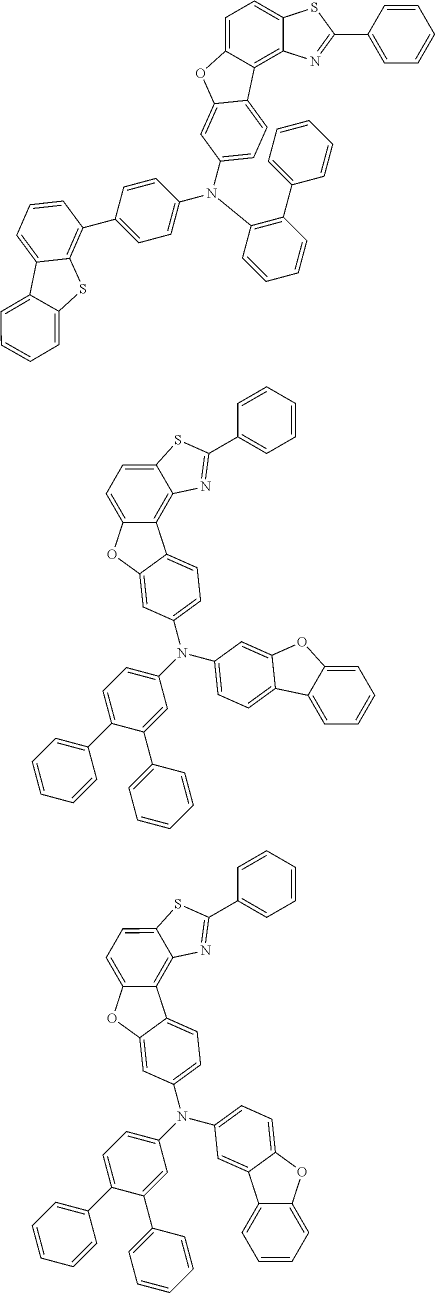 [SMILES: c1ccc(-c2nc3c(ccc4oc5cc(N(c6ccc(-c7cccc8c7sc7ccccc78)cc6)c6ccccc6-c6ccccc6)ccc5c43)s2)cc1 ; c1ccc(-c2nc3c(ccc4oc5cc(N(c6ccc(-c7ccccc7)c(-c7ccccc7)c6)c6ccc7c(c6)oc6ccccc67)ccc5c43)s2)cc1 ; c1ccc(-c2nc3c(ccc4oc5cc(N(c6ccc(-c7ccccc7)c(-c7ccccc7)c6)c6ccc7oc8ccccc8c7c6)ccc5c43)s2)cc1]

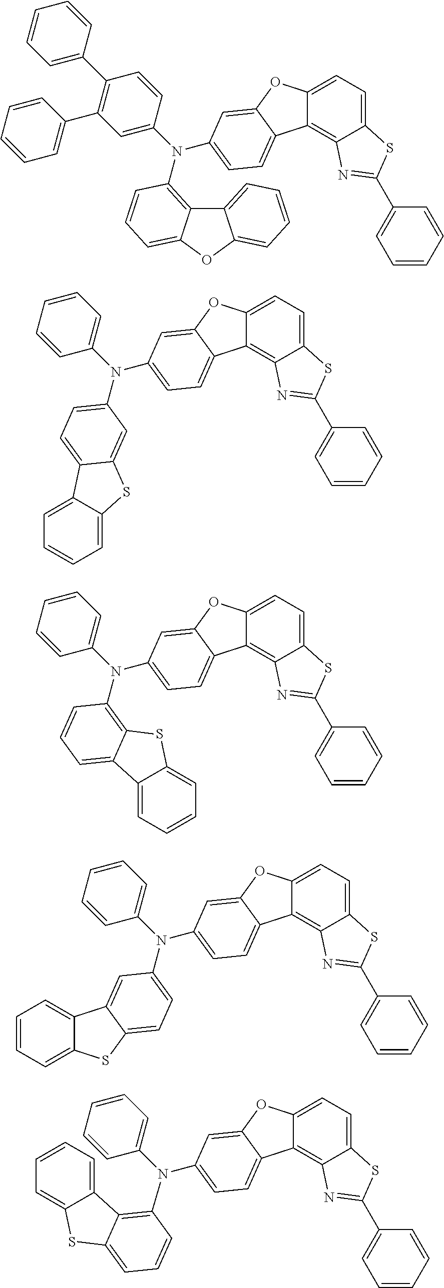

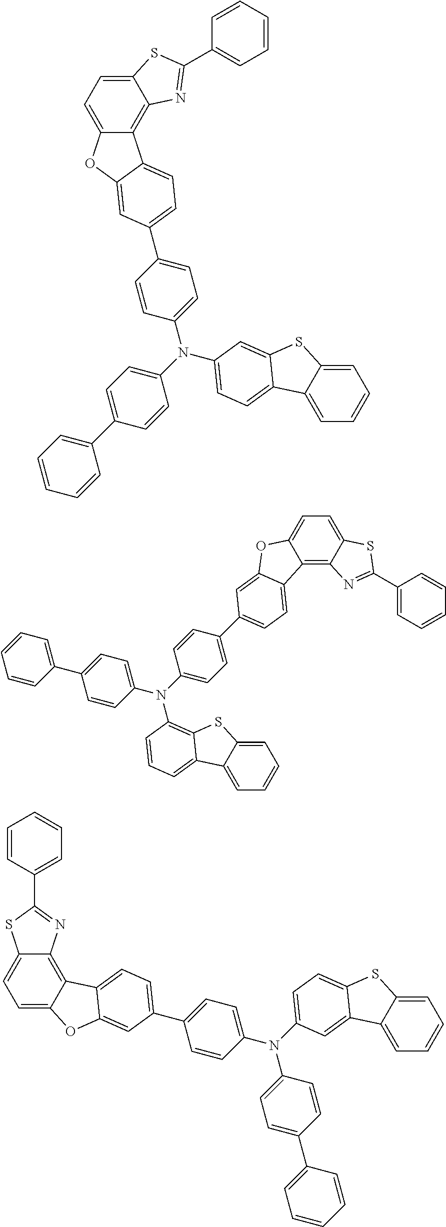 [SMILES: c1ccc(-c2ccc(N(c3ccc(-c4ccc5c(c4)oc4ccc6sc(-c7ccccc7)nc6c45)cc3)c3ccc4c(c3)sc3ccccc34)cc2)cc1 ; c1ccc(-c2ccc(N(c3ccc(-c4ccc5c(c4)oc4ccc6sc(-c7ccccc7)nc6c45)cc3)c3cccc4c3sc3ccccc34)cc2)cc1 ; c1ccc(-c2ccc(N(c3ccc(-c4ccc5c(c4)oc4ccc6sc(-c7ccccc7)nc6c45)cc3)c3ccc4sc5ccccc5c4c3)cc2)cc1]

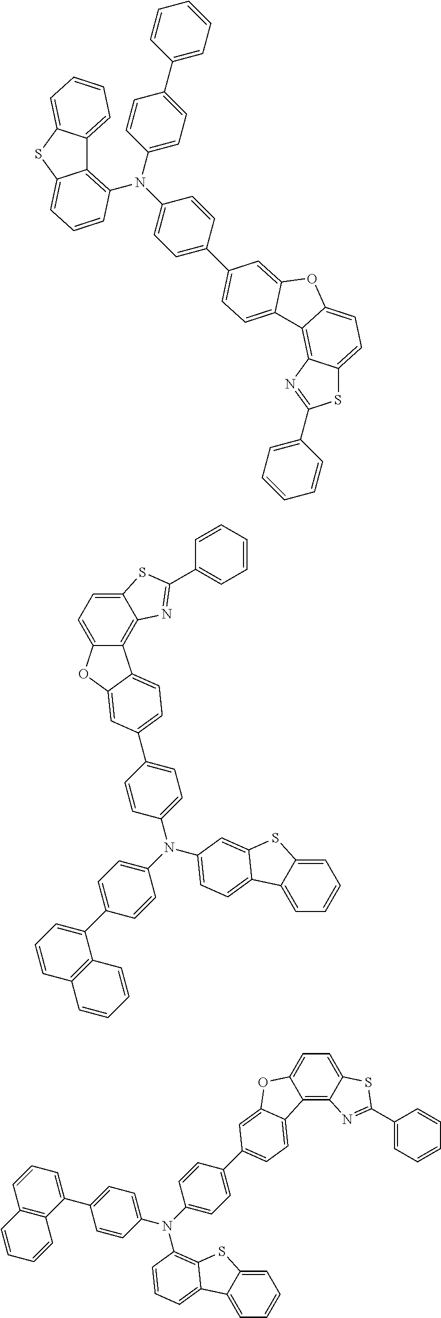 [SMILES: c1ccc(-c2ccc(N(c3ccc(-c4ccc5c(c4)oc4ccc6sc(-c7ccccc7)nc6c45)cc3)c3cccc4sc5ccccc5c34)cc2)cc1 ; c1ccc(-c2nc3c(ccc4oc5cc(-c6ccc(N(c7ccc(-c8cccc9ccccc89)cc7)c7ccc8c(c7)sc7ccccc78)cc6)ccc5c43)s2)cc1 ; c1ccc(-c2nc3c(ccc4oc5cc(-c6ccc(N(c7ccc(-c8cccc9ccccc89)cc7)c7cccc8c7sc7ccccc78)cc6)ccc5c43)s2)cc1]

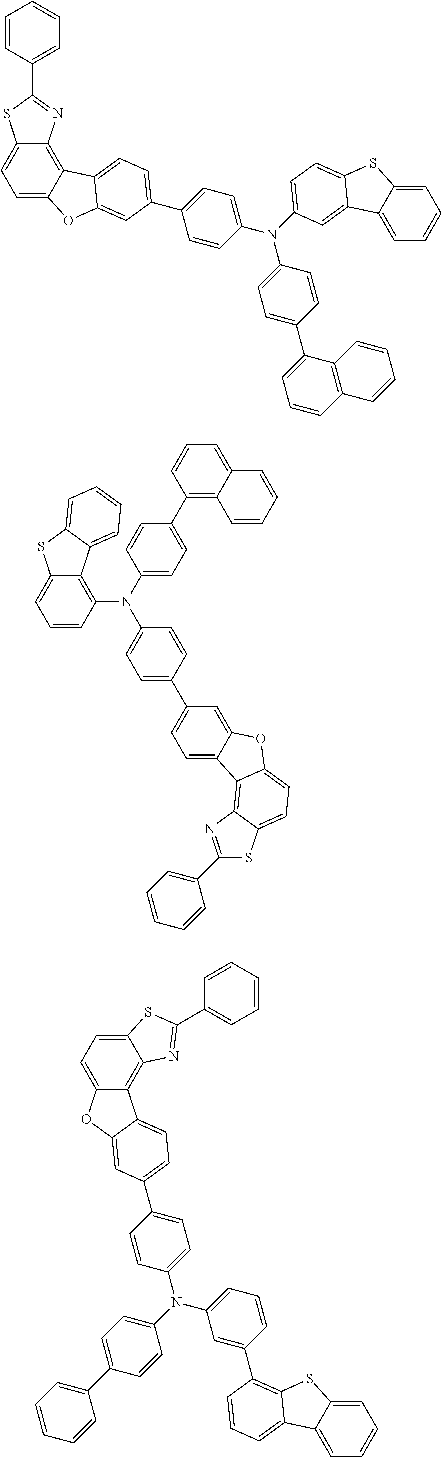 [SMILES: c1ccc(-c2nc3c(ccc4oc5cc(-c6ccc(N(c7ccc(-c8cccc9ccccc89)cc7)c7ccc8sc9ccccc9c8c7)cc6)ccc5c43)s2)cc1 ; c1ccc(-c2nc3c(ccc4oc5cc(-c6ccc(N(c7ccc(-c8cccc9ccccc89)cc7)c7cccc8sc9ccccc9c78)cc6)ccc5c43)s2)cc1 ; c1ccc(-c2ccc(N(c3ccc(-c4ccc5c(c4)oc4ccc6sc(-c7ccccc7)nc6c45)cc3)c3cccc(-c4cccc5c4sc4ccccc45)c3)cc2)cc1]

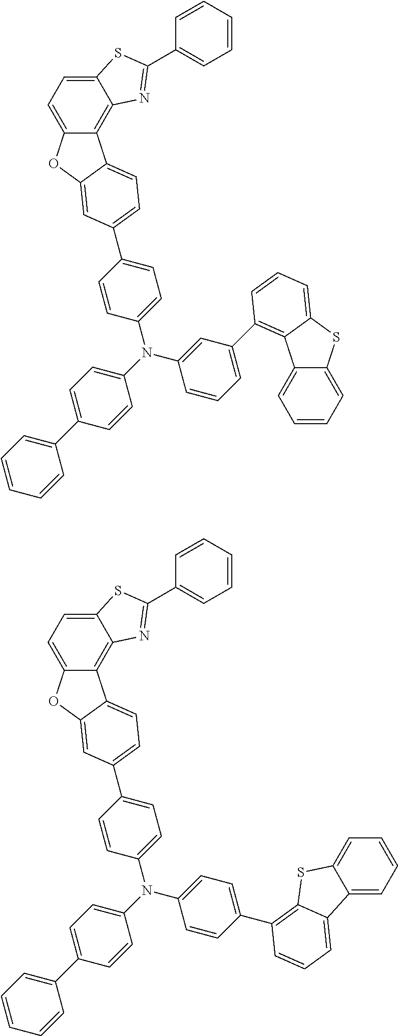 [SMILES: c1ccc(-c2ccc(N(c3ccc(-c4ccc5c(c4)oc4ccc6sc(-c7ccccc7)nc6c45)cc3)c3cccc(-c4cccc5sc6ccccc6c45)c3)cc2)cc1 ; c1ccc(-c2ccc(N(c3ccc(-c4ccc5c(c4)oc4ccc6sc(-c7ccccc7)nc6c45)cc3)c3ccc(-c4cccc5c4sc4ccccc45)cc3)cc2)cc1]

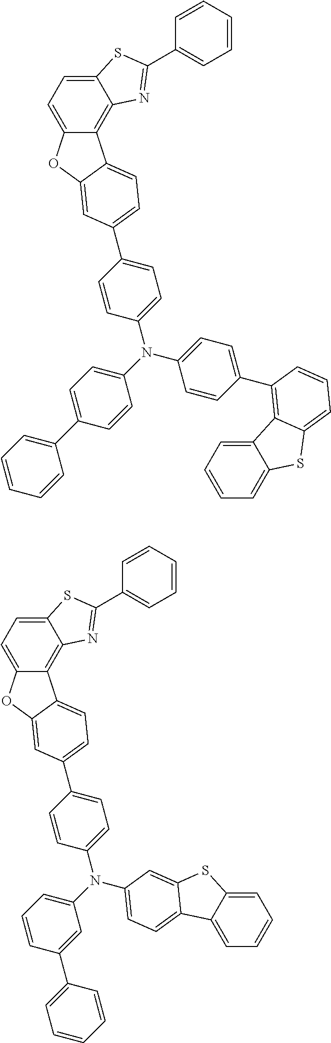 [SMILES: c1ccc(-c2ccc(N(c3ccc(-c4ccc5c(c4)oc4ccc6sc(-c7ccccc7)nc6c45)cc3)c3ccc(-c4cccc5sc6ccccc6c45)cc3)cc2)cc1 ; c1ccc(-c2cccc(N(c3ccc(-c4ccc5c(c4)oc4ccc6sc(-c7ccccc7)nc6c45)cc3)c3ccc4c(c3)sc3ccccc34)c2)cc1]

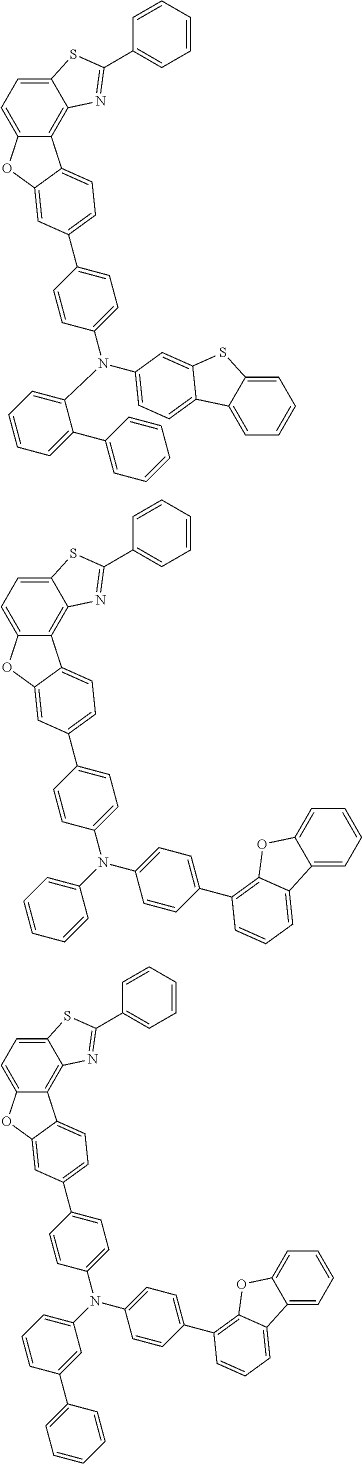

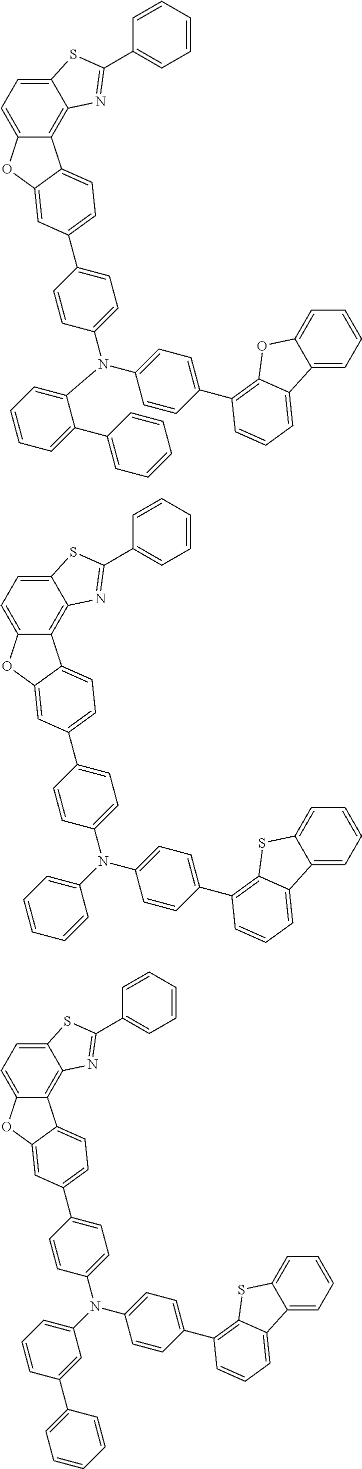

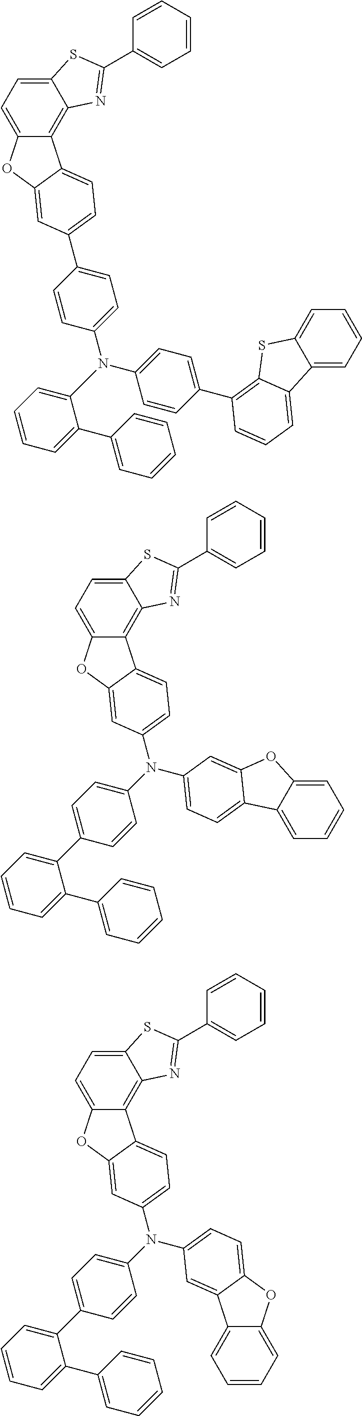 [SMILES: c1ccc(-c2nc3c(ccc4oc5cc(-c6ccc(N(c7ccc(-c8cccc9c8sc8ccccc89)cc7)c7ccccc7-c7ccccc7)cc6)ccc5c43)s2)cc1 ; c1ccc(-c2nc3c(ccc4oc5cc(N(c6ccc(-c7ccccc7-c7ccccc7)cc6)c6ccc7c(c6)oc6ccccc67)ccc5c43)s2)cc1 ; c1ccc(-c2nc3c(ccc4oc5cc(N(c6ccc(-c7ccccc7-c7ccccc7)cc6)c6ccc7oc8ccccc8c7c6)ccc5c43)s2)cc1]

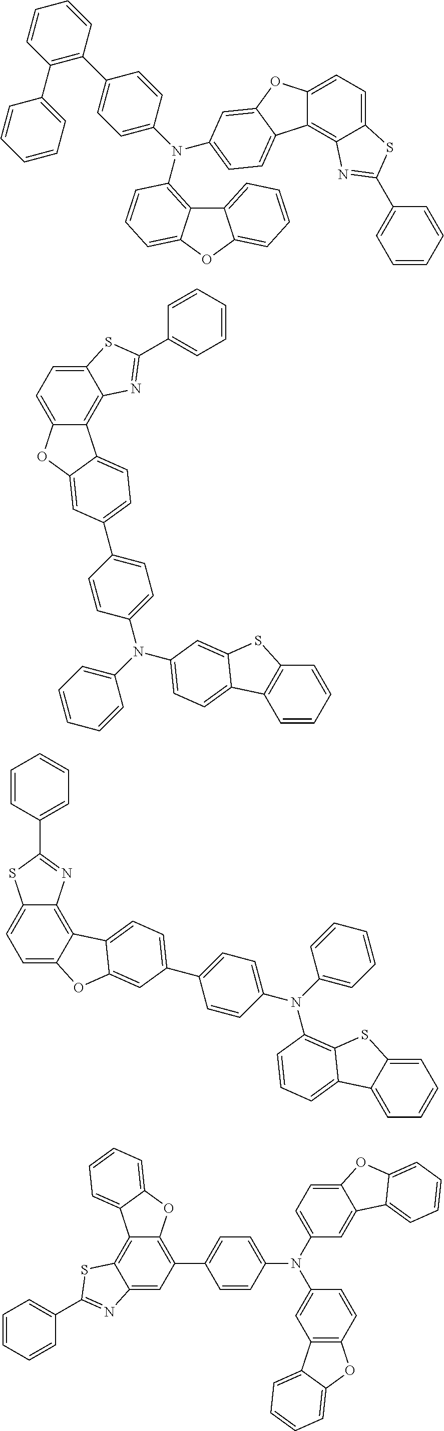

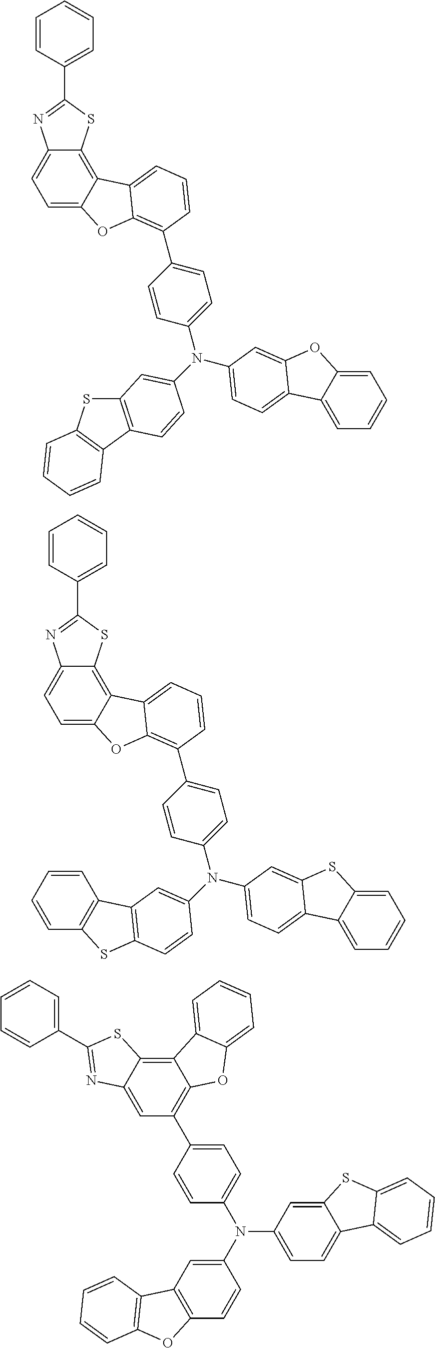

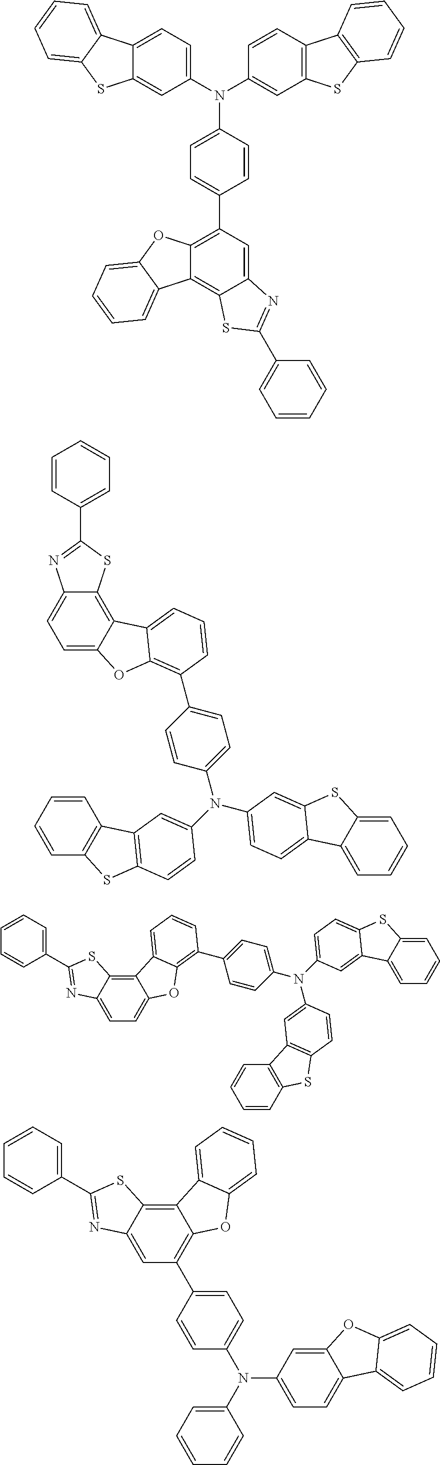

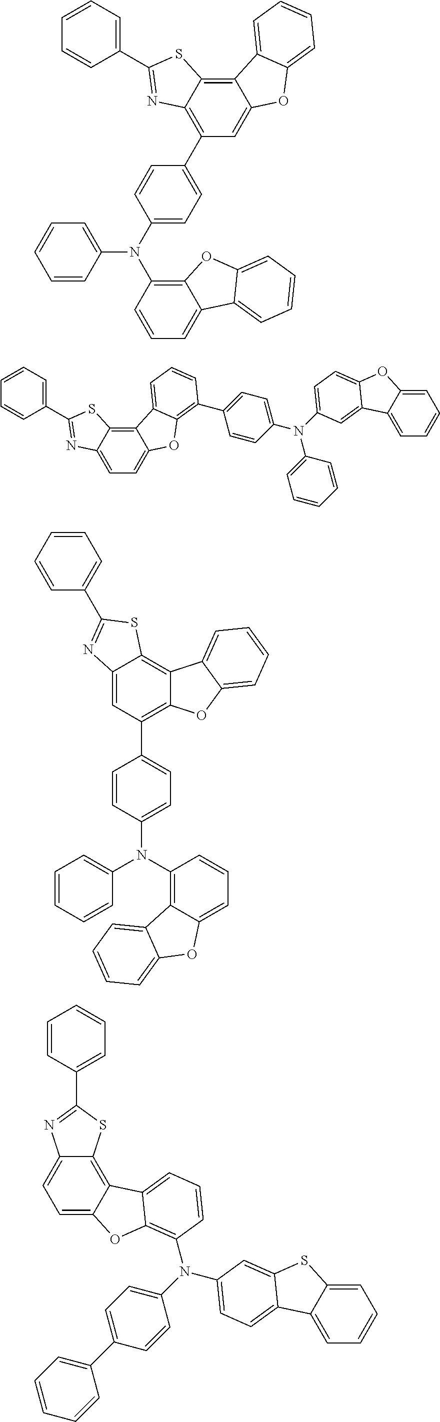

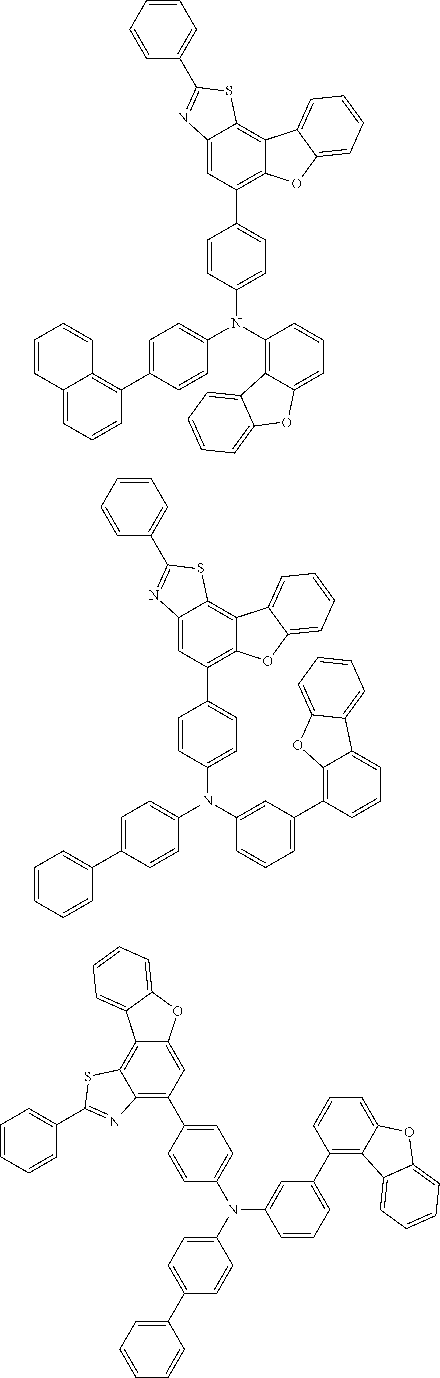

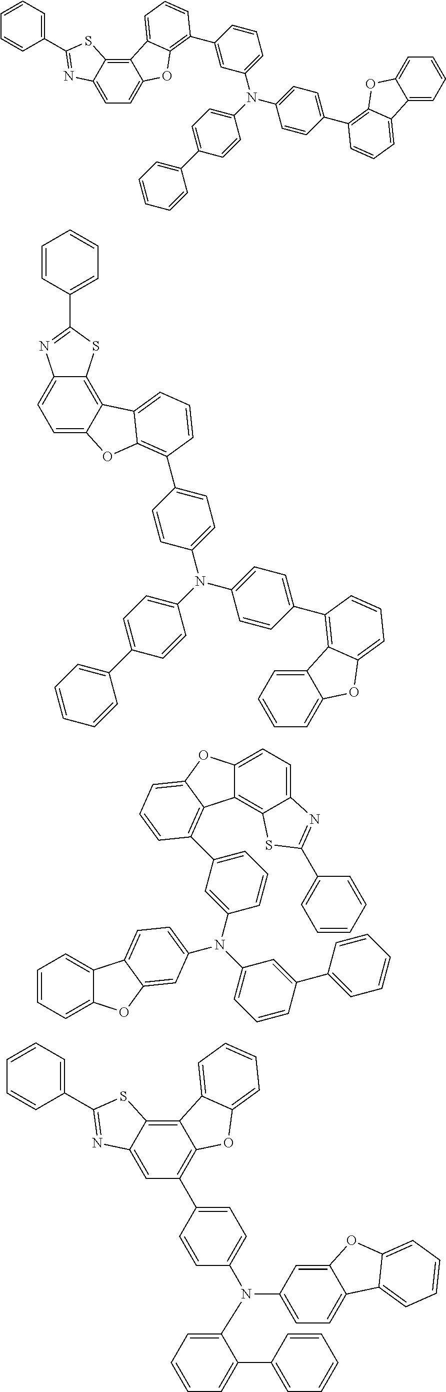

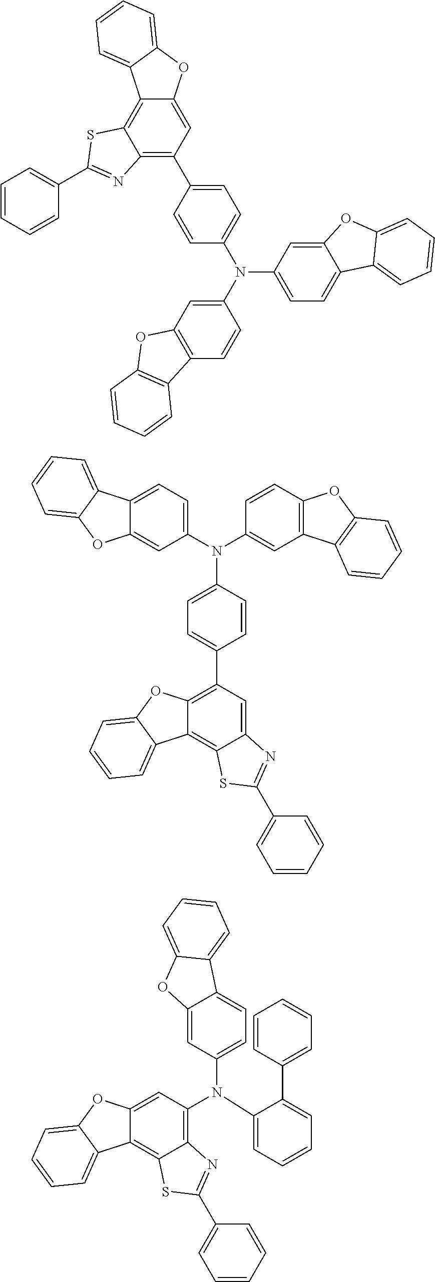 [SMILES: c1ccc(-c2nc3c(-c4ccc(N(c5ccc6c(c5)oc5ccccc56)c5ccc6c(c5)oc5ccccc56)cc4)cc4oc5ccccc5c4c3s2)cc1 ; c1ccc(-c2nc3cc(-c4ccc(N(c5ccc6c(c5)oc5ccccc56)c5ccc6oc7ccccc7c6c5)cc4)c4oc5ccccc5c4c3s2)cc1 ; c1ccc(-c2nc3c(N(c4ccc5c(c4)oc4ccccc45)c4ccccc4-c4ccccc4)cc4oc5ccccc5c4c3s2)cc1]

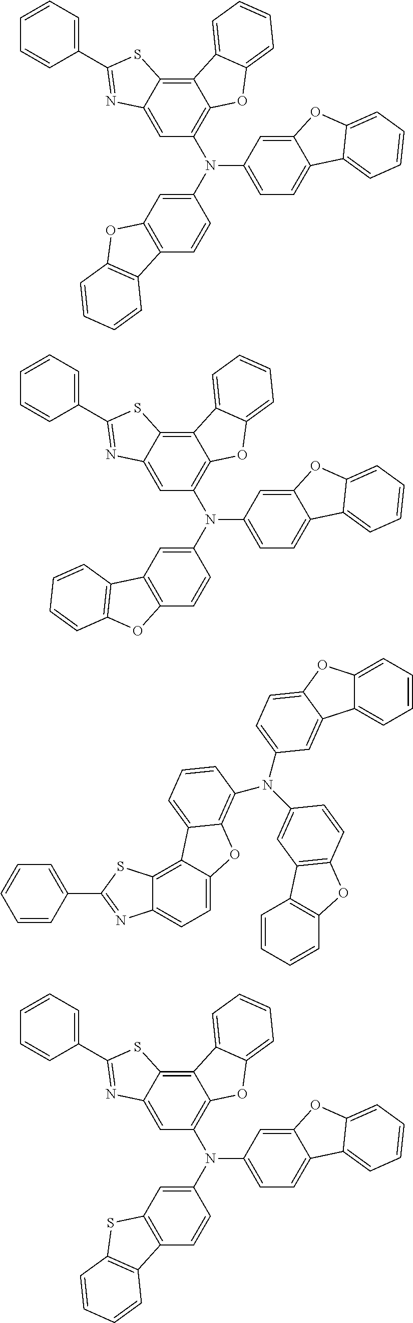 [SMILES: c1ccc(-c2nc3cc(N(c4ccc5c(c4)oc4ccccc45)c4ccc5c(c4)oc4ccccc45)c4oc5ccccc5c4c3s2)cc1 ; c1ccc(-c2nc3cc(N(c4ccc5c(c4)oc4ccccc45)c4ccc5oc6ccccc6c5c4)c4oc5ccccc5c4c3s2)cc1 ; c1ccc(-c2nc3ccc4oc5c(N(c6ccc7oc8ccccc8c7c6)c6ccc7oc8ccccc8c7c6)cccc5c4c3s2)cc1 ; c1ccc(-c2nc3cc(N(c4ccc5c(c4)oc4ccccc45)c4ccc5c(c4)sc4ccccc45)c4oc5ccccc5c4c3s2)cc1]

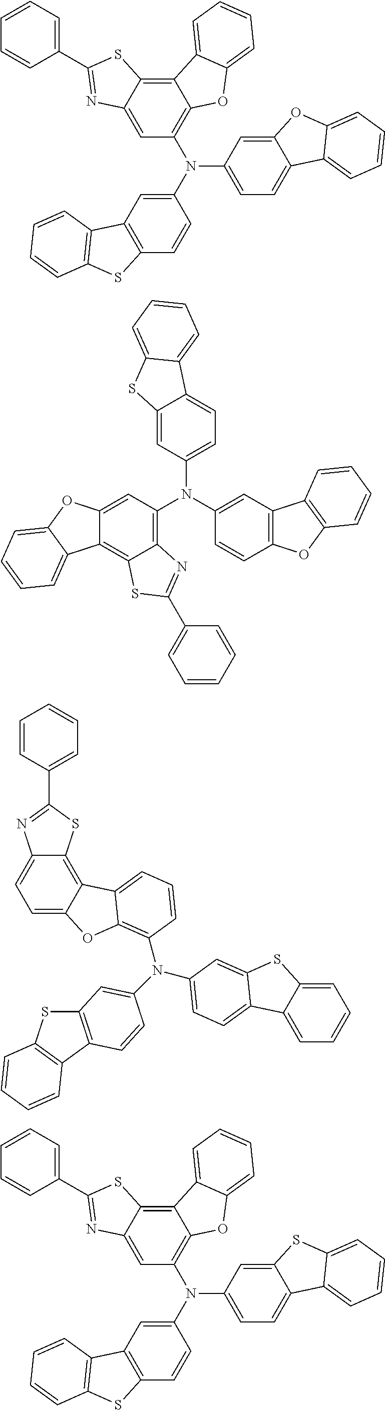

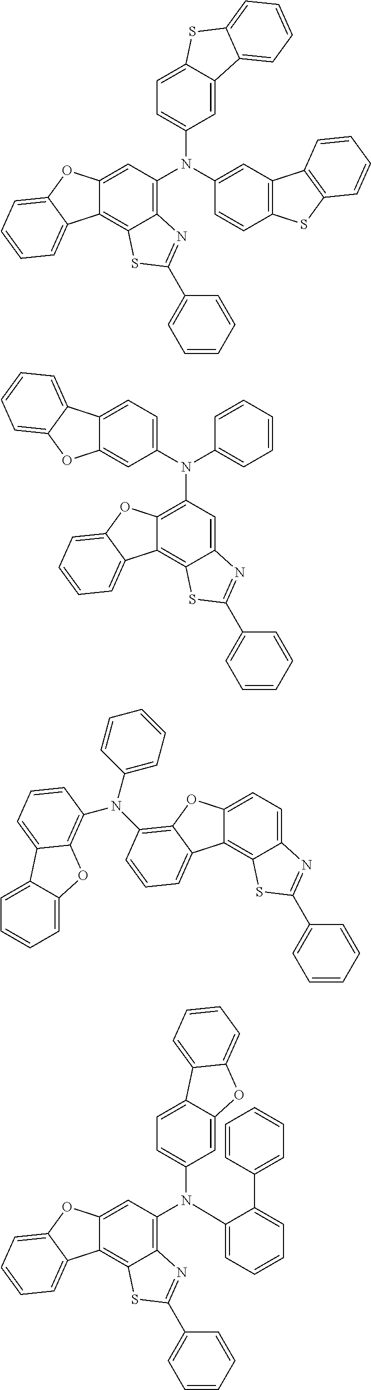 [SMILES: c1ccc(-c2nc3c(N(c4ccc5sc6ccccc6c5c4)c4ccc5sc6ccccc6c5c4)cc4oc5ccccc5c4c3s2)cc1 ; c1ccc(-c2nc3cc(N(c4ccccc4)c4ccc5c(c4)oc4ccccc45)c4oc5ccccc5c4c3s2)cc1 ; c1ccc(-c2nc3ccc4oc5c(N(c6ccccc6)c6cccc7c6oc6ccccc67)cccc5c4c3s2)cc1 ; c1ccc(-c2nc3c(N(c4ccc5c(c4)oc4ccccc45)c4ccccc4-c4ccccc4)cc4oc5ccccc5c4c3s2)cc1]

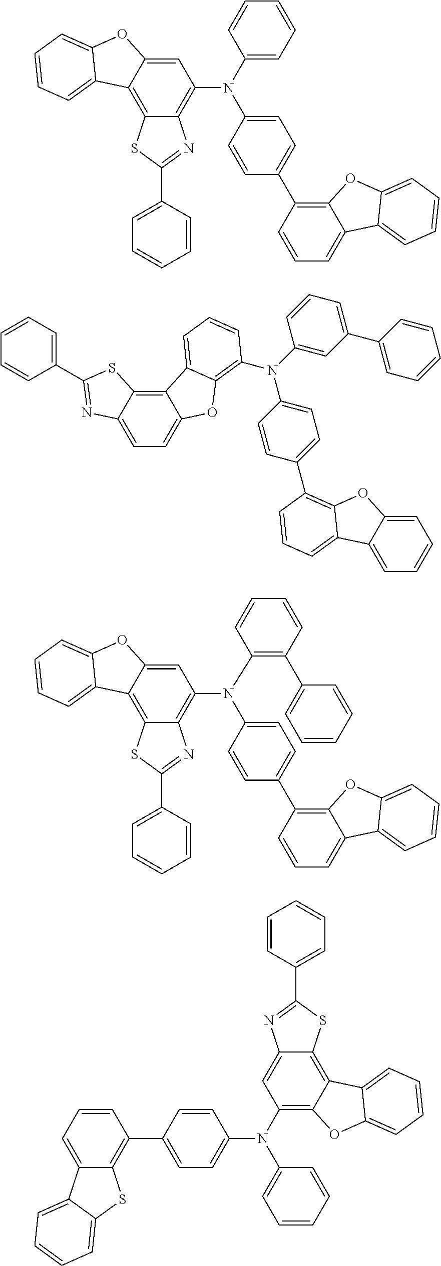

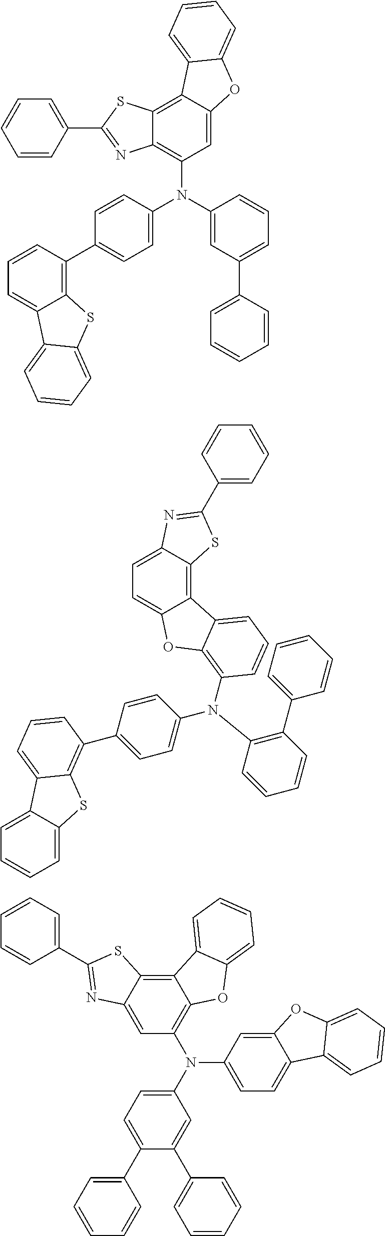

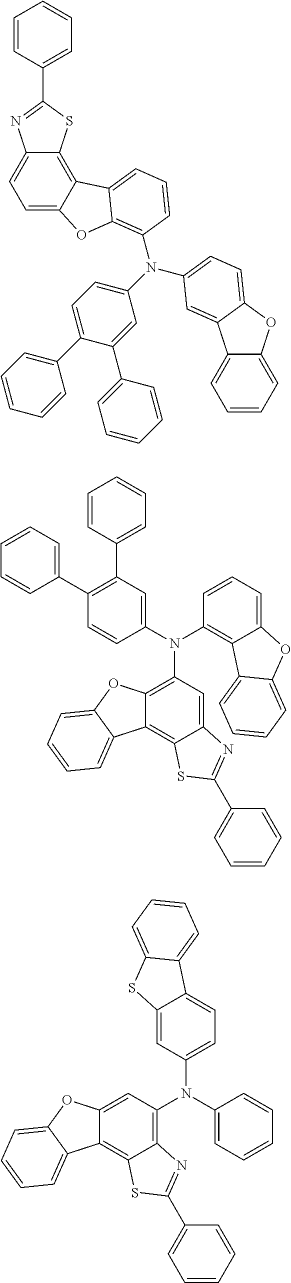

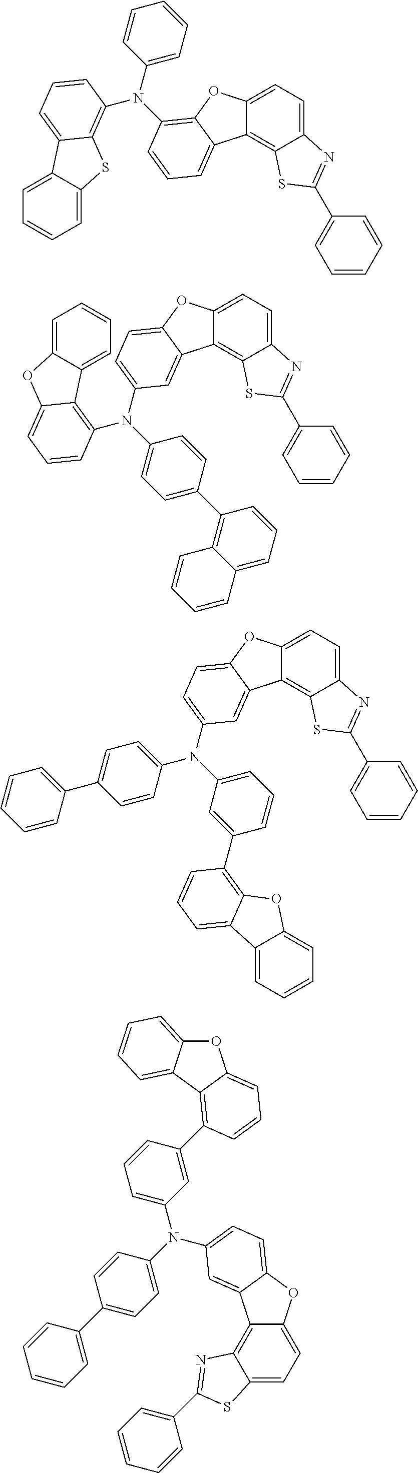

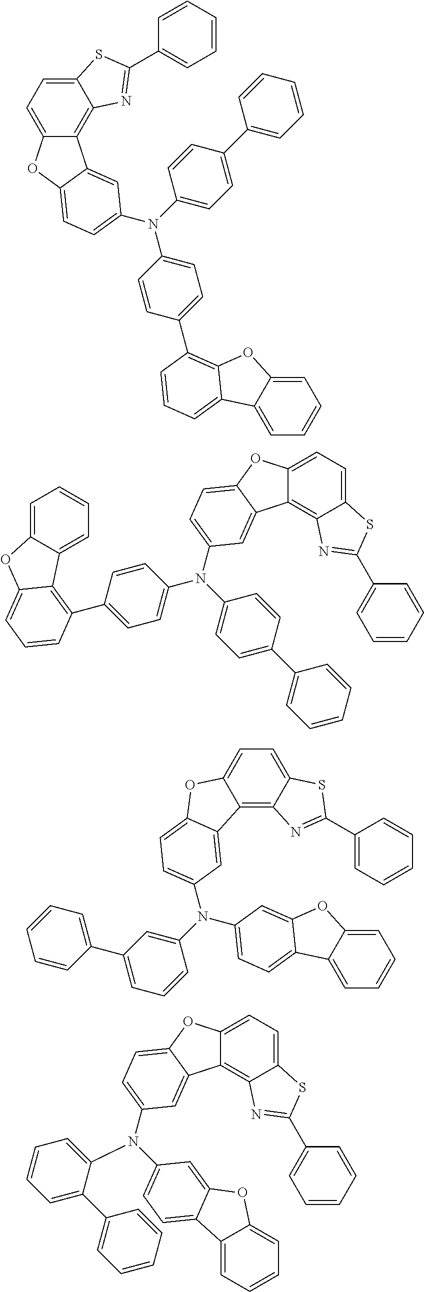

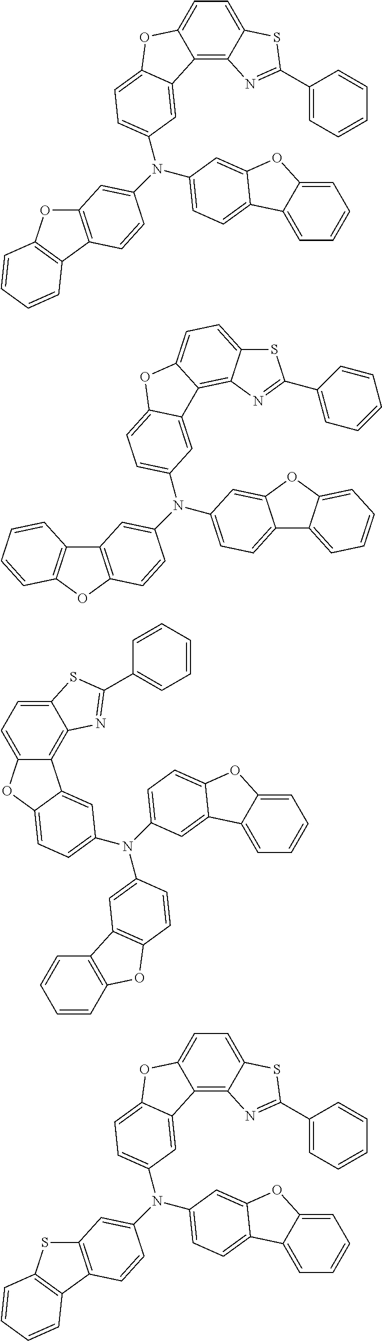

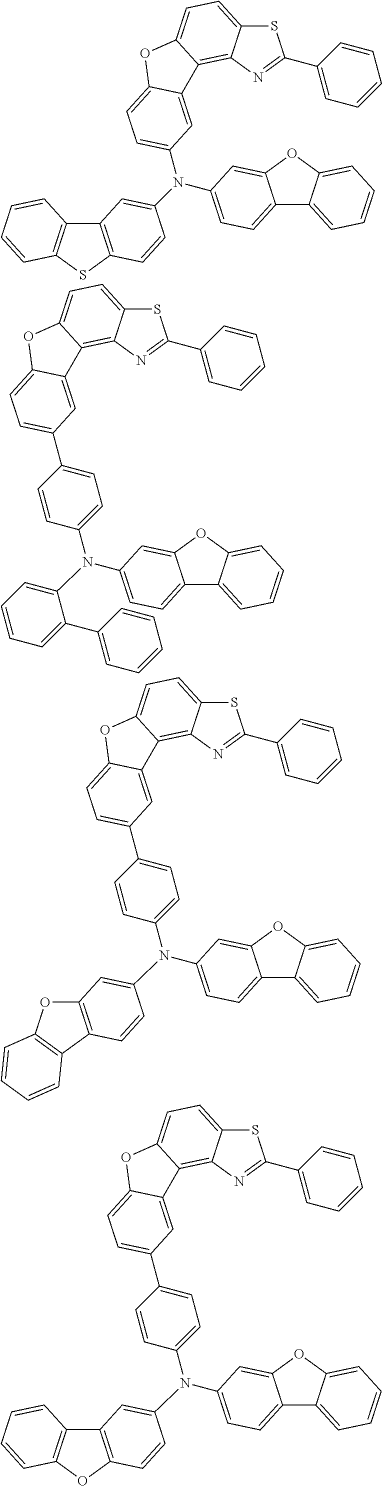

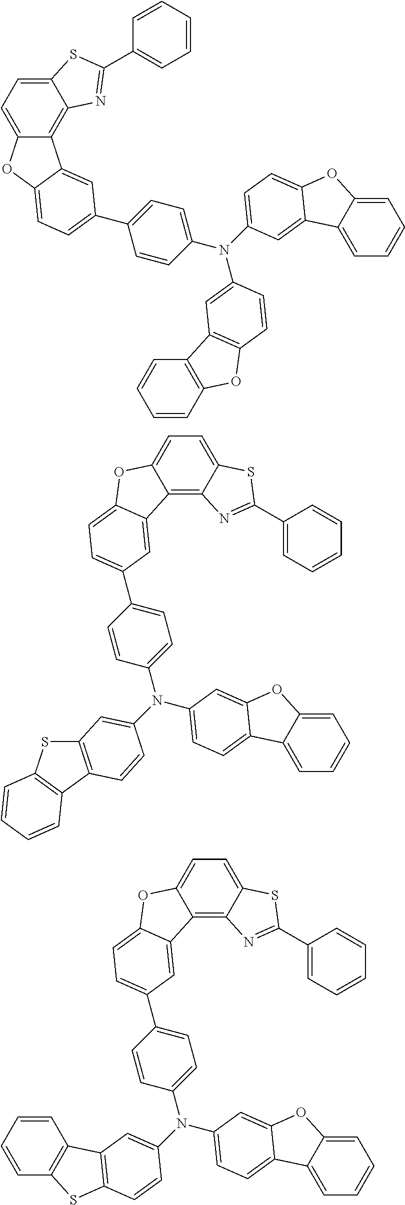

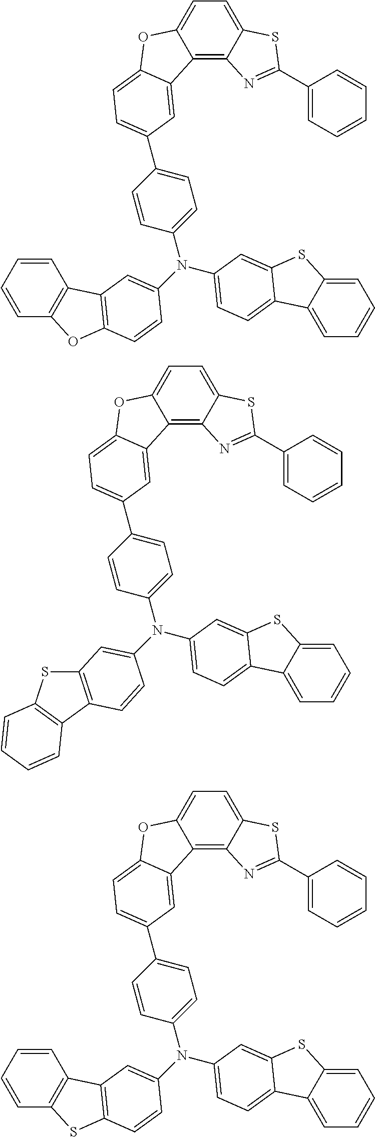

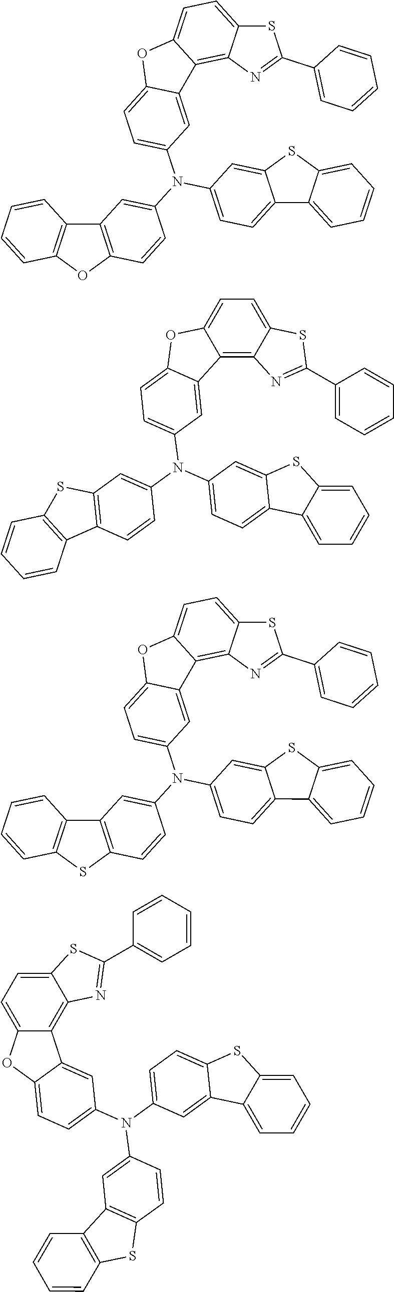

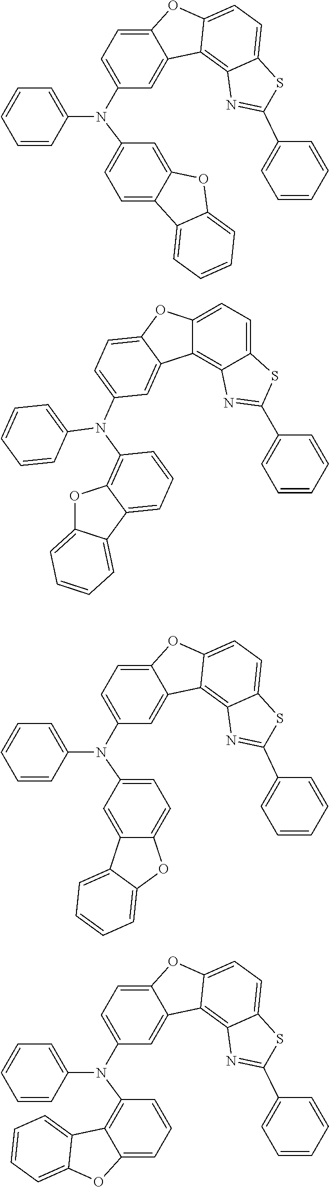

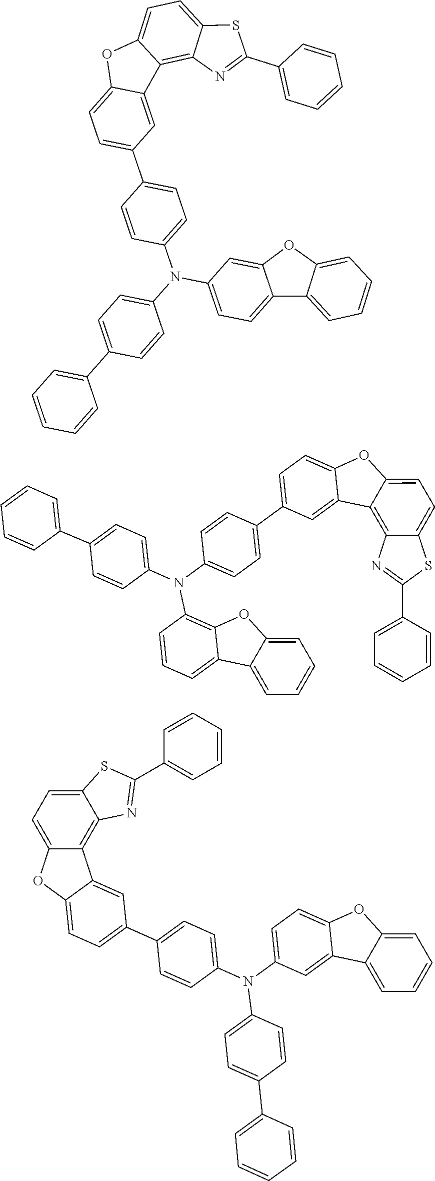

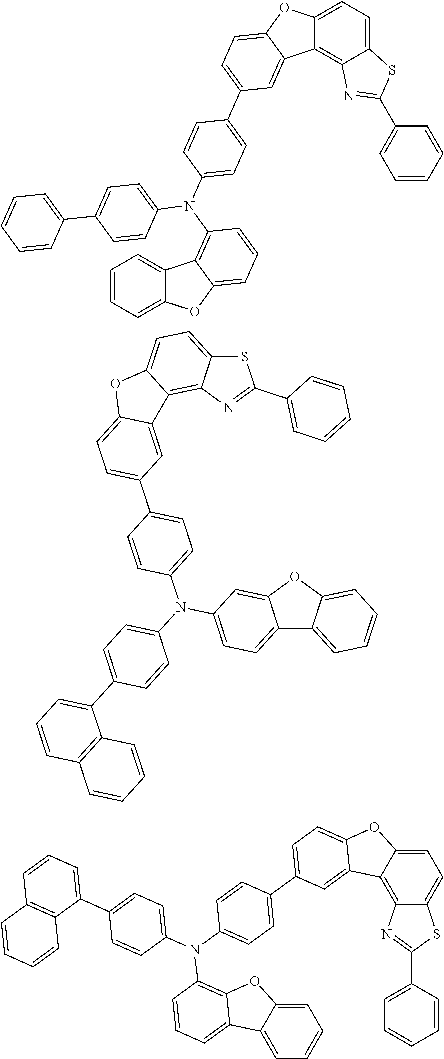

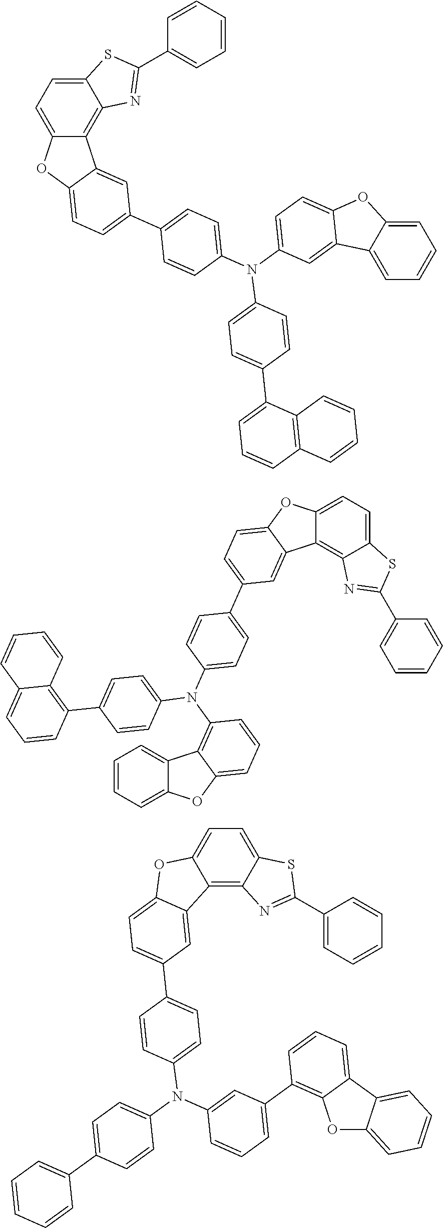

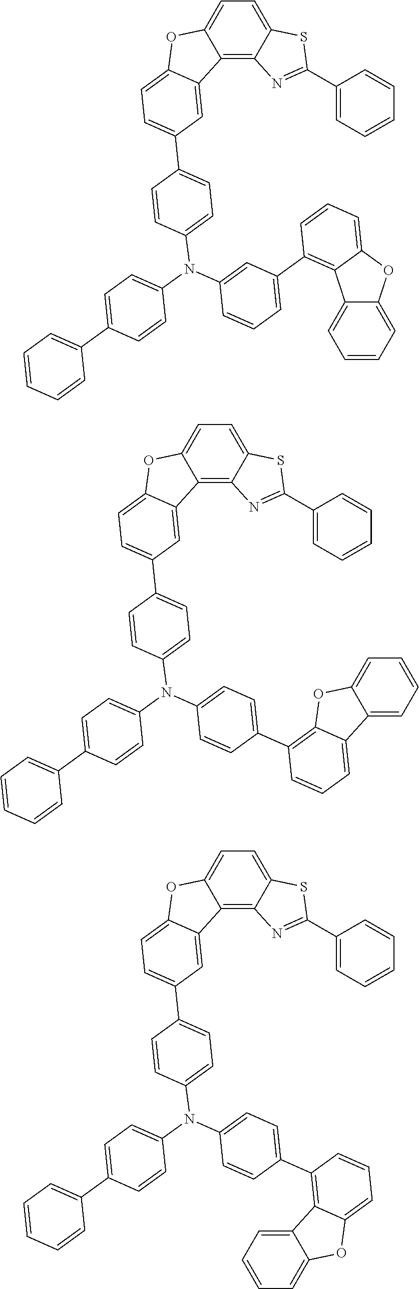 [SMILES: c1ccc(-c2ccc(N(c3ccc(-c4ccc5oc6ccc7sc(-c8ccccc8)nc7c6c5c4)cc3)c3cccc(-c4cccc5oc6ccccc6c45)c3)cc2)cc1 ; c1ccc(-c2ccc(N(c3ccc(-c4ccc5oc6ccc7sc(-c8ccccc8)nc7c6c5c4)cc3)c3ccc(-c4cccc5c4oc4ccccc45)cc3)cc2)cc1 ; c1ccc(-c2ccc(N(c3ccc(-c4ccc5oc6ccc7sc(-c8ccccc8)nc7c6c5c4)cc3)c3ccc(-c4cccc5oc6ccccc6c45)cc3)cc2)cc1]

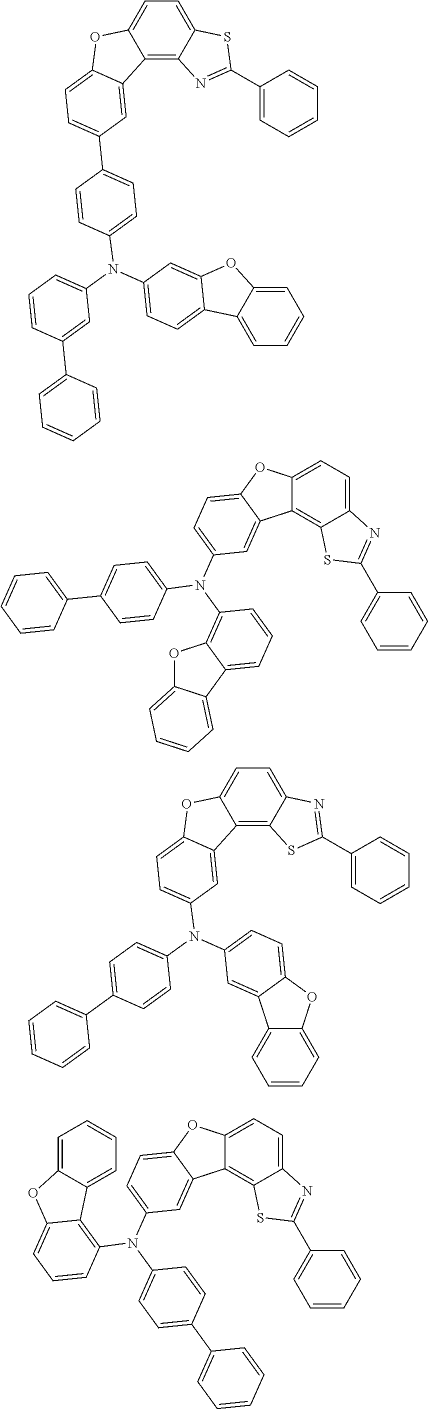

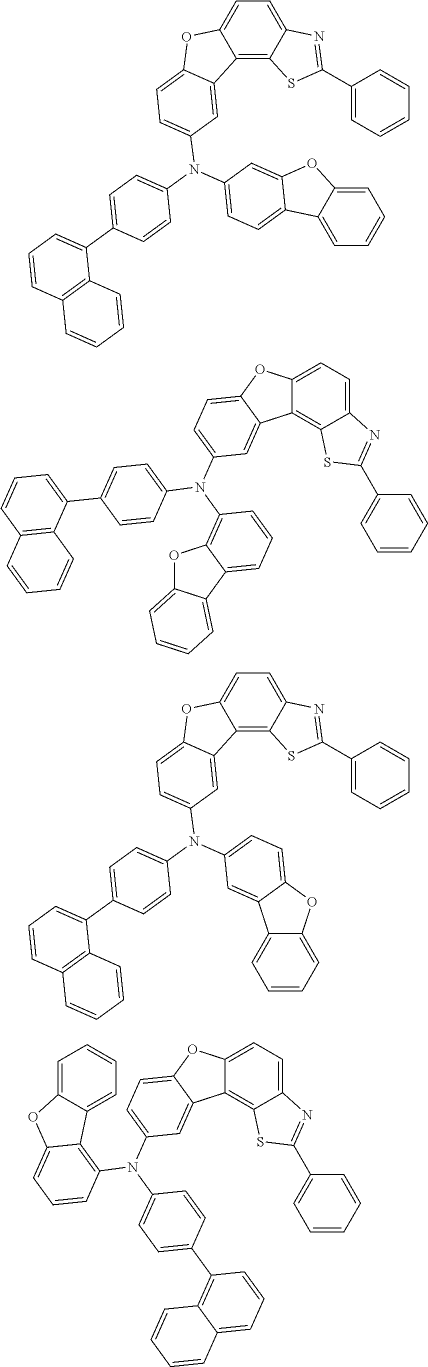

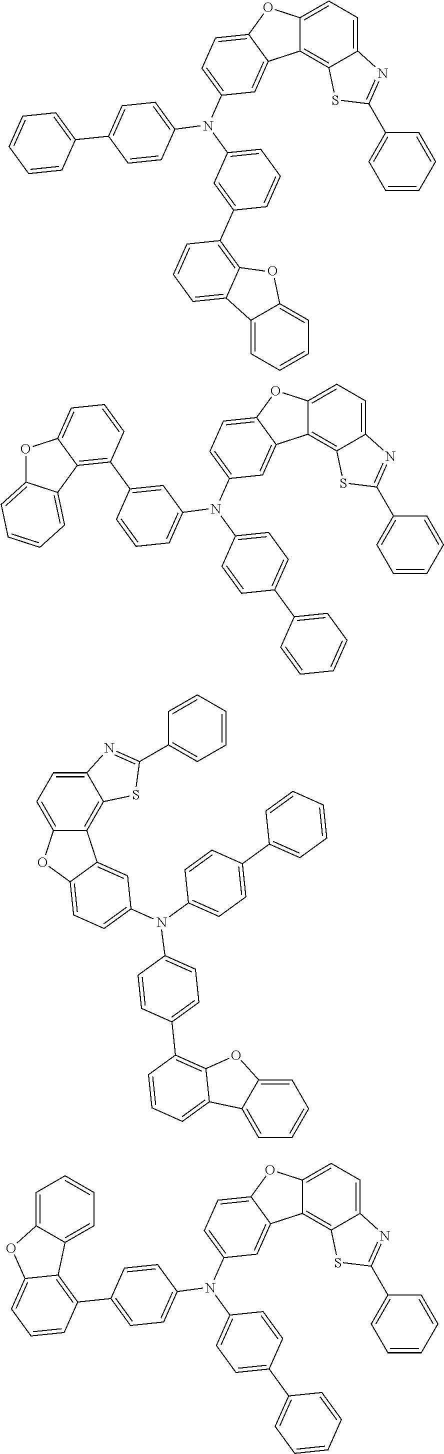

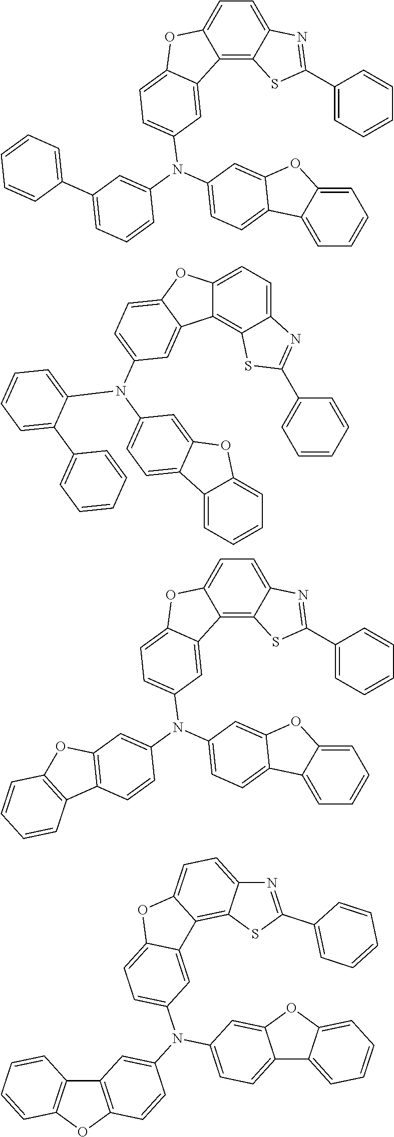

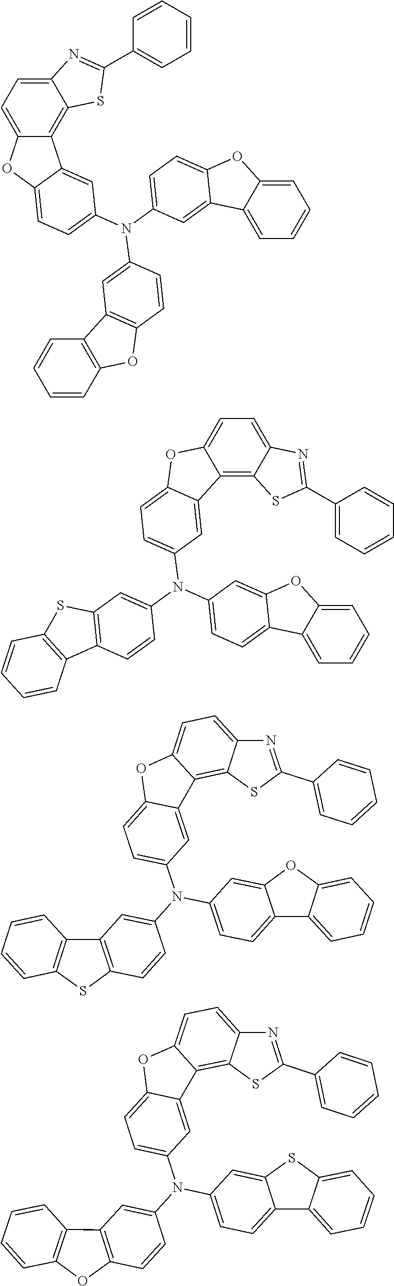 [SMILES: c1ccc(-c2nc3ccc4oc5ccc(N(c6ccc7oc8ccccc8c7c6)c6ccc7oc8ccccc8c7c6)cc5c4c3s2)cc1 ; c1ccc(-c2nc3ccc4oc5ccc(N(c6ccc7c(c6)oc6ccccc67)c6ccc7c(c6)sc6ccccc67)cc5c4c3s2)cc1 ; c1ccc(-c2nc3ccc4oc5ccc(N(c6ccc7c(c6)oc6ccccc67)c6ccc7sc8ccccc8c7c6)cc5c4c3s2)cc1 ; c1ccc(-c2nc3ccc4oc5ccc(N(c6ccc7c(c6)sc6ccccc67)c6ccc7oc8ccccc8c7c6)cc5c4c3s2)cc1]

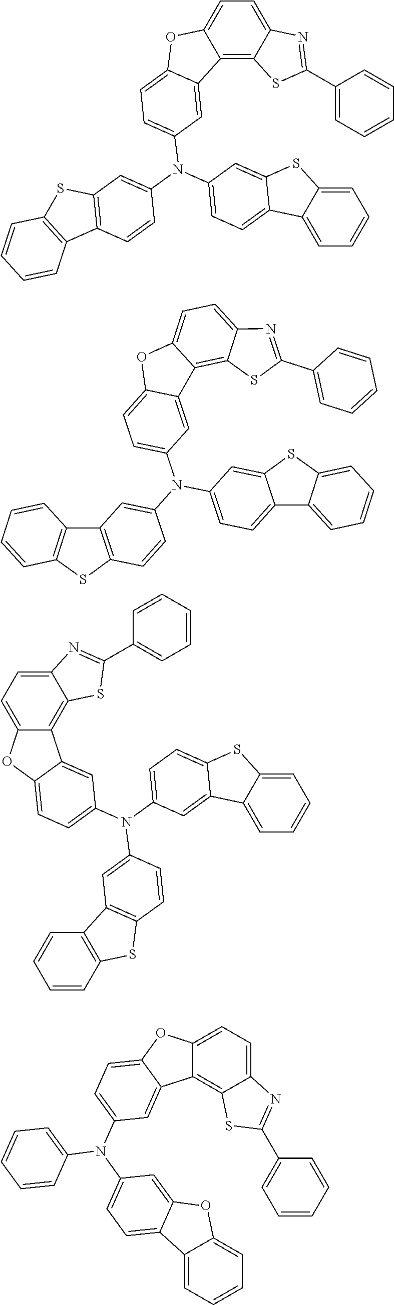

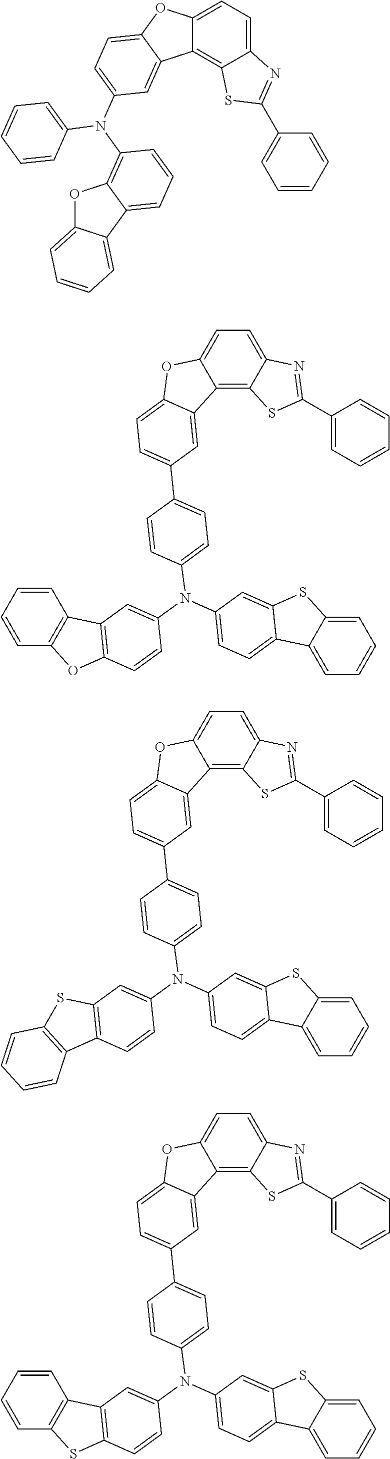 [SMILES: c1ccc(-c2nc3ccc4oc5ccc(N(c6ccccc6)c6cccc7c6oc6ccccc67)cc5c4c3s2)cc1 ; c1ccc(-c2nc3ccc4oc5ccc(-c6ccc(N(c7ccc8c(c7)sc7ccccc78)c7ccc8oc9ccccc9c8c7)cc6)cc5c4c3s2)cc1 ; c1ccc(-c2nc3ccc4oc5ccc(-c6ccc(N(c7ccc8c(c7)sc7ccccc78)c7ccc8c(c7)sc7ccccc78)cc6)cc5c4c3s2)cc1 ; c1ccc(-c2nc3ccc4oc5ccc(-c6ccc(N(c7ccc8c(c7)sc7ccccc78)c7ccc8sc9ccccc9c8c7)cc6)cc5c4c3s2)cc1]

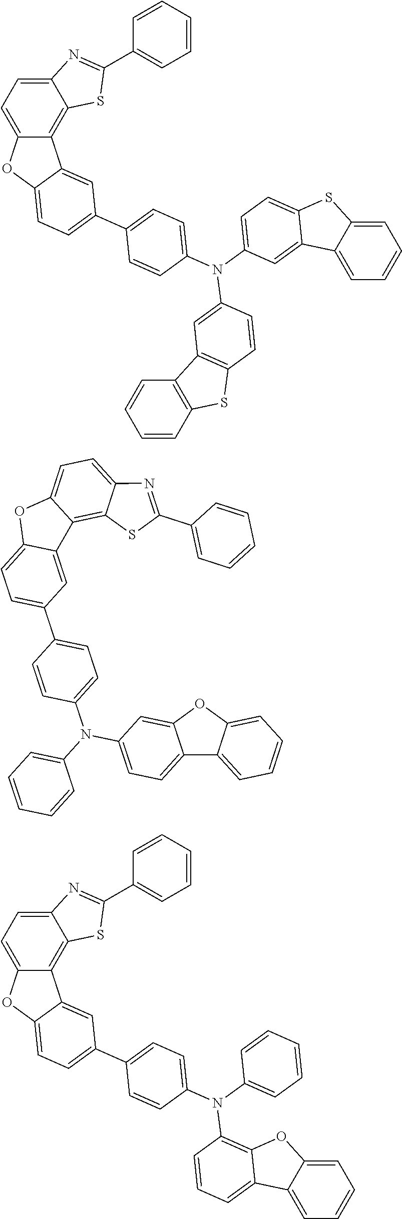

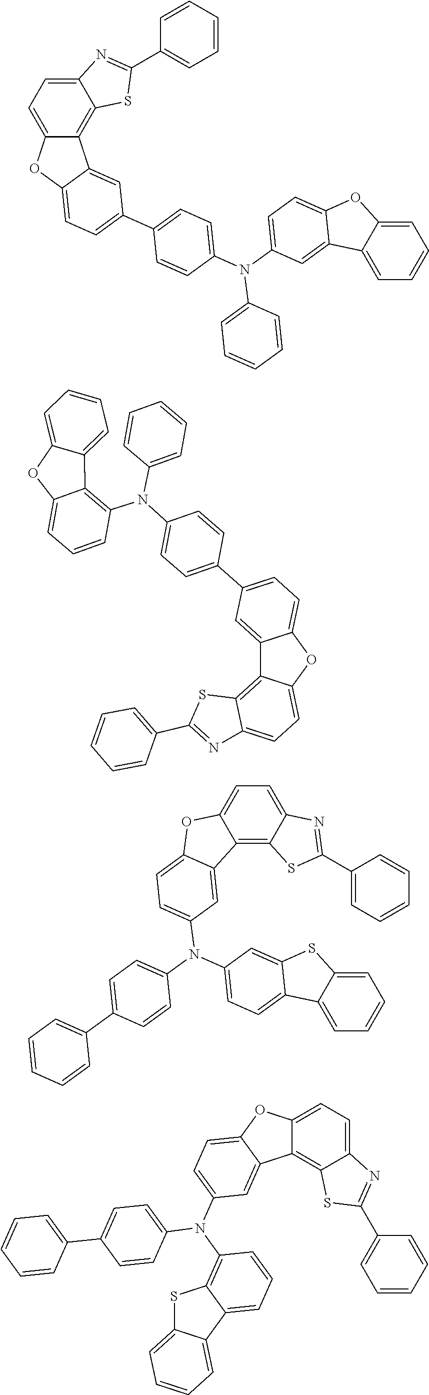

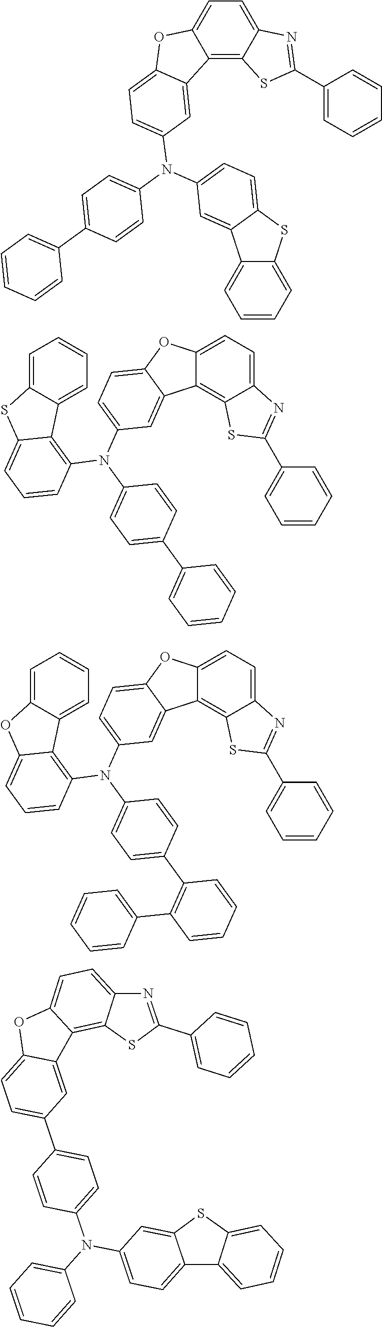

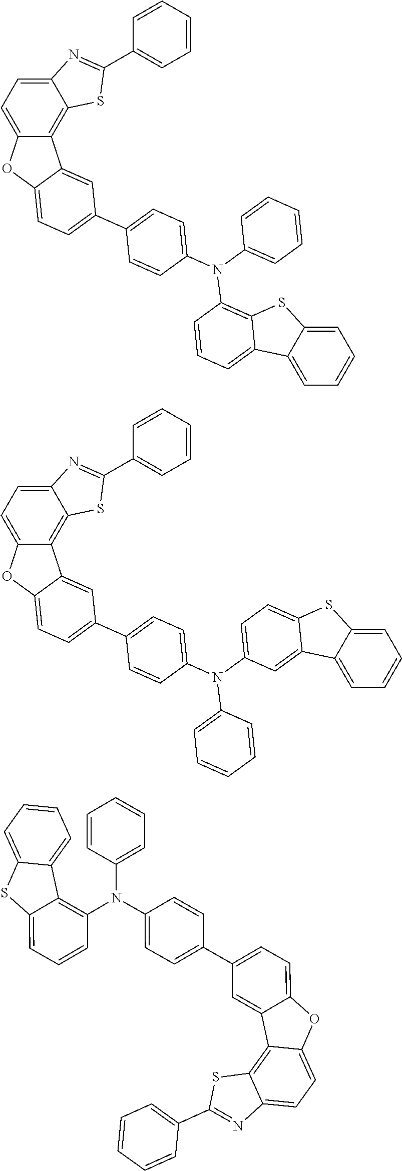 [SMILES: c1ccc(-c2nc3ccc4oc5ccc(-c6ccc(N(c7ccccc7)c7cccc8c7sc7ccccc78)cc6)cc5c4c3s2)cc1 ; c1ccc(-c2nc3ccc4oc5ccc(-c6ccc(N(c7ccccc7)c7ccc8sc9ccccc9c8c7)cc6)cc5c4c3s2)cc1 ; c1ccc(-c2nc3ccc4oc5ccc(-c6ccc(N(c7ccccc7)c7cccc8sc9ccccc9c78)cc6)cc5c4c3s2)cc1]

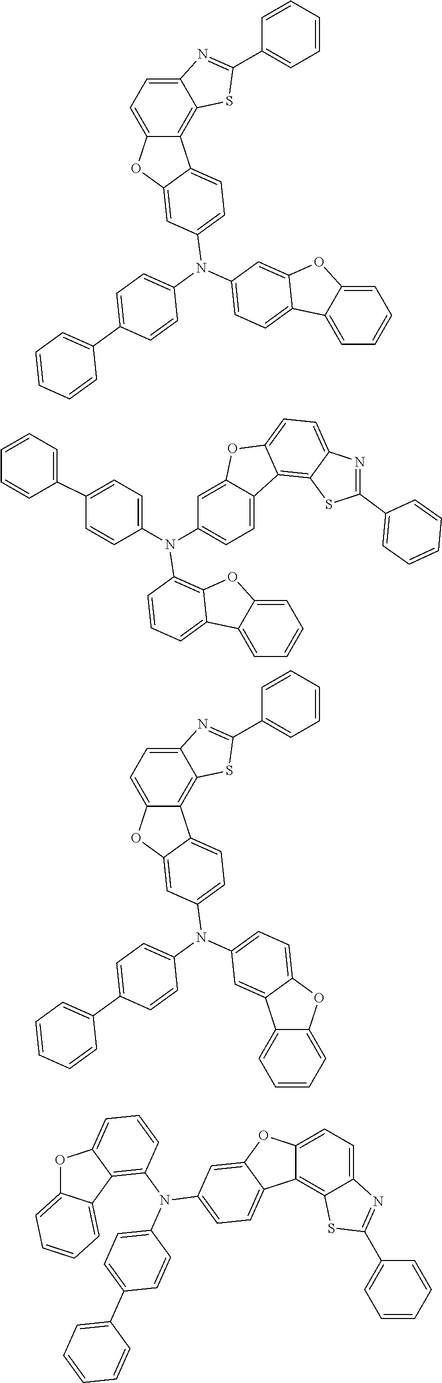

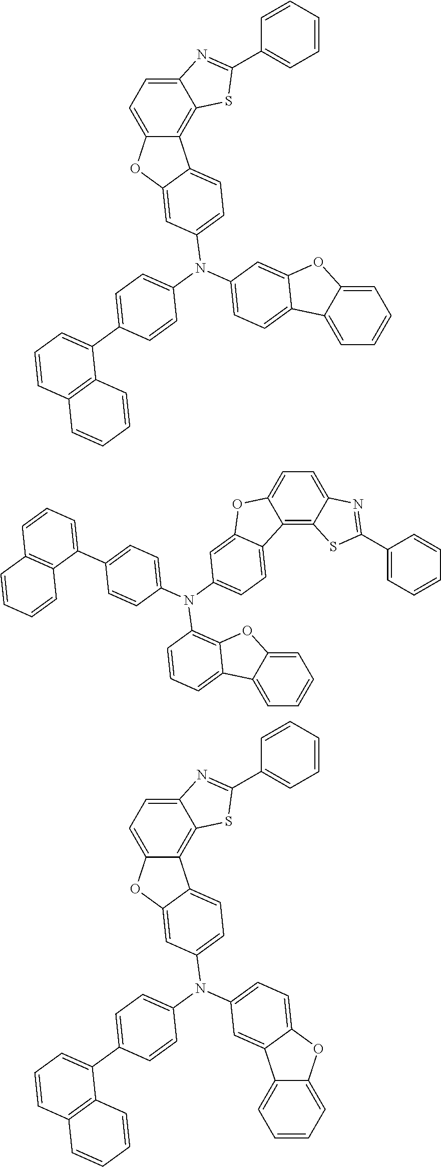

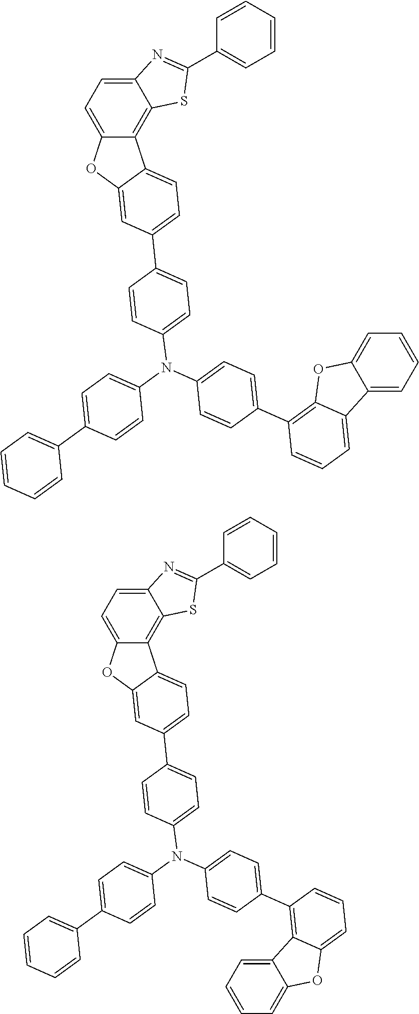 [SMILES: c1ccc(-c2ccc(N(c3ccc(-c4ccc5c(c4)oc4ccc6nc(-c7ccccc7)sc6c45)cc3)c3ccc(-c4cccc5c4oc4ccccc45)cc3)cc2)cc1 ; c1ccc(-c2ccc(N(c3ccc(-c4ccc5c(c4)oc4ccc6nc(-c7ccccc7)sc6c45)cc3)c3ccc(-c4cccc5oc6ccccc6c45)cc3)cc2)cc1]

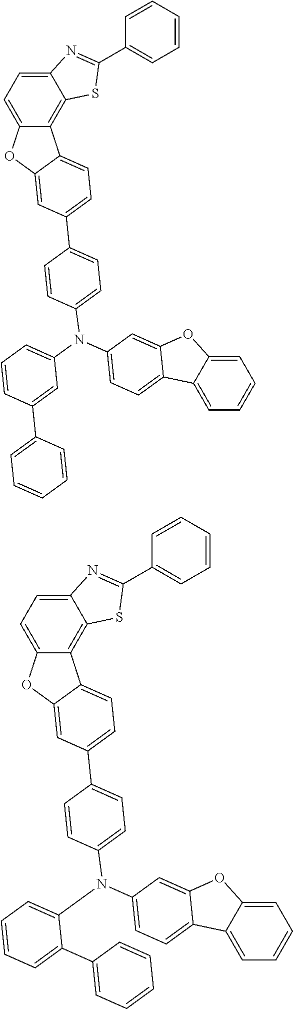 [SMILES: c1ccc(-c2cccc(N(c3ccc(-c4ccc5c(c4)oc4ccc6nc(-c7ccccc7)sc6c45)cc3)c3ccc4c(c3)oc3ccccc34)c2)cc1 ; c1ccc(-c2nc3ccc4oc5cc(-c6ccc(N(c7ccc8c(c7)oc7ccccc78)c7ccccc7-c7ccccc7)cc6)ccc5c4c3s2)cc1]

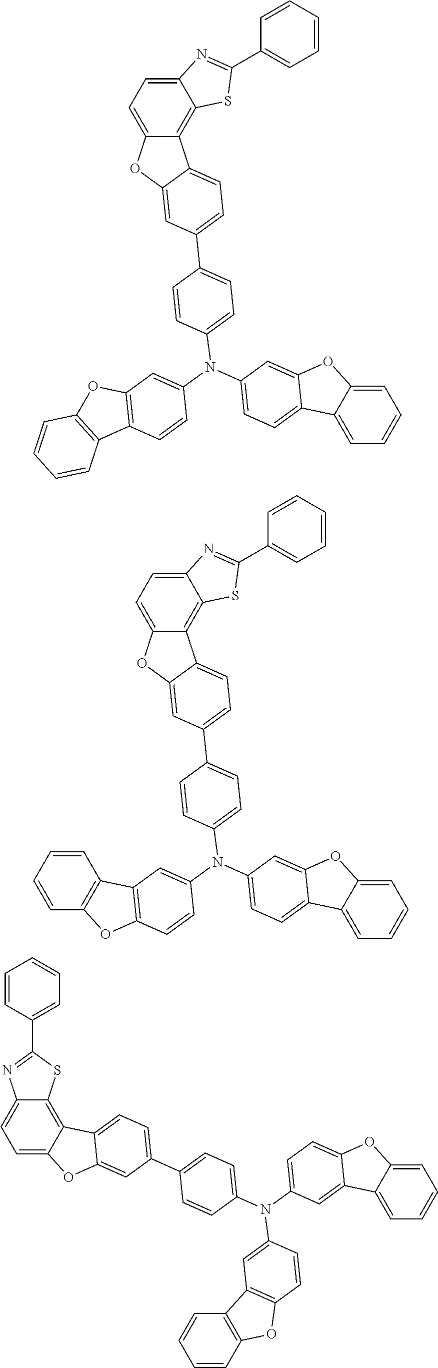 [SMILES: c1ccc(-c2nc3ccc4oc5cc(-c6ccc(N(c7ccc8c(c7)oc7ccccc78)c7ccc8c(c7)oc7ccccc78)cc6)ccc5c4c3s2)cc1 ; c1ccc(-c2nc3ccc4oc5cc(-c6ccc(N(c7ccc8c(c7)oc7ccccc78)c7ccc8oc9ccccc9c8c7)cc6)ccc5c4c3s2)cc1 ; c1ccc(-c2nc3ccc4oc5cc(-c6ccc(N(c7ccc8oc9ccccc9c8c7)c7ccc8oc9ccccc9c8c7)cc6)ccc5c4c3s2)cc1]

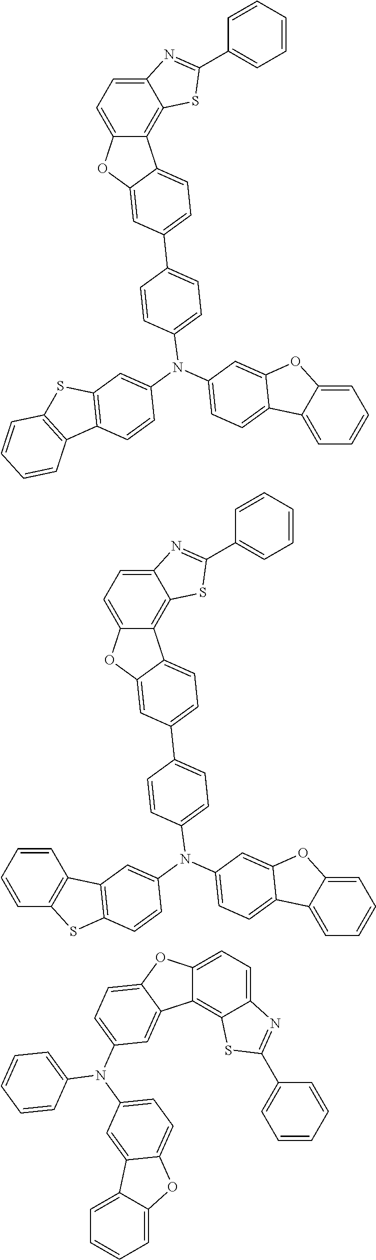

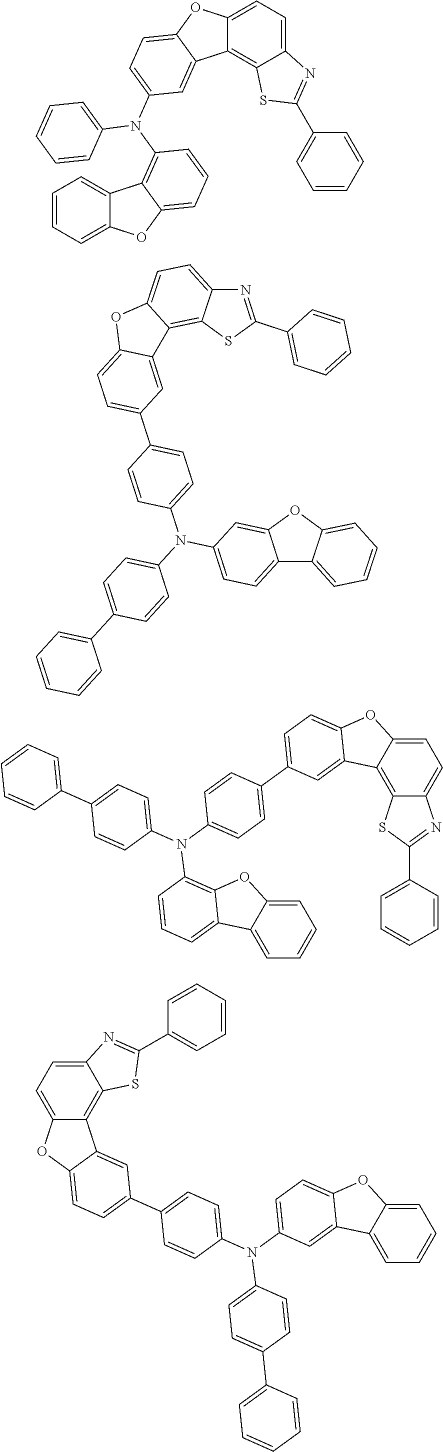

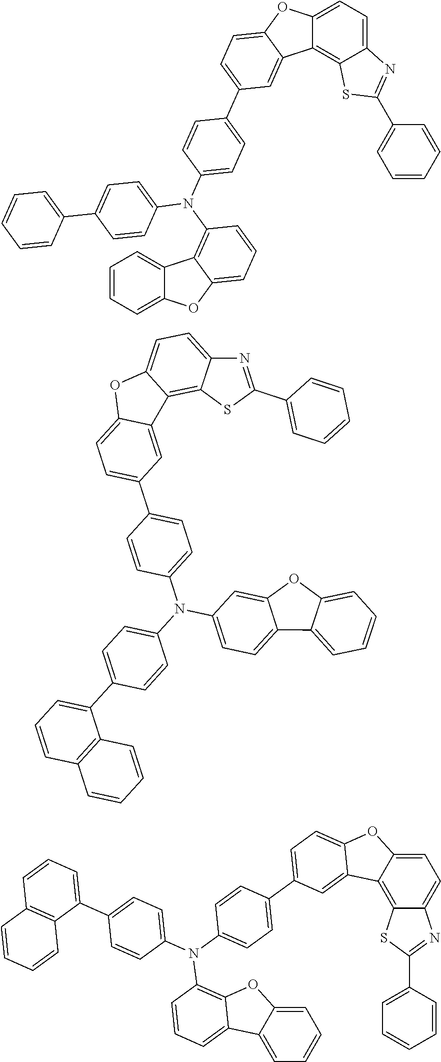

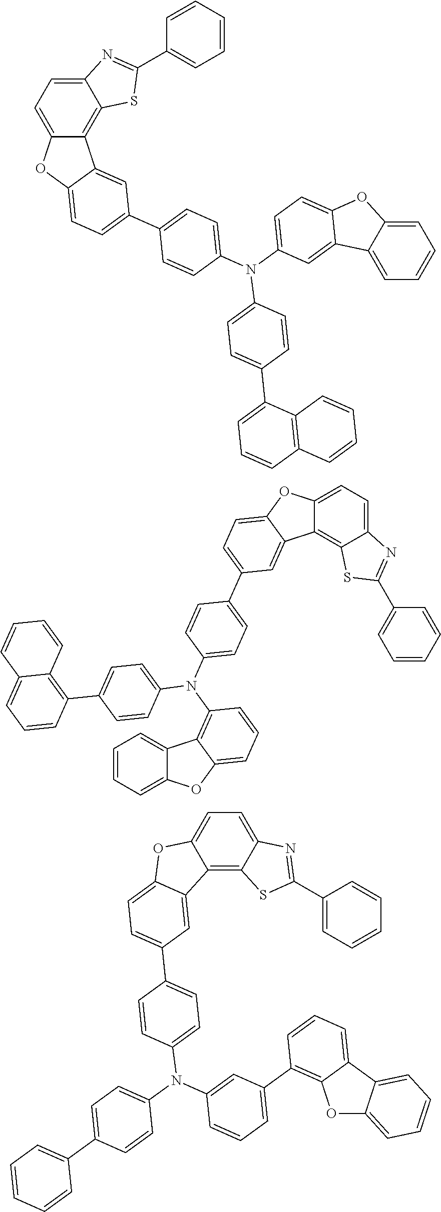

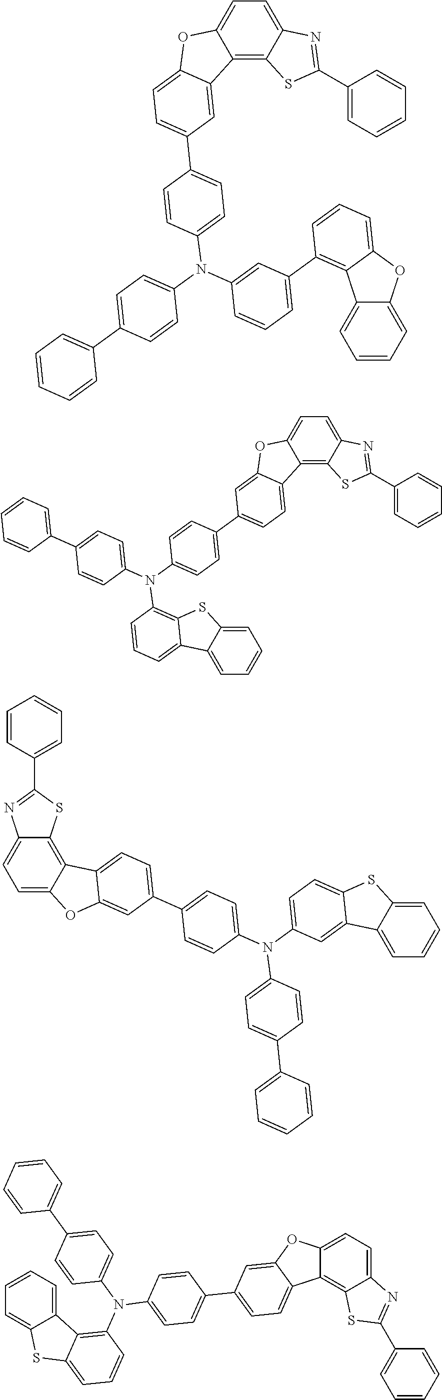 [SMILES: c1ccc(-c2ccc(N(c3ccc(-c4ccc5oc6ccc7nc(-c8ccccc8)sc7c6c5c4)cc3)c3cccc(-c4cccc5oc6ccccc6c45)c3)cc2)cc1 ; c1ccc(-c2ccc(N(c3ccc(-c4ccc5c(c4)oc4ccc6nc(-c7ccccc7)sc6c45)cc3)c3cccc4c3sc3ccccc34)cc2)cc1 ; c1ccc(-c2ccc(N(c3ccc(-c4ccc5c(c4)oc4ccc6nc(-c7ccccc7)sc6c45)cc3)c3ccc4sc5ccccc5c4c3)cc2)cc1 ; c1ccc(-c2ccc(N(c3ccc(-c4ccc5c(c4)oc4ccc6nc(-c7ccccc7)sc6c45)cc3)c3cccc4sc5ccccc5c34)cc2)cc1]

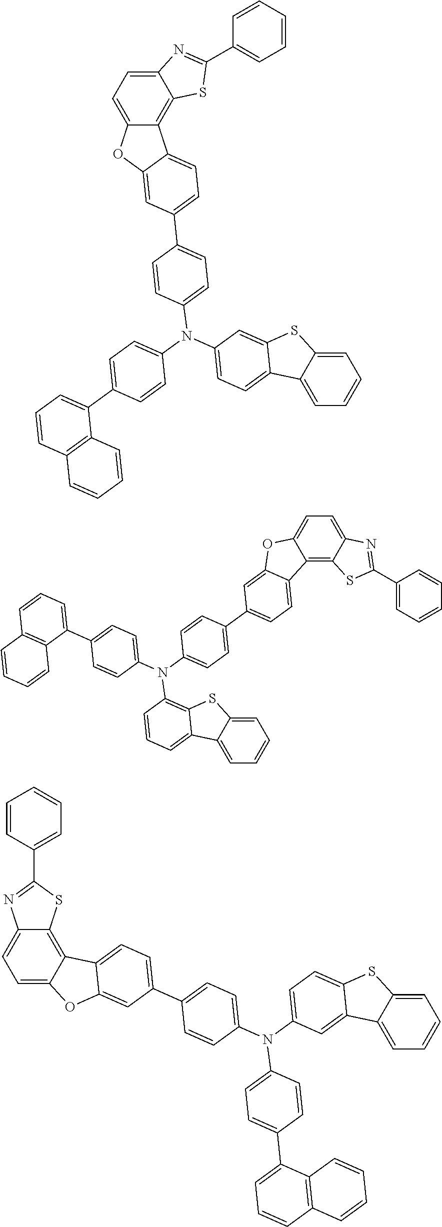 [SMILES: c1ccc(-c2nc3ccc4oc5cc(-c6ccc(N(c7ccc(-c8cccc9ccccc89)cc7)c7ccc8c(c7)sc7ccccc78)cc6)ccc5c4c3s2)cc1 ; c1ccc(-c2nc3ccc4oc5cc(-c6ccc(N(c7ccc(-c8cccc9ccccc89)cc7)c7cccc8c7sc7ccccc78)cc6)ccc5c4c3s2)cc1 ; c1ccc(-c2nc3ccc4oc5cc(-c6ccc(N(c7ccc(-c8cccc9ccccc89)cc7)c7ccc8sc9ccccc9c8c7)cc6)ccc5c4c3s2)cc1]

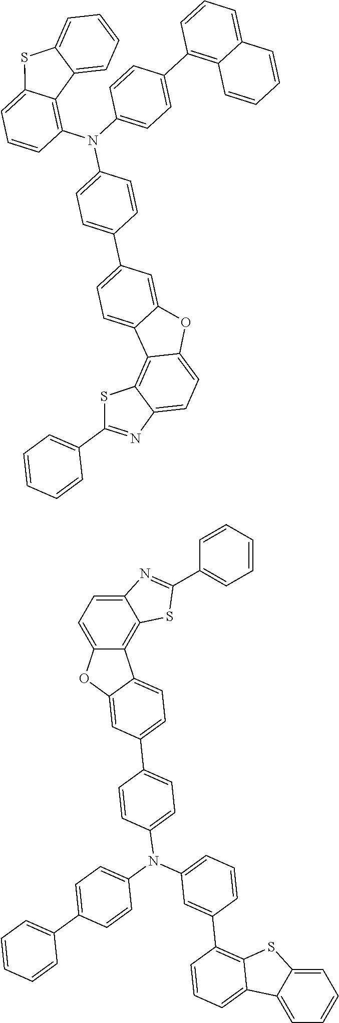 [SMILES: c1ccc(-c2nc3ccc4oc5cc(-c6ccc(N(c7ccc(-c8cccc9ccccc89)cc7)c7cccc8sc9ccccc9c78)cc6)ccc5c4c3s2)cc1 ; c1ccc(-c2ccc(N(c3ccc(-c4ccc5c(c4)oc4ccc6nc(-c7ccccc7)sc6c45)cc3)c3cccc(-c4cccc5c4sc4ccccc45)c3)cc2)cc1]

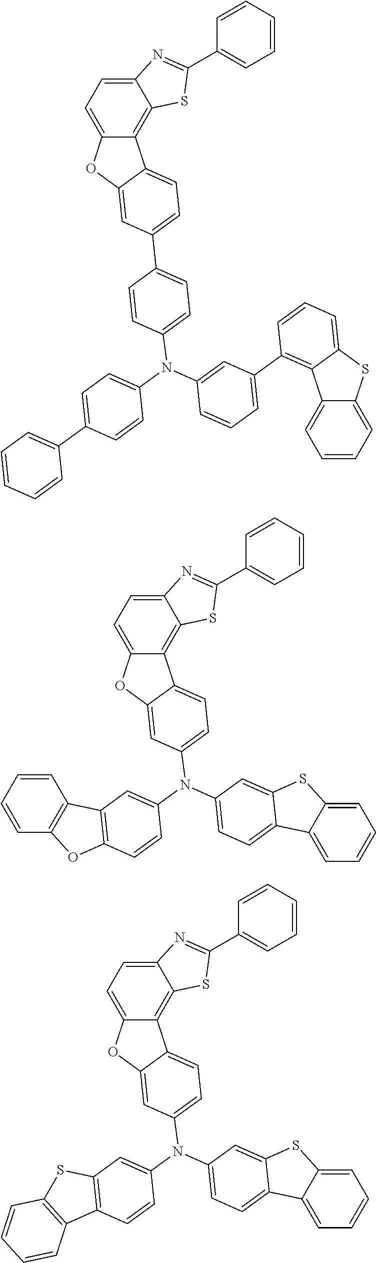

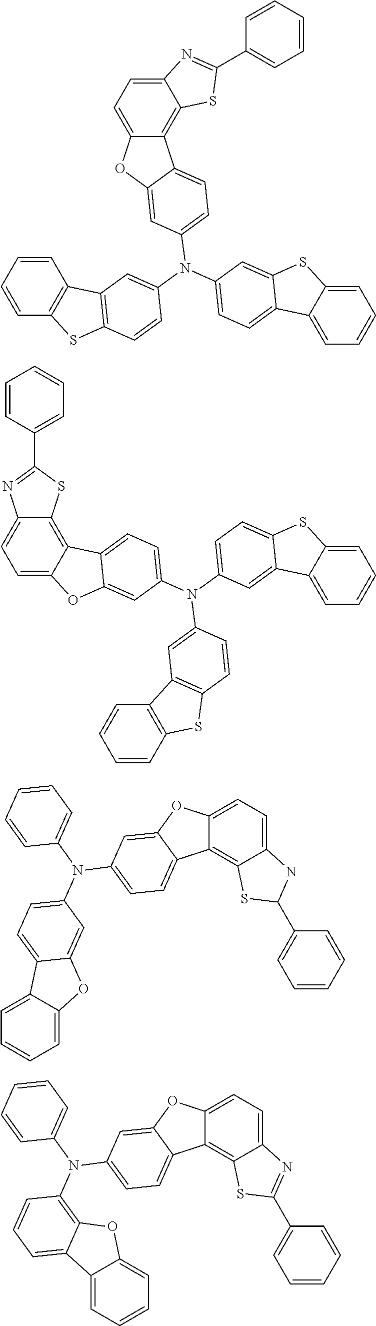

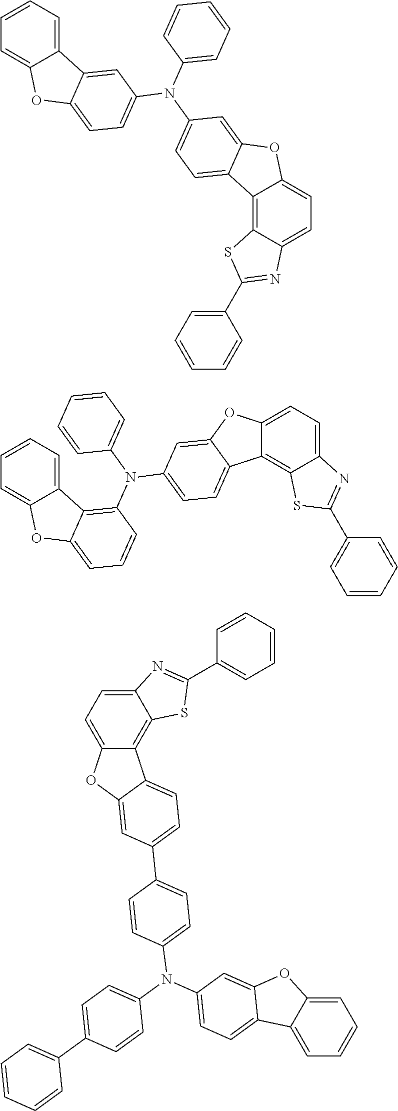

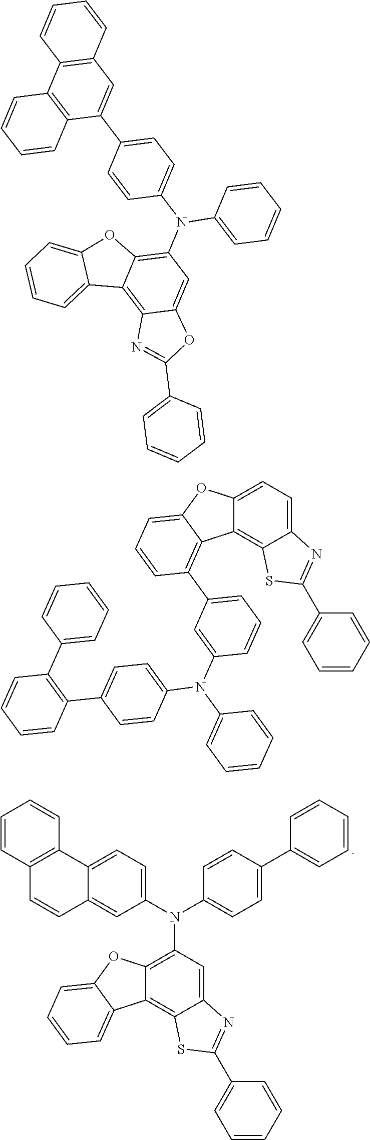

12. An organic light emitting device comprising:
a first electrode;
a second electrode that is provided opposite to the first electrode; and
one or more organic material layers that are provided between the first electrode and the second electrode, wherein at least one layer of the organic material layers comprises at least one compound according to claim 1.

13. The organic light emitting device of claim 12, wherein the organic material layer is a light emitting layer or an electron blocking layer.
